(19) United States
(12) Patent Application Publication
(10) Pub. No.: US 2026/0216245 A1
(43) Pub. Date: Jul. 30, 2026

(54) THERAPEUTIC BASE EDITING

(71) Applicants: The J. David Gladstone Institute, A Testamentary Trust Established Under the Will of J. David Gladst, San Francisco, CA (US); The Regents of The University of California, Oakland, CA (US)

(72) Inventors: Alexander Marson, San Francisco, CA (US); Carl Ward, San Francisco, CA (US); Ralf Schmidt, Bernsdorf Saxony (DE)

(21) Appl. No.: 19/151,579

(22) PCT Filed: Feb. 7, 2024
(86) PCT No.: PCT/US2024/014756

§ 371 Date: Jul. 28, 2025

Related U.S. Application Data
(60) Provisional application No. 63/443,822, filed on Feb. 7, 2023.
(60) Provisional application No. 63/491,664, filed on Mar. 22, 2023.

(51) Int. Cl. A61K 35/17; A61K 40/11; A61K 40/31; A61K 40/42; A61P 35/00; C07K 14/525; C07K 14/55; C07K 14/57; C12N 5/0783; C12N 9/78; C12N 15/11; C12N 15/86

(52) U.S. Cl. CPC A61K 35/17; A61K 40/11; A61K 40/31; A61K 40/4232; A61K 40/4234; A61K 40/4235; A61P 35/00; C07K 14/525; C07K 14/55; C07K 14/57; C12N 5/0636; C12N 9/78; C12N 15/111; C12N 15/86; C12N 2310/20; C12N 2510/00; C12N 2710/10343; C12N 2710/16043; C12N 2740/10043; C12N 2740/15043; C12N 2750/14143; C12Y 305/04002; C12Y 305/04005

(57) ABSTRACT

A method to screen for base edited genes with modifications in therapeutic targets, cells produced by the method and methods of using the modified cells are provided.

### PRIORITY

This application claims priority to and the benefit of U.S. Provisional Patent Application No. 63/443,822, filed Feb. 7, 2023, and to U.S. Provisional Patent Application No. 63/491,664, filed Mar. 22, 2023, the entire disclosures of which are incorporated herein by this reference.

### GOVERNMENT FUNDING

This invention was made with government support under CA245718 awarded by the National Institutes of Health. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Feb. 7, 2024, is named “3730222WO1.xml” and is 518,254 bytes in size.

### BACKGROUND

DNA base-editors encompass two key components: a Cas enzyme for programmable DNA binding and a single-stranded DNA modifying enzyme for targeted nucleotide alteration. Two classes of DNA base-editors have been described: cytosine base-editors and adenine base-editors. Collectively, all four transition mutations (C→T, T→C, A→G, and G→A) can be installed with the available CRISPR/Cas BEs. Two base-editor architectures were described that can efficiently induce targeted C-to-G base transversions. In addition, studies report dual base-editor systems for combinatorial editing in human cells.

### SUMMARY

A highly efficient method was employed to perform pooled Base Editing screens in primary human T cells. This includes but is not limited to adenine base editors (ABE), cytosine base editors (CBE) and with different types of Cas9 (e.g., Cas9 with NGG PAM, SpG Cas9 with NG PAM, or NG Cas9 with NG PAM or SpRY (NRN>NYN PAMs) disclosed in Walton et al., Science, 368:290 (2020), the disclosure of which is incorporated by reference herein). This approach was used to identify genomic regions (at the amino acid level) in coding regions which can be targeted to alter T cell functionality in multiple contexts. Base editing employs a catalytically impaired Cas protein such as one fused to, for example, a deaminase converting A/T to G/C (adenine base editors although editors for other bases are envisioned, see, e.g., Table 1 in Rees and Liu, Nat.Rev.Genet., 19.770 (2019), the disclosure of which is incorporated by reference herein). The enzyme and sgRNAs may be delivered to cells as isolated protein or isolated nucleic acid, or via one or more vectors such as plasmids or viral or virus like vectors or nanoparticles encoding the enzyme or sgRNA, or other delivery vehicles.

Importantly, methods and compositions were used to generate base edited T cells with improved functionality that could enhance immunotherapies. The functionality includes direct responses to T cell activation as well as the integration of environmental factors. A set of screens identified causal relationships between about 117,000 mutation sites in the entire coding region of selected genes and their effect on T cell activation and function.

Therapeutics modulating the function of the immune system can be applied but are not limited to cancer, autoimmunity, infections, rheumatic diseases. Here, a screening approach was used to identify targets that could be used to fine-tune the response of the immune system in all these contexts. For example, the present results can be used to enhance T cell activity against cancer, by precisely activating or inhibiting all or a particular function of specific proteins (e.g., turning off only DNA binding capability of a transcription factor while keeping its ability to form complexes with other TFs or the other way around; alternatively deleting specific binding capabilities of one domain while keeping other domains intact and rewiring the signaling process. This is an advantage over KO or overexpression, due to its precision but also because redundancy loops can't be activated (e.g., there are two kinases doing the same job, one of which is the primary kinase and the other one becomes active when primary kinase is completely knocked out. With base editing, only kinase function would be impaired (or enhanced) while the protein itself would still be expressed and mechanisms activating the alternative kinase would not become active)). Due to the highly multiplexable nature of Base Editing, the identified hits/targets can be readily combined with each other or other genetic perturbations to further increase their potency or integrated into existing therapies.

The disclosure thus provides for a method for high throughput base editing screens in primary human T cells using a virus such as a lentivirus including an optimized lentiviral transfer plasmid architecture that allows for high titer virus production and consequently delivery to primary human T cells. Also provided is an approach for high throughput screening of protein/gene domains at amino acid level, in primary human T cells. A large set of previously uncharacterized targets that alter T cell function were identified.

Methods to make base edited T cells with enhanced functionality in the resulting T cells are provided. Those cells allow for the ability to dissect different (including opposing) functional regions within a protein and exploitation for therapeutic or diagnostic purposes.

As described herein, lentiviral enabled pooled base editor screens can be used in many contexts to understand the function of the 100k+ variants in various model systems. The size of the screening library can be expanded or the targets the screening library can be altered to target and investigate different genes or non-coding genomic regions (e.g., those known to be relevant to the disease/phenotype of interest), thereby facilitating discovery of therapeutically relevant functional sites for T cells. The hits can be used for detailed investigation and tested for biologic activity in specific disease models. Successful candidates are further tested for multiplexed therapeutic strategies. For example, an identified mutation can be introduced into anti-cancer T cells to enhance their potency or to anchor sites that can lead to enhanced potency.

In one embodiment, the genes in FIGS. 1-4 are modified in cells, e.g., immune cells such as T cells. In one embodiment, sgRNAs having at least 80%, 82%, 85%, 87%, 88%, 89%, 90%, 92%, 94%, 95%, 96%, 97%, 98% or 99% nucleic acid sequence identity to any one of sgRNAs provided herein are used to modify cells, e.g., immune cells. In one embodiment, sites having at least 80%, 82%, 85%, 87%, 88%, 89%, 90%, 92%, 94%, 95%, 96%, 97%, 98% or 99% nucleic acid sequence identity to any one of those disclosed in FIG. 1, 2, 3 or 4 in cells are modified.

In one embodiment, the genes in FIG. 11 are modified in cells, e.g., immune cells such as T cells. In one embodiment, sgRNAs having at least 80%, 82%, 85%, 87%, 88%, 89%, 90%, 92%, 94%, 95%, 96%, 97%, 98% or 99% nucleic acid sequence identity to any one of sgRNAs provided herein are used to modify cells, e.g., immune cells. In one embodiment, sites having at least 80%, 82%, 85%, 87%, 88%, 89%, 90%, 92%, 94%, 95%, 96%, 97%, 98% or 99% nucleic acid sequence identity to any one of those disclosed in FIG. 11 in cells are modified.

In some embodiments, the gene is 1. PIK3CD, VAV1, LCP2, PLCG1, DGKZ or having at least 80%, 82%, 85%, 87%, 88%, 89%, 90%, 92%, 94%, 95%, 96%, 97%, 98% or 99% nucleic acid sequence identity thereto. In some embodiments, guides are selected from one or more of PLCG1_pos: TGACCAGAATTTCCTGGCTC; DGKZ_pos: AGGGGCAGGATGGCAACAGG; pos: CTATGAGGACCTCATGCGCT; PIK3CD_pos1 GGGCAGTCCTGCAGAAGGAC; PIK3CD_pos2 GTATGAGCACGAGAAGGACC; PIK3CD_pos3 GGAGCTGTATGAGCACGAGA; PIK3CD_pos4 AGCACGAGAAGGACCTGGTG; PIK3CD_pos5 GAACGCCGACGAGCGGATGA; PIK3CD_pos6 TGGAGCAGCTGAGCTCAGGC; PIK3CD_neg1 GATGGAGGAGGAATGGACCA; PIK3CD_neg2 GGATGAATGGGACACGCTCG; PIK3CD_neg3 TGGAAGGTGAAGCTCTCCTG or having at least 80%, 82%, 85%, 87%, 88%, 89%, 90%, 92%, 94%, 95%, 96%, 97%, 98% or 99% nucleic acid sequence identity thereto.

### BRIEF DESCRIPTION OF THE FIGURES

FIGS. 1A-1B provide genes modified in a library of T cells infected with a library of sgRNA, the sites of modification and the sgRNAs, using a 3.0 cutoff. A) Genes 1-36 and sites modified and B) sgRNAs.

FIGS. 2A-2B provide genes modified in a library of T cells infected with a library of sgRNA, the sites of modification and the sgRNAs, using a 2.0 cutoff. A) Genes 37-135 sites modified), and B) sgRNAs.

FIGS. 3A-3B provide genes modified in a library of T cells infected with a library of sgRNA, the sites of modification and the sgRNAs, using a 1.0 cutoff. A) Genes 136-367 sites modified, and B) sgRNAs.

FIGS. 4A-4B provide genes modified in a library of T cells infected with a library of sgRNA, the sites of modification and the sgRNAs, using a 0.5 cutoff. A) Genes 368-743 site modified, and B) sgRNAs.

FIGS. 5A-5D. Base Editor Screens in Primary Human T cells. A) Schematic of the pool screening/sorting approach for base editing in primary T cells. B) Evidence that guides targeting the markers which were sorted for are predominantly depleted—for the markers TNFa, IFNg, CD25 (coding gene IL2RA) and PD1 (coding gene PDCD1)→demonstrates expected effects/controls of the screen. C) Filtering for guides that encode a knockout (splice site edit or new stop codon introduction) demonstrates that positive and negative regulators of cytokine production are enriched/depleted as expected. D) Base editing allows alteration of function that is fundamentally different from knockout—allowing increased cytokine production from modification of positive regulators and vice-versa (i.e., genes which would usually be delirious to cytokine production when knocked out can now be edited to enhance cytokine production and vice versa).

FIGS. 6A-6C. Lentiviral delivery of Base Editors in primary human T cells. A) Flow cytometry with annotated mutations for CD3, 5, 7 genes (3 sgRNAs per gene). B) Sanger sequencing data showing underlying highly efficient mutations (which can or cannot result in downregulated protein as seen in A). C) quantification of flow cytometry results for all sgRNAs

FIGS. 7A-7B. Extended Validation of selected perturbations identified by the screen. A) Similar to 3D but for membrane expressed activation markers of T cells. B) Response of perturbed T cells under different strengths of stimulation.

FIG. 8. Full panel live imaging data over time for all T-cell to cancer cell ratios and all perturbations.

FIGS. 9A-9G. Validation of selected perturbations identified by the screen. A) Schematic: Mutations were introduced by Base Editor mRNA +sgRNA electroporation in an arrayed format. B) Validation of edits by NGS. C) Flow cytometry plots of selected perturbations showing up or downregulation of target markers, as identified by the screen. D) Quantification of flow cytometry results, presented as Log2Fold Change over control (AAVS1). In red: positive regulators as predicted by the screen. In blue: negative regulators as predicted by the screen. E) Quantification of a broader panel of secreted cytokines (data in ~2 weeks).

FIGS. 10A-10B provide genes modified in a library of T cells infected with a library of sgRNA, the sites of modification (A) and the sgRNAs (B), gated on a screen log-fold-change of >1.0 or <−3.0 and hits that behave the opposite from a knockout in the screen (i.e., the sign of the LFC is opposite) and have LFC of >0.4.

FIG. 11 provides genes 1-47 modified in a library of T cells infected with a library of sgRNA, and the sites of modification for those genes.

FIG. 12 provides the sgRNAs for genes 1-47 of FIG. 11.

FIG. 13 provides a schematic for therapeutic engineering of human immune cells.

FIG. 14 provides a schematic for forward genetic discovery of functional alleles with base editors.

FIG. 15 provides a schematic for base editing enabled multiplexed KO in cells, such as T cells.

FIG. 16 provides data showing a scalable base editing system in human T cells.

FIG. 17 provides a schematic of functional protein engineering in primary cells.

FIG. 18 provides several T cell genes.

FIG. 19 demonstrates that mutation effects are reproducible between human donors.

FIG. 20 discloses the power of the disclosed system in the context of discovering mutations that affect protein expression.

FIG. 21 discloses the power of the disclosed system in the context of being able to demonstrate distinct effects of mutations in the same genes.

FIGS. 22-24 show that scanning mutations reveal protein residues and domains.

FIG. 25 shows high resolution base editing with NG PAM Cas9.

FIG. 26 further demonstrates NG PAM Cas9 base editor screen (58 genes).

FIGS. 27-29 demonstrate that NG PAM Cas9 screens provide increased resolution.

FIG. 30 provides a 3D structure demonstrating that scanning mutations reveal protein residues and domains.

FIG. 31 provides a schematic for targeted knock-ins of candidate variants.

FIGS. 32 and 33 provide a schematic for CRISPR base editing.

FIG. 34 demonstrates that mutation effects are reproducible between human donors.

FIG. 35 provides graphs showing in vitro cancer killing with different target antigens.

FIG. 36 further demonstrates NG PAM Cas9 base editor screen (54 genes).
Schmidt et al. Nature. Base-editing mutagenesis maps alleles to tune human T cell functions. Published online 13 Dec. 2023. doi.org/10.1038/s41586-023-06835-6, incorporated herein by reference.

### DETAILED DESCRIPTION

Base Editing is a method that, guided by CRISPR-Cas9, introduces specific A to G or C to T mutations in the genomic DNA without double-strand breaks. This method has traditionally been used to correct specific pathologic mutations with increased on-target fidelity and minimized translocation risk. More recently, Base Editing was used to introduce loss of-function mutations in primary human T cells to create functional knockouts. While Cytidine base editors (CBE) can directly introduce stop codons by converting amino acid coding triplets into TGA, TAA or TAG, ABE and CBE both can cause functional knockouts by altering splice sites (to induce frame shift) or removing start codons. These approaches have successfully been used to introduce multiplexed knockouts in primary human T cells. It was hypothesized that Base Editing could be used for functional interrogation of proteins at amino acid level resolution.

For example, one or more target regions in one or more selected gene are identified (e.g., identified by an editing window spanning a portion of the sgRNA length including bases directly upstream of the 5′ end of the sgRNA) that can be modified to alter gene function (e.g., to fine tune in a positive or negative manner). In one embodiment, the selected genes play roles in T cell biology or are members of families of genes involved in T cell/lymphocyte biology. In one embodiment, all available sgRNAs with an NGG PAM sequence with the editing window being (at least in part) in the coding regions of these genes were prepared, thus tiling the entire coding region of 386 genes. Since this was not a traditional CRISPR knockout screen, but a Base Editing screen, the chosen approach allowed us to mutate specific parts of these genes without causing frameshift mutations. Although the 386 genes that were modified are involved in T cell function/biology, at least some of the gene have roles in other immune cells, e.g., lymphocytes. Thus, the identified sgRNAs can be employed to modify any cell that expresses the target gene or to identify functional or targetable regions within the respective protein independent of its cellular expression pattern. More broadly, the screens define a targetable region in these genes other than that for the specific sgRNAs employed because of the absence of control for introduced amino acid changes that occur at a defined pattern (within the editing window, A becomes G or C becomes T). Thus, sgRNAs were used to define gene regions that, when altered, influence gene function (decrease or enhance).

Further, given the broad variety of sgRNAs, the information provided by the screen allows for the fine tuning of the function of respective gene product (in comparison to traditional knockout where it is an all-or-nothing mutation). Some of the hotspot regions that were identified may be used to introduce the opposite effect a traditional knockout would have (e.g., enhance protein function where knockout). Furthermore, this approach allows targeting of specific functions within the overall functionality the respective protein.

The sgRNAs may be employed to modify gene/protein function, for example, alter but not eliminate the function(s), in cells including but not limited to Pan T cells, CD4 T cells, CD8 T cells, regulatory T cells, NK cells, B cells or other lymphoid cells to also alter their function in a therapeutic setting (enhance activity or decrease specific activity). The identified protein modification of region allowing for modified protein functionality could be used in any context this gene or protein might be expressed, including other cell types or as a synthetic approach. For example, cells not naturally expressing the protein of interest could be modified by expression of an identified mutant of that protein to alter cellular functions. In another example, the modified protein could be incorporated in other synthetic therapies, e.g., anti-cancer or anti-autoimmune T cells.

In one embodiment, using a cutoff of 3.0, 36 genes having modifications were identified, e.g., those modifications resulted in a stronger effect/alteration in function. The regions that were modified may be further altered, e.g., substituting one or more amino acids, which may further enhance or may decrease the activity of the resulting variant gene product.

### Exemplary Screening Methods

Previous CRISPR-enabled screening has primarily focused on whole gene inhibition/knockout or activation, whereas a high-resolution method can identify functionally distinct regions within a gene, which may be of therapeutic relevance. When combined with large scale phenotypic screening approaches, the effects of amino acid level disruption across hundreds of genes involved in T cell activity can rapidly be determined. This high throughput examination enables quick identification of functionally distinct regions—regions that performed better or orthogonally to inhibition/activation of the entire gene—that could also be used for therapeutic applications. For example, genes and coding regions may be identified more finely tuned T cell responses; sgRNA may be identified for base editing mediated knockout, alleviating many concerns with standard CRISPR knockout; or loss/gain of function mutations into specific function regions of a protein may be identified, allowing dissection of different functions of a single protein.

To optimize this technology for screening in primary human T cells, the efficiency of a variety of base editors on specific genes was tested. Lentiviral transfer plasmids of the second generation were modified to enhance the titer. With these constructs, highly efficient edits were produced with base editor and sgRNA delivered on separated viruses. By adding a combination of blasticidin and puromycin to this constructs, successfully transduced cells were enriched, yielding a pure population of edited cells. Having established the delivery and functionality of the base editor machinery in primary human T cell for pooled screening (it was found that the lentiviral approach was superior to a hybrid sgRNA lentivirus approach+base editor mRNA), an sgRNA library was prepared for the coding region of 385 genes involved in T cell activity. T cells were sorted for their capability to produce the cytokines TNFa and IFNg as well as the activation induced surface markers CD25 and PD1. T cells were also subjected to repeated stimulation to test which sgRNA mediated mutations provide a proliferative advantage compared to control cells. sgRNAs and groups of sgRNAs which mapped to distinct regions within target genes and caused gain-or-loss of T cell activity were identified. Notably, these effects frequently exceeded the effects of knockout/activation or caused the opposite effect of knockout/activation—highlighting the power of base editing to introduction functional changes to genes which are otherwise not possible. sgRNAs were found that mediate negative effects as well as sgRNAs mediating positive effects within the same gene, using a single editing method to cause opposite effects. Consequently, the findings were validated by assessing T cell activation in arrayed format with single sgRNAs targeting representative regions within a selected set of genes. It was also shown that some of the introduced perturbations increased the cancer killing potency of T cells in vitro.

In summary, a highly efficient method to perform pooled Base Editing screens in primary human T cells was identified. This includes but is not limited to ABE, CBE and with different types of Cas9 (Cas9 with NGG PAM, SpG Cas9 with NG PAM, or NG Cas9 with NG PAM). This approach was used to identify genomic regions (at the amino acid level) in coding regions which can be targeted to alter T cell functionality in multiple contexts. Thus, methods and compositions to generate base edited T cells with improved functionality that could enhance immunotherapies are provided. The functionality includes direct responses to T cell activation as well as the integration of environmental factors. A set of screens identified causal relationships between about 117,000 mutation sites in the entire coding regions of selected genes and their effect on T cell activation and function. Thus, the method provides a discovery platform to identify therapeutic targets.

In one embodiment, a CBE is employed. In one embodiment, the CBE is a first-generation base-editor (CBE1), one fusing a rat-derived cytosine deaminase Apolipoprotein B MRNA Editing Enzyme Catalytic Subunit 1 (APOBEC1) to the amino terminus of catalytically deficient, or “dead”, Cas9 (dCas9). In a narrow window of the non-targeted strand, CBE1 deaminates cytosine to uracil. Uracil is then recognized by cell replication machinery as a thymine, resulting in a C-G to T-A transition. In one embodiment, the base editor is a second-generation cytosine base-editor (CBE2), e.g., one prepared by fusing an uracil DNA glycosylase inhibitor (LTGI) to the C-terminus of BE1, inhibiting the activity ofUDG. BE3 was developed by restoring histidine at position 840 (H840, HNH catalytic domain) in dCas9 to generate a base-editor that uses Cas9 nickase (nCas9). This variant induces a nick in the G-containing strand of the U-G intermediate (non-edited DNA strand) to bias cellular repair of the intermediate towards a U-A outcome, further converted to T-A during DNA replication. In one embodiment, the base editor is a fourth-generation cytosine base-editor (CBE4), e.g., generated by fusing an additional copy of UGI to the N terminus of nCas9 with an optimized 27 bp linker. YEE-BE3 was developed by screening several mutations previously reported to modulate the catalytic activity of cytosine deaminases in the APOBEC family to generate an improved rAPOBEC1 with a narrower editing window and reduced “bystander editing” compared to CBE3. Gam, a DNA-binding protein from bacteriophage Mui, can form a complex with free-ends of DBSs, thus preventing NHEJ-mediated repair and reducing indel formation. These changes resulted in BE4-Gam, which is characterized by higher base-editing efficiency, increased product purity, and decreased indel frequency. Two nuclear localization signals (NLS) were added to nCas9 and after codon-optimization and ancestral sequence reconstruction on APOBEC, yielding BE4max and ancBE4max. Another base-editing system, Target-AID (activation-induced cytidine deaminase), was developed and composed of nCas9, Petromnyzon marinns cytidine deaminase 1 (pmCDA1), which is similar to rAPOBEC1 in structure and function, The use of alternative cytosine deaminase enzymes yields base editors with alternative sequence motif preference and the ability to efficiently edit methylated cytosines. Most recently, Liu and colleagues used phase assisted continuous evolution (PACE) to evolve CBEs and generate evoAPOBEC1-BE4max, which can efficiently edit cytosine in G/C sequences (a disfavored context for wild-type APOBEC1 deaminase) and evoFERNY-BE4max, a smaller deaminase that edits efficiently in all tested sequence contexts.

Base-editors incorporating different CRISPR-associated nuclease enzymes are also envisioned for use in the method. CBEs based on SpCas9 are limited by their G/C-rich PAM sequence. In order to expand the scope of base-editing, Li et al. generated a Cpf1-based cytosine deaminase base-editor by fusing catalytically inactive LbCpf1 (dLbCpf1) or dAsCpf1 with rAPOBEC1 and UGI (creating dLbCpf1-BE0 and dAsCpf1-BE0). A variety of engineered Cas9 variants with altered PAM sequences and improved cleavage specificity have been developed and may allow for further expansion of the targeting scope of CRISPR-base-editing reagents.

Adenine base-editors induce A to G conversions. The ABE-dCas9 fusion binds to a target DNA sequence in a guide RNA-programmed manner, and the deoxyadenosine deaminase domain catalyzes an adenine to inosine transition. In the context of DNA replication, inosine is interpreted as guanine, and the original A-T base pair may be replaced with a G-C base pair at the target site. Escherichia coli tRNA adenosine deaminase, TadA (ecTadA) converts adenine to inosine in the single-stranded anticodon loop of tRNA^{ARG}, and shares sequence similarity with the APOBEC family. The first-generation adenine base-editors (ABE1.2) was generated by fusing the evolved TadA variant (TadA*) to the N-terminus of nCas9 through XTEN (a 16 amino acid linked used in BE3), with the C terminal of nCas9 fused with a nuclear localization signal (TadA*-XTEN-nCas9-NLS). In comparison with cytosine base-editing, adenine base-editing by ABE yields a much cleaner product that has virtually no indels, and there are no reports of significant off-target (A-to-non-G) edits to date.

To improve ABEs, a single-chain heterodimer was prepared comprised of a wild-type non-catalytic TadA monomer and evolved ecTadA monomer (TadA-TadA*). To improve editing efficiency, further optimization of ABE was performed. Extensive PACE and protein engineering resulted in seventh generation ABEs (ABE7.10), which converted target A-T to G-C efficiently (~50%) in human cells. Only about one-quarter of pathogenic transition mutations encompass an appropriately located NGG PAM site that facilitates SpCas9-mediated base-editing. Unlike CBEs, which have proven to be broadly customizable with many Cas orthologs, ABEs have shown limited compatibility with Cas9 of any origin other than SpCas9. Some homologs such as SaCas9 and circularly permuted Cas9 (CP-Cas9) have been adapted. Phage-assisted continuous and non-continuous evolution (PACE and PANCE) methods were used to enhance the catalytic rate of the deoxyadenosine deaminase enzyme by 590-fold compared to that of ABE7. 10. The next generation of ABEs, designated ABE8e, shows greatly enhanced activity and compatibility with diverse Cas9 homologs. As expected, the targeting scope of ABE8e also increased off-target RNA and DNA editing.

Exemplary base editors useful in the screening methods include but are not limited to ABE8e, BE1, BE2, HF2-BE2, BE3, HF-BE3, YE1-BE3, EE-B3, YEE-BE3, VQR-BE3, EQR-BE3, VRER-BE3, SaKKHBE3, FNLS-BE3, RA-BE3, A3A-BE3, eA3A-HF1-BE3-2xUGI, eA3A-Hypa-BE3-2xUGI, hA3A-BE3, hA3B-BE3, hA3G-BE3, hAID-BE3, SaCas9-BE3, xCas9-BE3, ScCas9-BE3, SniperCas9-BE3, iSpyMac-BE3, Target-AID, Target-AID-NG, CRISPR-X, TAM, BE-PLUS, BE4, BE4-Gam, BE4-Max, AncBE4-Max, SaCas9-BE4, SaCas9-BE4-Gam, evoBE4max, evoFERNY-BE4max, Cas12a-BE, ABE7.8/9/10, xCas9-ABE7.10, VQR-ABE, Sa(KKH)-ABE, ABEmax, ABE7.10max, ABE8e, PE1, PE2, or PE3.

In other screens, small molecule drugs targeting one of the identified functional regions can be identified that allow for enhanced or decreased immune activity (in a variety of immune related pathologies including cancer, autoimmunity, and the like).

The use of the method may provide for identification and targeting of protein sub-functions (e.g., higher resolution than overall loss- or gain-of function methods), tools to identify targetable structures of proteins, and identification of hits and functional regulators for exploitation as therapeutic targets.

The use of a method for high throughput base editing screens in primary human T cells using lentivirus provides for an approach for high throughput screening of protein/gene domains at amino acid level, in primary human T cells and may result in a large set of previously uncharacterized targets that alter T cell function, base edited T cells with enhanced functionality and the ability to dissect different (including opposing) functional regions within a protein and exploitation for therapeutic or diagnostic purposes.

The methods and resulting composition provide for rapid discovery, including discovery of variants or mutations that affect cell functions in patients, and therapeutic applicability of targets for immune cell therapies, highly multiplexable and seamless integration with other gain- or loss-of function strategies, loss and gain of protein (sub-) functions, e.g., using a single editing method, multiplexable and/or within a single protein, identification of sgRNAs to mediate functional base editor knockouts of desired genes, and discovery and targeting of protein (e.g. domain or amino acid specific) subfunctions.

### Exemplary Cells for Modification

In one embodiment, the cells that are modified with sgRNAs or the genome of which is modified to have the modified sites described herein or expresses a protein have one or more of the modifications disclosed herein, are immune cells. In one embodiment, the cells are T cells. In one embodiment, the cells are CD4+ cells. In one embodiment, the cells are CD8+ cells. In one embodiment, the cells are CAR-T cells. In one embodiment, the cells are naive T cells, stem cell memory cells, T _{SCM}; T Central Memory cells, T _{CM}; T effector memory cells, T _{EM}; or T effector cells, T _{EFF}. In one embodiment, the cells are Th (T helper)1, Th2, Th9, Th17, Th22, Treg (regulatory T cells), or Tfh (follicular helper T cells). In one embodiment, the cells are regulatory T cells, NK cells, or B cells.

### Exemplary Therapeutic Uses of Modified Cells

Ex-vivo or in-vivo genome edited immune cells can be employed for therapeutic purposes. In addition, synthetic constructs that alter immune cell function (e.g., by incorporating or using functional proteins or protein domains that are identified by the screening method) may be employed for therapeutic purposes.

Immune cells modified as described herein may be employed in a method to prevent, inhibit or treat an autoimmune disease. In one embodiment, cells of a mammal may be obtained and modified as described herein and reintroduced to the mammal to, for example, suppress an immune function in the mammal, thereby alleviating one or more symptoms of the autoimmune disease. Autoimmune diseases within the scope of this disclosure include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). In one embodiment, the autoimmune disease is multiple sclerosis (MS), systemic sclerosis (SSc), type 1 diabetes (T1D), Grave's disease (GD), systemic lupus erythematosus (SLE), aplastic anemia (AA), or vitiligo.

Immune cells modified as described herein may be employed in a method to prevent, inhibit or treat cancer. In one embodiment, cells of a mammal may be obtained and modified as described herein and reintroduced to the mammal to, for example, augment an immune function in the mammal, thereby alleviating one or more symptoms of the cancer. Cancers within the scope of this disclosure include but are not limited to carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others.

### Definitions

The term “guide RNA” as used herein refers to either a single guide RNA (sgRNA) or a crRNA (spacer). In some cases, the at least one sgRNA has a sequence with at least 95% sequence identity to any of guide RNAs shown in the Figures. In some cases, at least sgRNA has a sequence such as any of the guide RNAs in the Figures, or a nucleotide sequence with at least 80%, 82%, 84%, 85%, 87%, 89%, 90%, 92%, 94%, 95%, 97%, 98% or 99% nucleic acid sequence identity thereto, or a combination thereof. In some cases, cells can be incubated with one or two or more sgRNAs described herein.

A “vector” or “delivery” vehicle refers to a macromolecule or association of macromolecules that comprises or associates with a polynucleotide or polypeptide, and which can be used to mediate delivery of the polynucleotide or polypeptide to a cell or intercellular space, either in vitro or in vivo. Illustrative vectors include, for example, plasmids, viral vectors, liposomes, nanoparticles, or microparticles and other delivery vehicles. In one embodiment, a polynucleotide to be delivered, sometimes referred to as a “target polynucleotide” or “transgene,” may comprise a coding sequence of interest in gene therapy (such as a gene encoding a protein of therapeutic interest), a coding sequence of interest and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

A “transcriptional regulatory sequence” refers to a genomic region that controls the transcription of a gene or coding sequence to which it is operably linked. Transcriptional regulatory sequences of use generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators) and may be comprised of sequence-specific termination sequences or sequence-non-specific terminators or both. A variety of such terminator sequences are known in the art; and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. For example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use generally include at least one transcriptional promoter and may also include one or more enhancers and/or terminators of transcription.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro; and (6) aromatic; trp, tyr, phe.

The disclosure also envisions polypeptides with non-conservative substitutions. Non-conservative substitutions entail exchanging a member of one of the classes described above for another.

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s)are outweighed by the therapeutically beneficial effects.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or there above), e.g., between about 100 and 1,000 nucleotides in length (or any integer therebetween), e.g., between about 200 and 500 nucleotides in length.

For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, or 6 bases or less or 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 82%, 85%, 87%, 90%, 92%, 95%, 97% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As used herein, “substantially pure” or “purified” means an object species is the predominant species present (i.e., on a molar basis it is more abundant than any other individual species in the composition), for instance, a substantially purified fraction is a composition wherein the object species comprises at least about 50 percent (on a molar basis) of all macromolecular species present. Generally, a substantially pure composition will comprise more than about 80 percent of all macromolecular species present in the composition, or more than about 85%, about 90%, about 95%, and about 99%. The object species may be purified to essential homogeneity (contaminant species cannot be detected in the composition by conventional detection methods) wherein the composition consists essentially of a single macromolecular species.

### Preparation of Expression Cassettes

To prepare expression cassettes having sequences encoding a base editor and/or sgRNAs, e.g., disclosed herein (e.g, the Figures), or both, for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotropic virus. Such promoters include the CMV promoter, as well as the SV40 late promoter and retroviral LTRs (long terminal repeat elements), although many other promoter elements well known to the art may be employed, e.g., the MMTV, RSV, MLV or HIV LTR. In one embodiment, expression is inducible.

In one embodiment, a tissue-specific promoter (or enhancer) is employed,

Other elements functional in the host cells, such as introns, enhancers, polyadenylation sequences and the like, may also be a part of the recombinant DNA. Such elements may or may not be necessary for the function of the DNA but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter 30 genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian cells, such as immune cells, by transfection with the DNA or the corresponding RNA, or infection with a virus having an expression vector comprising the recombinant DNA, by any procedure useful for the introduction into a particular cell, e.g., physical or biological methods, to yield a transformed (transgenic) cell having the recombinant DNA so that the DNA sequence of interest is expressed by the host cell. In one embodiment, the recombinant DNA is stably integrated into the genome of the cell.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic cells, such as mammalian, e.g., human cells. Other viral vectors can be derived from poxviruses, e.g., vaccinia viruses, herpes viruses, adenoviruses, adeno-associated viruses, baculoviruses, and the like.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

### Vectors or Vehicles for Delivery

Delivery vectors or vehicles include, for example, viral vectors, microparticles, nanoparticles, liposomes and other lipid-containing complexes, and other macromolecular complexes capable of mediating delivery of a gene, sgRNA or a protein to a host cell, e.g., a gene to provide for recombinant expression of a polypeptide encoded by the gene. Vectors or vehicles can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector or have taken up protein delivered by a vehicle. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Vectors or vehicles within the scope of the disclosure include, but are not limited to, isolated nucleic acid, e.g., plasmid-based vectors which may be extra-chromosomally maintained, and viral vectors, e.g., recombinant adenovirus, retrovirus, lentivirus, herpesvirus, poxvirus, papilloma virus, or adeno-associated virus, including viral and non-viral vectors, or proteins which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Vectors or vehicles may be administered via any route including, but not limited to, intramuscular, buccal, rectal, intravenous or intracoronary administration, and transfer to cells may be enhanced using electroporation and/or iontophoresis. In one embodiment, vectors are locally administered.

In one embodiment, an isolated polynucleotide or vector having that polynucleotide, encoding a polypeptide or fusion protein that has substantial identity, e.g., at least 80% or more, e.g., 85%, 87%, 90%, 92%, 95%, 97%, 98%, 99% and up to 100%, amino acid sequence identity to a protein encoded by one of the genes disclosed herein, or a portion thereof, is envisioned.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Plasmid DNA may be delivered to cells as part of a macromolecular complex, e.g., a liposome or DNA-protein complex, and delivery may be enhanced using techniques including electroporation.

### Exemplary Gene Products for Modification

Once modified gene products with a desirable activity are identified, the genome of other cells may be modified so that the modified gene product is expressed in those cells. For example, modifications of any of the gene products disclosed herein including those exemplified below, that result in altered activity, e.g., in immune cells, may be prepared and employed in methods including methods of treatment.

In one embodiment, a modification is prepared in a gene encoding T-cell surface glycoprotein CD3 zeta chain isoform 1 precursor (Homo sapiens), for example, one NCBI Reference Sequence: NP_932170.1:

| | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding actin, cytoplasmic 1 (Homo sapiens), for example, one having NCBI Reference Sequence: NP_001092.1

| | |
|---|---|
| 1 | MDDDIAALVV DNGSGMCKAG FAGDDAPRAV FPSIVGRPRH QGVMVGMGQK DSYVGDEAQS |
| 61 | KRGILTLKYP IEHGIVINWD DMEKIWHHTF YNELRVAPEE HPVLLTEAPL NPKANREKMT |
| 121 | QIMFETFNTP AMYVAIQAVL SLYASGRTTG IVMDSGDGVT HTVPIYEGYA LPHAILRLDL |
| 181 | AGRDLTDYLM KILTERGYSF TTTAEREIVR DIKEKLCYVA LDFEQEMATA ASSSSLEKSY |
| 241 | ELPDGQVITI GNERFRCPEA LFQPSFLGME SCGIHETTFN SIMKCDVDIR KDLYANTVLS |
| 301 | GGTTMYPGIA DRMQKEITAL APSTMKIKII APPERKYSVW IGGSILASLS TFQQMWISKQ |
| 361 | EYDESGPSIV HRKCF, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding interleukin-2 receptor subunit alpha isoform 1 precursor (Homo sapiens), such as one having NCBI Reference Sequence: NP_000408.1:

| | |
|---|---|
| 1 | MDSYLLMWGL LTFIMVPGCQ AELCDDDPPE IPHATFKAMA YKEGTMLNCE CKRGFRRIKS |
| 61 | GSLYMLCTGN SSHSSWDNQC QCTSSATRNT TKQVTPQPEE QKERKTTEMQ SPMQPVDQAS |
| 121 | LPGHCREPPP WENEATERIY HFVVGQMVYY QCVQGYRALH RGPAESVCKM THGKTRWTQP |
| 181 | QLICTGEMET SQFPGEEKPQ ASPEGRPESE TSCLVTTTDF QIQTEMAATM ETSIFTTEYQ |
| 241 | VAVAGCVFLL ISVLLLSGLT WQRRQRKSRR TI |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding T-cell surface glycoprotein CD3 delta chain isoform A precursor (Homo sapiens), Such as one having NCBI Reference Sequence: NP_000723.1:

| | |
|---|---|
| 1 | MEHSTFLSGL VLATLLSQVS PFKIPIEELE DRVFVNCNTS ITWVEGTVGT LLSDITRLDL |
| 61 | GKRILDPRGI YRCNGTDIYK DKESTVQVHY RMCQSCVELD PATVAGIIVT DVIATLLLAL |
| 121 | GVFCFAGHET GRLSGAADTQ ALLRNDQVYQ PLRDRDDAQY SHLGGNWARN K, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding T-cell surface glycoprotein CD3 epsilon chain precursor (Homo sapiens). For example, one having NCBI Reference Sequence: NP_000724.1:

| | |
|---|---|
| 1 | MQSGTHWRVL GLCLLSVGVW GQDGNEEMGG ITQTPYKVSI SGTTVILTCP QYPGSEILWQ |
| 61 | HNDKNIGGDE DDKNIGSDED HLSLKEFSEL EQSGYYVCYP RGSKPEDANF YLYLRARVCE |
| 121 | NCMEMDVMSV ATIVIVDICI TGGLLLLVYY WSKNRKAKAK PVTRGAGAGG RQRGQNKERP |
| 181 | PPVPNPDYEP IRKGQRDLYS GLNQRRI, |

(SEQ ID NO:), a different isoform of the protein, or a polypeptide having at east 80%, 82%, 85%, 86%, 88%, 90%, 92%, 94%, 95%, 97%, 98% or 99% amino acid sequence identity thereto.

In one embodiment, a modification such as one in a gene encoding T-cell surface glycoprotein CD3 gamma chain precursor (Homo sapiens) having NCBI Reference Sequence: NP_000064.1:

| | |
|---|---|
| 1 | MEQGKGLAVL ILAIILLQGT LAQSIKGNHL VKVYDYQEDG SVLLTCDAEA KNITWFKDGK |
| 61 | MIGFLTEDKK KWNLGSNAKD PRGMYQCKGS QNKSKPLQVY YRMCQNCIEL NAATISGFLF |
| 121 | AEIVSIFVLA VGVYFIAGQD GVRQSRASDK QTLLPNDQLY QPLKDREDDQ YSHLQGNQLR |
| 181 | RN, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding T-cell surface glycoprotein CD5 isoform 1 precursor (Homo sapiens). For example, one having NCBI Reference Sequence: NP_055022.2:

| | |
|---|---|
| 1 | MPMGSLQPLA TLYLLGMLVA SCLGRLSWYD PDFQARLTRS NSKCQGQLEV YLKDGWHMVC |
| 61 | SQSWGRSSKQ WEDPSQASKV CQRLNCGVPL SLGPFLVTYT PQSSIICYGQ LGSFSNCSHS |
| 121 | RNDMCHSLGL TCLEPQKTTP PTTRPPPTTT PEPTAPPRLQ LVAQSGGQHC AGVVEFYSGS |
| 181 | LGGTISYEAQ DKTQDLENFL CNNLQCGSFL KHLPETEAGR AQDPGEPREH QPLPIQWKIQ |
| 241 | NSSCTSLEHC FRKIKPQKSG RVLALLCSGF QPKVQSRLVG GSSICEGTVE VRQGAQWAAL |
| 301 | CDSSSARSSL RWEEVCREQQ CGSVNSYRVL DAGDPTSRGL FCPHQKLSQC HELWERNSYC |
| 361 | KKVFVTCQDP NPAGLAAGTV ASIILALVLL VVLLVVCGPL AYKKLVKKER QKKQRQWIGP |
| 421 | TGMNQNMSFH RNHTATVRSH AENPTASHVD NEYSQPPRNS HLSAYPALEG ALHRSSMQPD |
| 481 | NSSDSDYDLH GAQRL, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding C-Maf-inducing protein isoform C-Mip (Homo sapiens). For instance, one having NCBI Reference Sequence: NP_938204.2

| | |
|---|---|
| 1 | MDVTSSSGGG GDPRQIEETK PLLGGDVSAP EGTKMGAVPC RRALLLCNGM RYKLLQEGDI |
| 61 | QVCVIRHPRT FLSKILTSKF LRRWEPHHLT LADNSLASAT PTGYMENSVS YSAIEDVQLL |
| 121 | SWENAPKYCL QLTIPGGTVL LQAANSYLRD QWFHSLQWKK KIYKYKKVLS NPSRWEVVLK |
| 181 | EIRTLVDMAL TSPLQDDSIN QAPLEIVSKL LSENTNLTTQ EHENIIVAIA PLLENNHPPP |
| 241 | DLCEFFCKHC RERPRSMVVI EVFTPVVQRI LKHNMDFGKC PRLRLFTQEY ILALNELNAG |
| 301 | MEVVKKFIQS MHGPTGHCPH PRVLPNLVAV CLAAIYSCYE EFINSRDNSP SLKEIRNGCQ |
| 361 | QPCDRKPTLP LRLLHPSPDL VSQEATLSEA RLKSVVVASS EIHVEVERTS TAKPALTASA |
| 421 | GNDSEPNLID CLMVSPACST MSIELGPQAD RTLGCYVEIL KLLSDYDDWR PSLASLLQPI |
| 481 | PFPKEALAHE KFTKELKYVI QRFAEDPRQE VHSCLLSVRA GKDGWFQLYS PGGVACDDDG |
| 541 | ELFASMVHIL MGSCYKTKKF LLSLAENKLG PCMLLALRGN QTMVEILCLM LEYNIIDNND |
| 601 | TQLQIISTLE STDVGKRMYE QLCDRQRELK ELQRKGGPTR LTLPSKSTDA DLARLLSSGS |
| 661 | FGNLENLSLA FTNVTSACAE HLIKLPSLKQ LNLWSTQFGD AGLRLLSEHL TMLQVLNLCE |
| 721 | TPVTDAGLLA LSSMKSLCSL NMNSTKLSAD TYEDLKAKLP NLKEVDVRYT EAW, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding capping protein-inhibiting regulator of actin dynamics isoform 1 (Homo sapiens) having NCBI Reference Sequence: NP_001380310.1:

| | |
|---|---|
| 1 | MGTRAFSHDS IFIPDGGAES EQTVQAMSQD NILGKVKTLQ QQLGKNIKFG QRSPNAIPMN |
| 61 | KANSGEASLE EDLFLTSPME IVTQQDIVLS DAENKSSDTP SSLSPLNLPG AGSEMEEKVA |
| 121 | PVKPSRPKRH FSSAGTIESV NLDAIPLAIA RLDNSAAKHK LAVKPKKQRV SKKHRRLAQD |
| 181 | PQHEQGGLES RPCLDQNGHP GEDKPTWHEE EPNPLDSEEE RRRQEDYWRE LEAKCKRQKA |
| 241 | EAAEKRRLEE QRLQALERRL WEENRRQELL EEEGEGQEPP LEAERAPREE QQRSLEAPGW |
| 301 | EDAERREREE RERLEAEEER RRLQAQAQAE ERRRLEEDAR LEERRRQEEE EGRCAEELKR |
| 361 | QEEEEAEGWE ELEQQEAEVQ GPPEALEETG EGRRGAEEED LGEEEEEGQA HLEDWRGQLS |
| 421 | ELLNDFEERL EDQERLKPEG QREHSEEPGI CEEQNPEAER RREQQGRSGD FQGADRPGPE |
| 481 | EKREEGDTEP LLKQEGPVEA AQPPVERKEA AALEQGRKVE ELRWQEVDER QTMPRPYTFQ |
| 541 | VSSGGKQILF PKVNLSPVTP AKDTGLTAAP QEPKAPKASP VQHALPSSLS VPHTAILVTG |
| 601 | AQLCGPAVNL SQIKDTACKS LLGLEEKKHA EAPAGENPPR GPGDARAGSG KAKPRQESPS |
| 661 | SASALAEWAS IRSRILKNAE SDPRSSERDQ LRPGDESTPR GRCDSRGNQR KTPPVNAKFS |
| 721 | IMPAWQKFSD GGTETSKQST EAESIRKRPM LGPSEETAPQ PPPAGVRELG KGPEKSEMHR |
| 781 | EPADTTEGCK FAKDLPSELV PSLPYPPQKV VAHTEFTTSS DSETANGIAK PDPVMPGGEE |
| 841 | KASPFGIKLR RTNYSLRFNC DQQAEQKKKK RHSSTGDSAD AGPPAAGSAR GEKEMEGVAL |
| 901 | KHGPSLPQER KQAPSTRRDS AEPSSSRSVP VAHPGPPPAS SQTPAPEHDK AANKMPLAQK |
| 961 | PALAPKPTSQ TPPASPLSKL SRPYLVELLS RRAGRPDPEP SEPSKEDQES SDRRPPSPPG |
| 1021 | PEERKGQKRD EEEEATERKP ASPPLPATQQ EKPSQTPEAG RKEKPMLQSR HSLDGSKLTE |
| 1081 | KVETAQPLWI TLALQKQKGF REQQATREER KQAREAKQAE KLSKENVSVS VQPGSSSVSR |
| 1141 | AGSLHKSTAL PEEKRPETAV SRLERREQLK KANTLPTSVT VEISDSAPPA PLVKEVTKRF |
| 1201 | STPDAAPVST EPAWLALAKR KAKAWSDCPQ IIK, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding elongin-B isoform a (Homo sapiens), including one having NCBI Reference Sequence: NP_009039.1:

| | |
|---|---|
| 1 | MDVFLMIRRH KTTIFTDAKE SSTVFELKRI VEGILKRPPD EQRLYKDDQL LDDGKTLGEC |
| 61 | GFTSQTARPQ APATVGLAFR ADDTFEALCI EPFSSPPELP DVMKPQDSGS SANEQAVQ, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one described in a gene encoding trans-acting T-cell-specific transcription factor GATA-3 isoform 1 (Homo sapiens), including one having NCBI Reference Sequence: NP_001002295.1:

| | |
|---|---|
| 1 | MEVTADQPRW VSHHHPAVLN GQHPDTHHPG LSHSYMDAAQ YPLPEEVDVL FNIDGQGNHV |
| 61 | PPYYGNSVRA TVQRYPPTHH GSQVCRPPLL HGSLPWLDGG KALGSHHTAS PWNLSPFSKT |
| 121 | SIHHGSPGPL SVYPPASSSS LSGGHASPHL FTFPPTPPKD VSPDPSLSTP GSAGSARQDE |
| 181 | KECLKYQVPL PDSMKLESSH SRGSMTALGG ASSSTHHPIT TYPPYVPEYS SGLFPPSSLL |
| 241 | GGSPTGFGCK SRPKARSSTE GRECVNCGAT STPLWRRDGT GHYLCNACGL YHKMNGQNRP |
| 301 | LIKPKRRLSA ARRAGTSCAN CQTTTTTLWR RNANGDPVCN ACGLYYKLHN INRPLTMKKE |
| 361 | GIQTRNRKMS SKSKKCKKVH DSLEDFPKNS SFNPAALSRH MSSLSHISPF SHSSHMLTTP |
| 421 | TPMHPPSSLS FGPHHPSSMV TAMG, |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding GRB2-related adapter protein 2 isoform 1 (Homo sapiens), such as one having NCBI Reference Sequence: NP_004801.1:

| | |
|---|---|
| 1 | MEAVAKFDFT ASGEDELSFH TGDVLKILSN QEEWFKAELG SQEGYVPKNF IDIQFPKWFH |
| 61 | EGLSRHQAEN LLMGKEVGFF IIRASQSSPG DFSISVRHED DVQHFKVMRD NKGNYFLWTE |
| 121 | KFPSLNKLVD YYRTNSISRQ KQIFLRDRTR EDQGHRGNSL DRRSQGGPHL SGAVGEEIRP |
| 181 | SMNRKLSDHP PTLPLQQHQH QPQPPQYAPA PQQLQQPPQQ RYLQHHHFHQ ERRGGSLDIN |
| 241 | DGHCGTGLGS EMNAALMHRR HTDPVQLQAA GRVRWARALY DFEALEDDEL GFHSGEVVEV |
| 301 | LDSSNPSWWT GRLHNKLGLF PANYVAPMTR |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one described in a gene encoding growth factor receptor-bound protein 2 isoform 1 (Homo sapiens), such as one having NCBI Reference Sequence: NP_002077.1:

| | |
|---|---|
| 1 | MEAIAKYDFK ATADDELSFK RGDILKVLNE ECDQNWYKAE LNGKDGFIPK NYIEMKPHPW |
| 61 | FFGKIPRAKA EEMLSKQRHD GAFLIRESES APGDFSLSVK FGNDVQHFKV LRDGAGKYFL |
| 121 | WVVKFNSLNE LVDYHRSTSV SRNQQIFLRD IEQVPQQPTY VQALFDFDPQ EDGELGFRRG |
| 181 | DFIHVMDNSD PNWWKGACHG QTGMFPRNYV TPVNRNV |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding DNA-binding protein Ikaros isoform 1 (Homo sapiens), such as one having NCBI Reference Sequence: NP_006051.1:

| | |
|---|---|
| 1 | MDADEGQDMS QVSGKESPPV SDTPDEGDEP MPIPEDLSTT SGGQQSSKSD RVVASNVKVE |
| 61 | TQSDEENGRA CEMNGEECAE DLRMLDASGE KMNGSHRDQG SSALSGVGGI RLPNGKLKCD |
| 121 | ICGIICIGPN VLMVHKRSHT GERPFQCNQC GASFTQKGNL LRHIKLHSGE KPFKCHLCNY |
| 181 | ACRRRDALTG HLRTHSVGKP HKCGYCGRSY KQRSSLEEHK ERCHNYLESM GLPGTLYPVI |
| 241 | KEETNHSEMA EDLCKIGSER SLVLDRLASN VAKRKSSMPQ KFLGDKGLSD TPYDSSASYE |
| 301 | KENEMMKSHV MDQAINNAIN YLGAESLRPL VQTPPGGSEV VPVISPMYQL HKPLAEGTPR |
| 361 | SNHSAQDSAV ENLLLLSKAK LVPSEREASP SNSCQDSTDT ESNNEEQRSG LIYLTNHIAP |
| 421 | HARNGLSLKE EHRAYDLLRA ASENSQDALR VVSTSGEQMK VYKCEHCRVL FLDHVMYTIH |
| 481 | MGCHGFRDPF ECNMCGYHSQ DRYEFSSHIT RGEHRFHMS |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding cytokine receptor common subunit gamma precursor (Homo sapiens), such as one having NCBI Reference Sequence: NP_000197.1:

| | |
|---|---|
| 1 | MLKPSLPFTS LLFLQLPLLG VGLNTTILTP NGNEDTTADF FLTTMPTDSL SVSTLPLPEV |
| 61 | QCFVFNVEYM NCTWNSSSEP QPTNLTLHYW YKNSDNDKVQ KCSHYLFSEE ITSGCQLQKK |
| 121 | EIHLYQTFVV QLQDPREPRR QATQMLKLQN LVIPWAPENL TLHKLSESQL ELNWNNRFLN |
| 181 | HCLEHLVQYR TDWDHSWTEQ SVDYRHKFSL PSVDGQKRYT FRVRSRFNPL CGSAQHWSEW |
| 241 | SHPIHWGSNT SKENPFLFAL EAVVISVGSM GLIISLLCVY FWLERTMPRI PTLKNLEDLV |
| 301 | TEYHGNFSAW SGVSKGLAES LQPDYSERLC LVSEIPPKGG ALGEGPGASP CNQHSPYWAP |
| 361 | PCYTLKPET |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding interferon regulatory factor 4 isoform 1 (Homo sapiens), such as one having NCBI Reference Sequence: NP_002451.2:

| | |
|---|---|
| 1 | MNLEGGGRGG EFGMSAVSCG NGKLRQWLID QIDSGKYPGL VWENEEKSIF RIPWKHAGKQ |
| 61 | DYNREEDAAL FKAWALFKGK FREGIDKPDP PTWKTRLRCA LNKSNDFEEL VERSQLDISD |
| 121 | PYKVYRIVPE GAKKGAKQLT LEDPQMSMSH PYTMTTPYPS LPAQQVHNYM MPPLDRSWRD |
| 181 | YVPDQPHPEI PYQCPMTFGP RGHHWQGPAC ENGCQVTGTF YACAPPESQA PGVPTEPSIR |
| 241 | SAEALAFSDC RLHICLYYRE ILVKELTTSS PEGCRISHGH TYDASNLDQV LFPYPEDNGQ |
| 301 | RKNIEKLLSH LERGVVLWMA PDGLYAKRLC QSRIYWDGPL ALCNDRPNKL ERDQTCKLFD |
| 361 | TQQFLSELQA FAHHGRSLPR FQVTLCFGEE FPDPQRQRKL ITAHVEPLLA RQLYYFAQQN |
| 421 | SGHFLRGYDL PEHISNPEDY HRSIRHSSIQ E |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding tyrosine-protein kinase ITK/TSK (Homo sapiens), such as one having NCBI Reference Sequence: NP_005537.3:

| | |
|---|---|
| 1 | MNNFILLEEQ LIKKSQQKRR TSPSNFKVRF FVLTKASLAY FEDRHGKKRT LKGSIELSRI |
| 61 | KCVEIVKSDI SIPCHYKYPF QVVHDNYLLY VFAPDRESRQ RWVLALKEET RNNNSLVPKY |
| 121 | HPNFWMDGKW RCCSQLEKLA TGCAQYDPTK NASKKPLPPT PEDNRRPLWE PEETVVIALY |
| 181 | DYQTNDPQEL ALRRNEEYCL LDSSEIHWWR VQDRNGHEGY VPSSYLVEKS PNNLETYEWY |
| 241 | NKSISRDKAE KLLLDTGKEG AFMVRDSRTA GTYTVSVFTK AVVSENNPCI KHYHIKETND |
| 301 | NPKRYYVAEK YVFDSIPLLI NYHQHNGGGL VTRLRYPVCF GRQKAPVTAG LRYGKWVIDP |
| 361 | SELTFVQEIG SGQFGLVHLG YWLNKDKVAI KTIREGAMSE EDFIEEAEVM MKLSHPKLVQ |
| 421 | LYGVCLEQAP ICLVFEFMEH GCLSDYLRTQ RGLFAAETLL GMCLDVCEGM AYLEEACVIH |
| 481 | RDLAARNCLV GENQVIKVSD FGMTRFVLDD QYTSSTGTKF PVKWASPEVF SFSRYSSKSD |
| 541 | VWSFGVLMWE VFSEGKIPYE NRSNSEVVED ISTGFRLYKP RLASTHVYQI MNHCWKERPE |
| 601 | DRPAFSRLLR QLAEIAESGL |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding tyrosine-protein kinase JAK1 isoform 1 (Homo sapiens), such as one having NCBI Reference Sequence: NP_002218.2:

| | |
|---|---|
| 1 | MQYLNIKEDC NAMAFCAKMR SSKKTEVNLE APEPGVEVIF YLSDREPLRL GSGEYTAEEL |
| 61 | CIRAAQACRI SPLCHNLFAL YDENTKLWYA PNRTITVDDK MSLRLHYRMR FYFTNWHGTN |
| 121 | DNEQSVWRHS PKKQKNGYEK KKIPDATPLL DASSLEYLFA QGQYDLVKCL APIRDPKTEQ |
| 181 | DGHDIENECL GMAVLAISHY AMMKKMQLPE LPKDISYKRY IPETLNKSIR QRNLLTRMRI |
| 241 | NNVFKDFLKE FNNKTICDSS VSTHDLKVKY LATLETLTKH YGAEIFETSM LLISSENEMN |
| 301 | WFHSNDGGNV LYYEVMVTGN LGIQWRHKPN VVSVEKEKNK LKRKKLENKH KKDEEKNKIR |
| 361 | EEWNNFSYFP EITHIVIKES VVSINKQDNK KMELKLSSHE EALSFVSLVD GYFRLTADAH |
| 421 | HYLCTDVAPP LIVHNIQNGC HGPICTEYAI NKLRQEGSEE GMYVLRWSCT DFDNILMTVT |
| 481 | CFEKSEQVQG AQKQFKNFQI EVQKGRYSLH GSDRSFPSLG DLMSHLKKQI LRTDNISFML |
| 541 | KRCCQPKPRE ISNLLVATKK AQEWQPVYPM SQLSFDRILK KDLVQGEHLG RGTRTHIYSG |
| 601 | TLMDYKDDEG TSEEKKIKVI LKVLDPSHRD ISLAFFEAAS MMRQVSHKHI VYLYGVCVRD |
| 661 | VENIMVEEFV EGGPLDLFMH RKSDVLTTPW KFKVAKQLAS ALSYLEDKDL VHGNVCTKNL |
| 721 | LLAREGIDSE CGPFIKLSDP GIPITVLSRQ ECIERIPWIA PECVEDSKNL SVAADKWSFG |
| 781 | TTLWEICYNG EIPLKDKTLI EKERFYESRC RPVTPSCKEL ADLMTRCMNY DPNQRPFFRA |
| 841 | IMRDINKLEE QNPDIVSEKK PATEVDPTHF EKRFLKRIRD LGEGHFGKVE LCRYDPEGDN |
| 901 | TGEQVAVKSL KPESGGNHIA DLKKEIEILR NLYHENIVKY KGICTEDGGN GIKLIMEFLP |
| 961 | SGSLKEYLPK NKNKINLKQQ LKYAVQICKG MDYLGSRQYV HRDLAARNVL VESEHQVKIG |
| 1021 | DFGLTKAIET DKEYYTVKDD RDSPVFWYAP ECLMQSKFYI ASDVWSFGVT LHELLTYCDS |
| 1081 | DSSPMALFLK MIGPTHGQMT VTRLVNTLKE GKRLPCPPNC PDEVYQLMRK CWEFQPSNRT |
| 1141 | SFQNLIEGFE ALLK |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding Krueppel-like factor 2 (Homo sapiens), such as one having NCBI Reference Sequence: NP_057354.1:

| | |
|---|---|
| 1 | MALSEPILPS FSTFASPCRE RGLQERWPRA EPESGGTDDD LNSVLDFILS MGLDGLGAEA |
| 61 | APEPPPPPPP PAFYYPEPGA PPPYSAPAGG LVSELLRPEL DAPLGPALHG RFLLAPPGRL |
| 121 | VKAEPPEADG GGGYGCAPGL TRGPRGLKRE GAPGPAASCM RGPGGRPPPP PDTPPLSPDG |
| 181 | PARLPAPGPR ASFPPPFGGP GFGAPGPGLH YAPPAPPAFG LFDDAAAAAA ALGLAPPAAR |
| 241 | GLLTPPASPL ELLEAKPKRG RRSWPRKRTA THTCSYAGCG KTYTKSSHLK AHLRTHTGEK |
| 301 | PYHCNWDGCG WKFARSDELT RHYRKHTGHR PFQCHLCDRA FSRSDHLALH MKRHM |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding linker for activation of T-cells family member 1 isoform b (Homo sapiens), such as one having NCBI Reference Sequence: NP_001014987.1:

| | |
|---|---|
| 1 | MEEAILVPCV LGLLLLPILA MLMALCVHCH RLPGSYDSTS SDSLYPRGIQ FKRPHTVAPW |
| 61 | PPAYPPVTSY PPLSQPDLLP IPRSPQPLGG SHRTPSSRRD SDGANSVASY ENEEPACEDA |
| 121 | DEDEDDYHNP GYLVVLPDST PATSTAAPSA PALSTPGIRD SAFSMESIDD YVNVPESGES |
| 181 | AEASLDGSRE YVNVSQELHP GAAKTEPAAL SSQEAEEVEE EGAPDYENLQ ELN |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding tyrosine-protein kinase Lck isoform a (Homo sapiens), such as one having NCBI Reference Sequence: NP_005347.3:

| | |
|---|---|
| 1 | MGCGCSSHPE DDWMENIDVC ENCHYPIVPL DGKGTLLIRN GSEVRDPLVT YEGSNPPASP |
| 61 | LQDNLVIALH SYEPSHDGDL GFEKGEQLRI LEQSGEWWKA QSLTTGQEGF IPFNFVAKAN |
| 121 | SLEPEPWFFK NLSRKDAERQ LLAPGNTHGS FLIRESESTA GSFSLSVRDF DQNQGEVVKH |
| 181 | YKIRNLDNGG FYISPRITFP GLHELVRHYT NASDGLCTRL SRPCQTQKPQ KPWWEDEWEV |
| 241 | PRETLKLVER LGAGQFGEVW MGYYNGHTKV AVKSLKQGSM SPDAFLAEAN LMKQLQHQRL |
| 301 | VRLYAVVTQE PIYIITEYME NGSLVDFLKT PSGIKLTINK LLDMAAQIAE GMAFIEERNY |
| 361 | IHRDLRAANI LVSDTLSCKI ADFGLARLIE DNEYTAREGA KFPIKWTAPE AINYGTFTIK |
| 421 | SDVWSFGILL TEIVTHGRIP YPGMTNPEVI QNLERGYRMV RPDNCPEELY QLMRLCWKER |
| 481 | PEDRPTFDYL RSVLEDFFTA TEGQYQPQP |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as in a gene encoding lymphocyte cytosolic protein 2 (Homo sapiens), such as one having NCBI Reference Sequence: NP_005556.1:

| | |
|---|---|
| 1 | MALRNVPFRS EVLGWDPDSL ADYFKKLNYK DCEKAVKKYH IDGARFLNLT ENDIQKFPKL |
| 61 | RVPILSKLSQ EINKNEERRS IFTRKPQVPR FPEETESHEE DNGGWSSFEE DDYESPNDDQ |
| 121 | DGEDDGDYES PNEEEEAPVE DDADYEPPPS NDEEALQNSI LPAKPFPNSN SMYIDRPPSG |
| 181 | KTPQQPPVPP QRPMAALPPP PAGRNHSPLP PPQTNHEEPS RSRNHKTAKL PAPSIDRSTK |
| 241 | PPLDRSLAPF DREPFTLGKK PPFSDKPSIP AGRSLGEHLP KIQKPPLPPT TERHERSSPL |
| 301 | PGKKPPVPKH GWGPDRREND EDDVHQRPLP QPALLPMSSN TFPSRSTKPS PMNPLPSSHM |
| 361 | PGAFSESNSS FPQSASLPPY FSQGPSNRPP IRAEGRNFPL PLPNKPRPPS PAEEENSLNE |
| 421 | EWYVSYITRP EAEAALRKIN QDGTFLVRDS SKKTTTNPYV LMVLYKDKVY NIQIRYQKES |
| 481 | QVYLLGTGLR GKEDFLSVSD IIDYFRKMPL LLIDGKNRGS RYQCTLTHAA GYP |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding mucosa-associated lymphoid tissue lymphoma translocation protein 1 isoform a (Homo sapiens), such as one having NCBI Reference Sequence: NP_006776.1:

| | |
|---|---|
| 1 | MSLLGDPLQA LPPSAAPTGP LLAPPAGATL NRLREPLLRR LSELLDQAPE GRGWRRLAEL |
| 61 | AGSRGRLRLS CLDLEQCSLK VLEPEGSPSL CLLKLMGEKG CTVTELSDFL QAMEHTEVLQ |
| 121 | LLSPPGIKIT VNPESKAVLA GQFVKLCCRA TGHPFVQYQW FKMNKEIPNG NTSELIFNAV |
| 181 | HVKDAGFYVC RVNNNFTFEF SQWSQLDVCD IPESFQRSVD GVSESKLQIC VEPTSQKLMP |
| 241 | GSTLVLQCVA VGSPIPHYQW FKNELPLTHE TKKLYMVPYV DLEHQGTYWC HVYNDRDSQD |
| 301 | SKKVEIIIGR TDEAVECTED ELNNLGHPDN KEQTTDQPLA KDKVALLIGN MNYREHPKLK |
| 361 | APLVDVYELT NLLRQLDFKV VSLLDLTEYE MRNAVDEFLL LLDKGVYGLL YYAGHGYENF |
| 421 | GNSFMVPVDA PNPYRSENCL CVQNILKLMQ EKETGLNVFL LDMCRKRNDY DDTIPILDAL |
| 481 | KVTANIVFGY ATCQGAEAFE IQHSGLANGI FMKFLKDRLL EDKKITVLLD EVAEDMGKCH |
| 541 | LTKGKQALEI RSSLSEKRAL TDPIQGTEYS AESLVRNLQW AKAHELPESM CLKFDCGVQI |
| 601 | QLGFAAEFSN VMIIYTSIVY KPPEIIMCDA YVTDFPLDLD IDPKDANKGT PEETGSYLVS |
| 661 | KDLPKHCLYT RLSSLQKLKE HLVFTVCLSY QYSGLEDTVE DKQEVNVGKP LIAKLDMHRG |
| 721 | LGRKTCFQTC LMSNGPYQSS AATSGGAGHY HSLQDPFHGV YHSHPGNPSN VTPADSCHCS |
| 781 | RTPDAFISSF AHHASCHFSR SNVPVETTDE IPFSFSDRLR ISEK |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding serine/threonine-protein kinase mTOR isoform 1 (Homo sapiens), such as one having NCBI Reference Sequence NP_004949.1:

| | |
|---|---|
| 1 | MLGTGPAAAT TAATTSSNVS VLQQFASGLK SRNEETRAKA AKELQHYVTM ELREMSQEES |
| 61 | TRFYDQLNHH IFELVSSSDA NERKGGILAI ASLIGVEGGN ATRIGRFANY LRNLLPSNDP |
| 121 | VVMEMASKAI GRLAMAGDTF TAEYVEFEVK RALEWLGADR NEGRRHAAVL VLRELAISVP |
| 181 | TFFFQQVQPF FDNIFVAVWD PKQAIREGAV AALRACLILT TQREPKEMQK PQWYRHTFEE |
| 241 | AEKGFDETLA KEKGMNRDDR IHGALLILNE LVRISSMEGE RLREEMEEIT QQQLVHDKYC |
| 301 | KDLMGFGTKP RHITPFTSFQ AVQPQQSNAL VGLLGYSSHQ GLMGFGTSPS PAKSTLVESR |
| 361 | CCRDLMEEKF DQVCQWVLKC RNSKNSLIQM TILNLLPRLA AFRPSAFTDT QYLQDTMNHV |
| 421 | LSCVKKEKER TAAFQALGLL SVAVRSEFKV YLPRVLDIIR AALPPKDFAH KRQKAMQVDA |
| 481 | TVFTCISMLA RAMGPGIQQD IKELLEPMLA VGLSPALTAV LYDLSRQIPQ LKKDIQDGLL |
| 541 | KMLSLVLMHK PLRHPGMPKG LAHQLASPGL TTLPEASDVG SITLALRTLG SFEFEGHSLT |
| 601 | QFVRHCADHF LNSEHKEIRM EAARTCSRLL TPSIHLISGH AHVVSQTAVQ VVADVLSKLL |
| 661 | VVGITDPDPD IRYCVLASLD ERFDAHLAQA ENLQALFVAL NDQVFEIREL AICTVGRLSS |
| 721 | MNPAFVMPFL RKMLIQILTE LEHSGIGRIK EQSARMLGHL VSNAPRLIRP YMEPILKALI |
| 781 | LKLKDPDPDP NPGVINNVLA TIGELAQVSG LEMRKWVDEL FIIIMDMLQD SSLLAKRQVA |
| 841 | LWTLGQLVAS TGYVVEPYRK YPTLLEVLLN FLKTEQNQGT RREAIRVLGL LGALDPYKHK |
| 901 | VNIGMIDQSR DASAVSLSES KSSQDSSDYS TSEMLVNMGN LPLDEFYPAV SMVALMRIFR |
| 961 | DQSLSHHHTM VVQAITFIFK SLGLKCVQFL PQVMPTFLNV IRVCDGAIRE FLFQQLGMLV |
| 1021 | SFVKSHIRPY MDEIVTLMRE FWVMNTSIQS TIILLIEQIV VALGGEFKLY LPQLIPHMLR |
| 1081 | VFMHDNSPGR IVSIKLLAAI QLFGANLDDY LHLLLPPIVK LFDAPEAPLP SRKAALETVD |
| 1141 | RLTESLDFTD YASRIIHPIV RTLDQSPELR STAMDTLSSL VFQLGKKYQI FIPMVNKVLV |
| 1201 | RHRINHQRYD VLICRIVKGY TLADEEEDPL IYQHRMLRSG QGDALASGPV ETGPMKKLHV |
| 1261 | STINLQKAWG AARRVSKDDW LEWLRRLSLE LLKDSSSPSL RSCWALAQAY NPMARDLFNA |
| 1321 | AFVSCWSELN EDQQDELIRS IELALTSQDI AEVTQTLLNL AEFMEHSDKG PLPLRDDNGI |
| 1381 | VLLGERAAKC RAYAKALHYK ELEFQKGPTP AILESLISIN NKLQQPEAAA GVLEYAMKHF |
| 1441 | GELEIQATWY EKLHEWEDAL VAYDKKMDTN KDDPELMLGR MRCLEALGEW GQLHQQCCEK |
| 1501 | WTLVNDETQA KMARMAAAAA WGLGQWDSME EYTCMIPRDT HDGAFYRAVL ALHQDLFSLA |
| 1561 | QQCIDKARDL LDAELTAMAG ESYSRAYGAM VSCHMLSELE EVIQYKLVPE RREIIRQIWW |
| 1621 | ERLQGCQRIV EDWQKILMVR SLVVSPHEDM RTWLKYASLC GKSGRLALAH KTLVLLLGVD |
| 1681 | PSRQLDHPLP TVHPQVTYAY MKNMWKSARK IDAFQHMQHF VQTMQQQAQH AIATEDQQHK |
| 1741 | QELHKLMARC FLKLGEWQLN LQGINESTIP KVLQYYSAAT EHDRSWYKAW HAWAVMNFEA |
| 1801 | VLHYKHQNQA RDEKKKLRHA SGANITNATT AATTAATATT TASTEGSNSE SEAESTENSP |
| 1861 | TPSPLQKKVT EDLSKTLLMY TVPAVQGFFR SISLSRGNNL QDTLRVLTLW FDYGHWPDVN |
| 1921 | EALVEGVKAI QIDTWLQVIP QLIARIDTPR PLVGRLIHQL LTDIGRYHPQ ALIYPLTVAS |
| 1981 | KSTTTARHNA ANKILKNMCE HSNTLVQQAM MVSEELIRVA ILWHEMWHEG LEEASRLYFG |
| 2041 | ERNVKGMFEV LEPLHAMMER GPQTLKETSF NQAYGRDLME AQEWCRKYMK SGNVKDLTQA |
| 2101 | WDLYYHVFRR ISKQLPQLTS LELQYVSPKL LMCRDLELAV PGTYDPNQPI IRIQSIAPSL |
| 2161 | QVITSKQRPR KLTLMGSNGH EFVFLLKGHE DLRQDERVMQ LFGLVNTLLA NDPTSLRKNL |
| 2221 | SIQRYAVIPL STNSGLIGWV PHCDTLHALI RDYREKKKIL LNIEHRIMLR MAPDYDHLTL |
| 2281 | MQKVEVFEHA VNNTAGDDLA KLLWLKSPSS EVWFDRRTNY TRSLAVMSMV GYILGLGDRH |
| 2341 | PSNLMLDRLS GKILHIDFGD CFEVAMTREK FPEKIPFRLT RMLTNAMEVT GLDGNYRITC |
| 2401 | HTVMEVLREH KDSVMAVLEA FVYDPLLNWR LMDTNTKGNK RSRTRTDSYS AGQSVEILDG |
| 2461 | VELGEPAHKK TGTTVPESIH SFIGDGLVKP EALNKKAIQI INRVRDKLTG RDFSHDDTLD |
| 2521 | VPTQVELLIK QATSHENLCQ CYIGWCPFW |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding NF-kappa-B inhibitor alpha (Homo sapiens), such as one having NCBI Reference Sequence: NP_065390.1:

| | |
|---|---|
| 1 | MFQAAERPQE WAMEGPRDGL KKERLLDDRH DSGLDSMKDE EYEQMVKELQ EIRLEPQEVP |
| 61 | RGSEPWKQQL TEDGDSFLHL AIIHEEKALT MEVIRQVKGD LAFLNFQNNL QQTPLHLAVI |
| 121 | TNQPEIAEAL LGAGCDPELR DFRGNTPLHL ACEQGCLASV GVLTQSCTTP HLHSILKATN |
| 181 | YNGHTCLHLA SIHGYLGIVE LLVSLGADVN AQEPCNGRTA LHLAVDLQNP DLVSLLLKCG |
| 241 | ADVNRVTYQG YSPYQLTWGR PSTRIQQQLG QLTLENLQML PESEDEESYD TESEFTEFTE |
| 301 | DELPYDDCVF GGQRLTL |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding programmed cell death protein 1 precursor (Homo sapiens), such as one having NCBI Reference Sequence: NP_005009.2:

| | |
|---|---|
| 1 | MQIPQAPWPV VWAVLQLGWR PGWFLDSPDR PWNPPTFSPA LLVVTEGDNA TFTCSFSNTS |
| 61 | ESFVLNWYRM SPSNQTDKLA AFPEDRSQPG QDCRFRVTQL PNGRDFHMSV VRARRNDSGT |
| 121 | YLCGAISLAP KAQIKESLRA ELRVTERRAE VPTAHPSPSP RPAGQFQTLV VGVVGGLLGS |
| 181 | LVLLVWVLAV ICSRAARGTI GARRTGQPLK EDPSAVPVFS VDYGELDFQW REKTPEPPVP |
| 241 | CVPEQTEYAT IVFPSGMGTS SPARRGSADG PRSAQPLRPE DGHCSWPL |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as in a gene encoding 1-phosphatidylinositol 4,5-bisphosphate phosphodiesterase gamma-1 isoform a (Homo sapiens), such as one having NCBI Reference Sequence: NP_002651.2:

| | |
|---|---|
| 1 | MAGAASPCAN GCGPGAPSDA EVLHLCRSLE VGTVMTLFYS KKSQRPERKT FQVKLETRQI |
| 61 | TWSRGADKIE GAIDIREIKE IRPGKTSRDF DRYQEDPAFR PDQSHCFVIL YGMEFRLKTL |
| 121 | SLQATSEDEV NMWIKGLTWL MEDTLQAPTP LQIERWLRKQ FYSVDRNRED RISAKDLKNM |
| 181 | LSQVNYRVPN MRFLRERLTD LEQRSGDITY GQFAQLYRSL MYSAQKTMDL PFLEASTLRA |
| 241 | GERPELCRVS LPEFQQFLLD YQGELWAVDR LQVQEFMLSF LRDPLREIEE PYFFLDEFVT |
| 301 | FLFSKENSVW NSQLDAVCPD TMNNPLSHYW ISSSHNTYLT GDQFSSESSL EAYARCLRMG |
| 361 | CRCIELDCWD GPDGMPVIYH GHTLTTKIKF SDVLHTIKEH AFVASEYPVI LSIEDHCSIA |
| 421 | QQRNMAQYFK KVLGDTLLTK PVEISADGLP SPNQLKRKIL IKHKKLAEGS AYEEVPTSMM |
| 481 | YSENDISNSI KNGILYLEDP VNHEWYPHYF VLTSSKIYYS EETSSDQGNE DEEEPKEVSS |
| 541 | STELHSNEKW FHGKLGAGRD GRHIAERLLT EYCIETGAPD GSFLVRESET FVGDYTLSFW |
| 601 | RNGKVQHCRI HSRQDAGTPK FFLTDNLVFD SLYDLITHYQ QVPLRCNEFE MRLSEPVPQT |
| 661 | NAHESKEWYH ASLTRAQAEH MLMRVPRDGA FLVRKRNEPN SYAISFRAEG KIKHCRVQQE |
| 721 | GQTVMLGNSE FDSLVDLISY YEKHPLYRKM KLRYPINEEA LEKIGTAEPD YGALYEGRNP |
| 781 | GFYVEANPMP TFKCAVKALF DYKAQREDEL TFIKSAIIQN VEKQEGGWWR GDYGGKKQLW |
| 841 | FPSNYVEEMV NPVALEPERE HLDENSPLGD LLRGVLDVPA CQIAIRPEGK NNRLFVFSIS |
| 901 | MASVAHWSLD VAADSQEELQ DWVKKIREVA QTADARLTEG KIMERRKKIA LELSELVVYC |
| 961 | RPVPFDEEKI GTERACYRDM SSFPETKAEK YVNKAKGKKF LQYNRLQLSR IYPKGQRLDS |
| 1021 | SNYDPLPMWI CGSQLVALNF QTPDKPMQMN QALFMTGRHC GYVLQPSTMR DEAFDPFDKS |
| 1081 | SLRGLEPCAI SIEVLGARHL PKNGRGIVCP FVEIEVAGAE YDSTKQKTEF VVDNGLNPVW |
| 1141 | PAKPFHFQIS NPEFAFLRFV VYEEDMFSDQ NFLAQATFPV KGLKTGYRAV PLKNNYSEDL |
| 1201 | ELASLLIKID IFPAKQENGD LSPFSGTSLR ERGSDASGQL FHGRAREGSF ESRYQQPFED |
| 1261 | FRISQEHLAD HFDSRERRAP RRTRVNGDNR L |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding PR domain zinc finger protein 1 isoform 1 (Homo sapiens), such as one having NCBI Reference Sequence: NP_001189.2:

| | |
|---|---|
| 1 | MLDICLEKRV GTTLAAPKCN SSTVRFQGLA EGTKGTMKMD MEDADMTLWT EAEFEEKCTY |
| 61 | IVNDHPWDSG ADGGTSVQAE ASLPRNLLFK YATNSEEVIG VMSKEYIPKG TRFGPLIGEI |
| 121 | YTNDTVPKNA NRKYFWRIYS RGELHHFIDG FNEEKSNWMR YVNPAHSPRE QNLAACQNGM |
| 181 | NIYFYTIKPI PANQELLVWY CRDFAERLHY PYPGELTMMN LTQTQSSLKQ PSTEKNELCP |
| 241 | KNVPKREYSV KEILKLDSNP SKGKDLYRSN ISPLTSEKDL DDFRRRGSPE MPFYPRVVYP |
| 301 | IRAPLPEDFL KASLAYGIER PTYITRSPIP SSTTPSPSAR SSPDQSLKSS SPHSSPGNTV |
| 361 | SPVGPGSQEH RDSYAYLNAS YGTEGLGSYP GYAPLPHLPP AFIPSYNAHY PKFLLPPYGM |
| 421 | NCNGLSAVSS MNGINNFGLF PRLCPVYSNL LGGGSLPHPM LNPTSLPSSL PSDGARRLLQ |
| 481 | PEHPREVLVP APHSAFSFTG AAASMKDKAC SPTSGSPTAG TAATAEHVVQ PKATSAAMAA |
| 541 | PSSDEAMNLI KNKRNMTGYK TLPYPLKKQN GKIKYECNVC AKTFGQLSNL KVHLRVHSGE |
| 601 | RPFKCQTCNK GFTQLAHLQK HYLVHTGEKP HECQVCHKRF SSTSNLKTHL RLHSGEKPYQ |
| 661 | CKVCPAKFTQ FVHLKLHKRL HTRERPHKCS QCHKNYIHLC SLKVHLKGNC AAAPAPGLPL |
| 721 | EDLTRINEEI EKFDISDNAD RLEDVEDDIS VISVVEKEIL AVVRKEKEET GLKVSLQRNM |
| 781 | GNGLLSSGCS LYESSDLPLM KLPPSNPLPL VPVKVKQETV EPMDP |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding receptor-type tyrosine-protein phosphatase C isoform 1 precursor (Homo sapiens), such as one having NCBI Reference Sequence NP_002829.3:

| | |
|---|---|
| 1 | MTMYLWLKLL AFGFAFLDTE VFVTGQSPTP SPTGLTTAKM PSVPLSSDPL PTHTTAFSPA |
| 61 | STFERENDFS ETTTSLSPDN TSTQVSPDSL DNASAFNTTG VSSVQTPHLP THADSQTPSA |
| 121 | GTDTQTFSGS AANAKLNPTP GSNAISDVPG ERSTASTFPT DPVSPLTTTL SLAHHSSAAL |
| 181 | PARTSNTTIT ANTSDAYLNA SETTTLSPSG SAVISTTTIA TTPSKPTCDE KYANITVDYL |
| 241 | YNKETKLFTA KLNVNENVEC GNNTCTNNEV HNLTECKNAS VSISHNSCTA PDKTLILDVP |
| 301 | PGVEKFQLHD CTQVEKADTT ICLKWKNIET FTCDTQNITY RFQCGNMIFD NKEIKLENLE |
| 361 | PEHEYKCDSE ILYNNHKFTN ASKIIKTDFG SPGEPQIIFC RSEAAHQGVI TWNPPQRSFH |
| 421 | NFTLCYIKET EKDCLNLDKN LIKYDLQNLK PYTKYVLSLH AYIIAKVQRN GSAAMCHFTT |
| 481 | KSAPPSQVWN MTVSMTSDNS MHVKCRPPRD RNGPHERYHL EVEAGNTLVR NESHKNCDFR |
| 541 | VKDLQYSTDY TFKAYFHNGD YPGEPFILHH STSYNSKALI AFLAFLIIVT SIALLVVLYK |
| 601 | IYDLHKKRSC NLDEQQELVE RDDEKQLMNV EPIHADILLE TYKRKIADEG RLFLAEFQSI |
| 661 | PRVFSKFPIK EARKPFNQNK NRYVDILPYD YNRVELSEIN GDAGSNYINA SYIDGFKEPR |
| 721 | KYIAAQGPRD ETVDDFWRMI WEQKATVIVM VTRCEEGNRN KCAEYWPSME EGTRAFGDVV |
| 781 | VKINQHKRCP DYIIQKLNIV NKKEKATGRE VTHIQFTSWP DHGVPEDPHL LLKLRRRVNA |
| 841 | FSNFFSGPIV VHCSAGVGRT GTYIGIDAML EGLEAENKVD VYGYVVKLRR QRCLMVQVEA |
| 901 | QYILIHQALV EYNQFGETEV NLSELHPYLH NMKKRDPPSE PSPLEAEFQR LPSYRSWRTQ |
| 961 | HIGNQEENKS KNRNSNVIPY DYNRVPLKHE LEMSKESEHD SDESSDDDSD SEEPSKYINA |
| 1021 | SFIMSYWKPE VMIAAQGPLK ETIGDFWQMI FQRKVKVIVM LTELKHGDQE ICAQYWGEGK |
| 1081 | QTYGDIEVDL KDTDKSSTYT LRVFELRHSK RKDSRTVYQY QYTNWSVEQL PAEPKELISM |
| 1141 | IQVVKQKLPQ KNSSEGNKHH KSTPLLIHCR DGSQQTGIFC ALLNLLESAE TEEVVDIFQV |
| 1201 | VKALRKARPG MVSTFEQYQF LYDVIASTYP AQNGQVKKNN HQEDKIEFDN EVDKVKQDAN |
| 1261 | CVNPLGAPEK LPEAKEQAEG SEPTSGTEGP EHSVNGPASP ALNQGS |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding ras-related C3 botulinum toxin substrate 2 (Homo sapiens), such as one having NCBI Reference Sequence: NP_002863.1:

| | |
|---|---|
| 1 | MQAIKCVVVG DGAVGKTCLL ISYTTNAFPG EYIPTVFDNY SANVMVDSKP VNLGLWDTAG |
| 61 | QEDYDRLRPL SYPQTDVFLI CFSLVSPASY ENVRAKWFPE VRHHCPSTPI ILVGTKLDLR |
| 121 | DDKDTIEKLK EKKLAPITYP QGLALAKEID SVKYLECSAL TQRGLKTVFD EAIRAVLCPQ |
| 181 | PTRQQKRACS LL |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding signal transducer and activator of transcription 3 isoform 1 (Homo sapiens), such as one having NCBI Reference Sequence: NP_644805.1:

| | |
|---|---|
| 1 | MAQWNQLQQL DTRYLEQLHQ LYSDSFPMEL RQFLAPWIES QDWAYAASKE SHATLVFHNL |
| 61 | LGEIDQQYSR FLQESNVLYQ HNLRRIKQFL QSRYLEKPME IARIVARCLW EESRLLQTAA |
| 121 | TAAQQGGQAN HPTAAVVTEK QQMLEQHLQD VRKRVQDLEQ KMKVVENLQD DFDFNYKTLK |
| 181 | SQGDMQDLNG NNQSVTRQKM QQLEQMLTAL DQMRRSIVSE LAGLLSAMEY VQKTLTDEEL |
| 241 | ADWKRRQQIA CIGGPPNICL DRLENWITSL AESQLQTRQQ IKKLEELQQK VSYKGDPIVQ |
| 301 | HRPMLEERIV ELFRNLMKSA FVVERQPCMP MHPDRPLVIK TGVQFTTKVR LLVKFPELNY |
| 361 | QLKIKVCIDK DSGDVAALRG SRKFNILGTN TKVMNMEESN NGSLSAEFKH LTLREQRCGN |
| 421 | GGRANCDASL IVTEELHLIT FETEVYHQGL KIDLETHSLP VVVISNICQM PNAWASILWY |
| 481 | NMLTNNPKNV NFFTKPPIGT WDQVAEVLSW QFSSTTKRGL SIEQLTTLAE KLLGPGVNYS |
| 541 | GCQITWAKFC KENMAGKGFS FWVWLDNIID LVKKYILALW NEGYIMGFIS KERERAILST |
| 601 | KPPGTFLLRF SESSKEGGVT FTWVEKDISG KTQIQSVEPY TKQQLNNMSF AEIIMGYKIM |
| 661 | DATNILVSPL VYLYPDIPKE EAFGKYCRPE SQEHPEADPG SAAPYLKTKF ICVTPTTCSN |
| 721 | TIDLPMSPRT LDSLMQFGNN GEGAEPSAGG QFESLTFDME LTSECATSPM |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding proto-oncogene vav isoform 1 (Homo sapiens), such as one having NCBI Reference Sequence NP_005419.2:

| | |
|---|---|
| 1 | MELWRQCTHW LIQCRVLPPS HRVTWDGAQV CELAQALRDG VLLCQLLNNL LPHAINLREV |
| 61 | NLRPQMSQFL CLKNIRTFLS TCCEKFGLKR SELFEAFDLF DVQDFGKVIY TLSALSWTPI |
| 121 | AQNRGIMPFP TEEESVGDED IYSGLSDQID DTVEEDEDLY DCVENEEAEG DEIYEDLMRS |
| 181 | EPVSMPPKMT EYDKRCCCLR EIQQTEEKYT DTLGSIQQHF LKPLQRFLKP QDIEIIFINI |
| 241 | EDLLRVHTHF LKEMKEALGT PGAANLYQVF IKYKERFLVY GRYCSQVESA SKHLDRVAAA |
| 301 | REDVQMKLEE CSQRANNGRF TLRDLLMVPM QRVLKYHLLL QELVKHTQEA MEKENLRLAL |
| 361 | DAMRDLAQCV NEVKRDNETL RQITNFQLSI ENLDQSLAHY GRPKIDGELK ITSVERRSKM |
| 421 | DRYAFLLDKA LLICKRRGDS YDLKDFVNLH SFQVRDDSSG DRDNKKWSHM FLLIEDQGAQ |
| 481 | GYELFFKTRE LKKKWMEQFE MAISNIYPEN ATANGHDFQM FSFEETTSCK ACQMLLRGTF |
| 541 | YQGYRCHRCR ASAHKECLGR VPPCGRHGQD FPGTMKKDKL HRRAQDKKRN ELGLPKMEVF |
| 601 | QEYYGLPPPP GAIGPFLRLN PGDIVELTKA EAEQNWWEGR NTSTNEIGWF PCNRVKPYVH |
| 661 | GPPQDLSVHL WYAGPMERAG AESILANRSD GTFLVRQRVK DAAEFAISIK YNVEVKHIKI |
| 721 | MTAEGLYRIT EKKAFRGLTE LVEFYQQNSL KDCFKSLDTT LQFPFKEPEK RTISRPAVGS |
| 781 | TKYFGTAKAR YDFCARDRSE LSLKEGDIIK ILNKKGQQGW WRGEIYGRVG WFPANYVEED |
| 841 | YSEYC |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding tyrosine-protein kinase ZAP-70 isoform 1 (Homo sapiens), such as one having NCBI Reference Sequence: NP 001070.2:

| | |
|---|---|
| 1 | MPDPAAHLPF FYGSISRAEA EEHLKLAGMA DGLFLLRQCL RSLGGYVLSL VHDVRFHHFP |
| 61 | IERQLNGTYA IAGGKAHCGP AELCEFYSRD PDGLPCNLRK PCNRPSGLEP QPGVFDCLRD |
| 121 | AMVRDYVRQT WKLEGEALEQ AIISQAPQVE KLIATTAHER MPWYHSSLTR EEAERKLYSG |
| 181 | AQTDGKFLLR PRKEQGTYAL SLIYGKTVYH YLISQDKAGK YCIPEGTKFD TLWQLVEYLK |
| 241 | LKADGLIYCL KEACPNSSAS NASGAAAPTL PAHPSTLTHP QRRIDTLNSD GYTPEPARIT |
| 301 | SPDKPRPMPM DTSVYESPYS DPEELKDKKL FLKRDNLLIA DIELGCGNFG SVRQGVYRMR |
| 361 | KKQIDVAIKV LKQGTEKADT EEMMREAQIM HQLDNPYIVR LIGVCQAEAL MLVMEMAGGG |
| 421 | PLHKFLVGKR EEIPVSNVAE LLHQVSMGMK YLEEKNFVHR DLAARNVLLV NRHYAKISDF |
| 481 | GLSKALGADD SYYTARSAGK WPLKWYAPEC INFRKFSSRS DVWSYGVTMW EALSYGQKPY |
| 541 | KKMKGPEVMA FIEQGKRMEC PPECPPELYA LMSDCWIYKW EDRPDFLTVE QRMRACYYSL |
| 601 | ASKVEGPPGS TQKAEAACA |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding tumor necrosis factor receptor superfamily member 10A (Homo sapiens), such as one having NCBI Reference Sequence: NP_003835.3:

| | |
|---|---|
| 1 | MAPPPARVHL GAFLAVTPNP GSAASGTEAA AATPSKVWGS SAGRIEPRGG GRGALPTSMG |
| 61 | QHGPSARARA GRAPGPRPAR EASPRLRVHK TFKFVVVGVL LQVVPSSAAT IKLHDQSIGT |
| 121 | QQWEHSPLGE LCPPGSHRSE HPGACNRCTE GVGYTNASNN LFACLPCTAC KSDEEERSPC |
| 181 | TTTRNTACQC KPGTFRNDNS AEMCRKCSRG CPRGMVKVKD CTPWSDIECV HKESGNGHNI |
| 241 | WVILVVTLVV PLLLVAVLIV CCCIGSGCGG DPKCMDRVCF WRLGLLRGPG AEDNAHNEIL |
| 301 | SNADSLSTFV SEQQMESQEP ADLTGVTVQS PGEAQCLLGP AEAEGSQRRR LLVPANGADP |
| 361 | TETLMLFFDK FANIVPFDSW DQLMRQLDLT KNEIDVVRAG TAGPGDALYA MLMKWVNKTG |
| 421 | RNASIHTLLD ALERMEERHA REKIQDLLVD SGKFIYLEDG TGSAVSLE |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding signal transducer and activator of transcription 5B (Homo sapiens), such as one having NCBI Reference Sequence: NP_036580.2:

| | |
|---|---|
| 1 | MAVWIQAQQL QGEALHQMQA LYGQHFPIEV RHYLSQWIES QAWDSVDLDN PQENIKATQL |
| 61 | LEGLVQELQK KAEHQVGEDG FLLKIKLGHY ATQLQNTYDR CPMELVRCIR HILYNEQRLV |
| 121 | REANNGSSPA GSLADAMSQK HLQINQTFEE LRLVTQDTEN ELKKLQQTQE YFIIQYQESL |
| 181 | RIQAQFGPLA QLSPQERLSR ETALQQKQVS LEAWLQREAQ TLQQYRVELA EKHQKTLQLL |
| 241 | RKQQTIILDD ELIQWKRRQQ LAGNGGPPEG SLDVLQSWCE KLAEIIWQNR QQIRRAEHLC |
| 301 | QQLPIPGPVE EMLAEVNATI TDIISALVTS TFIIEKQPPQ VLKTQTKFAA TVRLLVGGKL |
| 361 | NVHMNPPQVK ATIISEQQAK SLLKNENTRN DYSGEILNNC CVMEYHQATG TLSAHFRNMS |
| 421 | LKRIKRSDRR GAESVTEEKF TILFESQFSV GGNELVFQVK TLSLPVVVIV HGSQDNNATA |
| 481 | TVLWDNAFAE PGRVPFAVPD KVLWPQLCEA LNMKFKAEVQ SNRGLTKENL VFLAQKLFNN |
| 541 | SSSHLEDYSG LSVSWSQFNR ENLPGRNYTF WQWFDGVMEV LKKHLKPHWN DGAILGFVNK |
| 601 | QQAHDLLINK PDGTFLLRFS DSEIGGITIA WKFDSQERMF WNLMPFTTRD FSIRSLADRL |
| 661 | GDLNYLIYVF PDRPKDEVYS KYYTPVPCES ATAKAVDGYV KPQIKQVVPE FVNASADAGG |
| 721 | GSATYMDQAP SPAVCPQAHY NMYPQNPDSV LDTDGDFDLE DTMDVARRVE ELLGRPMDSQ |
| 781 | WIPHAQS |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding cytokine receptor common subunit gamma precursor (Homo sapiens), for instance NCBI Reference Sequence. NP_00197.1:

| | |
|---|---|
| 1 | MLKPSLPFTS LLFLQLPLLG VGLNTTILTP NGNEDTTADF FLTTMPTDSL SVSTLPLPEV |
| 61 | QCFVFNVEYM NCTWNSSSEP QPTNLTLHYW YKNSDNDKVQ KCSHYLFSEE ITSGCQLQKK |
| 121 | EIHLYQTFVV QLQDPREPRR QATQMLKLQN LVIPWAPENL TLHKLSESQL ELNWNNRFLN |
| 181 | HCLEHLVQYR TDWDHSWTEQ SVDYRHKFSL PSVDGQKRYT FRVRSRFNPL CGSAQHWSEW |
| 241 | SHPIHWGSNT SKENPFLFAL EAVVISVGSM GLIISLLCVY FWLERTMPRI PTLKNLEDLV |
| 301 | TEYHGNFSAW SGVSKGLAES LQPDYSERLC LVSEIPPKGG ALGEGPGASP CNQHSPYWAP |
| 361 | PCYTLKPET |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding phosphatidylinositol 4,5-bisphosphate 3-kinase catalytic subunit gamma isoform (Homo sapiens), for example, NCBI Reference Sequence: NP_001269355.1:

| | |
|---|---|
| 1 | MELENYKQPV VLREDNCRRR RRMKPRSAAA SLSSMELIPI EFVLPTSQRK CKSPETALLH |
| 61 | VAGHGNVEQM KAQVWLRALE TSVAADFYHR LGPHHFLLLY QKKGQWYEIY DKYQVVQTLD |
| 121 | CLRYWKATHR SPGQIHLVQR HPPSEESQAF QRQLTALIGY DVTDVSNVHD DELEFTRRGL |
| 181 | VTPRMAEVAS RDPKLYAMHP WVTSKPLPEY LWKKIANNCI FIVIHRSTTS QTIKVSPDDT |
| 241 | PGAILQSFFT KMAKKKSLMD IPESQSEQDF VLRVCGRDEY LVGETPIKNF QWVRHCLKNG |
| 301 | EEIHVVLDTP PDPALDEVRK EEWPLVDDCT GVTGYHEQLT IHGKDHESVF TVSLWDCDRK |
| 361 | FRVKIRGIDI PVLPRNTDLT VFVEANIQHG QQVLCQRRTS PKPFTEEVLW NVWLEFSIKI |
| 421 | KDLPKGALLN LQIYCGKAPA LSSKASAESP SSESKGKVQL LYYVNLLLID HRFLLRRGEY |
| 481 | VLHMWQISGK GEDQGSFNAD KLTSATNPDK ENSMSISILL DNYCHPIALP KHQPTPDPEG |
| 541 | DRVRAEMPNQ LRKQLEAIIA TDPLNPLTAE DKELLWHFRY ESLKHPKAYP KLFSSVKWGQ |
| 601 | QEIVAKTYQL LARREVWDQS ALDVGLTMQL LDCNFSDENV RAIAVQKLES LEDDDVLHYL |
| 661 | LQLVQAVKFE PYHDSALARF LLKRGLRNKR IGHFLFWFLR SEIAQSRHYQ QRFAVILEAY |
| 721 | LRGCGTAMLH DFTQQVQVIE MLQKVTLDIK SLSAEKYDVS SQVISQLKQK LENLQNSQLP |
| 781 | ESFRVPYDPG LKAGALAIEK CKVMASKKKP LWLEFKCADP TALSNETIGI IFKHGDDLRQ |
| 841 | DMLILQILRI MESIWETESL DLCLLPYGCI STGDKIGMIE IVKDATTIAK IQQSTVGNTG |
| 901 | AFKDEVLNHW LKEKSPTEEK FQAAVERFVY SCAGYCVATF VLGIGDRHND NIMITETGNL |
| 961 | FHIDFGHILG NYKSFLGINK ERVPFVLTPD FLFVMGTSGK KTSPHFQKFQ DICVKAYLAL |
| 1021 | RHHTNLLIIL FSMMLMTGMP QLTSKEDIEY IRDALTVGKN EEDAKKYFLD QIEVCRDKGW |
| 1081 | TVQFNWFLHL VLGIKQGEKH SA |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding phosphatidylinositol 4,5-bisphosphate 3-kinase catalytic subunit delta isoform (Homo sapiens), for example, NCBI Reference Sequence: NP_005017:

| | |
|---|---|
| 1 | MPPGVDCPME FWTKEENQSV VVDFLLPTGV YLNFPVSRNA NLSTIKQLLW HRAQYEPLFH |
| 61 | MLSGPEAYVF TCINQTAEQQ ELEDEQRRLC DVQPFLPVLR LVAREGDRVK KLINSQISLL |
| 121 | IGKGLHEFDS LCDPEVNDFR AKMCQFCEEA AARRQQLGWE AWLQYSFPLQ LEPSAQTWGP |
| 181 | GTLRLPNRAL LVNVKFEGSE ESFTFQVSTK DVPLALMACA LRKKATVFRQ PLVEQPEDYT |
| 241 | LQVNGRHEYL YGSYPLCQFQ YICSCLHSGL TPHLTMVHSS SILAMRDEQS NPAPQVQKPR |
| 301 | AKPPPIPAKK PSSVSLWSLE QPFRIELIQG SKVNADERMK LVVQAGLFHG NEMLCKTVSS |
| 361 | SEVSVCSEPV WKQRLEFDIN ICDLPRMARL CFALYAVIEK AKKARSTKKK SKKADCPIAW |
| 421 | ANLMLFDYKD QLKTGERCLY MWPSVPDEKG ELLNPTGTVR SNPNTDSAAA LLICLPEVAP |
| 481 | HPVYYPALEK ILELGRHSEC VHVTEEEQLQ LREILERRGS GELYEHEKDL VWKLRHEVQE |
| 541 | HFPEALARLL LVTKWNKHED VAQMLYLLCS WPELPVLSAL ELLDFSFPDC HVGSFAIKSL |
| 601 | RKLTDDELFQ YLLQLVQVLK YESYLDCELT KFLLDRALAN RKIGHFLFWH LRSEMHVPSV |
| 661 | ALRFGLILEA YCRGSTHHMK VLMKQGEALS KLKALNDFVK LSSQKTPKPQ TKELMHLCMR |
| 721 | QEAYLEALSH LQSPLDPSTL LAEVCVEQCT FMDSKMKPLW IMYSNEEAGS GGSVGIIFKN |
| 781 | GDDLRQDMLT LQMIQLMDVL WKQEGLDLRM TPYGCLPTGD RTGLIEVVLR SDTIANIQLN |
| 841 | KSNMAATAAF NKDALLNWLK SKNPGEALDR AIEEFTLSCA GYCVATYVLG IGDRHSDNIM |
| 901 | IRESGQLFHI DFGHFLGNFK TKFGINRERV PFILTYDFVH VIQQGKTNNS EKFERFRGYC |
| 961 | ERAYTILRRH GLLFLHLFAL MRAAGLPELS CSKDIQYLKD SLALGKTEEE ALKHFRVKFN |
| 1021 | EALRESWKTK VNWLAHNVSK DNRQ |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding proto-oncogen vav (Homo sapiens), for example, NCBI Reference Sequence: NP_05419:

| | |
|---|---|
| 1 | MELWRQCTHW LIQCRVLPPS HRVTWDGAQV CELAQALRDG VLLCQLLNNL LPHAINLREV |
| 61 | NLRPQMSQFL CLKNIRTFLS TCCEKFGLKR SELFEAFDLF DVQDFGKVIY TLSALSWTPI |
| 121 | AQNRGIMPFP TEEESVGDED IYSGLSDQID DTVEEDEDLY DCVENEEAEG DEIYEDLMRS |
| 181 | EPVSMPPKMT EYDKRCCCLR EIQQTEEKYT DTLGSIQQHF LKPLQRFLKP QDIEIIFINI |
| 241 | EDLLRVHTHF LKEMKEALGT PGAANLYQVF IKYKERFLVY GRYCSQVESA SKHLDRVAAA |
| 301 | REDVQMKLEE CSQRANNGRF TLRDLLMVPM QRVLKYHLLL QELVKHTQEA MEKENLRLAL |
| 361 | DAMRDLAQCV NEVKRDNETL RQITNFQLSI ENLDQSLAHY GRPKIDGELK ITSVERRSKM |
| 421 | DRYAFLLDKA LLICKRRGDS YDLKDFVNLH SFQVRDDSSG DRDNKKWSHM FLLIEDQGAQ |
| 481 | GYELFFKTRE LKKKWMEQFE MAISNIYPEN ATANGHDFQM FSFEETTSCK ACQMLLRGTF |
| 541 | YQGYRCHRCR ASAHKECLGR VPPCGRHGQD FPGTMKKDKL HRRAQDKKRN ELGLPKMEVF |
| 601 | QEYYGLPPPP GAIGPFLRLN PGDIVELTKA EAEQNWWEGR NTSTNEIGWF PCNRVKPYVH |
| 661 | GPPQDLSVHL WYAGPMERAG AESILANRSD GTFLVRQRVK DAAEFAISIK YNVEVKHIKI |
| 721 | MTAEGLYRIT EKKAFRGLTE LVEFYQQNSL KDCFKSLDTT LQFPFKEPEK RTISRPAVGS |
| 781 | TKYFGTAKAR YDFCARDRSE LSLKEGDIIK ILNKKGQQGW WRGEIYGRVG WFPANYVEED |
| 841 | YSEYC |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding Lymphocyte cytosolic protein 2 (Homo sapiens), for example, NCBI Reference Sequence. NP_005556:

| | |
|---|---|
| 1 | MALRNVPFRS EVLGWDPDSL ADYFKKLNYK DCEKAVKKYH IDGARFLNLT ENDIQKFPKL |
| 61 | RVPILSKLSQ EINKNEERRS IFTRKPQVPR FPEETESHEE DNGGWSSFEE DDYESPNDDQ |
| 121 | DGEDDGDYES PNEEEEAPVE DDADYEPPPS NDEEALQNSI LPAKPFPNSN SMYIDRPPSG |
| 181 | KTPQQPPVPP QRPMAALPPP PAGRNHSPLP PPQTNHEEPS RSRNHKTAKL PAPSIDRSTK |
| 241 | PPLDRSLAPF DREPFTLGKK PPFSDKPSIP AGRSLGEHLP KIQKPPLPPT TERHERSSPL |
| 301 | PGKKPPVPKH GWGPDRREND EDDVHQRPLP QPALLPMSSN TFPSRSTKPS PMNPLPSSHM |
| 361 | PGAFSESNSS FPQSASLPPY FSQGPSNRPP IRAEGRNFPL PLPNKPRPPS PAEEENSLNE |
| 421 | EWYVSYITRP EAEAALRKIN QDGTFLVRDS SKKTTTNPYV LMVLYKDKVY NIQIRYQKES |
| 481 | QVYLLGTGLR GKEDFLSVSD IIDYFRKMPL LLIDGKNRGS RYQCTLTHAA GYP |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a modification such as one in a gene encoding Phospholipase C, gamma 1, also known as PLCG1 (Homo sapiens), for example, NCBI Reference Sequence: NP_002651:

| | |
|---|---|
| 1 | MAGAASPCAN GCGPGAPSDA EVLHLCRSLE VGTVMTLFYS KKSQRPERKT FQVKLETRQI |
| 61 | TWSRGADKIE GAIDIREIKE IRPGKTSRDF DRYQEDPAFR PDQSHCFVIL YGMEFRLKTL |
| 121 | SLQATSEDEV NMWIKGLTWL MEDTLQAPTP LQIERWLRKQ FYSVDRNRED RISAKDLKNM |
| 181 | LSQVNYRVPN MRFLRERLTD LEQRSGDITY GQFAQLYRSL MYSAQKTMDL PFLEASTLRA |
| 241 | GERPELCRVS LPEFQQFLLD YQGELWAVDR LQVQEFMLSF LRDPLREIEE PYFFLDEFVT |
| 301 | FLFSKENSVW NSQLDAVCPD TMNNPLSHYW ISSSHNTYLT GDQFSSESSL EAYARCLRMG |
| 361 | CRCIELDCWD GPDGMPVIYH GHTLTTKIKF SDVLHTIKEH AFVASEYPVI LSIEDHCSIA |
| 421 | QQRNMAQYFK KVLGDTLLTK PVEISADGLP SPNQLKRKIL IKHKKLAEGS AYEEVPTSMM |
| 481 | YSENDISNSI KNGILYLEDP VNHEWYPHYF VLTSSKIYYS EETSSDQGNE DEEEPKEVSS |
| 541 | STELHSNEKW FHGKLGAGRD GRHIAERLLT EYCIETGAPD GSFLVRESET FVGDYTLSFW |
| 601 | RNGKVQHCRI HSRQDAGTPK FFLTDNLVFD SLYDLITHYQ QVPLRCNEFE MRLSEPVPQT |
| 661 | NAHESKEWYH ASLTRAQAEH MLMRVPRDGA FLVRKRNEPN SYAISFRAEG KIKHCRVQQE |
| 721 | GQTVMLGNSE FDSLVDLISY YEKHPLYRKM KLRYPINEEA LEKIGTAEPD YGALYEGRNP |
| 781 | GFYVEANPMP TFKCAVKALF DYKAQREDEL TFIKSAIIQN VEKQEGGWWR GDYGGKKQLW |
| 841 | FPSNYVEEMV NPVALEPERE HLDENSPLGD LLRGVLDVPA CQIAIRPEGK NNRLFVFSIS |
| 901 | MASVAHWSLD VAADSQEELQ DWVKKIREVA QTADARLTEG KIMERRKKIA LELSELVVYC |
| 961 | RPVPFDEEKI GTERACYRDM SSFPETKAEK YVNKAKGKKF LQYNRLQLSR IYPKGQRLDS |
| 1021 | SNYDPLPMWI CGSQLVALNF QTPDKPMQMN QALFMTGRHC GYVLQPSTMR DEAFDPFDKS |
| 1081 | SLRGLEPCAI SIEVLGARHL PKNGRGIVCP FVEIEVAGAE YDSTKQKTEF VVDNGLNPVW |
| 1141 | PAKPFHFQIS NPEFAFLRFV VYEEDMFSDQ NFLAQATFPV KGLKTGYRAV PLKNNYSEDL |
| 1201 | ELASLLIKID IFPAKQENGD LSPFSGTSLR ERGSDASGQL FHGRAREGSF ESRYQQPFED |
| 1261 | FRISQEHLAD HFDSRERRAP RRTRVNGDNR L |

(SEQ ID NO:), a different isoform of the protein, or a polypeptide having at least 80%, 82%, 85%, 86%, 88%, 90%, 92%, 94%, 95%, 97%, 98% or 99% amino acid sequence identity thereto.

### Formulations and Dosages

The modified immune cells can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

In one embodiment, the immune cells may be administered by infusion or injection. Solutions of the immune cells can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze-drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the cells may be from 1×10⁴ to 1×10⁶, 1×10⁵ to 1×10⁷, 1×10⁶ to 1×10⁸, 1×10⁷ to 1×10⁹, 1×10⁸ to 1×10¹⁰, 1×10¹⁰ to 1×10¹², or 1×10¹¹ to 1×10¹⁵ cells.

The amount of for use alone or with other agents will vary with the route of administration, the nature of the condition being treated and the age and condition of the patient and will be ultimately at the discretion of the attendant physician or clinician.

The invention will be described by the following non-limiting example.

### Example

### Methods

### Lentiviral Plasmids and Cloning for ABE and CBE Base Editors.

The lentiviral transfer plasmids containing base editors were generated using pZR071 (Addgene 180264) as a backbone. The pZR071 plasmid was digested with EcoRI and KpnI, followed by ligation of a cassette composed of the EF1a promoter, base editor coding sequence, and a P2A-Blasticidin resistance element. For the plenti-CR033 transfer plasmid, the ABE8e(V106W) coding sequence was obtained from ABE8e(TadA-8e-V106W) (Addgene 138495). For the plenti-CR029 transfer plasmid, the evoCDA1-BE4max sequence was derived from pBT277_evoCDA1-BE4max-in-mammalian-cells (Addgene 122608). For the plenti-CR102 transfer plasmid, the nCas9 from plenti-CR033 was replaced with SpG, which was sourced from pCAG-CBE-4max-SpG-P2A-EGFP (RTW4552) (Addgene 139998).

### Library Cloning

A combination of literature review, gene ontology annotations and hits from previous CRISPR screens in T cells led to a list of 385 genes implicated in regulation of T cell activation, differentiation and function. For each gene we choose the longest annotated isoform and selected all NGG PAM guides which: (1) overlapped with the coding sequence plus 2&bp on the side of each exon to include splice sites; and (2) contained an editable base (A or C) in the expected editing window (position 3-8 for ABE and 1-13 for CBE). The addition of 1,000 non-targeting sgRNAs derived from the Brunello library resulted in a list of 117,249 unique sgRNAs. We added type II restriction sites and sequences for amplification, as previously described. The oligonucleotide pool was obtained from Agilent and amplified with KAPA HiFi HotStart (Roche 07958935001). The PCR product was purified and ligated into LentiGuide-Puro plasmid (Addgene 52963) backbone with the New England Biolabs NEB Golden Gate Assembly Kit (BsmBI-HF v2) (New England Biolabs, E1602L) per the manufacturer's instructions. Ligation product was transfected into Lucigen Endura electrocompetent cells (LGC, 60242-2) per the manufacturer's instructions. Cells were expanded at 30&C for 18&h and plasmid was extracted using the QIAGEN Plasmid Plus Mega Kit (Qiagen, 12981). Total amount of transfected cells was calculated by counting colonies of separate ampicillin plates at 1/10,000,000 and 1/100,000,000 dilution. We proceeded with next generation sequencing of the plasmid pool at coverage of >1,000×, confirming representation and distribution of sgRNAs. The plasmid pool was then used for lentivirus production as described under ‘Lentivirus production’. For screens using SpG nCas9, 57 gene hits from the NGG screen and CBLB were selected based on the effect of knockout guides. Guides were selected for the coding regions of these genes in an identical manner to the NGG library, only for NG PAM guides, an oligonucleotide pool containing 45,941 oligonucleotides was ordered similarly to the first screen using NGG nCas9. Library preparation and quality control was performed as described for the NGG library; read counts for quality control sequencing of the libraries were used.

### RNA Production

For experiments with base editor mRNA, an in!vitro transcription (IVT) plasmid containing ABE8e(V106W) with a mutated T7 promoter was designed and cloned as previously described. The IVT templates were produced by PCR of ABE8e(V106W) with the forward primer correcting the T7 mutation and reverse primer appending the polyA tail, thus the final PCR product contained WT T7 promoter, 5′ untranslated region including Kozak sequence, the codon-optimized ABE8e(V106W) coding sequence, 3′ untranslated region, and a 145-bp polyA tail. The PCR product was purified and stored at −20° C. until further use. IVT reactions were performed with the HiScribe T7 High Yield RNA Synthesis Kit (New England Biolabs, E2040S) under full substitution of UTP and in presence of 4&mM CleanCap (TriLink Biotechnologies, N-7113-5), as described. Transcribed mRNA was purified with lithium chloride and eluted in RNA storage solution (Fisher Scientific, AM7000). After quantification on a Nanodrop spectrophotometer and normalization to 1 μgμl⁻¹, mRNA product was assessed on an Agilent 4200 Tapestation system and subsequently stored at −80° C.

Lentivirus Production Lentiviral particles were produced as previously described. In brief, for a T75 flask, 12×10⁶ Lenti-X HEK 293T cells were seeded in 15&ml complete Opti-MEM. The next morning, cells were transfected with 12 μg transfer plasmid, 12 μg psPAX2 (Addgene 12260) and 5 μg pMD2.G (Addgene 12259) with 56 μl p3000 and 65 μl Lipofectamine 3000 (Fisher Scientific, L3000075). Six hours after transfection, medium was replaced with fresh cOpti-MEM supplemented with 1× viral boost (Alstem, VB100). Another 18 h later, virus containing supernatant was collected and spun down at 500 g for 5 min at 4° C. to pellet cell debris. Lentivirus-containing medium was moved to a new vessel and subsequently concentrated 100-fold using Lenti-X Concentrator (Takara Bio, 631232) per the manufacturer's instructions. Viral particles were stored at −80° C. until further use.

### T Cell Isolation and Culture

Human peripheral blood Leukopaks enriched for PBMCs were sourced from Stemcell Technologies (200-0092). The donors were chosen without regard to sex, gender, ethnicity or race. Donors for the screens were <60 years old and non-smokers. T!cells were isolated with the EasySep Human T cell isolation kit (100-069) or EasySep Human CD4+ T Cell Isolation Kit (Stemcell Technologies, 100-0696) per the manufacturer's instructions. Immediately after isolation, T cells were either frozen or used directly for in vitro experiments. Fresh T cells were seeded at 1×10⁶ cells per ml and subsequently used for experiments. When frozen, T cells were counted, spun down, and resuspended in Bambanker freezing medium at a concentration of 50×10⁶ cells per ml. T cells were stored at −80° C. for up to 2 months or in liquid nitrogen for longer periods. When needed, T cells were thawed and seeded at 1×10⁶ cells per ml, similar to fresh T cells. Unless otherwise indicated, T cells were cultured in complete X-VIVO 15 (cX-VIVO) consisting of X-VIVO 15 (Lonza Bioscience, 04-418Q) supplemented with 5% FCS (R&D systems, lot M19187), 4 mM N-acetyl-cysteine (VWR, VWRV0108-25G) and 55 μM 2-mercaptoethanol (Fisher Scientific, 21985023).

### Cell Lines

Lenti-X HEK 293T cells (Takara Bio, 632180) were cultured in complete DMEM consisting of high glucose DMEM (Fisher Scientific, 10566024) supplemented with 10% FCS (R&D systems, LOT M19187), 1× MEM Non-Essential Amino Acids Solution (Fisher Scientific, 11140050), 1 mM sodium pyruvate (Fisher Scientific, 11360070), 10&mM HEPES (Sigma Aldrich, H0887-100ML), 1,000&U&ml-1 penicillin-streptomycin (Fisher Scientific, 15140122). Lenti-X HEK 293T cells were subcultured every 2-3 days and maintained at a confluence of <70% for a maximum of 15 passages. A375-nRFP (a gift from A. Ashworth) cells were cultured in complete RPMI (cRPMI) consisting of RPMI (Fisher Scientific, 21870092) with 10% FCS, 55 μM 2-mercaptoethanol, 2&mM+-Glutamine (Fisher Scientific, 25030081), 1,000Uml⁻¹ penicillin-streptomycin, 1× MEM non-essential amino acids solution, 1 mM sodium pyruvate and 10 mM HEPES (Sigma Aldrich, H0887-100ML) and subcultured every 2-3 days to keep them at a confluency of <70%. Cell lines were tested negative for mycoplasma contamination.

Arrayed Base Editing with Lentivirus

Frozen human Pan CD3+ T!cells were thawed and activated with anti-CD3/anti-CD28 Dynabeads (Life Technologies, 40203D) in presence of 200IUml-1 IL-2. The next morning, plenti-CR029 or plenti-CR033 lentivirus was added to the cells at 2% v/v. The following day cells were subcultured 1/1 and IL-2 was replenished. Additionally, lentiguide-Puro with CD3-, CD5- and CD7-targeting sgRNAs were added. One day later, cells were split again 1:1 with half of the cells receiving blasticidin treatment at 10 μgml⁻¹ and all cells receiving puromycin treatment at 2 μgml⁻¹. Cells were subcultured and IL-2 was refreshed every two days and assessed by flow cytometry and Sanger sequencing seven days after initial T cell activation.

For testing our ABE SpG lentiviral base editing approach, we followed a similar strategy with viral particles produced from plenti-CR102.

### NGG Screen

CD4+ T cells from 3 human donors were isolated as described under ‘T cell isolation and culture’. For ABE screens, 150&×&10⁶ cells per donor were used for subsequent activation in cX-VIVO 15 at a concentration of 1×10⁶ cells per ml. For CBE screens, 225×10⁶ cells per donor were seeded under similar culture conditions. CD4+ T cell purity was confirmed by flow cytometry (>95%). Cells were subsequently activated with anti-CD3/anti-CD28 Dynabeads (Life Technologies, 40203D) at a 1:1 bead-to-cell ratio and with 200IUml⁻¹ IL-2 (R&D systems, 202-GMP-01M). Next morning, 2% v/v plenti-CR033 or 2.2% plenti-CR029 lentivirus was added for ABE and CBE screens, respectively. Two days later, 50% of the original culture volume cX-VIVO was added and IL-2 was supplemented for a final concentration of 200IUml⁻¹. 1CR005 library lentivirus was added at 1.3% v/v (corresponding to a multiplicity of infection (MOI)~0.3) and cells were mixed. Next day, cells were counted and fresh medium was added to keep the culture at 1×10⁶ cells per ml. IL-2 was supplemented to a final concentration of 200IUml⁻¹, puromycin and blasticidin were added to 2.5 μgml⁻¹ and 20 μgml⁻¹ final concentrations, respectively. Cells were subcultured and expanded every 2 days and kept at ~1×10⁶ per ml and 200IUml⁻¹ IL-2. Five days after addition of blasticidin and puromycin, cells from each screen were collected, pooled, and counted. Fresh medium without supplements was added to bring T cells to 2×10⁶ cells per ml. Next morning, T cells were restimulated with 6.25 μlml⁻¹ anti-CD3/anti-CD28/anti-CD2 Immunocult (Stemcell Technologies, 10990). For cytokine screens, Protein Transport Inhibitor (Becton Dickinson, 555029) was added at a 1/1,000 dilution 1&h after stimulation, followed by 9&h incubation. Cytokine screen cells were collected 10 h after stimulation and fixed and stained for FACS using the Cytofix/Cytoperm Fixation/Permeabilization Solution Kit (Becton Dickinson, 554714). For PD-1 and CD25 screens, cells were collected one day after activation, stained for Live/Dead and surface protein expression facs sorting and fixed in 4% paraformaldehyde for 30&min at 4&C. T cells were subsequently sorted into TNF, IFN-%, CD25 or PD-1 high and low bins. Over the whole course of the screen from initial activation and expansion to sorting, we maintained a coverage of >1,000× (cells/sgRNA) to ensure consistent representation of the library. After staining and fixation, T cells for all screens were washed twice in EasySep (1×PBS with 2% FCS and 1&mM EDTA) and sorted into the respective bin. Sorted T cells were spun down, and cell pellets were stored at −80° C. until further use.

### NG Screen

Fifty million human Pan CD3+ T cells from each two human donors were isolated as described under ‘T cell isolation and culture’. Subsequently, T cells were stimulated with anti-CD3/anti-CD28 Dynabeads at a 1:1 bead-to-cell ratio and 200&IU&ml-1 IL-2. Next day, plenti-CR102 ABE8e(V106W)-SpG base editor lentivirus was added at 2% v/v. One day later, T cells were infected with 1.5% v/v (corresponding to an MOI of ~0.6) of the NG base editor library lentivirus followed by selection with blasticidin and puromycin as described before. T cells were subcultured and expanded with fresh medium and IL-2 every 2-3 days and further processed as described under ‘NGG screen’. Over the whole course of the screen from initial T cell activation to sorting, a coverage of >1,000× (cells!per!sgRNA) was maintained to ensure representation of the library. Finally, human Pan CD3+ T cells were sub-sorted into CD4+ TNF-low and TNF-high as well as CD8+ TNF-low and TNF-high T cells. Extraction of genomic DNA and quantification of sgRNAs in each population was performed as described under ‘Genomic DNA extraction, library preparation and NGS for base editing screens’.

### Genomic DNA Extraction, Library Preparation and NGS for Base Editing Screens

Genomic DNA of sorted T!cell pellets was performed as described. For 5×10⁶ T cells, pellets were thawed and incubated overnight at 65° C. in 400 μl lysis buffer (Chip composition) and 16 μl of 5M NaCl. Next day, 32 μl RNase A (10mgml⁻¹ stock concentration) was added and incubated at 37° C. for 2 h followed by a 2 h 55° C. incubation after adding 16 μl (20mgml⁻¹) proteinase K. Genomic DNA was then separated with phenol:chloroform:isoamyl alcohol and precipitated with sodium acetate and washed with 70% ethanol. DNA was eluted in water and quantified on a Nanodrop Spectrophotometer. PCR for NGS was performed with KAPA HiFi HotStart polymerase, using previously established primers and PCR protocol with annealing temperature at 63° C. PCR products were purified by solid-phase reversible immobilization and further gel purified, diluted to 10&nM and submitted for NGS on a NovaSeq Instrument (Illumina) at the UCSF Center for Advanced Technologies. Targeted sequencing depth was 1,000× of sgRNA counts per sample.

Real-Time qPCR

T cells from four independent human donors were treated as described under ‘Arrayed base editing with mRNA’. One day after restimulation, RNA for expression analyses with qPCR was isolated with the Quick-RNA MicroPrep and RNA Clean and Concentrator-5 Kits (Zymo Research, R1051 and R1016) using TURBO DNase and 10× TURBO DNase Buffer (Invitrogen, AM2239). Real-time qPCR was performed using the Prime-Time One-Step RT-qPCR Master Mix (10007065) with pre-designed primer-probe pairs for PIK3CD, IL2RA and B2M (housekeeping) following the manufacturer's instructions. Expression relative to B2M was calculated with the 2-ΔΔC_{T} method.

Arrayed Base Editing with mRNA

For arrayed base editing experiments with ABE mRNA, fresh or previously frozen human Pan T cells were activated with a 1:1 bead-to-cell ratio with anti-CD3/CD28 Dynabeads (Thermo Fisher, 40203D) in the presence of 500IUml⁻¹ IL-2 at 1×10⁶ cells per ml. Two days after stimulation, T cells were magnetically de-beaded and taken up in P3 buffer with supplement (Lonza Bioscience, V4SP-3096) at 37.5×10⁶ cells per ml. Two micrograms of ABE mRNA mixed with 1.5 μg synthetic modified sgRNA (Synthego) was added per 20 μl cells, not exceeding 25 μl total per reaction. Cells were subsequently electroporated on a Lonza 4D Nucleofector using the electroporation code DS137. Immediately after electroporation, 100 μl warm complete X-VIVO15 was added to each electroporation well and cells were incubated for 15 min in a CO₂ incubator at 37° C. followed by distribution of each electroporation reaction into three wells of a 96-well flat bottom plate. Each 96 flat bottom well was brought to 200 μl cXVIVO15 and 100IUml⁻¹ IL-2. Cells were subcultured and expanded by addition of fresh medium and IL-2 every 2-3 days. Ten days after electroporation, cells were counted and normalized to 2×10⁶ live cells per ml through complete medium change and without the addition of IL-2. The next morning, cells were re-stimulated with indicated doses of anti-CD3/anti-CD28/anti-CD2 Immunocult (Stemcell Technologies, 10990). Cell pellets saved for genomic DNA analysis were spun down at 300 g for 5&min and resuspended in QuickExtract (VWR, 76081-766) following the manufacturer's recommended protocol. Next, cells were treated as described under ‘NGG screen’ for cytokine and surface marker flow cytometry and gated for CD4+ and CD8+ cells. sgRNA sequences used in arrayed experiments are provided.

### Cytotoxicity Assays

For in-vitro cytotoxicity assays, fresh or frozen T cells were stimulated as described under ‘NGG screen’. One day after stimulation, cells were infected with 1% v/v 100× concentrated lentivirus carrying an open reading frame element encoding the 1G4 T cell receptor that can recognize the NYESO1 cancer antigen in an HLA-A0201 context. One day later, cells were electroporated and cultured as described under ‘Arrayed base editing with mRNA’. Ten days after electroporation, 300 RFP-expressing A375 melanoma cells were seeded in 50& μl cRPMI per well in a 384 well plate. The outer two positions of the 384 well plate were filled with water and not used for analyses. The next morning, T cells were counted and 1G4 T!cell receptor expression was assessed by flow cytometry. TCR positive cells were used for normalization and added to A375 cells according to the indicated E:T ratios. The final volume for one well of a 384-well plate was 901. After adding the T cells on top of A375 cells, the plate was moved to an Incucyte live-cell imaging system (Sartorius) with assessment cycles every 6 h. A375 cells were automatically counted by the Incucyte instrument based on RFP expression and cell counts were exported for plotting.

### HDR-Mediated Knockins

Fresh pan T cells were isolated and activated as described above. Two days after activation, cells were electroporated in presence of Cas9 RNPs and HDR templates consisting of 100-bp single stranded oligonucleotides synthesized by IDT, as described58. Next, cells were expanded and restimulated for cytokine staining as described above and subsequently assessed for cytokine production and activation marker expression, similar to the description in ‘Arrayed base editing with mRNA’. Genomic DNA was isolated from cells and prepared for NGS as described above.

### Sorting and Flow Cytometry

FACS was performed on BD FACSAria Fusion cell sorters equipped with 70 μm nozzles at the Gladstone Flow Core. For each sorting bin, a cell-to-guide RNA ratio of >500 was targeted. Flow cytometry analyses were done using an Attune Nxt machine with plate reader function.

### Secreted Cytokine Analyses

For cytokine analyses in culture medium, cell culture plates were spun down at 500 g for 5 min and half of the supernatant was removed and stored at −80° C. All samples were analysed by Eve Technologies using Luminex xMAP technology. First, a titration series was run to determine optimal dilution factors and to avoid assay saturation. IL-6, IL-8 and IL17A were run at a twofold dilution. All other cytokines were analyzed at a 200-fold dilution.

### Base-Editing Screen Analyses

After sequencing, fastq files were analyzed with MAGeCK59. Samples with poor genomic DNA recovery after sorting (<100× coverage) were excluded from further analysis. First, the command MAGeCK count was used to assess fastq files, representing each sorting bin, for the presence of guideRNA sequences and count them. Next, MAGeCK test was run to assess the distribution of guideRNAs and comparisons across different sorting bins relative to distribution of non-targeting controls. The low bins were used as MAGeCK control input and high bins as treatment input for the NGG screen. The low bins were used as MAGeCK treatment input and high bins as control input for the NG screen. For plotting of the NG screen, log 2FC values were inverted. Therefore, a negative log 2FC or negative hit indicates enrichment in the low bins while positive hits are enriched in the high bins. For downstream analysis the bottom 2.5 percentile of guides were discarded. As coding regions were selected inclusively based on longest isoforms, for ease of interpretation and analysis, predicted amino-acid-level edit locations were mapped by pairwise alignment to the canonical sequence in UniProt for each protein and the canonical sequence positions used in all further analysis. For correlation of editing effects with BLOSUM62 substitution penalty, the highest penalty mutation was used for guides with multiple predicted mutations and the relative frequencies in calculated based on the single highest penalty mutation as well.

sgRNA Off-Target Analyses

To assess off target potential, each guide was searched against the human genome for off target sites with up to five mismatches using Cas-OFFinder. Afterwards, scoring of target sites was performed with the CFD algorithm. Guides were filtered out in off target analysis if there existed >5 off-target sites with a CFD score of 1.012.

### Evaluation of Base-Level Effects

To estimate the base-resolution effects in the screen analyses, we first listed all editable bases in the expected editing windows for each guide RNA (A in position 3-8 for ABE, or 3-9 for expanded window analysis and C in position 1-13 for CBE). Next, the effect of each guide RNA in every sample was quantified by dividing the normalized count in the high bin by the normalized count in the low bin. Normalized counts were obtained from the output file of the MAGeCK analysis, as described above. Following this, we log-normalized the effects of all guide RNAs pertaining to each gene and performed a regression analysis based on the presence of editable bases, using a multiple linear regression model. This allowed us to estimate base-level effect sizes as well as statistical significance (two-sided Wald test) for each base in each screen. (Supplementary Tables!6-8) In instances where two bases were covered by the same combination of guide RNAs and thus could not be distinguished, they were assigned identical effect sizes. For residue-level statistics, we averaged log 2FC (obtained from MAGeCK) from all non-terminating guides spanning a residue that induces a non-synonymous change, and combined the P!values (also from MAGeCK) using Fisher's method.

To test whether disease-causing human genetic variants were enriched among functional bases in our screening results, we compared our results with the variants in ClinVar. We downloaded the VCF file for variant functions from ClinVar on May 8, 2023. Our focus was on variants categorized as ‘pathogenic’, ‘likely-pathogenic’ or ‘benign’ in ClinVar. We restricted our comparison to non-synonymous variants that overlapped with the editing windows of our guide RNAs and had reference/alternative alleles matching our editing (A to G or T to C for ABE, and C to T or G to A for CBE). The overlap between ClinVar variants and variants in our screen filtered to a defined significance threshold was compared with the overlap between ClinVar variants and the remaining variants in the screening using Fisher's two-sided exact tests.

### Statistical and Reproducibility

Screen analysis was done with MAGeCK59 as described in the respective screen section. All other statistical analyses (correlation and significance testing) were done with the scipy.stats package.

### Nucleotide-Resolution Decoding of Human Immune Cell Gene Programs

Human T cells are critical effectors of immune protection from infections, autoimmune pathology, and cancer. In order to understand the genetic sequences that control immune cells in health and disease and discover sequences that can be re-written to enhance the next generation of adoptive cell therapies for complex human diseases ranging from cancer to autoimmune disorders, a broad range of CRISPR technologies—including CRISPR knockout (Shifrut et al., Cell, 2018) and knock-in (Roth et al., Cell, 2020), and CRISPR activation and CRISPR interference (Schmidt et al., Science, 2022) have been employed to identify key genes and pathways that govern T cell function. As described herein base editing technology was used to achieve nucleotide-resolution understanding of immune cell genetics and to fine-tune engineered cellular therapies.

### Create Nucleotide-Resolution Genetic Maps to Explore T Cell Functions and Inform Cell Engineering.

A base editor screening platform was developed in primary human T cells for comprehensive analysis of gene variants and programs that control T cell functions. As nucleotide-resolution maps in human T cells were generated, specific genetic sites were found that could be targeted in engineered cell therapies to confer and tune desired phenotypes.

### Dissect the Genetic Architecture of Autoimmune Disorders.

Despite widespread genome/exome sequencing of patients, genetic causes of complex autoimmune diseases remain elusive largely due to a lack of functional testing for each mutation, especially in non-coding genome elements. The base editing technology in human T cells can be used to test large numbers of variants introduced into endogenous disease loci for systematic assignment of variant causality. Dense sampling in disease-relevant regions allows for refined understanding of genome-wide association studies (GWAS) of complex diseases with the opportunity to nominate variants that enhance T cell functions for next-generation adoptive cellular therapies.

### Design and Manufacture Multiplexed Products to Fine-Tune T Cells for Adoptive Cell Therapies.

Today, clinical base editing efforts focus largely on correcting monogenic mutations or knocking out genes. Specific base edits can be designed to enhance immune cell functions to treat complex diseases. For example, cell therapies for viral targets and autoimmune diseases are developed in addition to immunotherapies for cancer.

### Base Editor Screening Platform in Primary Human T Cells.

Systematic discovery with nucleotide resolution of T cell gene programs allows for exploration of T cell phenotypes in depth, examine coding and non-coding regions of key loci, and defining functional domains and motifs of immune proteins.

### Advantages.

In addition to the nucleotide resolution afforded by base editing, an additional major advantage is the ability to multiplex gene modifications. Without the need to introduce double-strand breaks, base editing allows for safe and efficient multiplex genetic modifications to enhance T cell phenotypes. For example, gain-of-function and/or loss-of-function variants of different genes in one system or combining a synthetic knock-in construct at one locus with a knockout of a target gene at another can be accomplished. Base editing also allows for the identification and incorporation of variants that affect only one phenotype among the many phenotypes associated with pleiotropic immune proteins. That is, introduction of single nucleotide variants allows for synthetic separation of functions, thereby allowing precise modulation of T cell phenotypes without undesirable side effects of general gene up- or down-regulation. This level of specificity enables the detailed dissection of functional and structural modifications across an entire coding sequence—with implications for our understanding of basic immune biology—as well as for its relevance to synthetic immunology and the design of improved cellular therapies for a wide range of diseases.

Dissect Genetic Regulation of T Cell Function with Nucleotide Resolution Using Base Editing.

CRISPR activation (CRISPRa) and CRISPR interference (CRISPRi) are powerful tools for efficient perturbation of non-coding elements, with the potential to discover factors both necessary for and synthetically sufficient to modulate cellular phenotypes. Importantly, hits identified by these screens highlight both positive and negative regulators that can be incorporated into synthetic circuits to tune cytokine responses in cellular immunotherapies. However, these traditional CRISPR approaches lack the resolution to determine the consequences of individual nucleotide changes on gene and cellular function.

DNA base editors, such as cytosine base editors (CBEs) or adenine base editors (ABEs), are powerful tools to introduce point mutations with high efficiency. Thus far, base editing in human T cells has been used for knockout purposes to demonstrate therapeutic applicability via an mRNA delivery approach (Webber et al., Nat Comm, 2019 and Gaudelli et al., Nat Biotech, 2020). However, the ability to introduce single variants systematically is a powerful, untapped discovery tool. To this end, a base editor screening platform was prepared using lentiviral delivery of both CBE and ABE in primary human T cells. A sgRNA library tiling the coding regions of about 400 genes important for T cell activation was used to perform base edits with CBE or ABE within a defined 12 base pair or 5 base pair window, respectively. The unbiased nature of the library design allowed for nonsynonymous and synonymous editing, nonsense mutations as well as non-editing sgRNAs. A preliminary base editor screen of a T cell activation gene panel screened for cytokine production (IFN-gamma, IL-2, TNFa) and cell surface makers (PD-1, CD25). Variants were identified with significant effects on T cell activation that nominated functional hotspots of protein function. These screens revealed functional residues, domains, and motifs whose alteration had clear effects on T cell phenotype.

Interestingly, distinct hotspots in individual genes were identified that, when mutated, resulted in both loss-of-function and gain-of-function phenotypes, with potential to act as functional switches to tune gene expression in therapeutic settings. This approach may be extended to additional panels of genes with relevance to various T cell functions and to genes associated with immune-mediated disease to enhance and improve next-generation adoptive cellular therapies. Base editing technology allows for the exploration of T cell phenotypes in depth, examination of coding and non-coding regions of key loci, and definition of functional domains and motifs of immune proteins. Many proteins have multiple independently and post-translationally regulated functions, such as signaling, scaffolding, protein-protein interactions, or localization, which cannot be dissected with whole-gene or up/down gene regulation approaches. Variants modifying key T cell phenotypes—including cell surface protein expression, cytokine production, and proliferation—may further interrogated using Perturbseq (Dixit et al., Cell, 2016; Adamson et al., Cell, 2016; Jaitin et al., Cell, 2016) to understand the overall effect of these variants on downstream cell state and nominate promising candidates that rewire immune cell states in advantageous directions.

### Employ Base Editing to Dissect the Genetic Architecture of Autoimmune Disease Variants.

T cell-specific and stimulus-responsive programs are governed by gene regulatory circuits composed of trans-regulators (transcription factors and epigenetic regulators) and networks of cis-regulatory elements and target genes. Core T cell gene regulatory programs are disrupted in autoimmune diseases. The majority of common genetic variants linked to human autoimmune diseases map to putative cis-regulatory elements active in human T cells (Farh et al., Nature, 2015). Putative enhancers across a range of human immune cell populations have been identified, uncovering stimulation responsive elements containing human autoimmunity-associated variants (Calderon et al., Nat Genet, 2019). CRISPR, CRISPRa, and CRISPRi have been used to functionally map and characterize enhancers, including enhancers harboring common autoimmunity variants (Simeonov et al., Nature, 2017; Mowery et al., Nat Genet, Under Review; Simeonov et al., BioRxiv). Together, these studies point to elements critical for T cell function. Application of the base editor screens in primary human T cells provide an unparalleled opportunity to identify causal nucleotide variants in autoimmune disorders.

### Next Generation Cellular Immunotherapies.

The power of gene editing technologies may be used to decode and rewrite the human immune system. Human immune cells, especially T cells, can be genetically engineered to target various cancers as well as to provide suppressive cues in the setting of autoimmunity. Despite their utility for the treatment of hematological malignancies, chimeric antigen receptor (CAR) T cells have significant limitations, which are especially clear in the context of solid tumors. Mounting evidence suggests that for many clinical applications, engineering the T cell to recognize the target cancer cells may not be sufficient to cure disease. Major challenges to the utility of immunotherapy in cancer include ensuring infiltration of the immune cells into the tumor, overcoming immunosuppressive tumor environment, and maintaining the therapeutic cells' functionality in the tumor over time. Next-generation cellular immunotherapies employ genetic engineering to produce the enhanced functionality necessary to overcome these challenges. Using the gene targets and variants identified by the discovery-based screens, a dictionary of genetic modifications can be prepared that can be rewritten into cells to precisely control their function, whether that be trafficking, cytokine production, killing capacity, persistence in patients, and/or influence on the local tissue microenvironments.

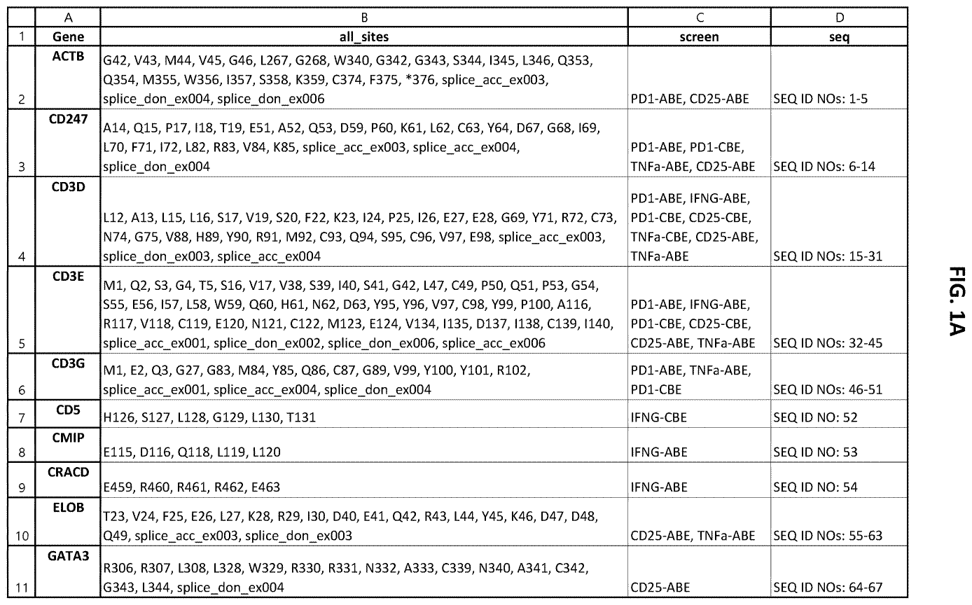

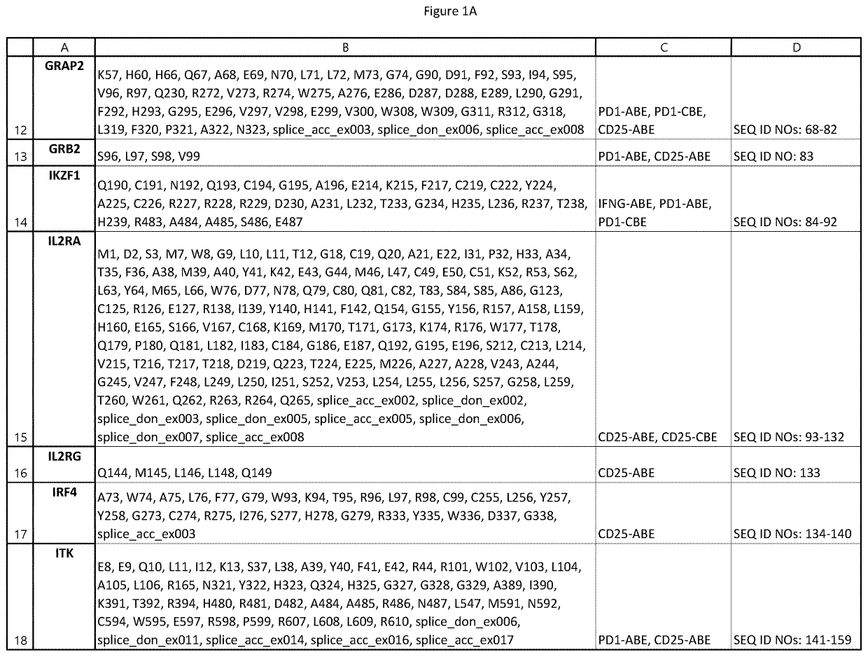

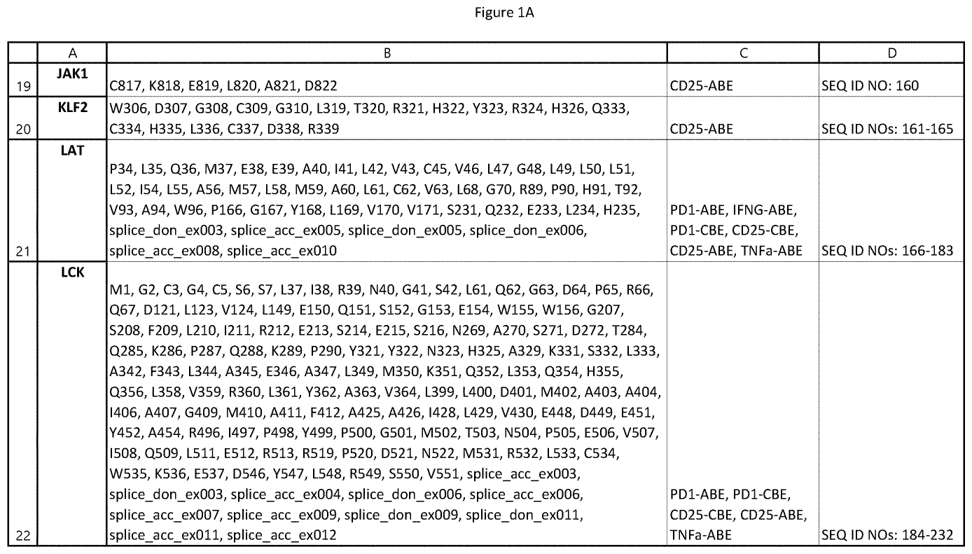

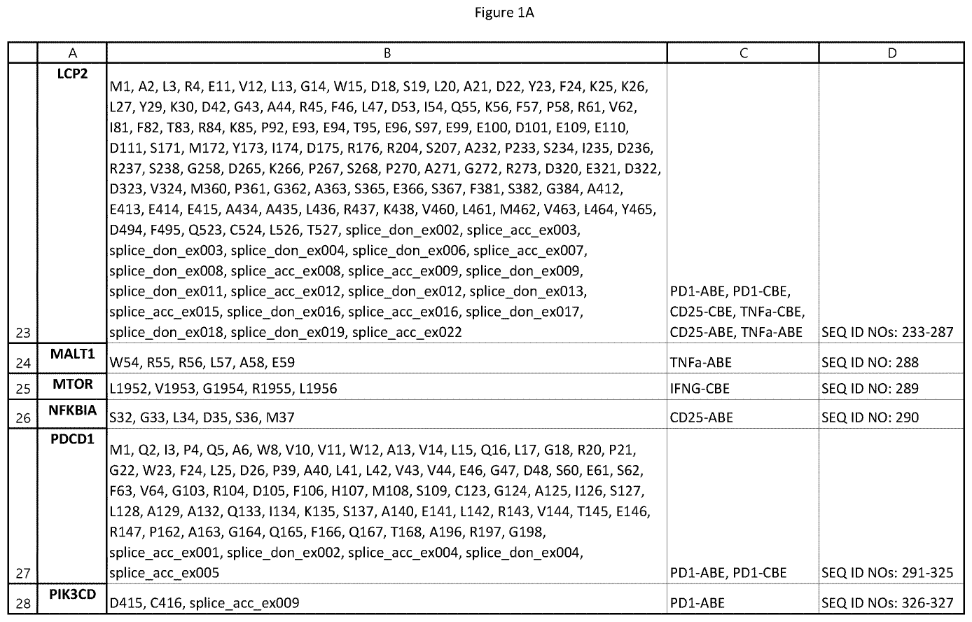

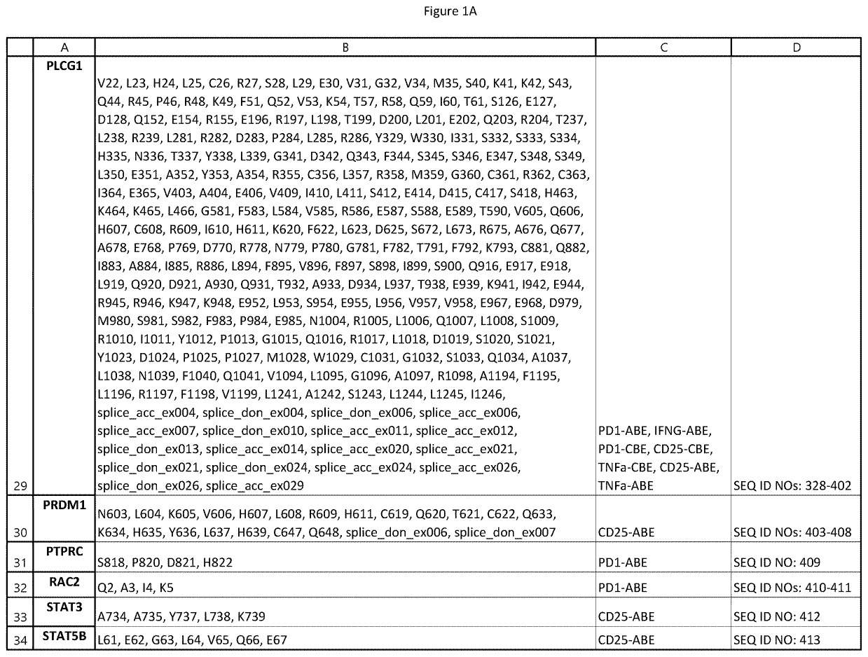

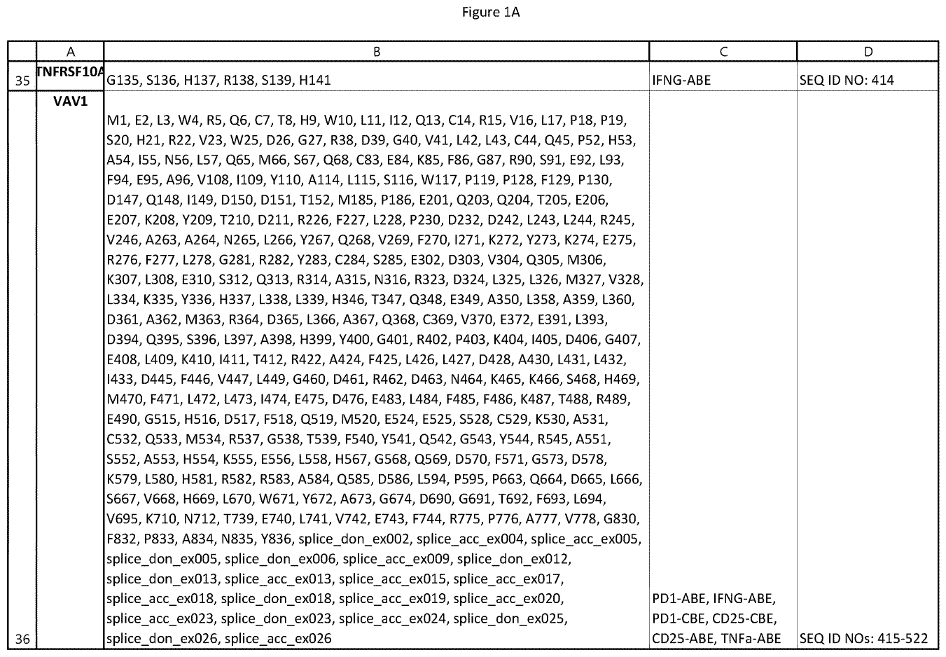

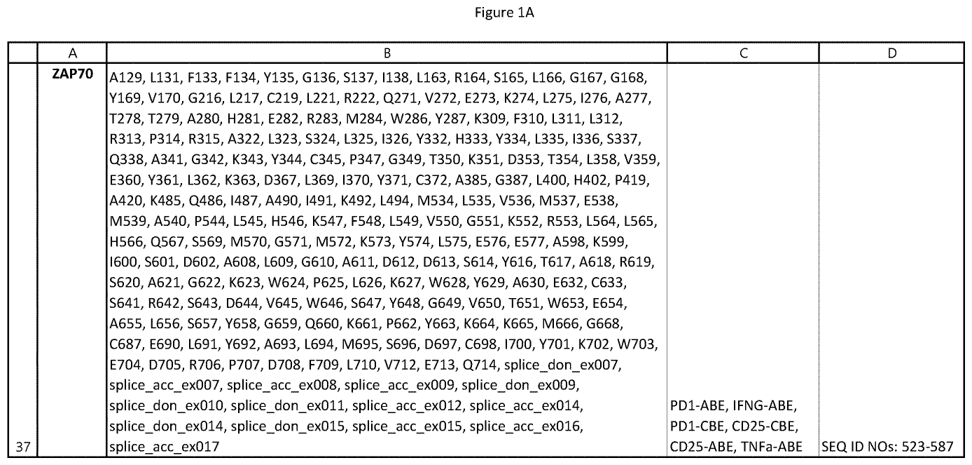

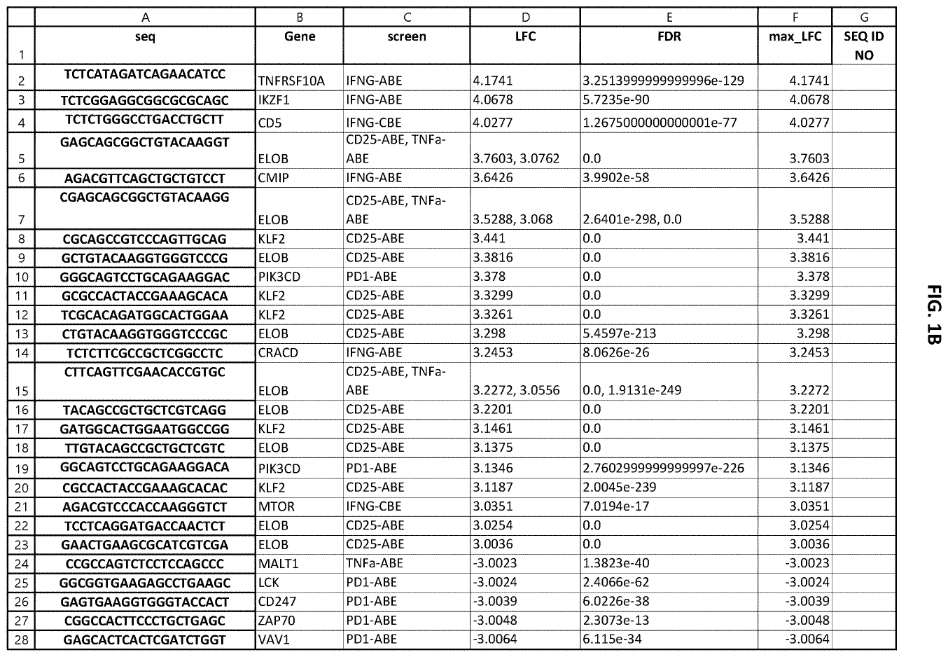

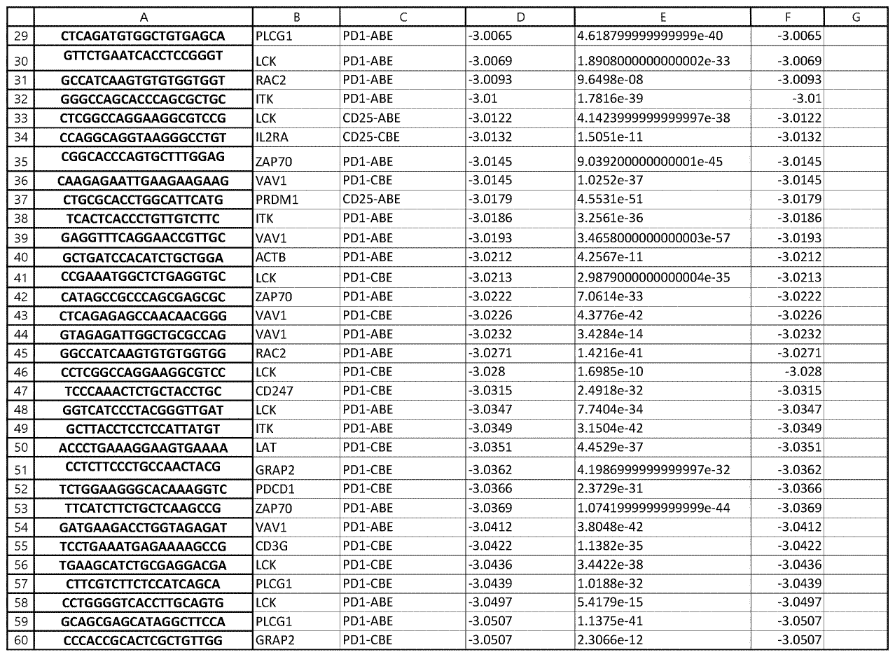

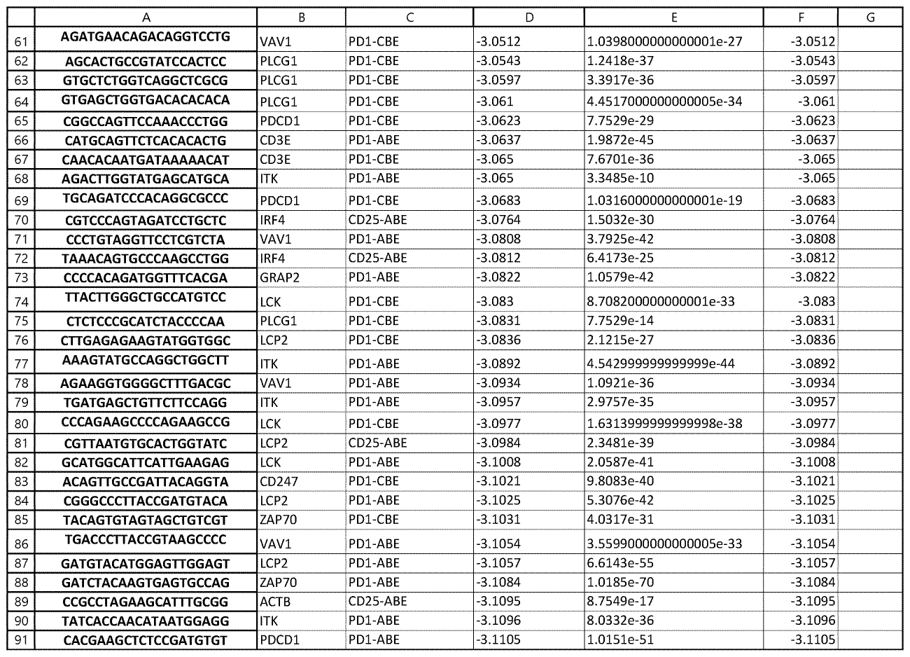

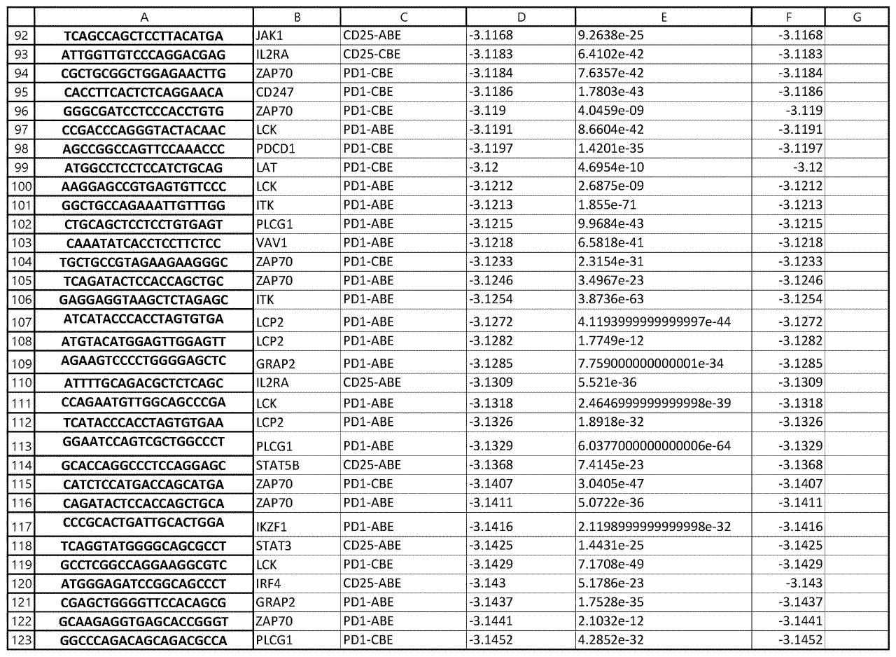

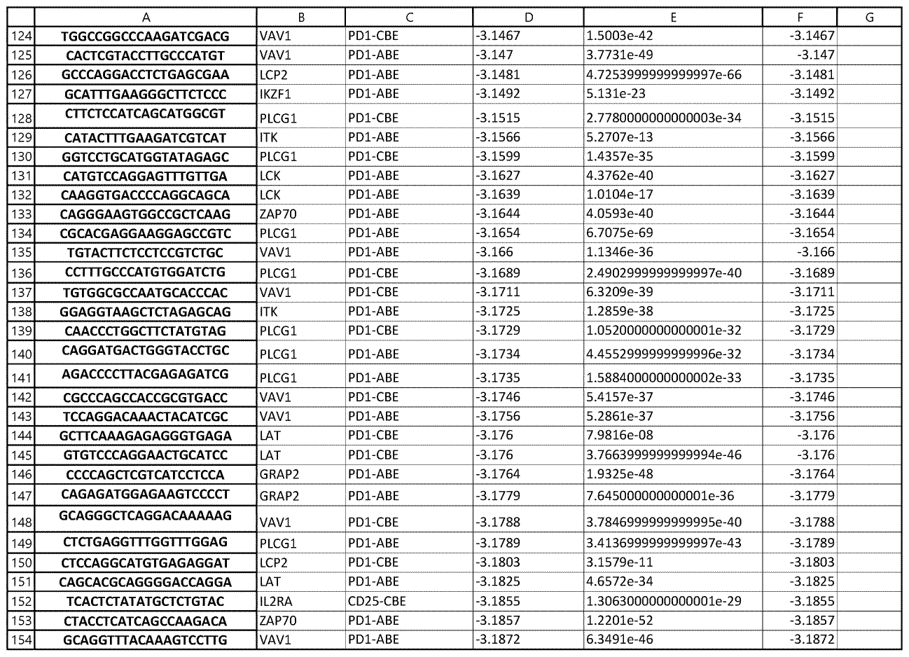

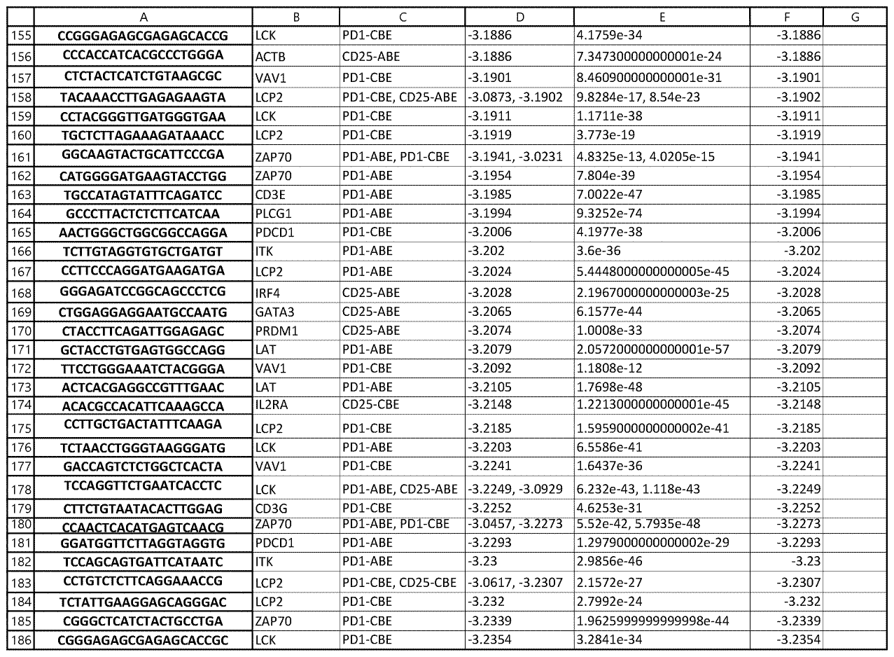

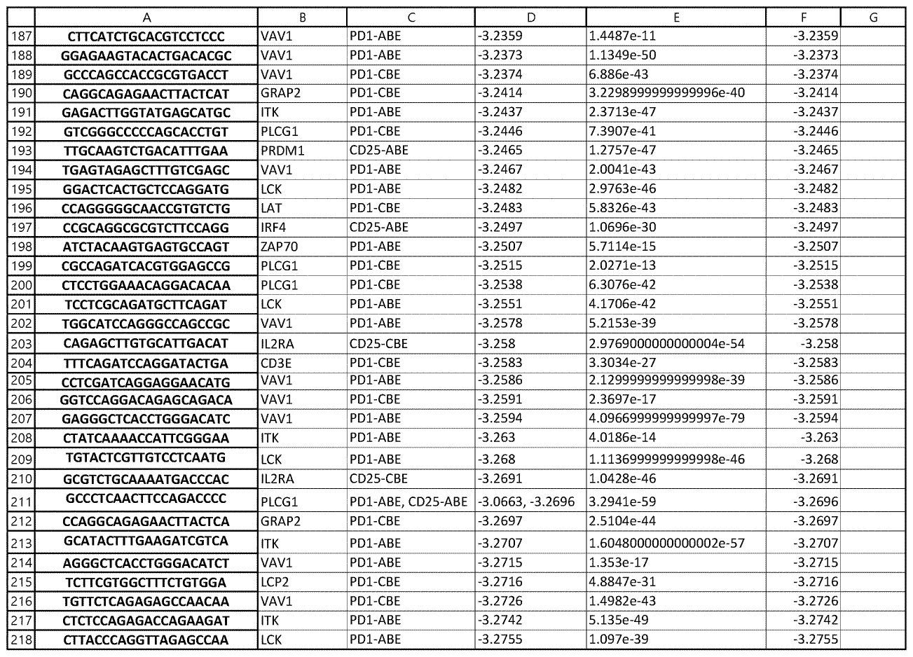

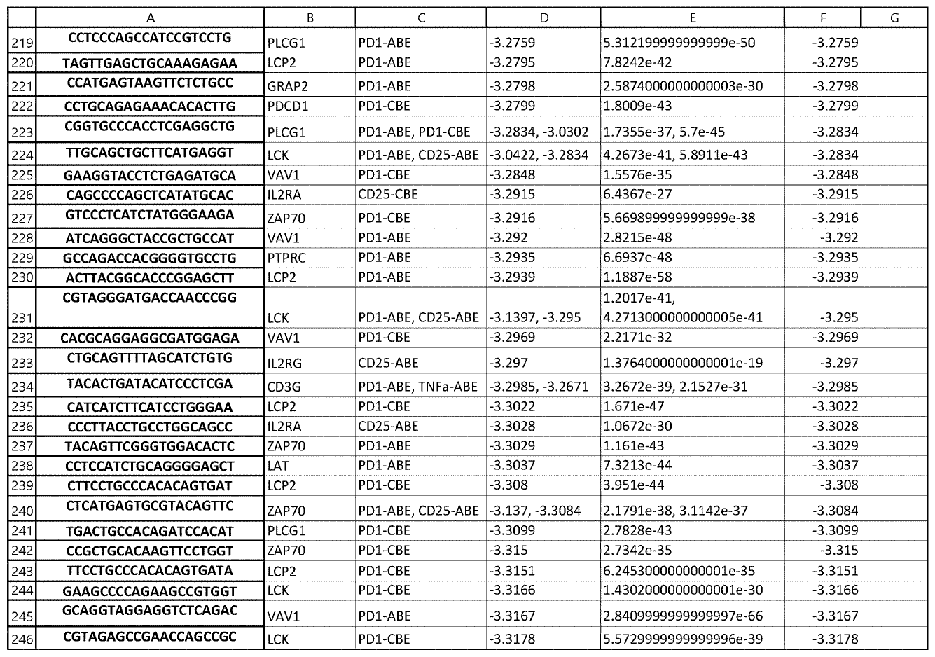

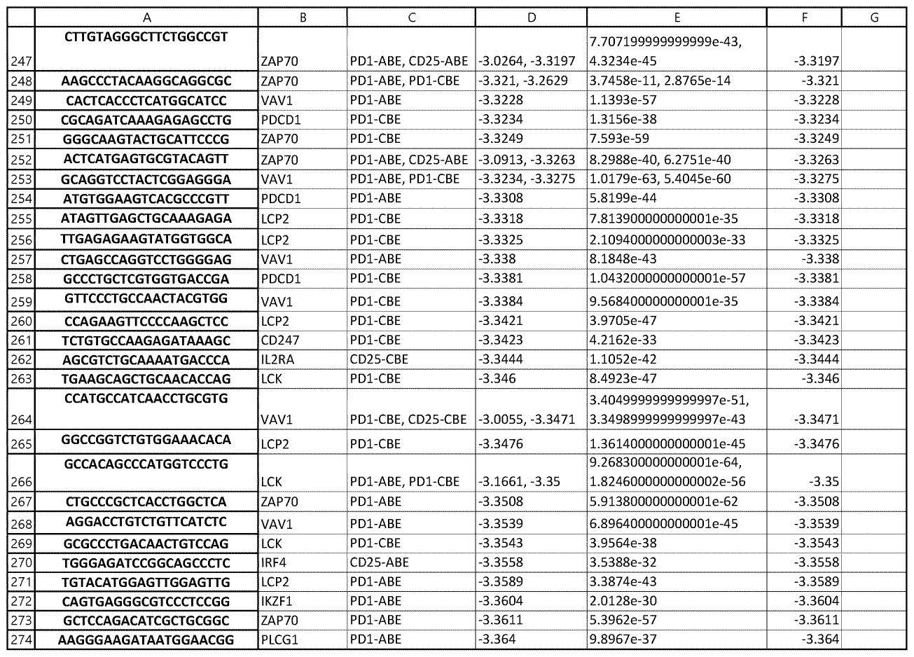

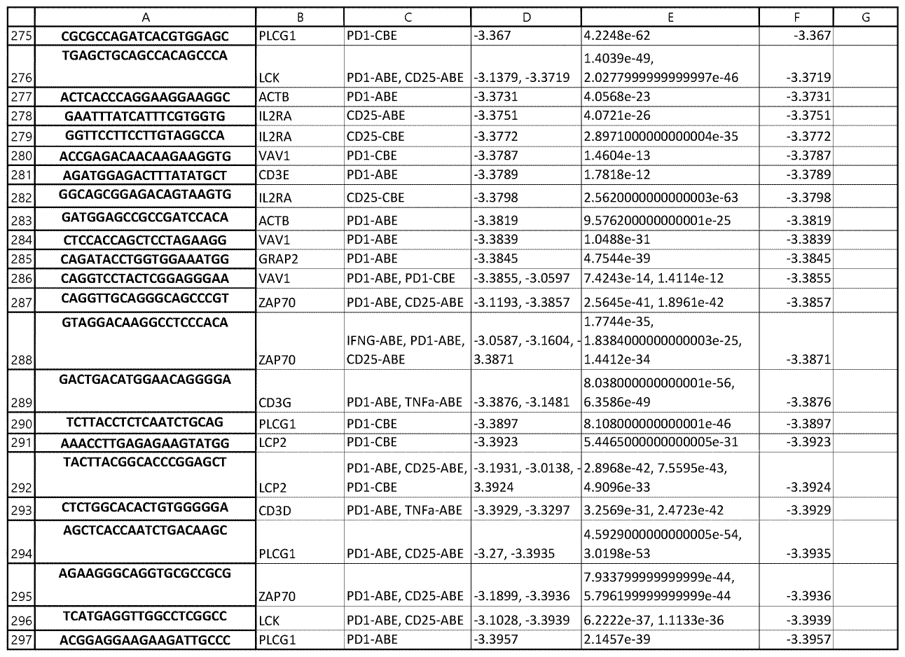

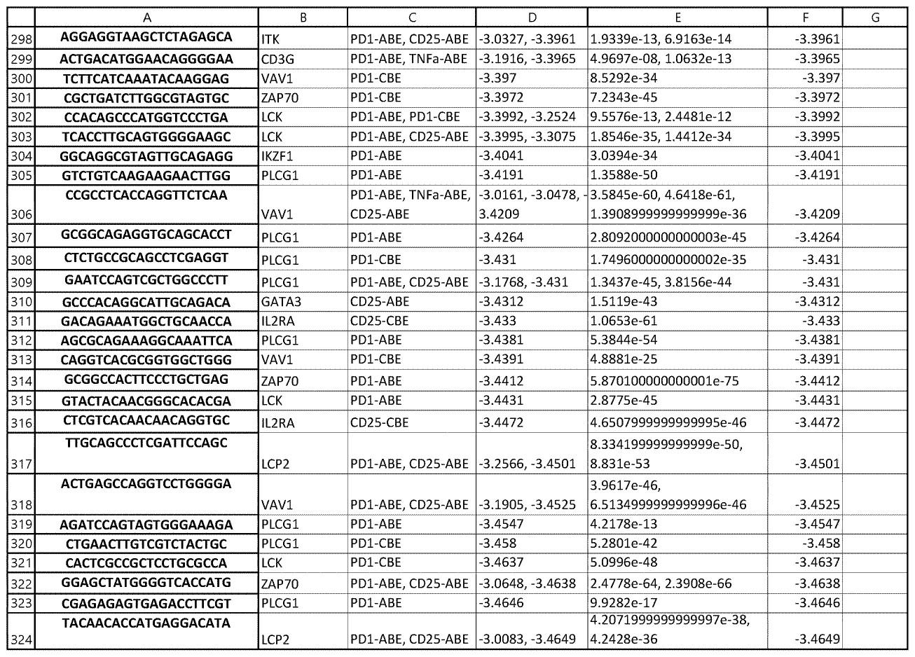

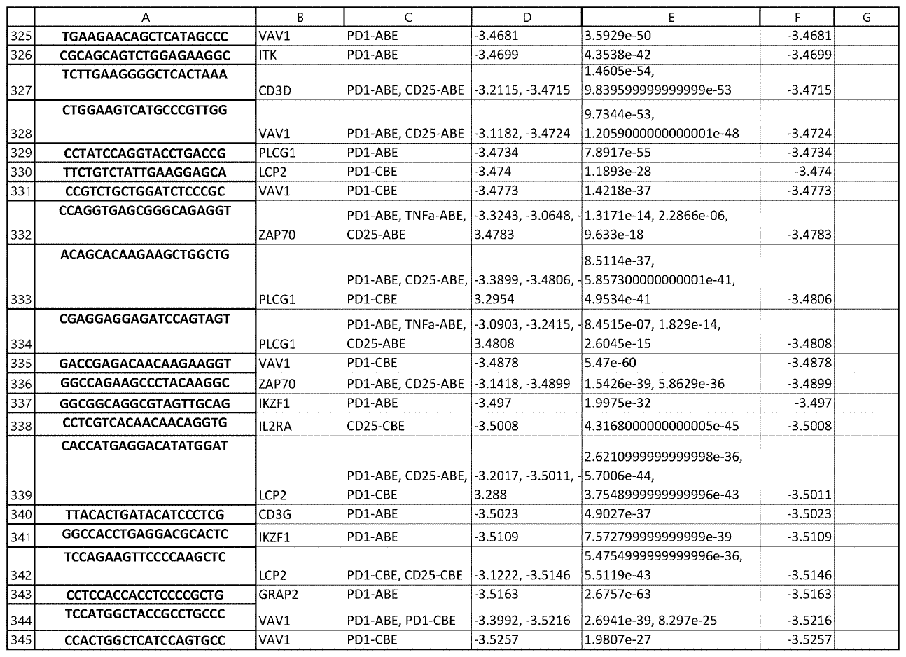

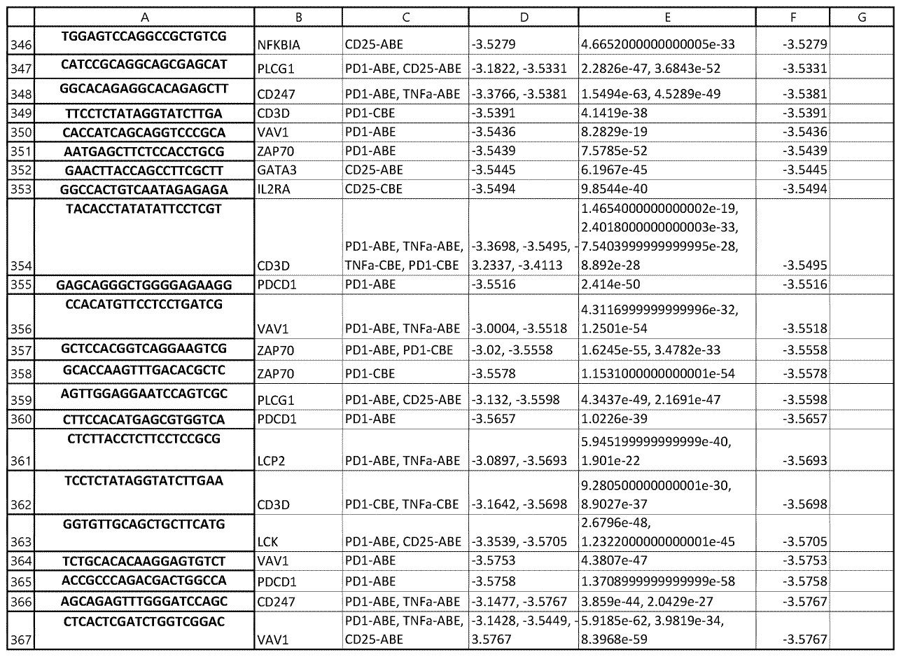

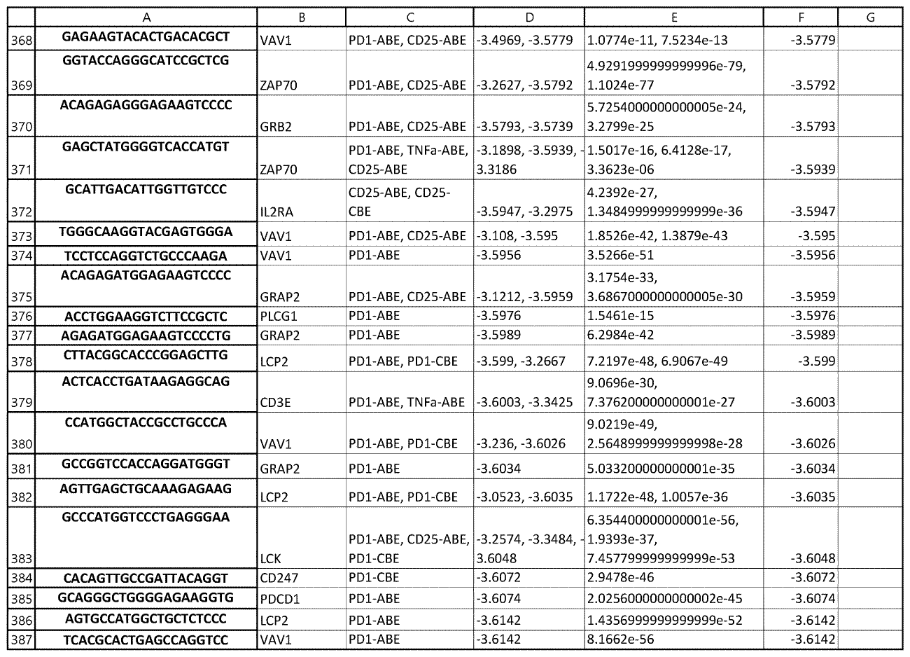

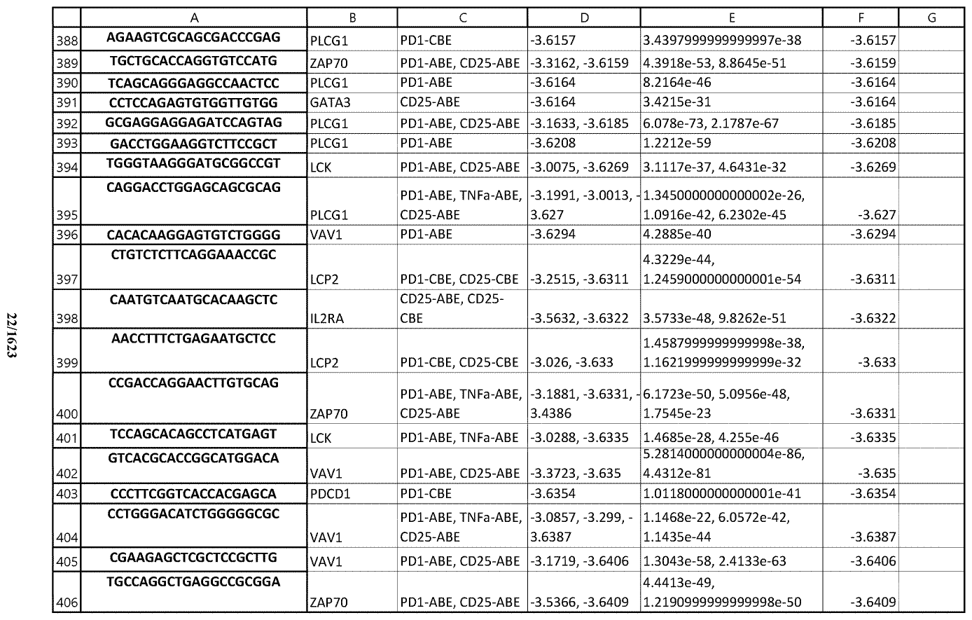

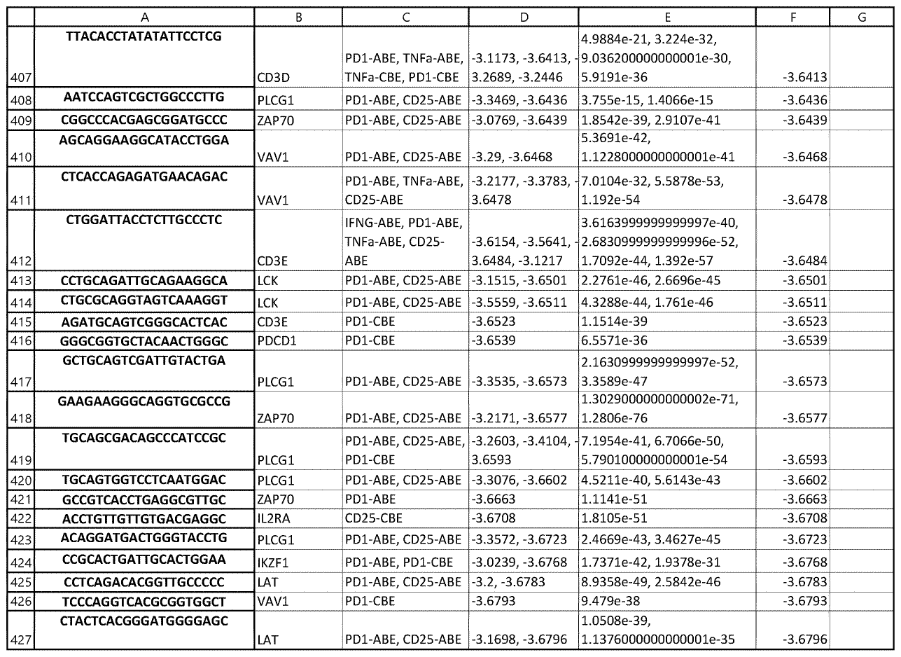

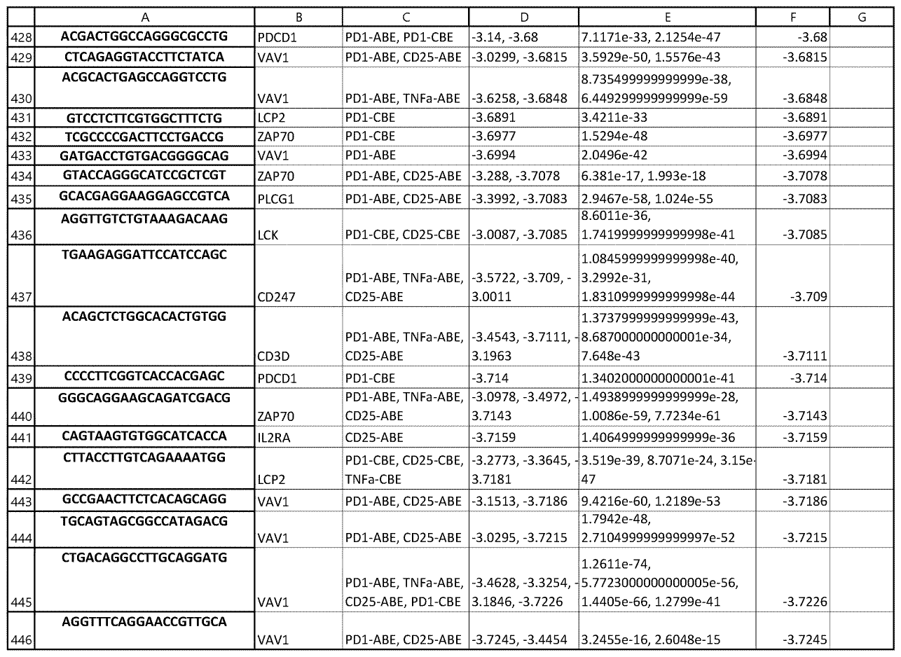

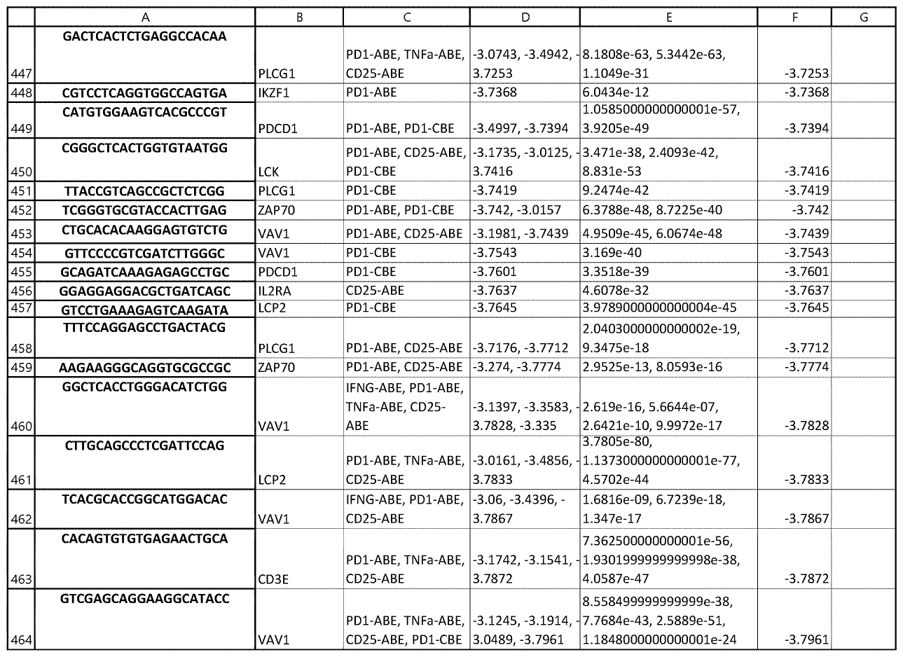

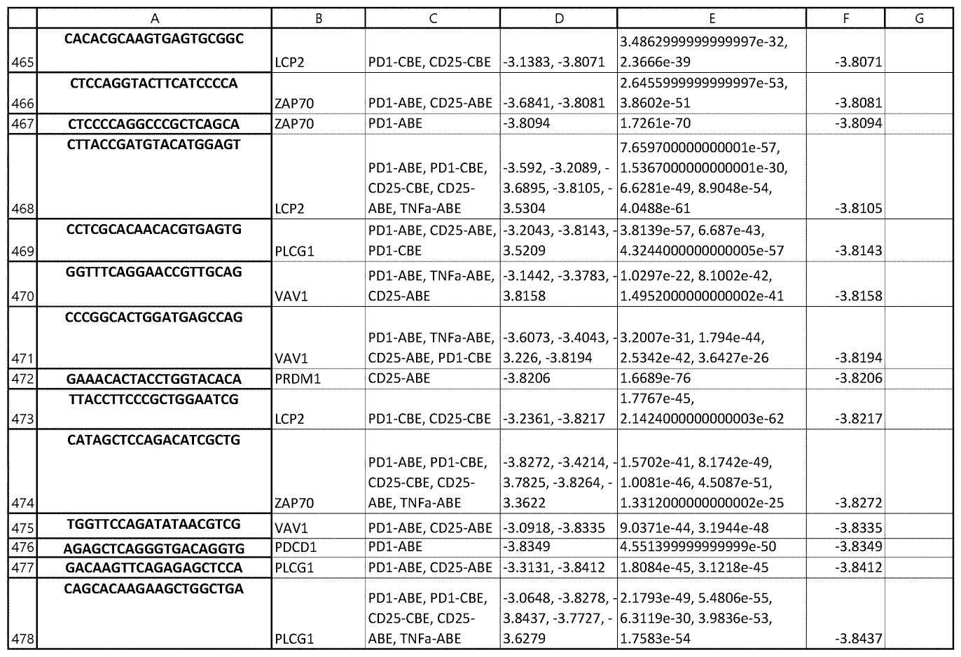

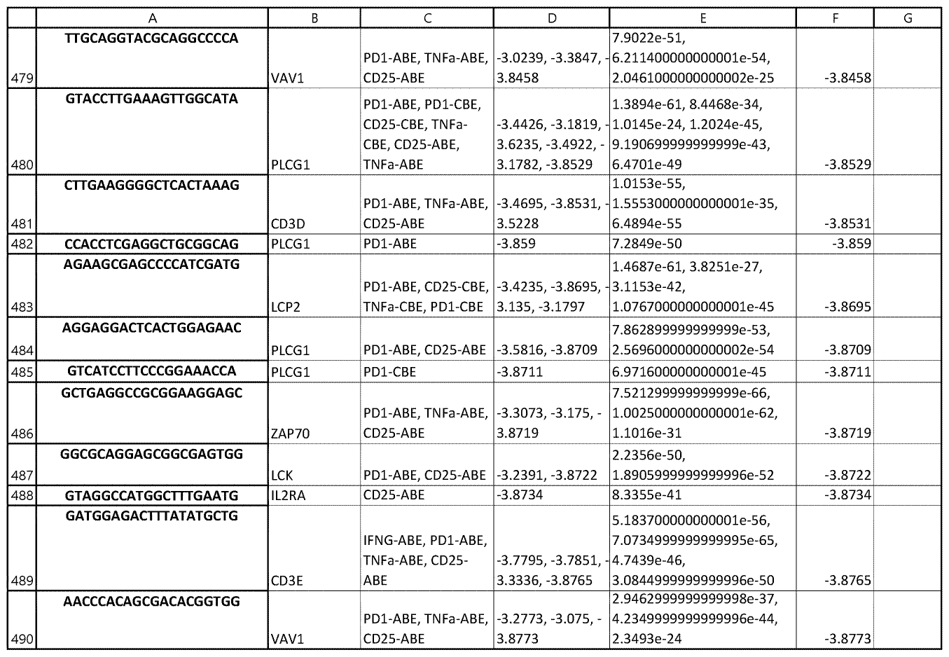

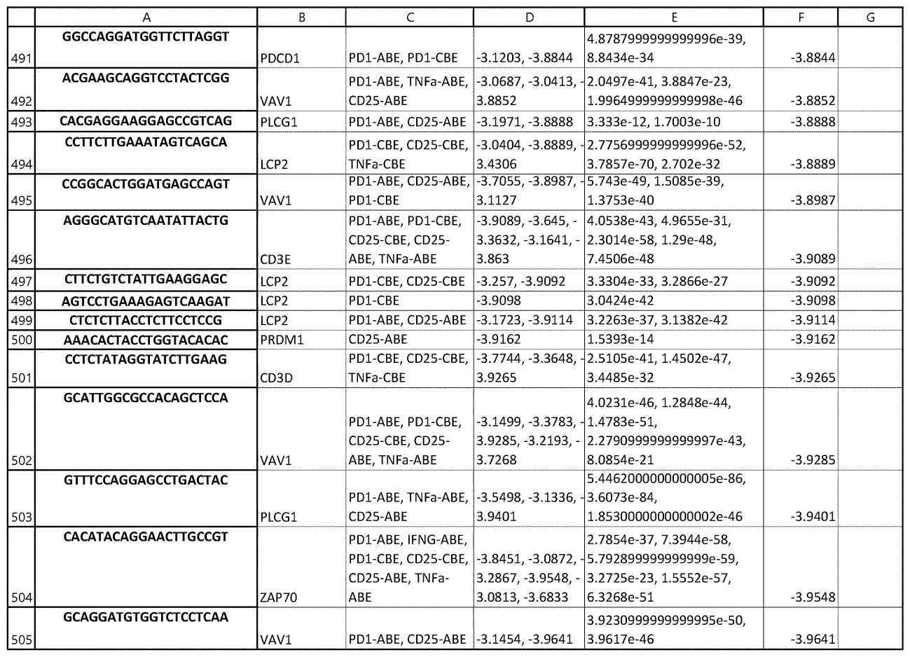

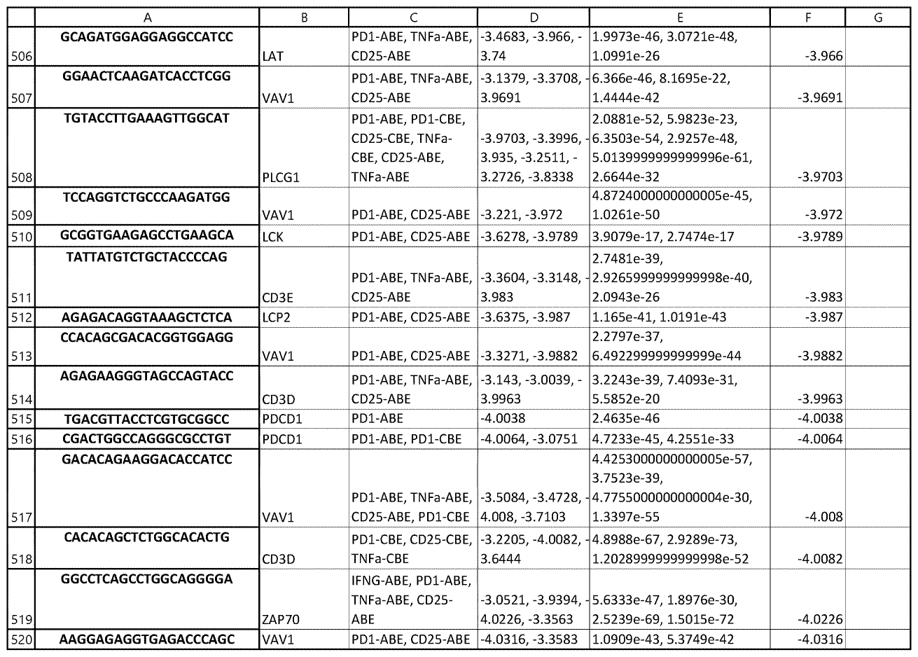

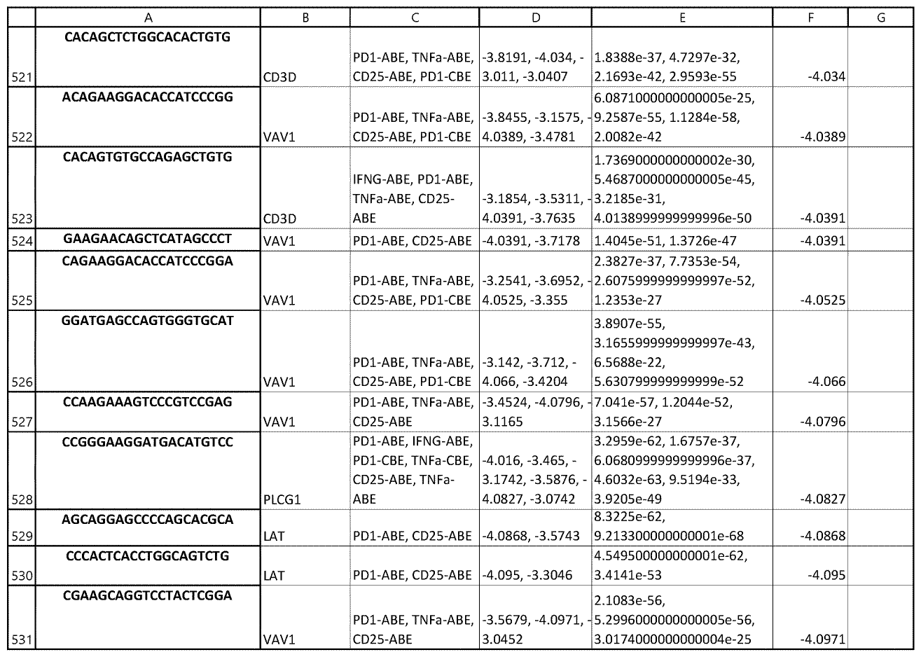

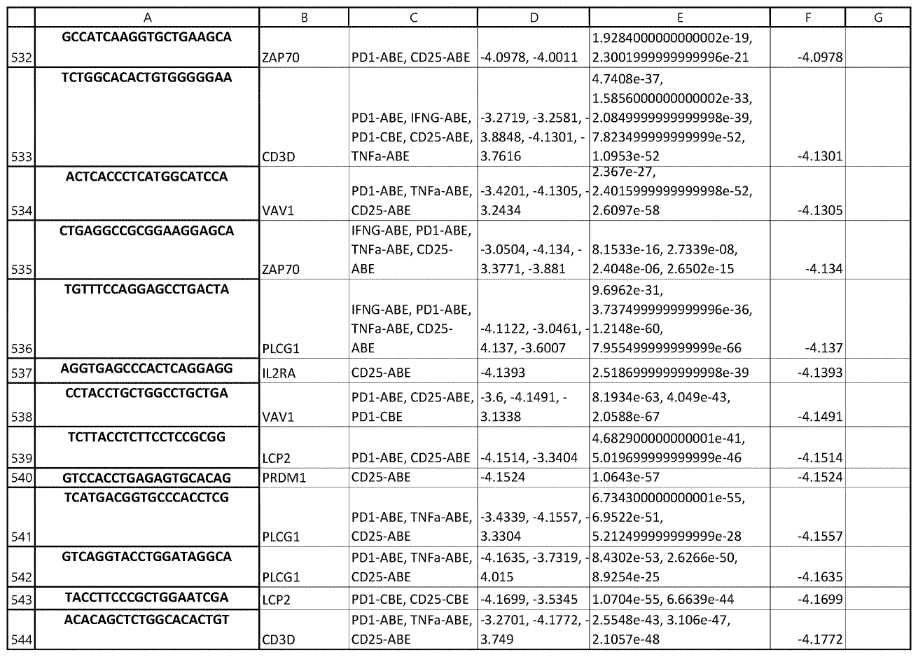

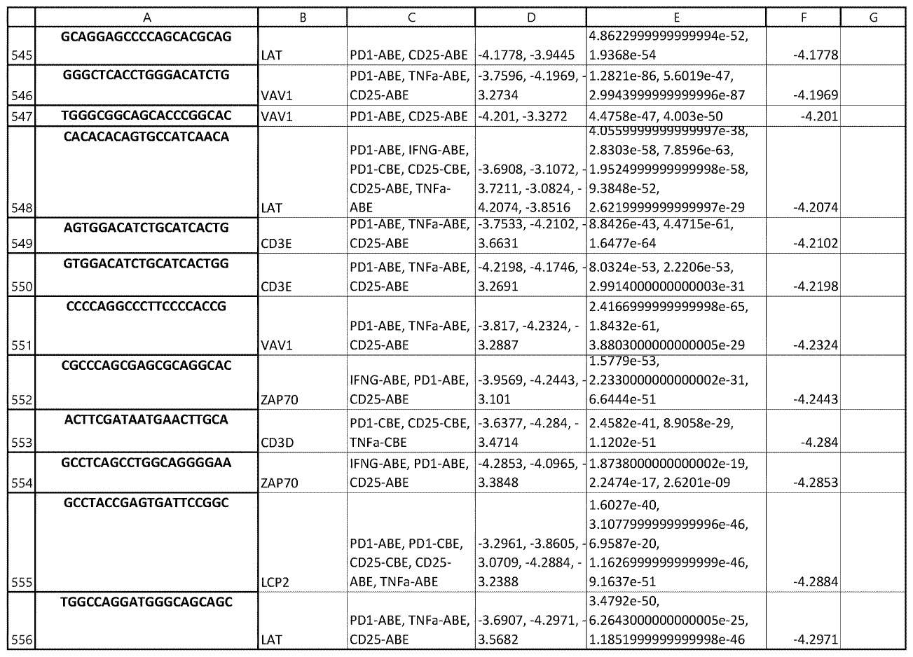

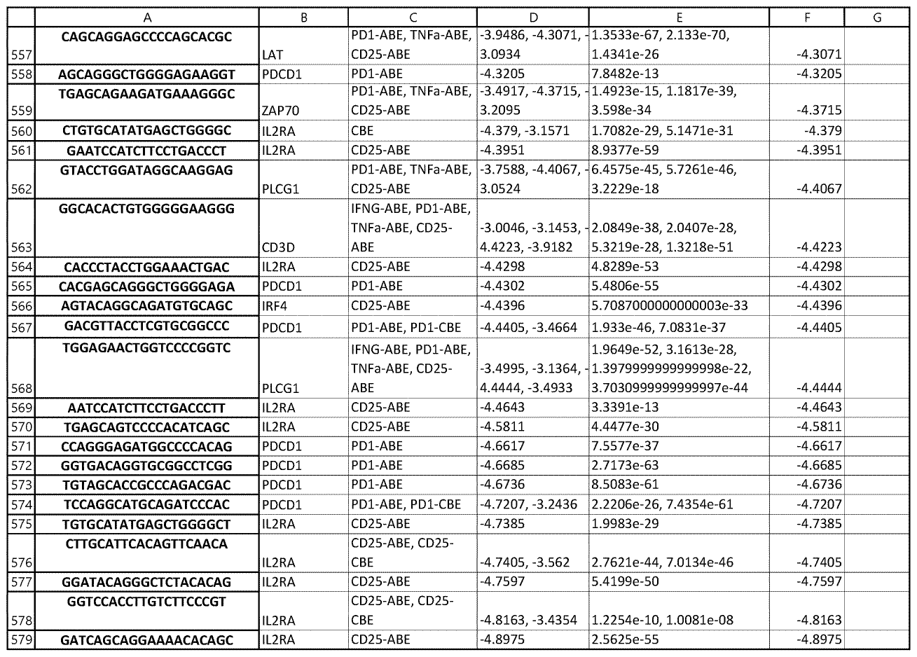

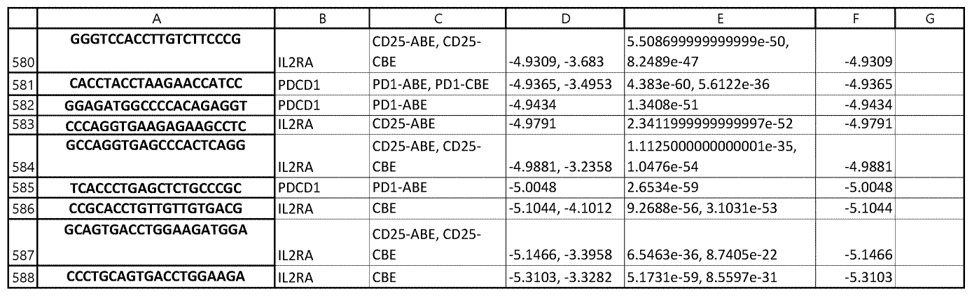

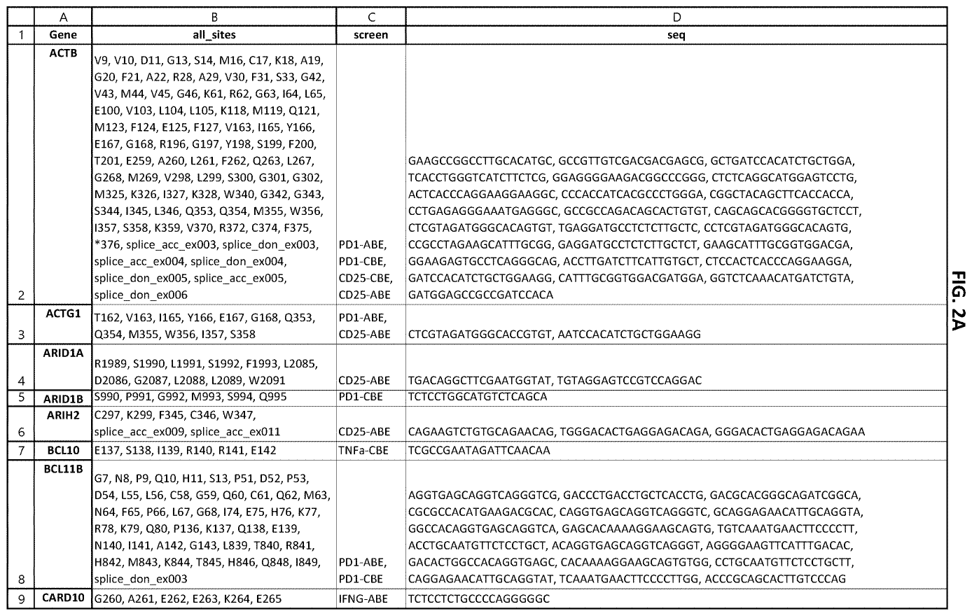

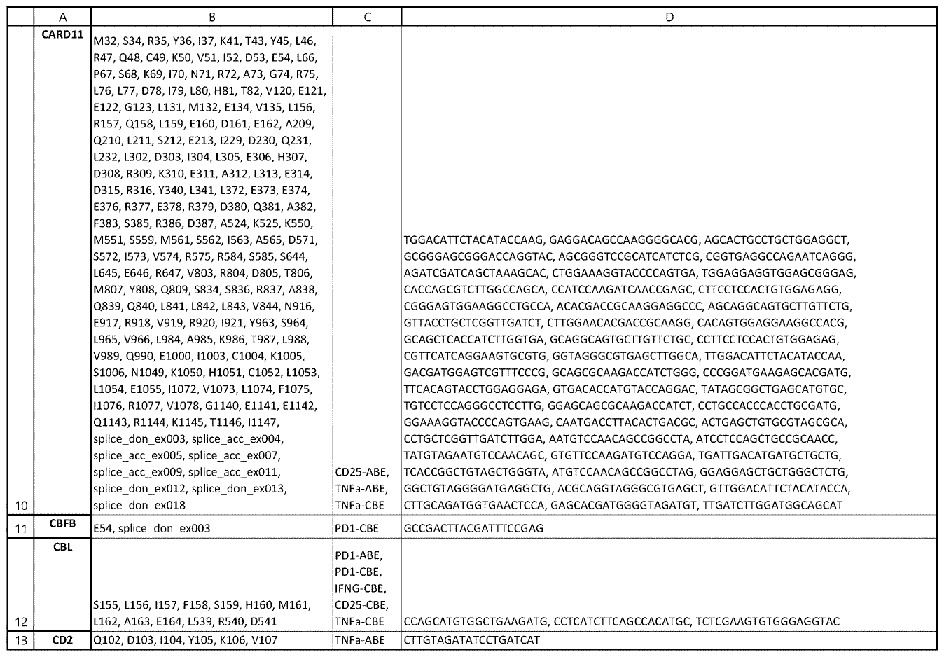

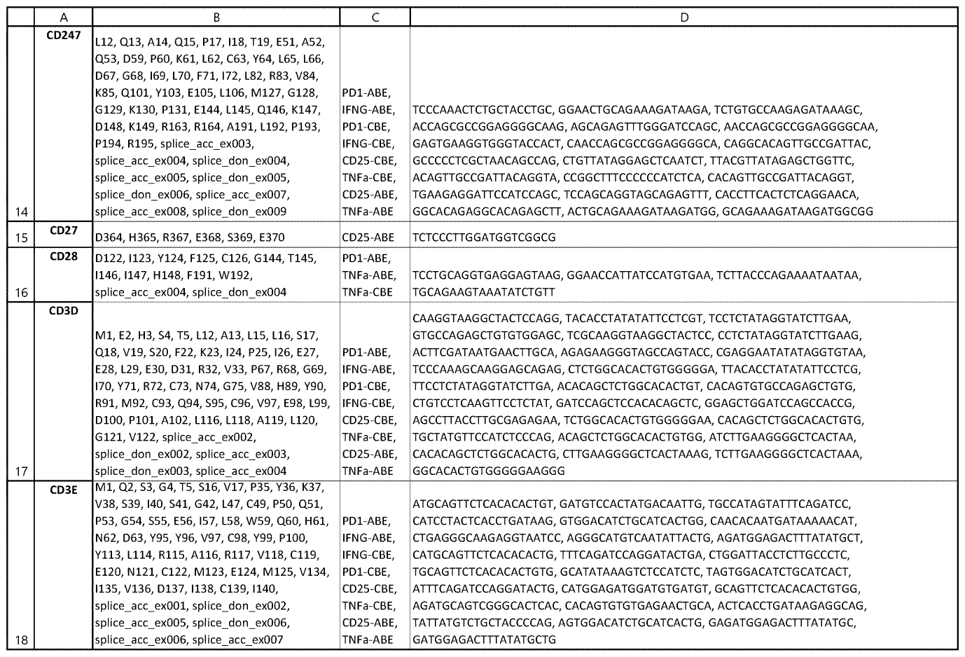

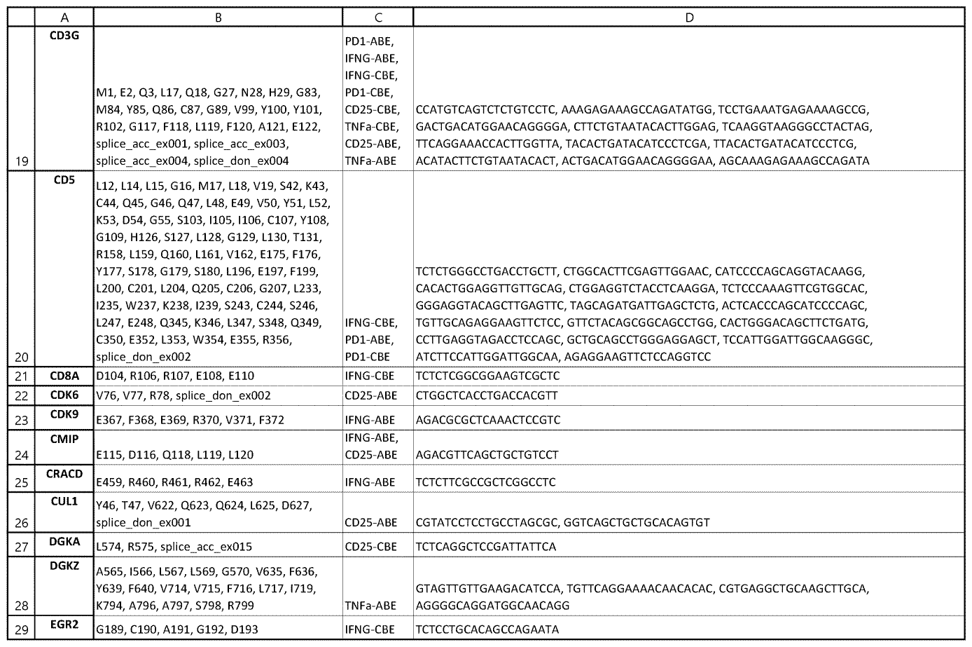

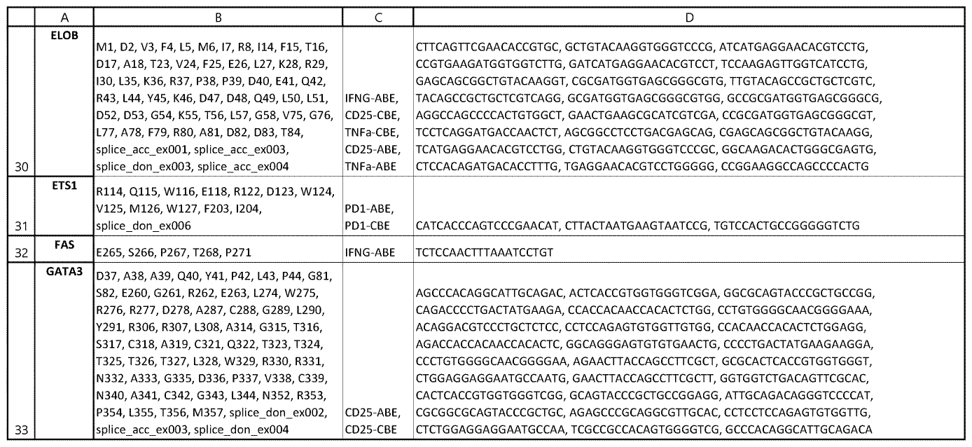

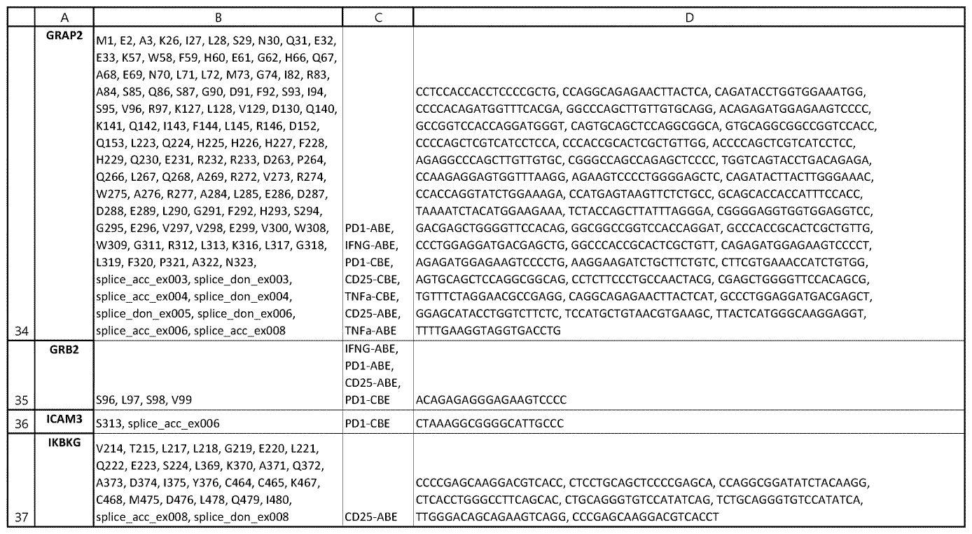

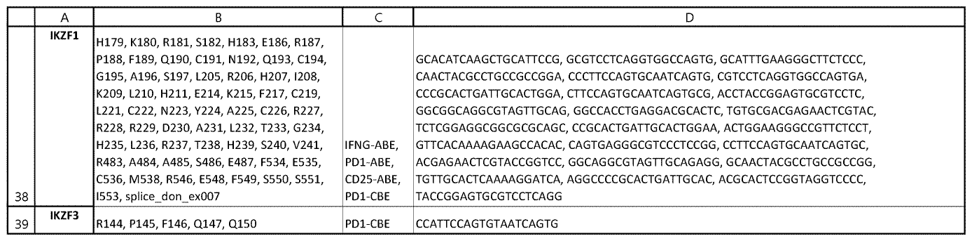

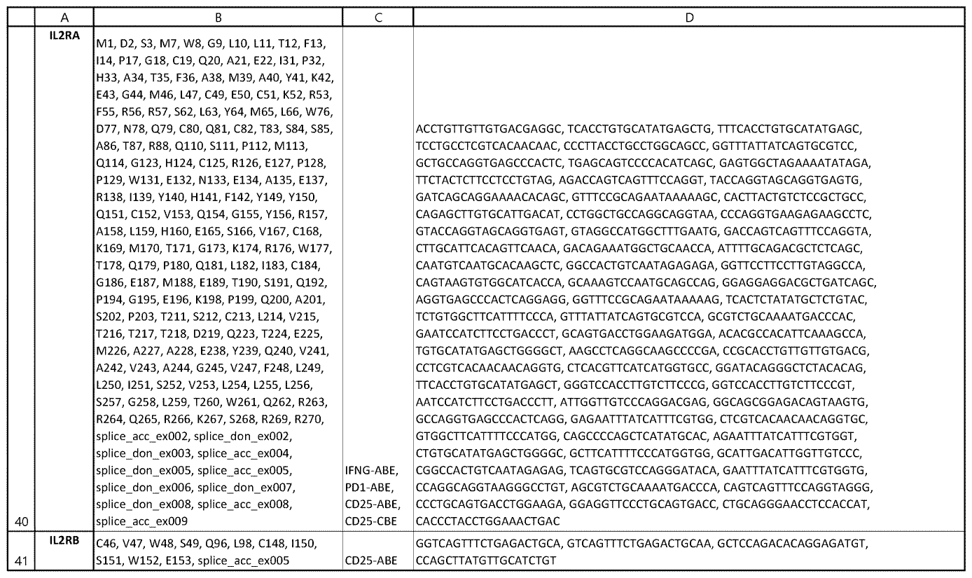

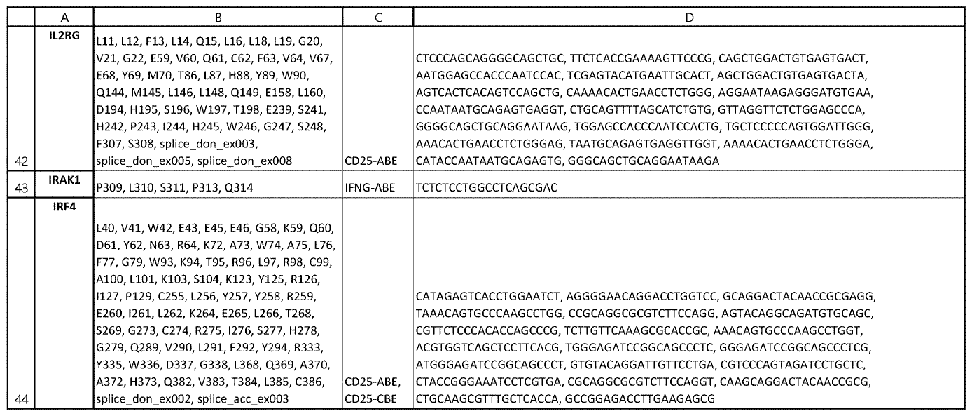

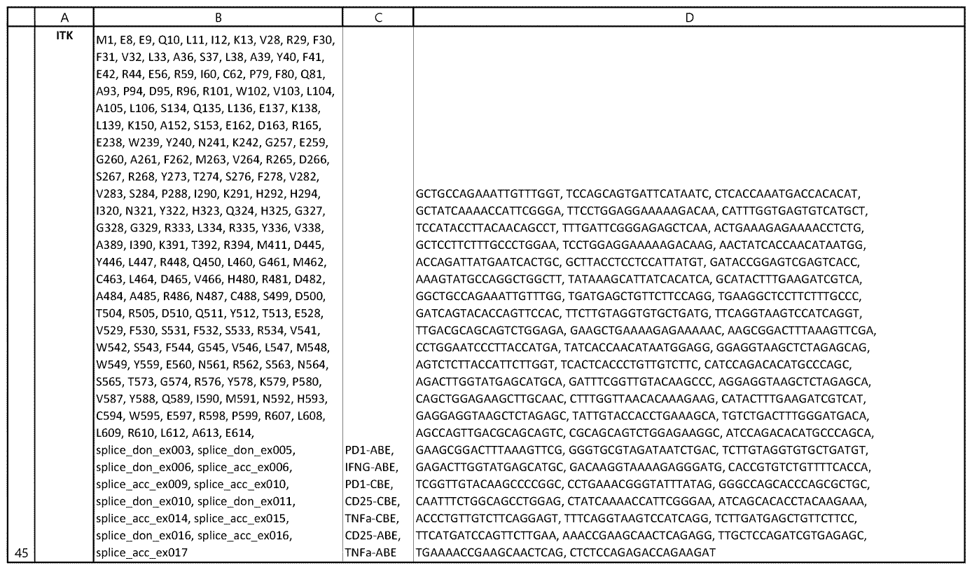

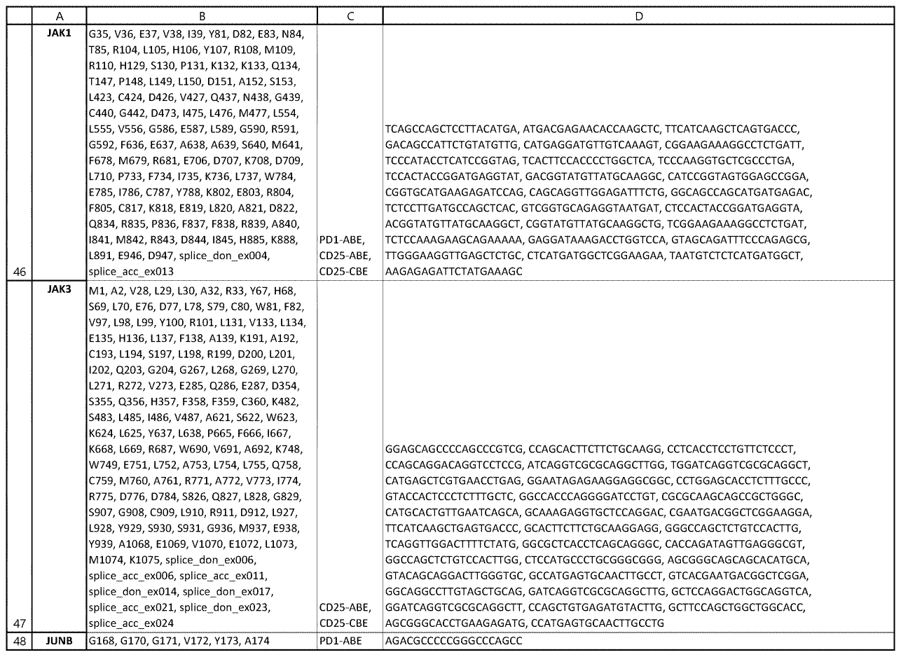

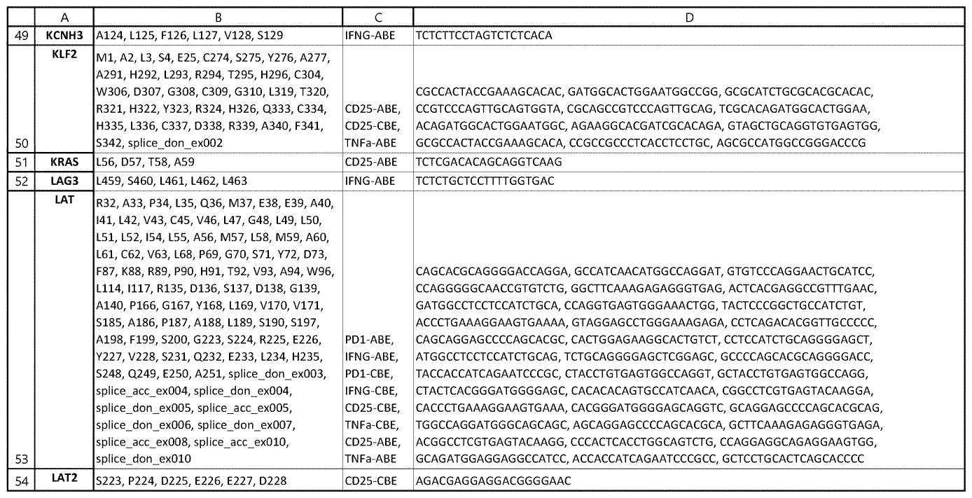

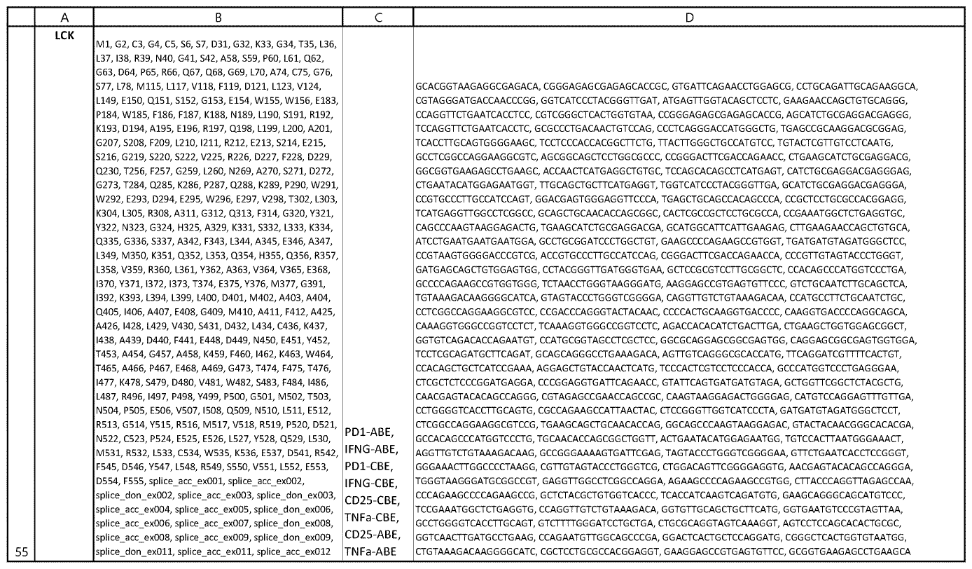

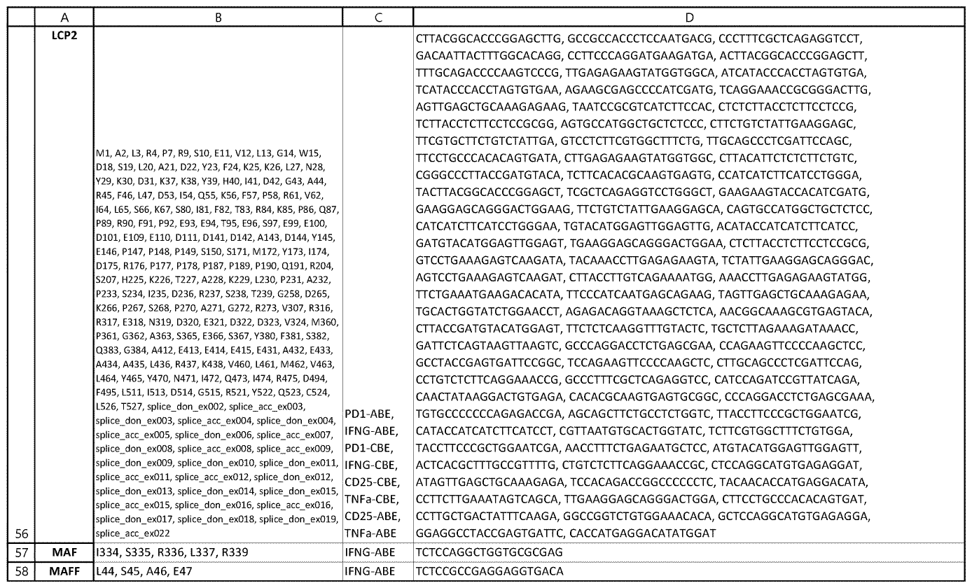

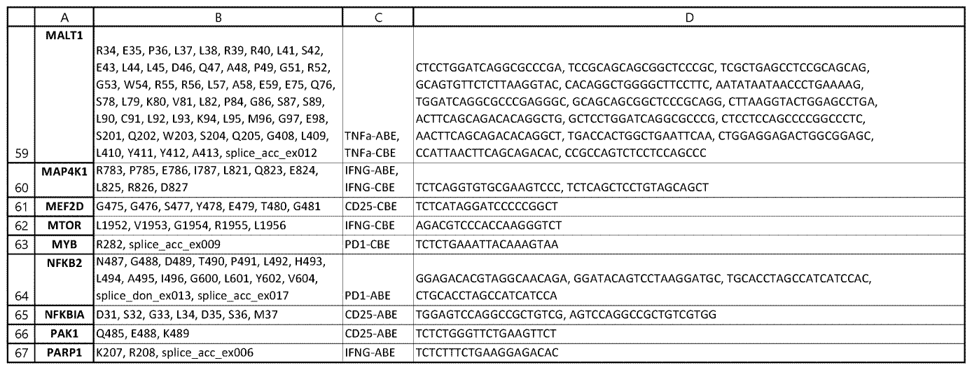

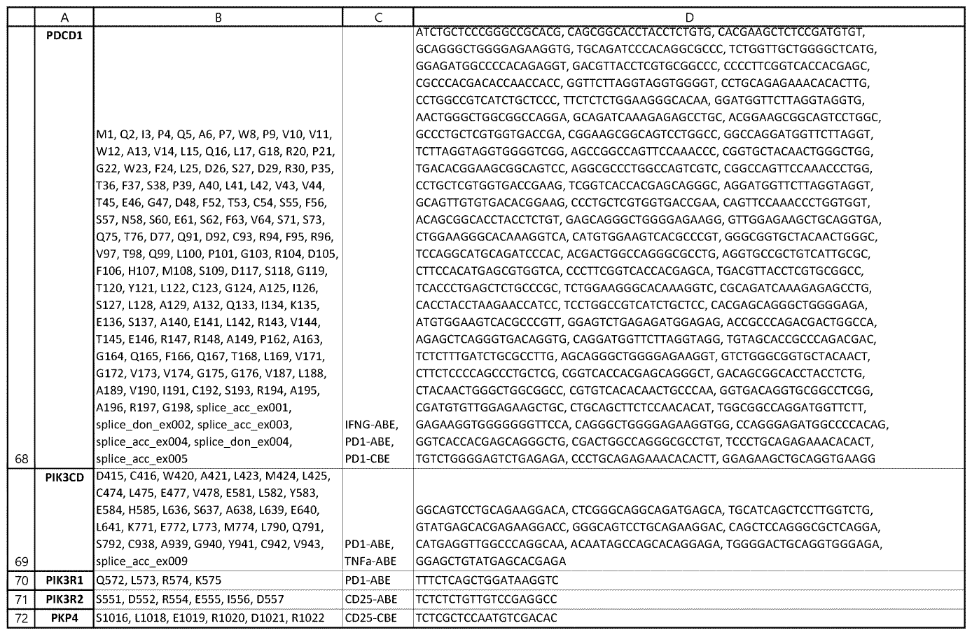

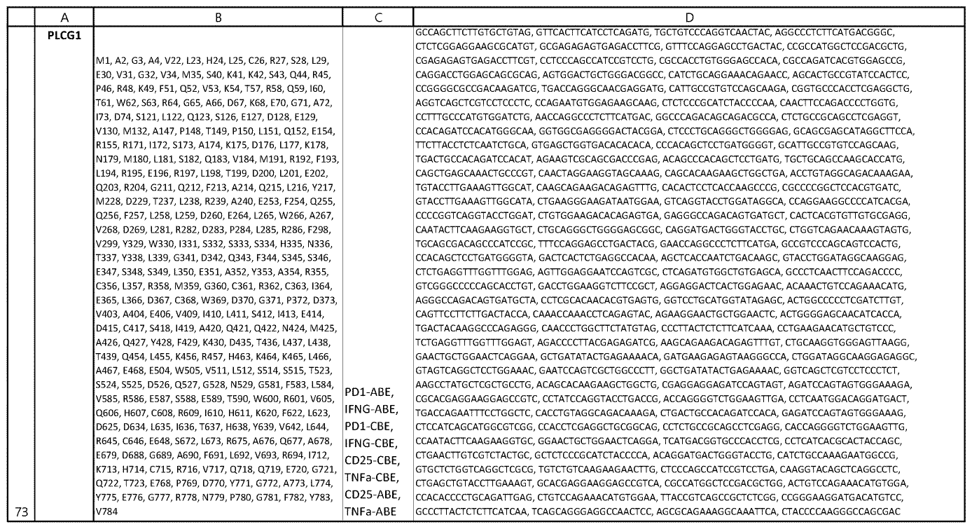

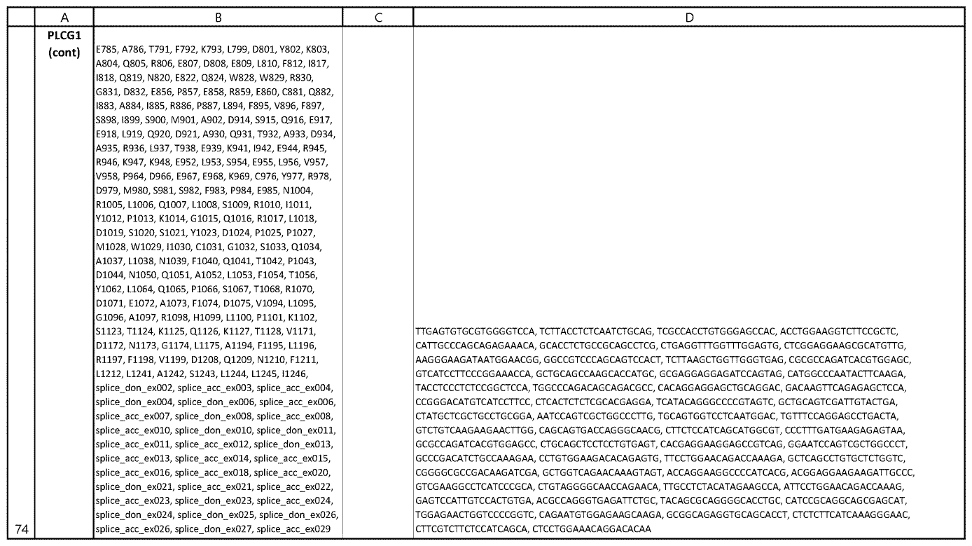

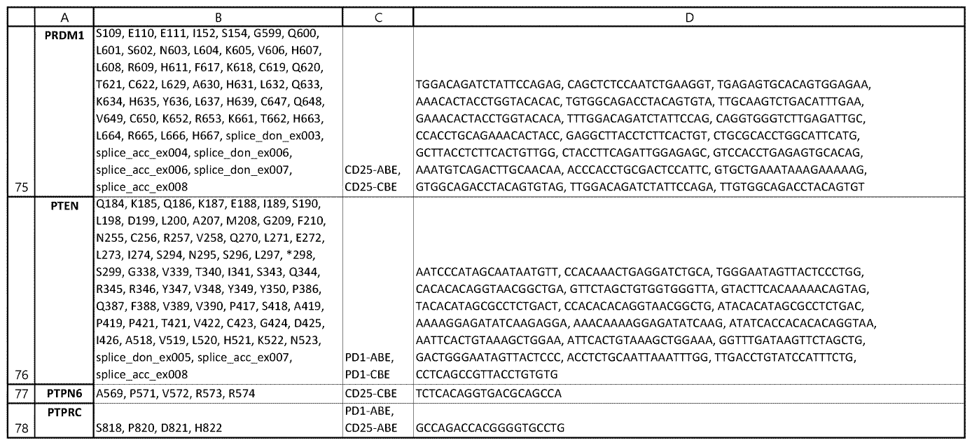

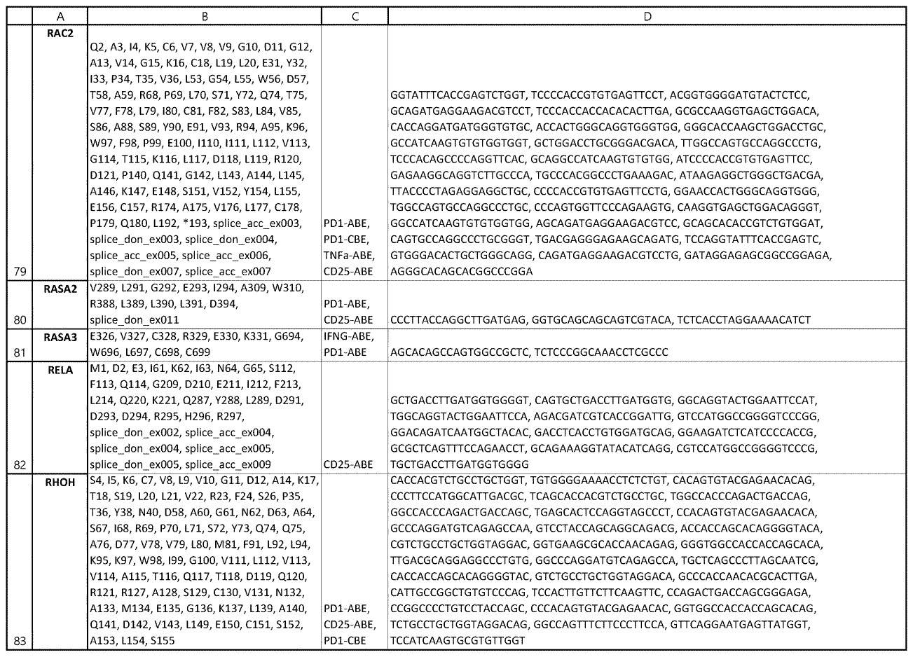

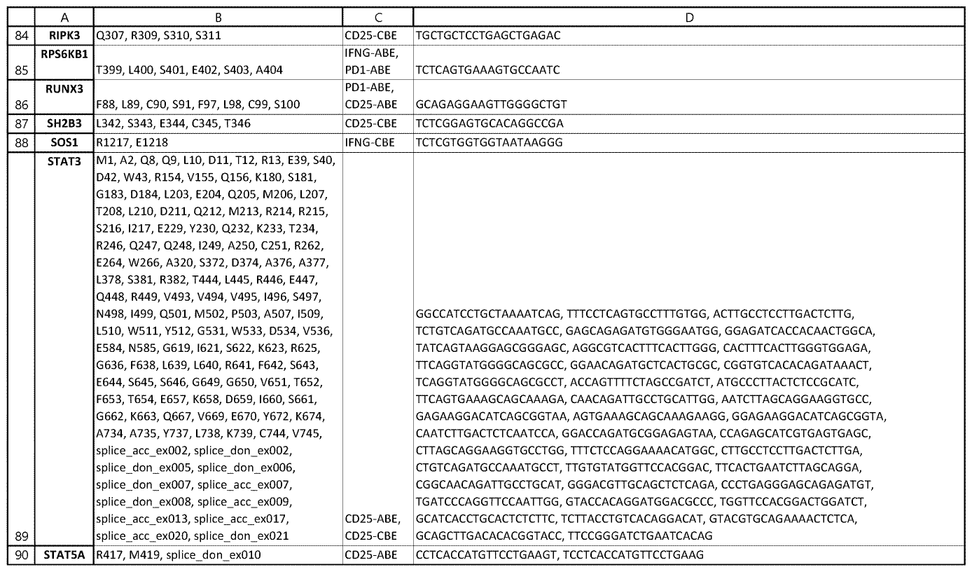

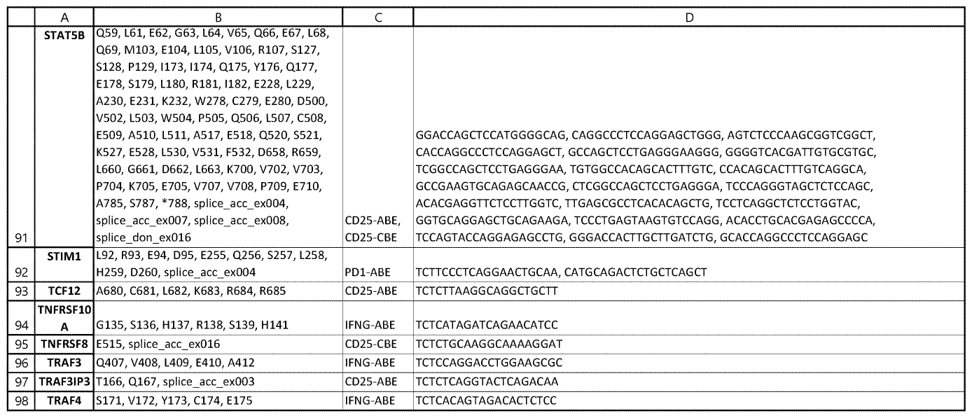

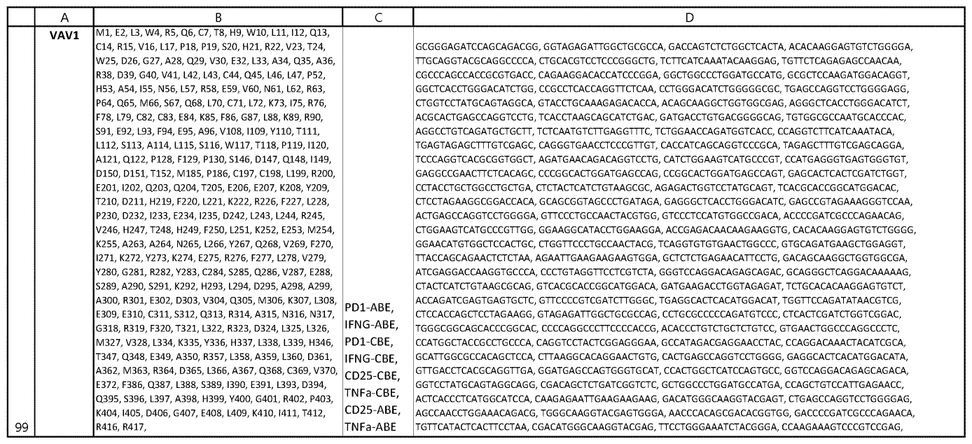

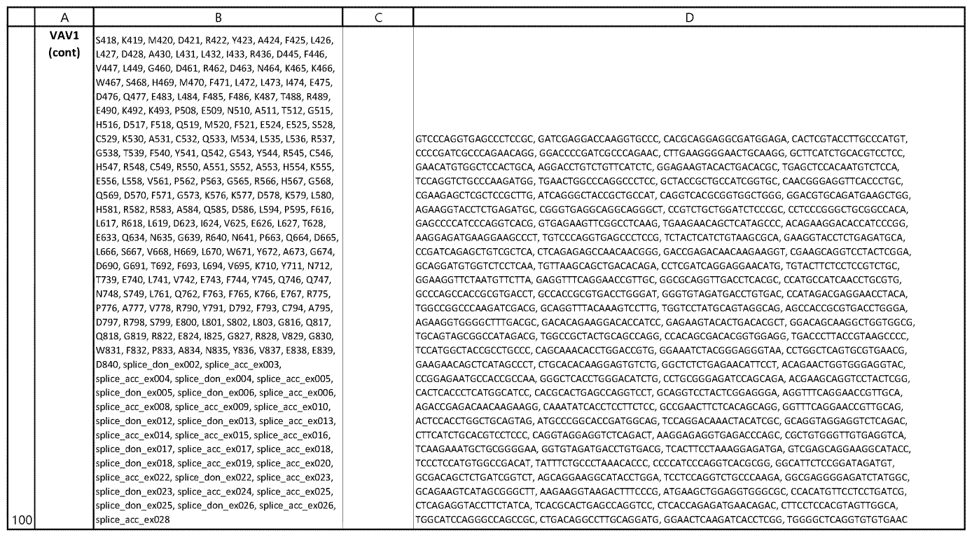

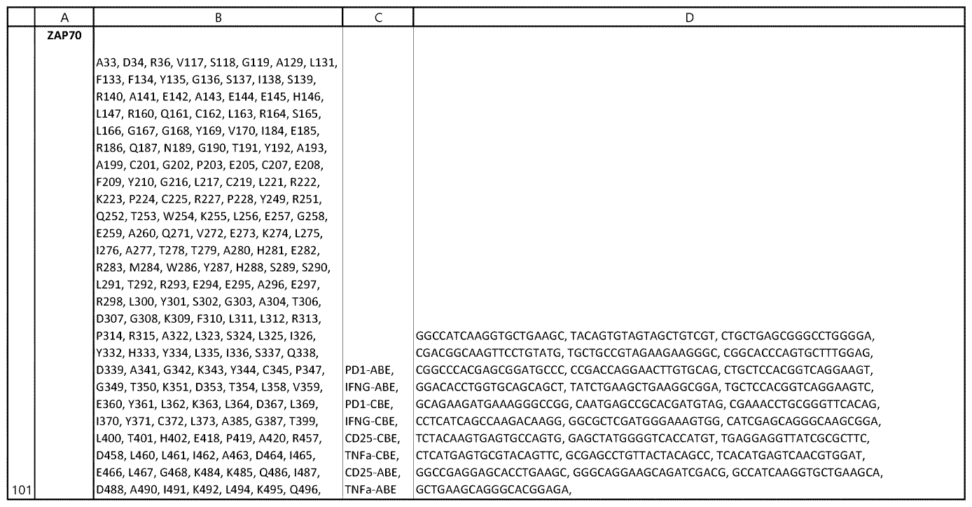

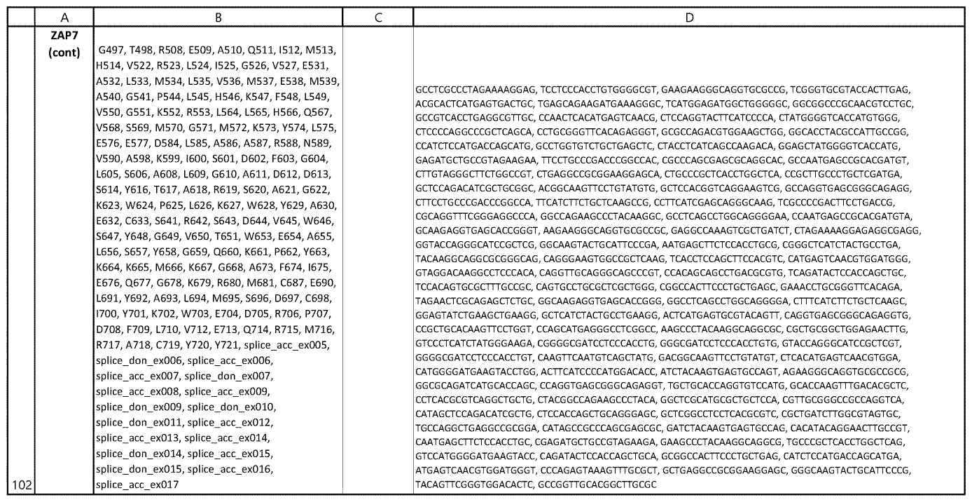

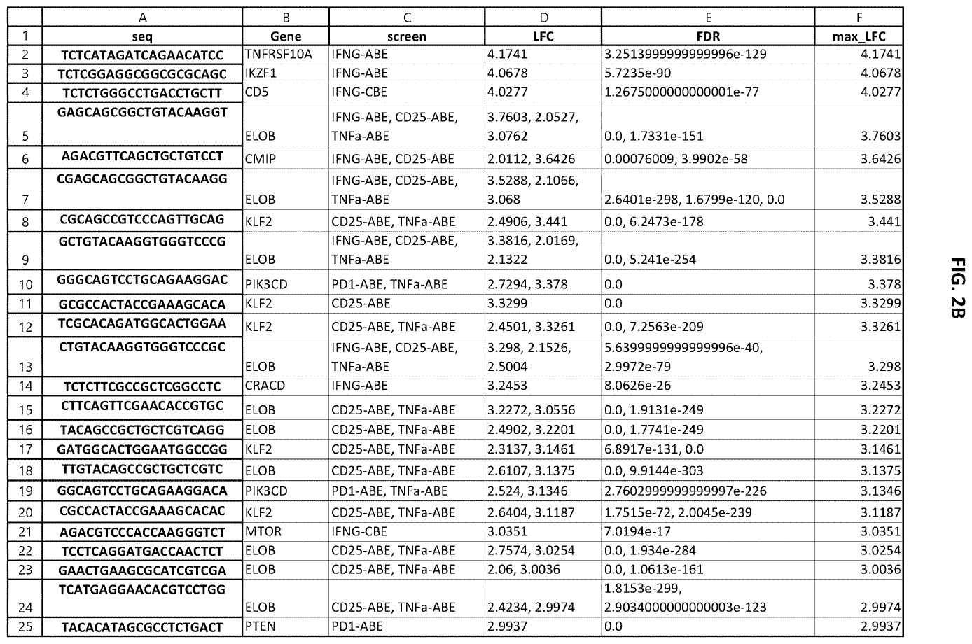

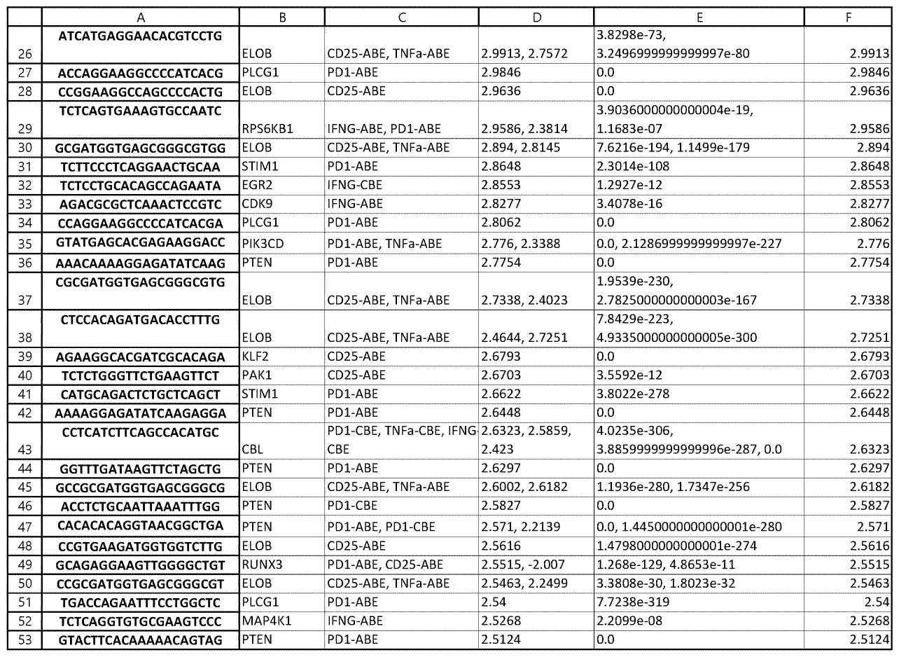

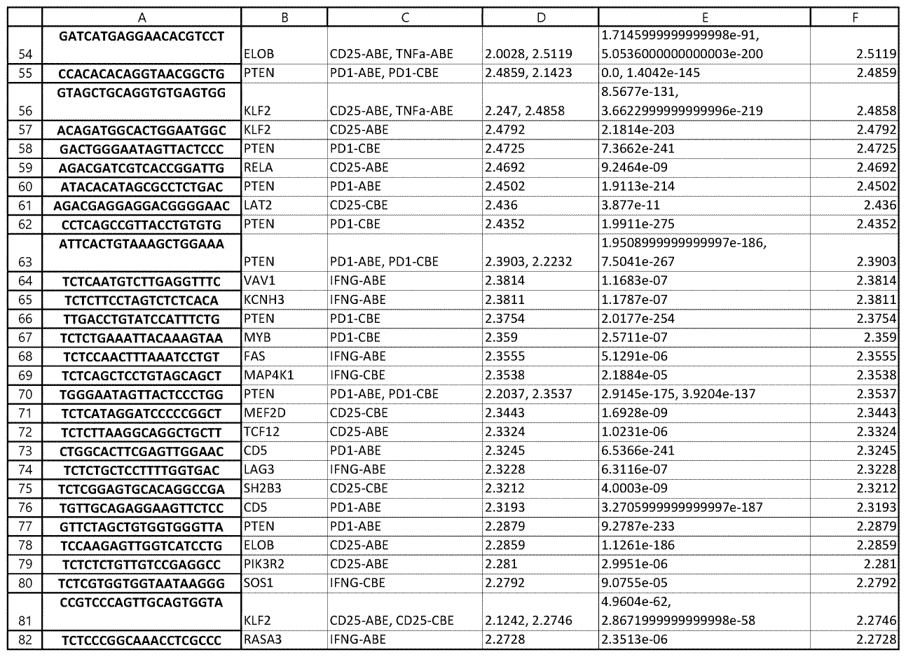

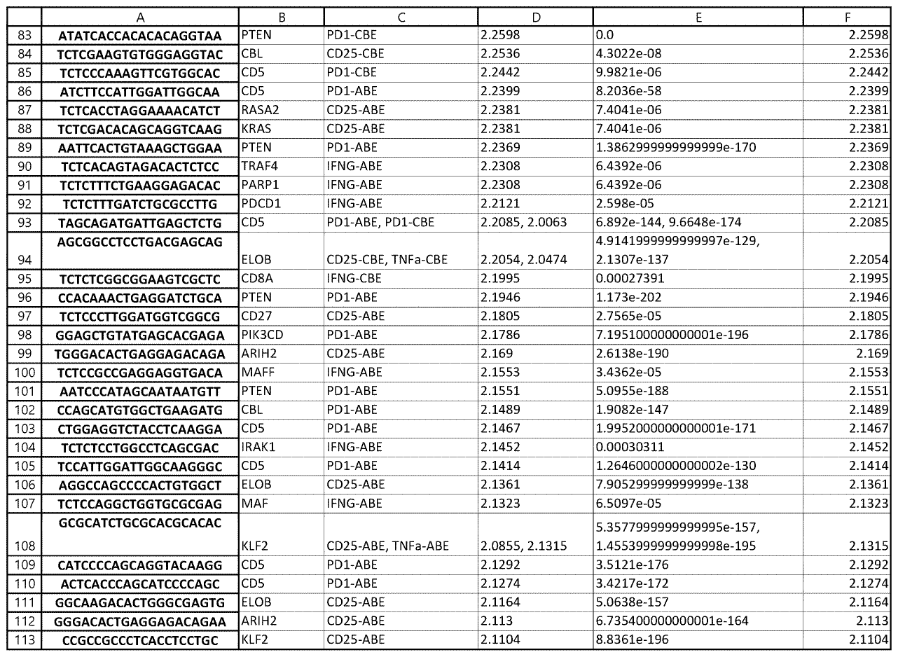

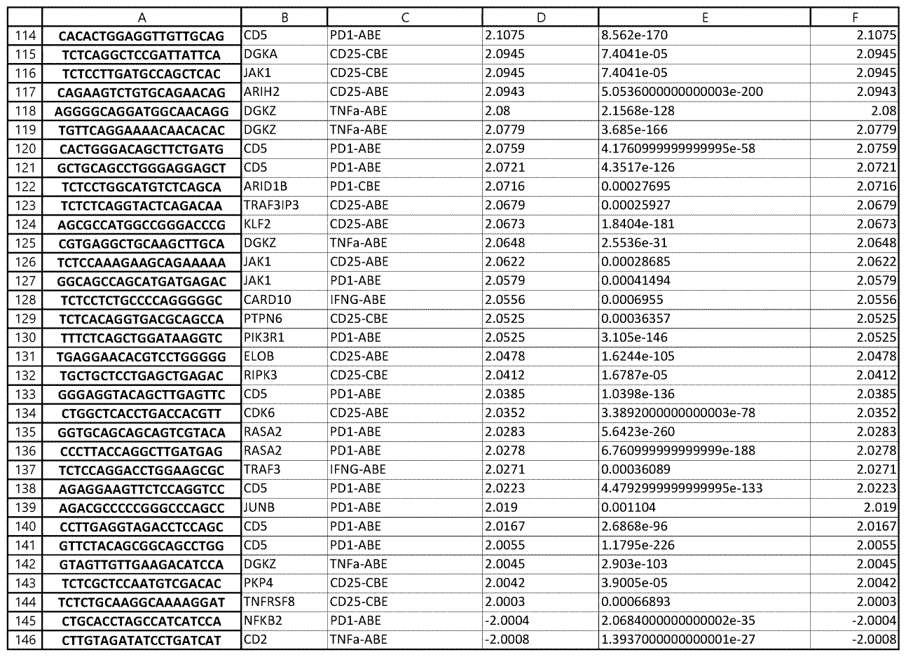

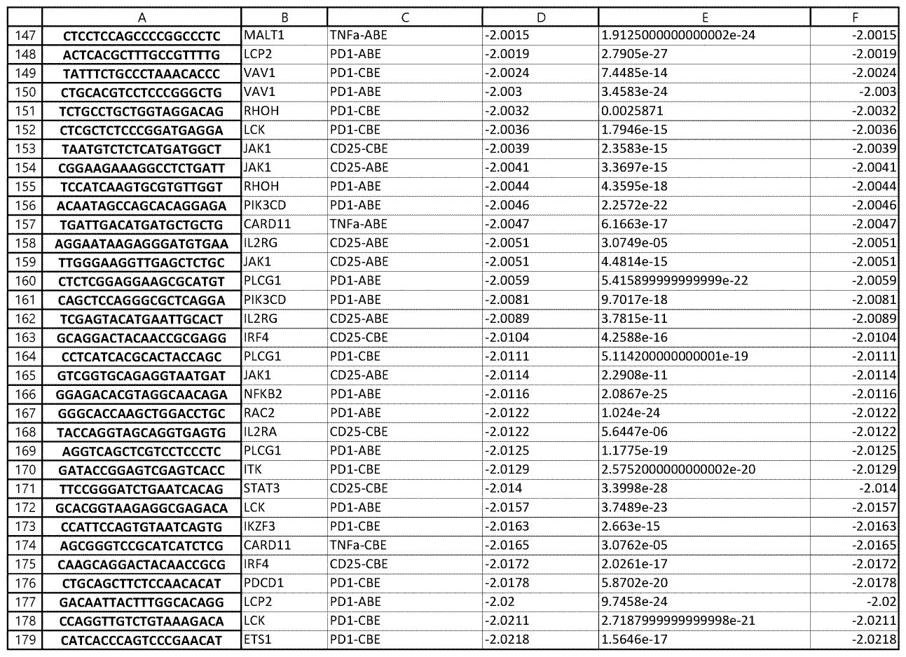

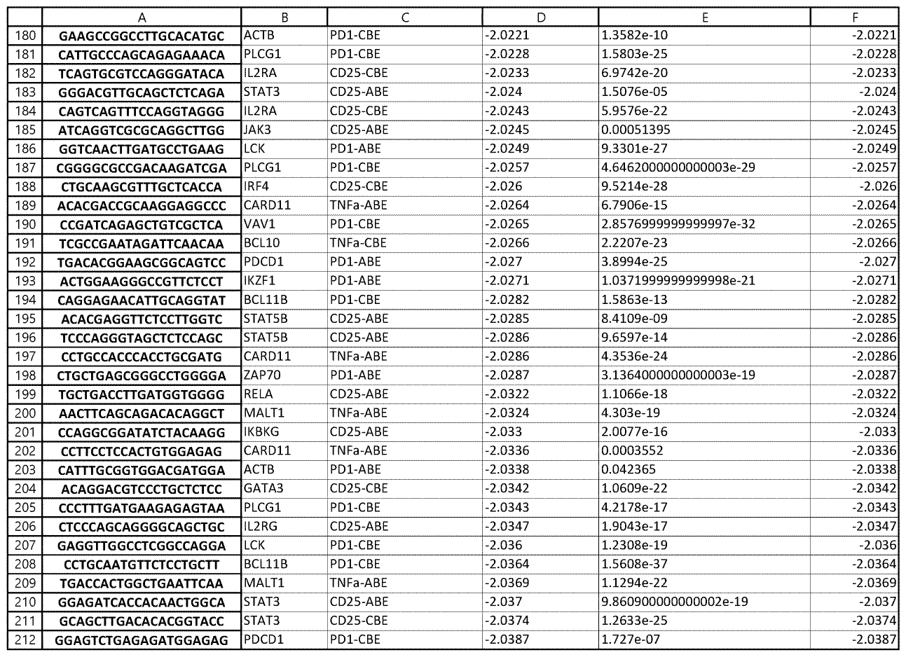

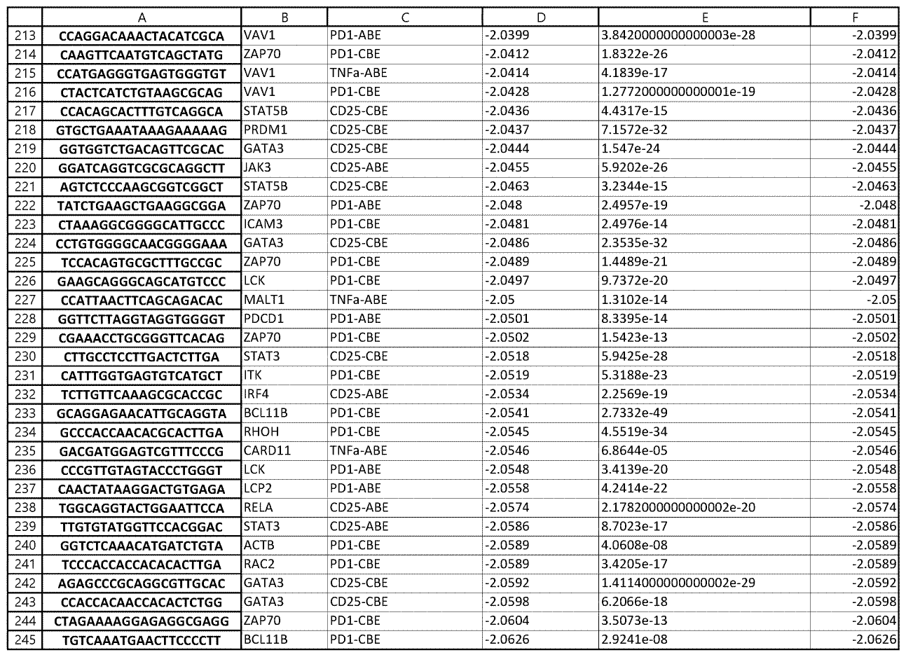

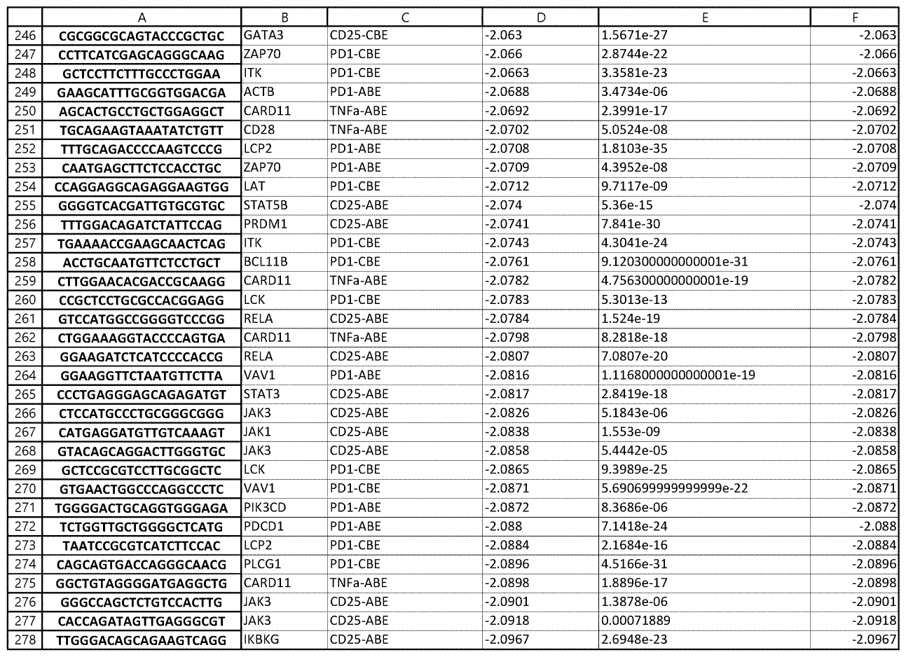

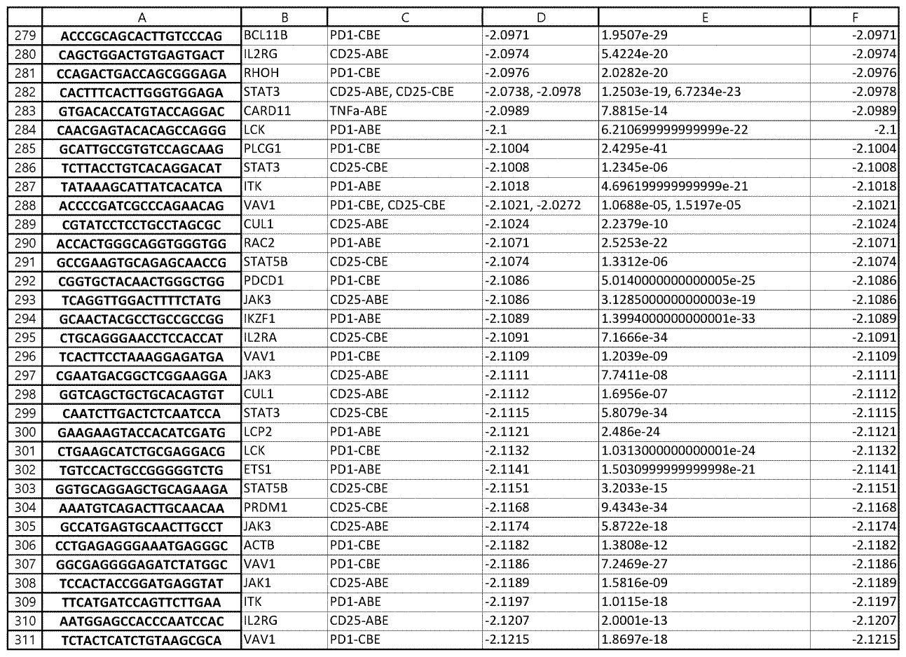

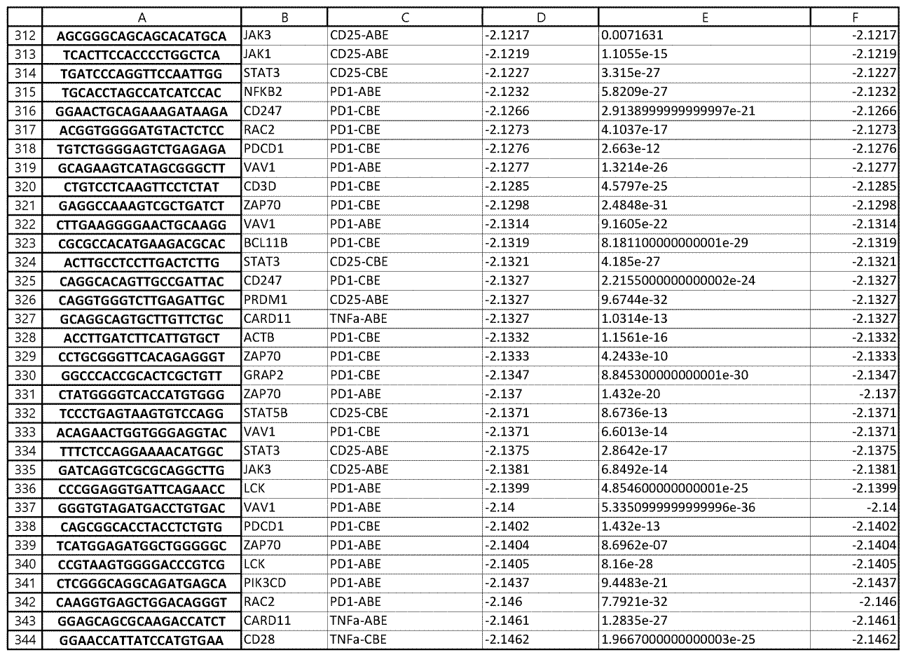

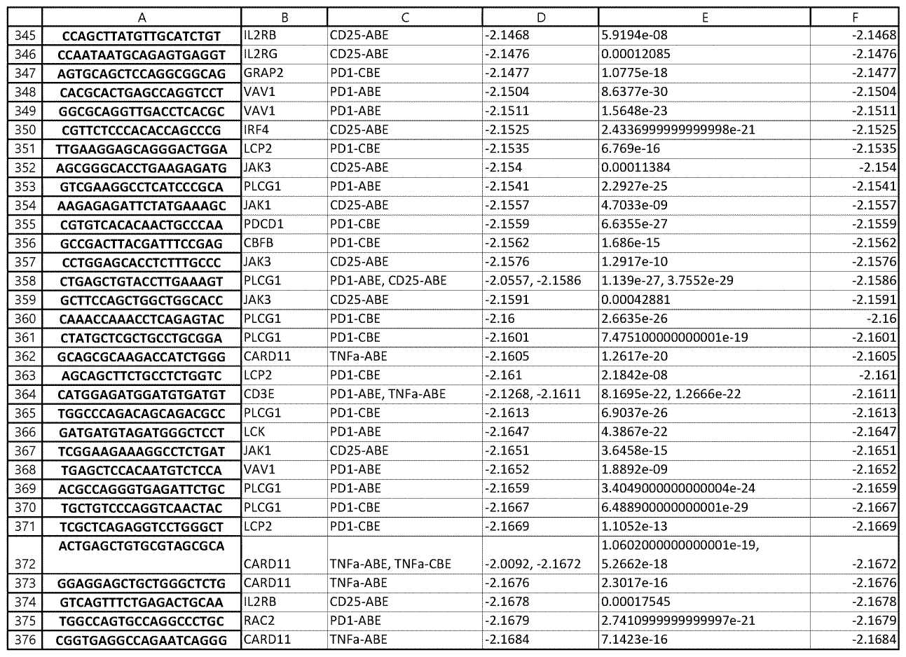

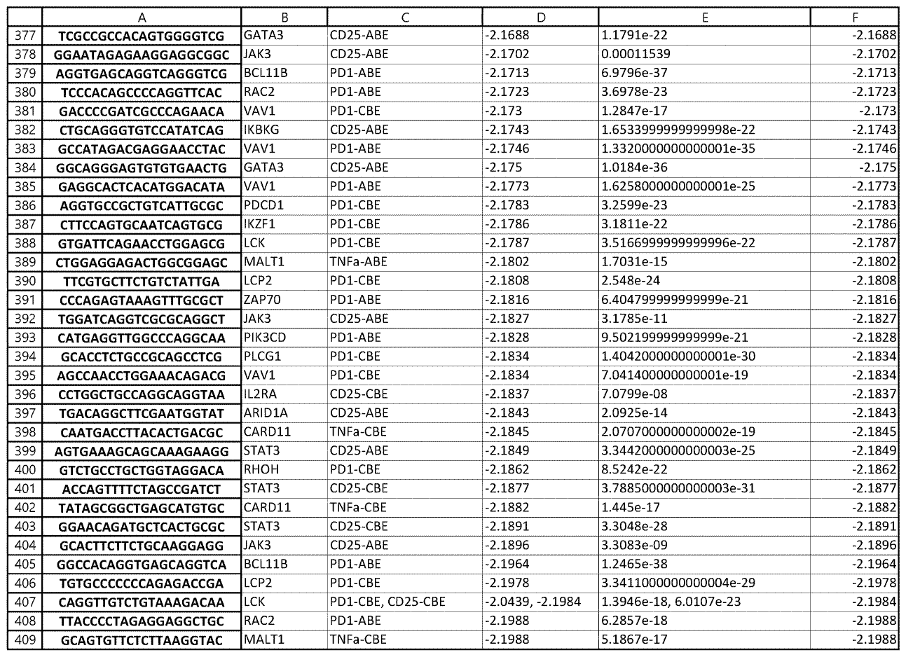

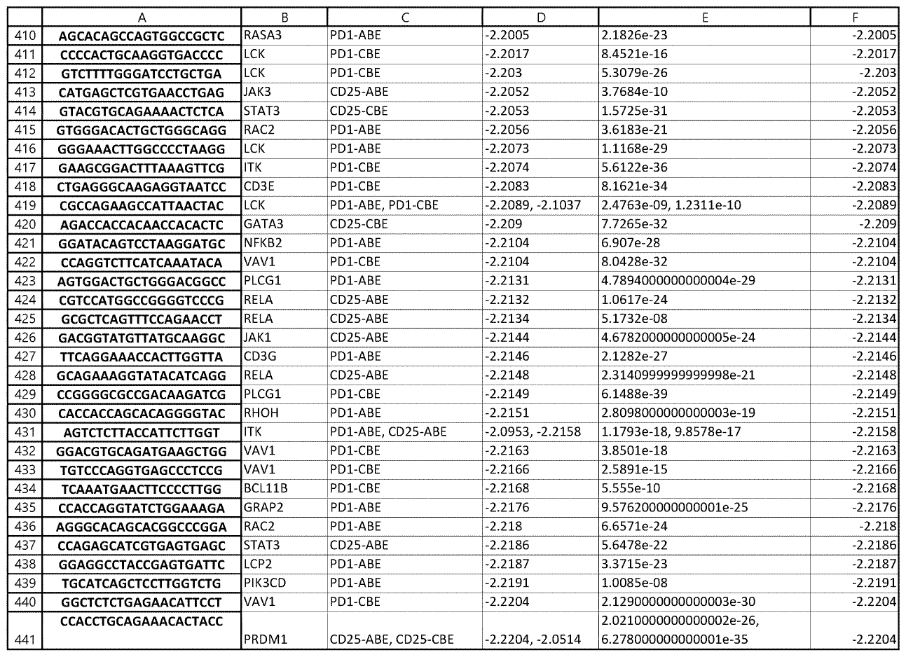

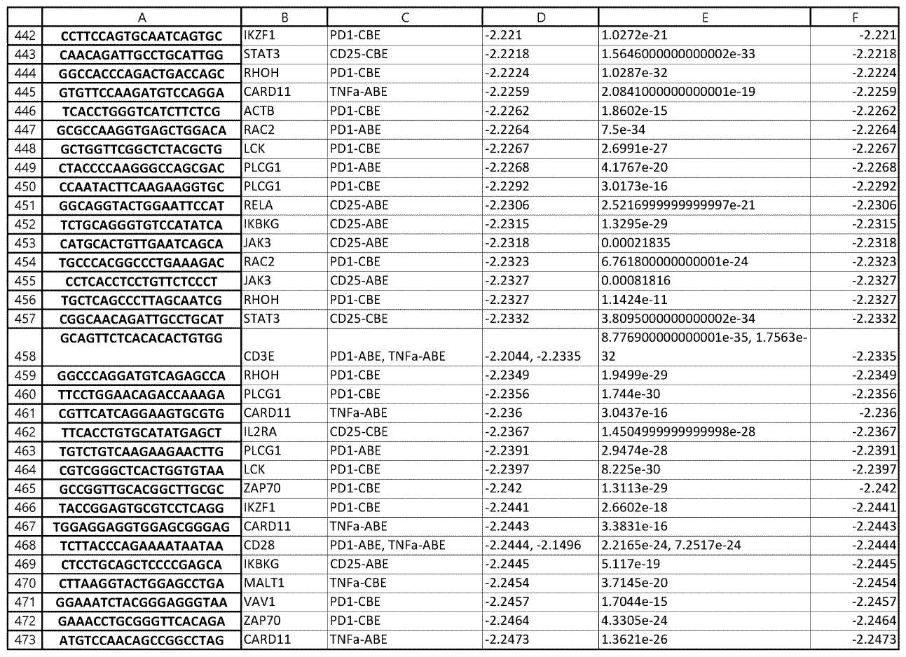

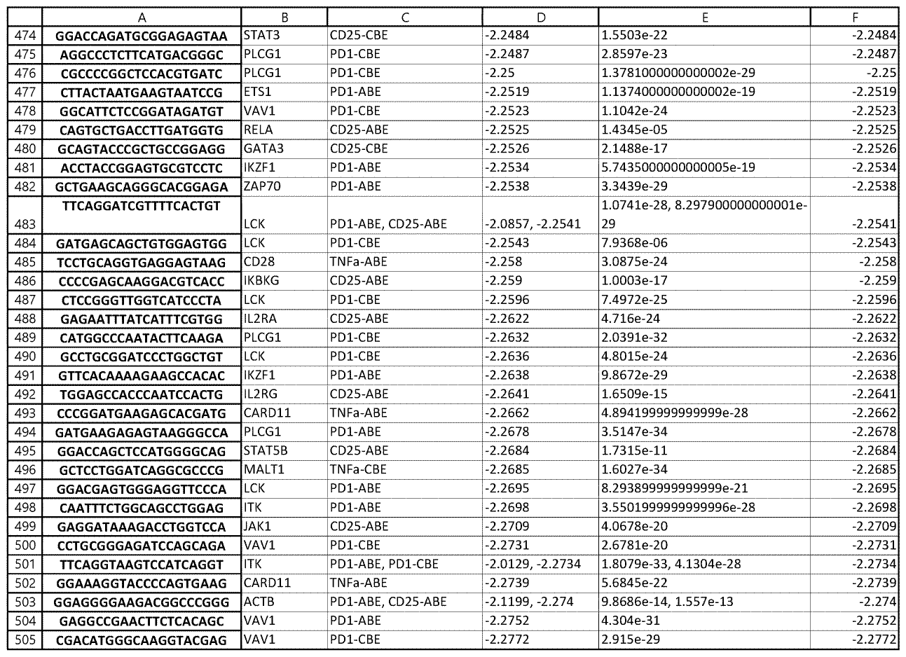

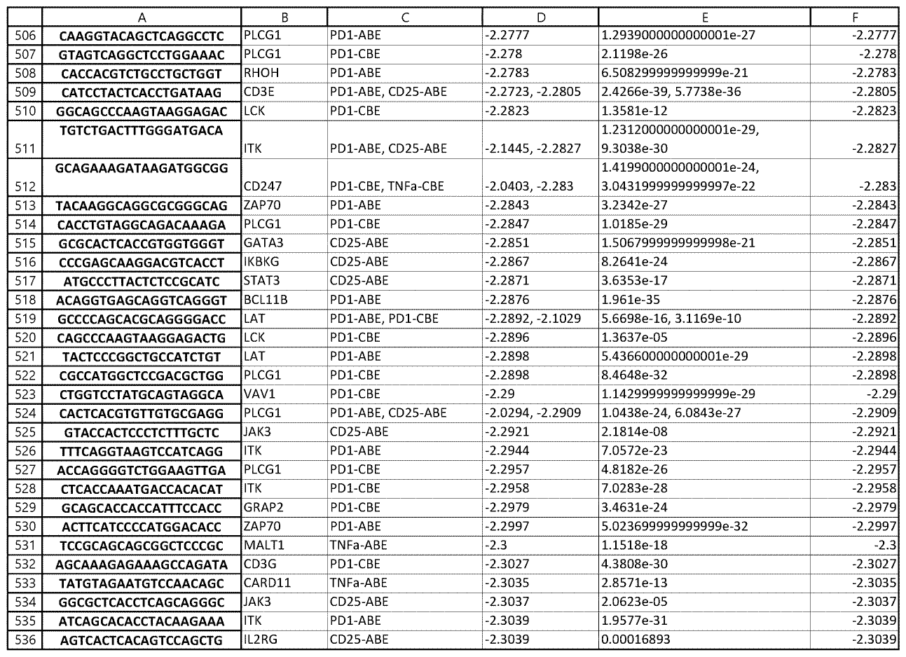

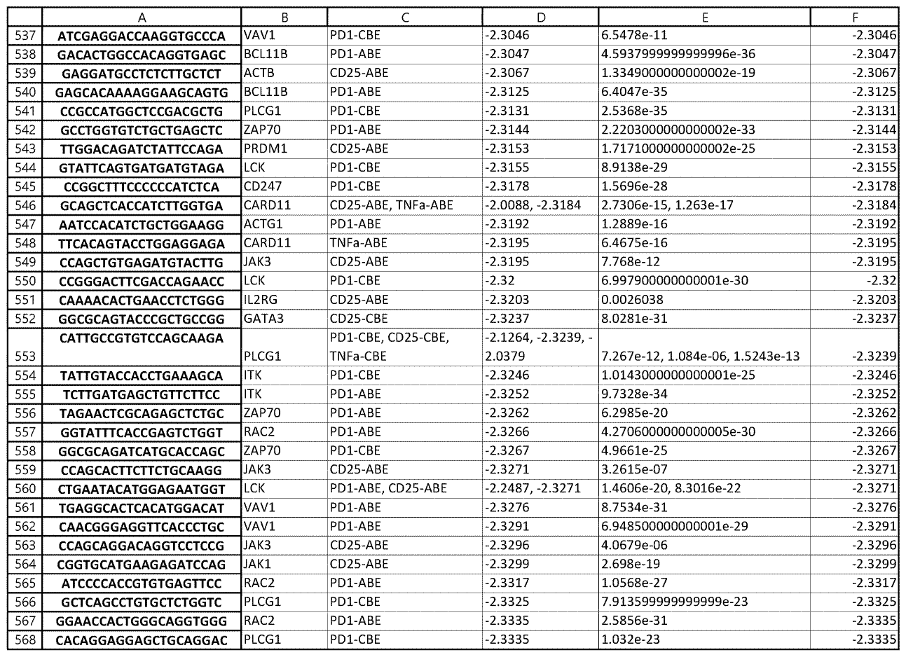

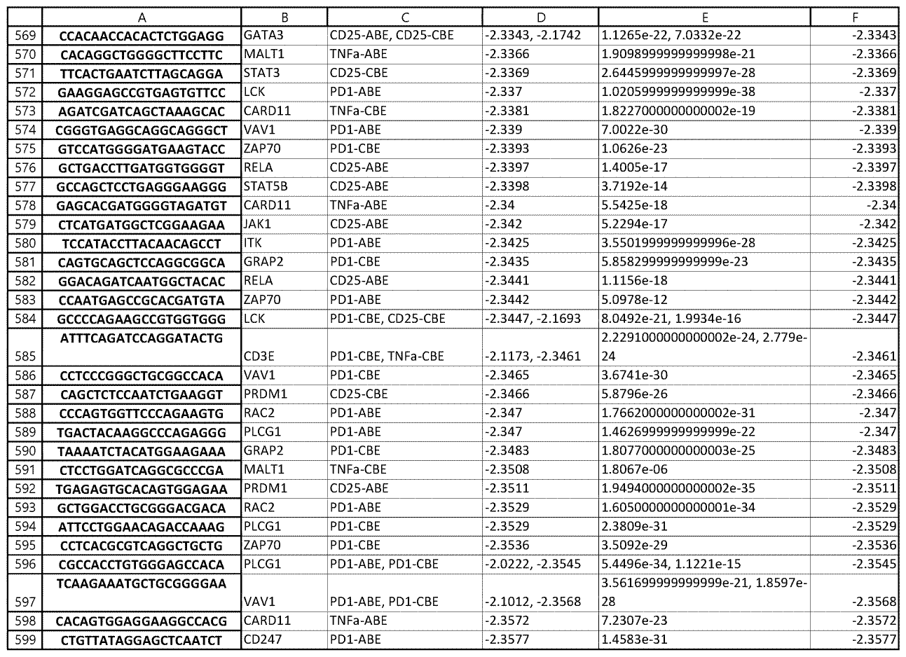

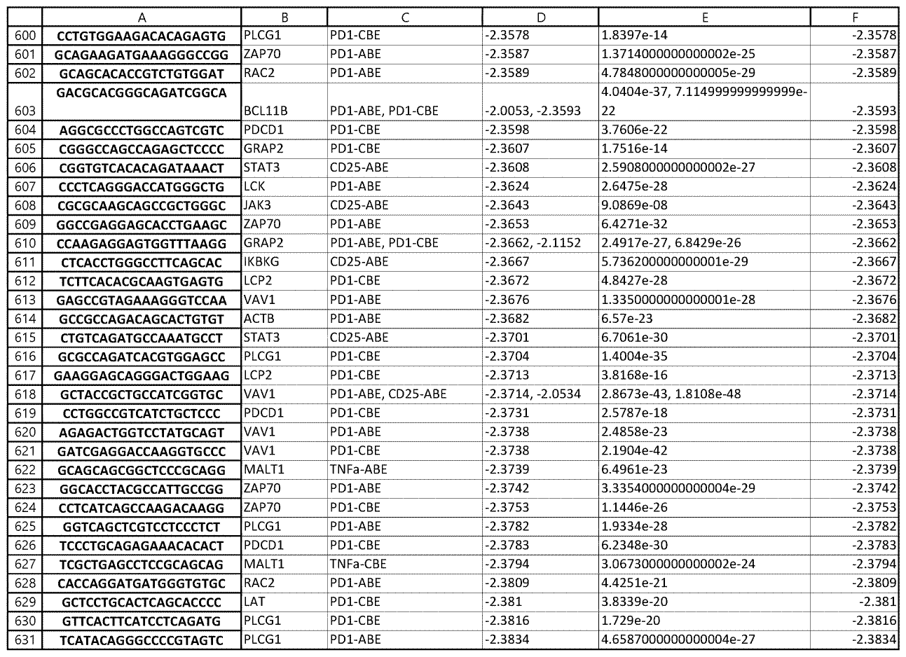

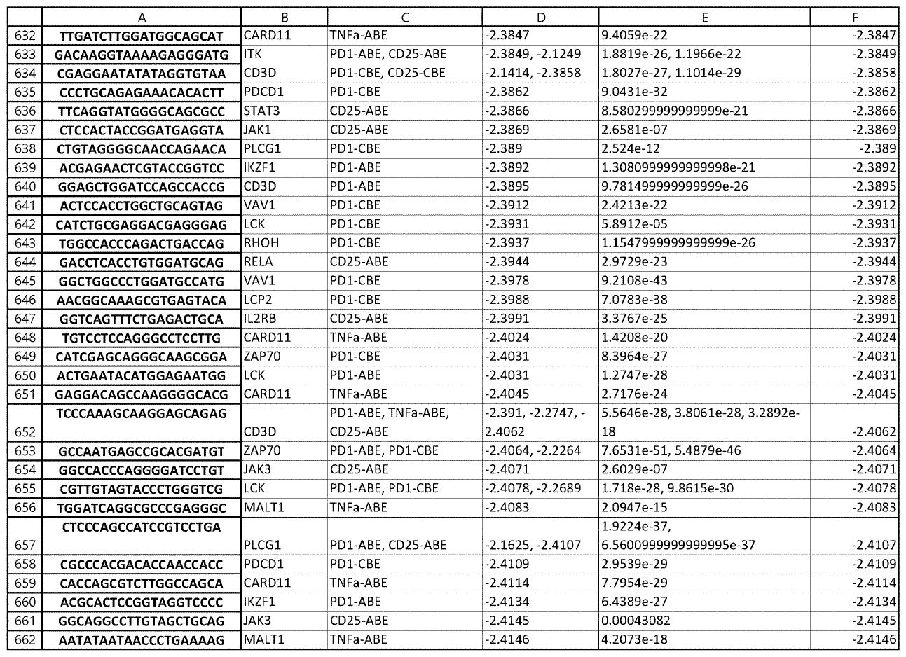

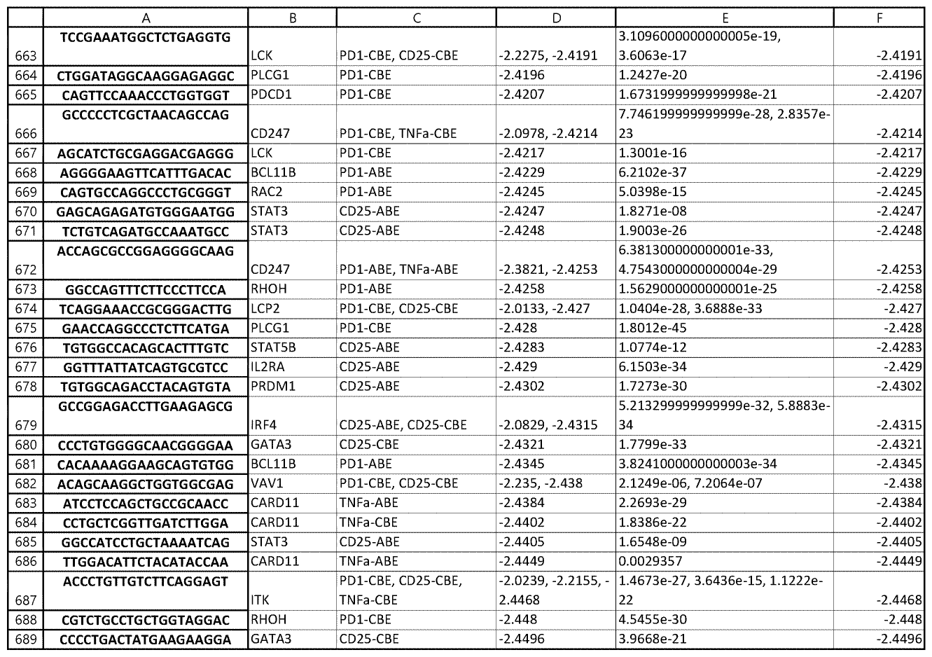

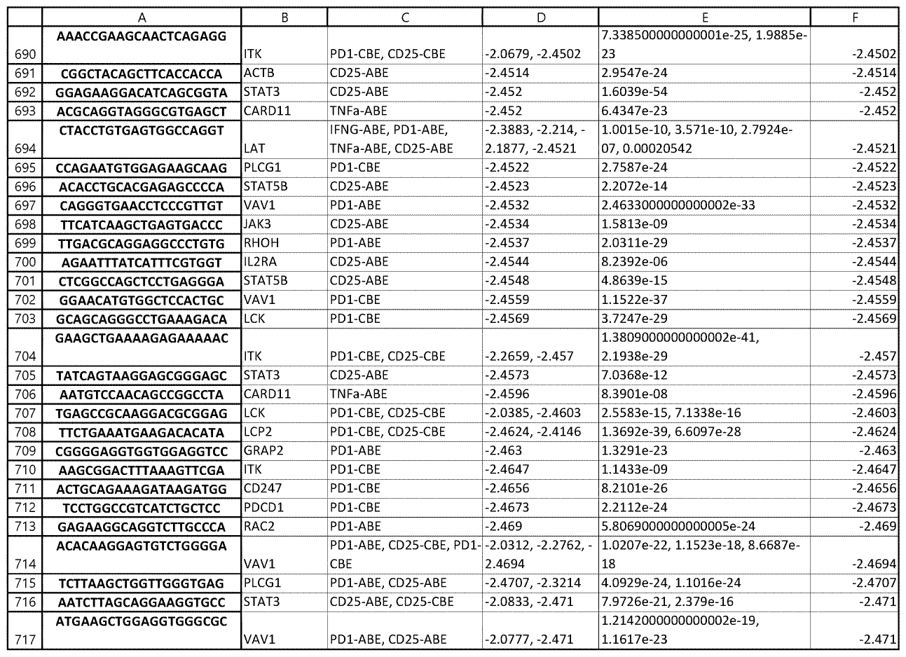

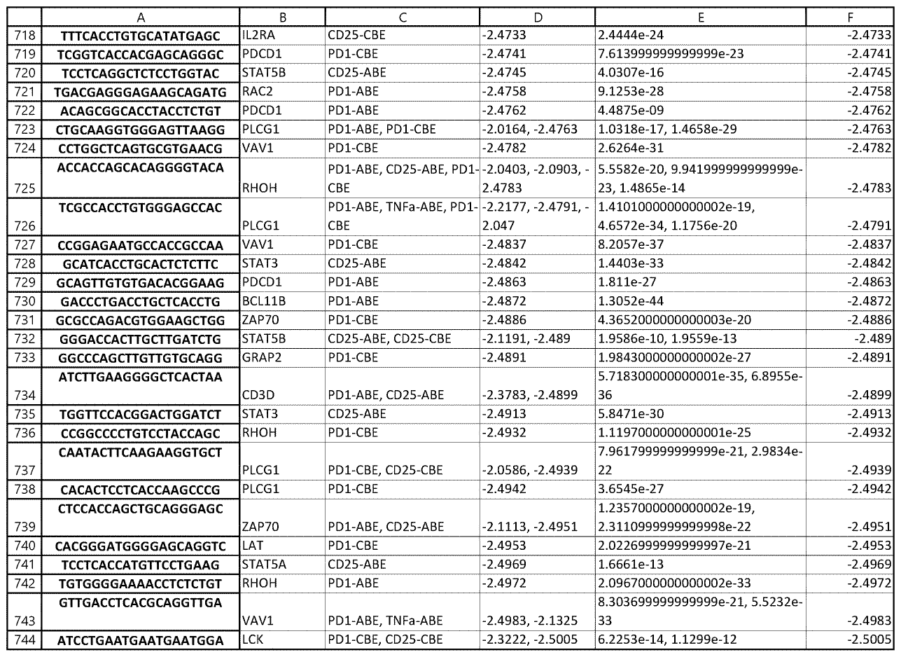

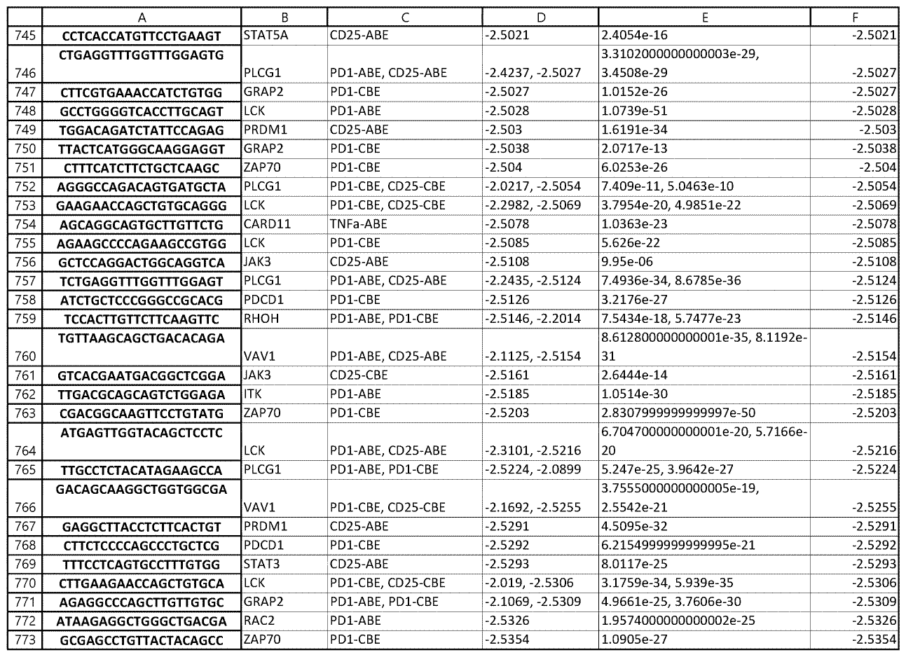

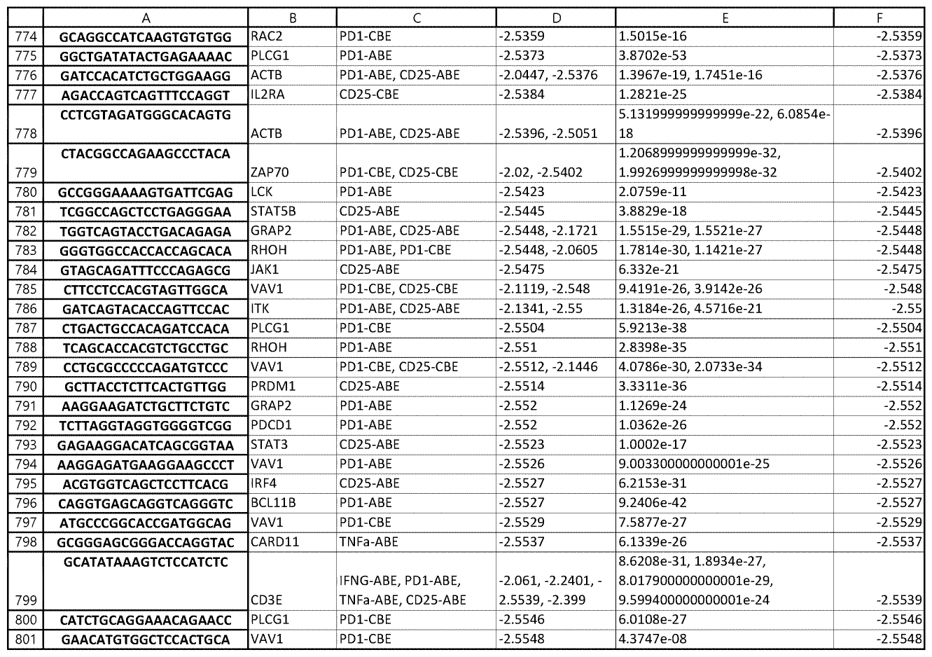

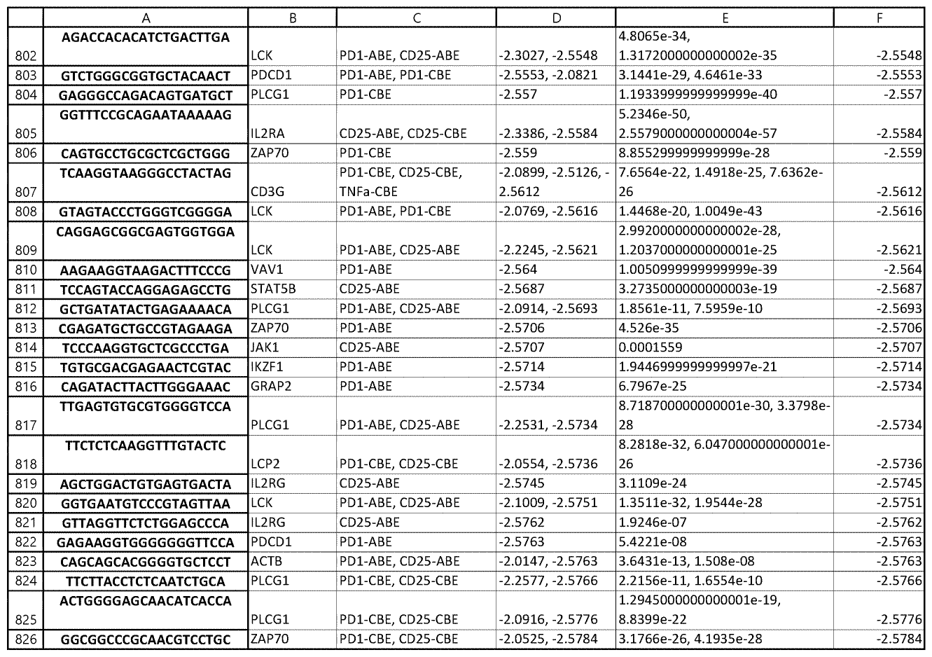

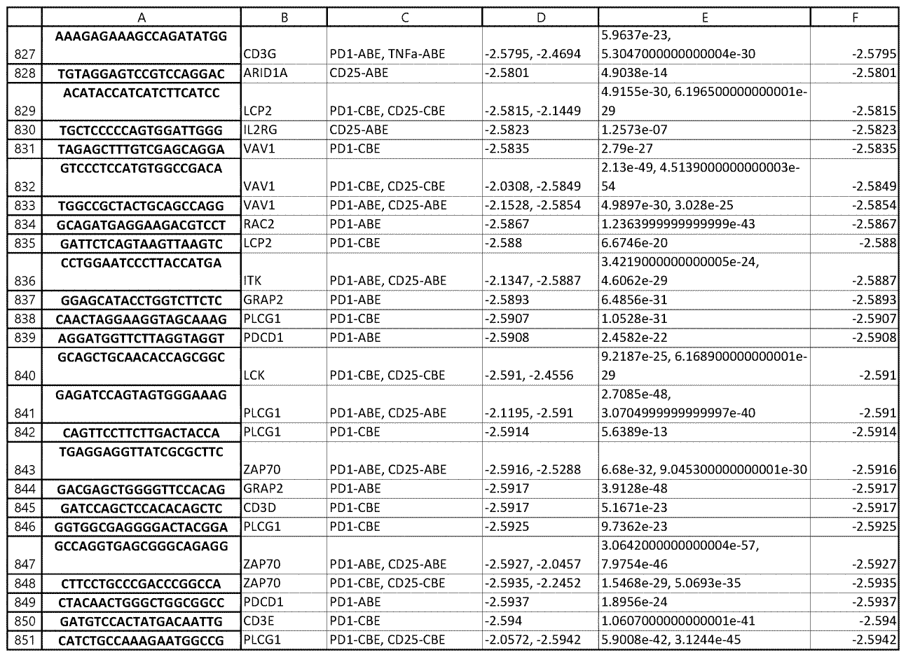

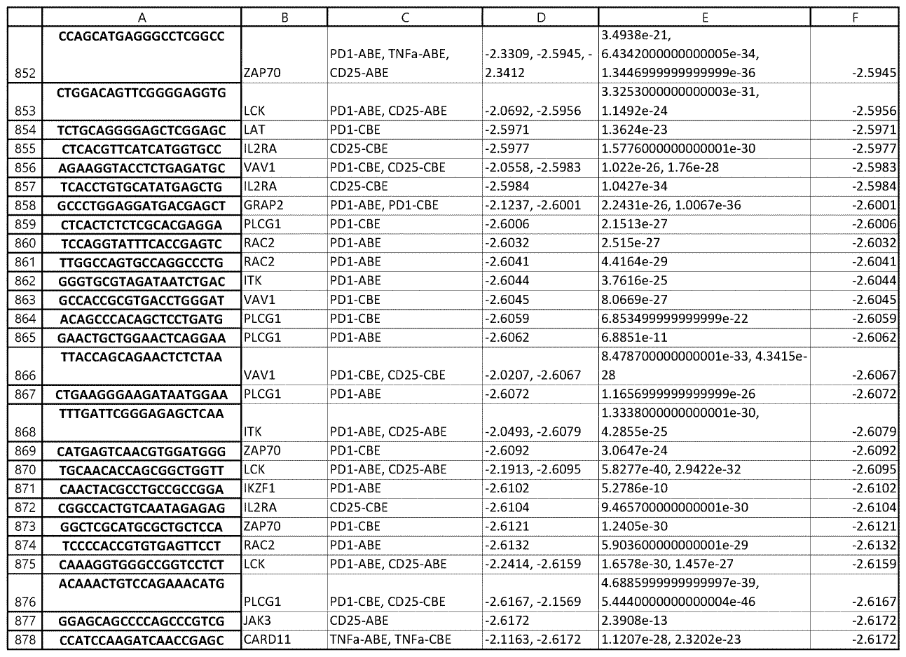

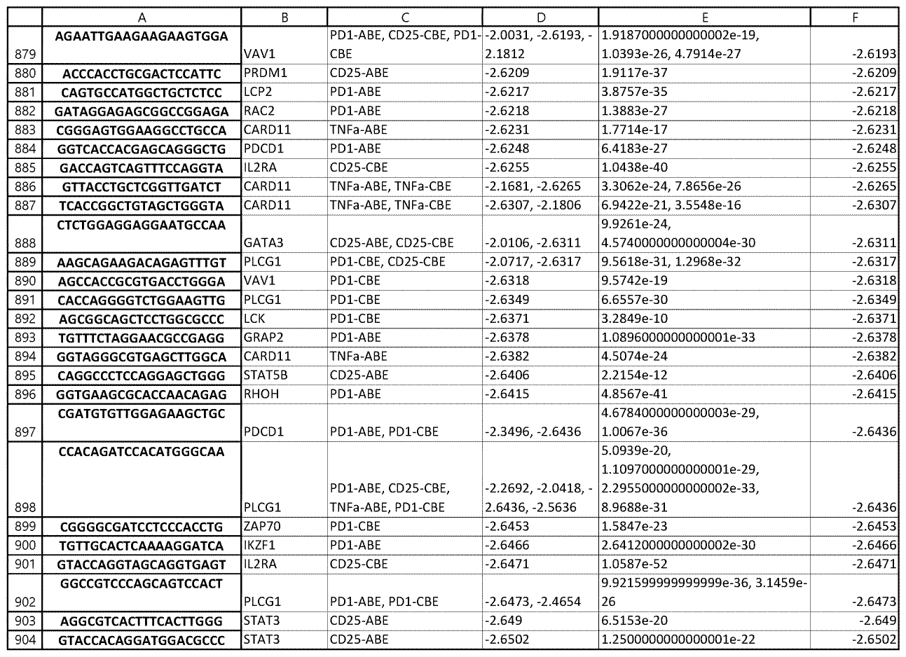

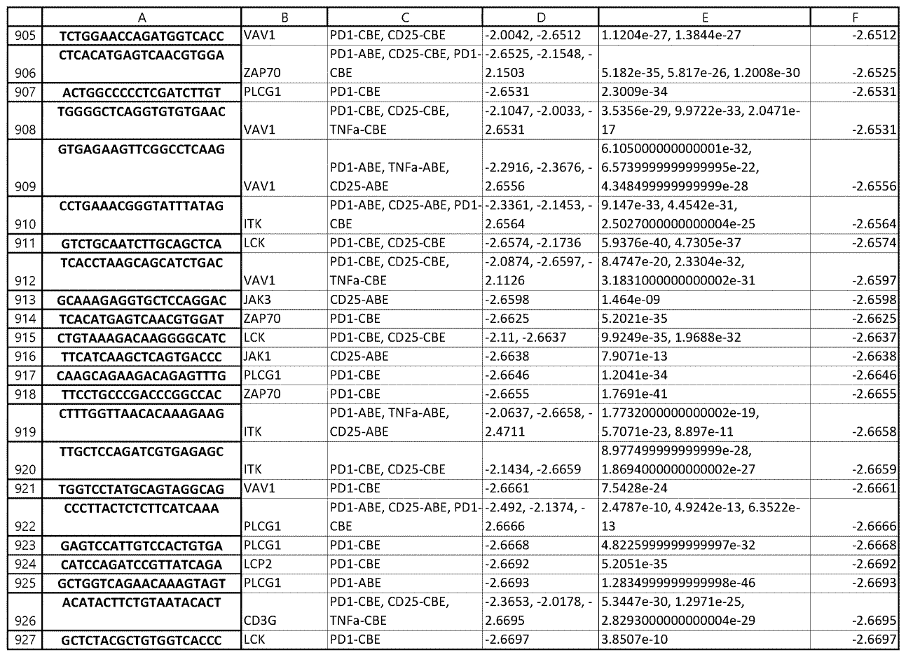

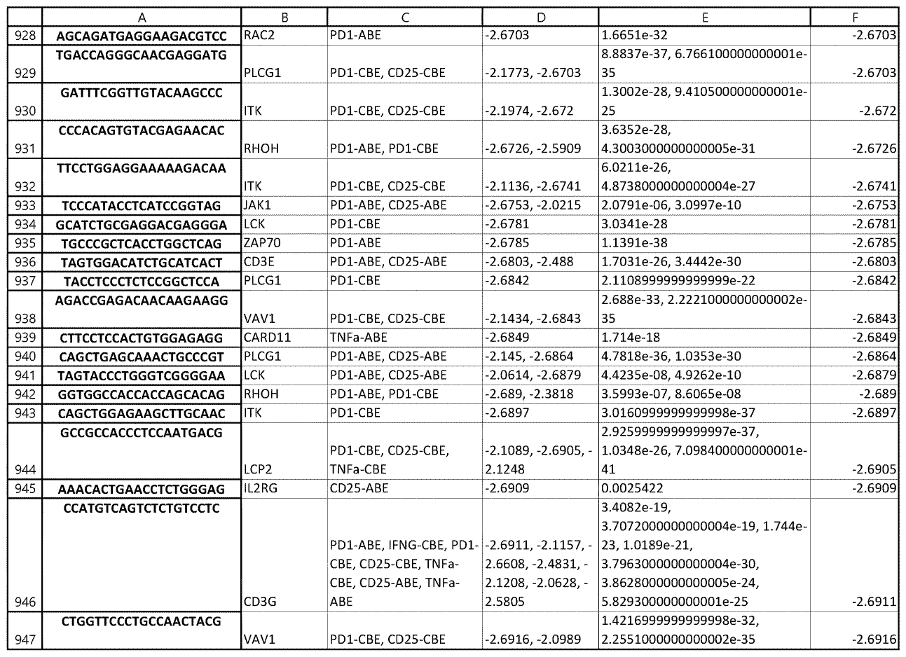

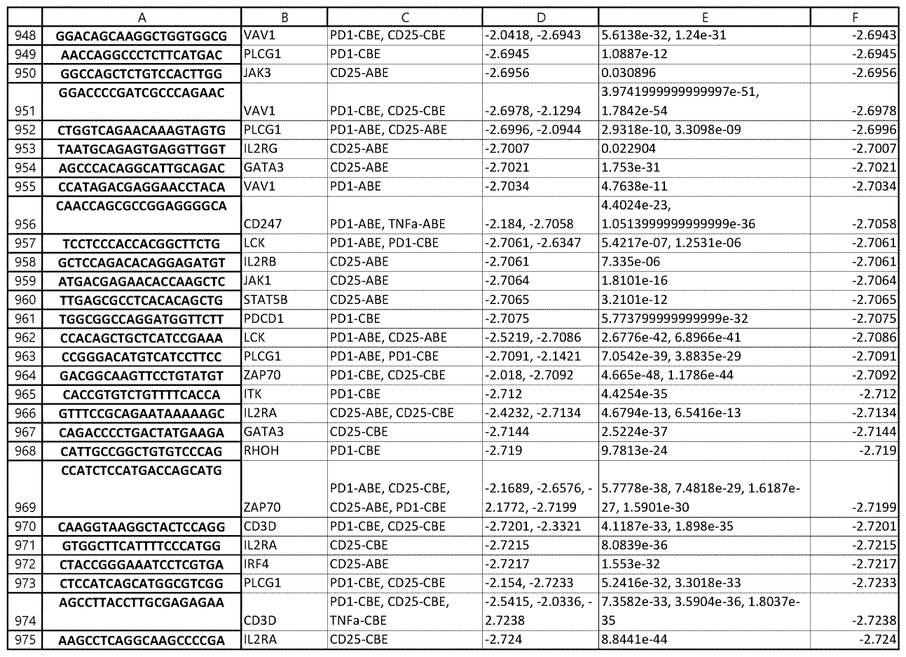

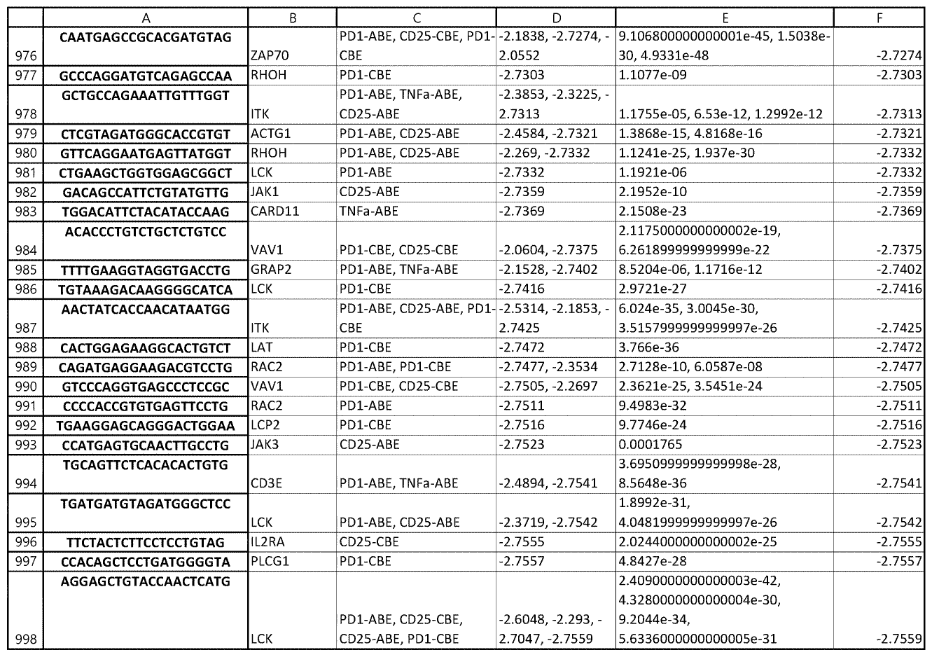

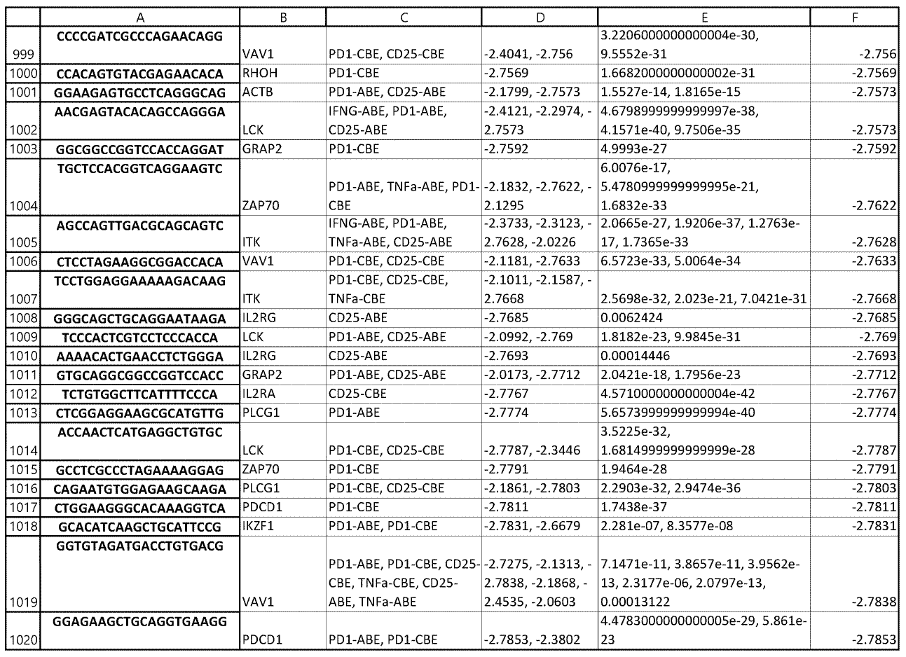

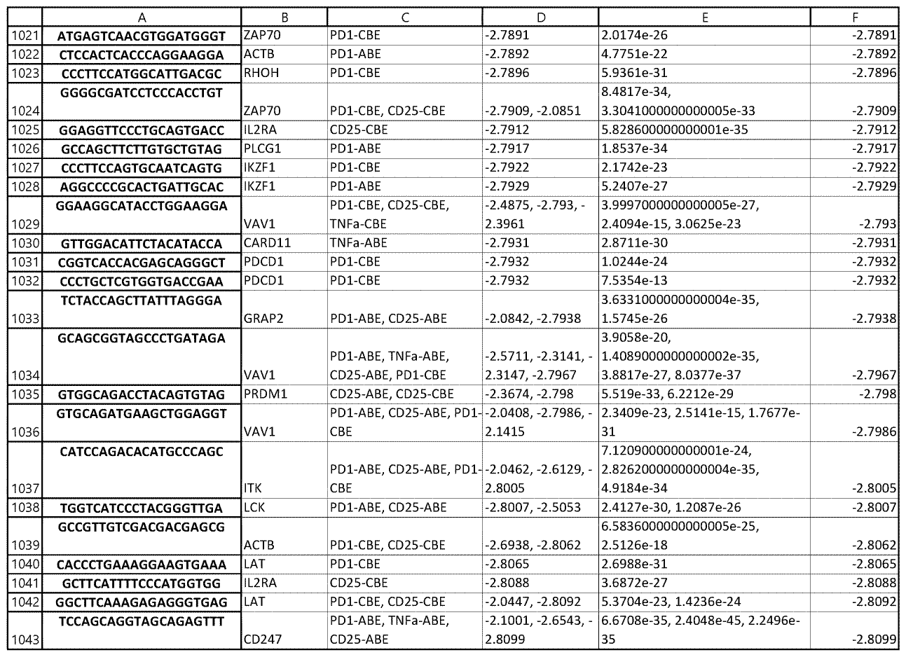

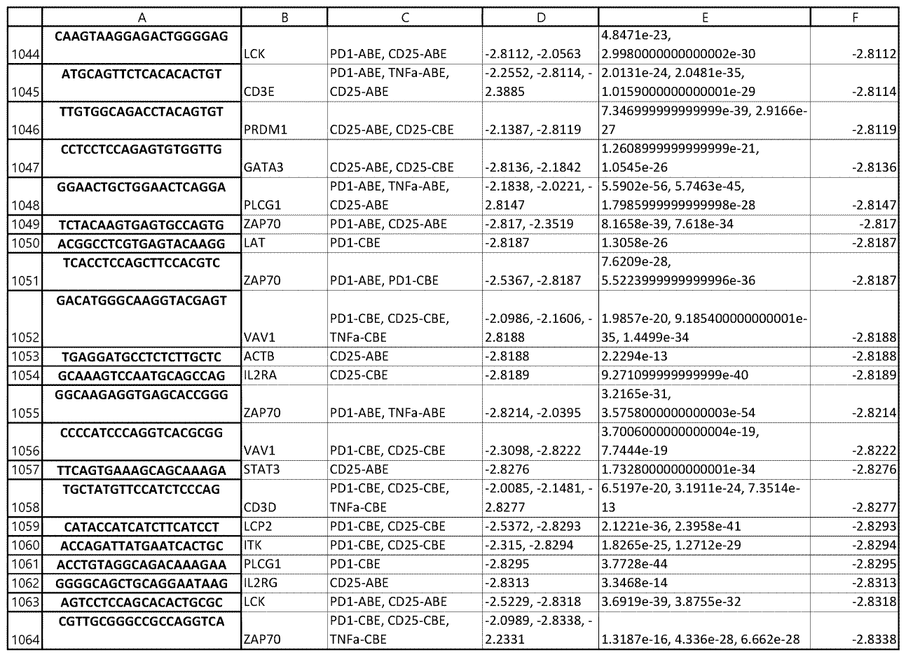

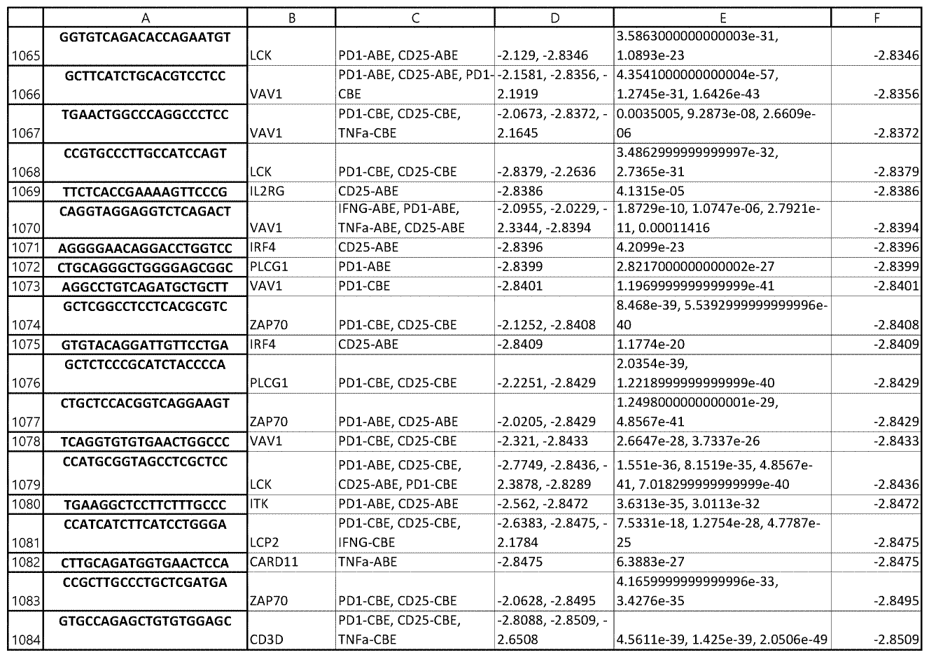

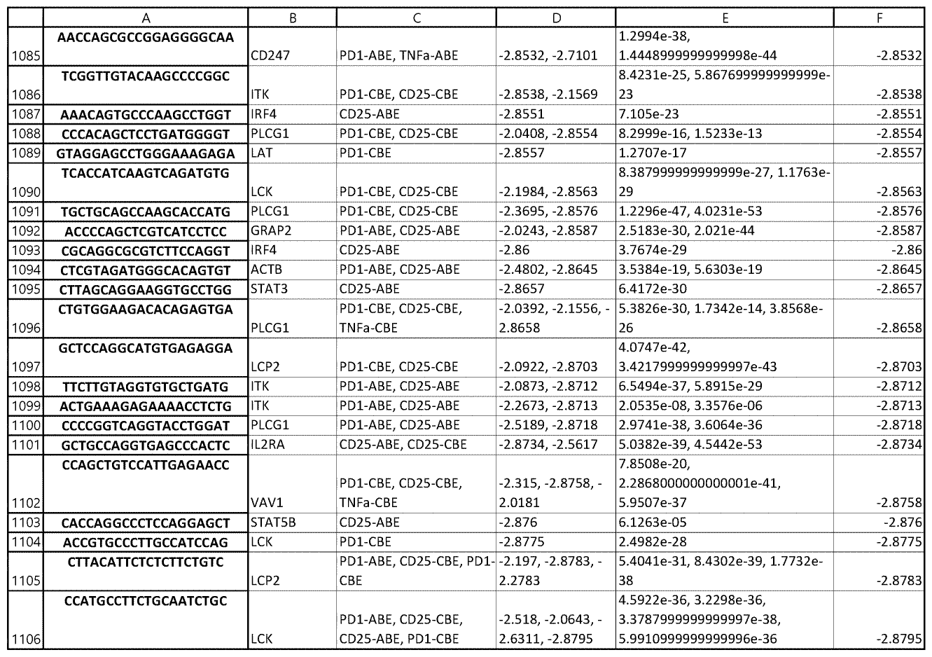

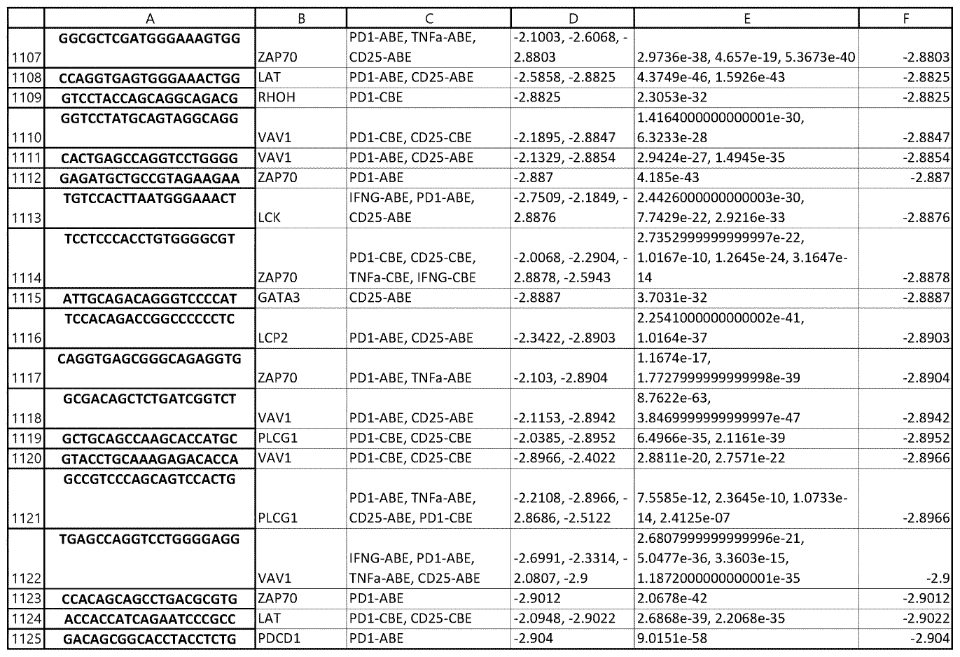

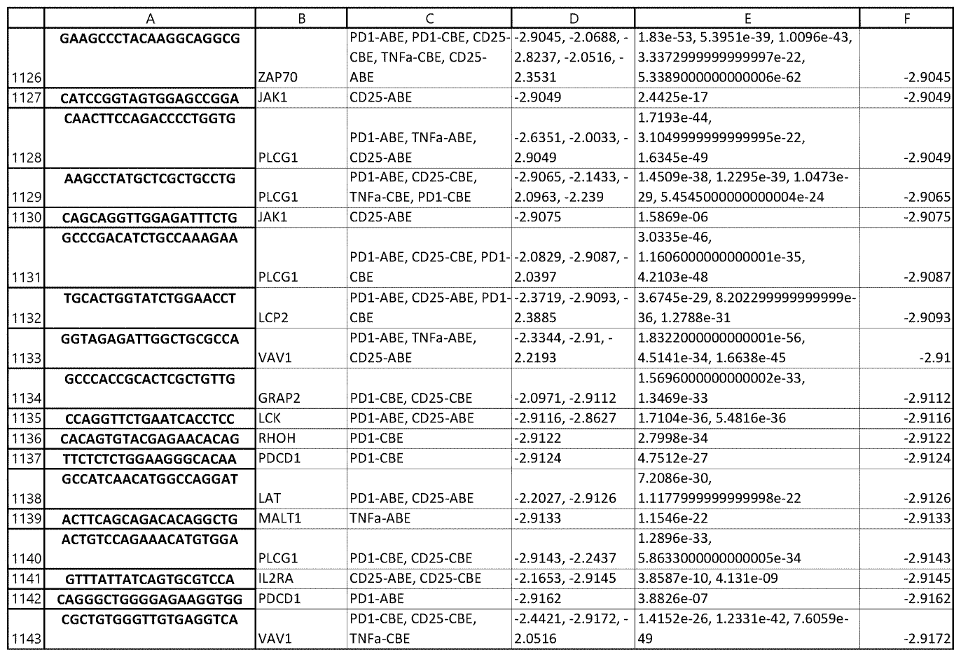

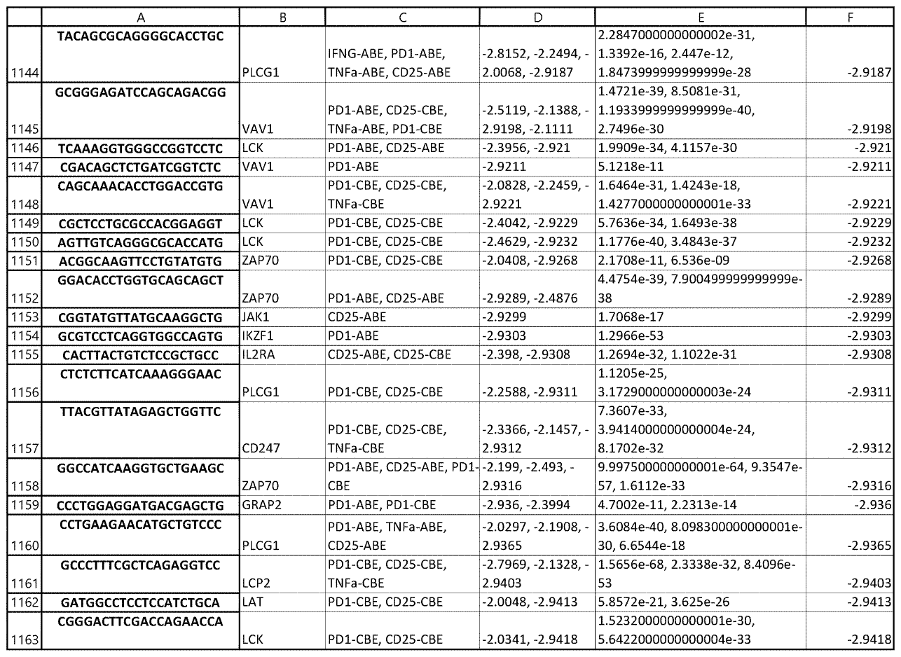

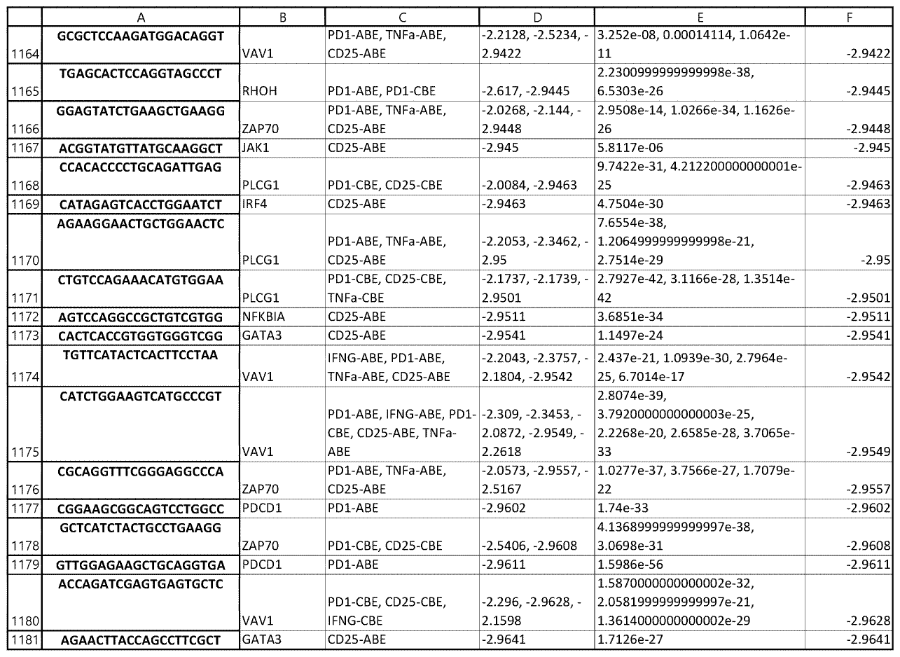

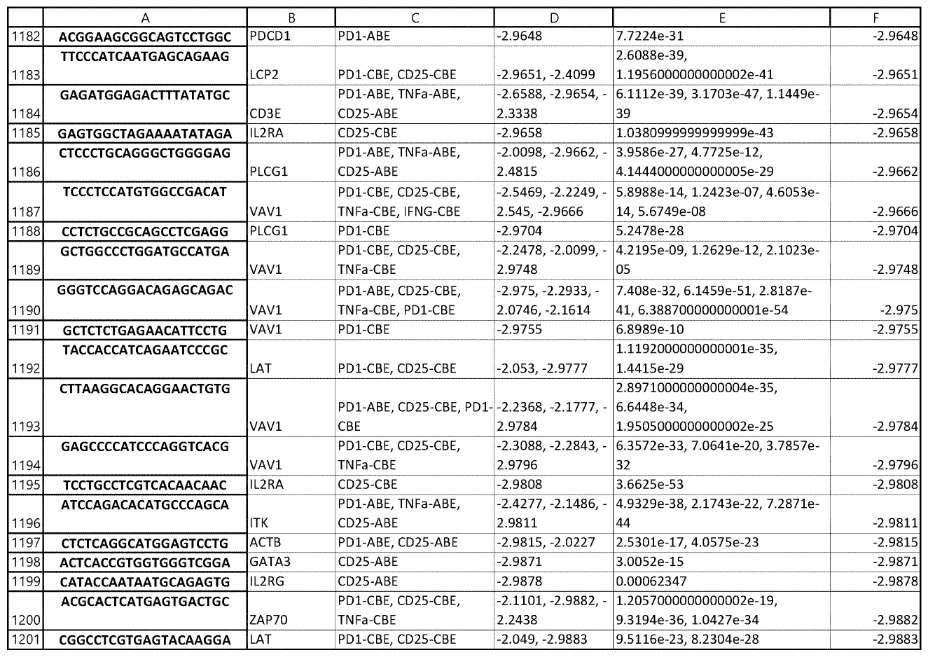

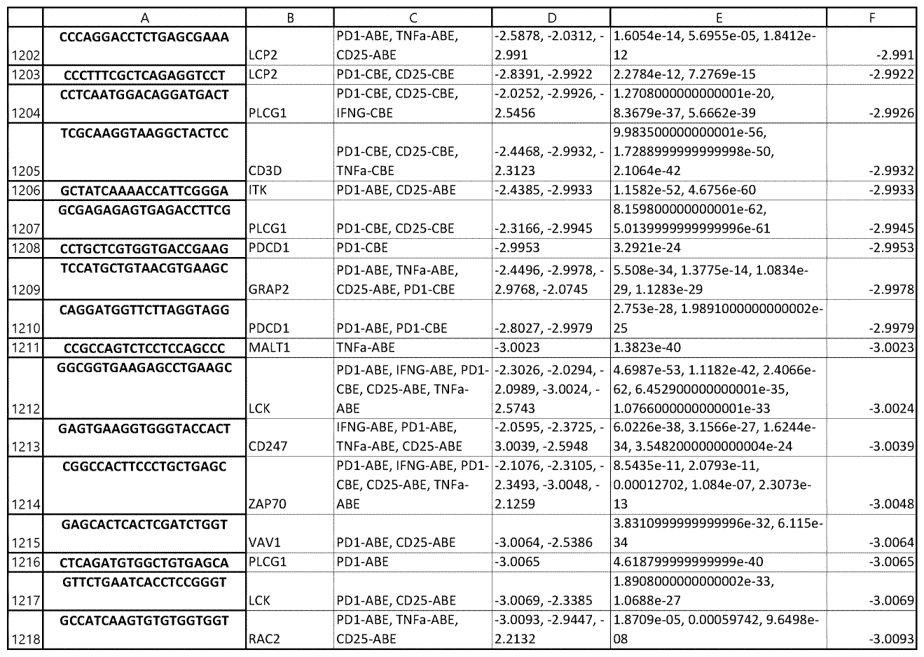

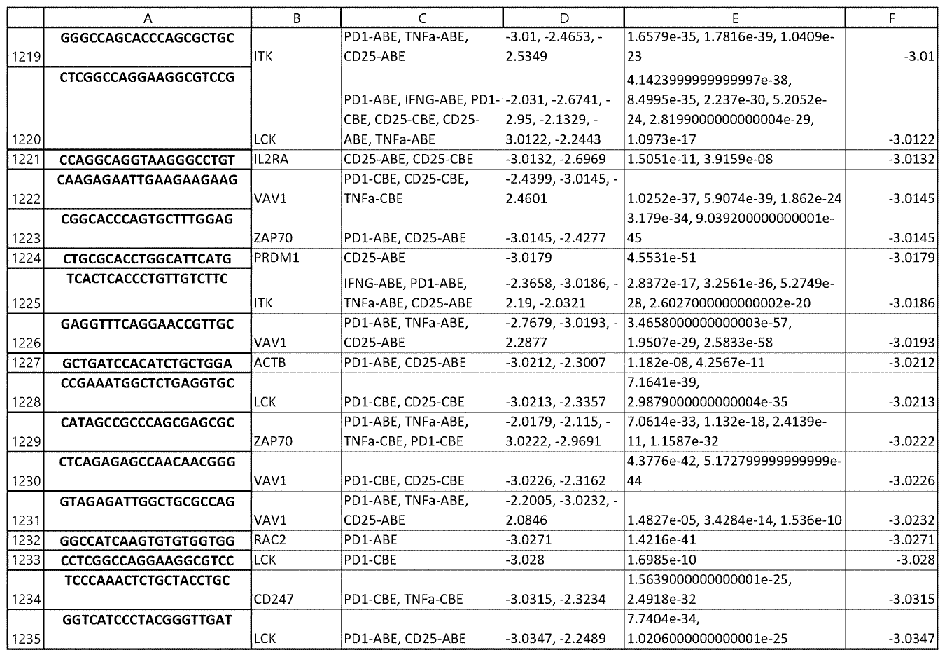

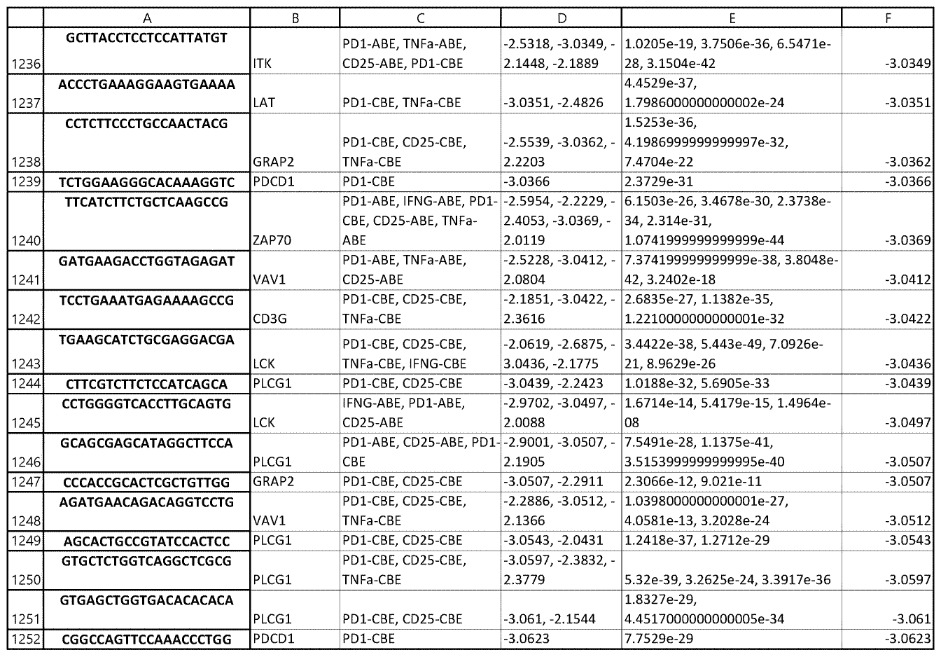

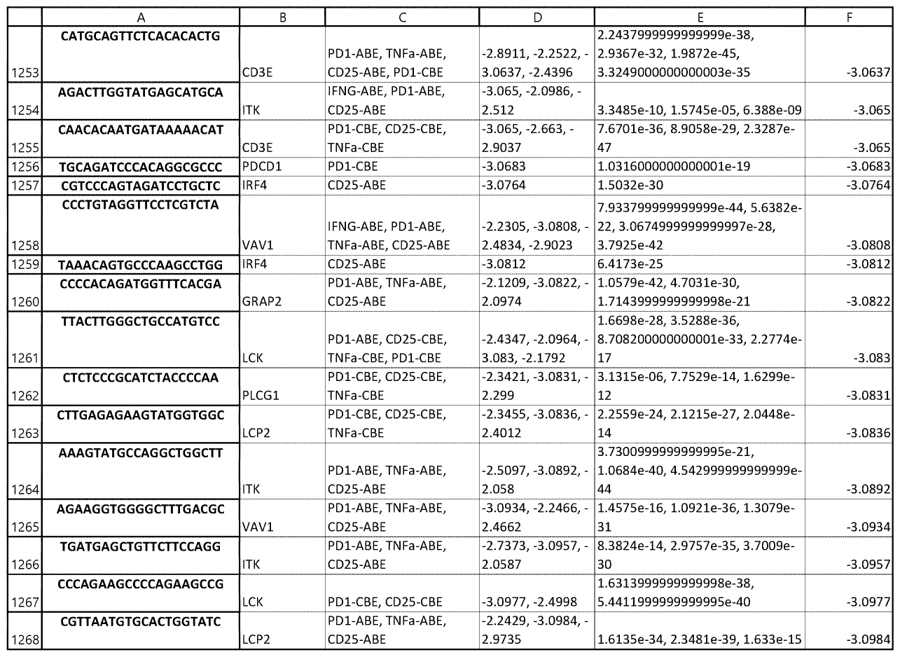

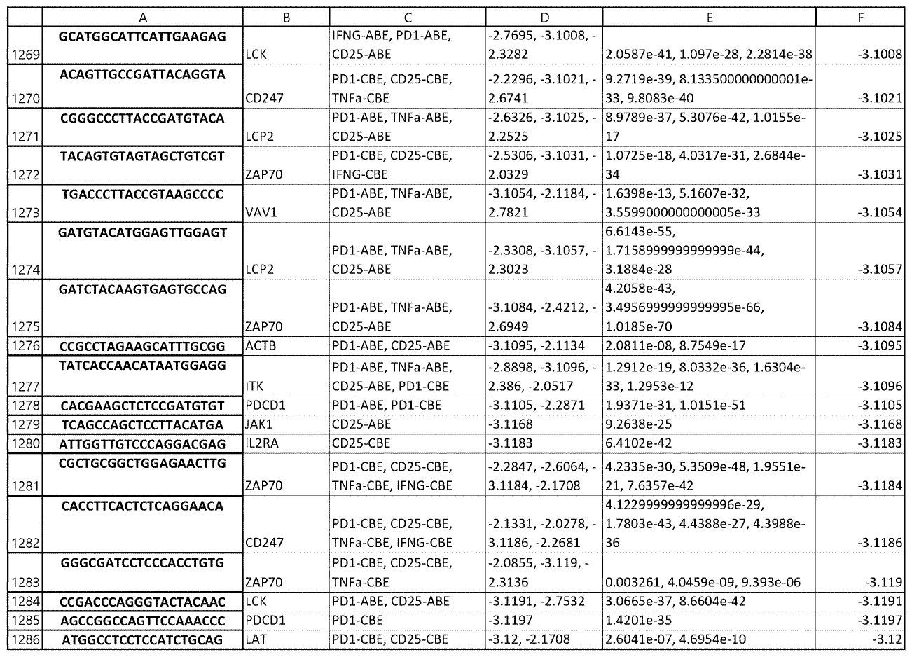

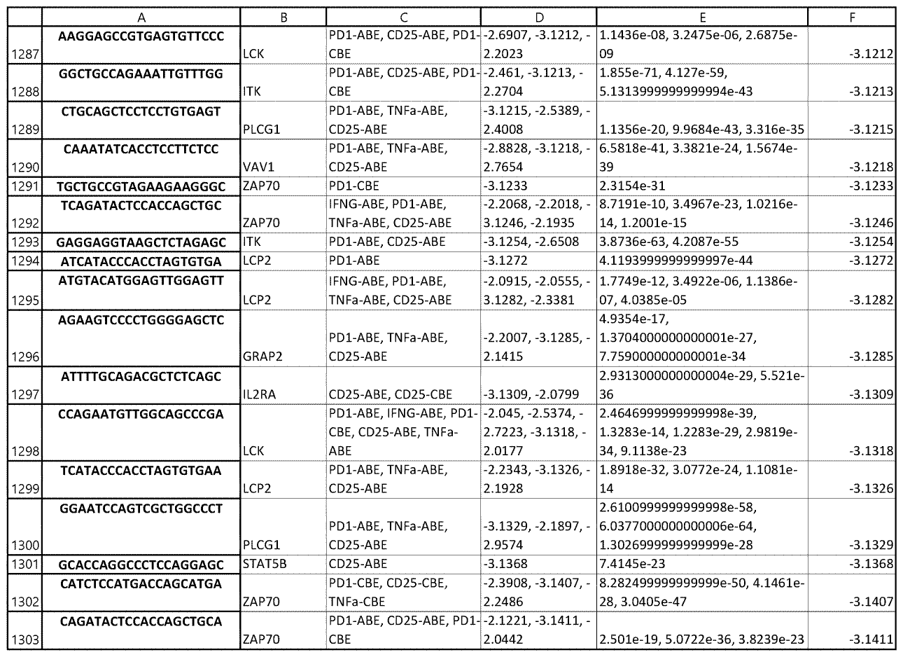

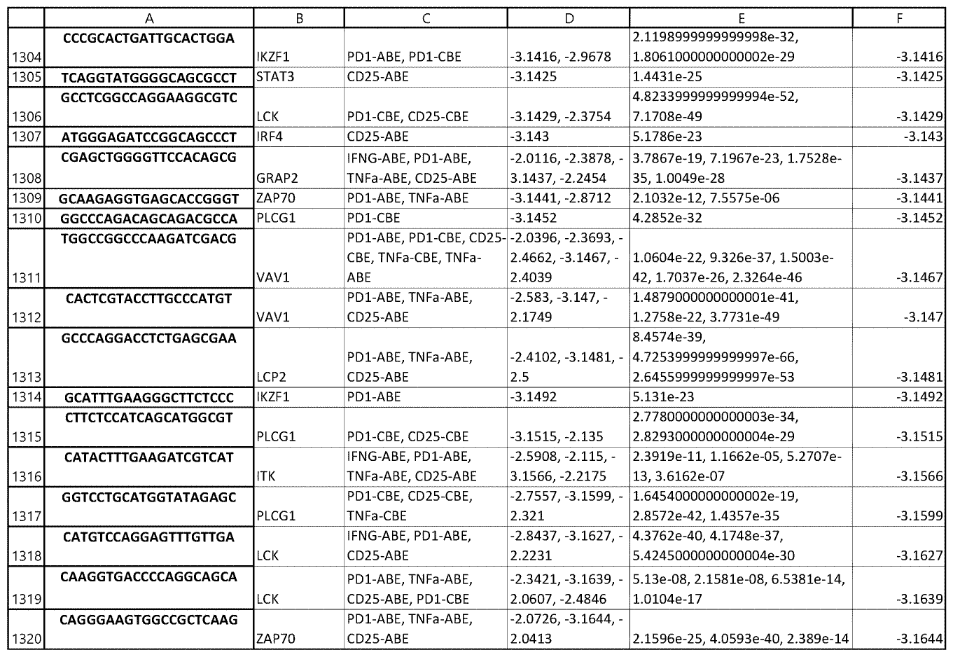

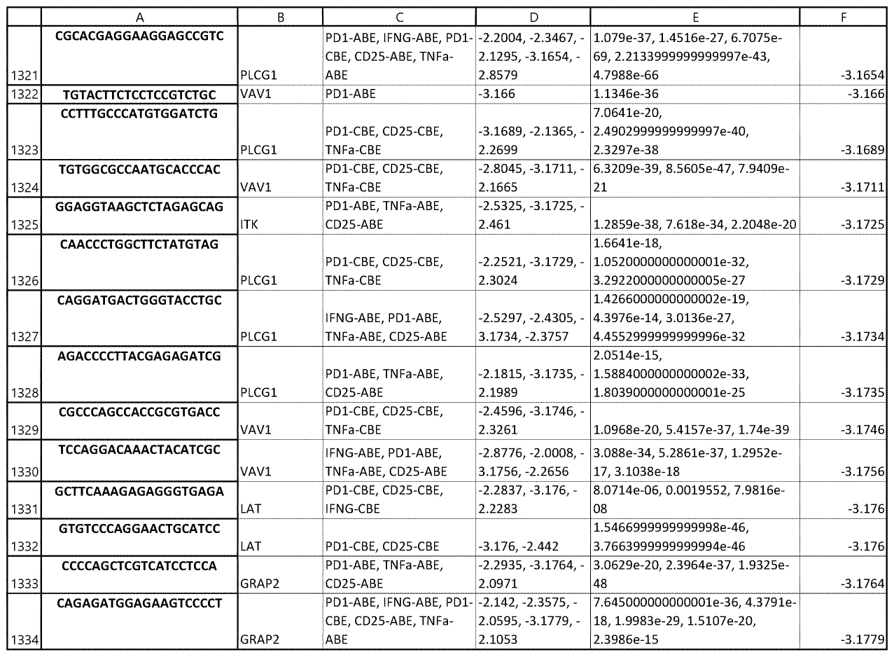

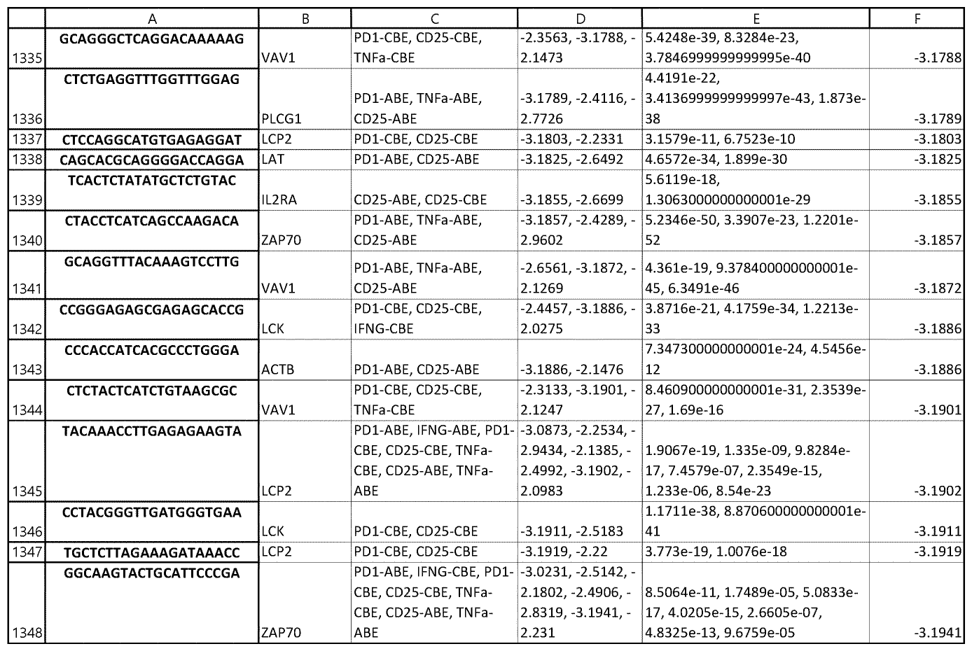

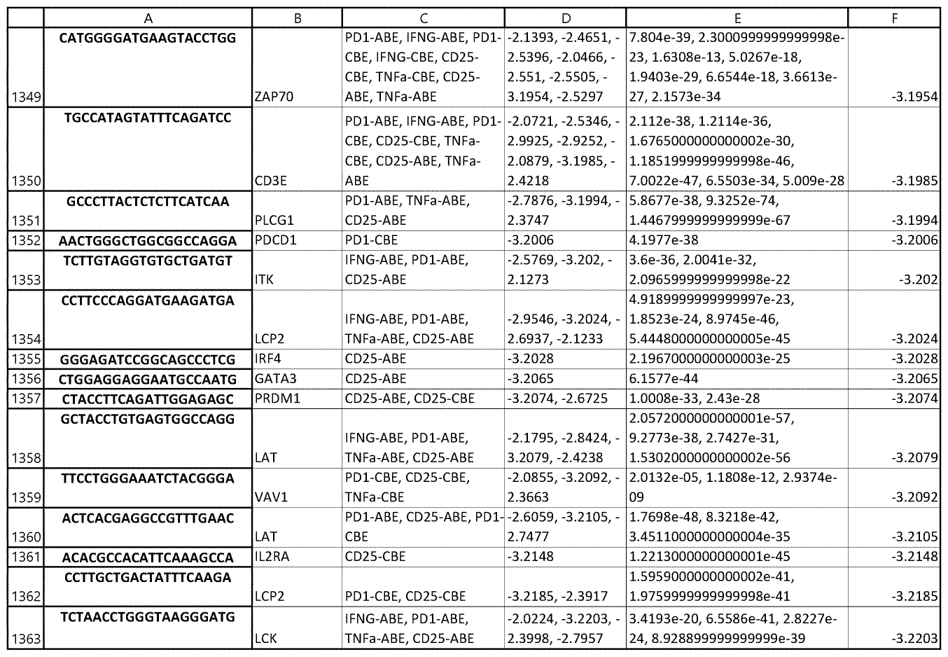

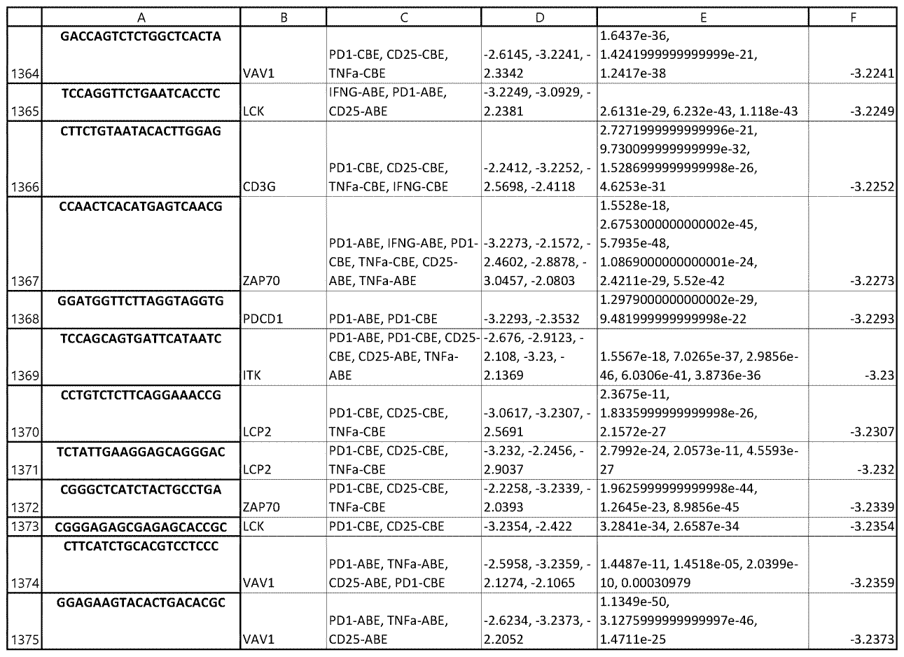

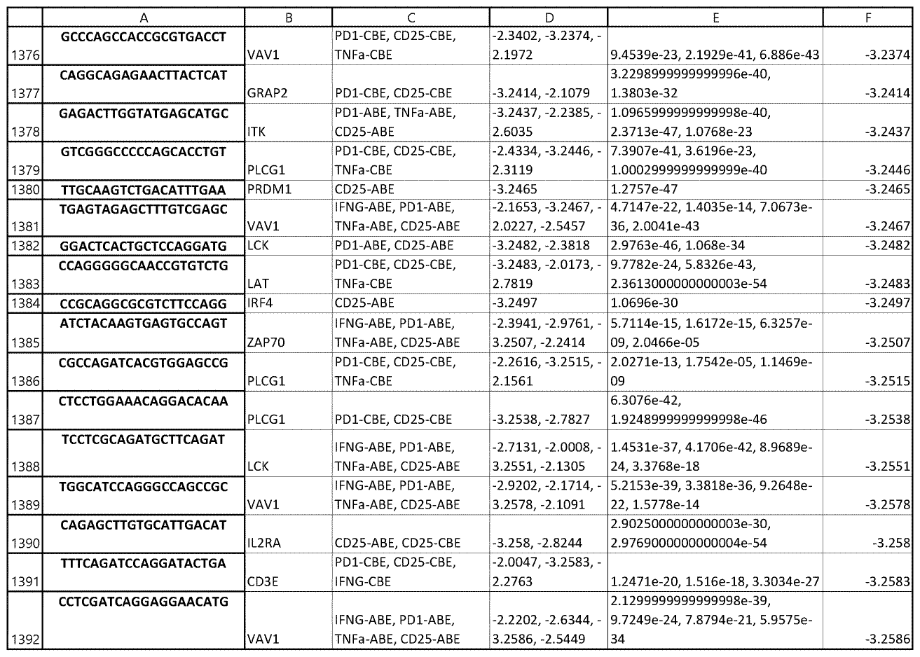

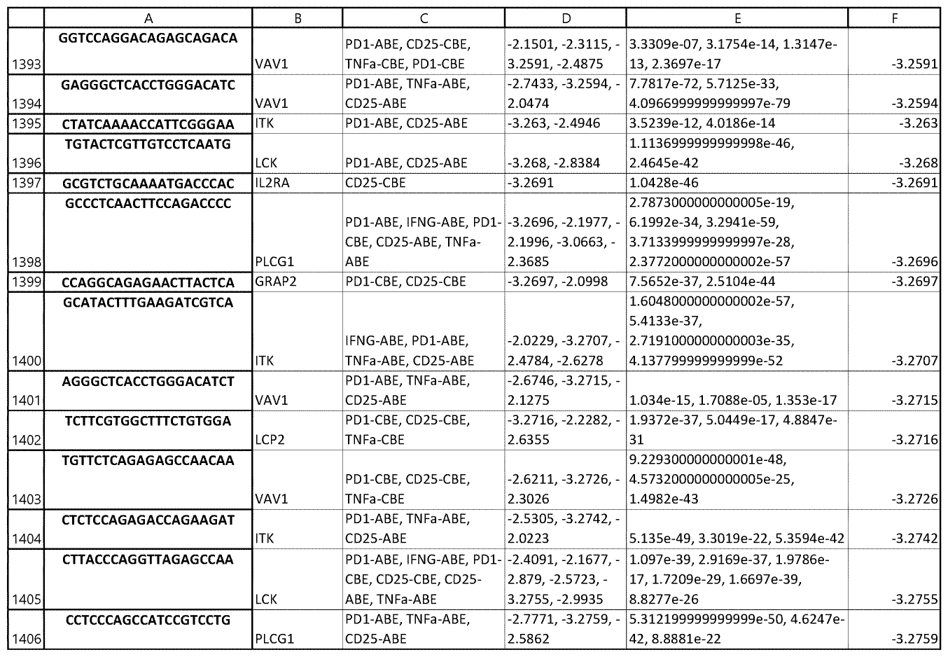

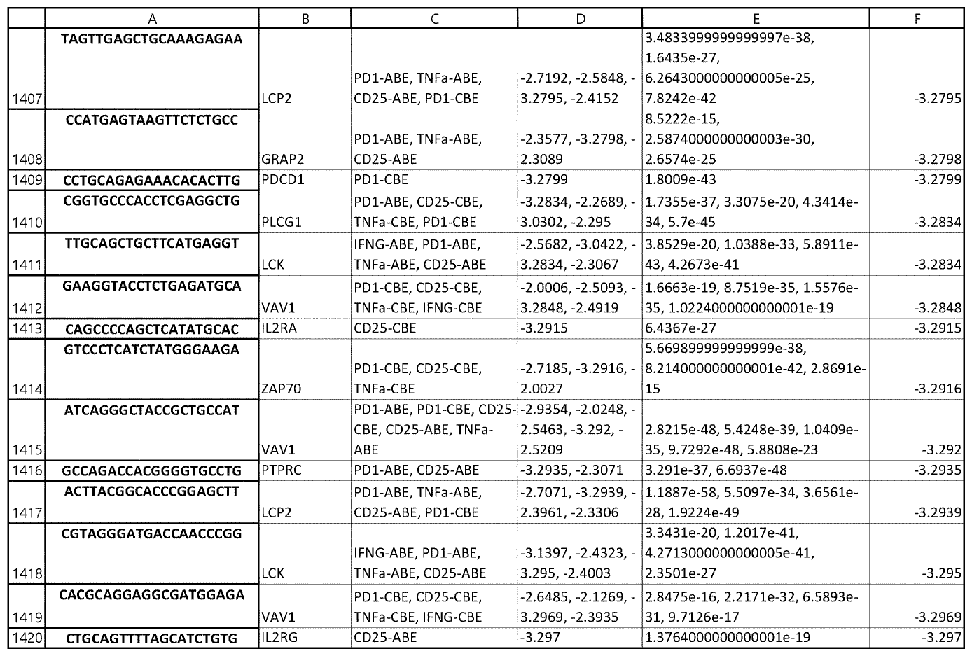

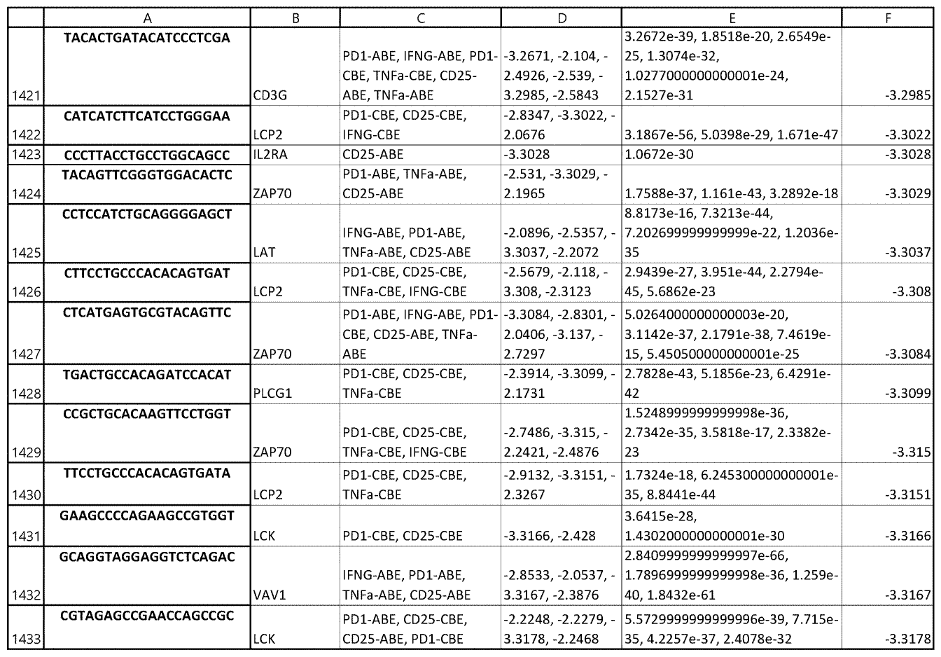

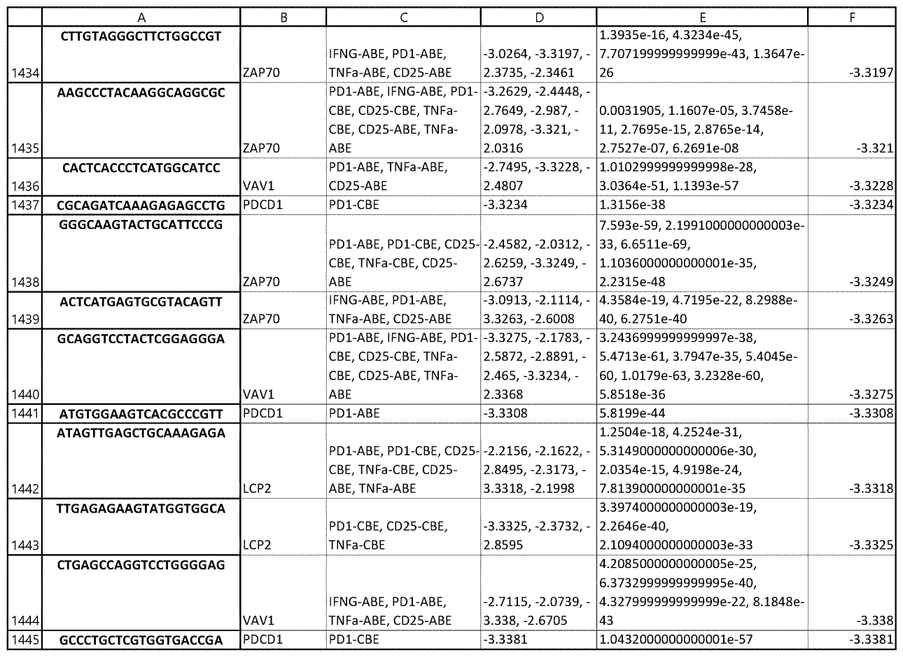

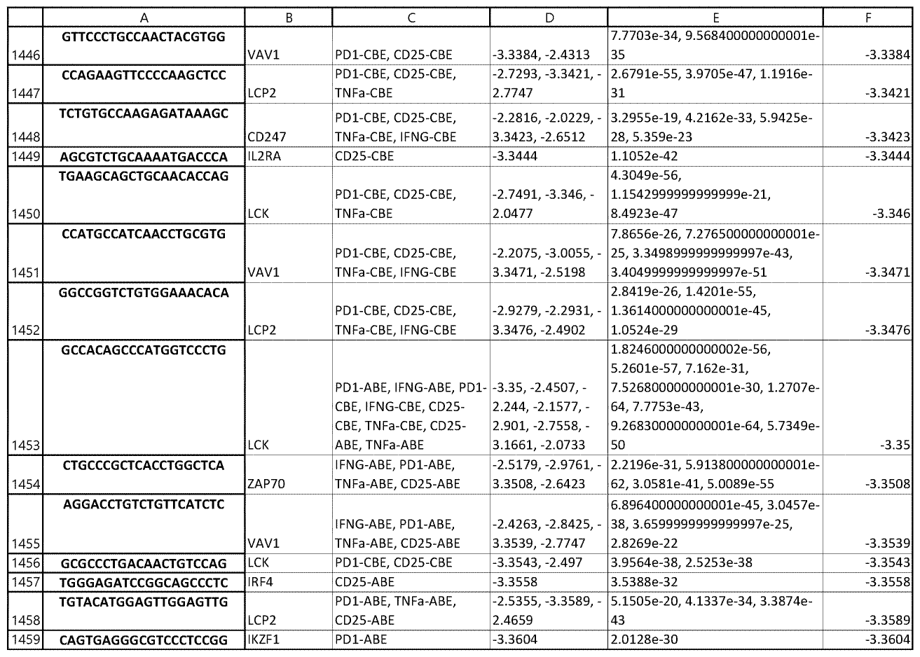

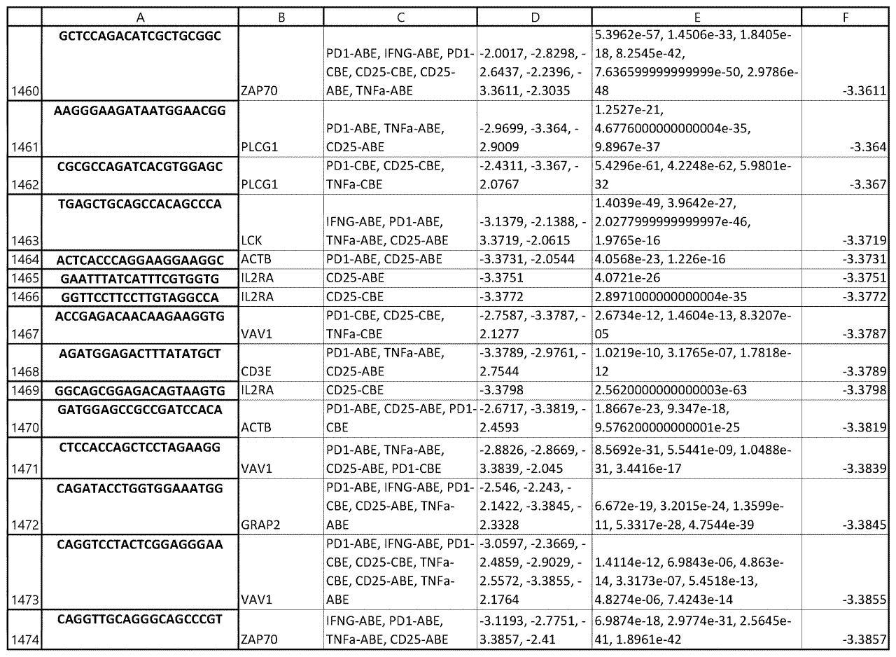

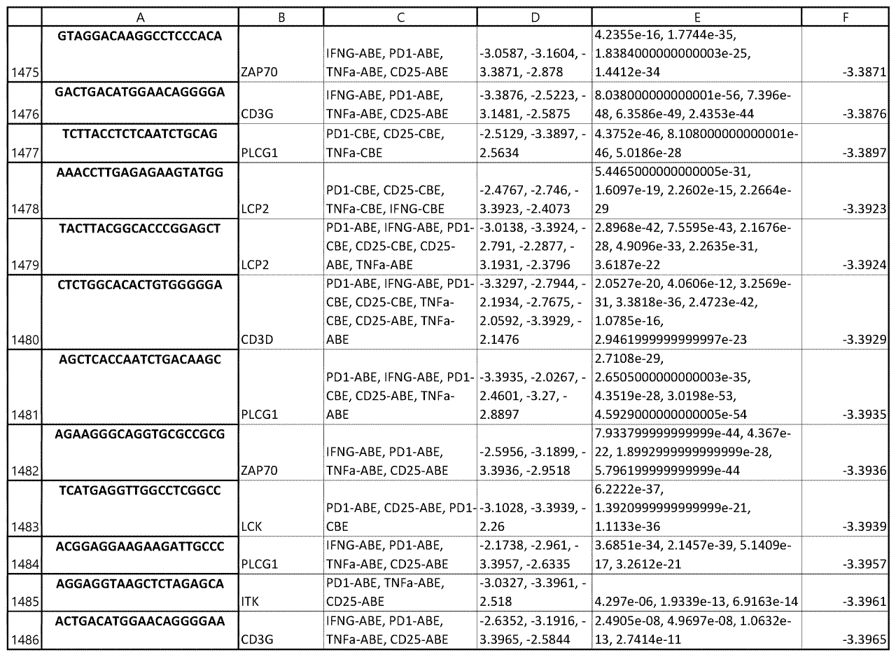

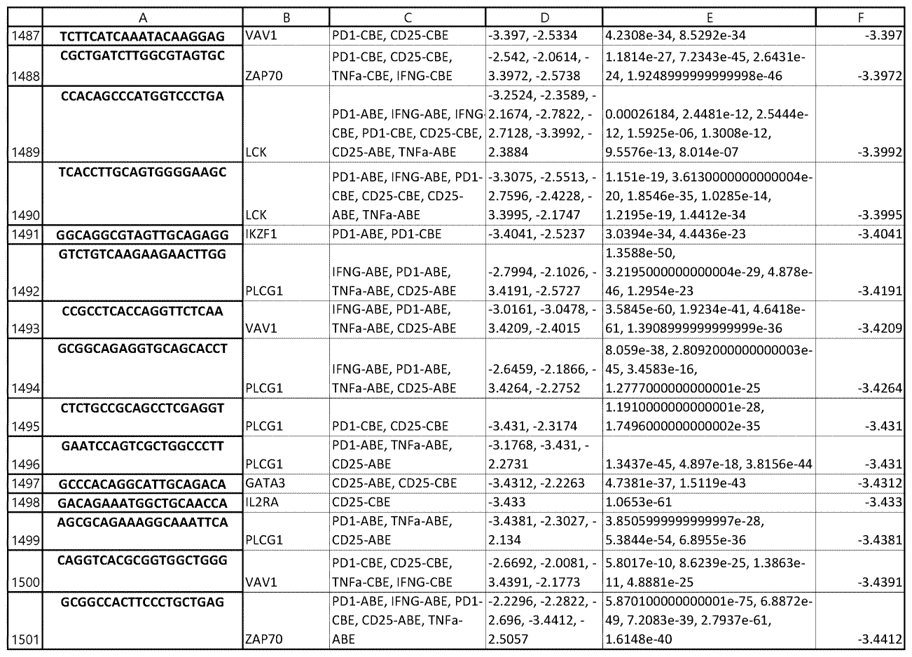

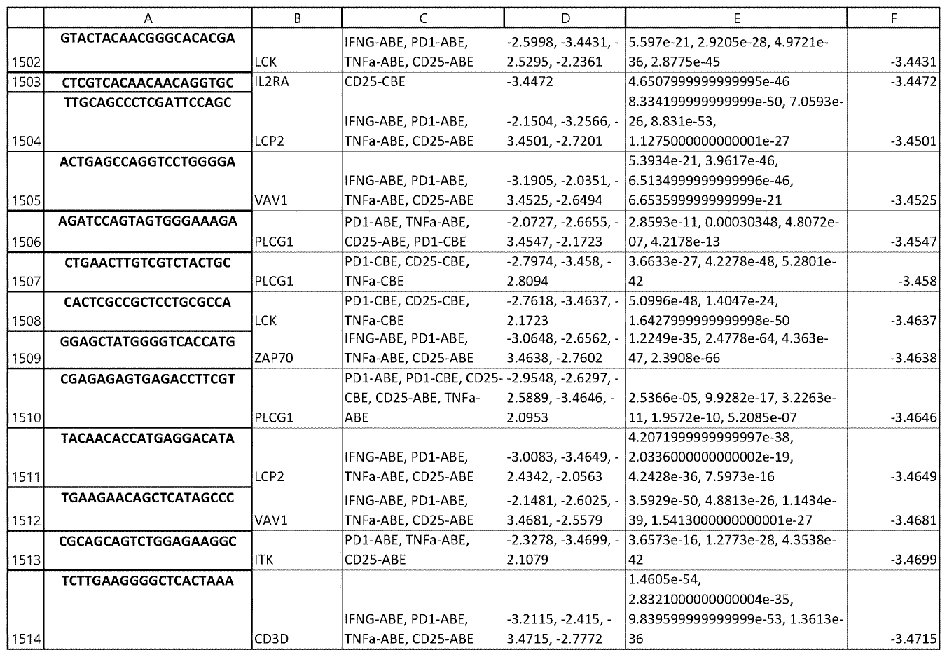

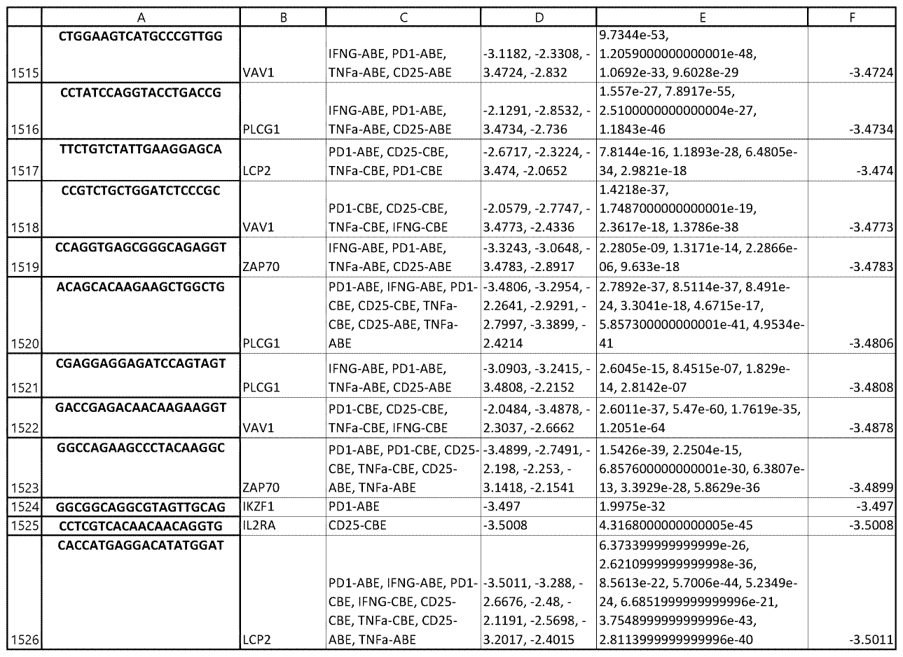

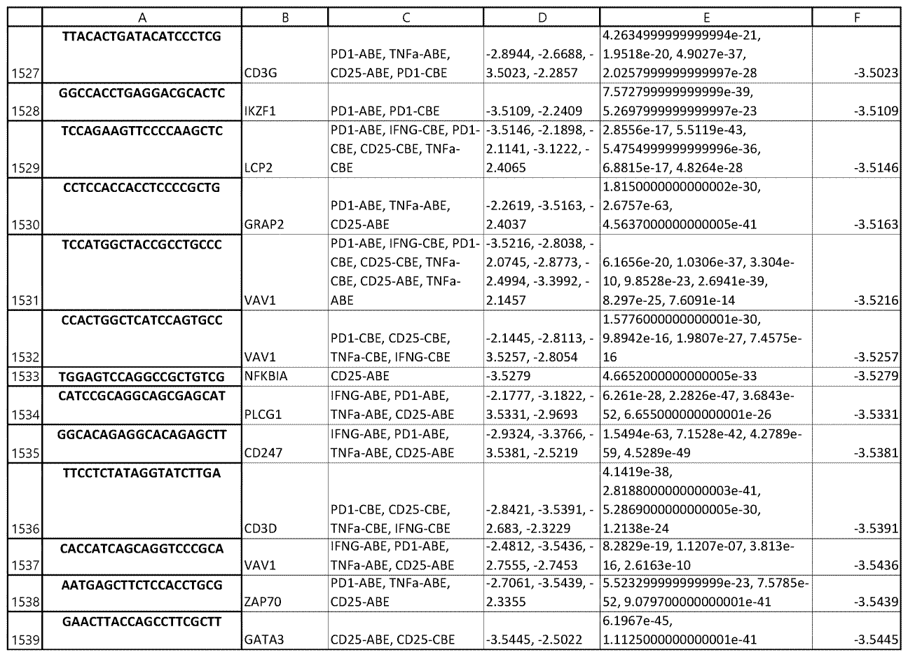

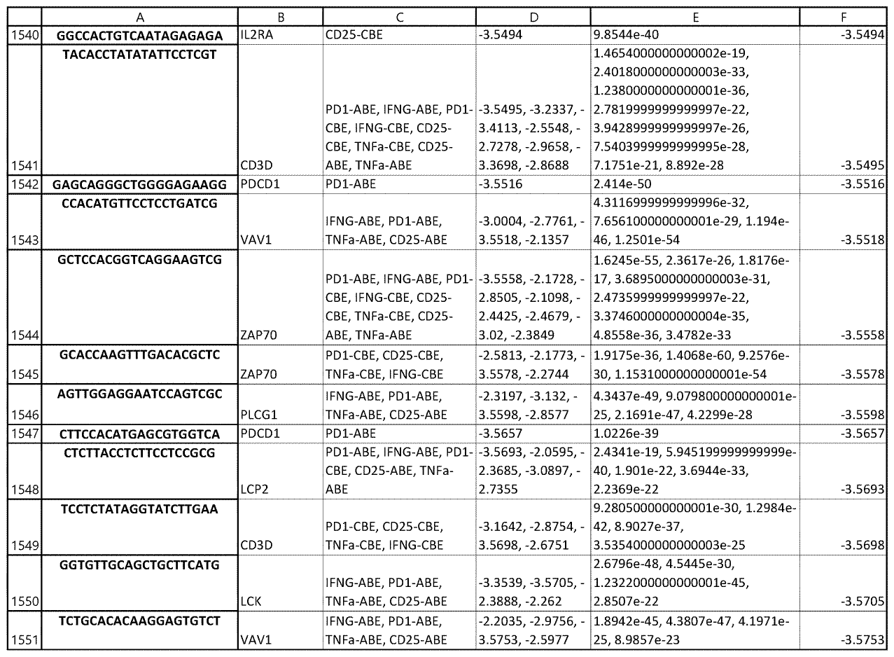

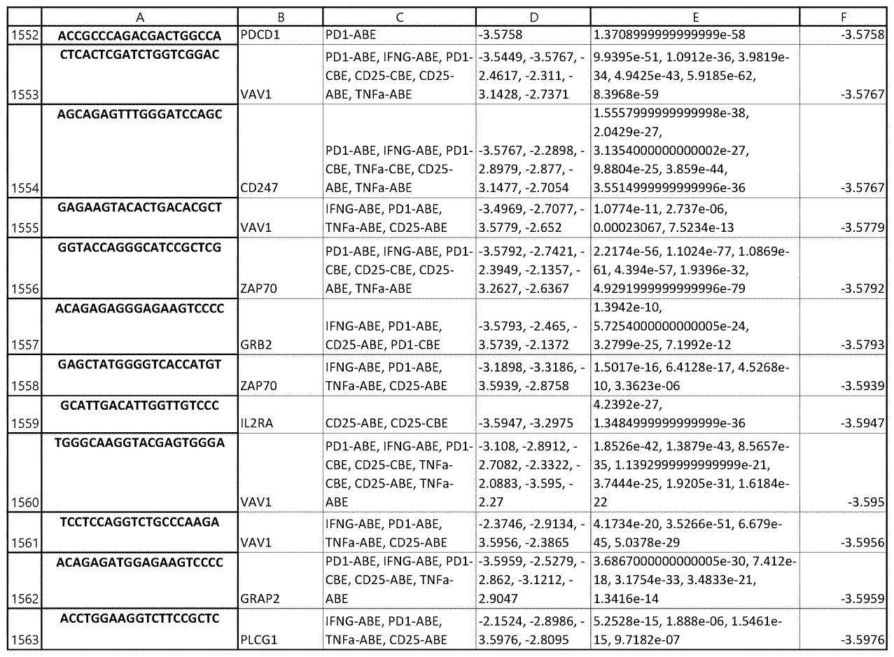

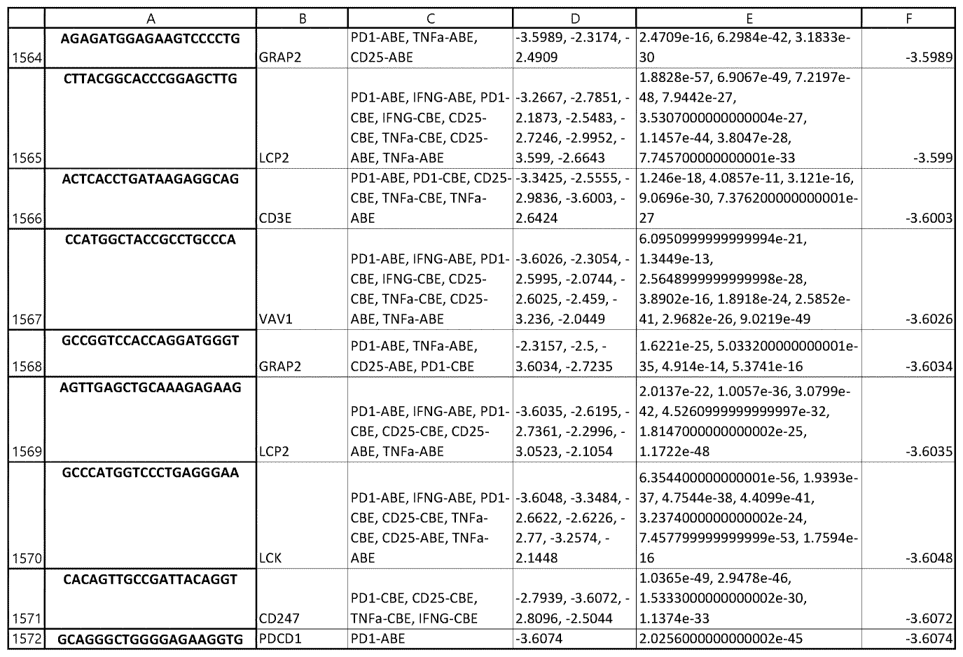

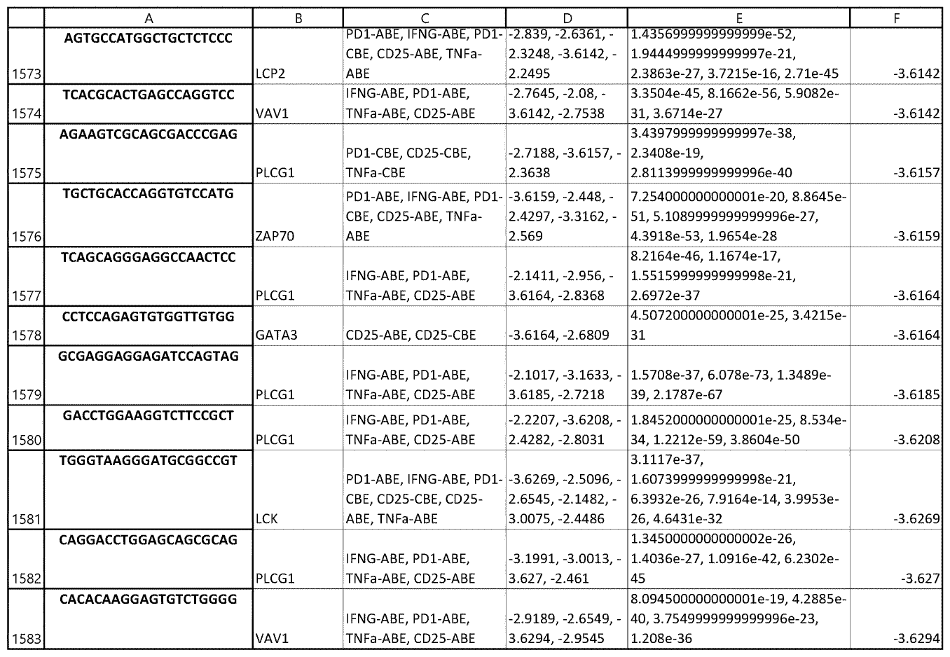

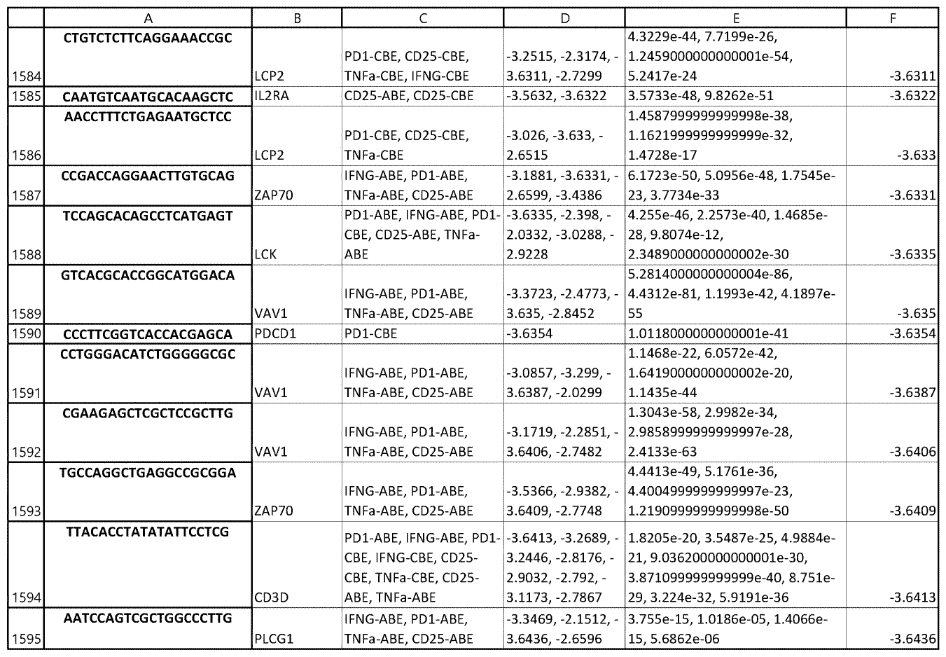

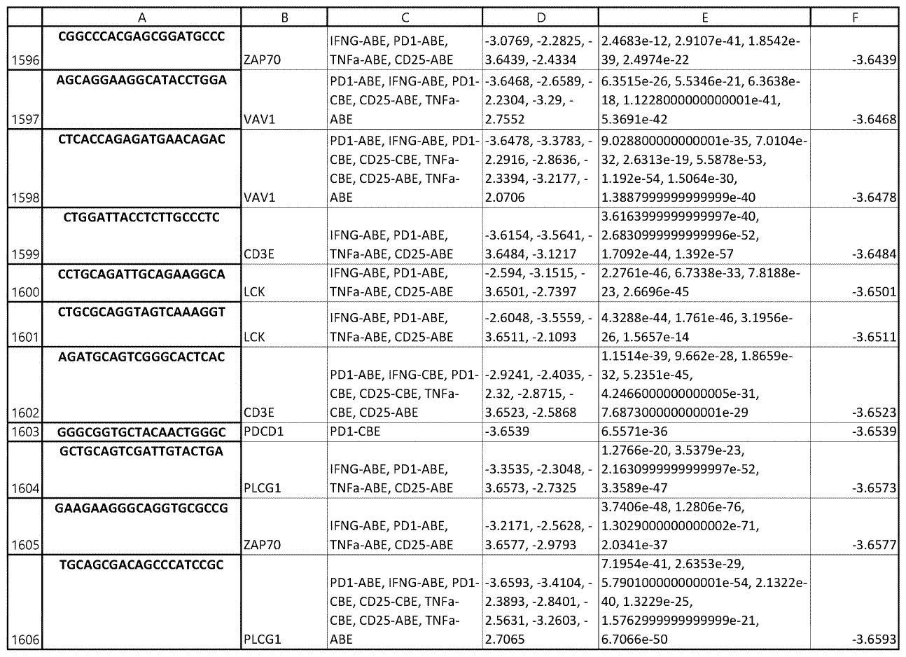

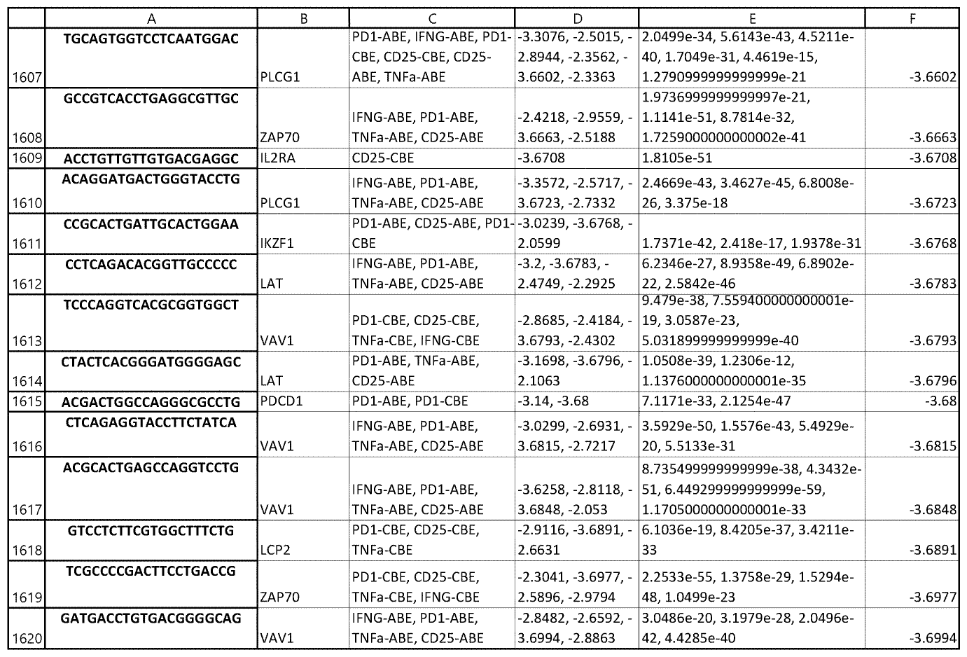

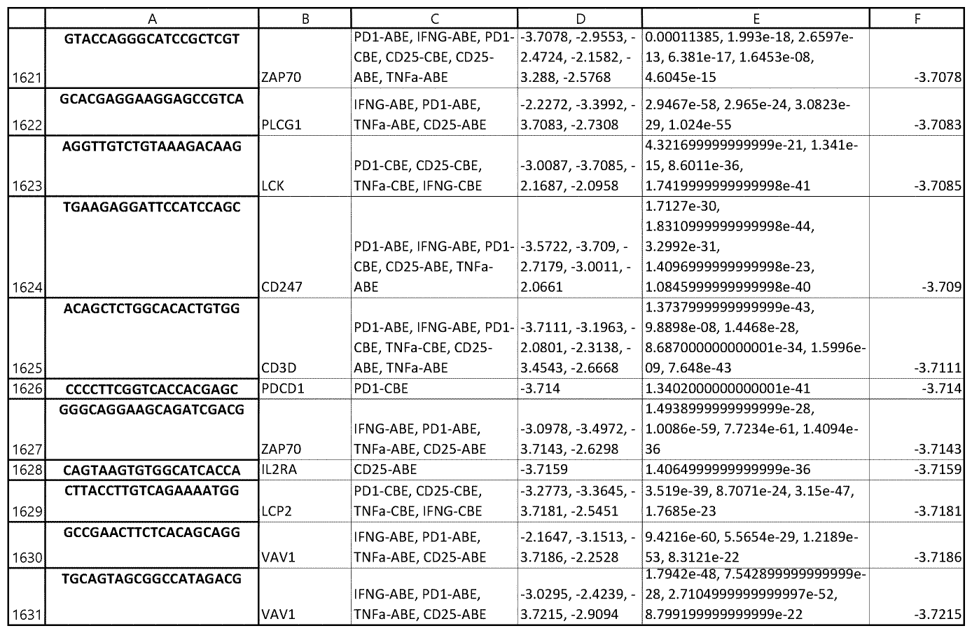

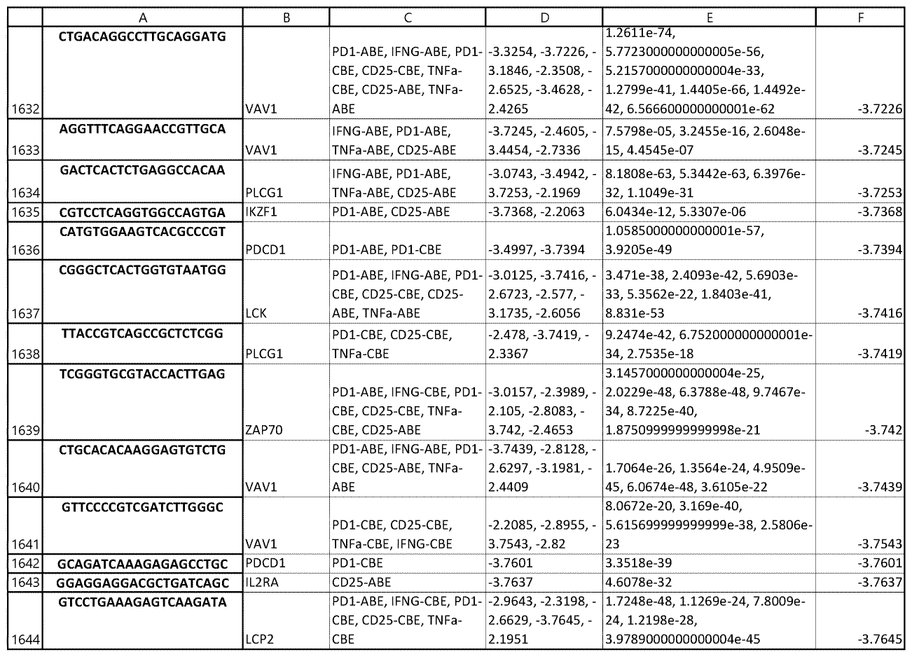

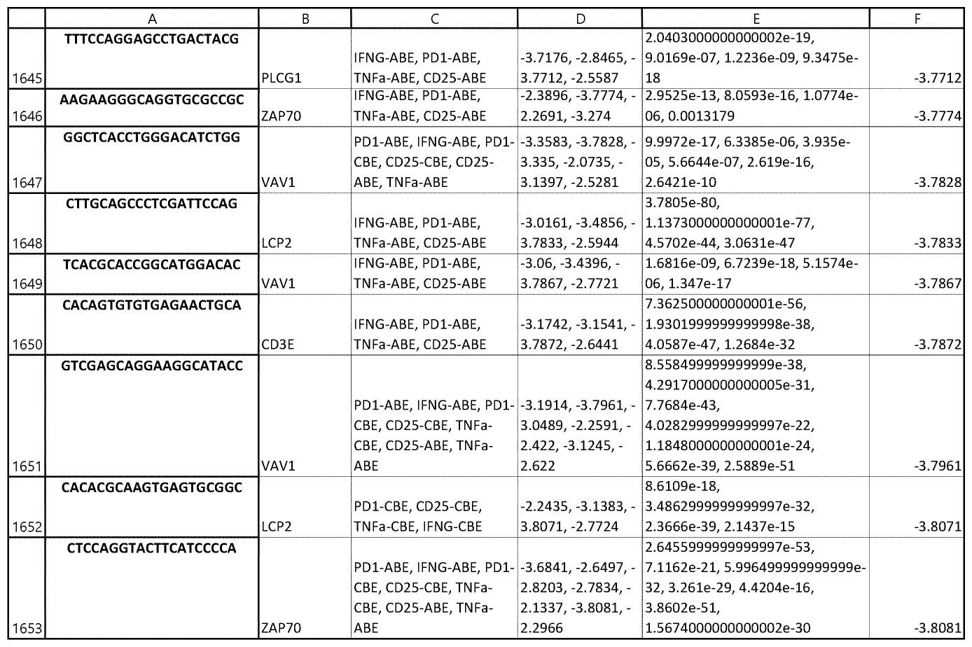

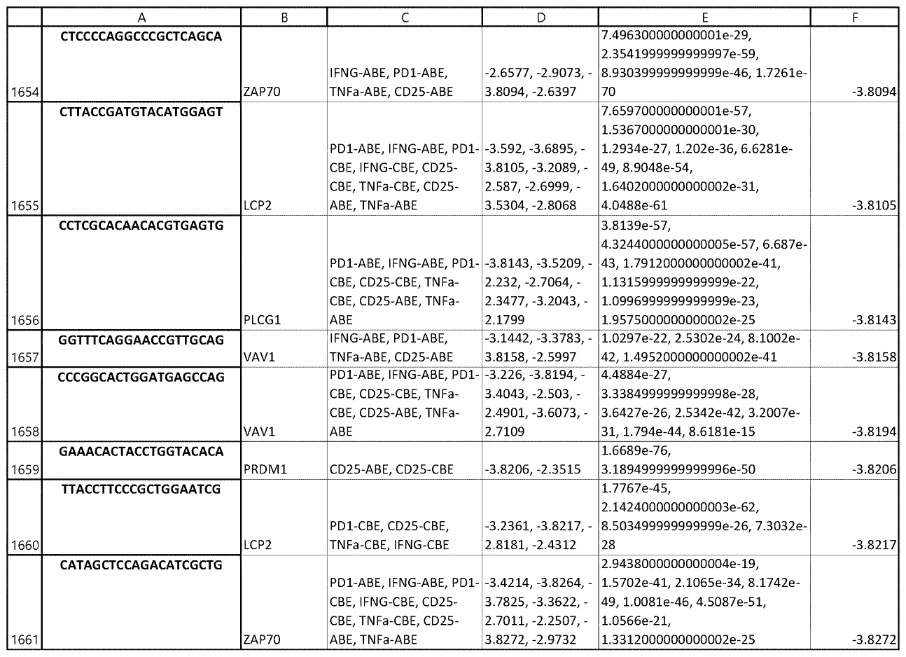

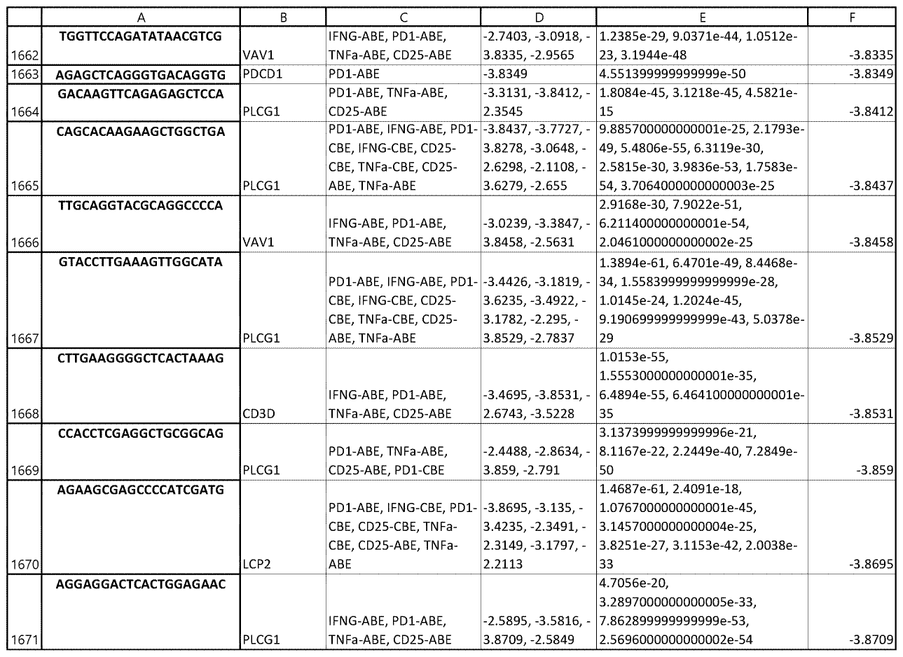

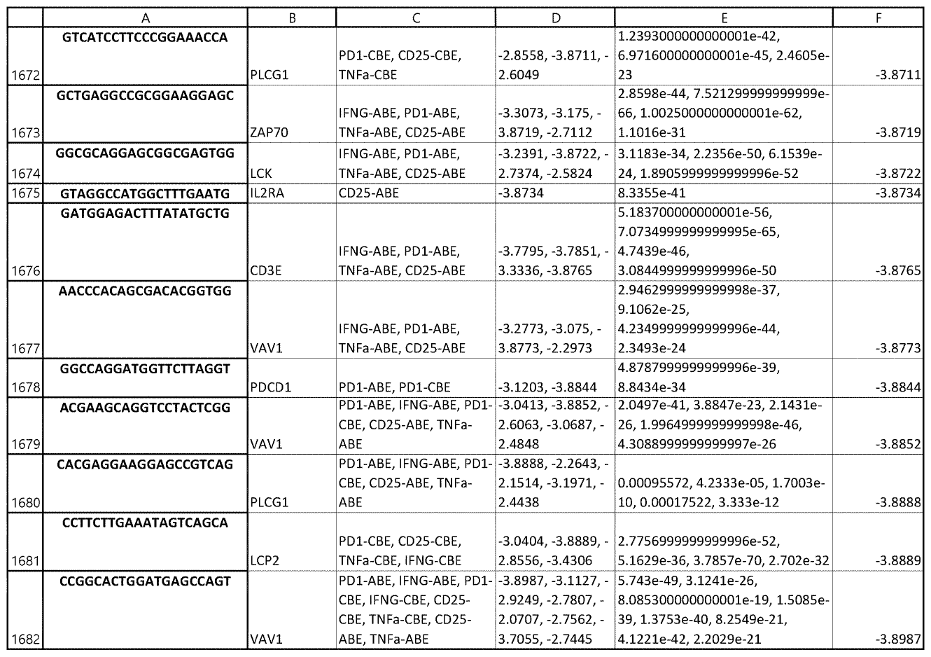

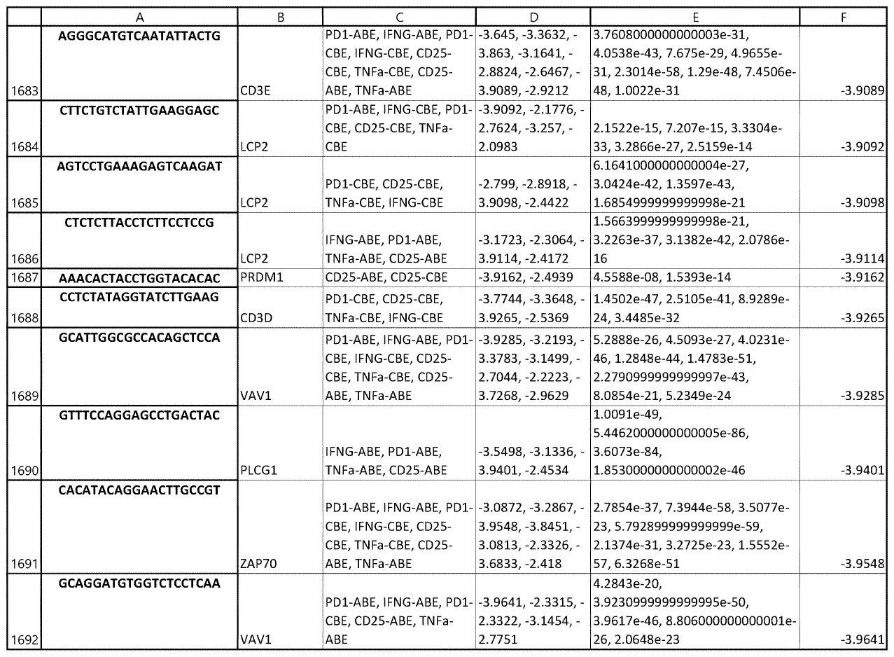

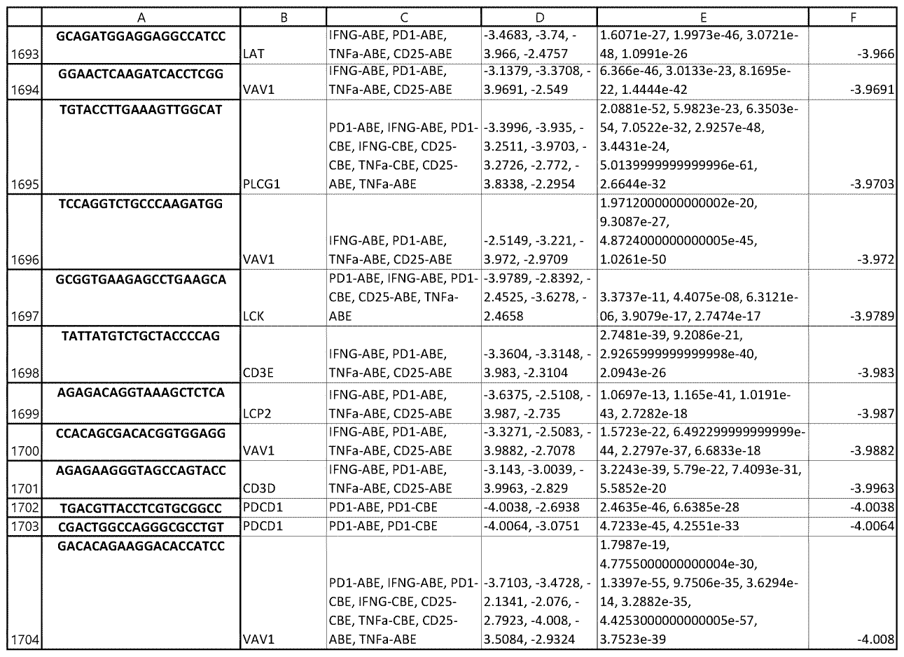

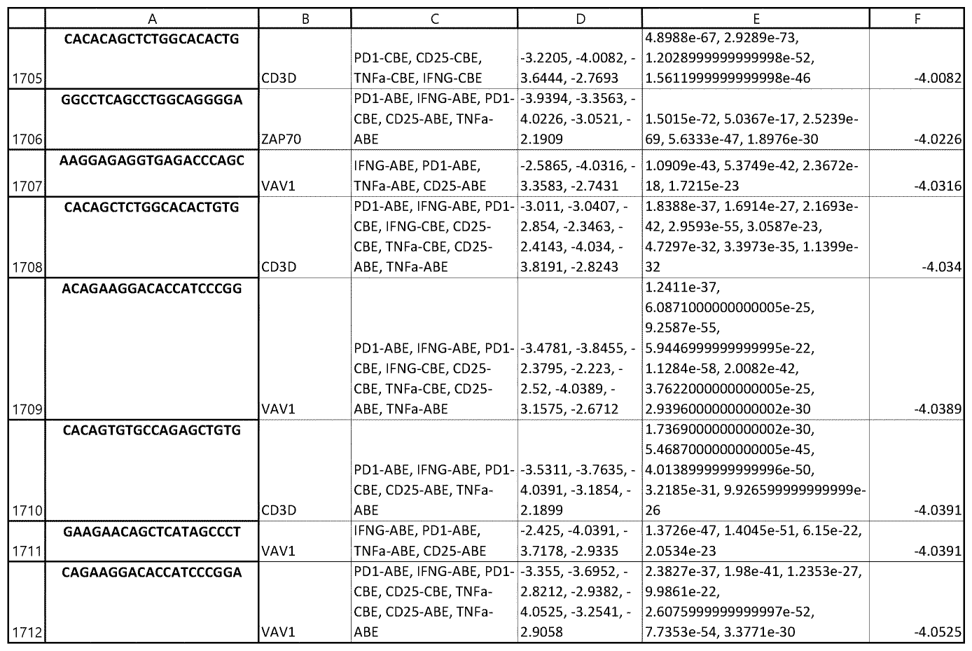

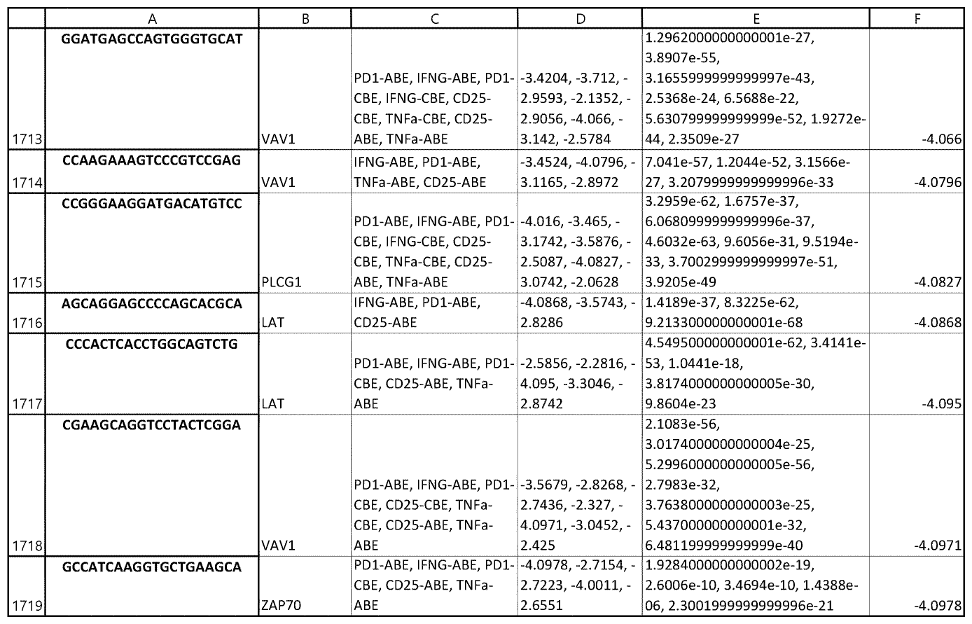

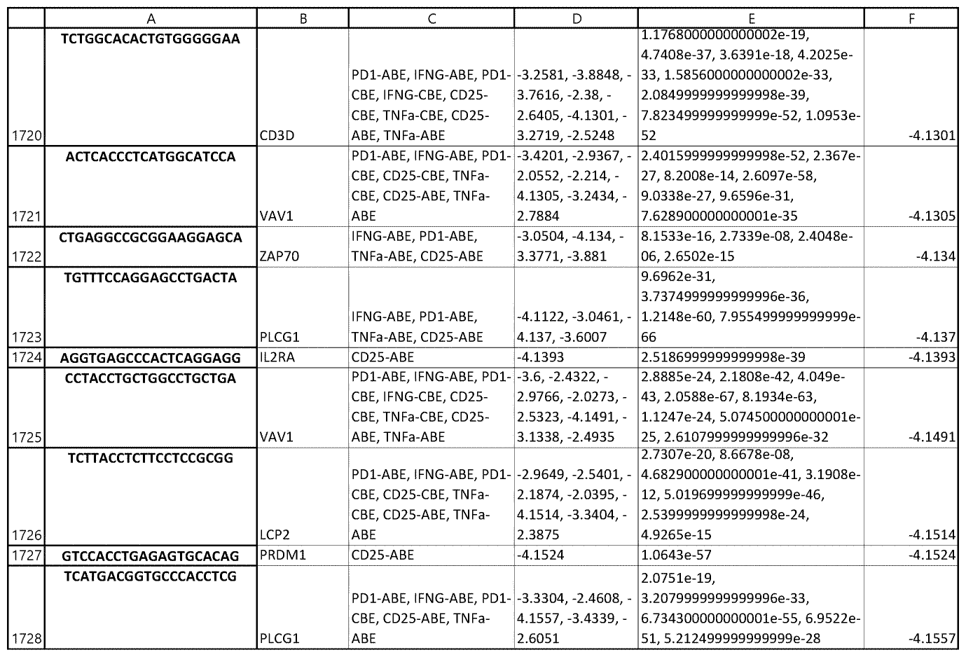

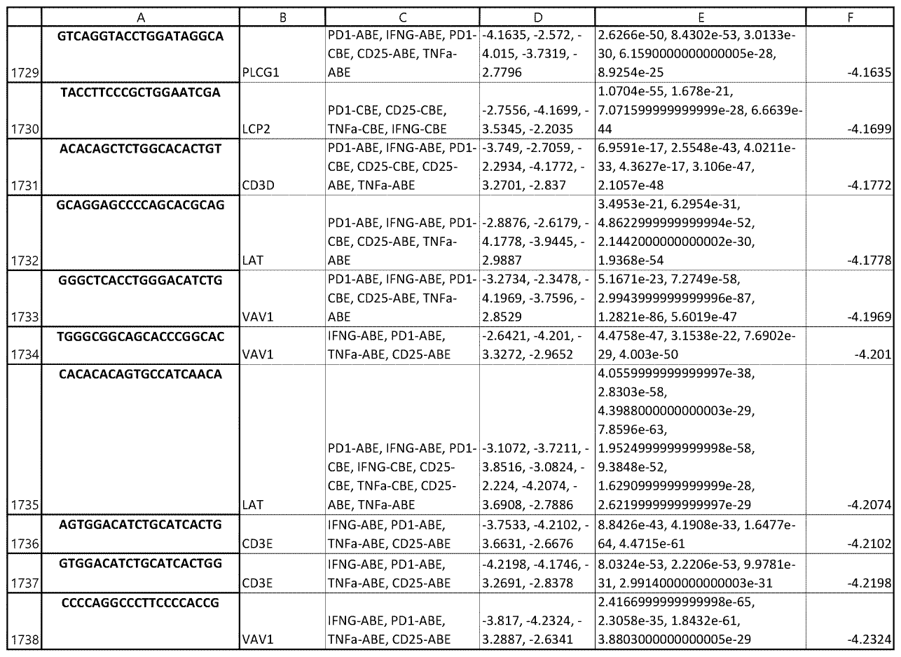

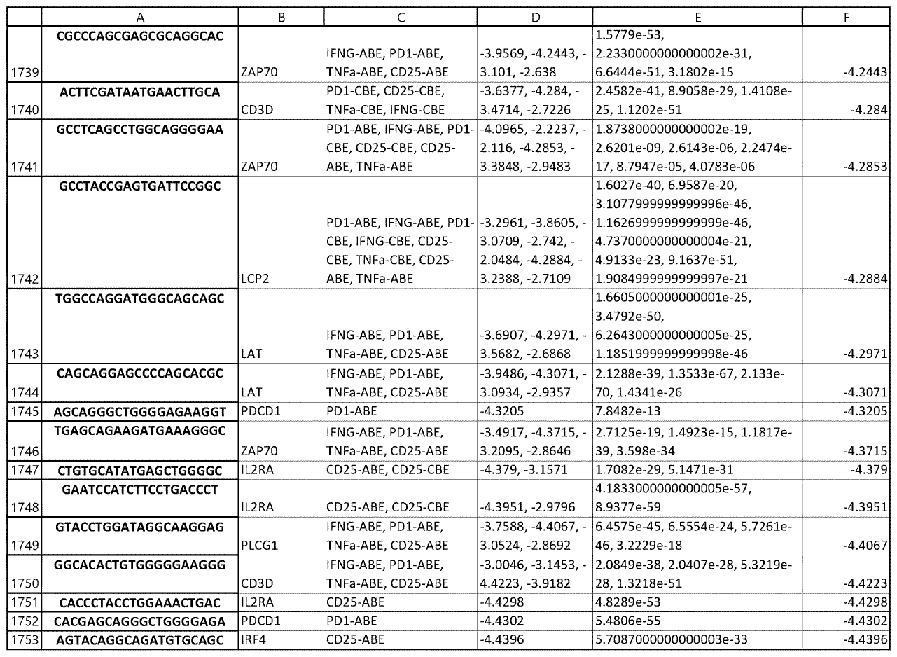

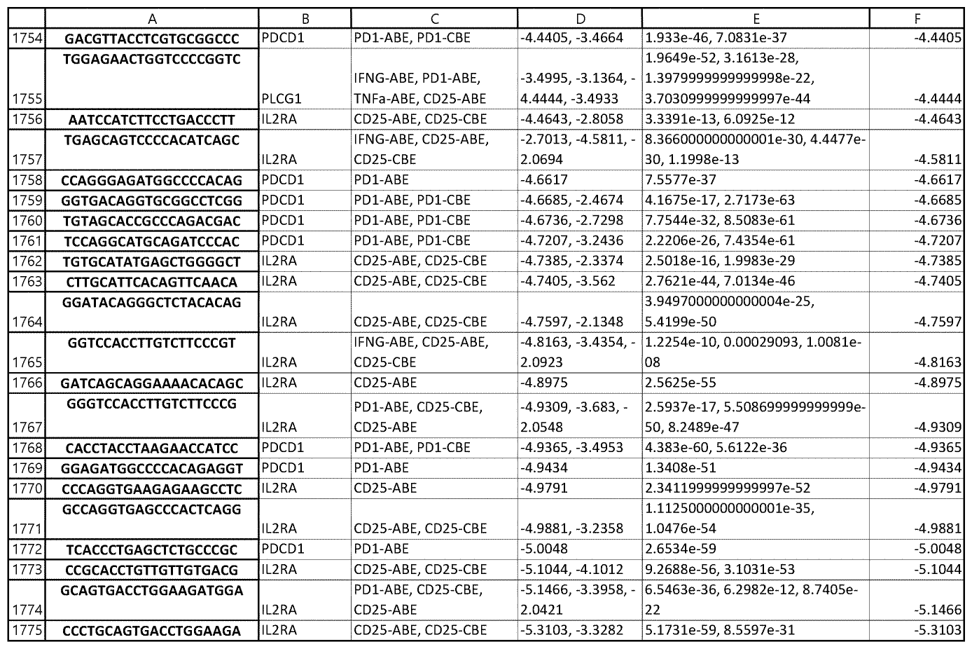

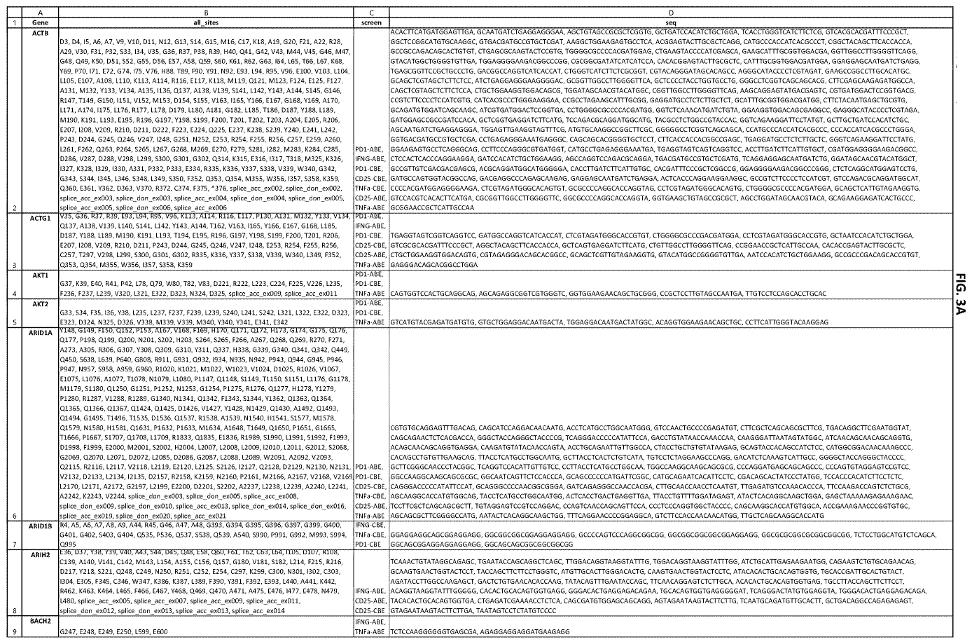

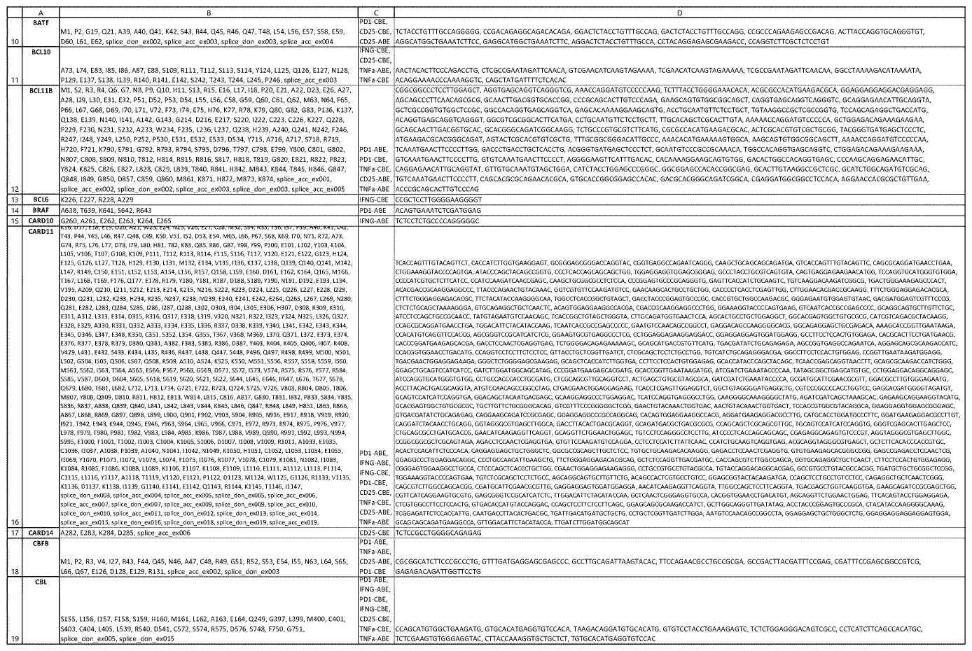

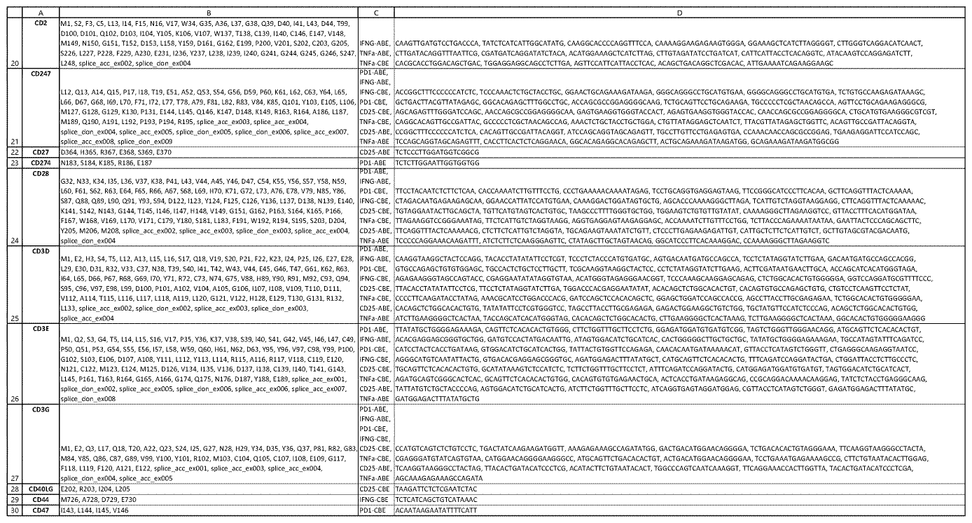

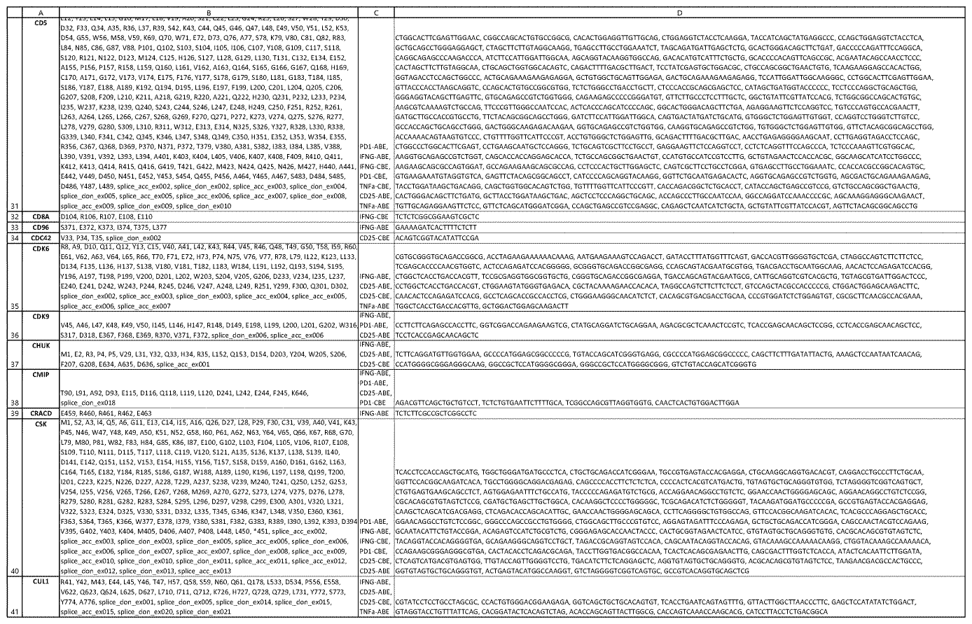

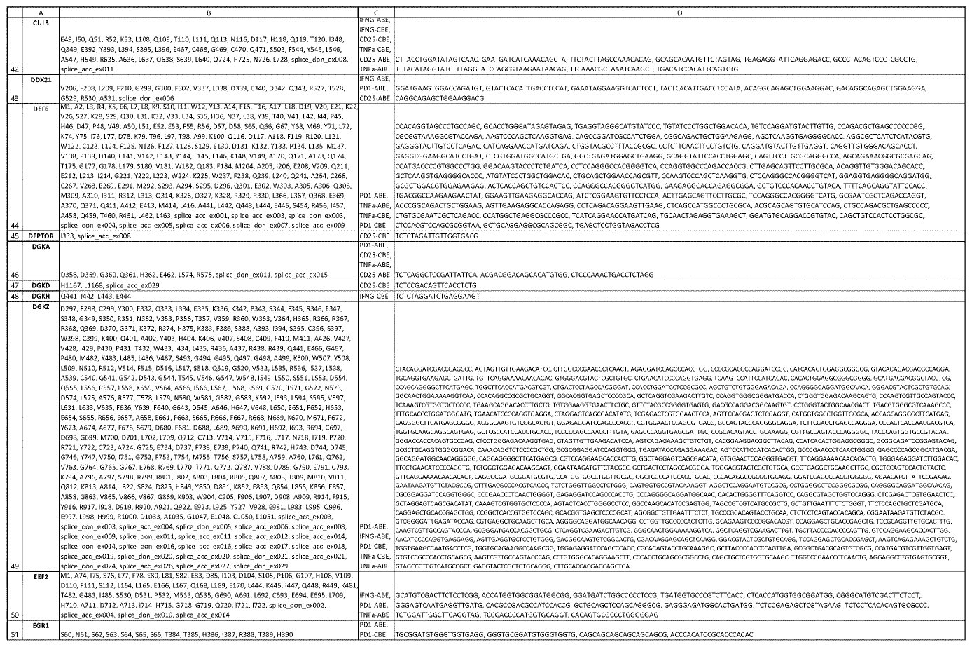

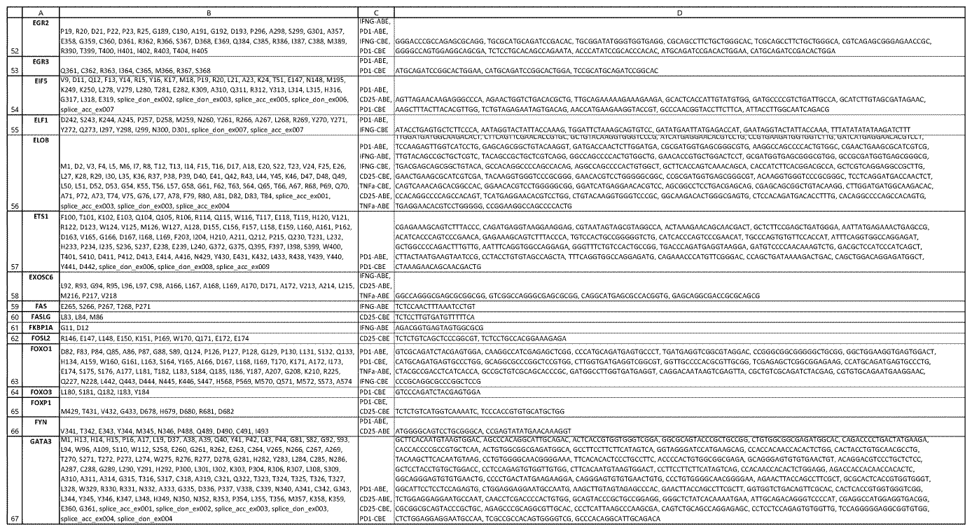

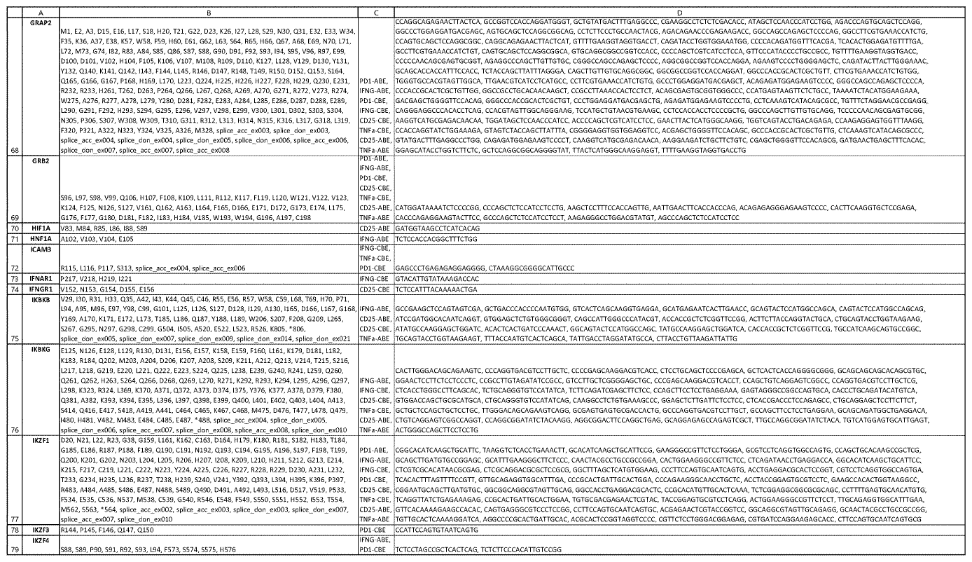

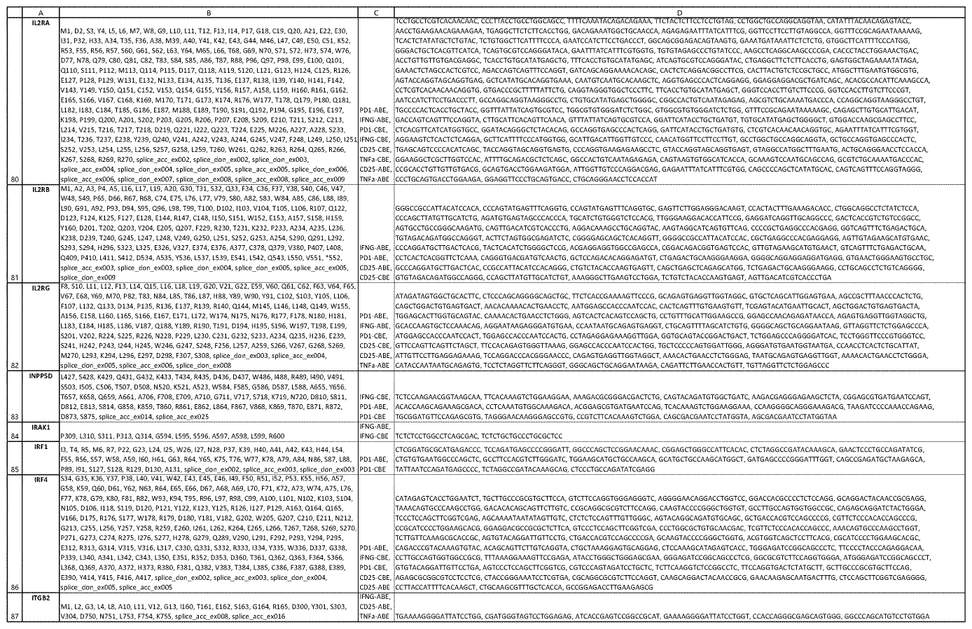

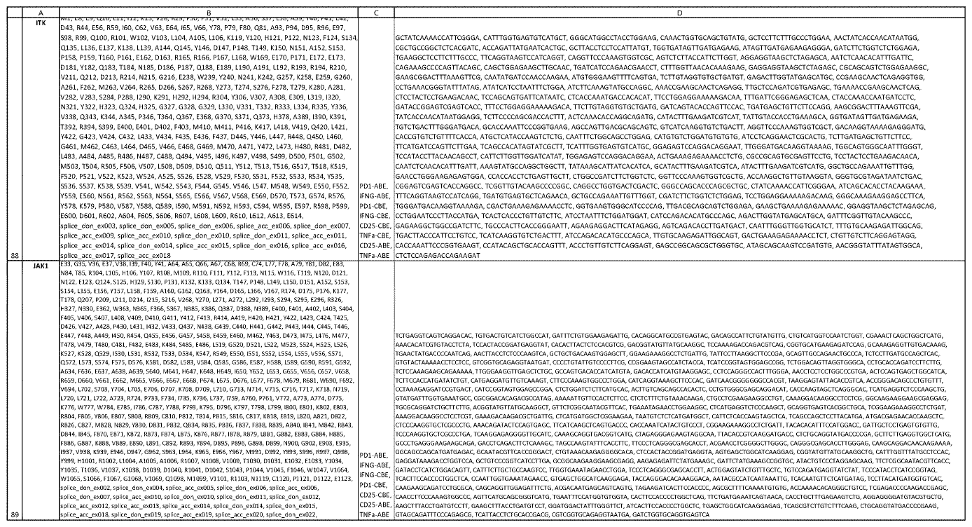

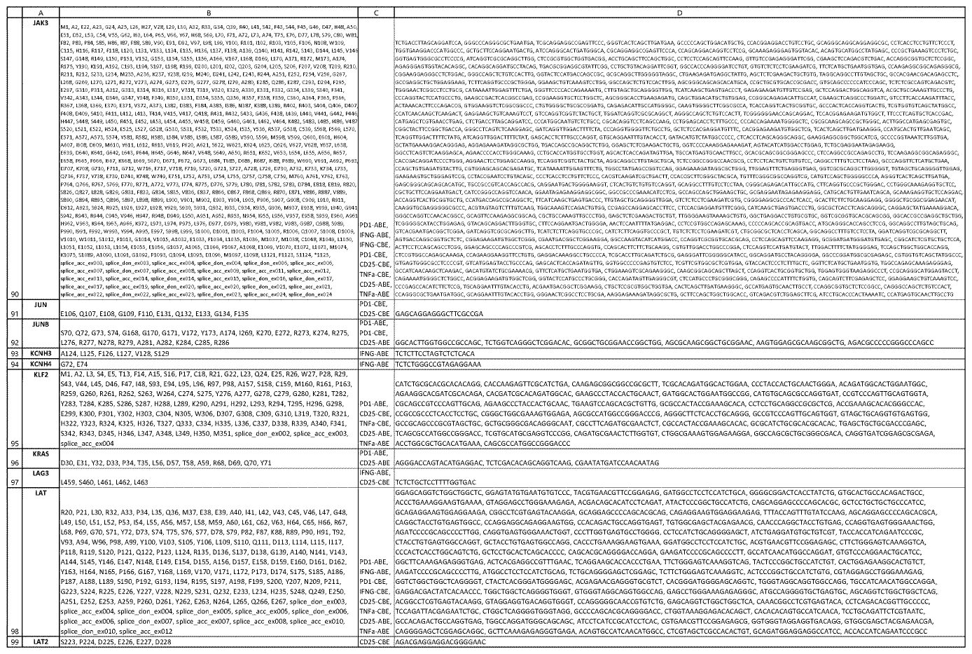

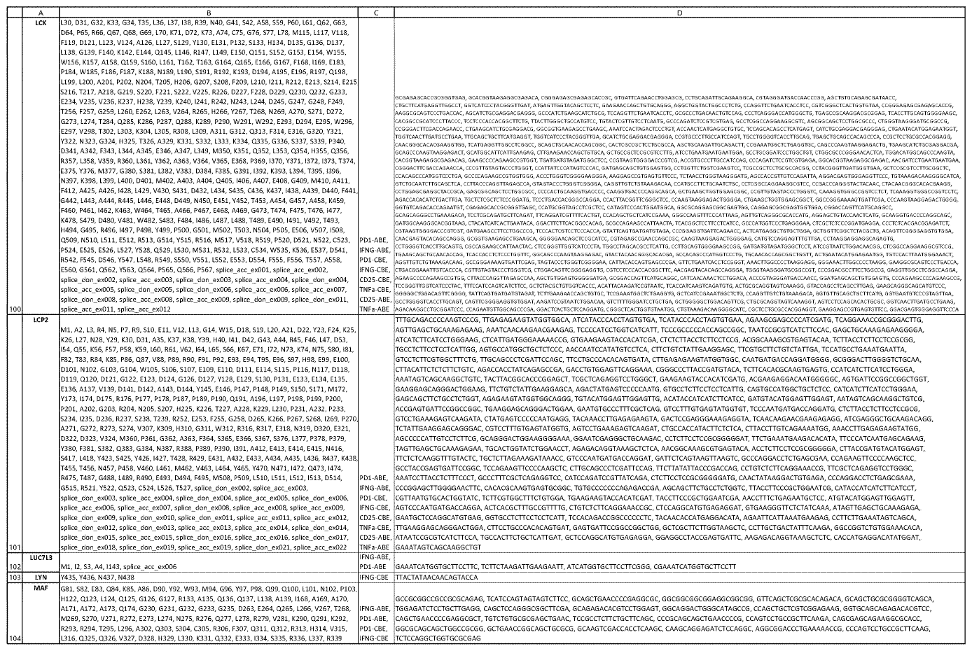

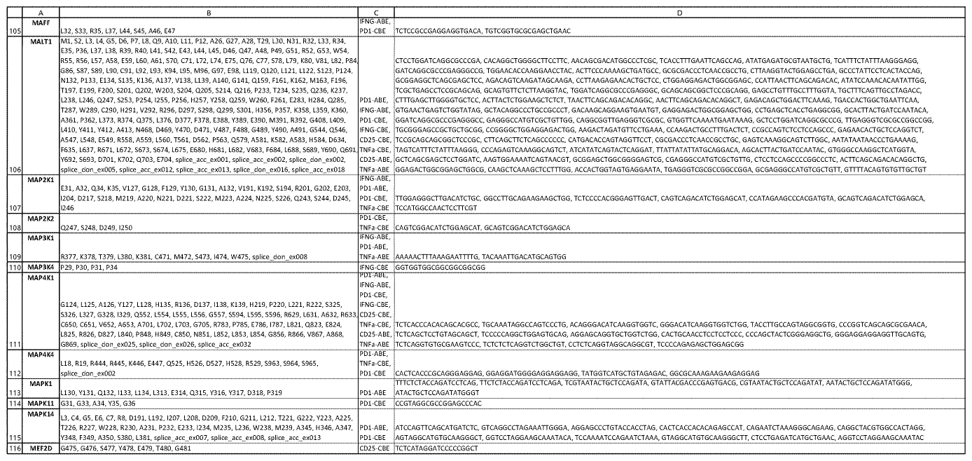

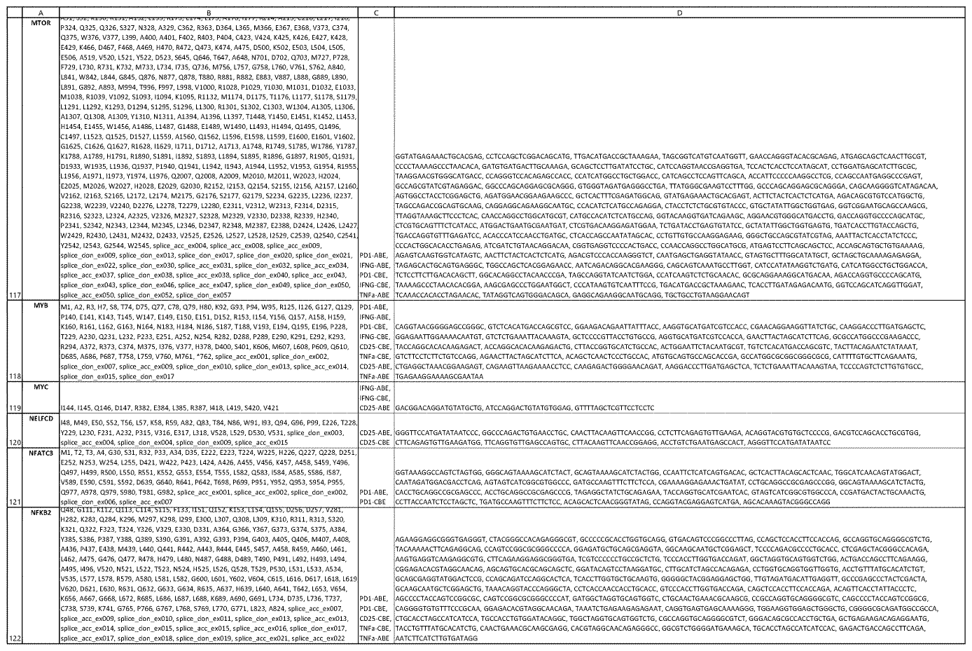

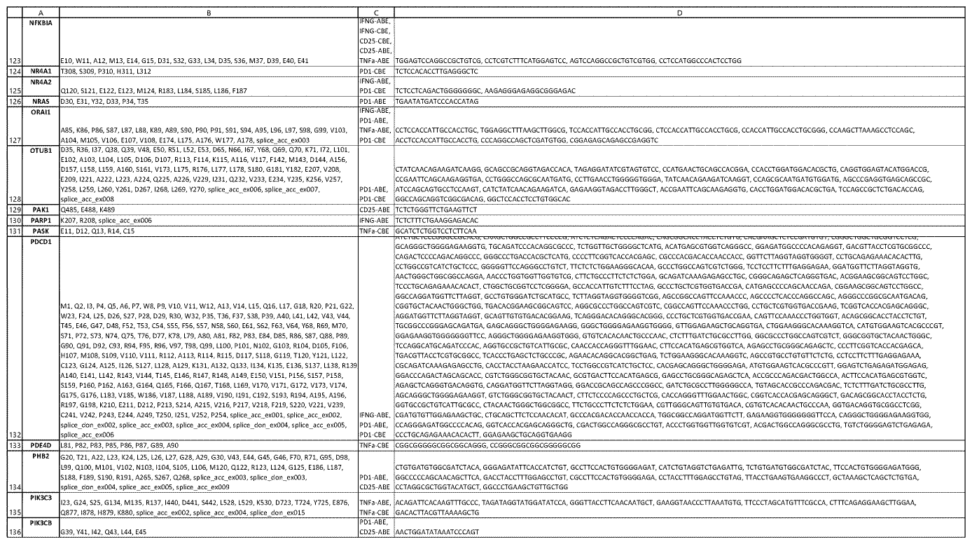

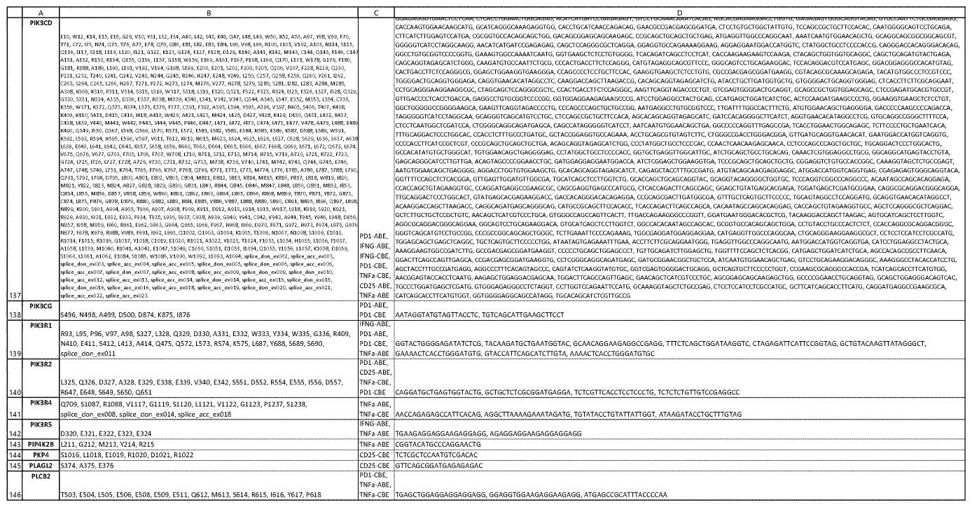

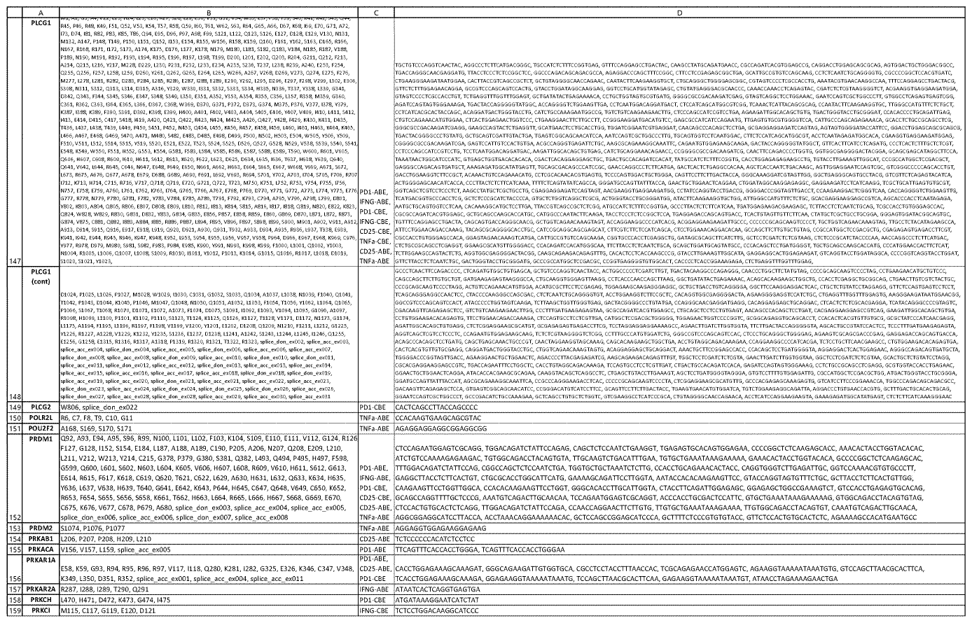

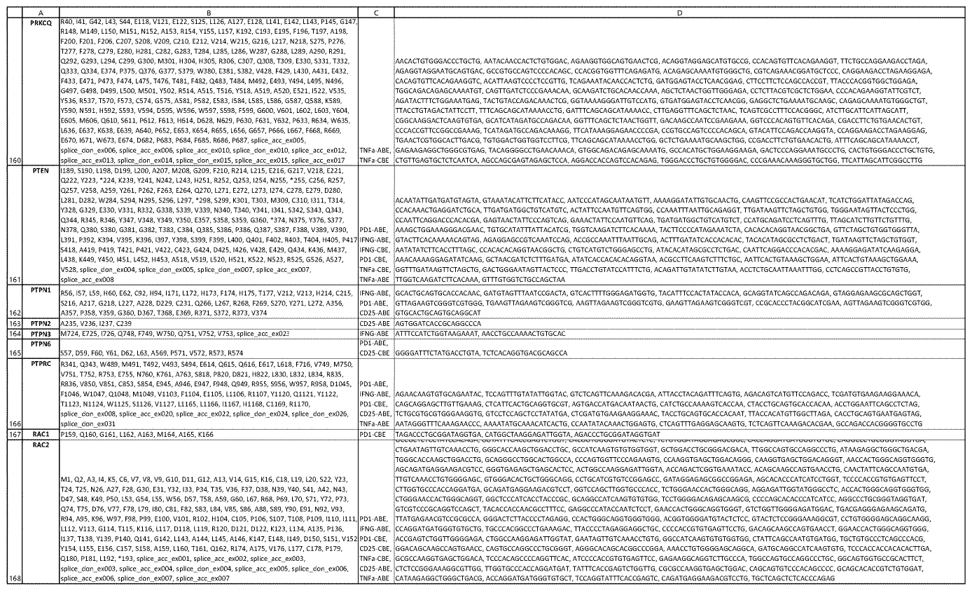

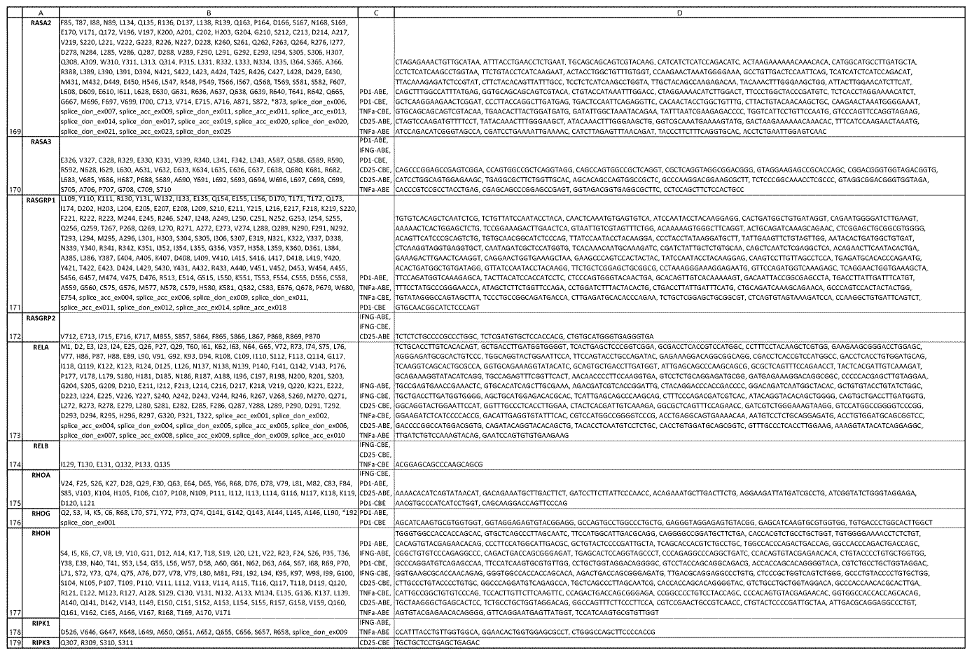

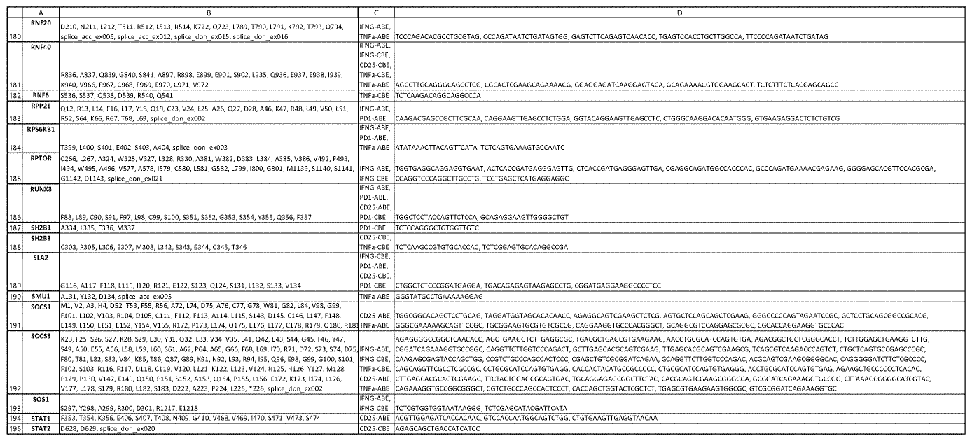

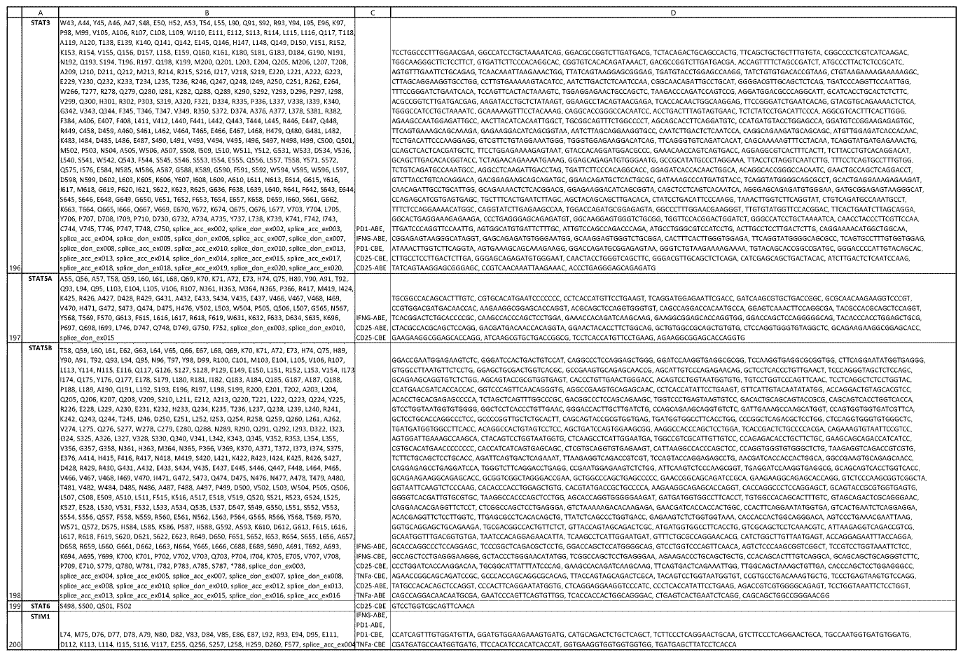

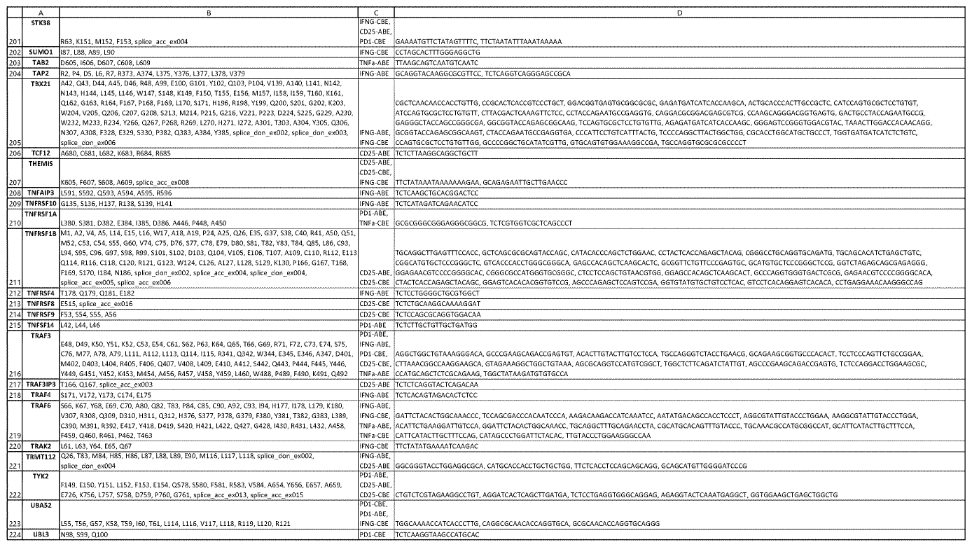

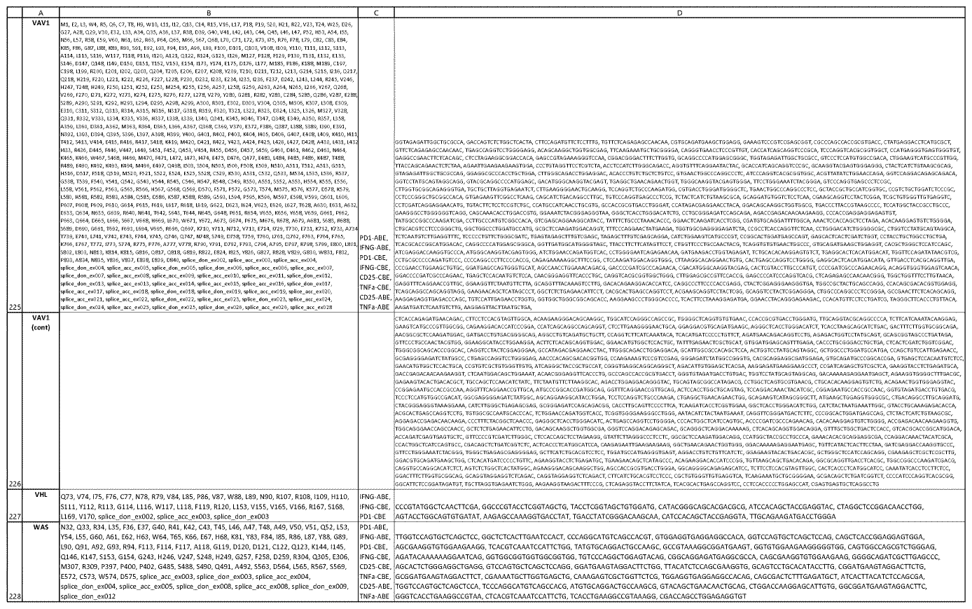

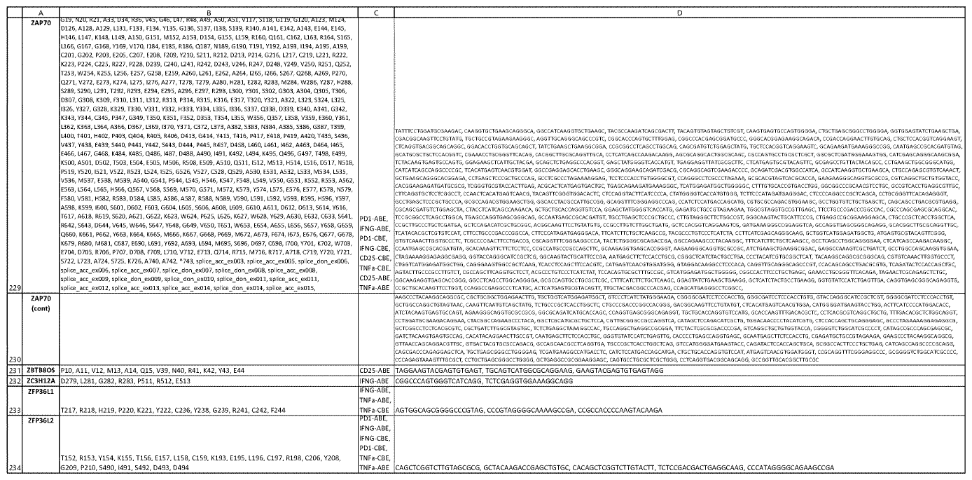

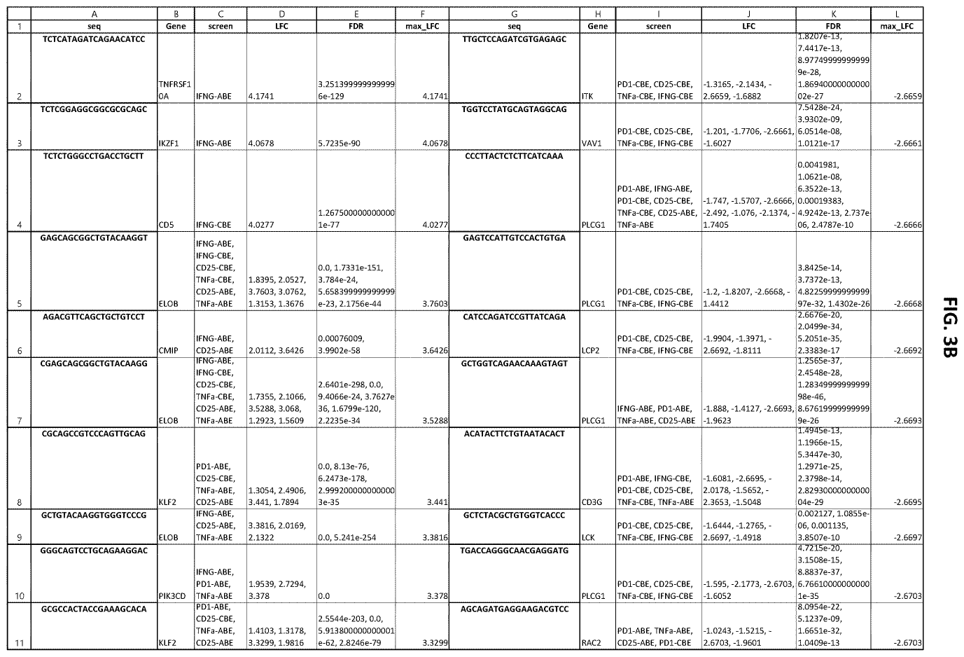

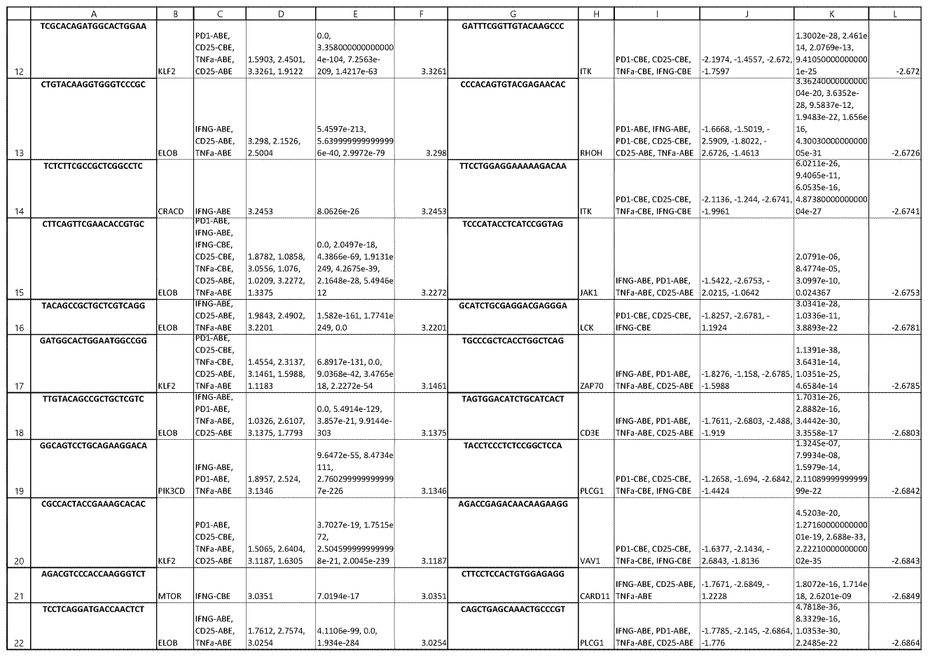

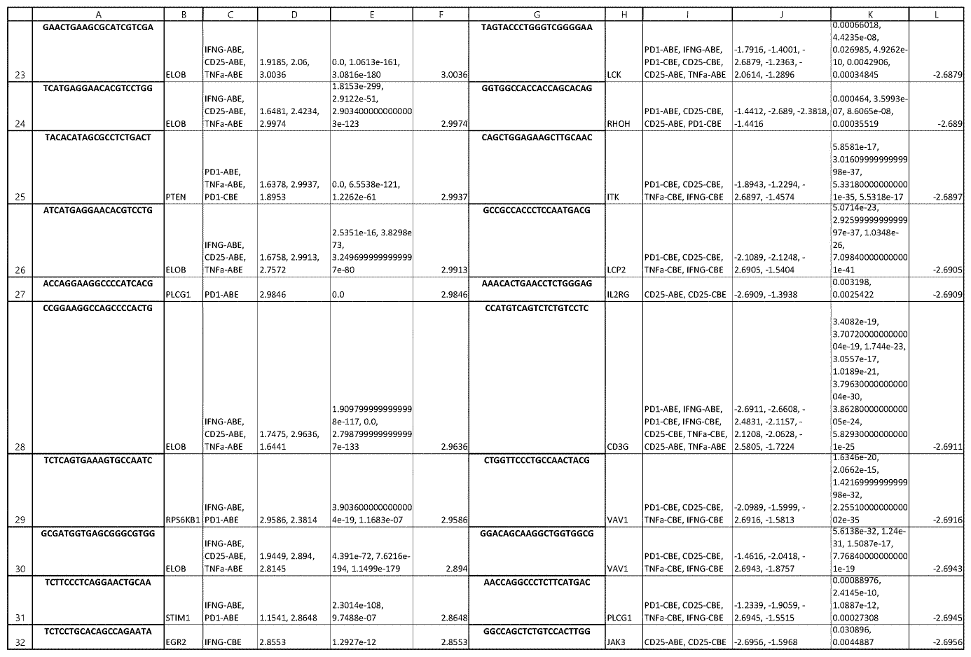

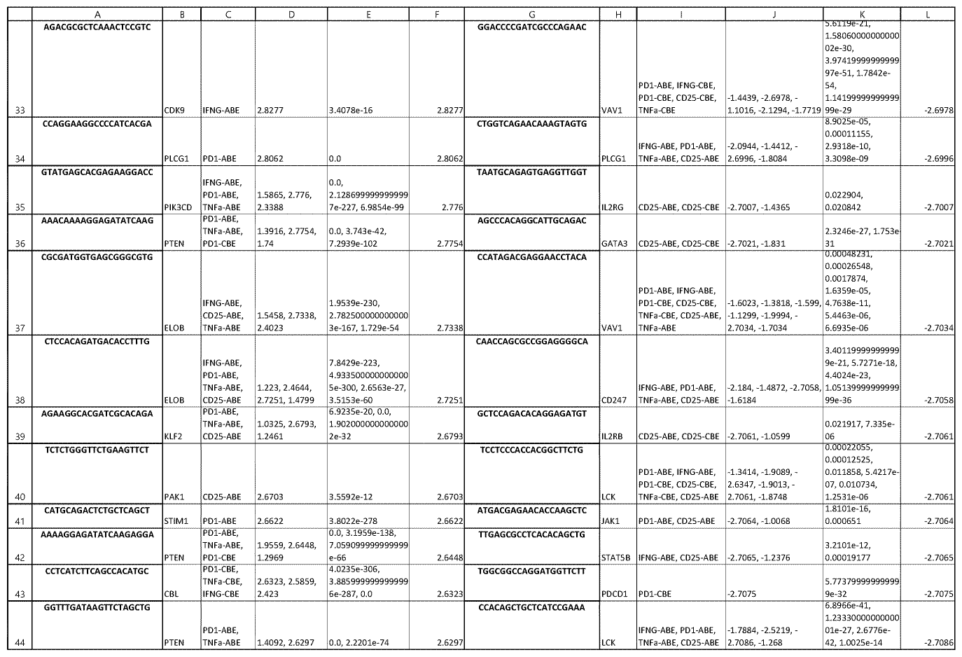

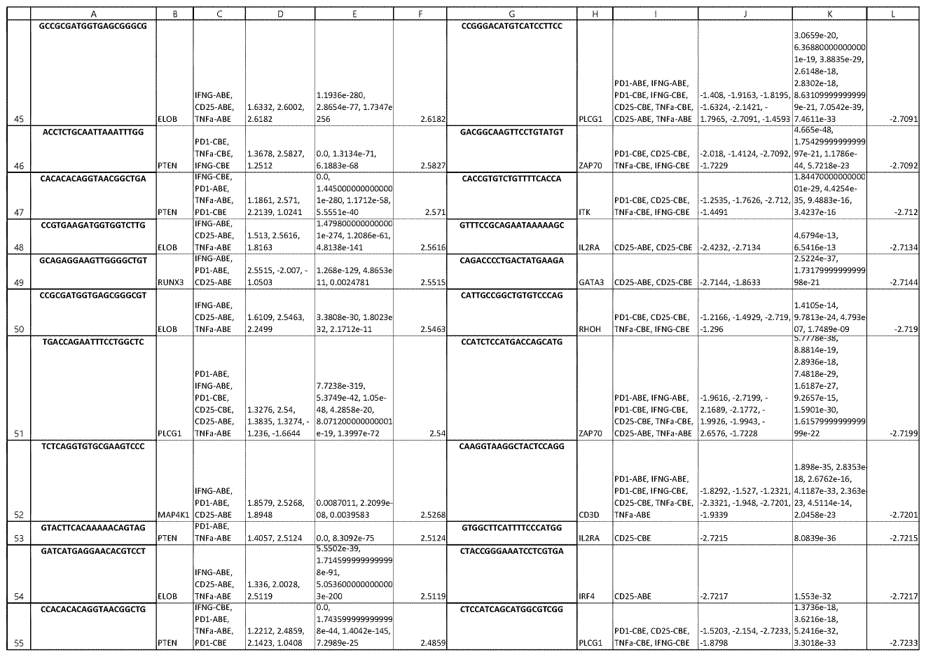

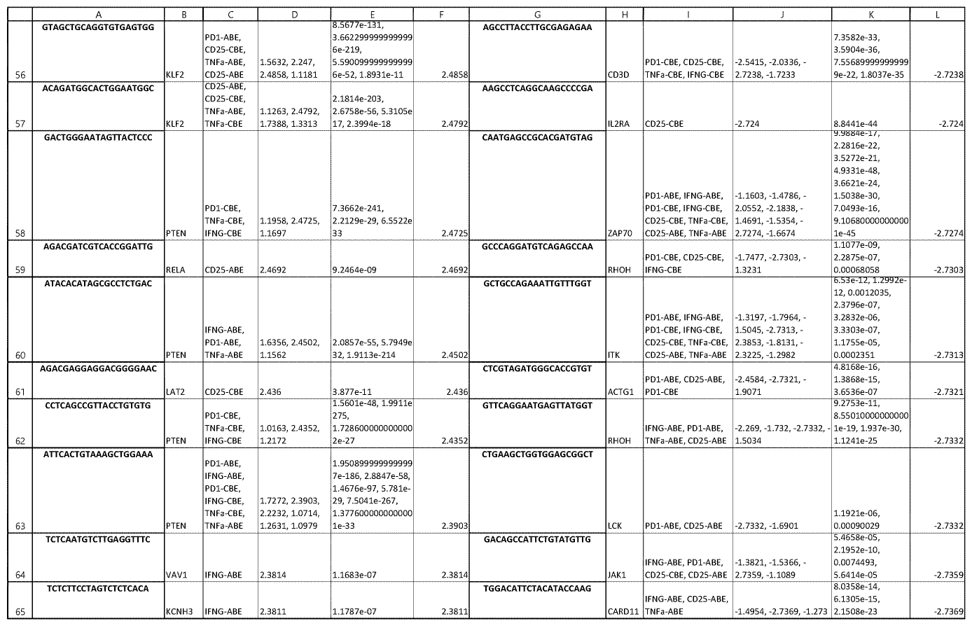

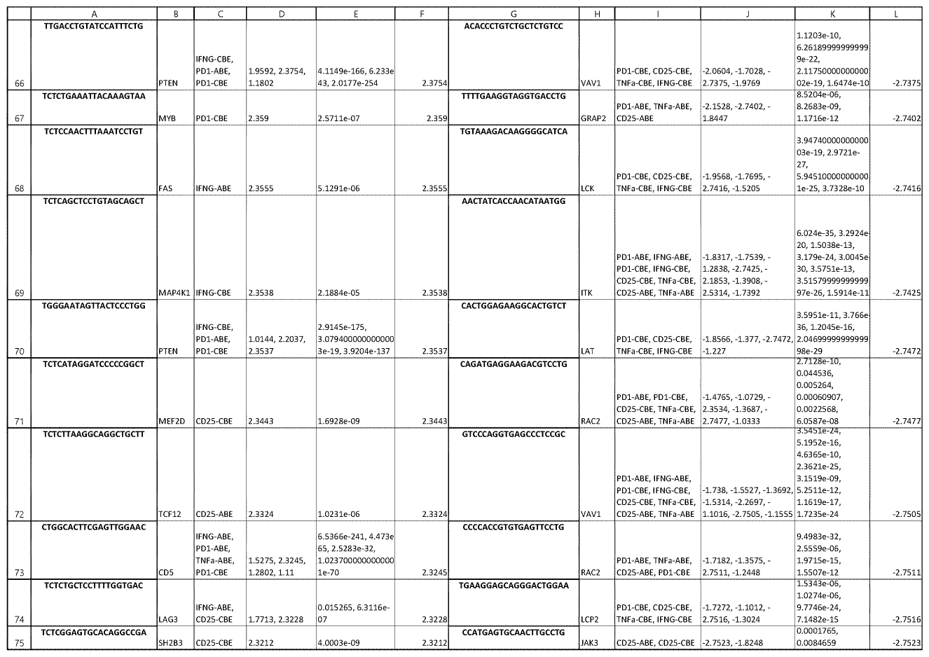

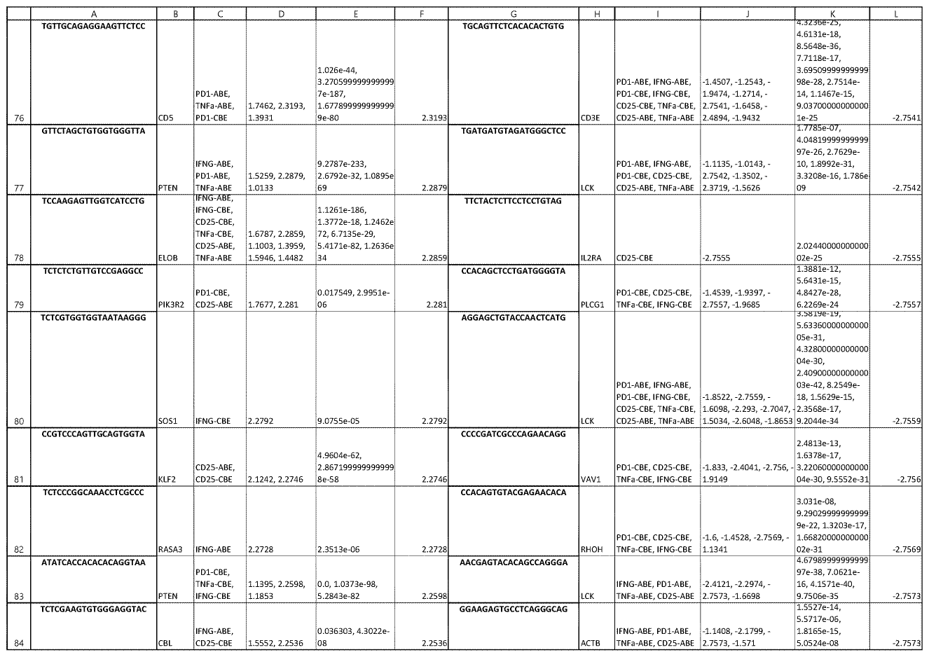

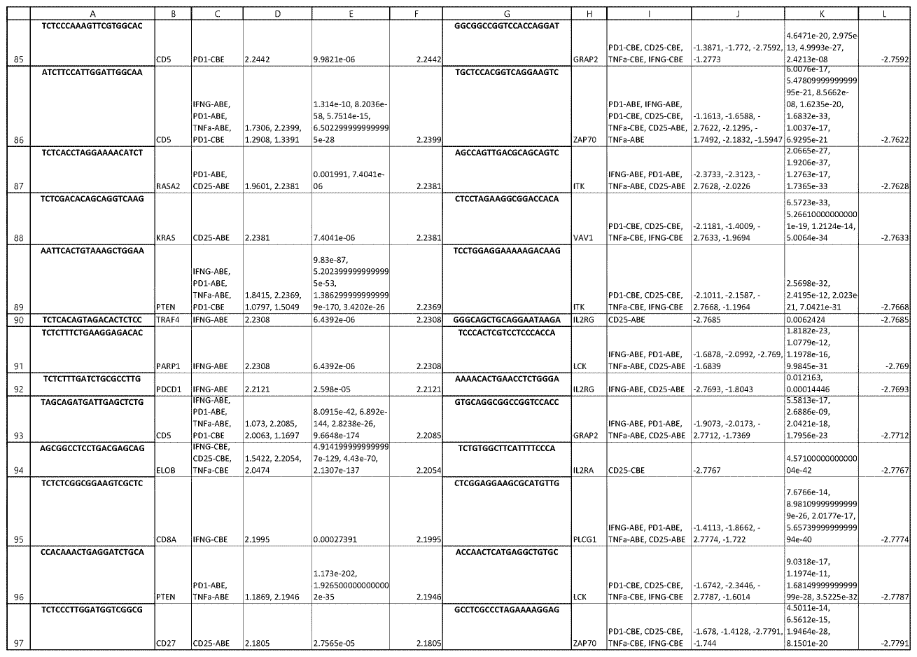

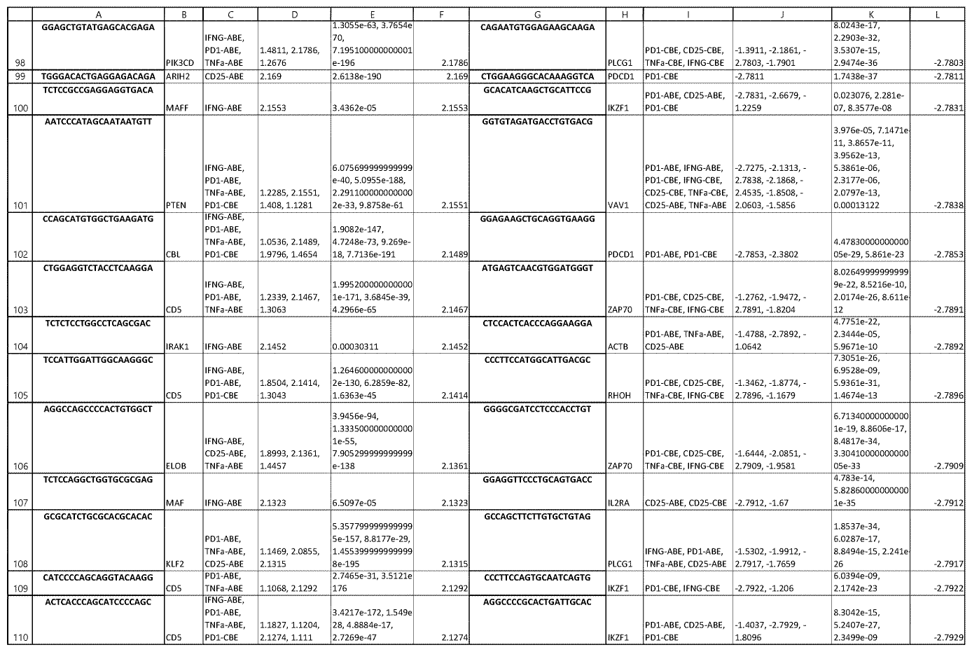

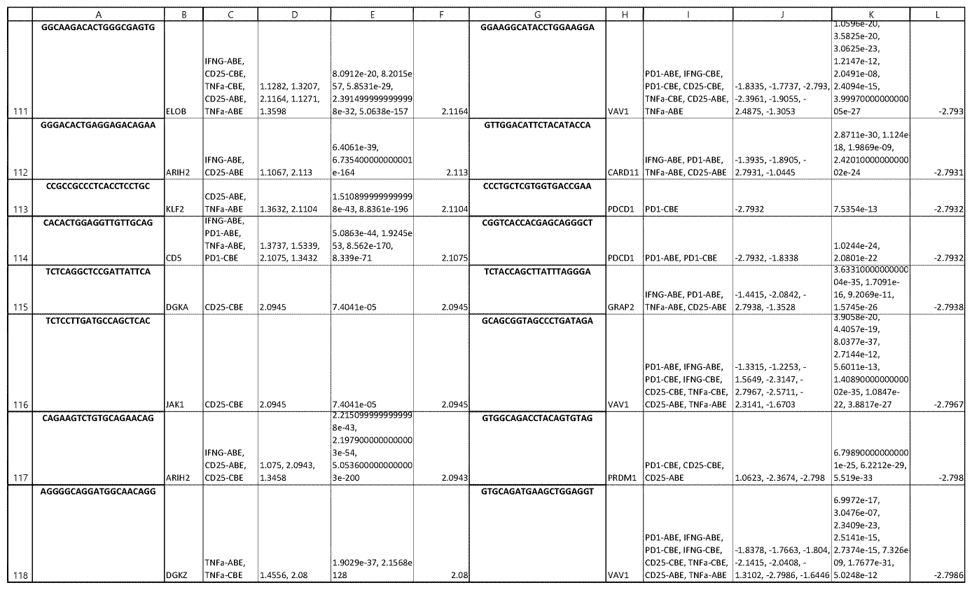

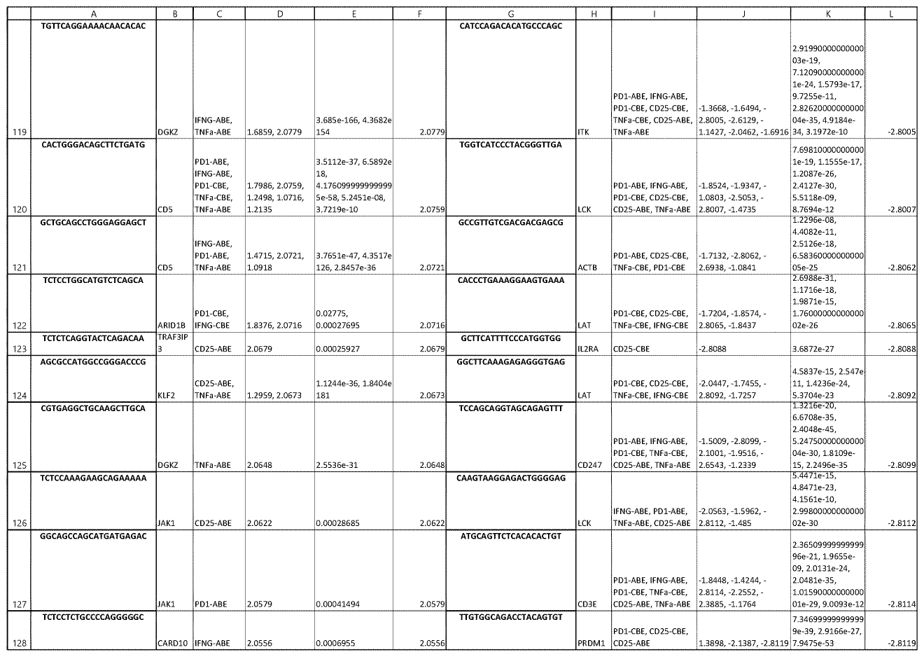

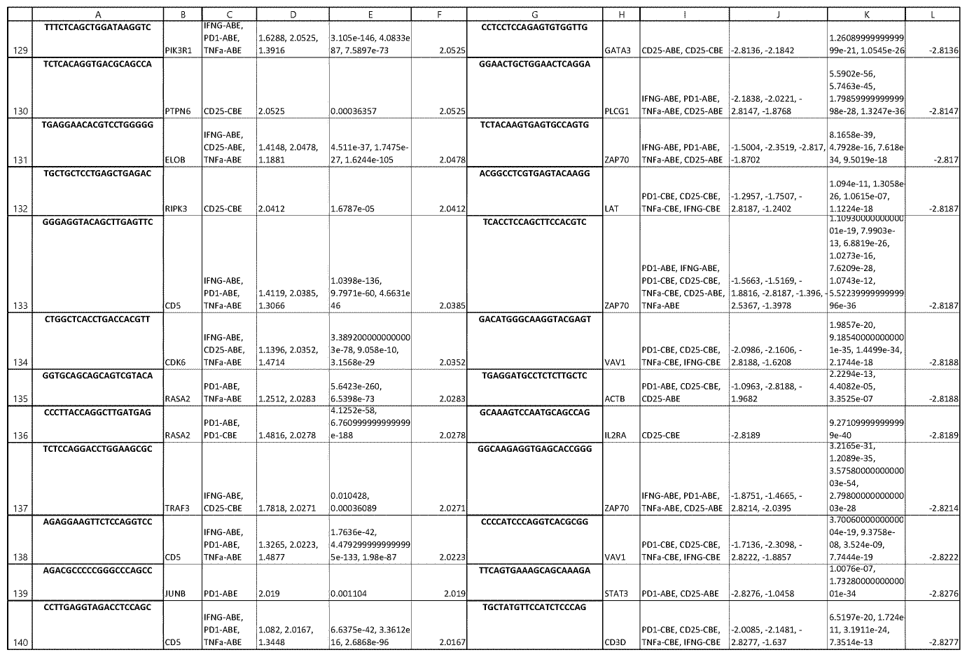

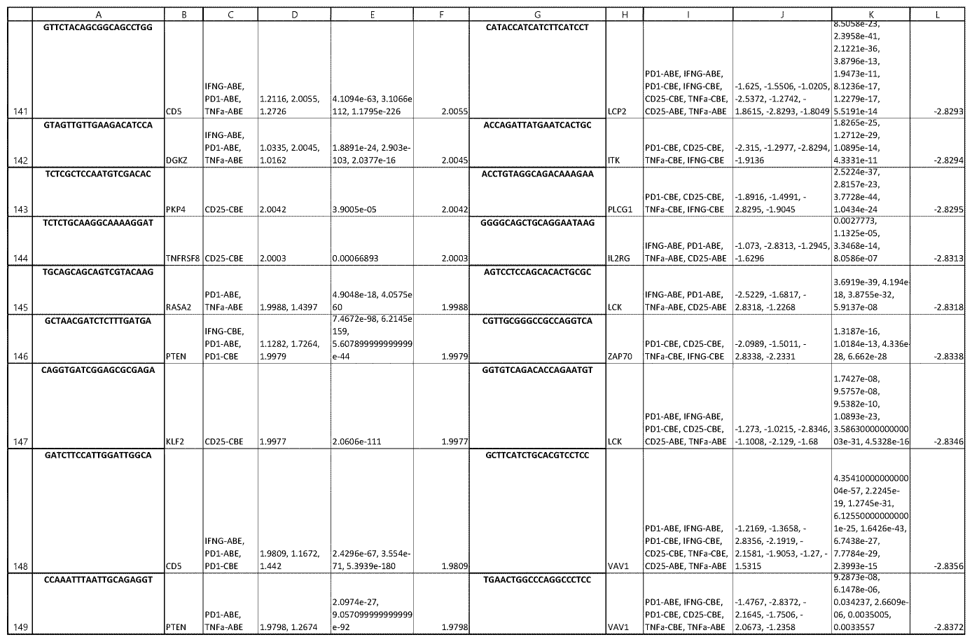

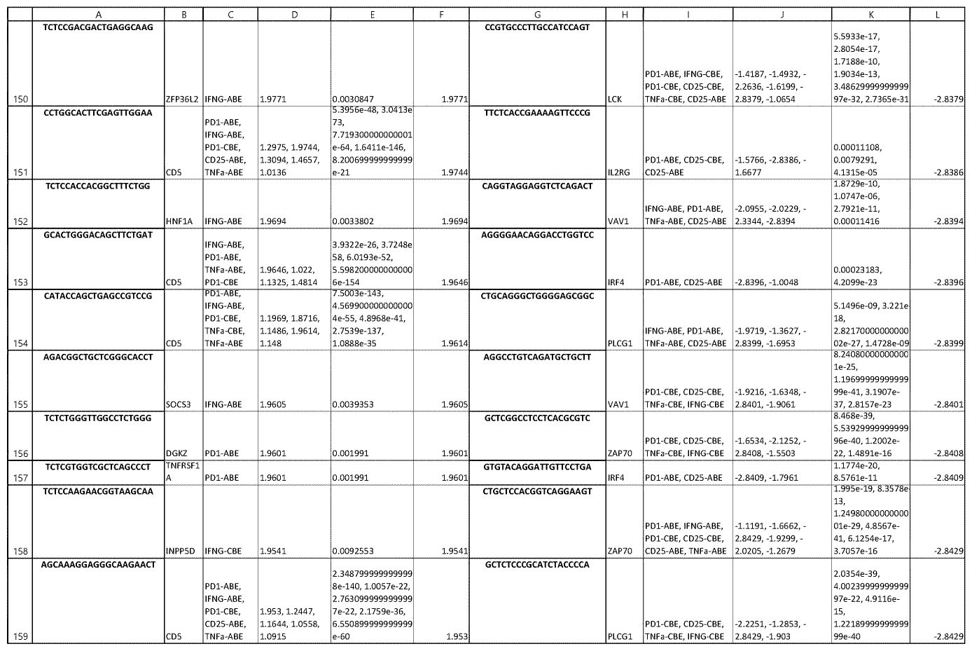

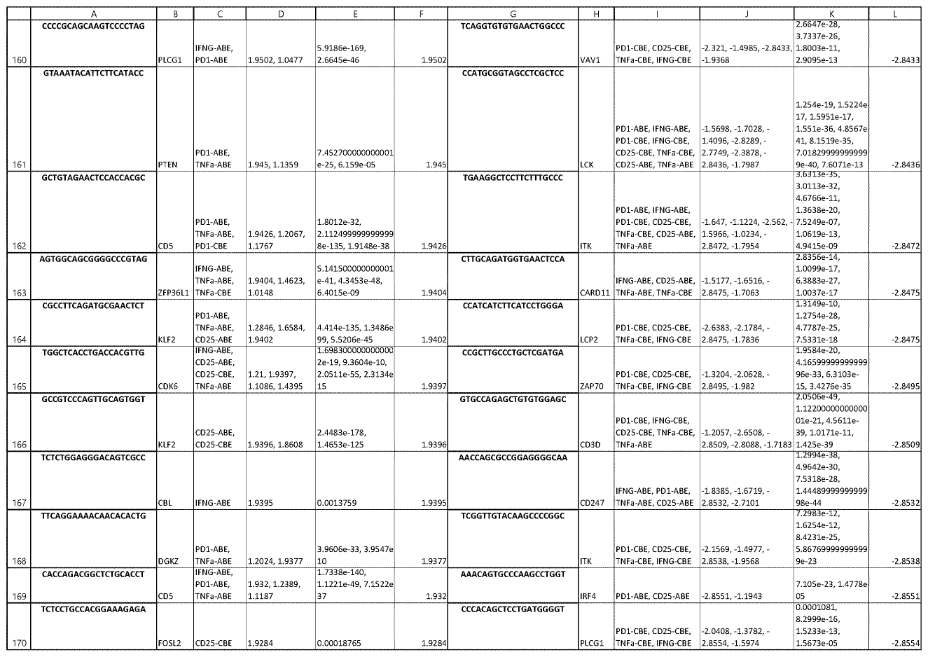

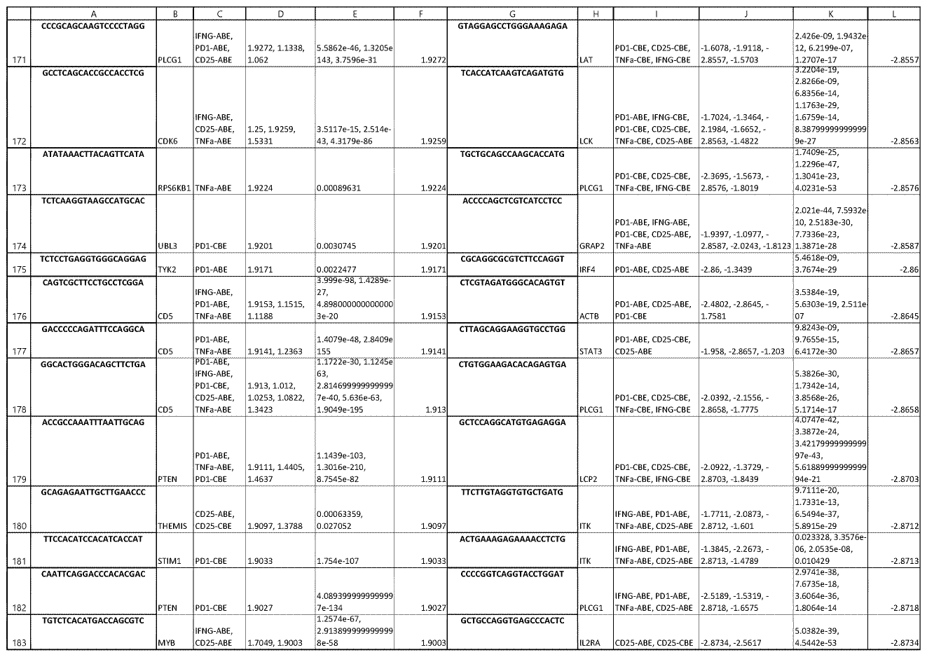

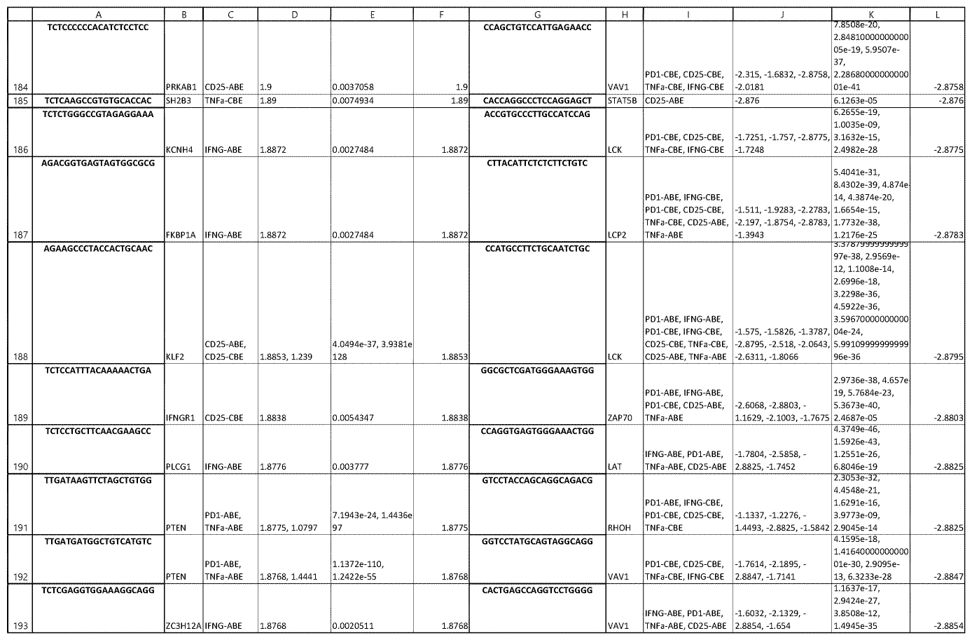

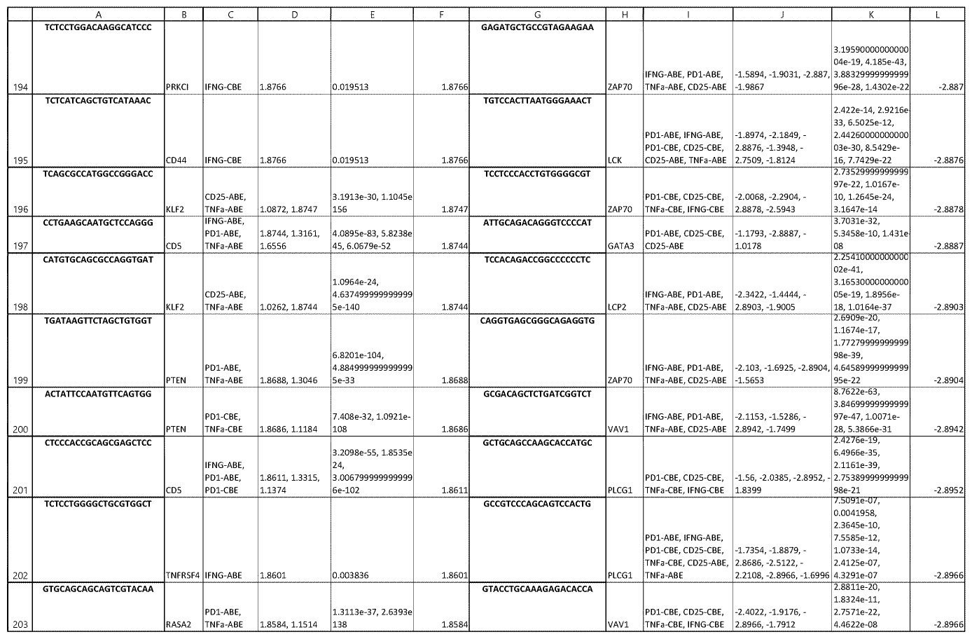

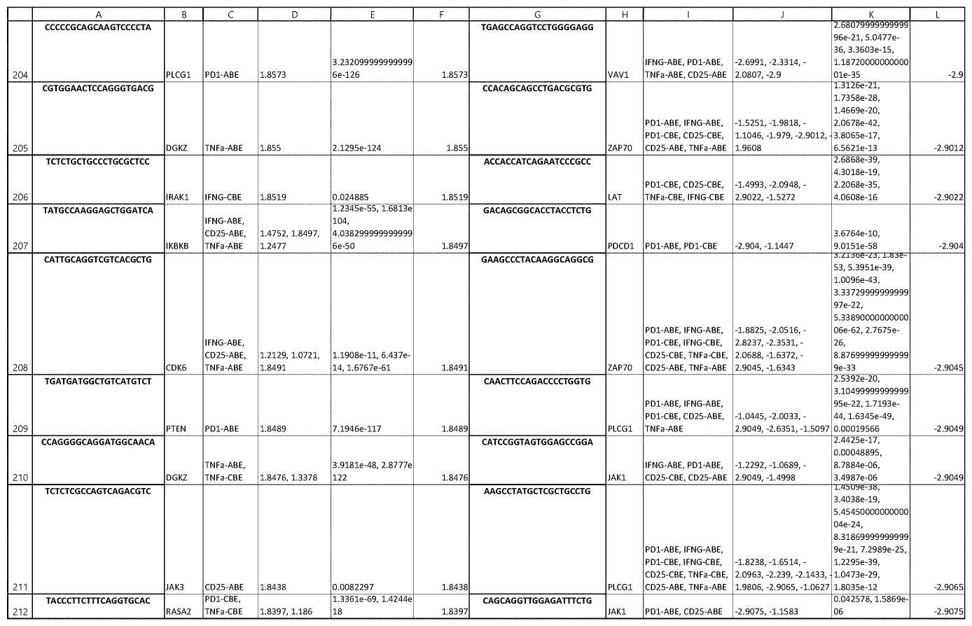

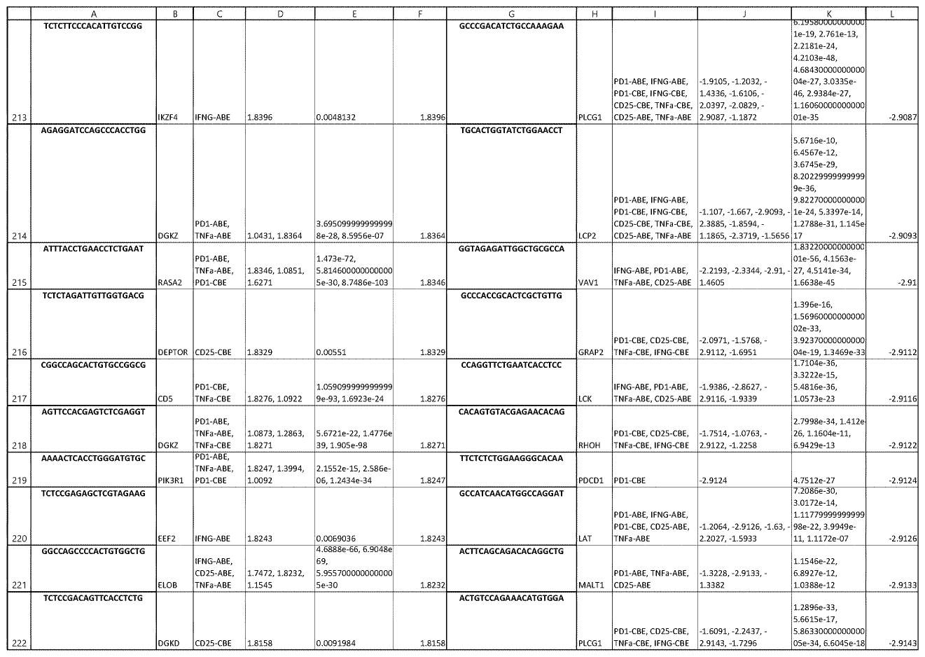

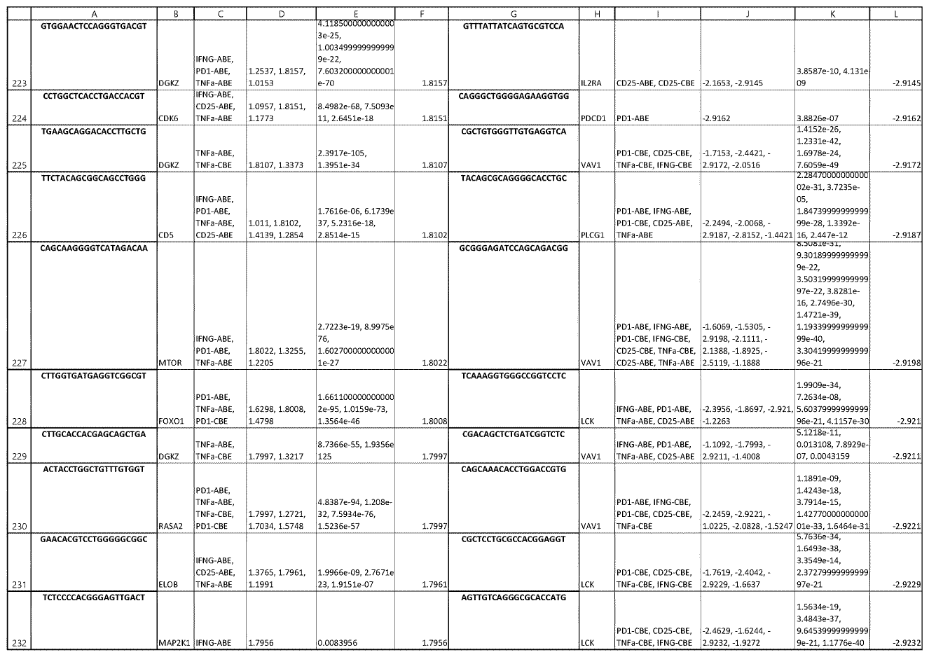

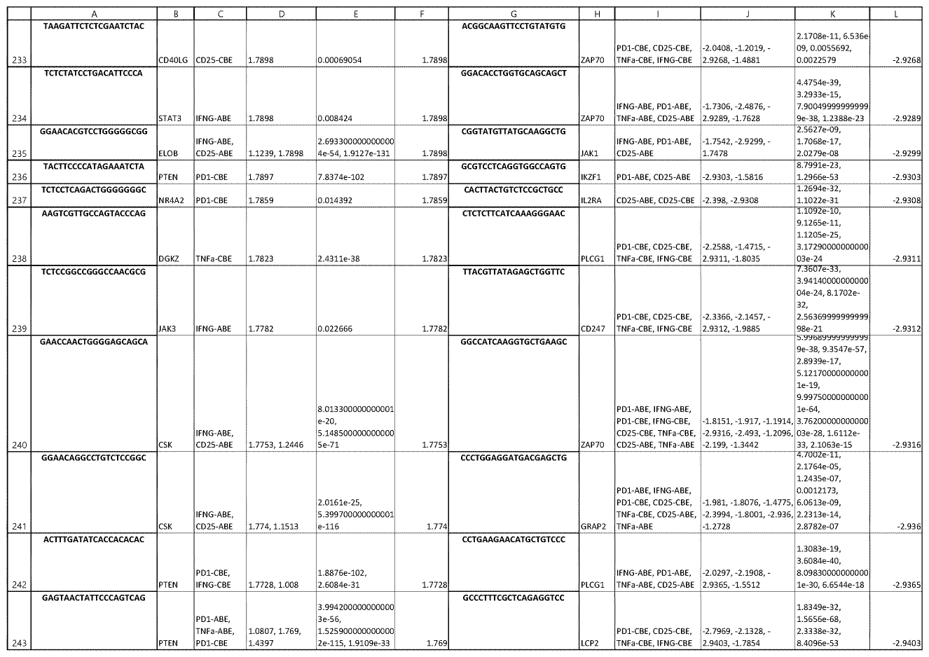

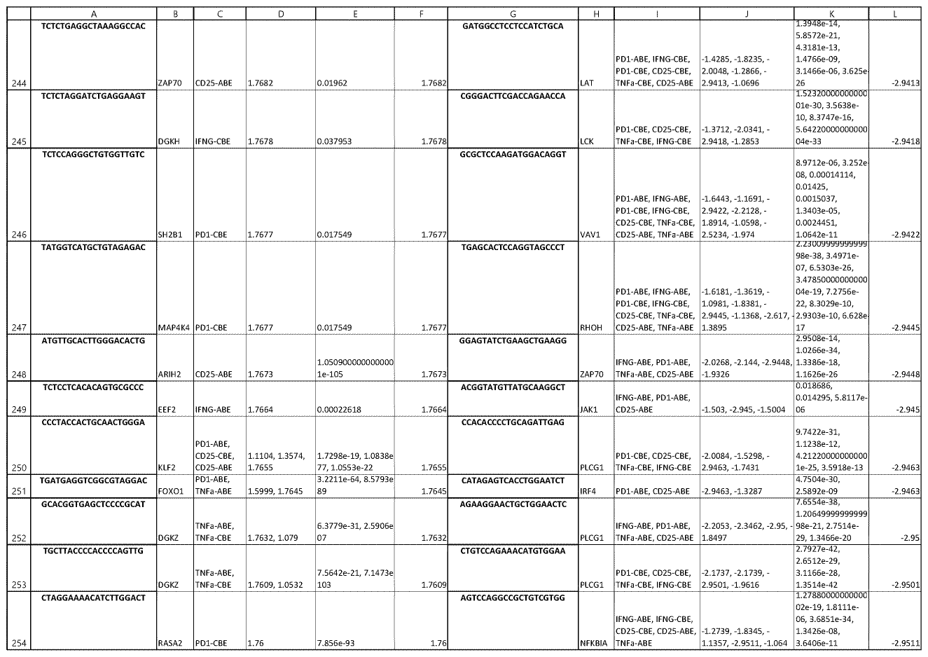

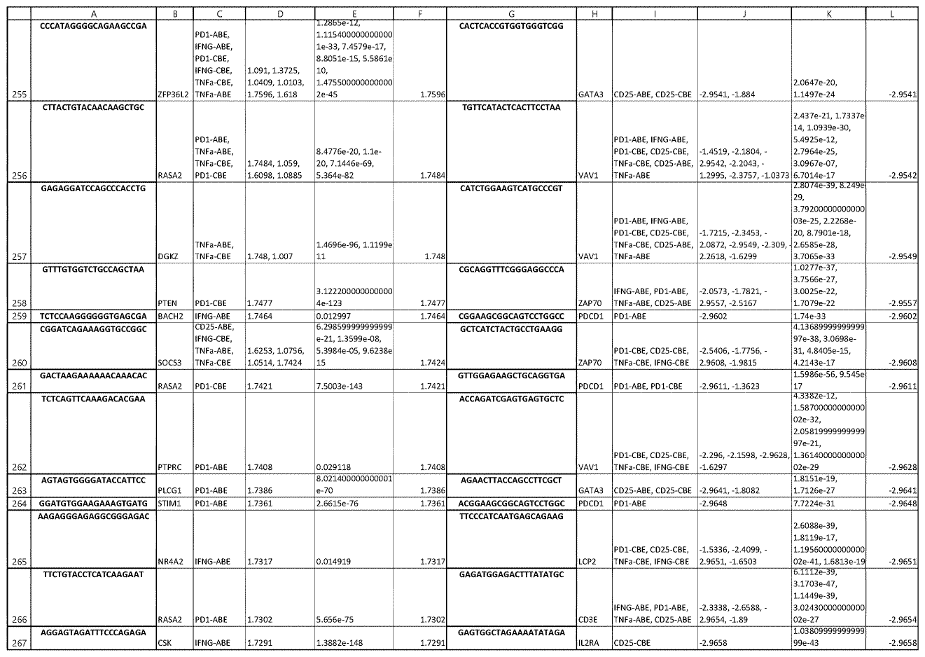

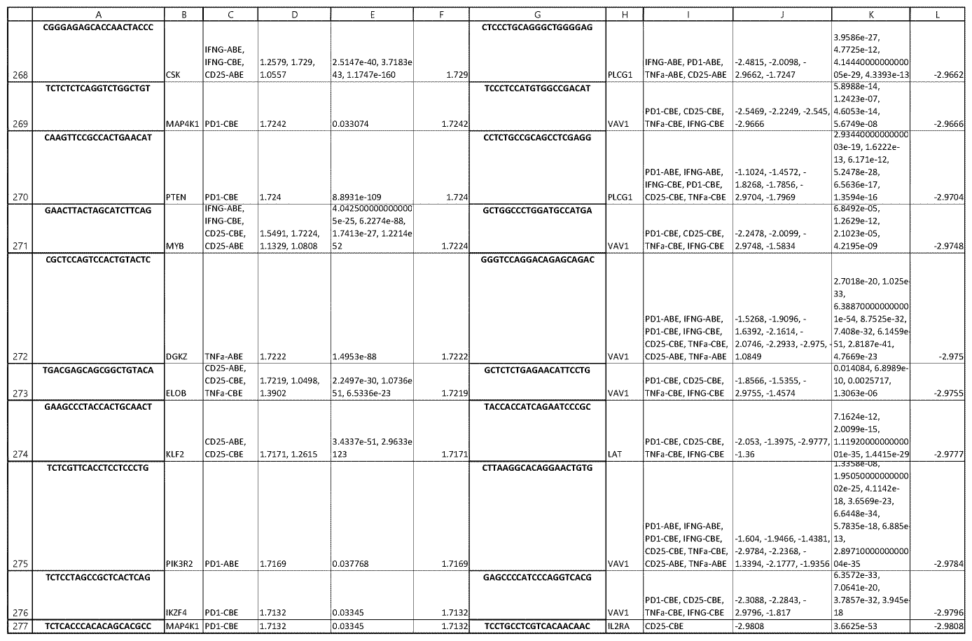

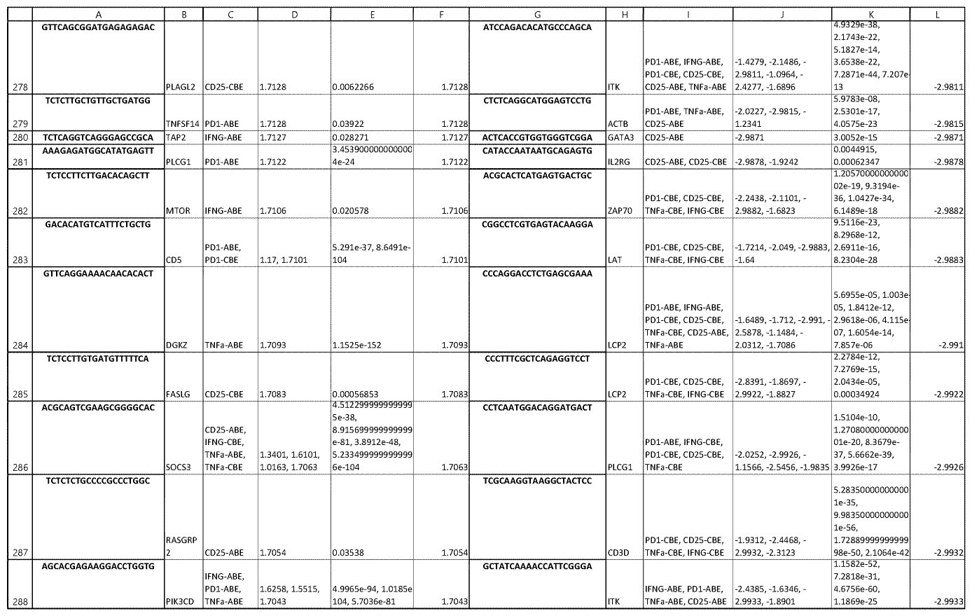

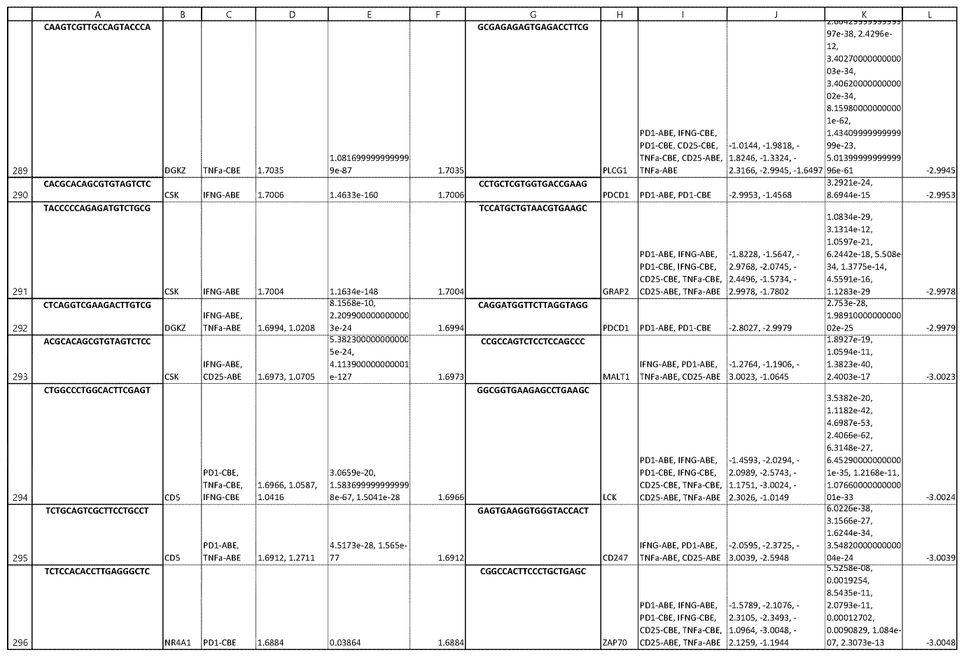

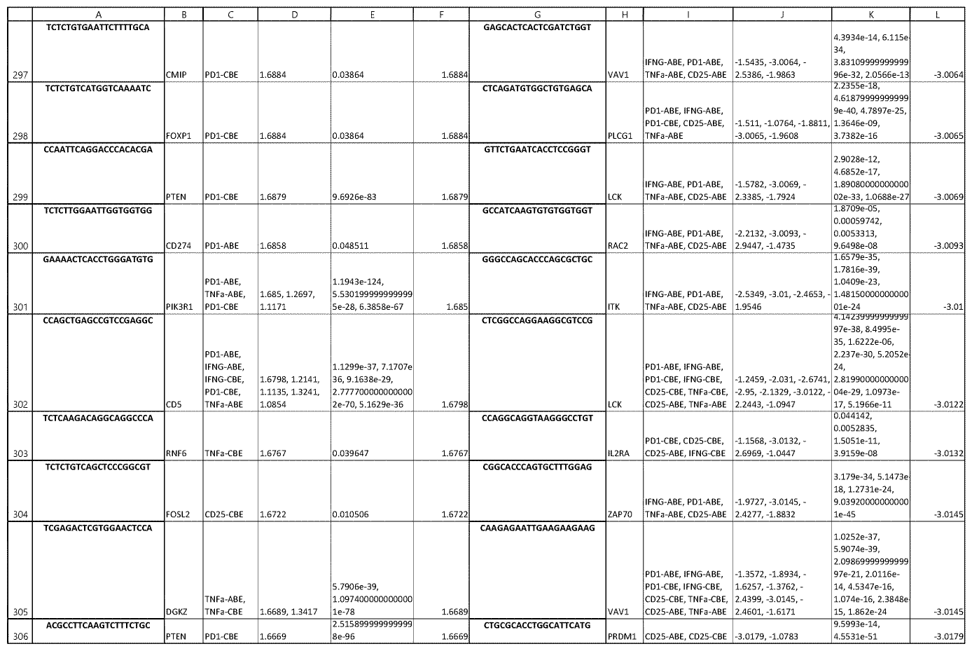

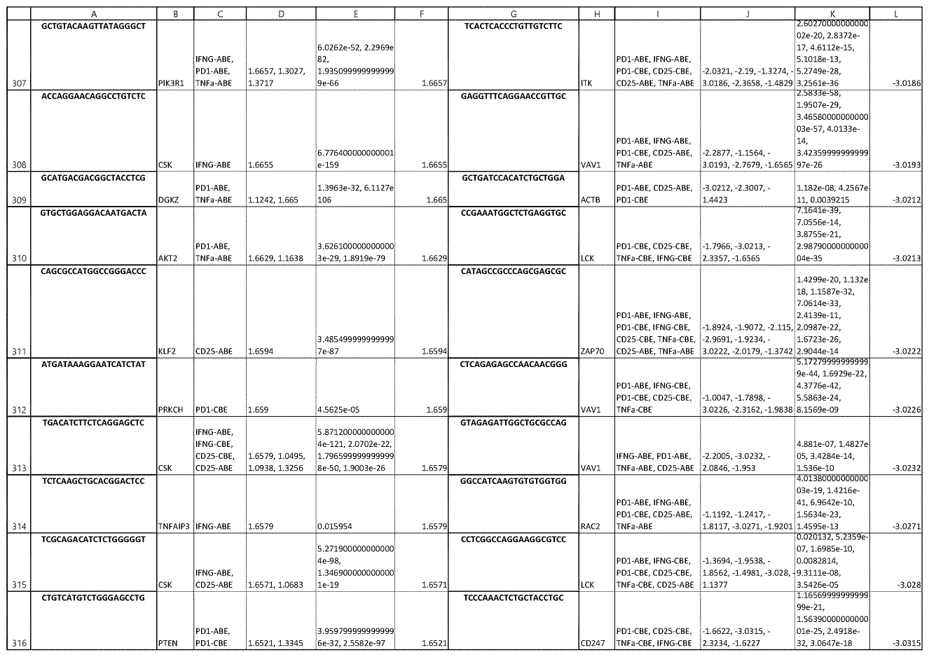

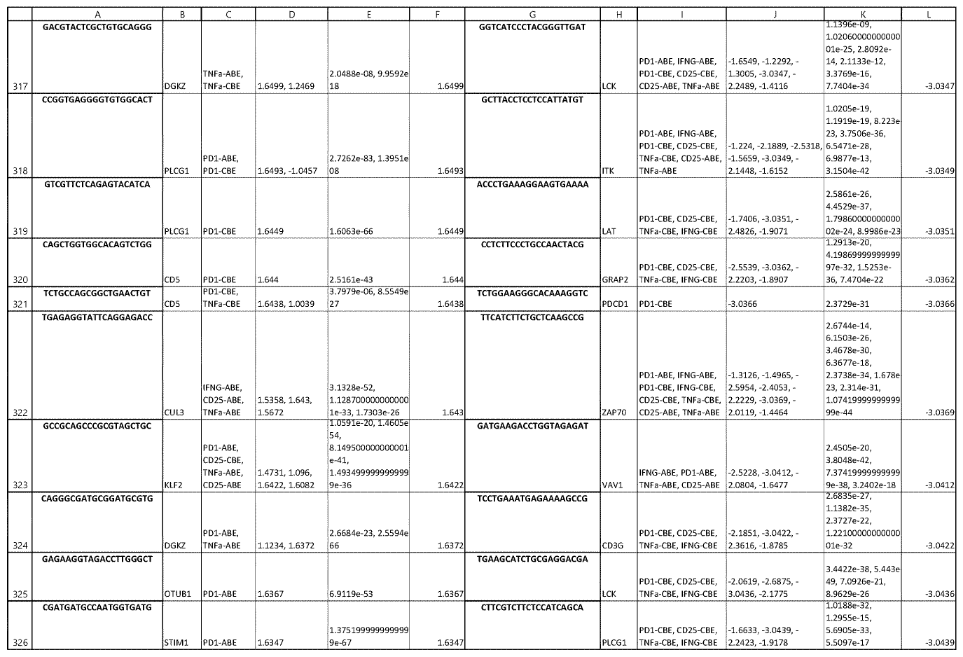

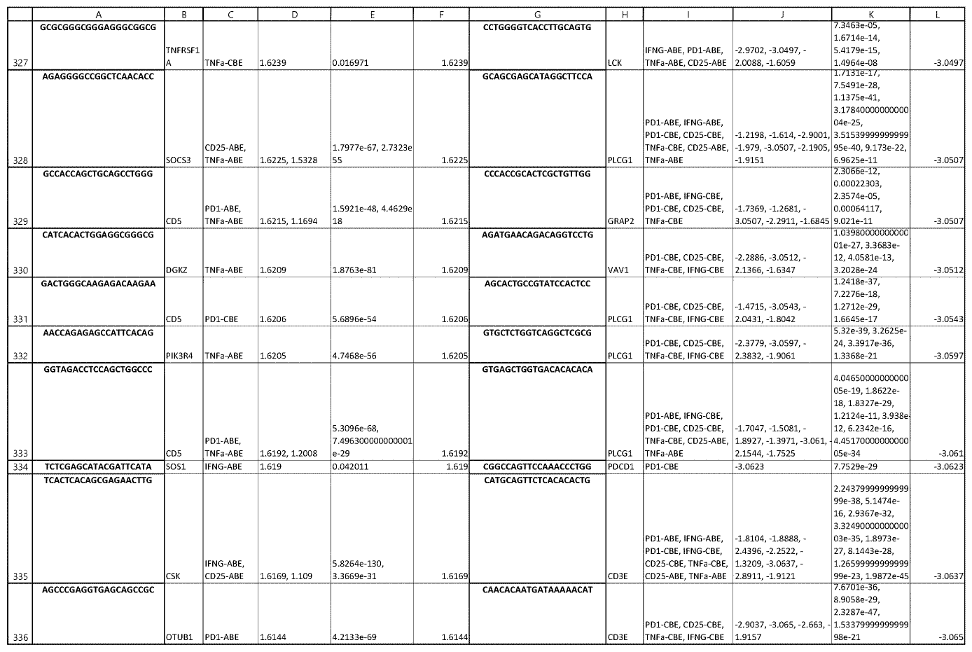

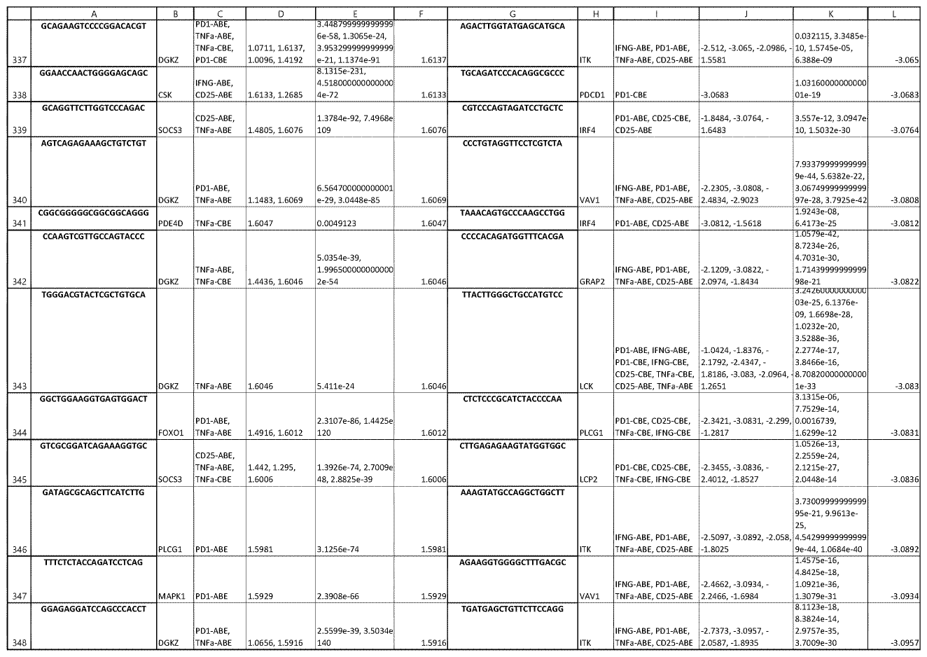

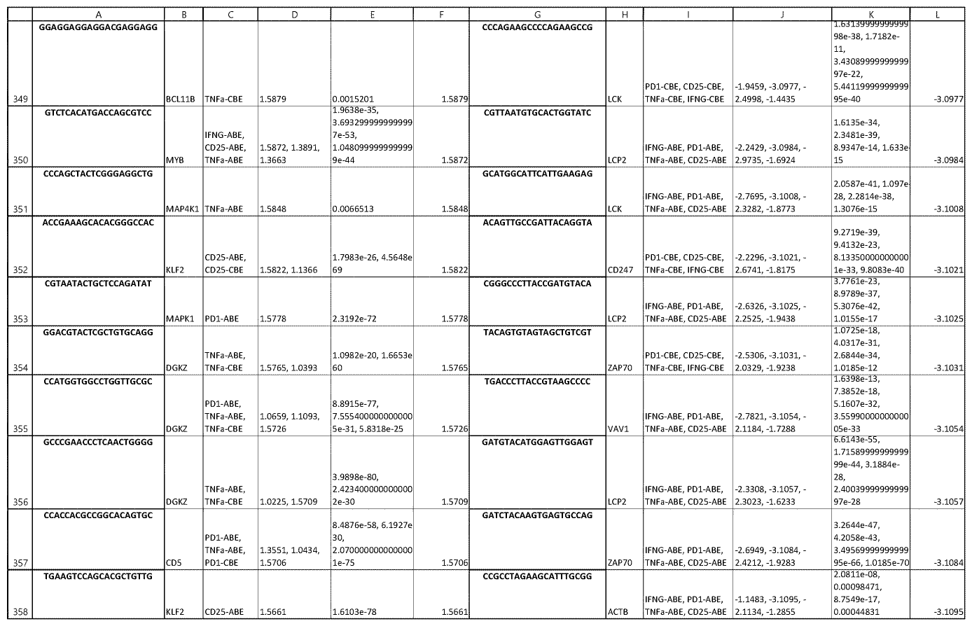

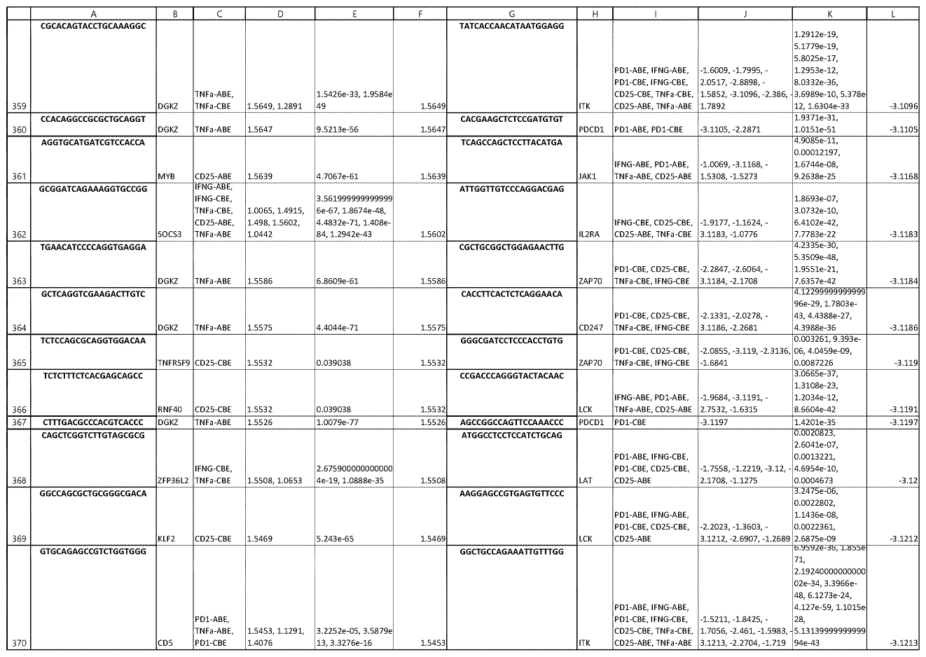

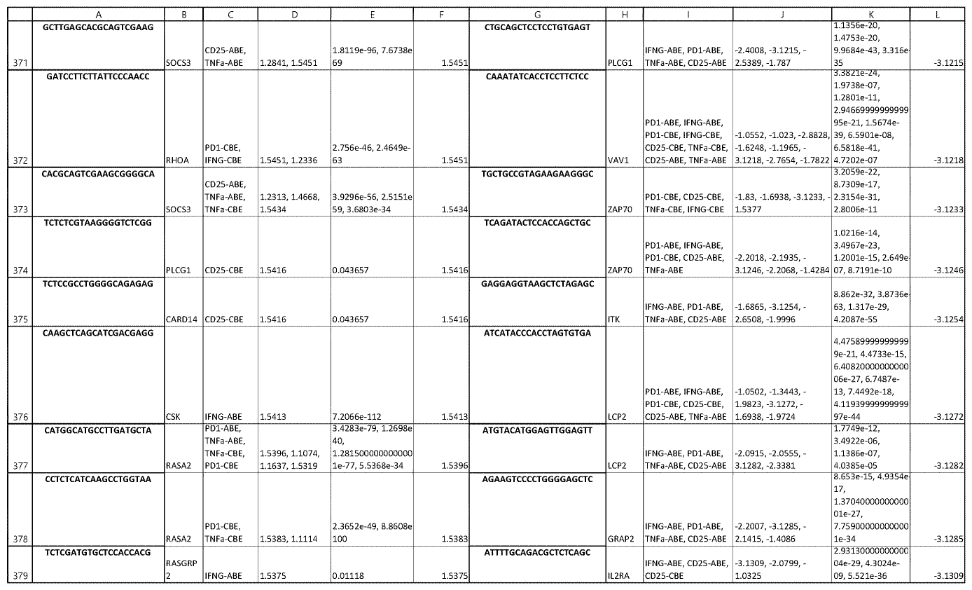

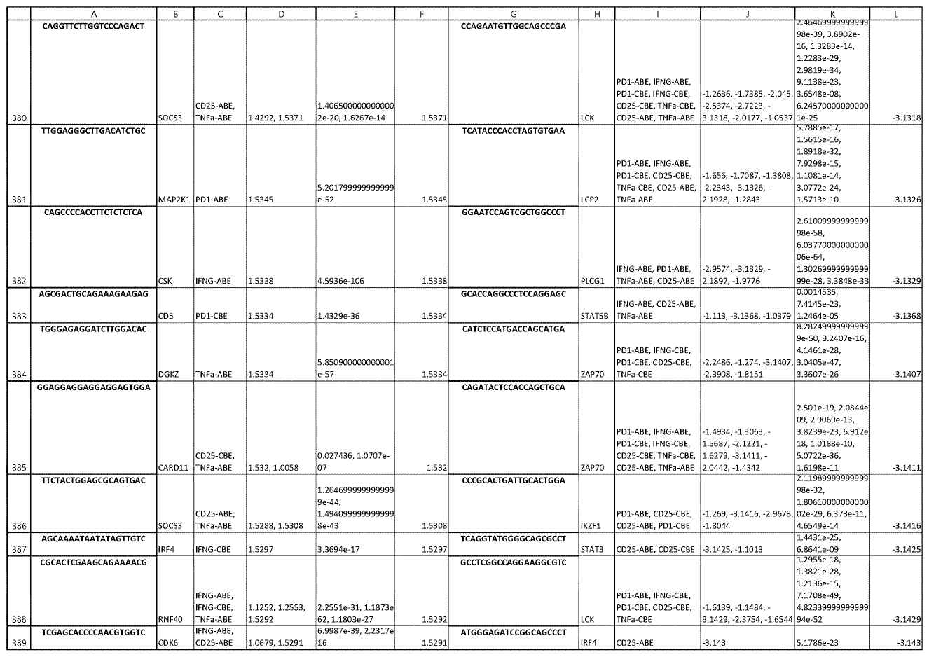

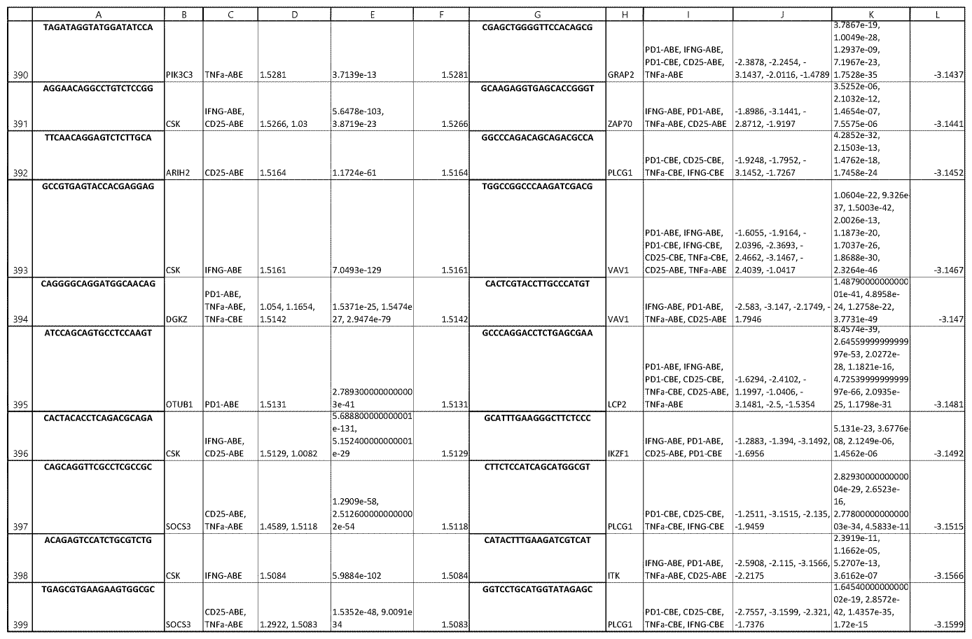

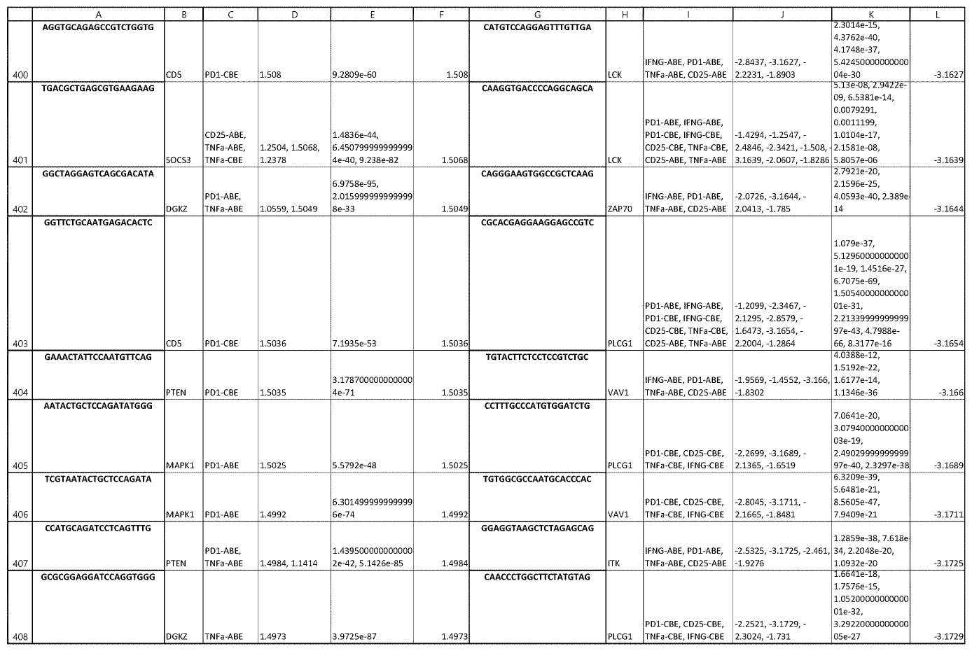

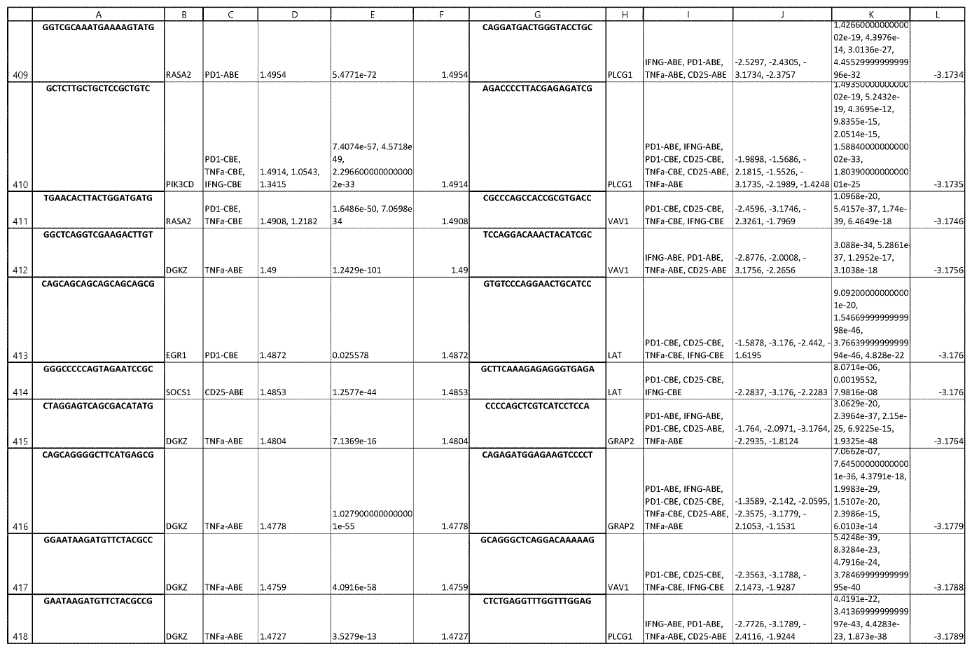

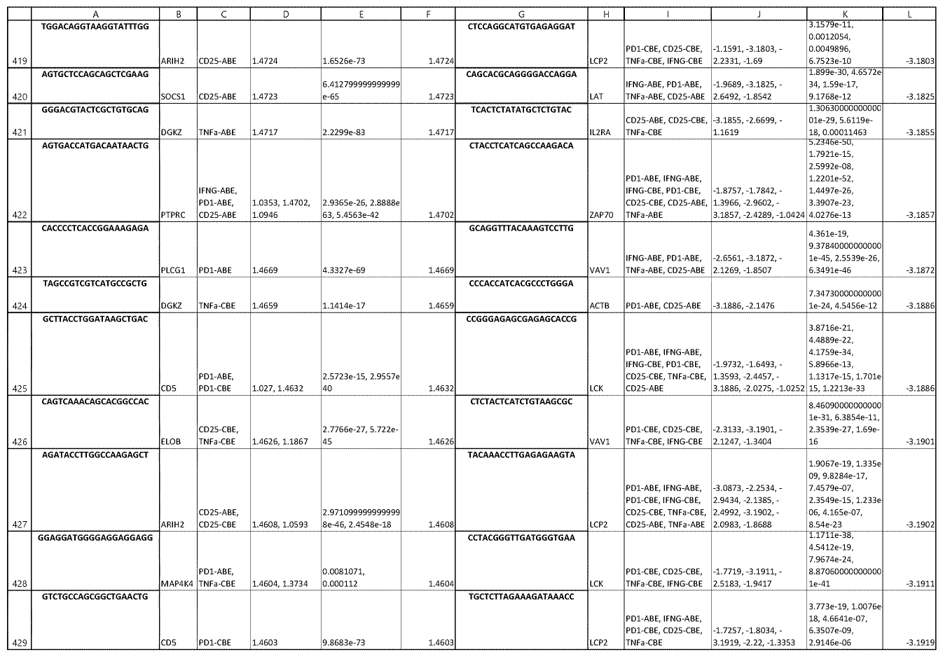

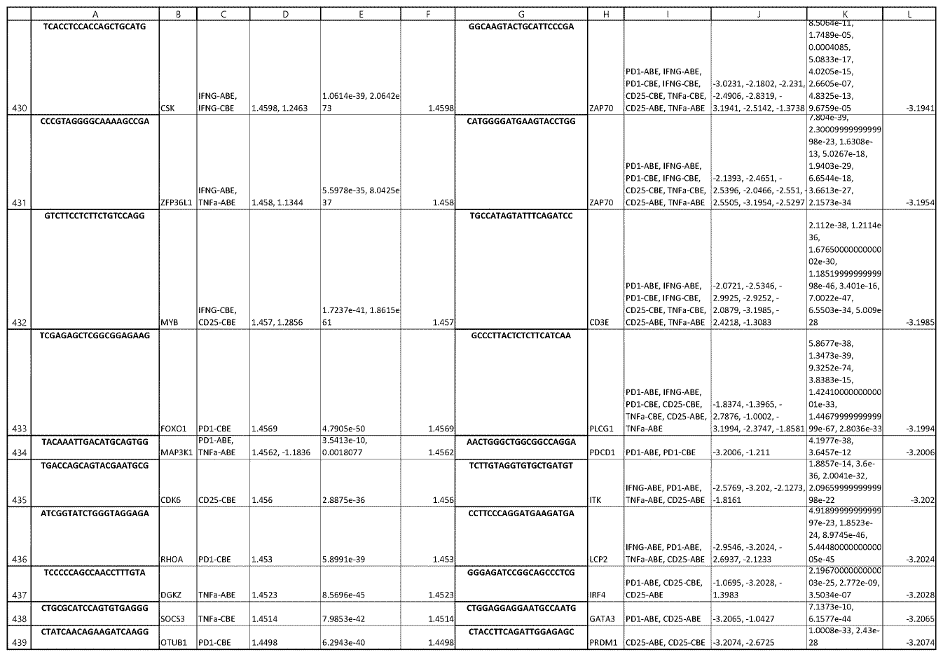

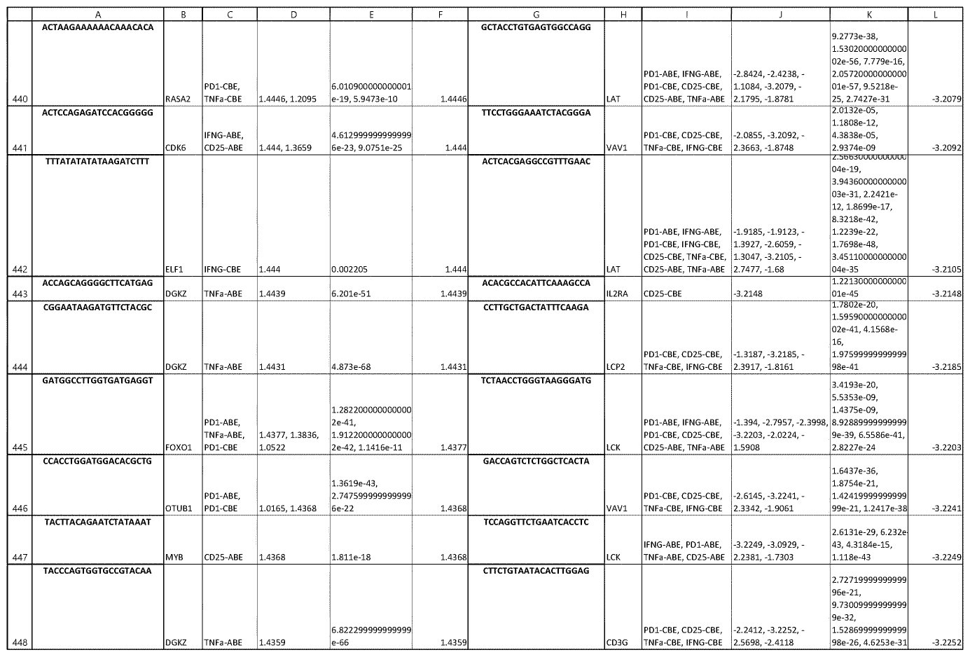

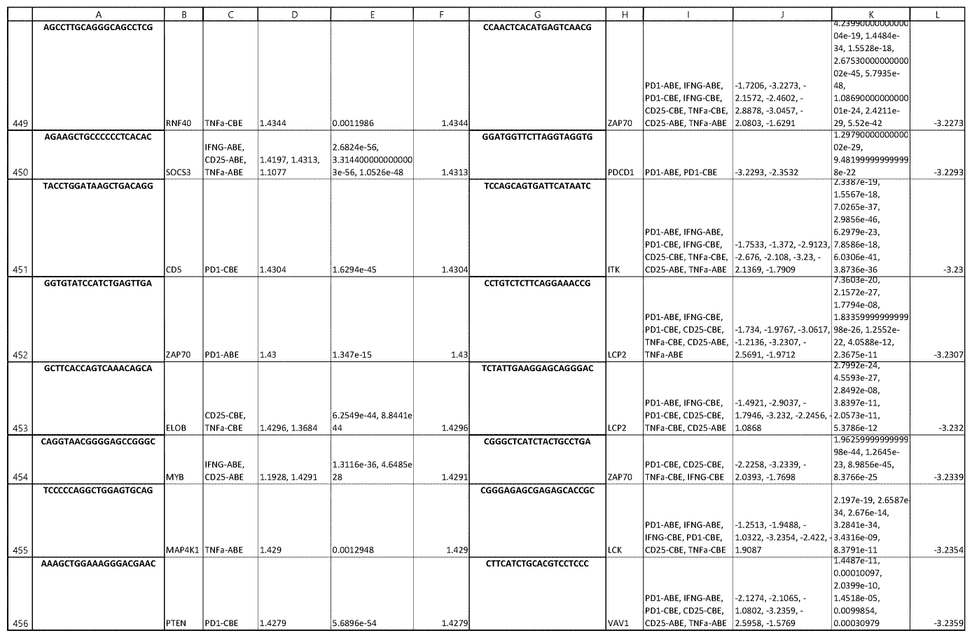

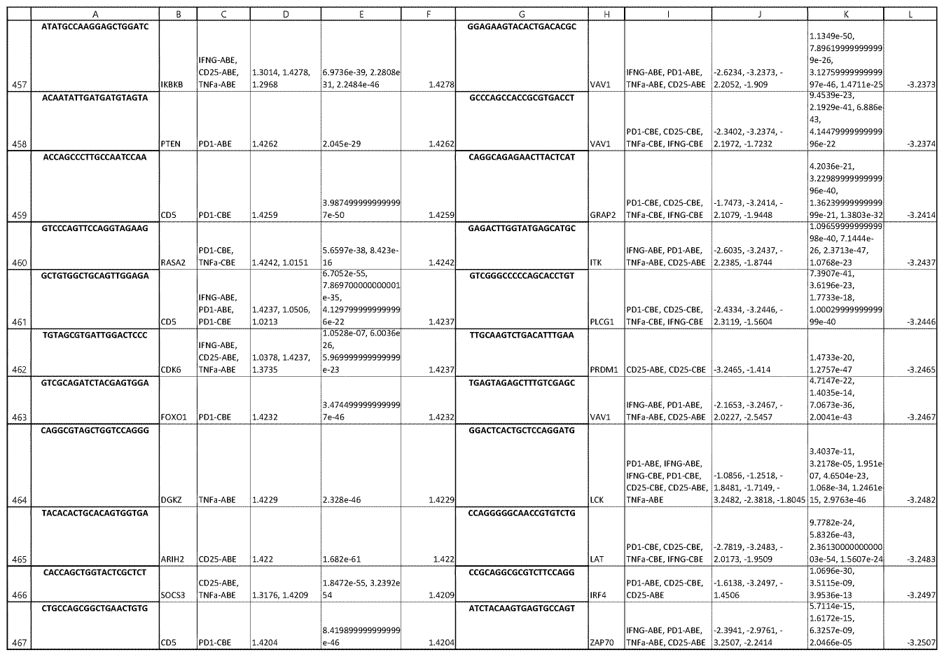

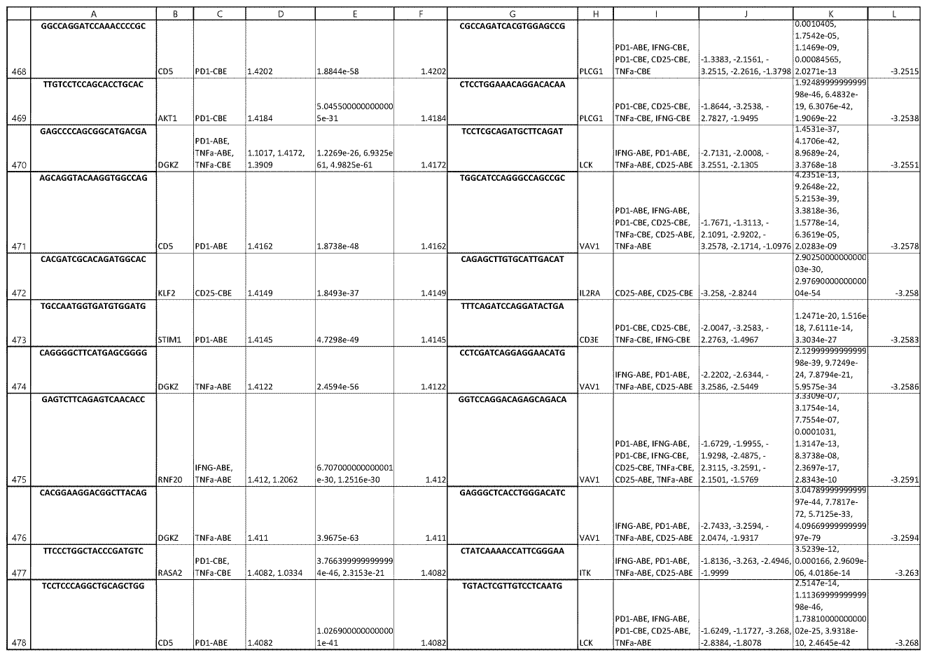

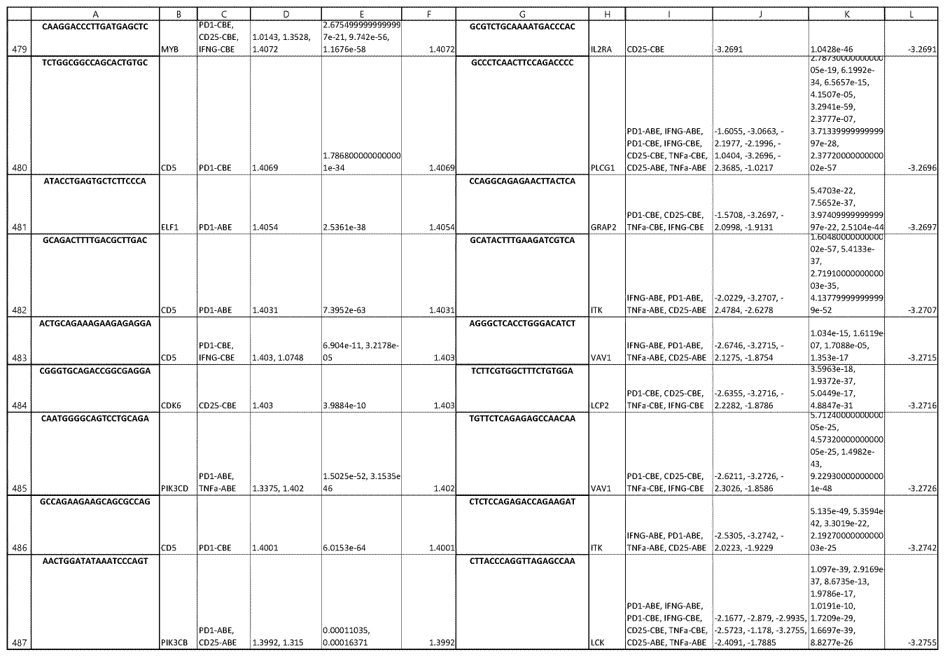

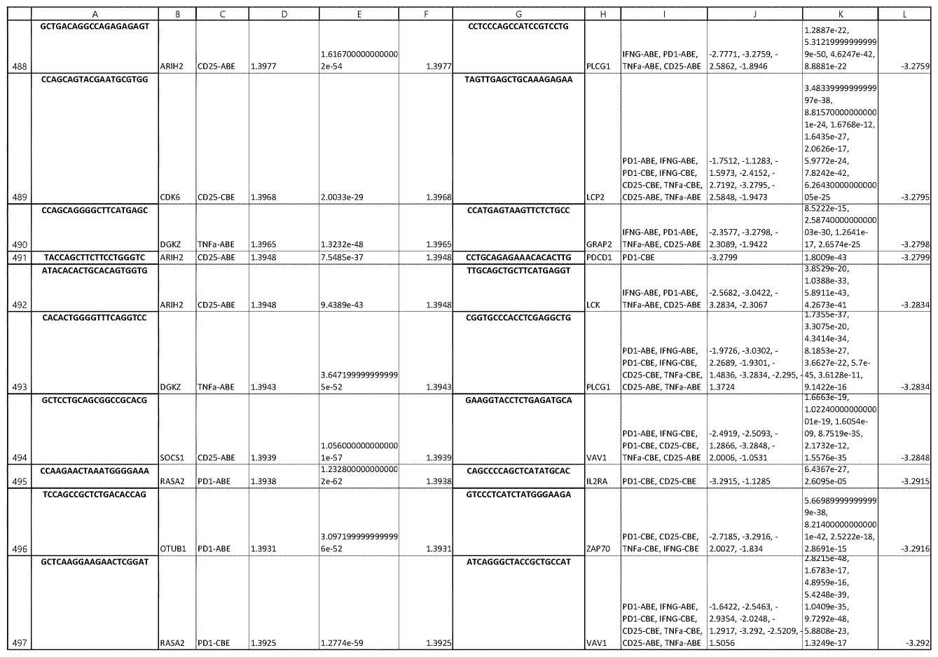

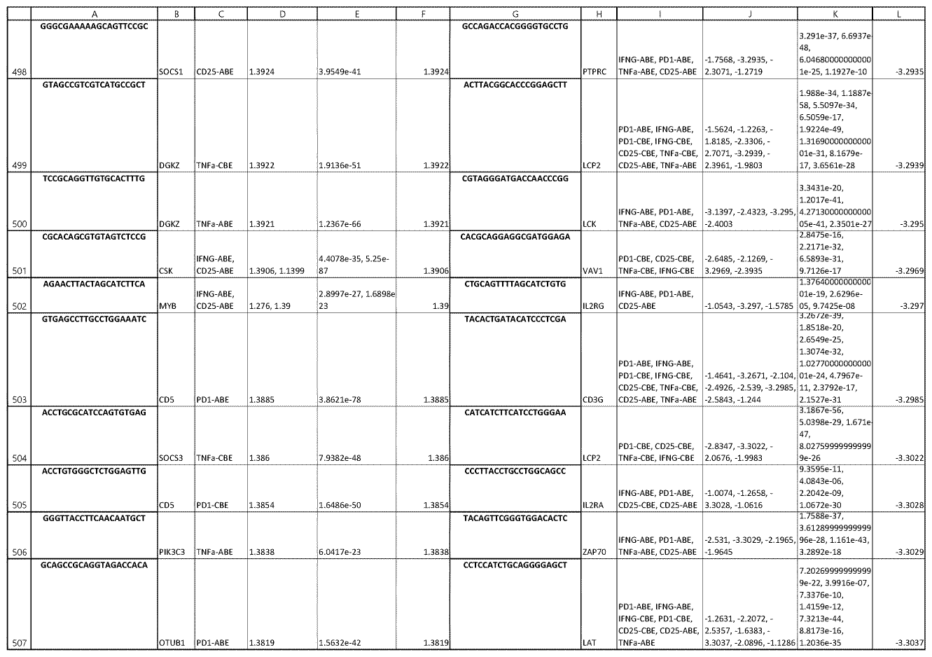

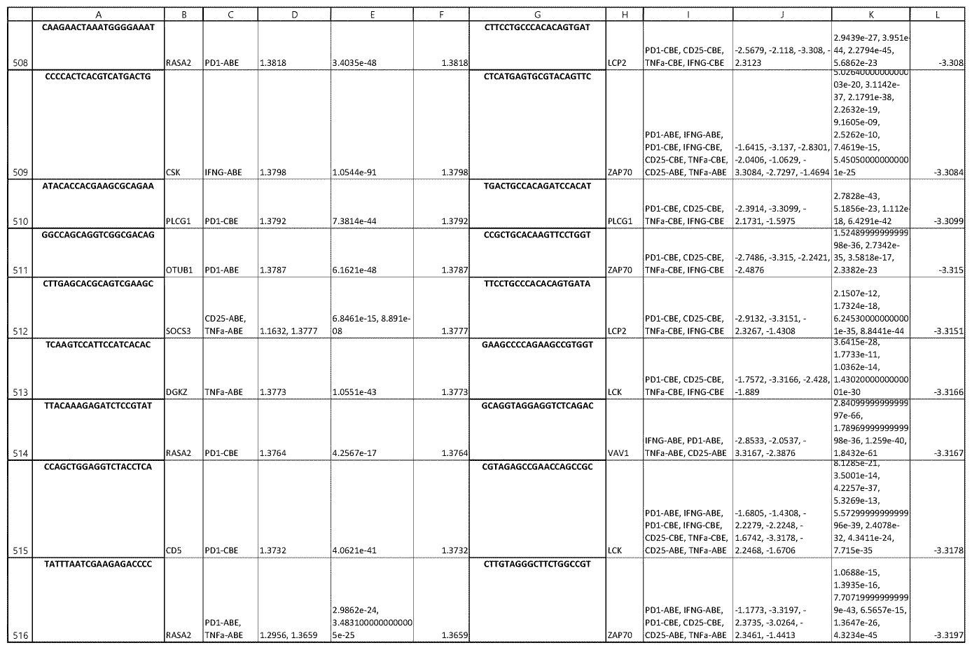

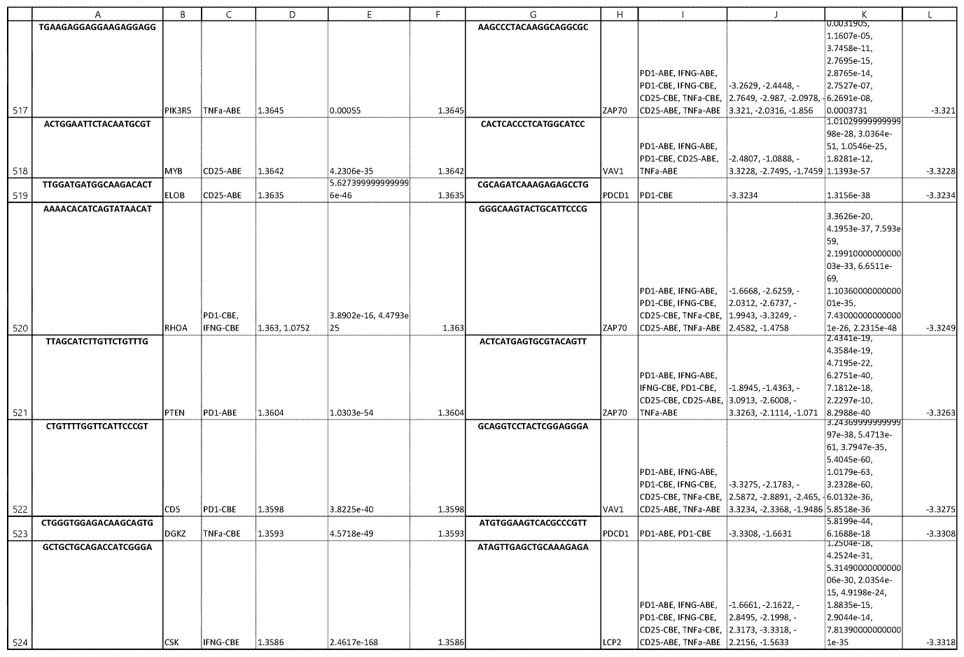

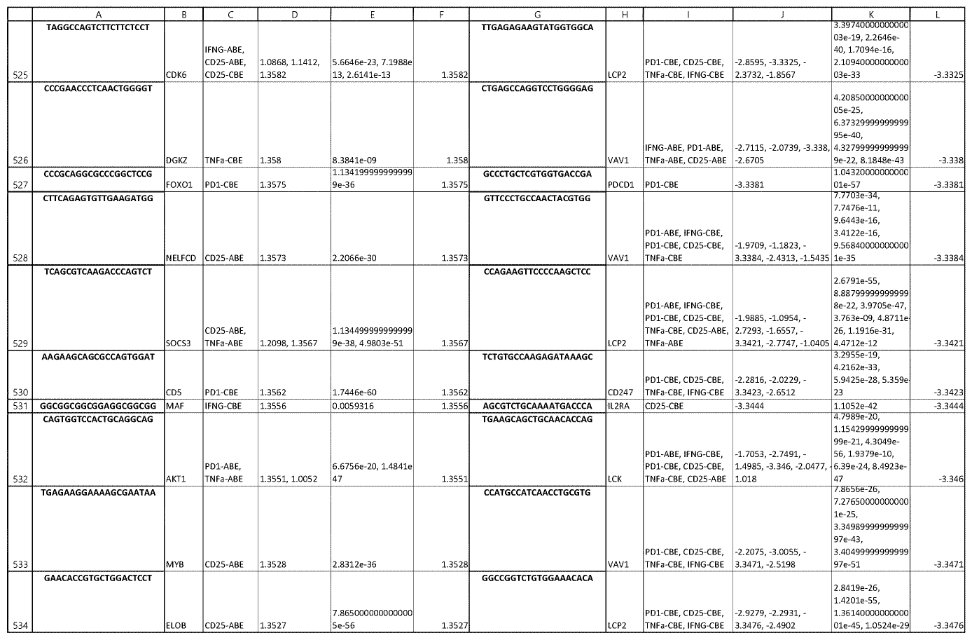

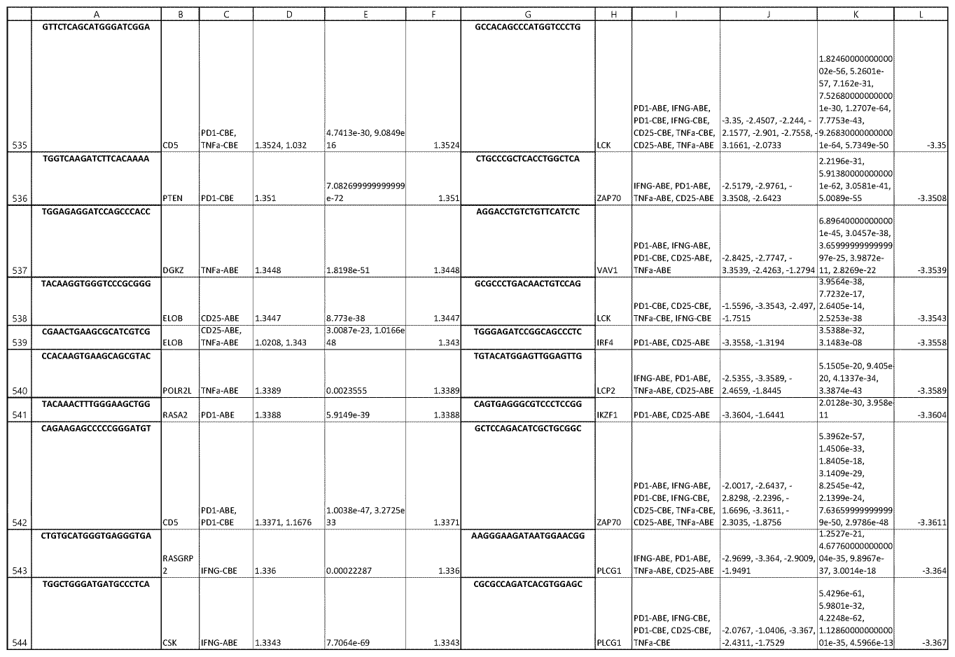

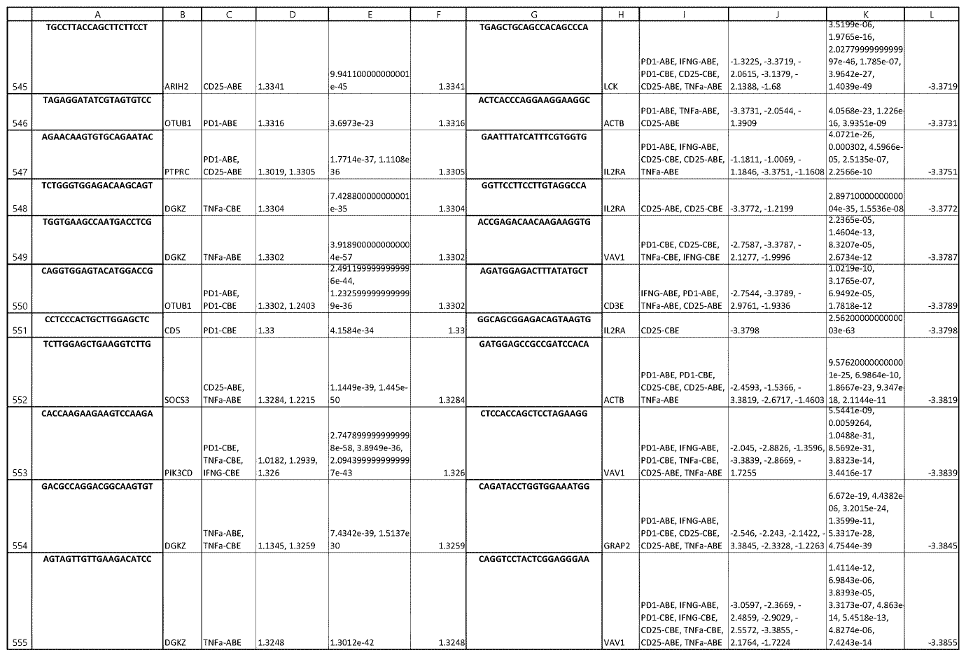

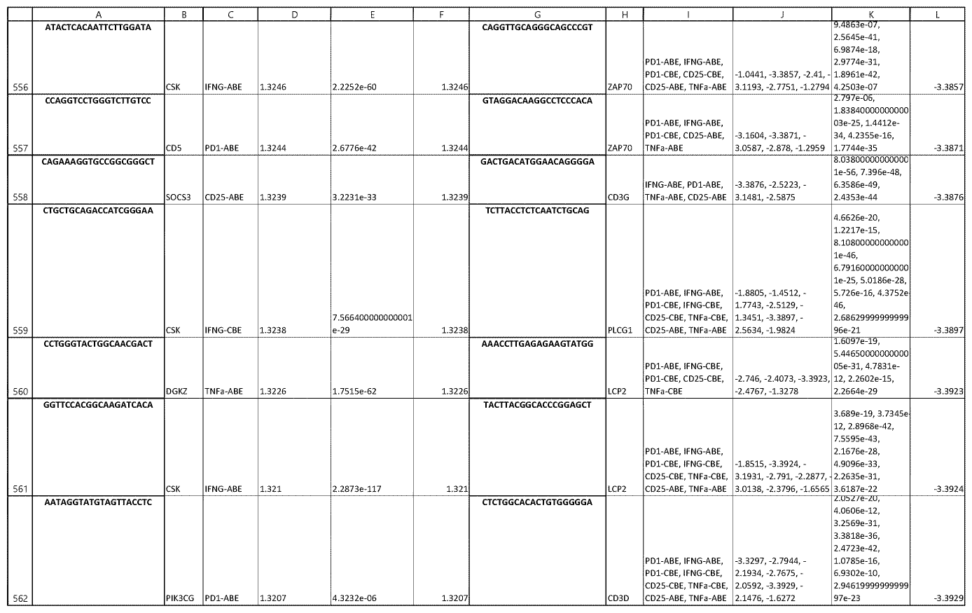

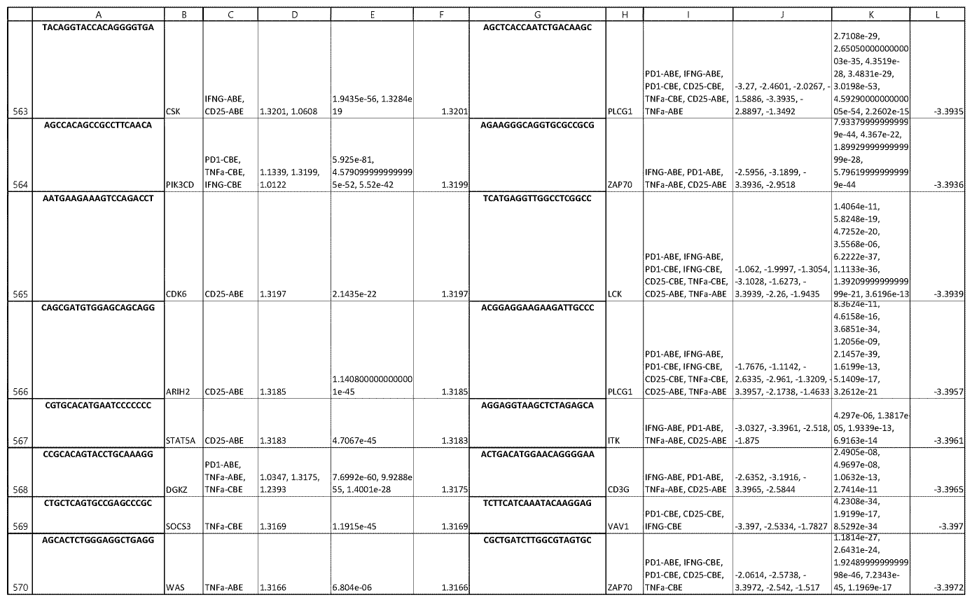

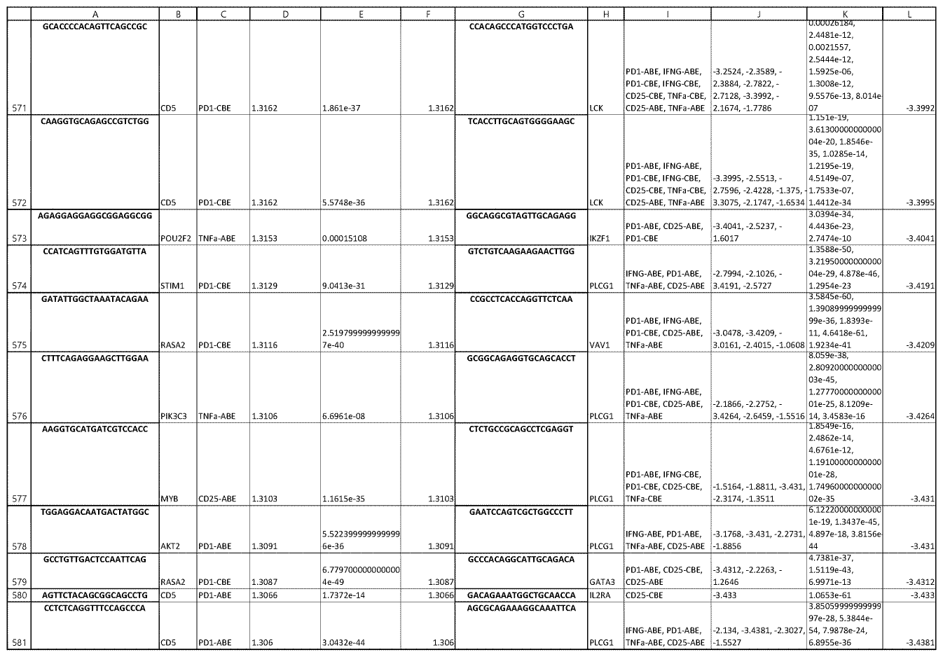

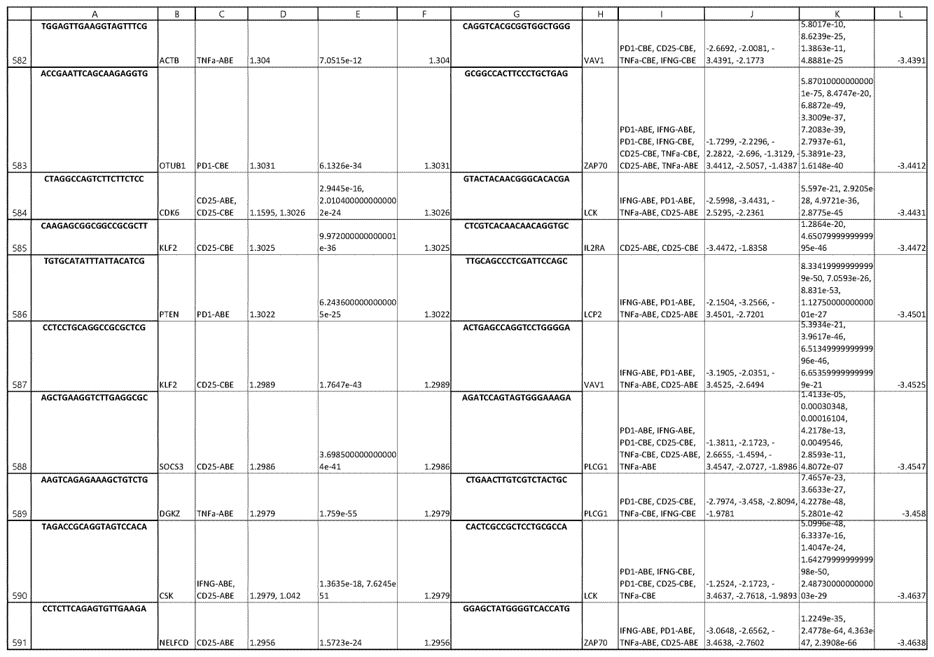

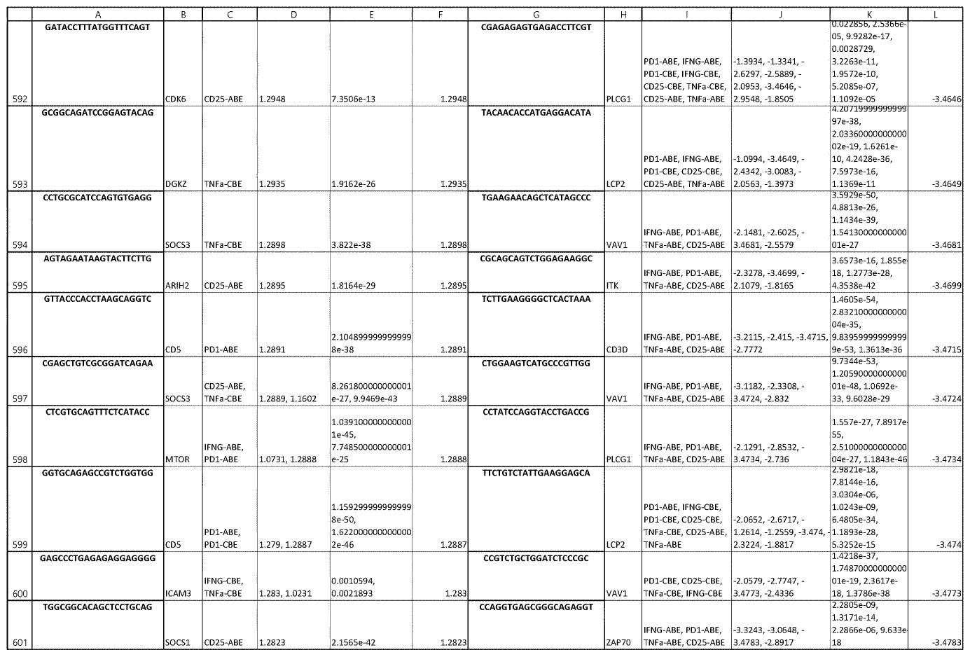

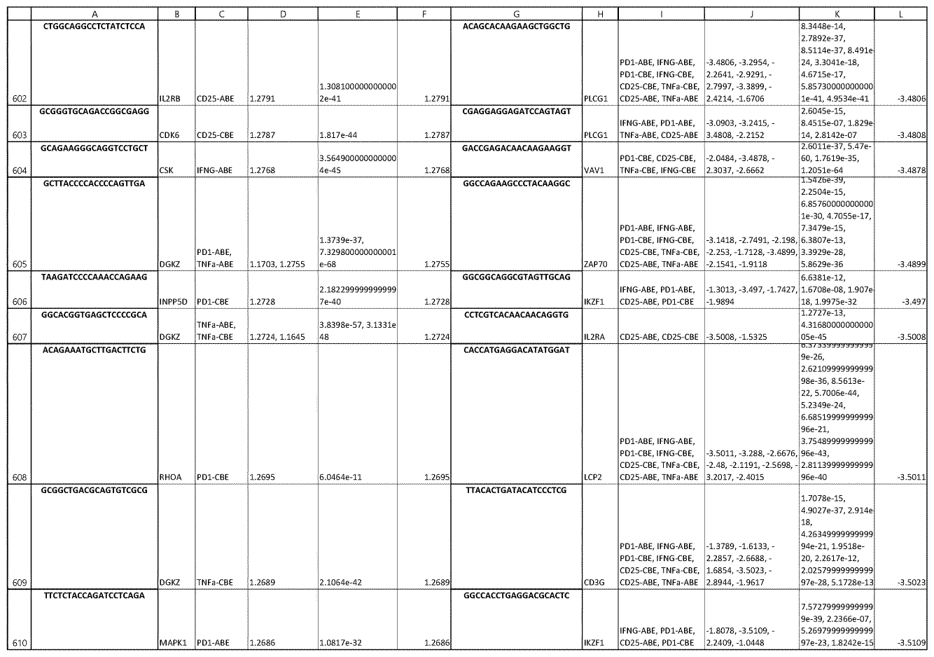

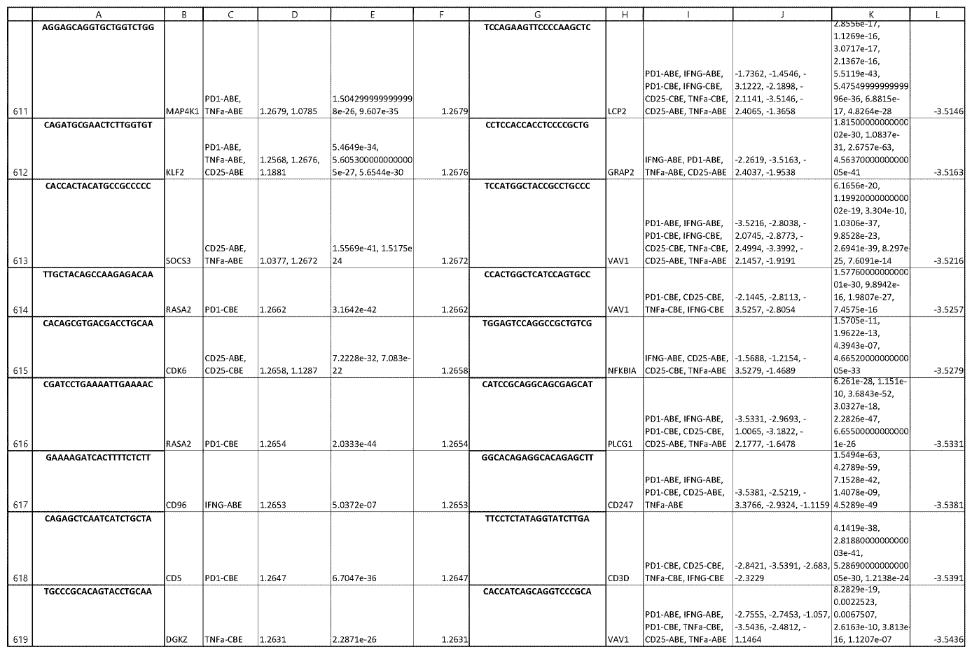

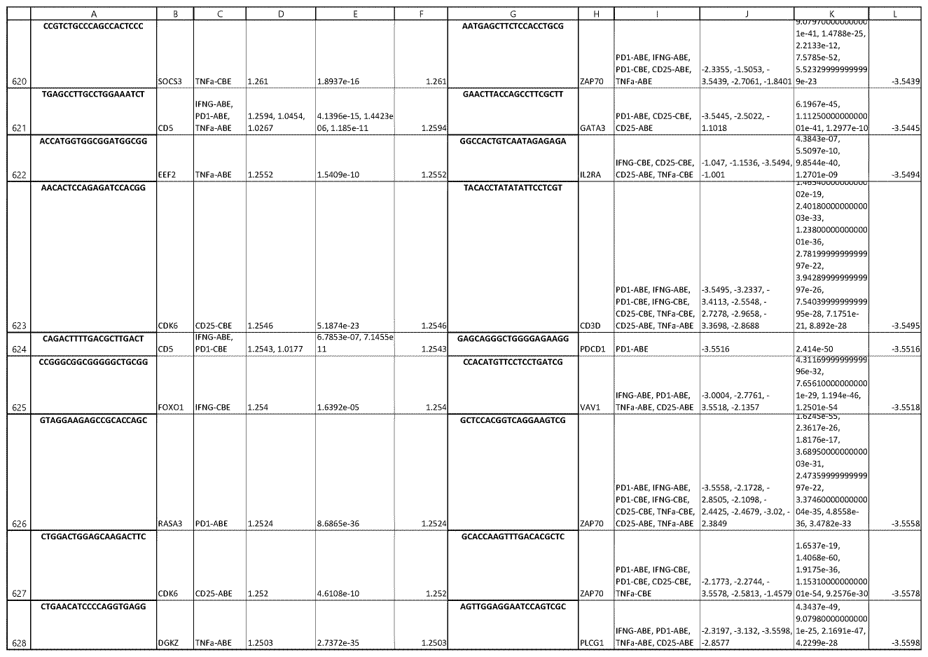

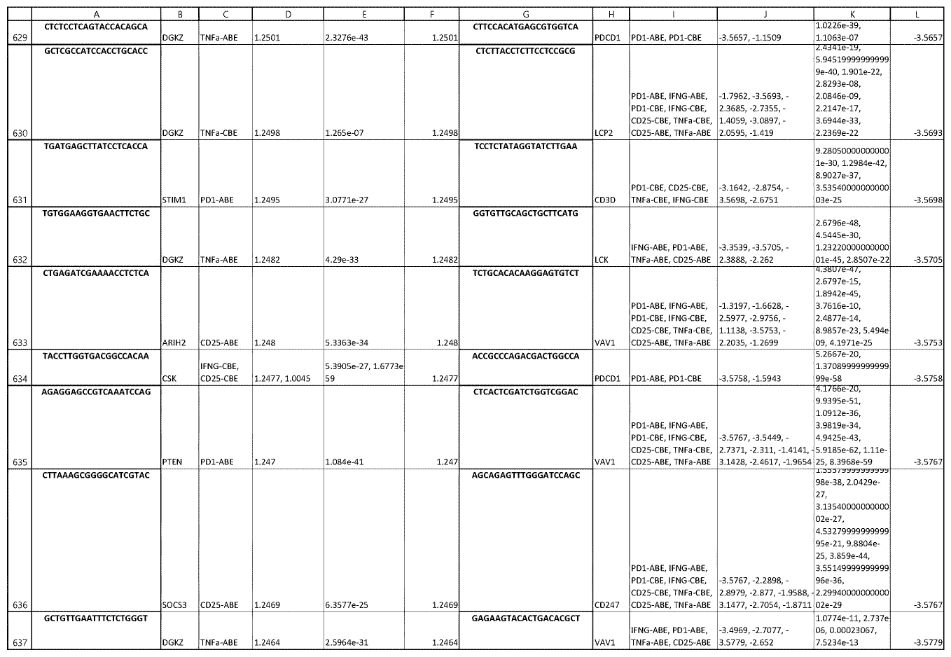

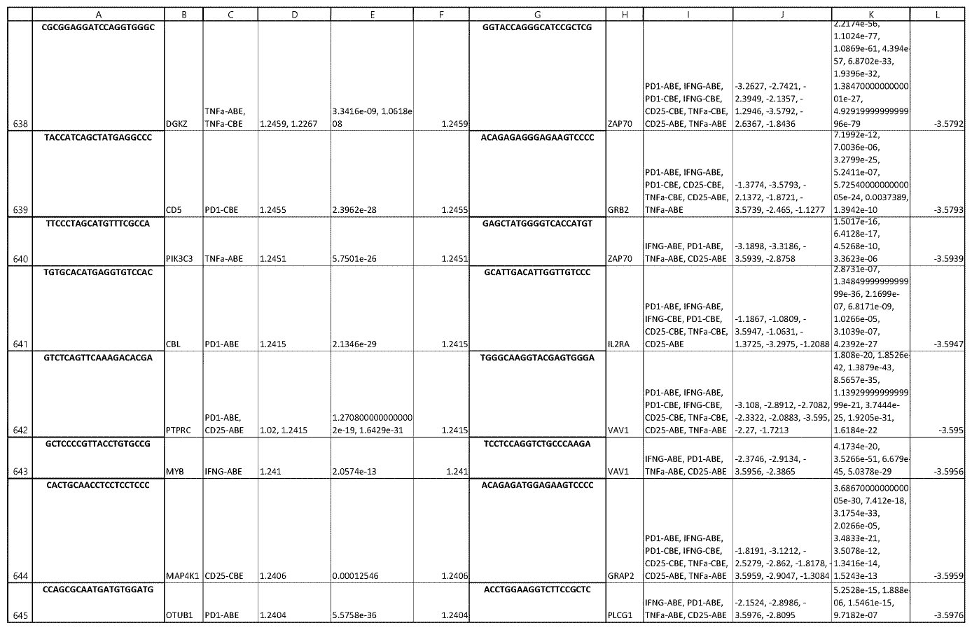

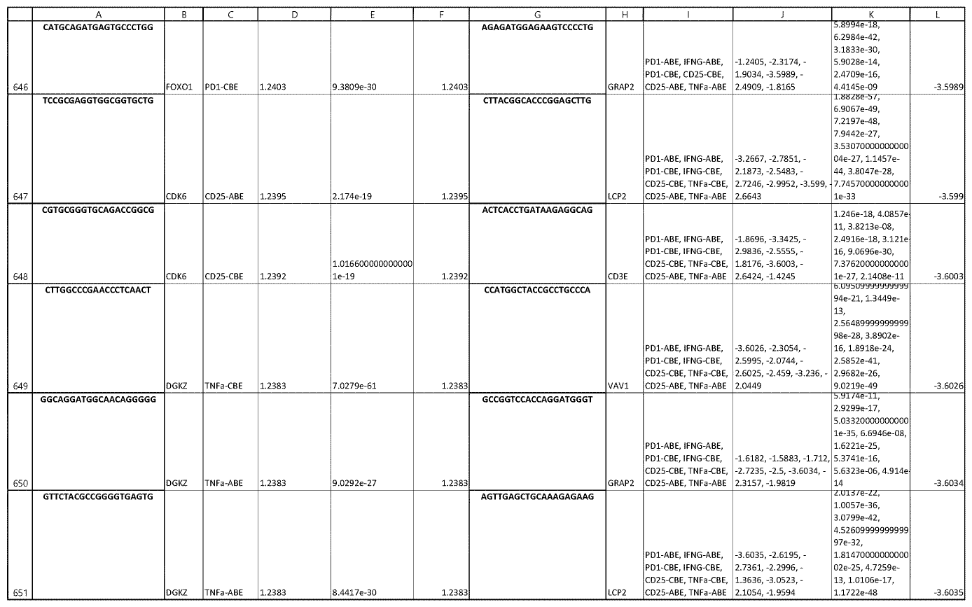

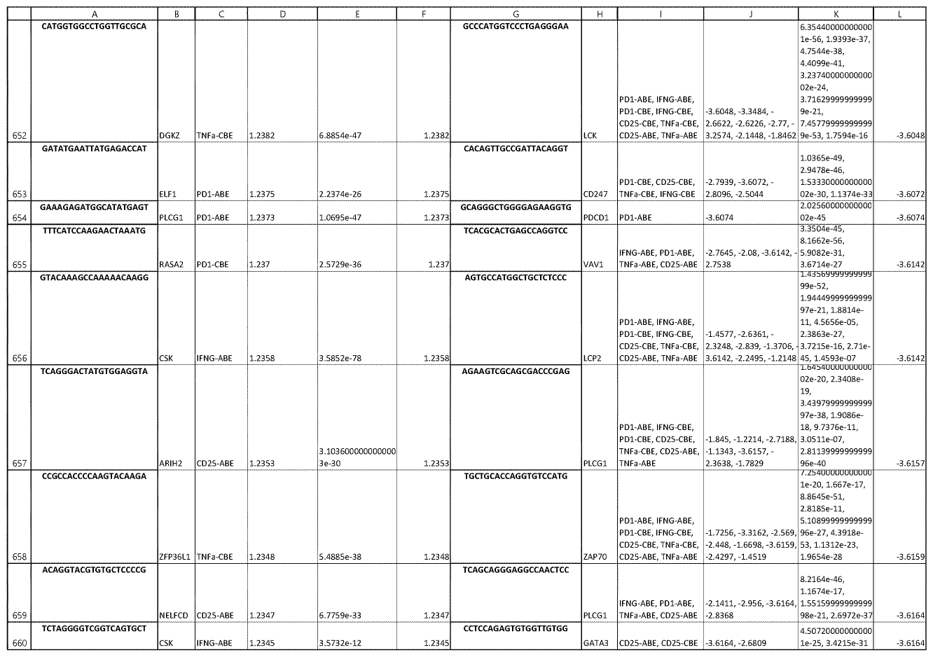

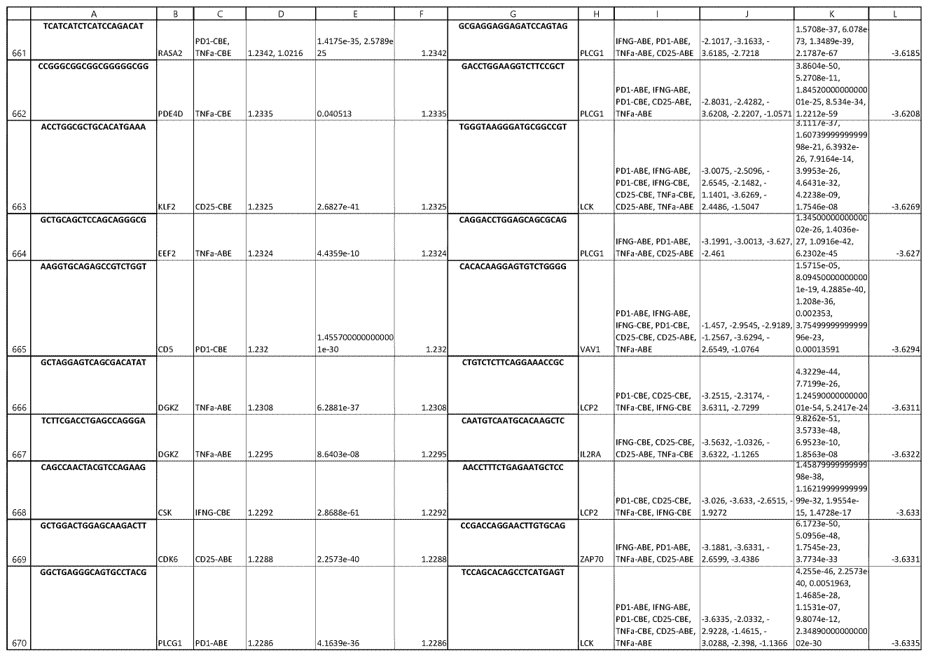

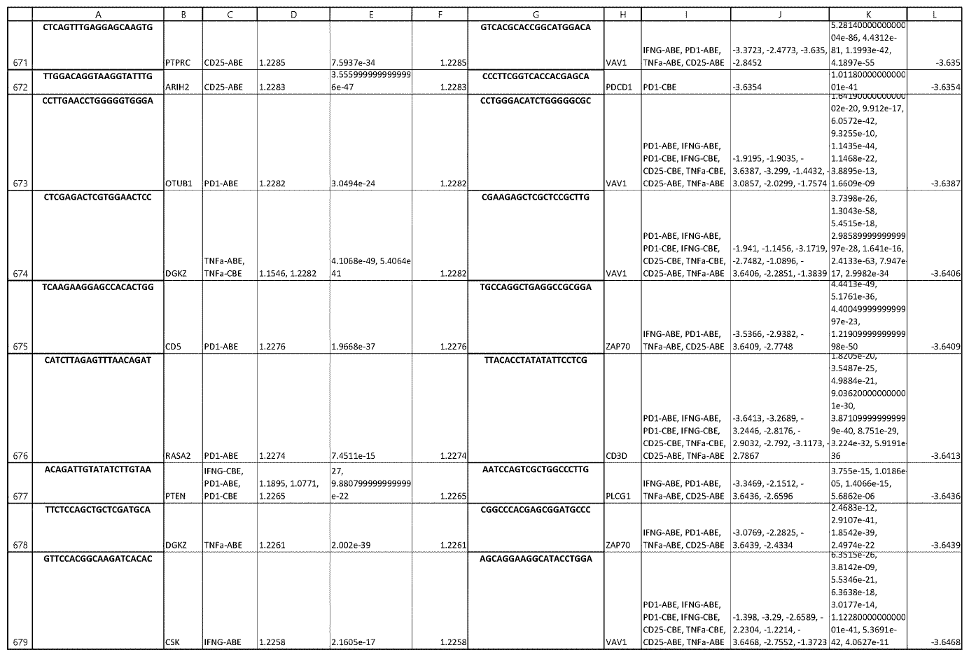

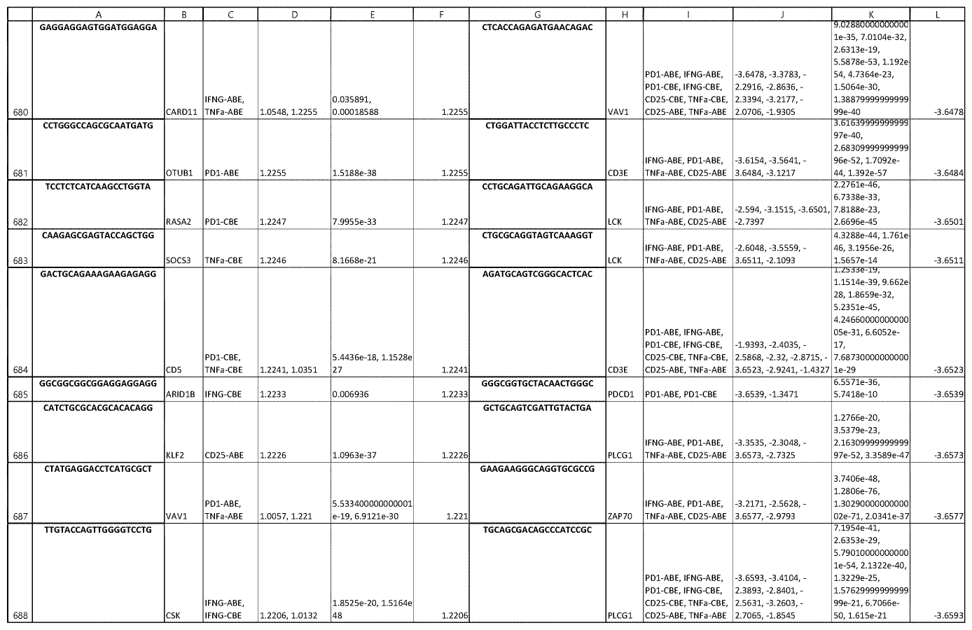

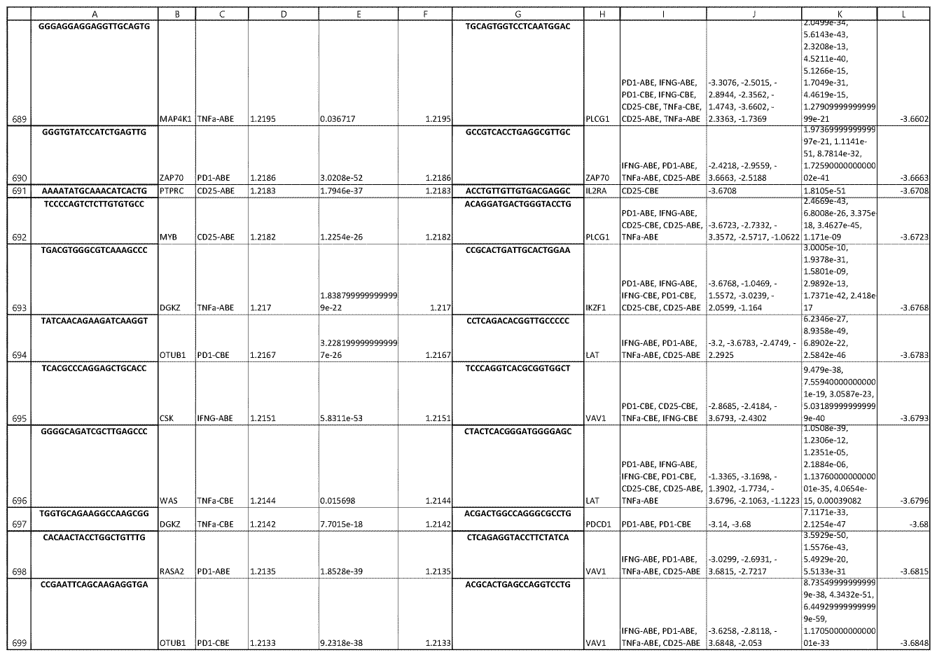

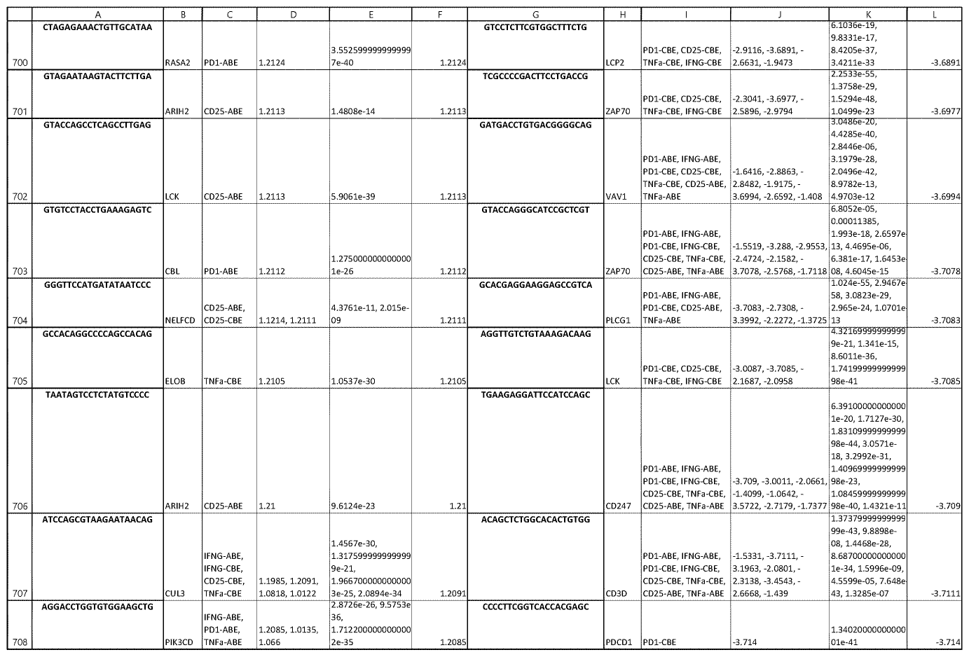

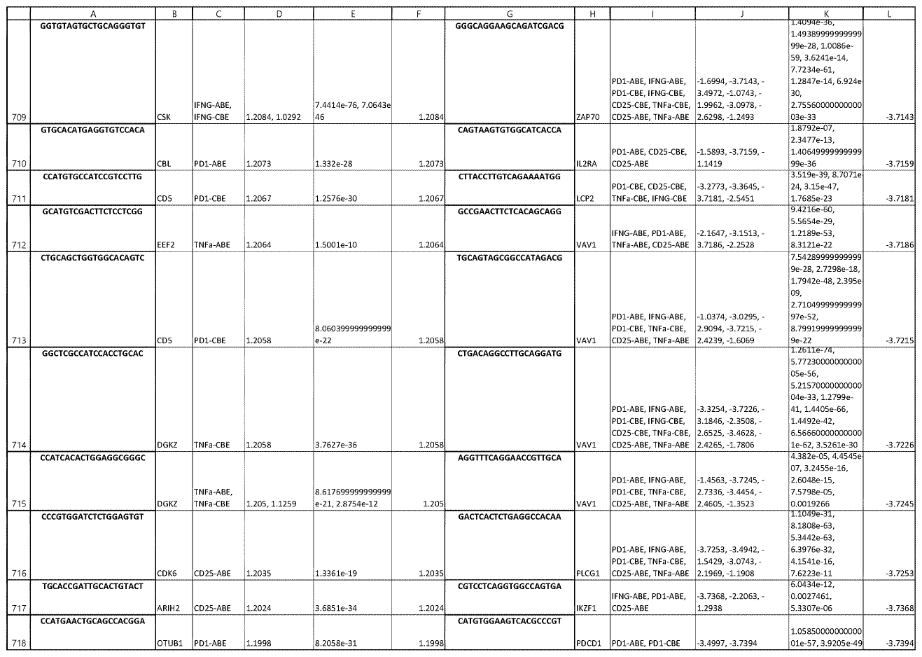

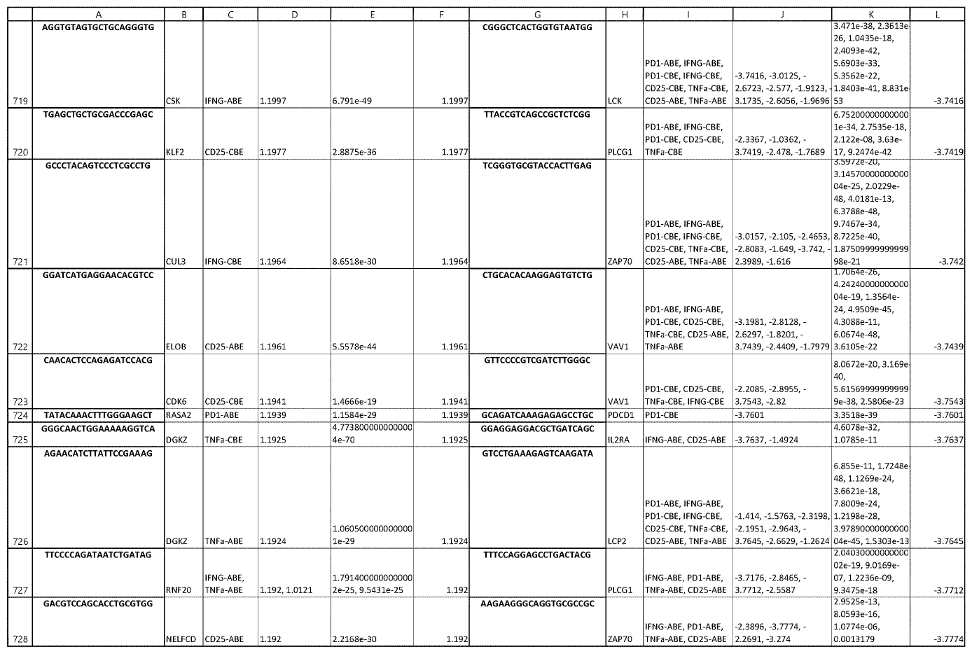

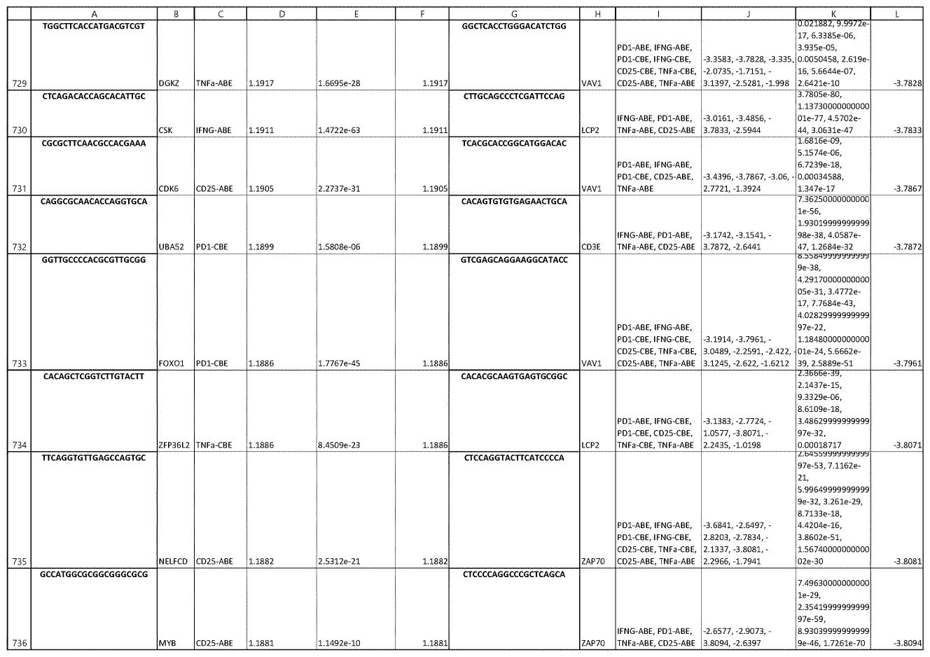

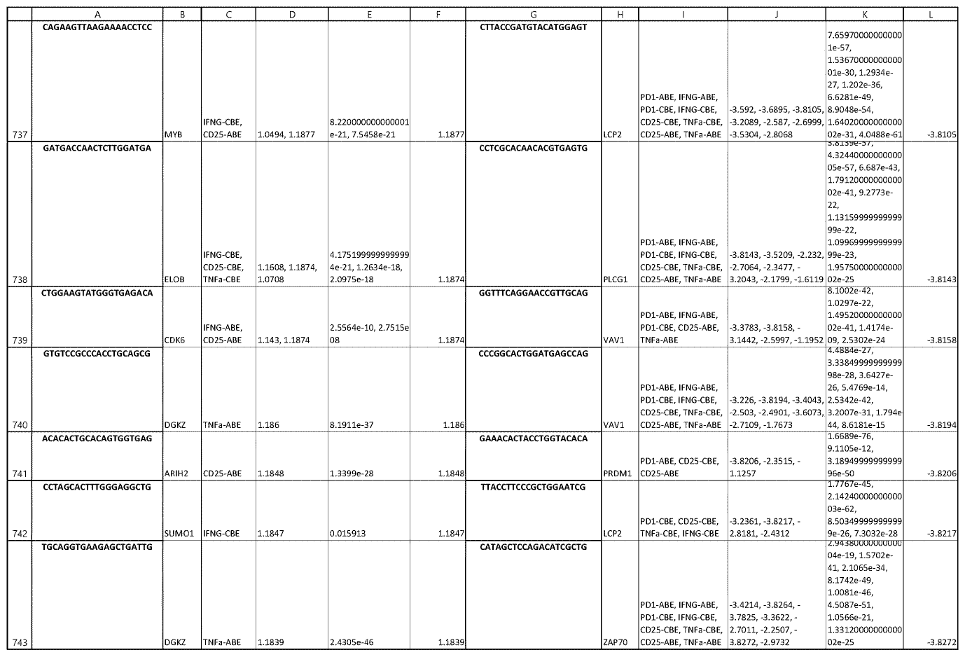

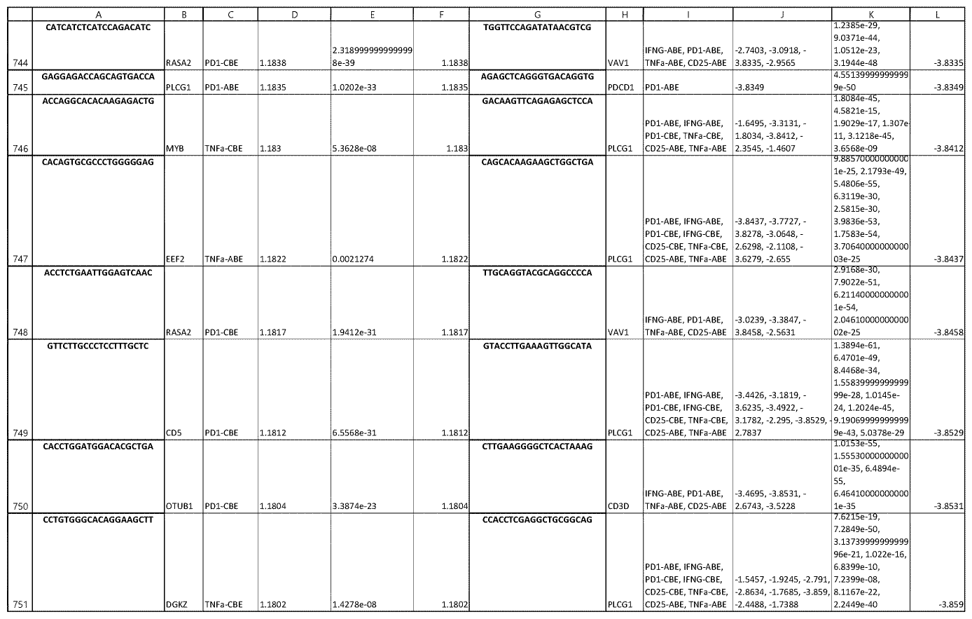

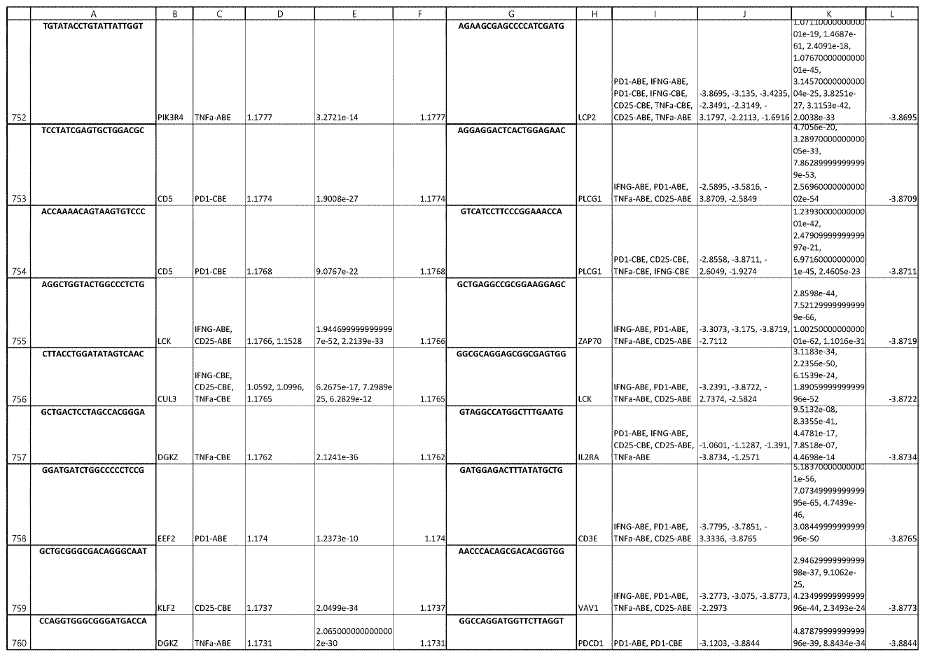

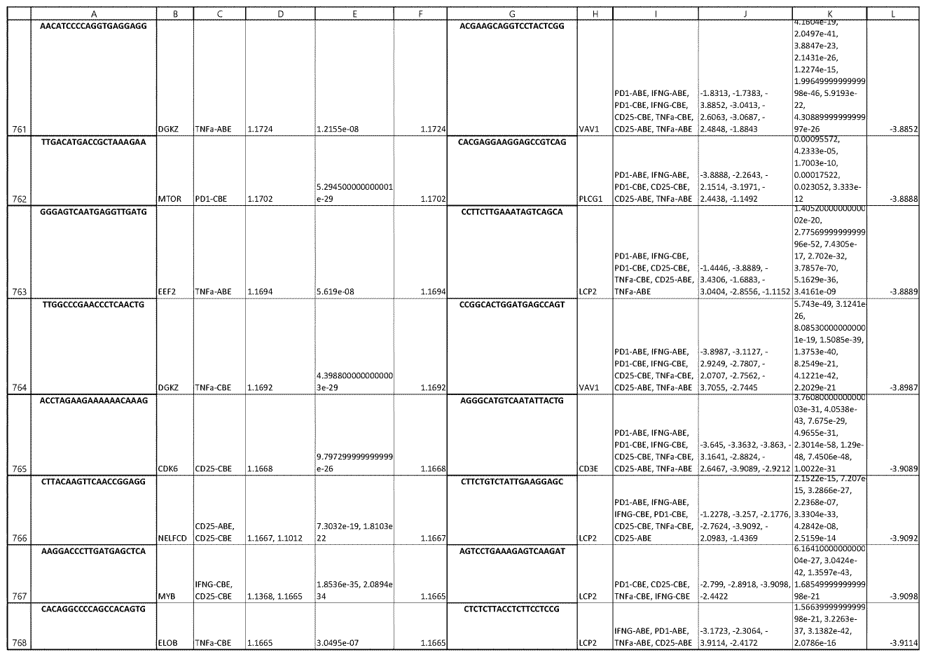

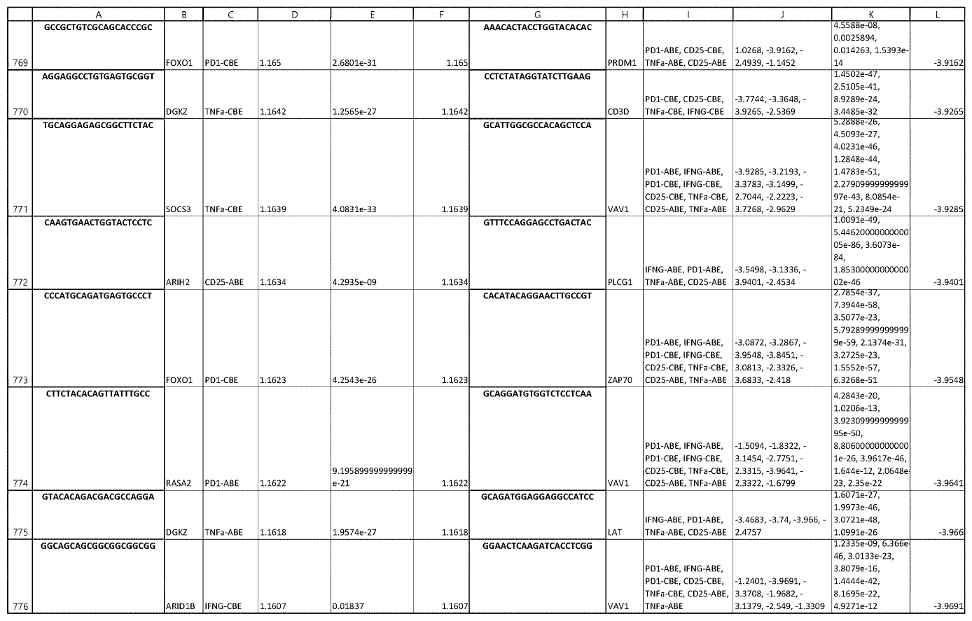

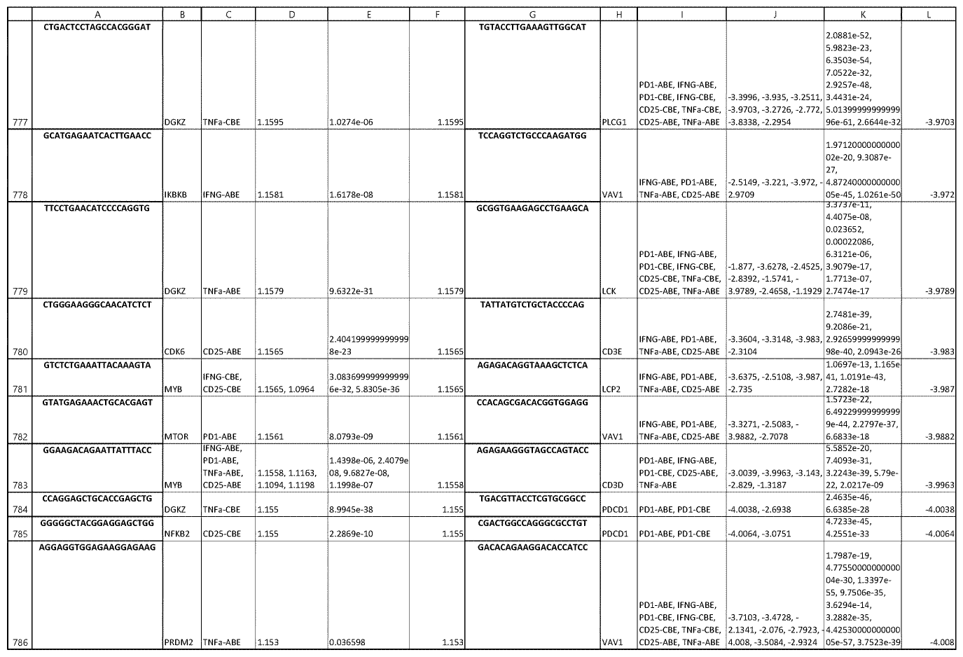

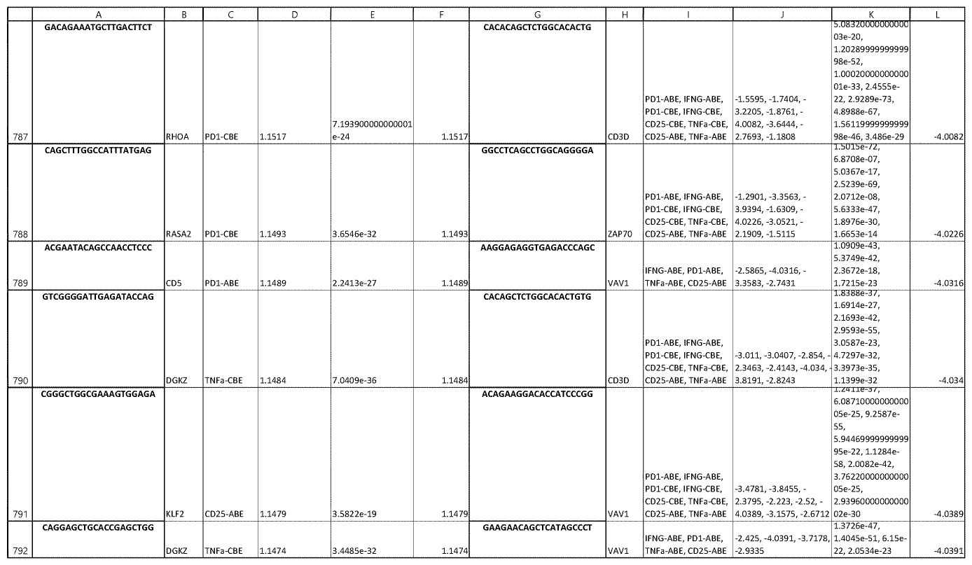

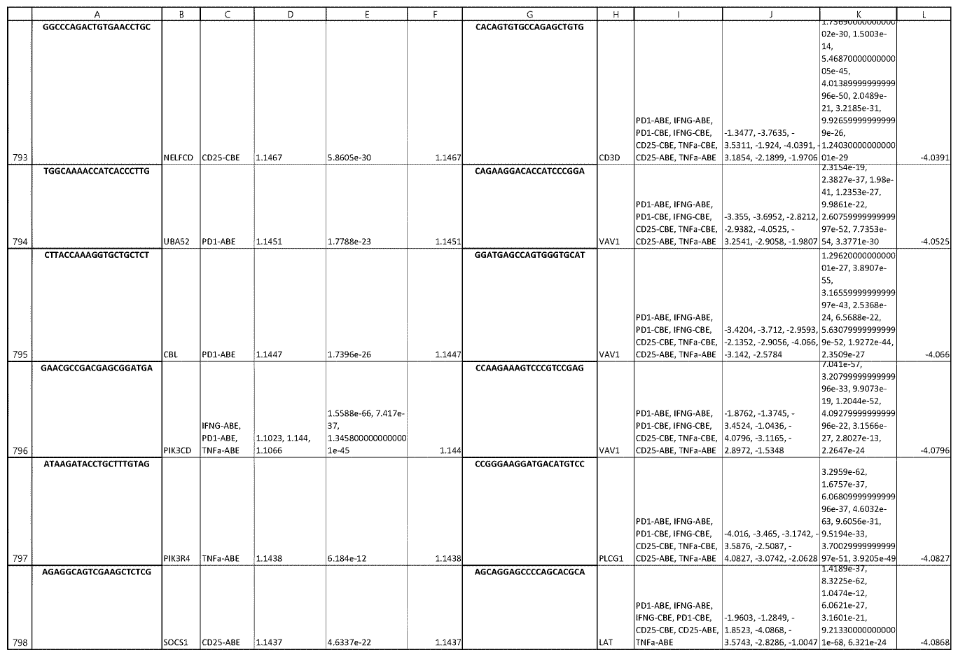

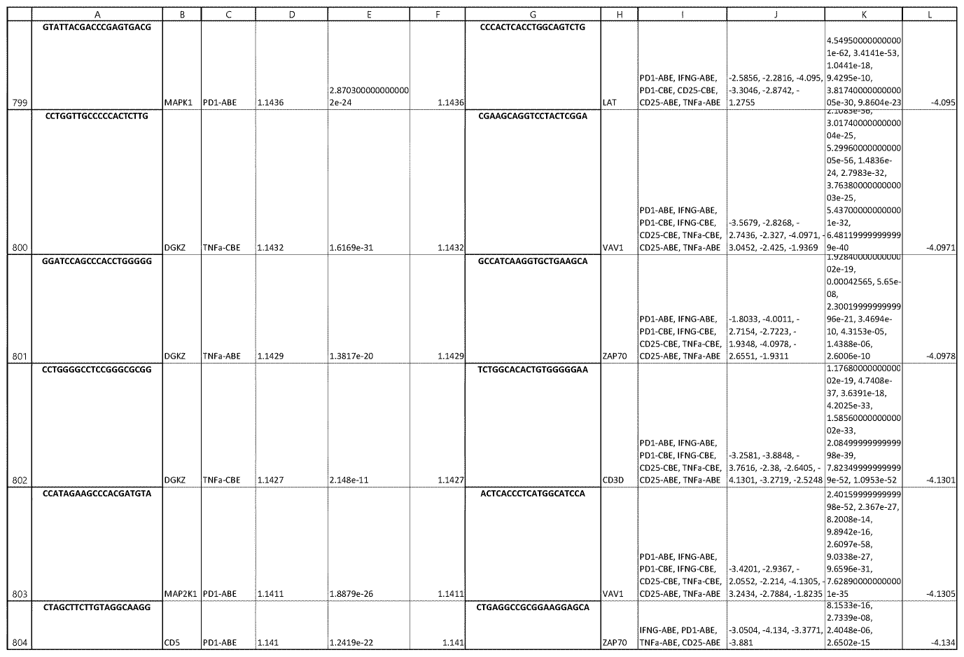

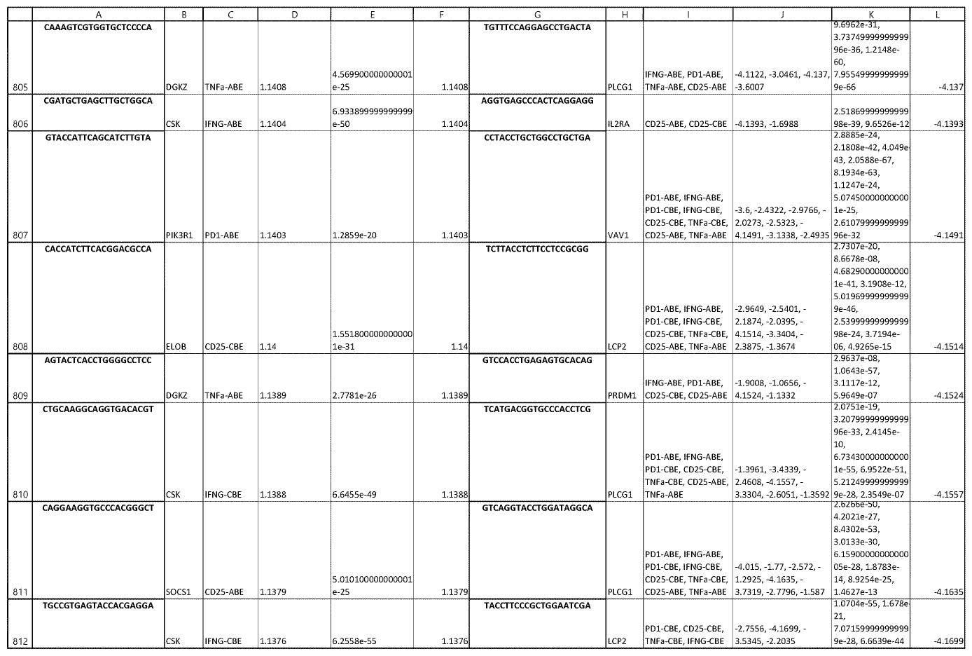

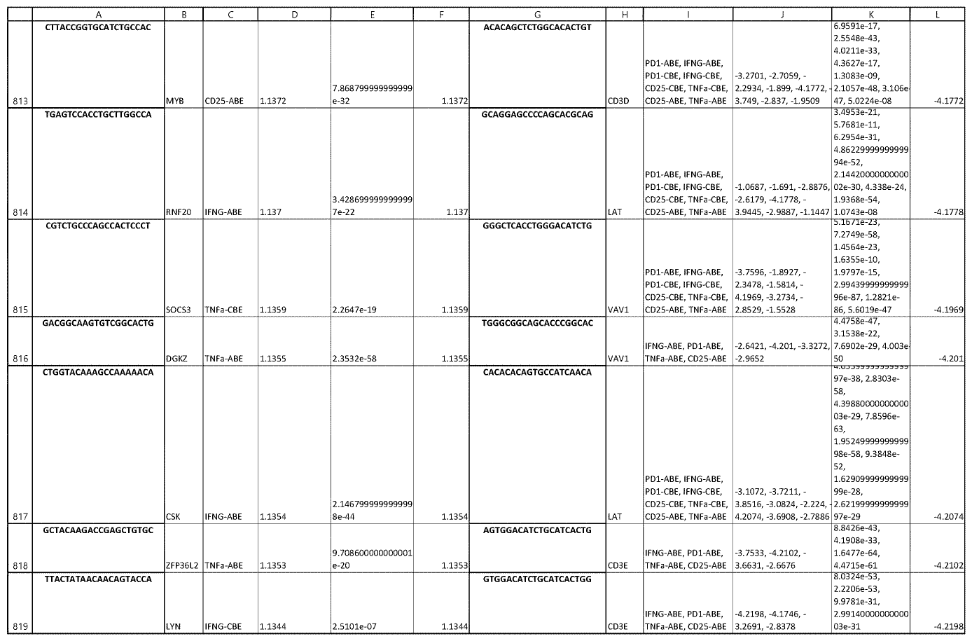

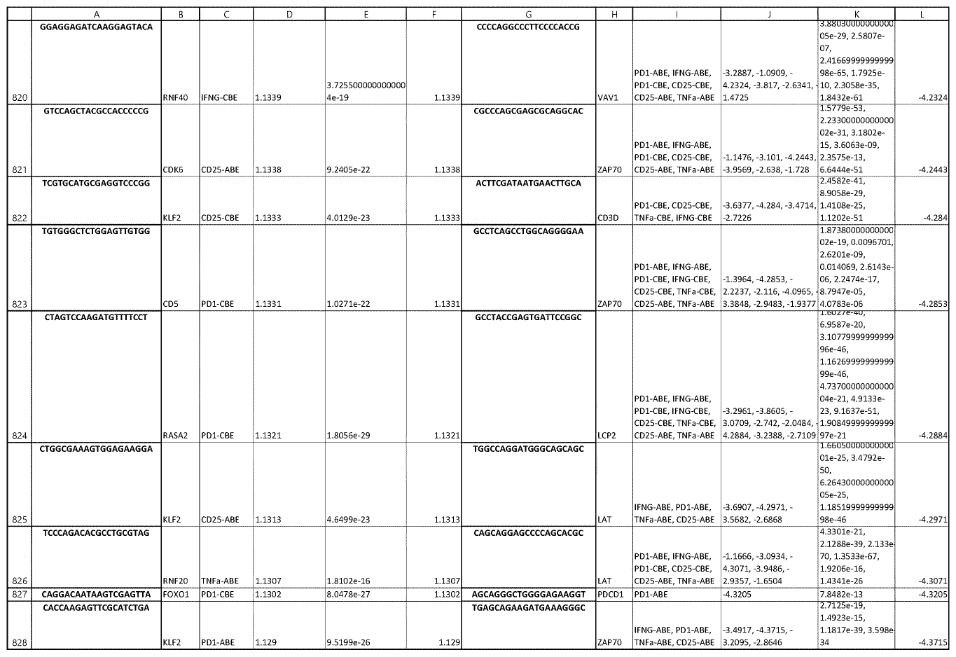

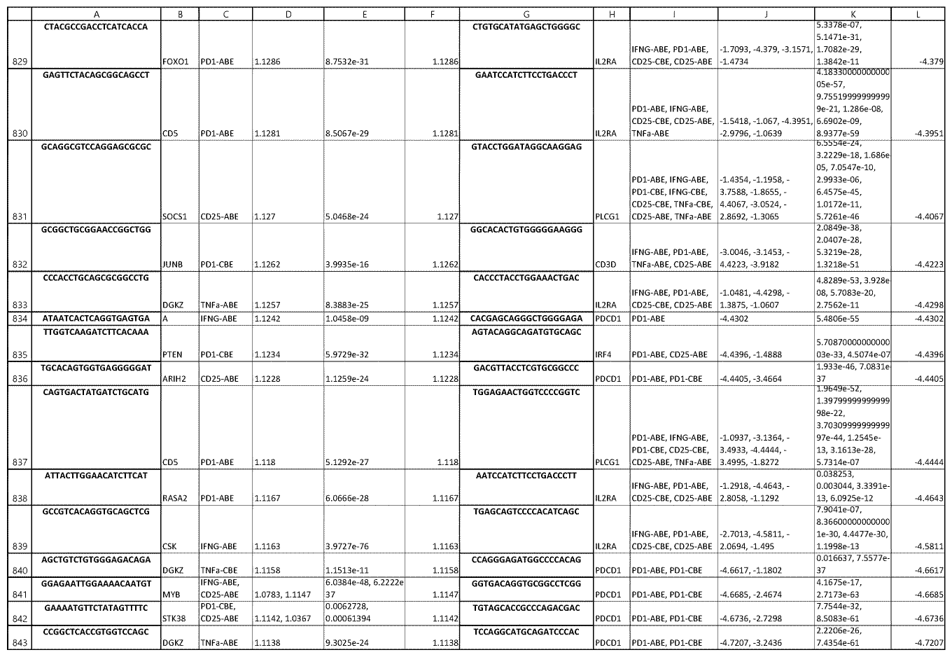

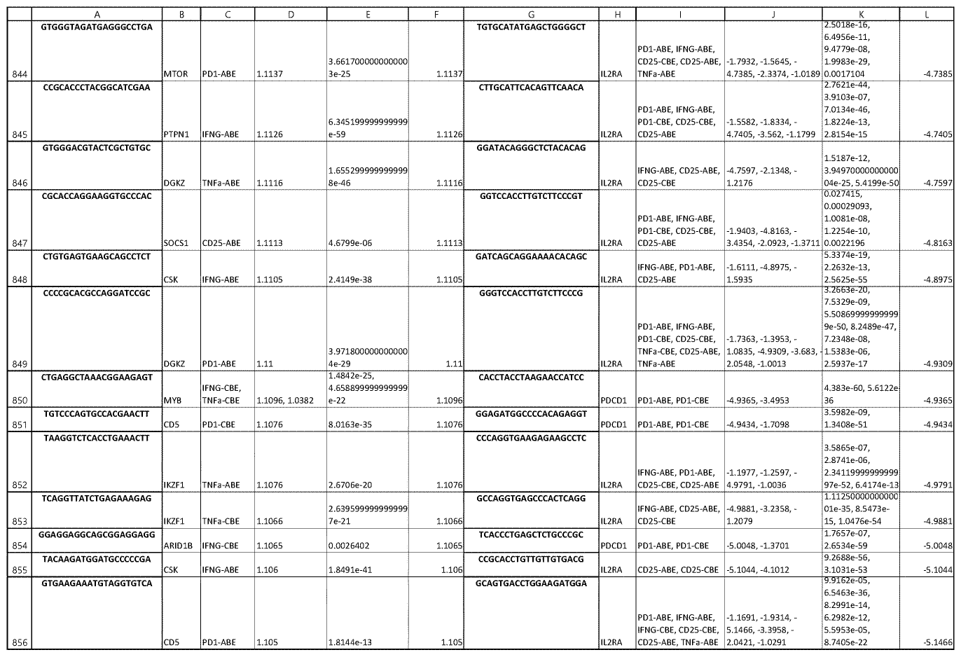

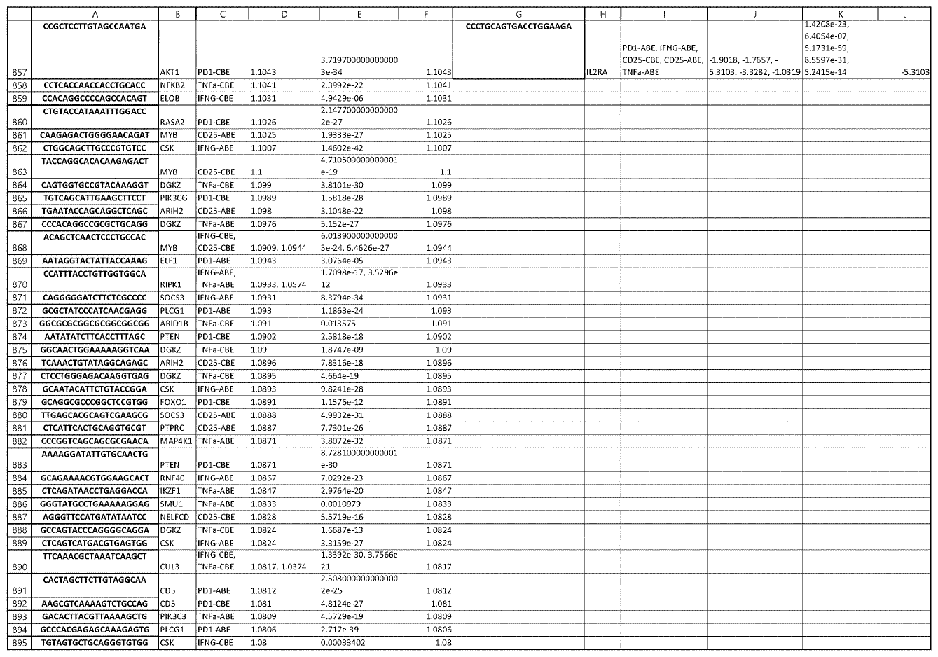

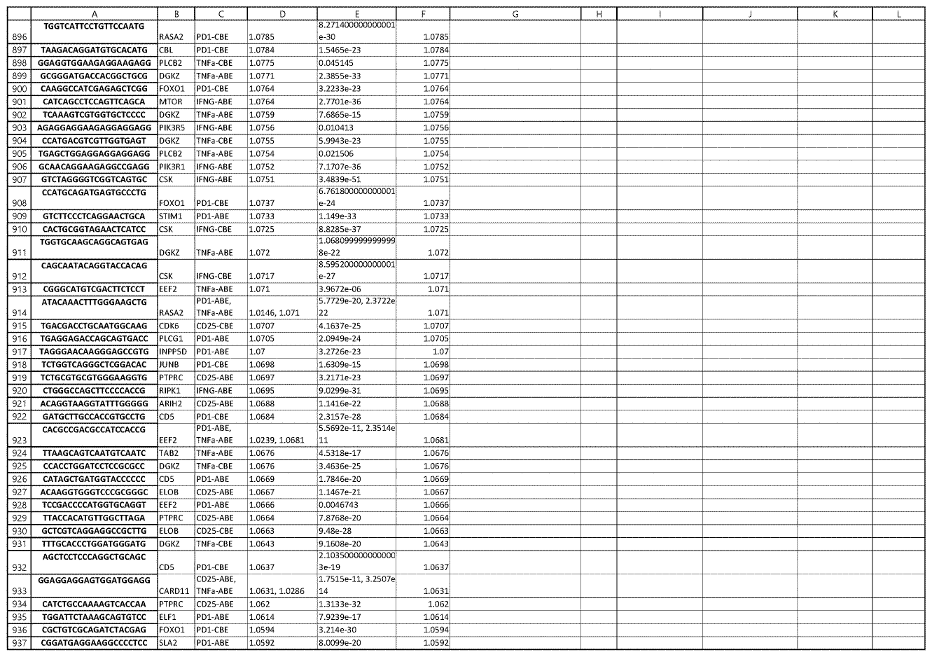

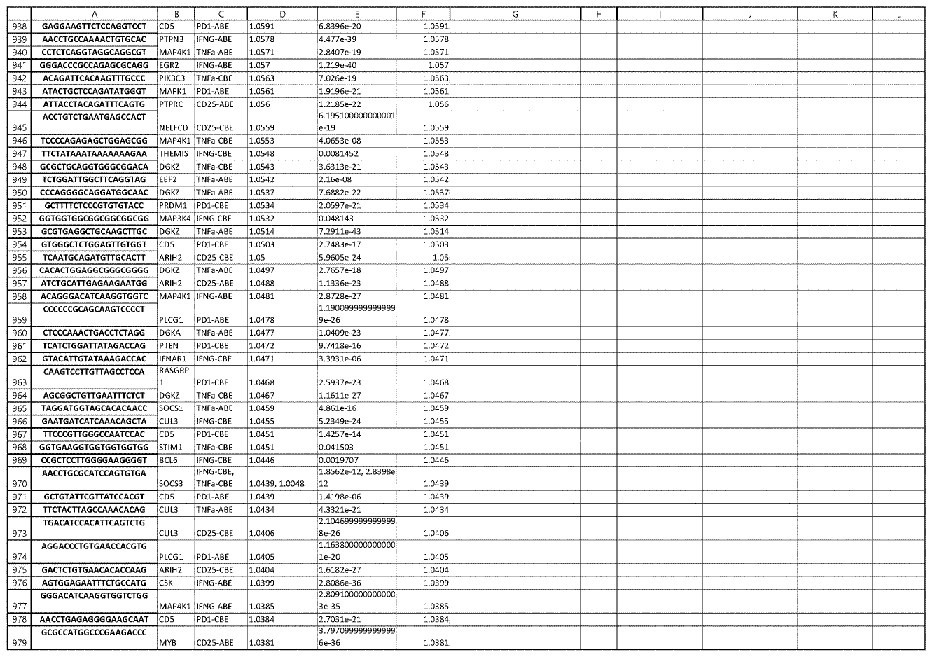

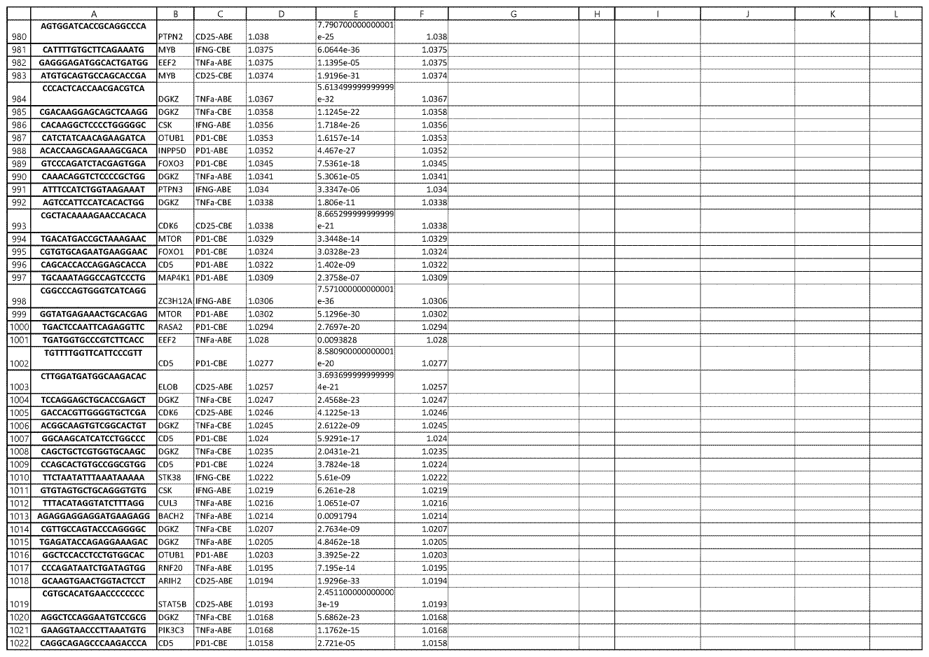

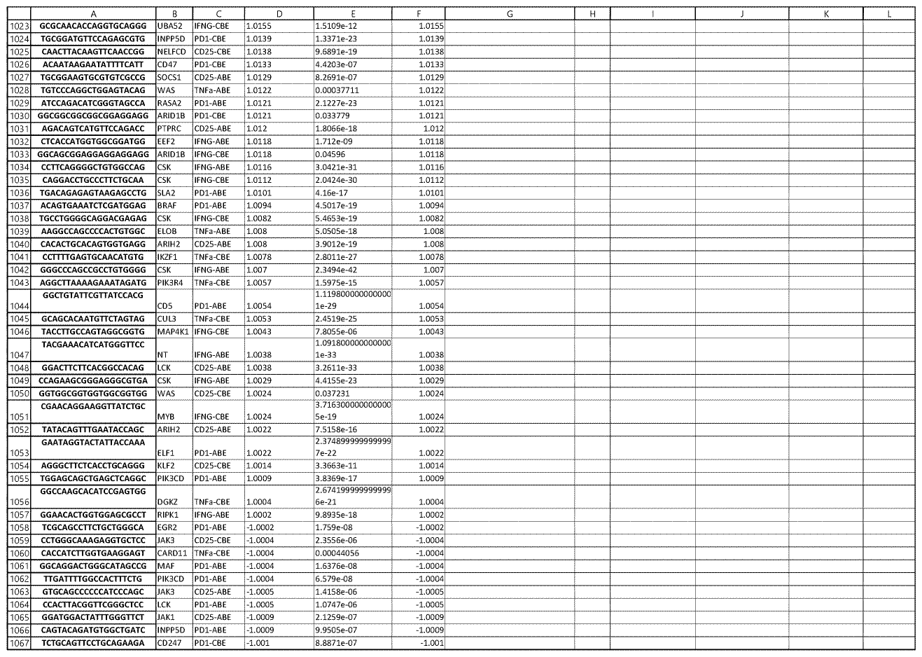

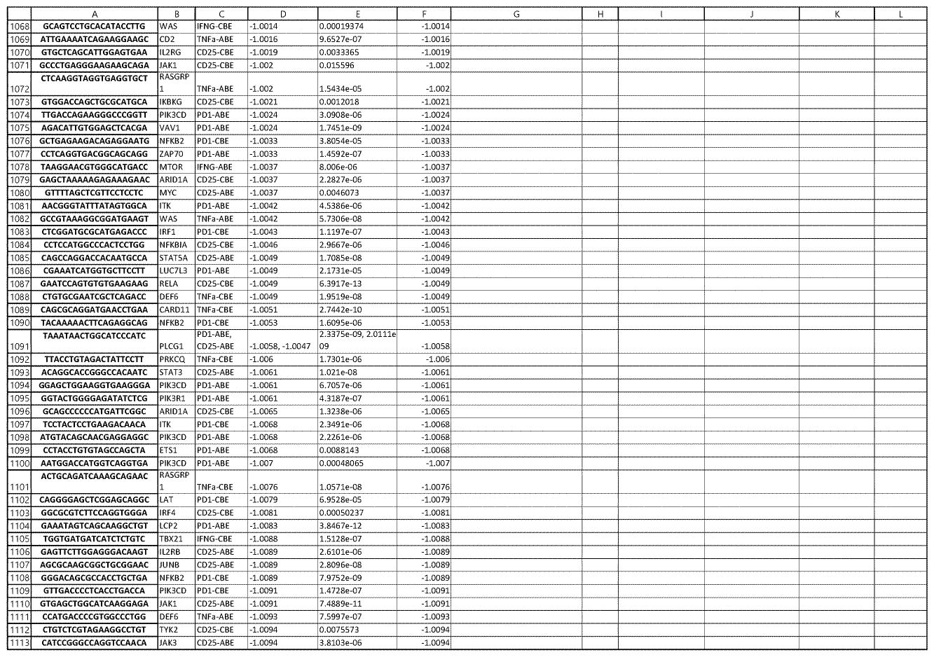

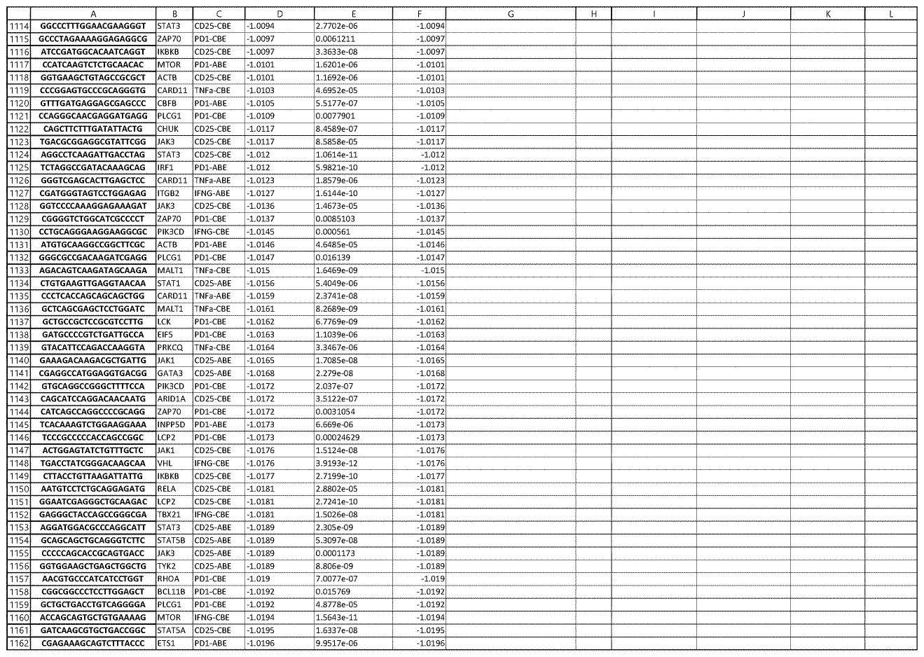

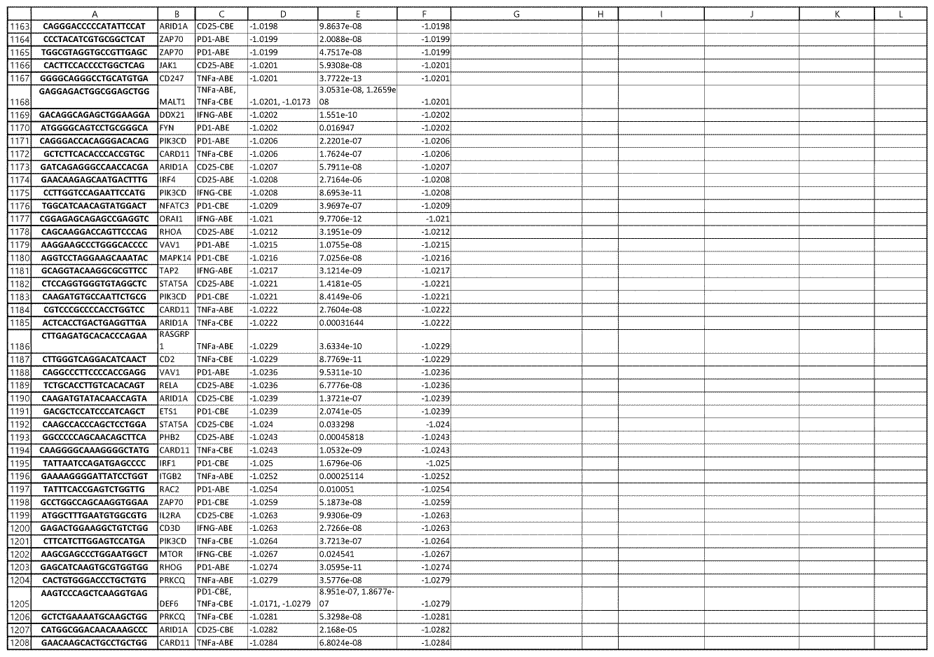

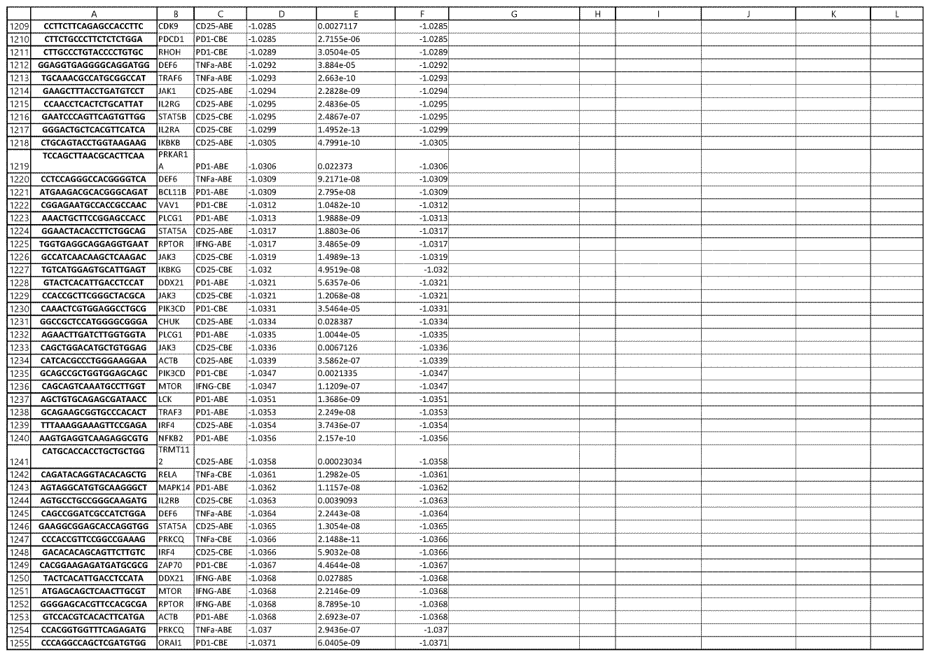

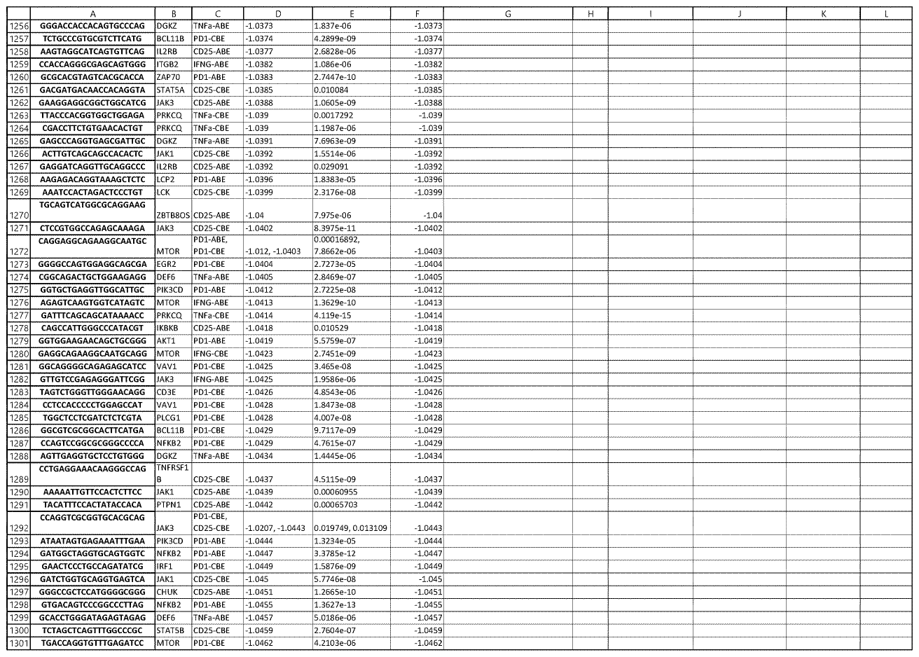

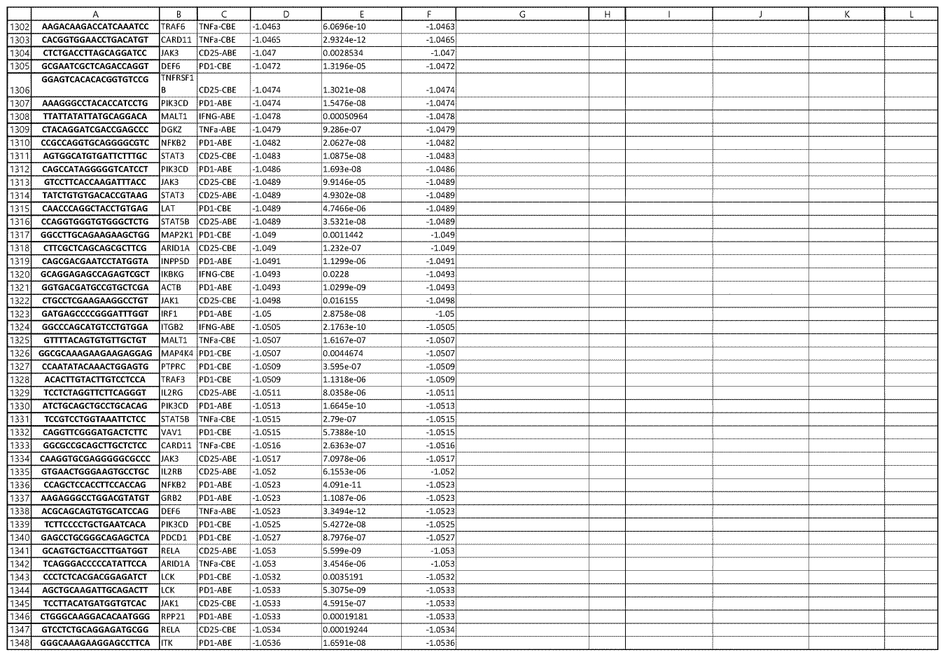

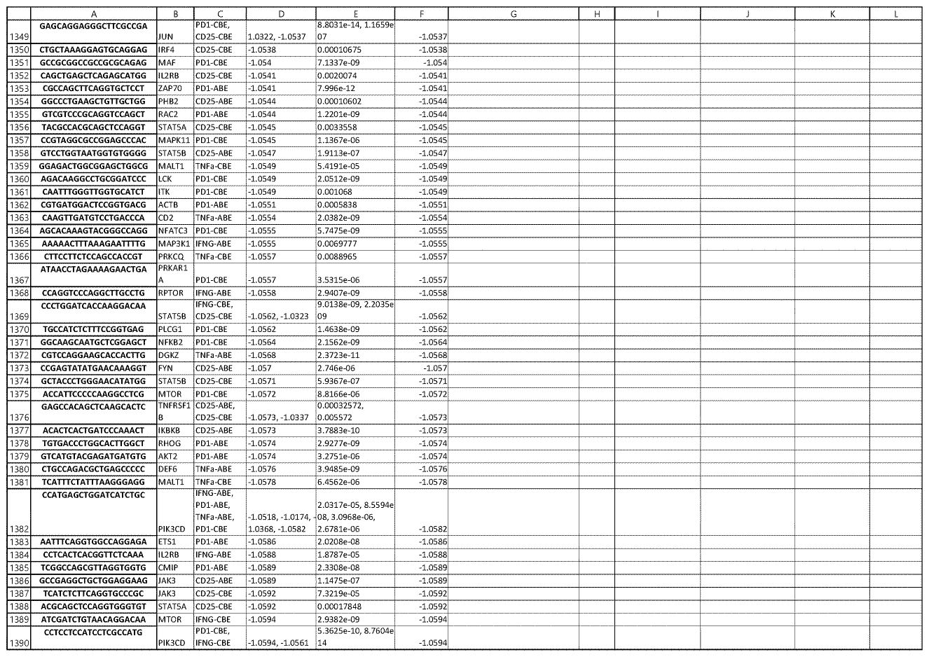

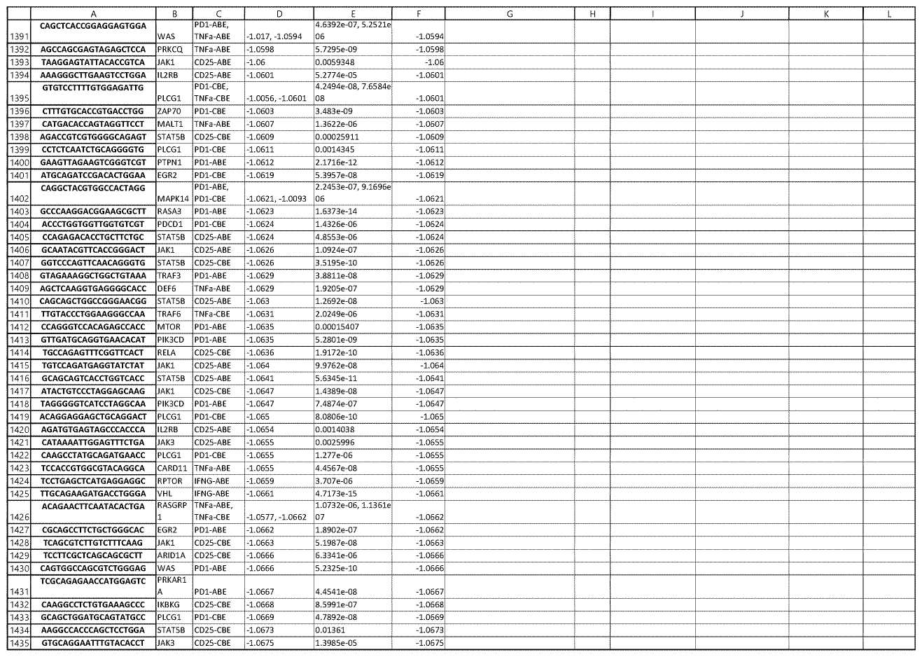

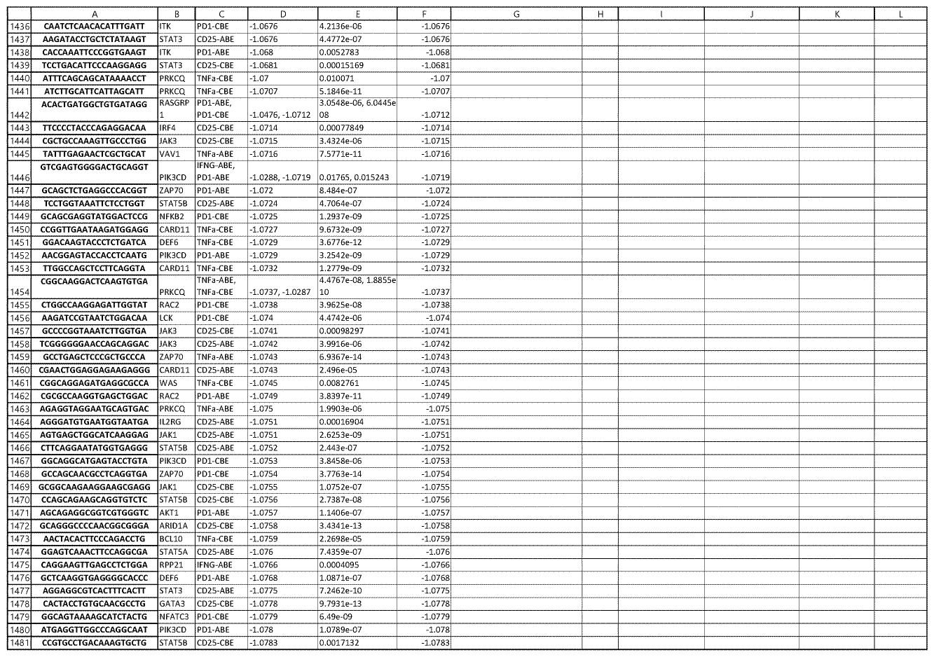

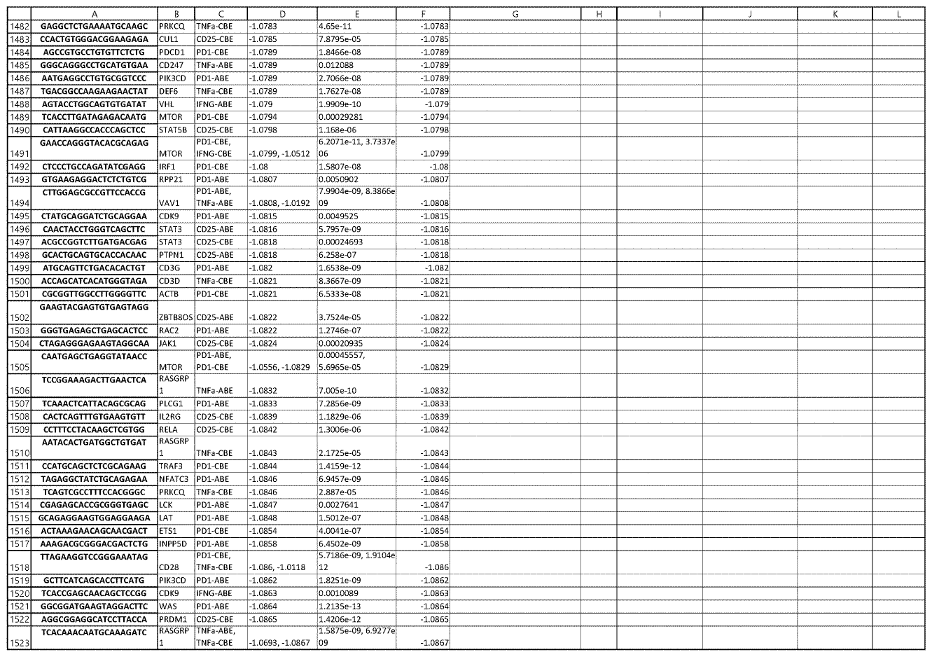

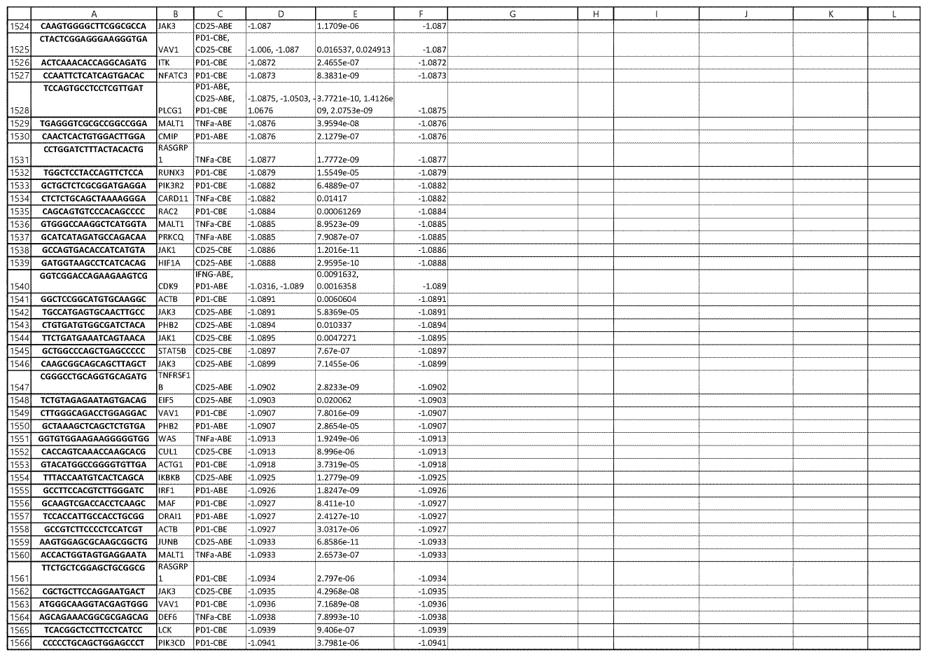

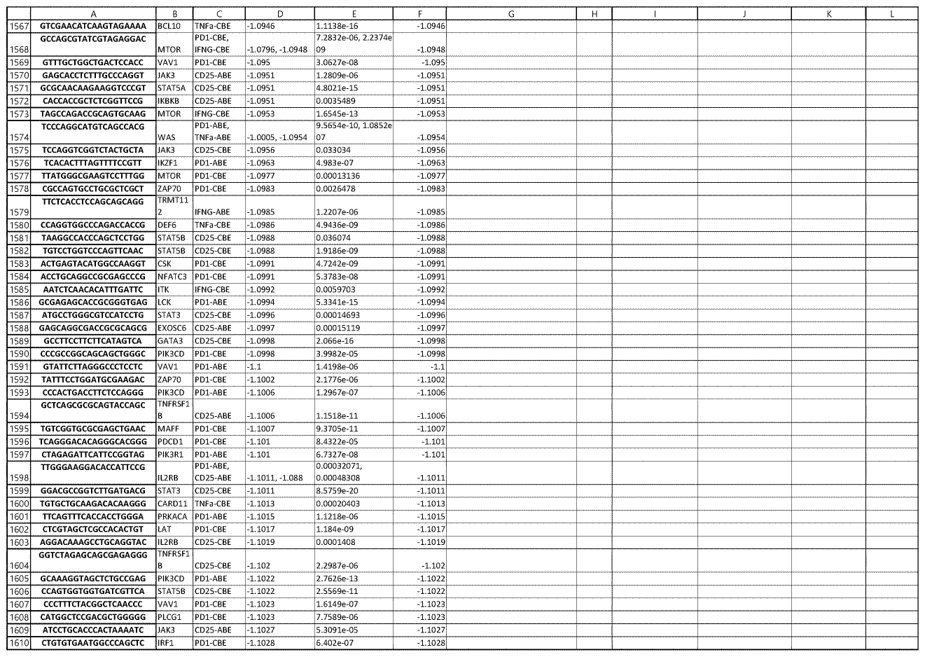

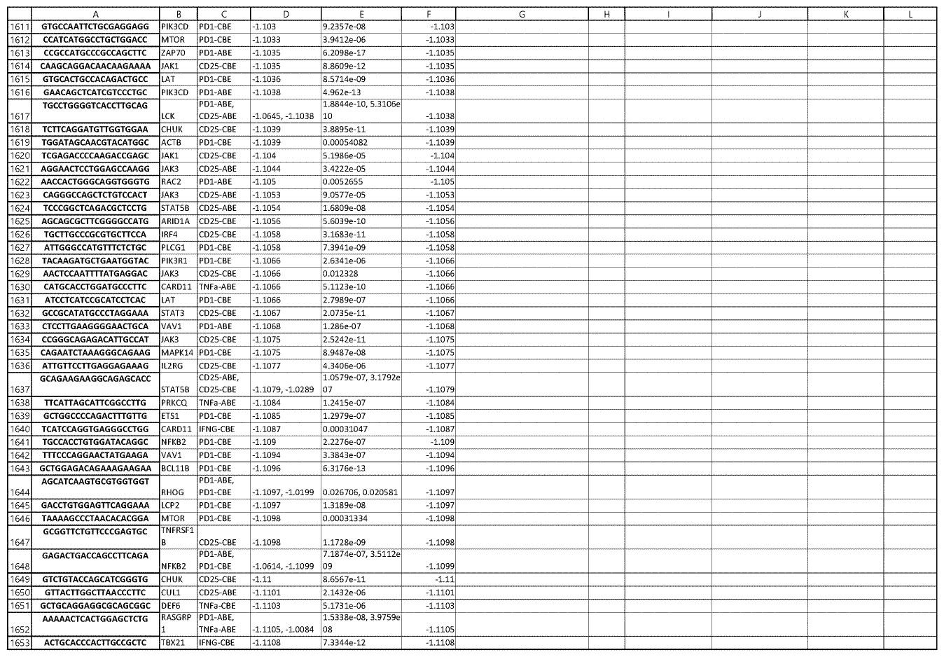

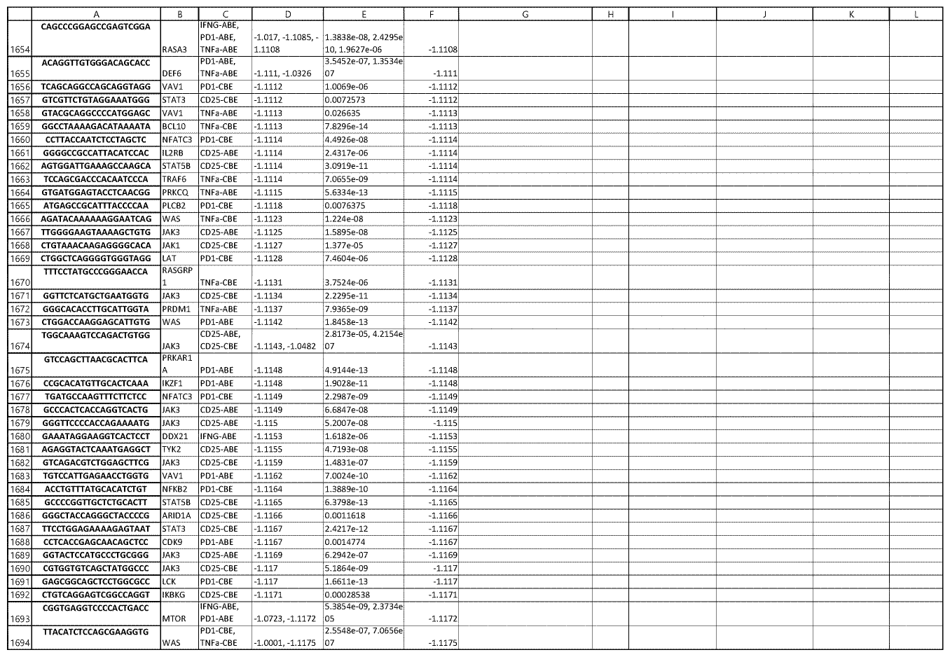

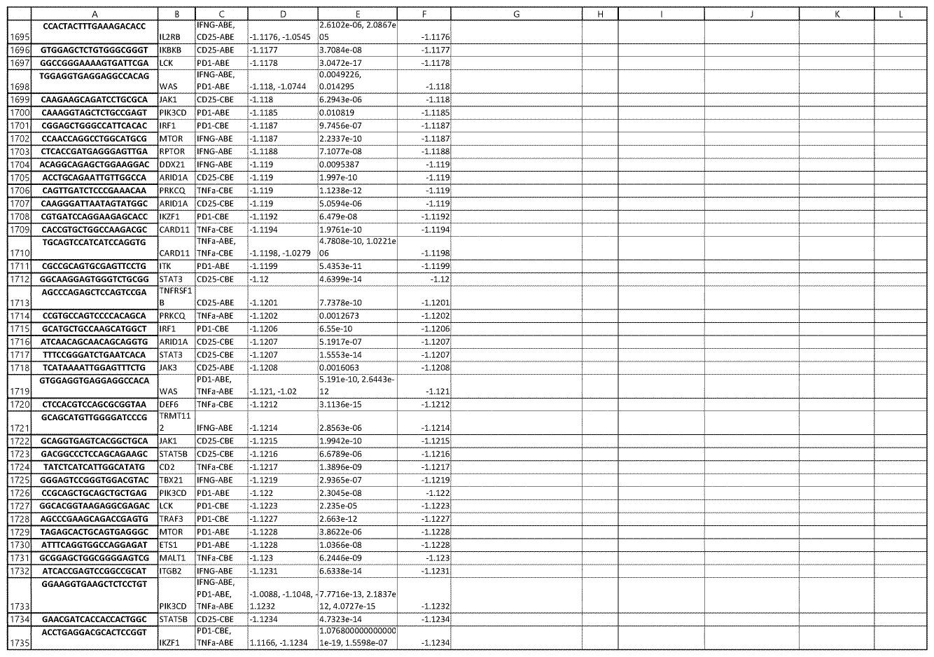

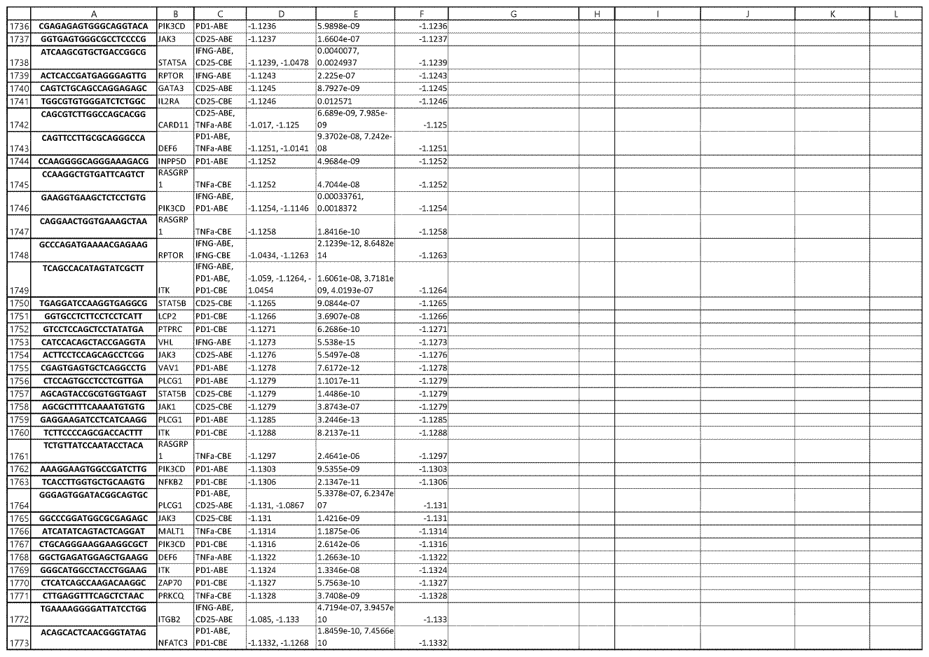

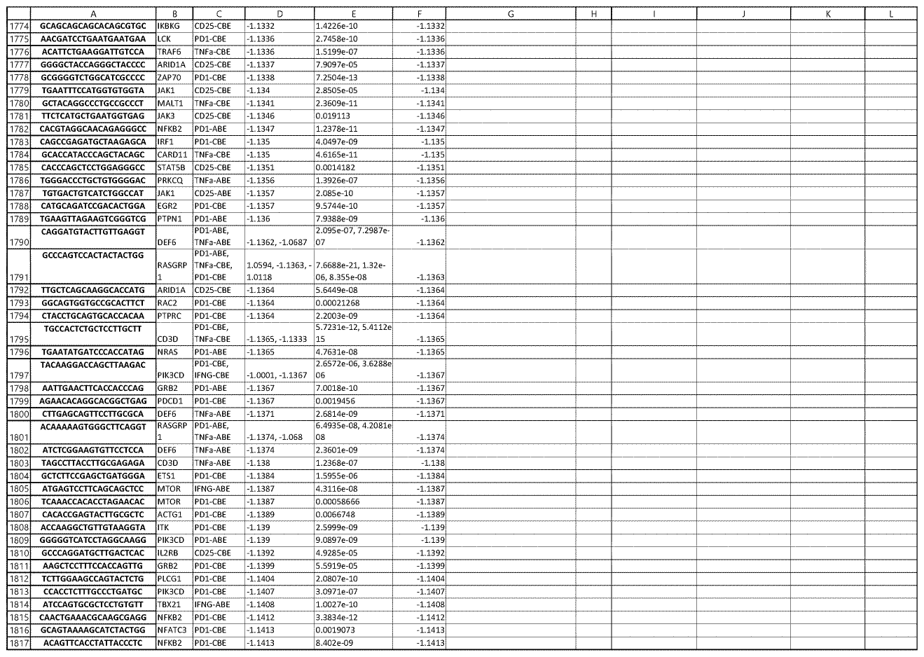

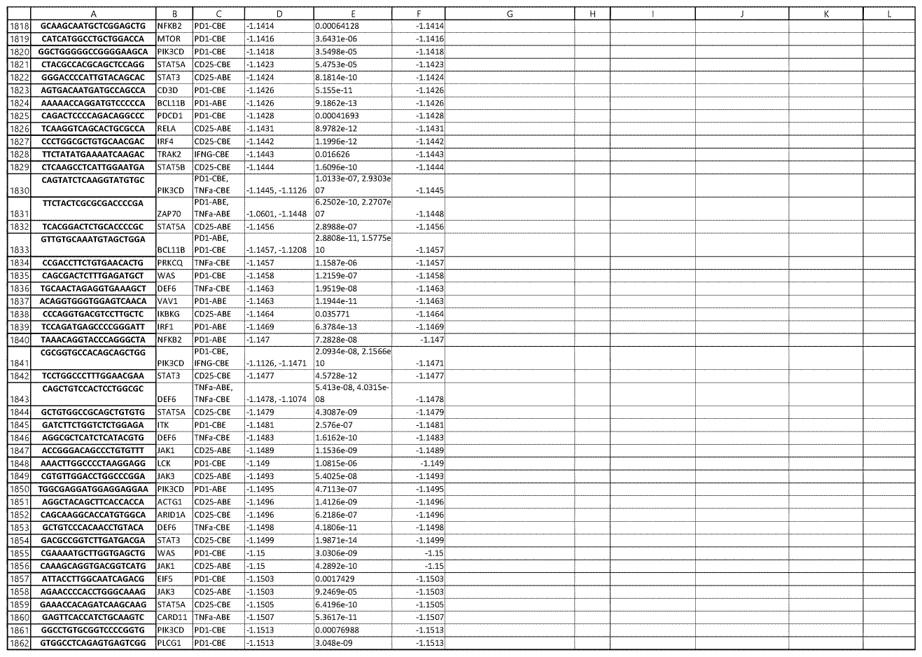

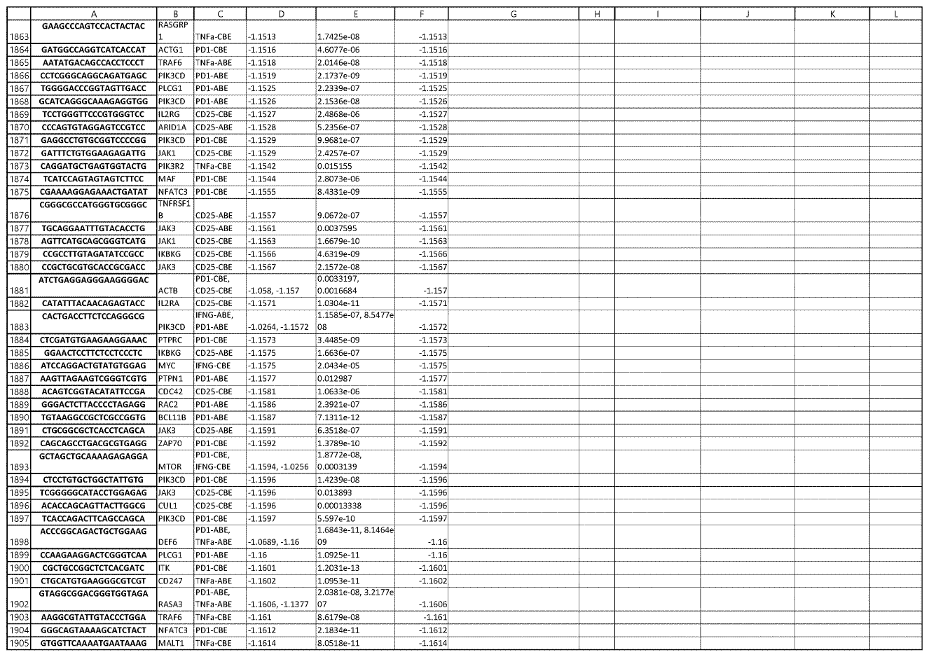

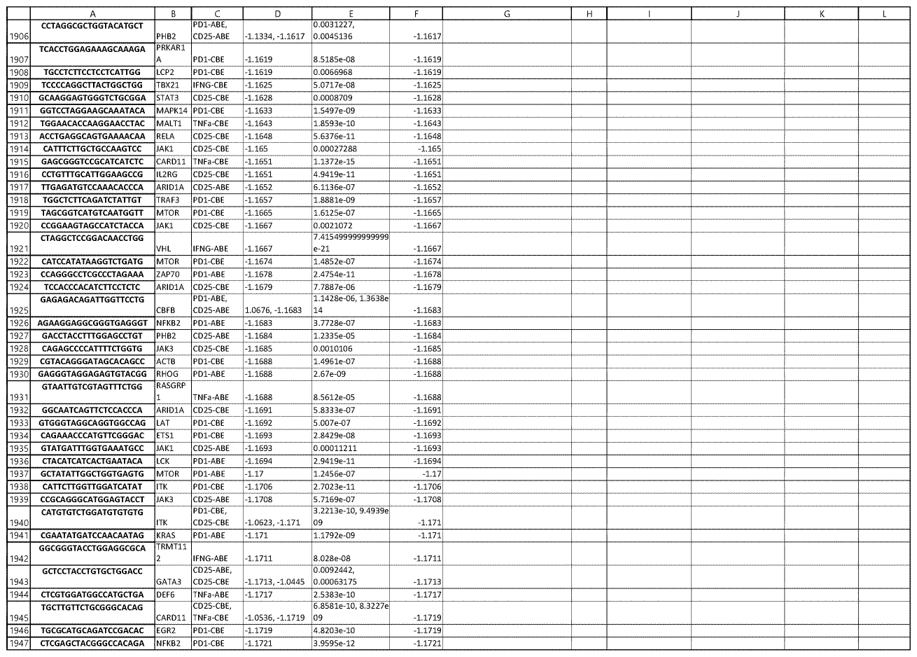

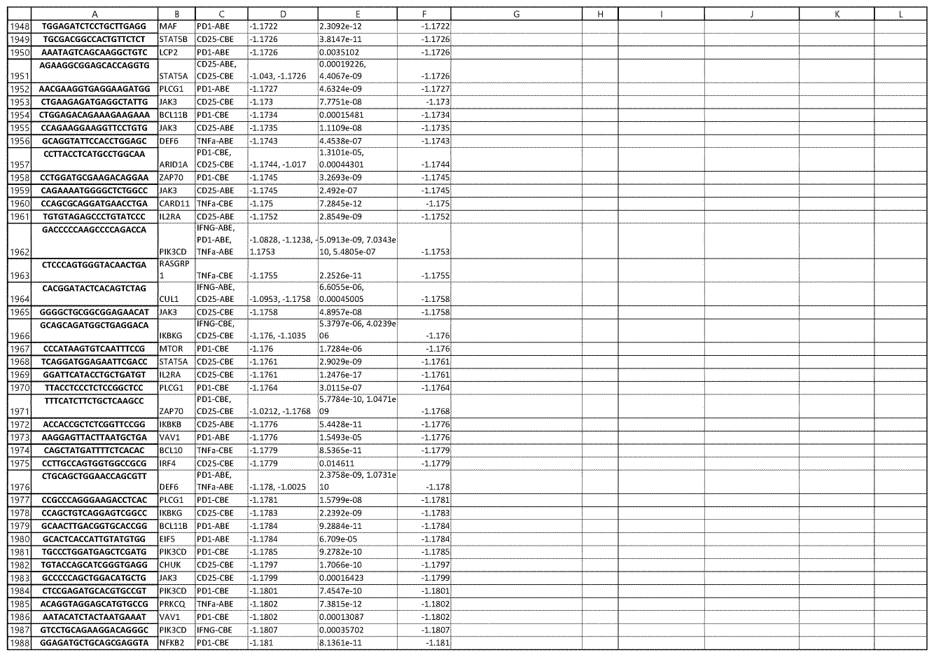

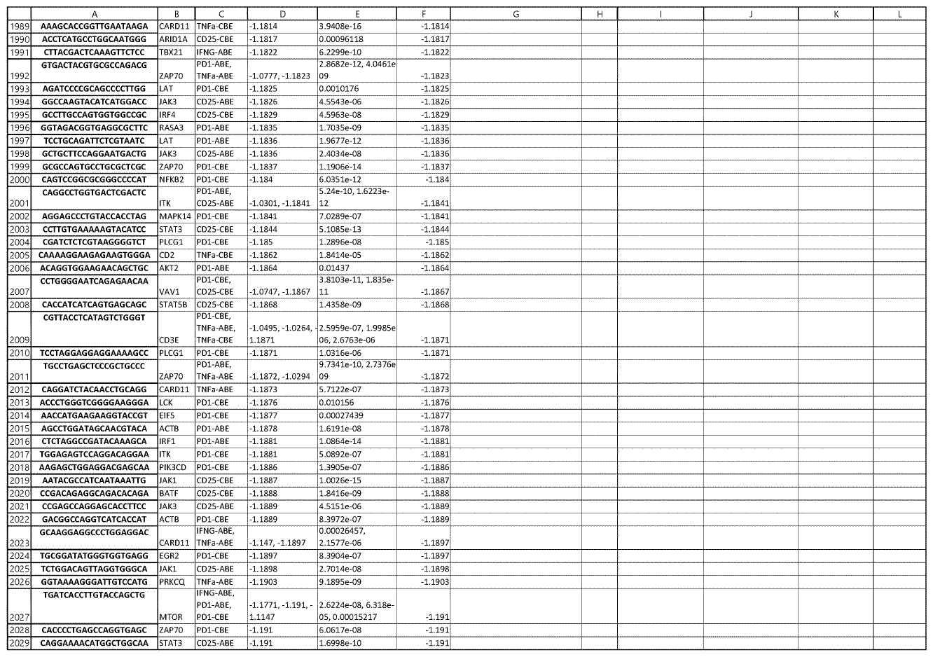

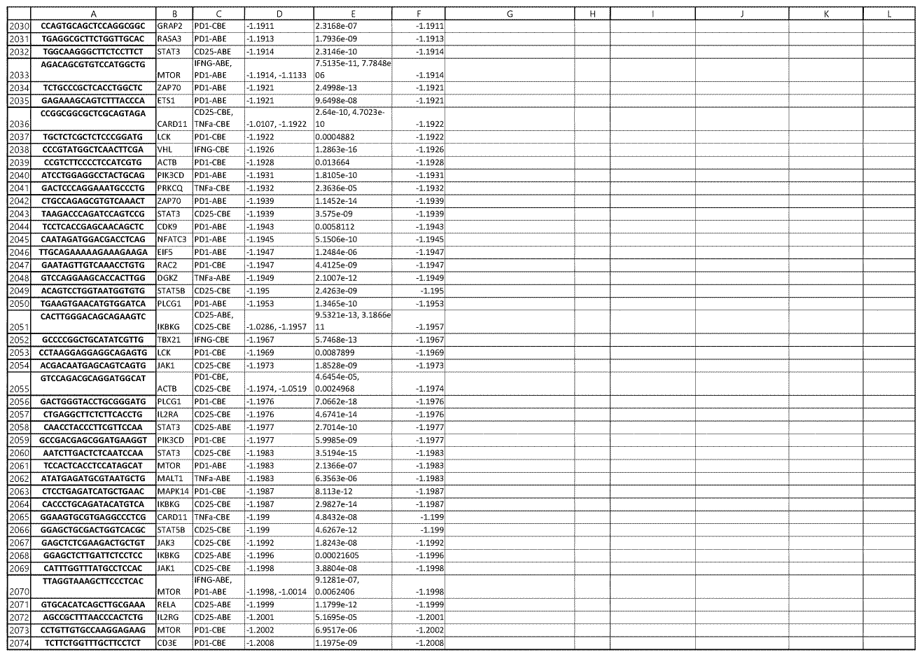

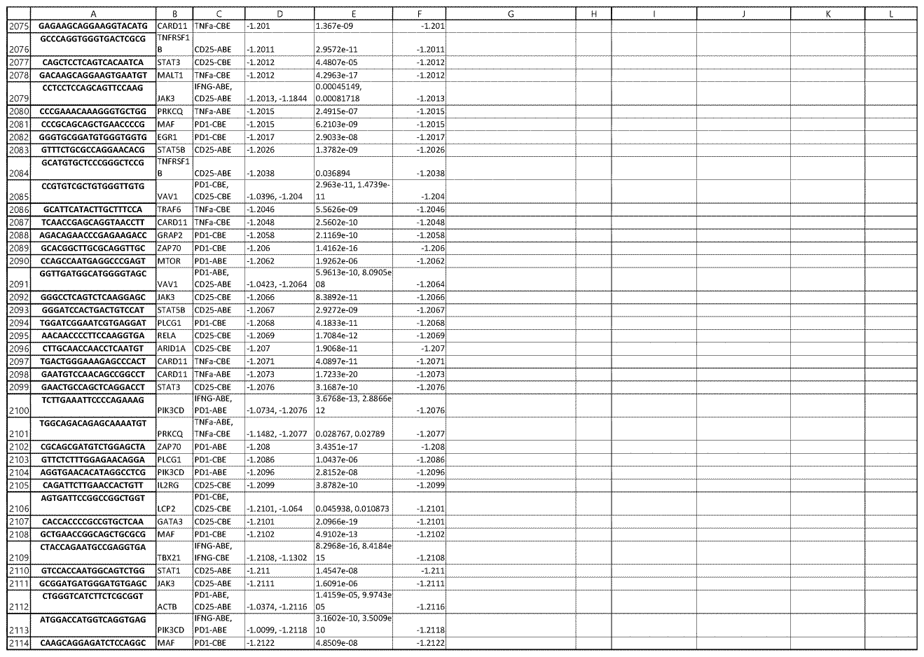

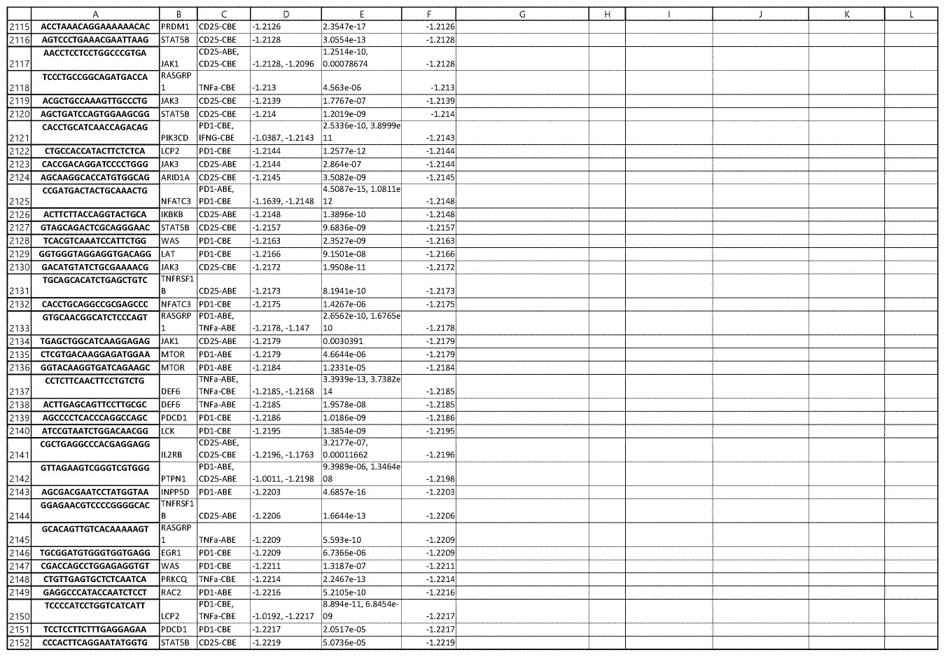

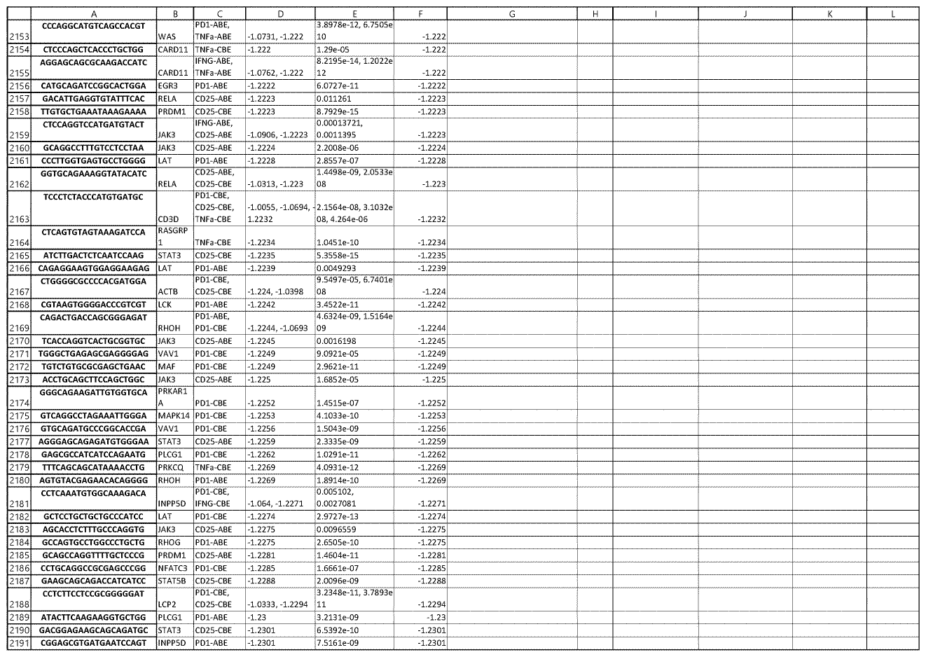

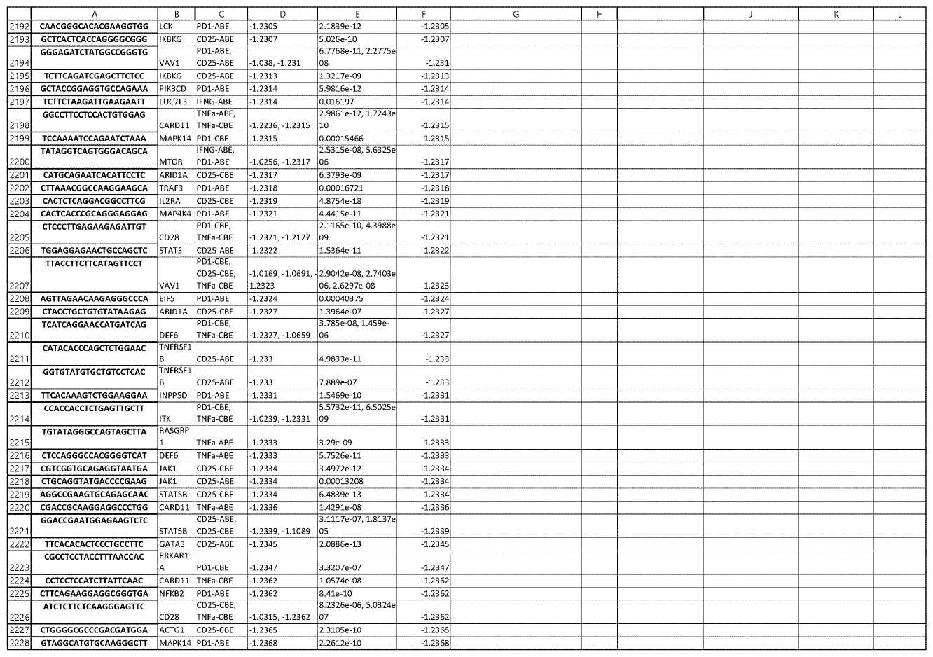

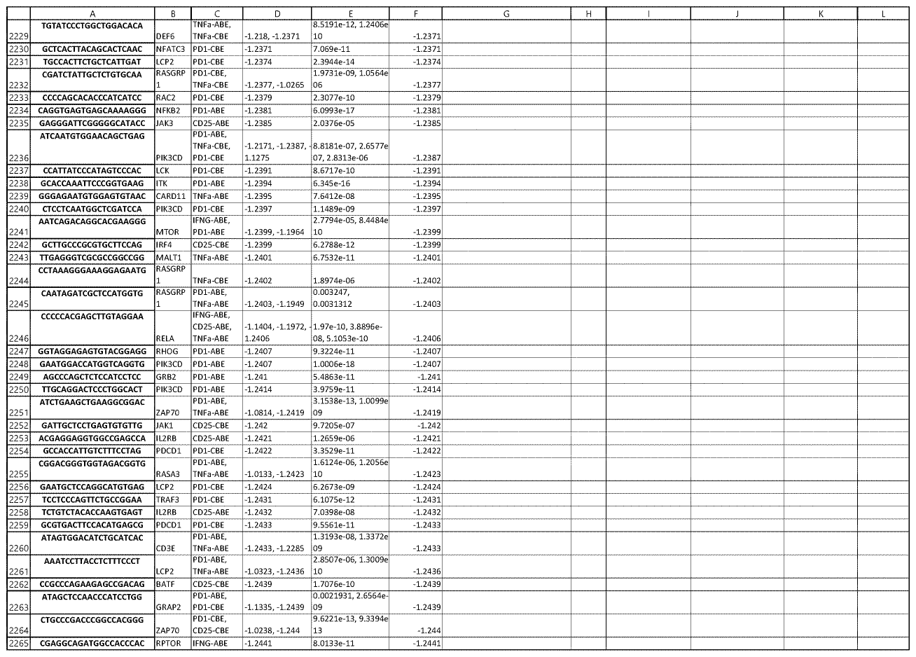

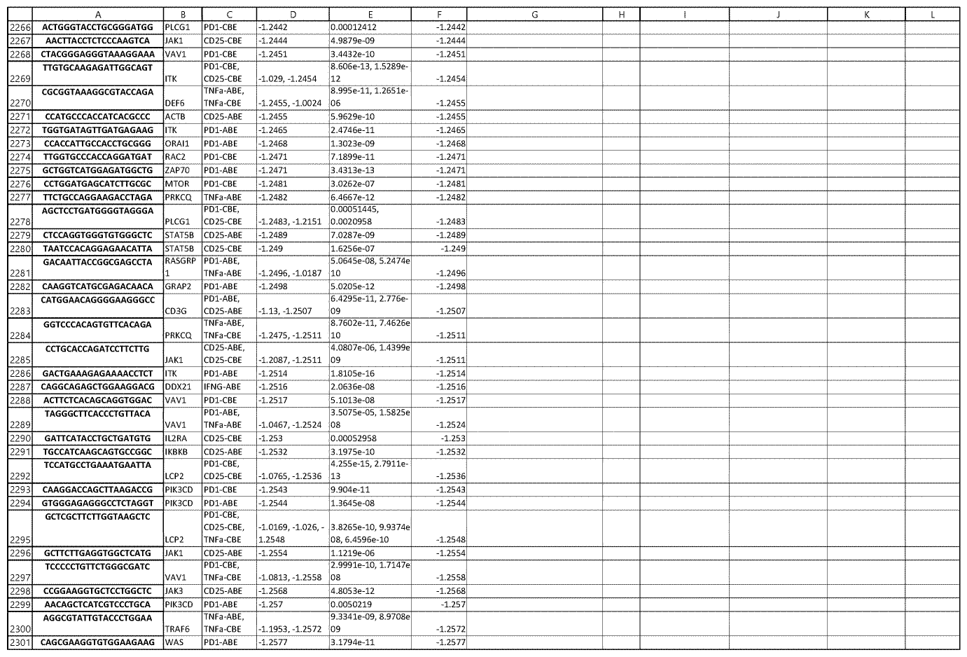

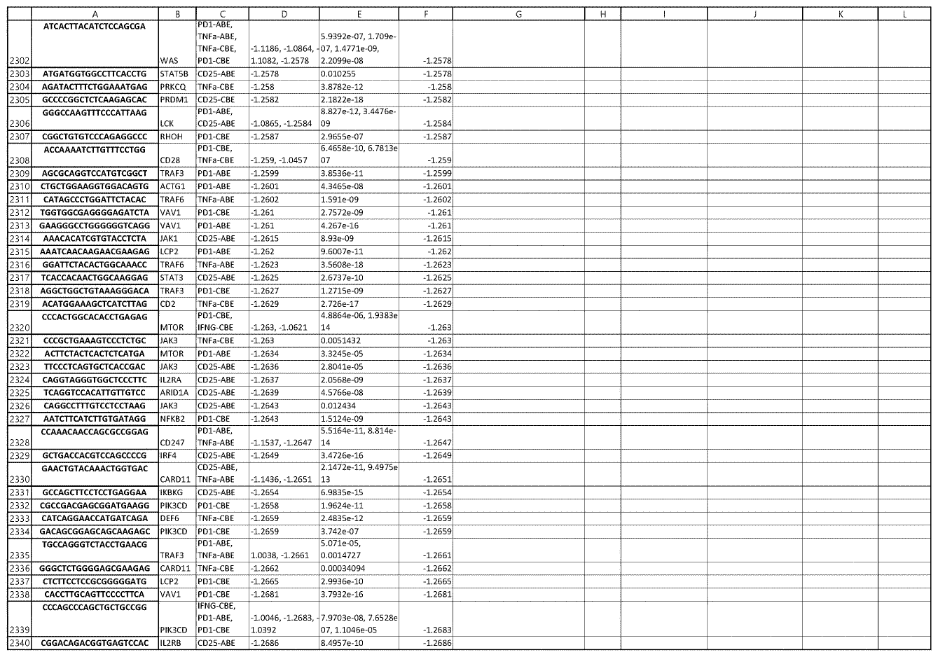

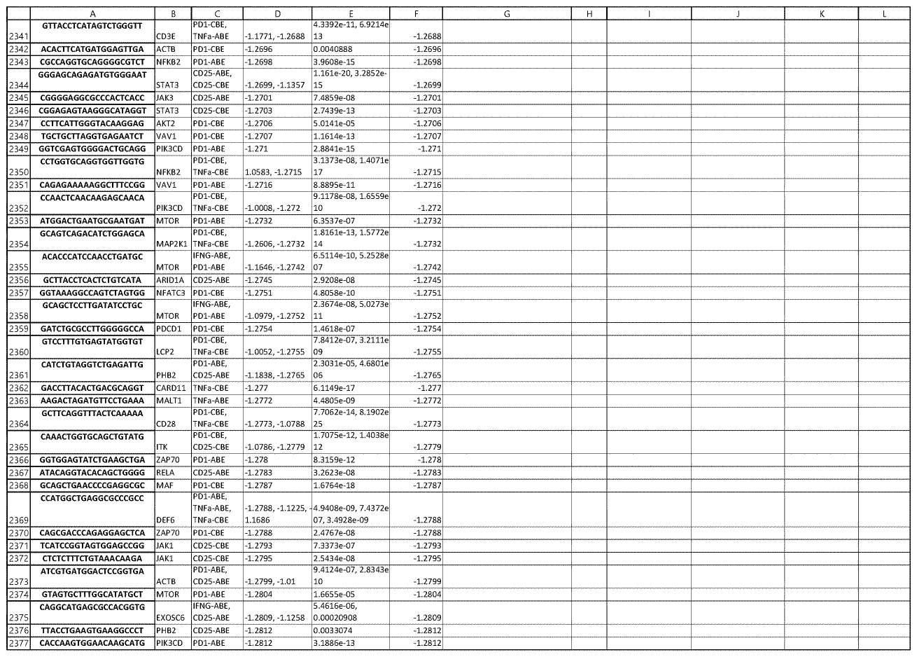

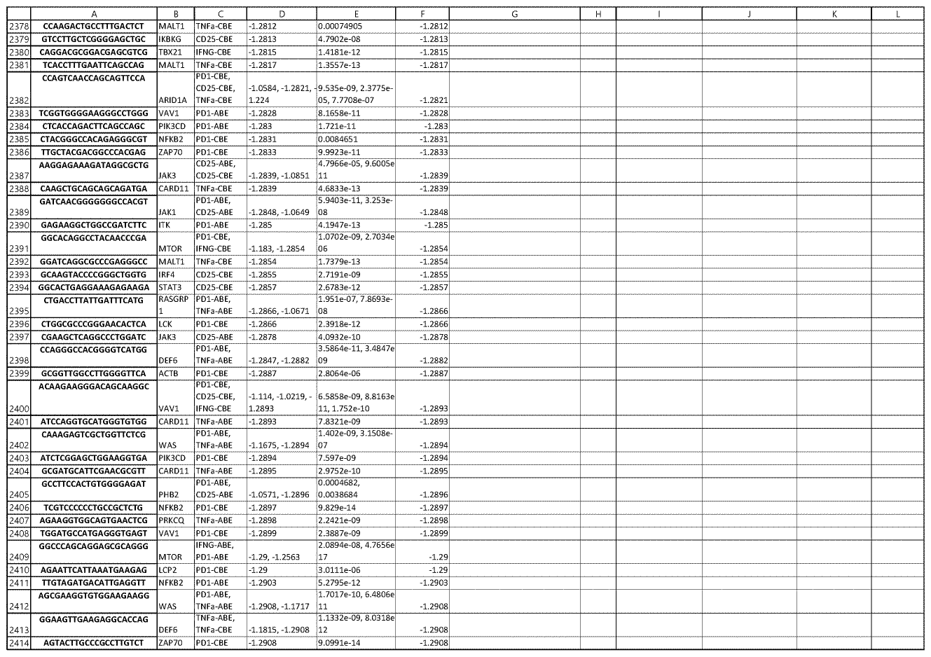

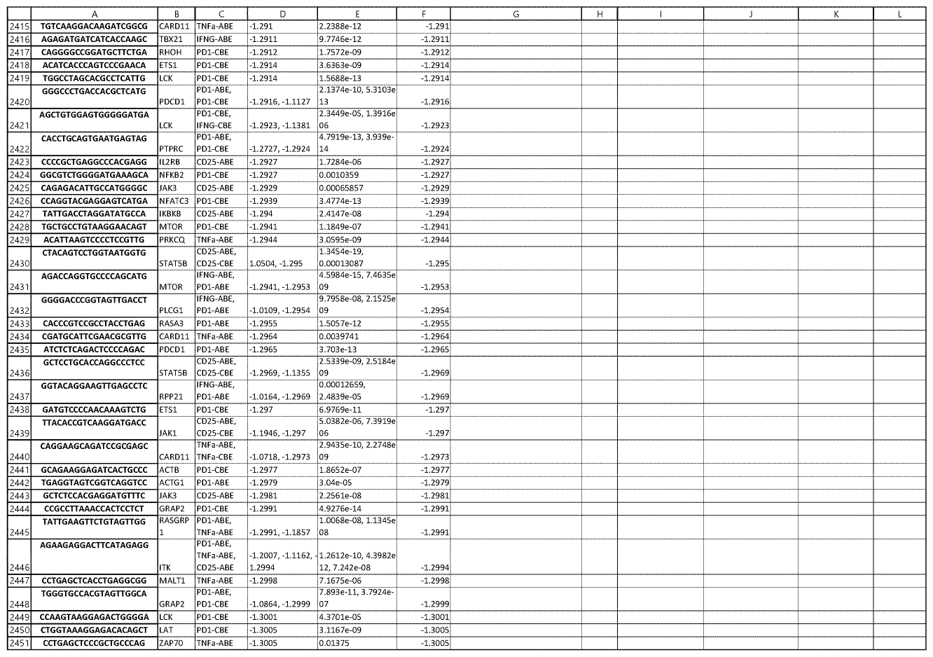

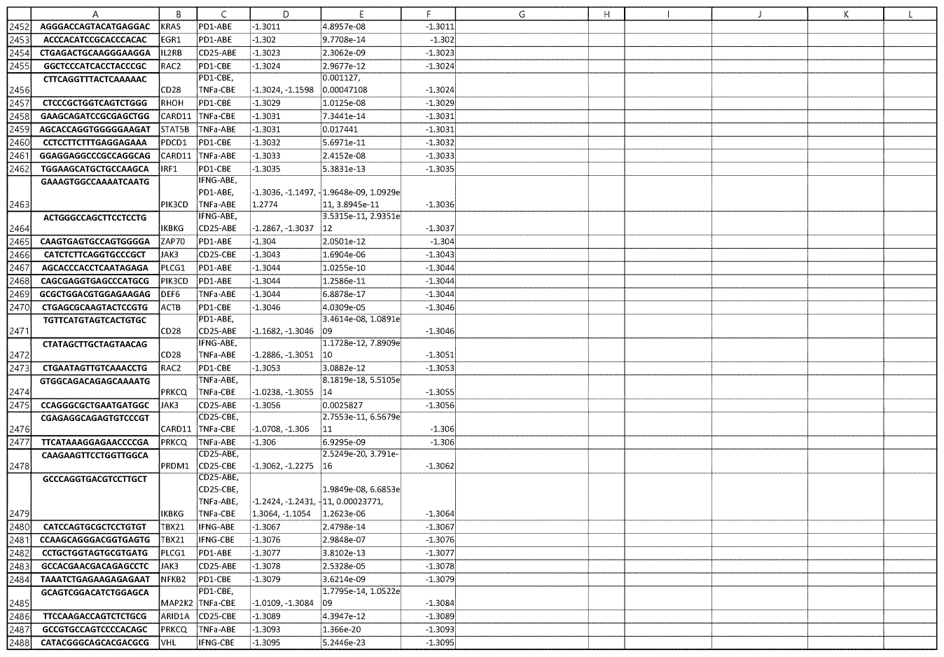

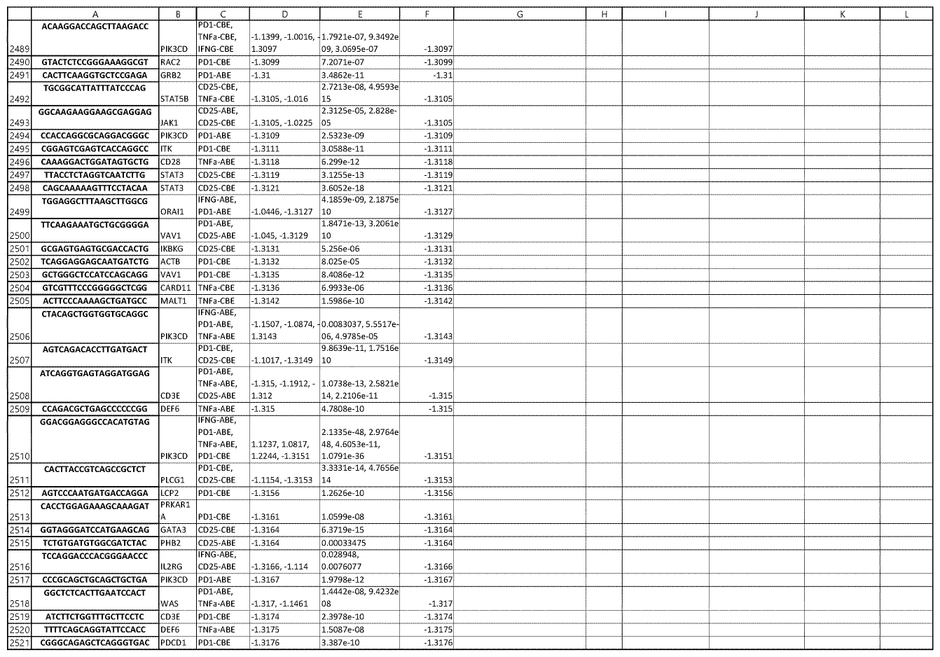

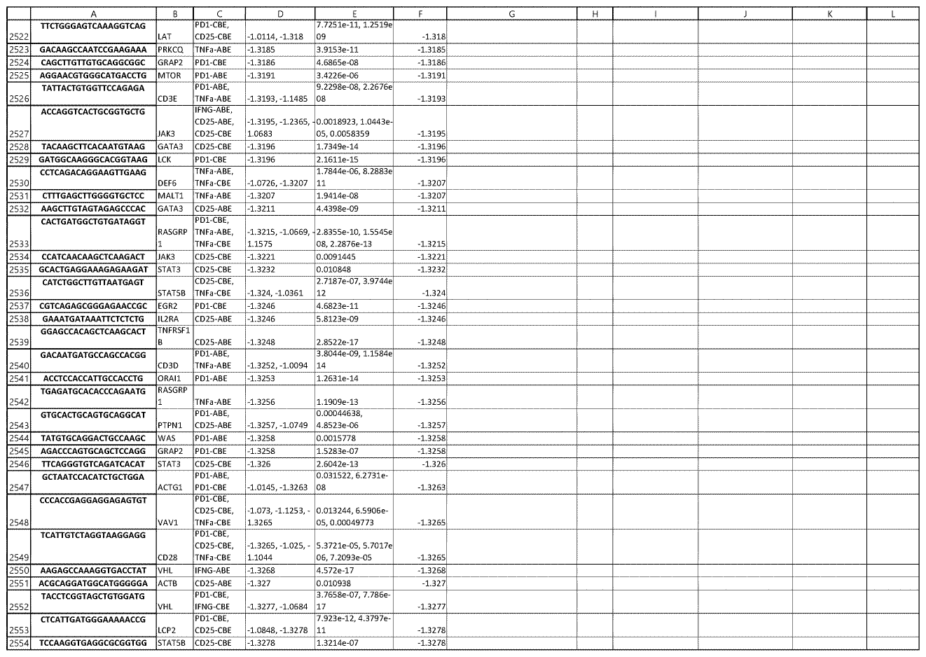

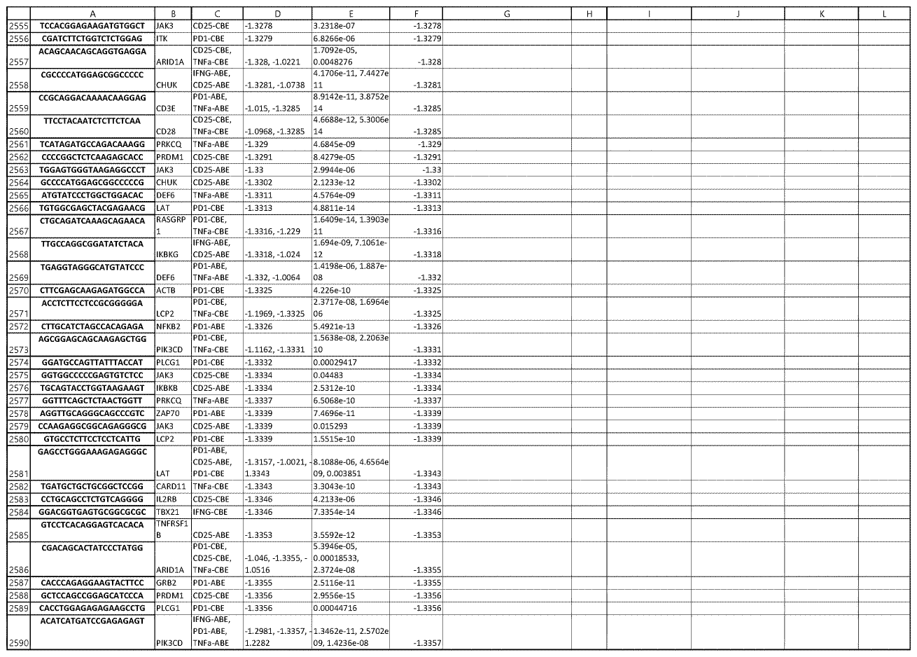

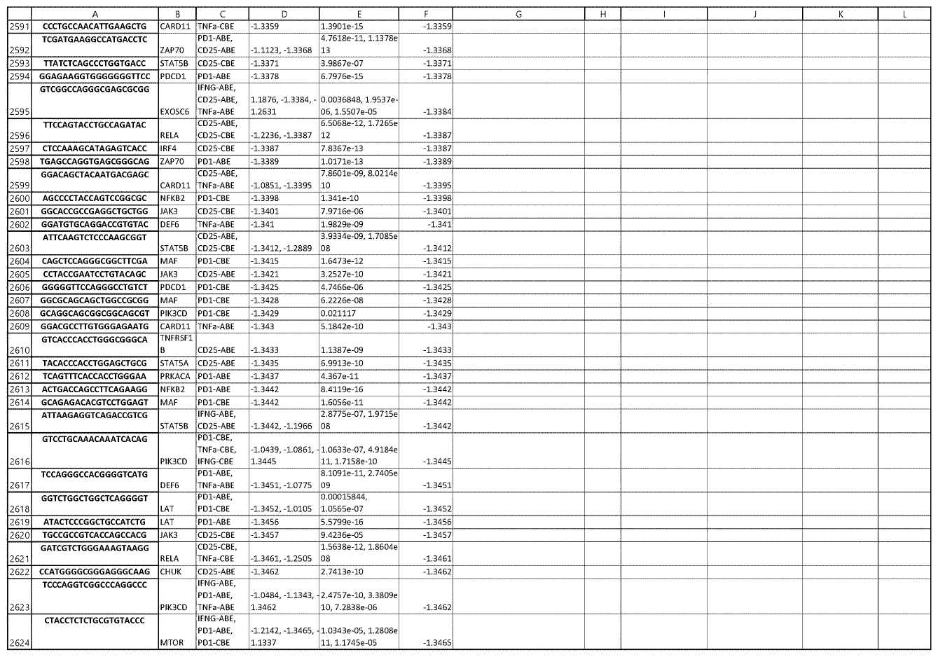

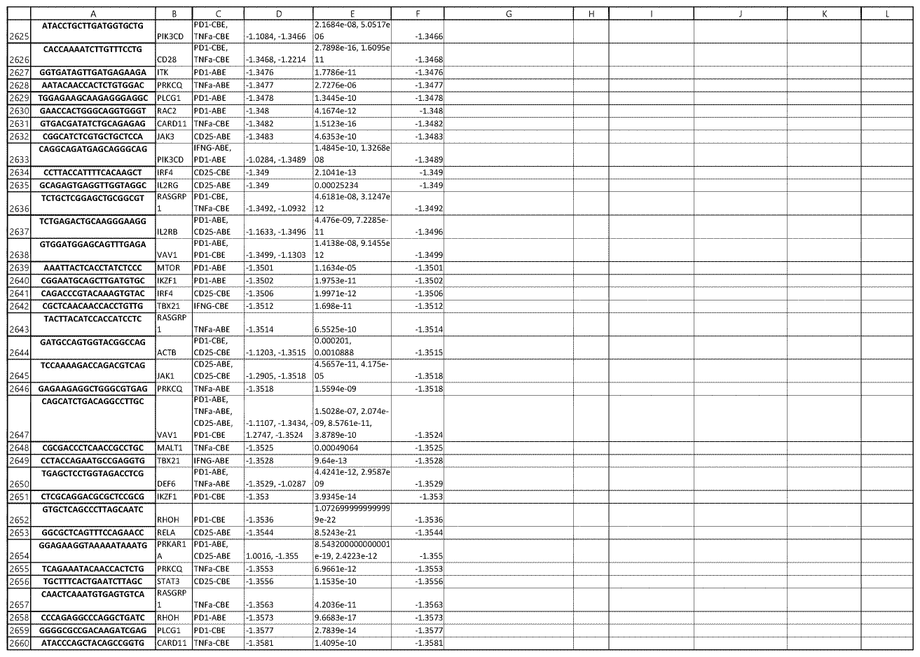

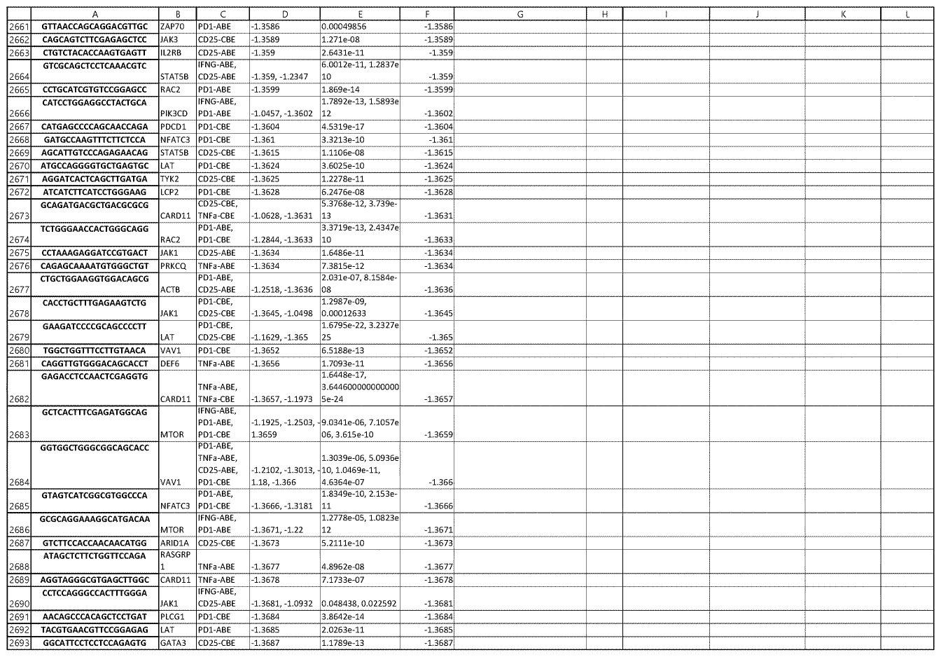

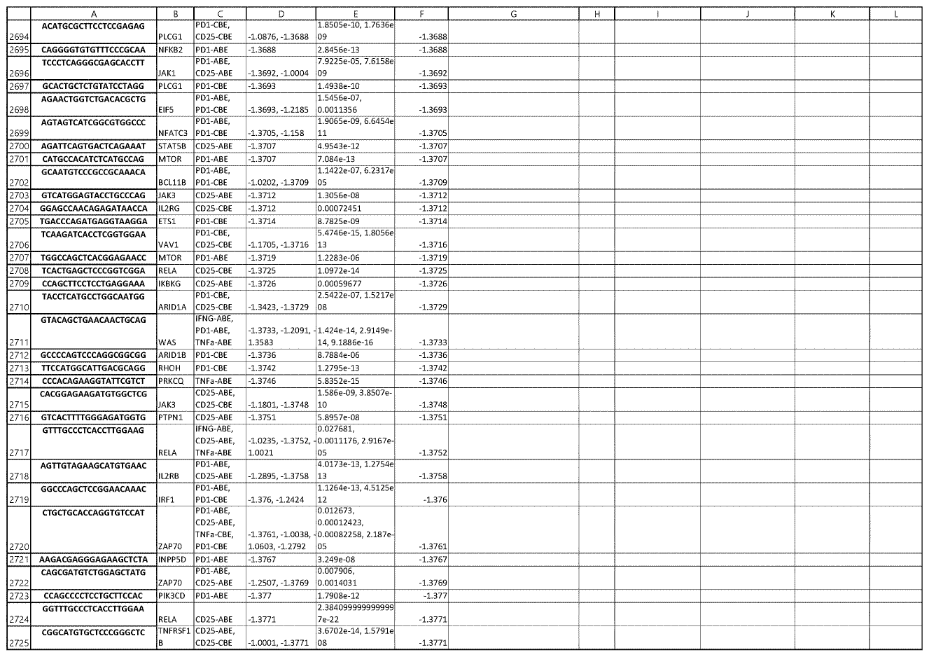

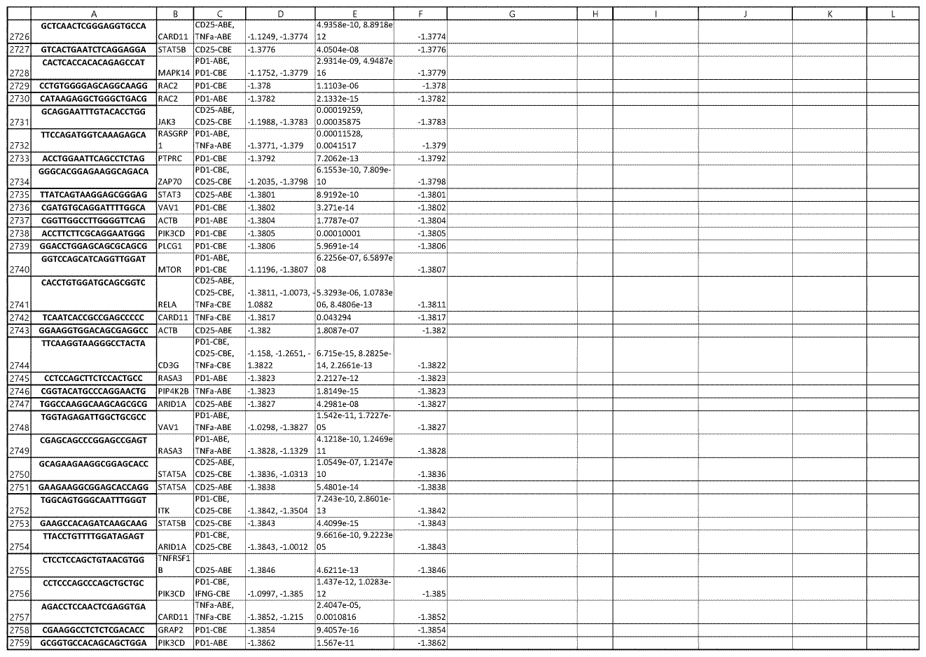

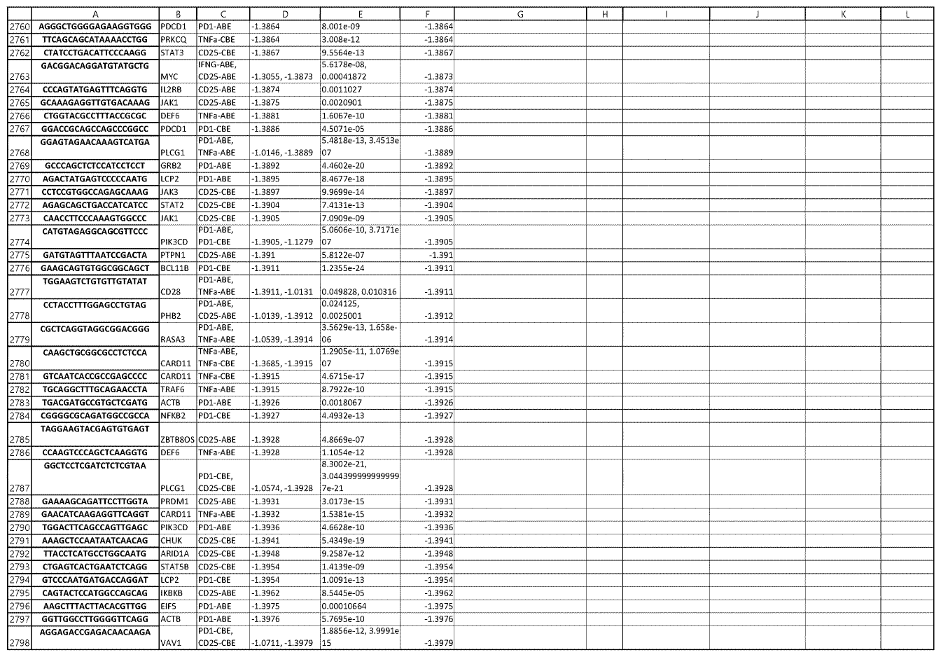

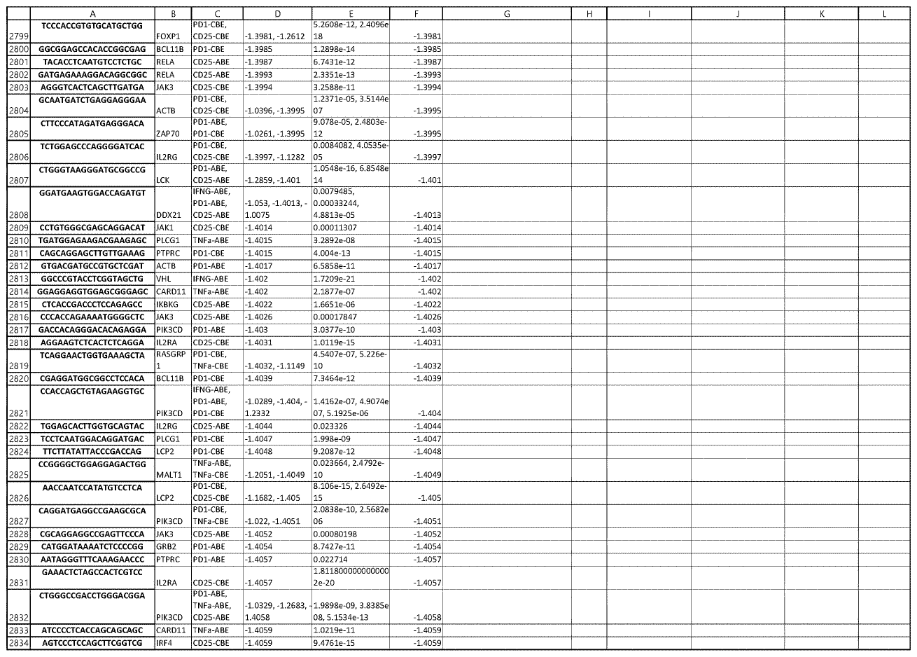

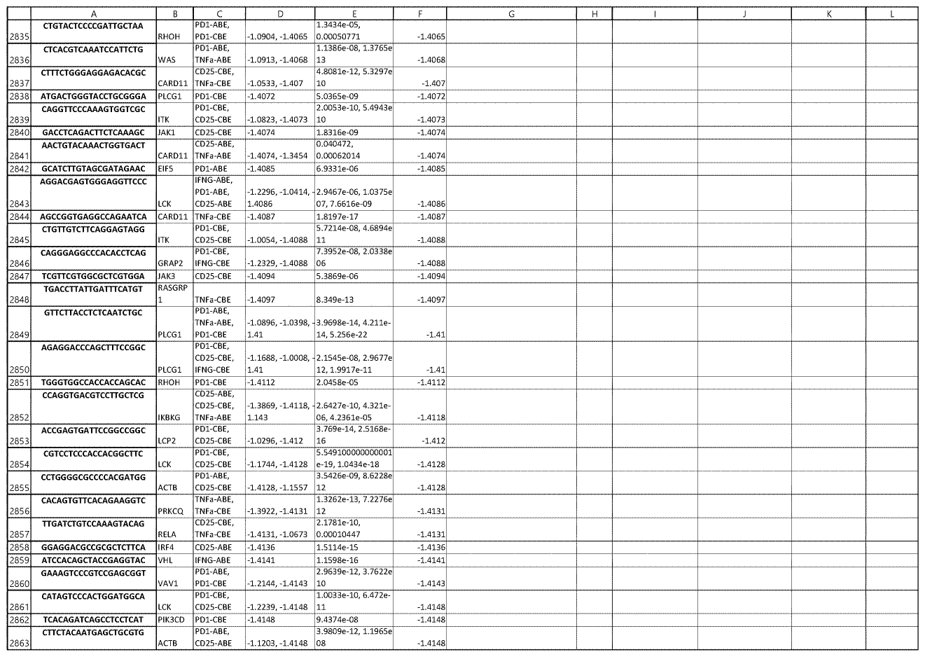

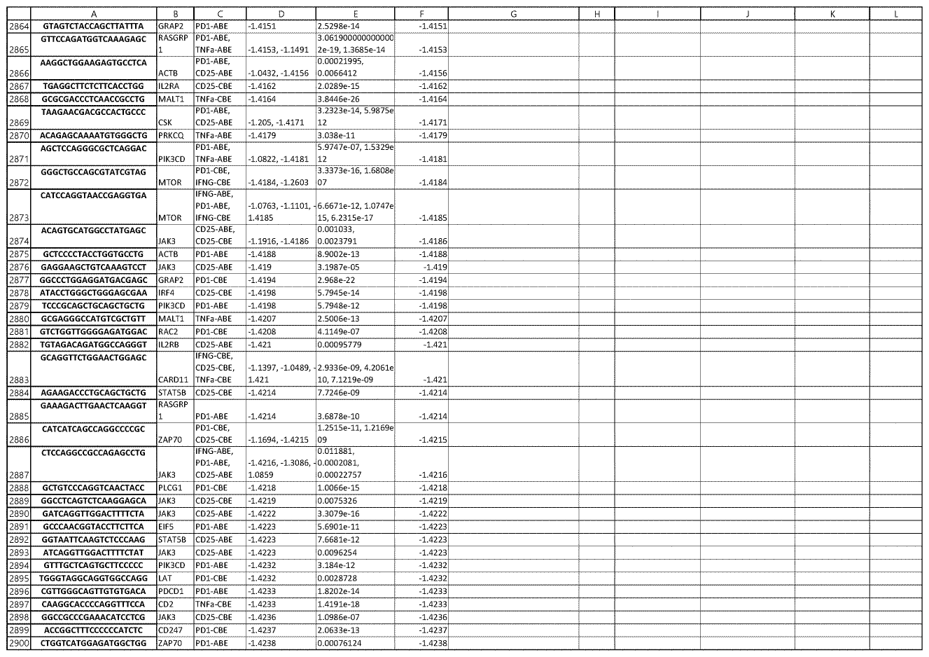

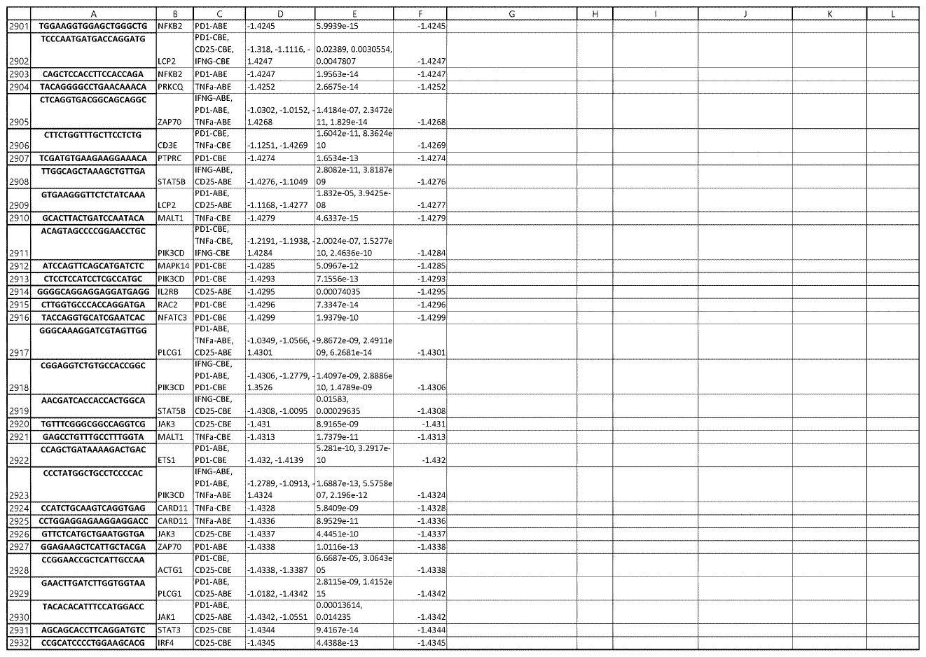

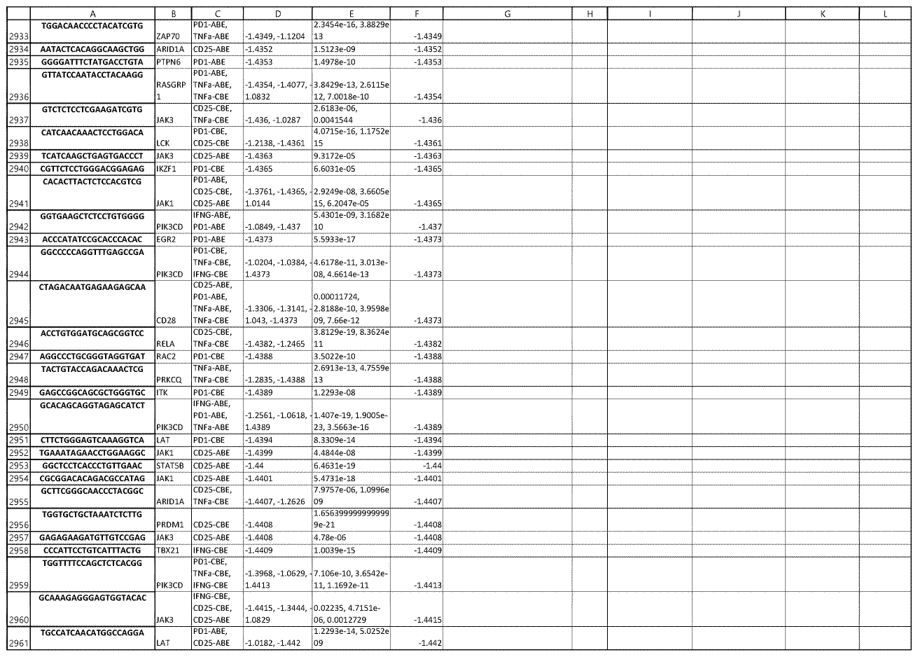

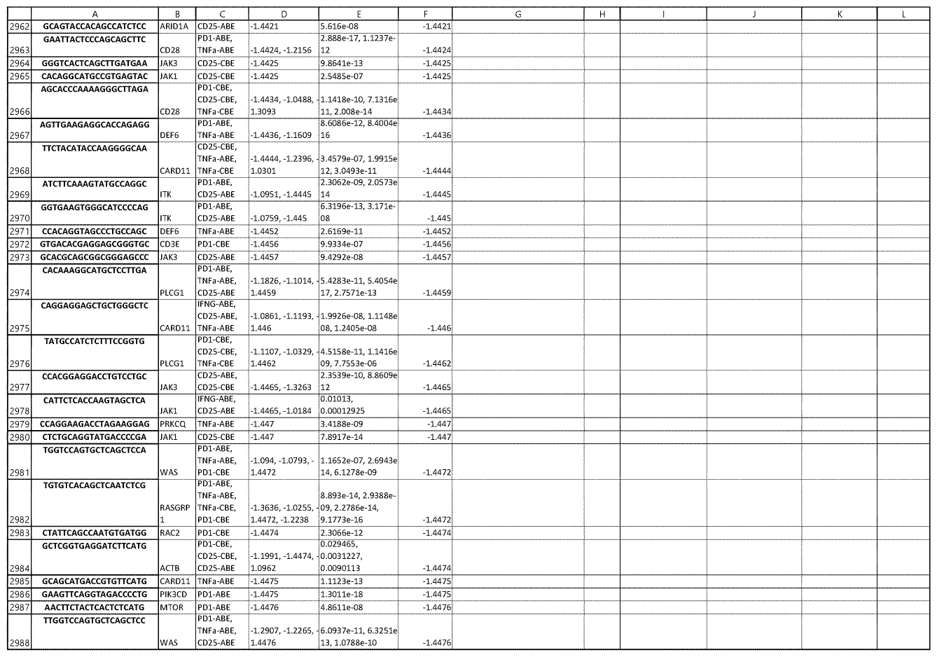

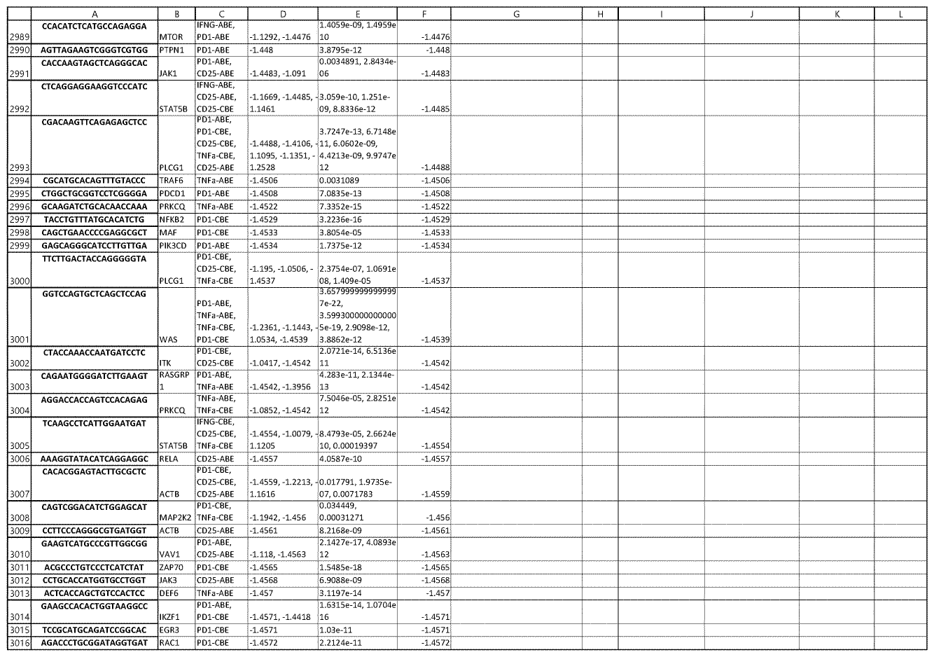

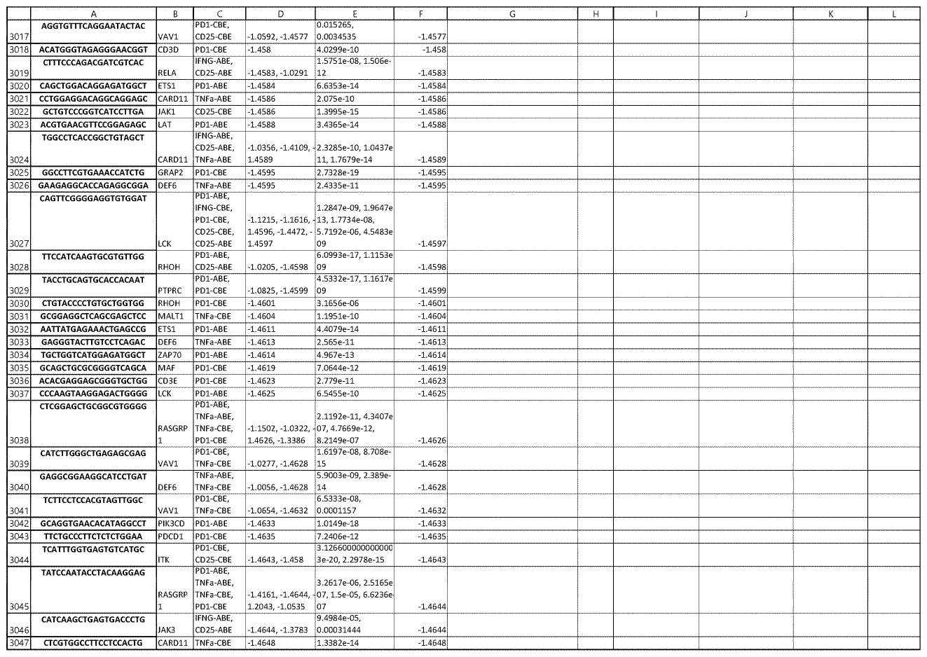

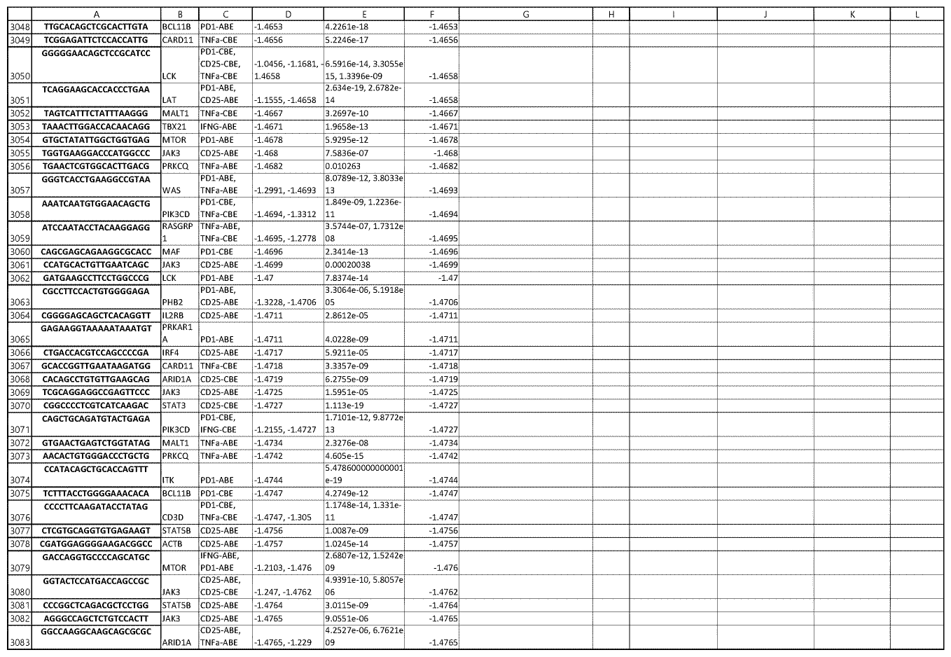

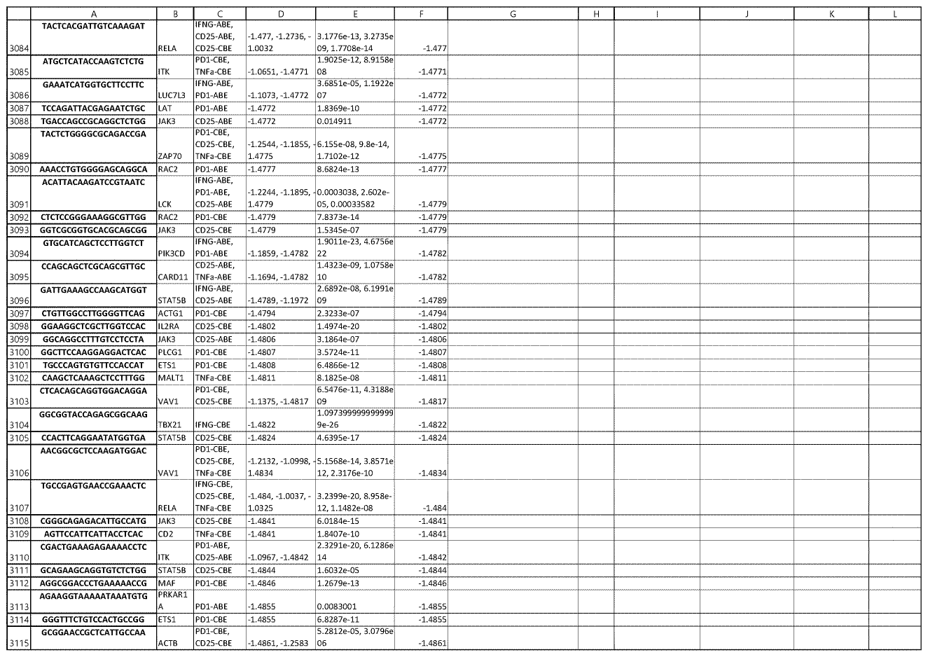

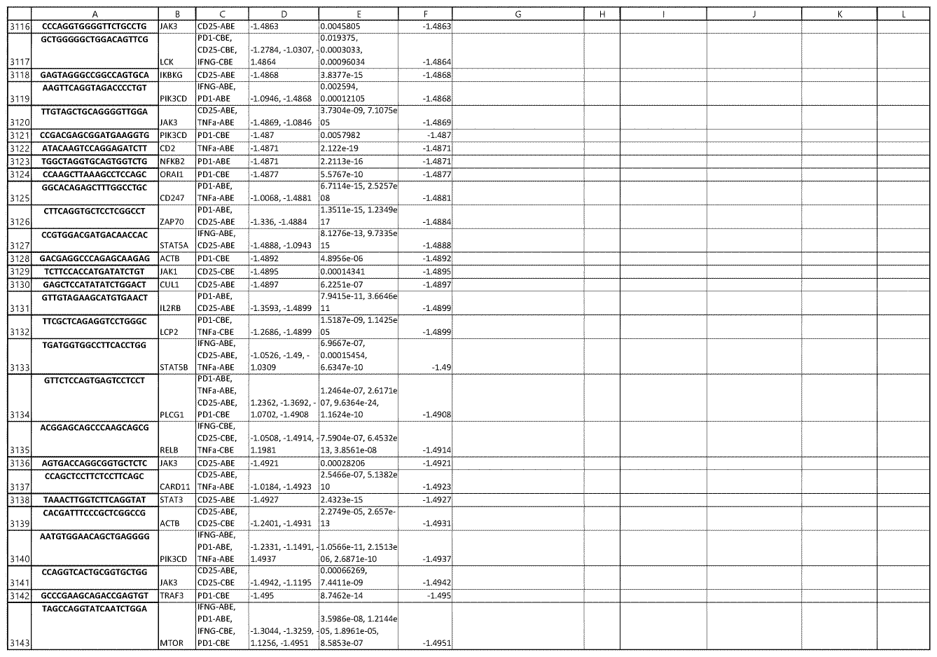

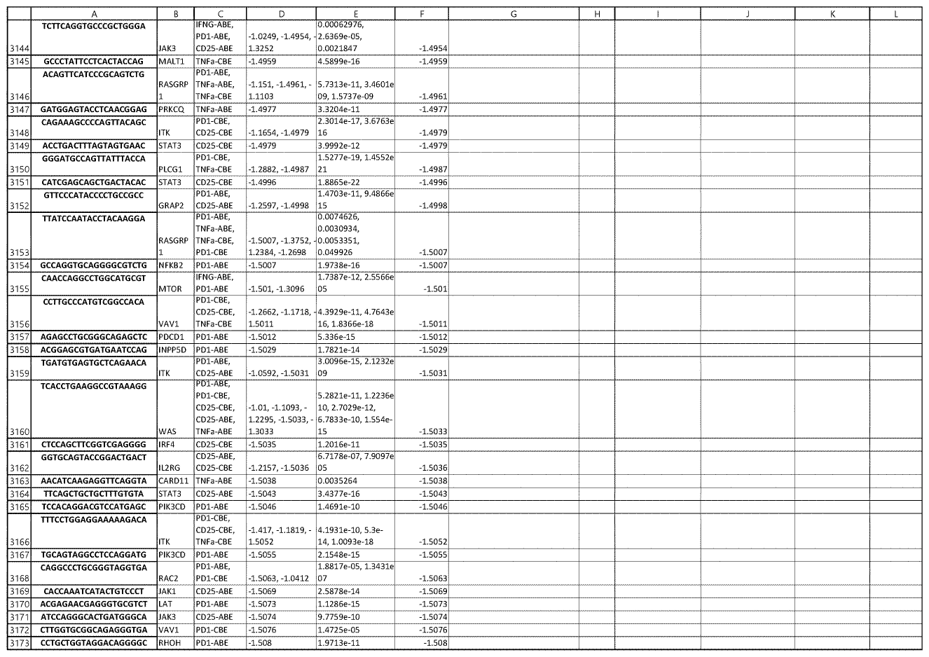

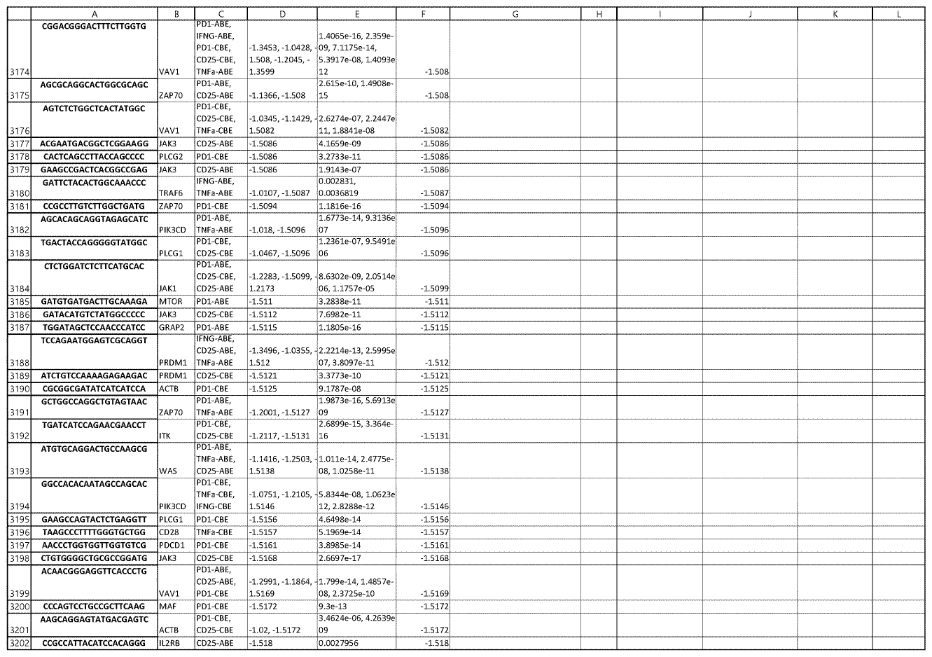

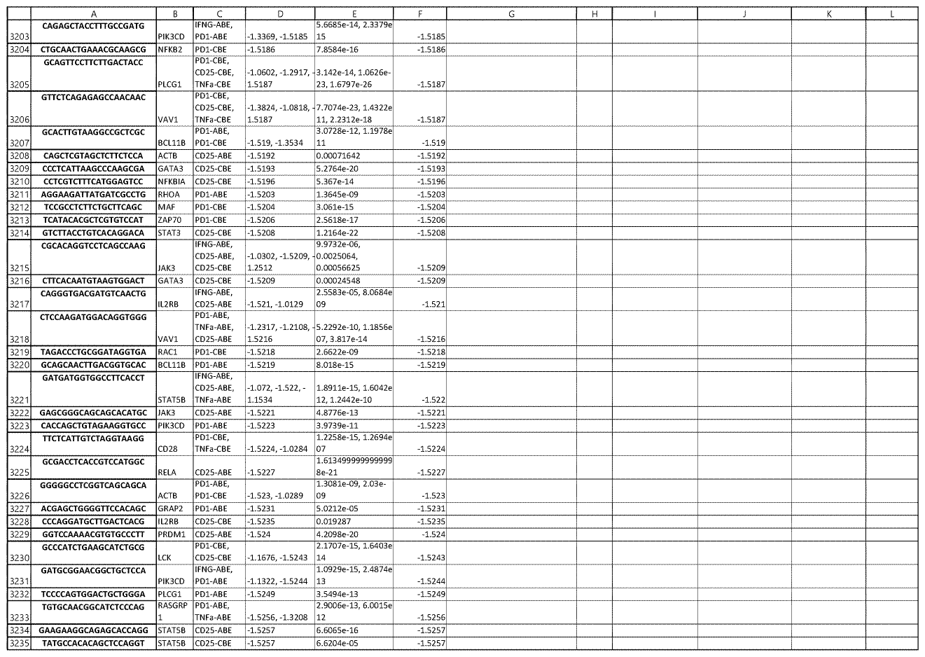

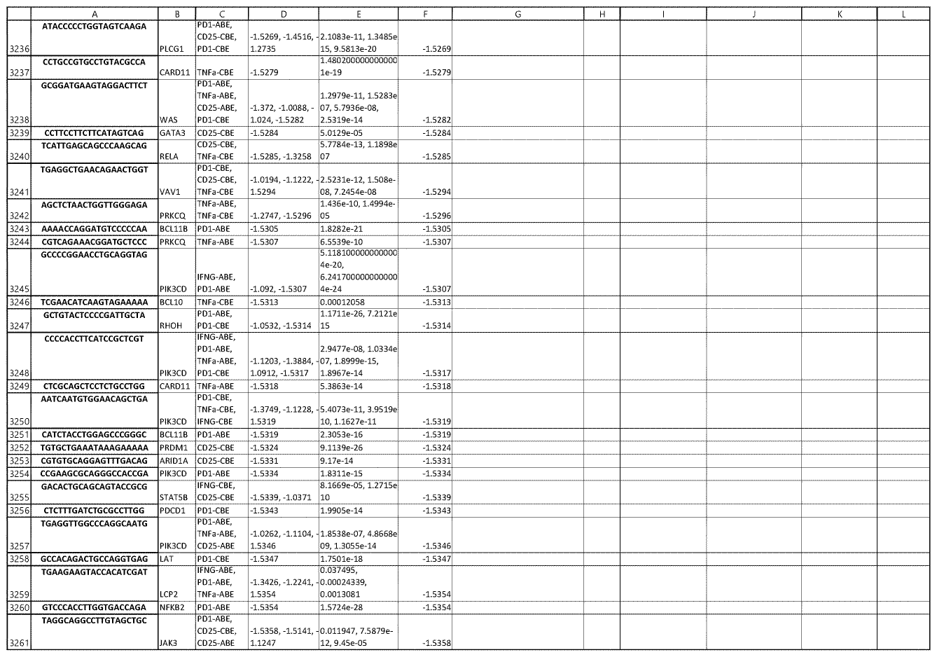

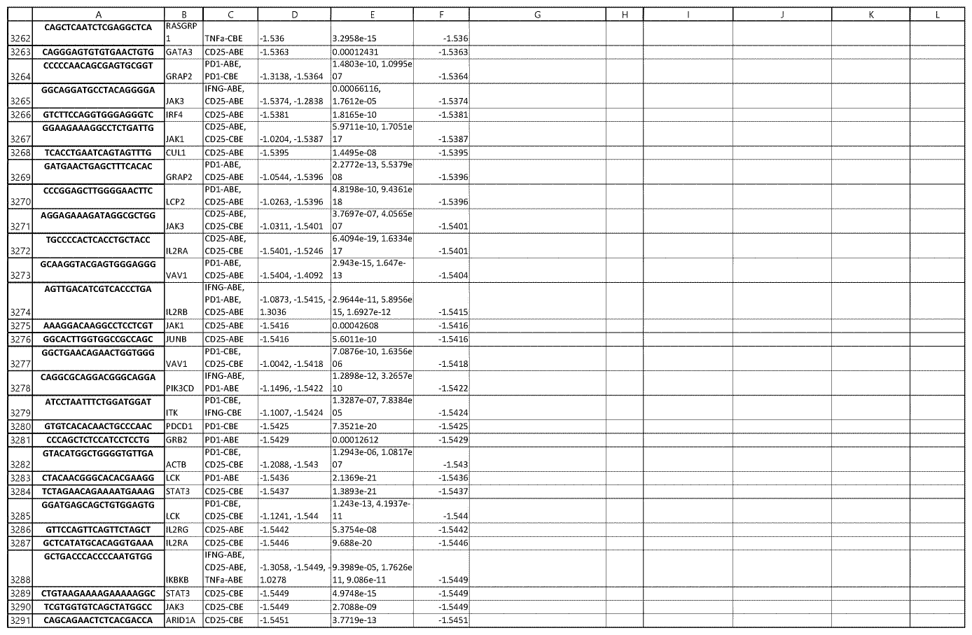

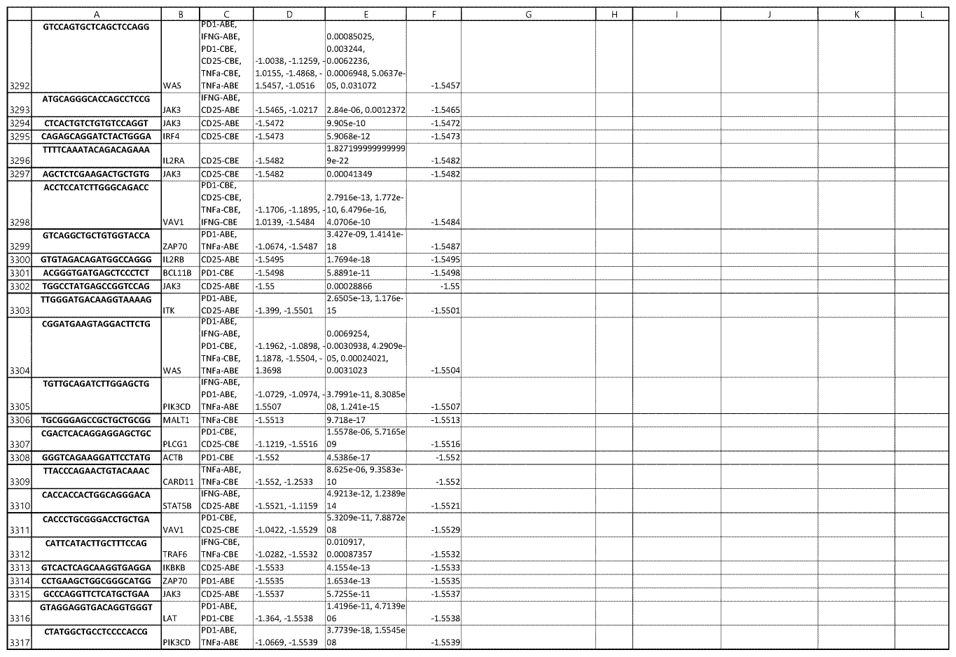

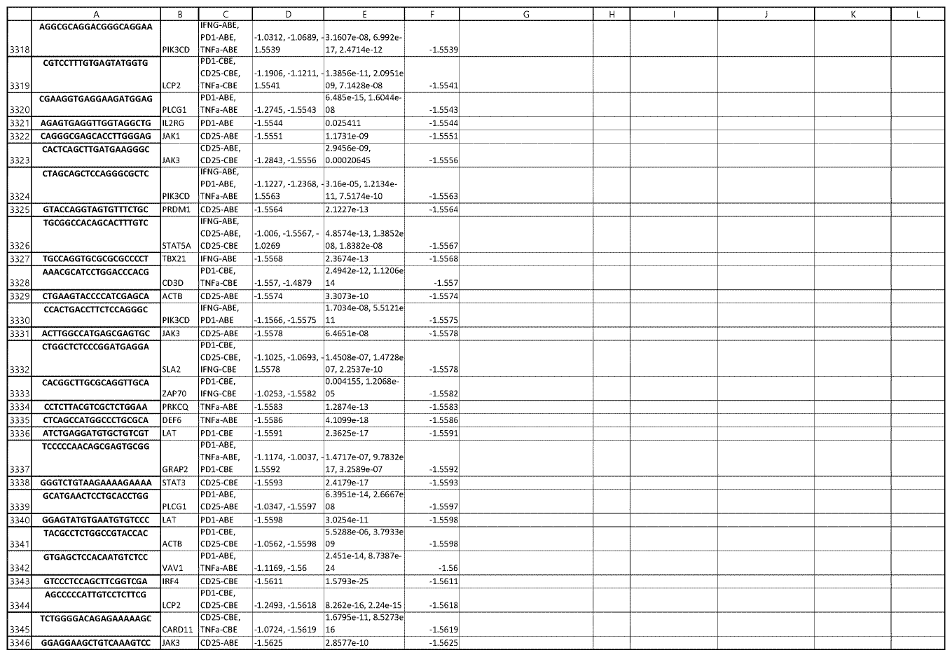

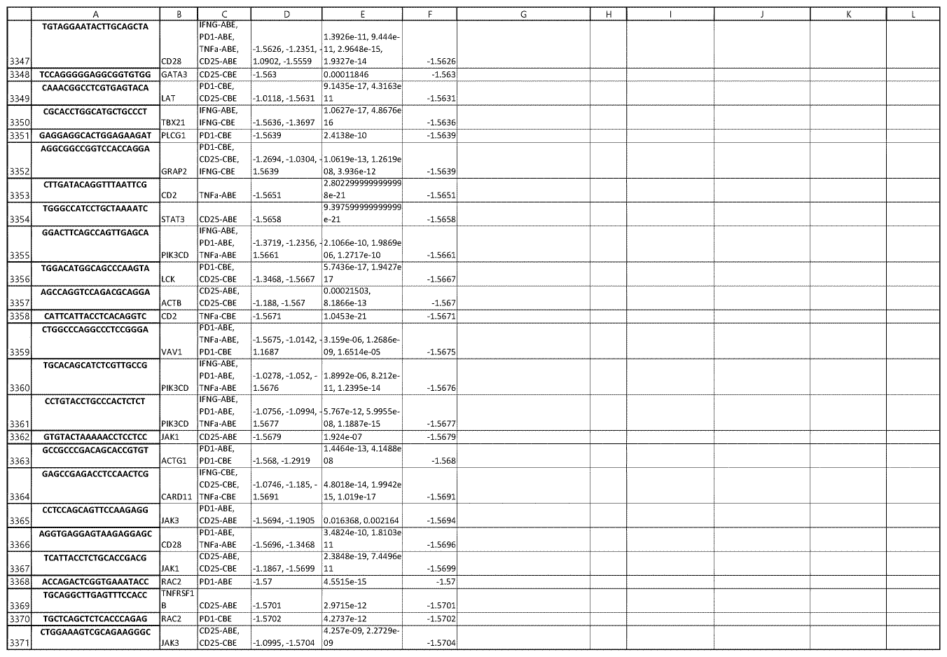

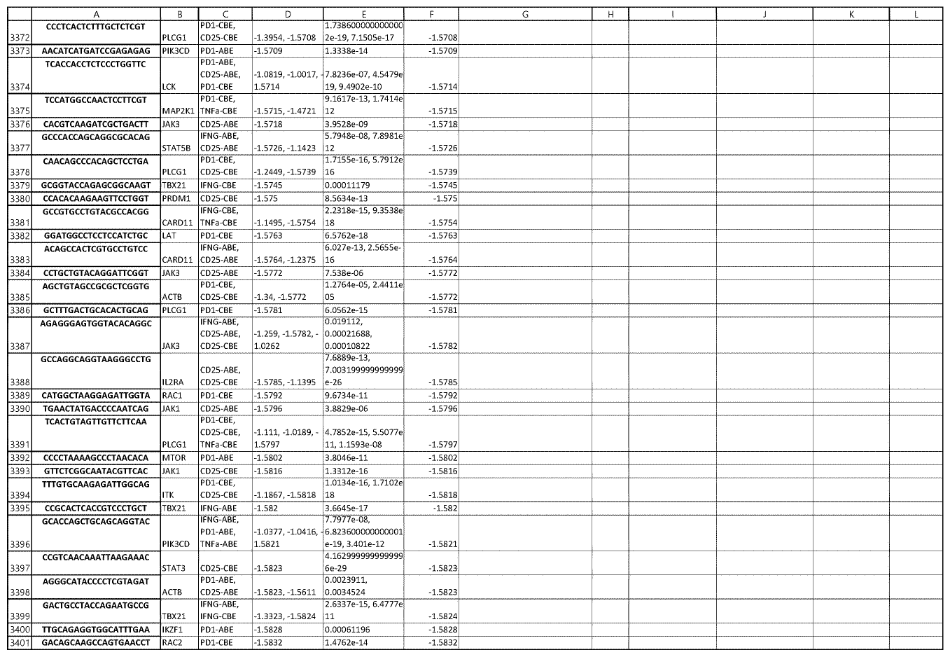

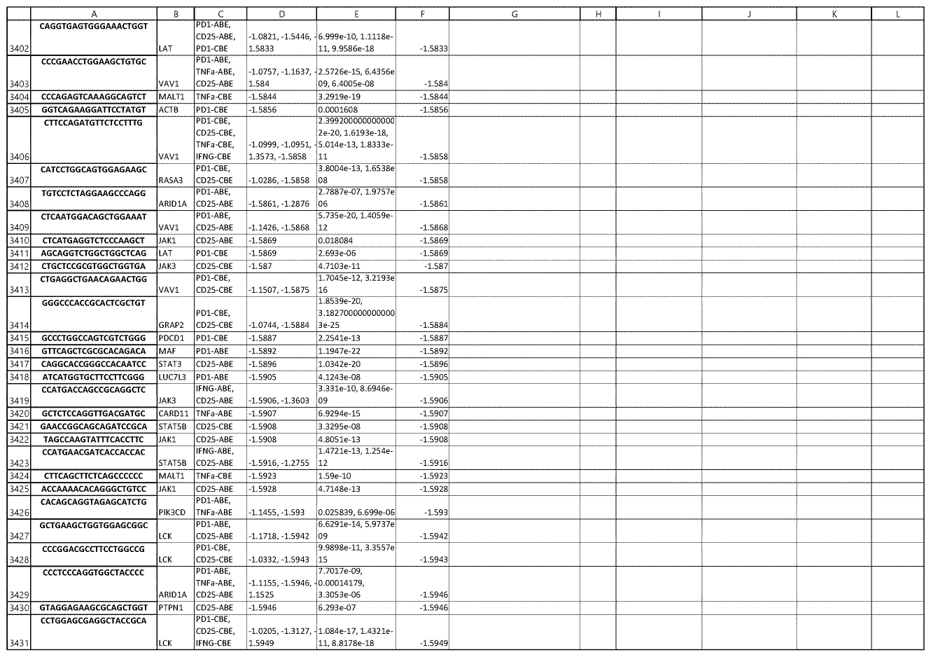

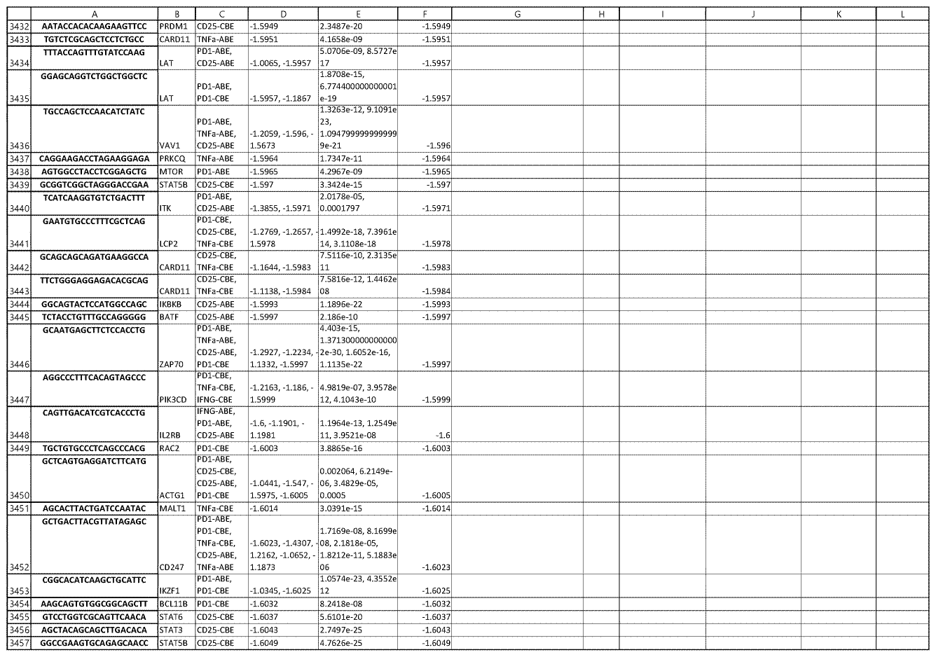

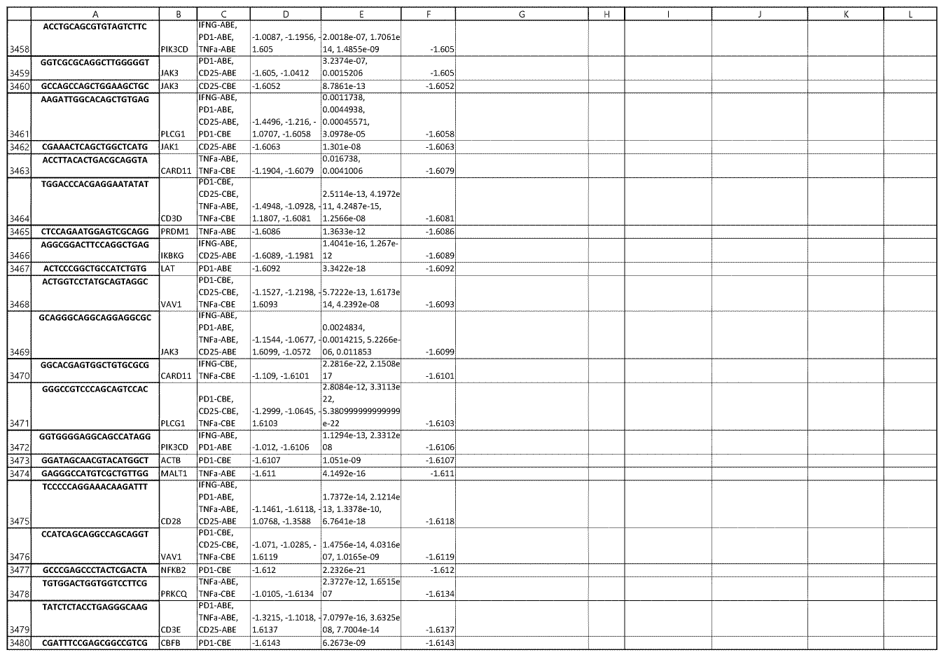

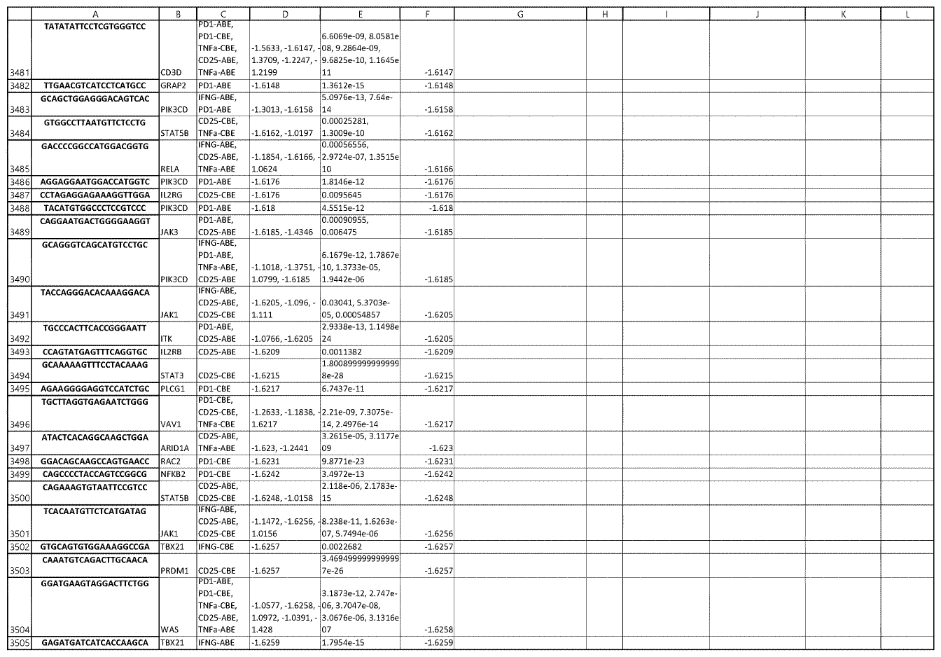

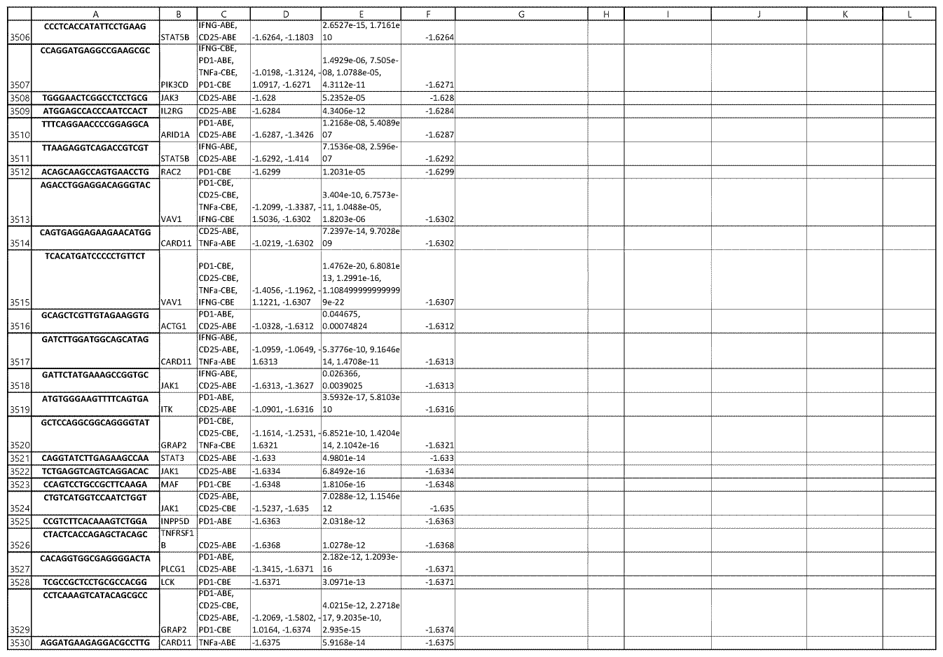

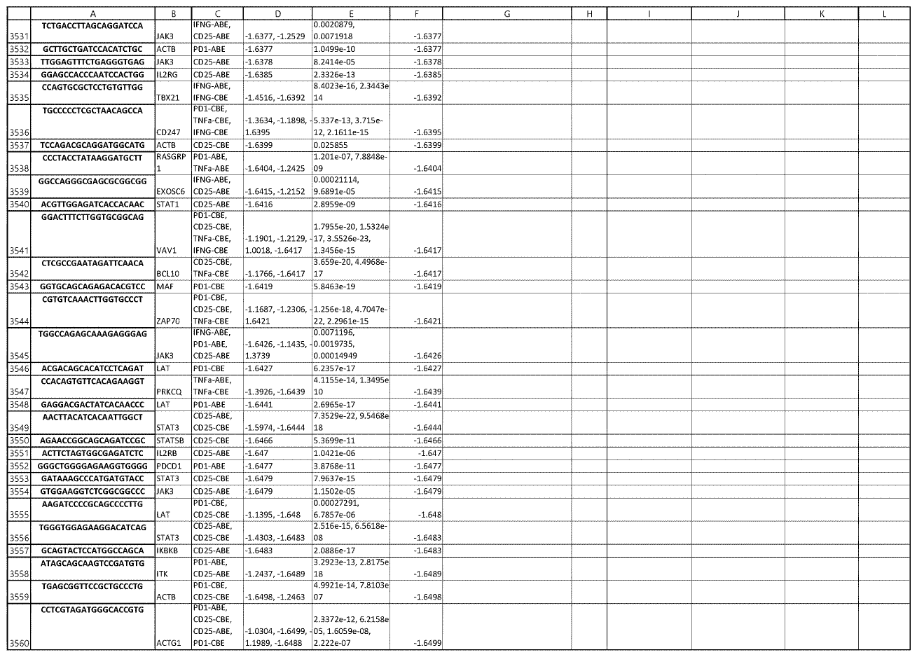

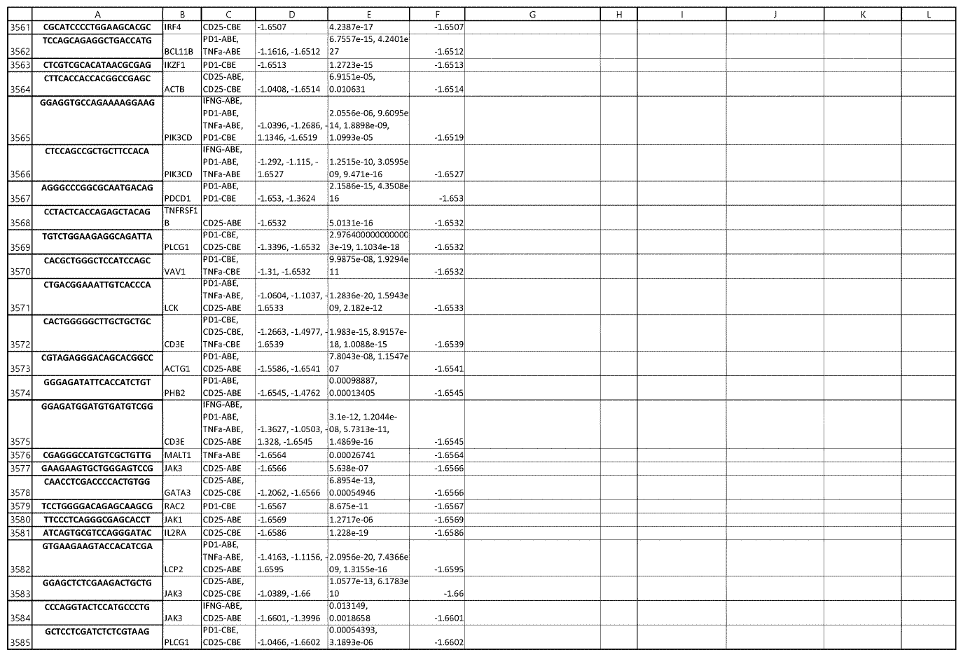

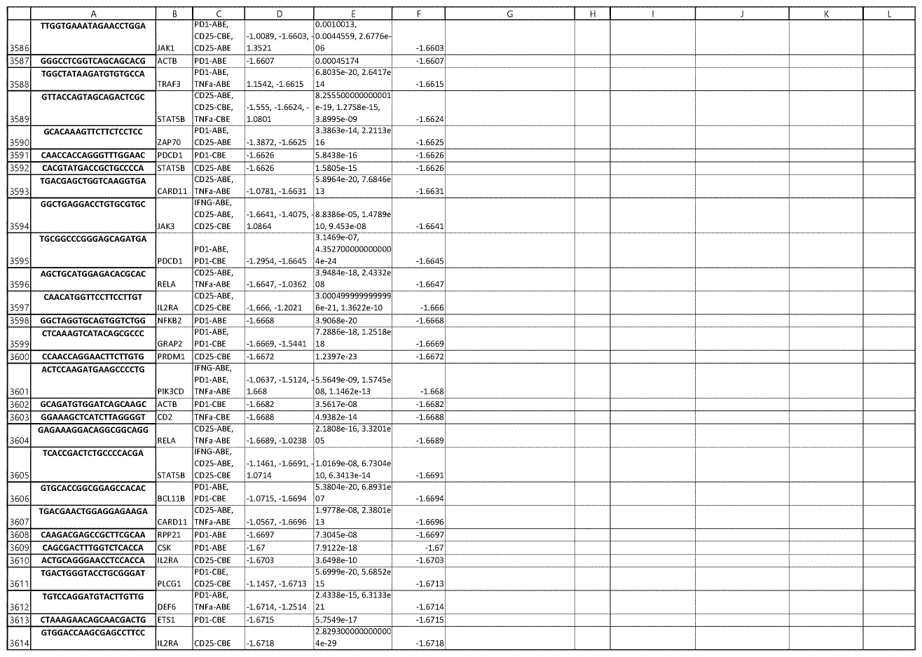

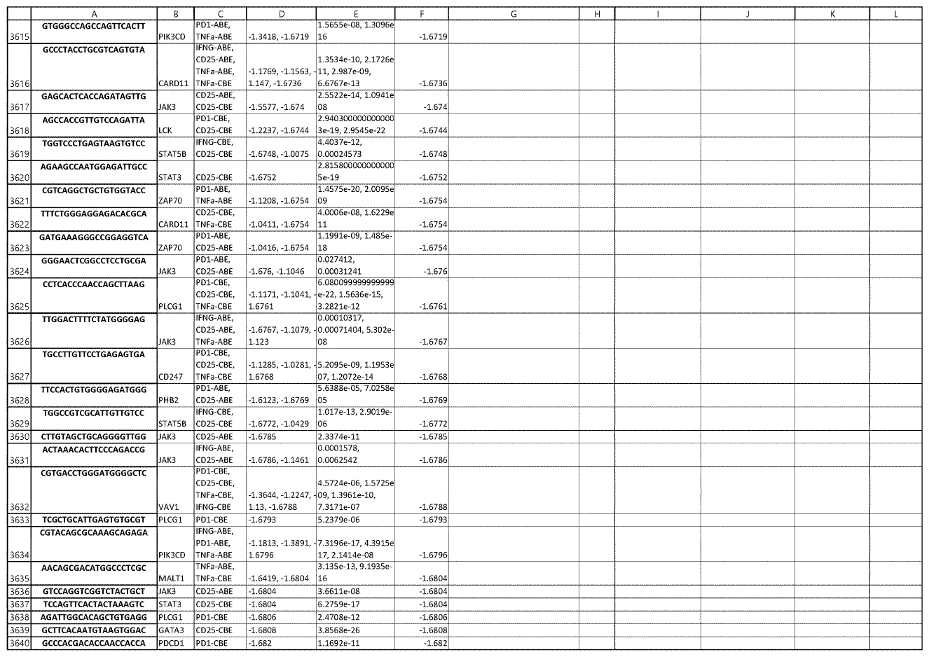

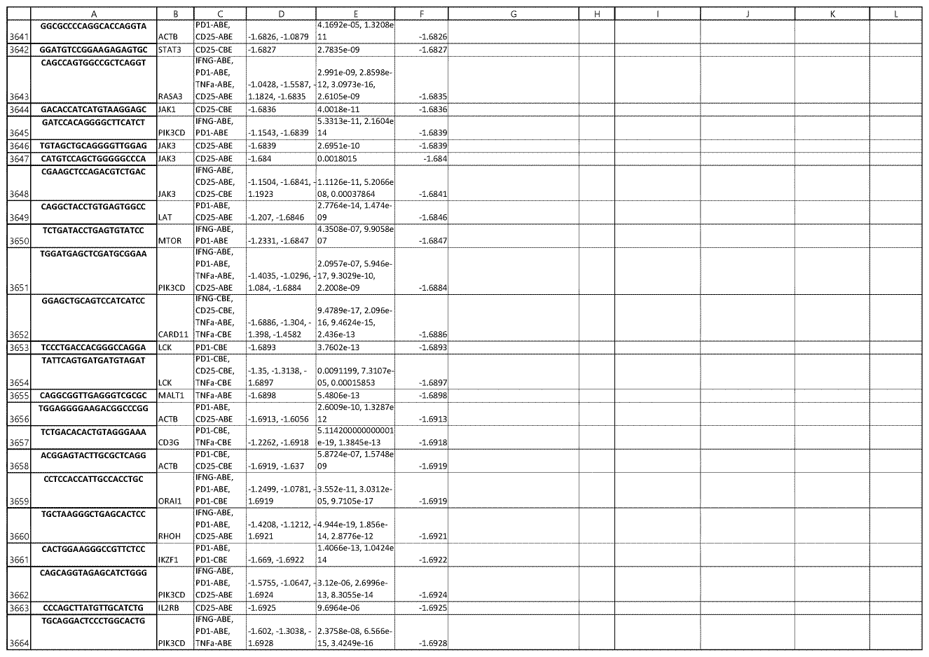

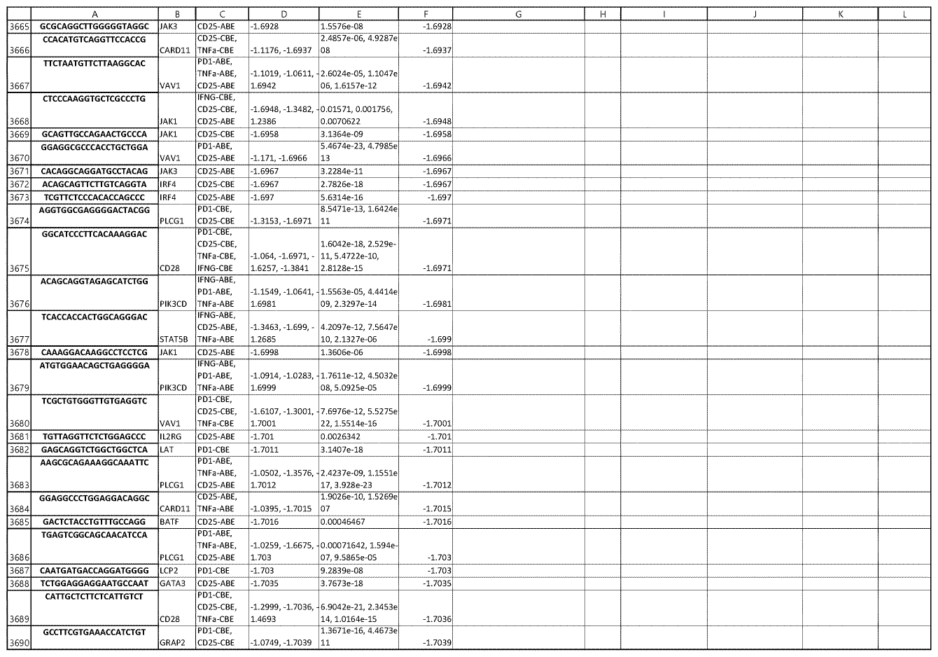

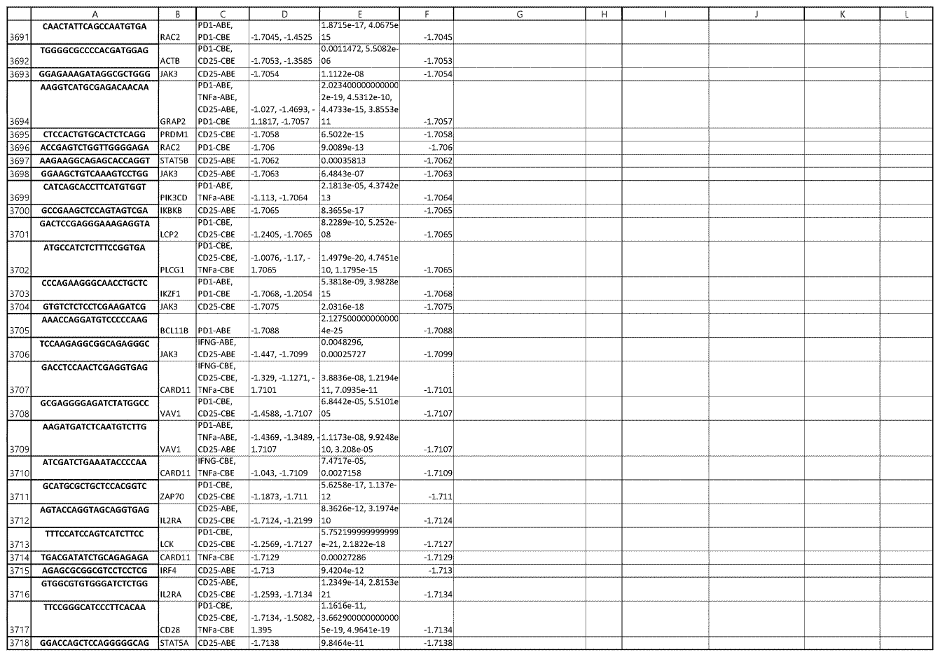

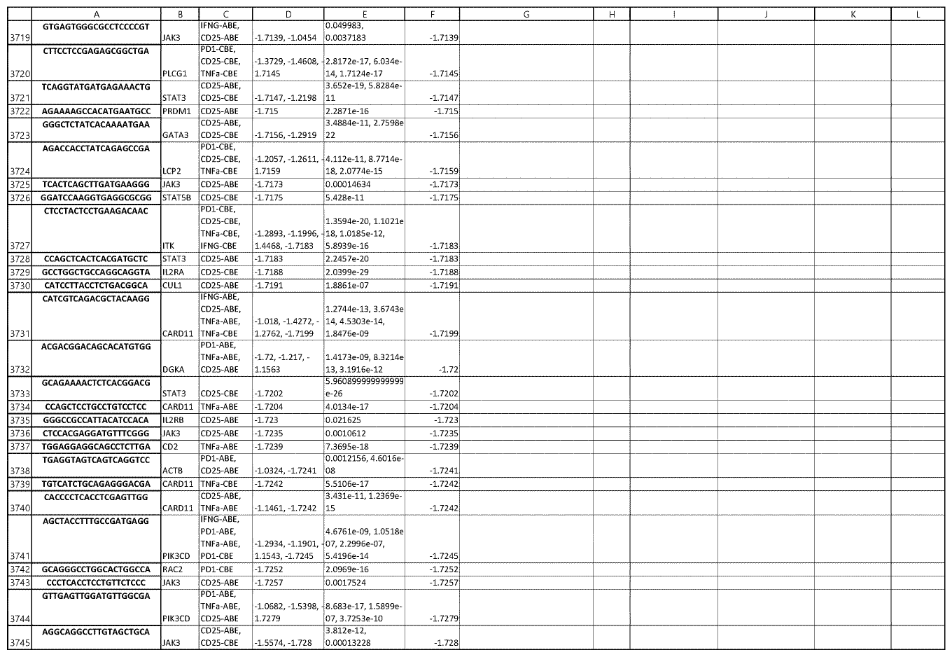

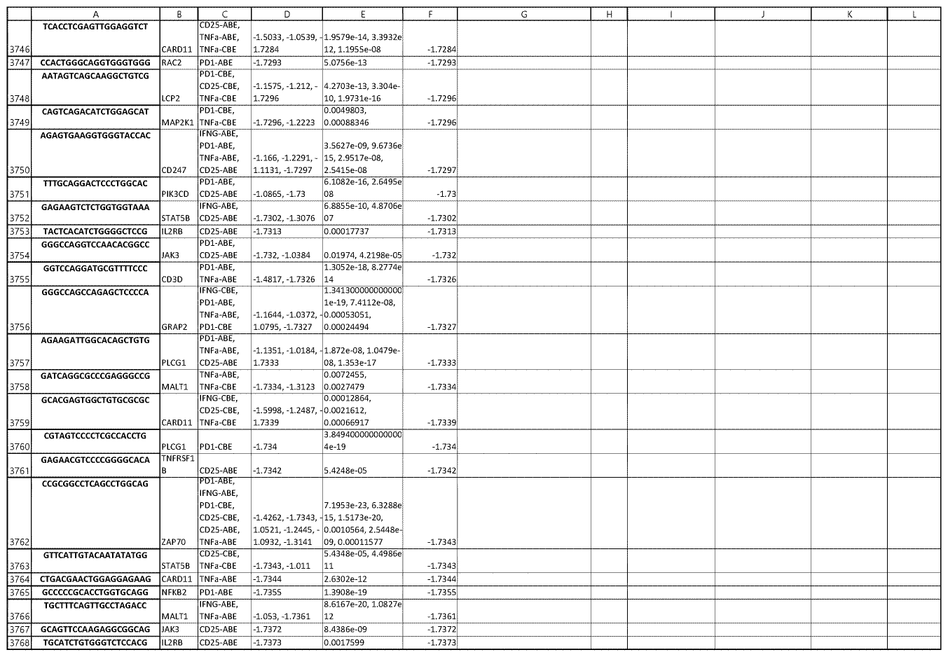

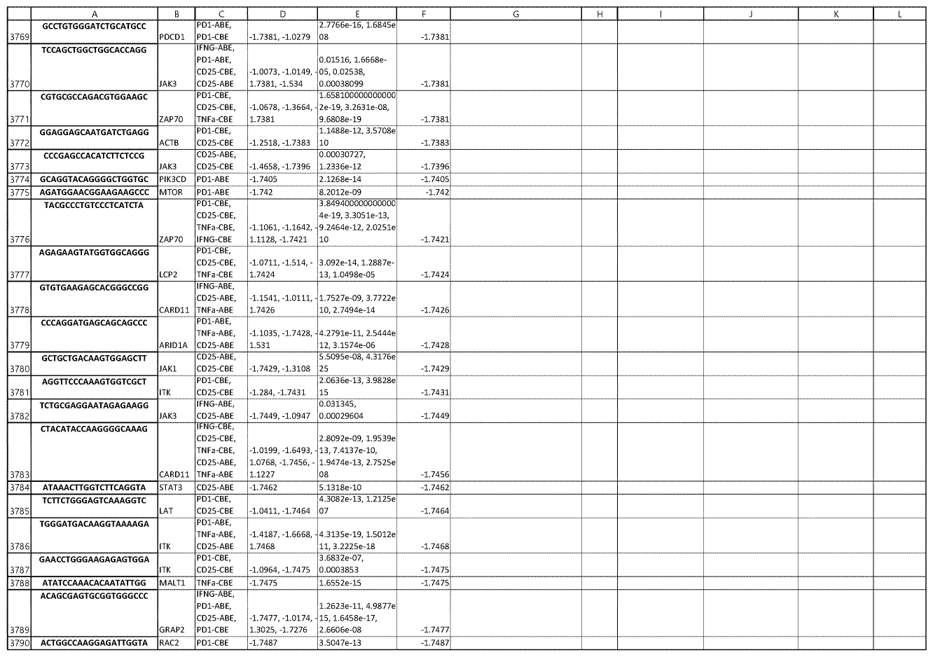

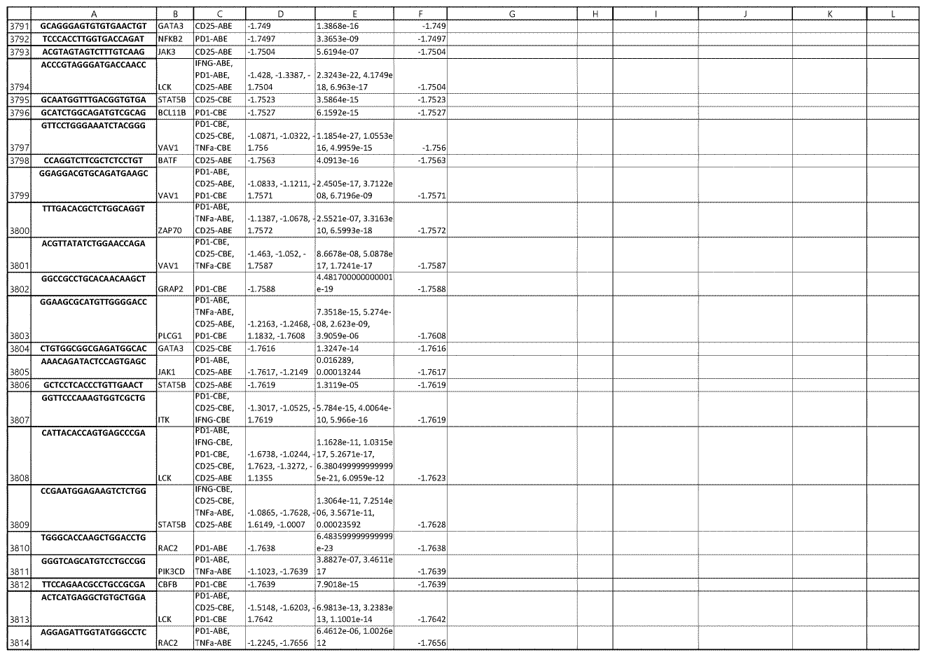

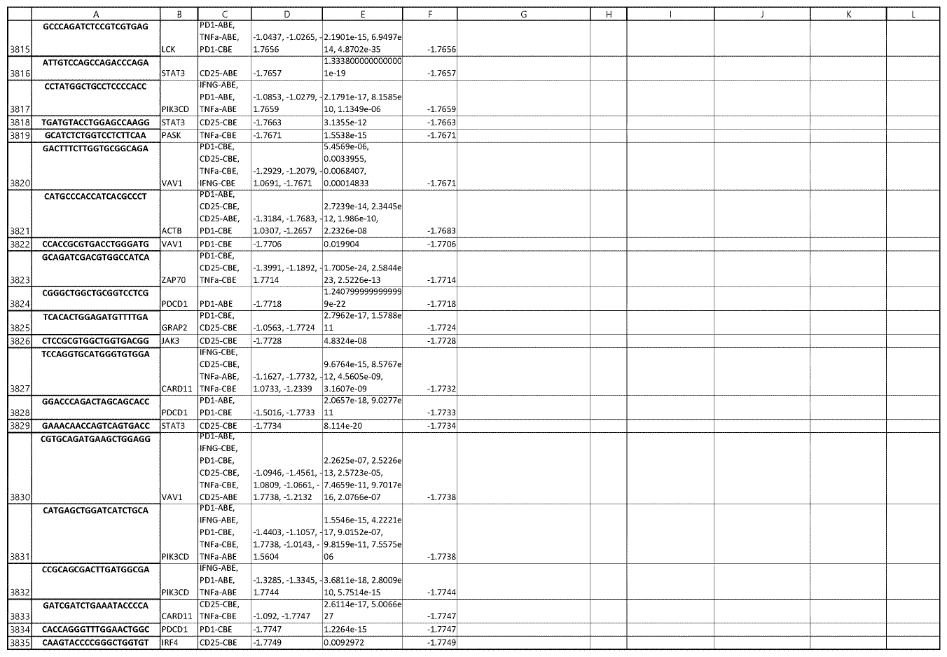

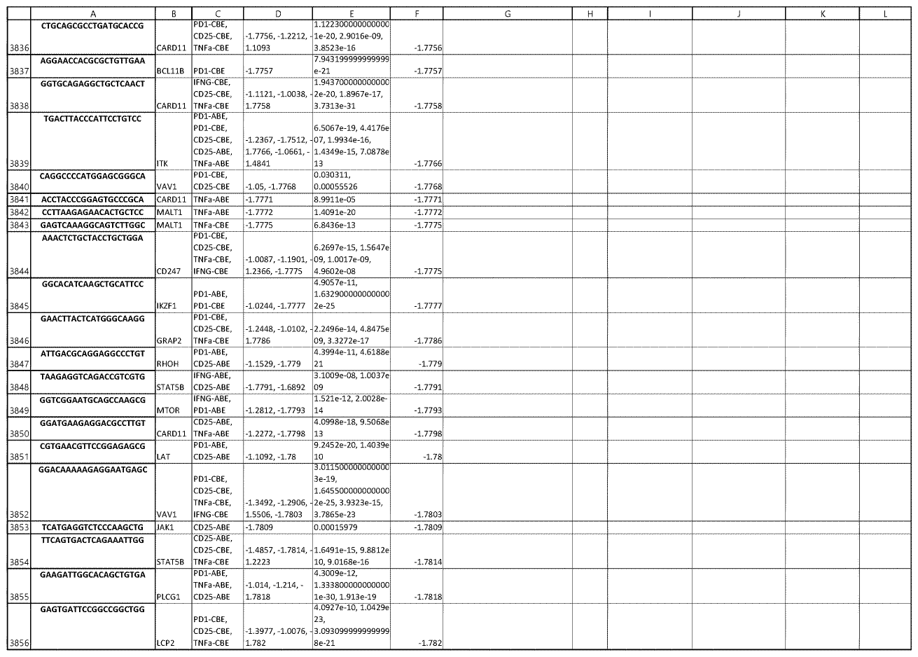

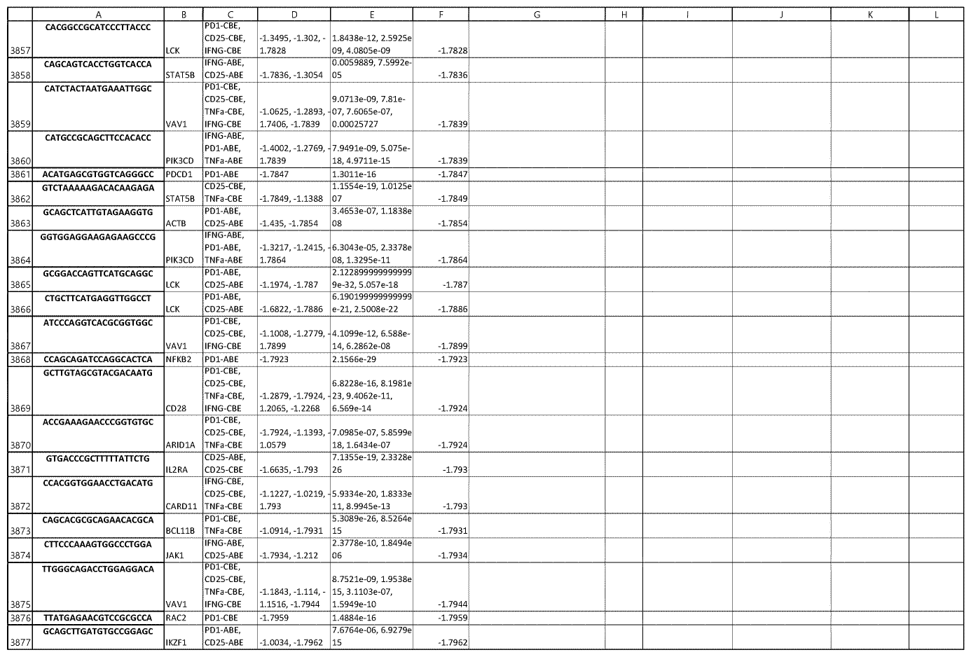

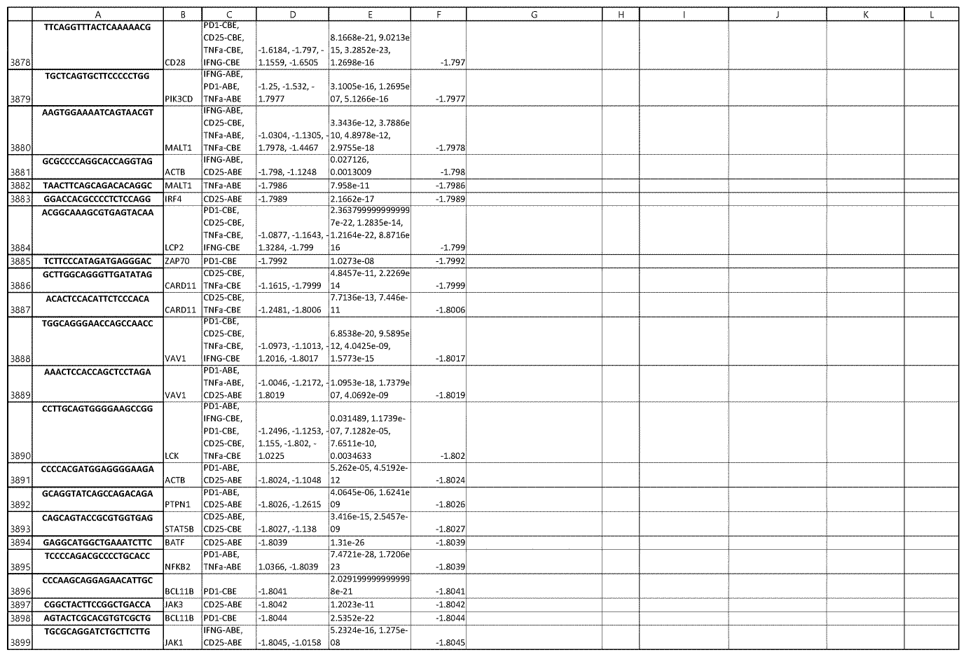

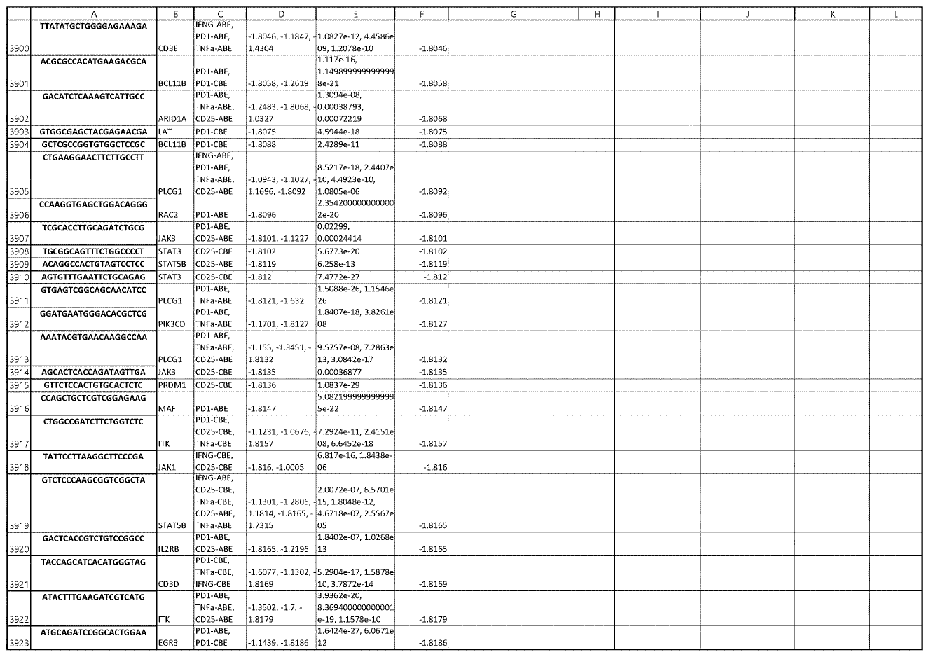

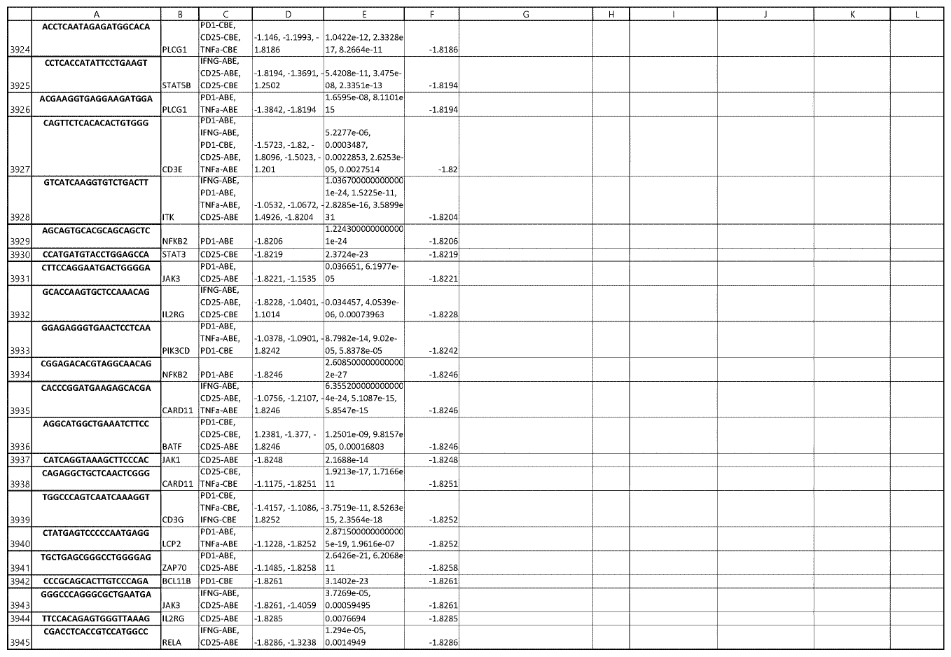

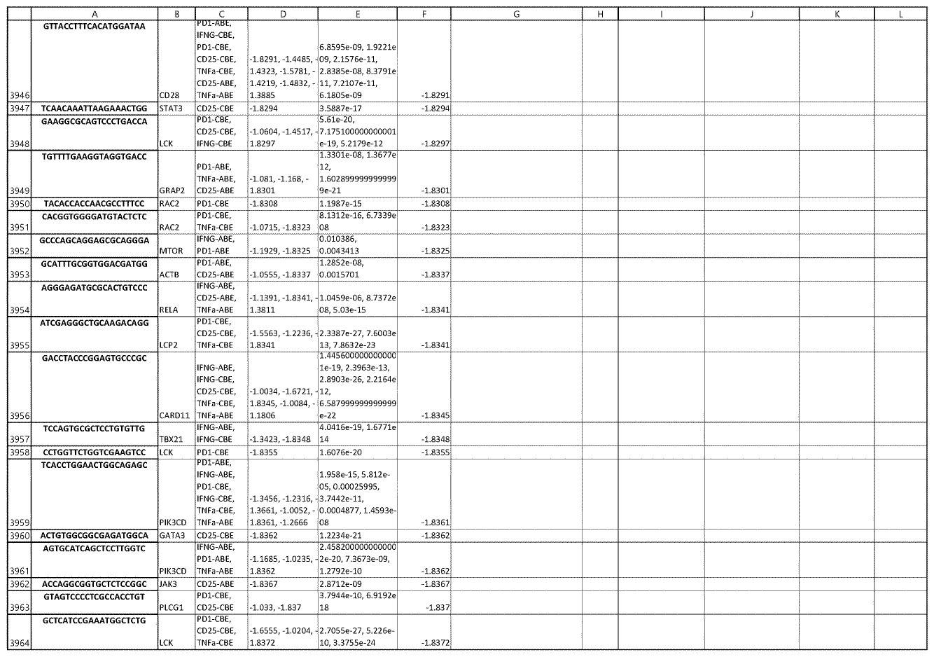

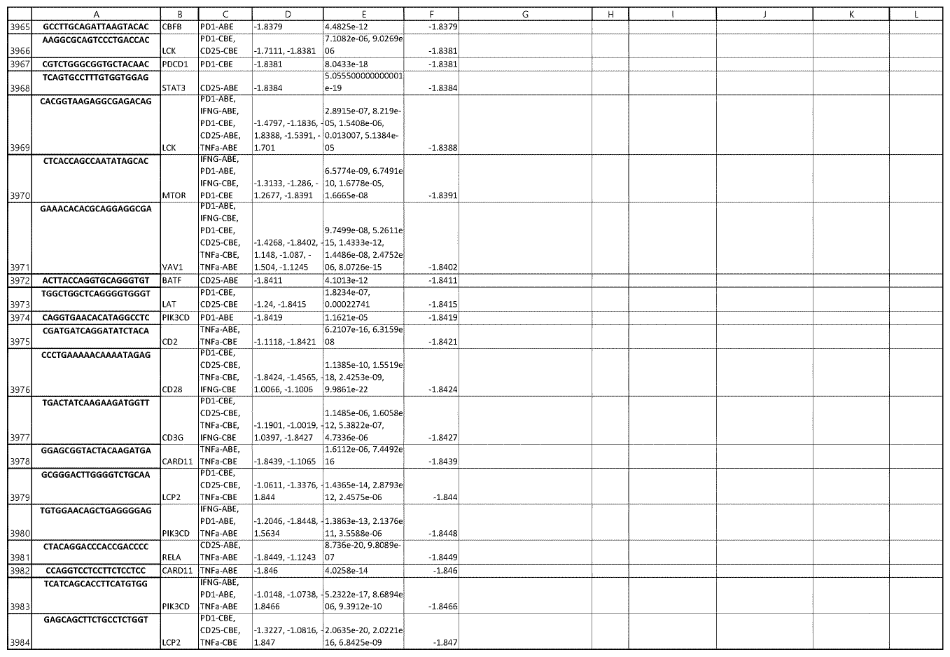

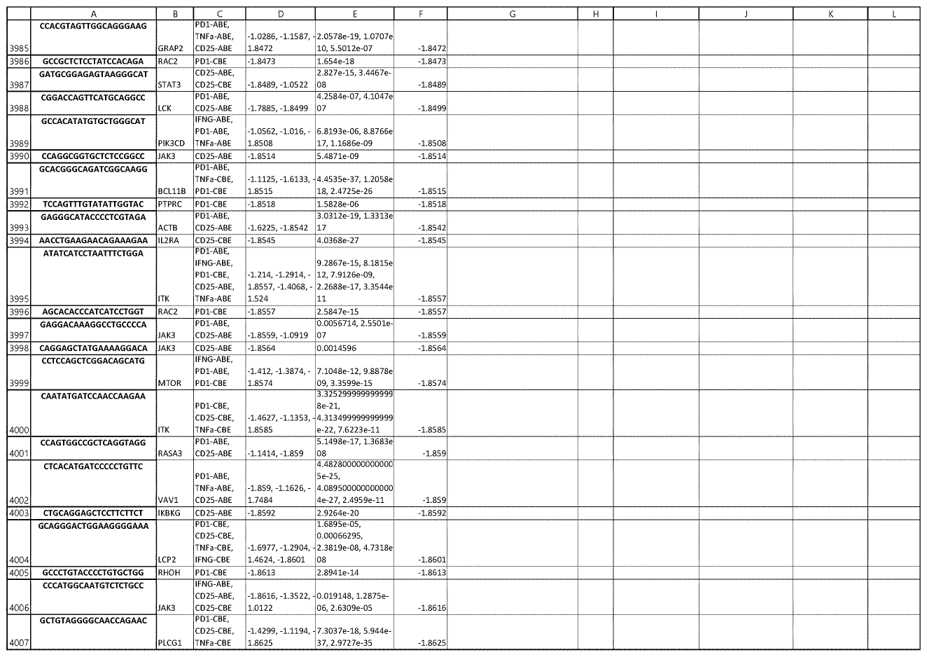

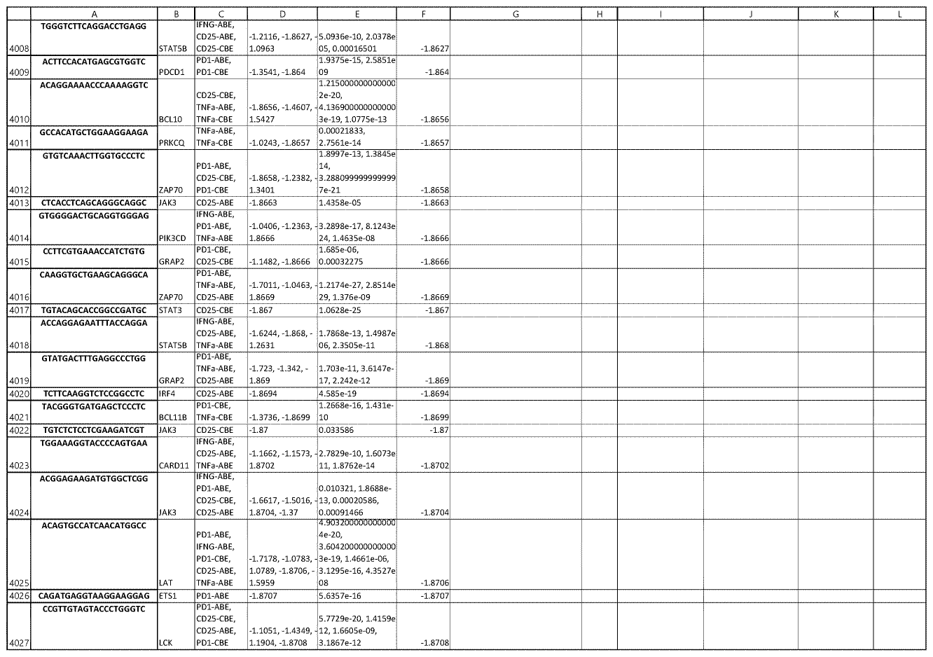

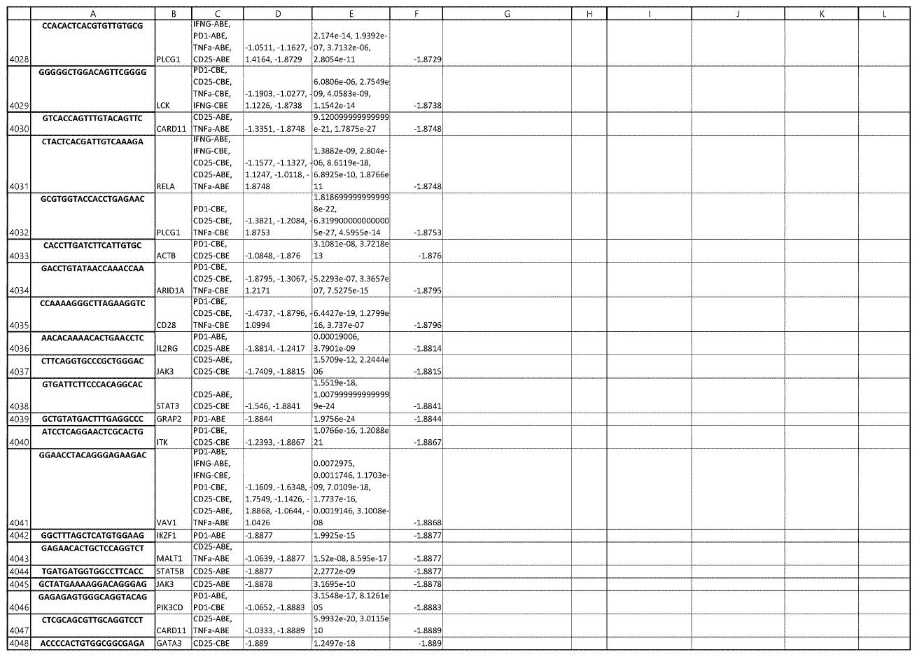

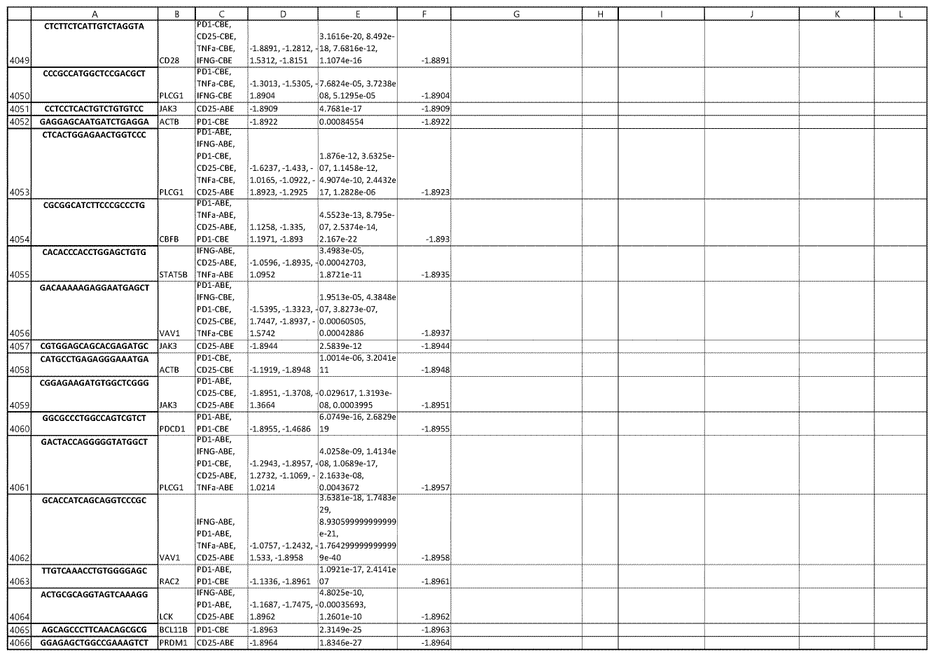

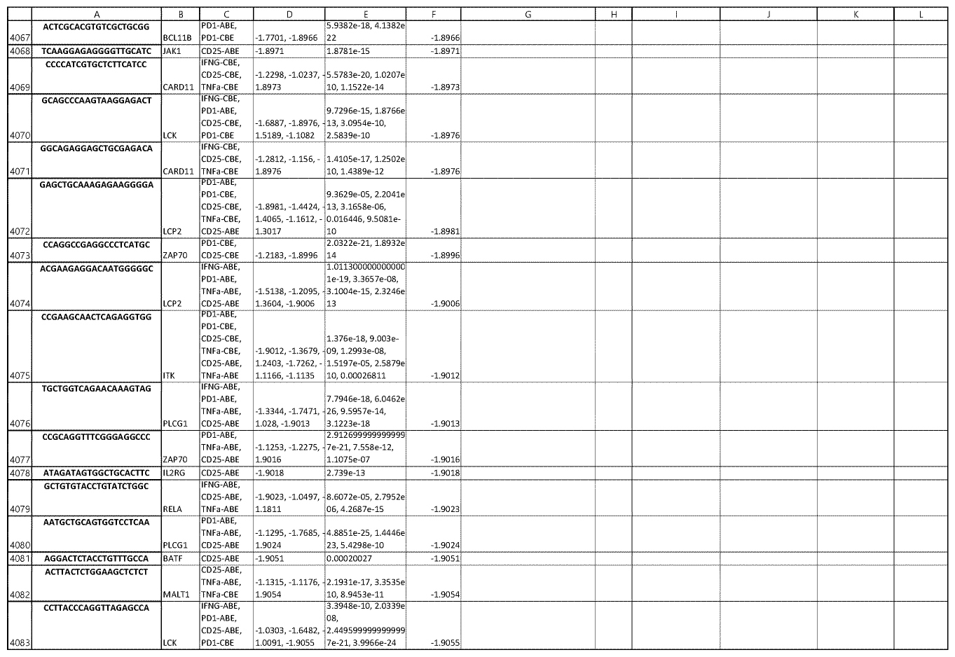

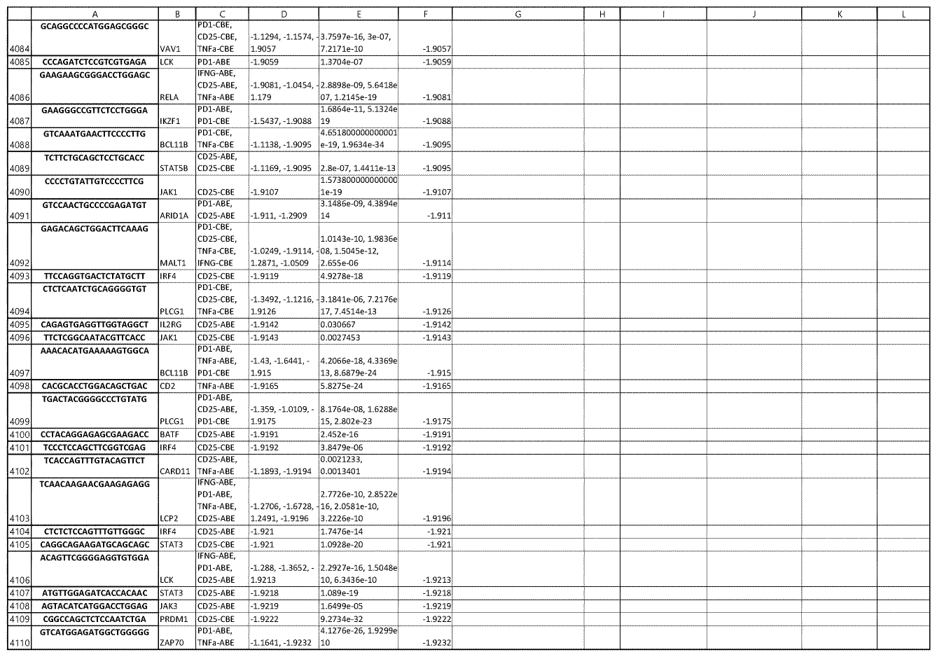

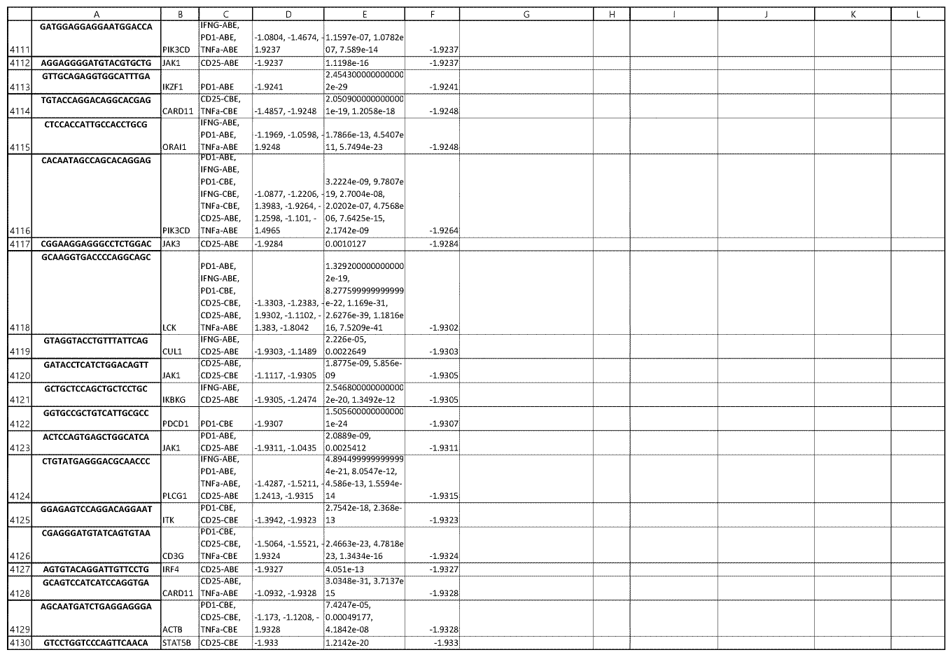

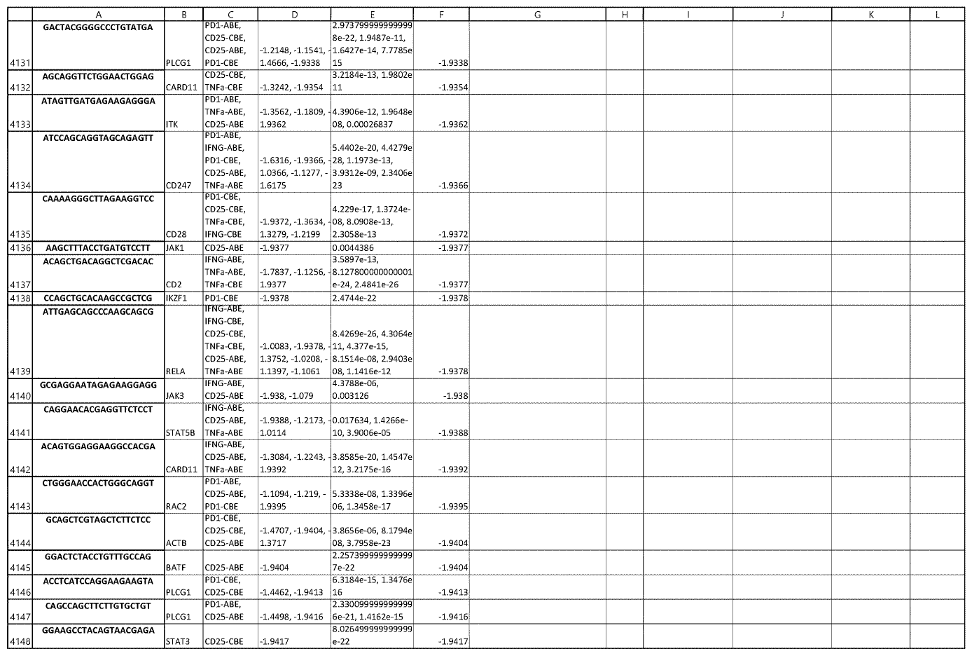

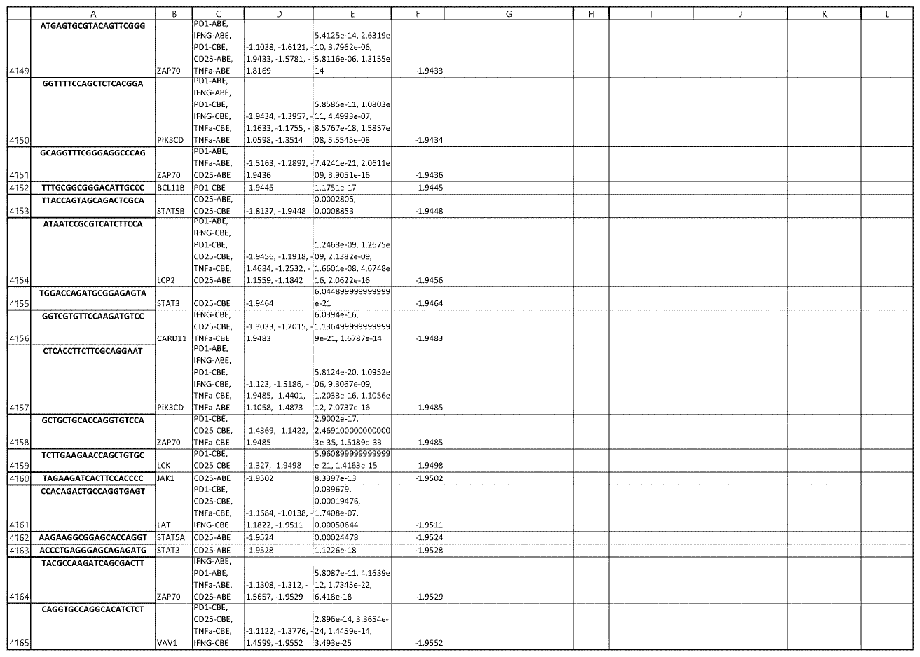

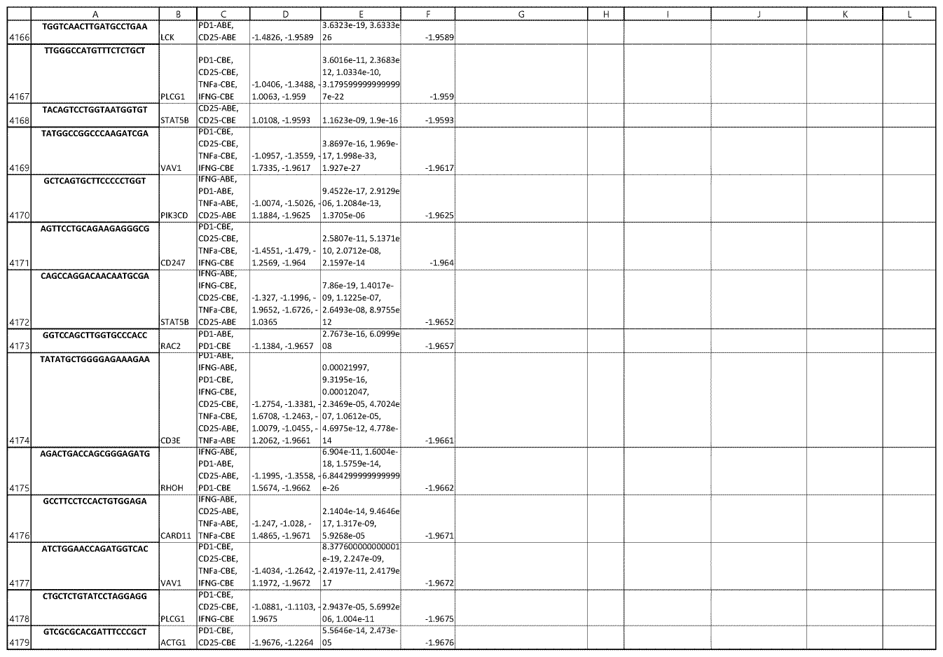

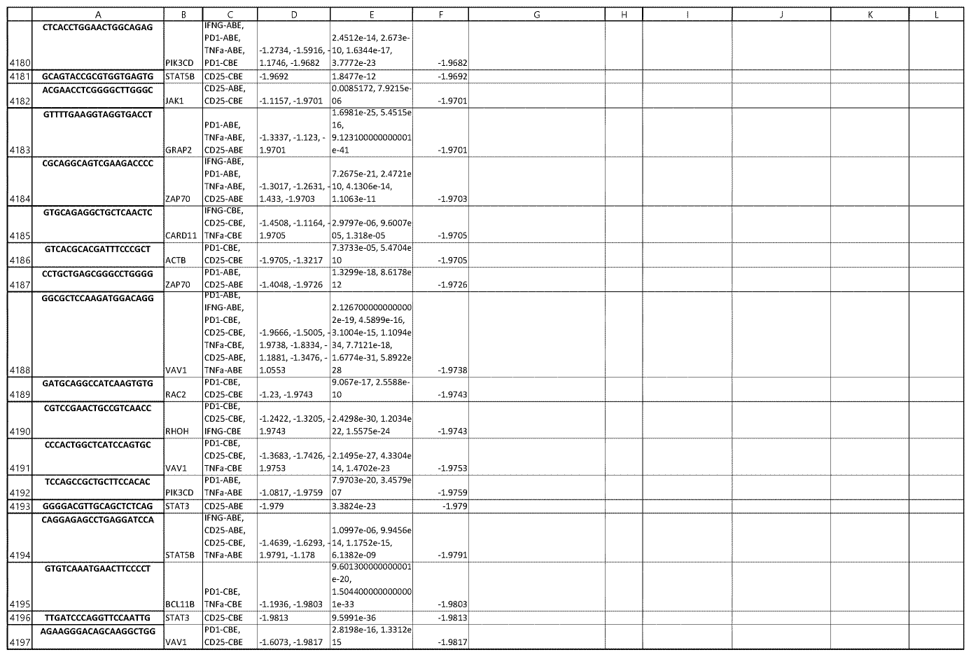

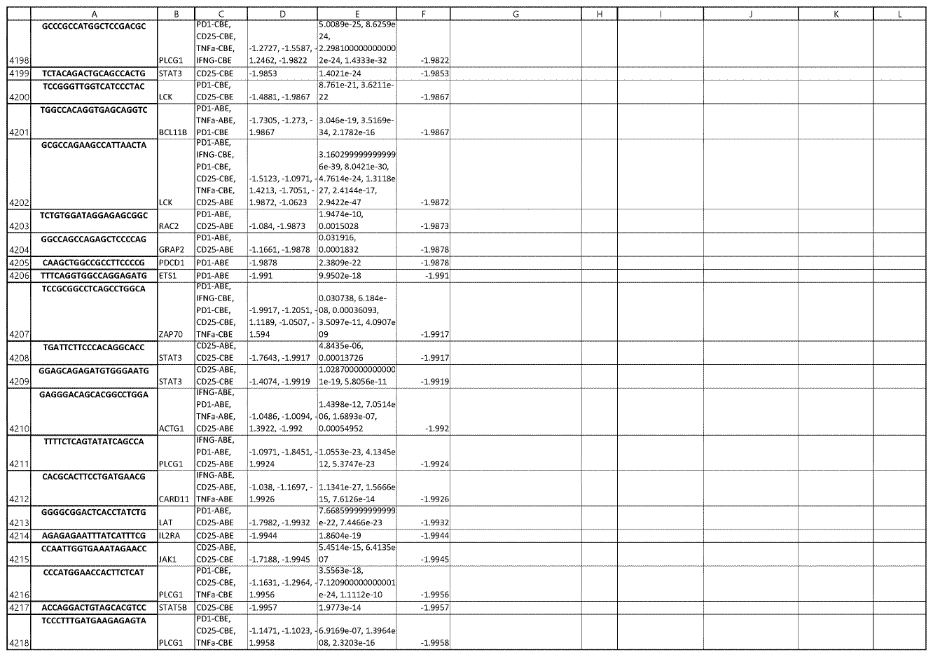

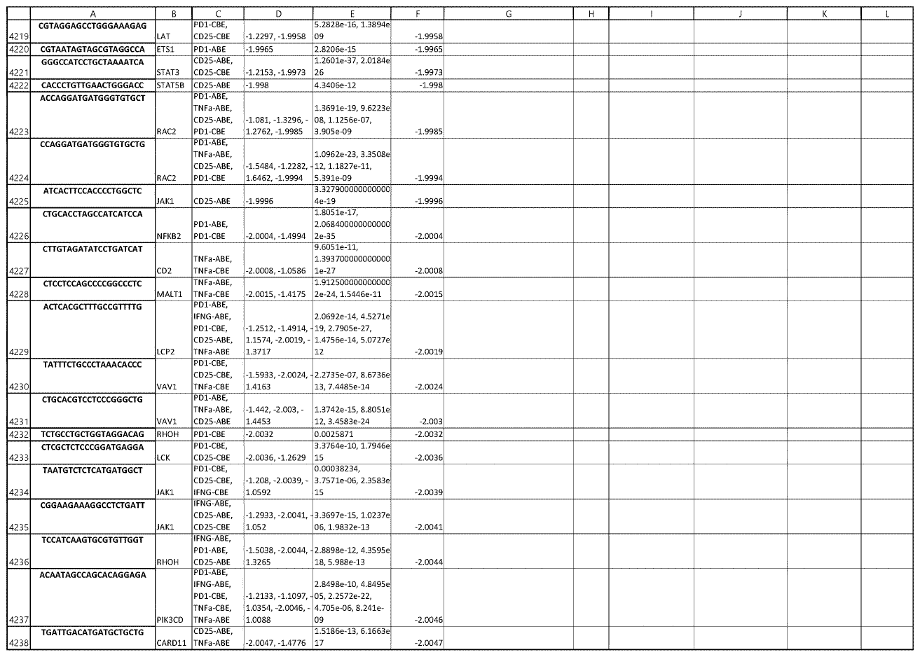

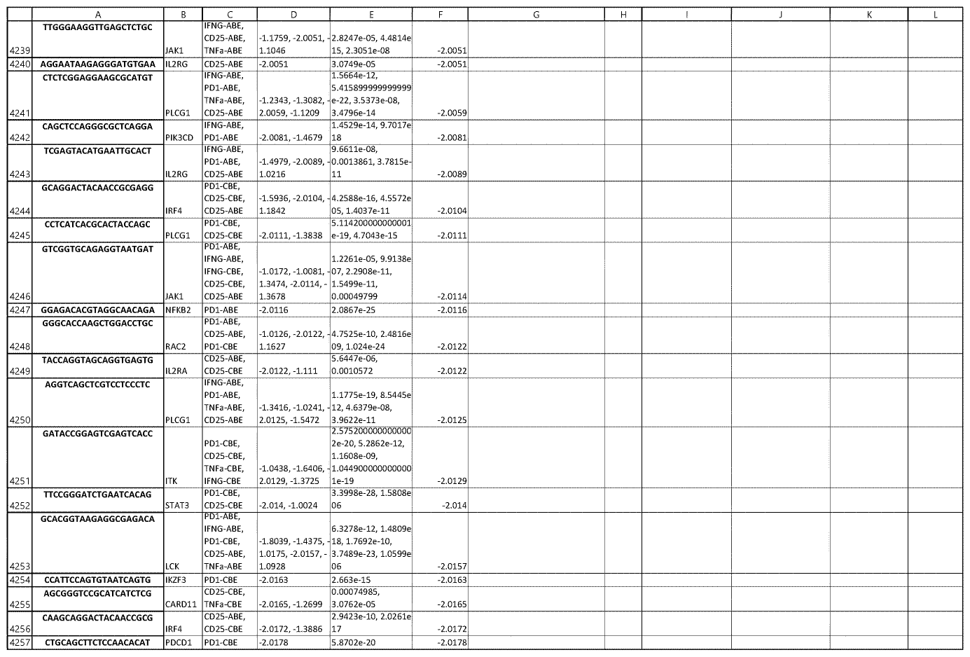

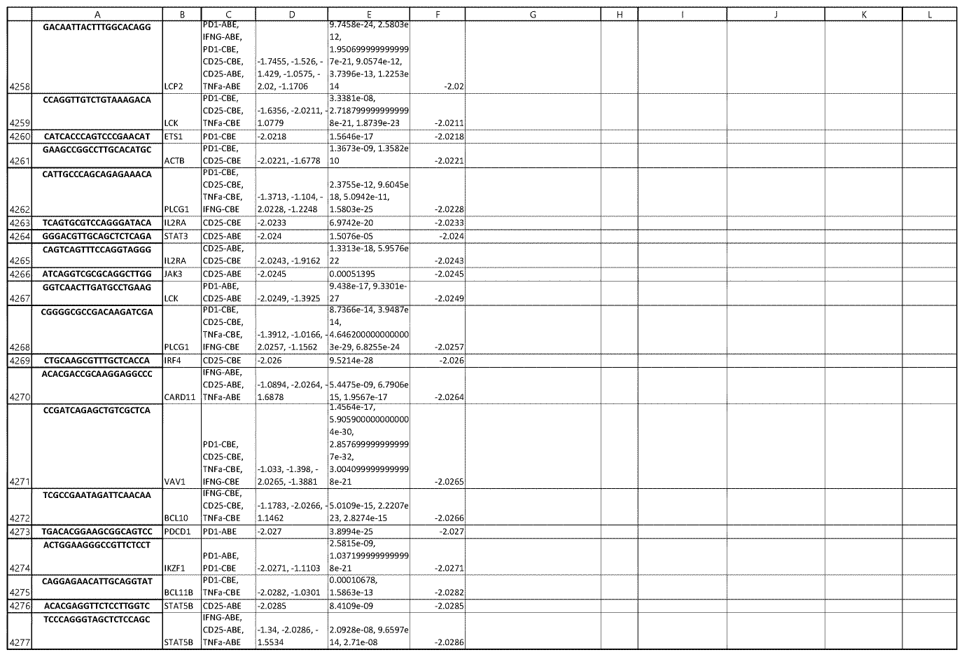

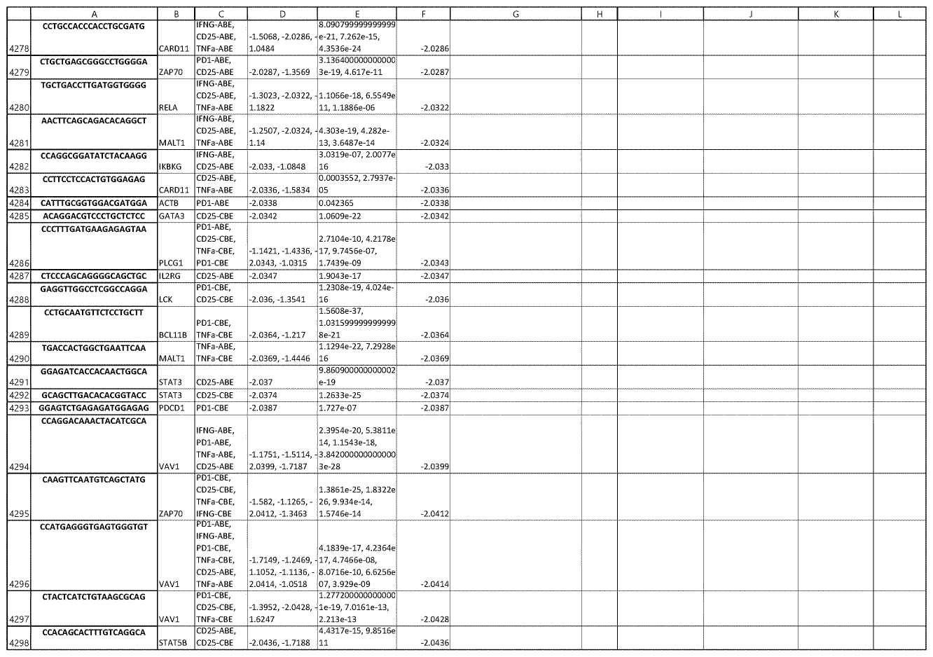

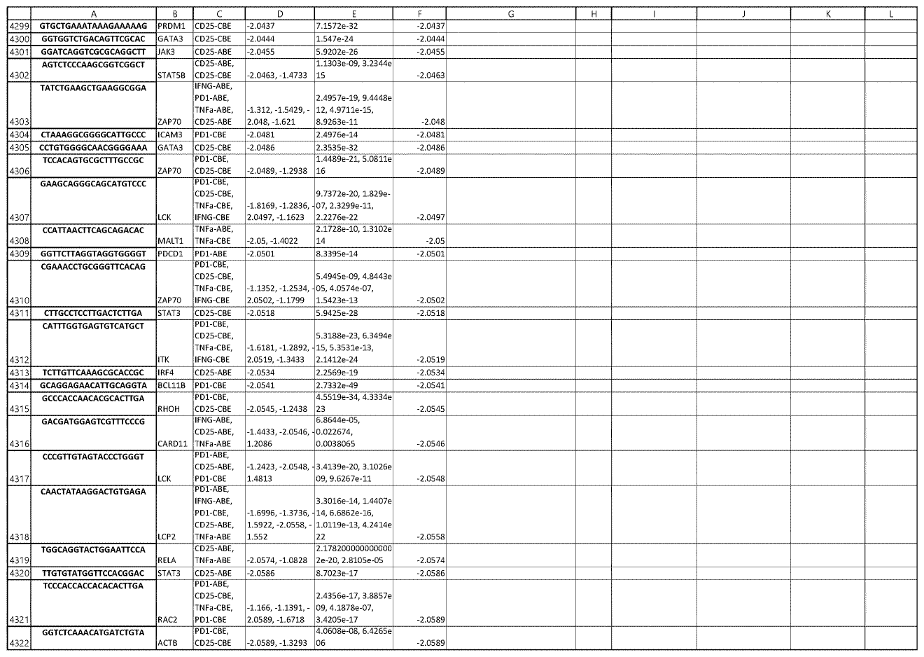

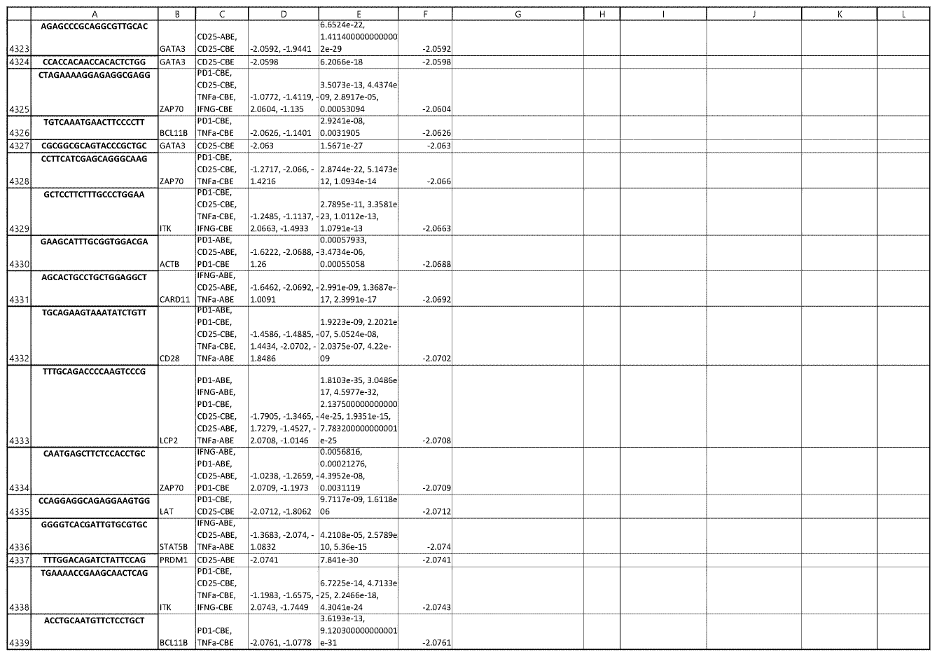

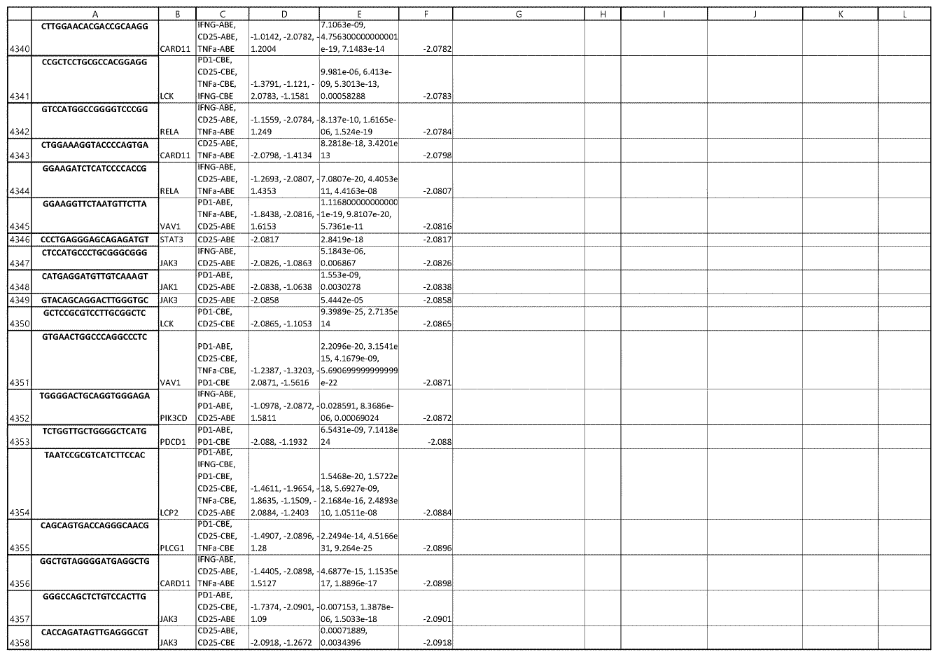

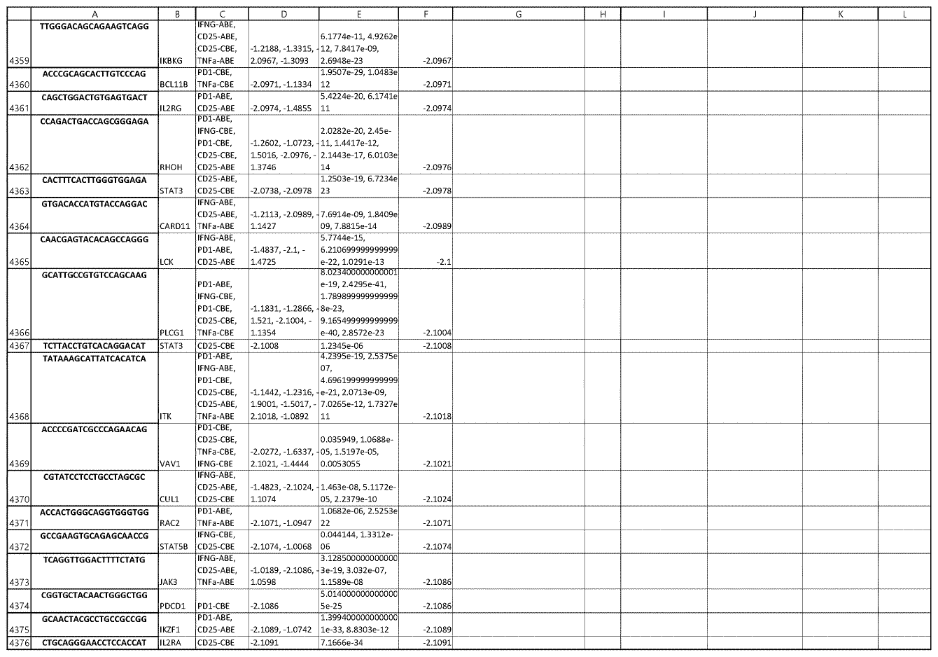

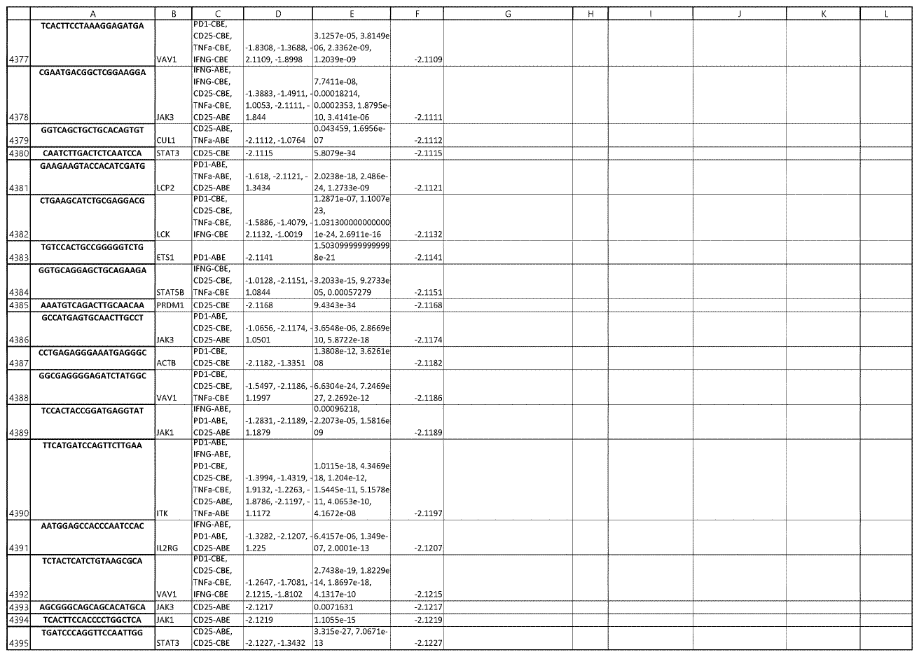

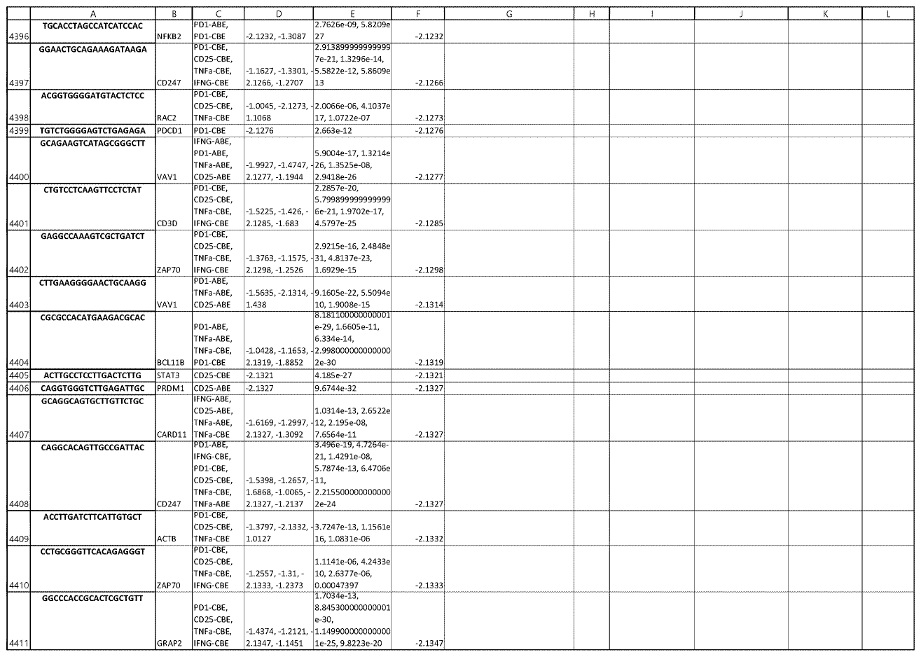

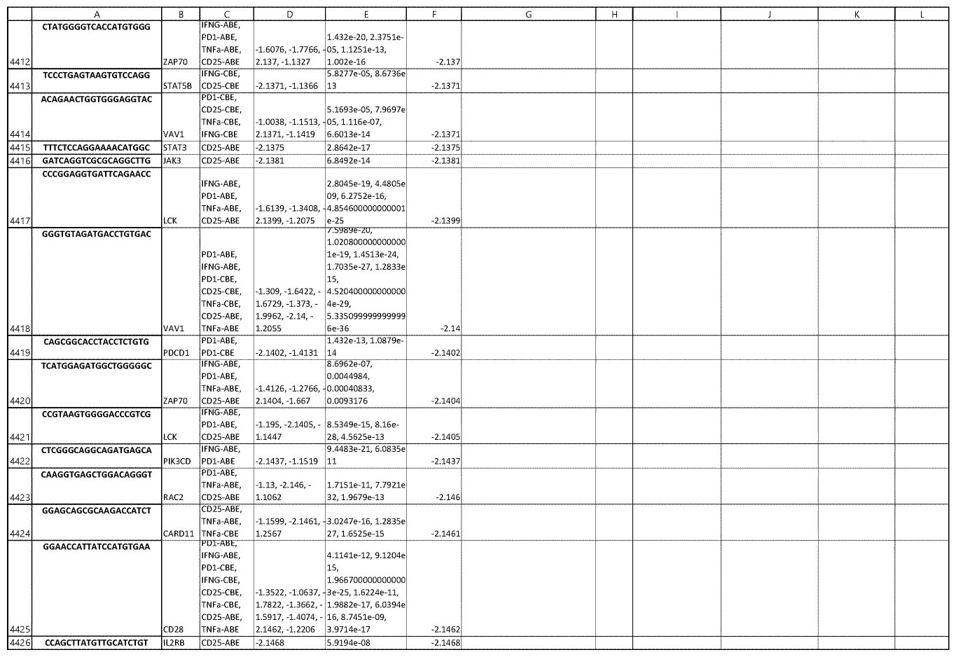

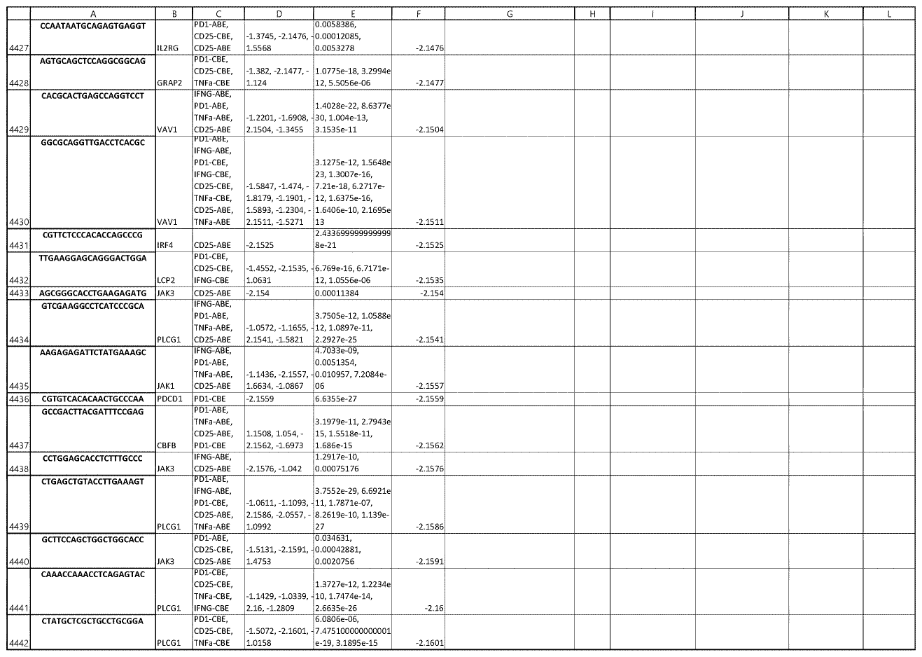

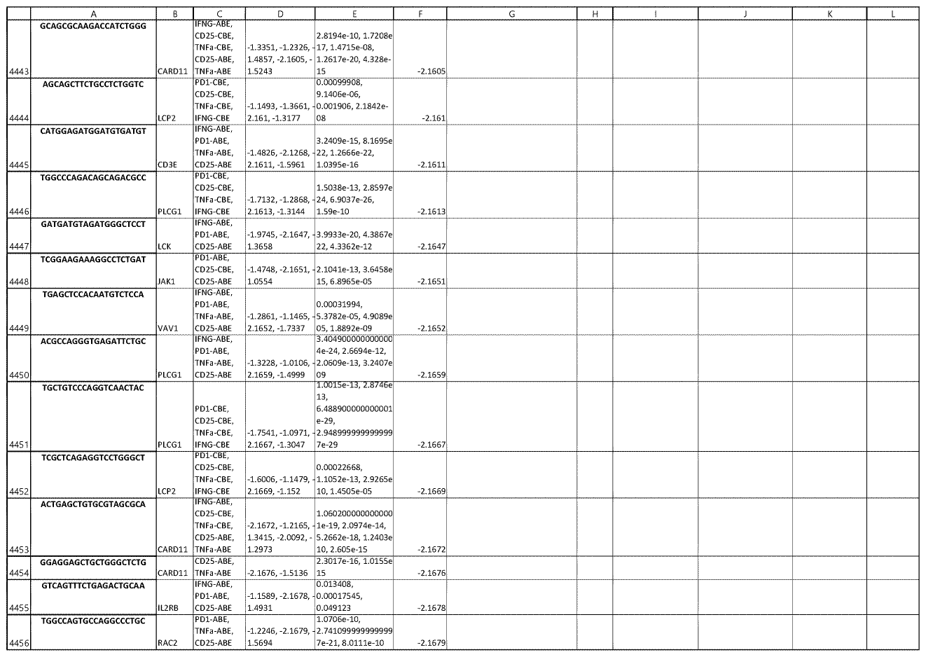

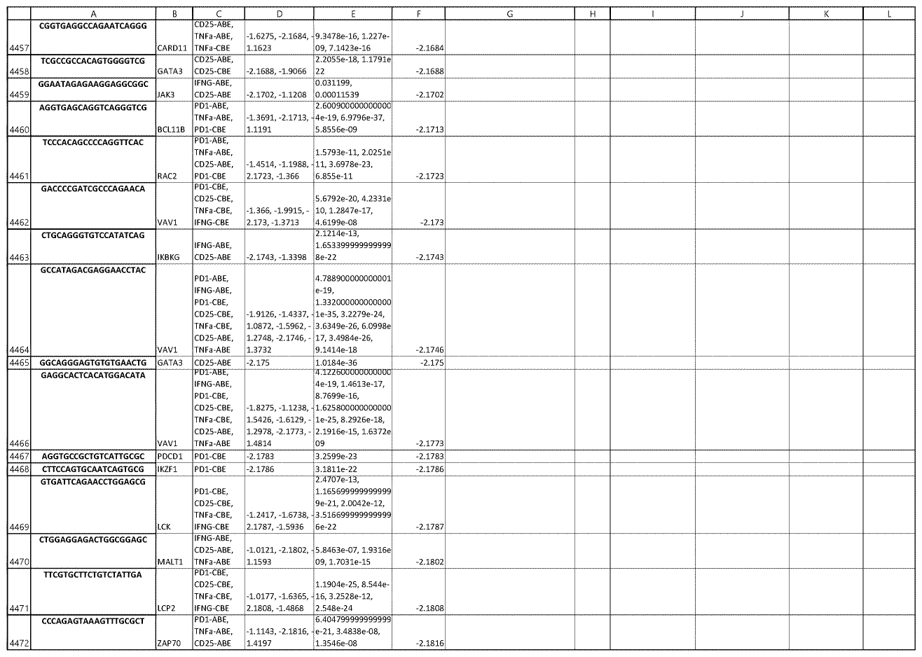

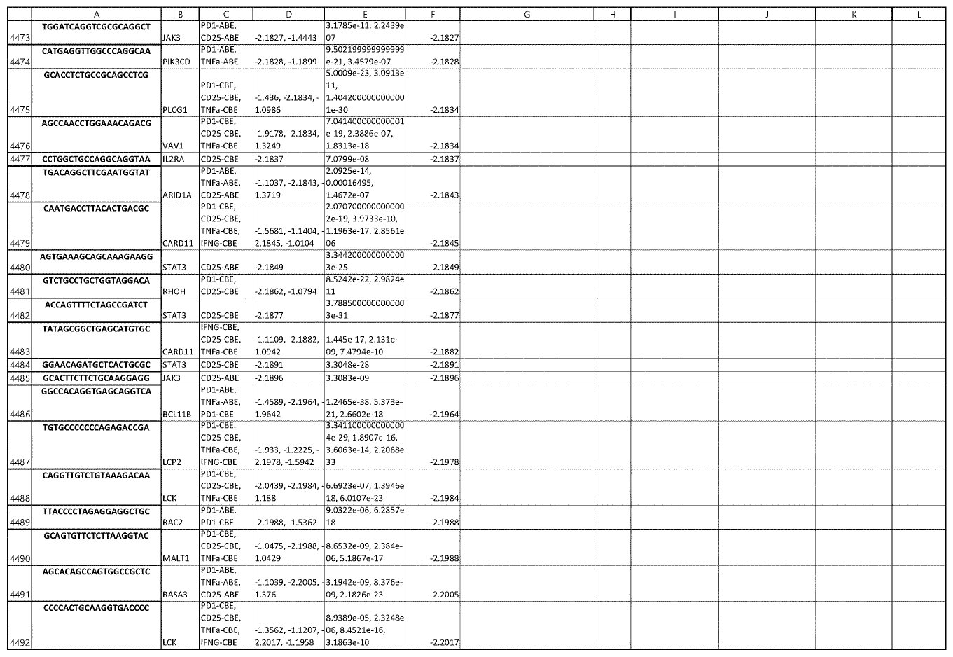

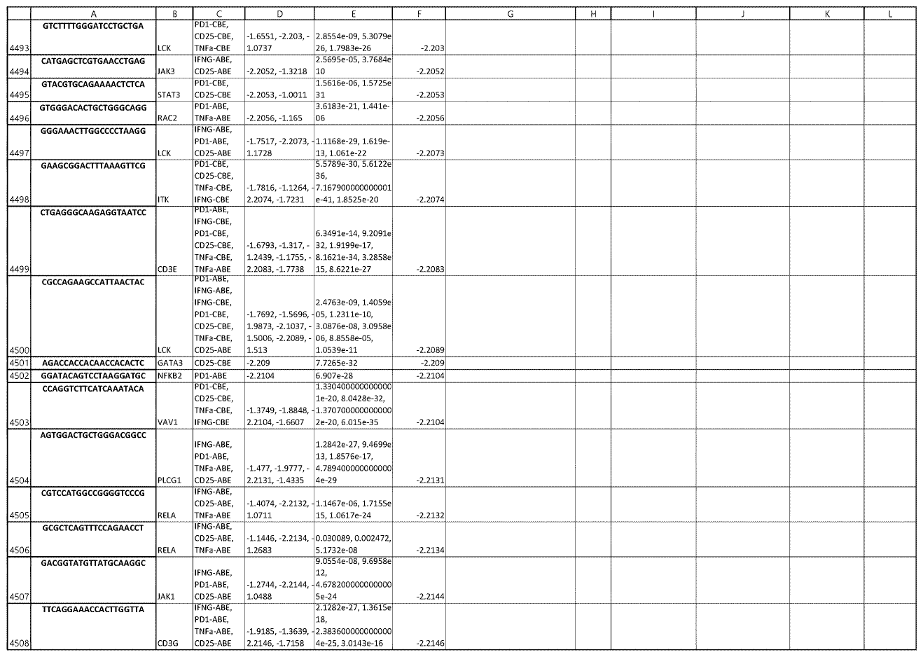

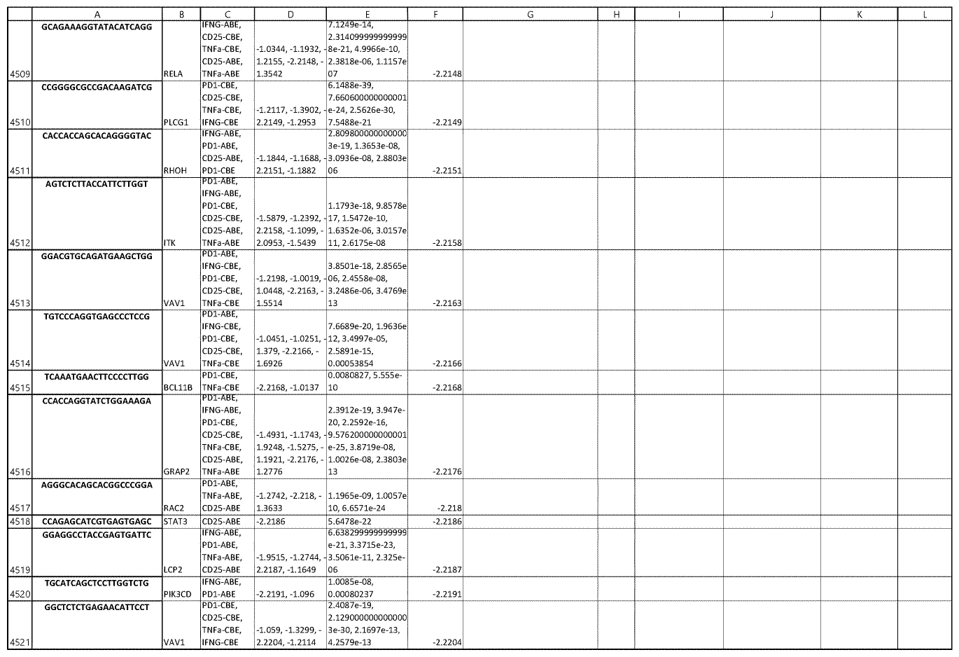

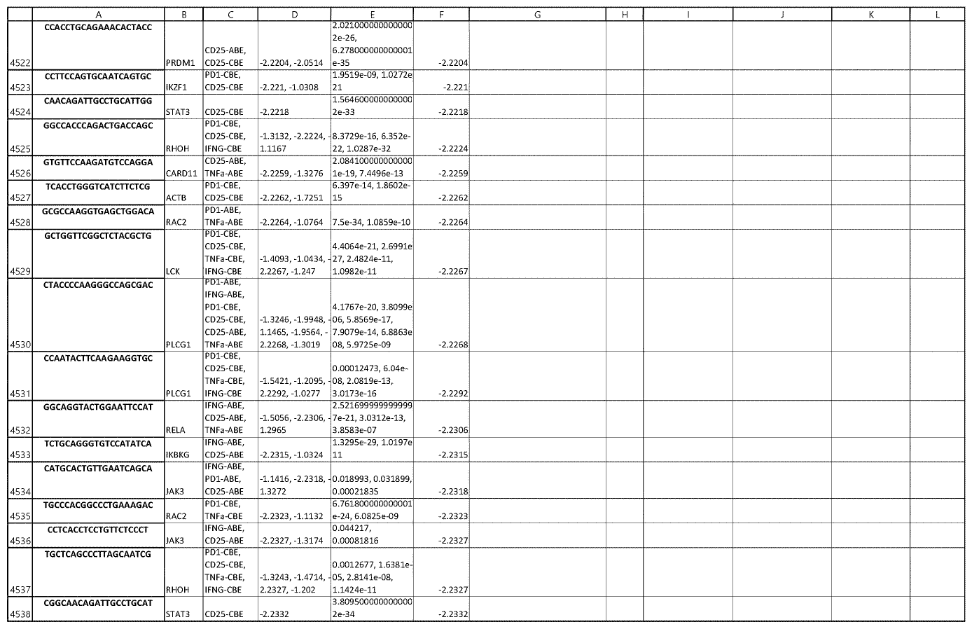

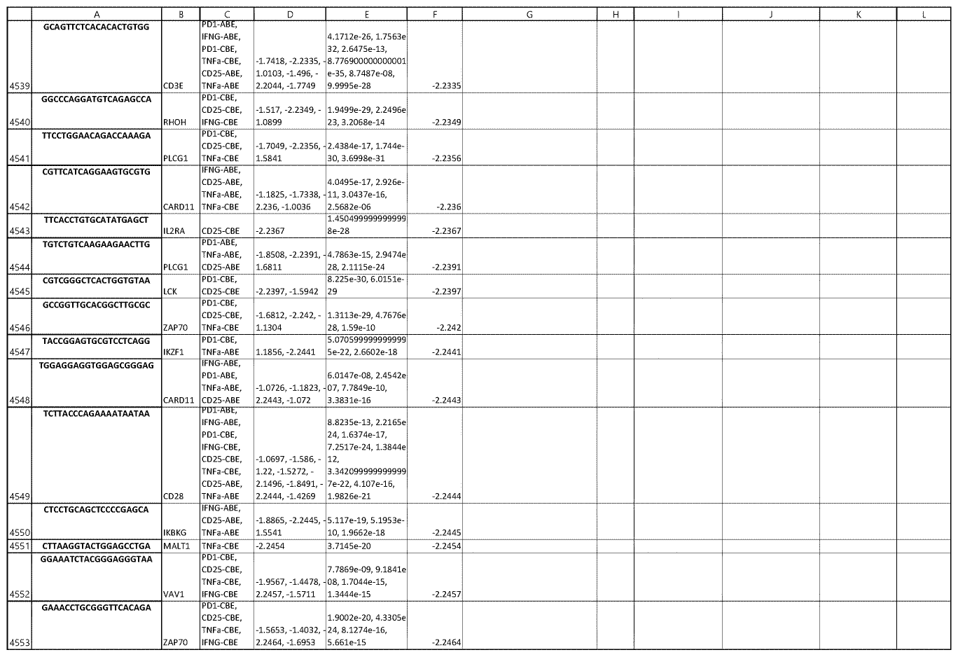

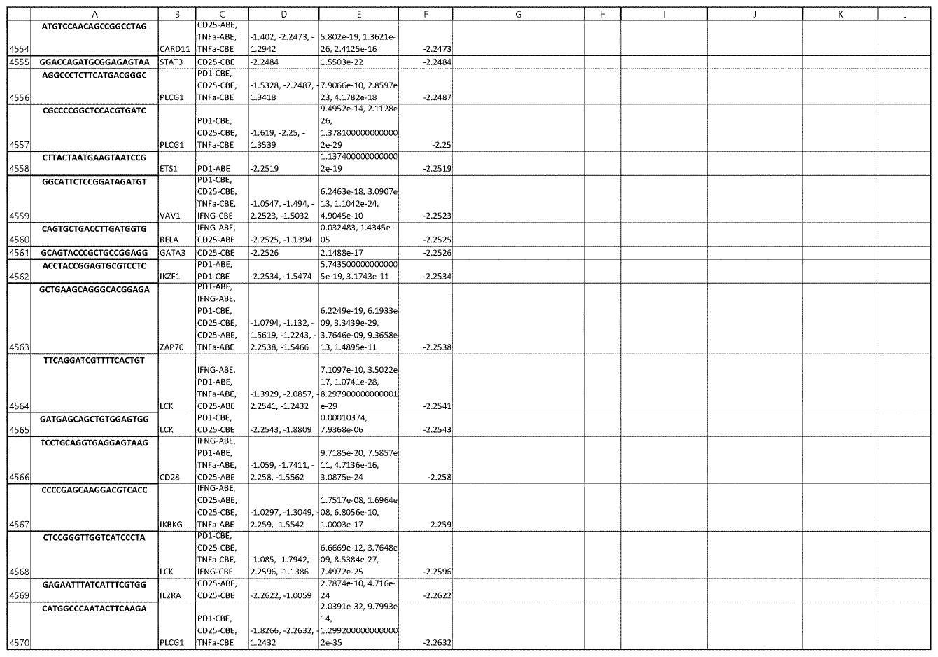

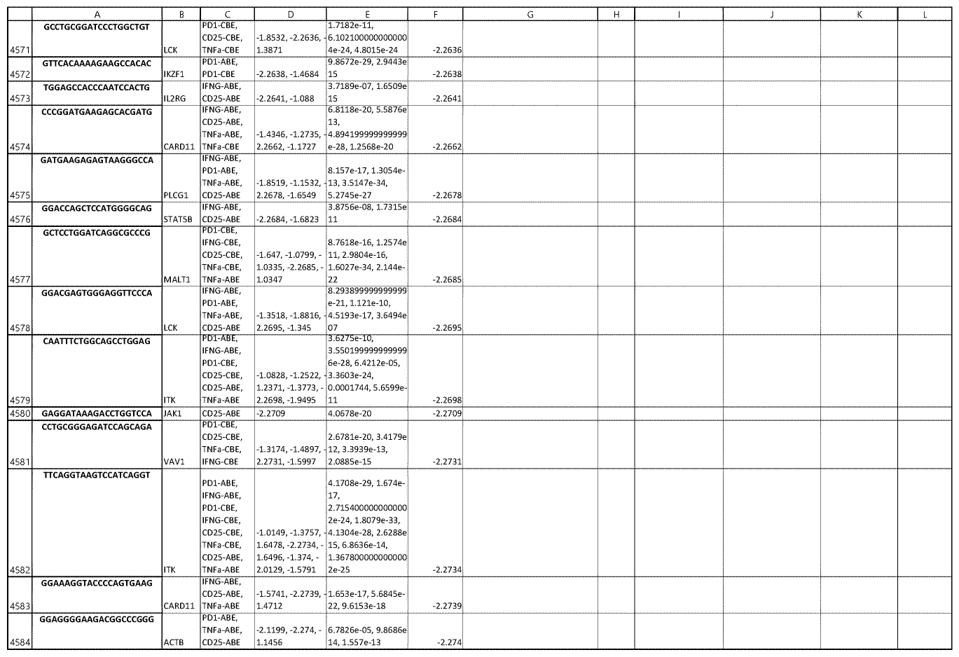

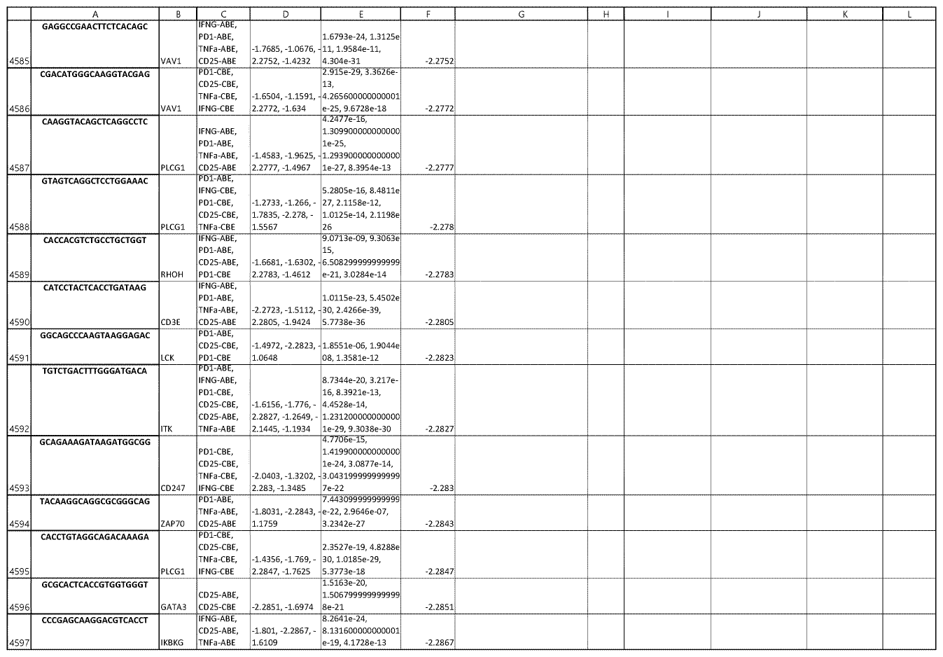

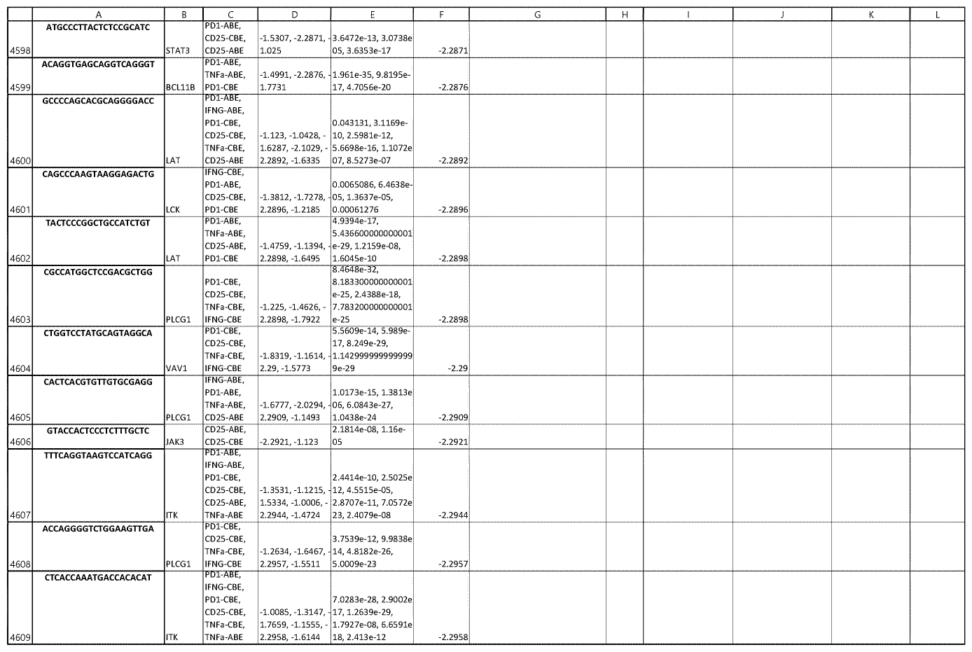

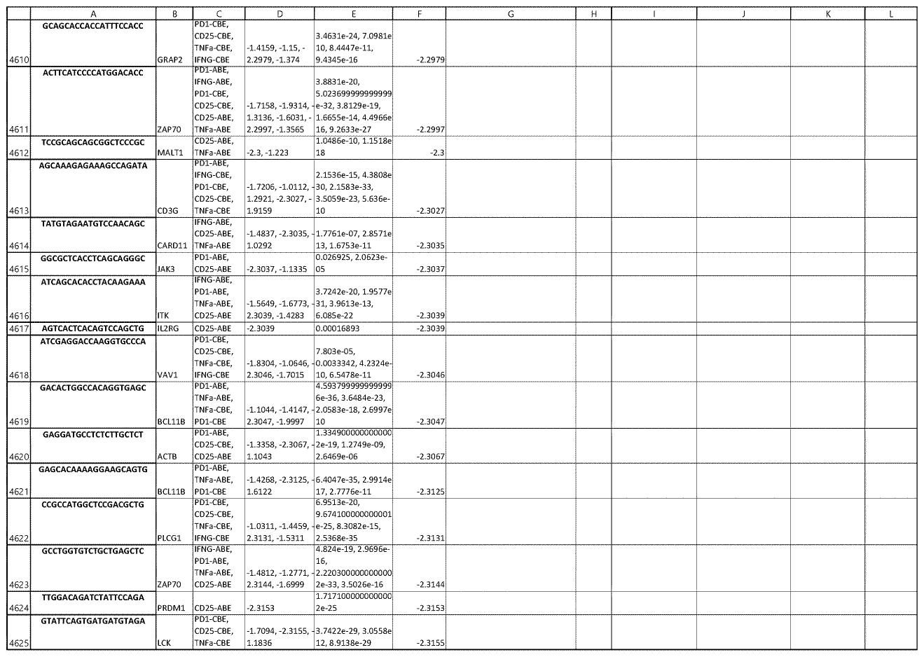

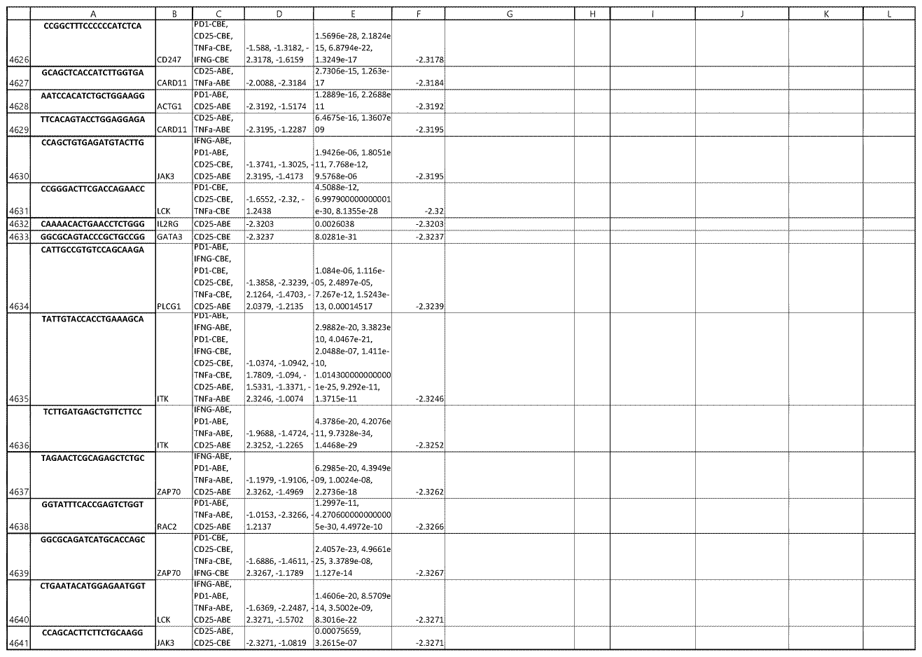

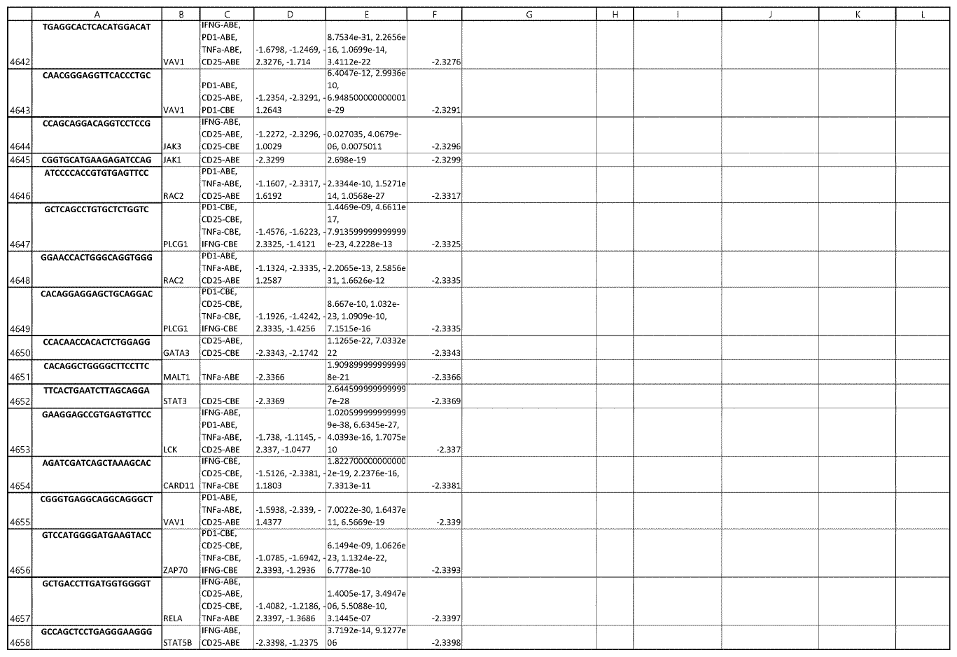

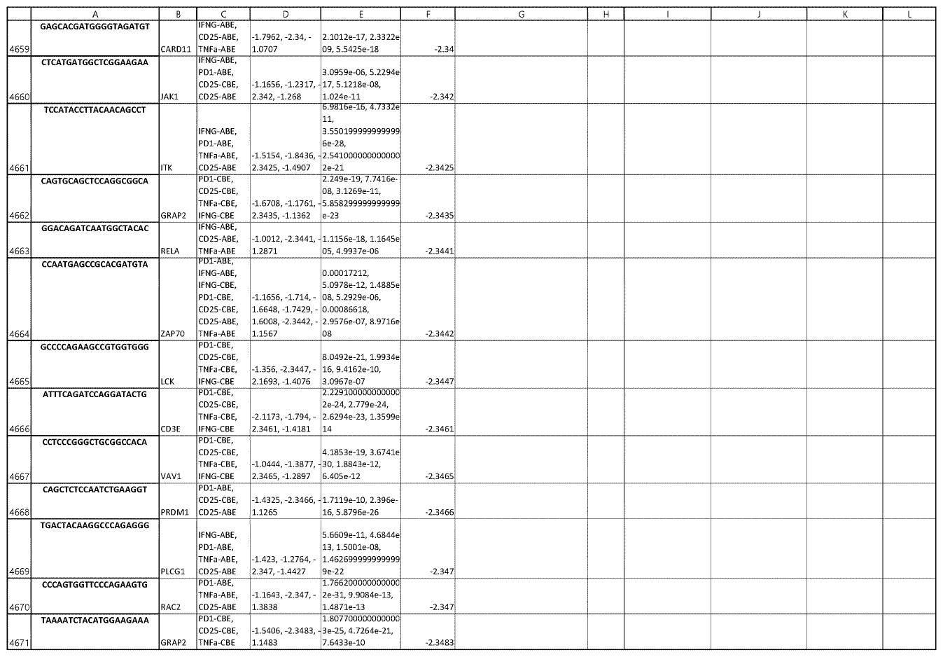

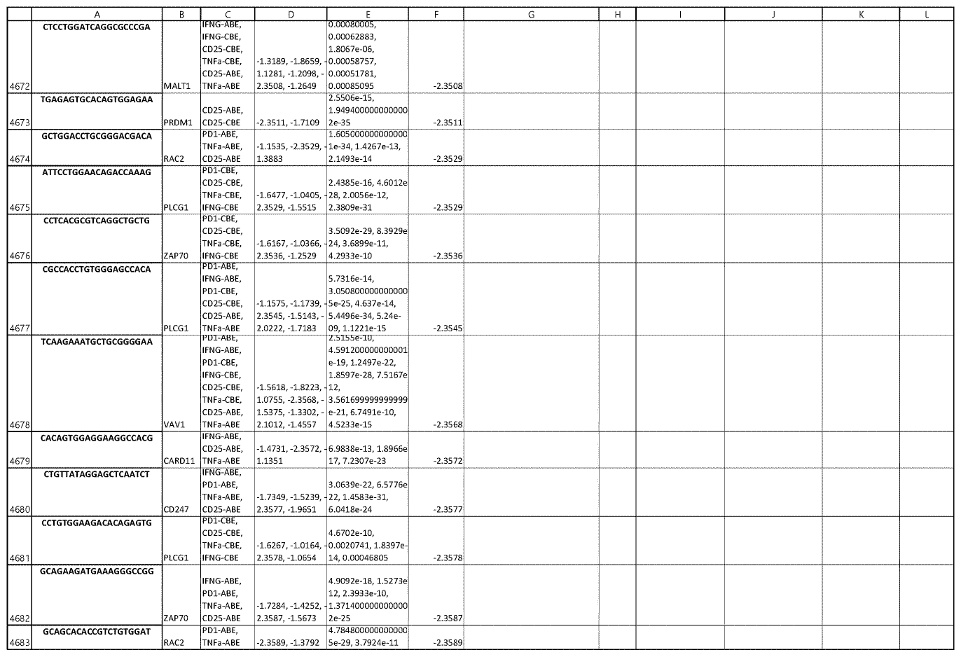

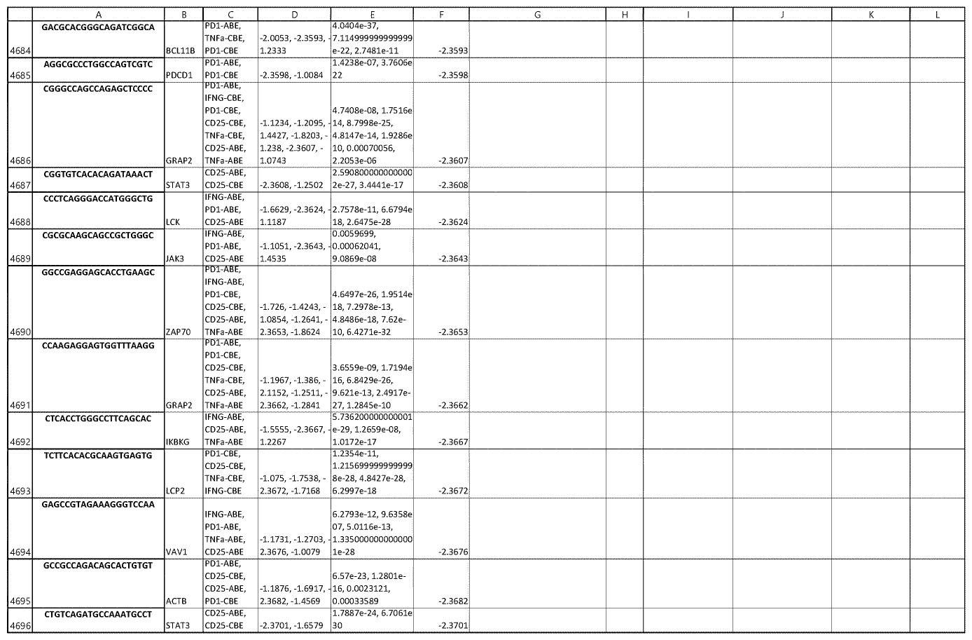

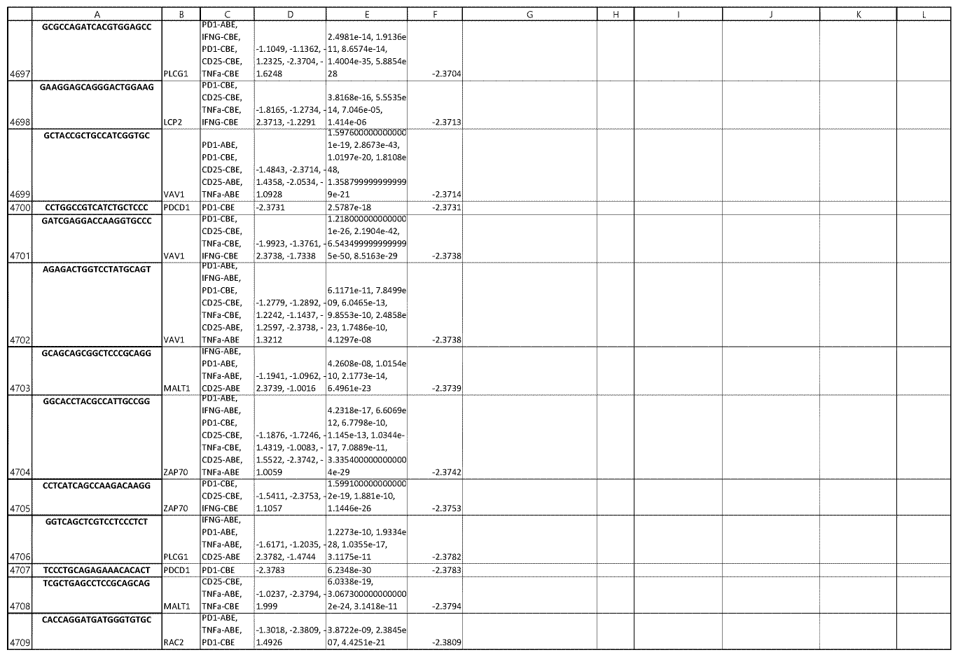

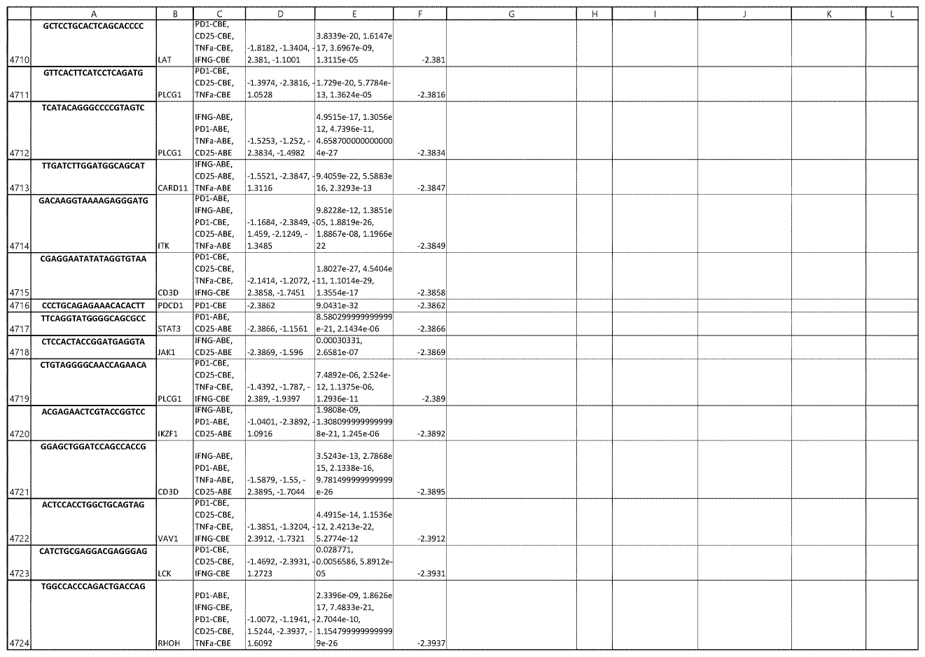

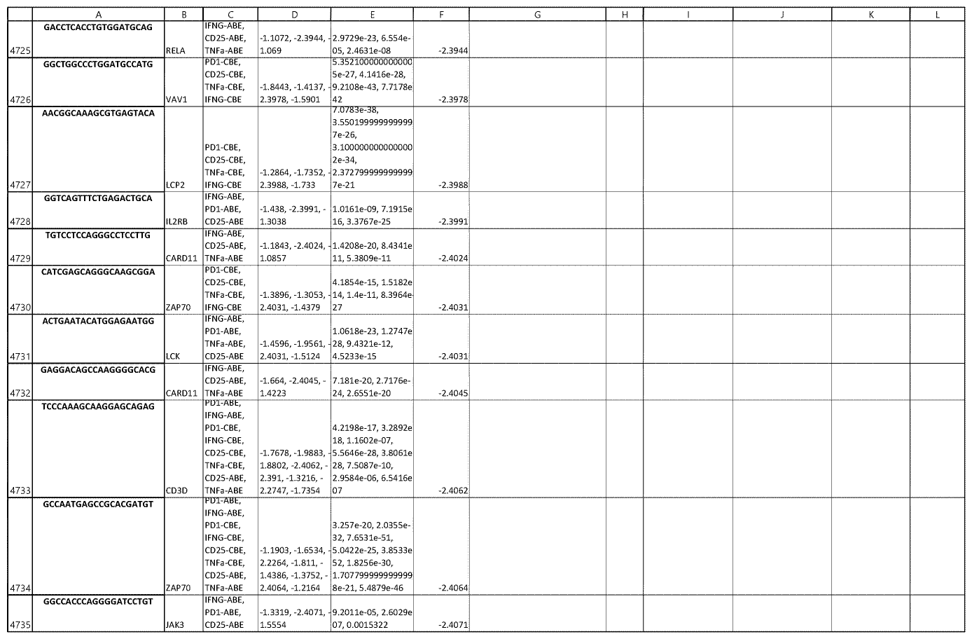

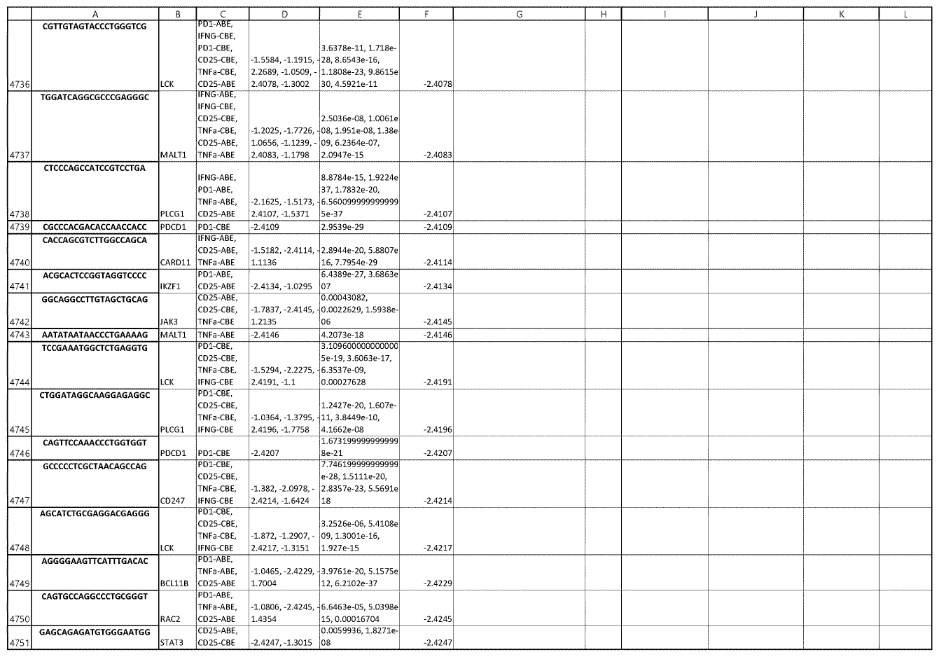

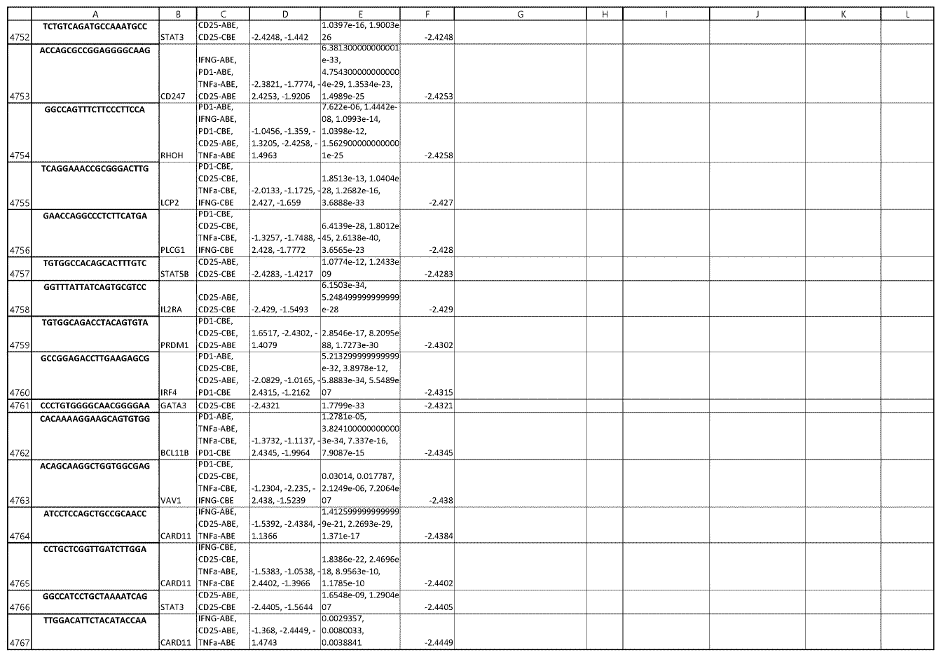

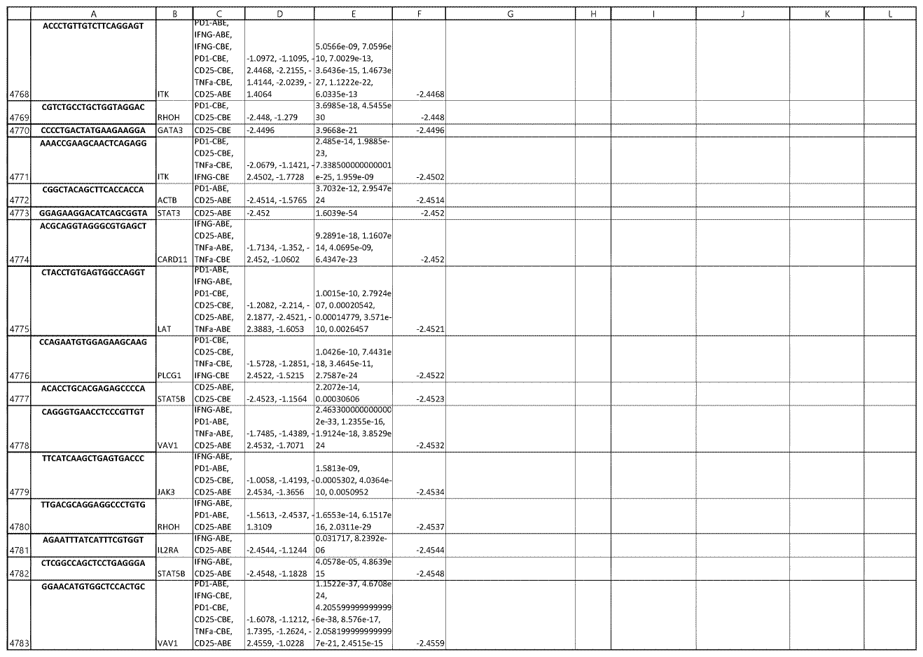

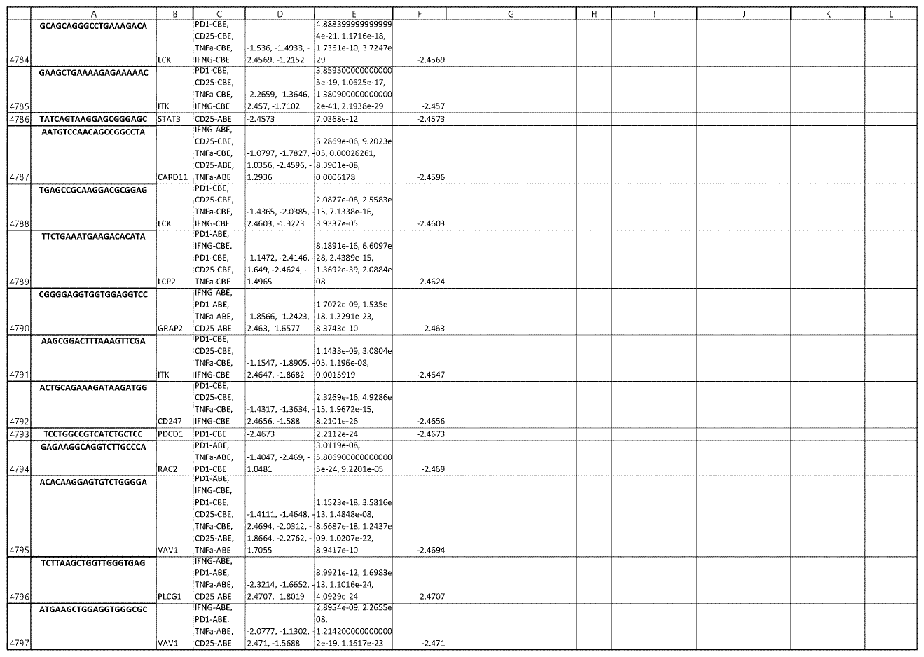

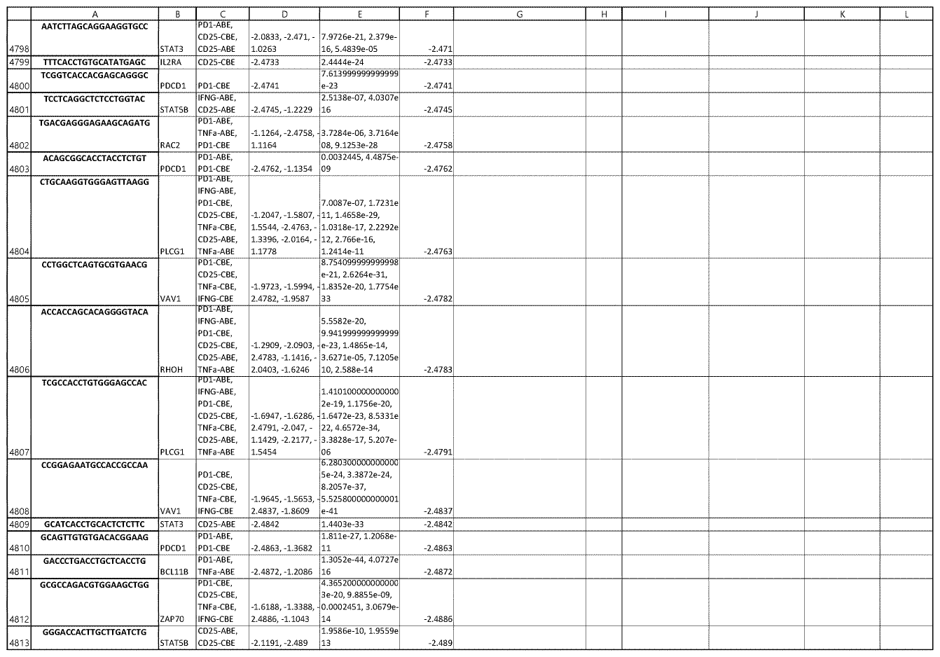

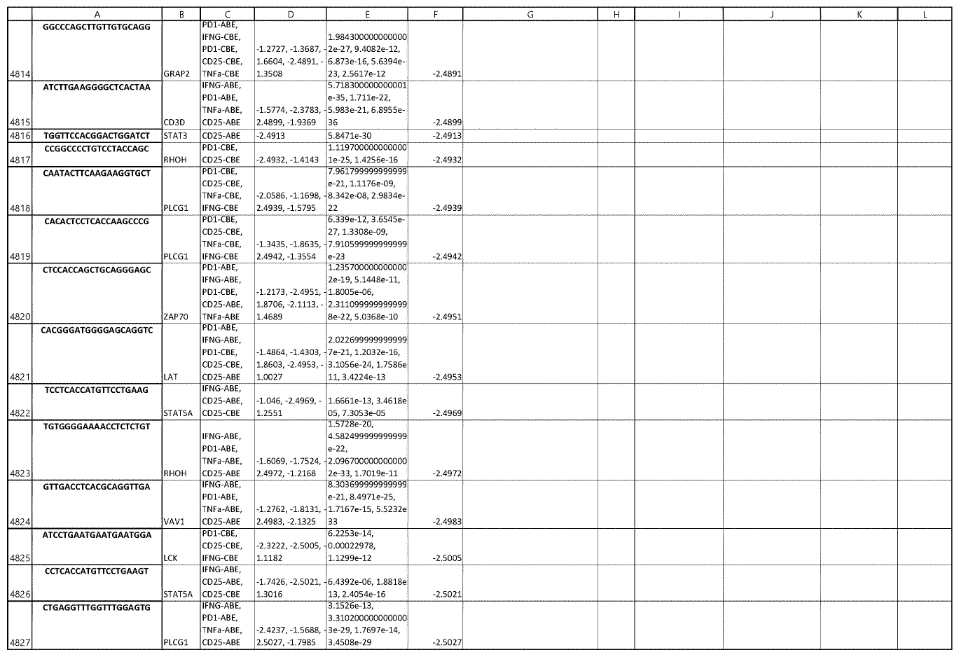

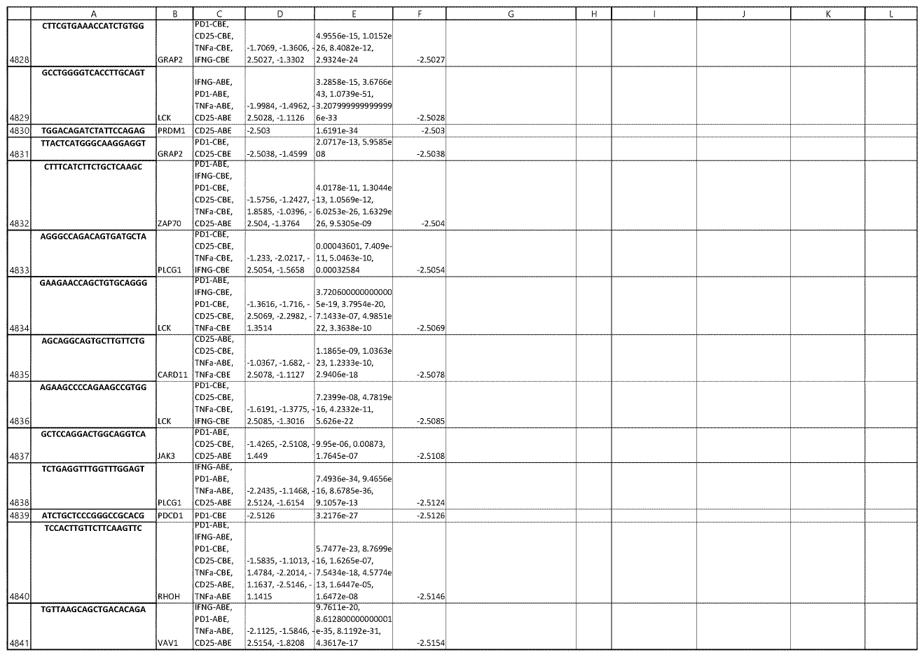

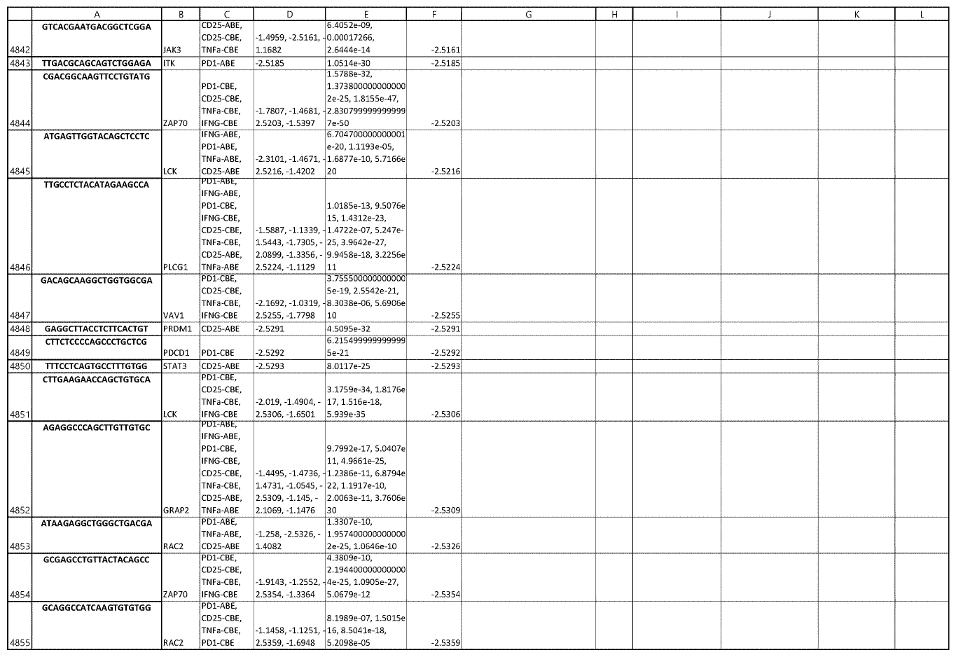

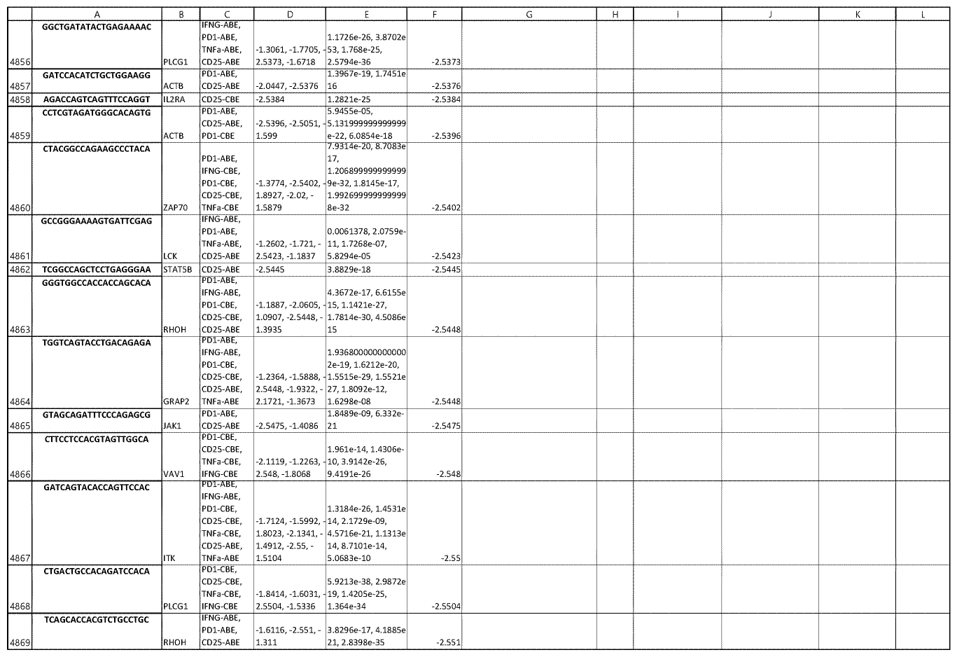

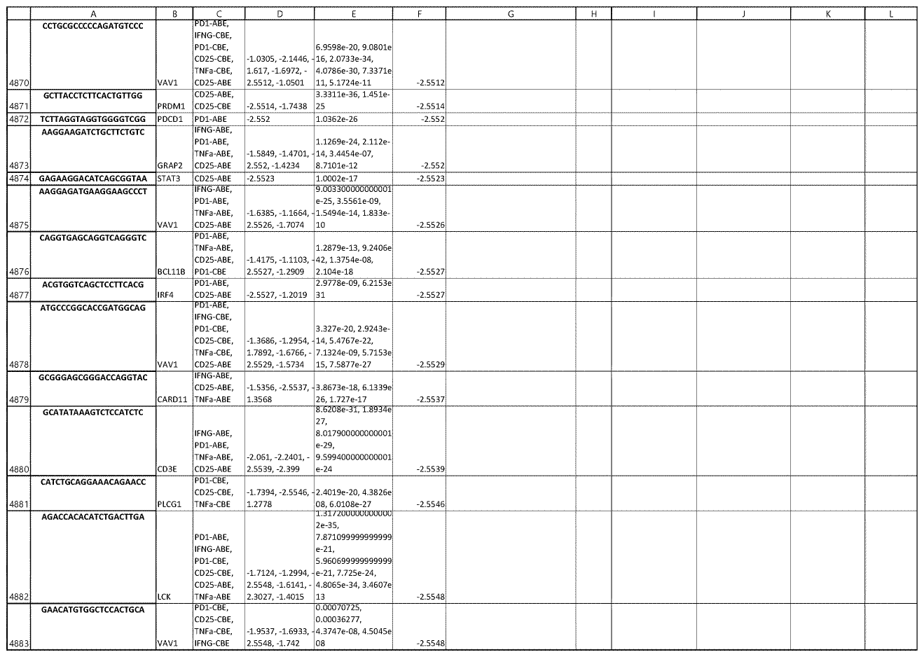

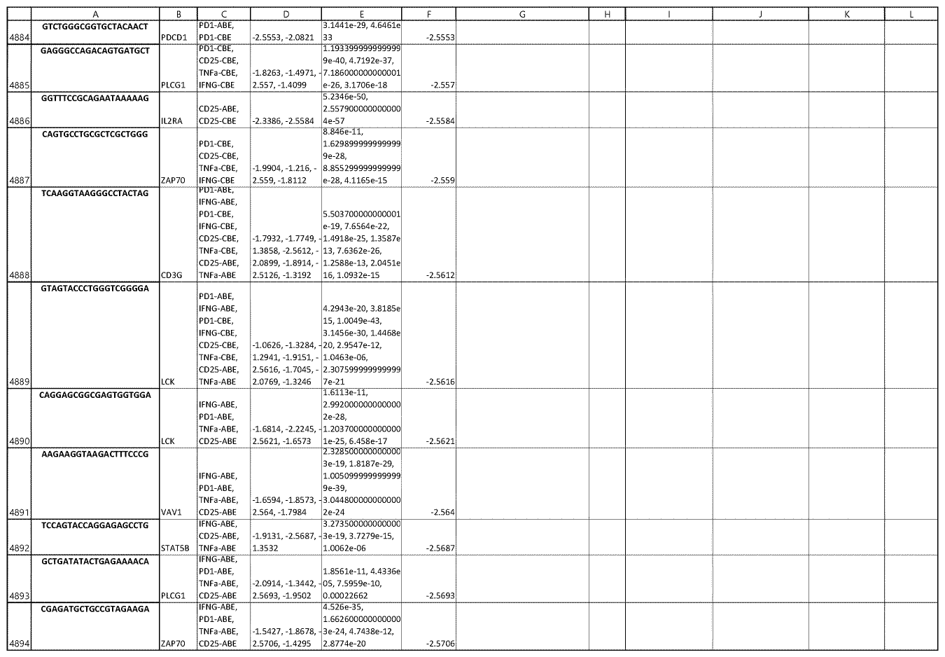

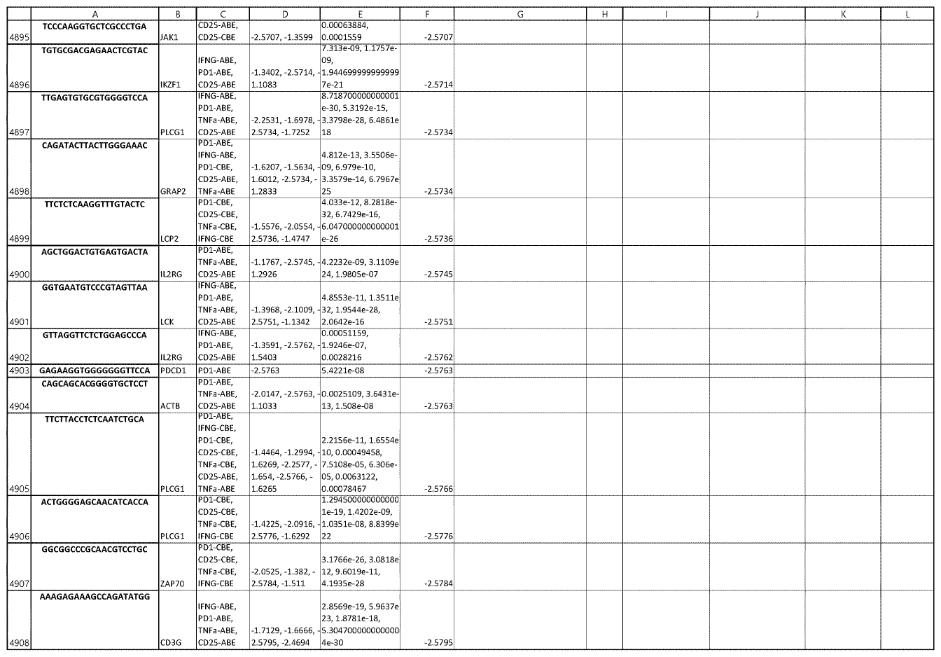

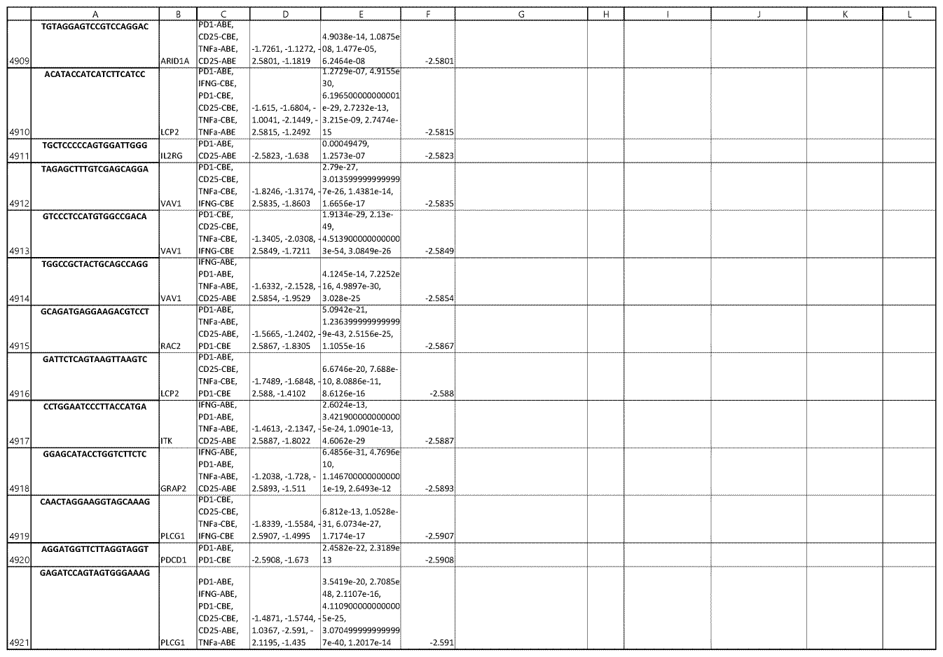

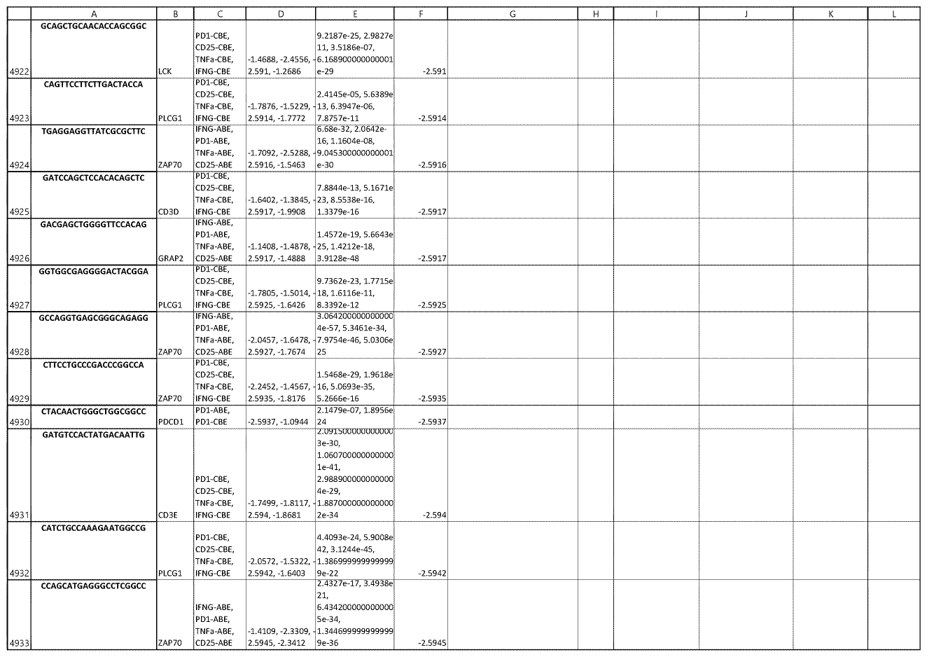

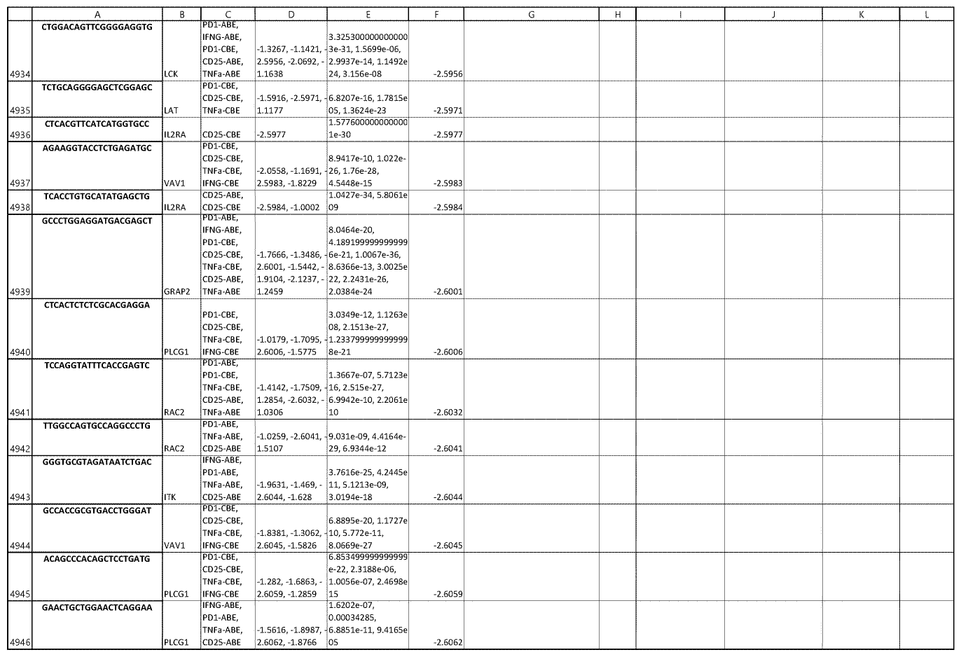

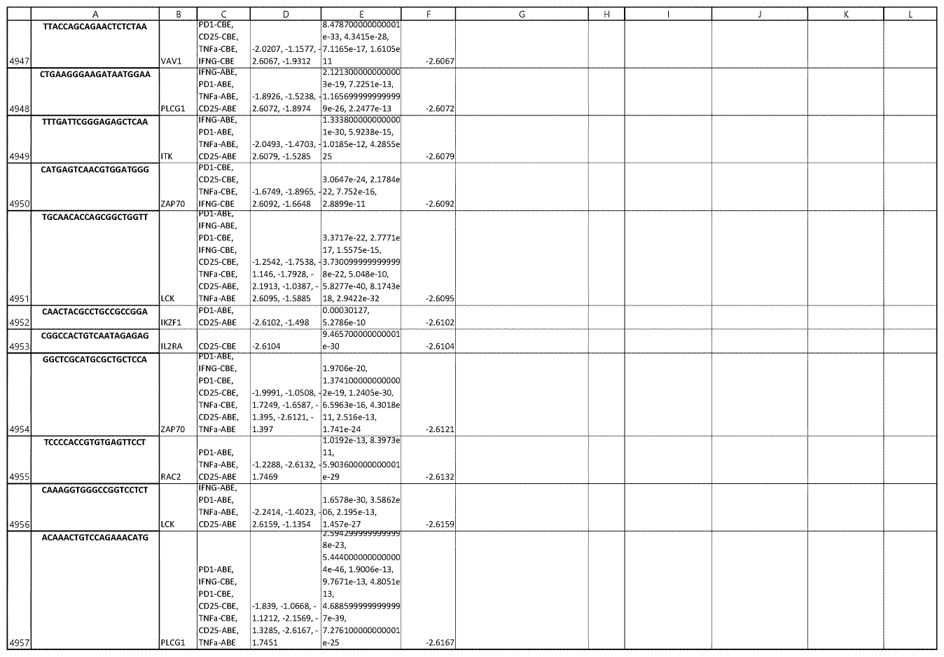

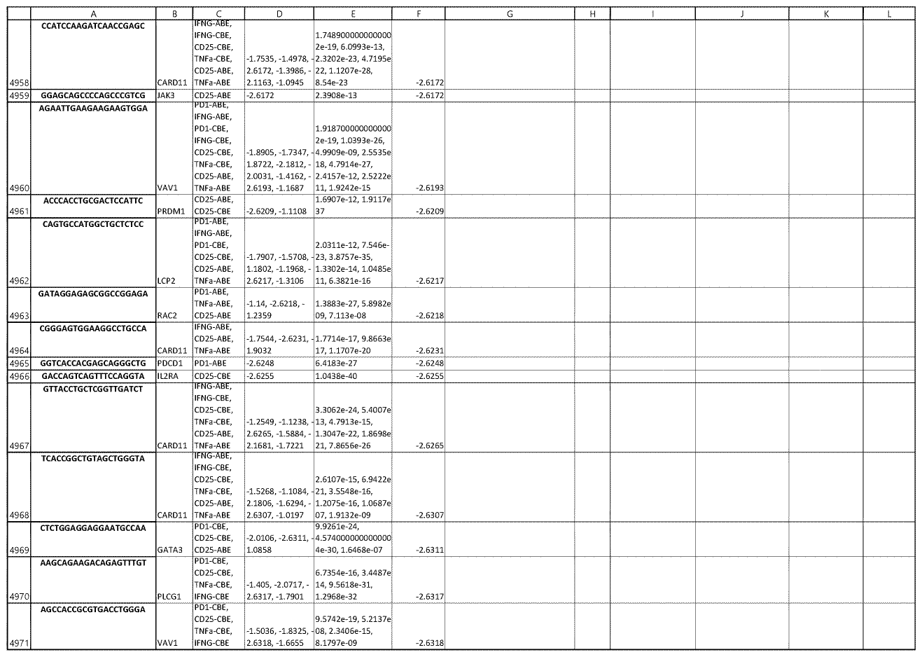

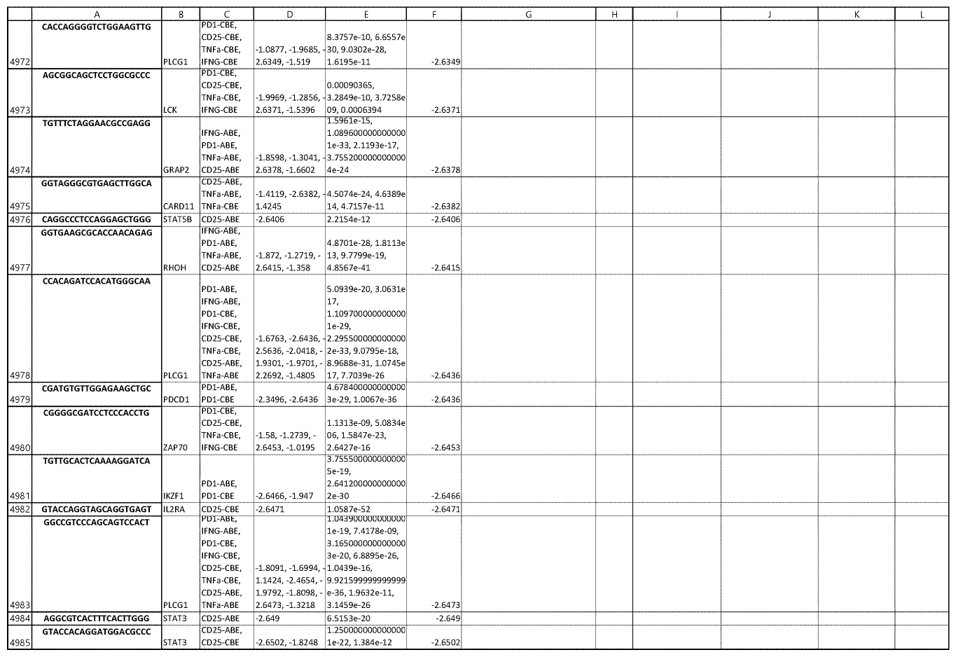

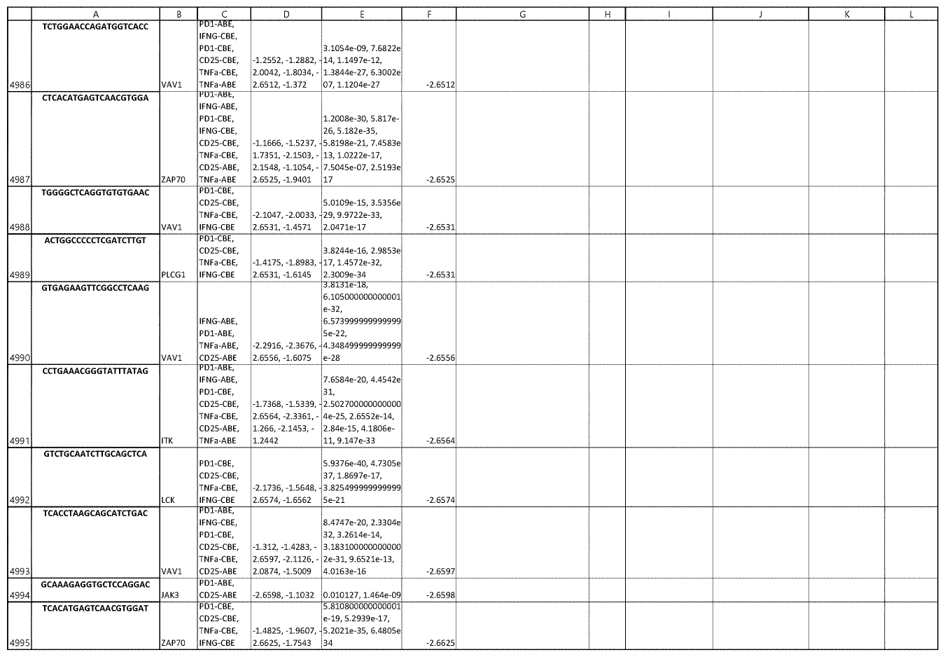

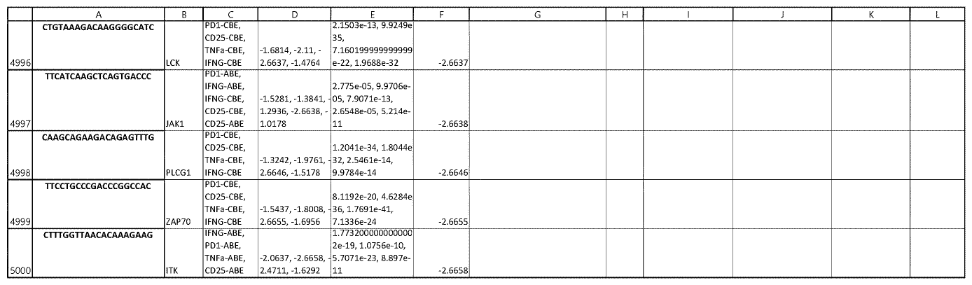

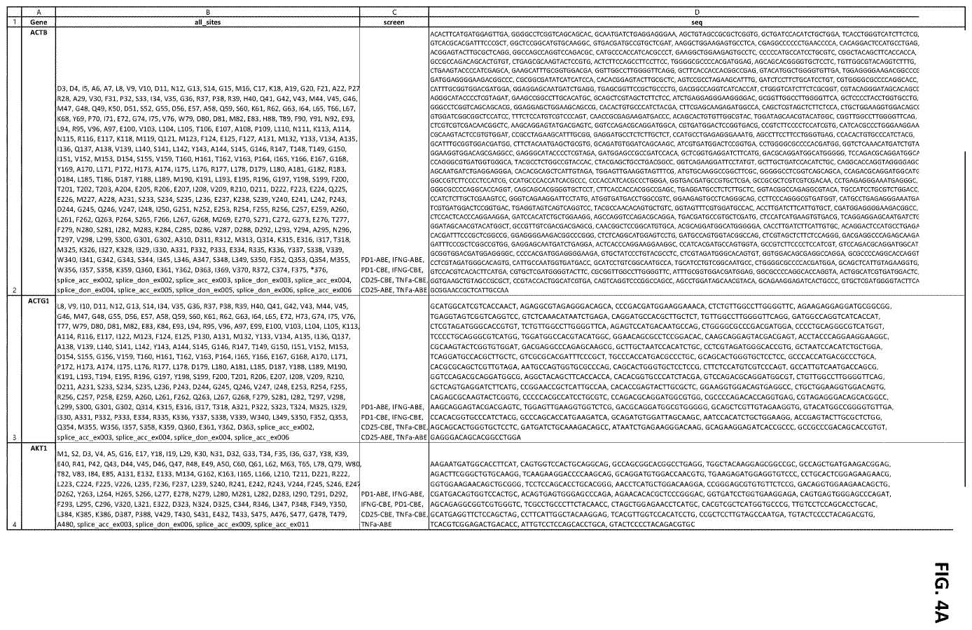

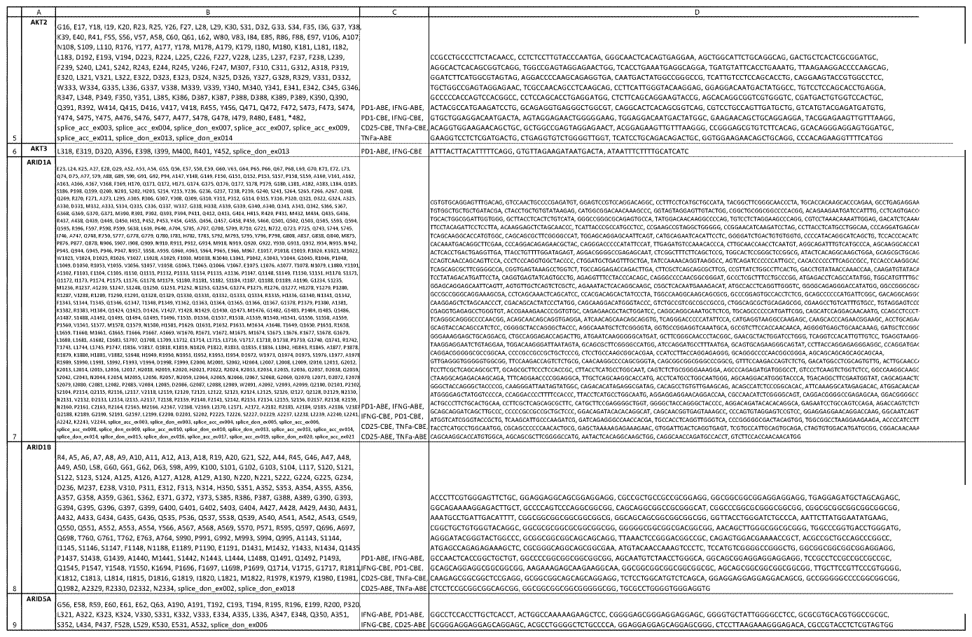

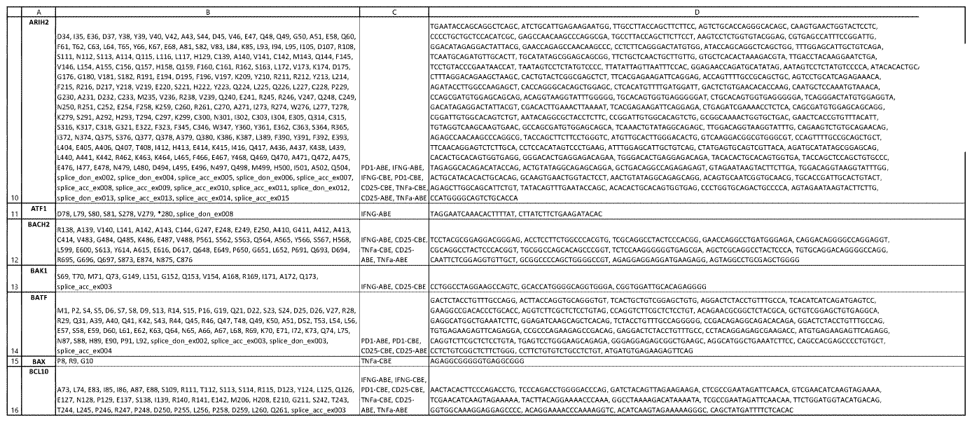

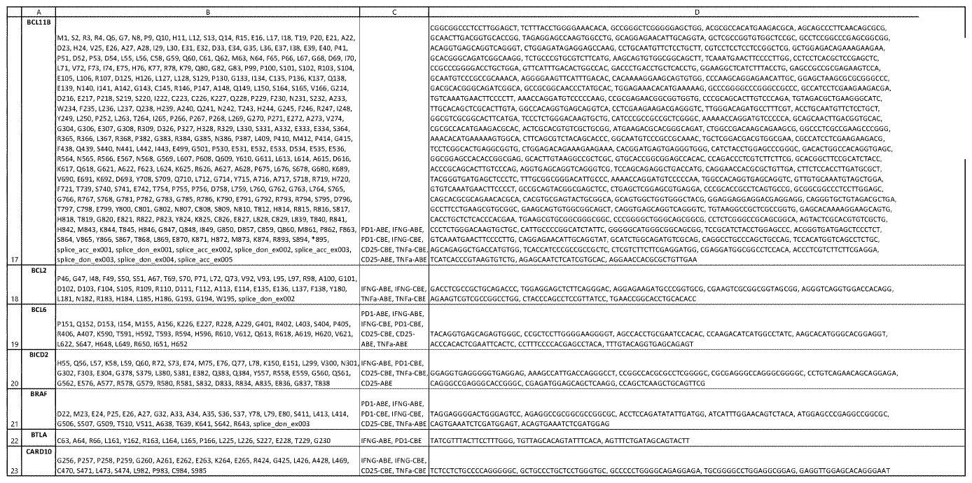

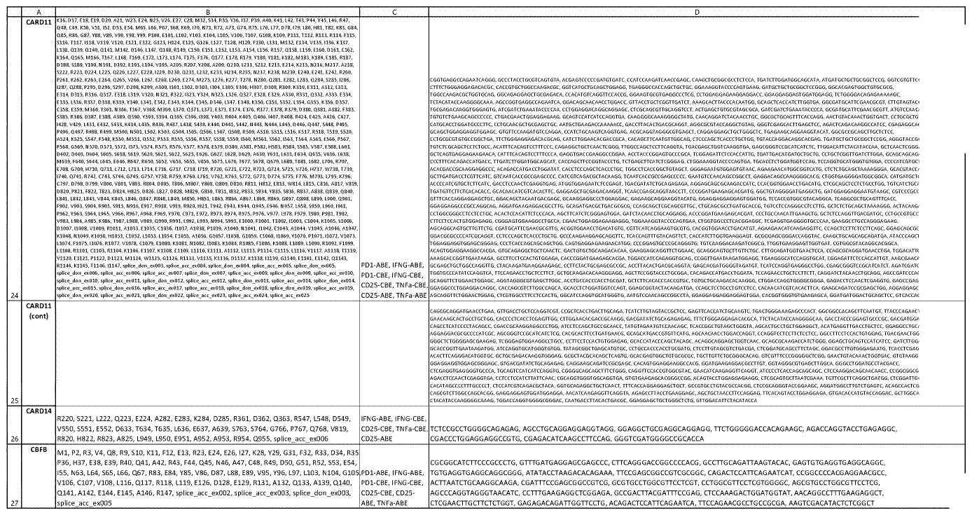

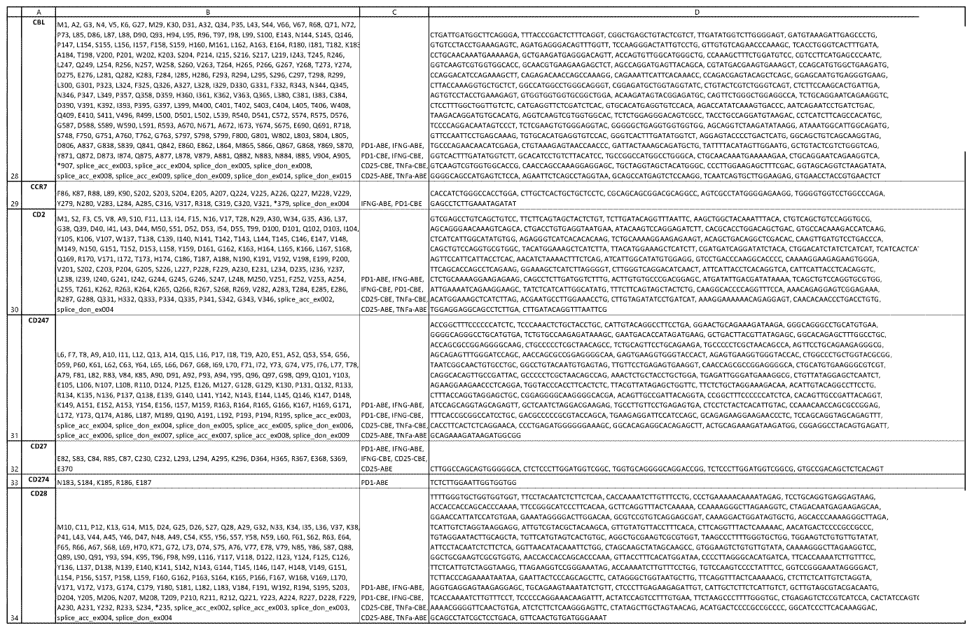

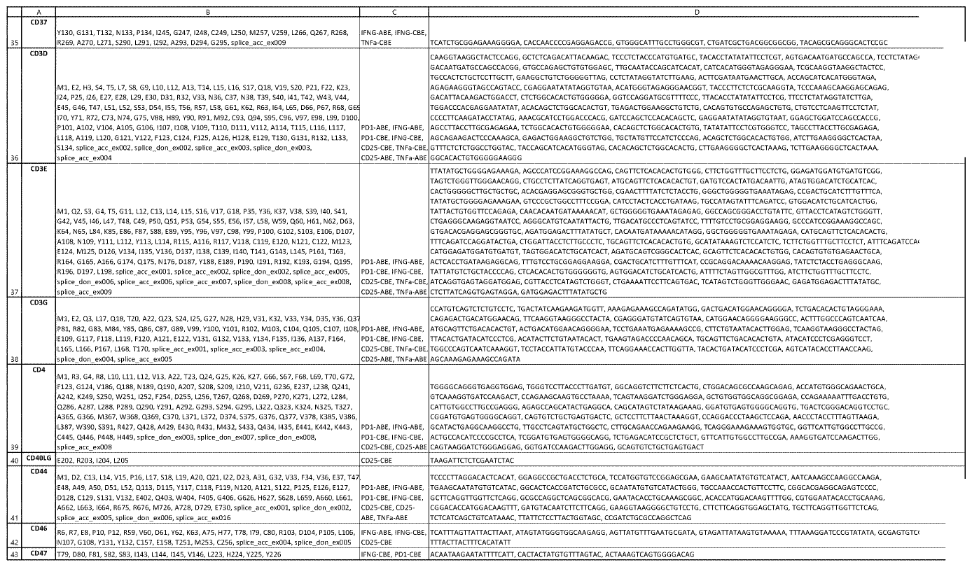

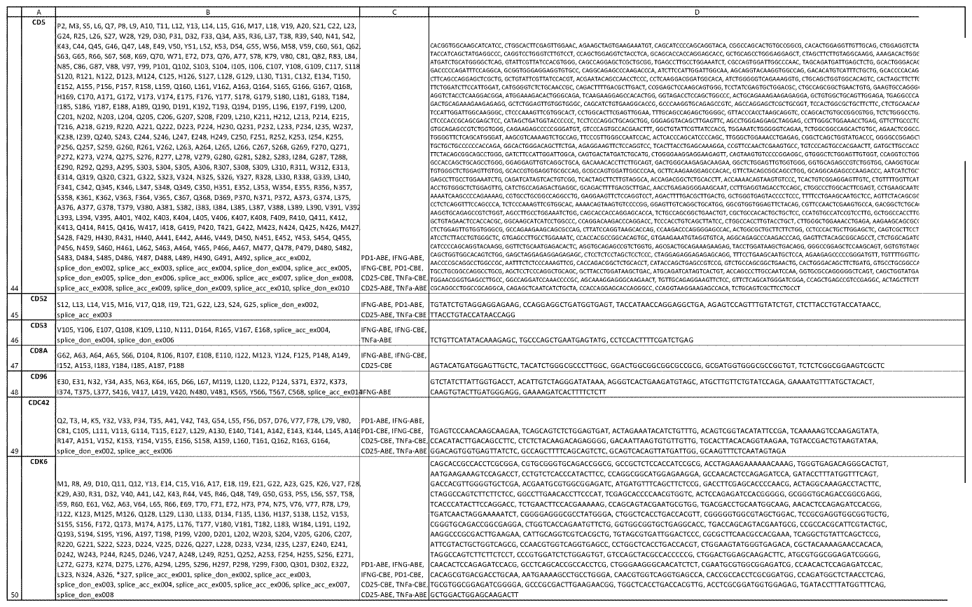

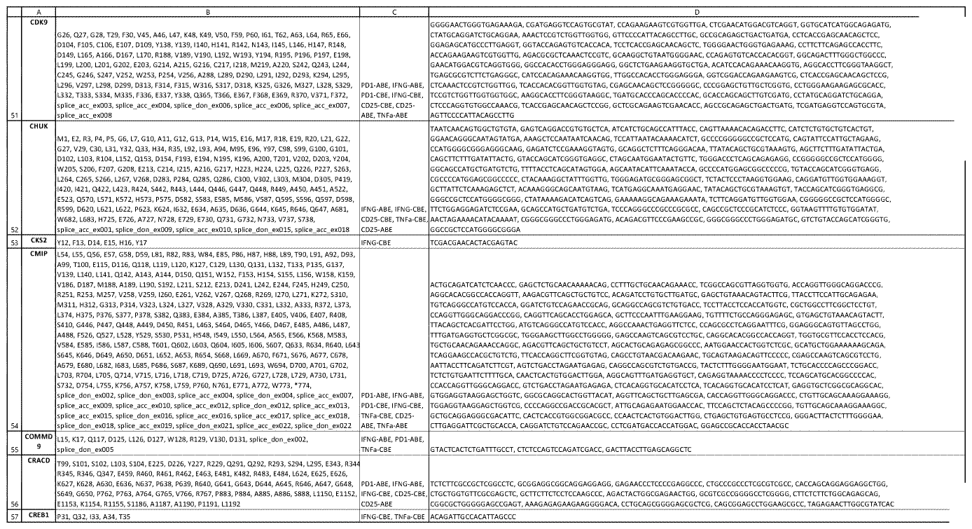

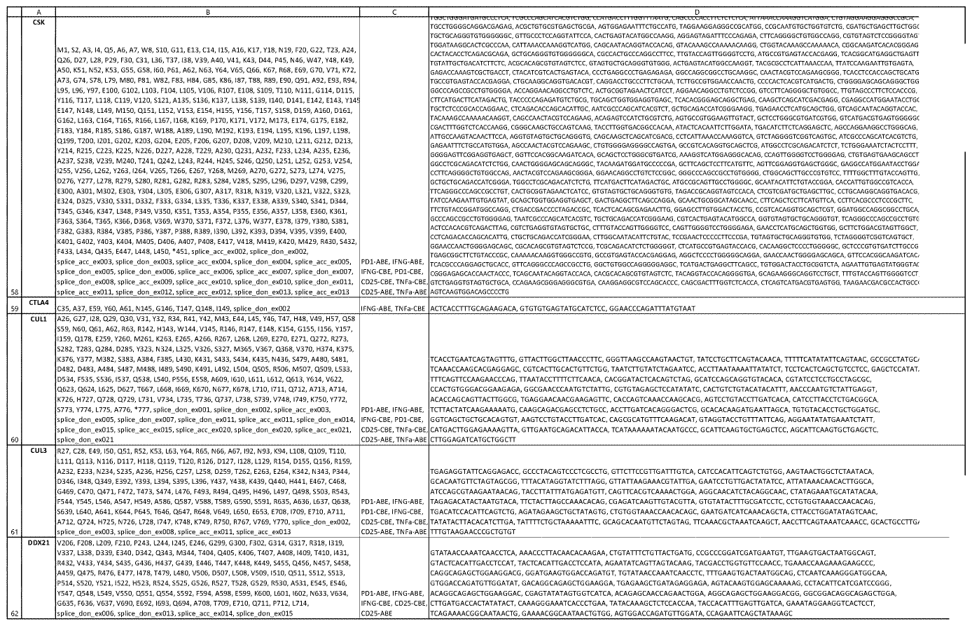

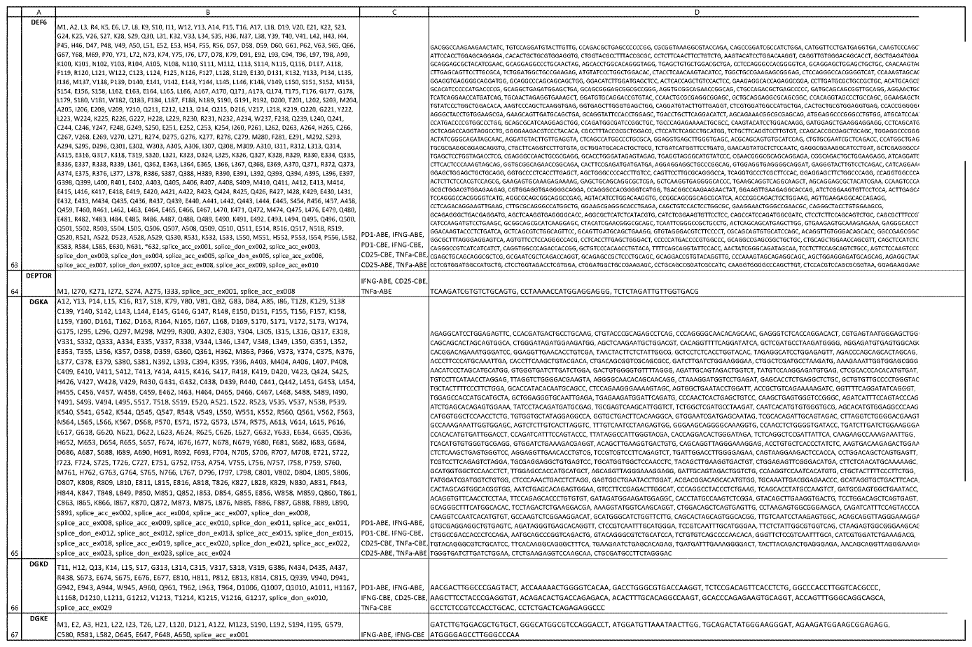

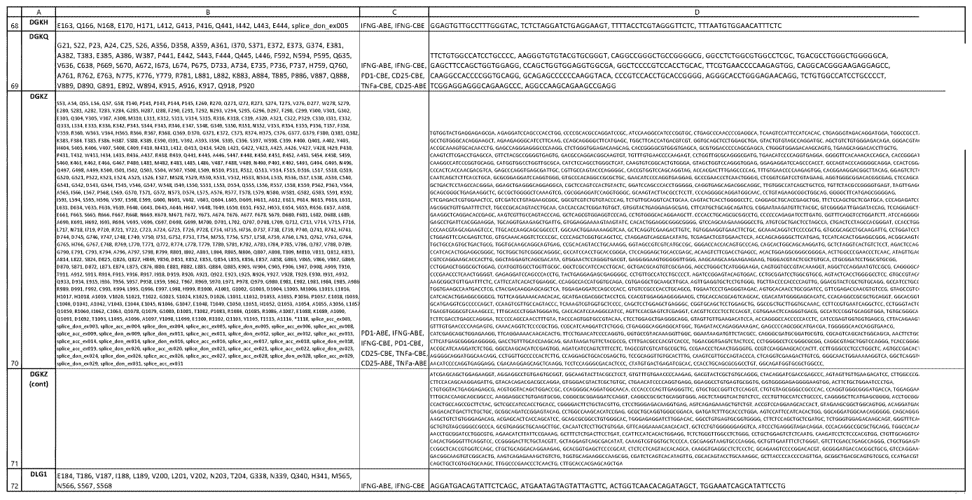

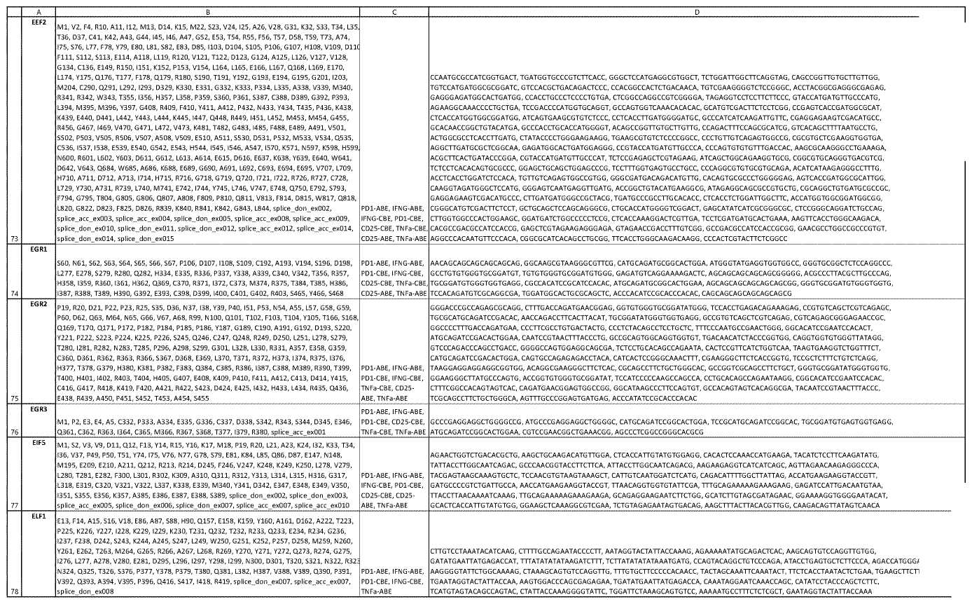

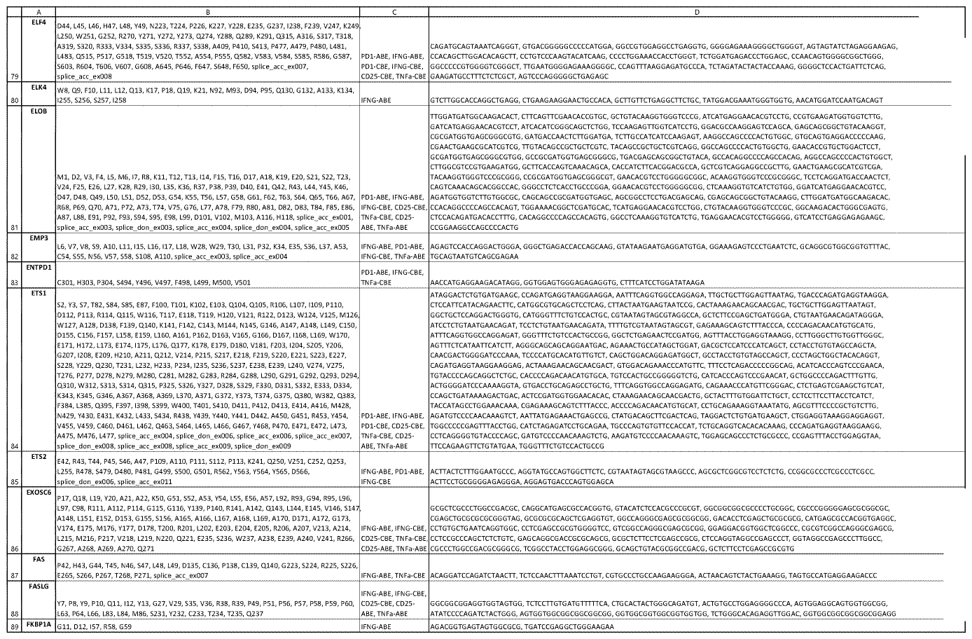

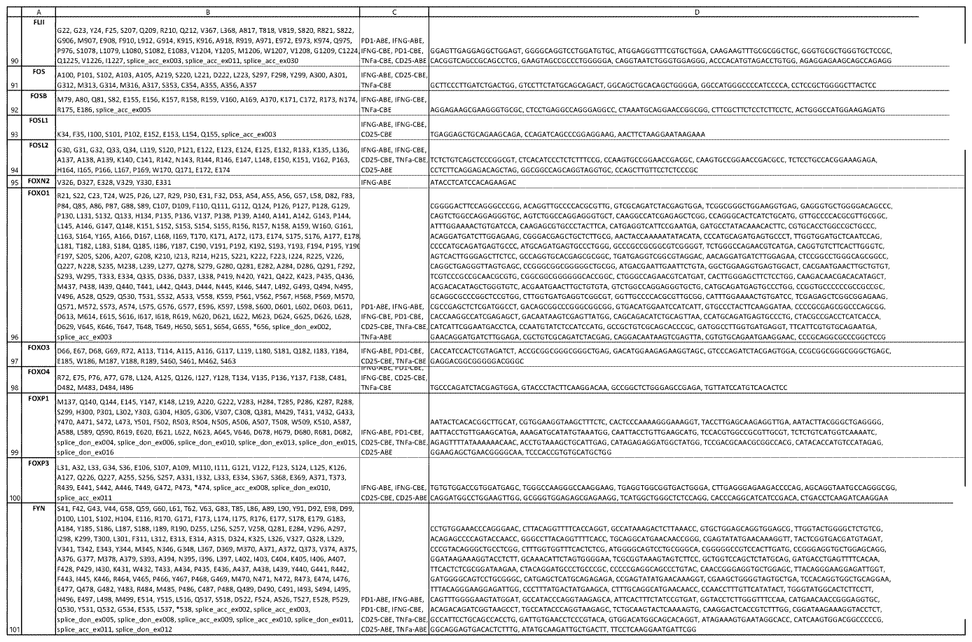

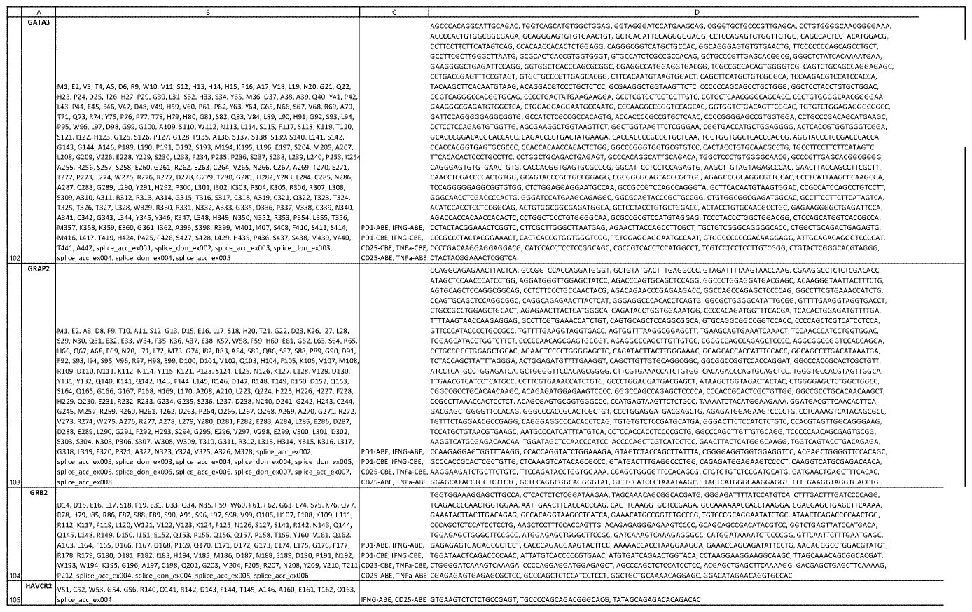

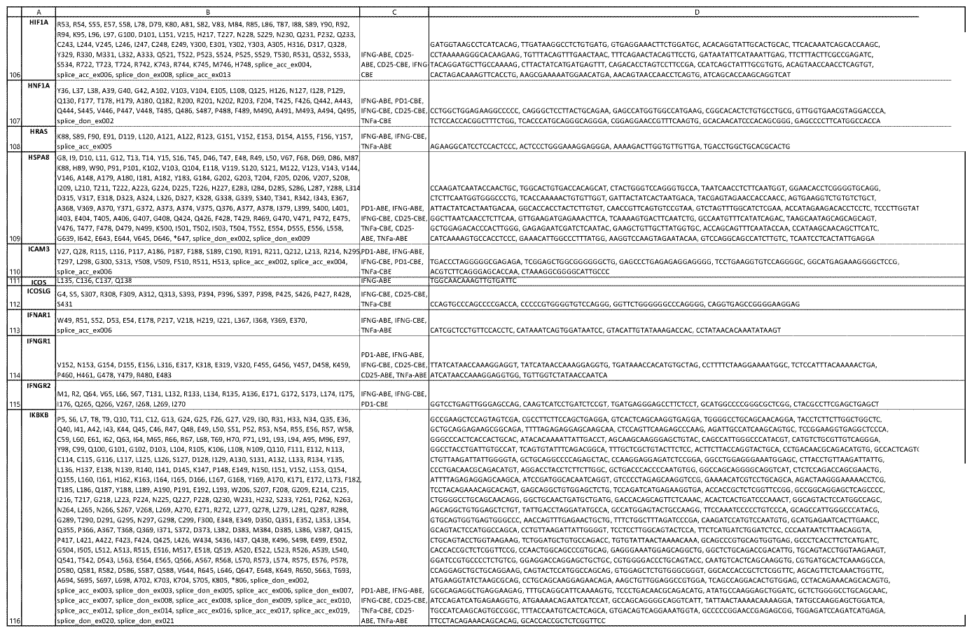

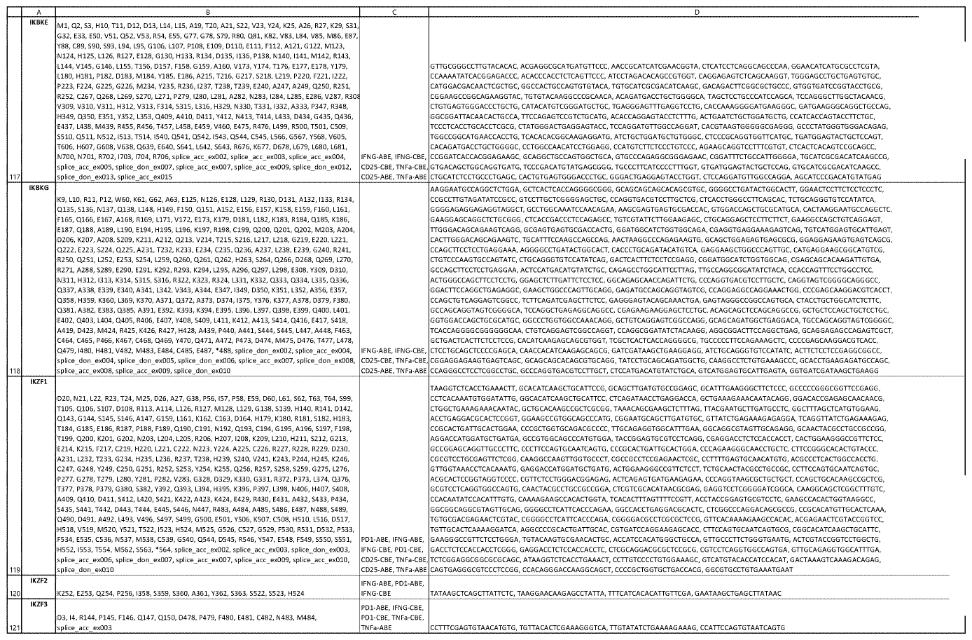

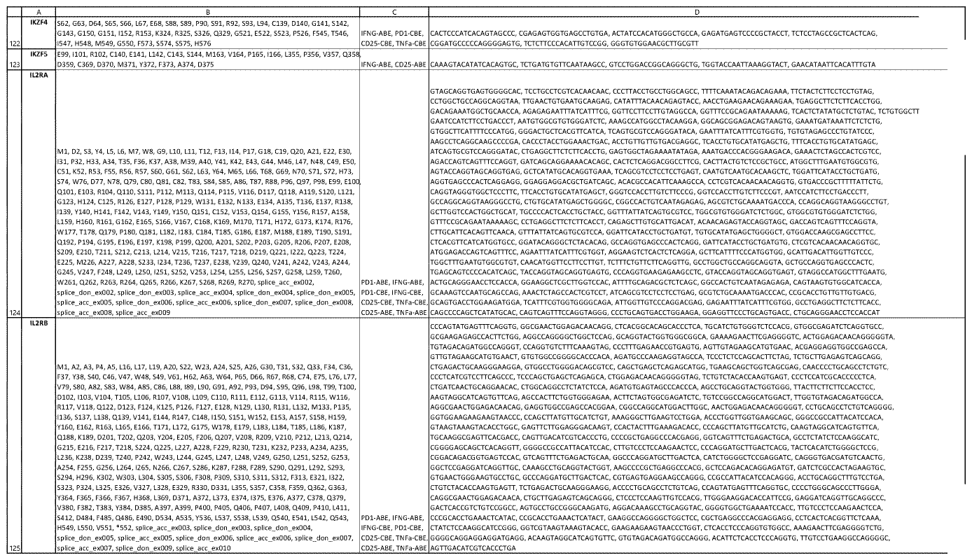

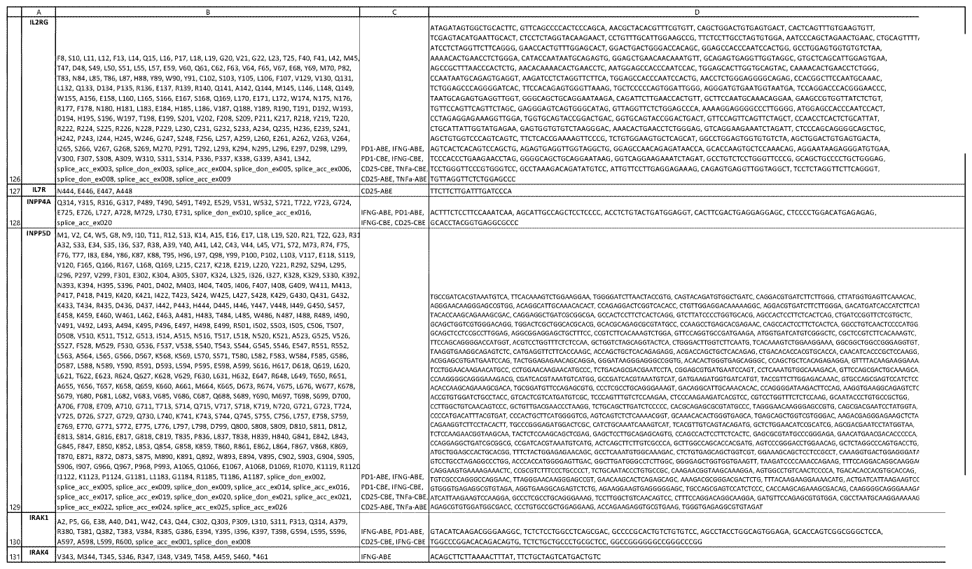

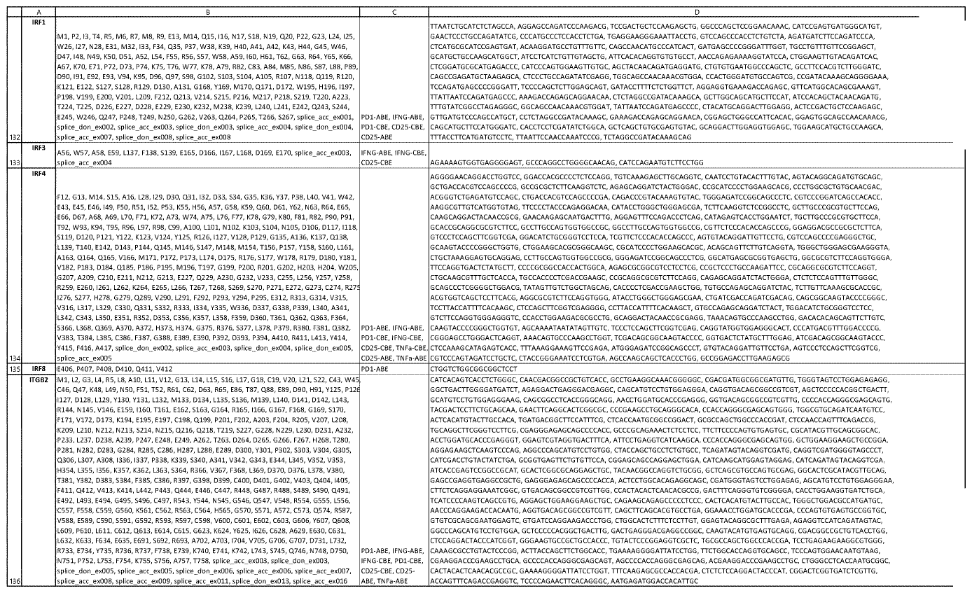

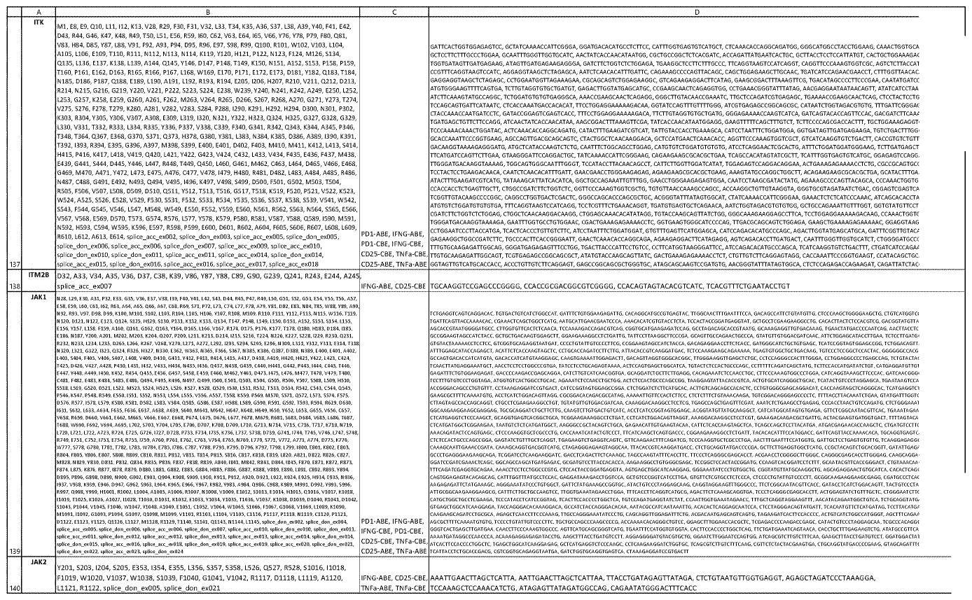

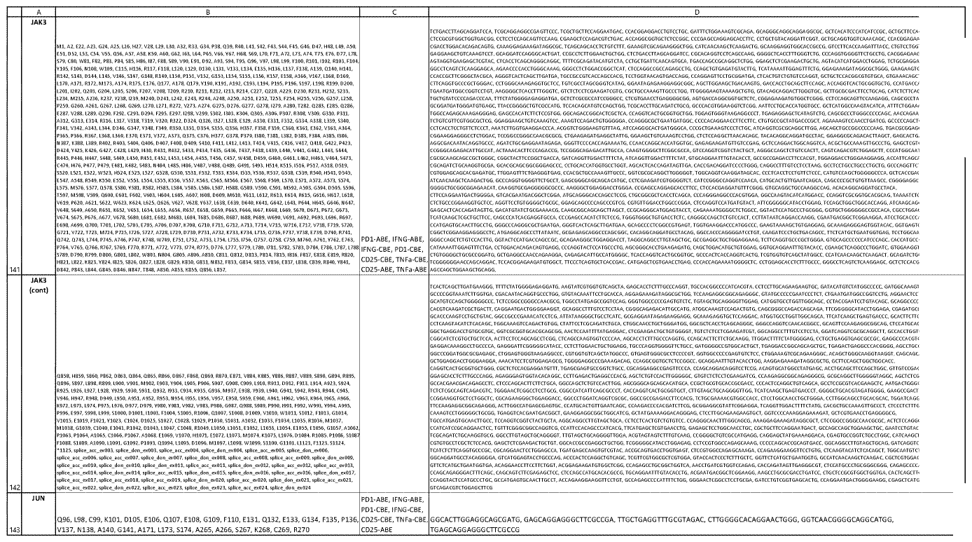

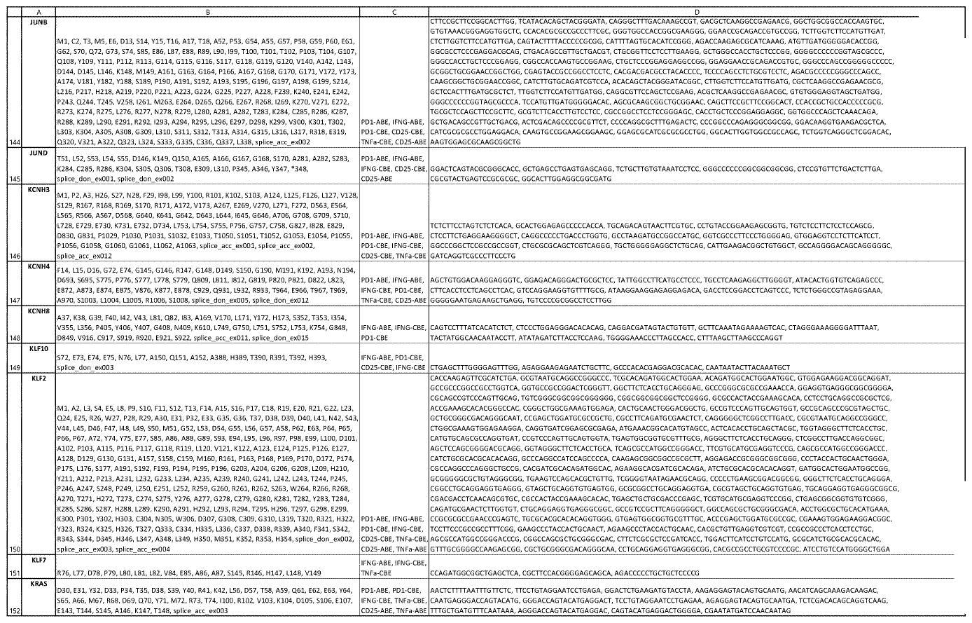

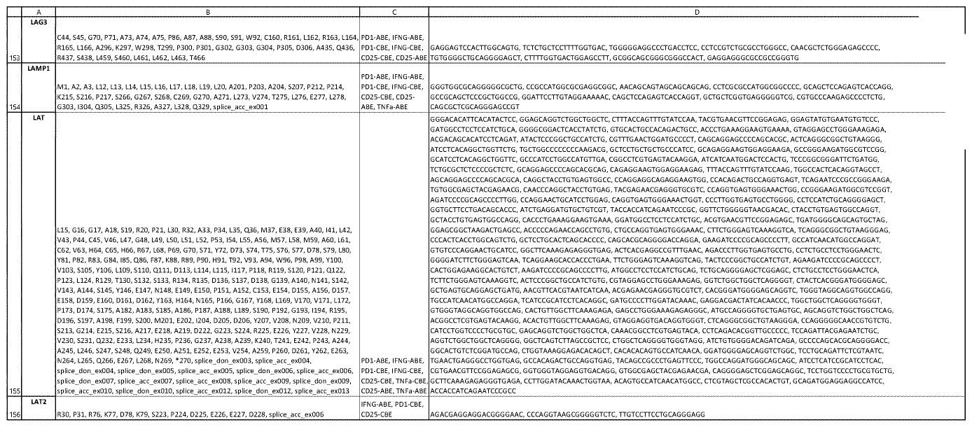

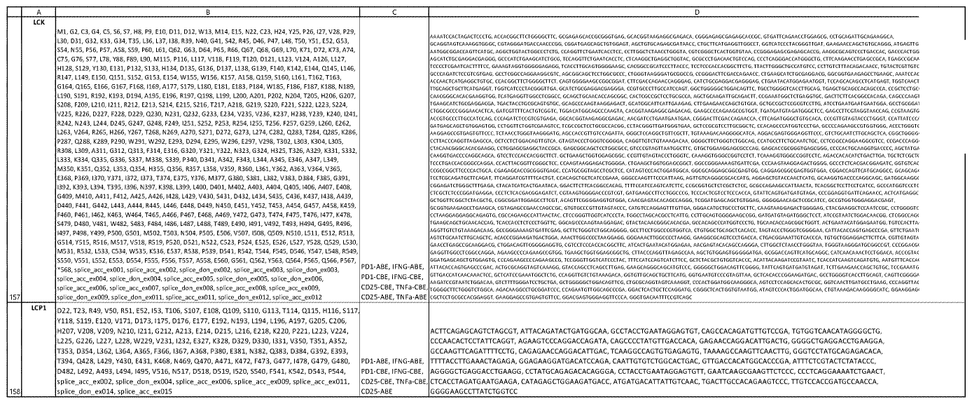

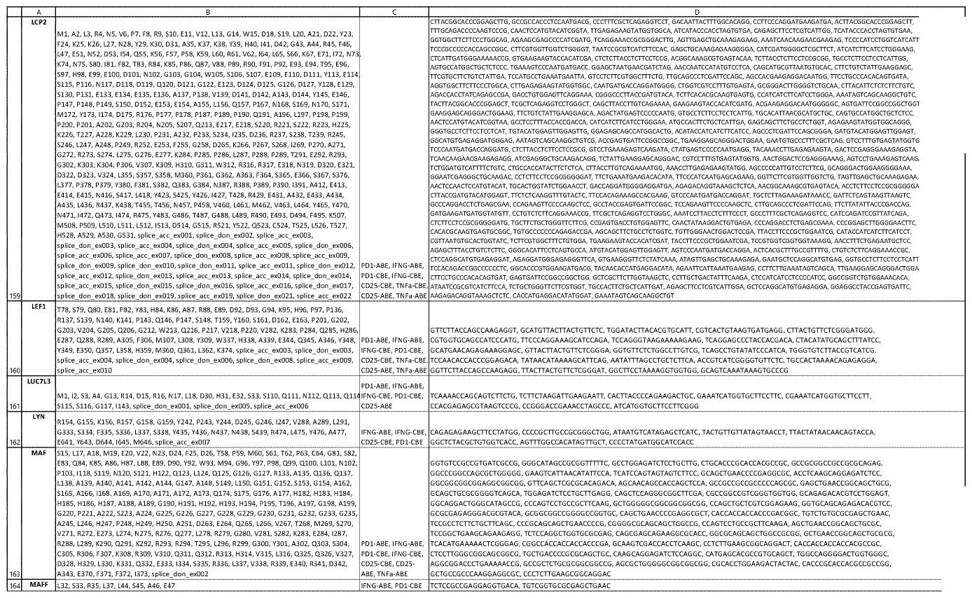

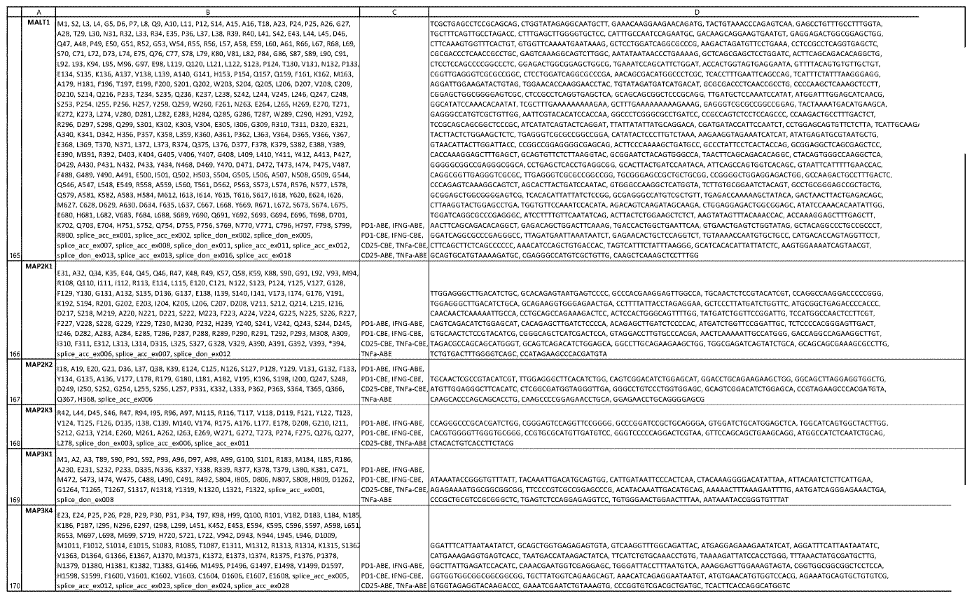

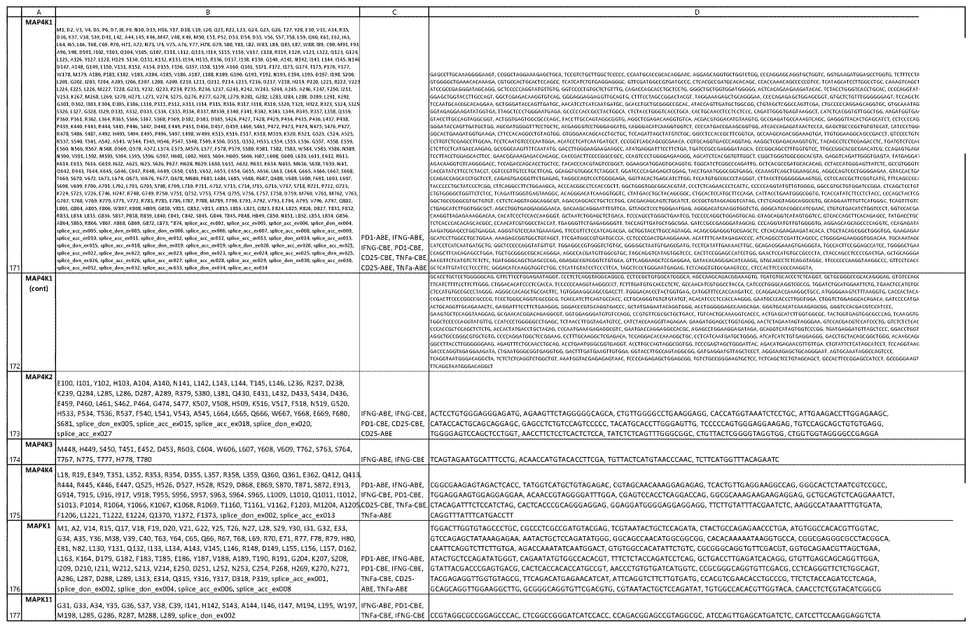

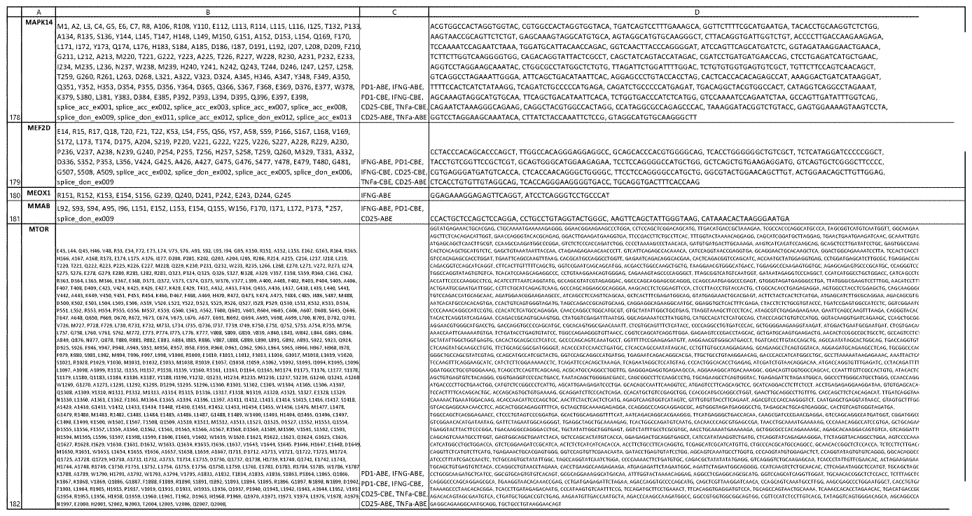

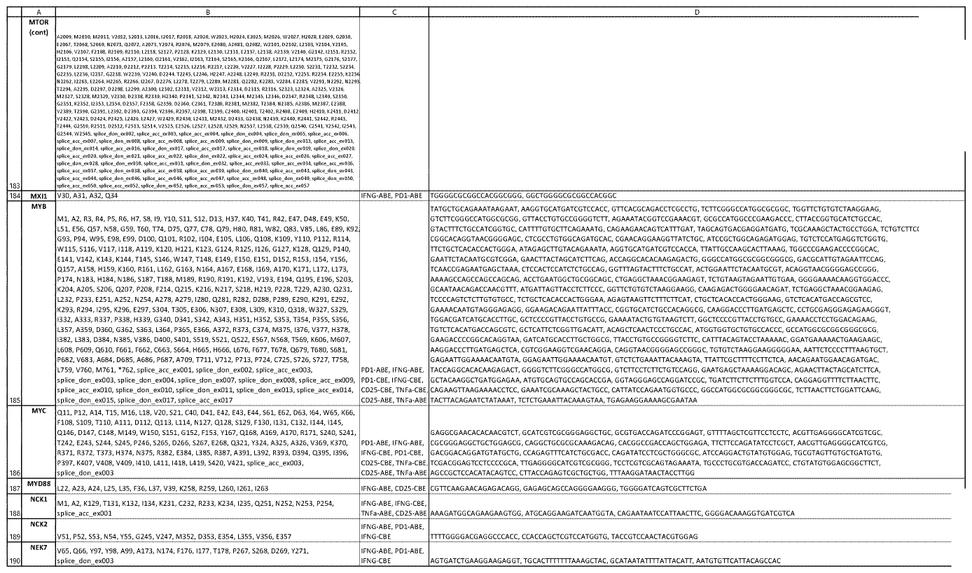

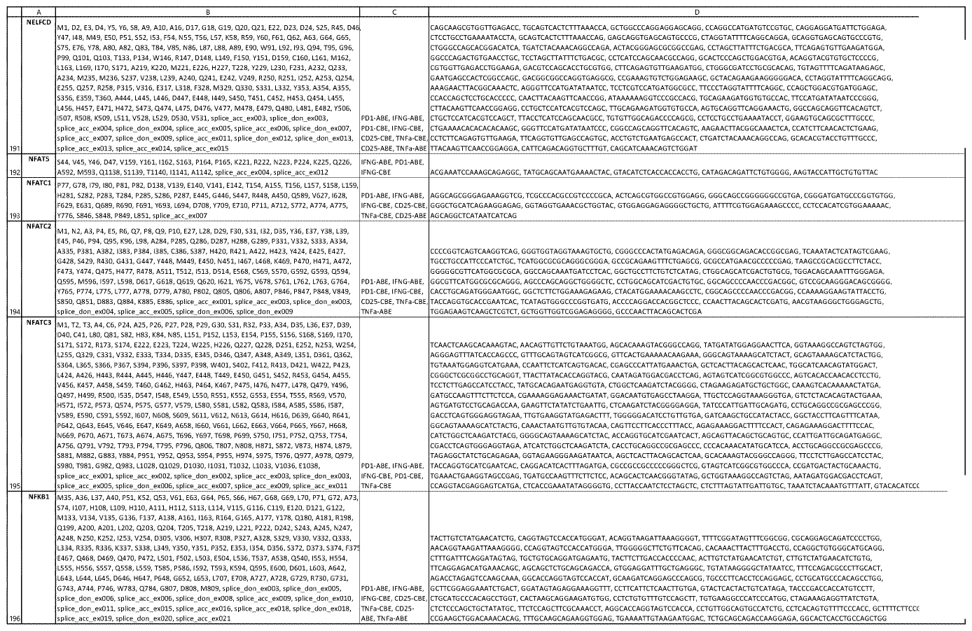

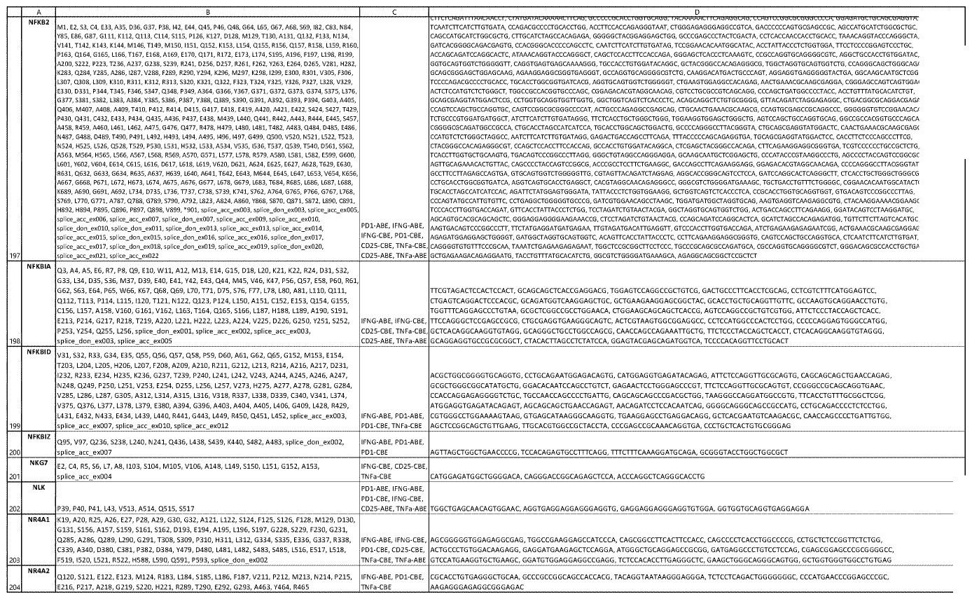

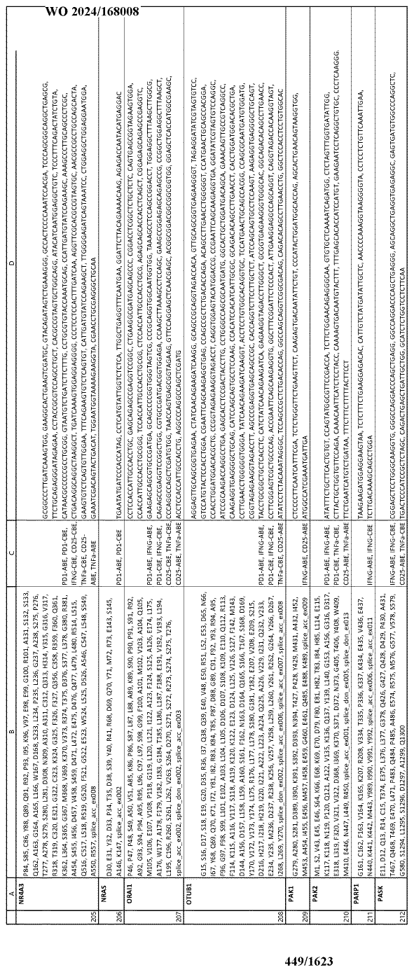

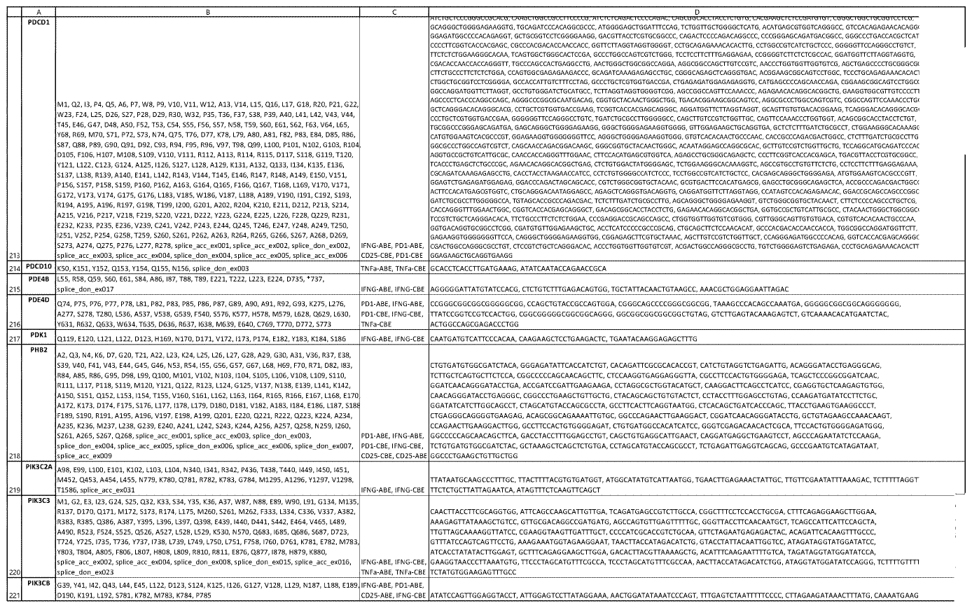

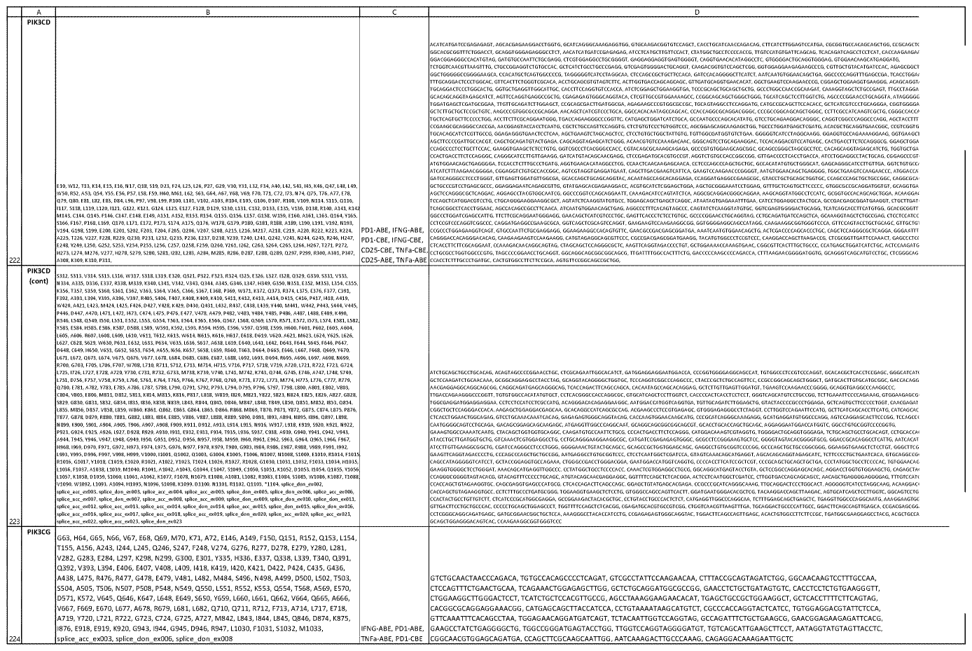

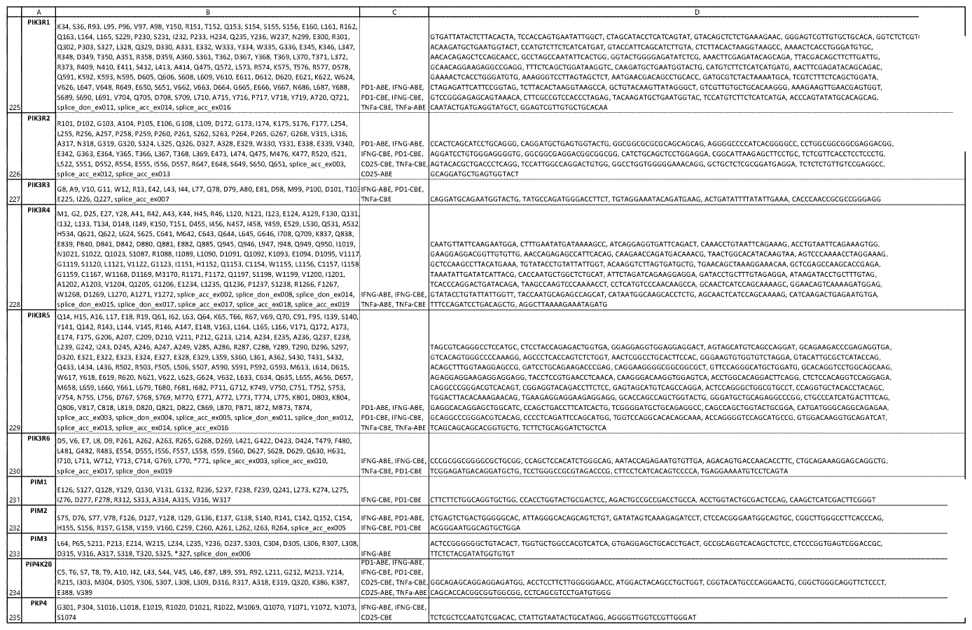

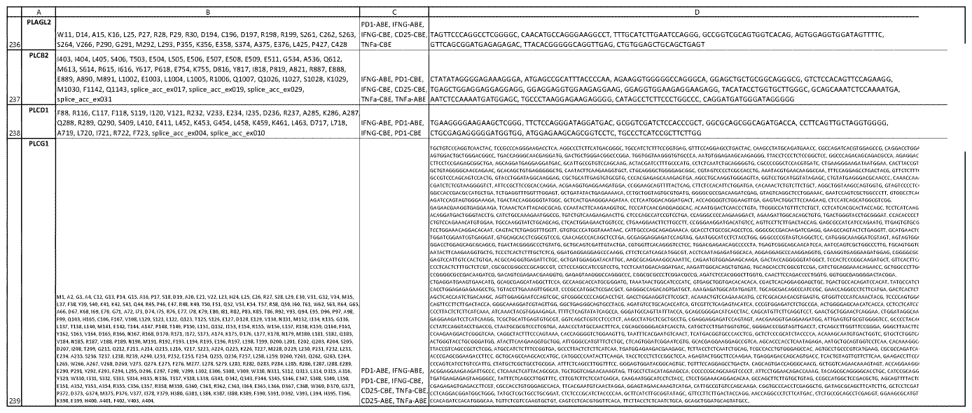

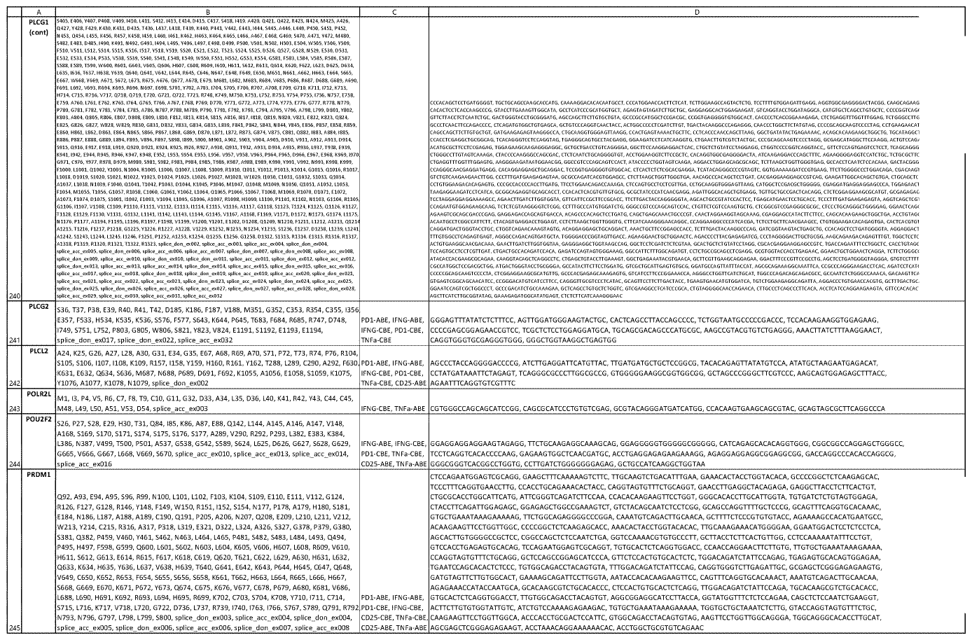

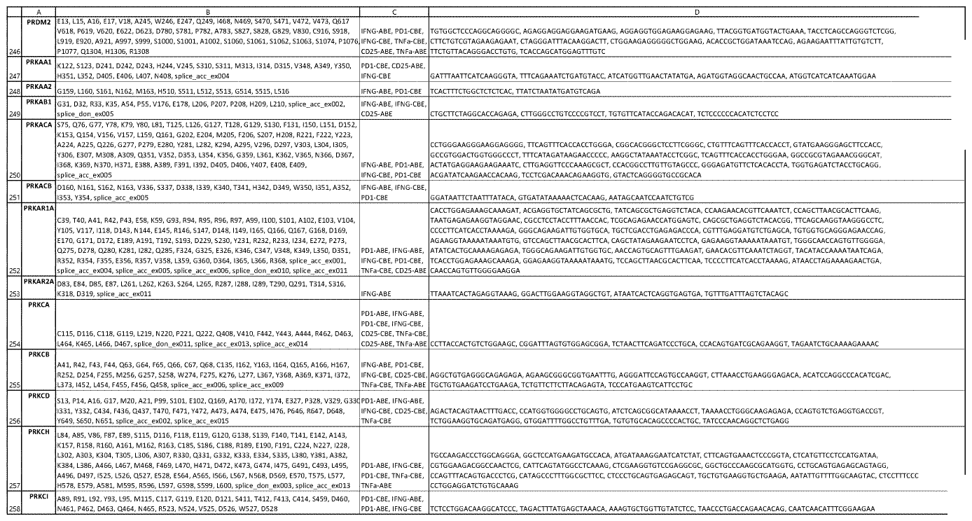

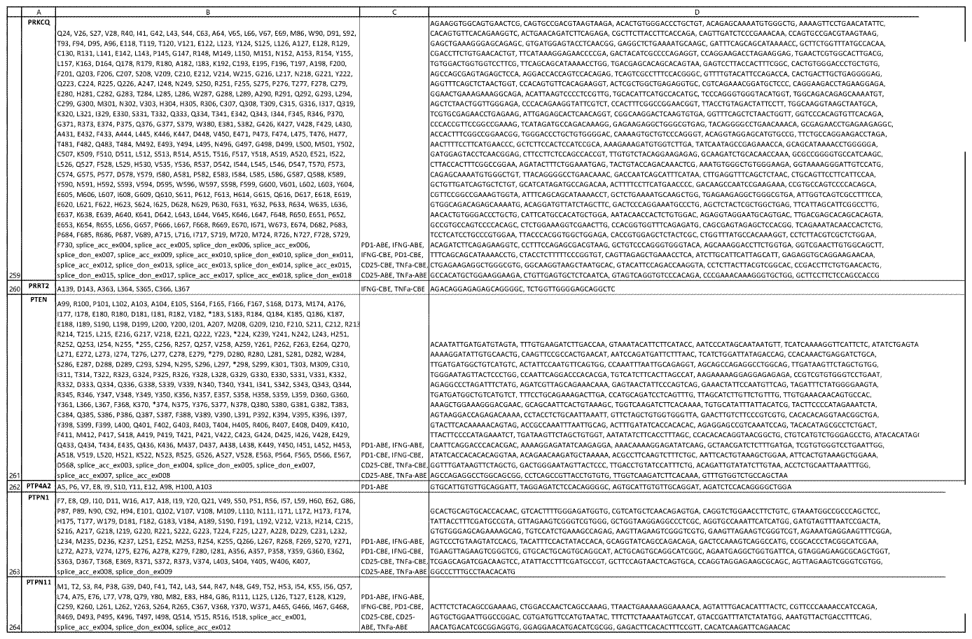

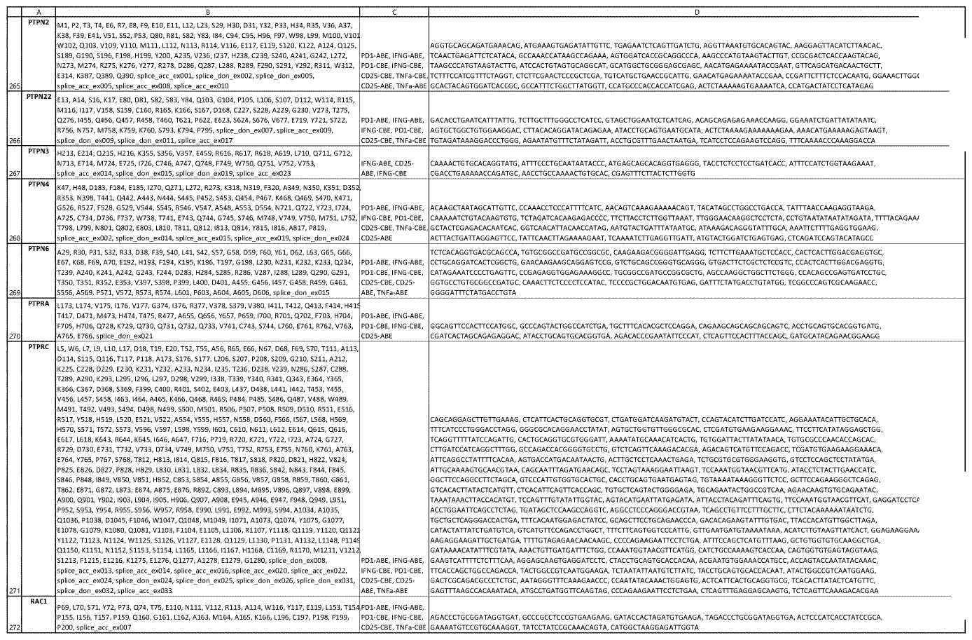

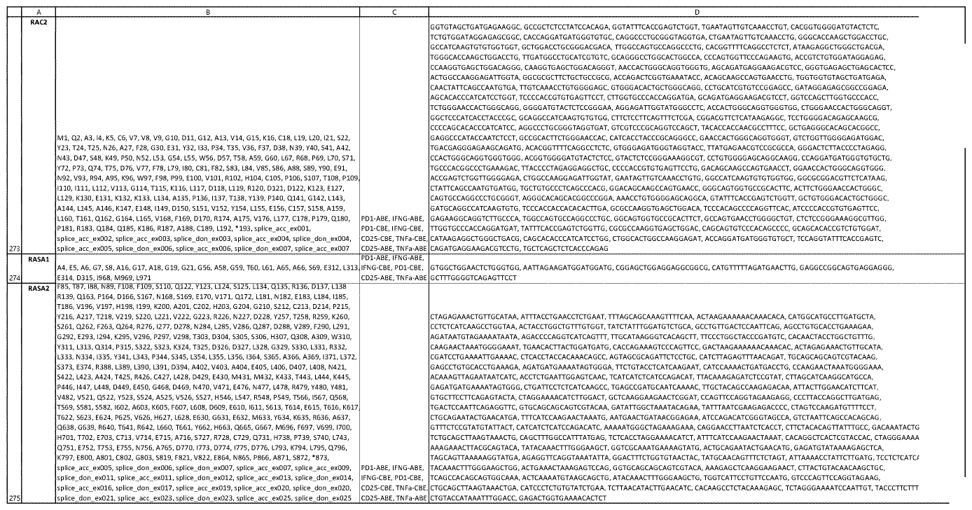

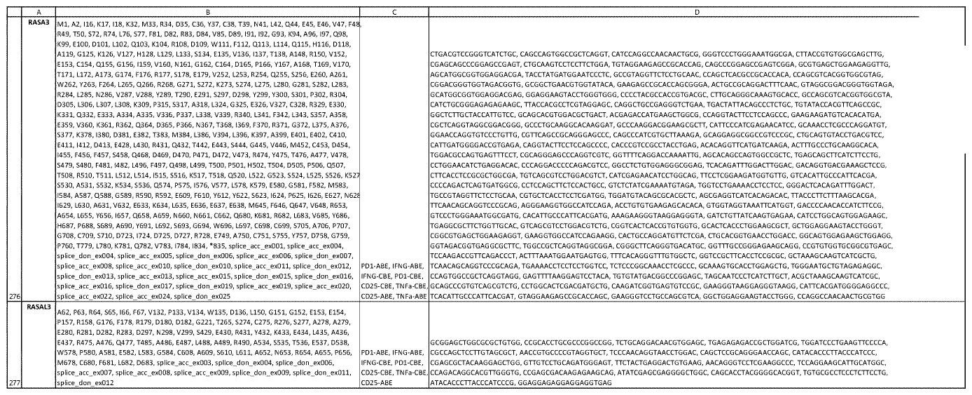

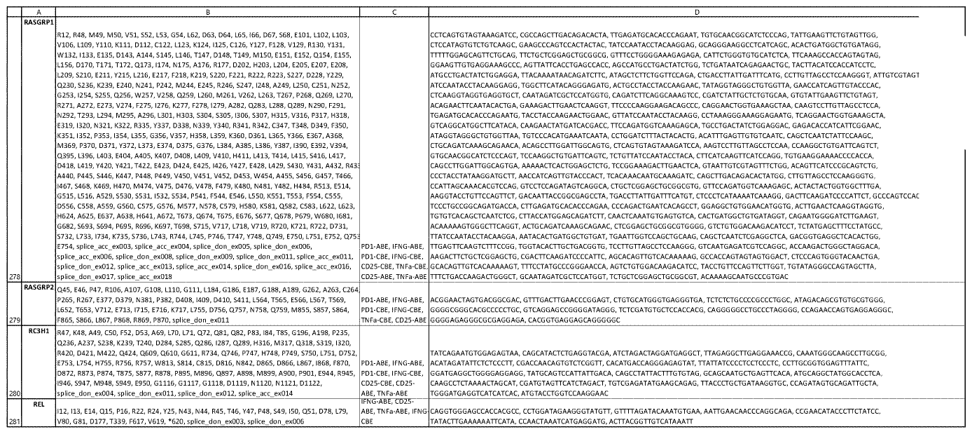

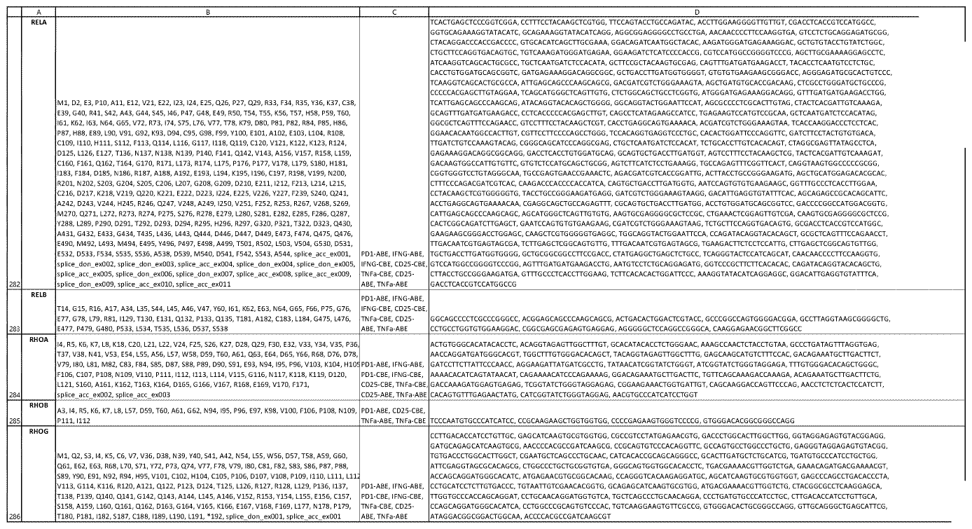

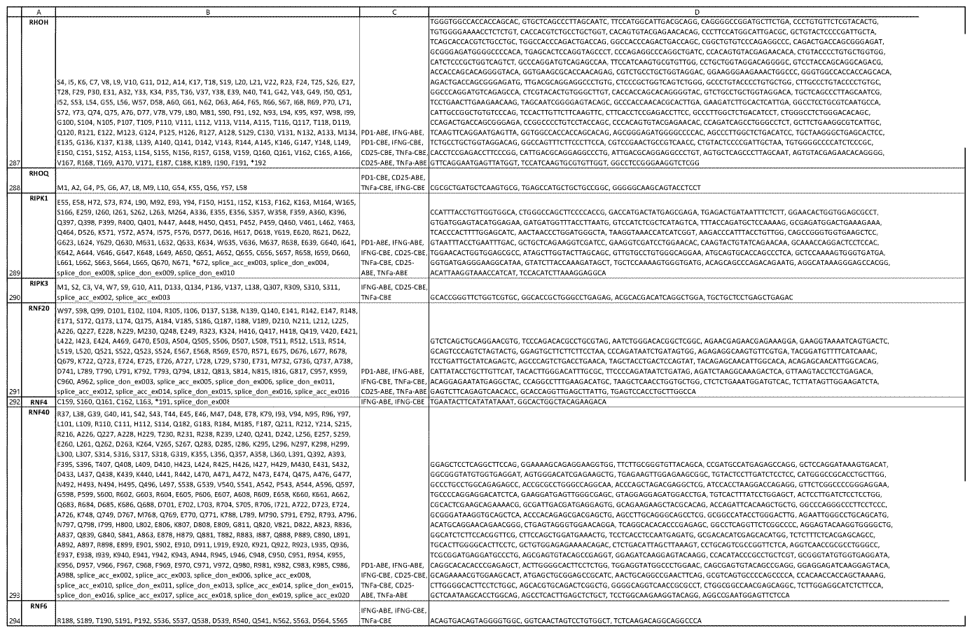

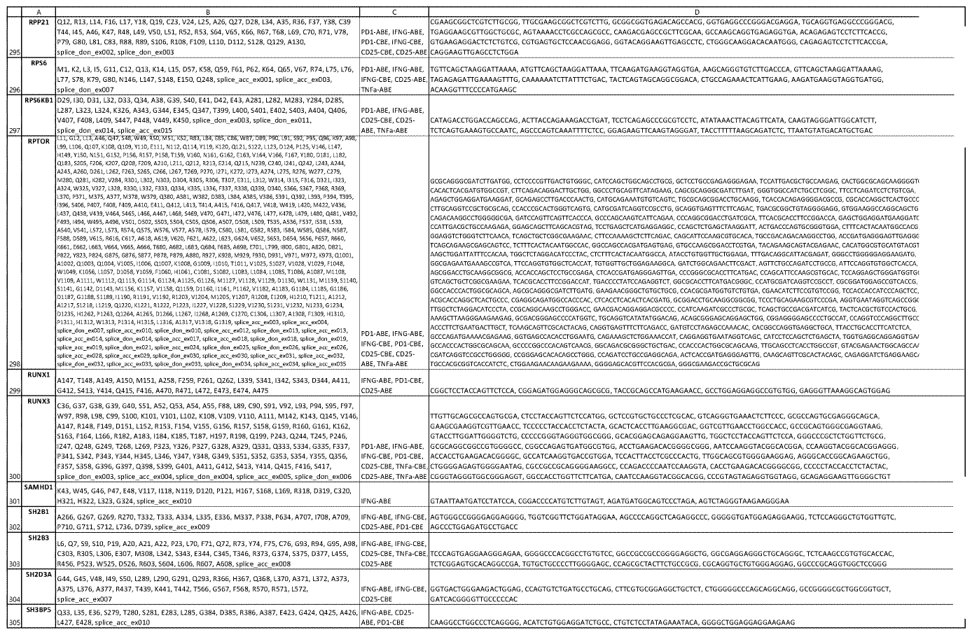

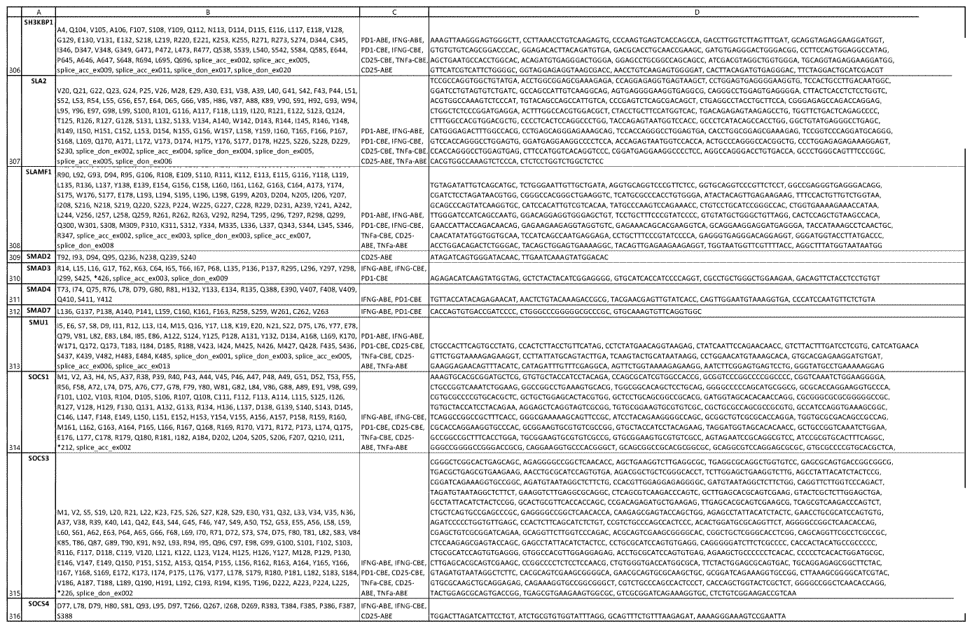

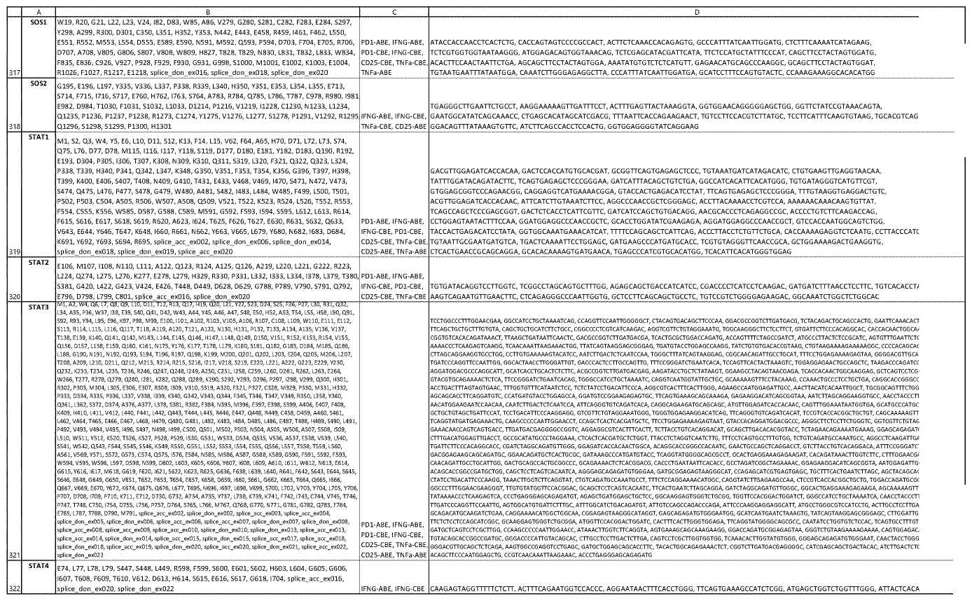

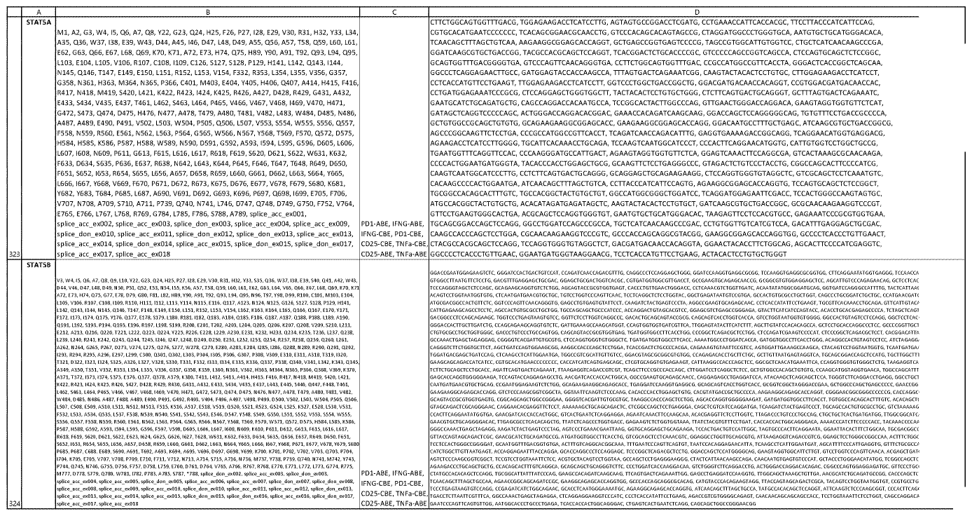

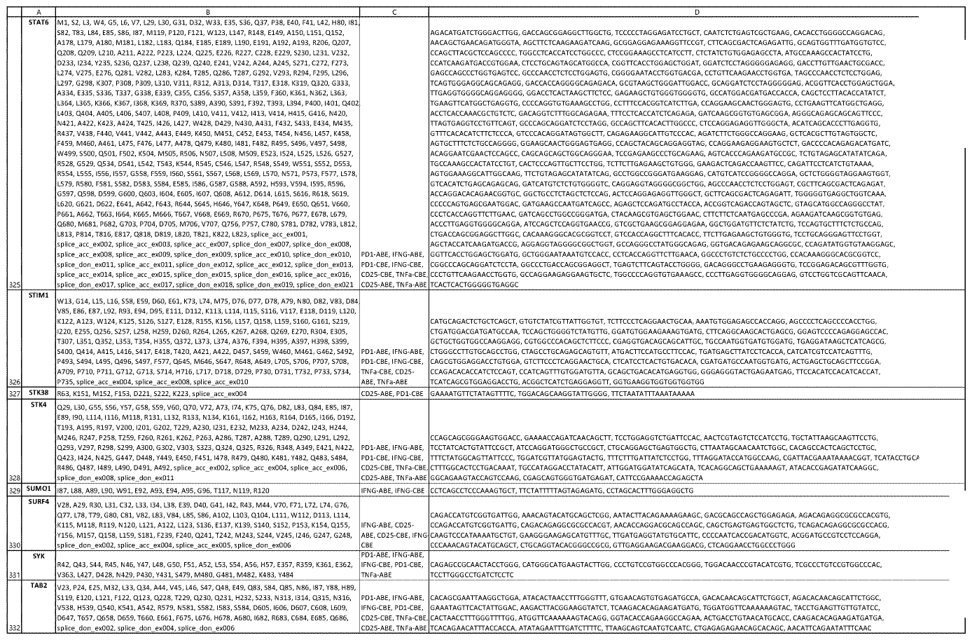

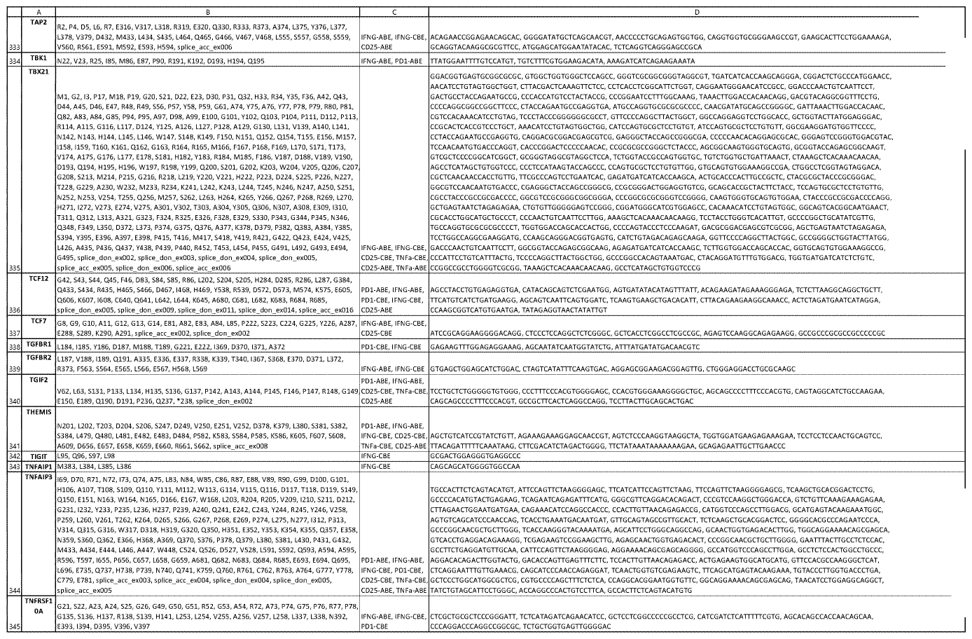

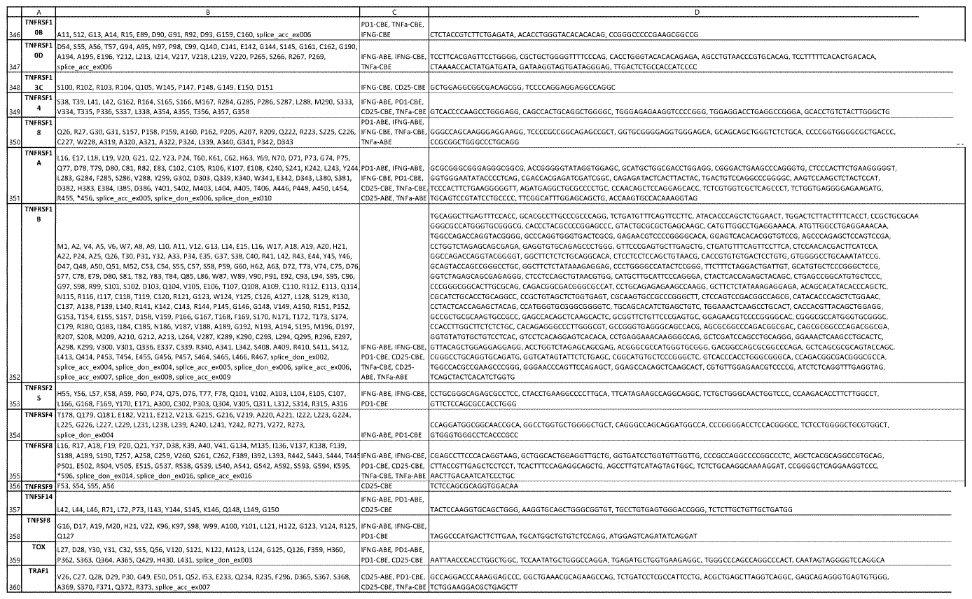

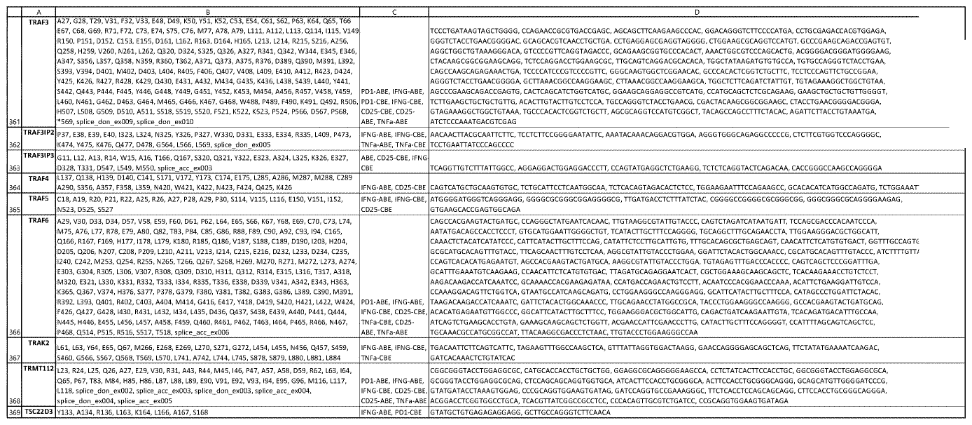

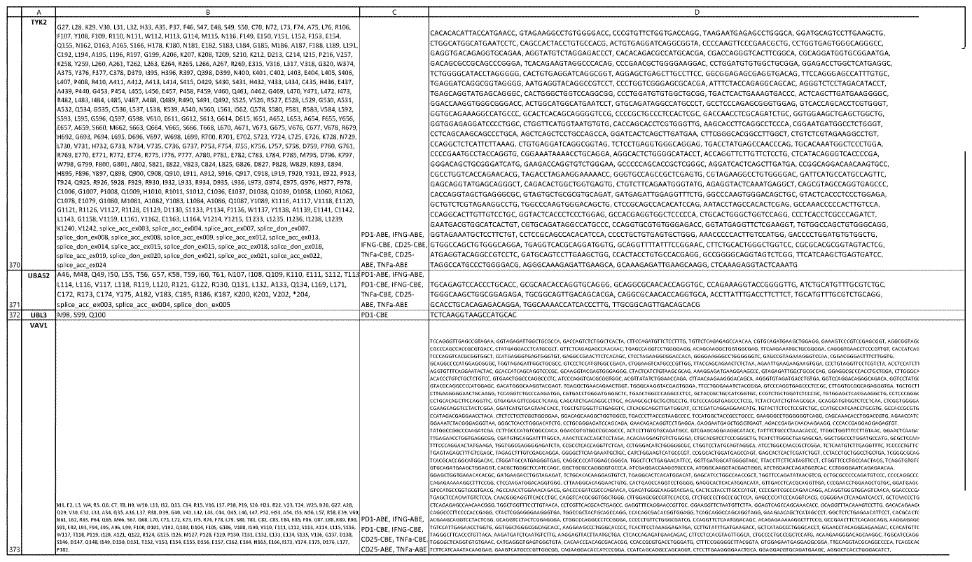

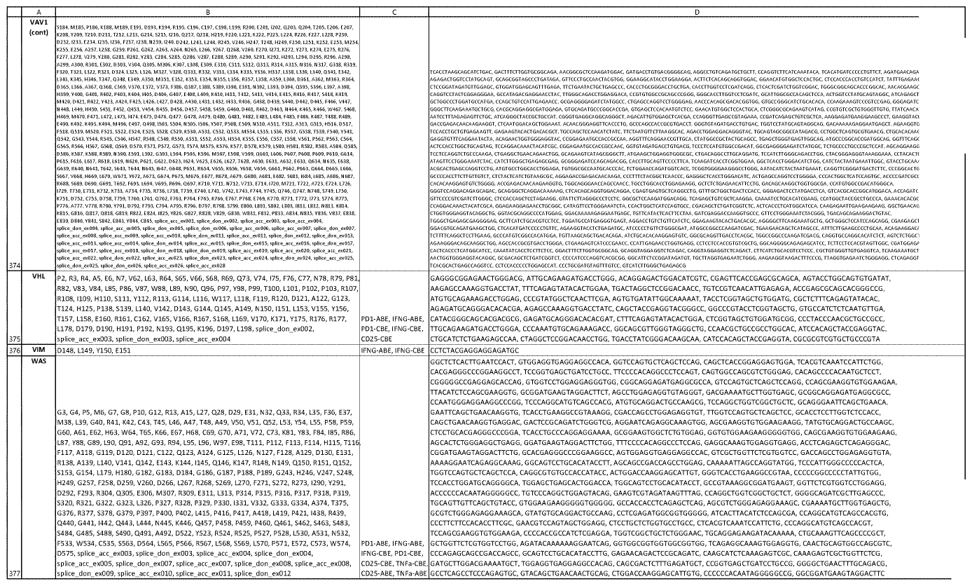

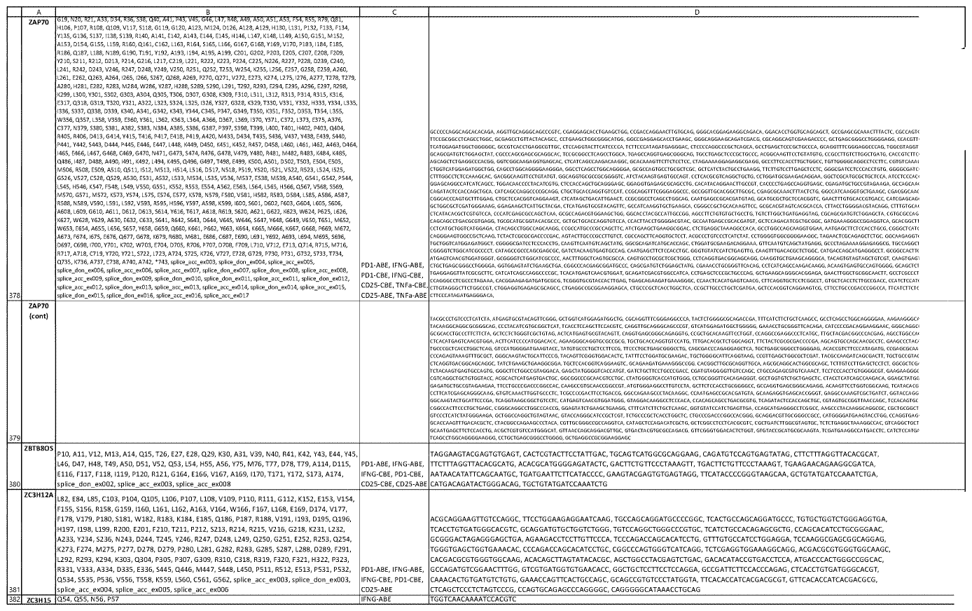

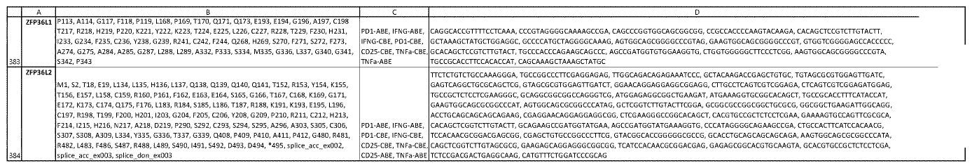

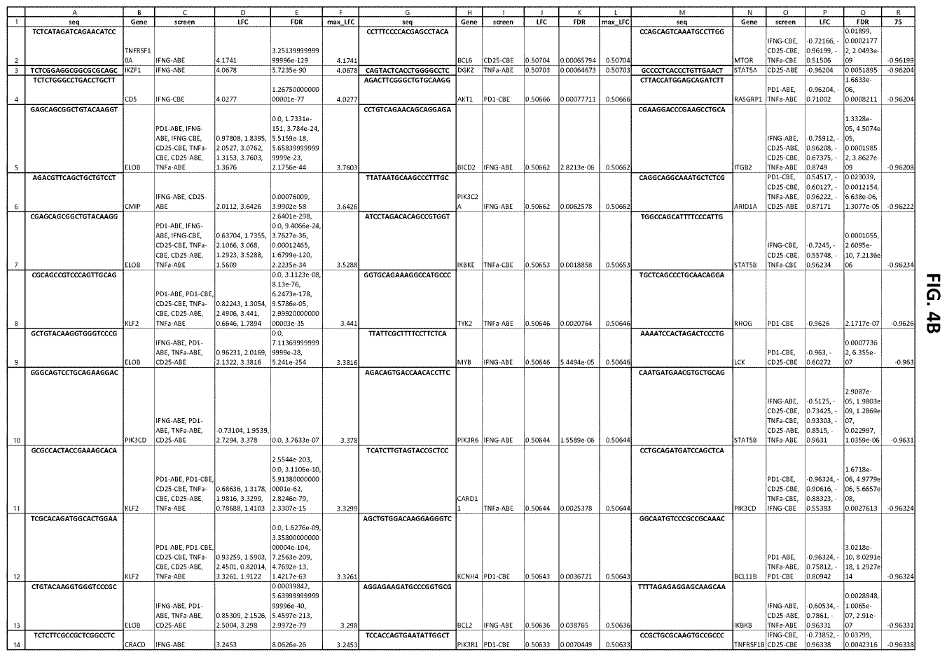

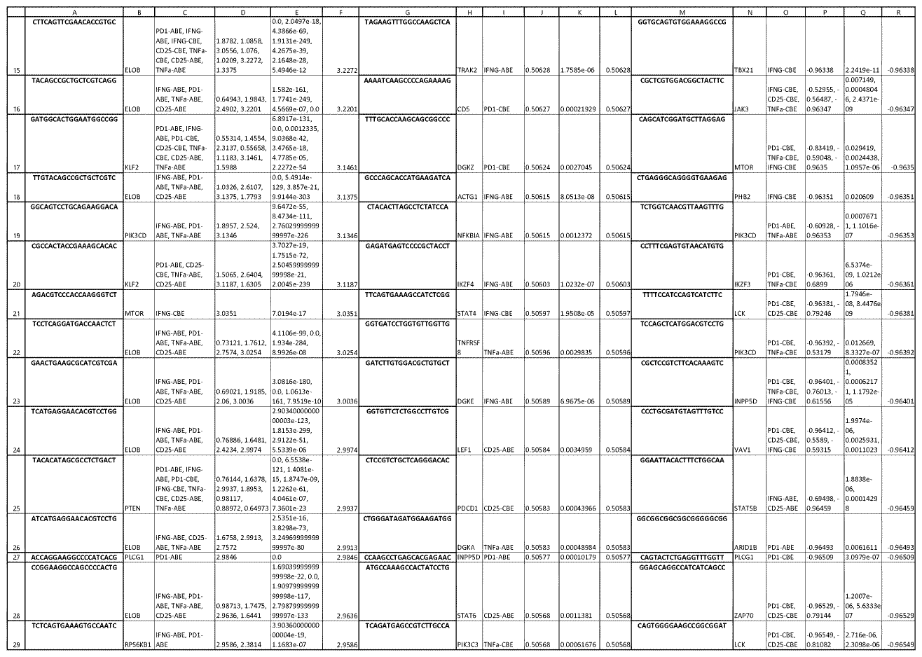

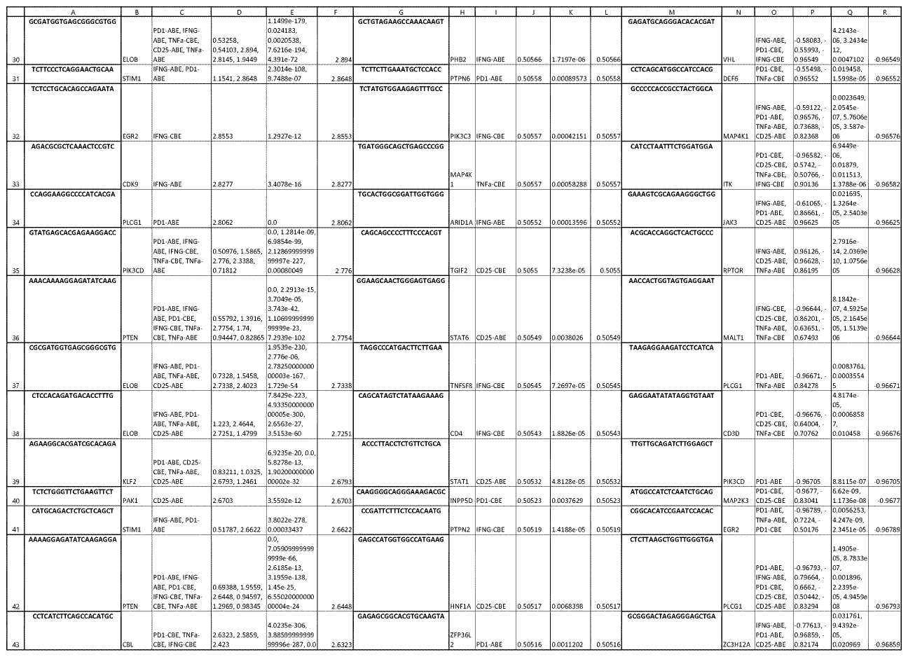

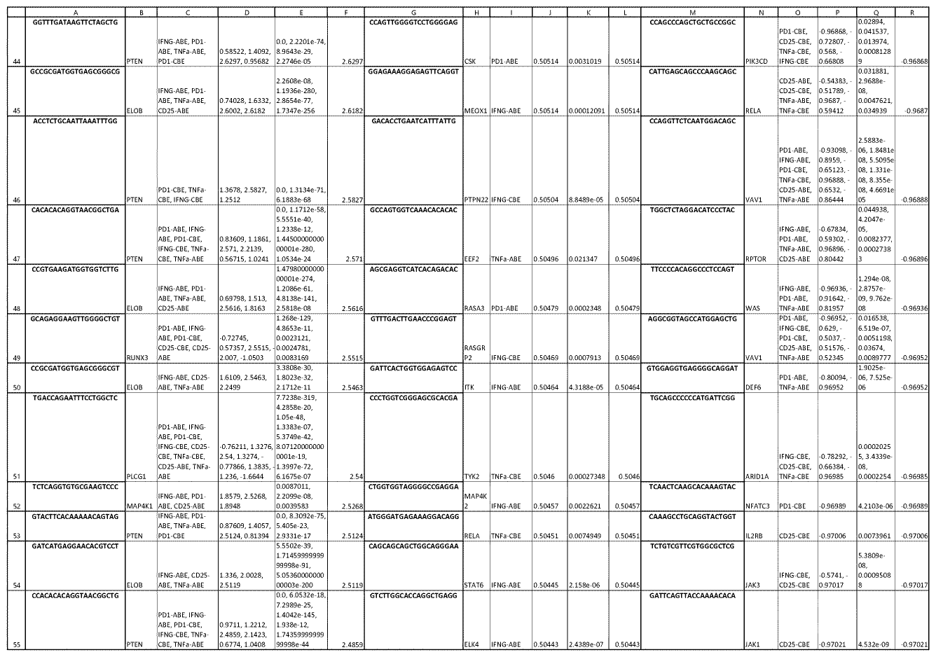

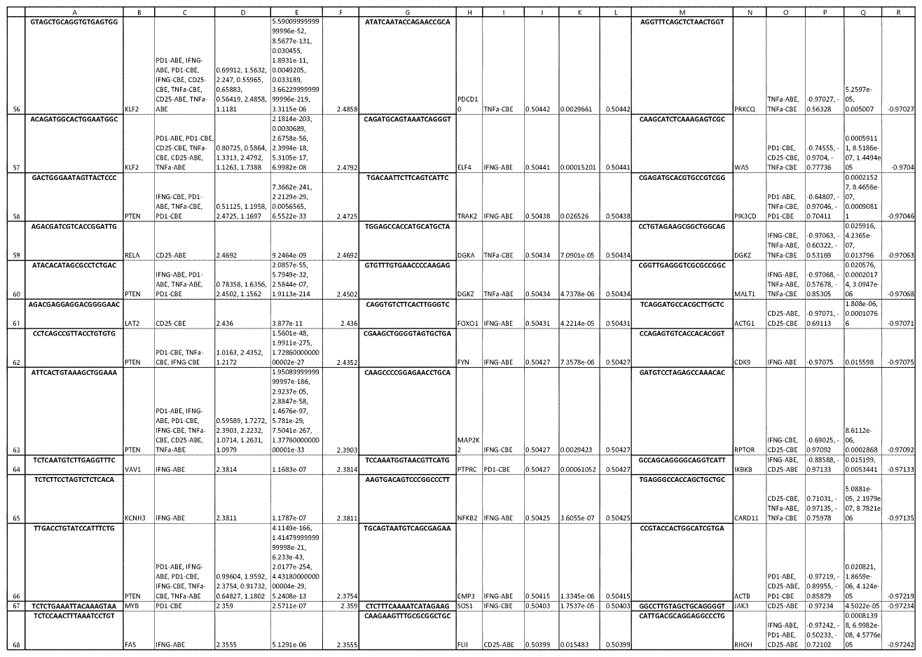

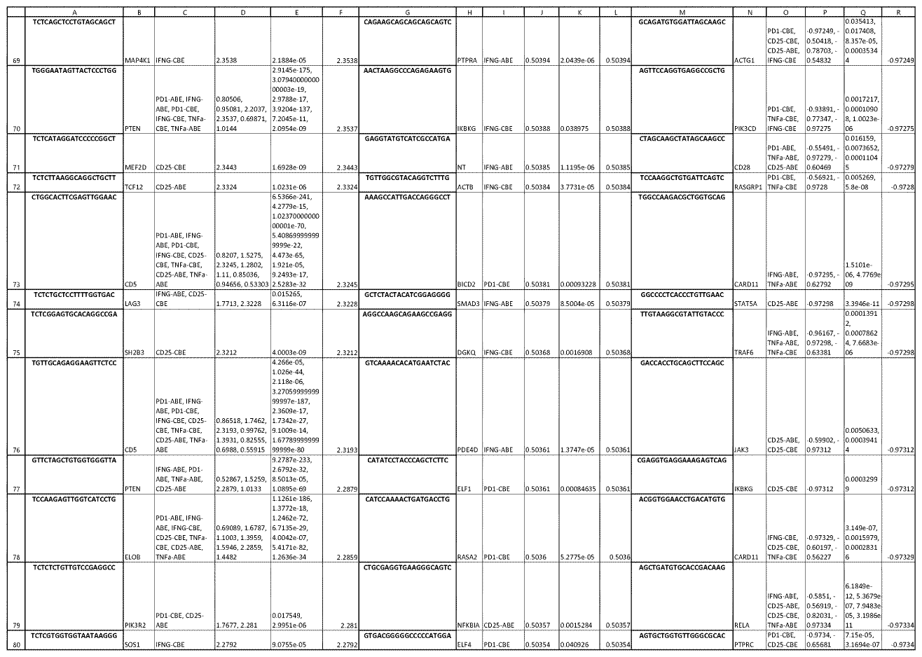

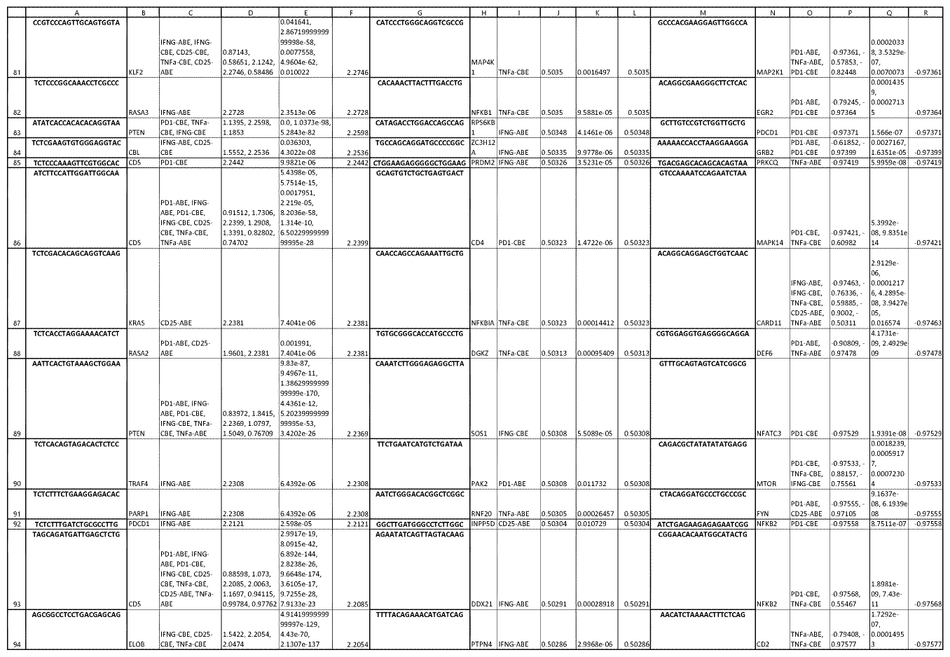

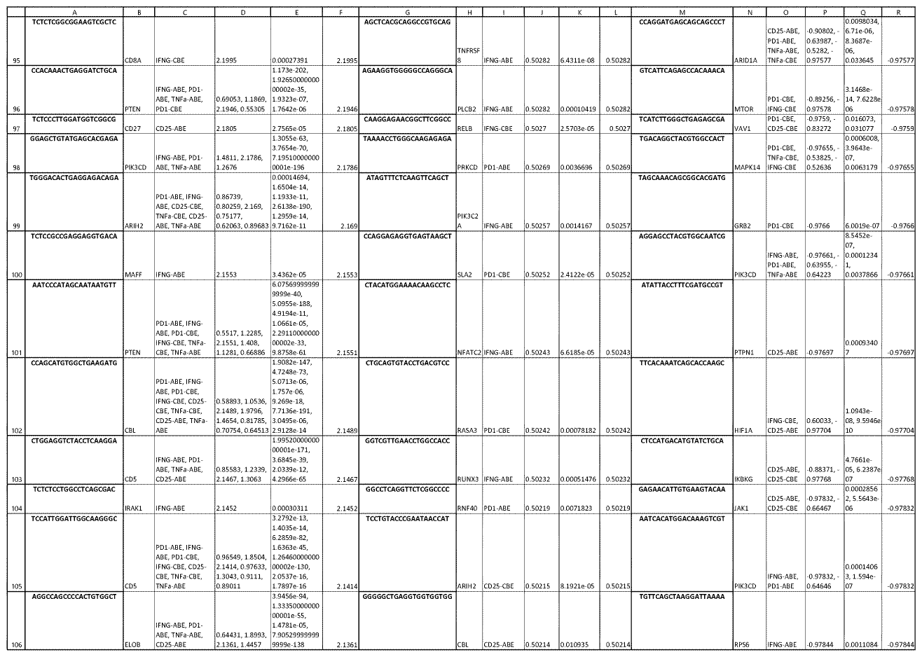

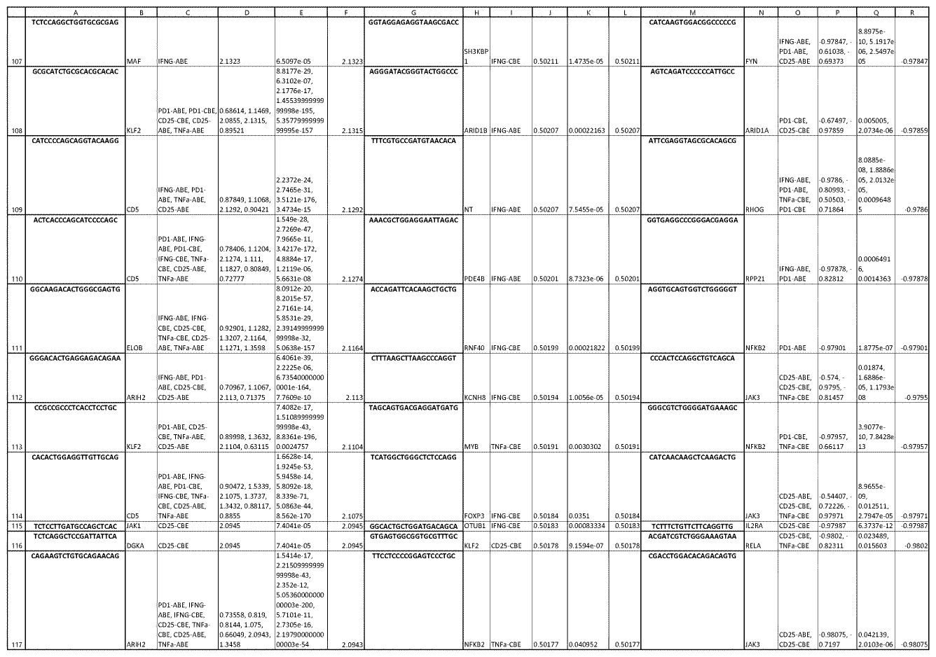

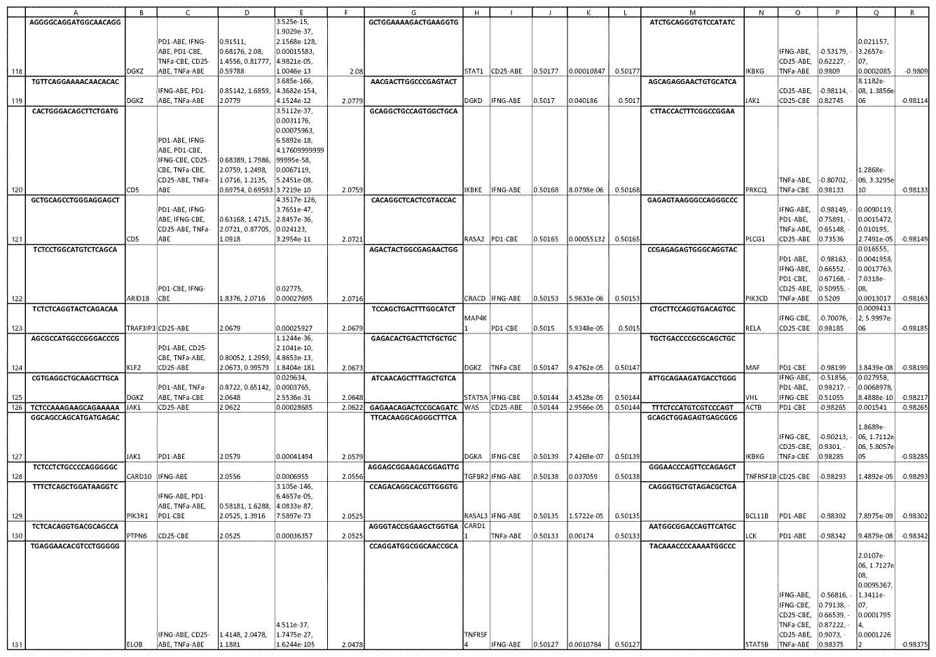

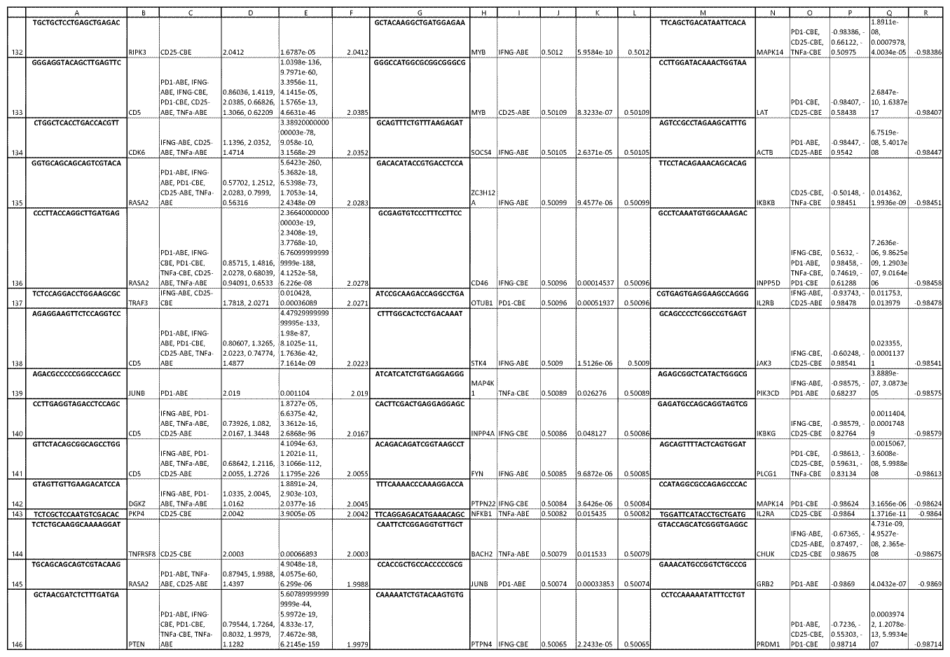

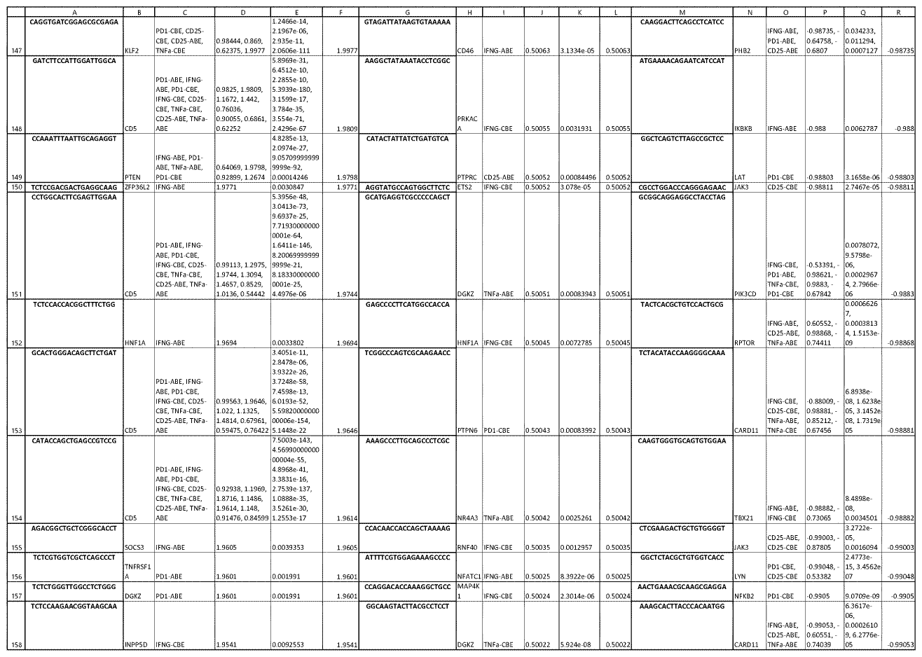

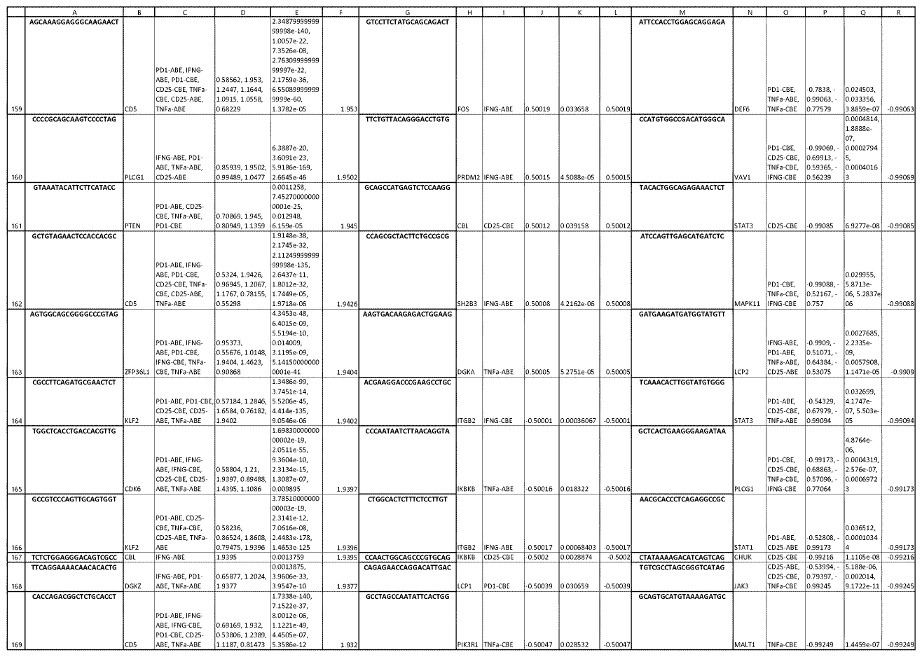

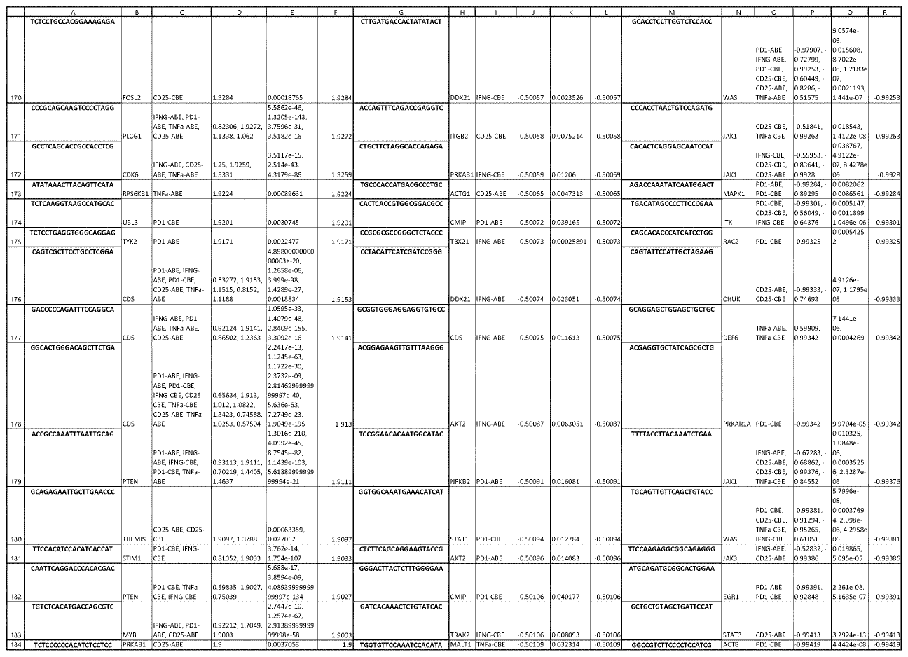

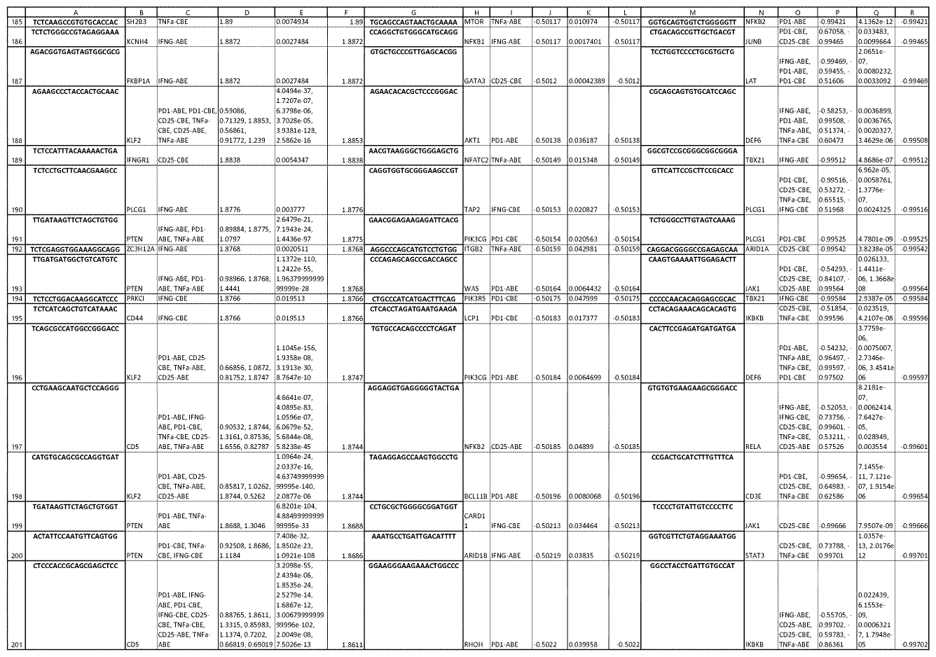

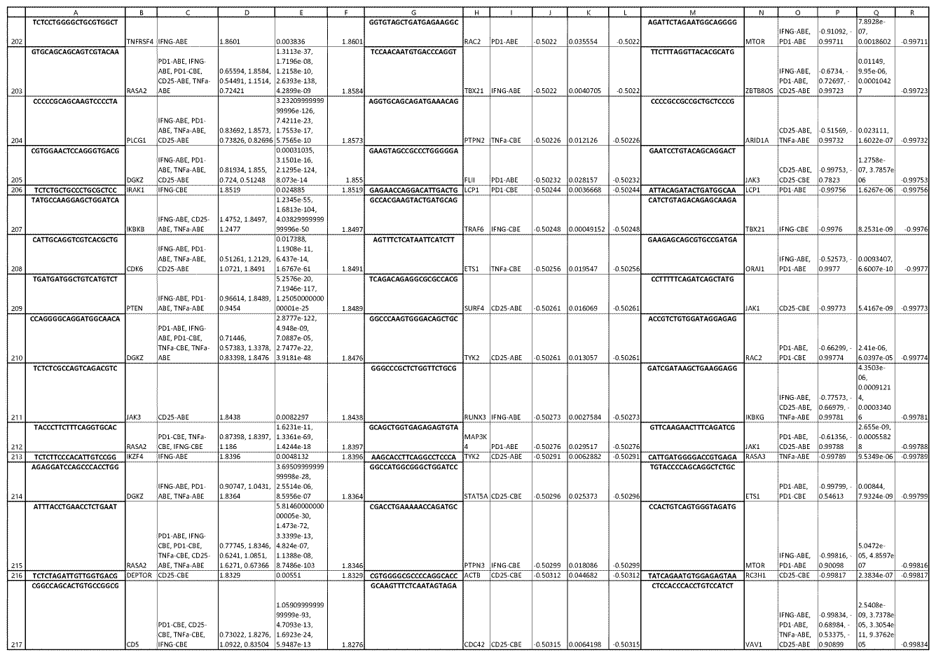

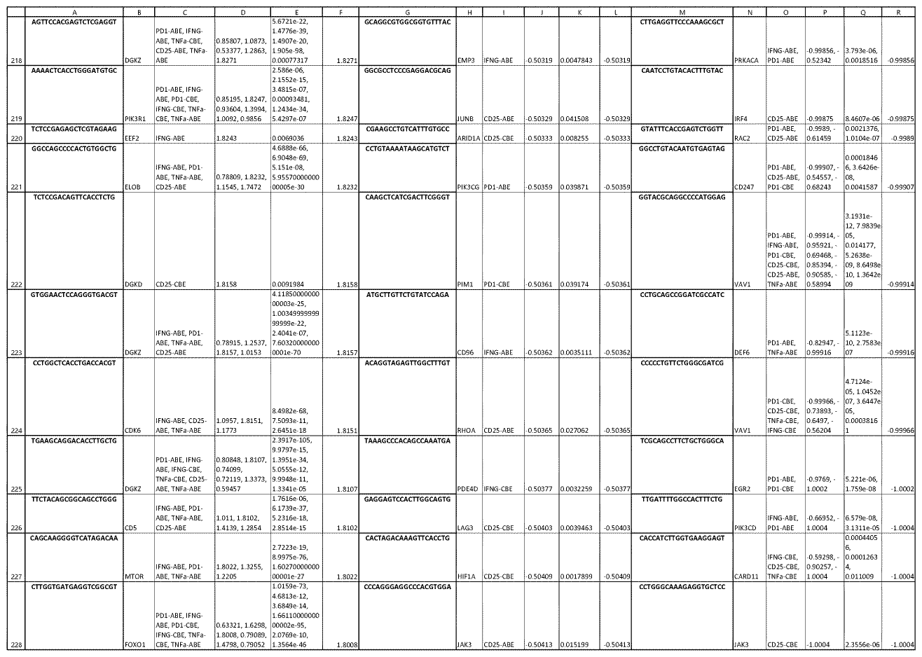

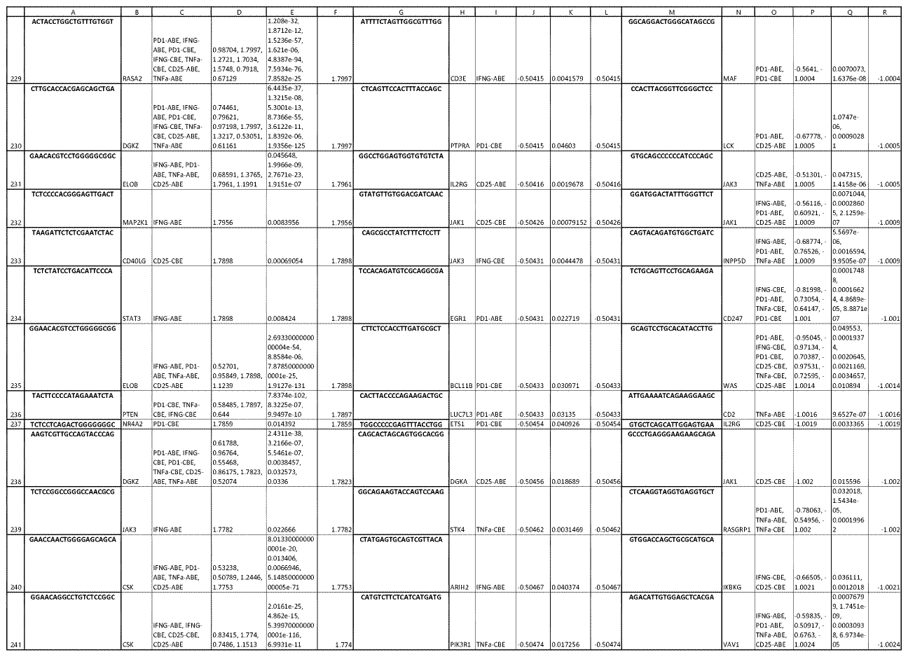

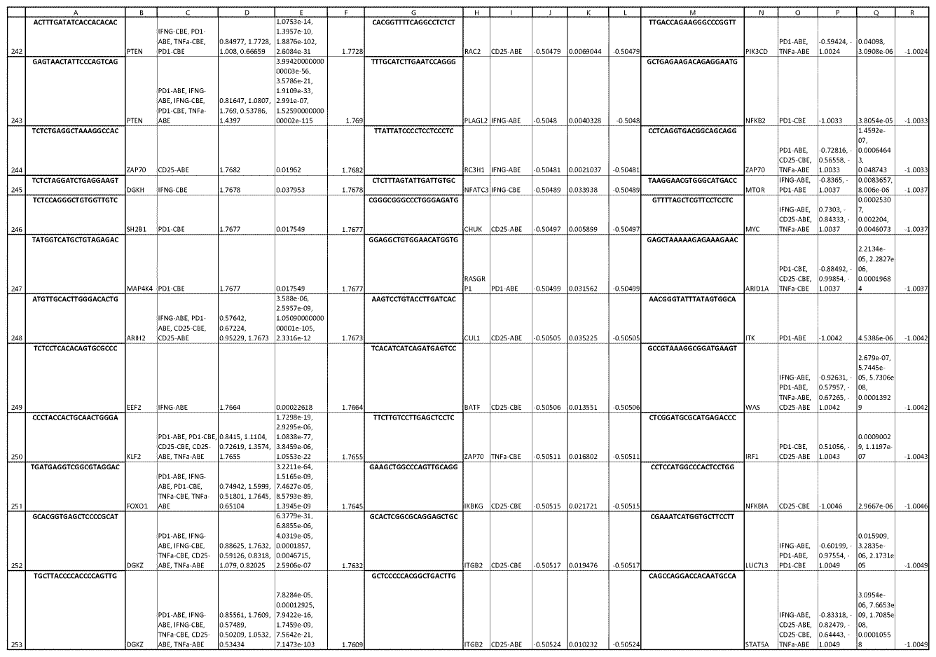

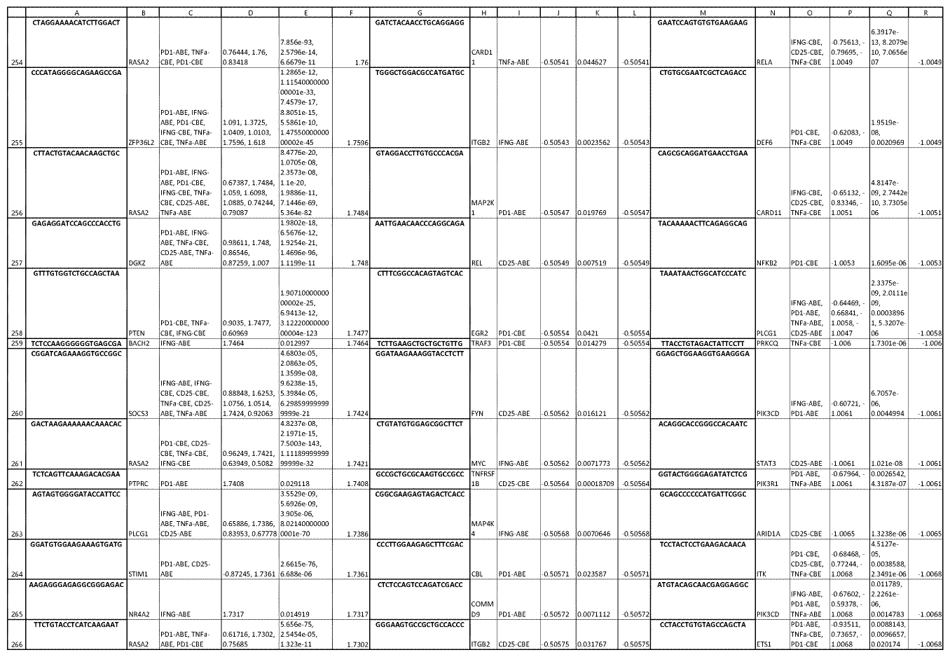

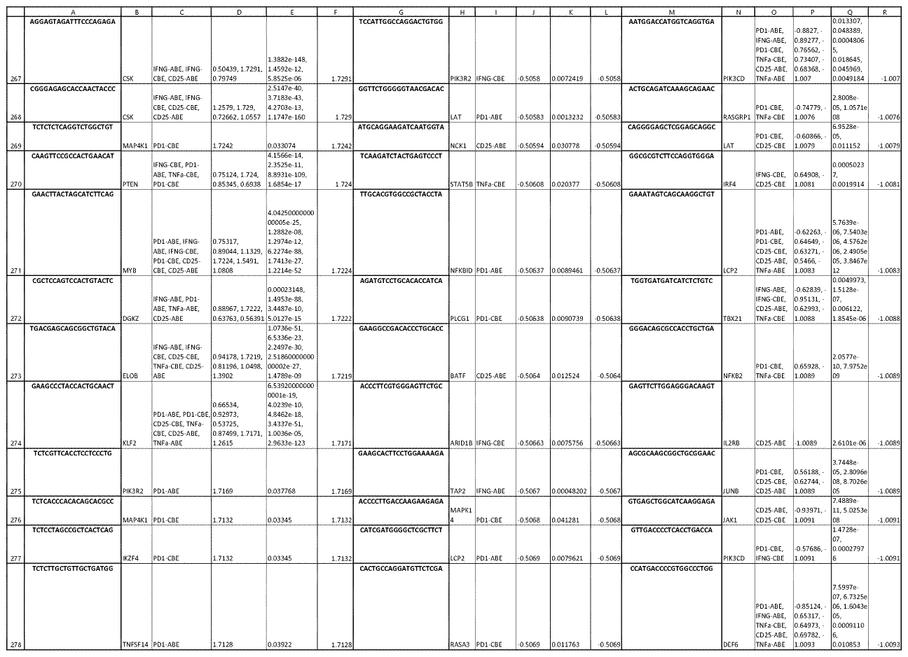

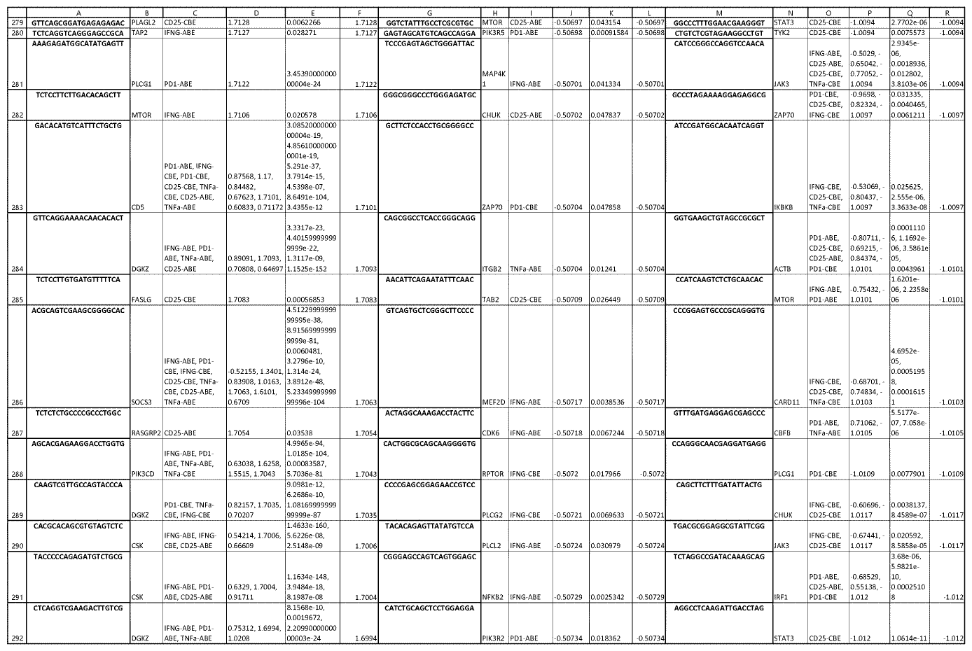

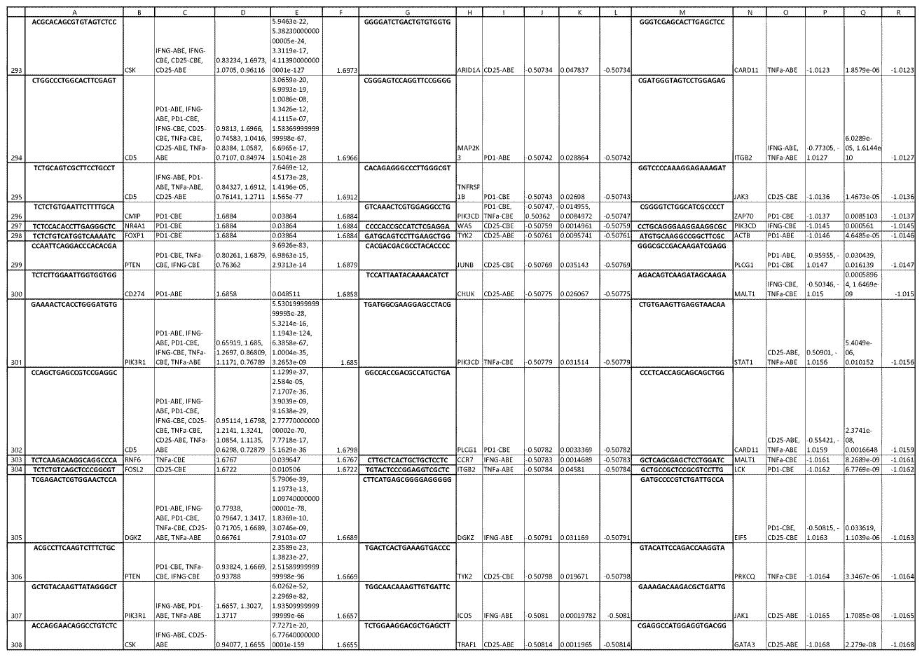

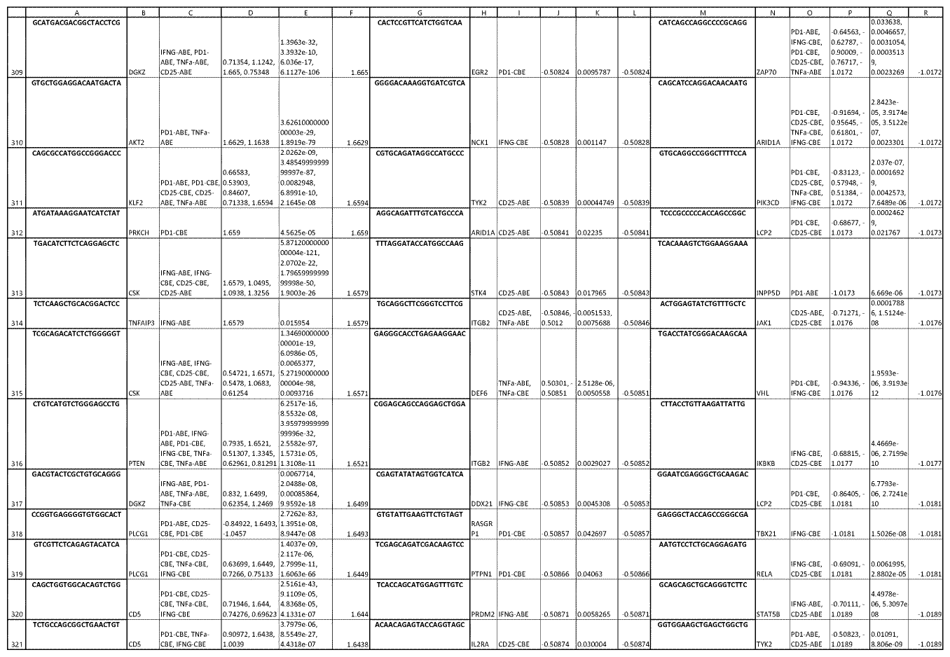

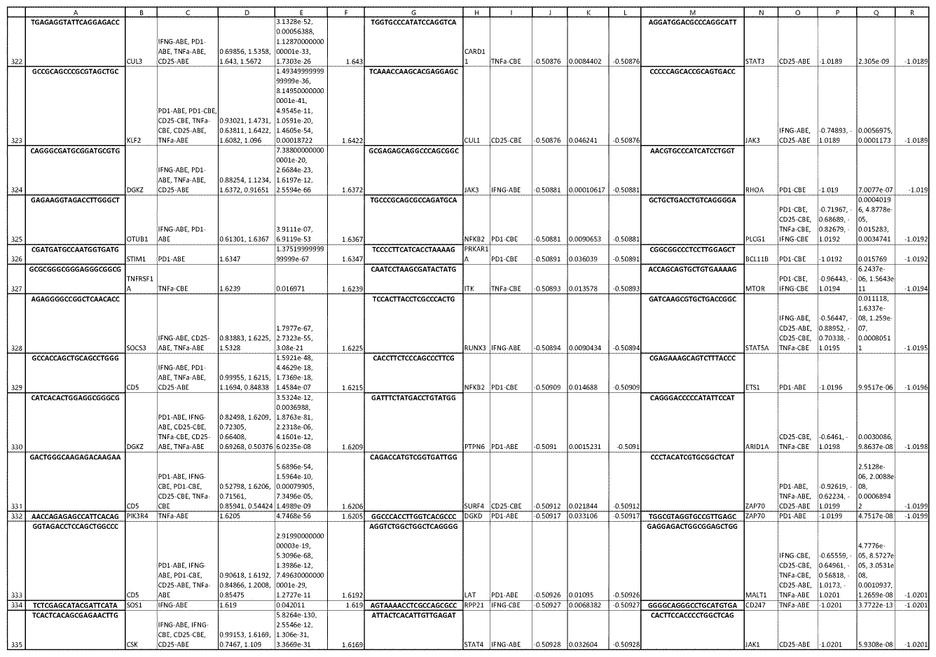

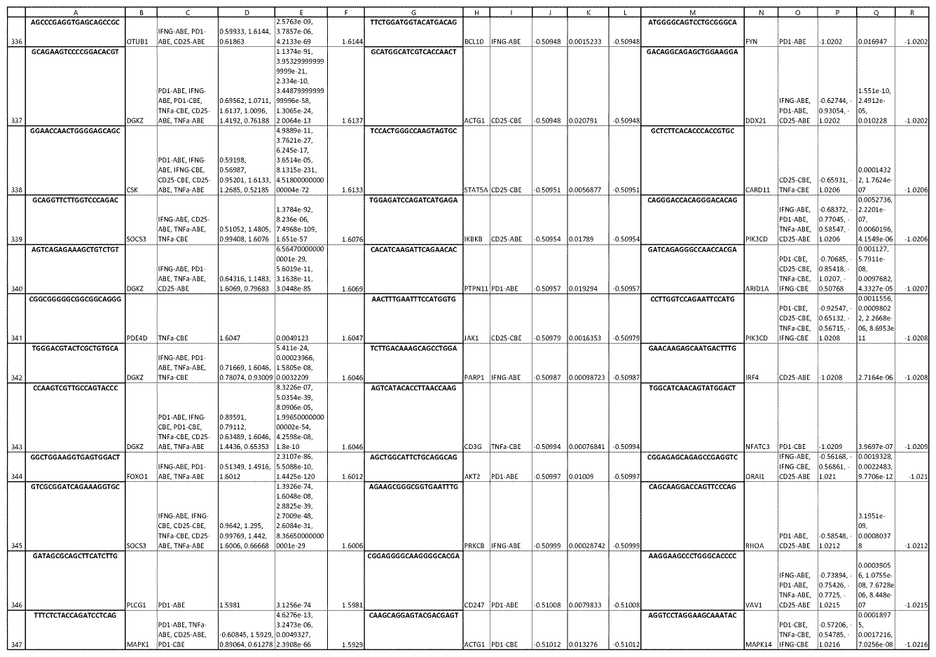

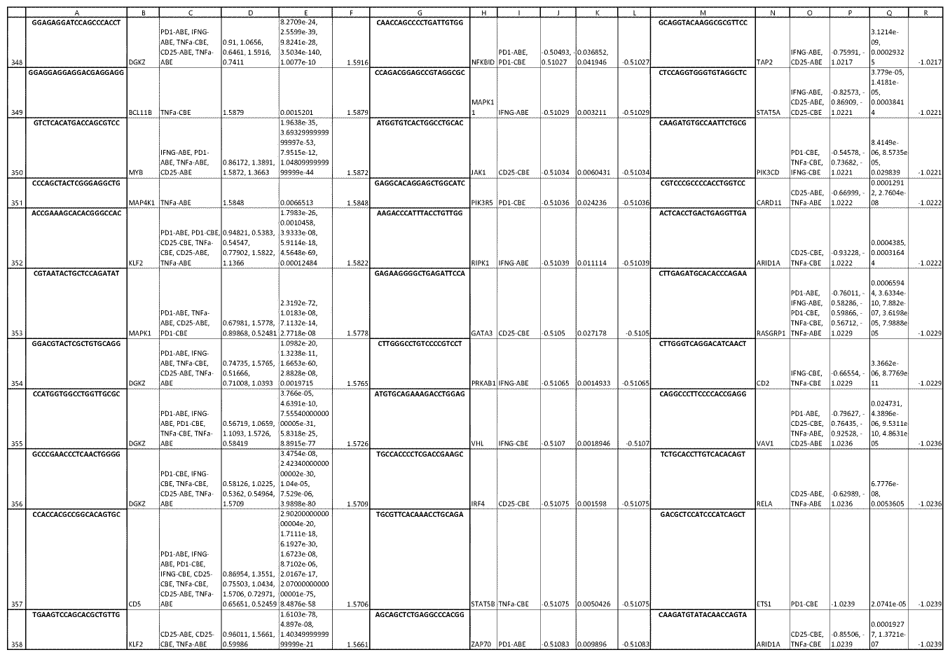

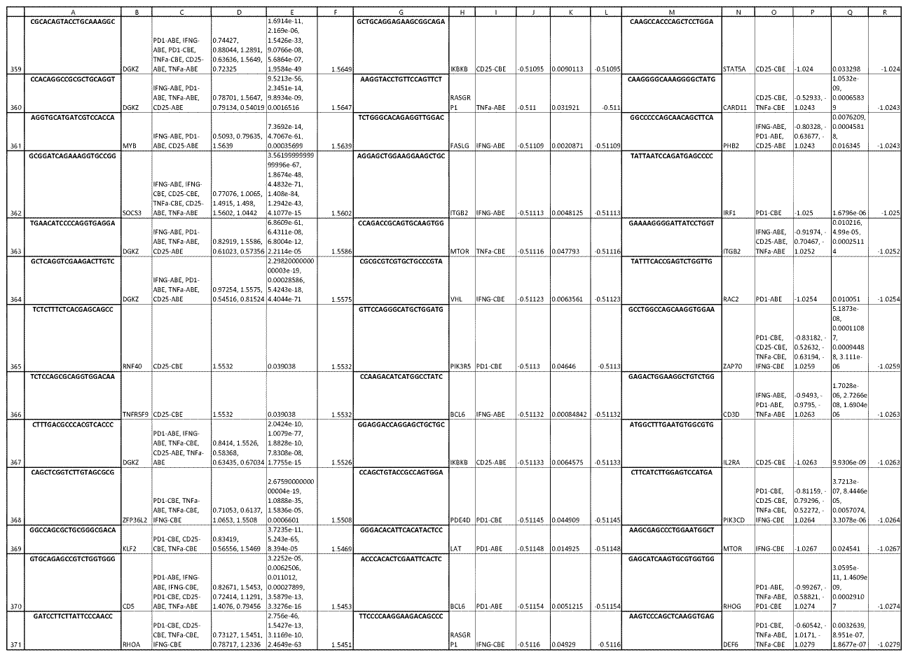

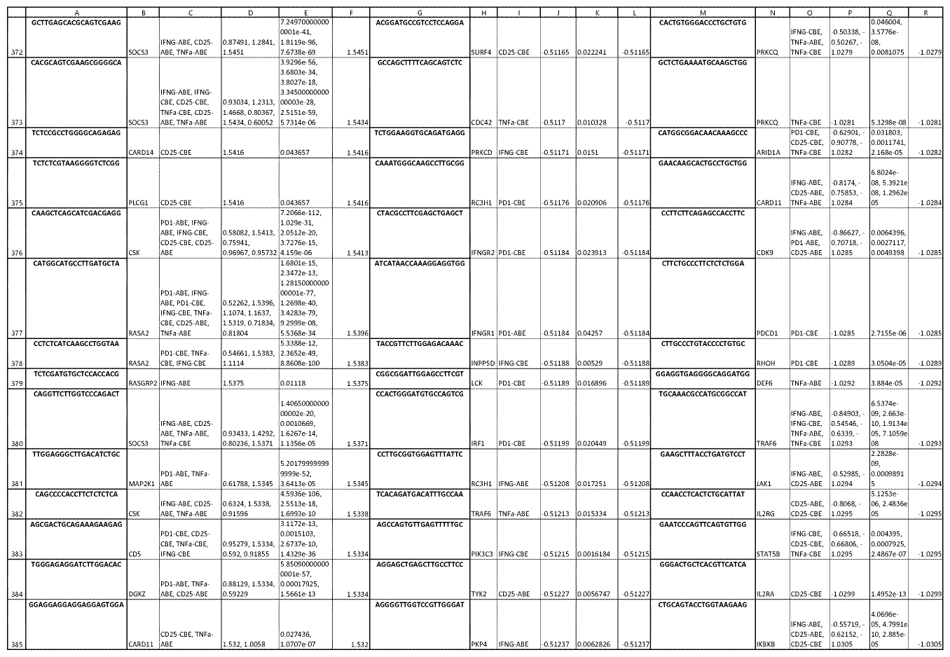

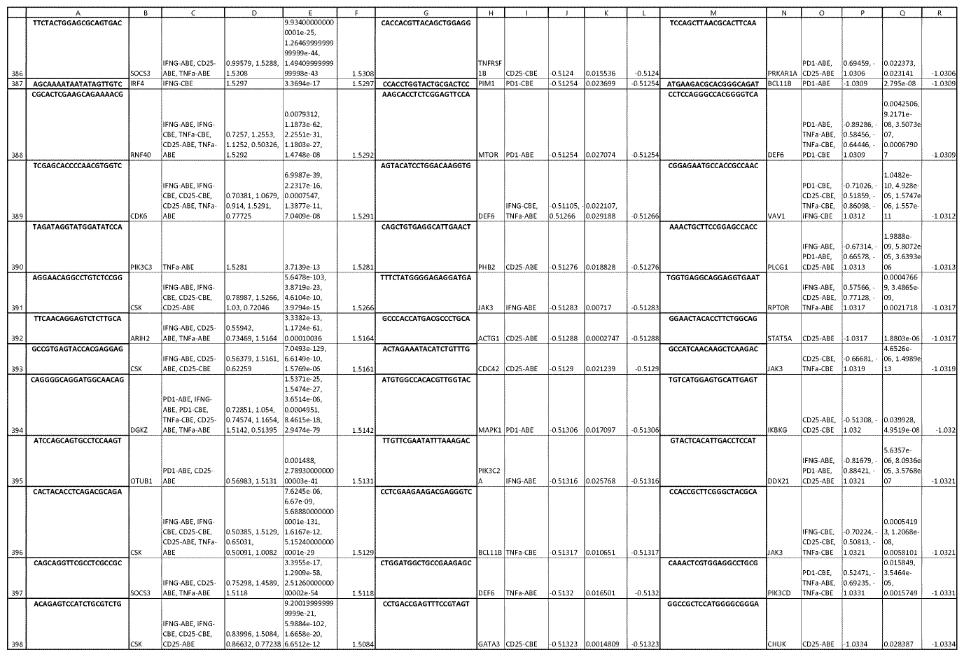

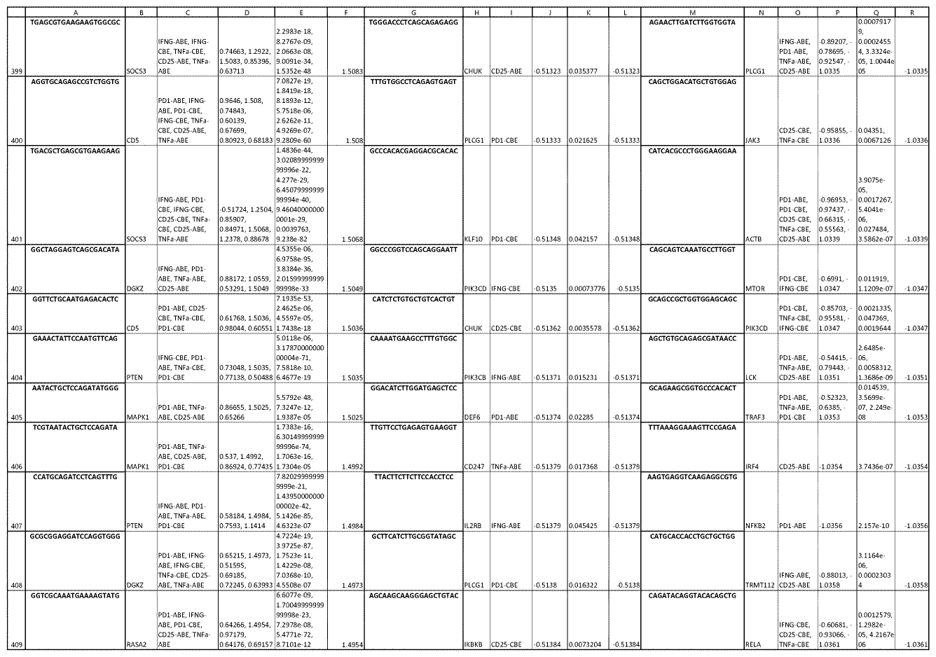

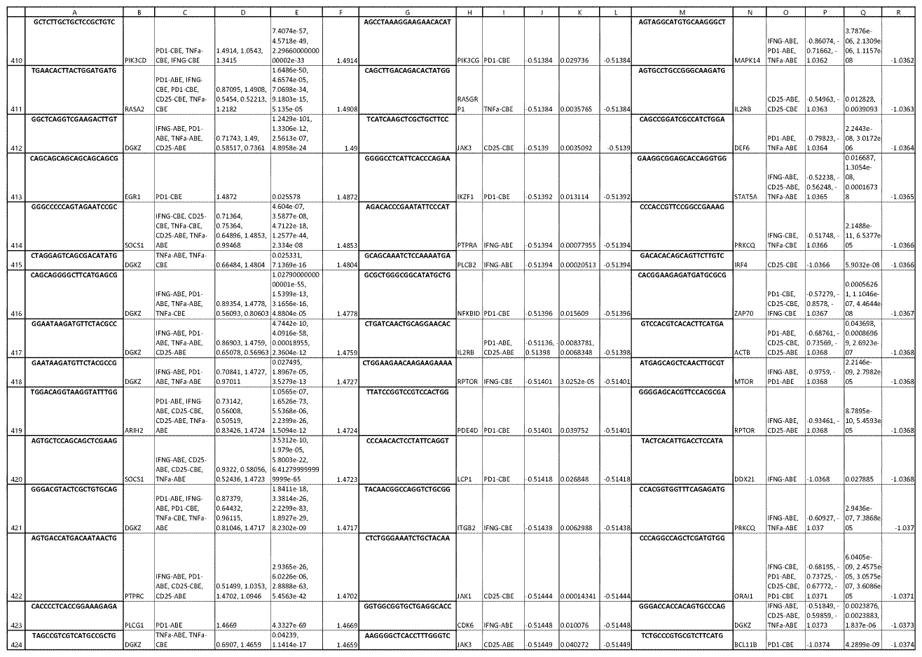

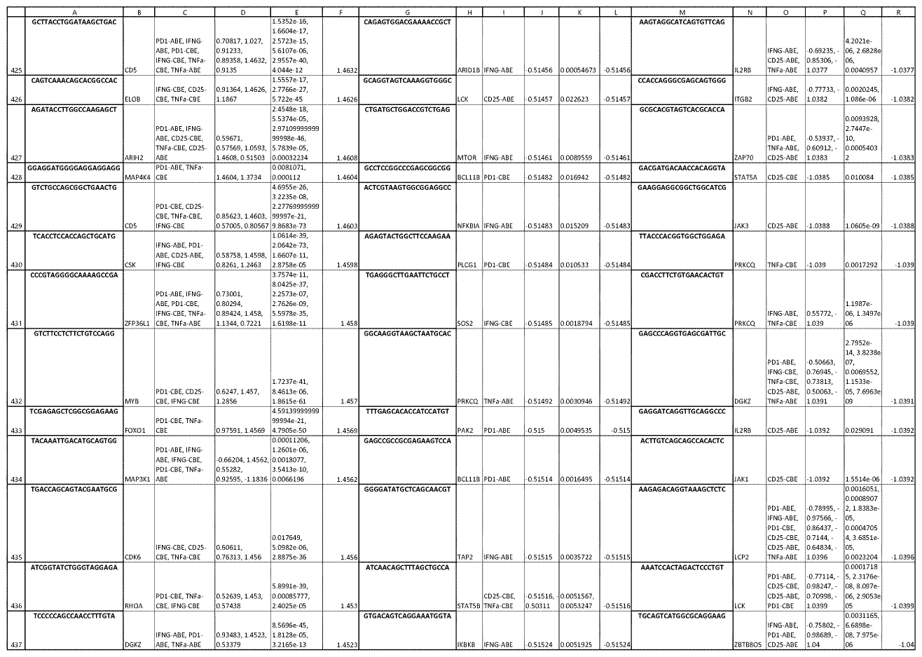

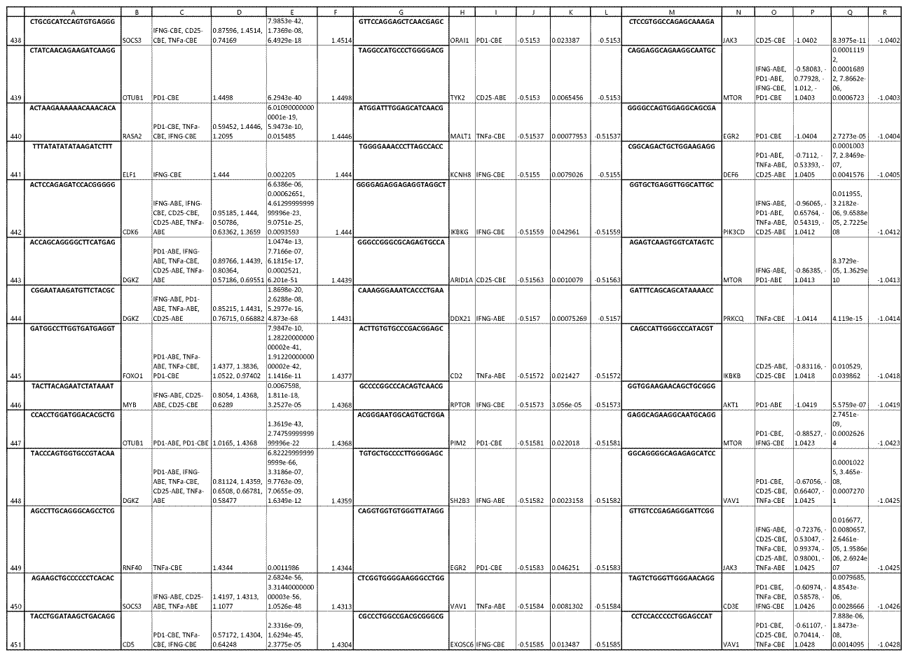

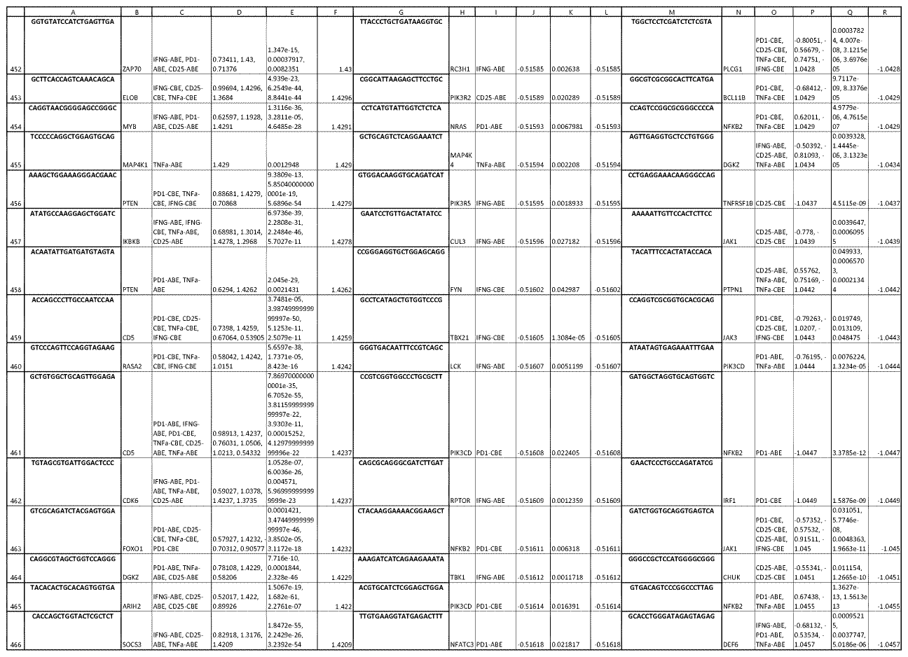

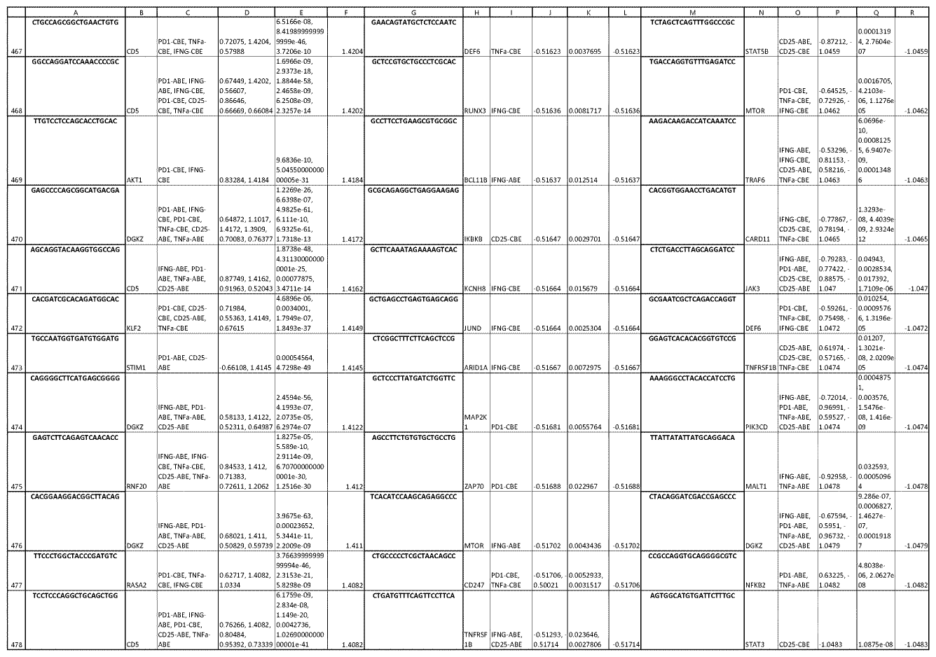

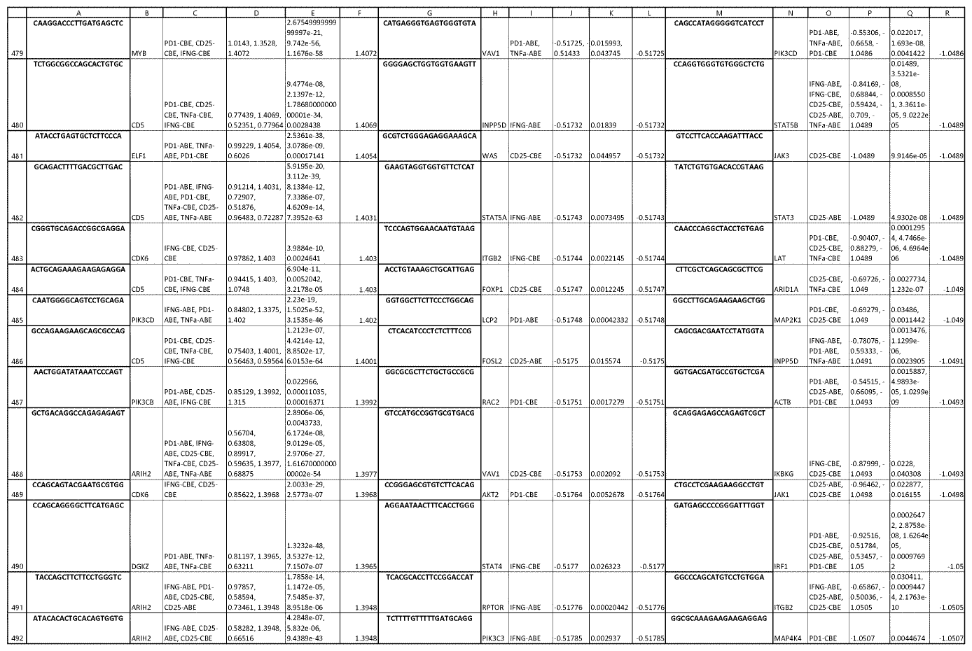

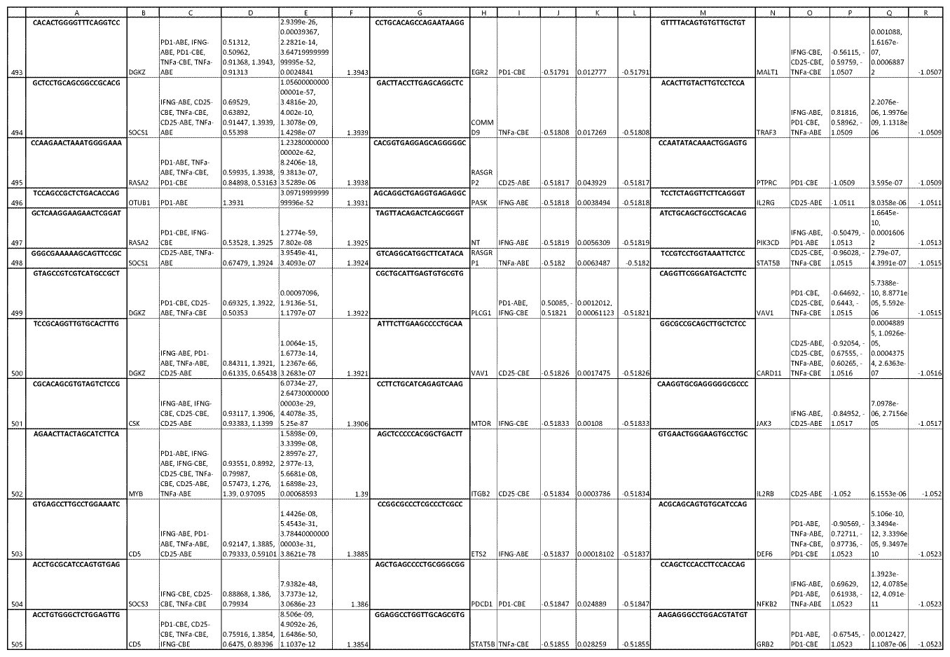

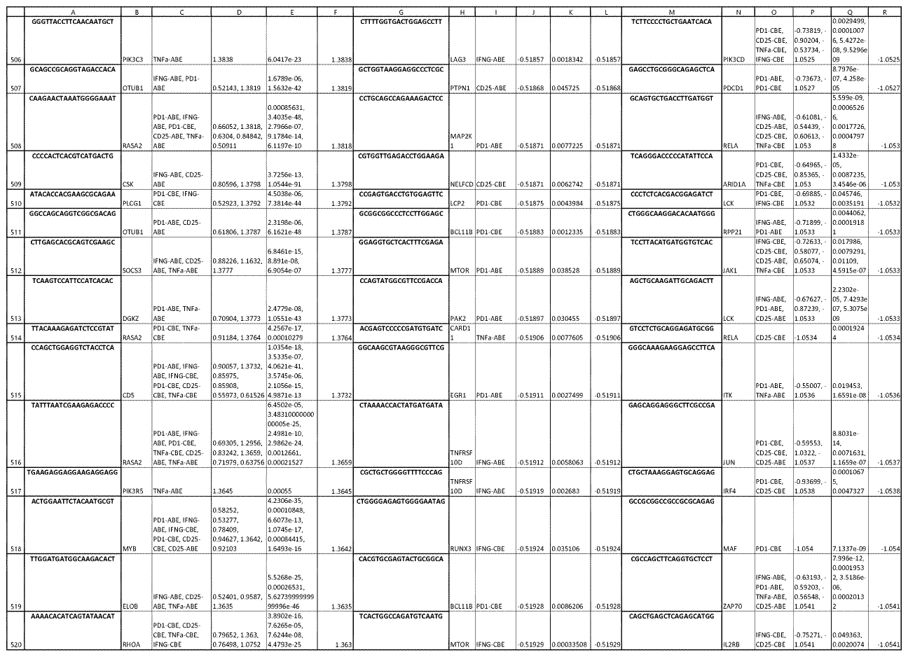

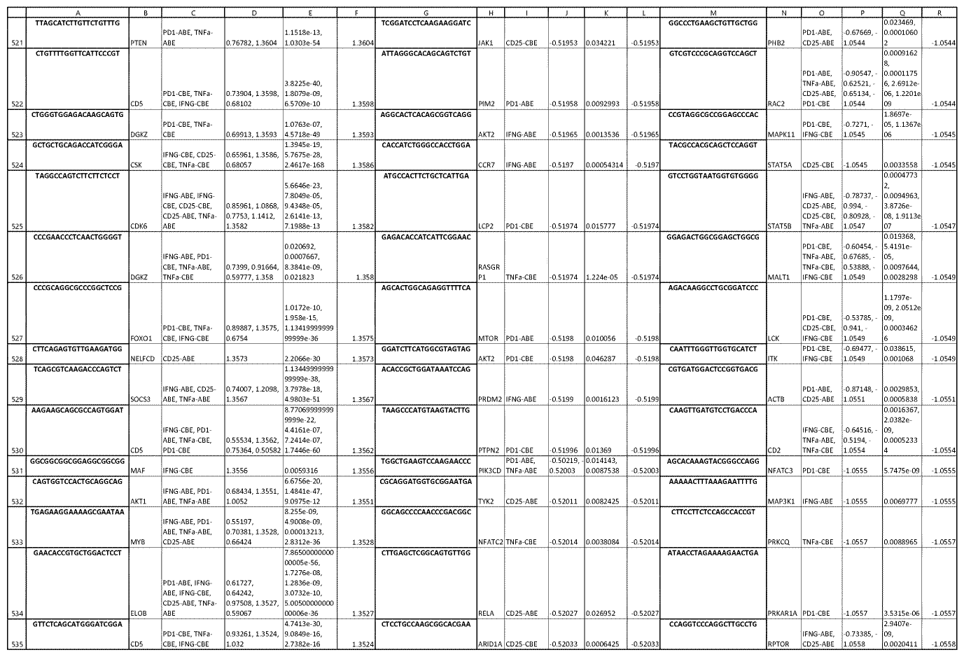

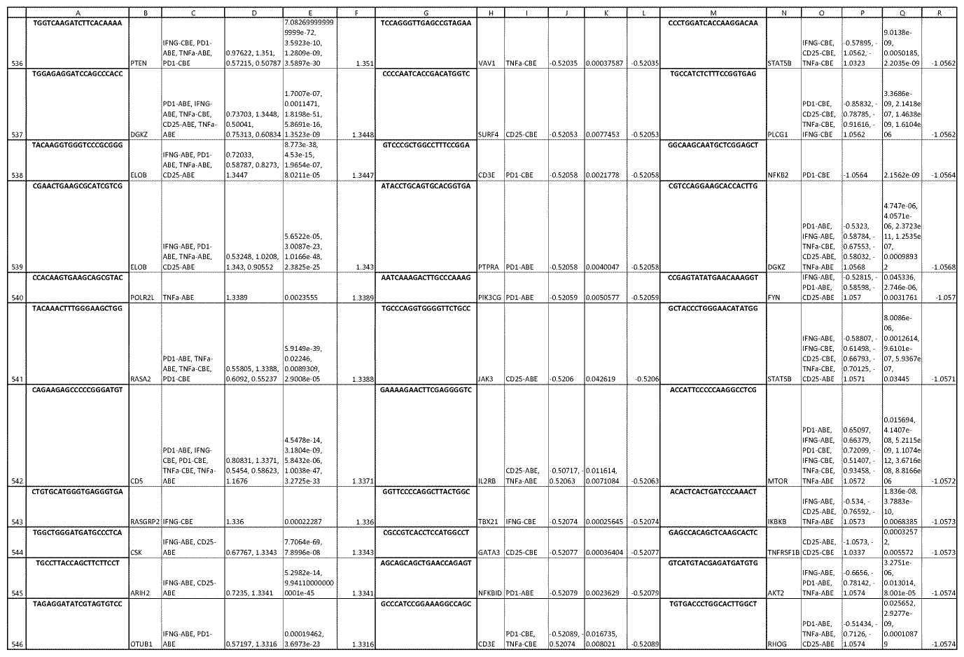

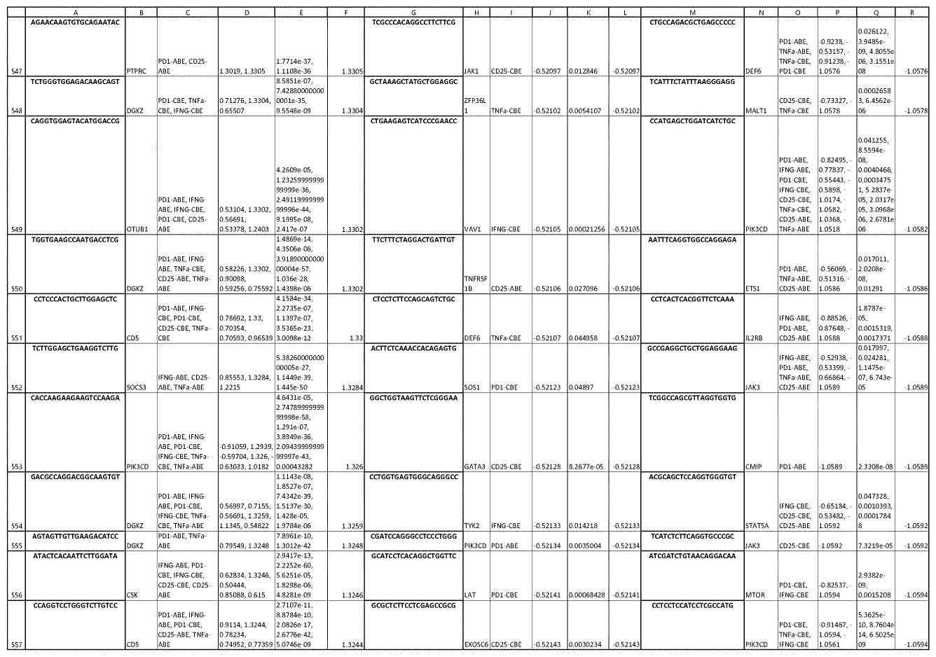

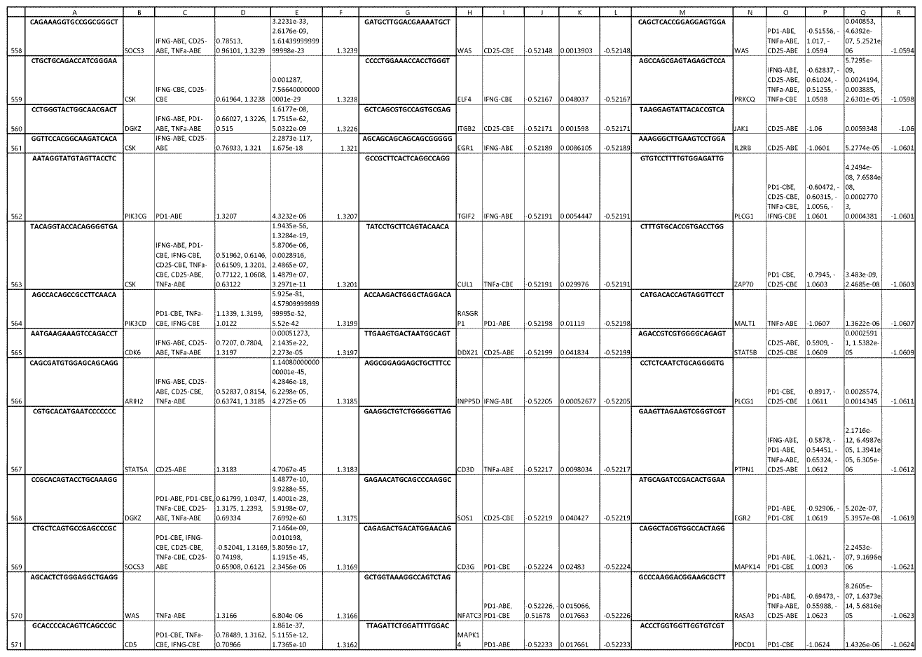

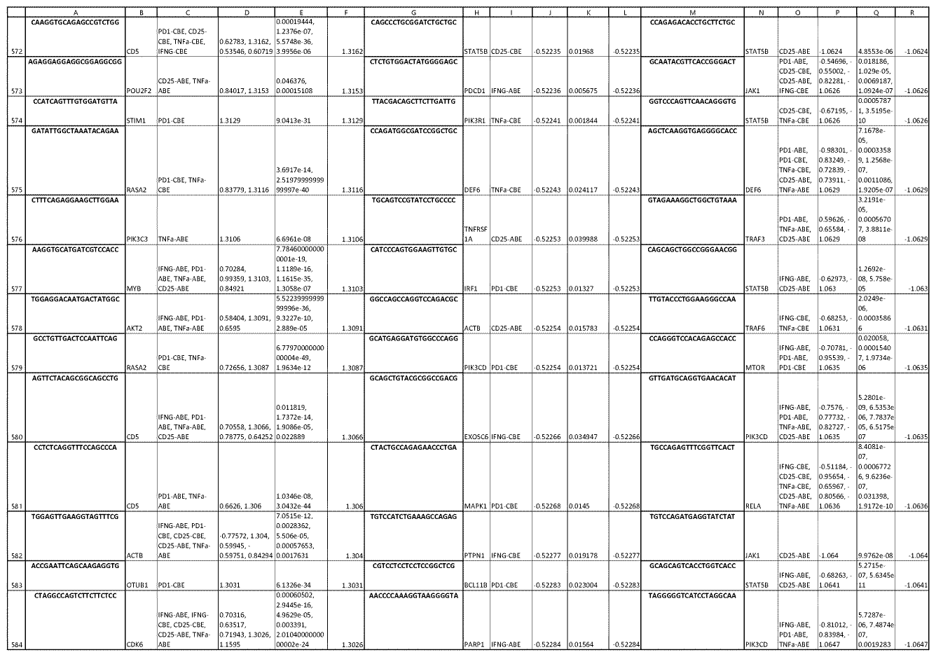

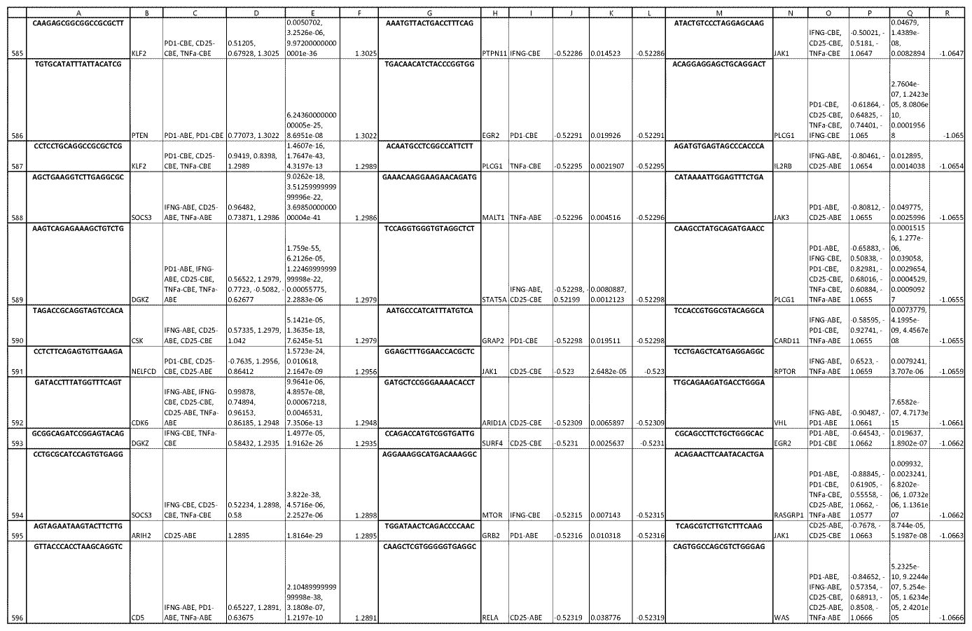

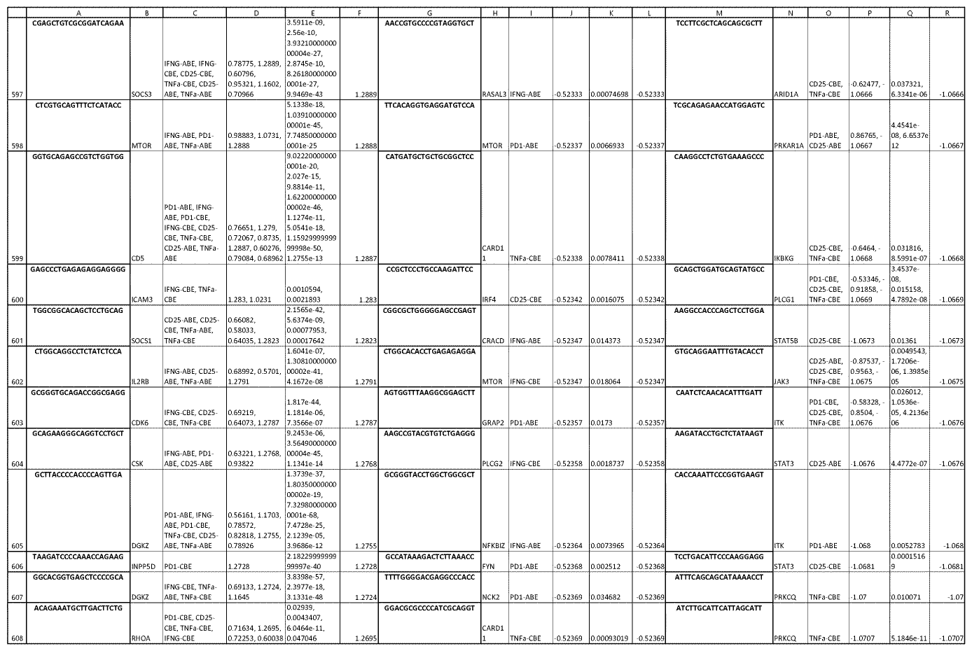

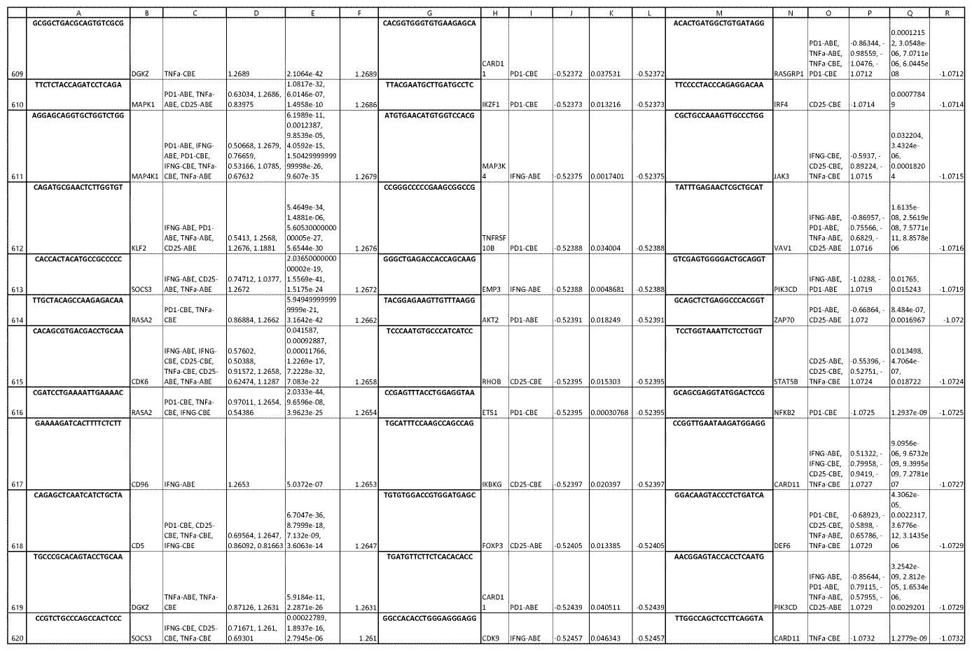

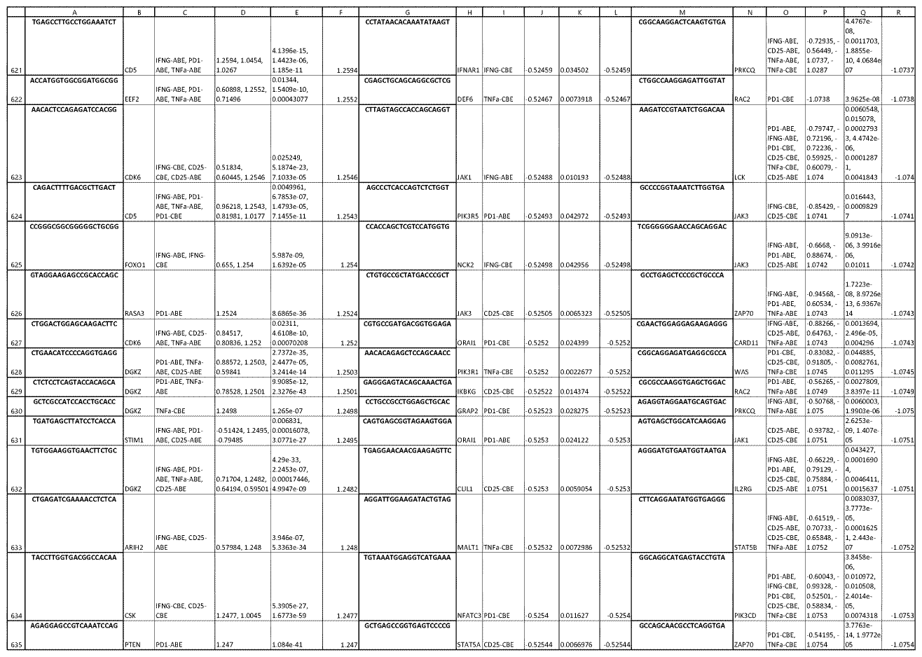

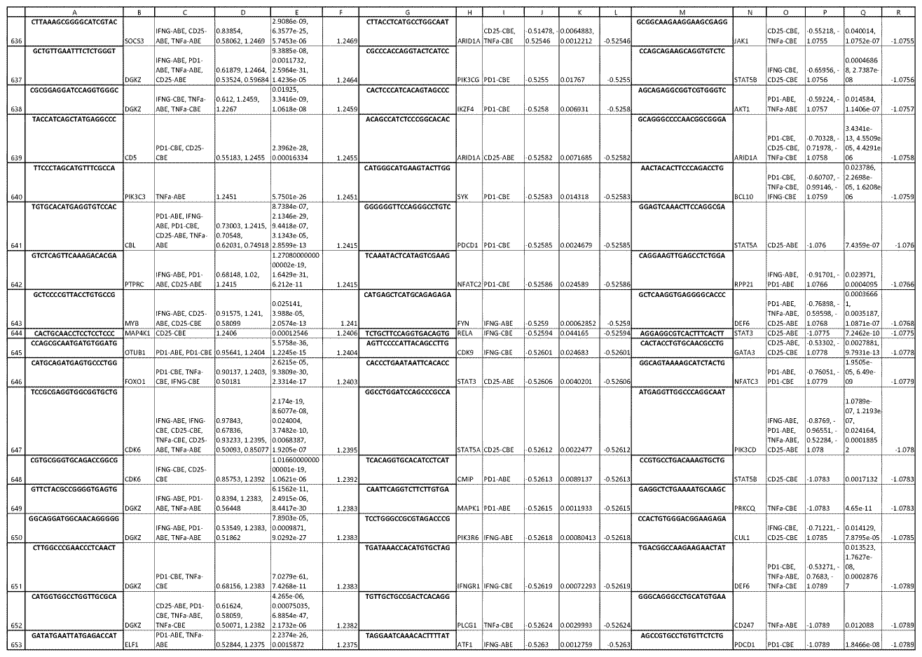

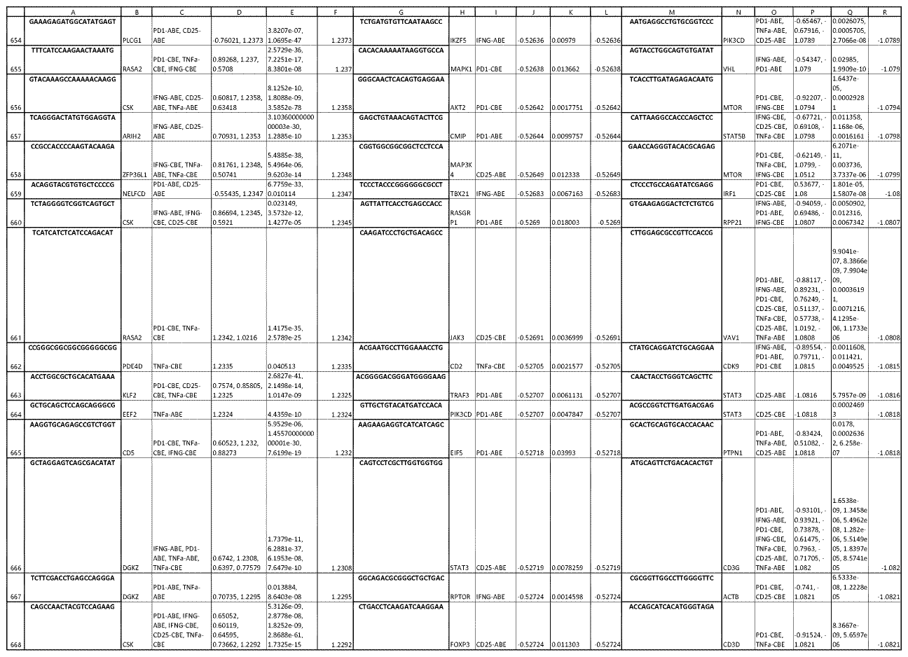

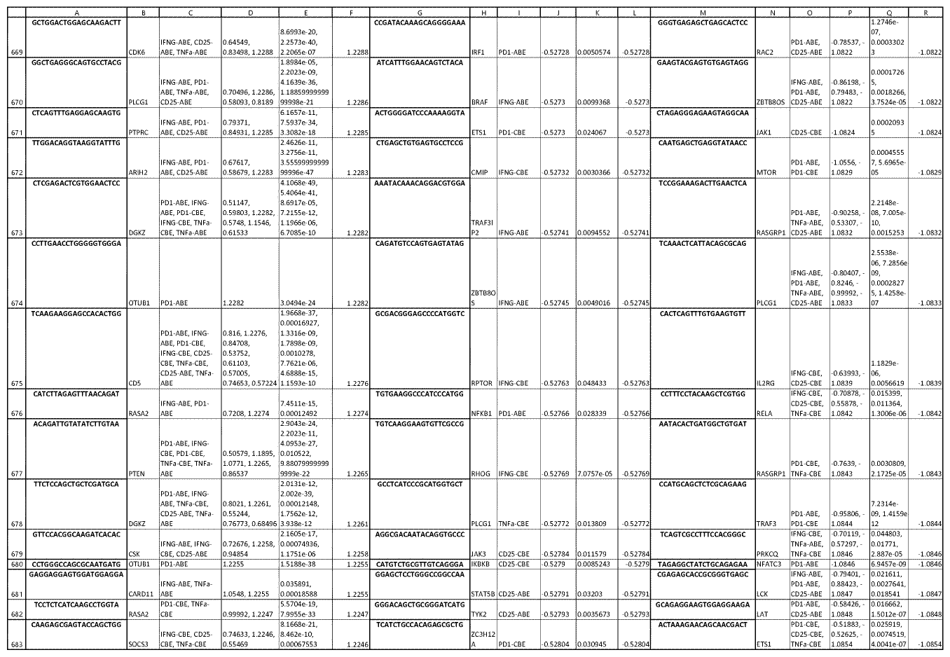

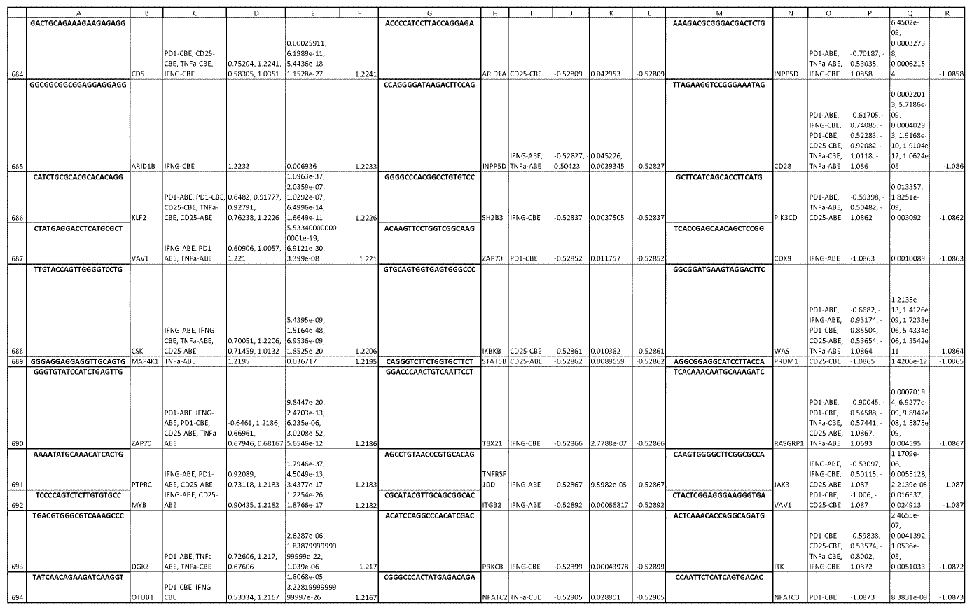

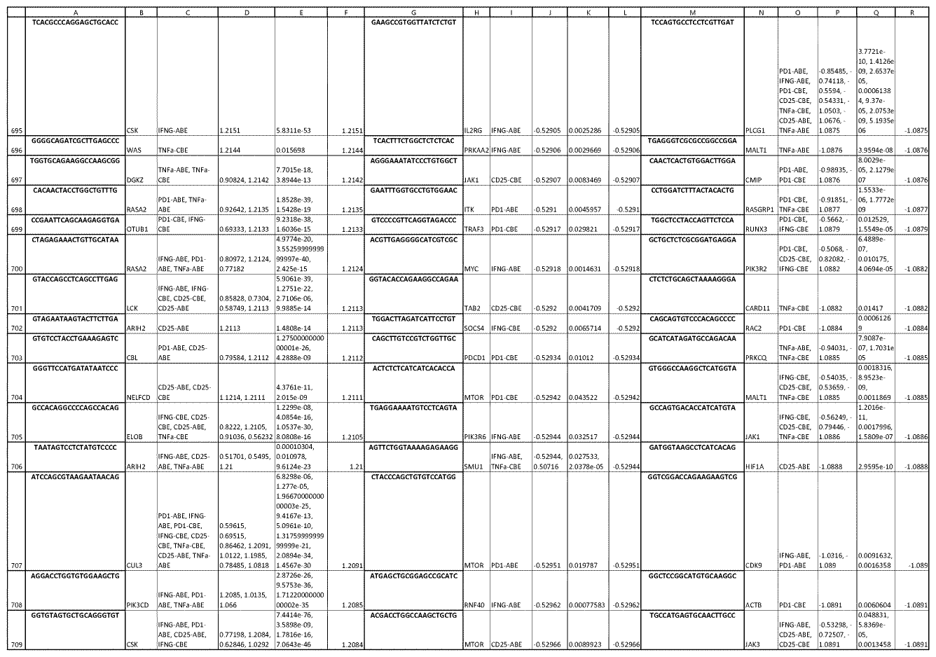

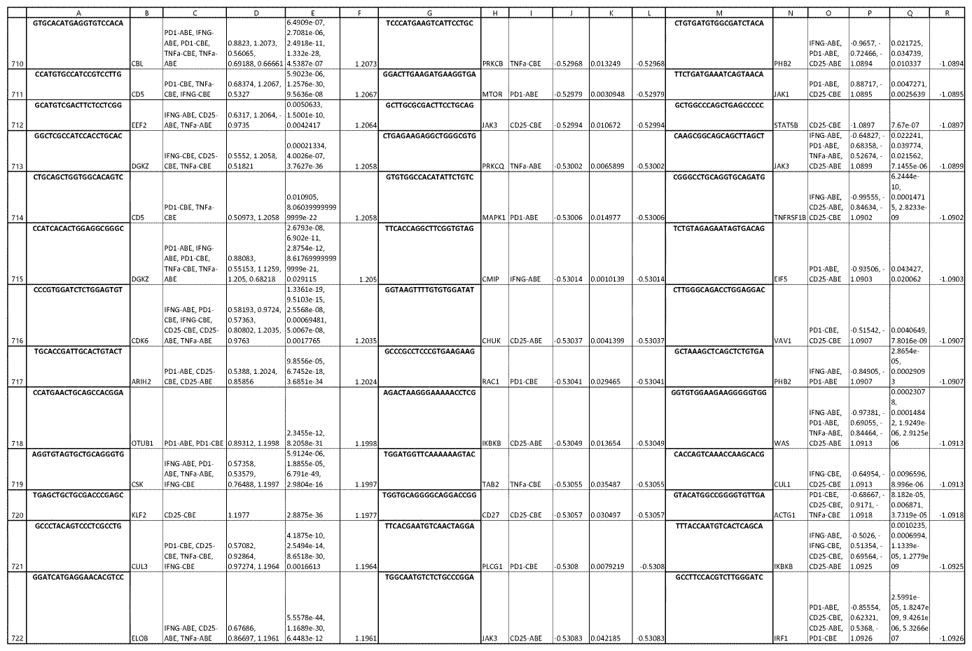

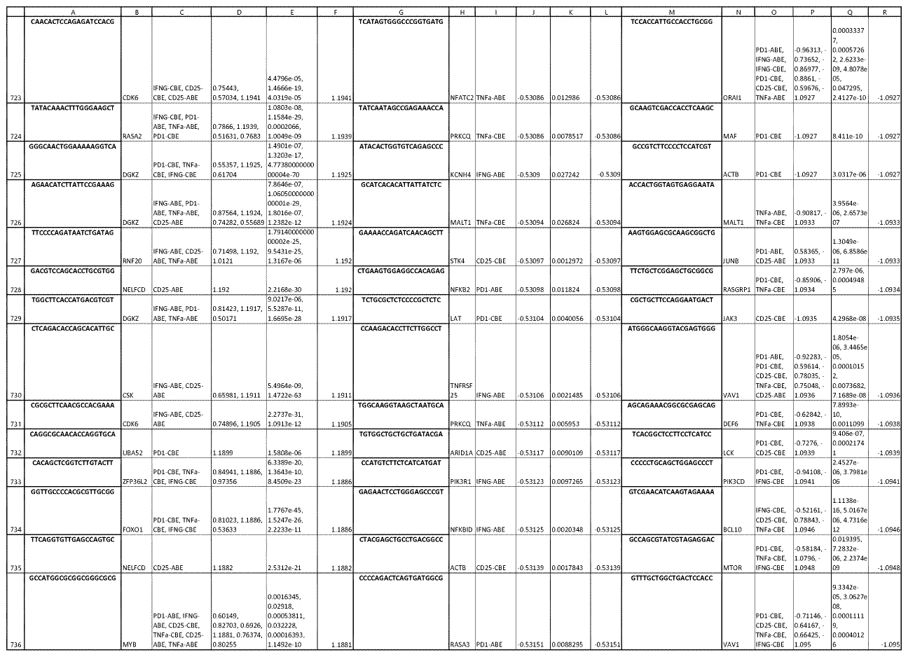

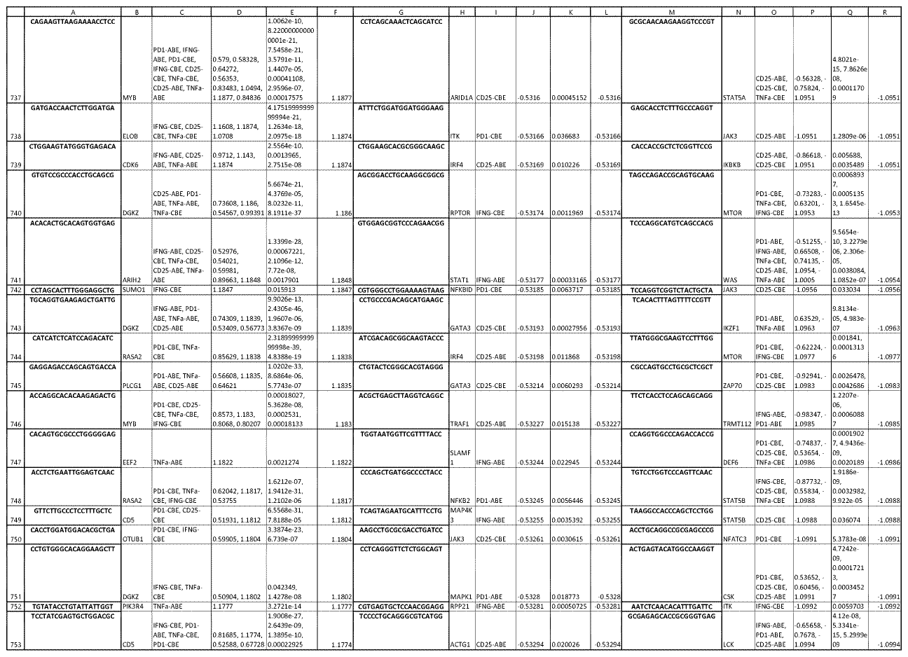

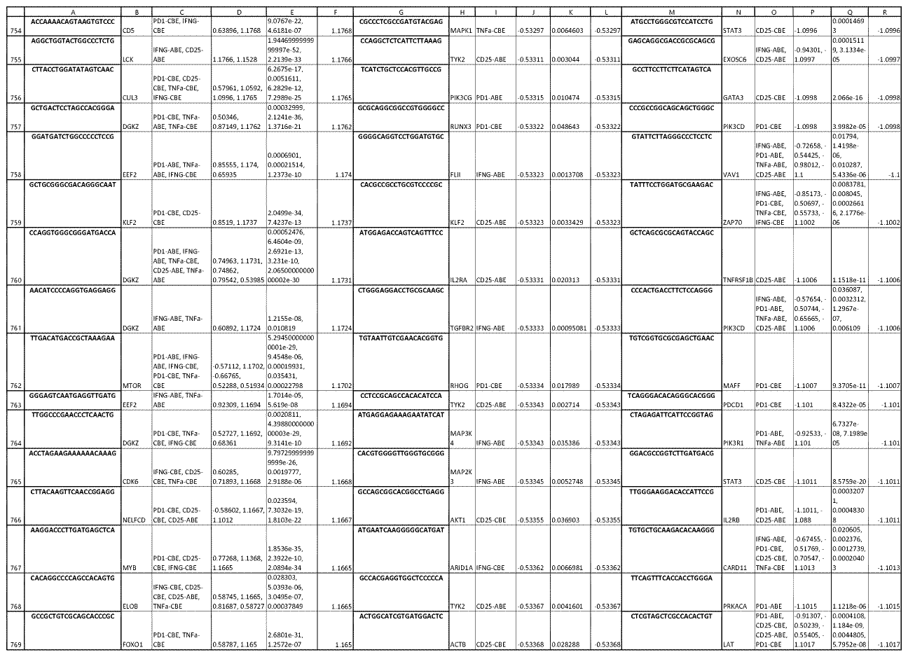

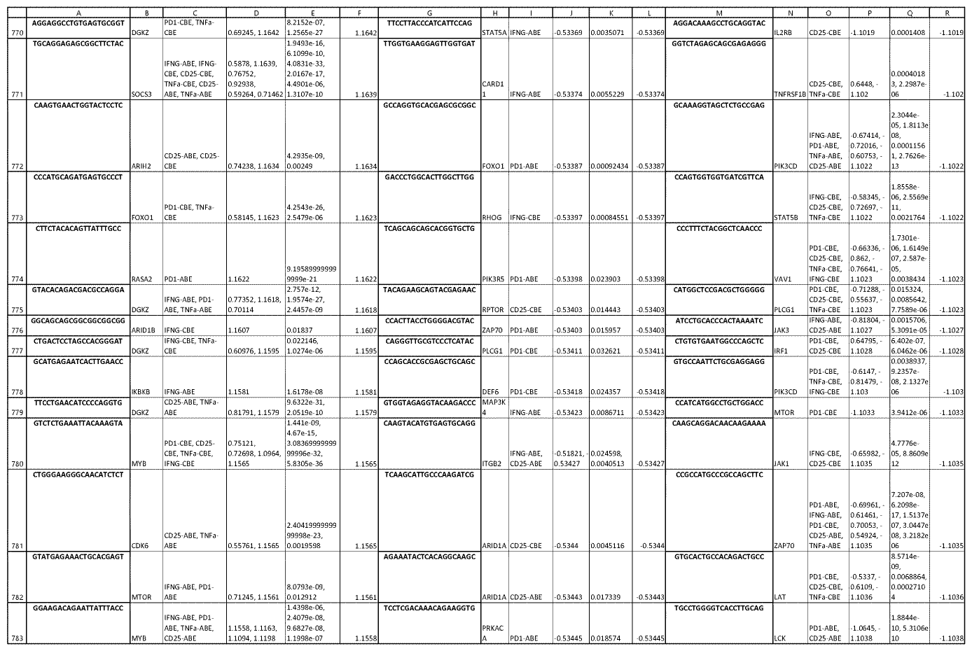

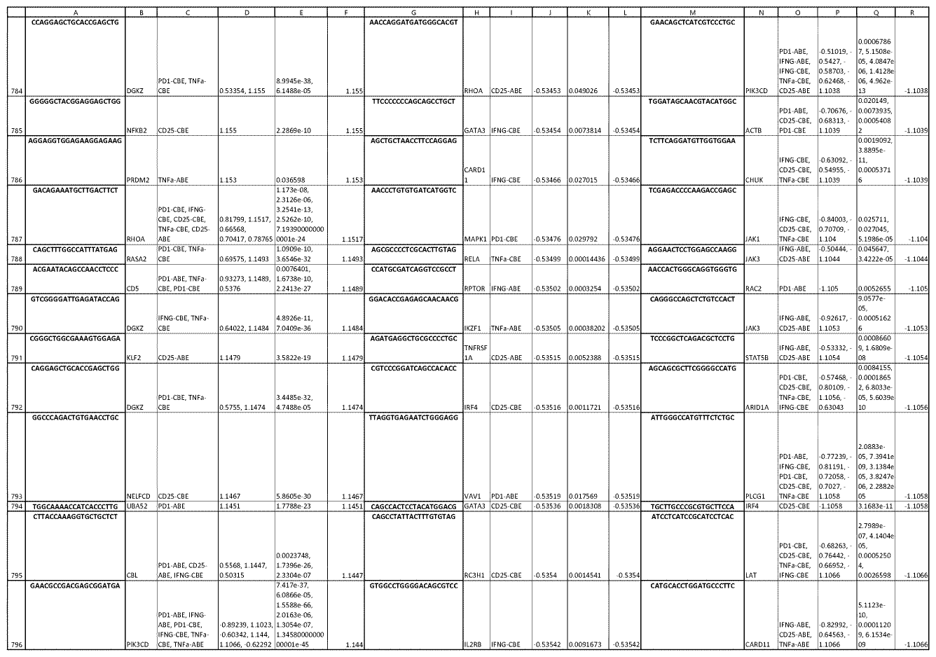

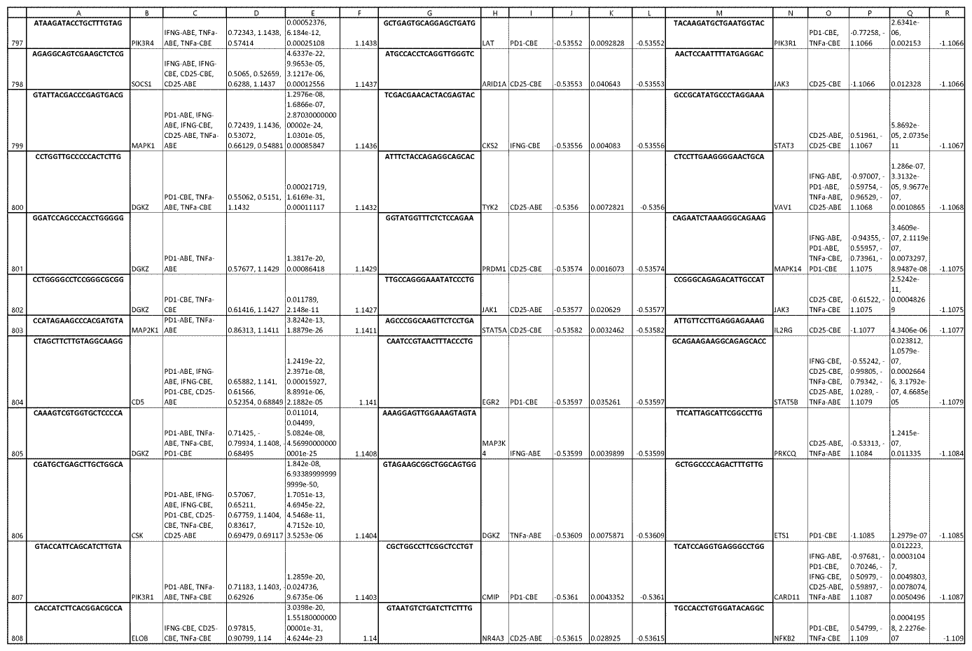

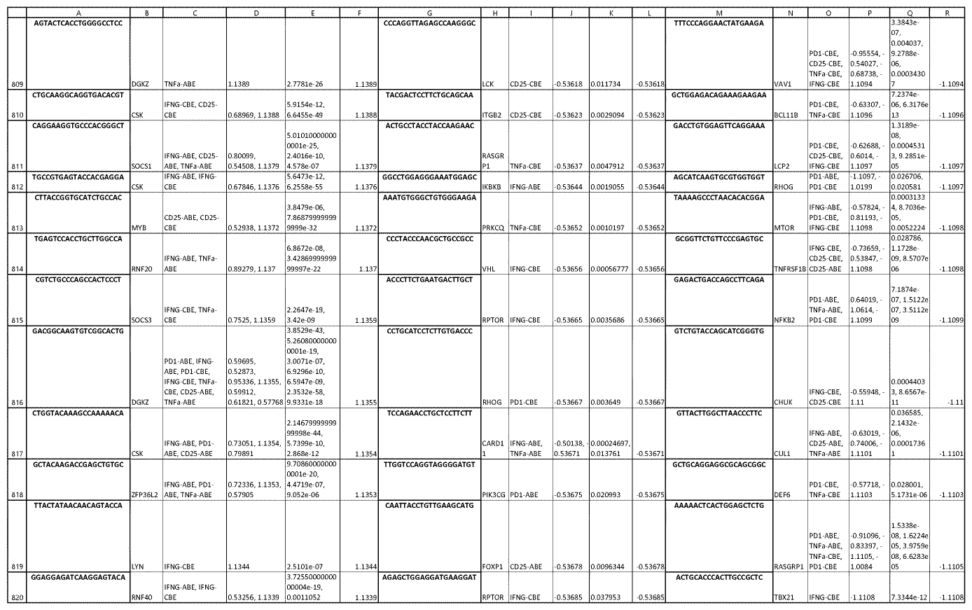

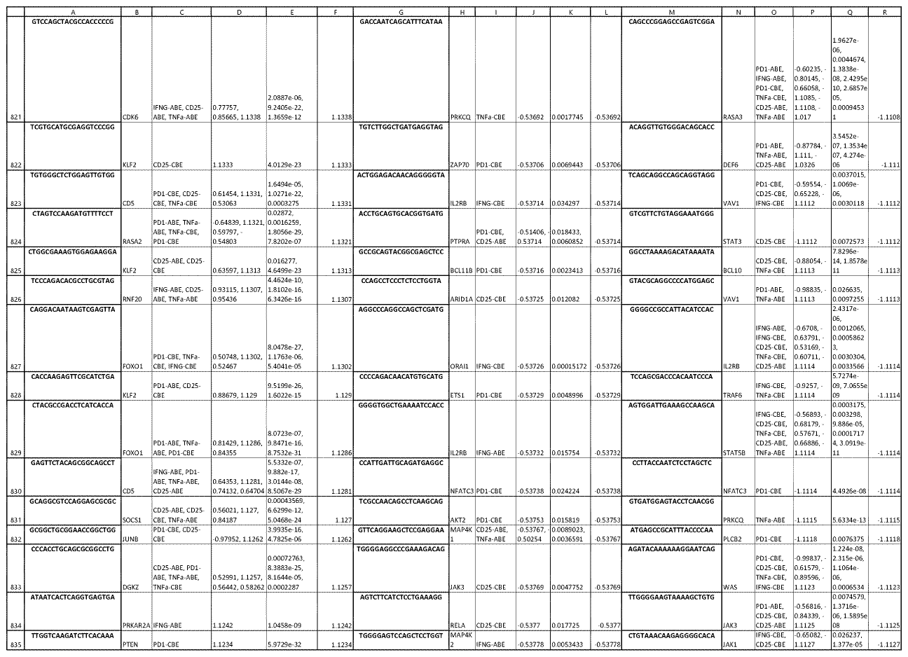

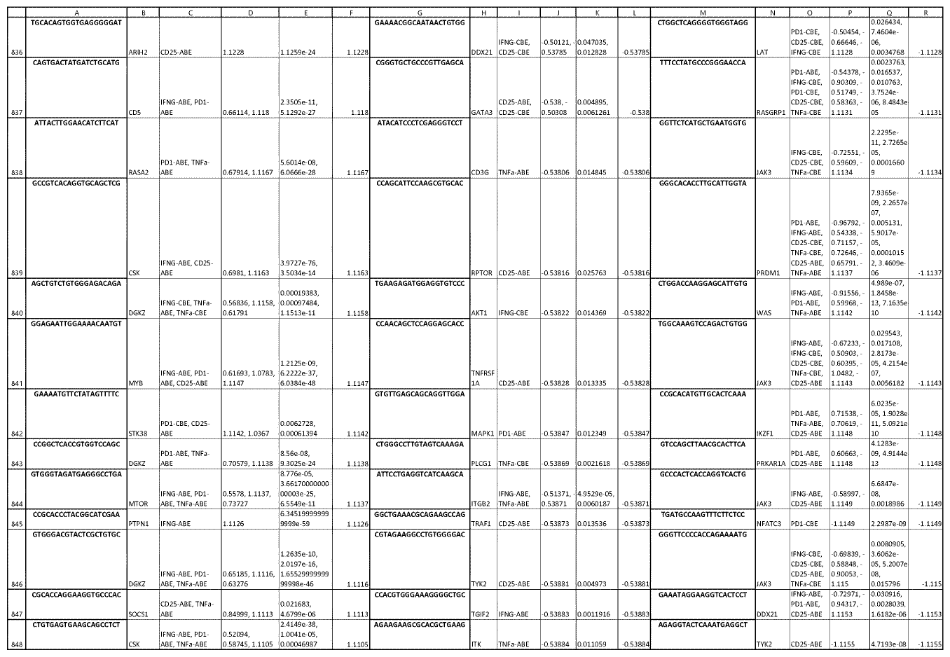

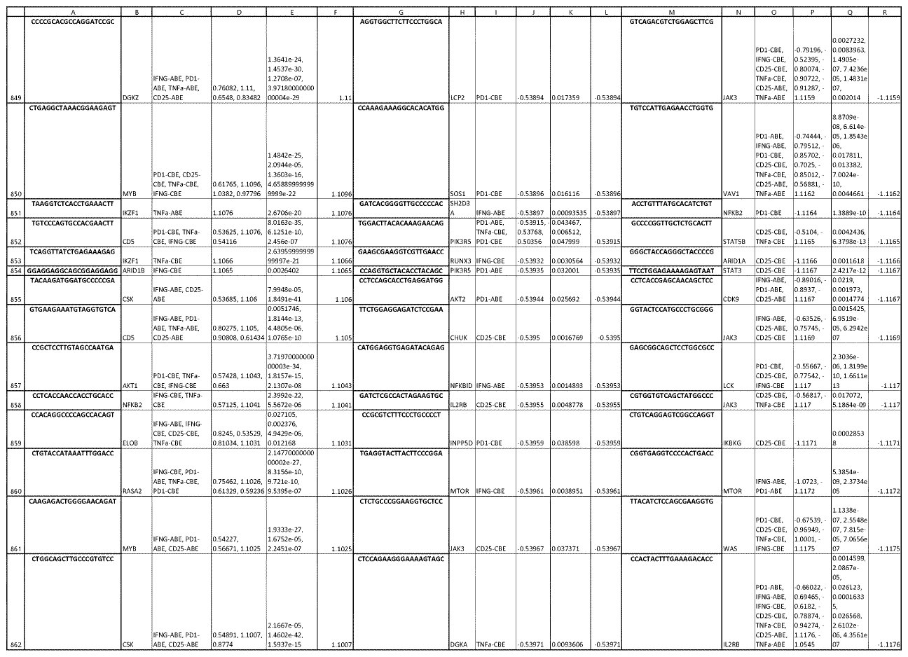

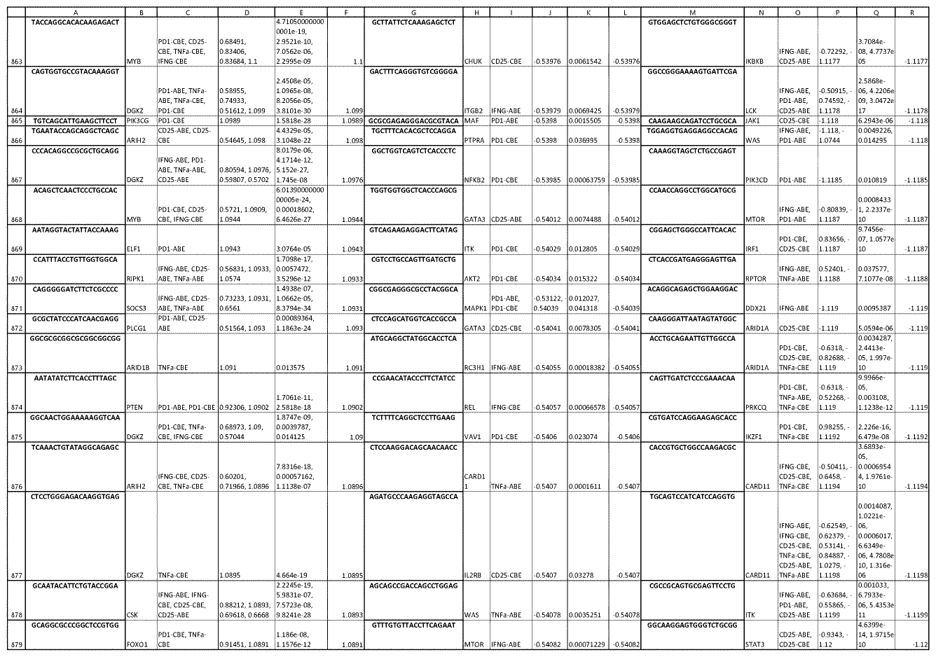

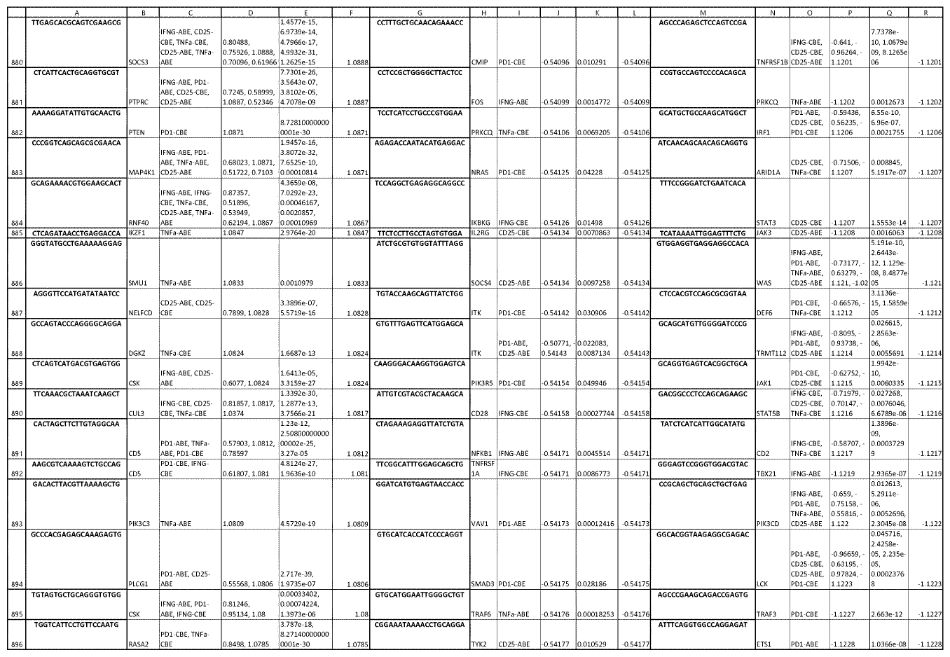

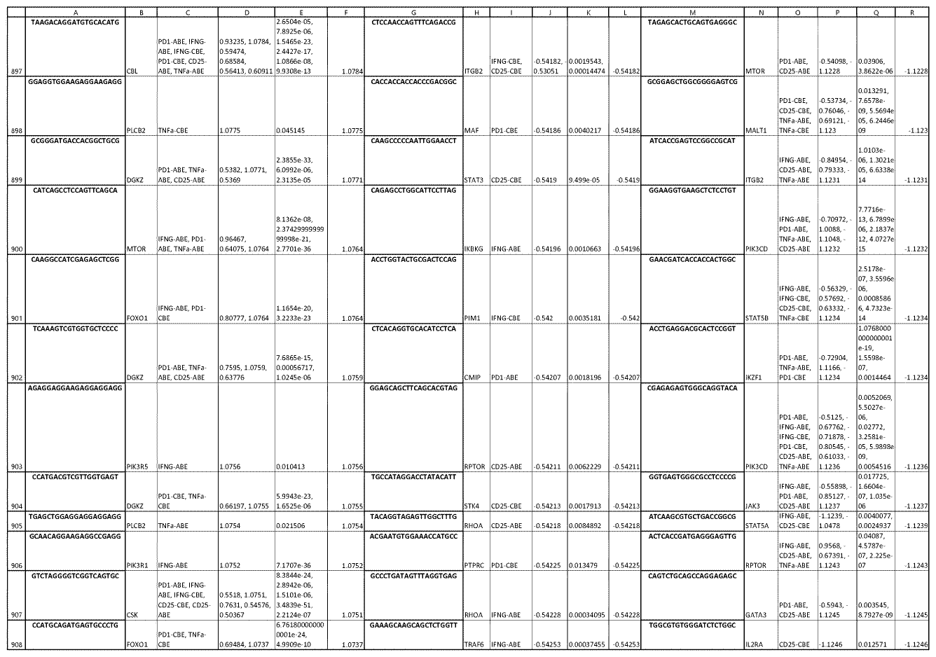

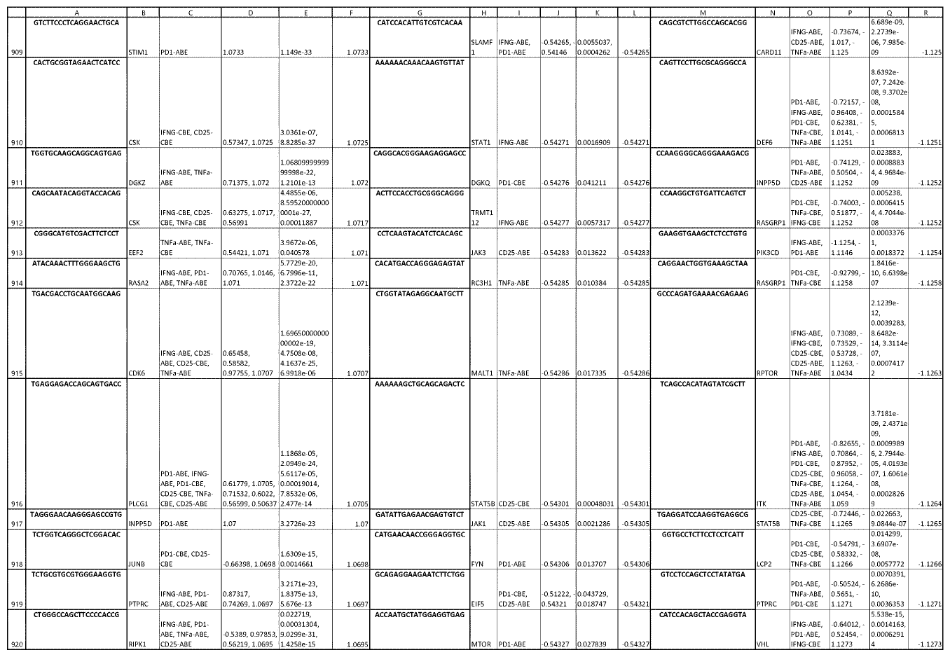

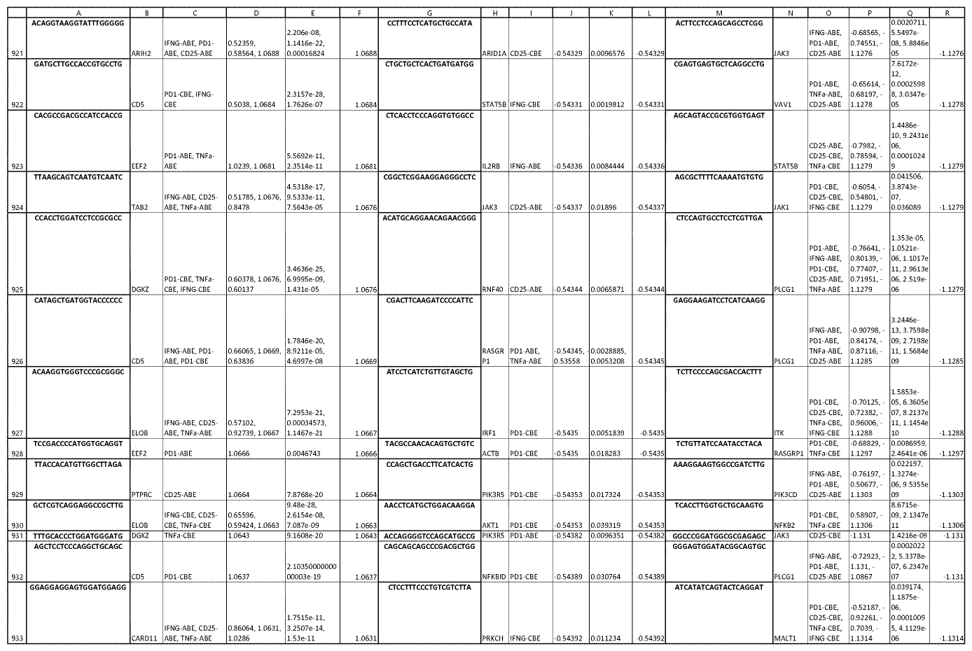

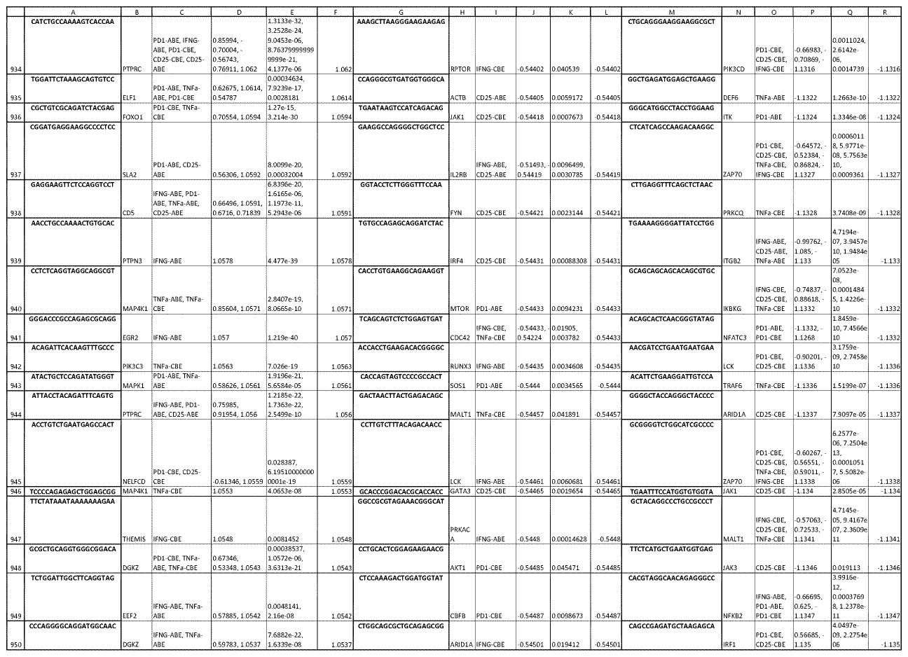

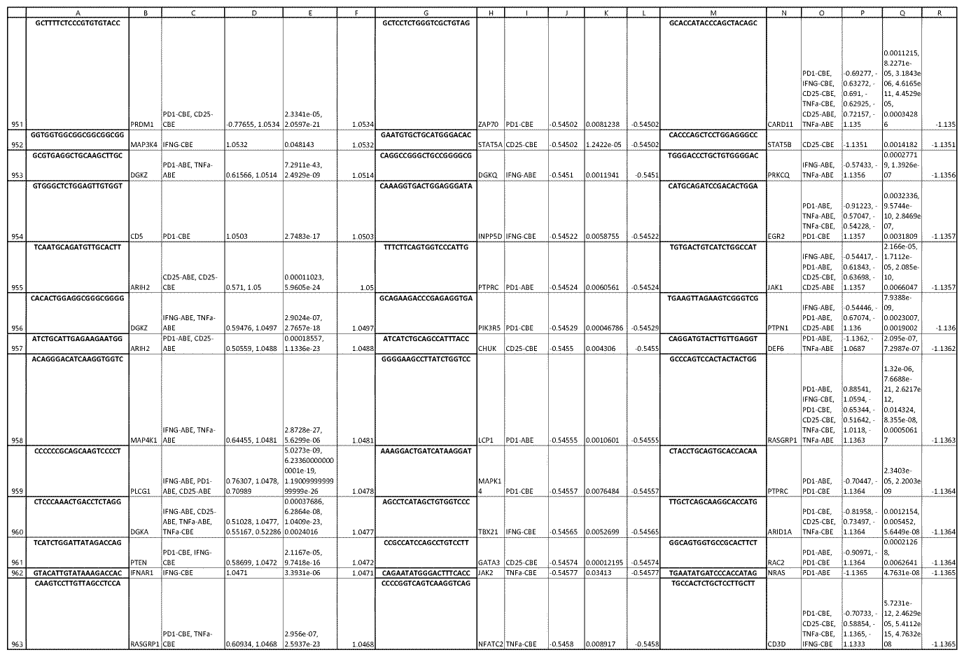

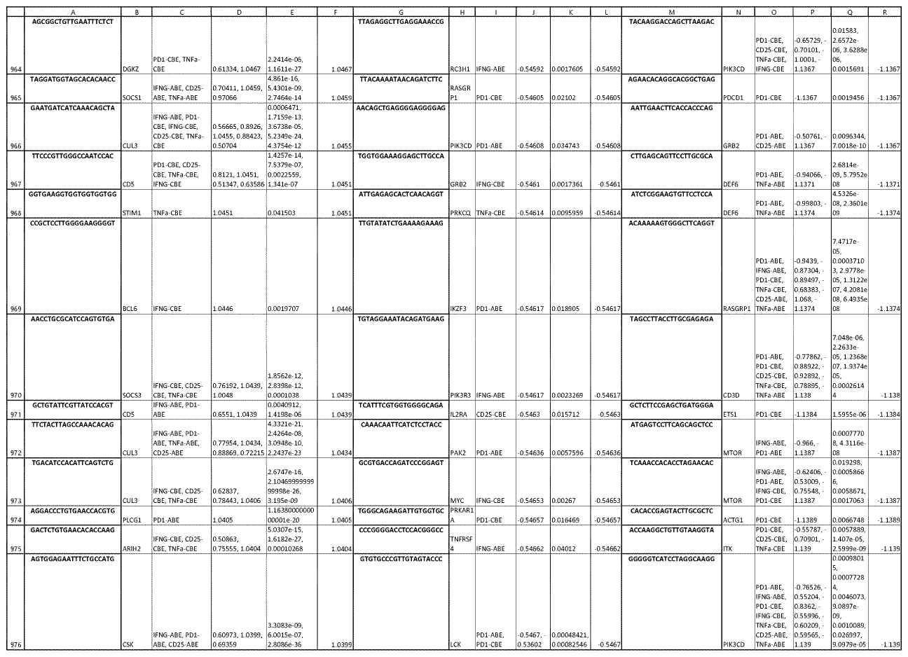

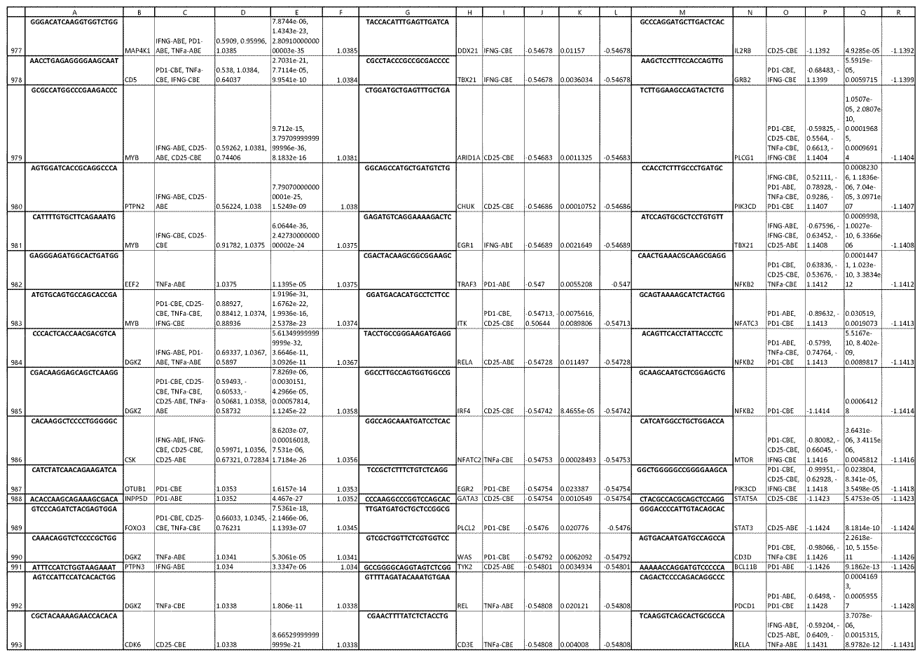

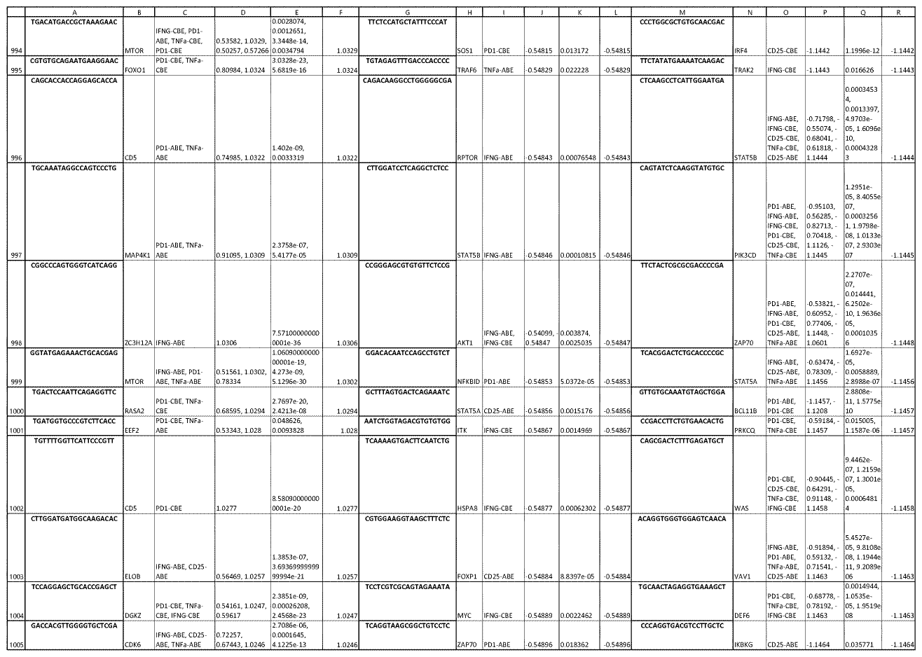

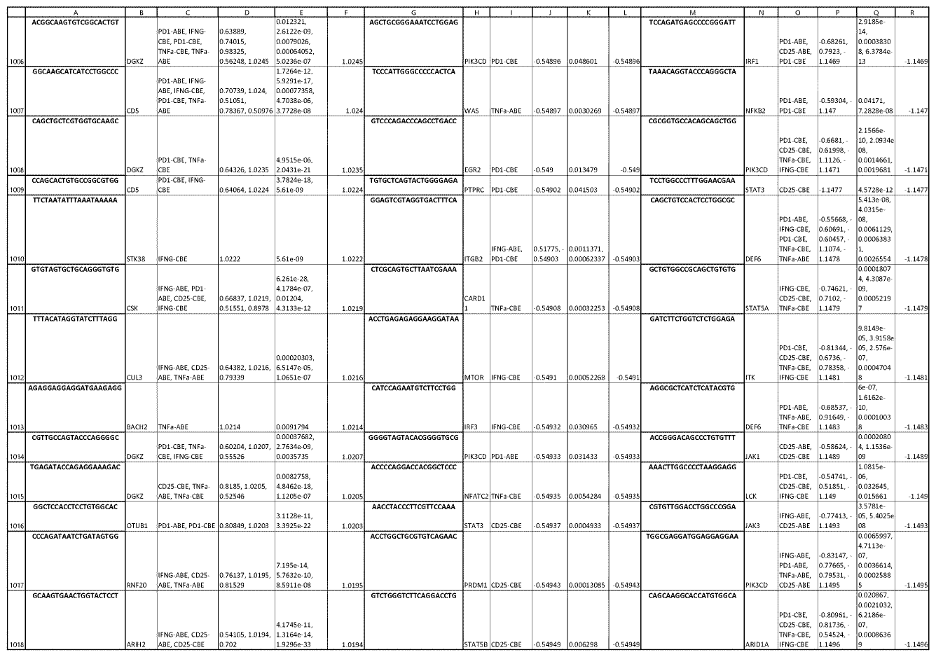

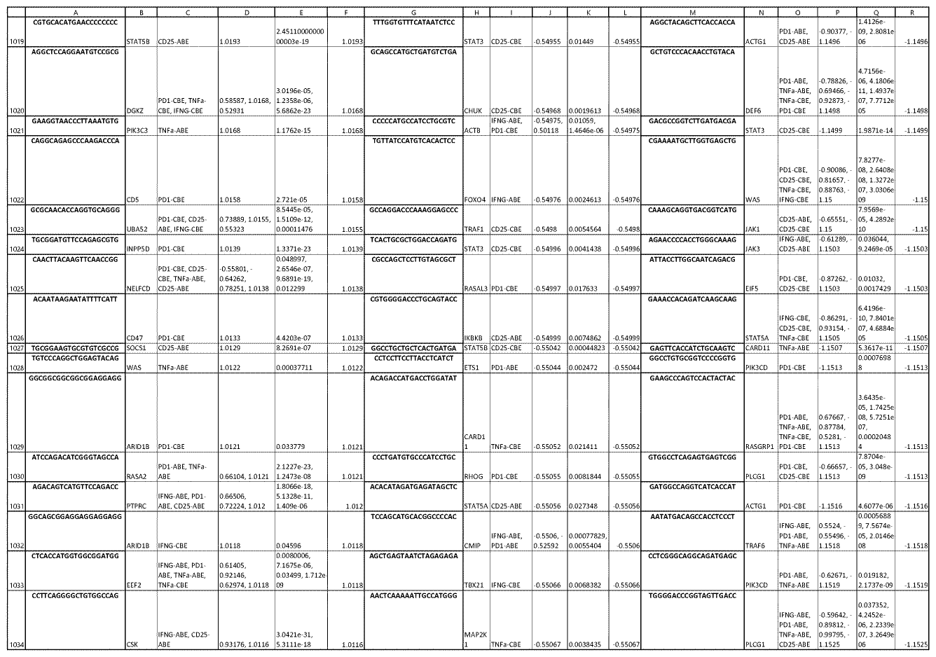

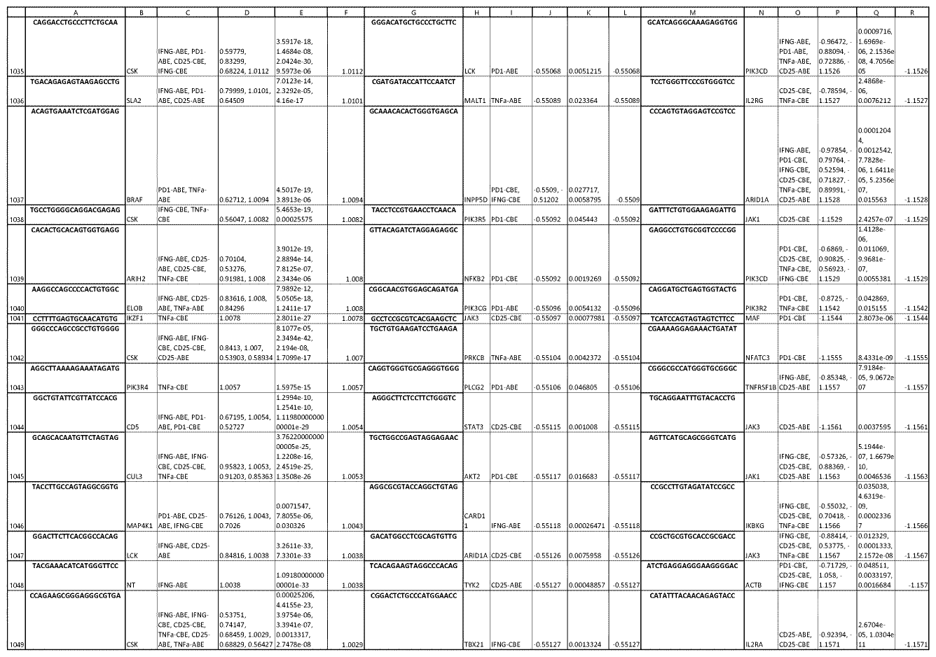

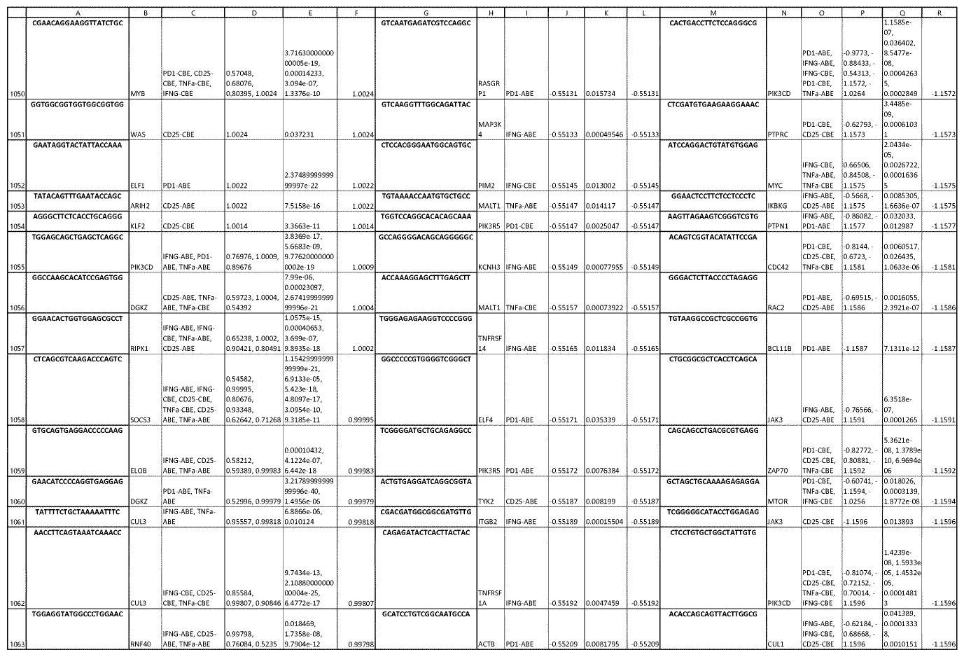

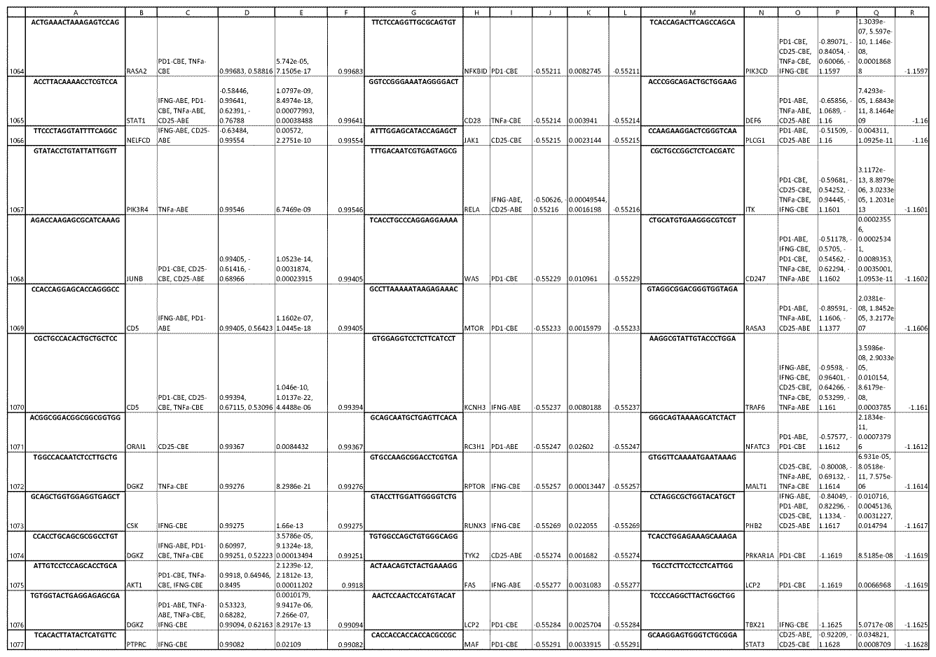

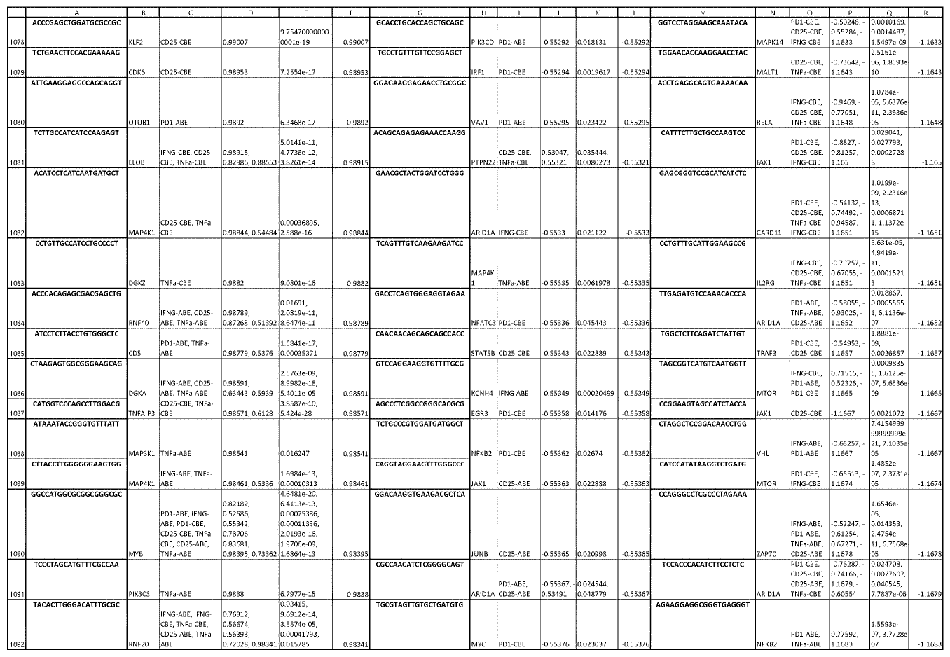

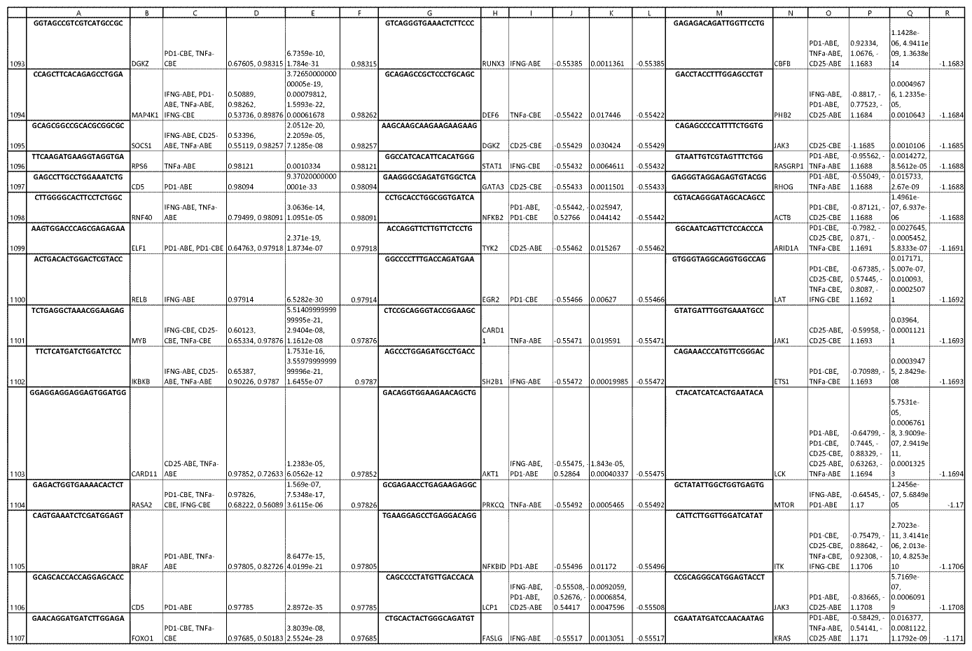

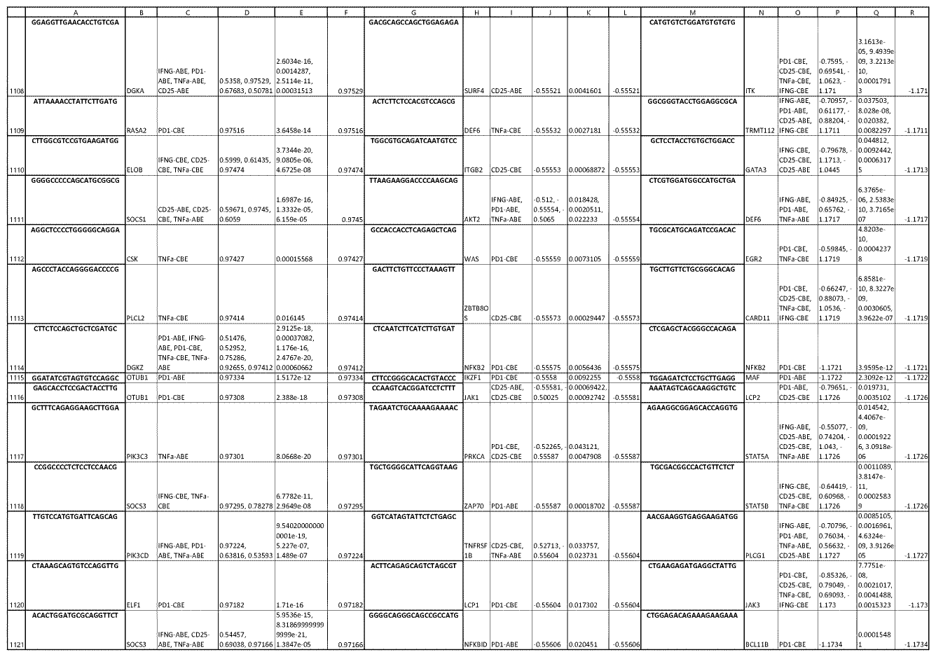

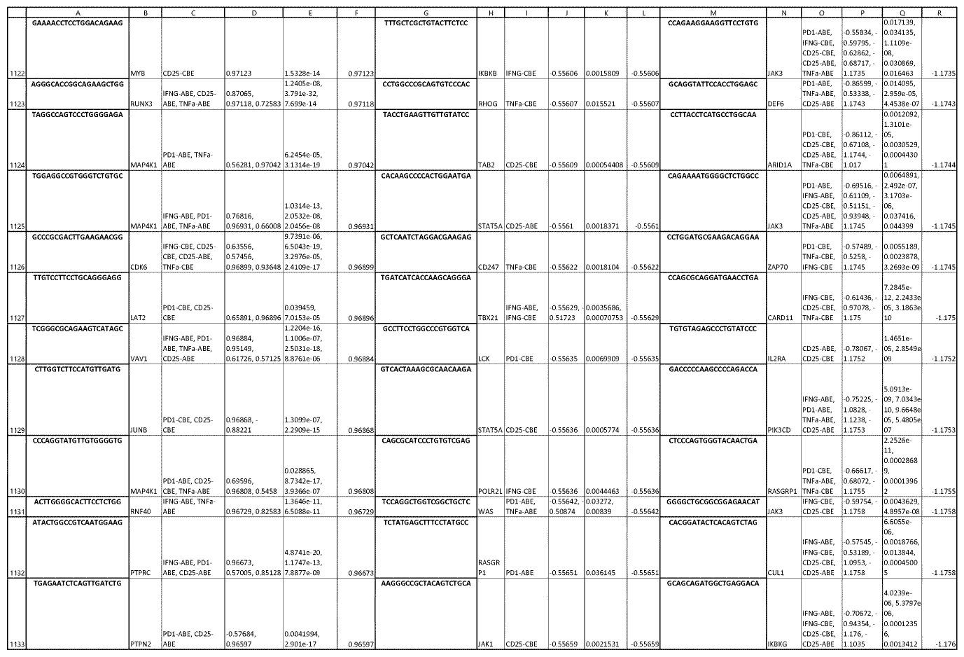

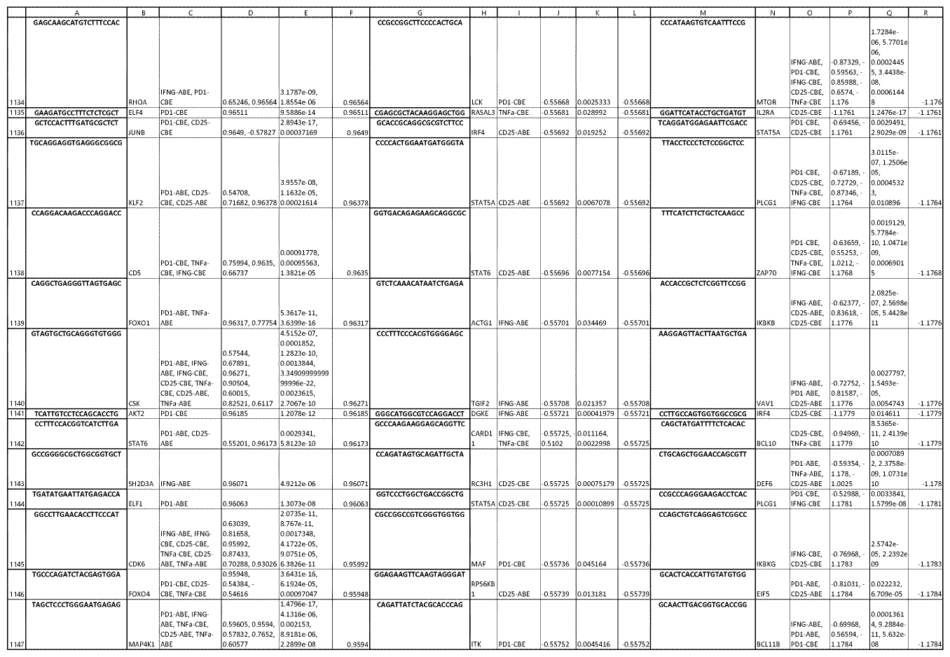

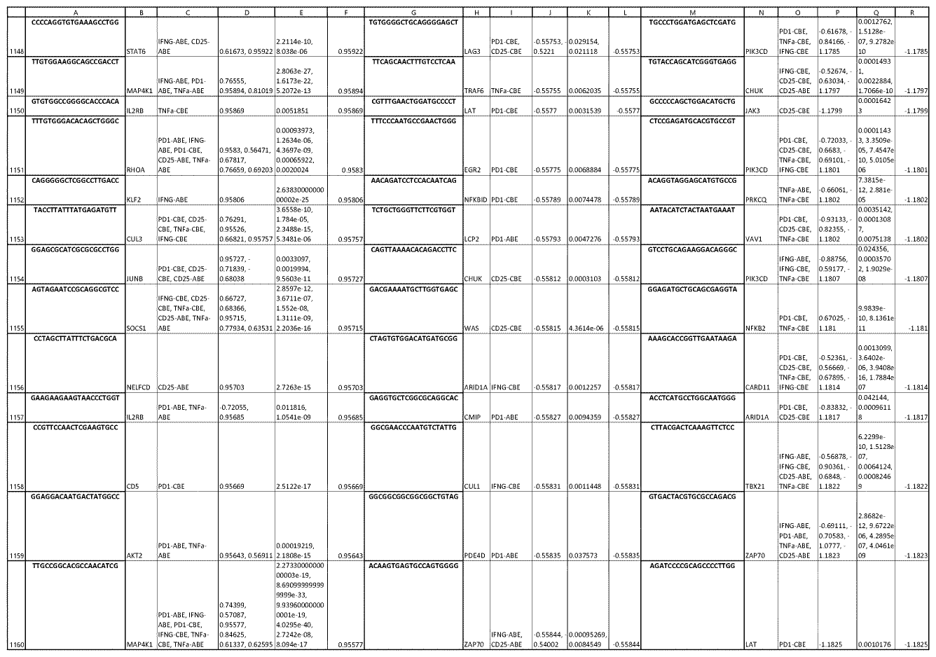

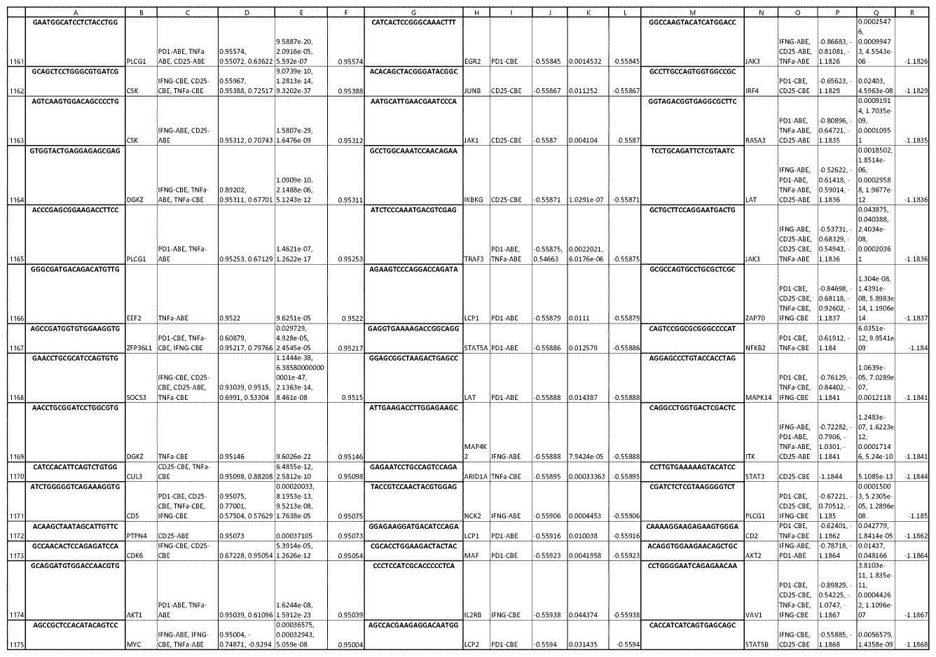

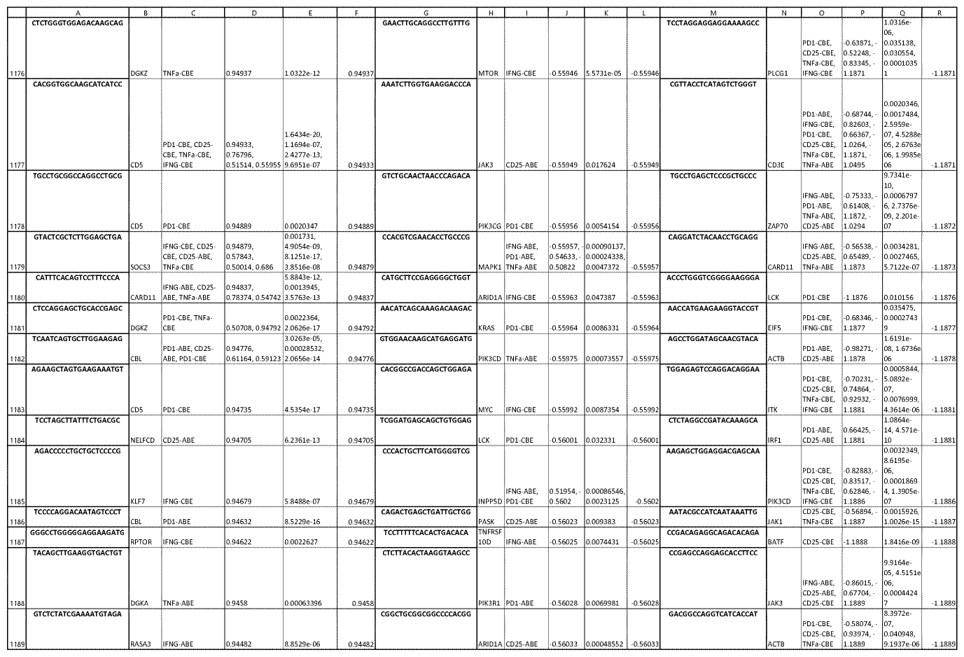

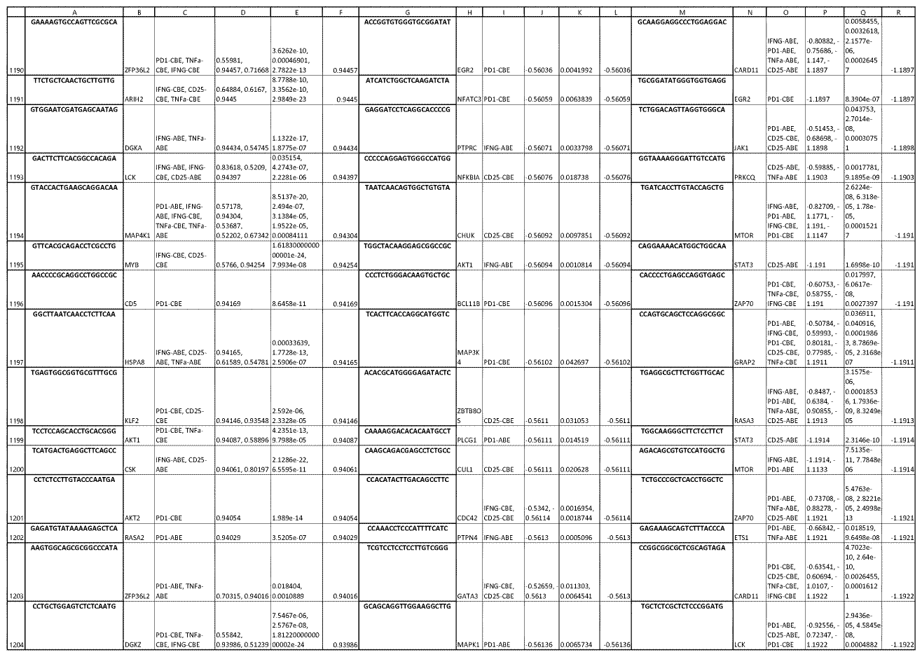

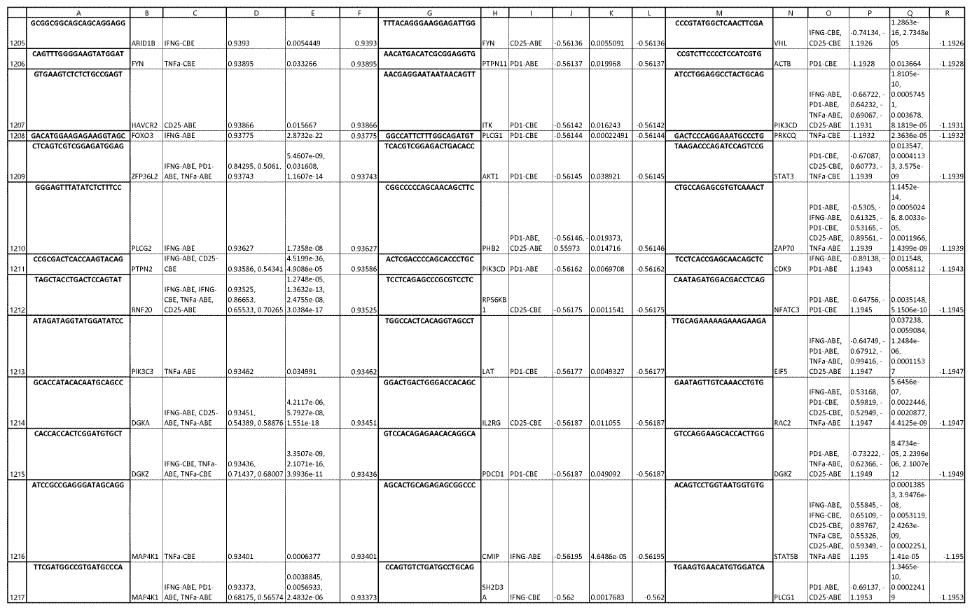

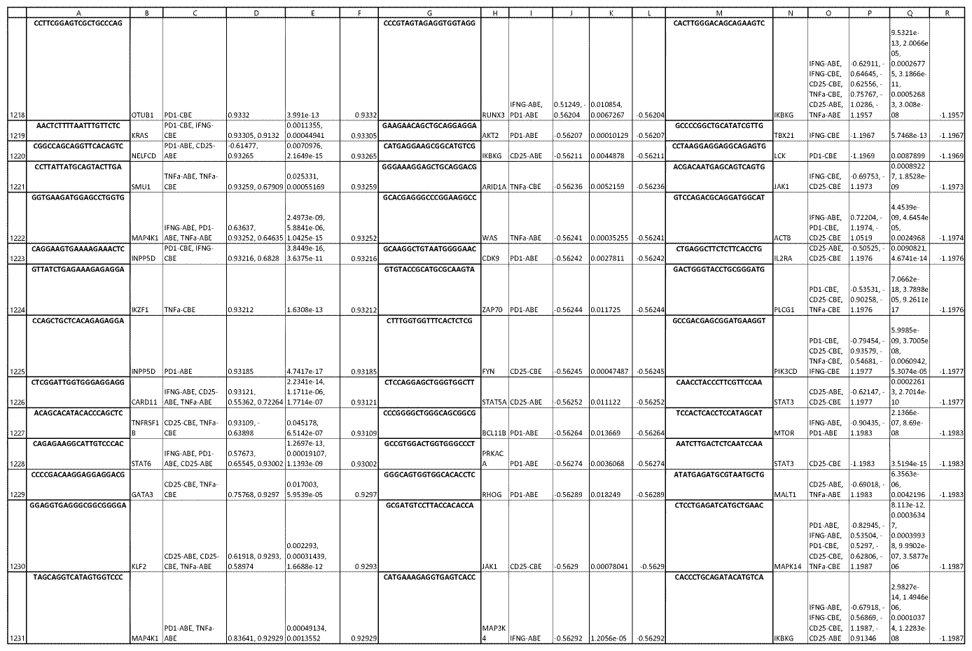

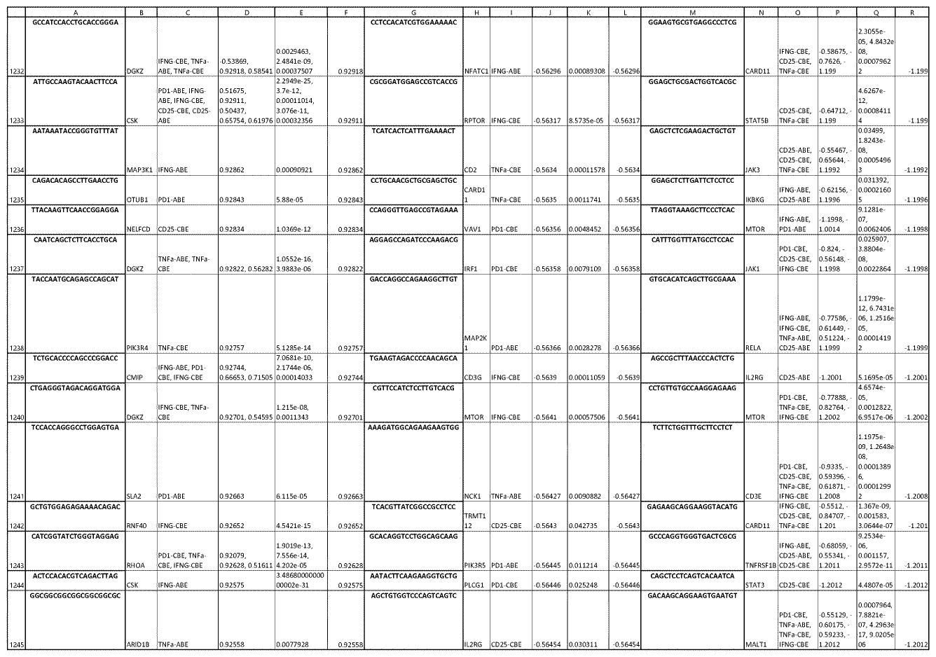

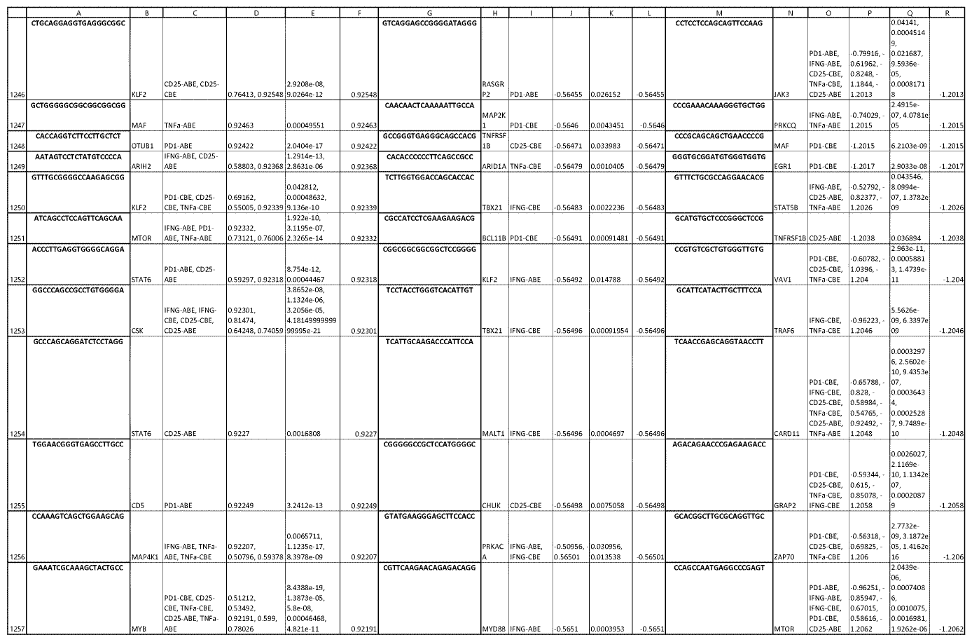

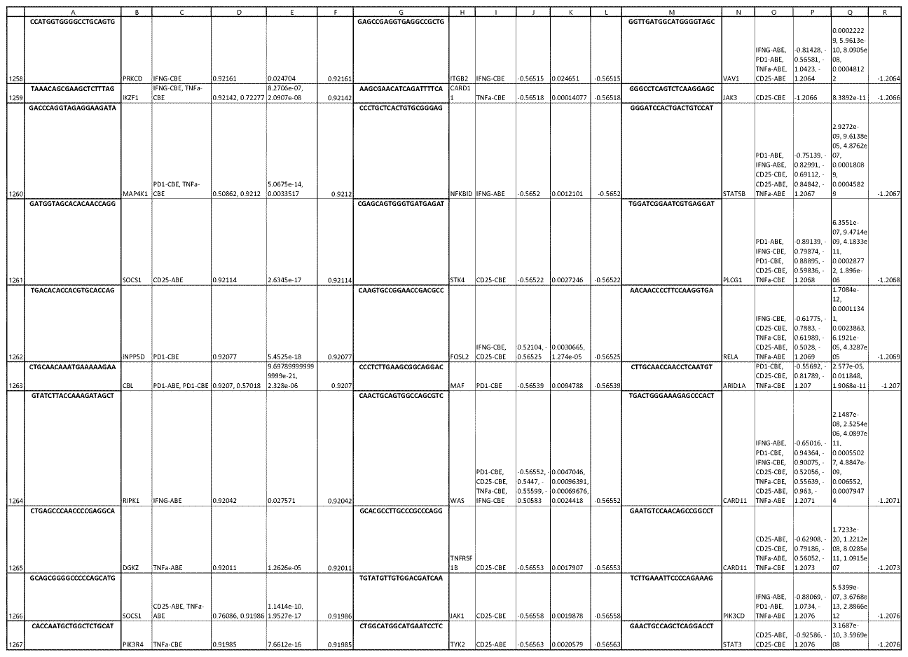

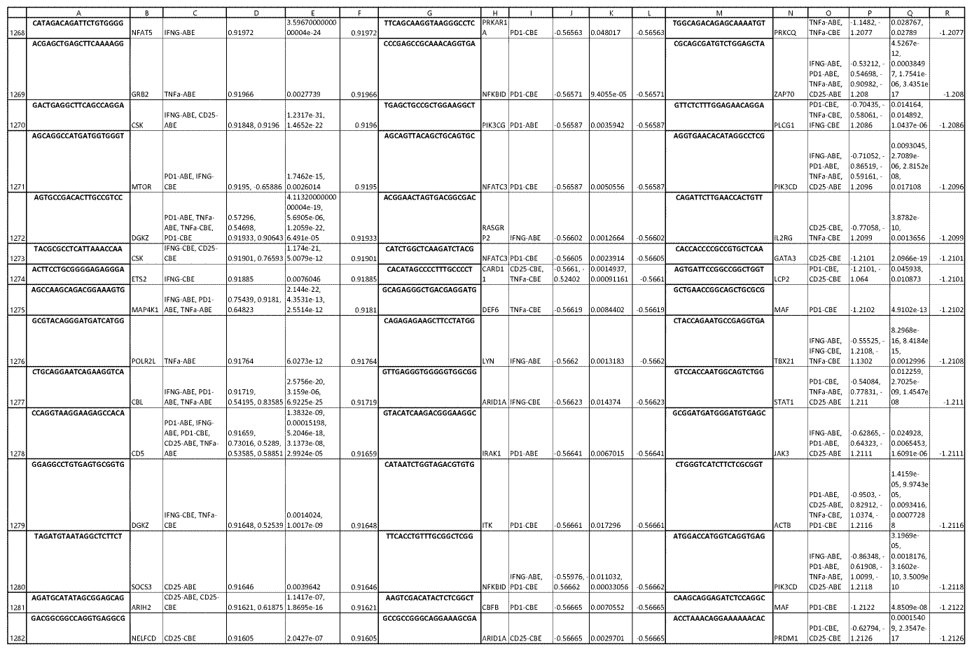

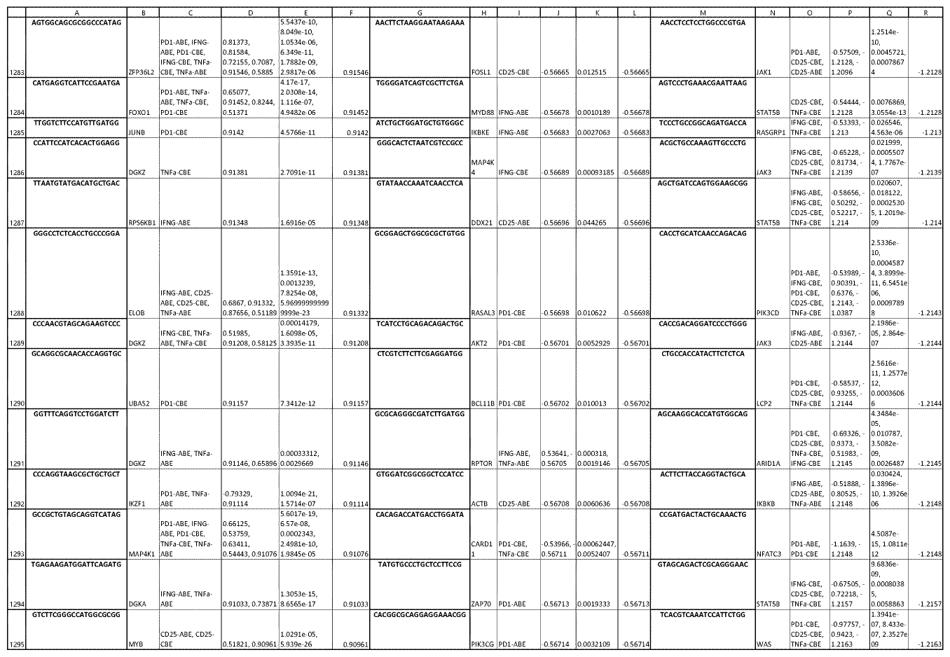

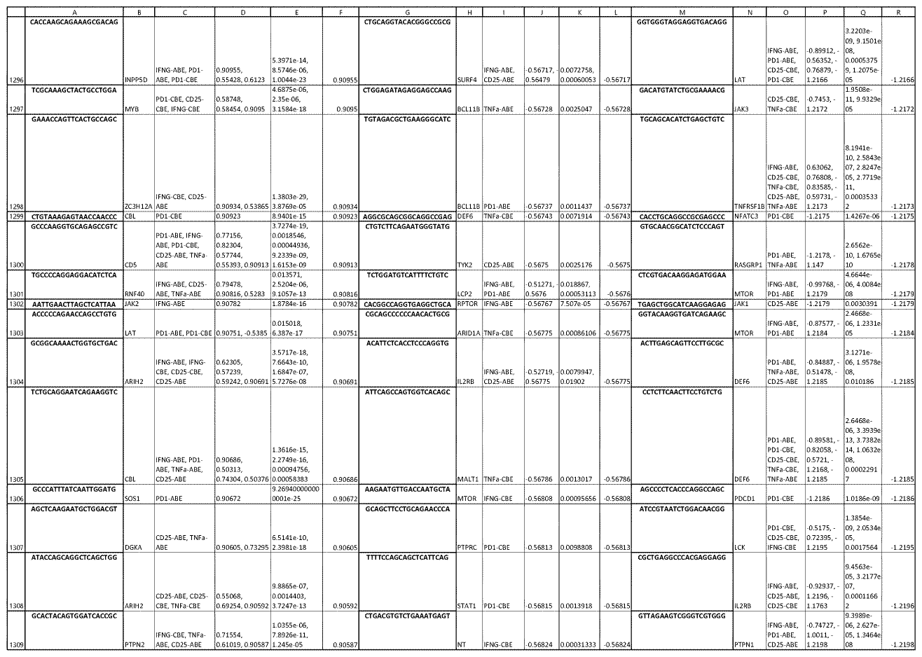

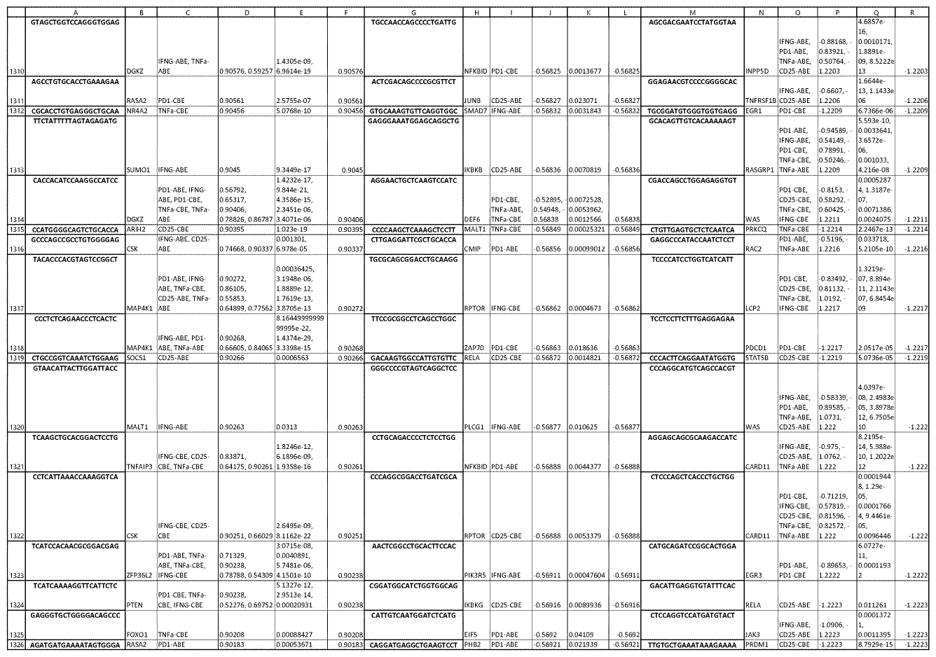

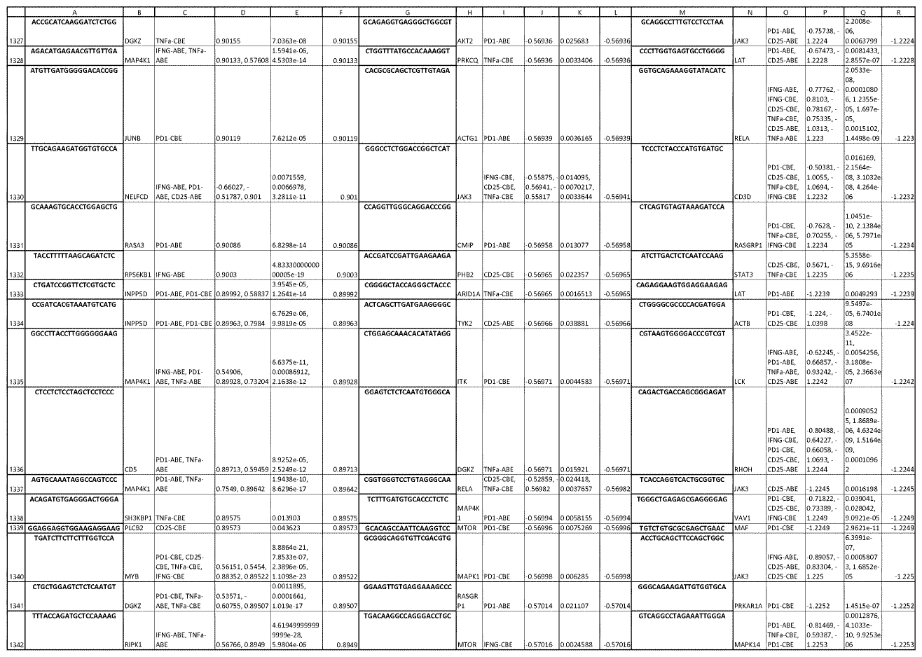

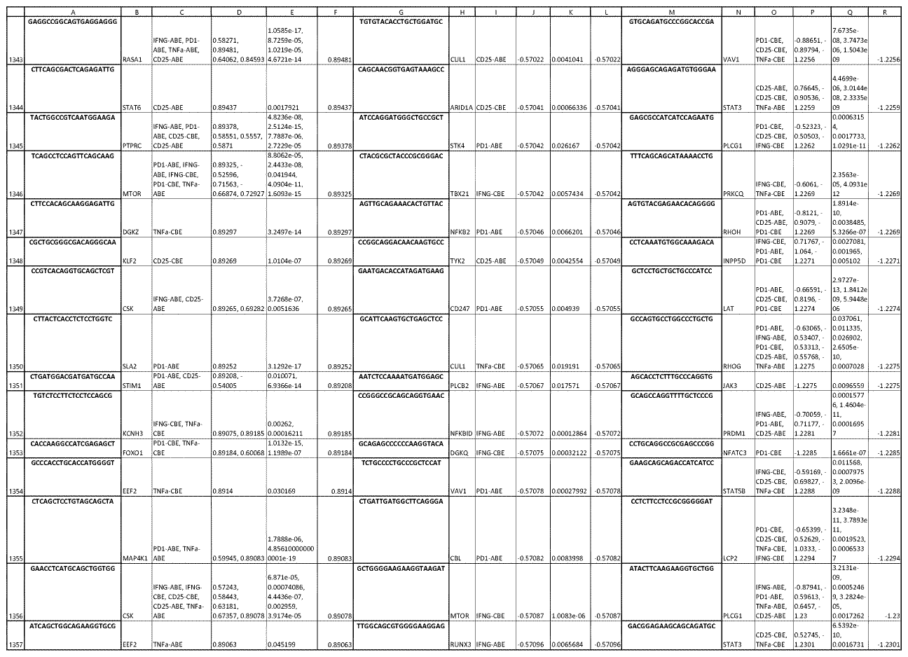

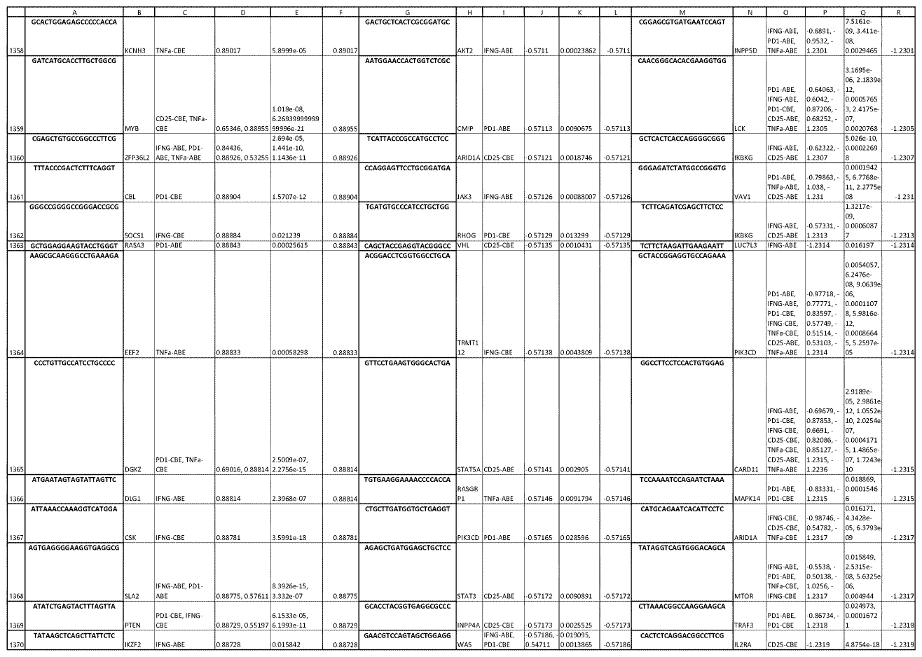

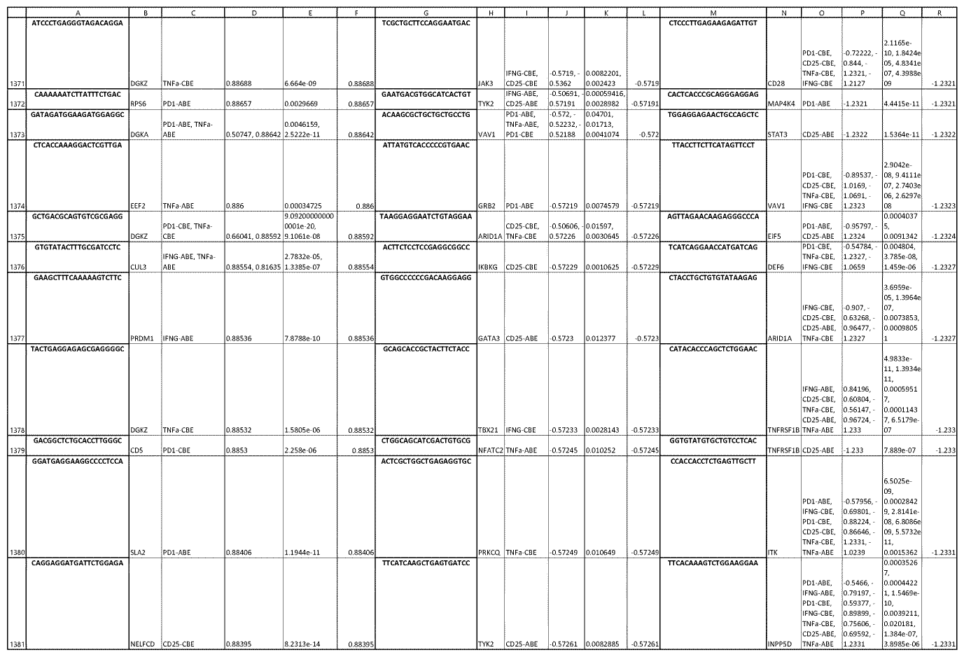

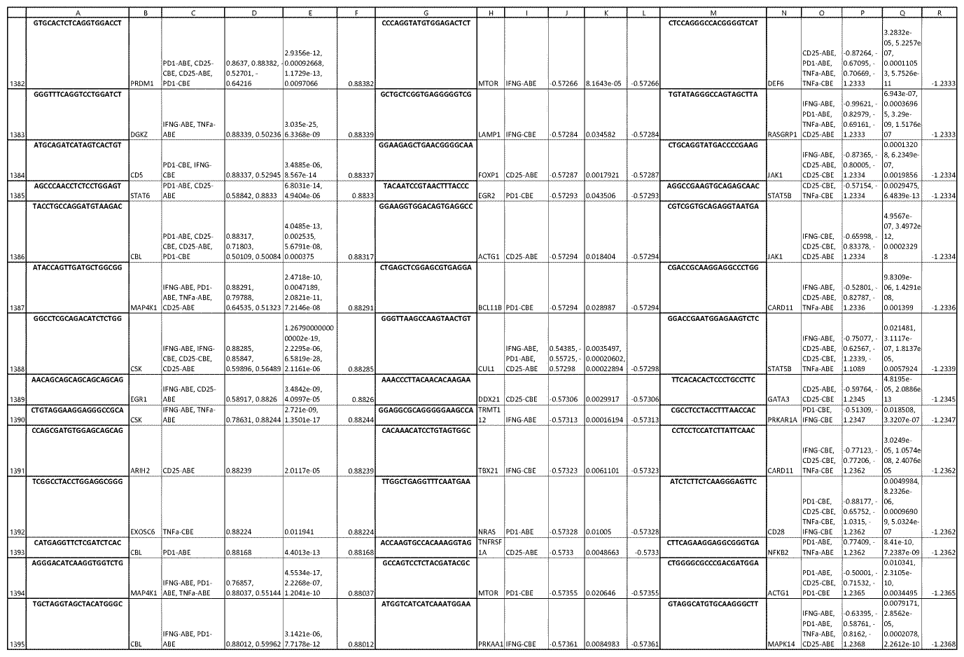

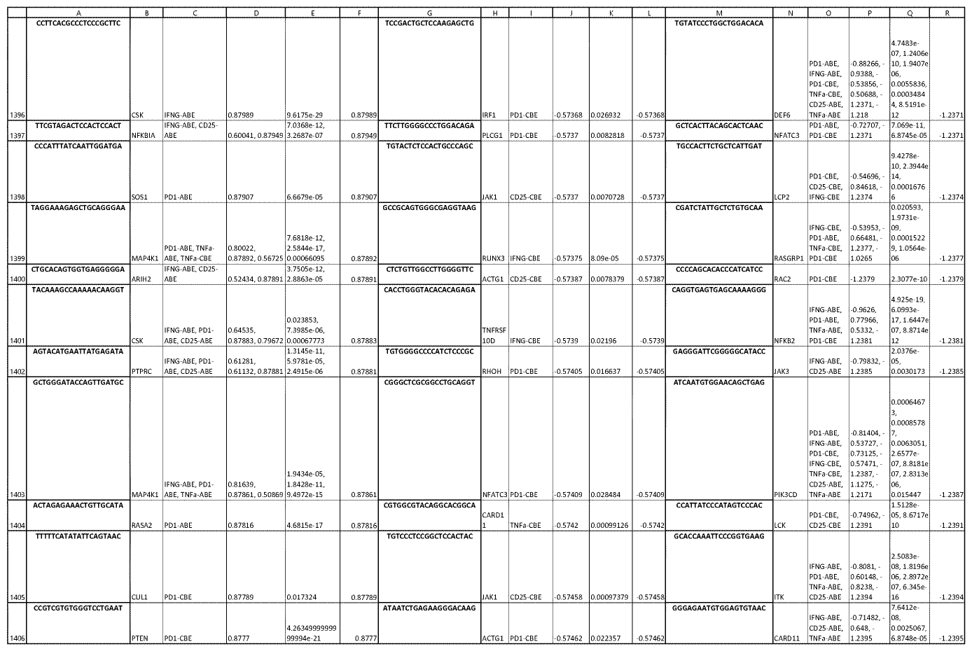

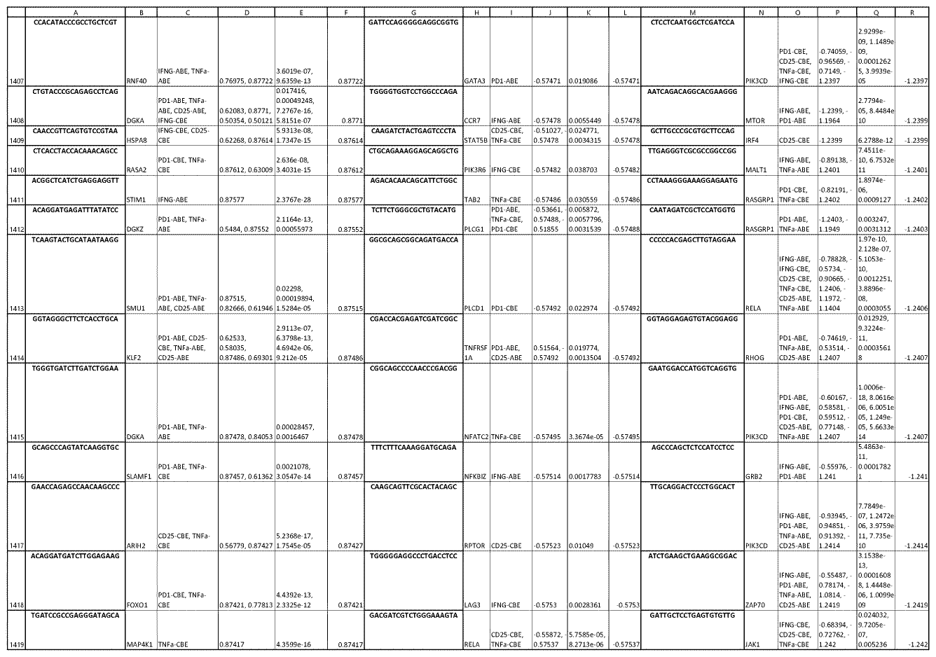

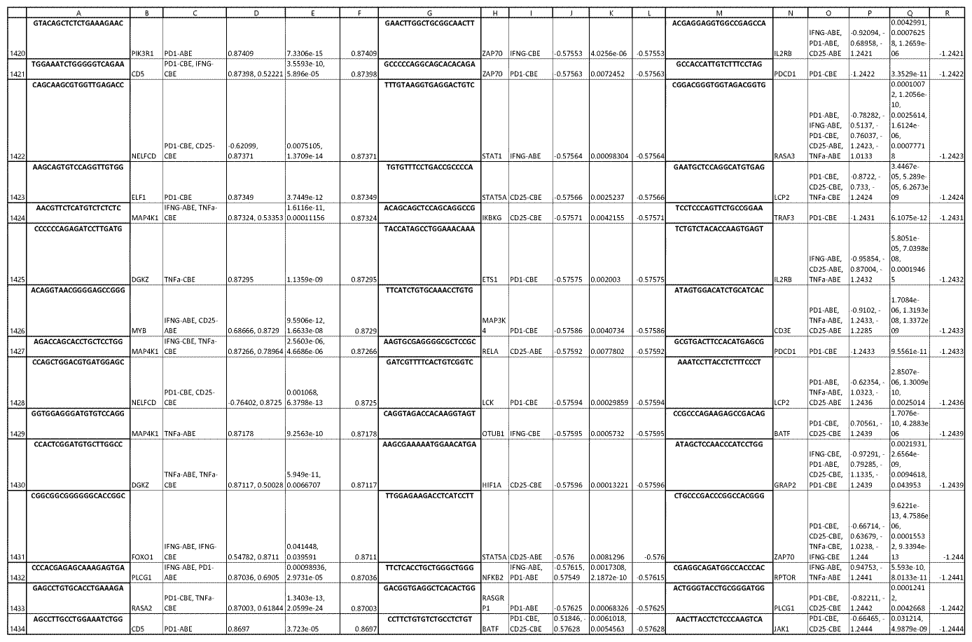

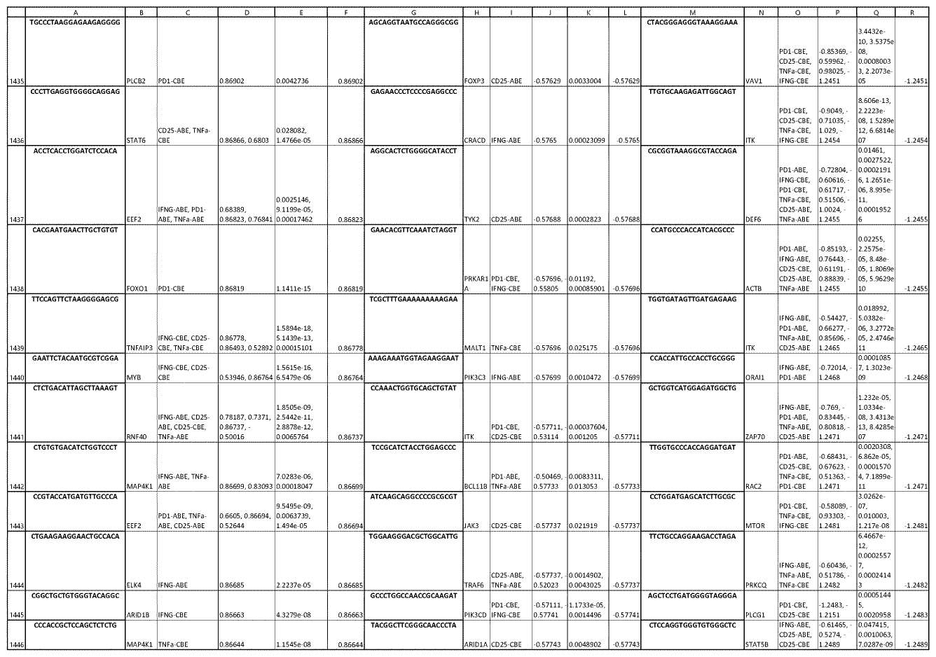

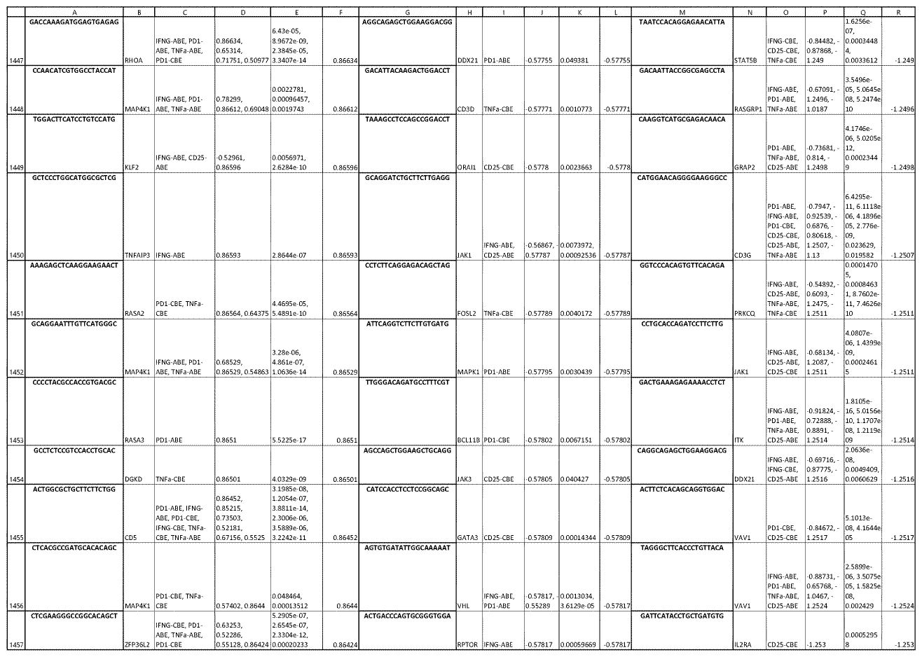

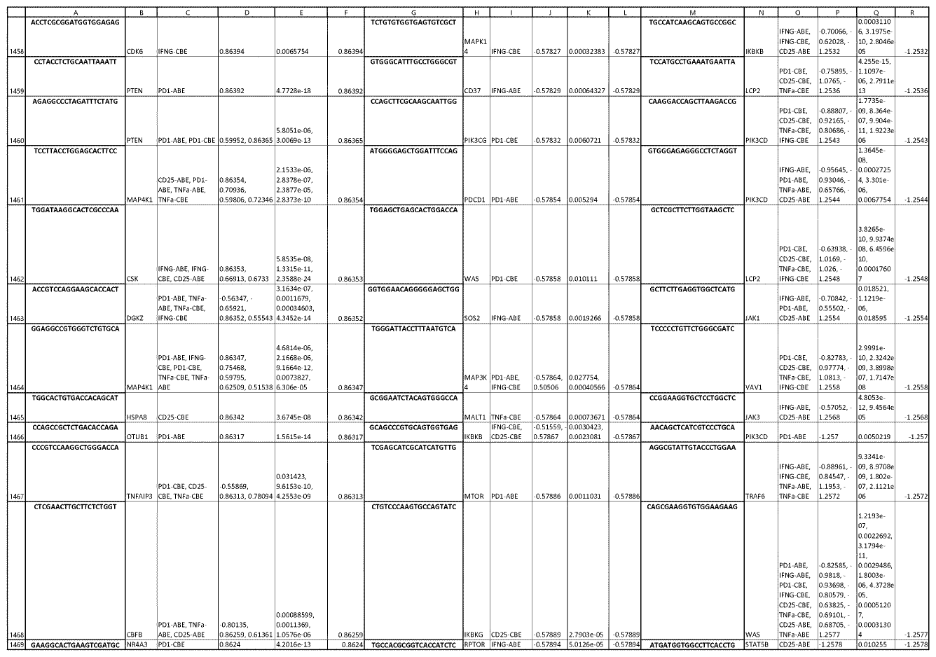

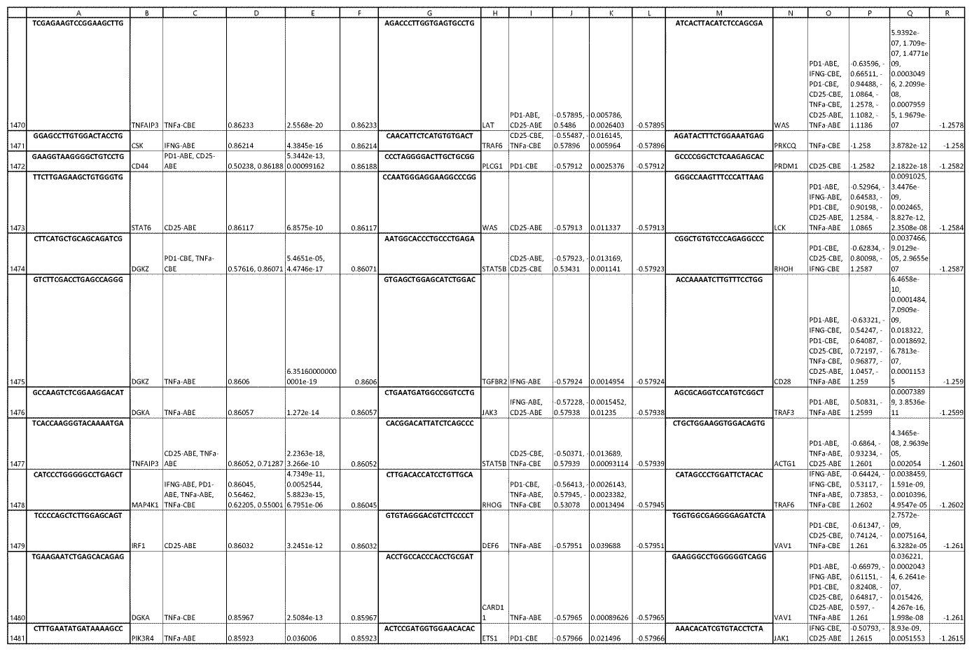

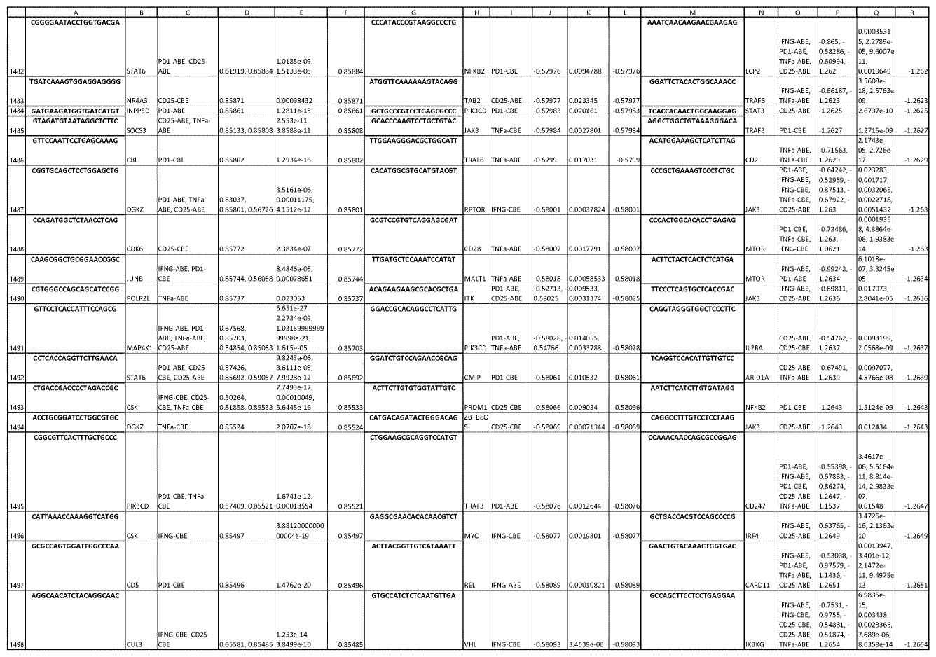

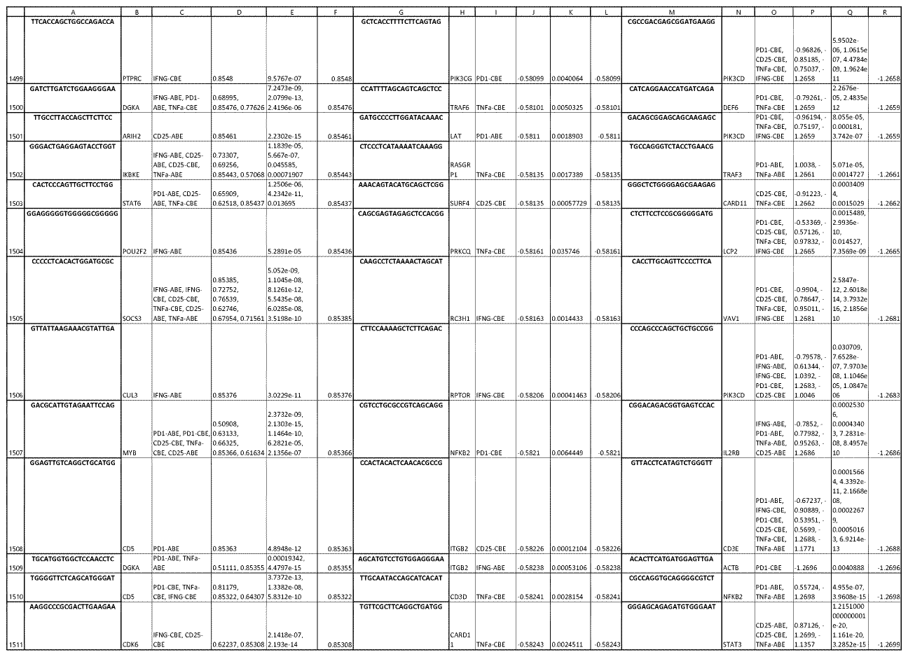

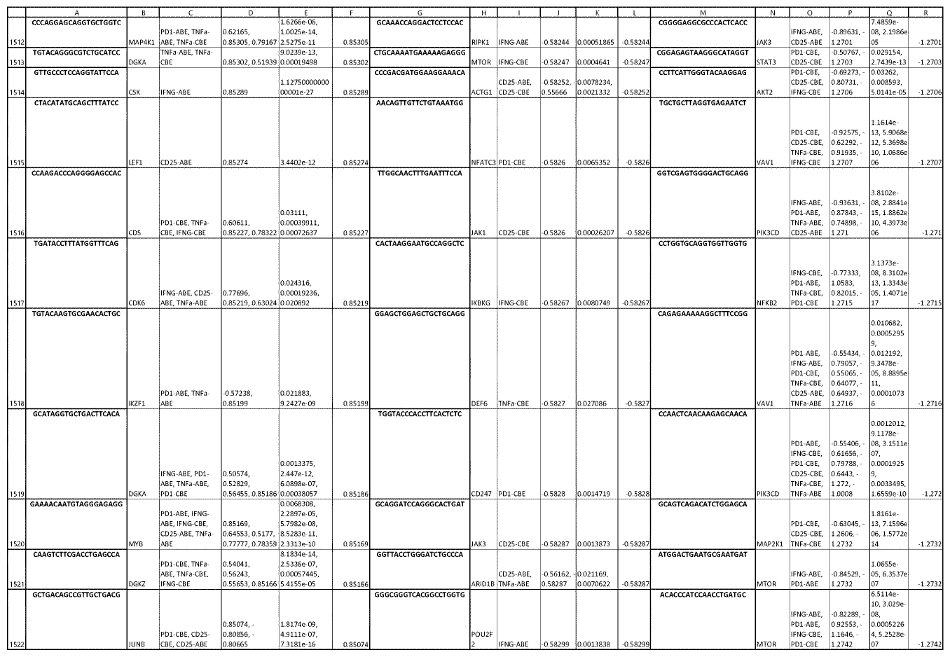

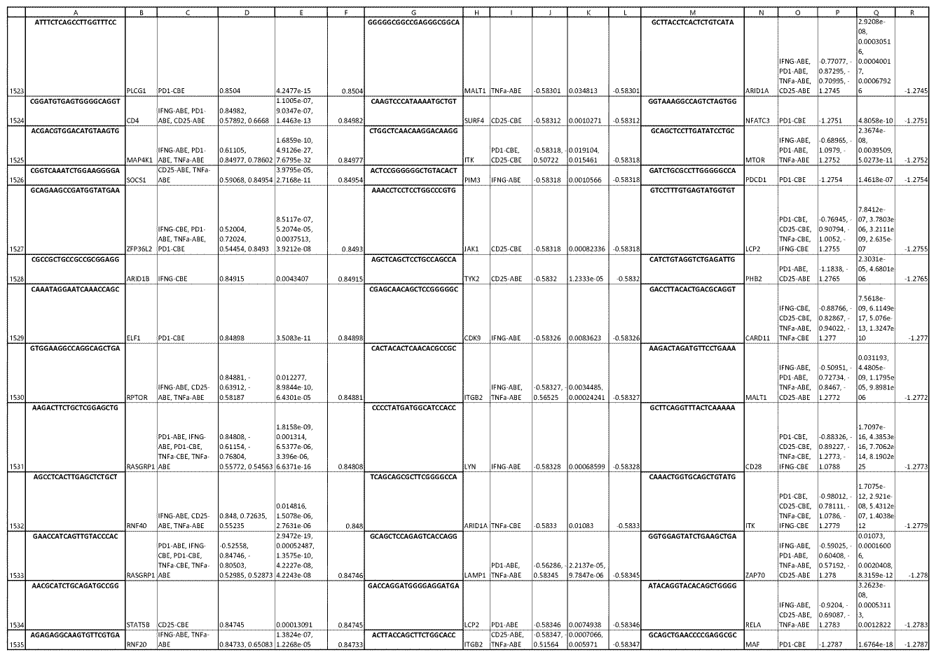

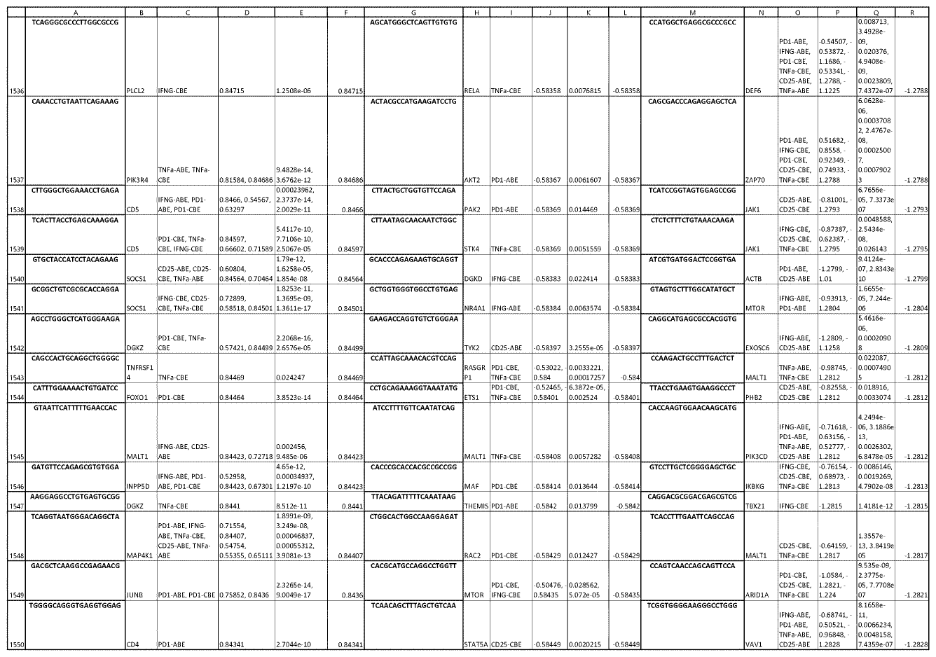

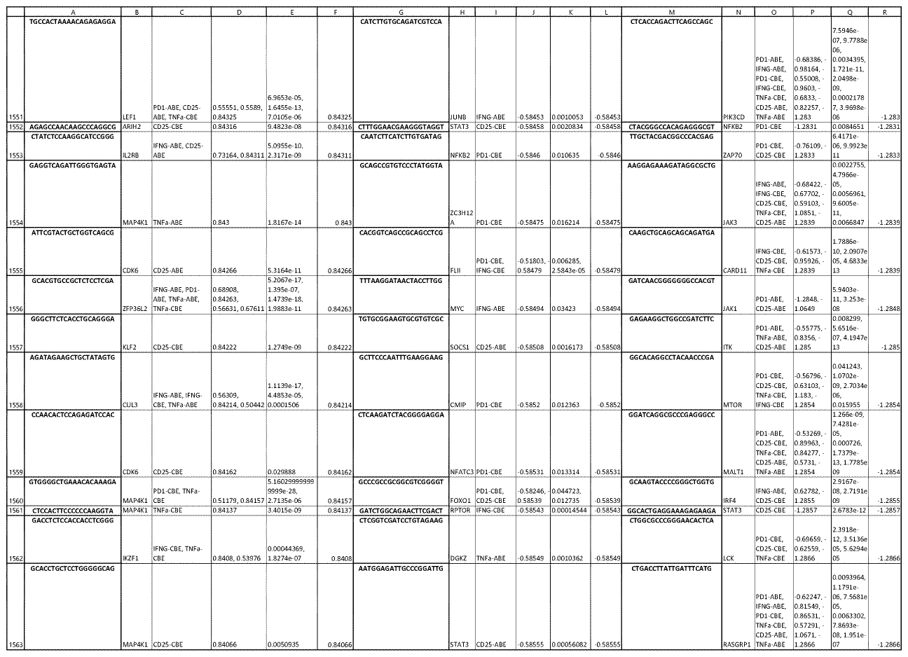

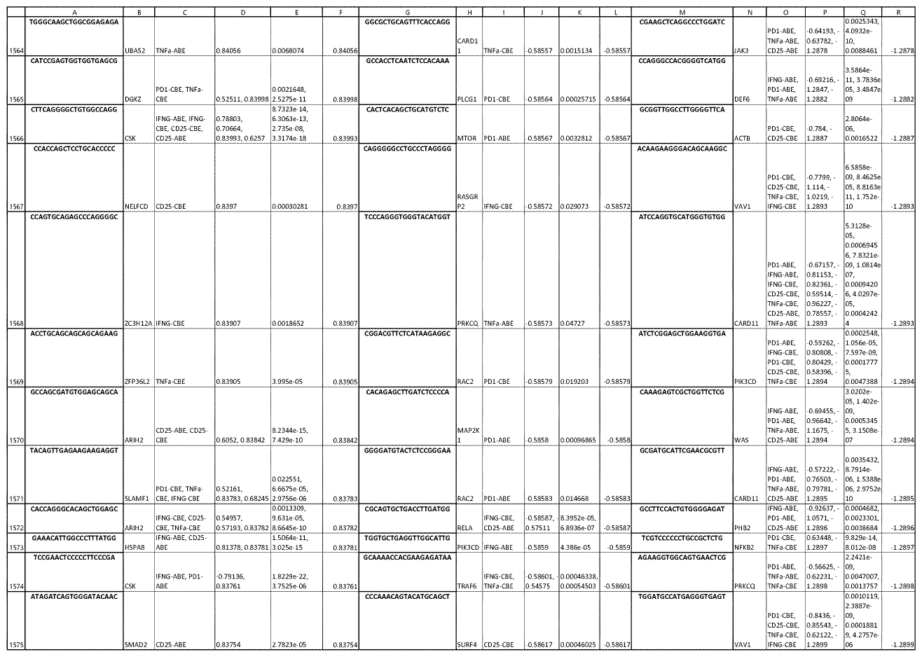

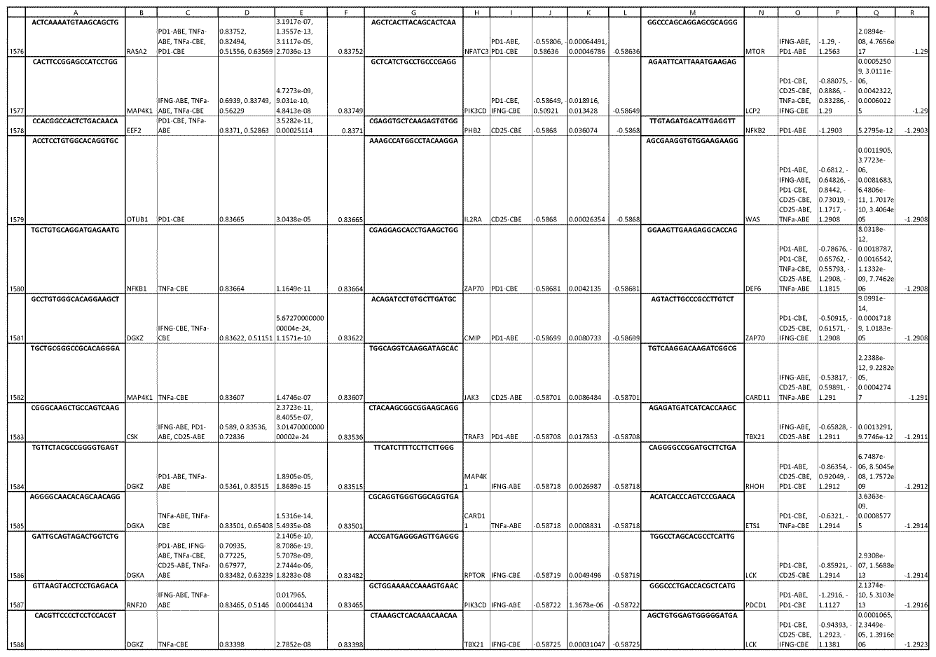

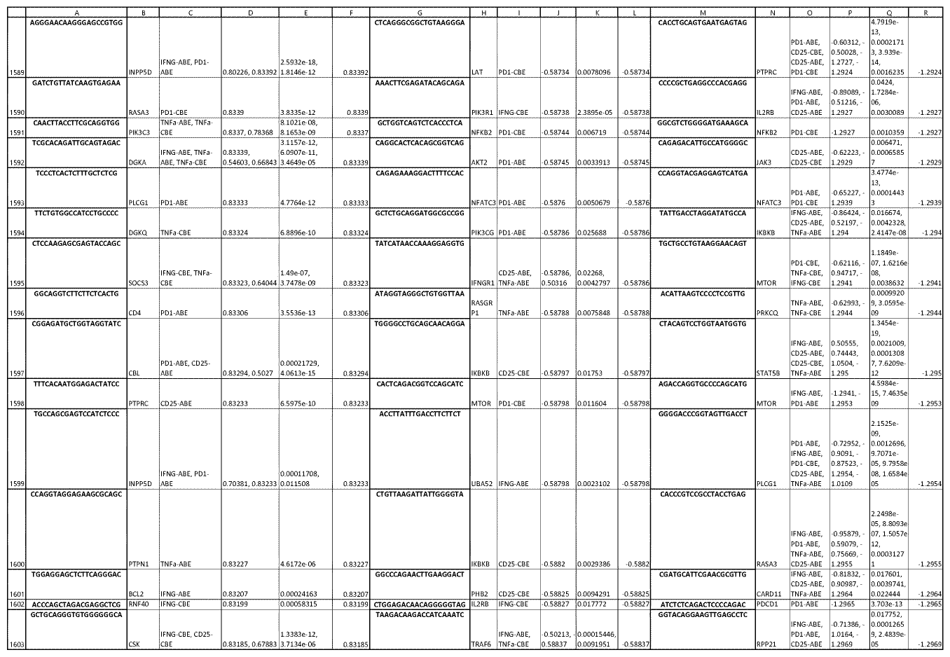

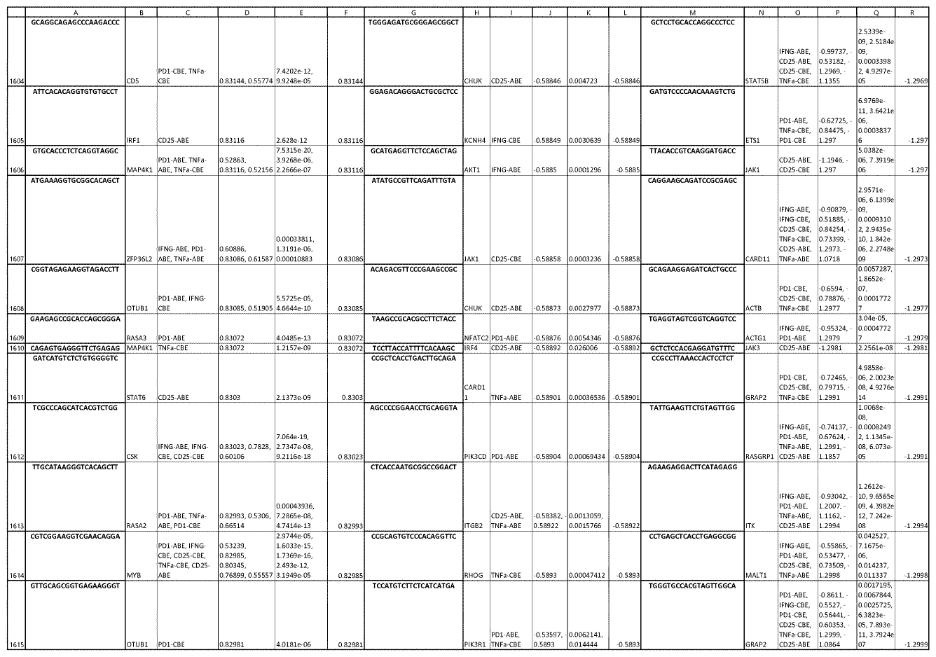

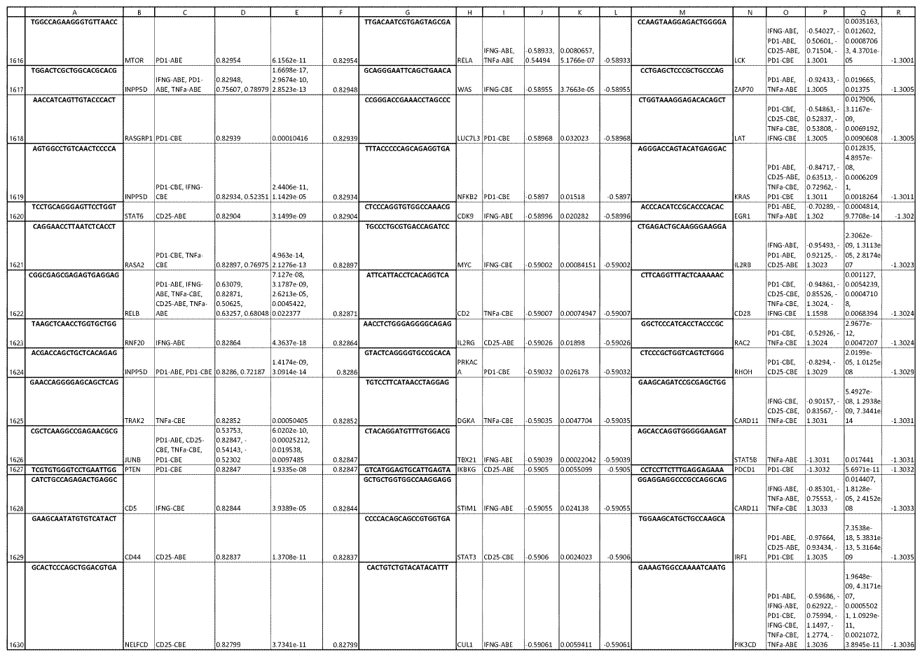

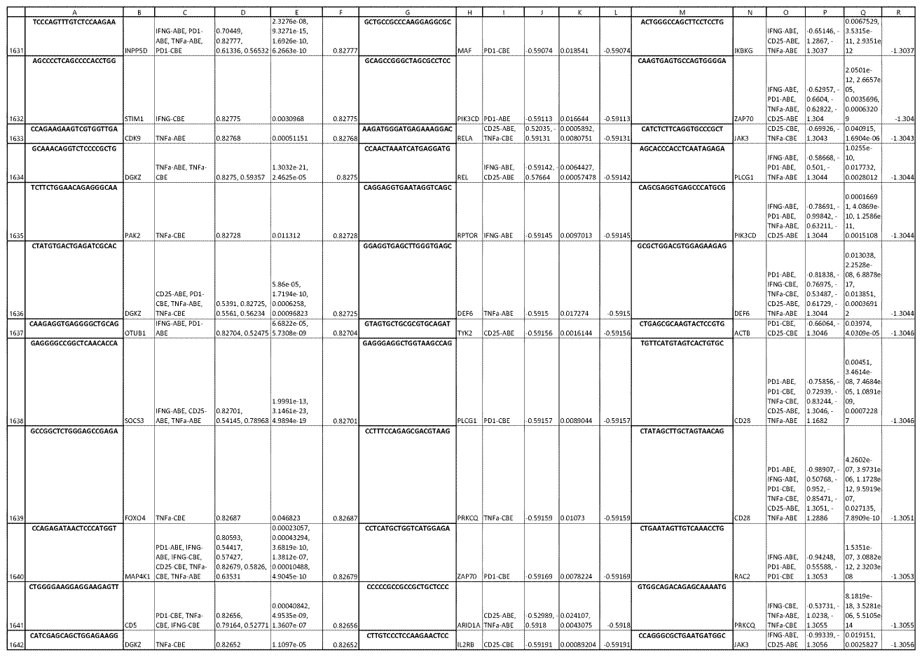

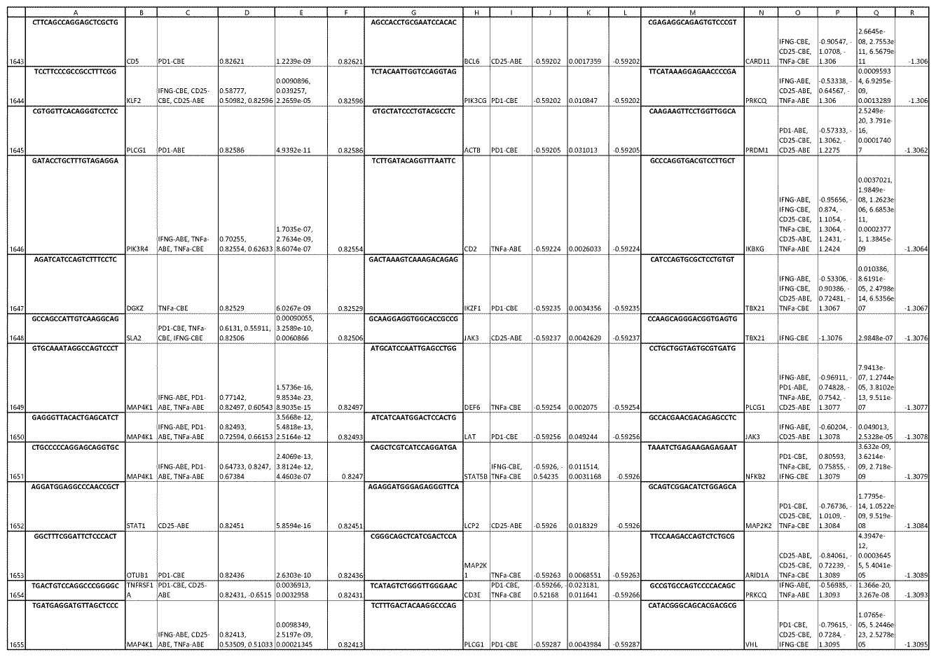

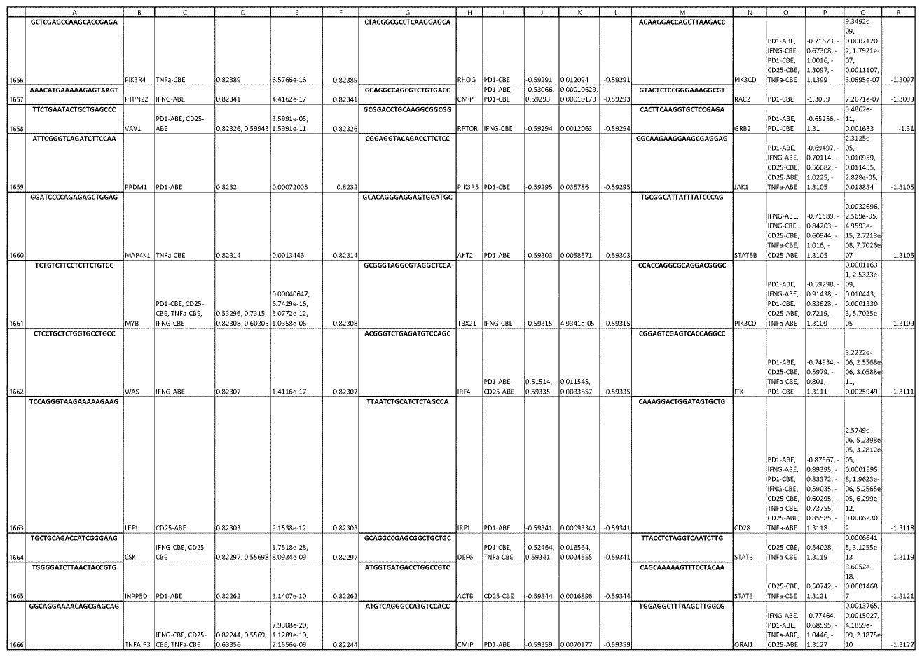

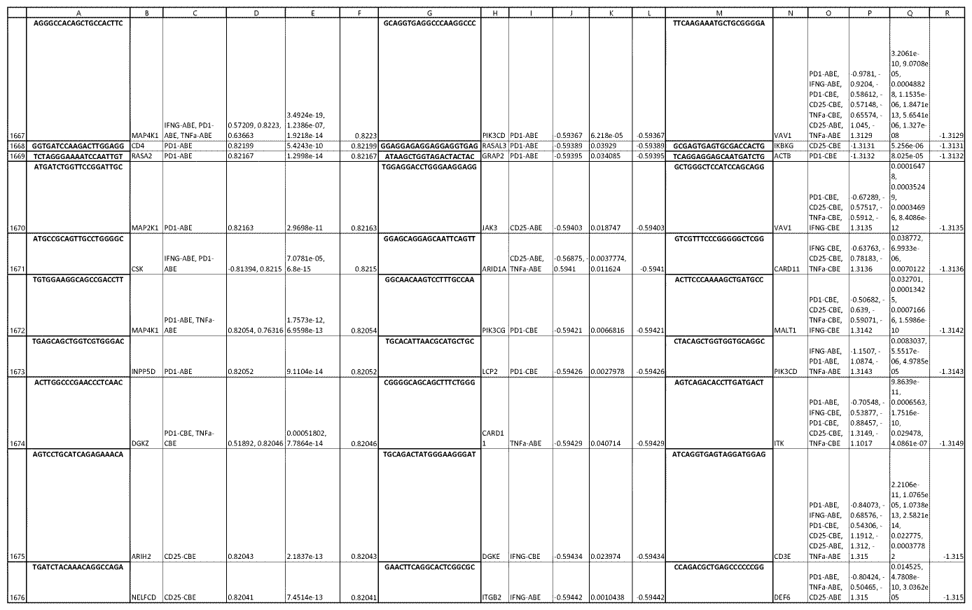

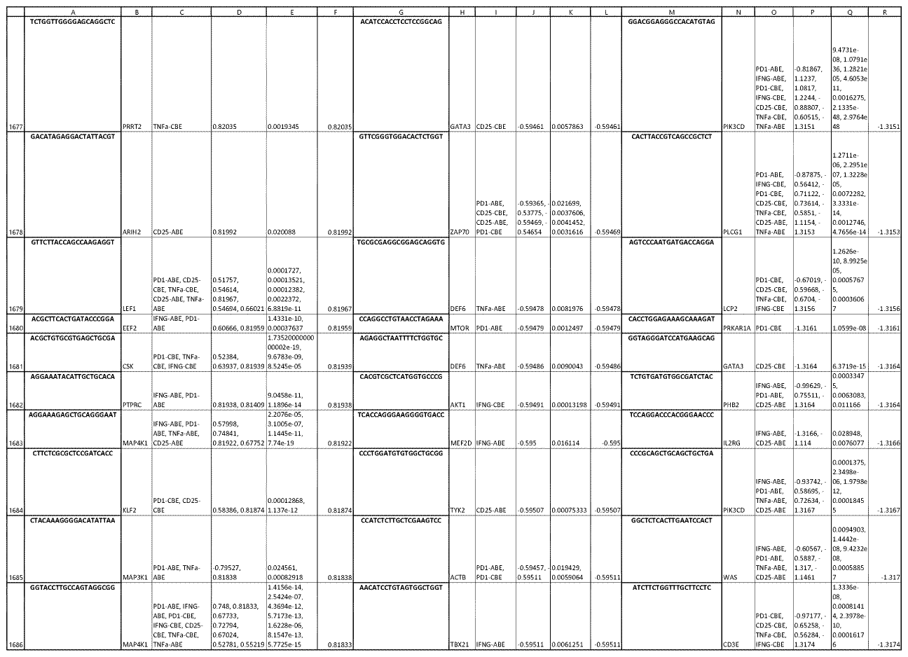

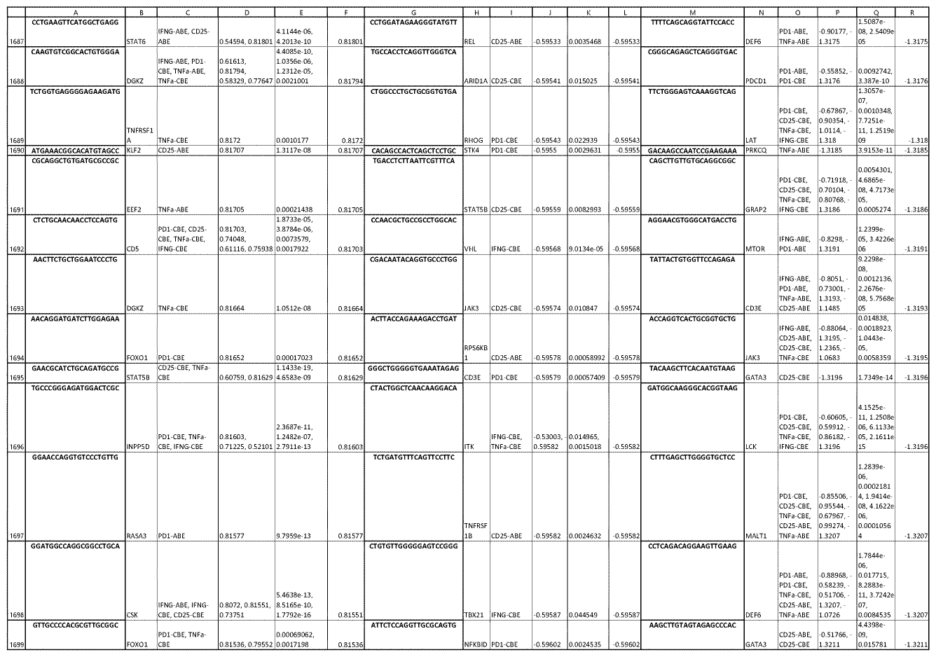

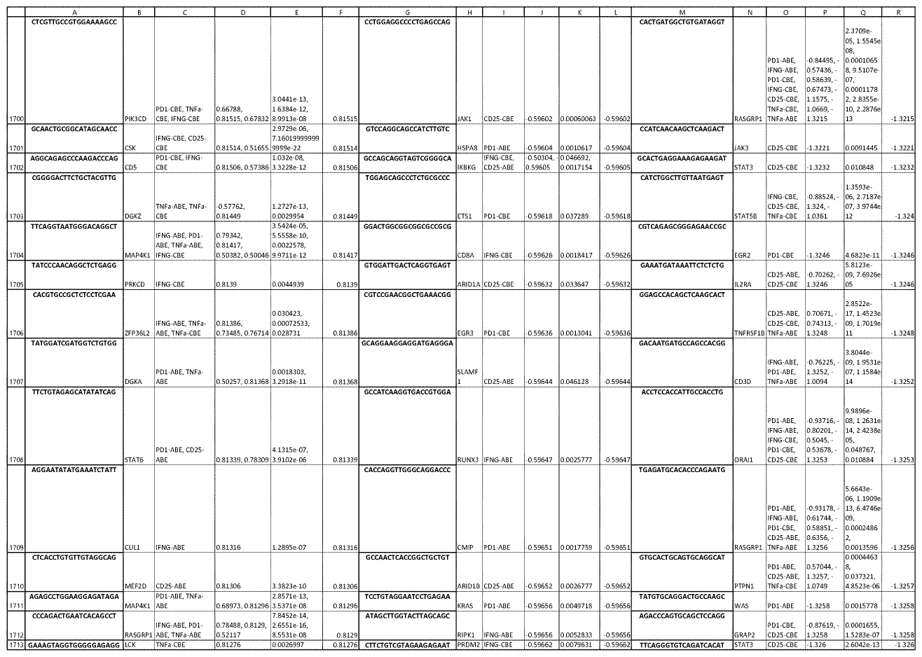

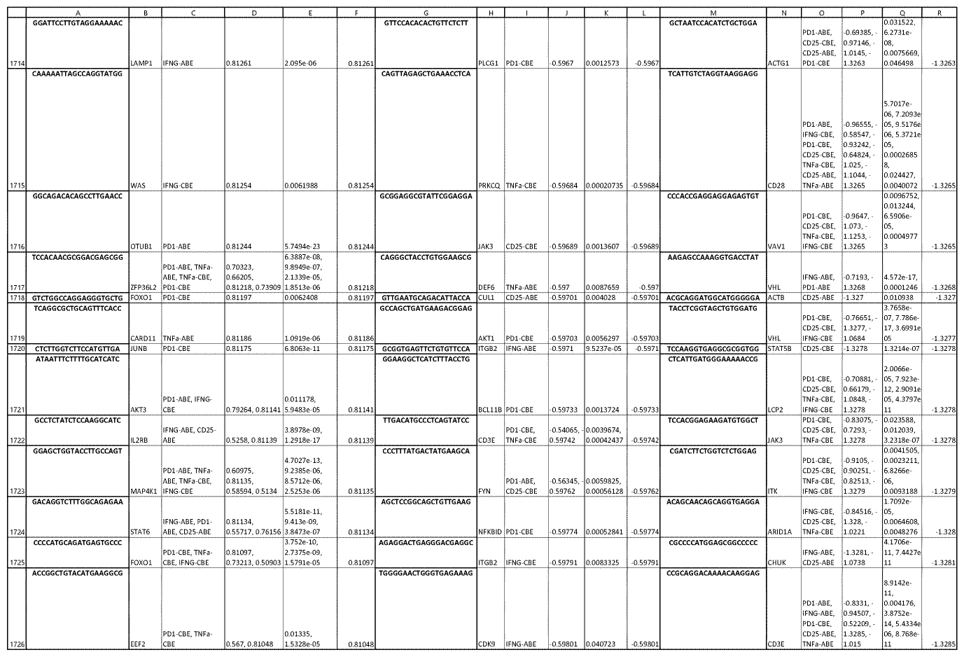

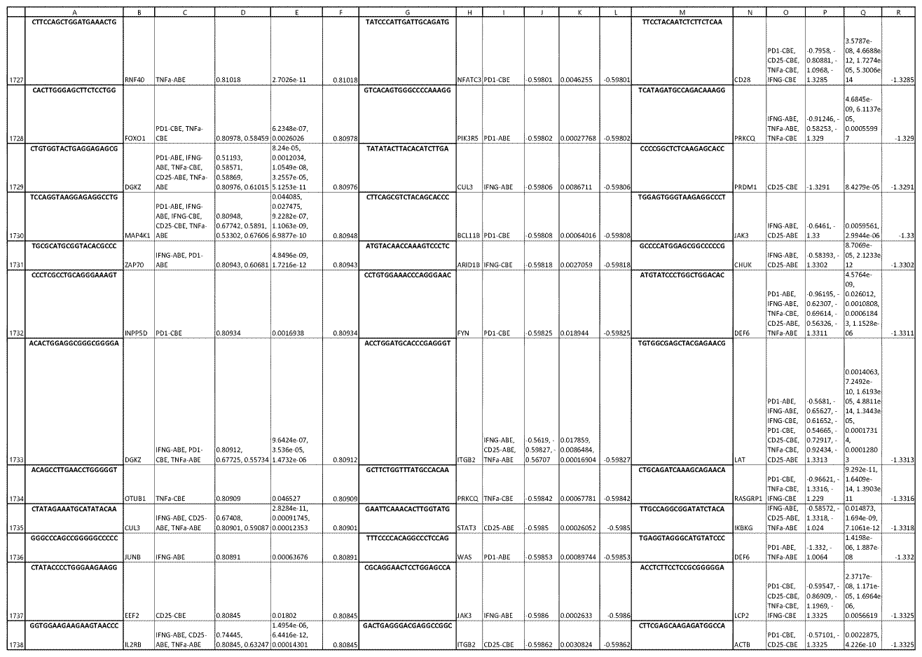

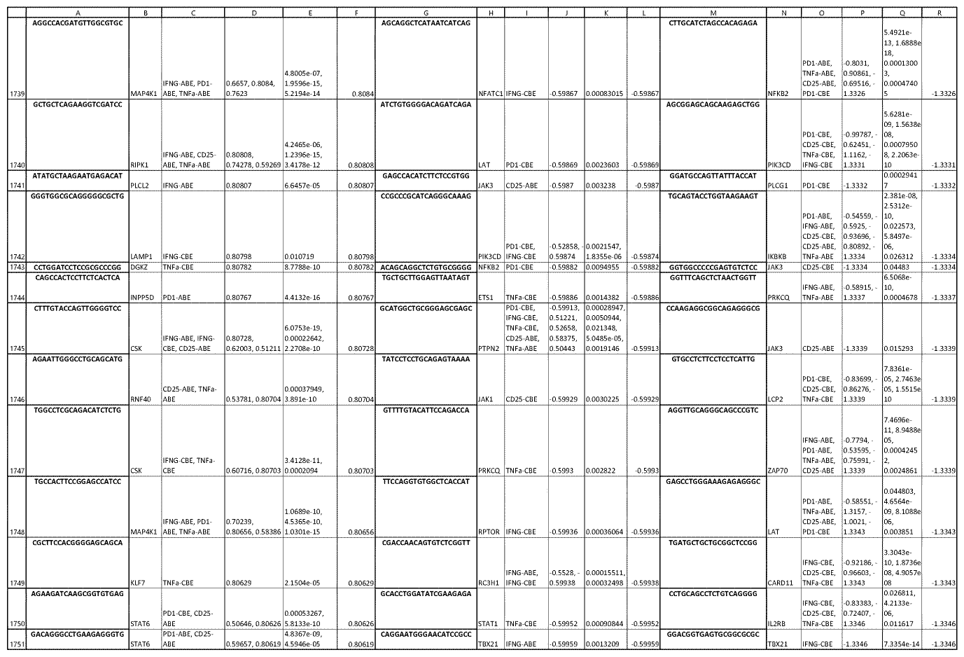

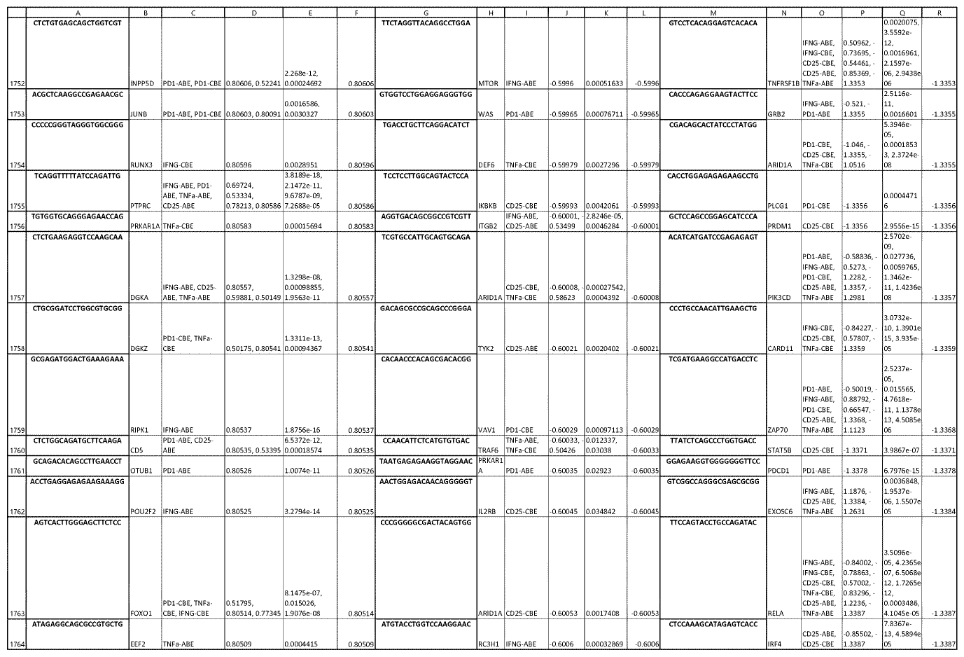

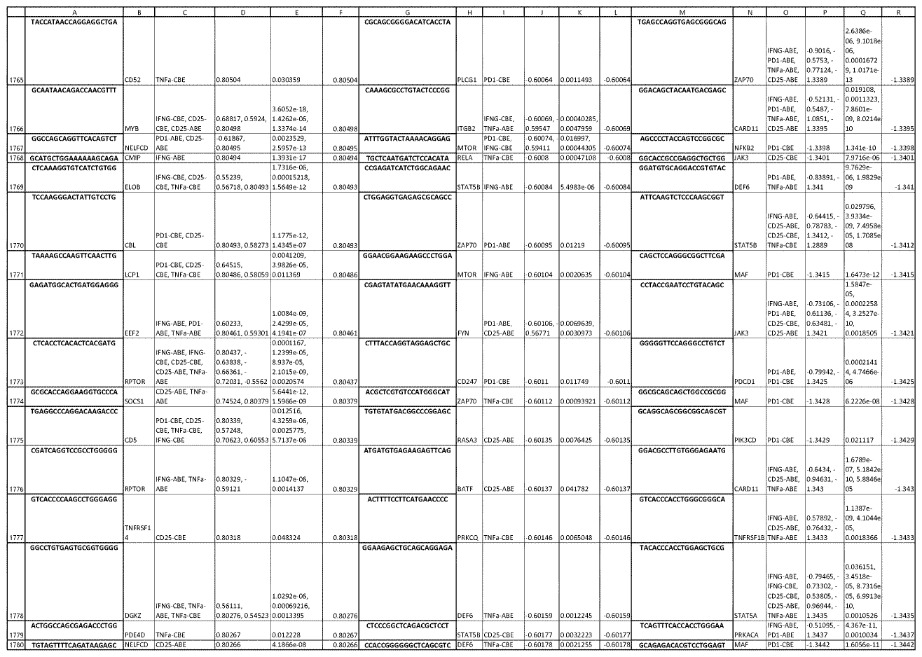

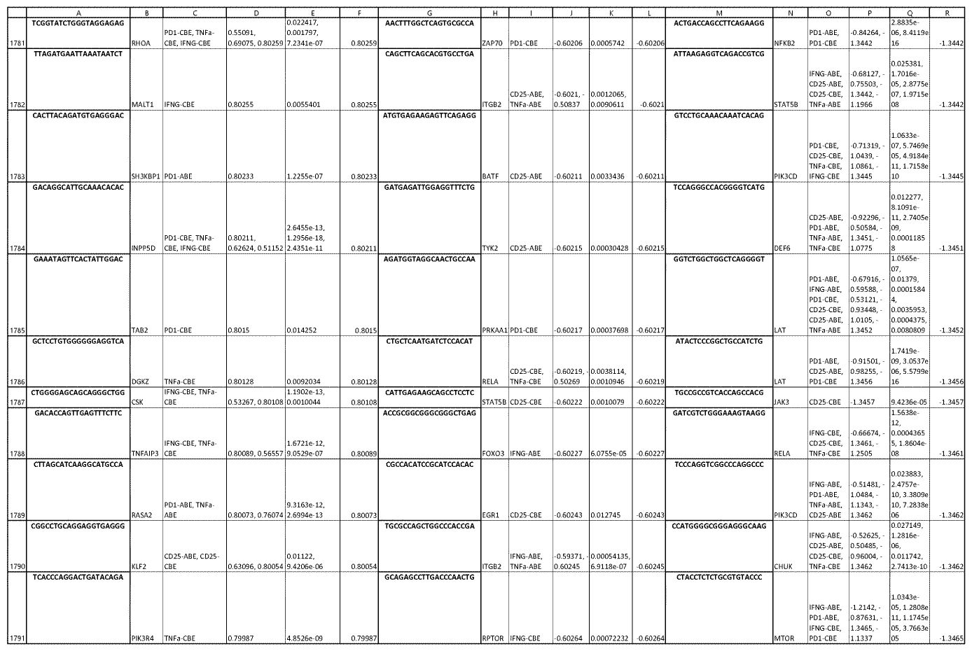

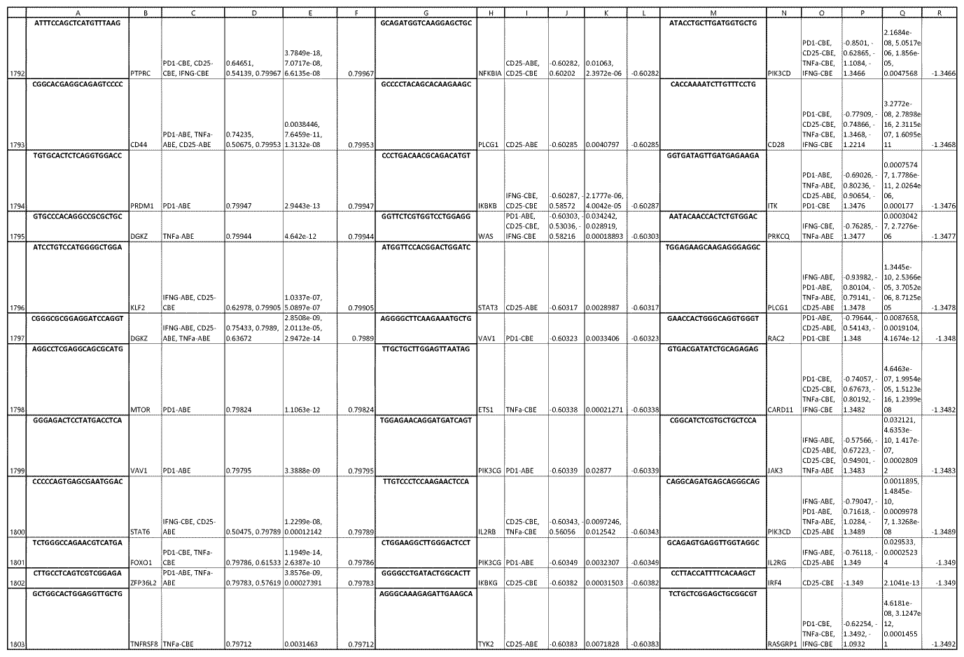

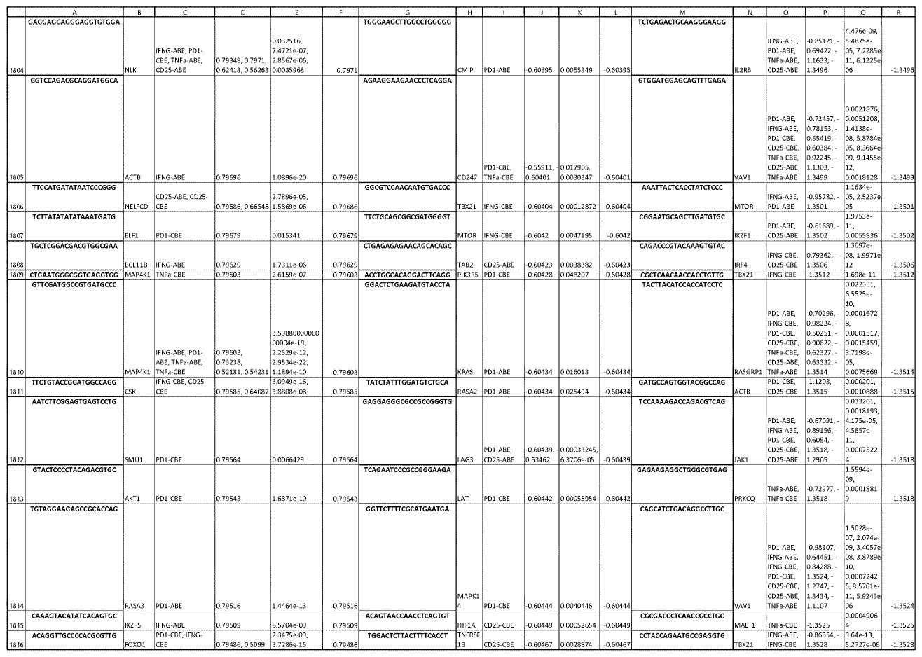

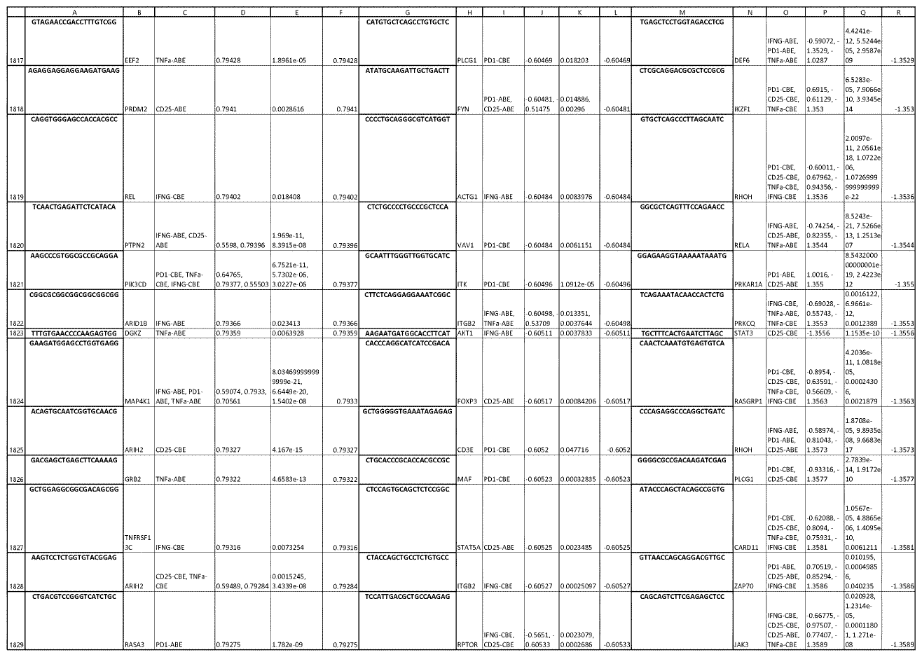

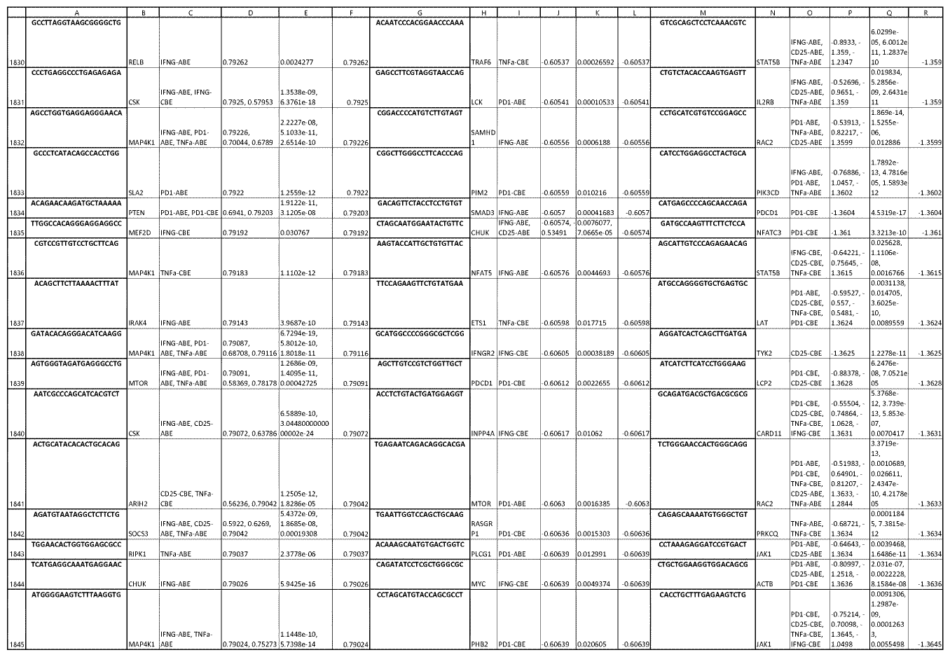

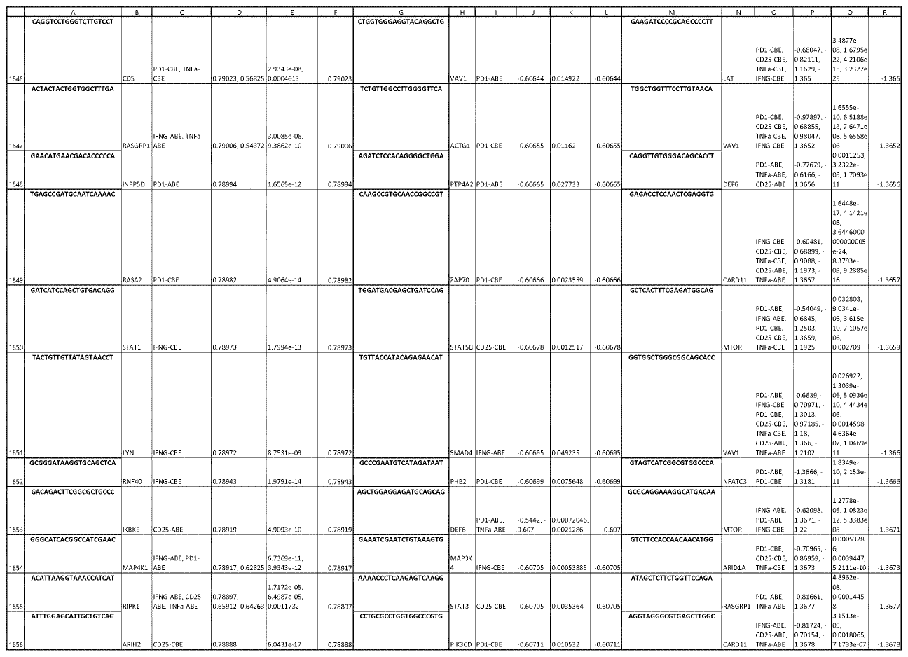

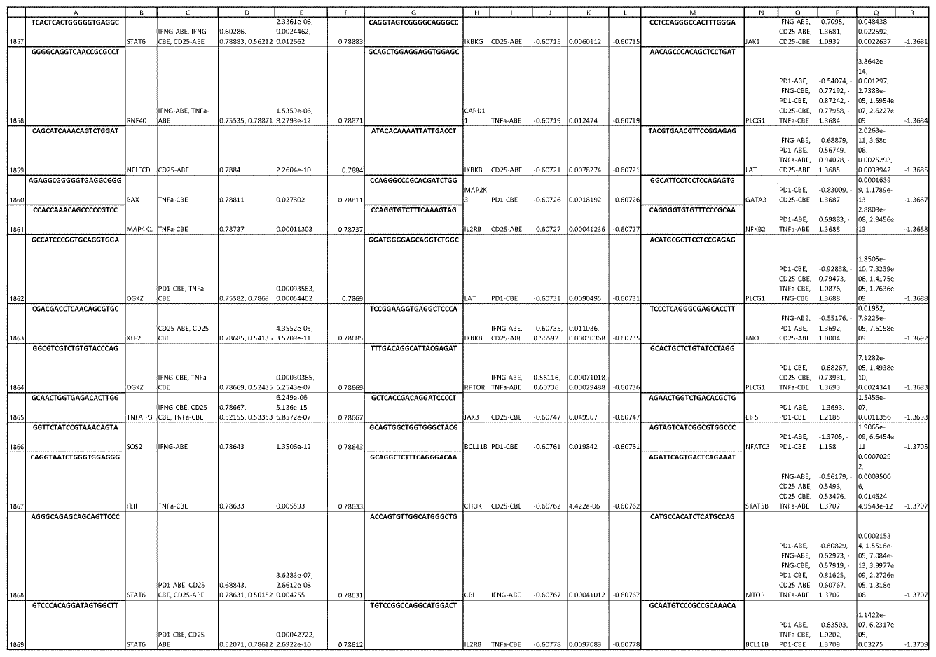

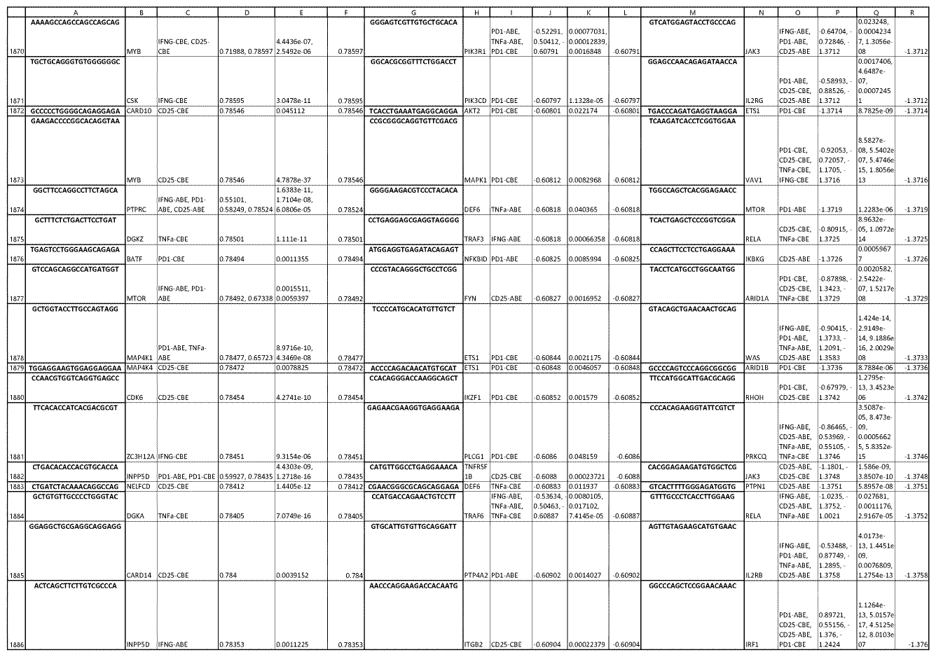

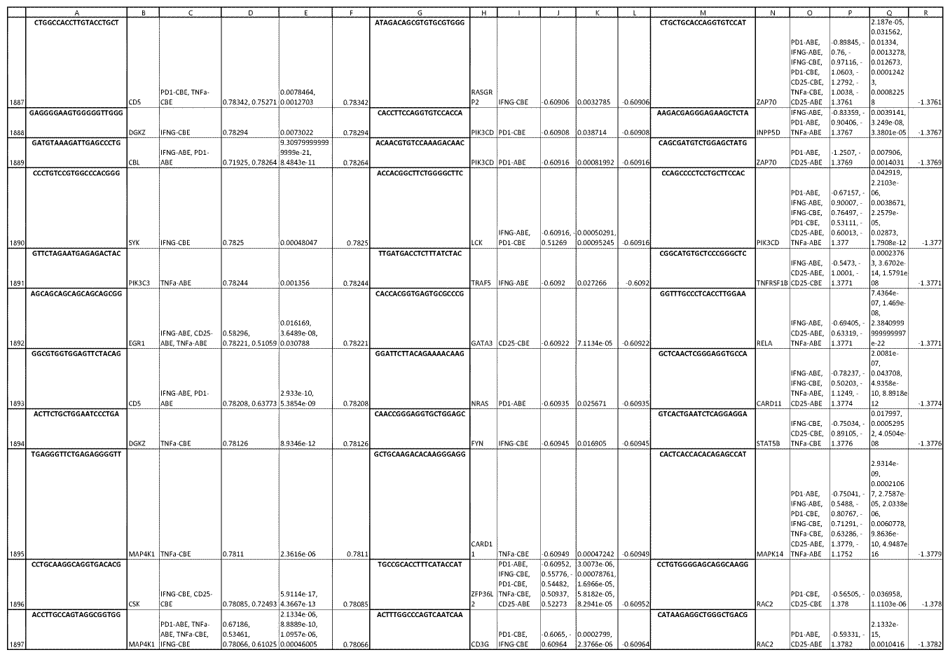

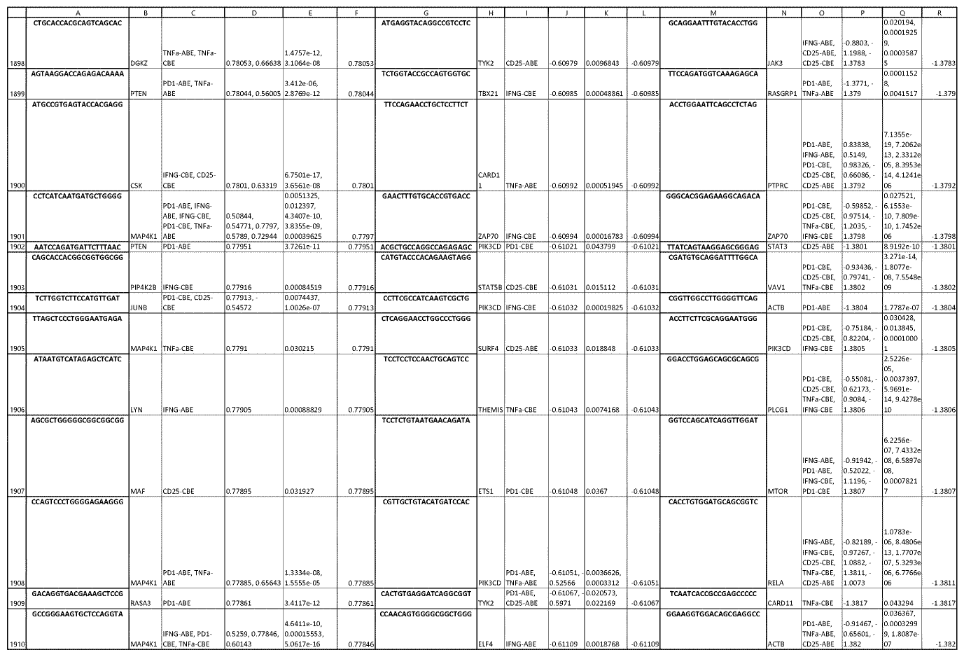

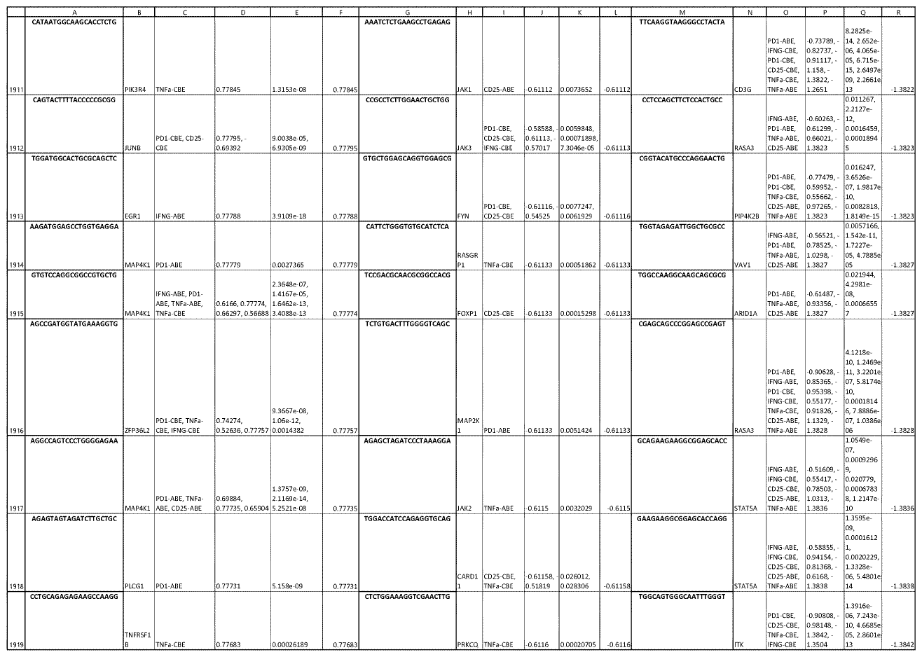

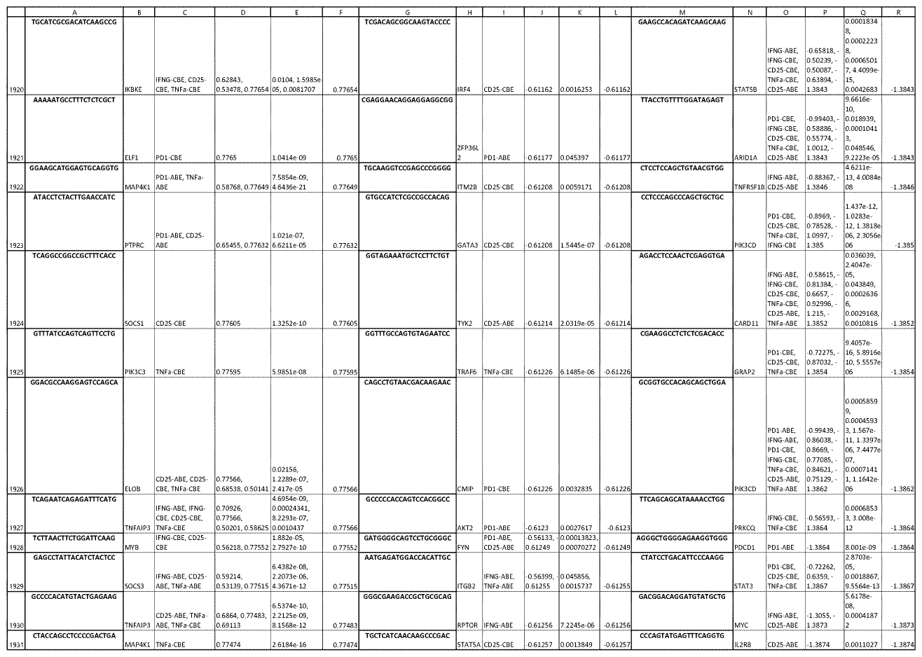

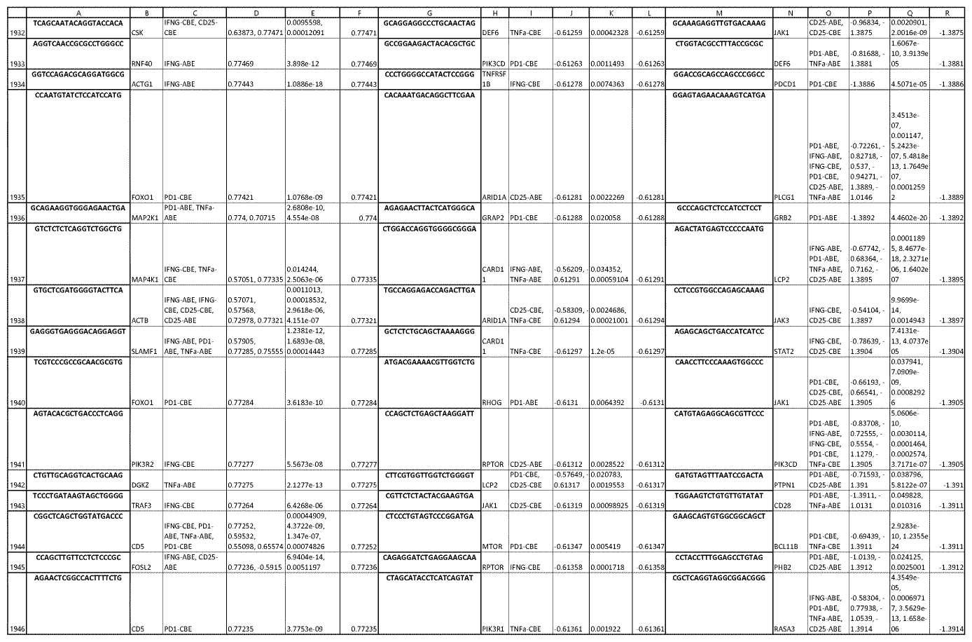

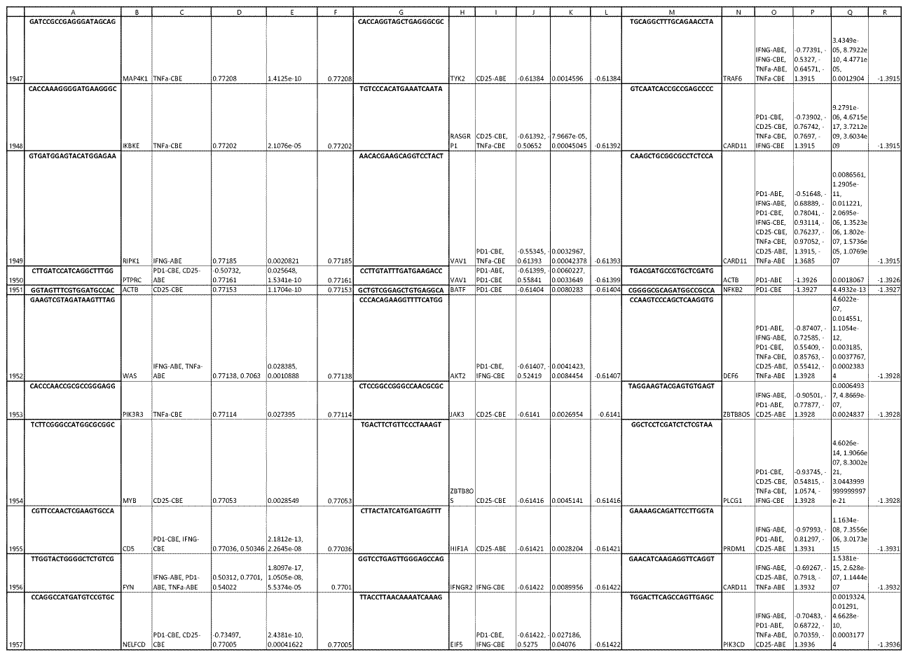

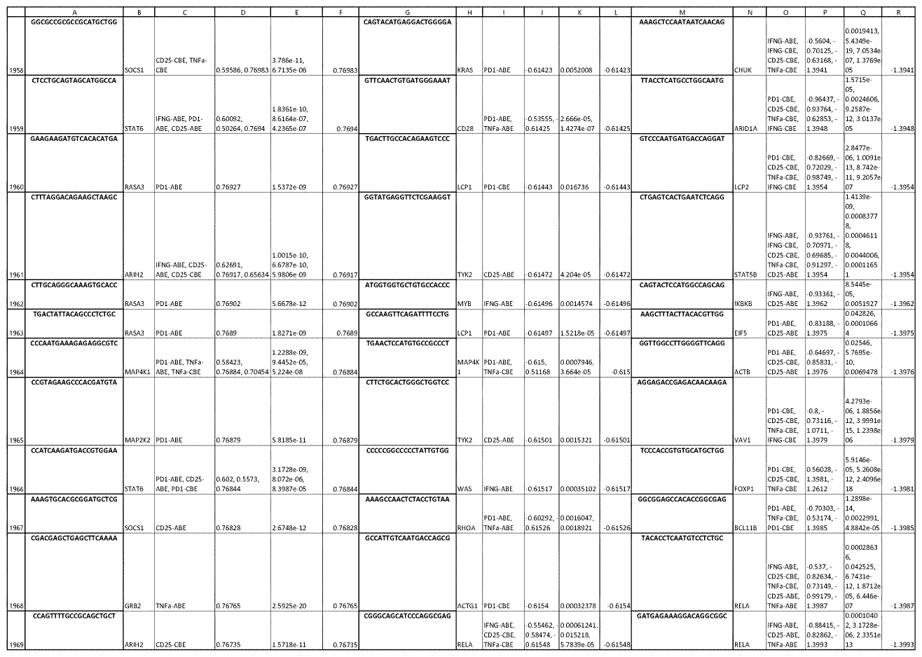

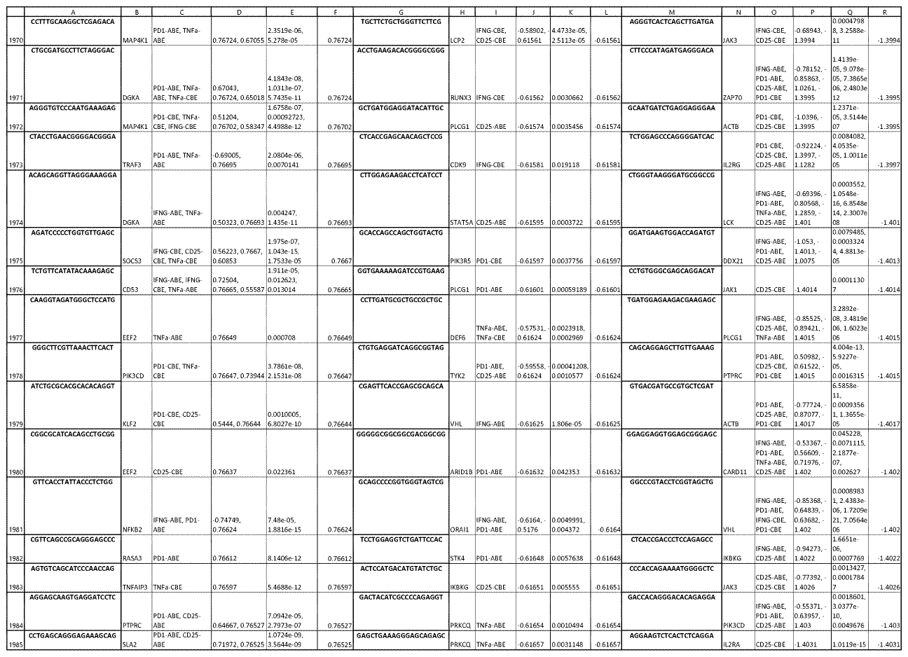

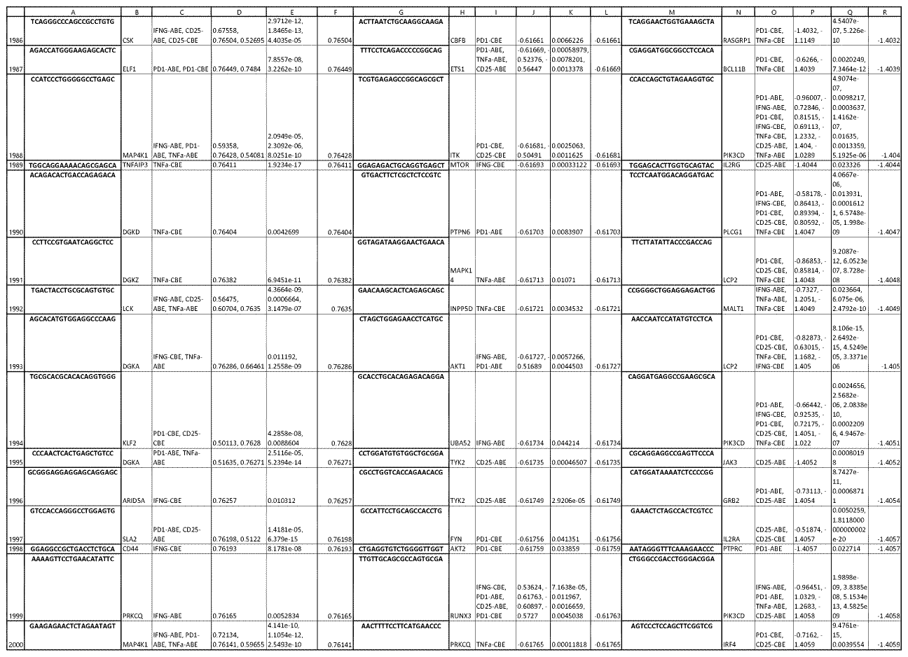

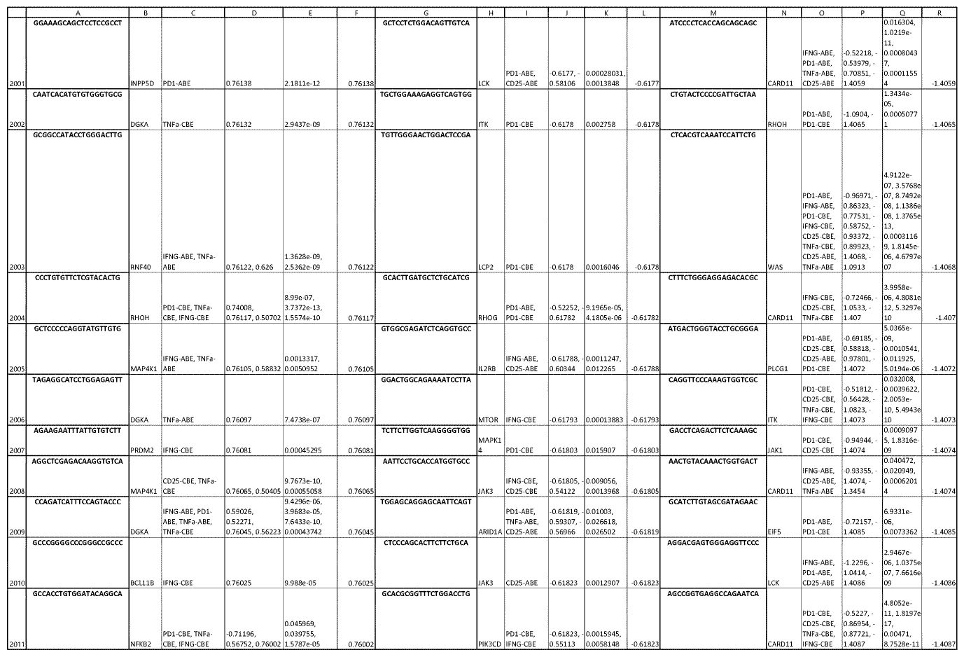

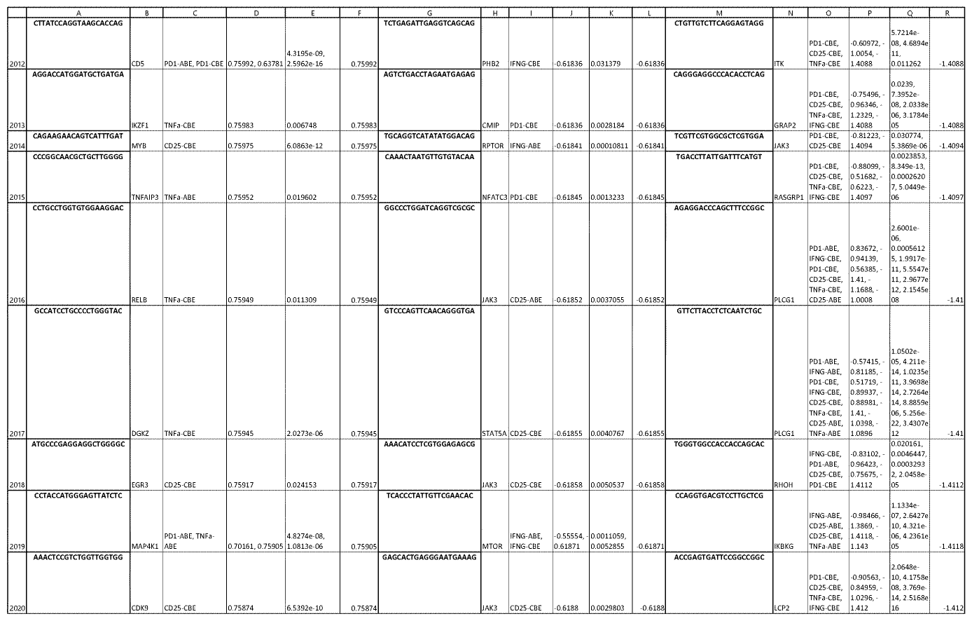

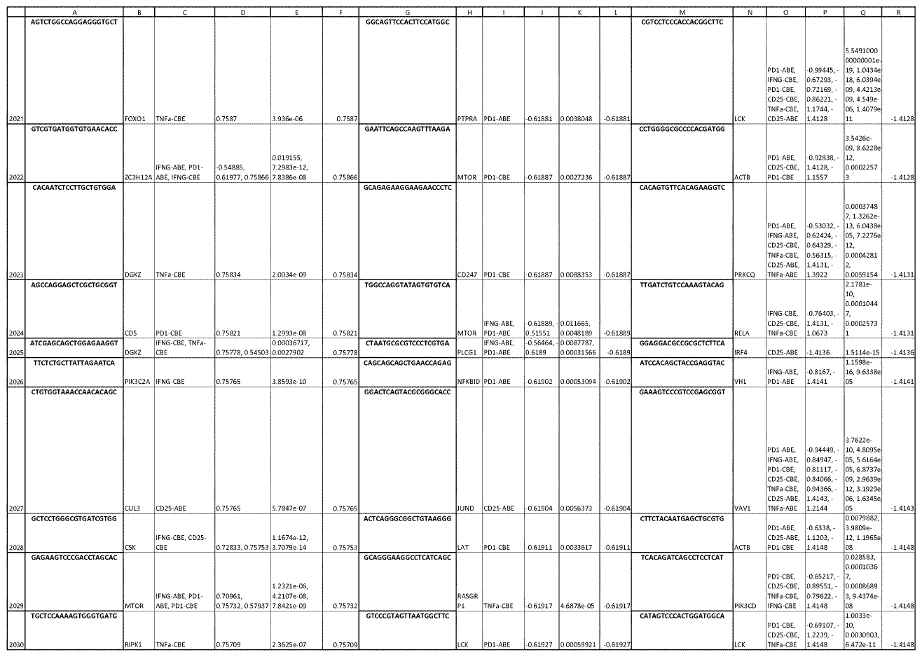

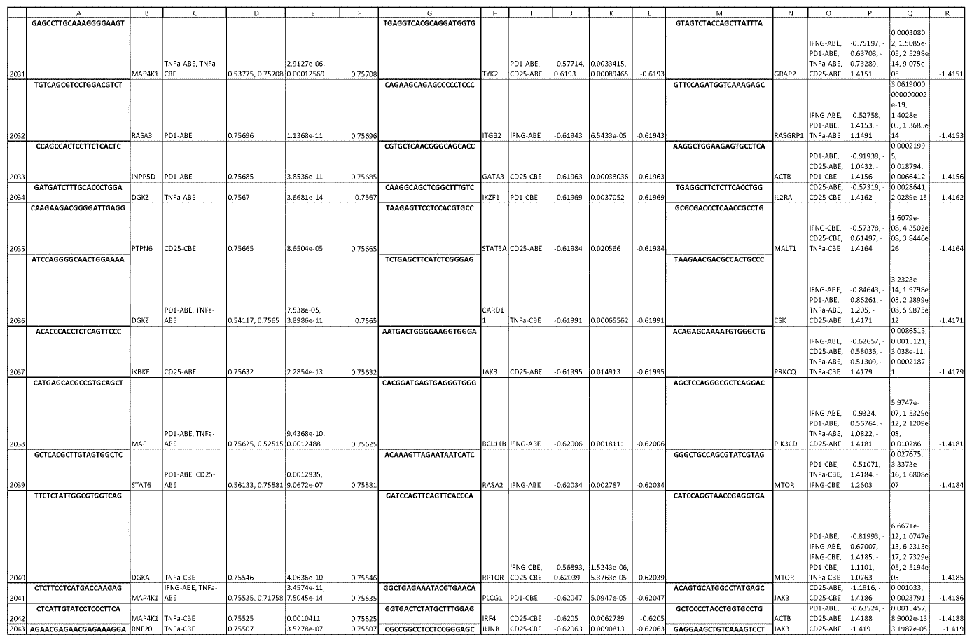

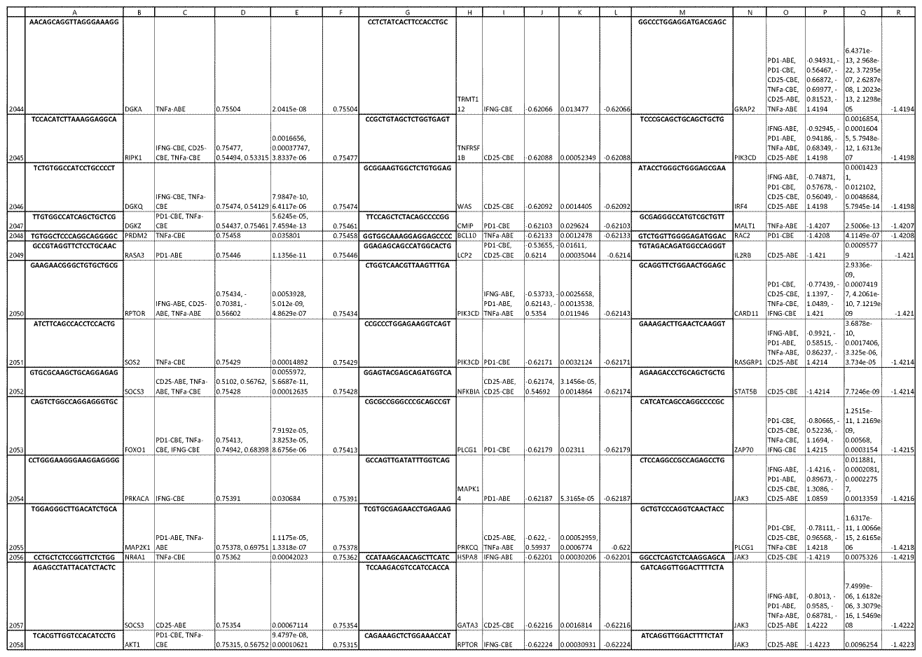

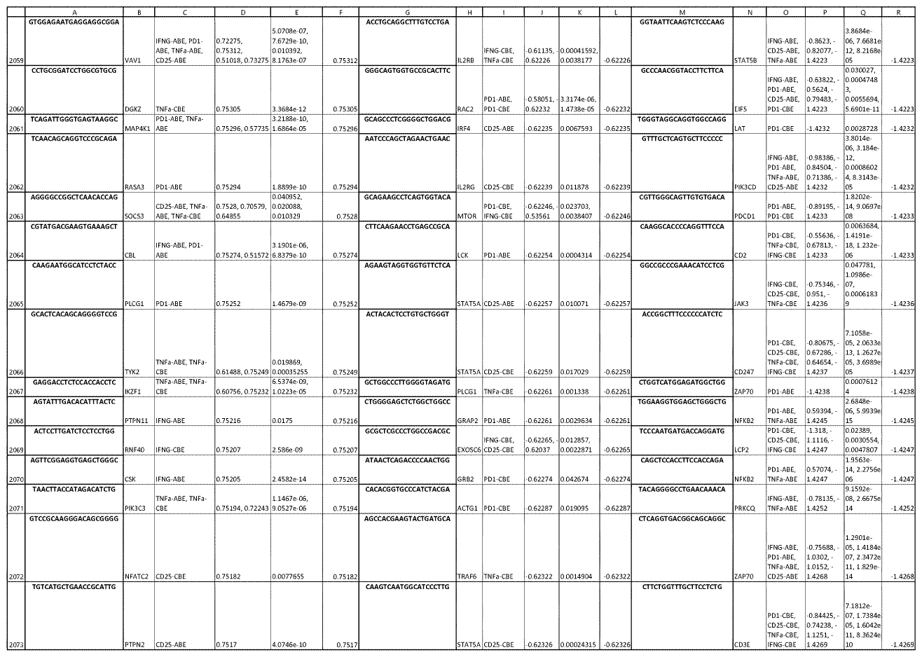

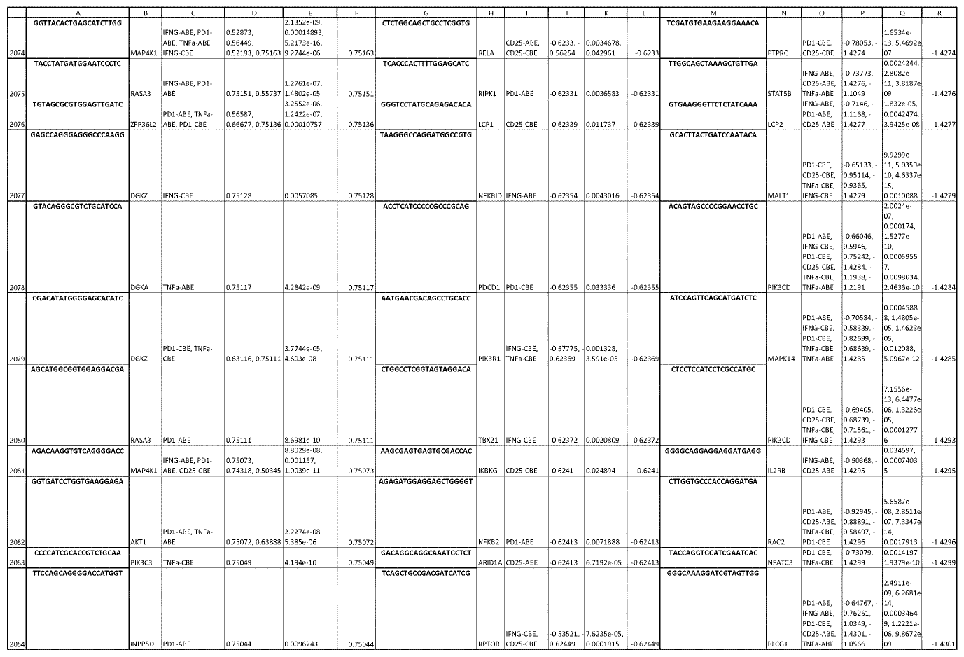

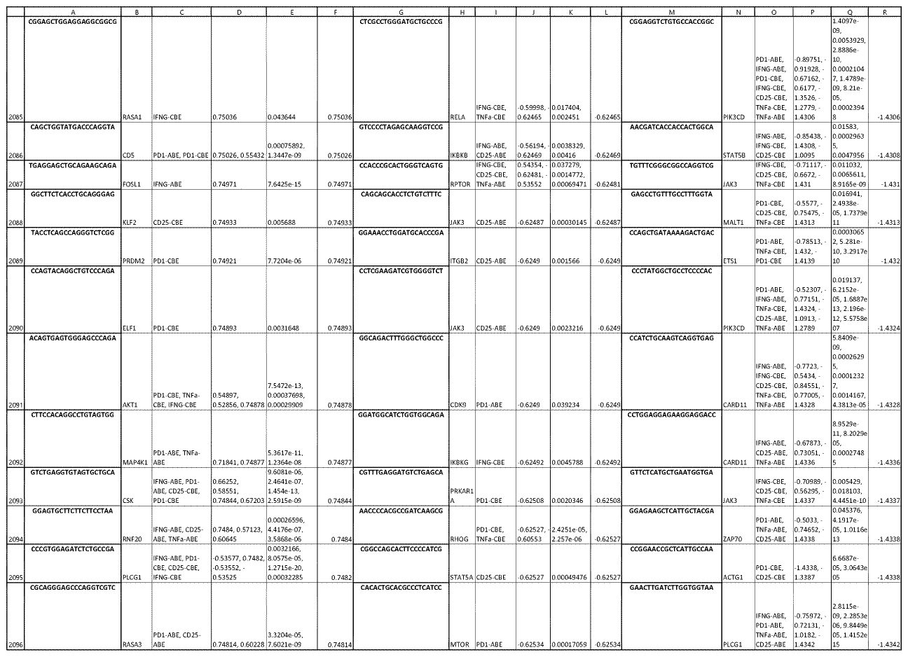

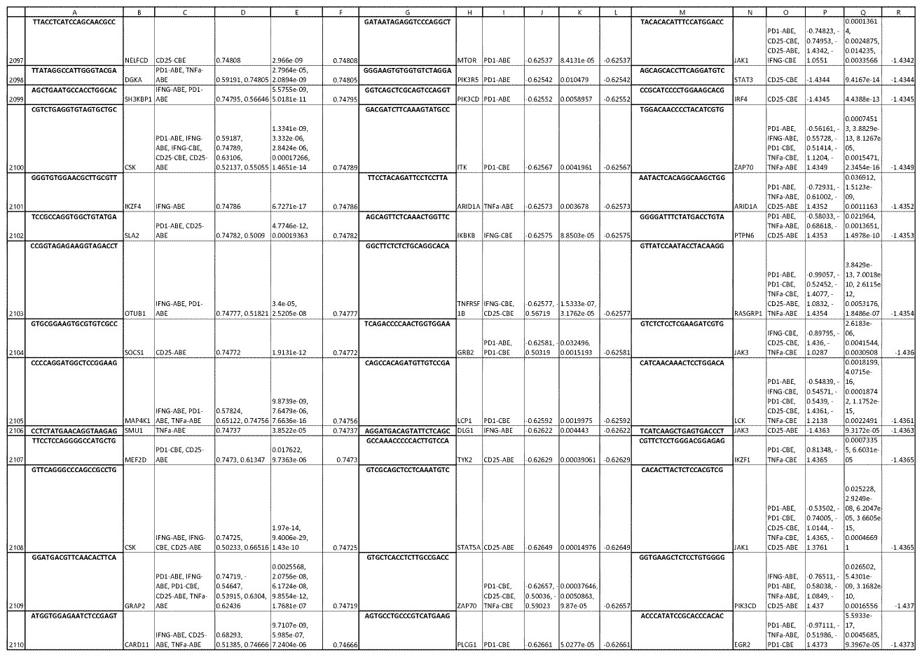

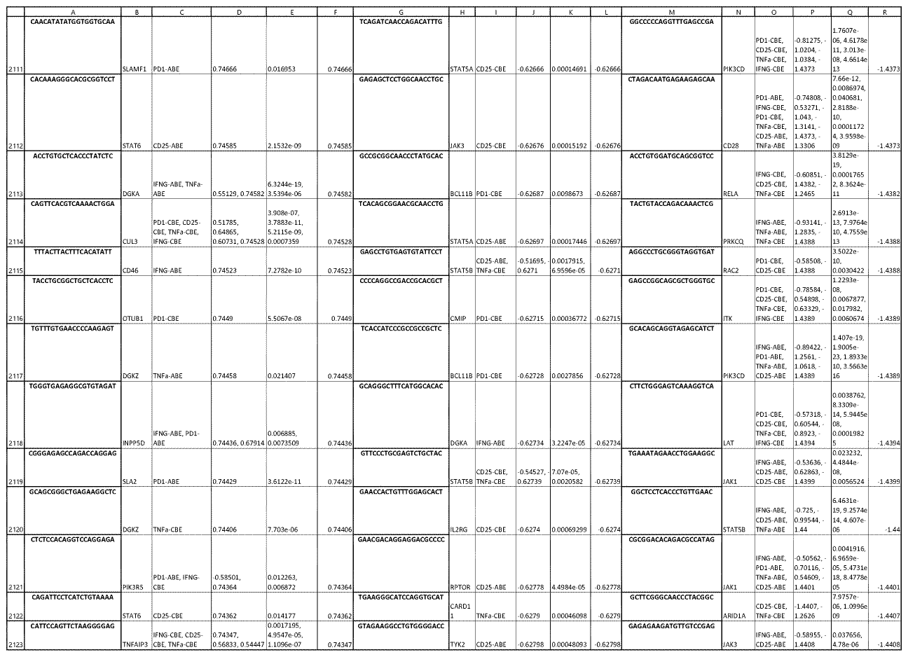

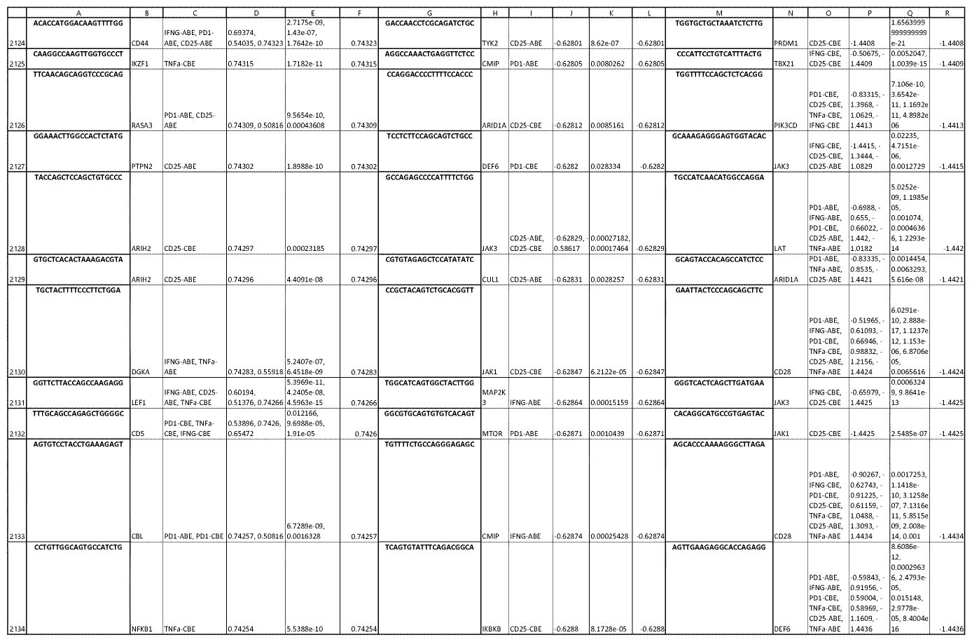

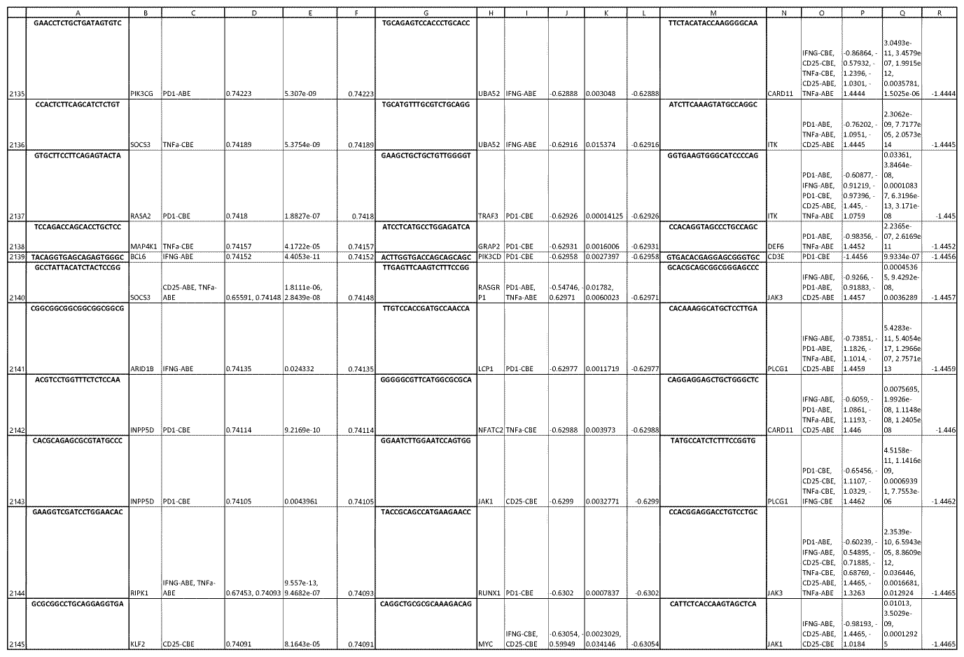

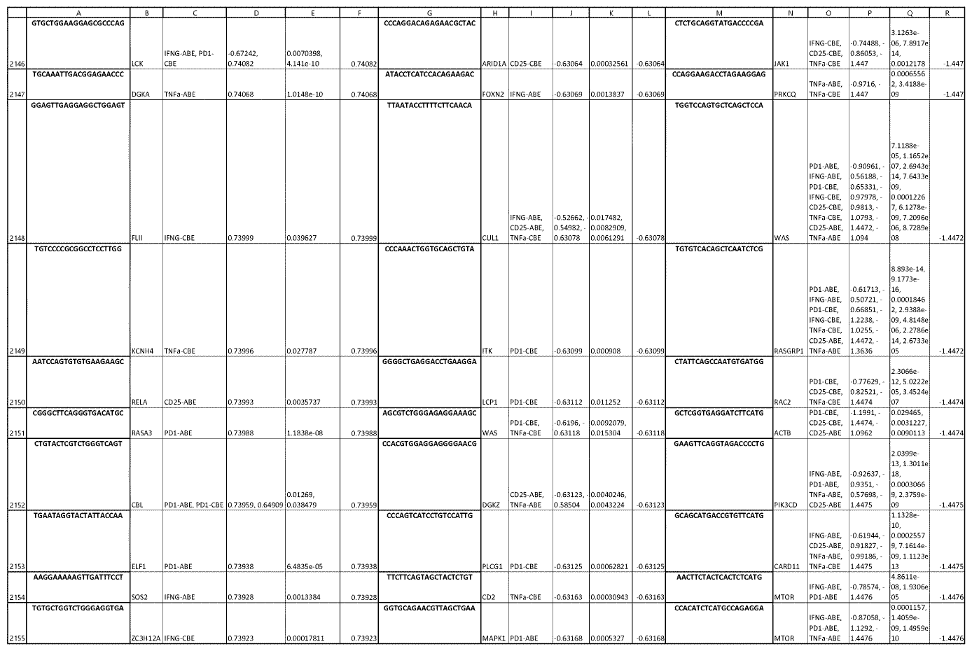

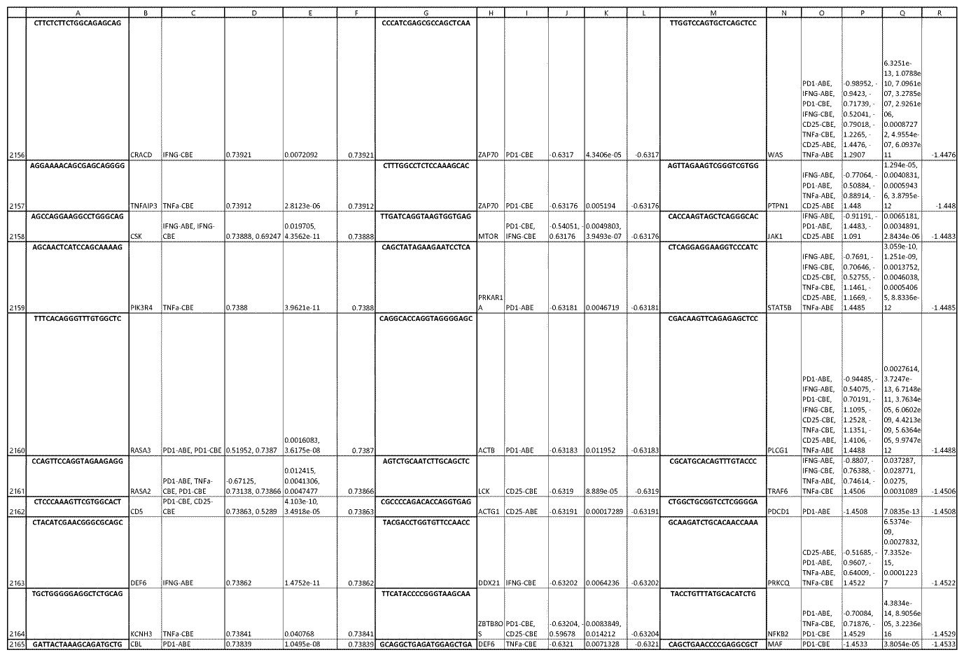

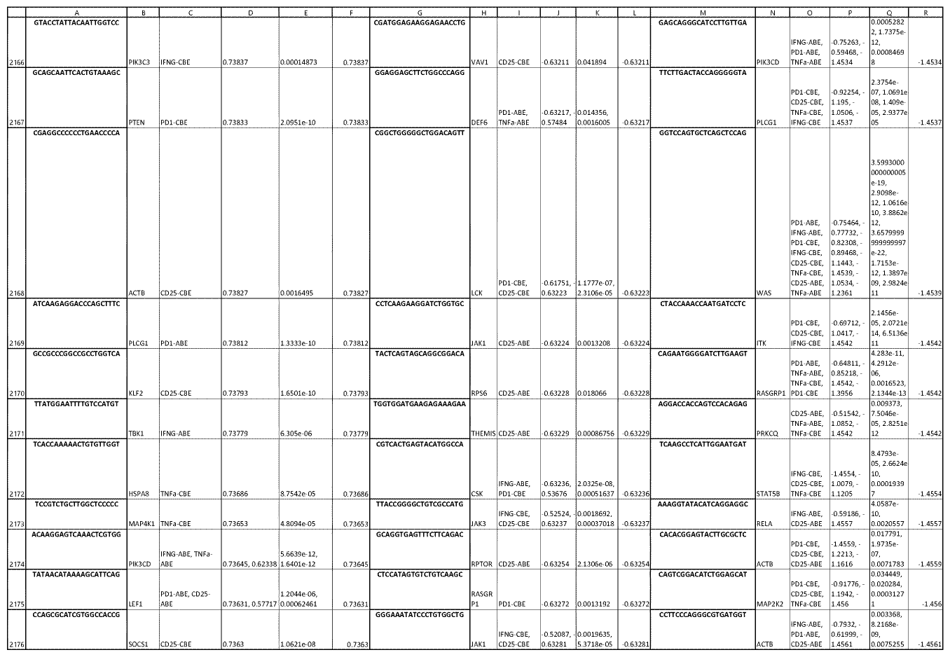

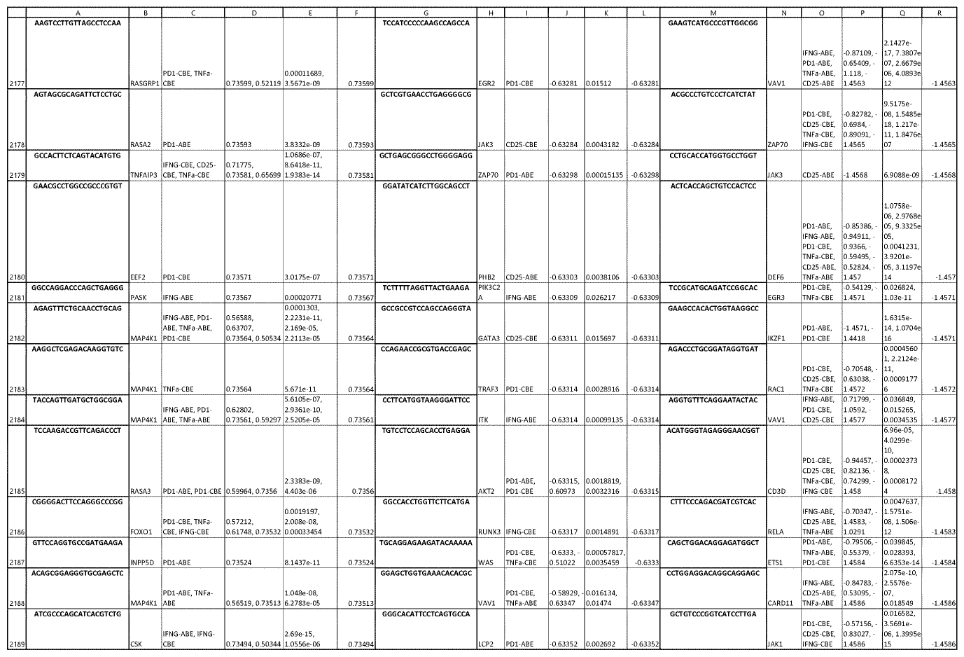

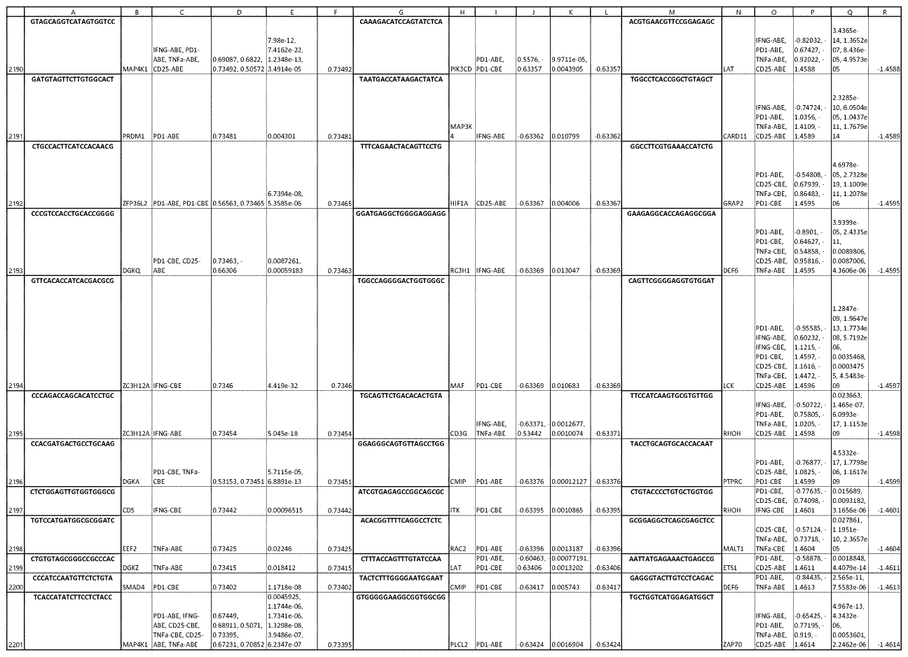

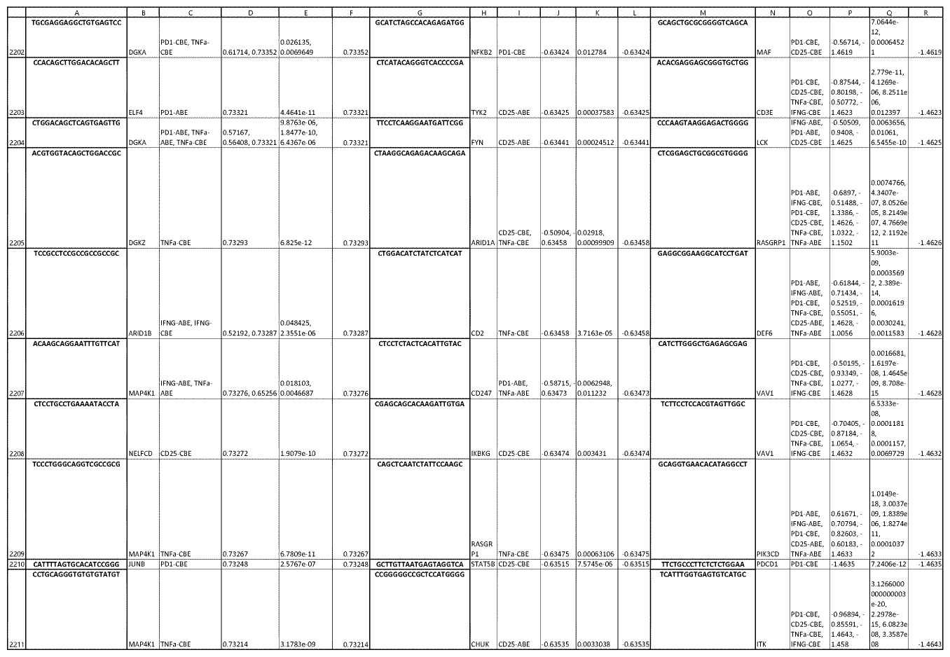

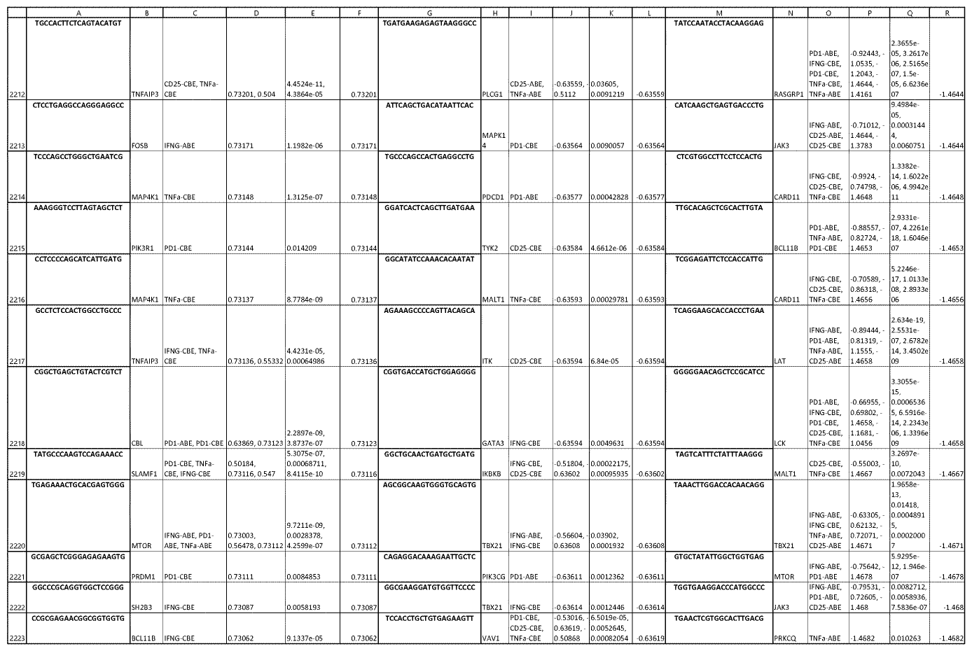

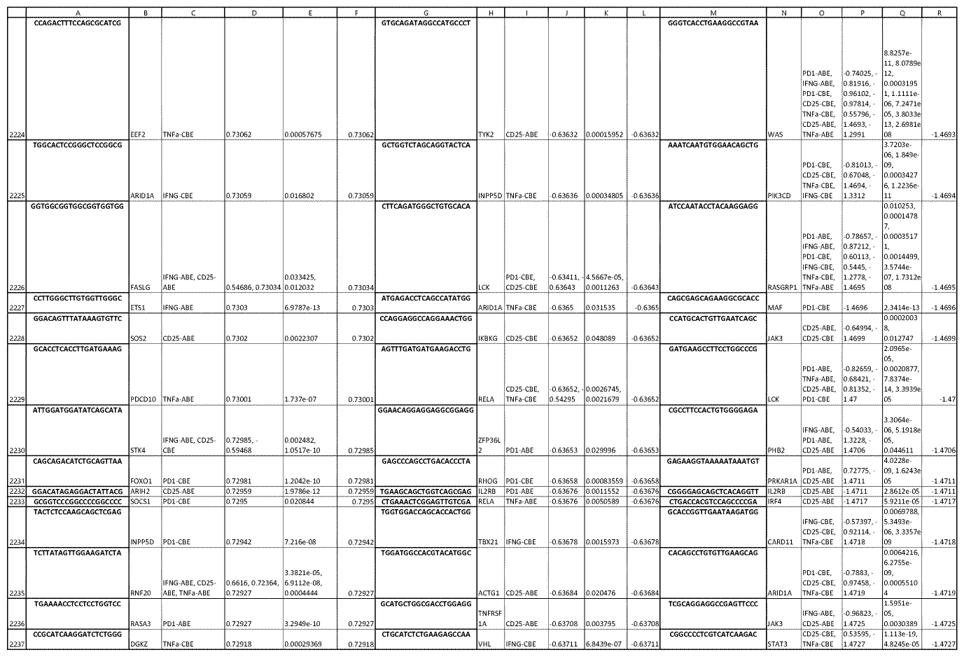

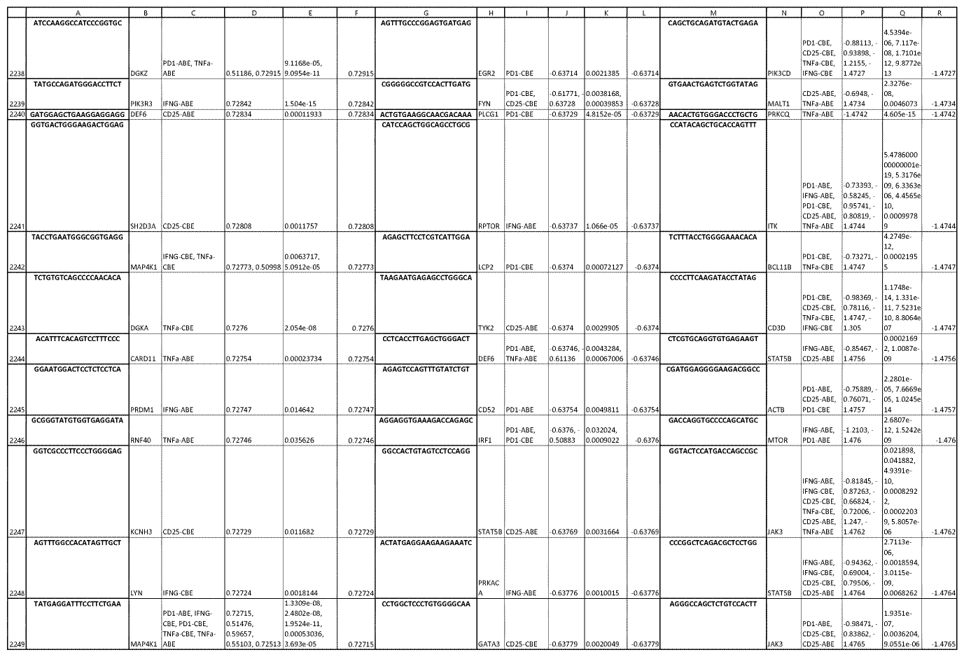

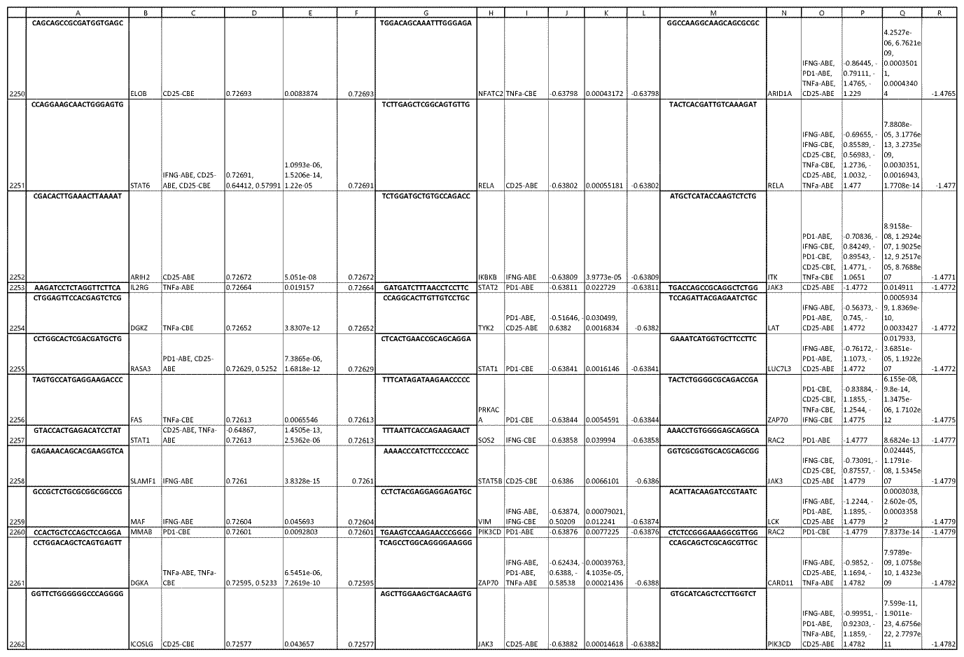

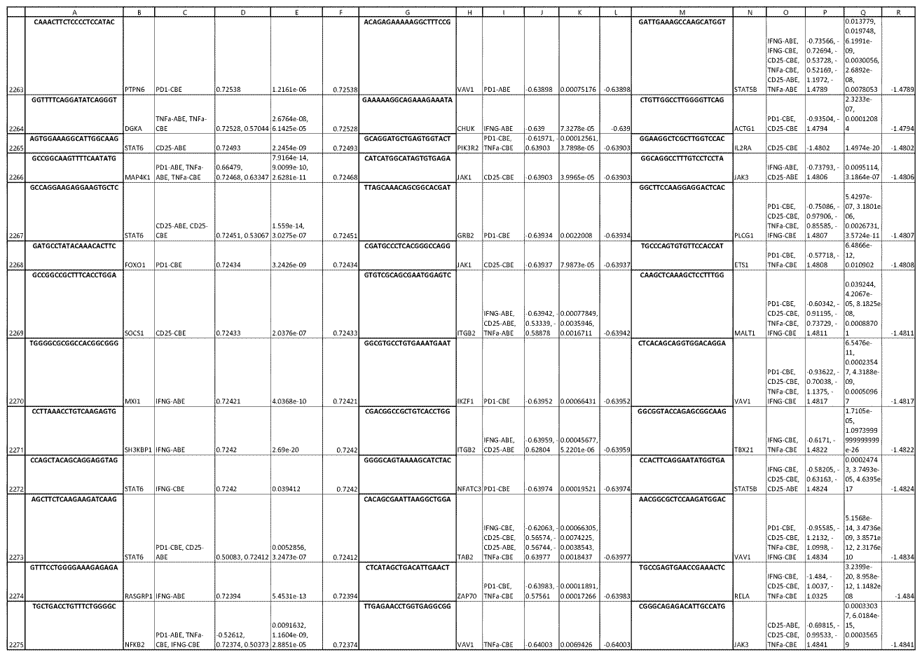

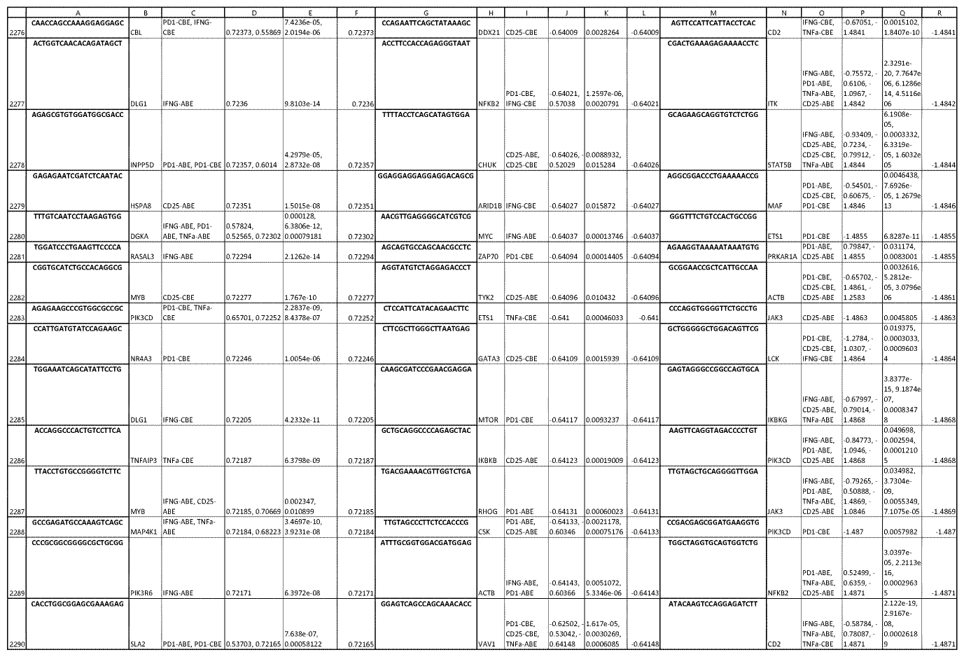

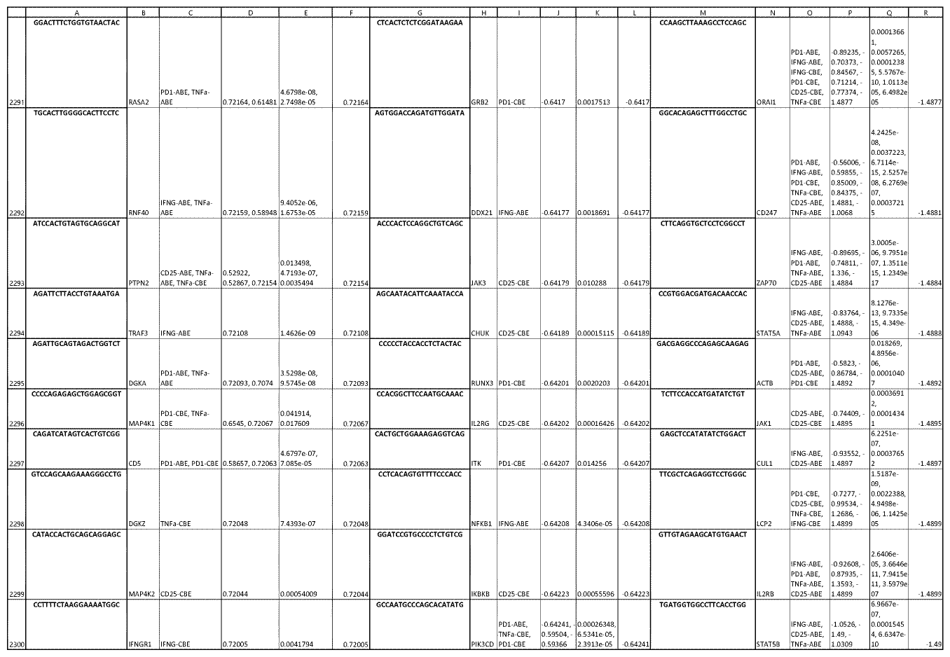

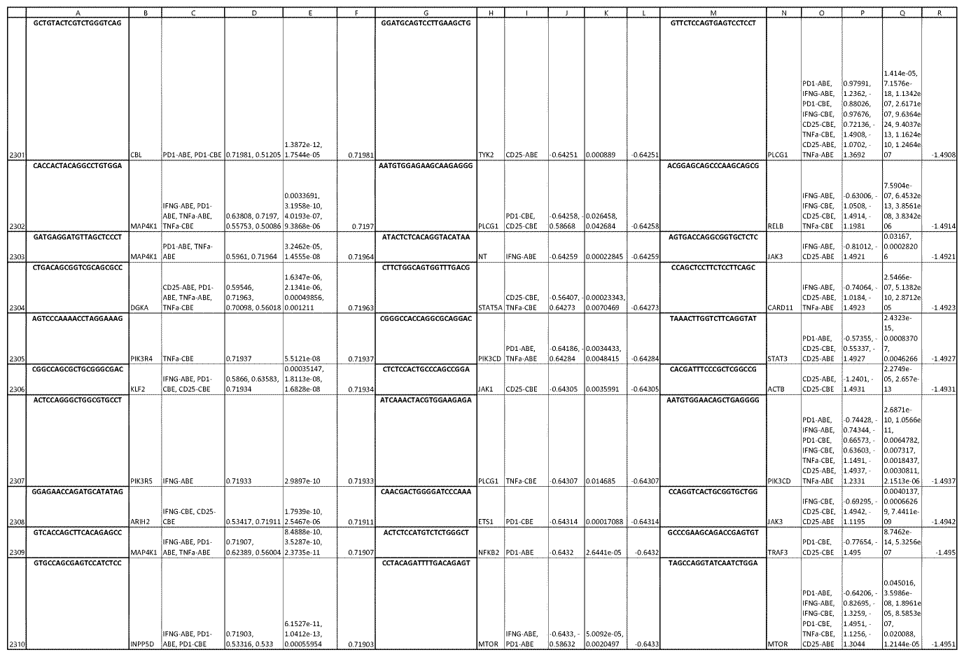

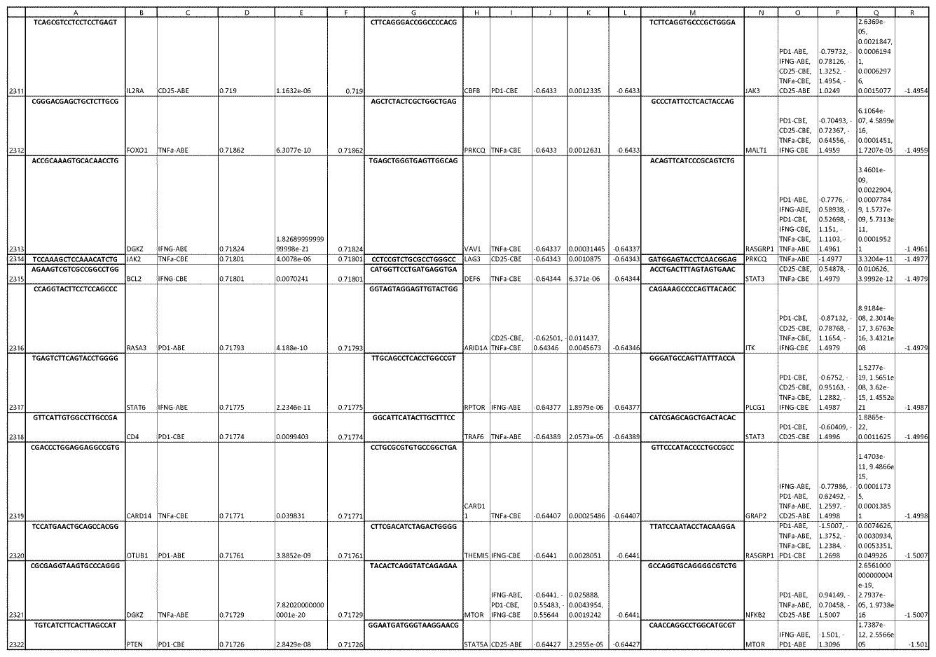

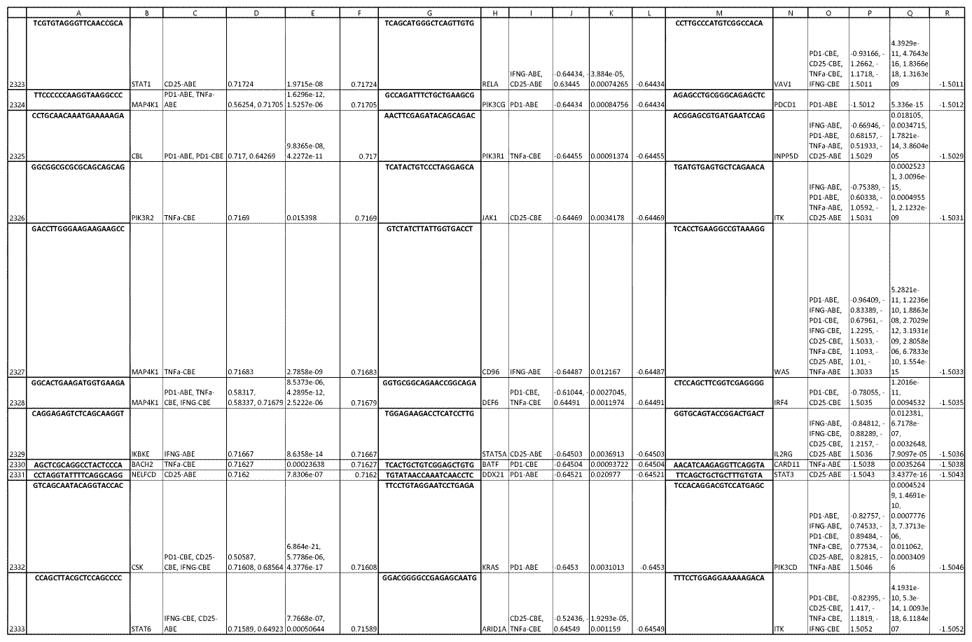

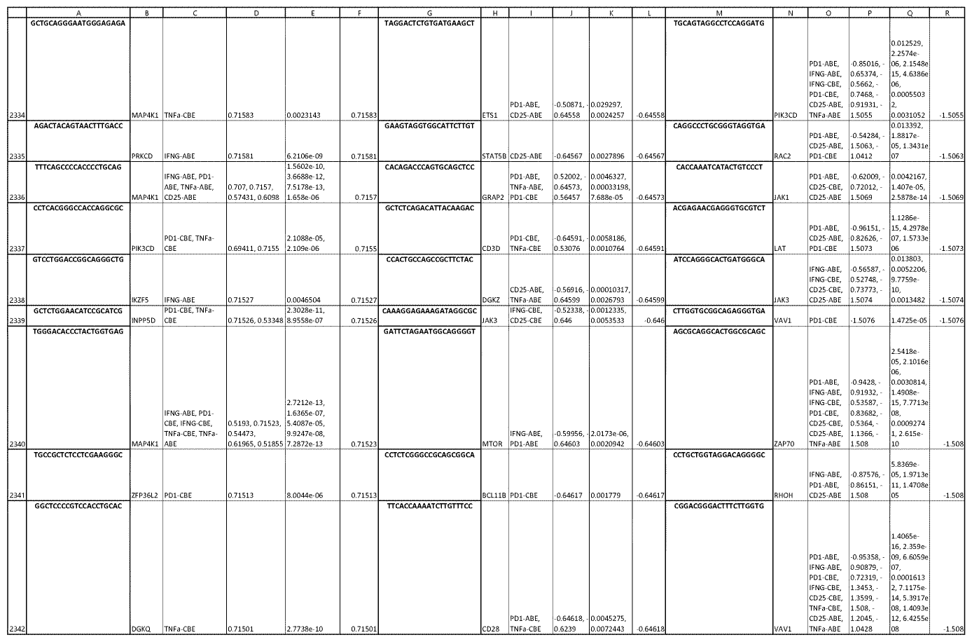

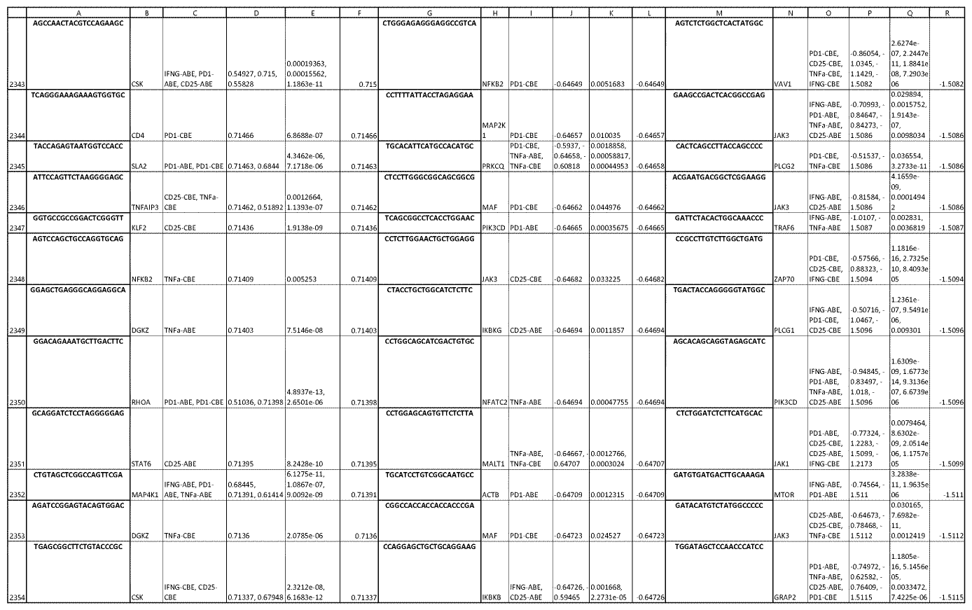

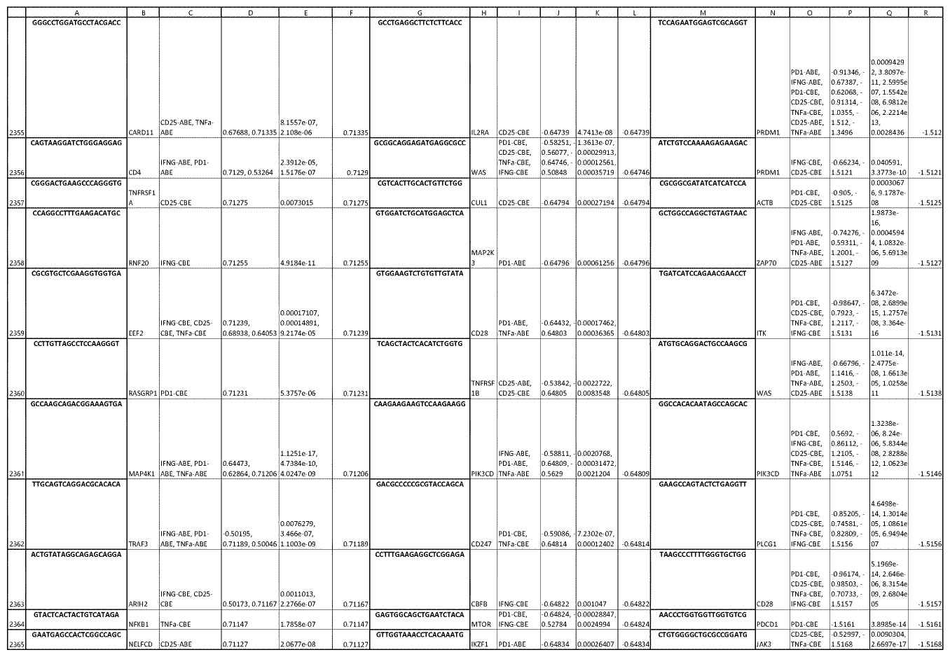

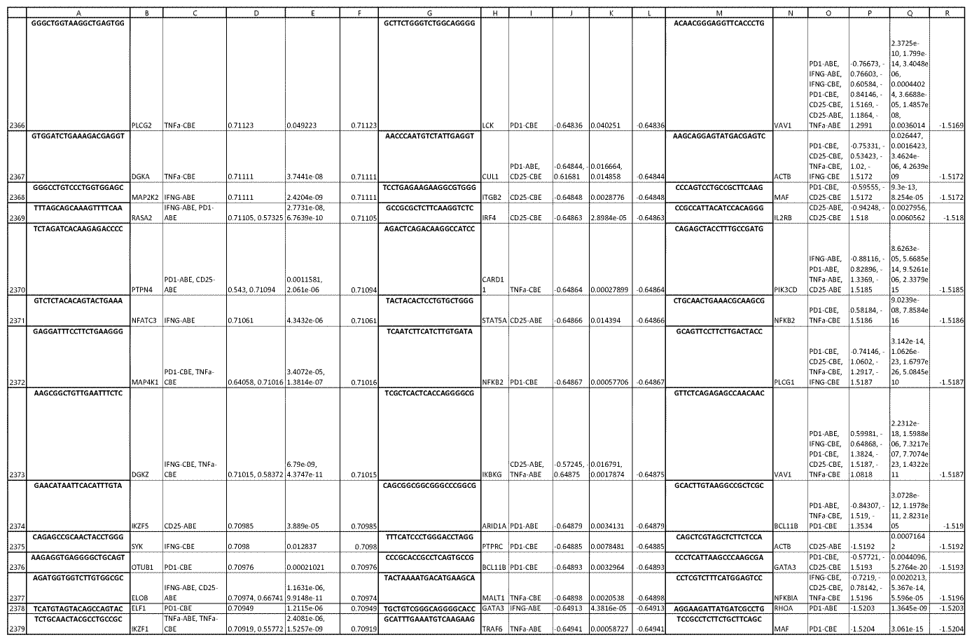

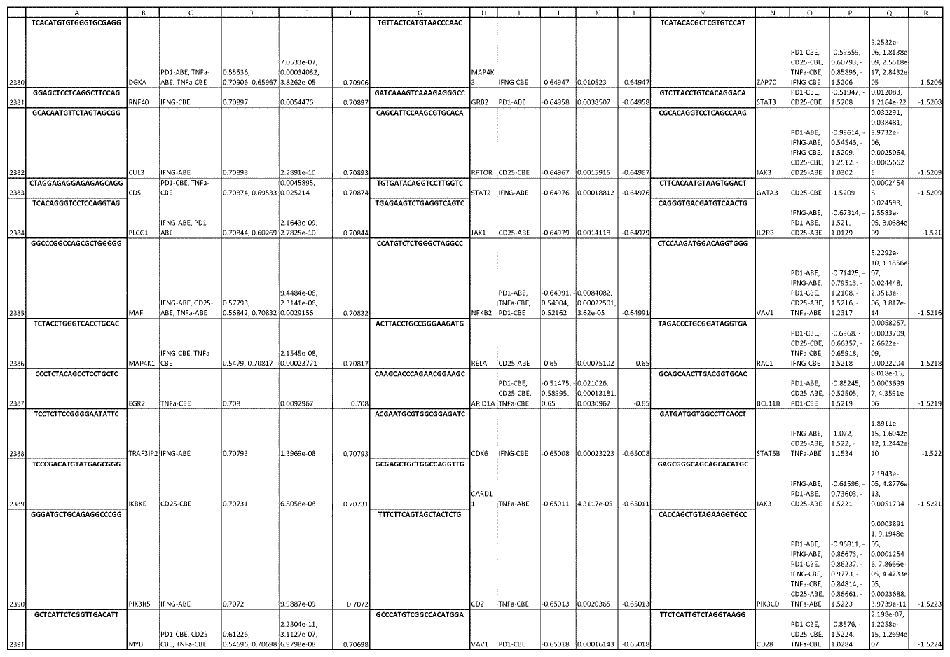

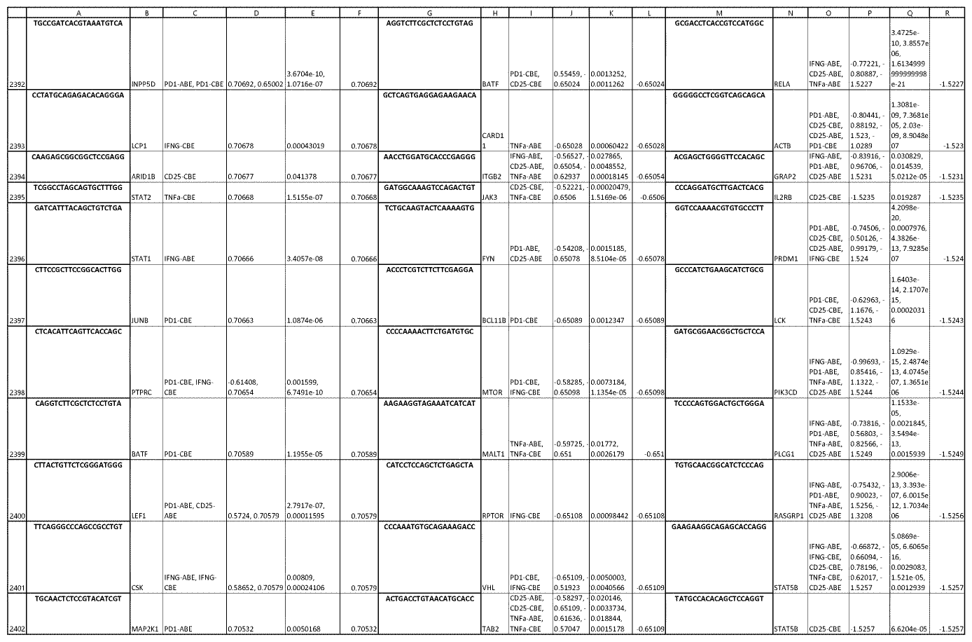

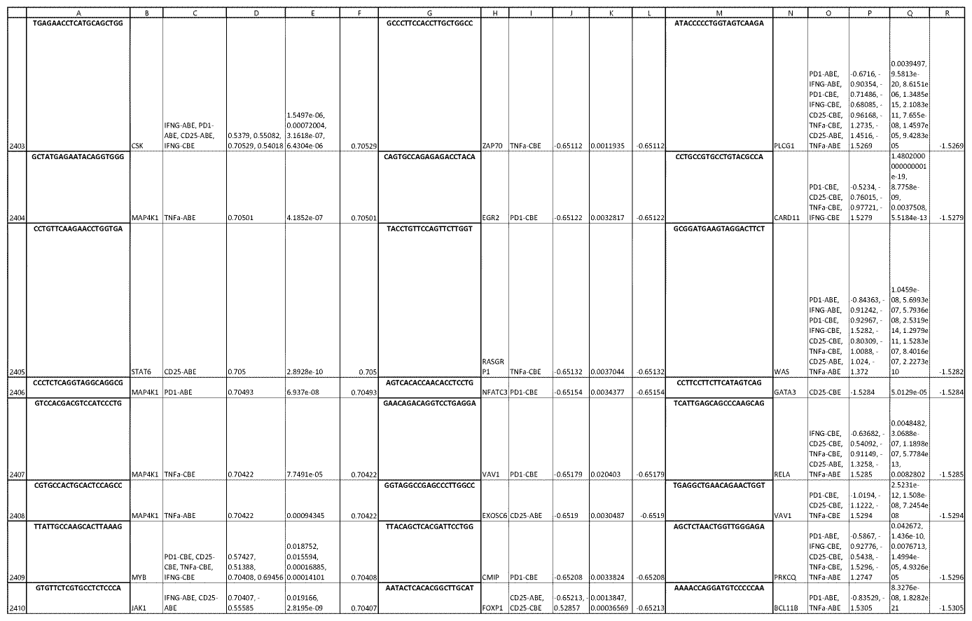

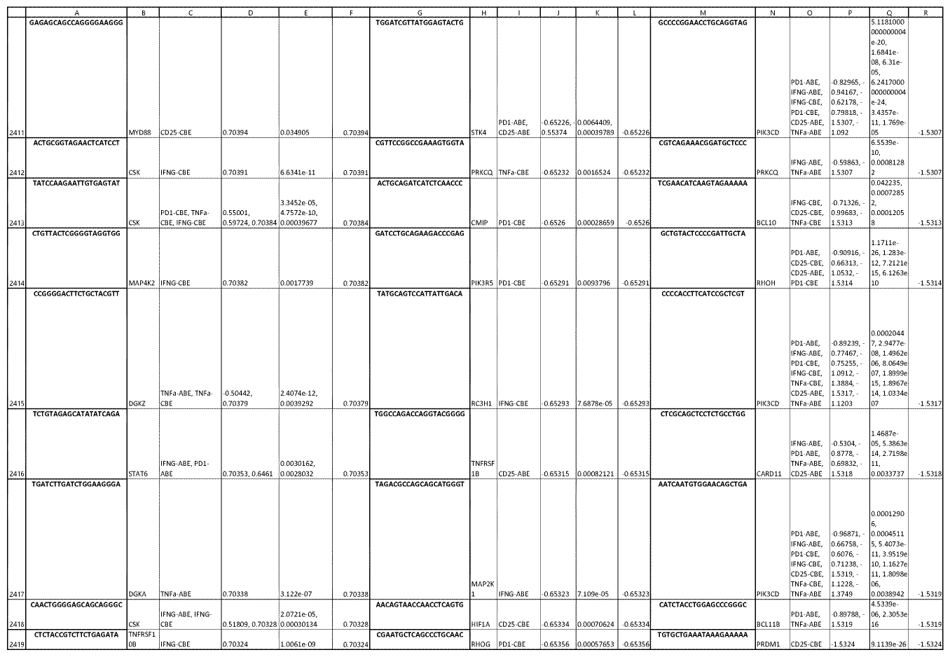

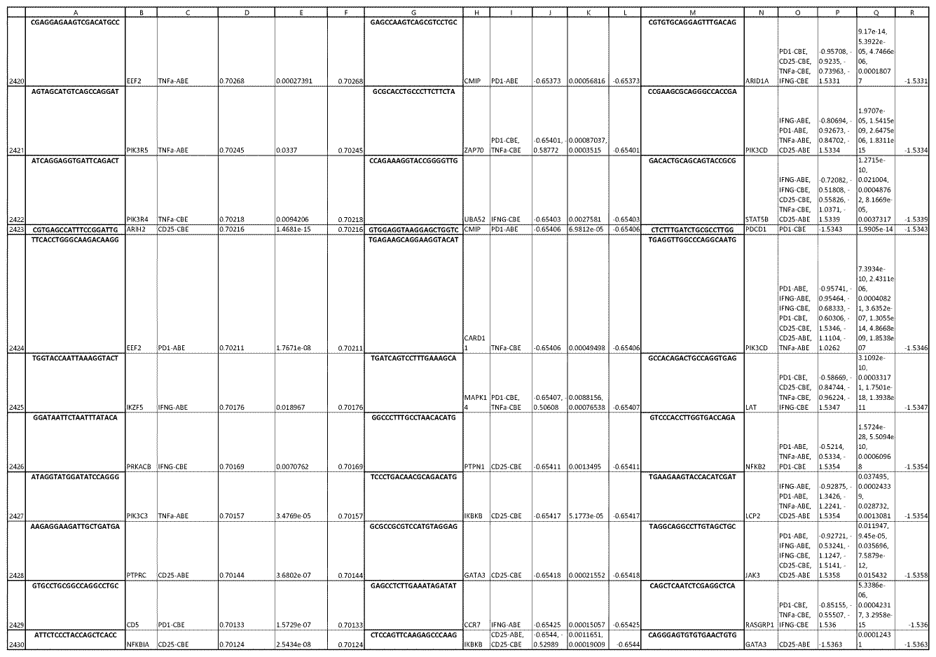

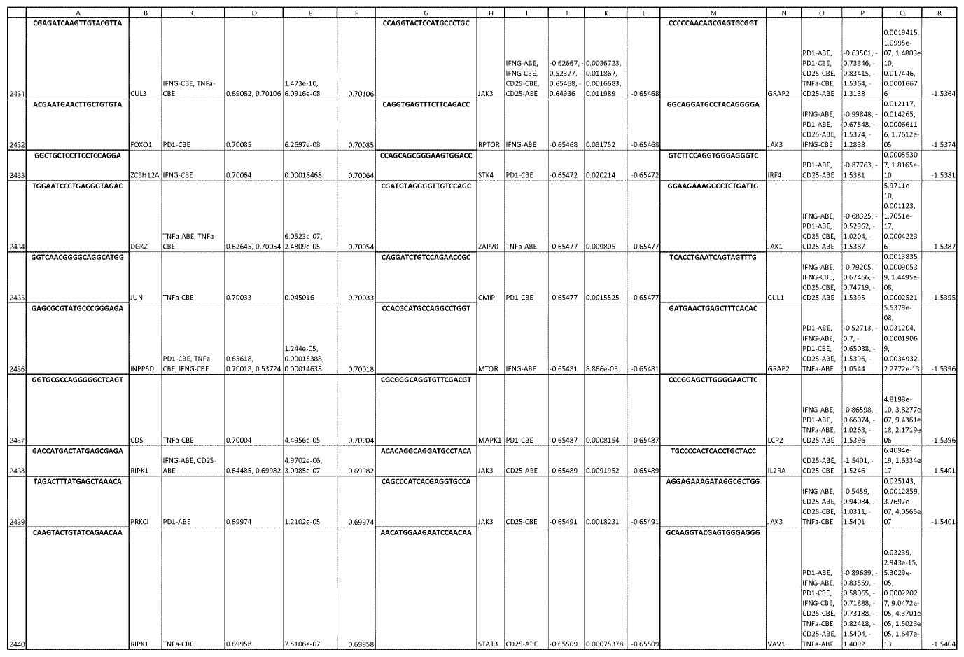

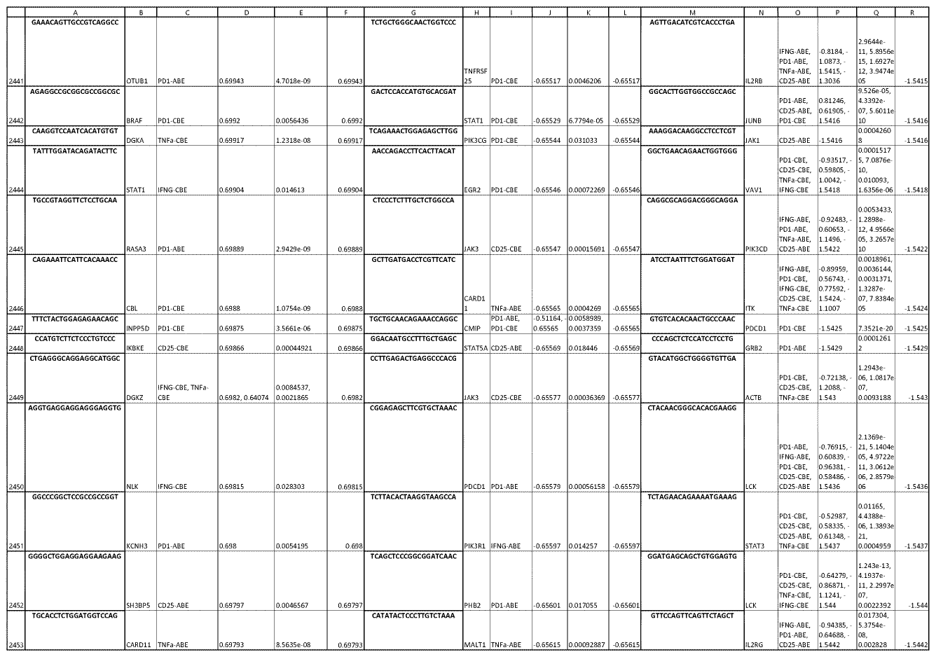

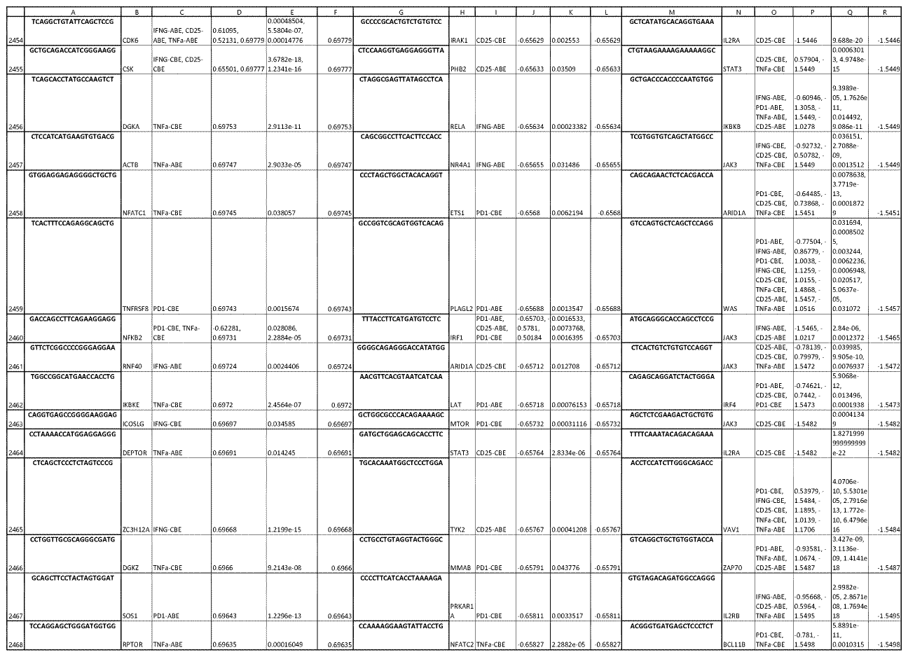

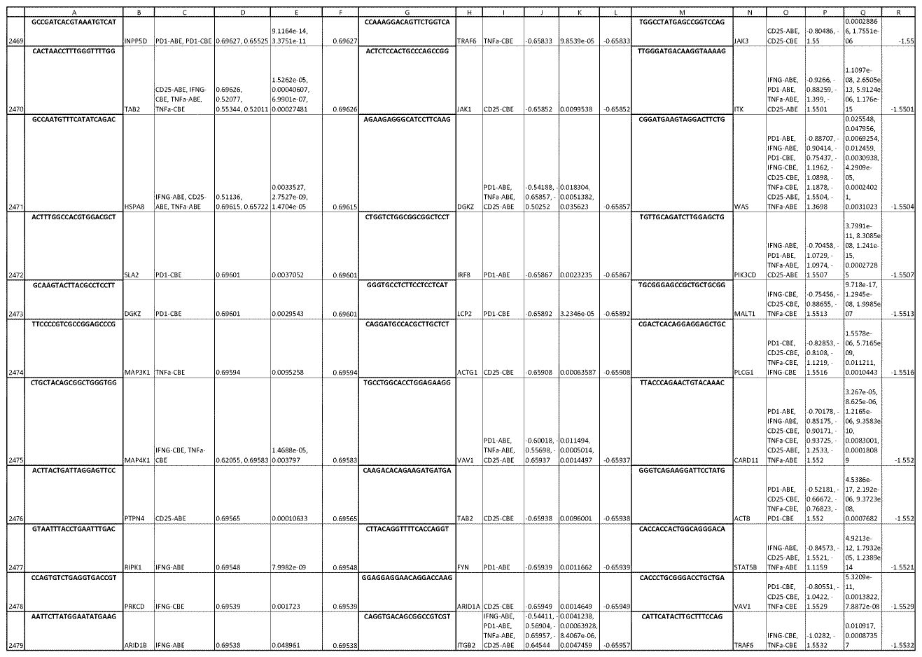

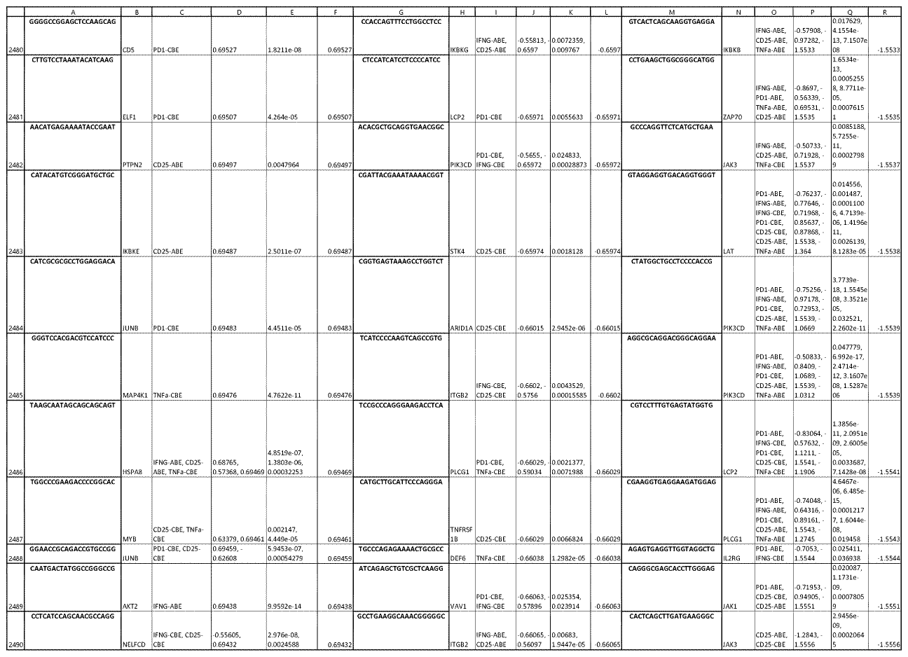

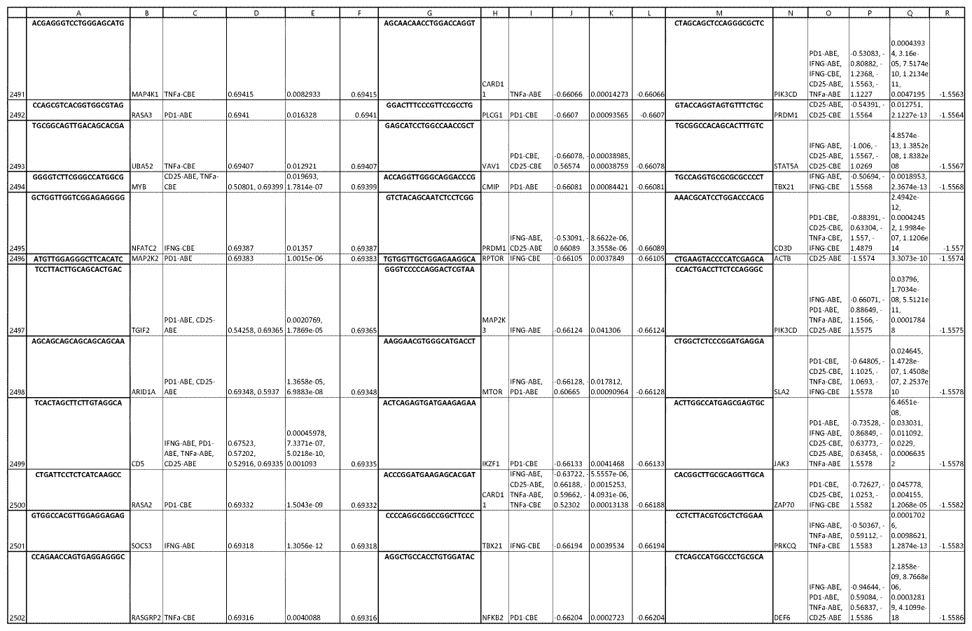

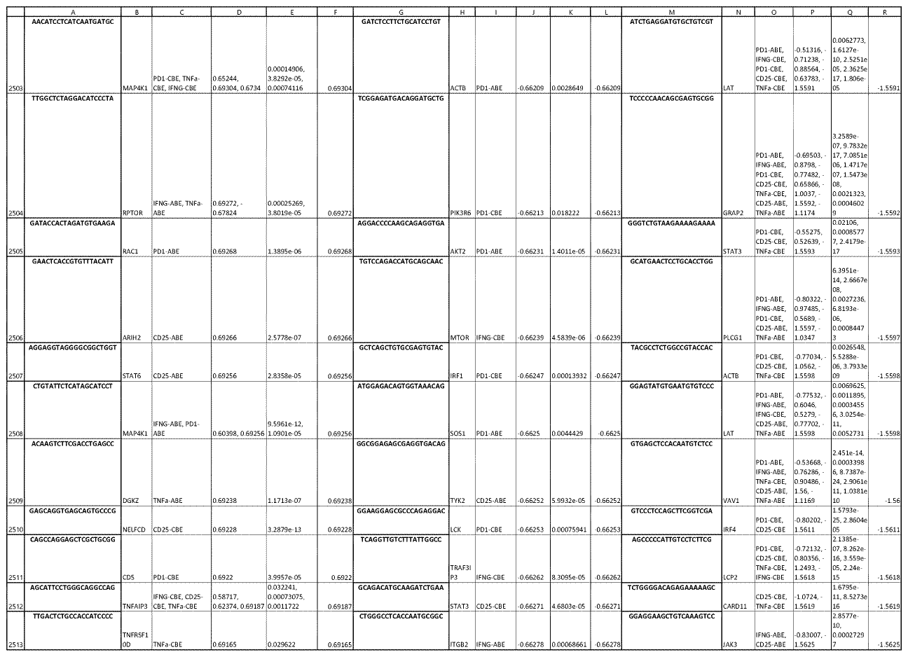

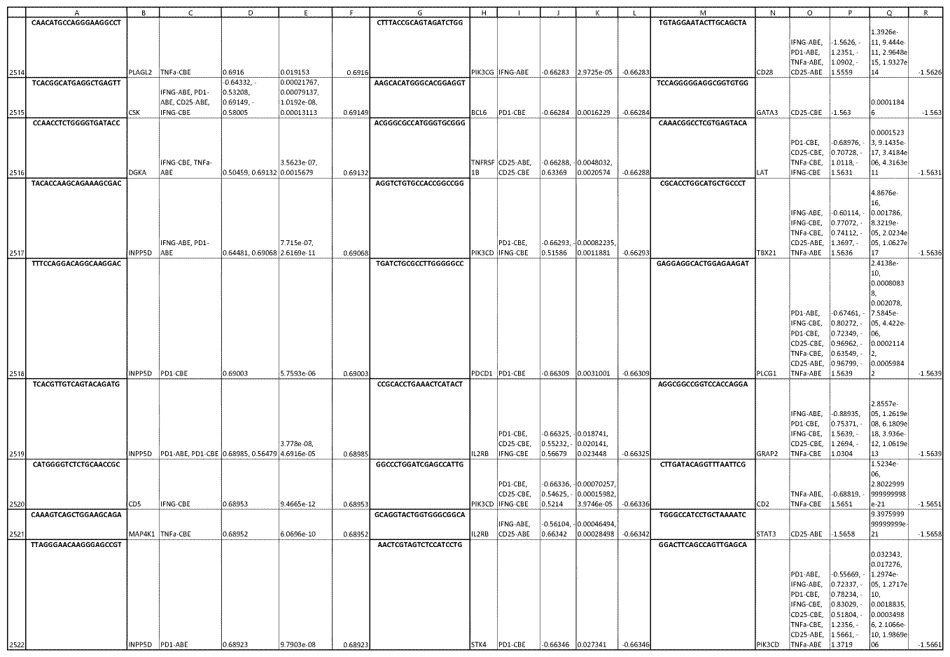

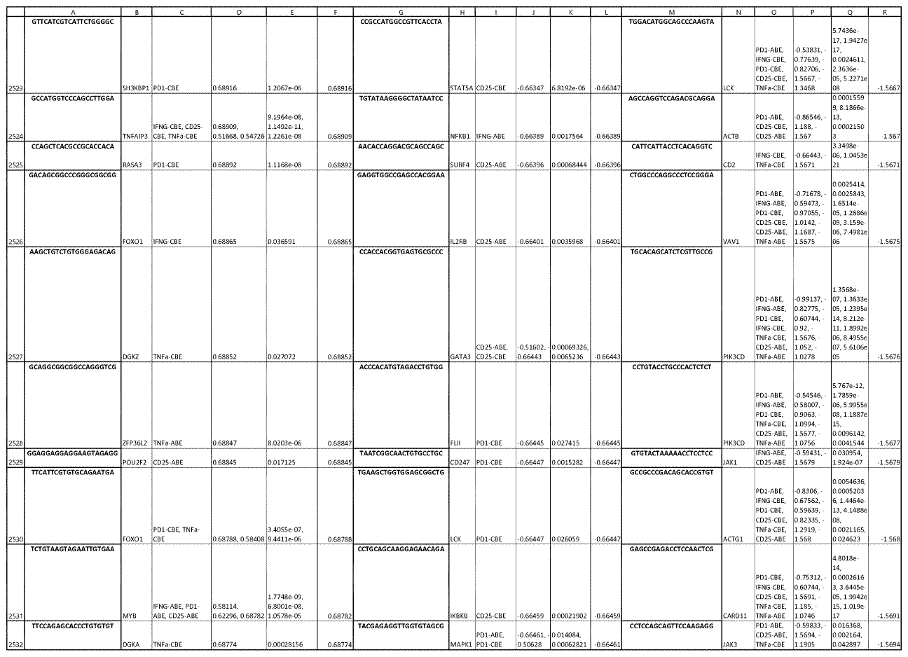

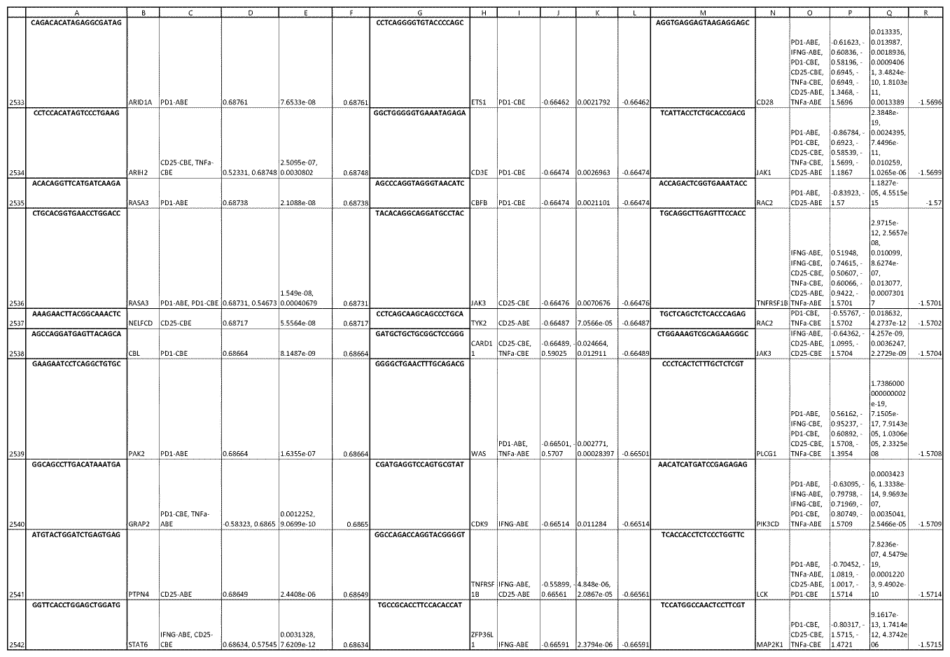

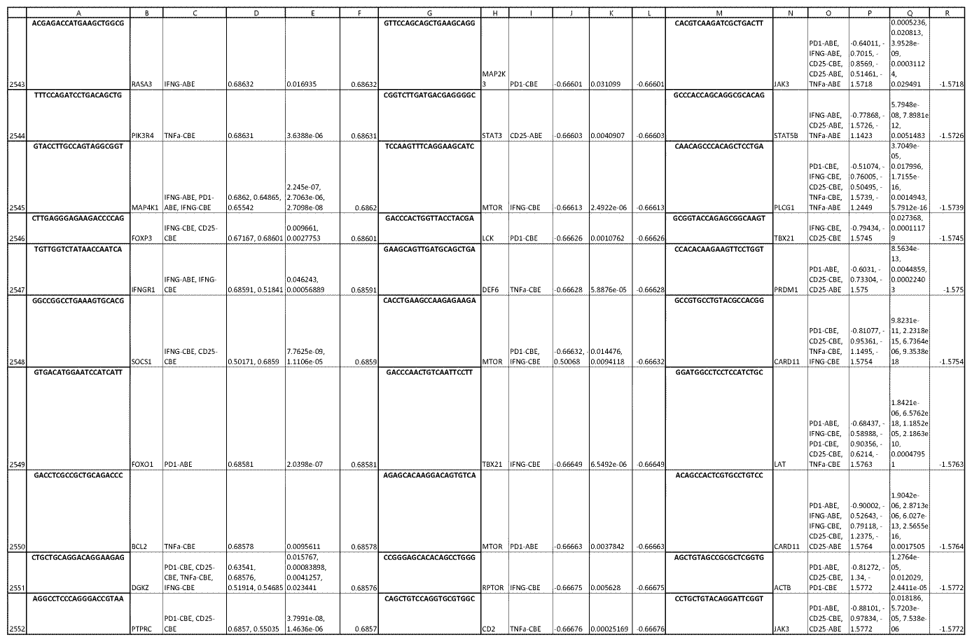

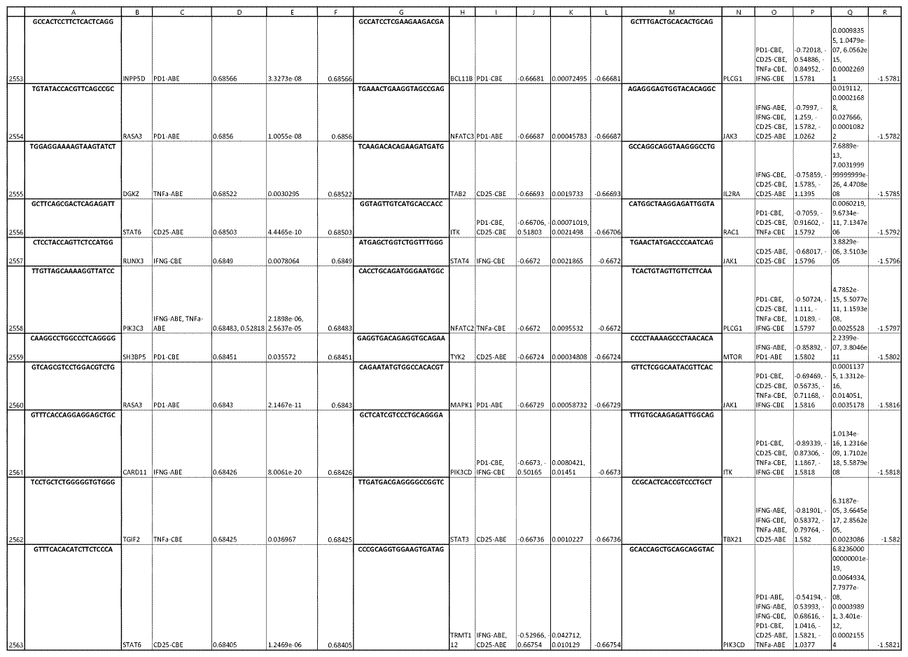

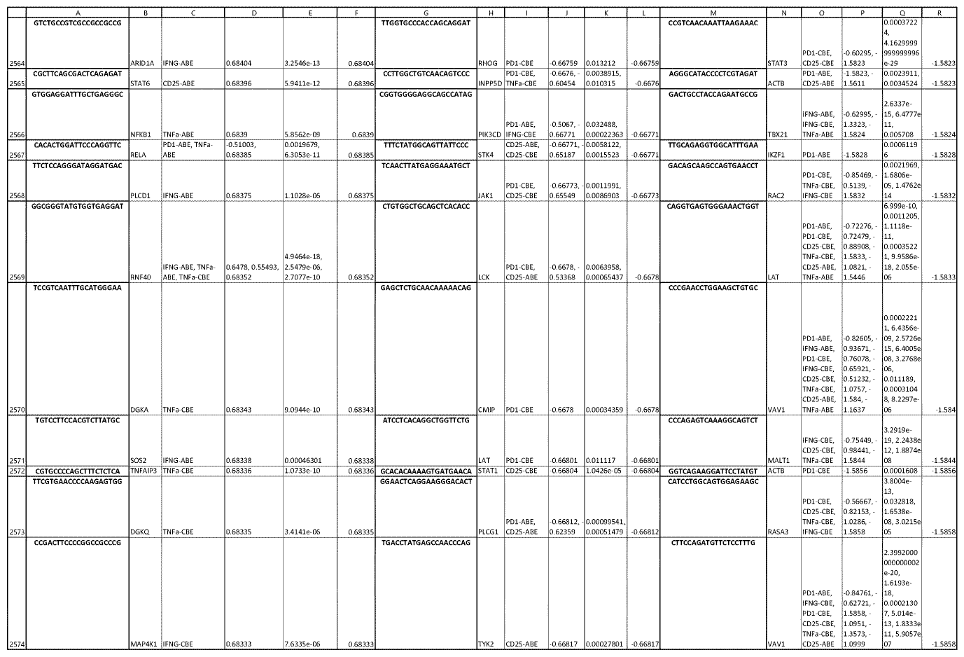

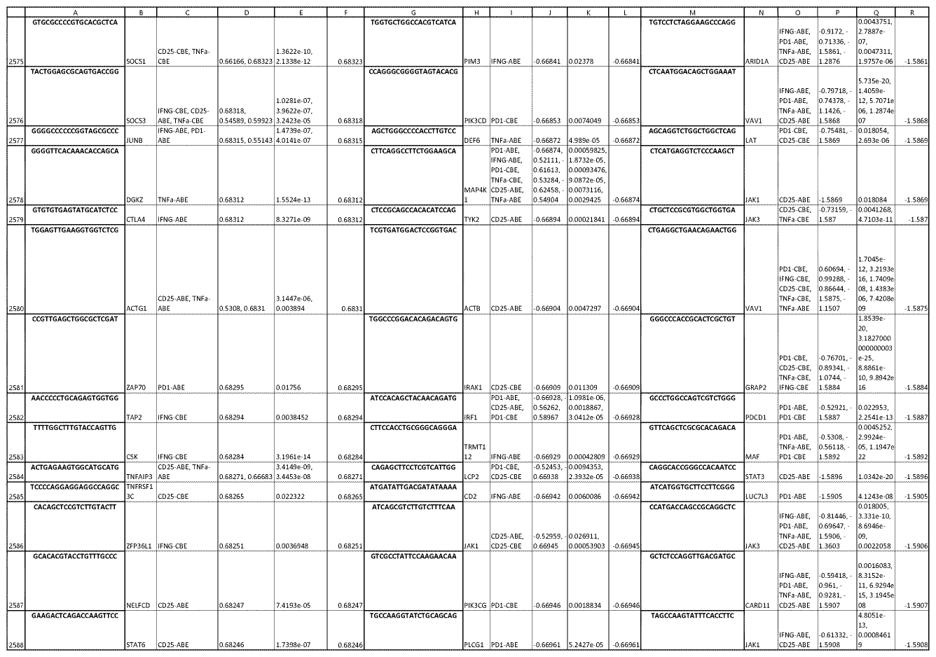

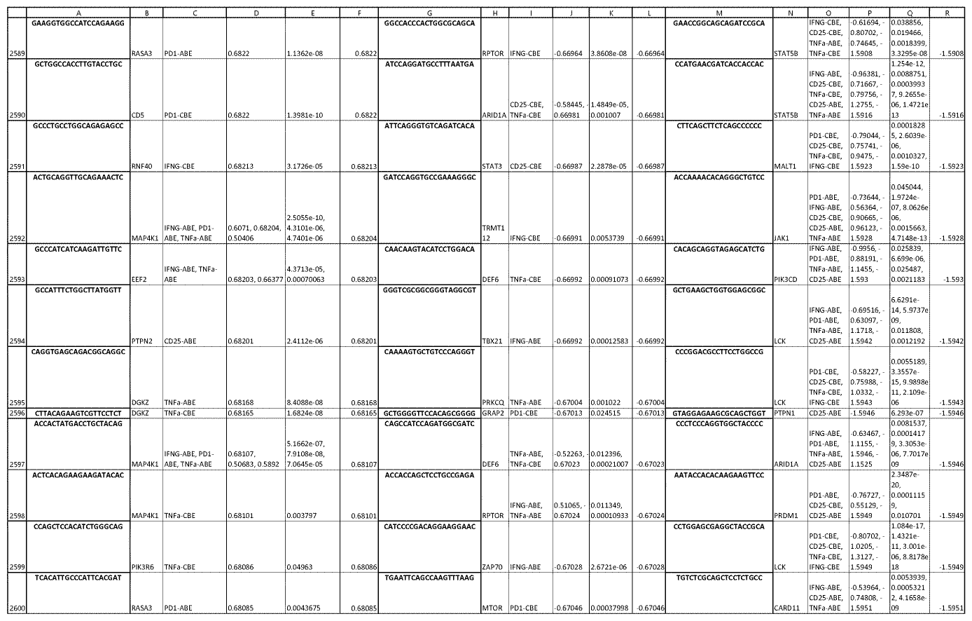

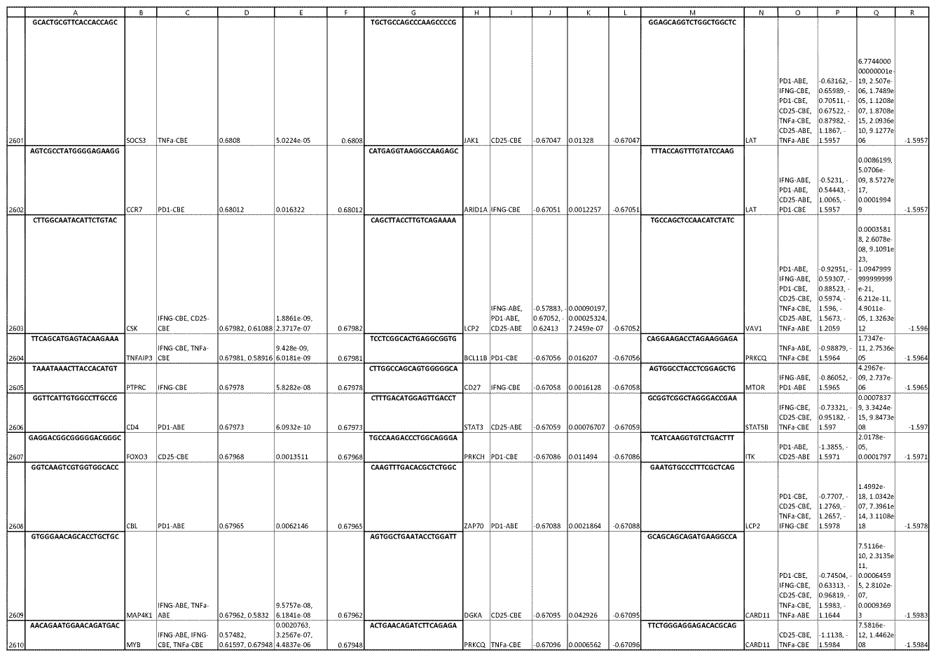

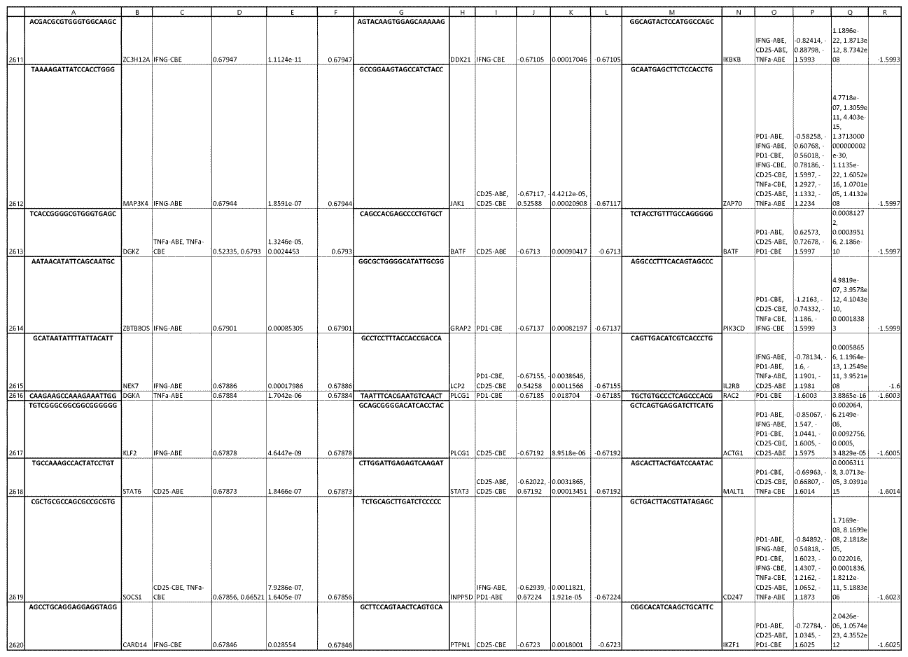

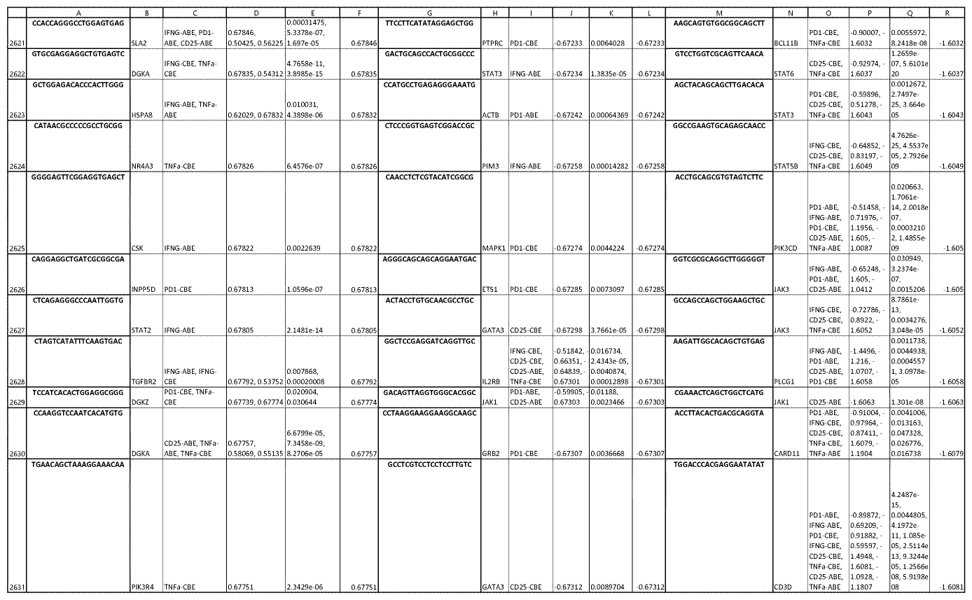

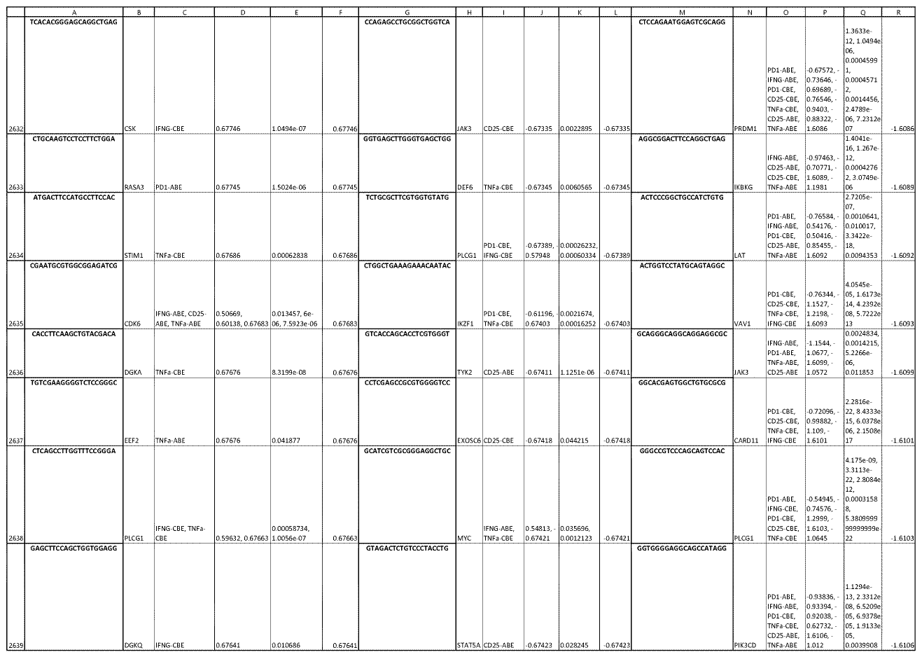

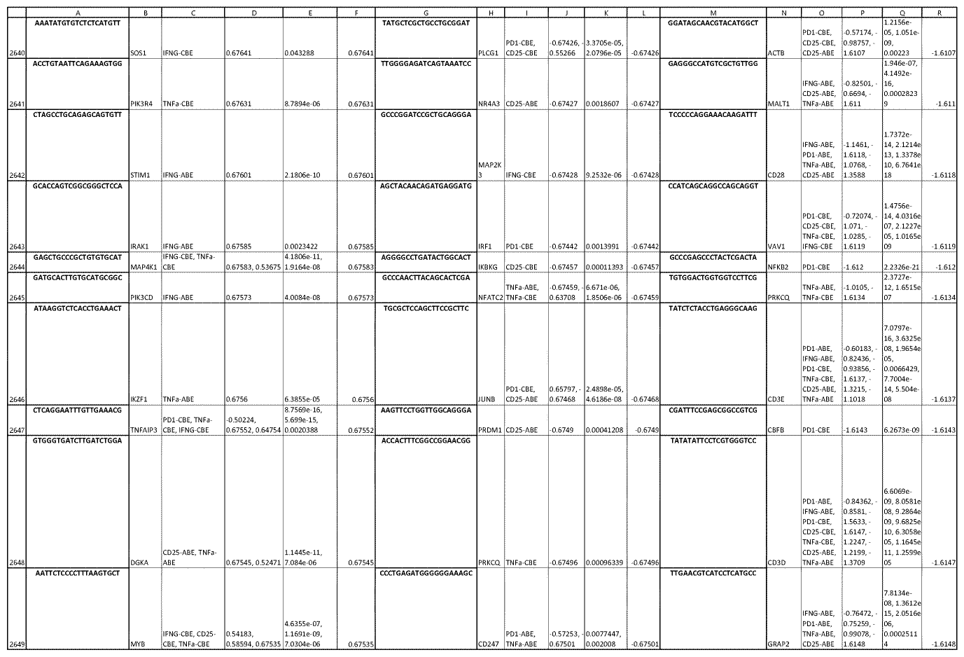

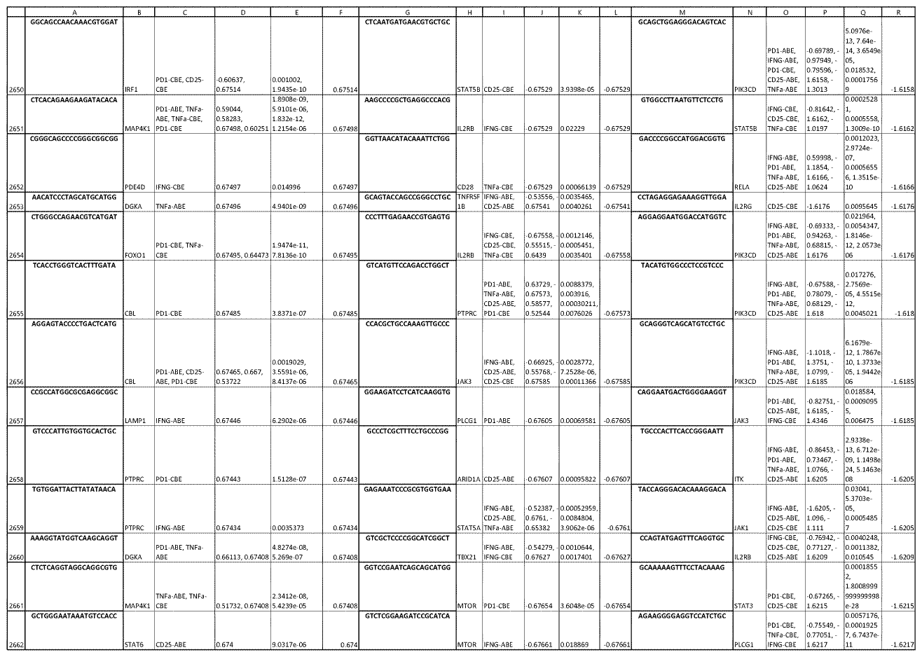

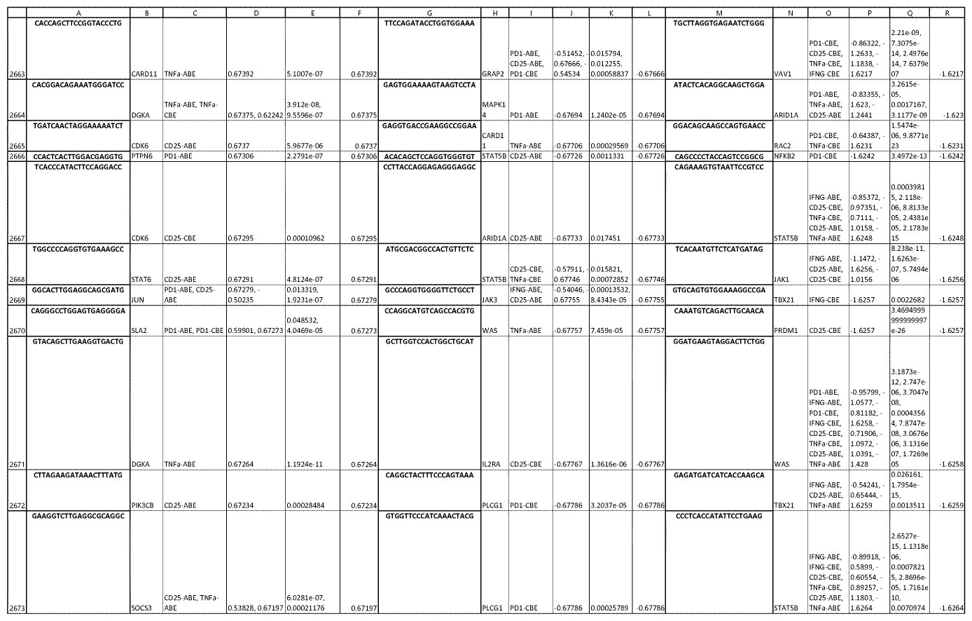

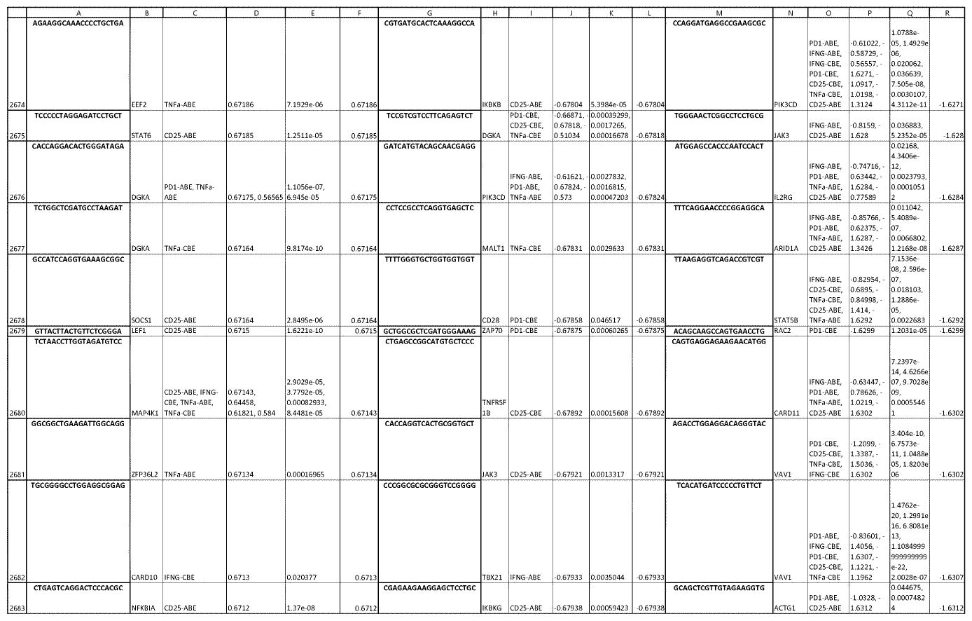

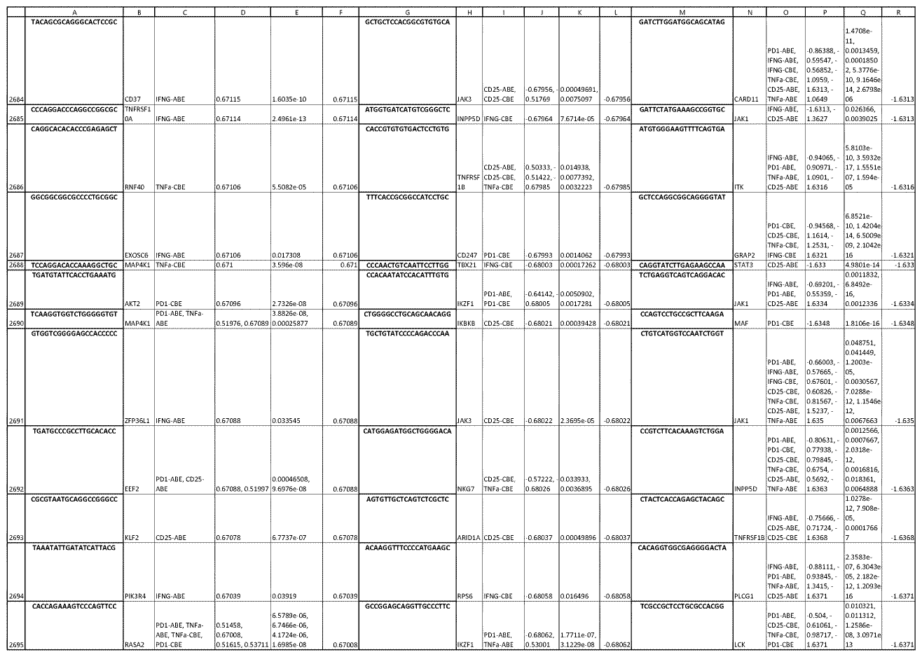

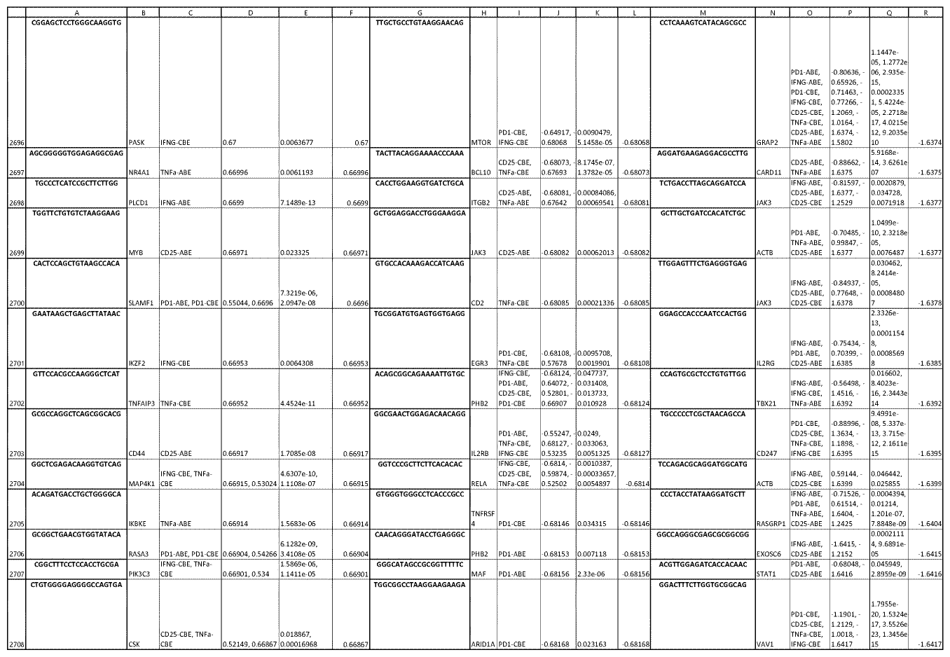

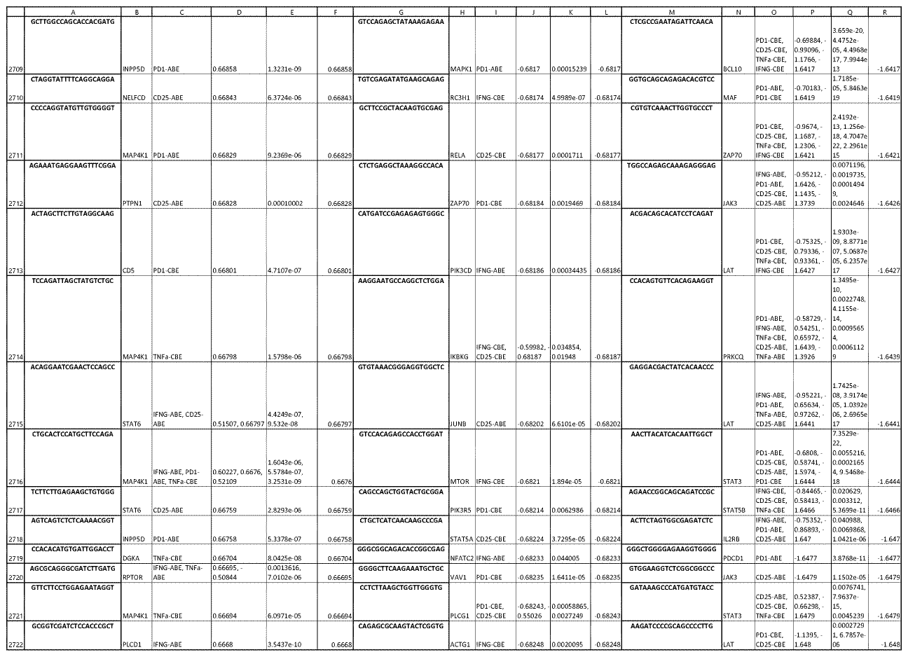

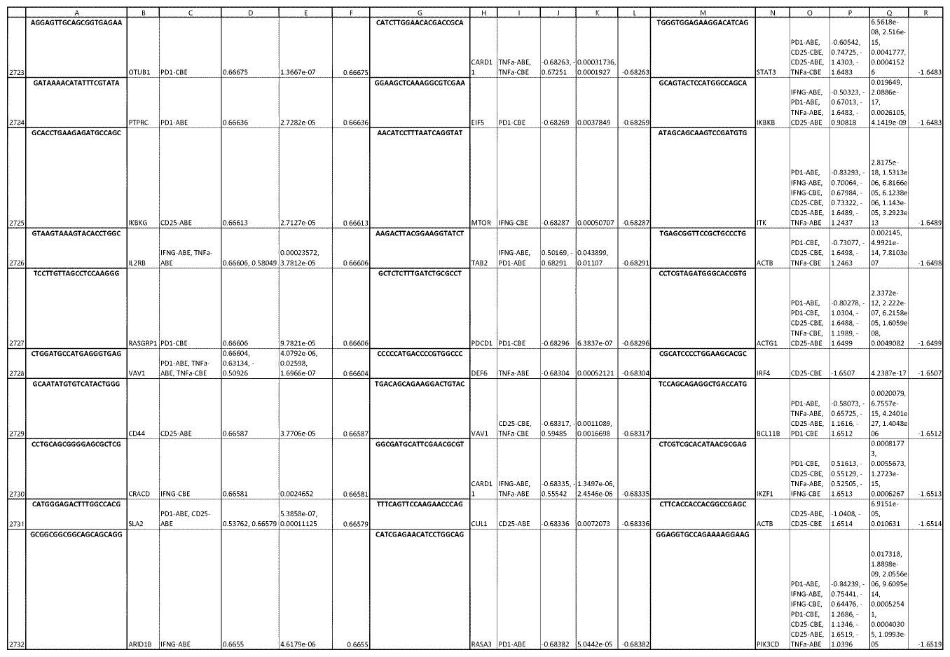

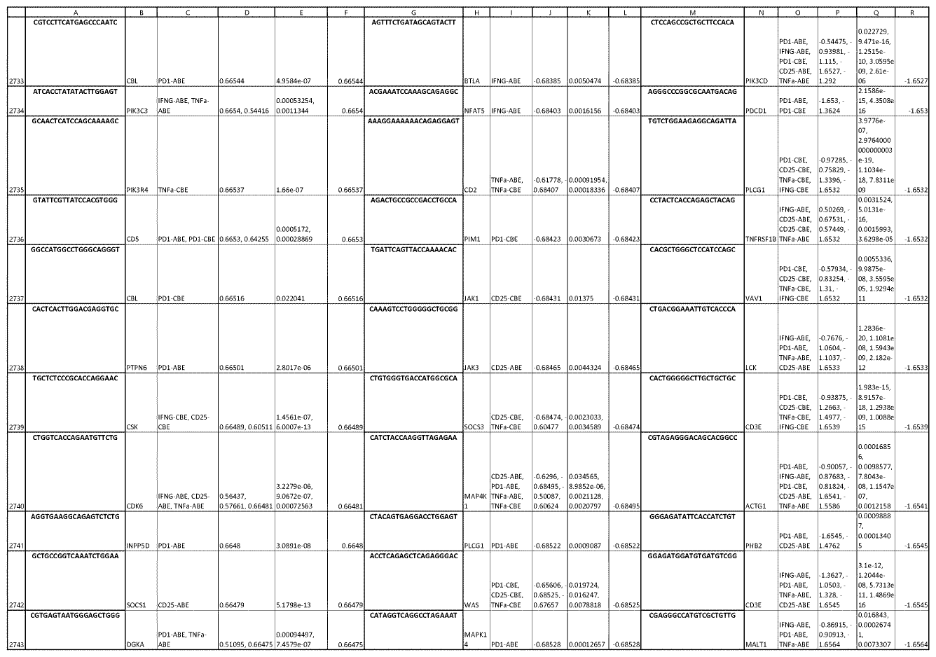

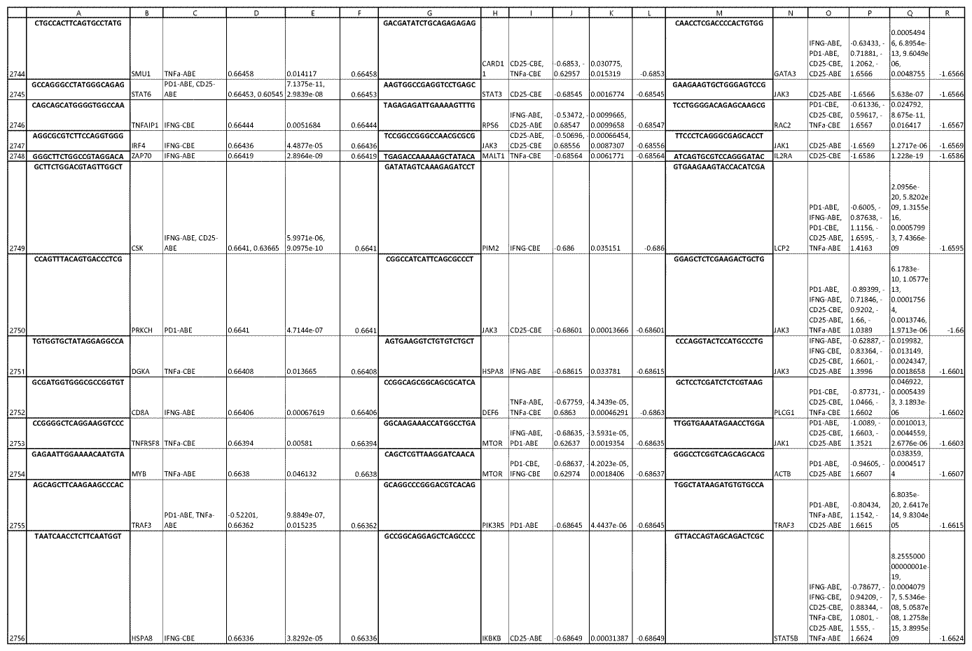

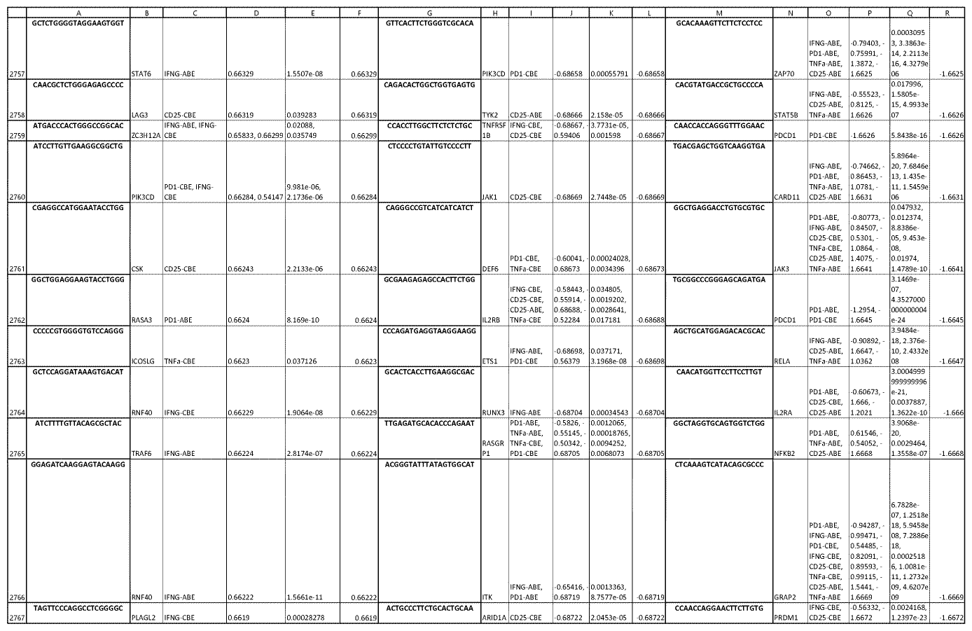

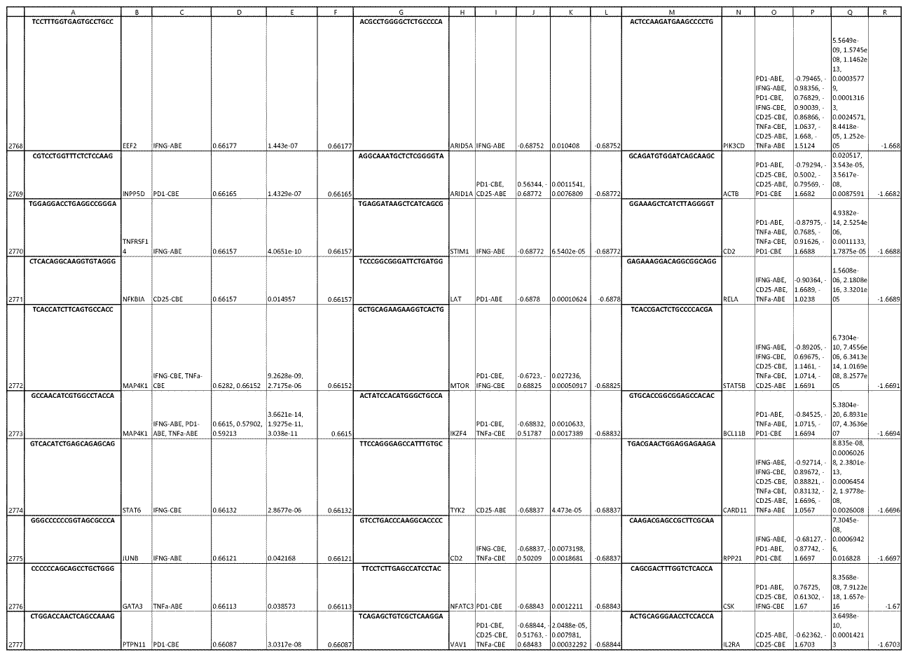

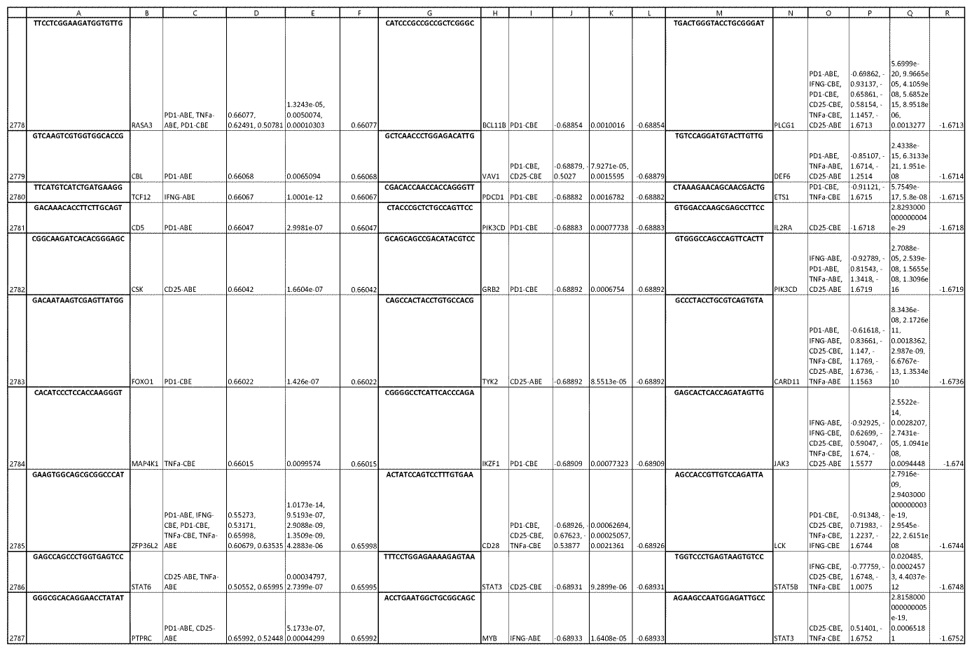

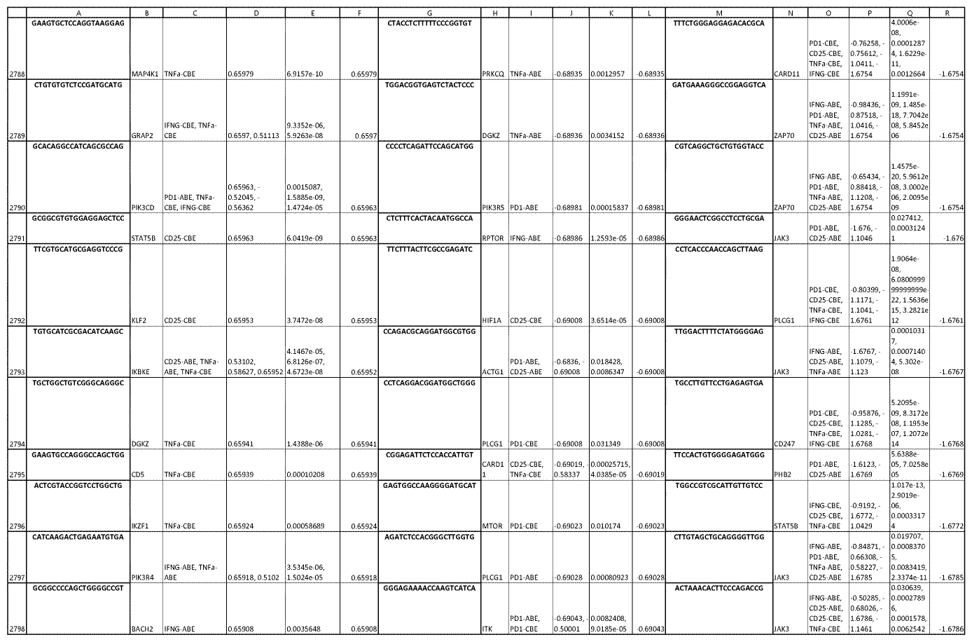

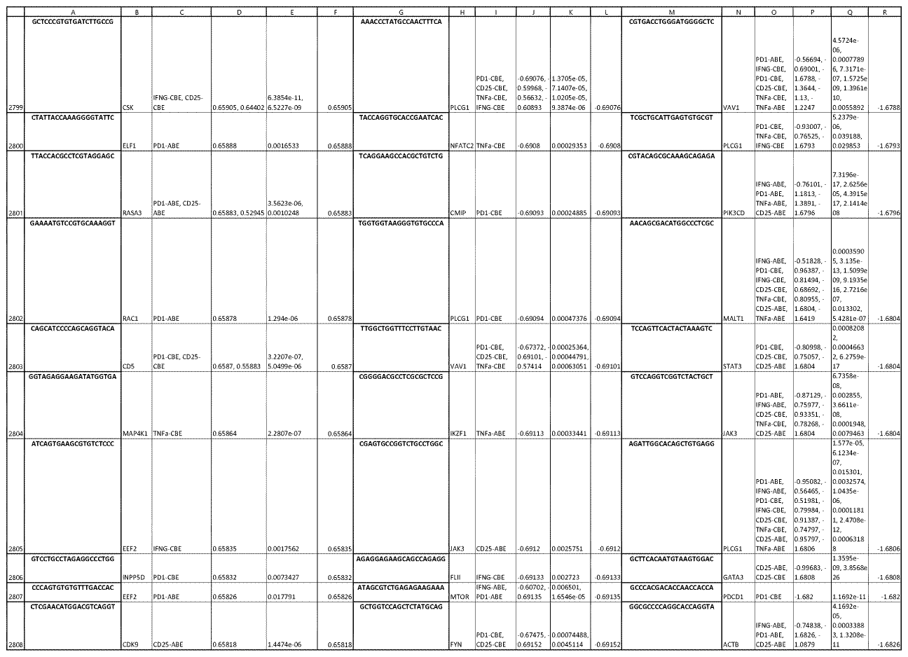

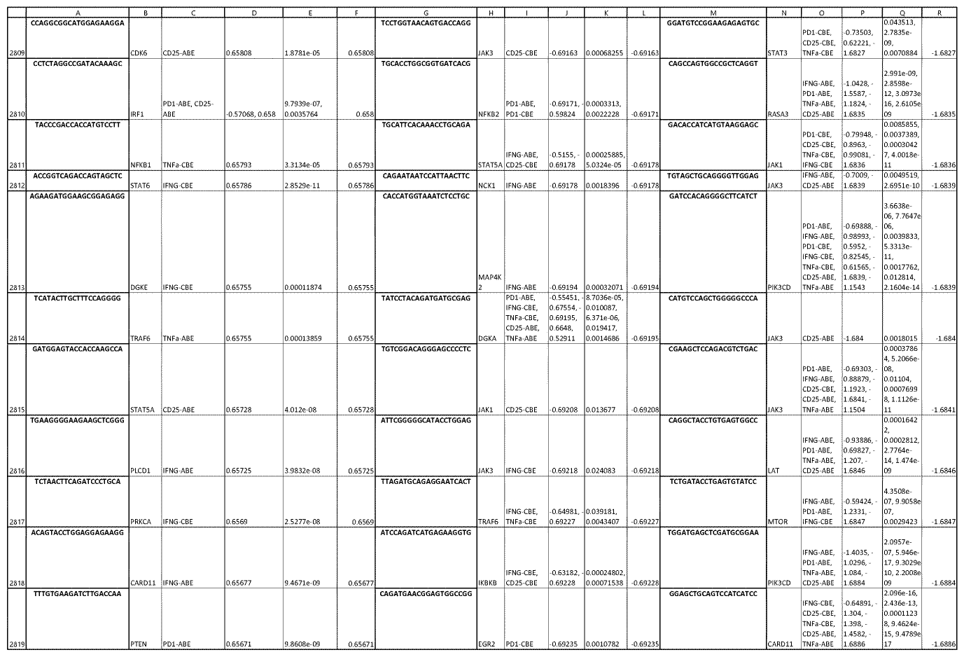

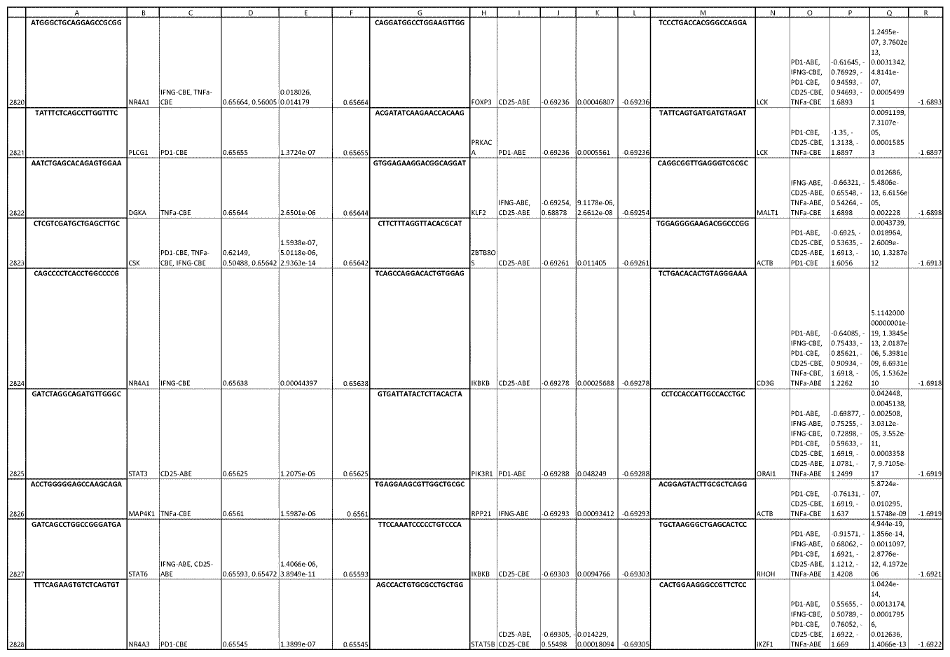

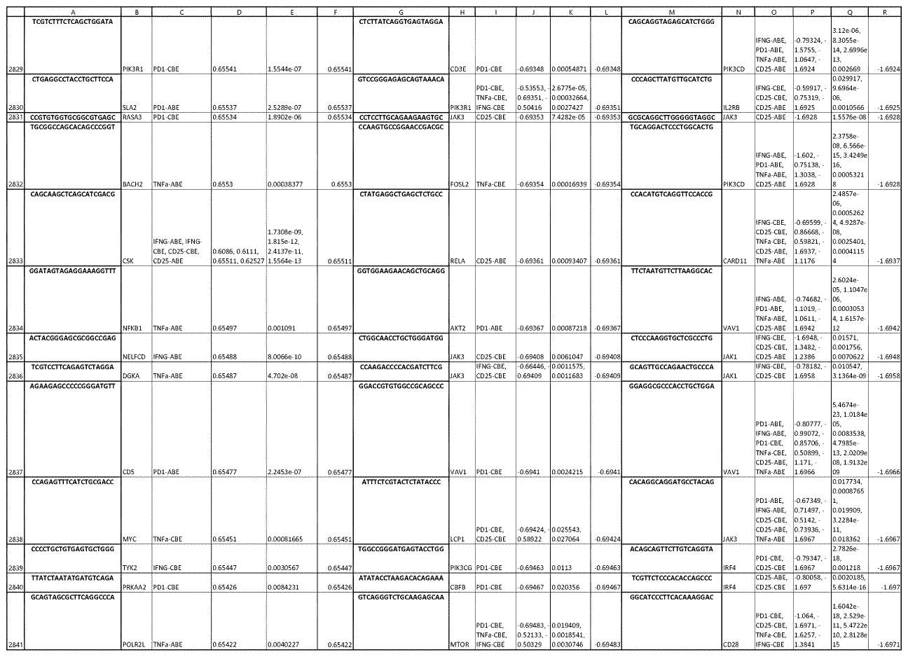

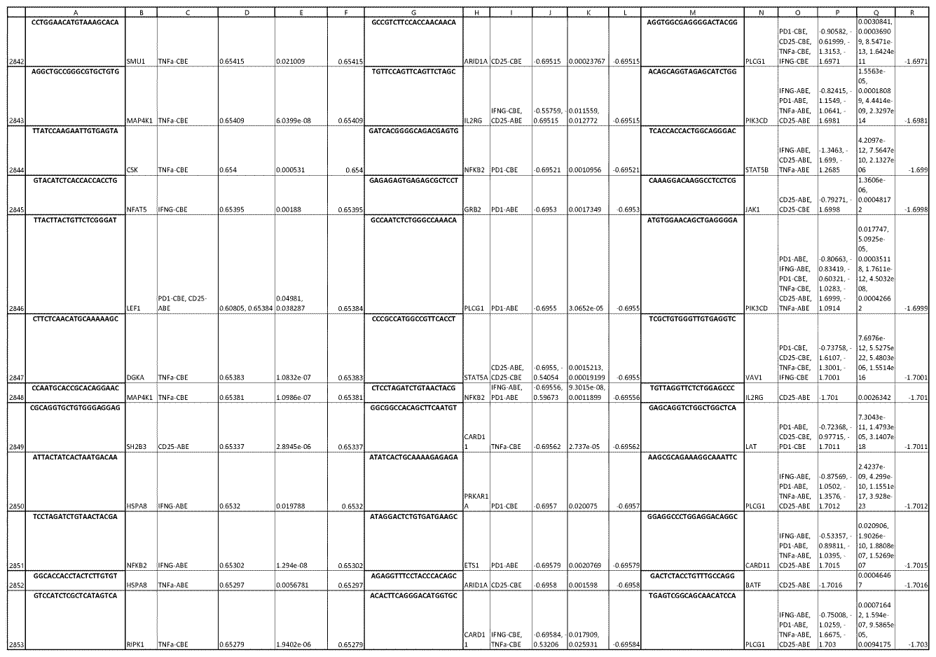

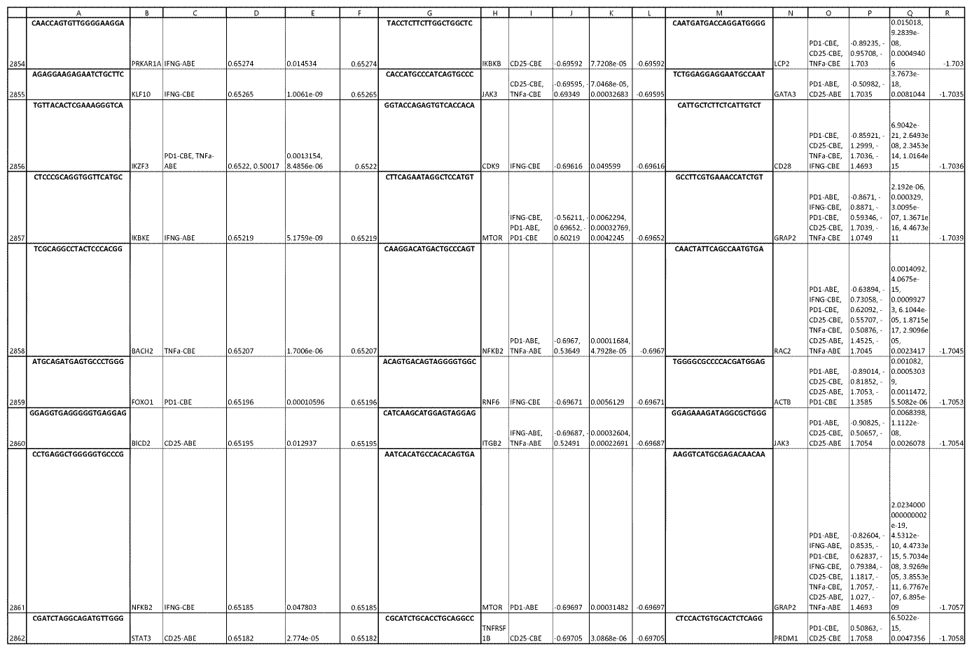

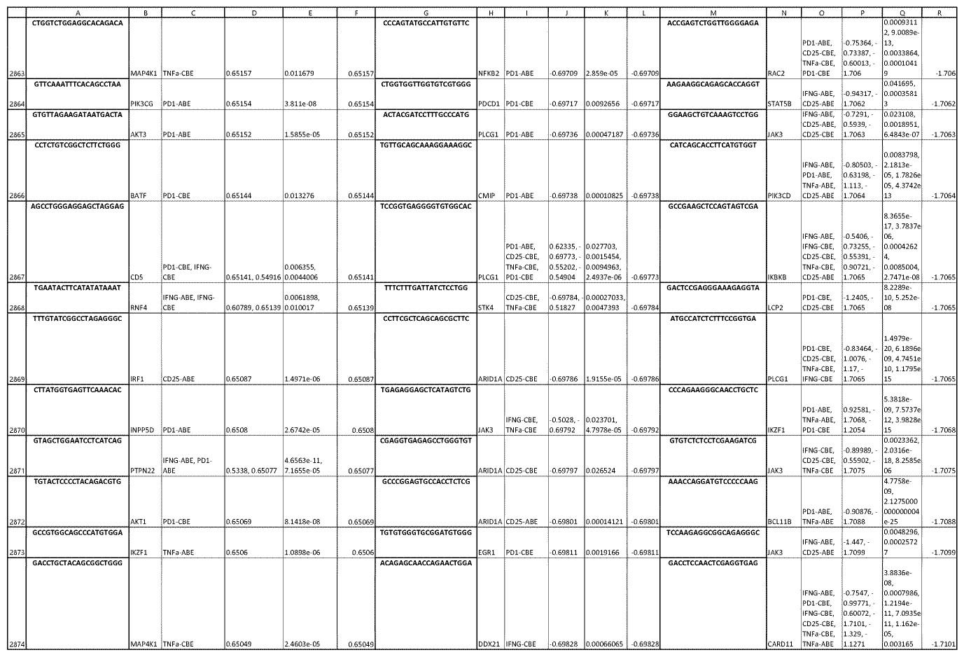

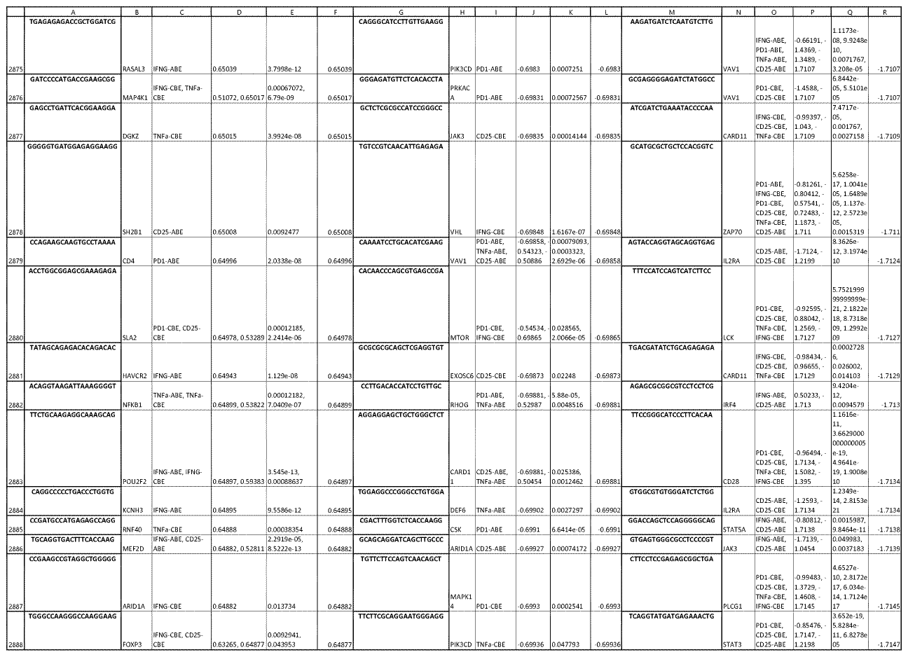

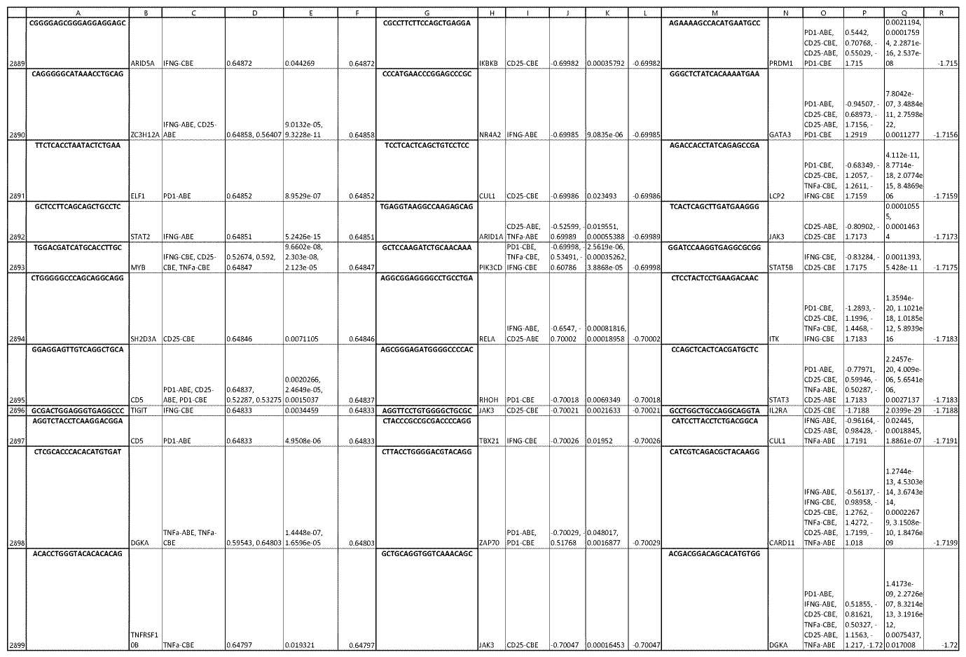

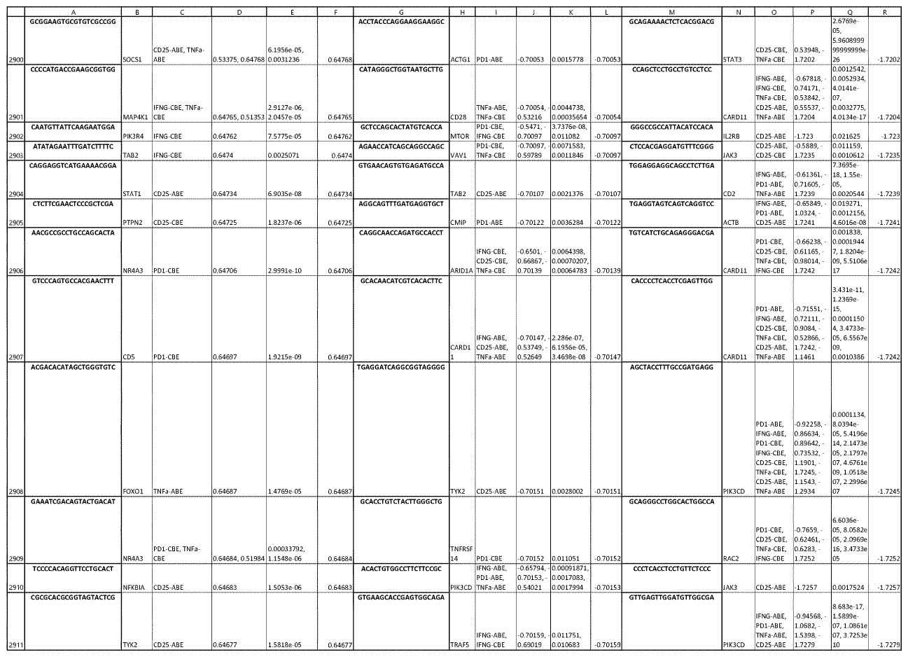

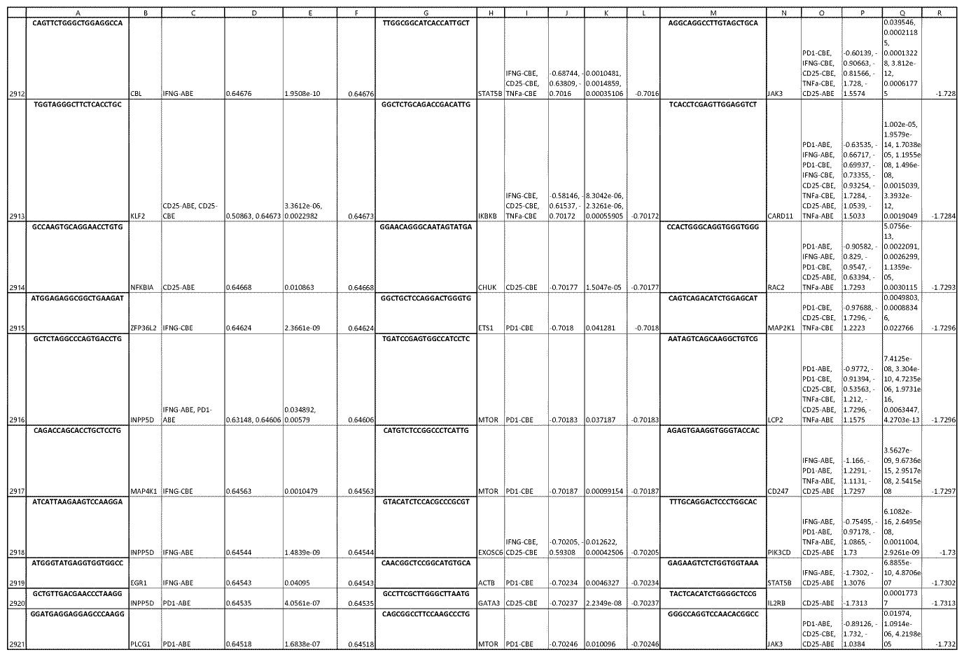

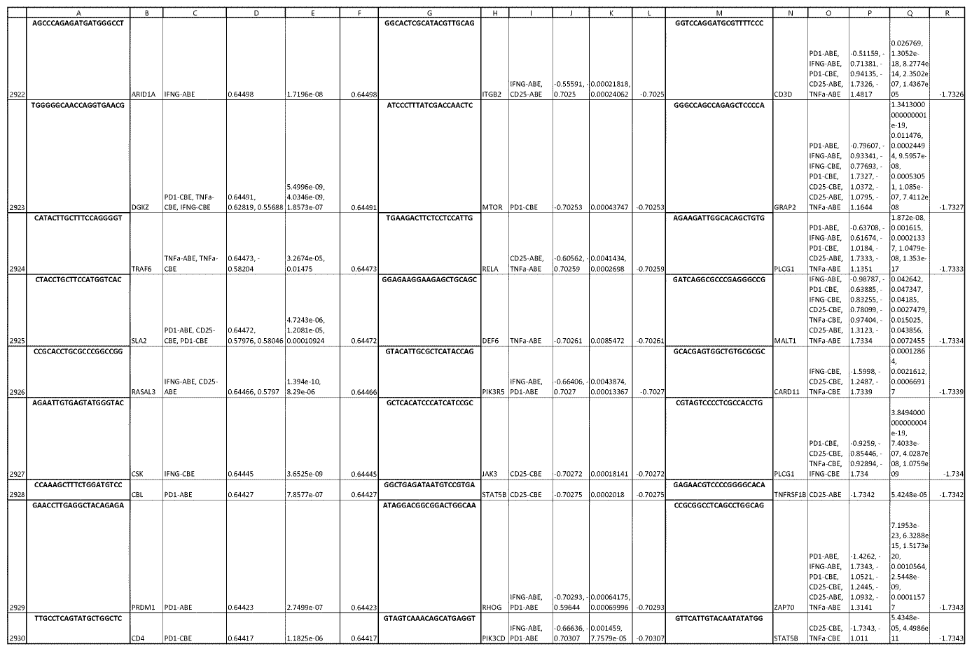

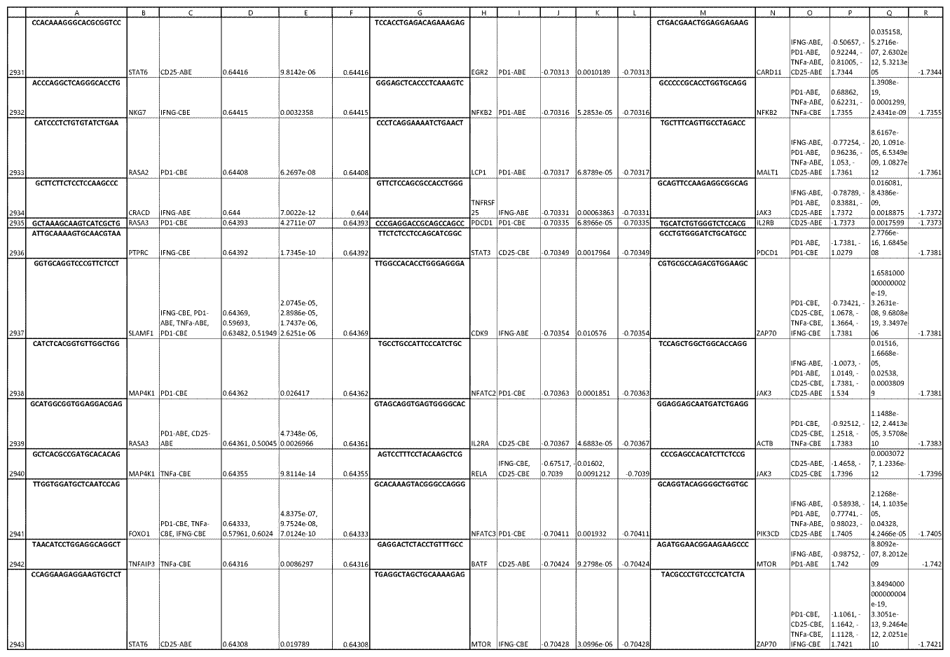

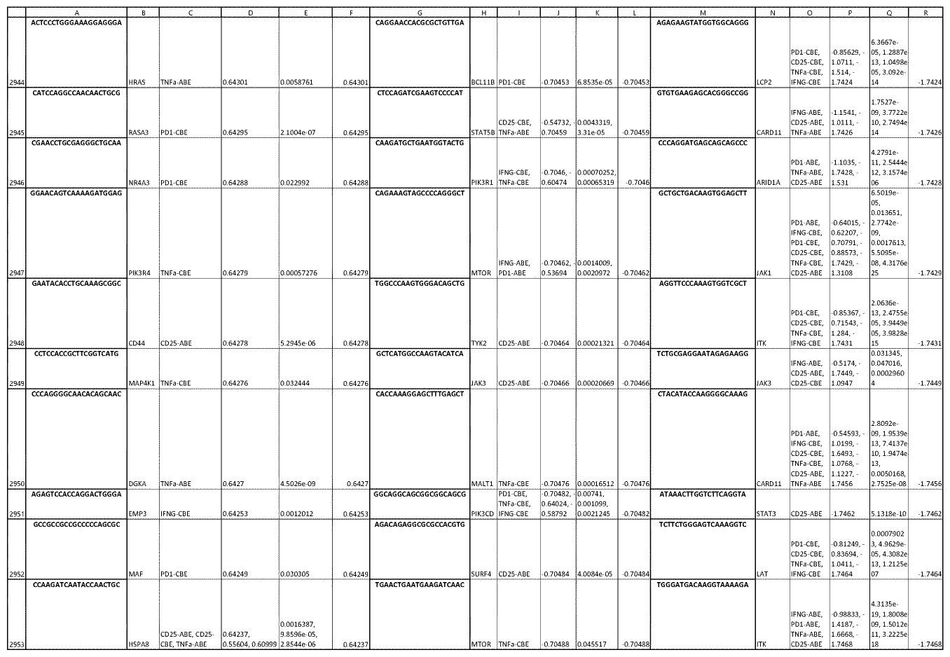

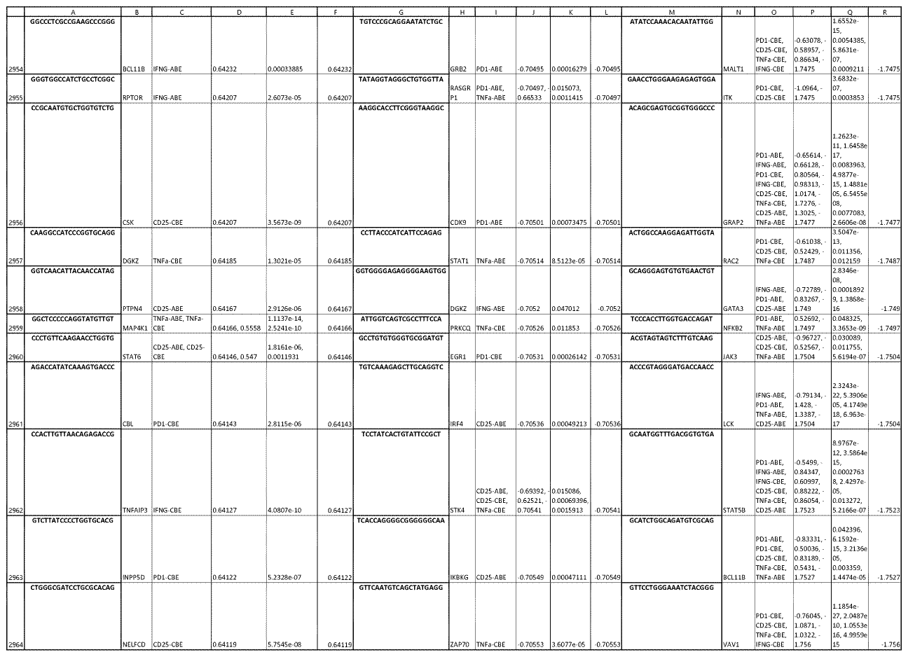

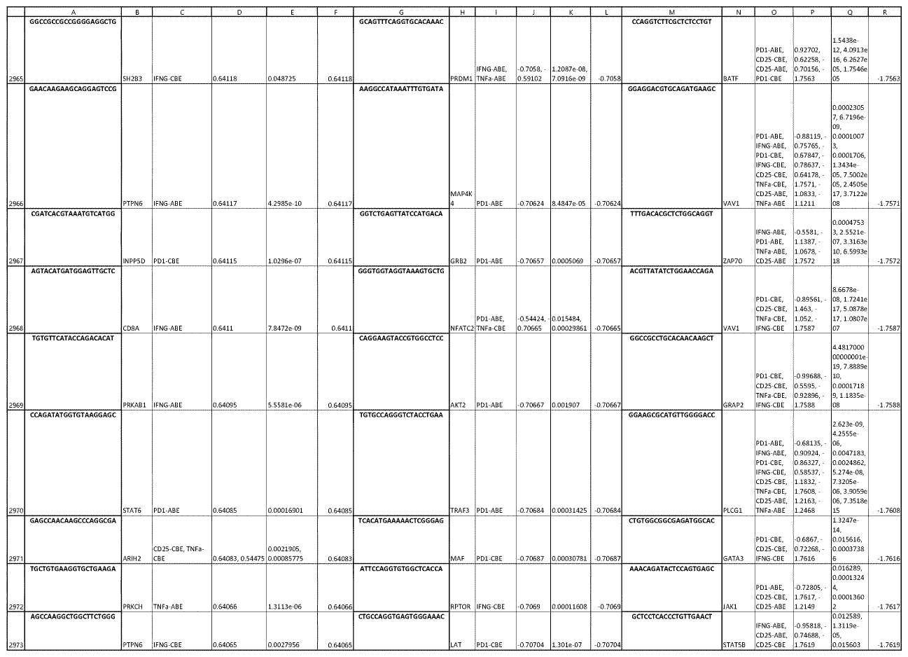

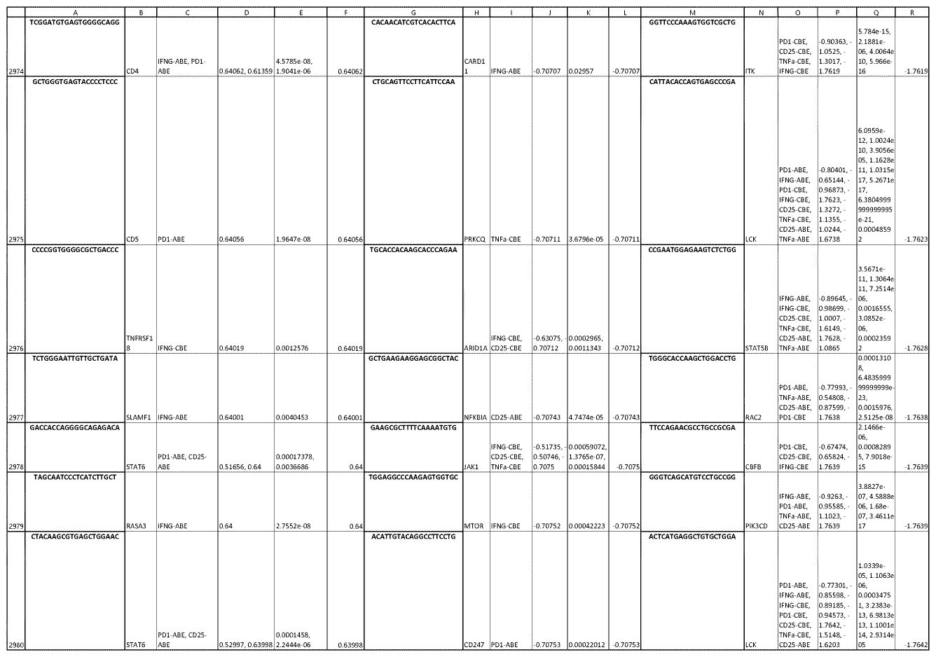

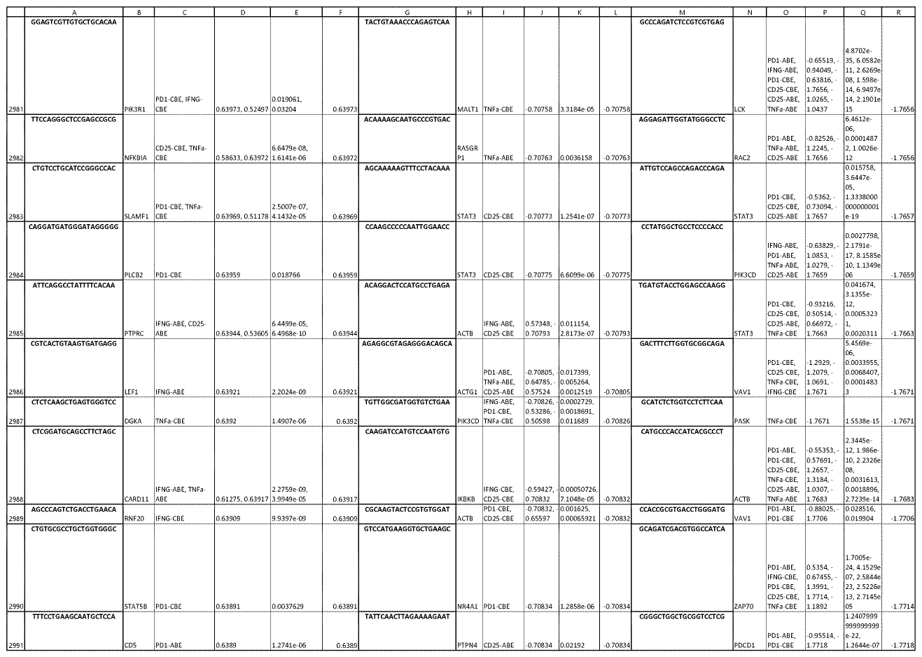

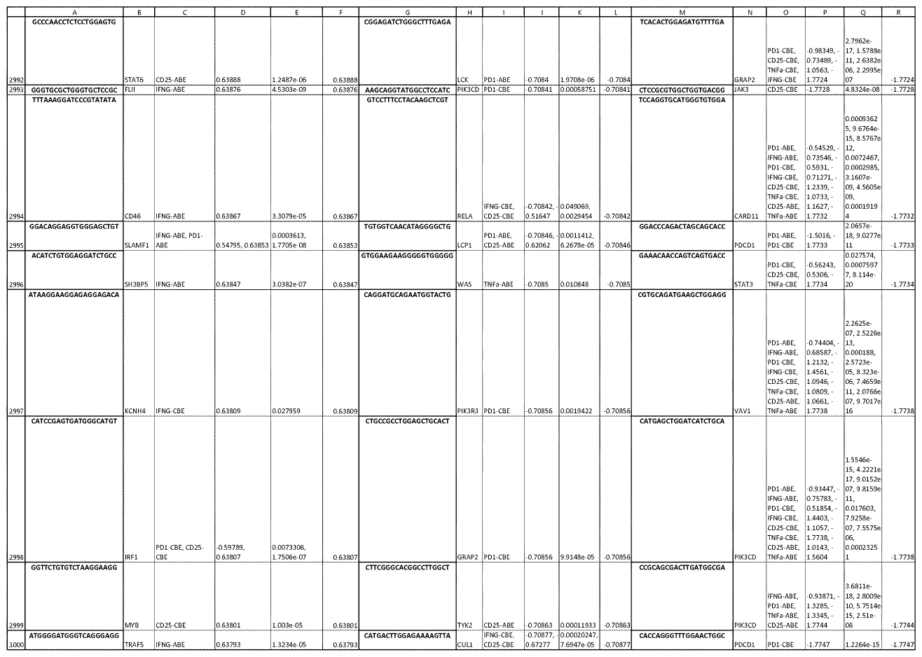

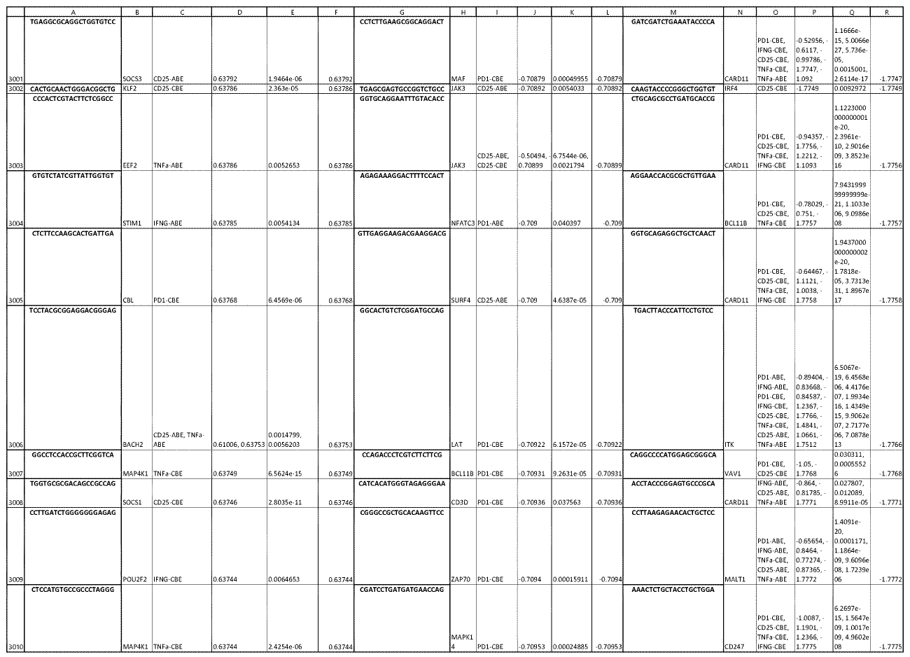

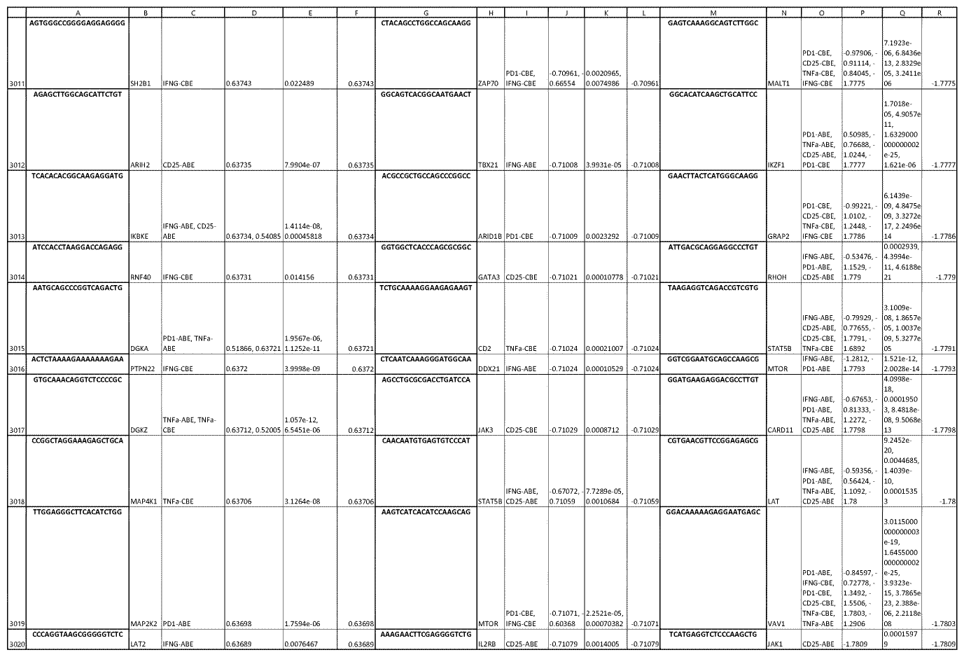

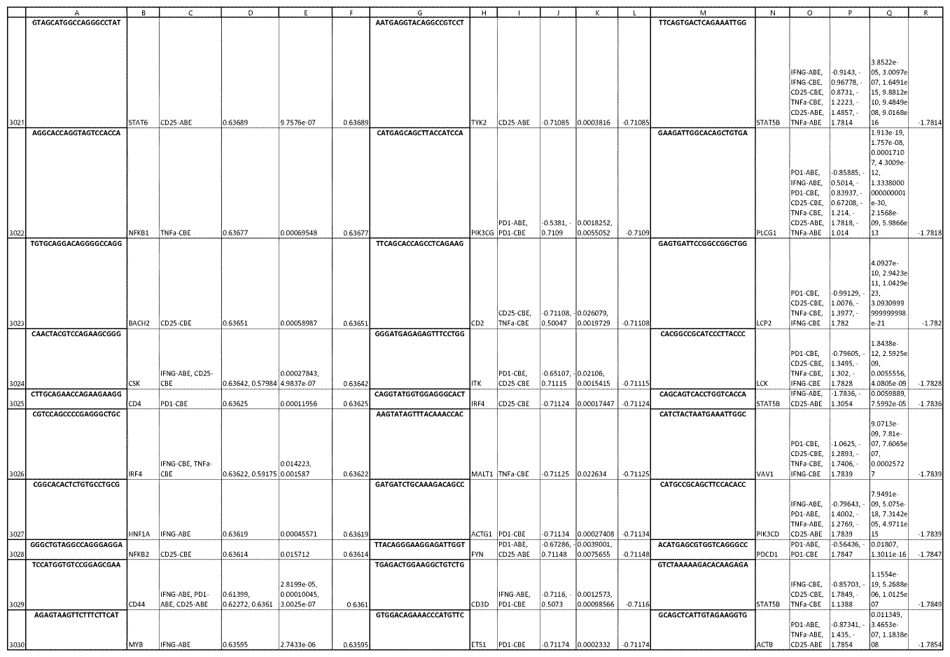

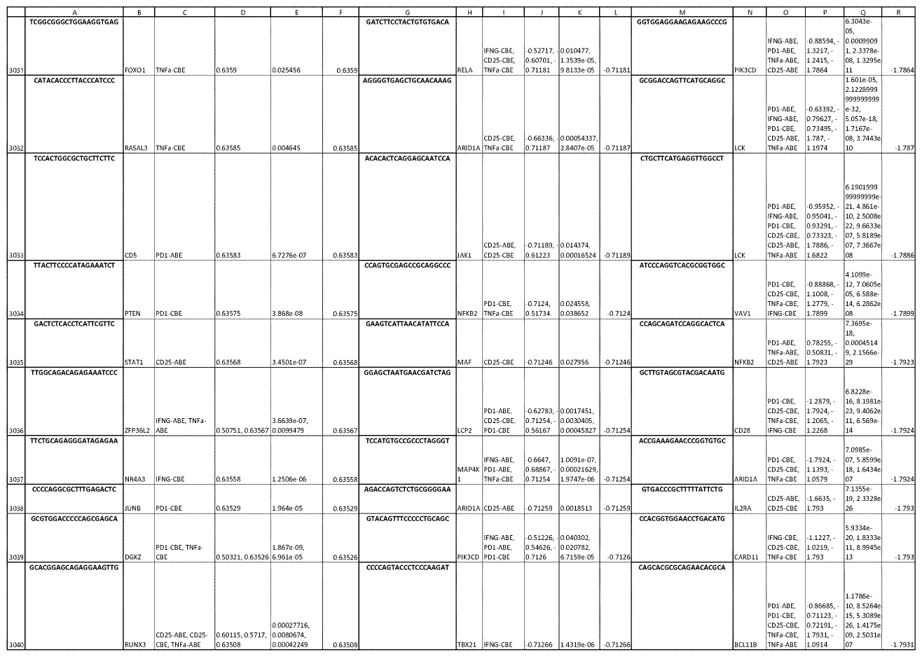

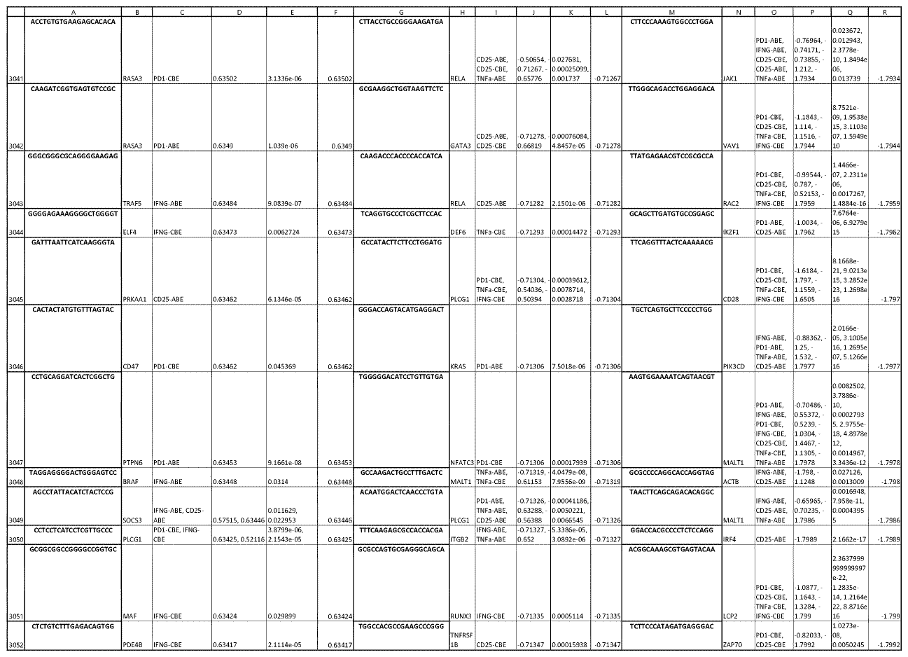

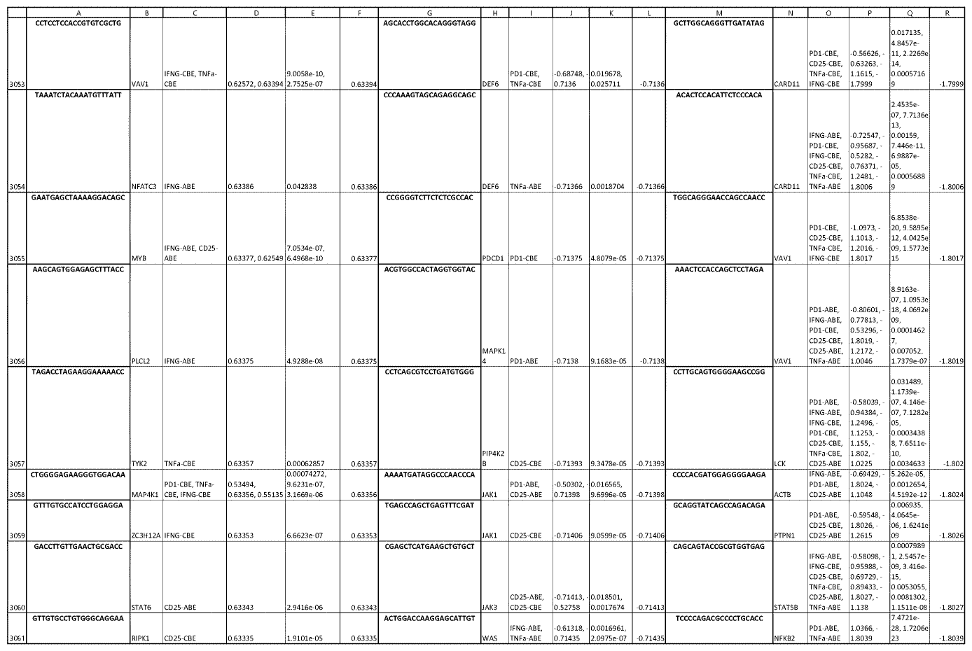

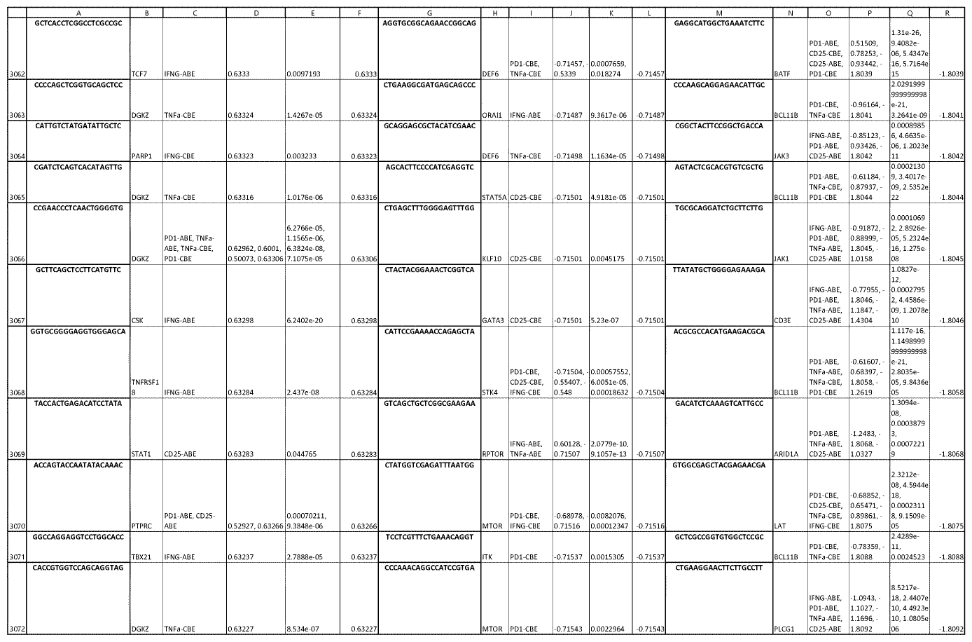

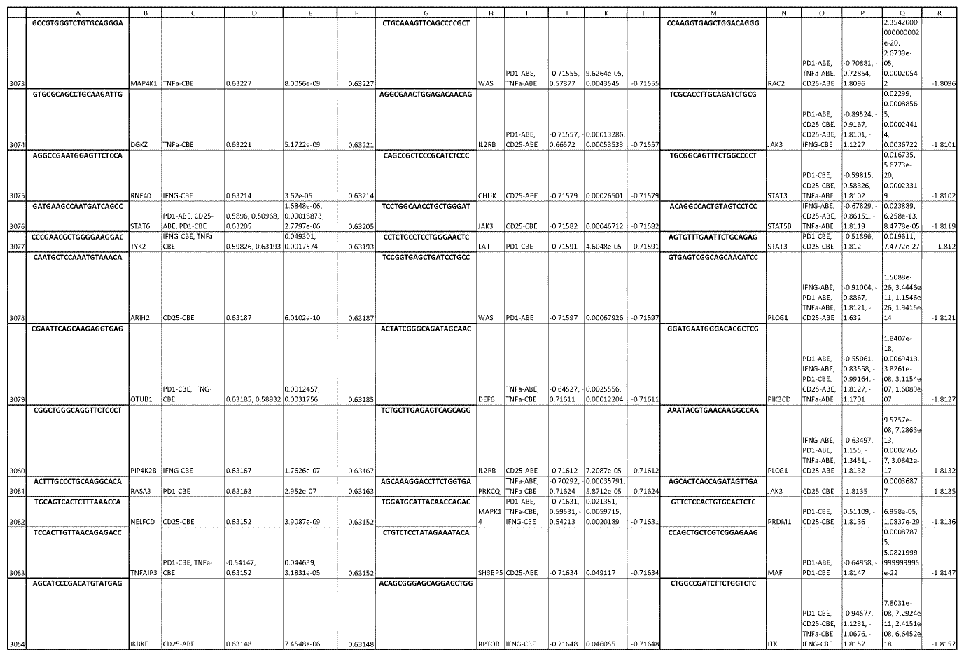

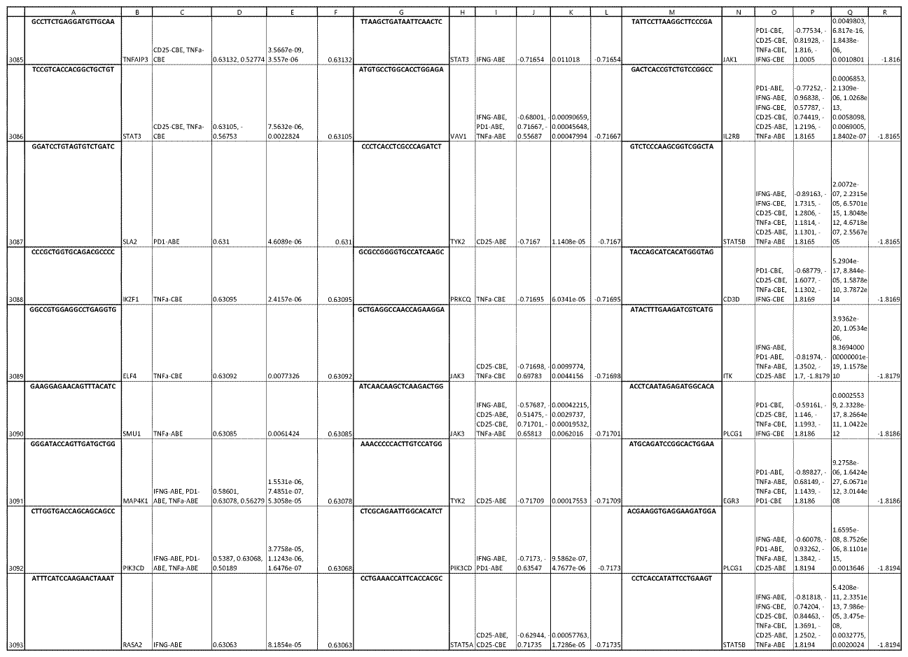

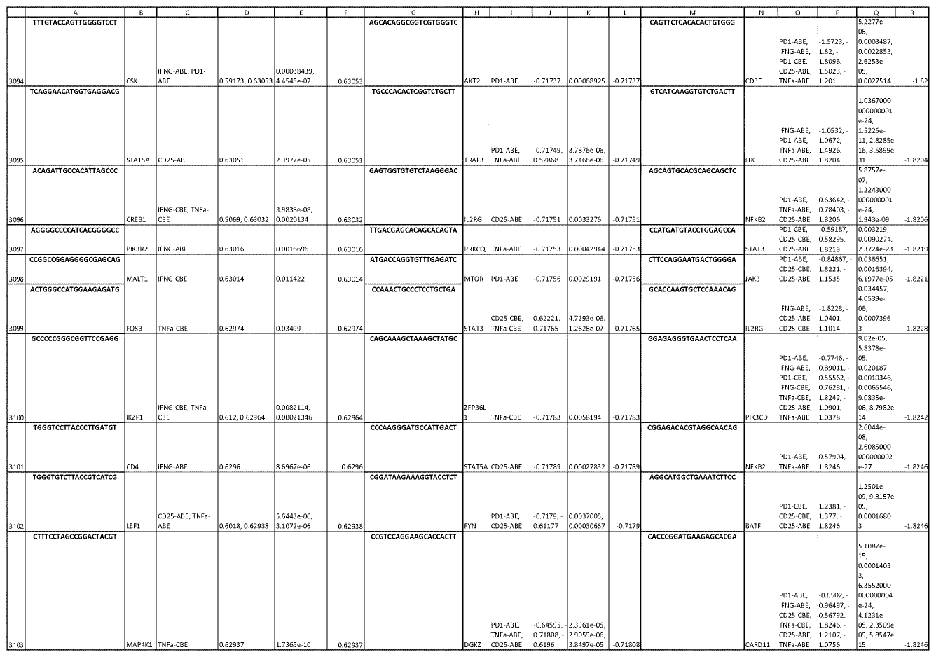

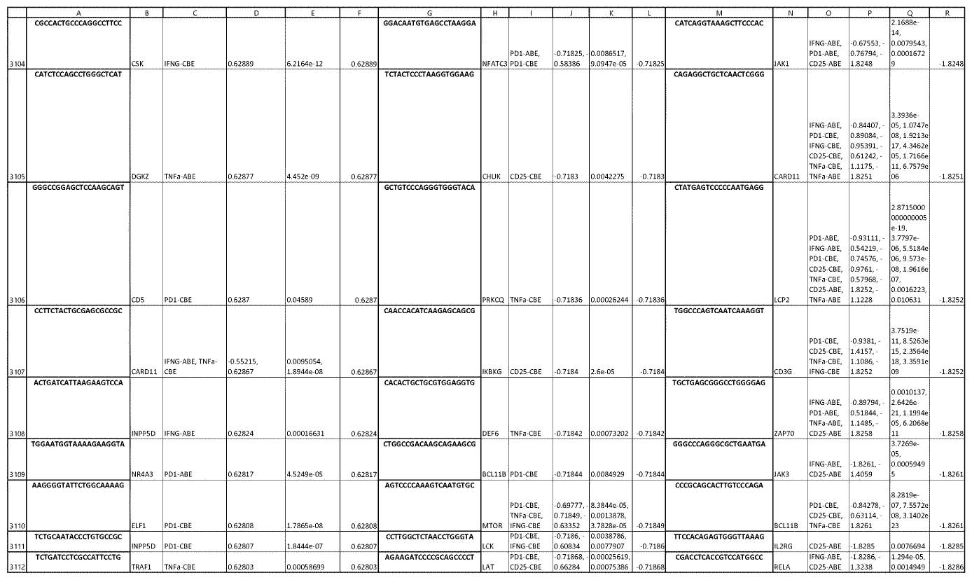

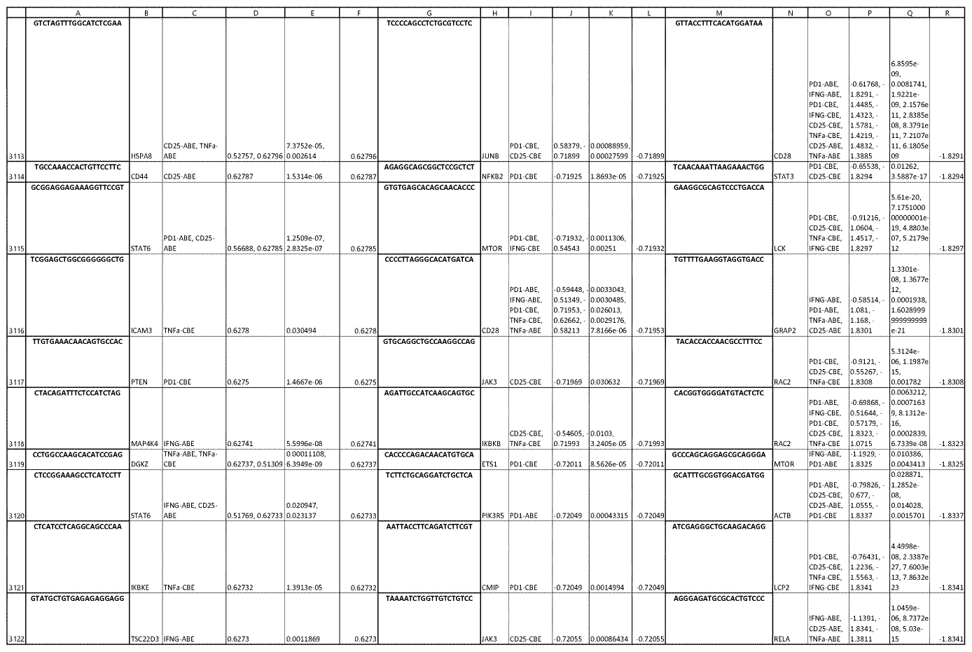

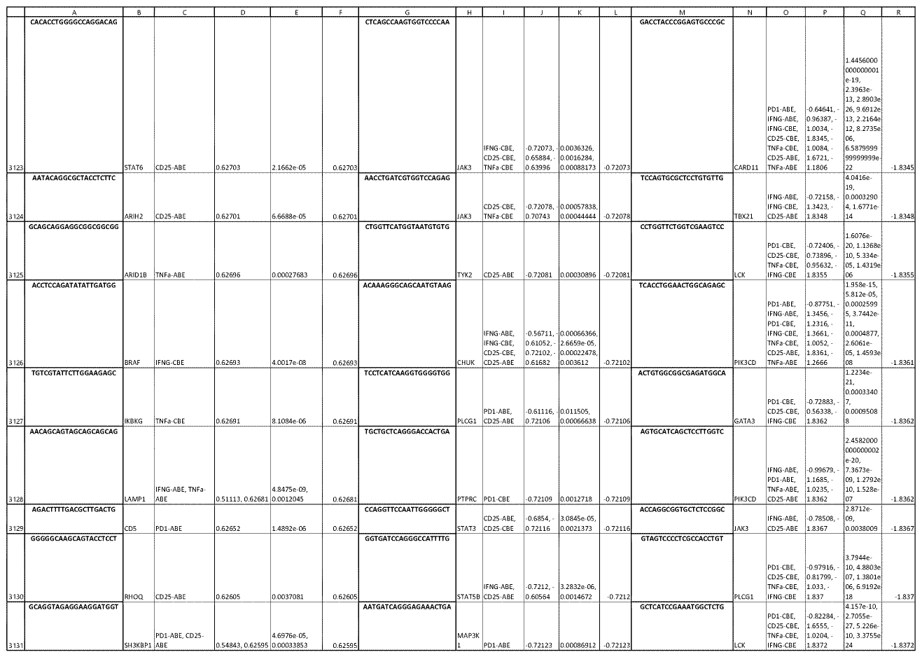

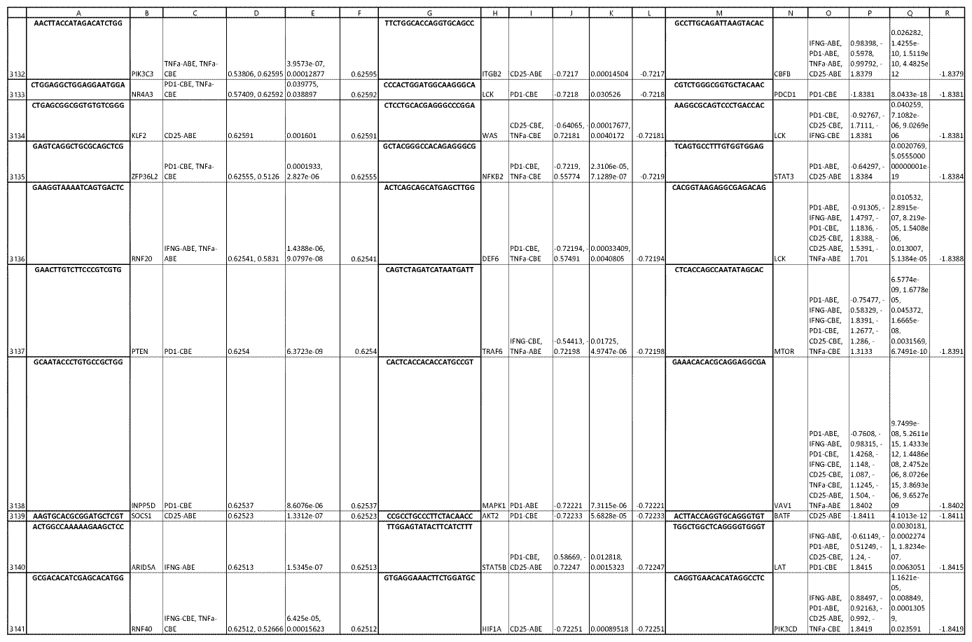

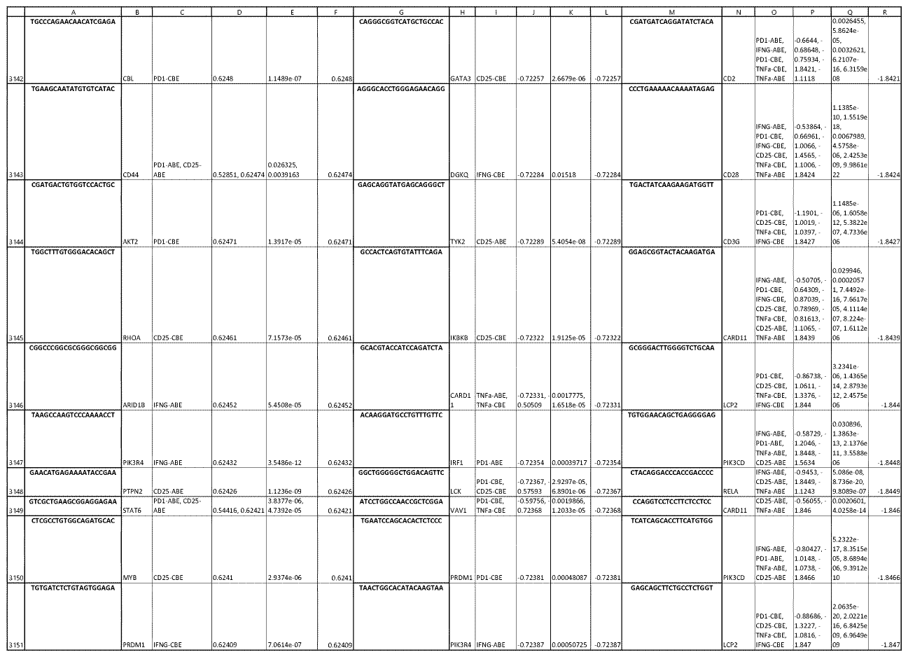

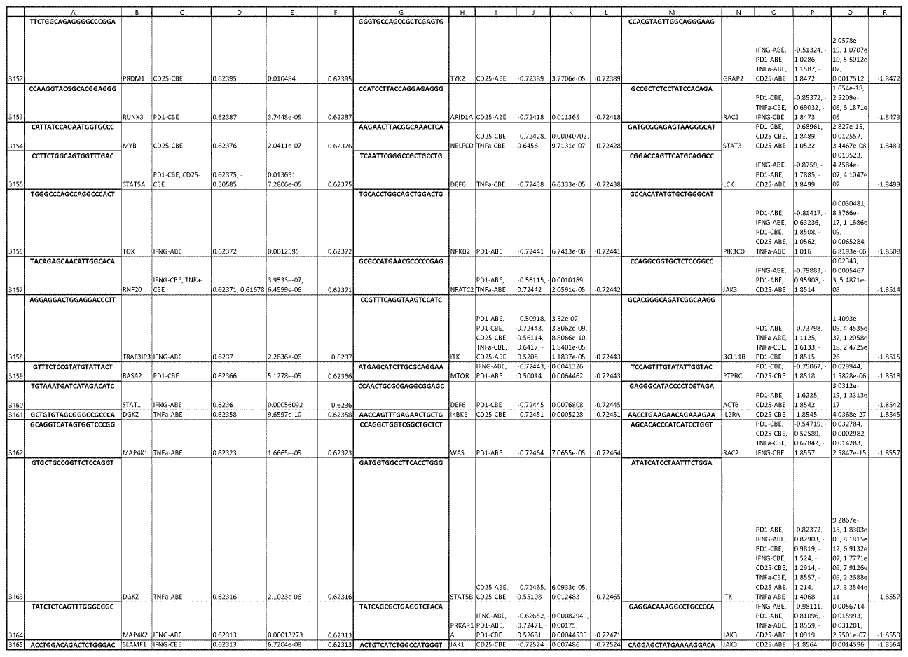

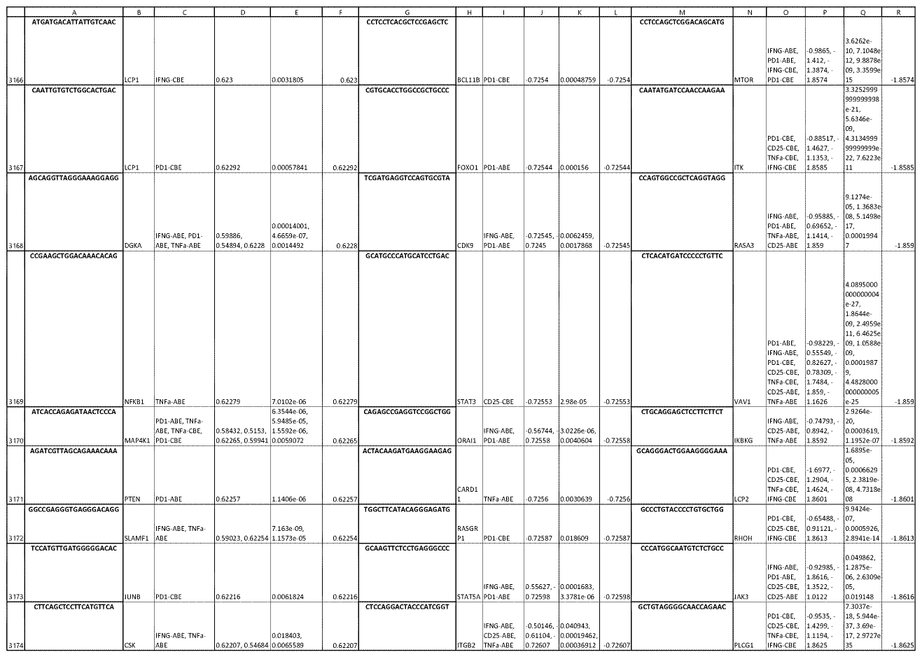

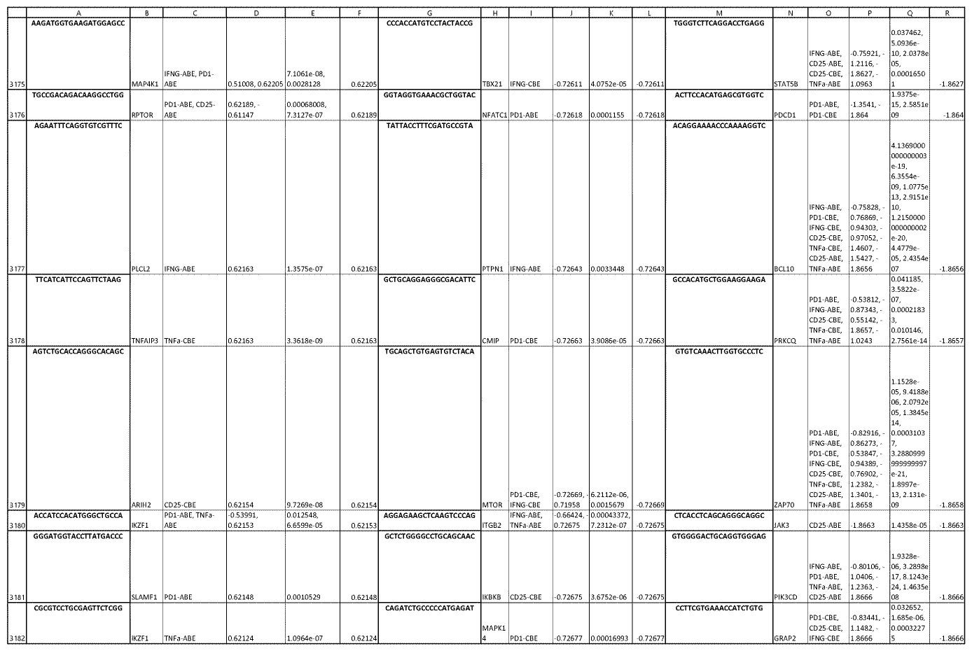

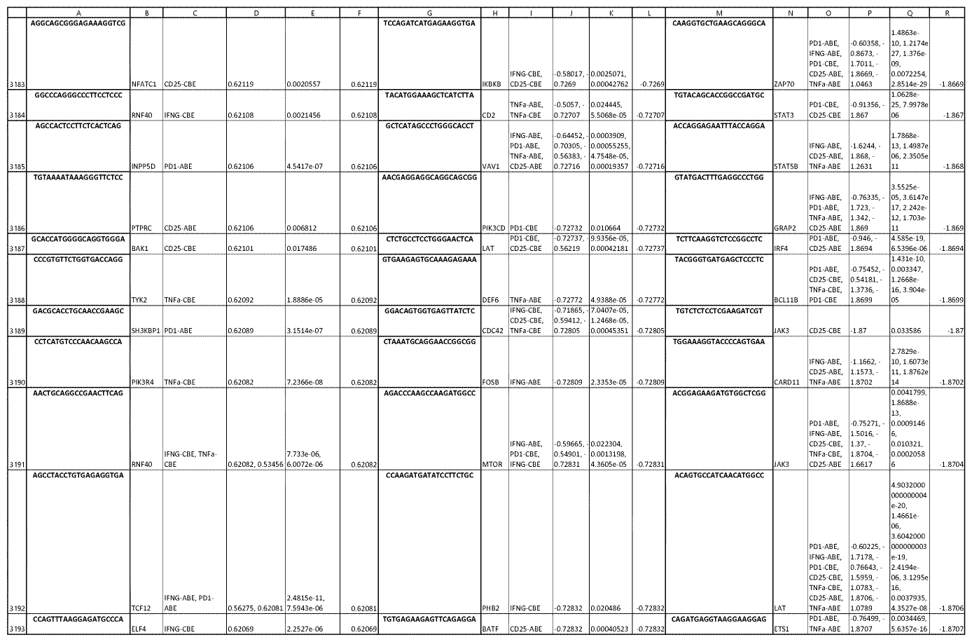

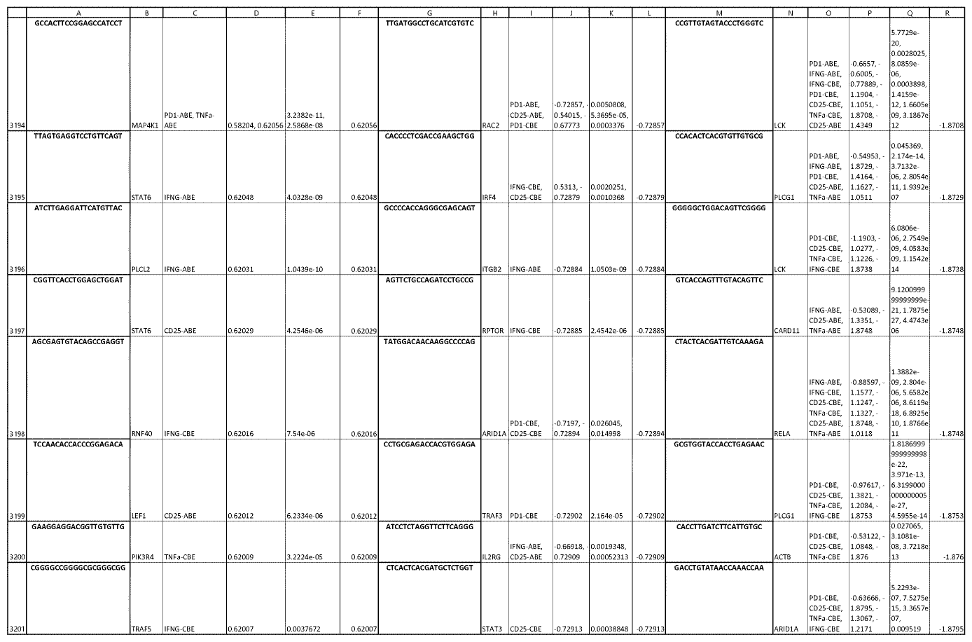

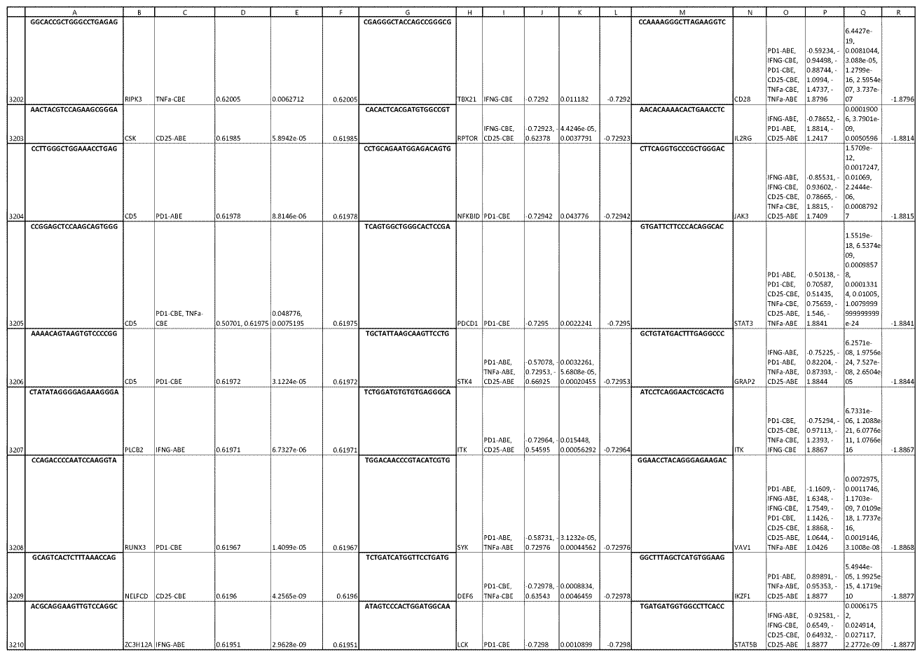

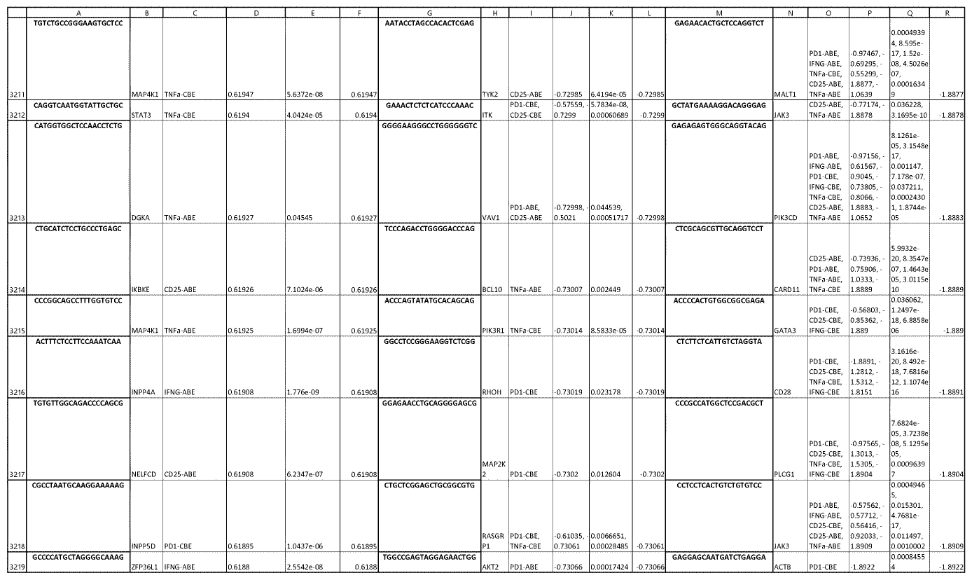

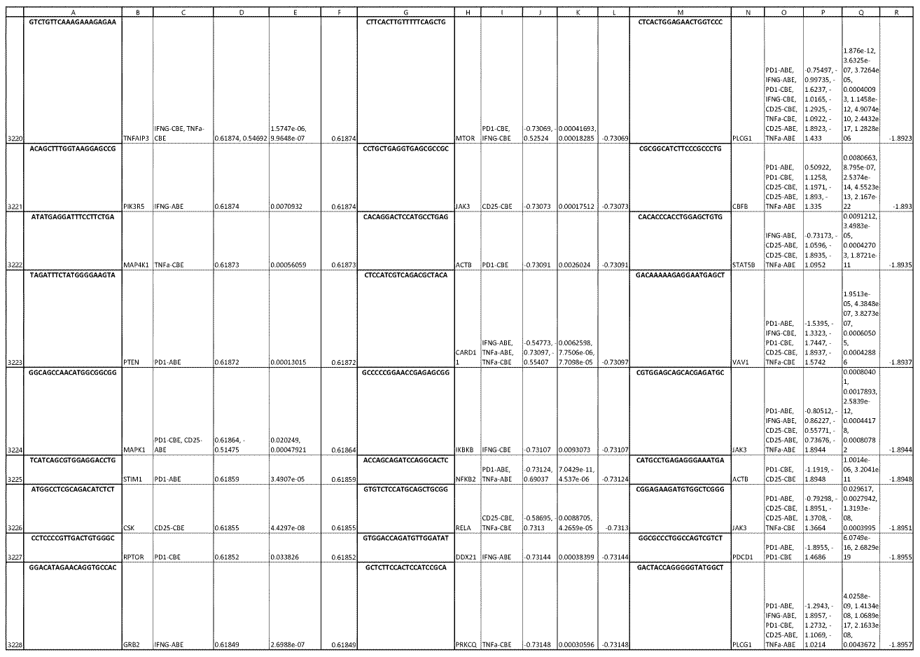

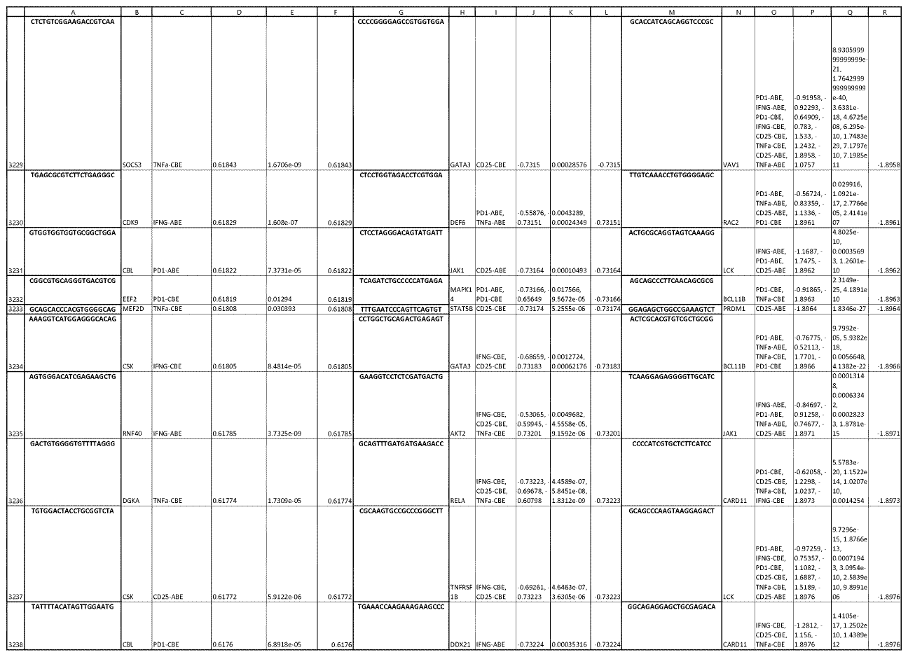

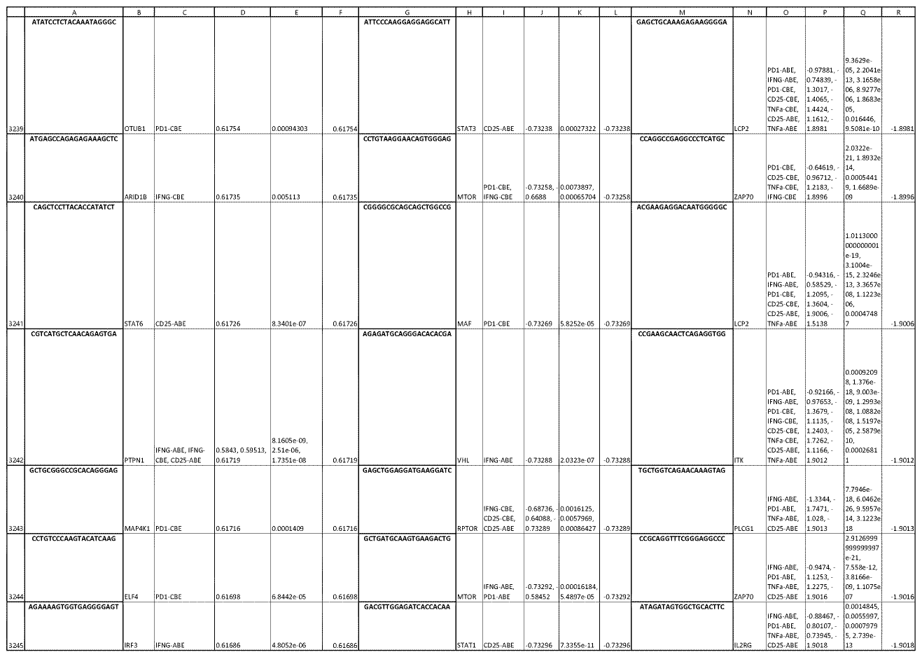

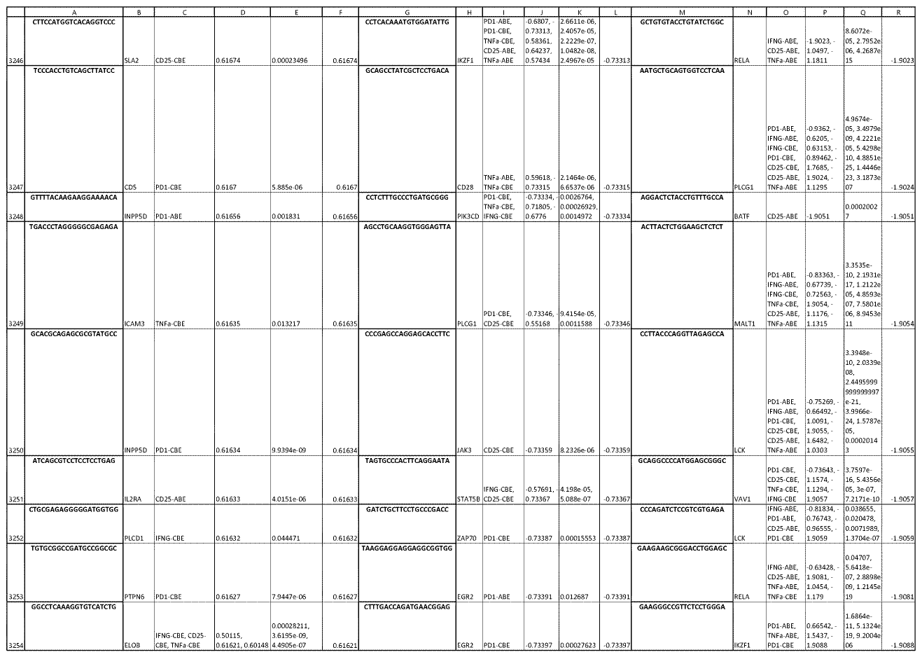

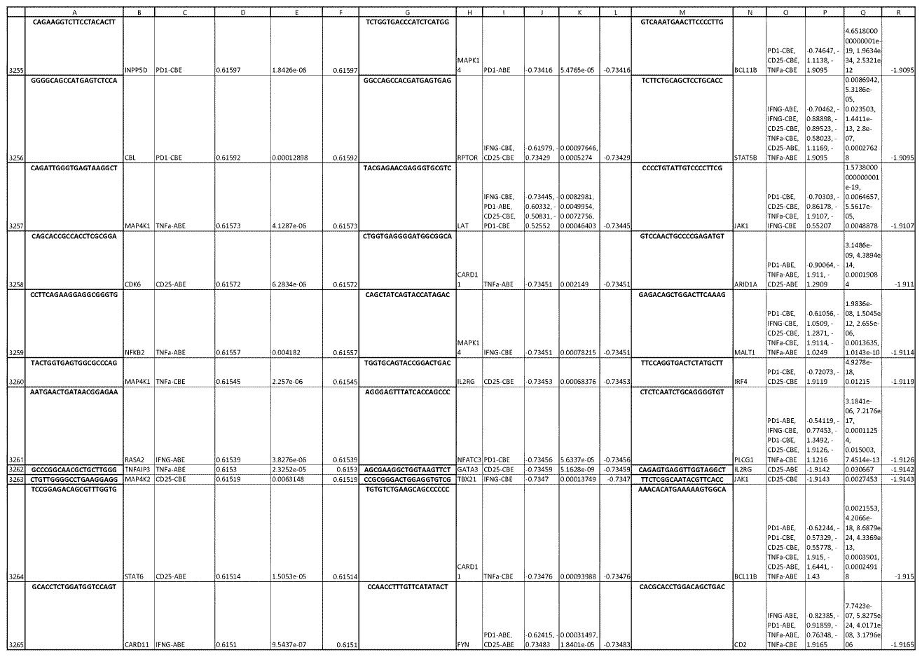

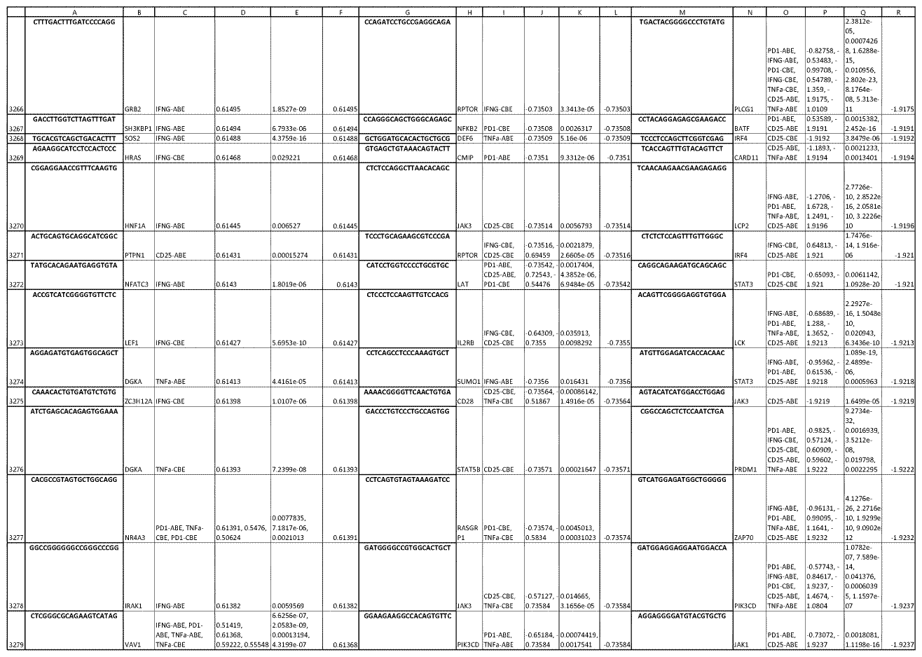

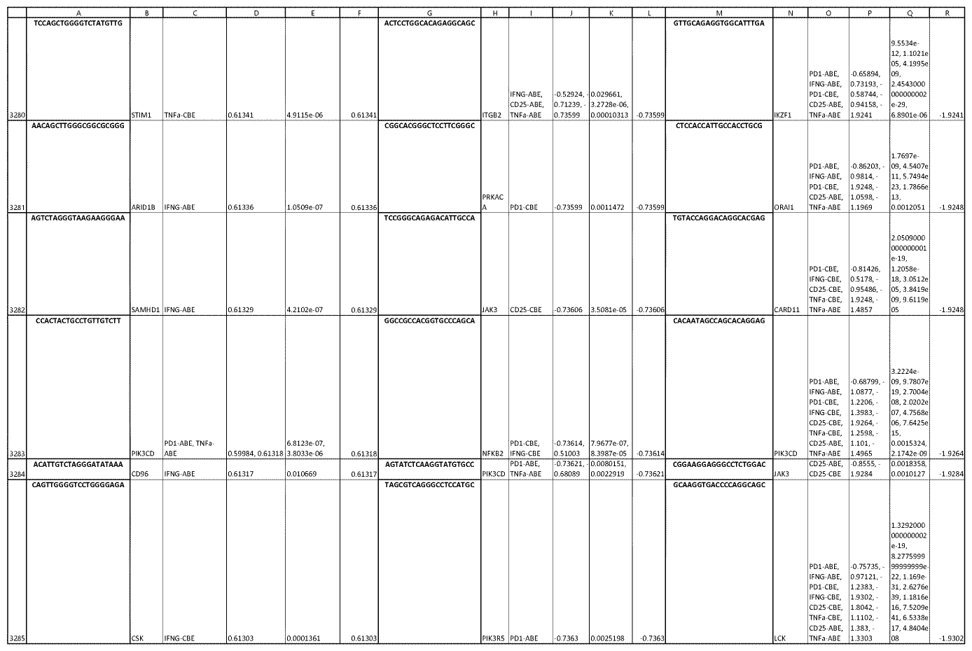

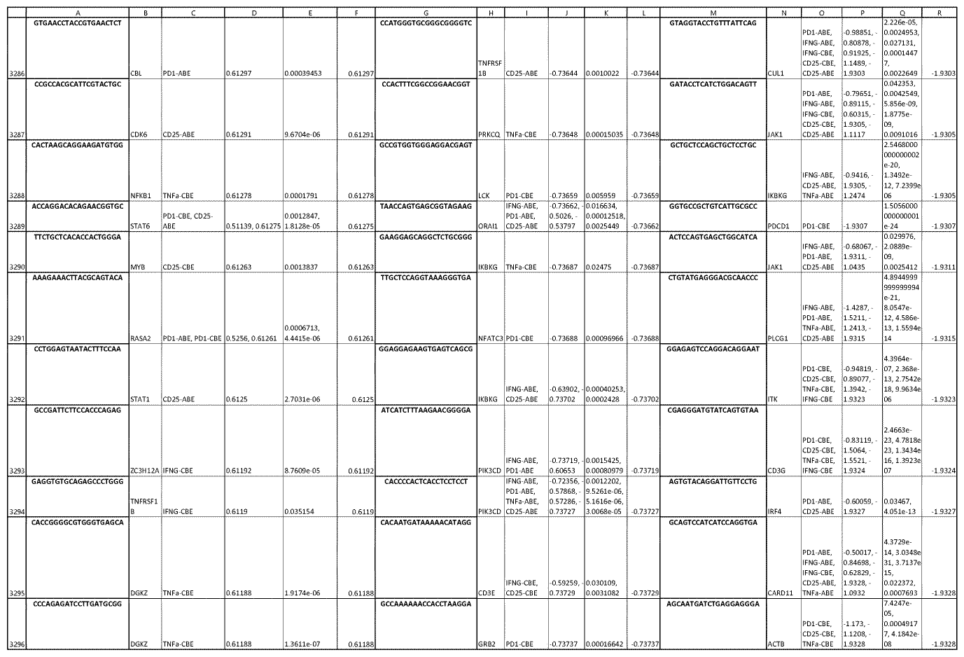

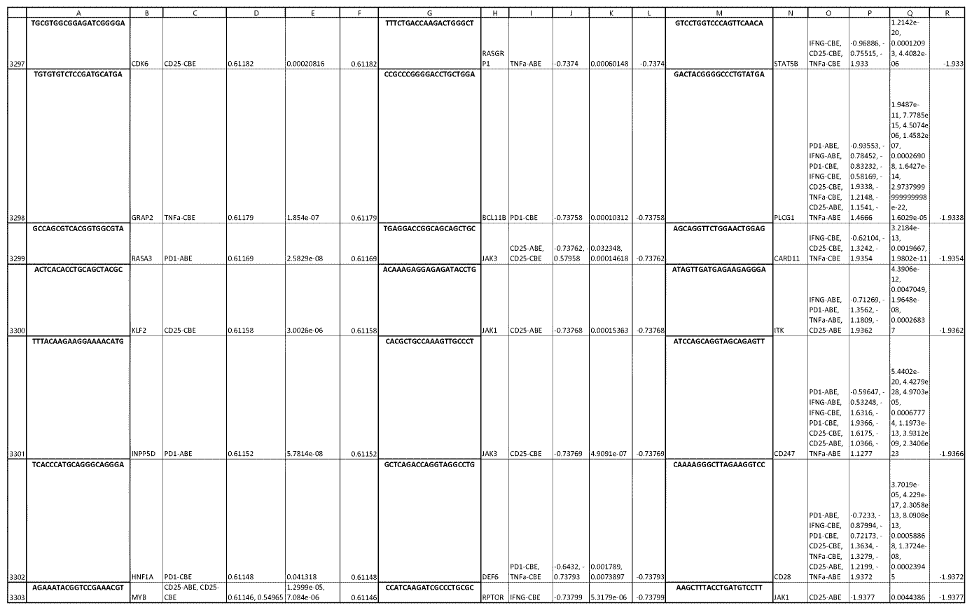

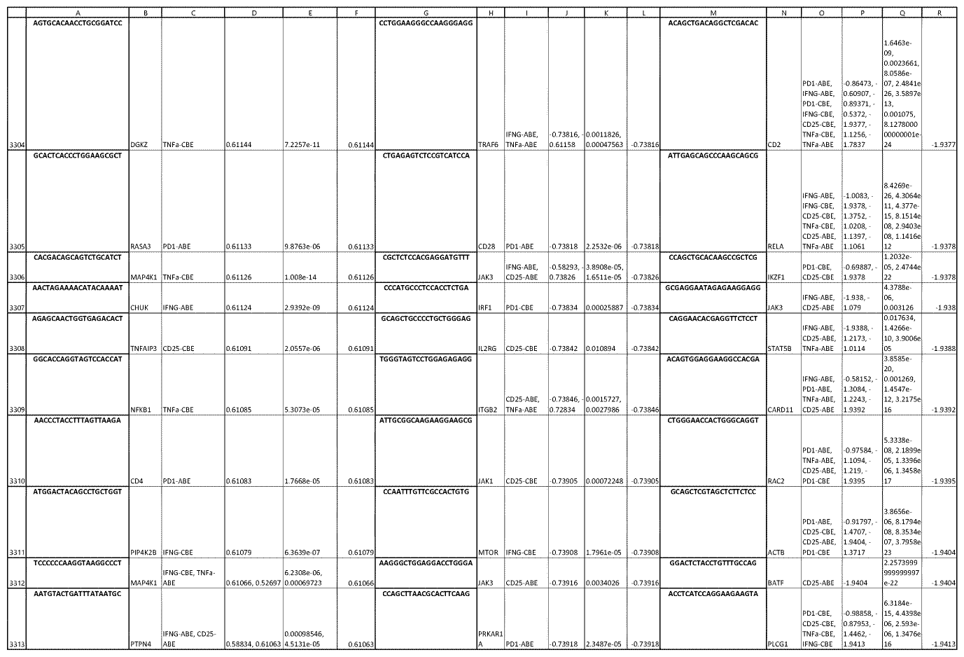

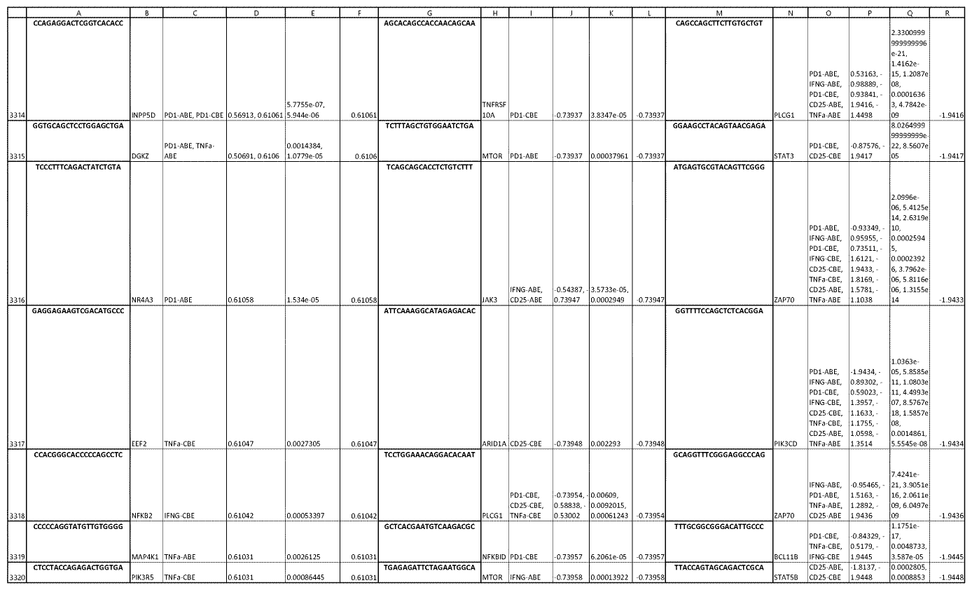

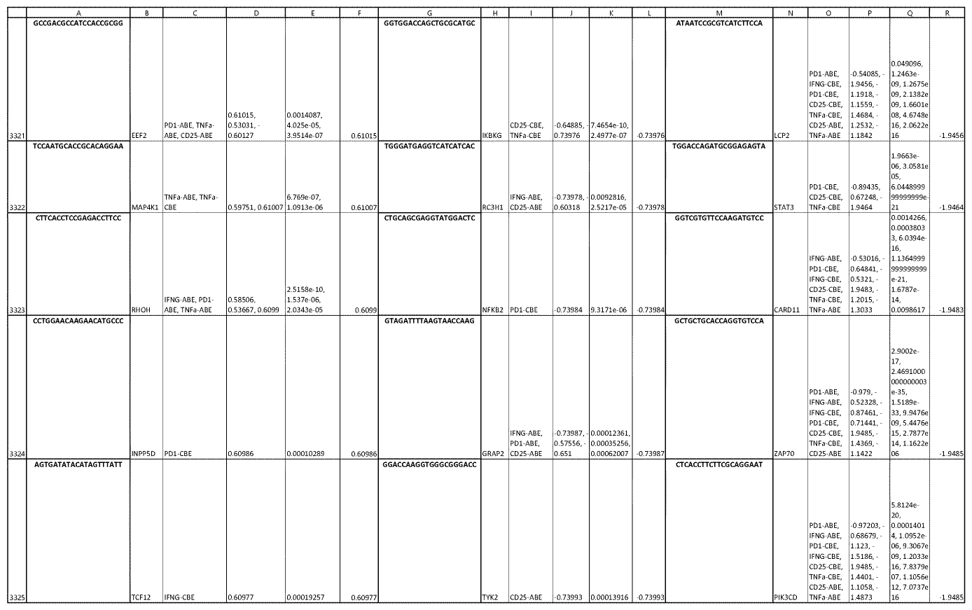

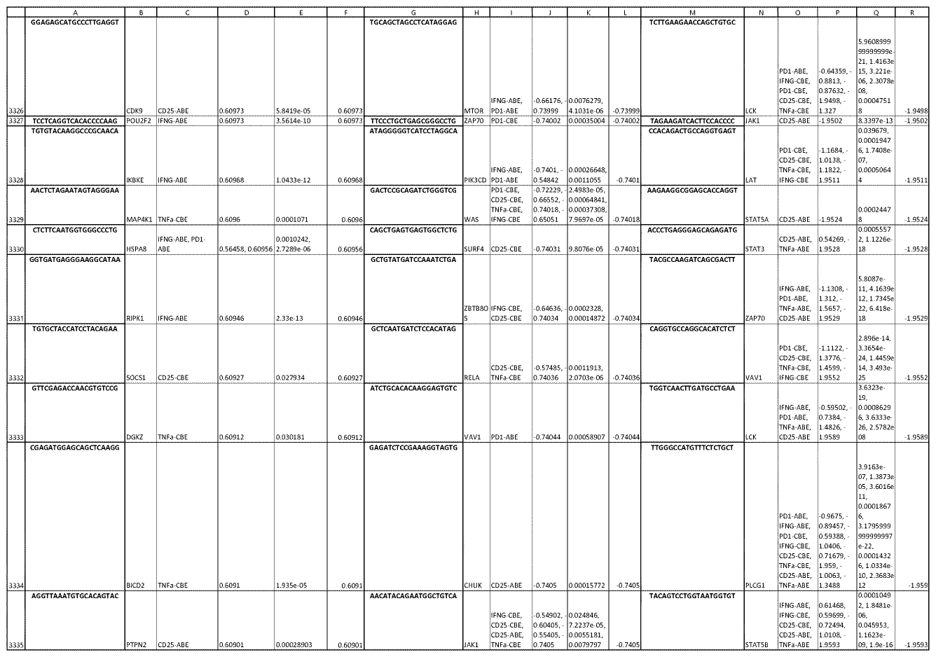

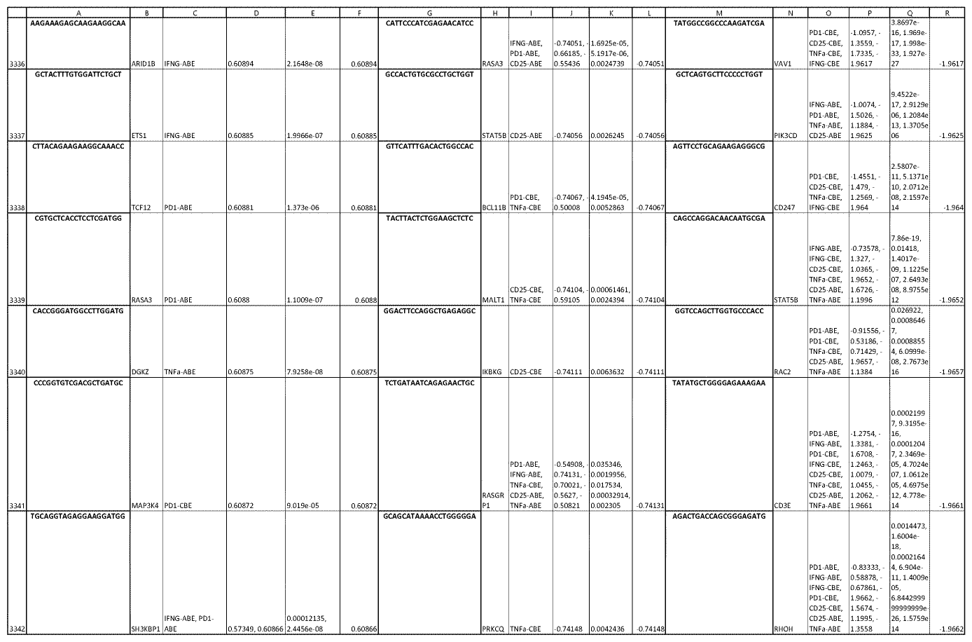

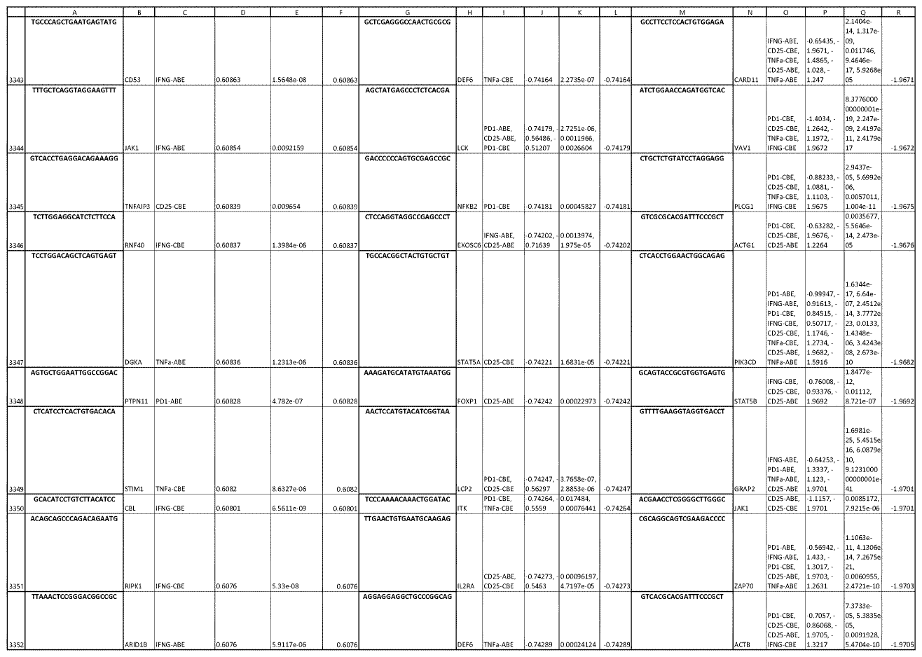

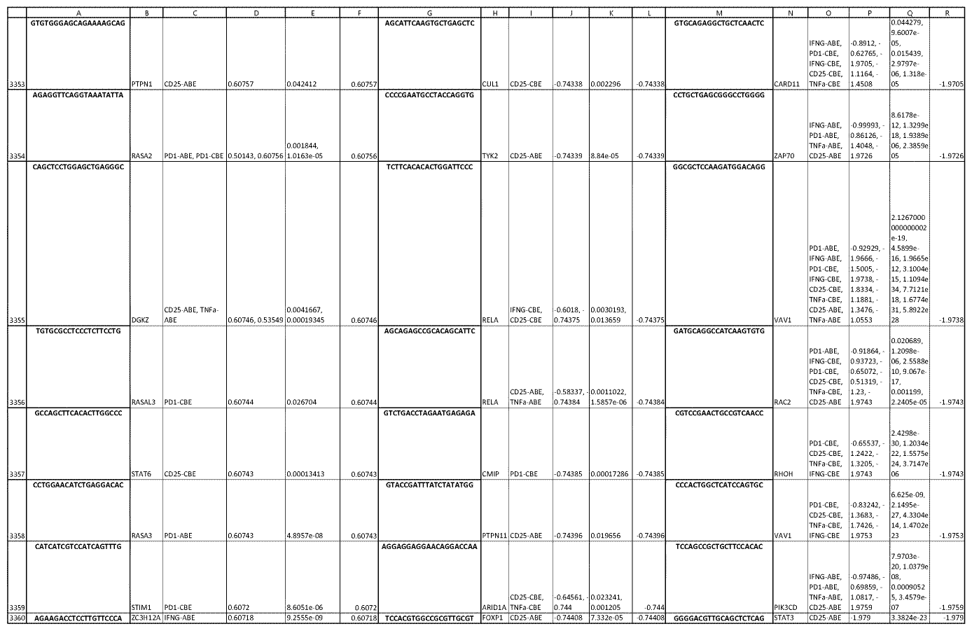

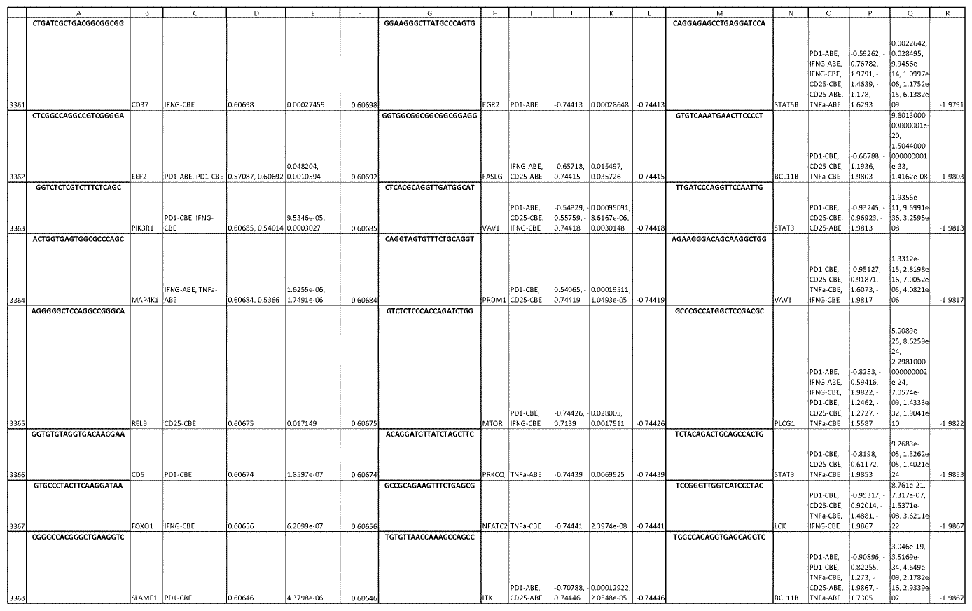

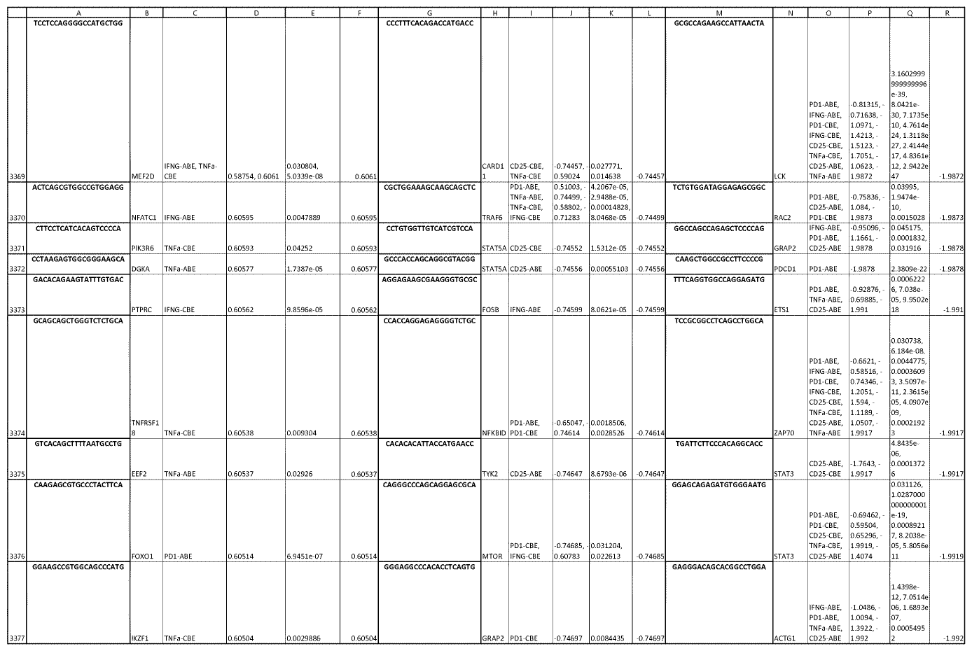

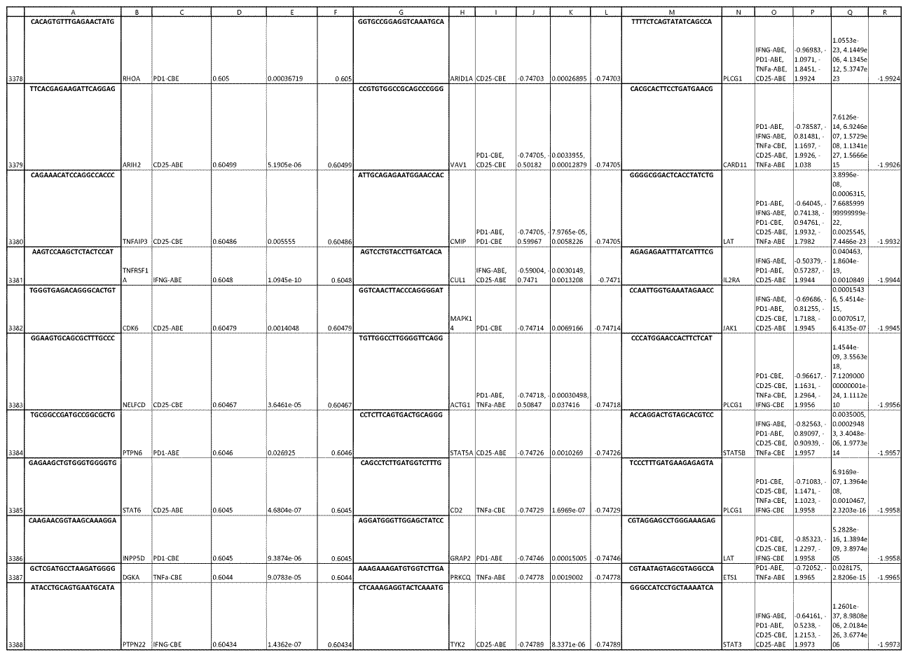

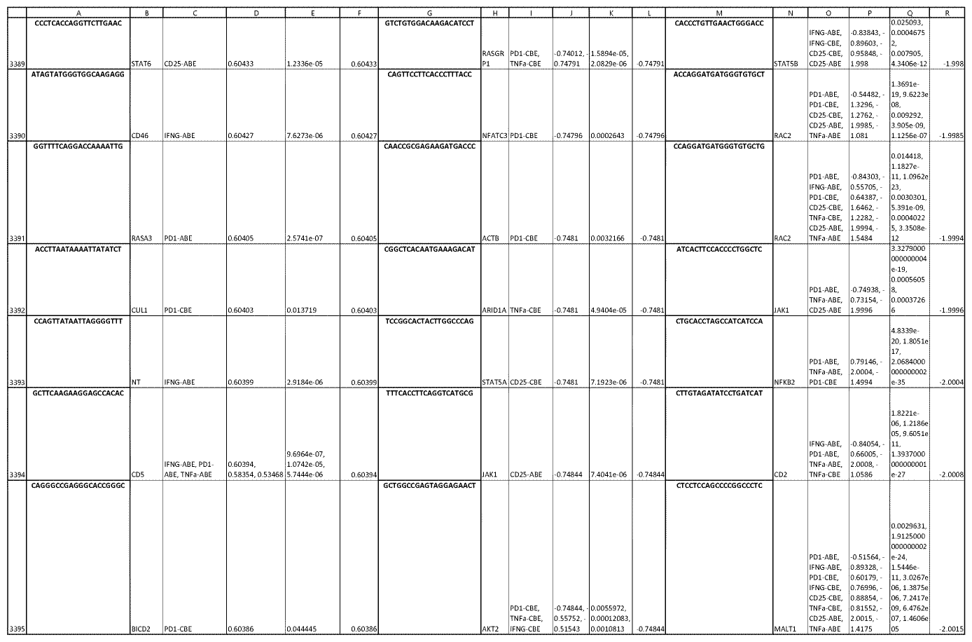

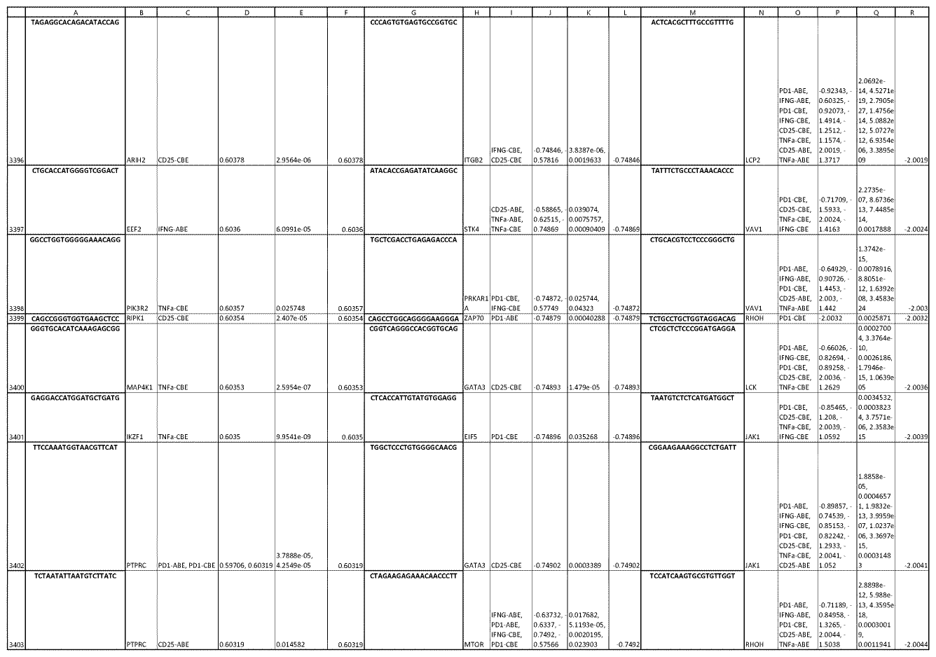

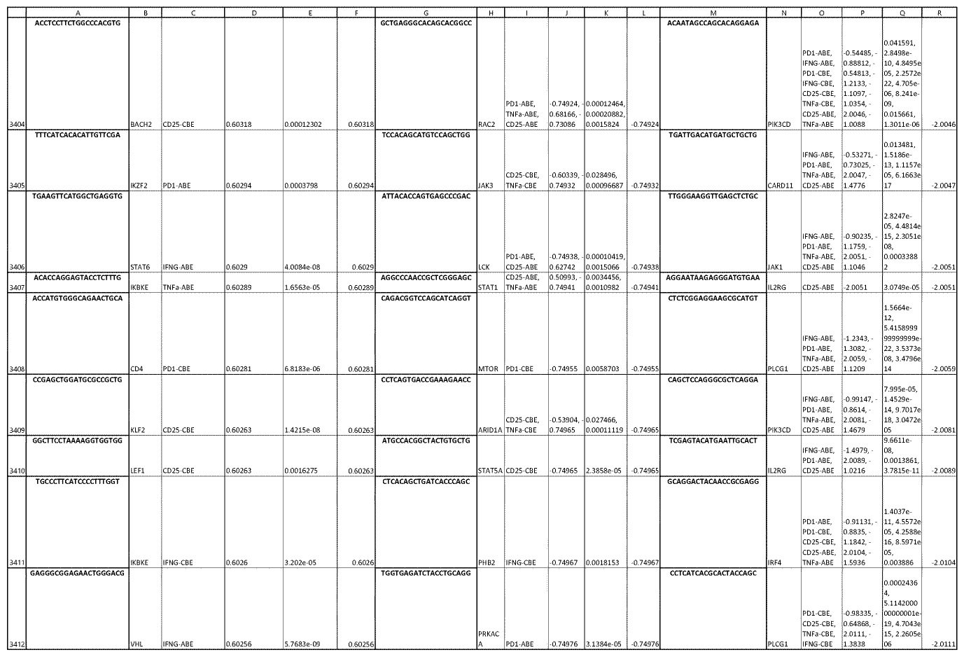

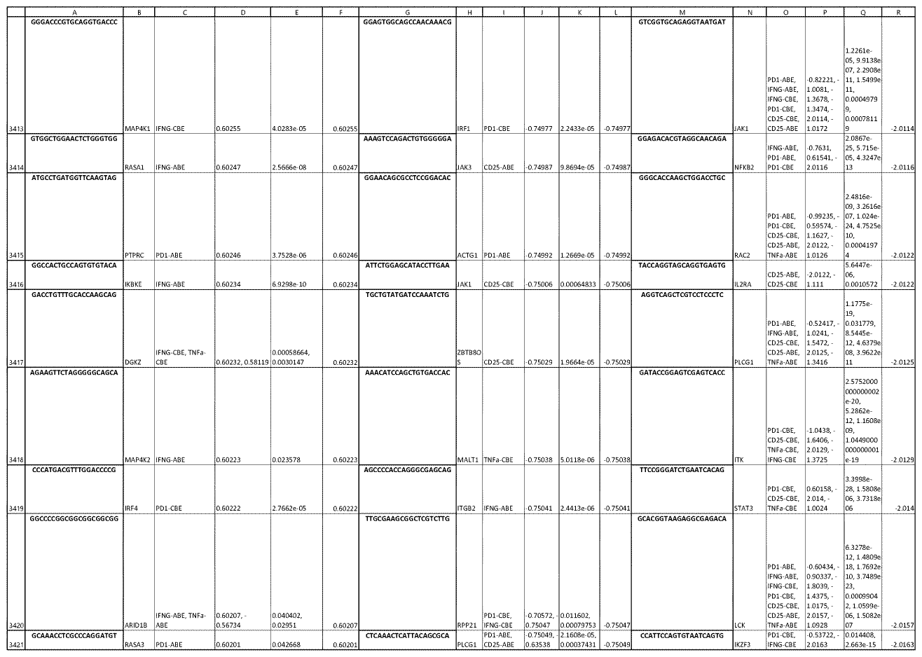

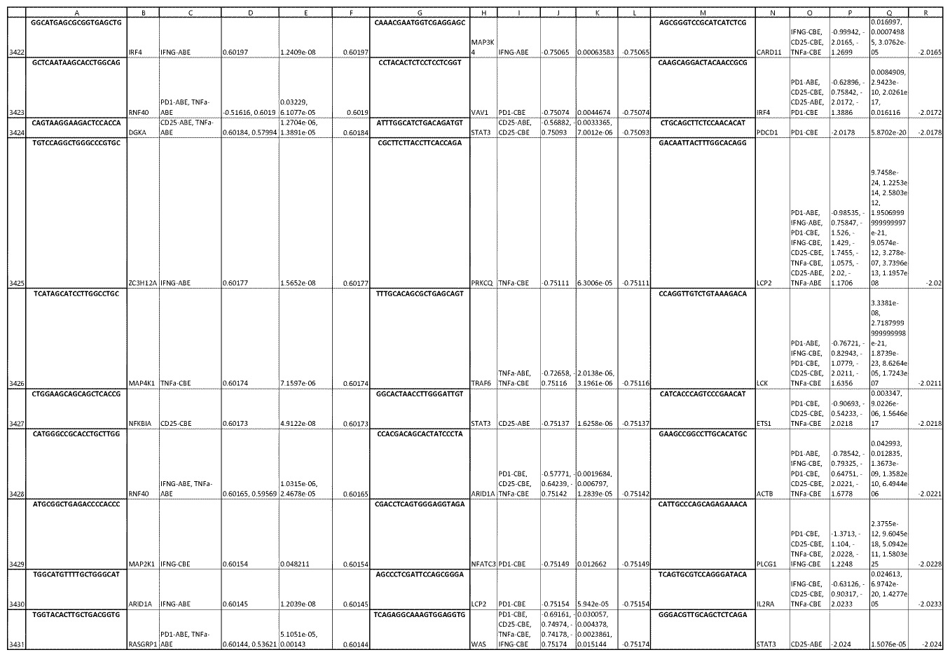

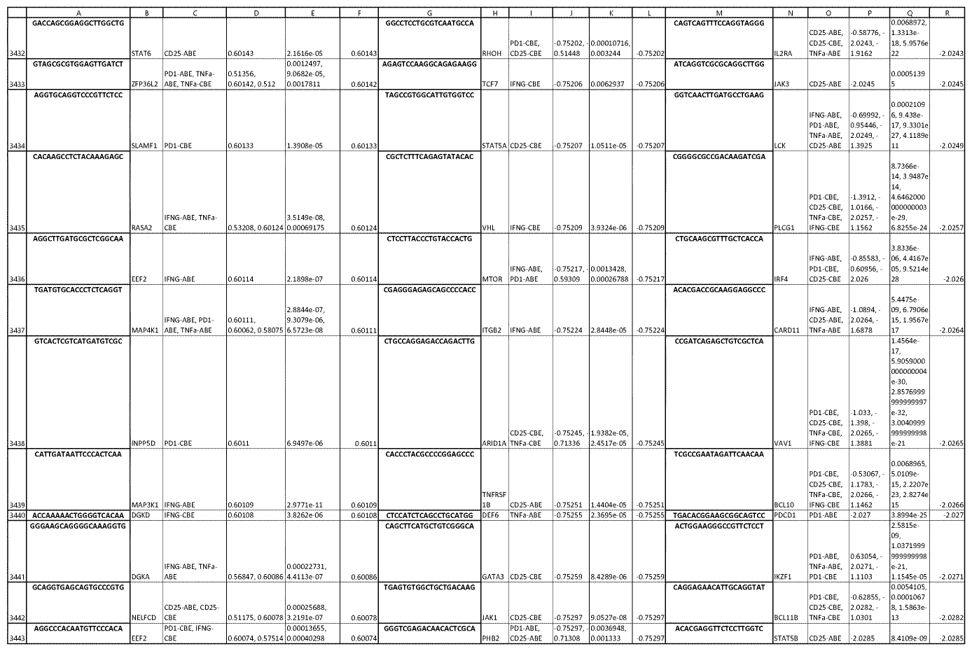

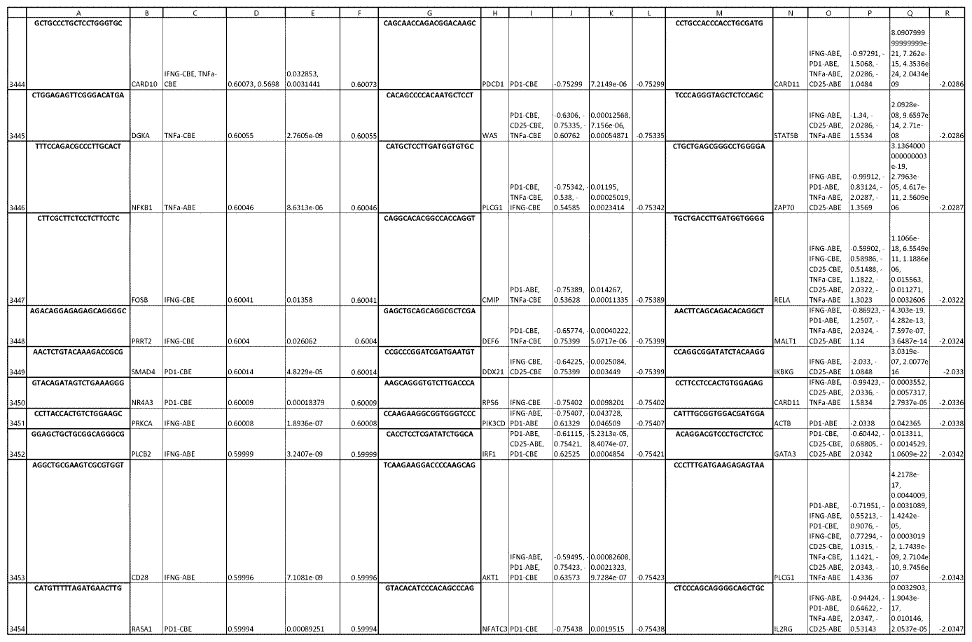

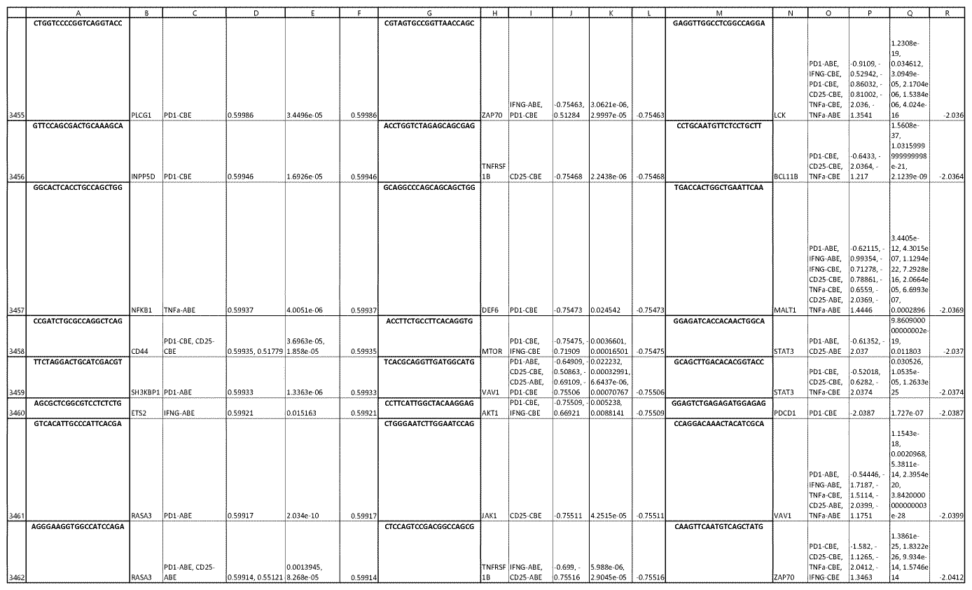

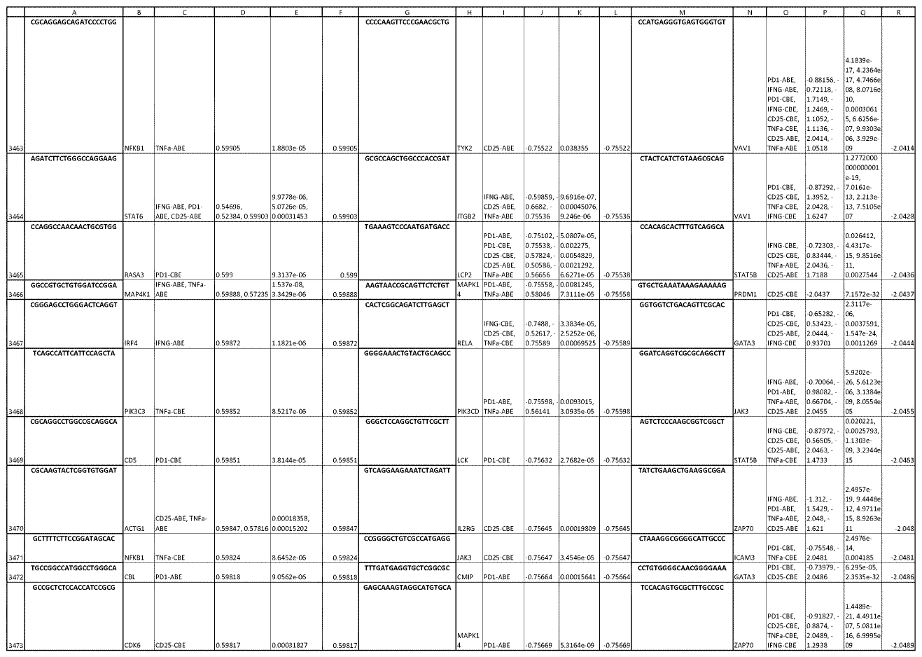

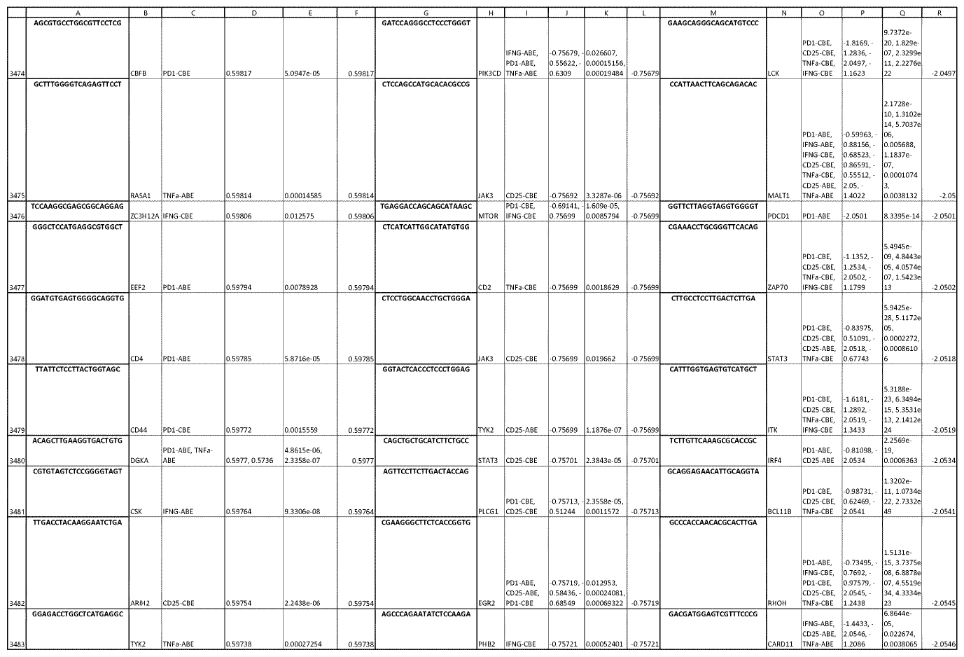

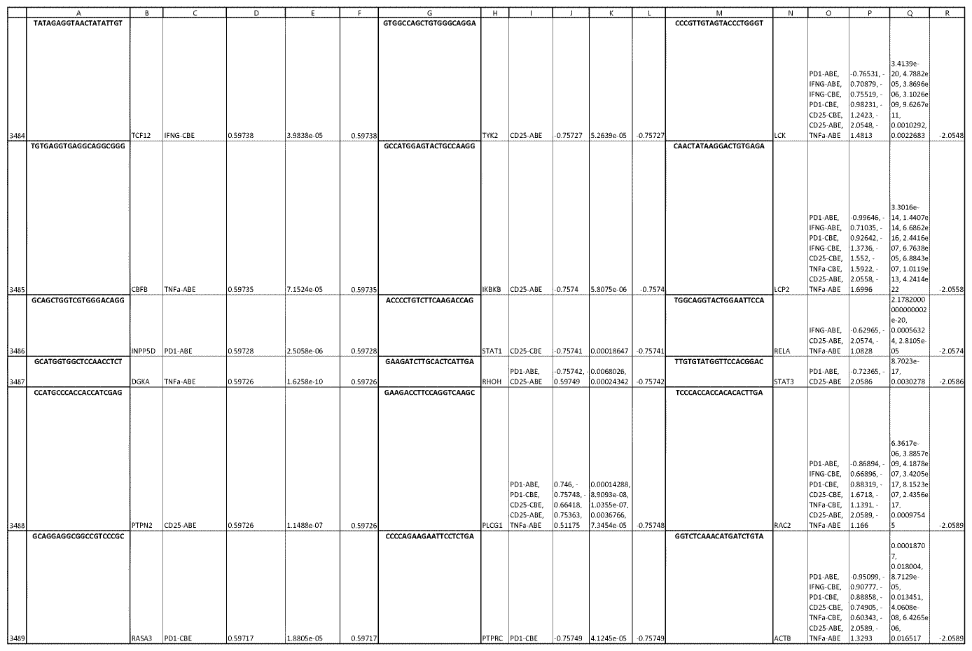

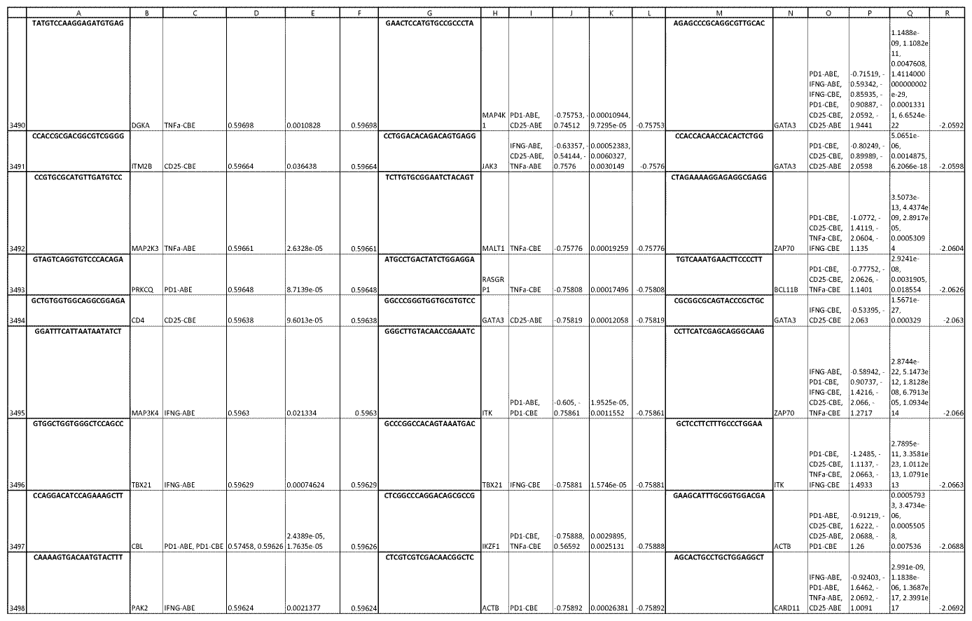

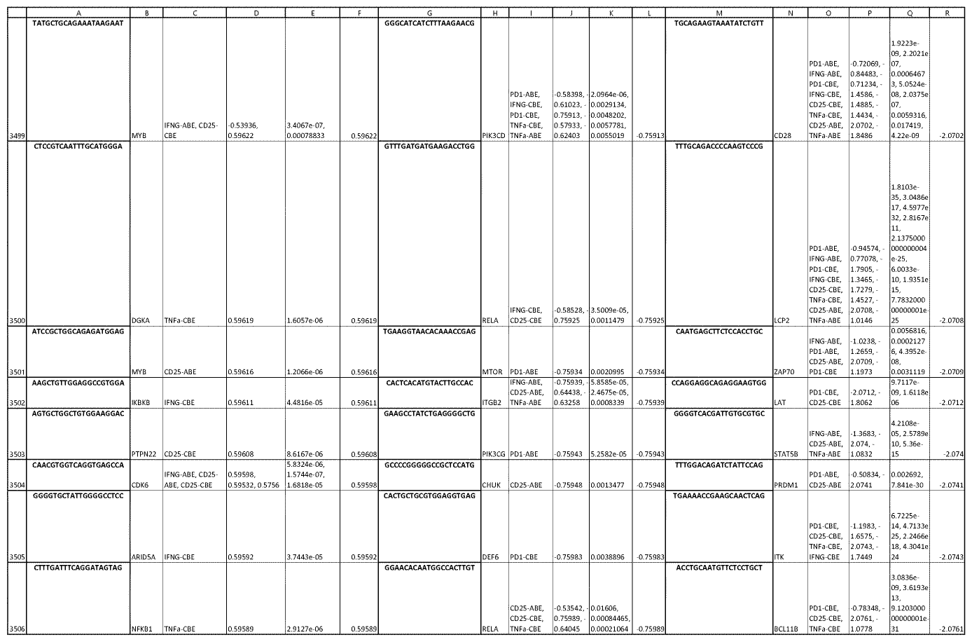

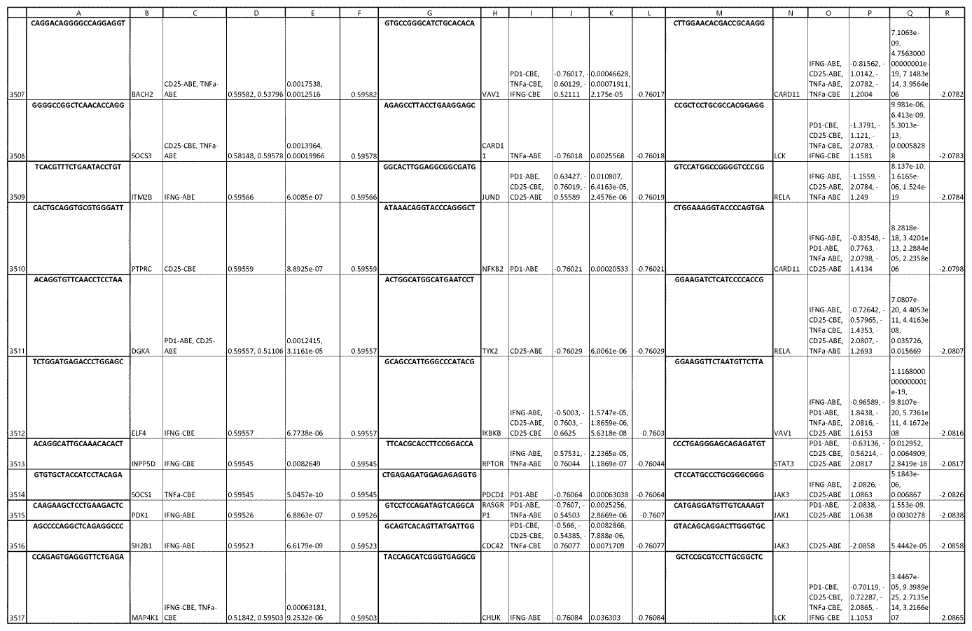

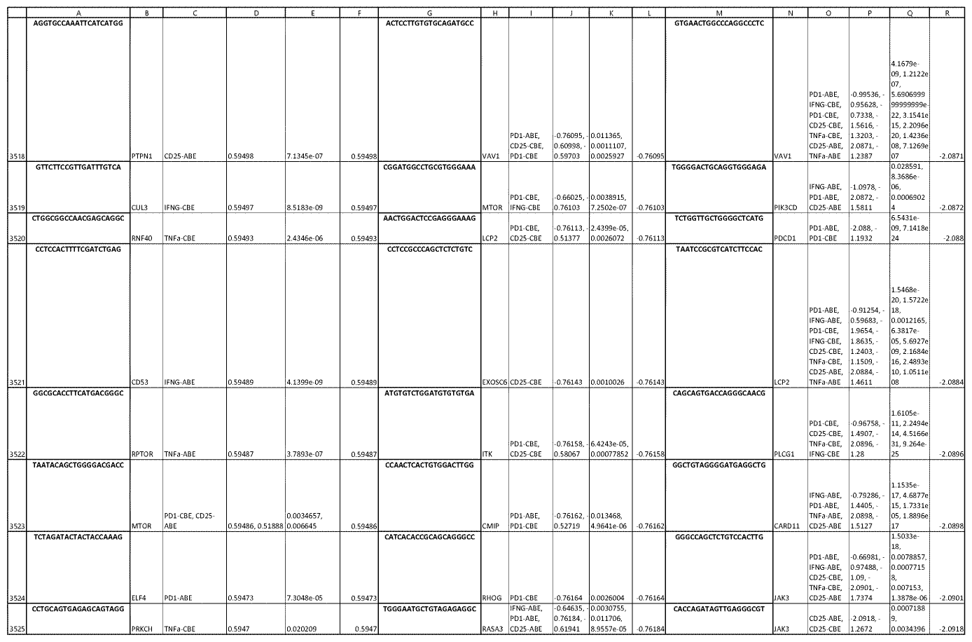

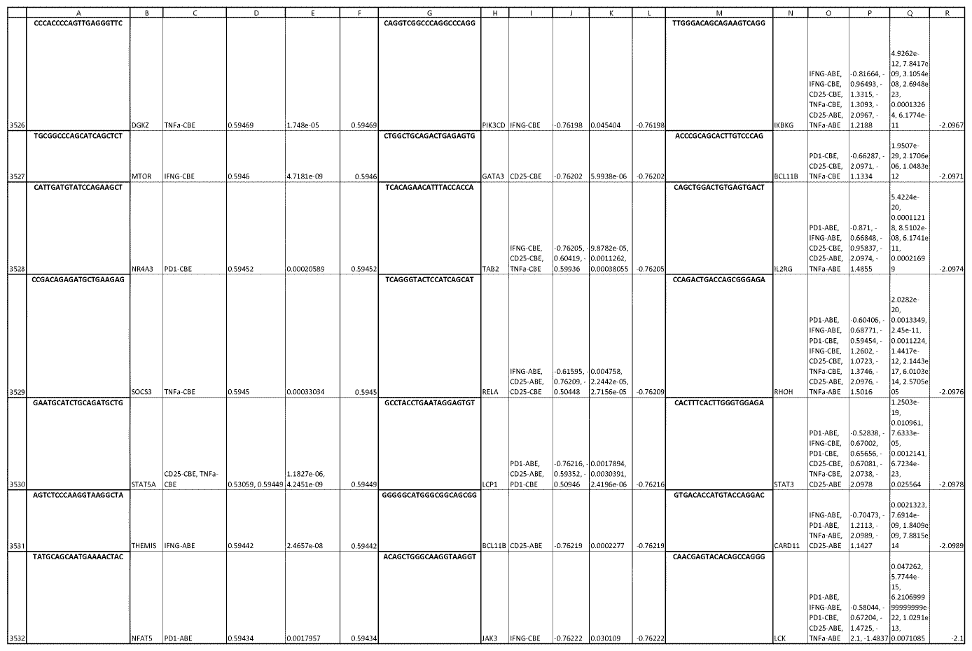

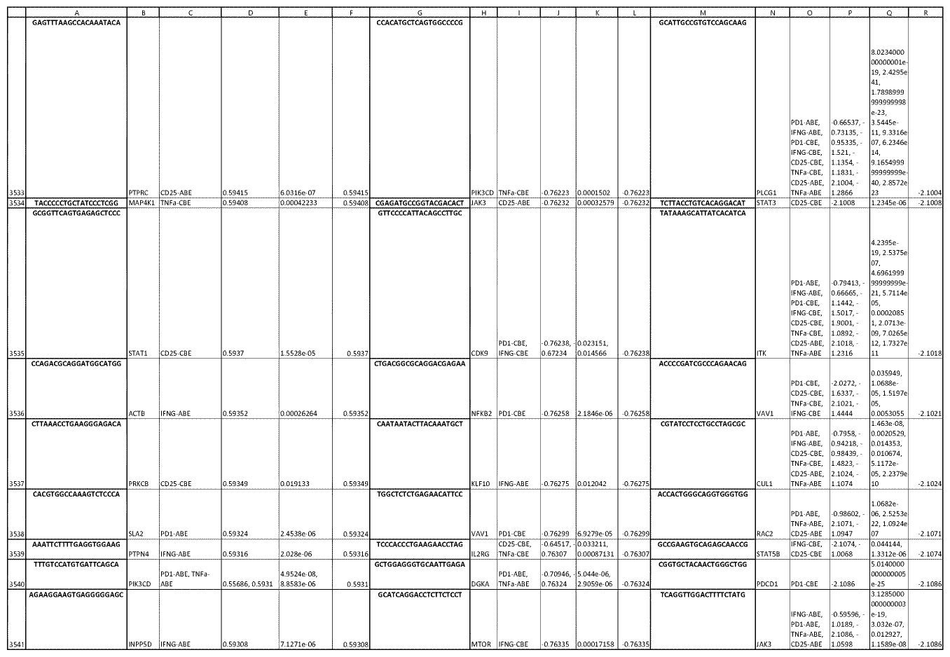

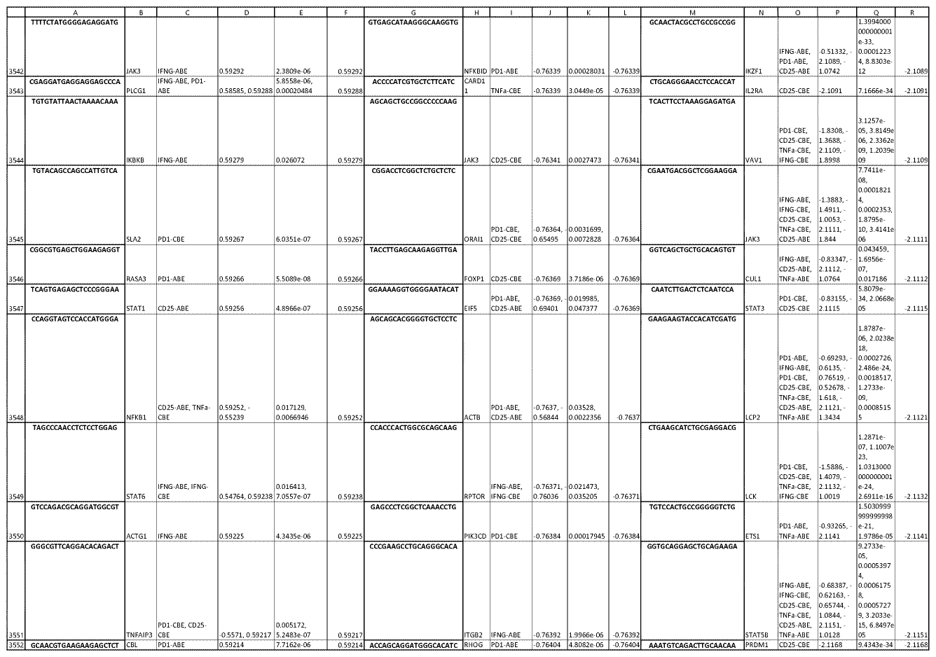

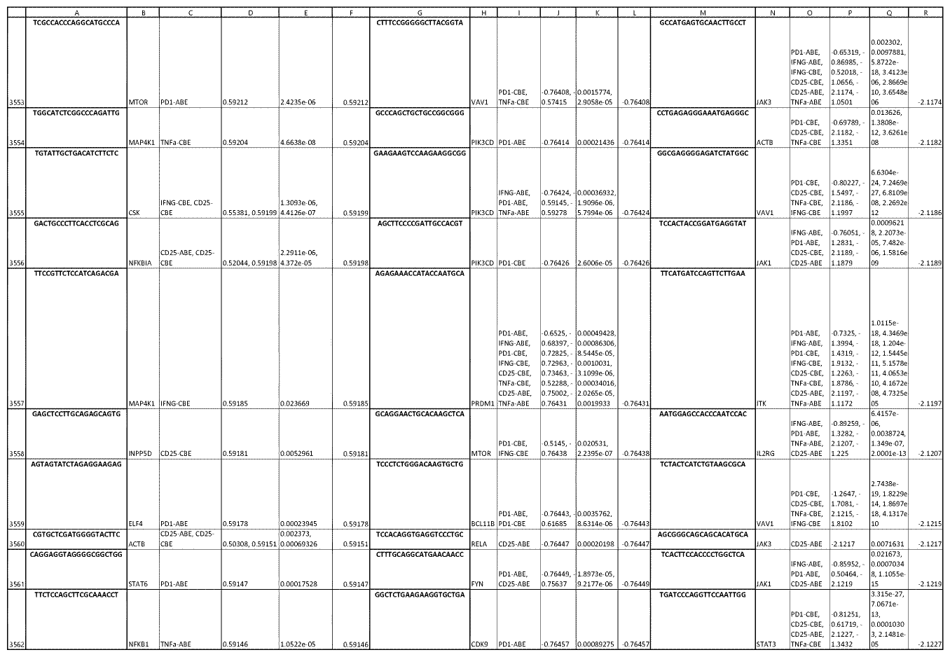

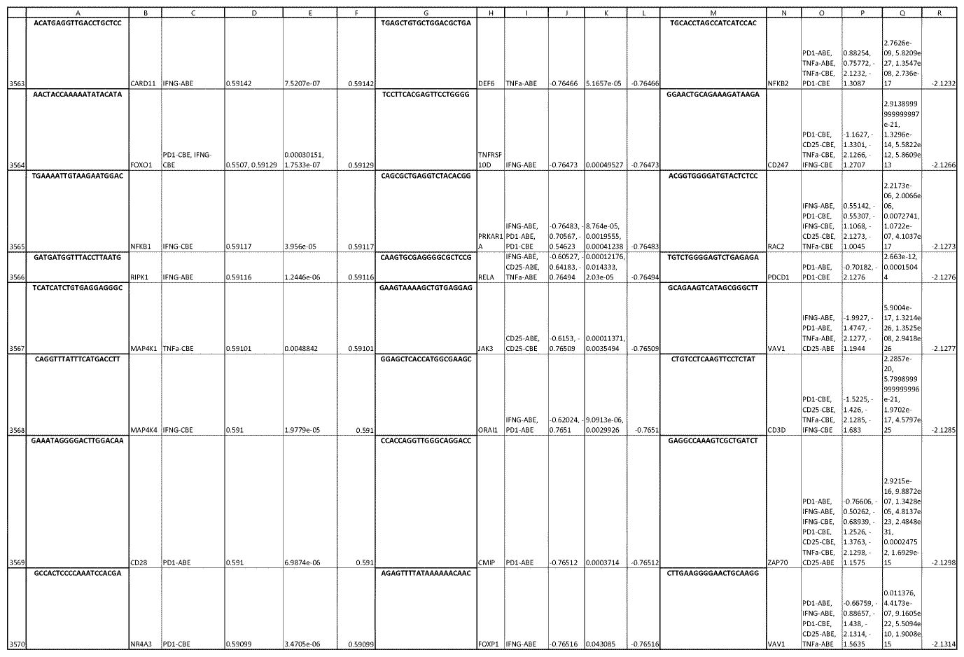

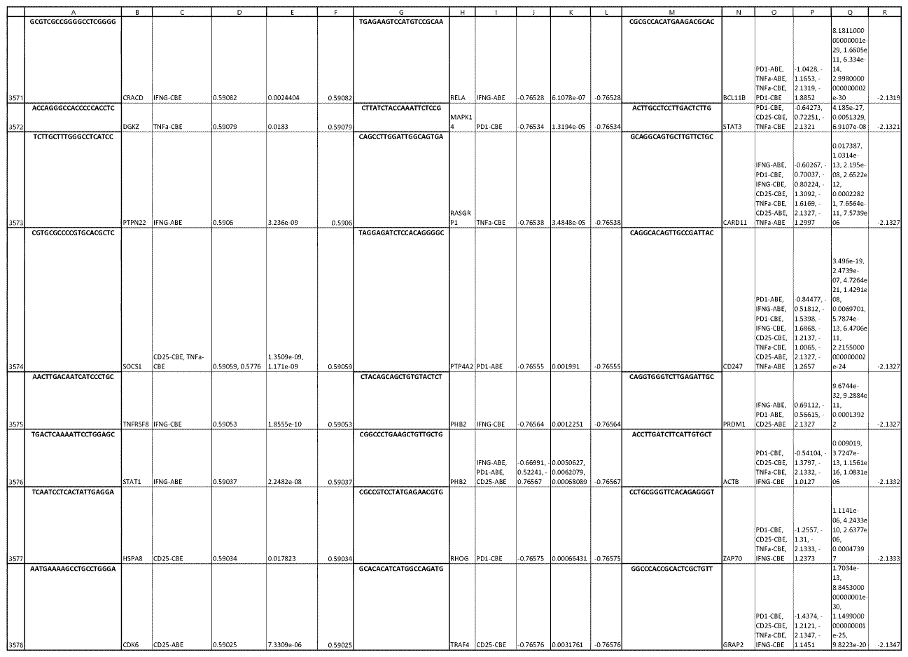

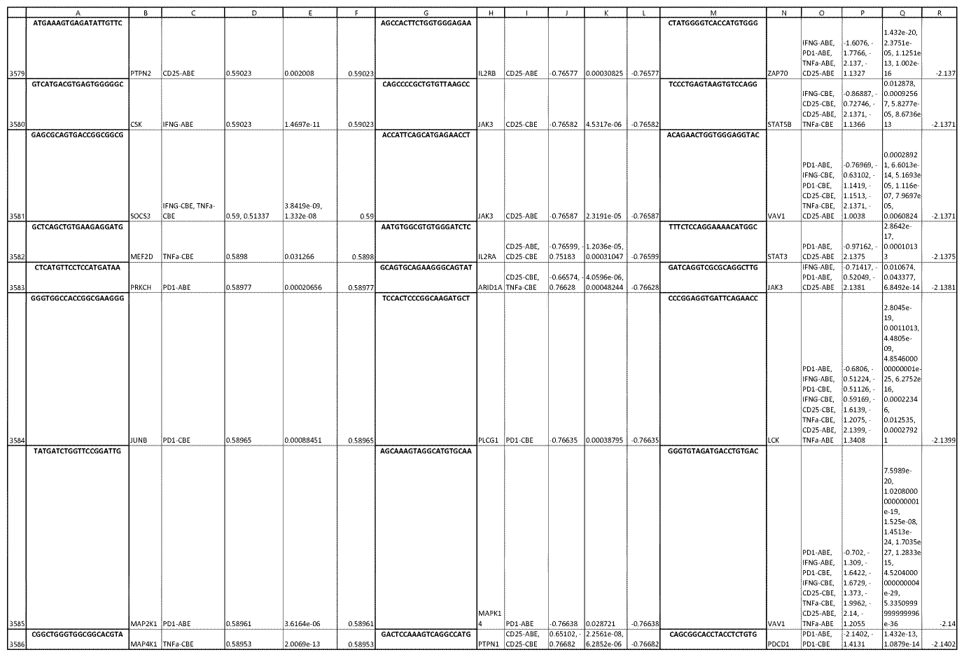

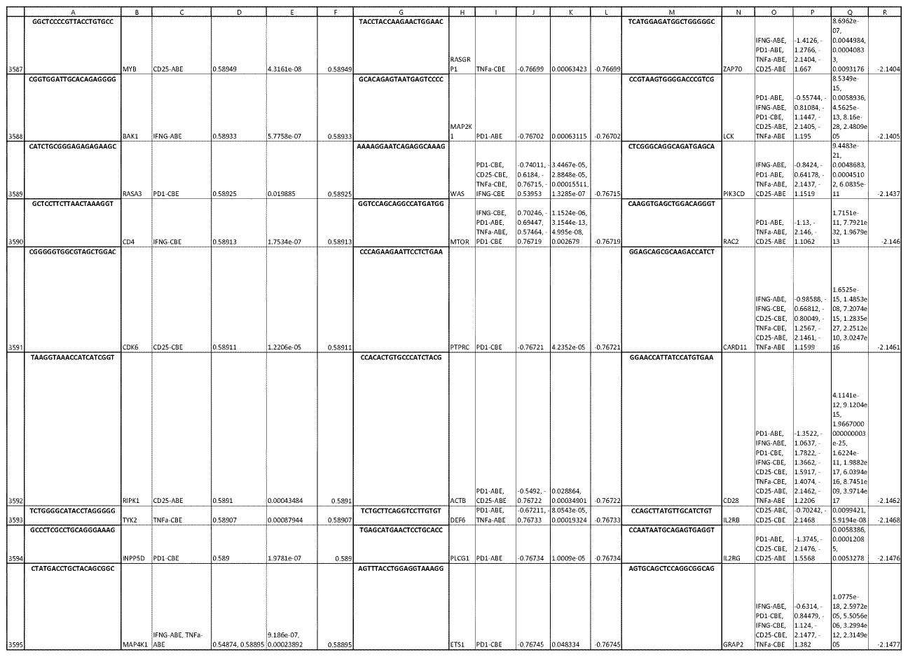

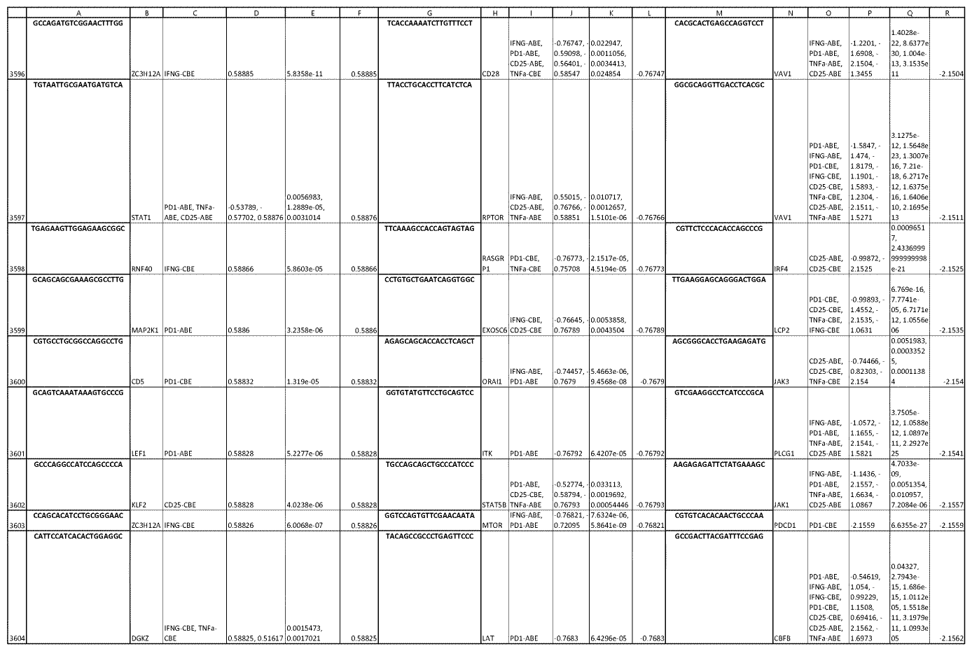

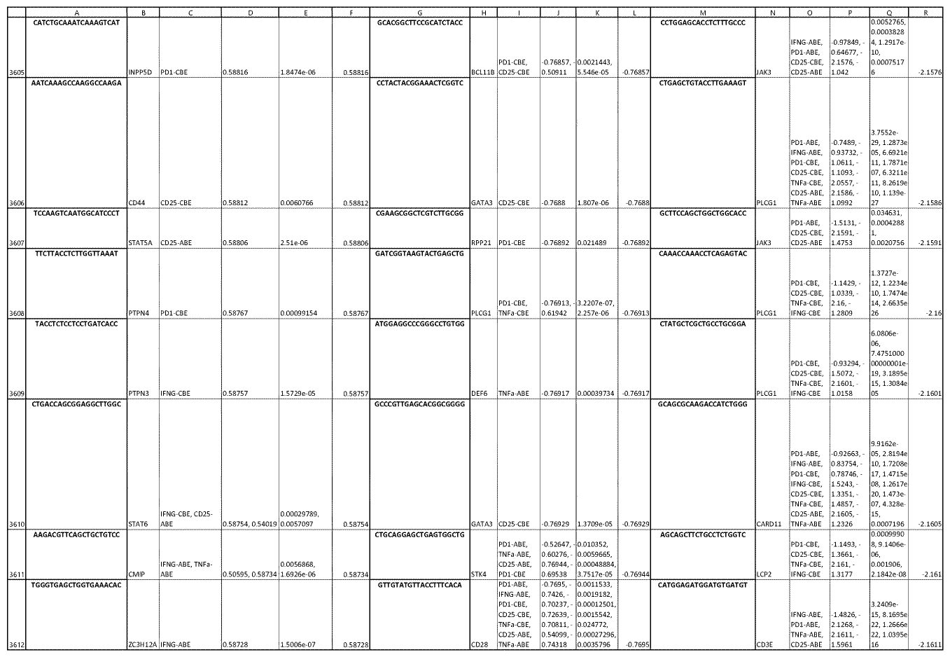

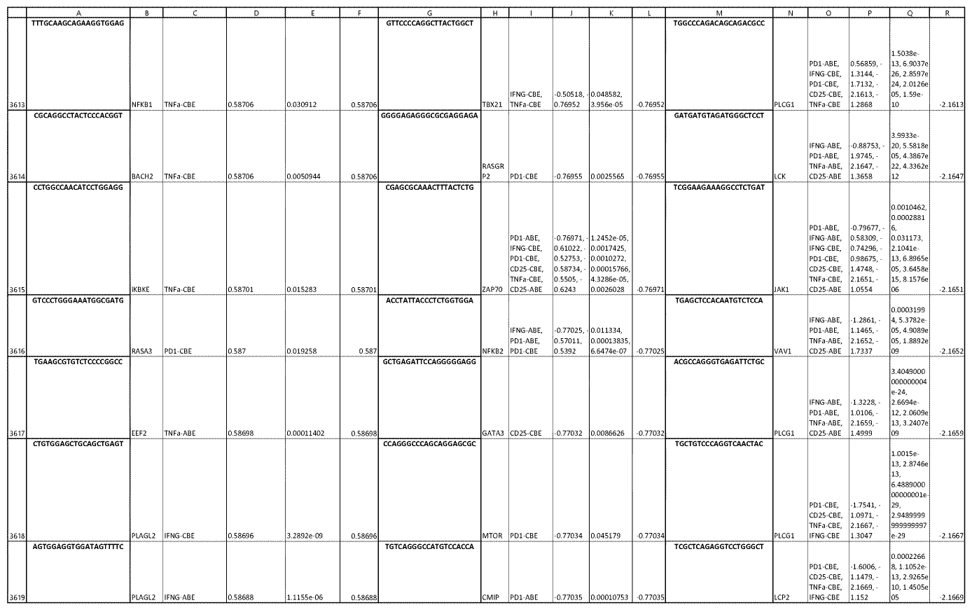

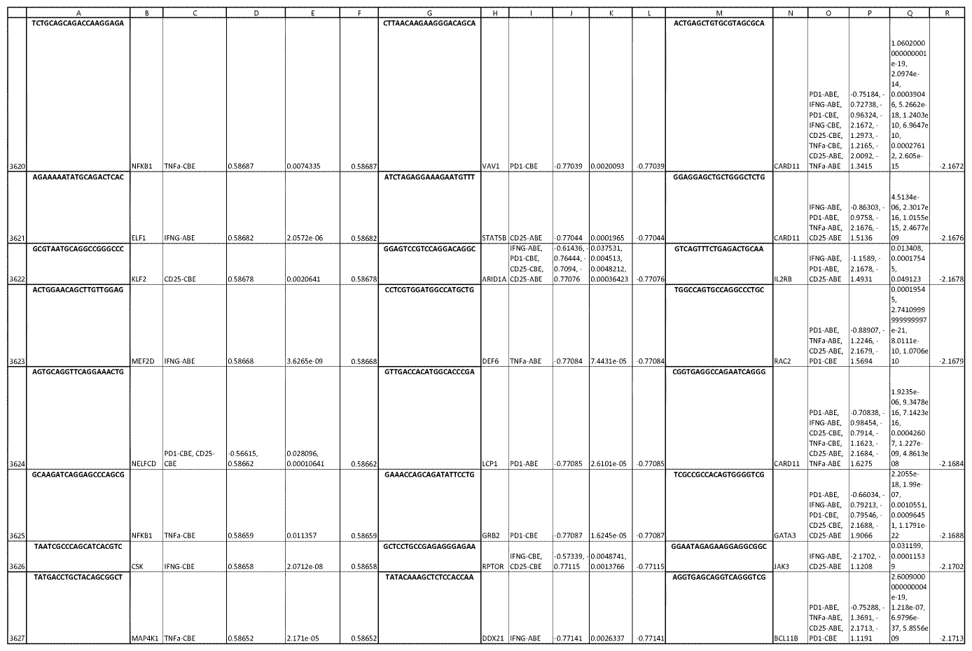

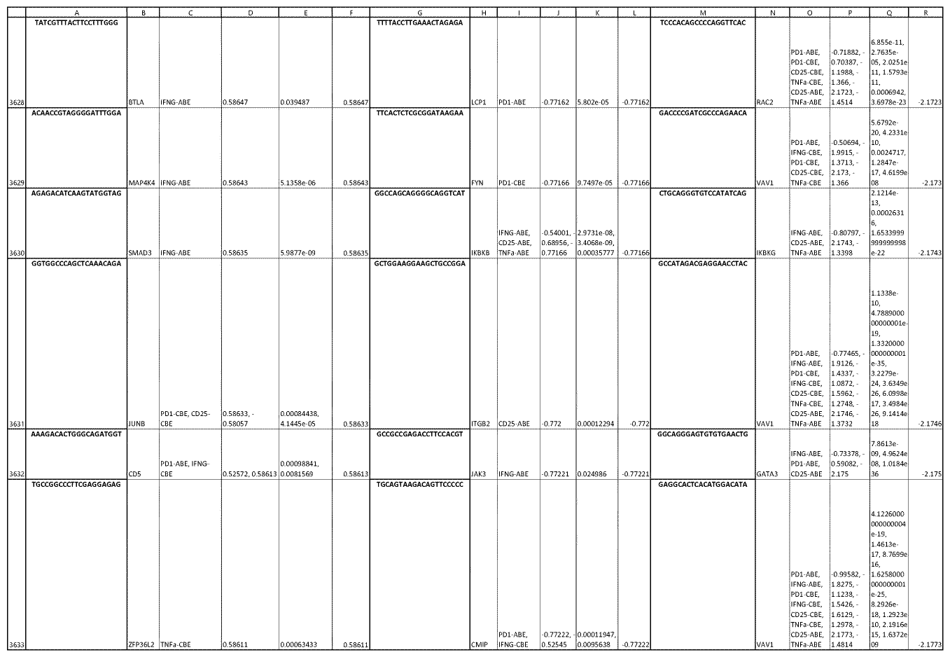

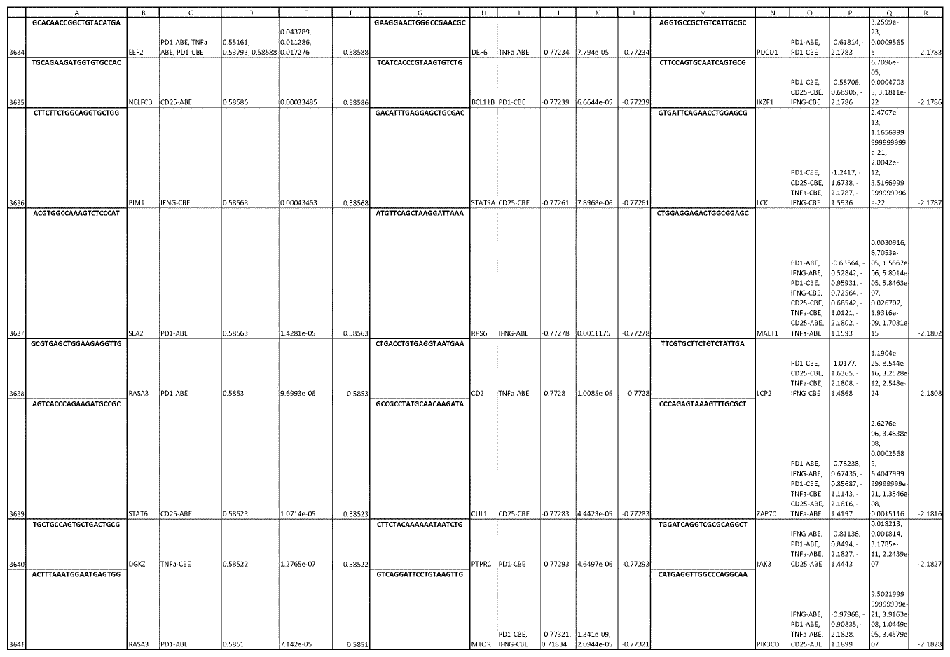

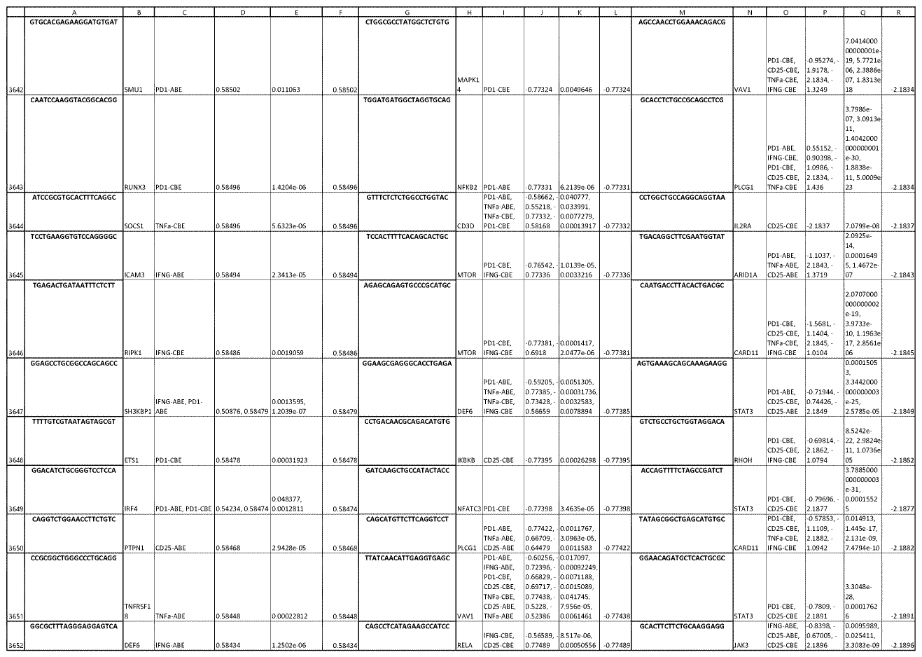

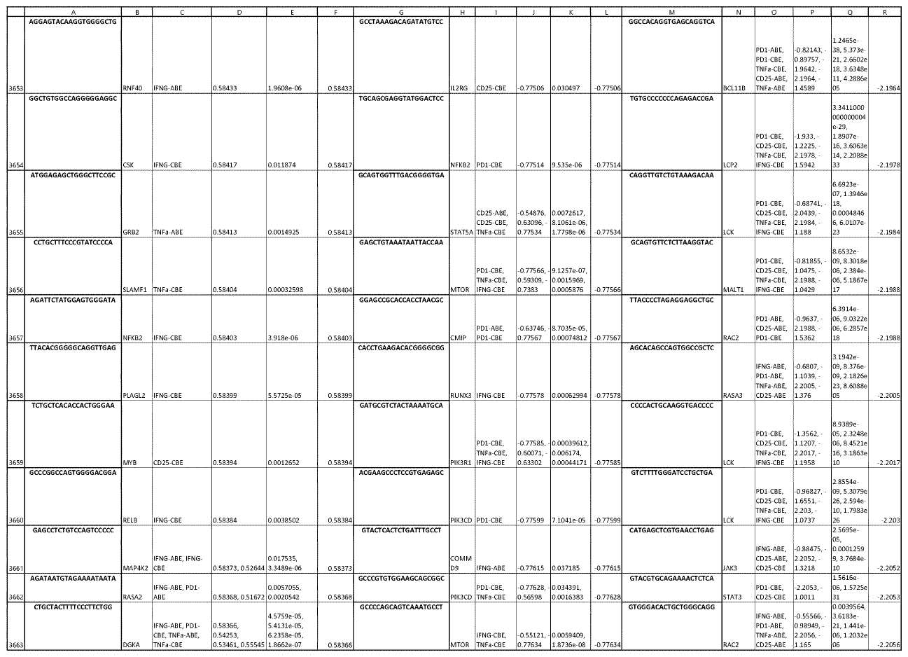

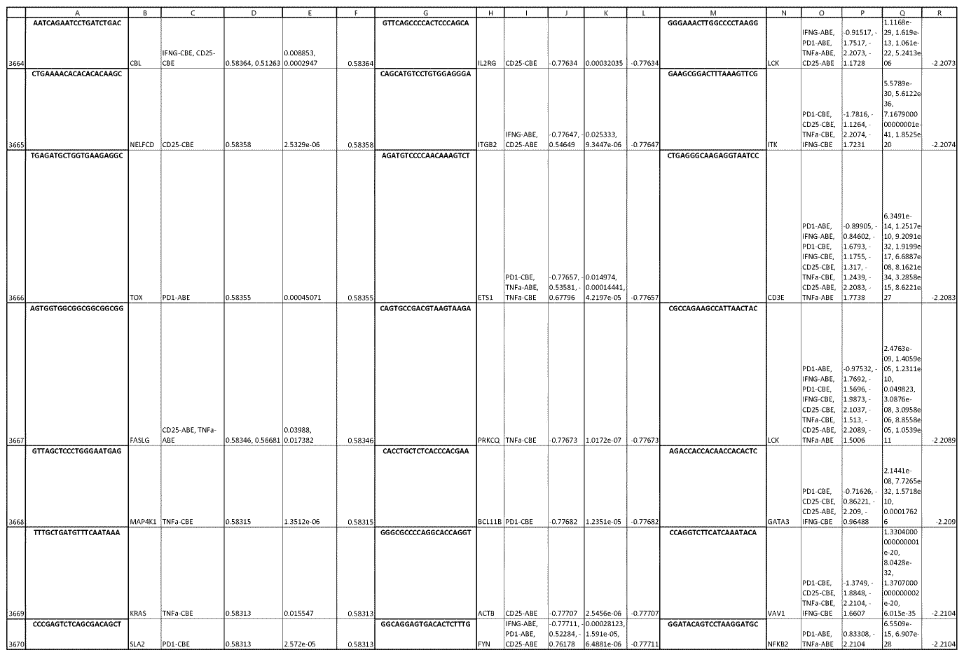

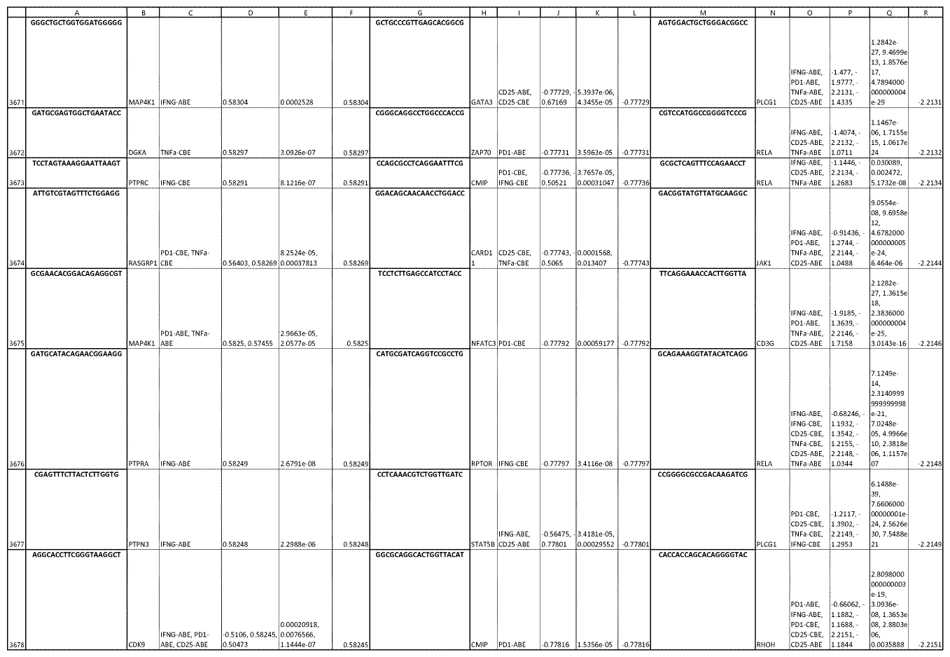

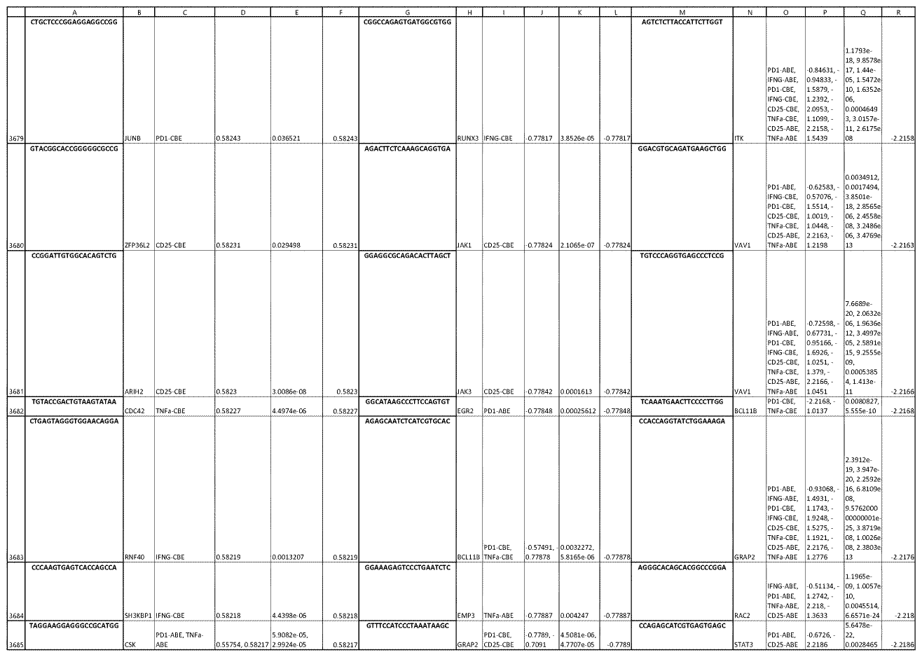

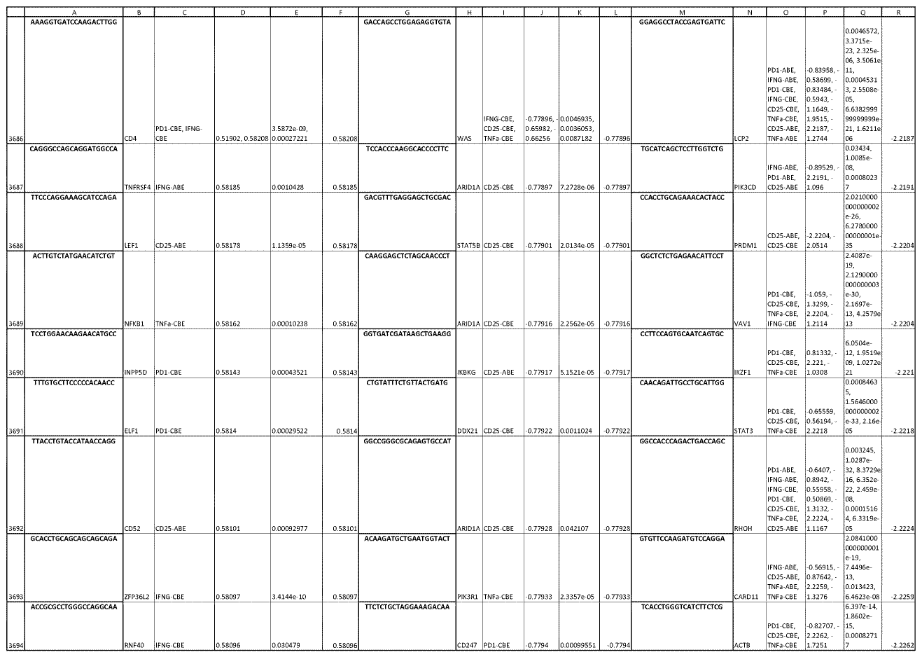

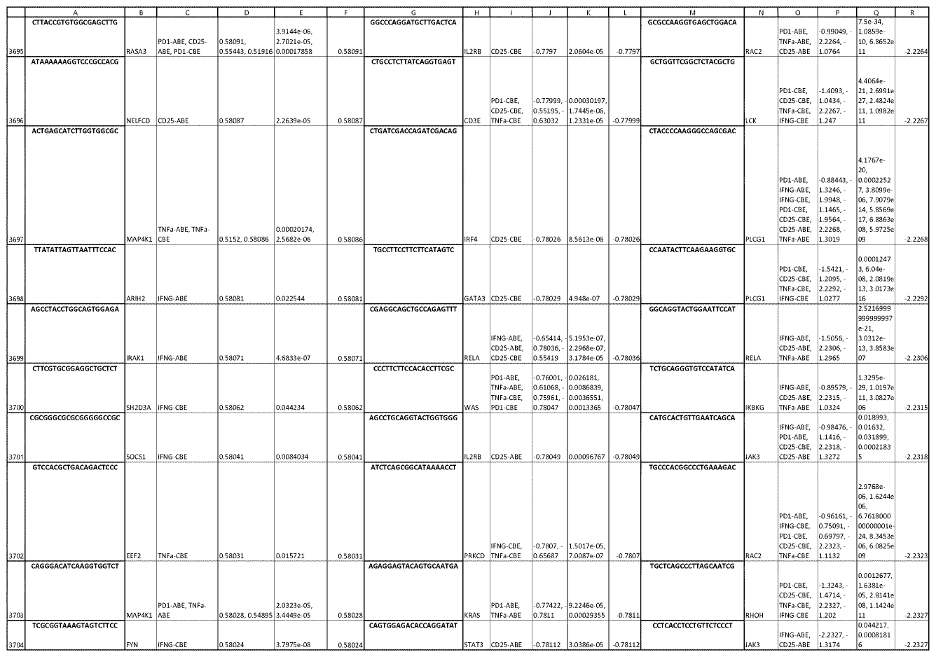

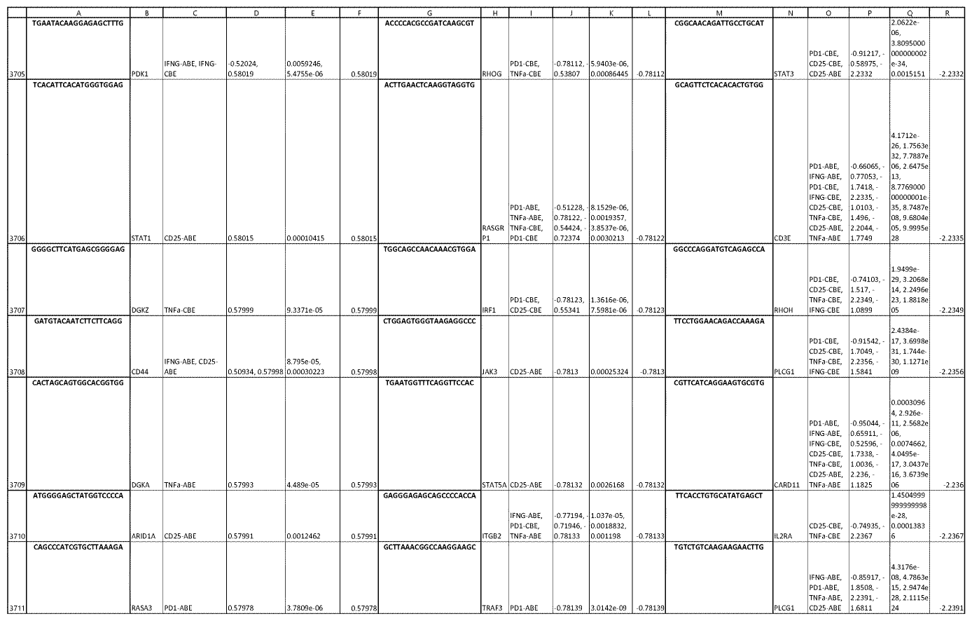

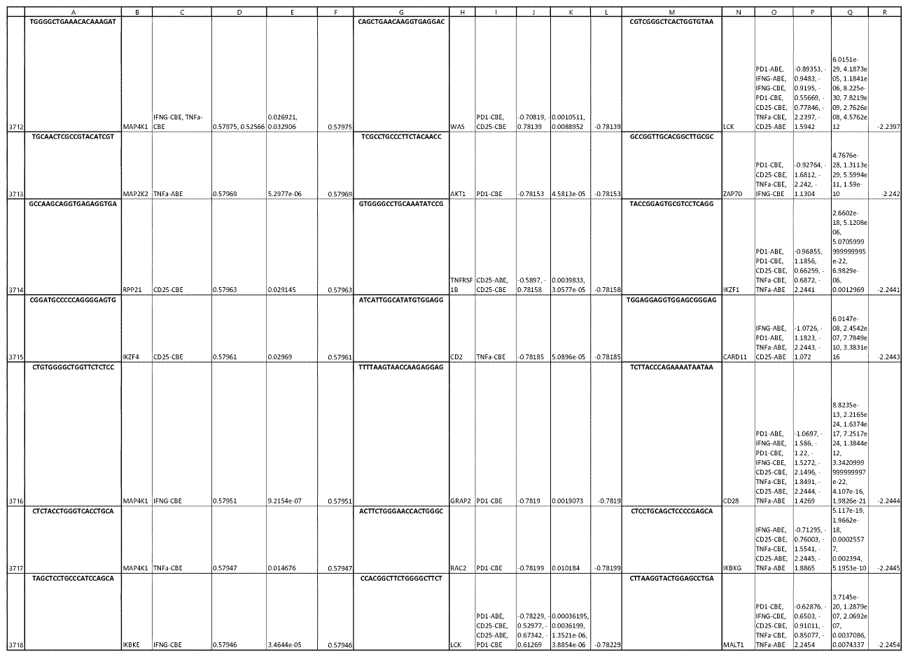

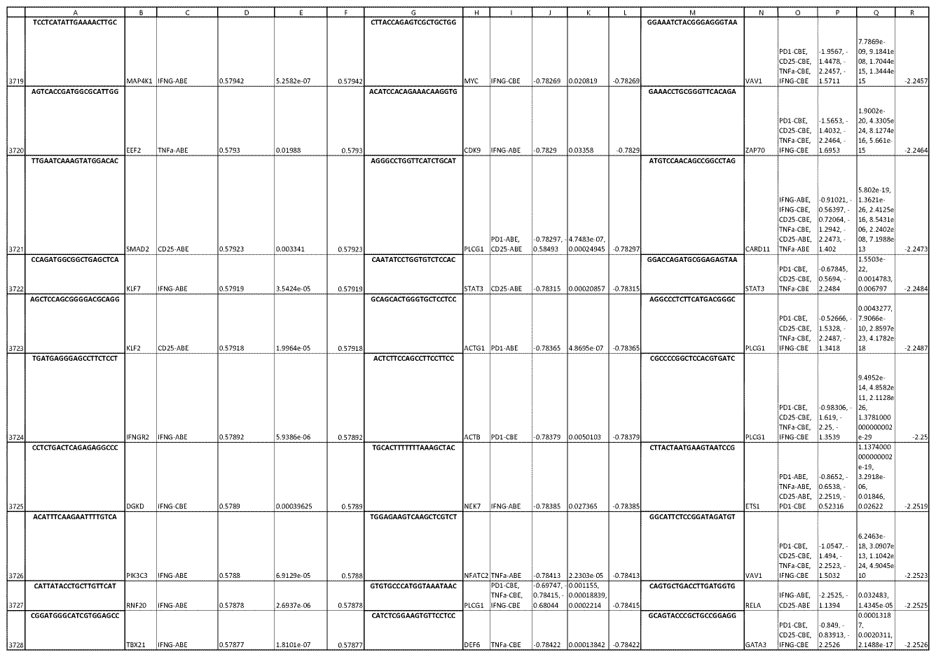

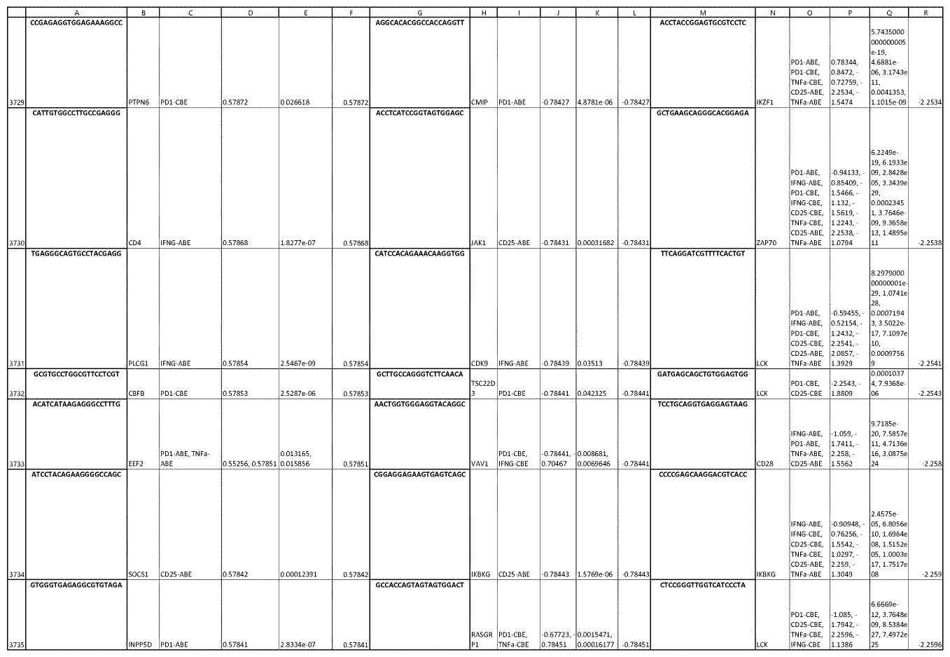

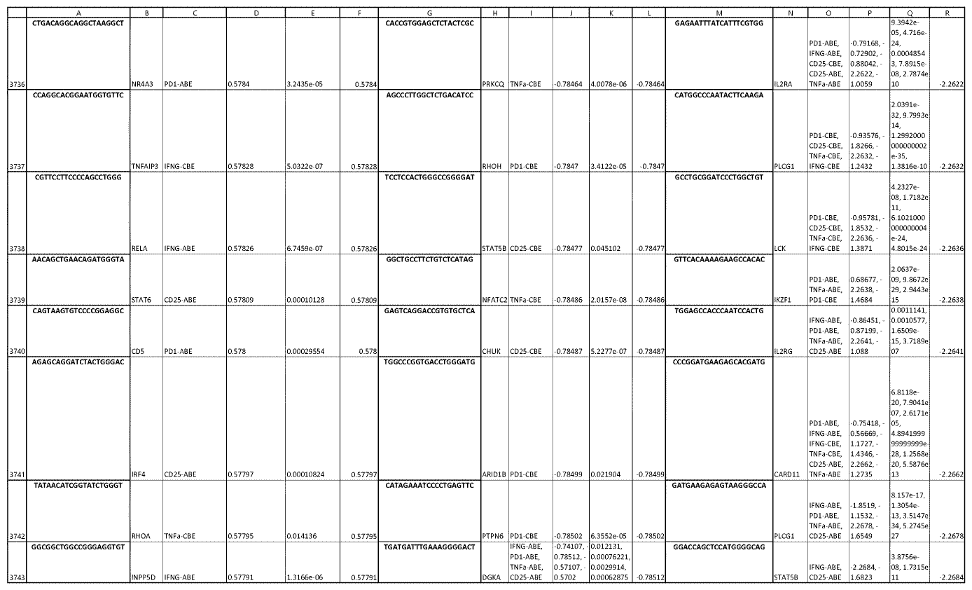

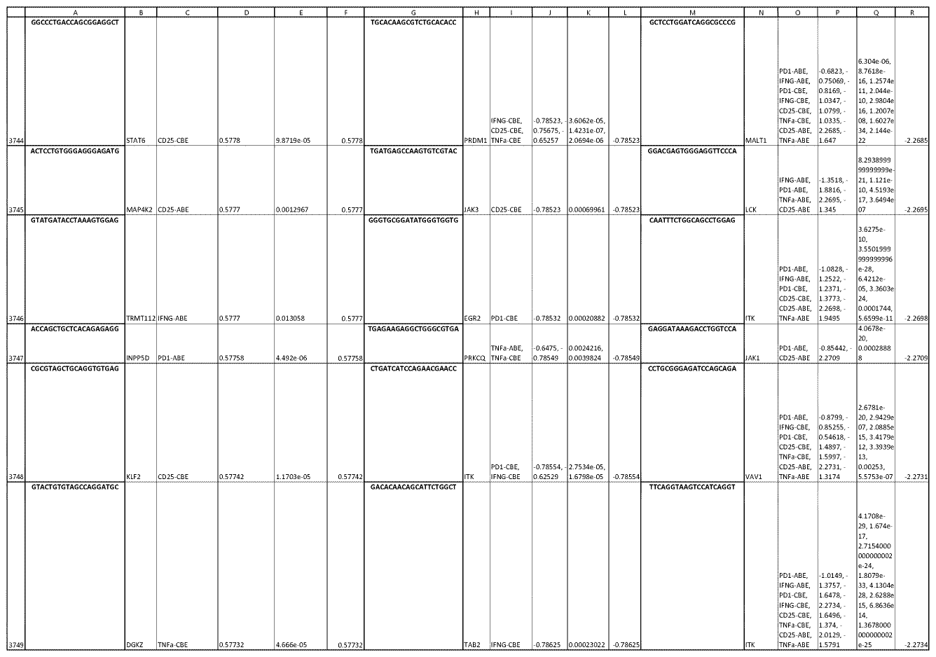

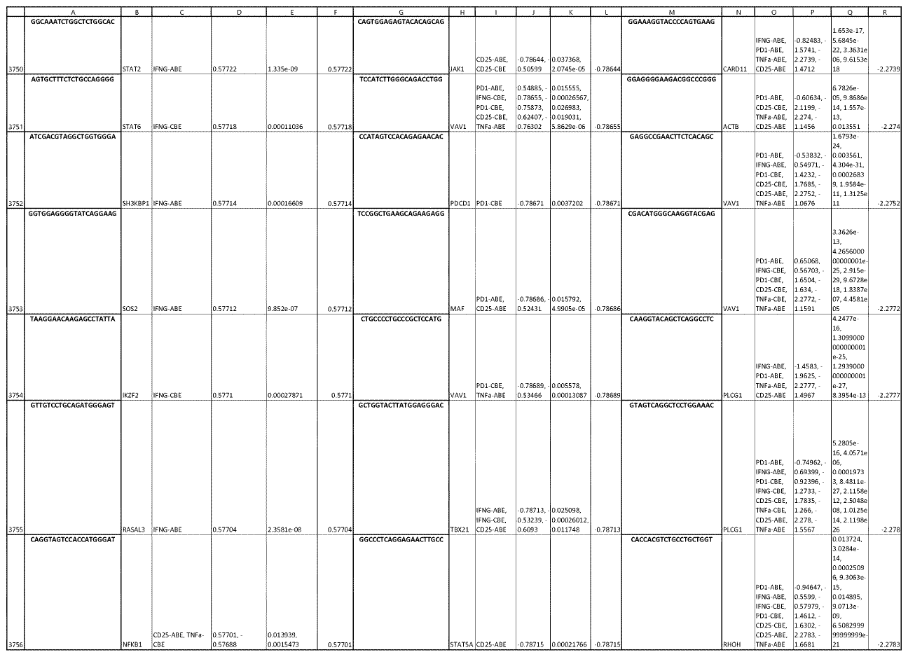

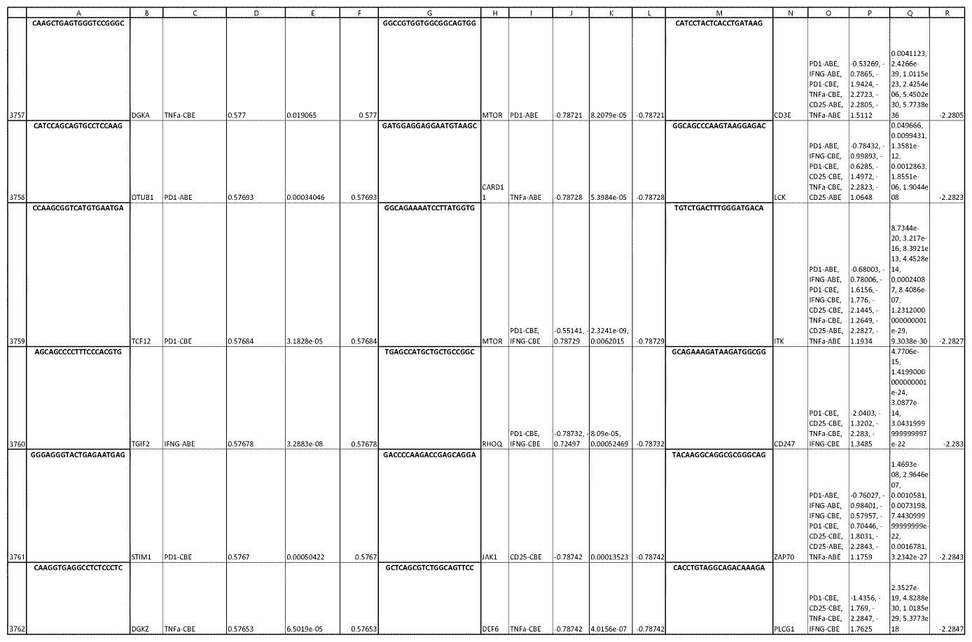

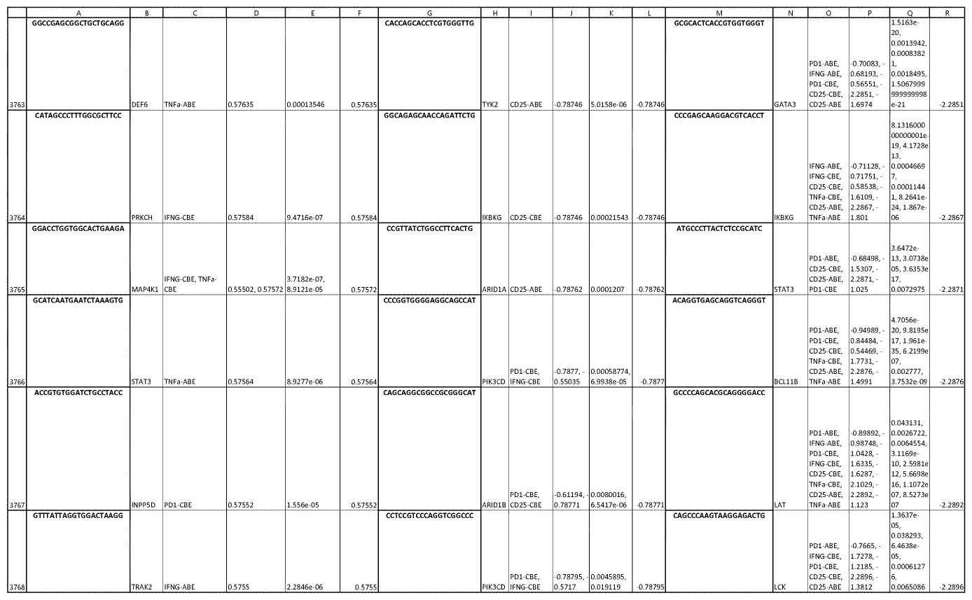

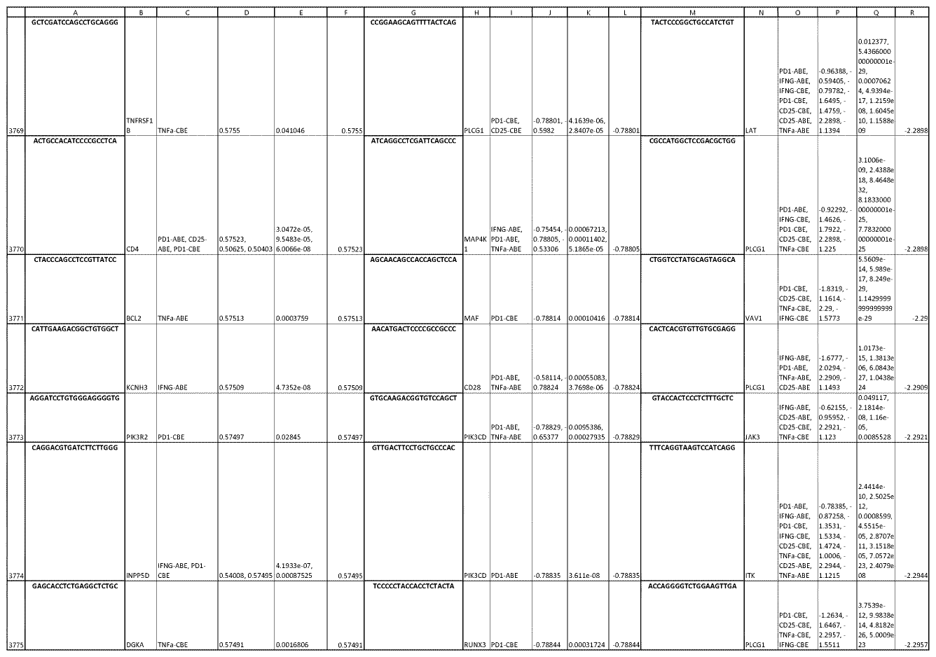

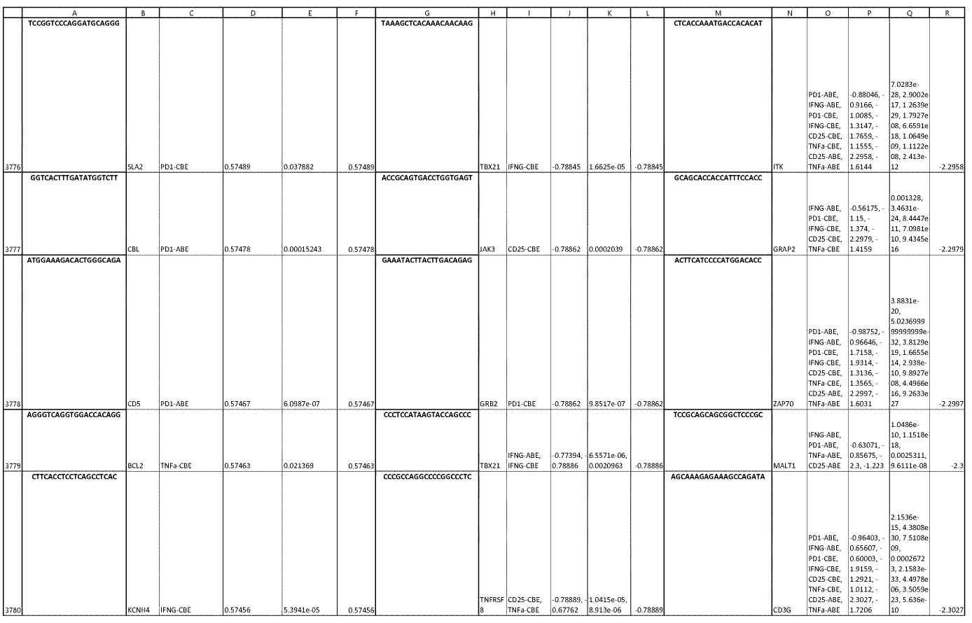

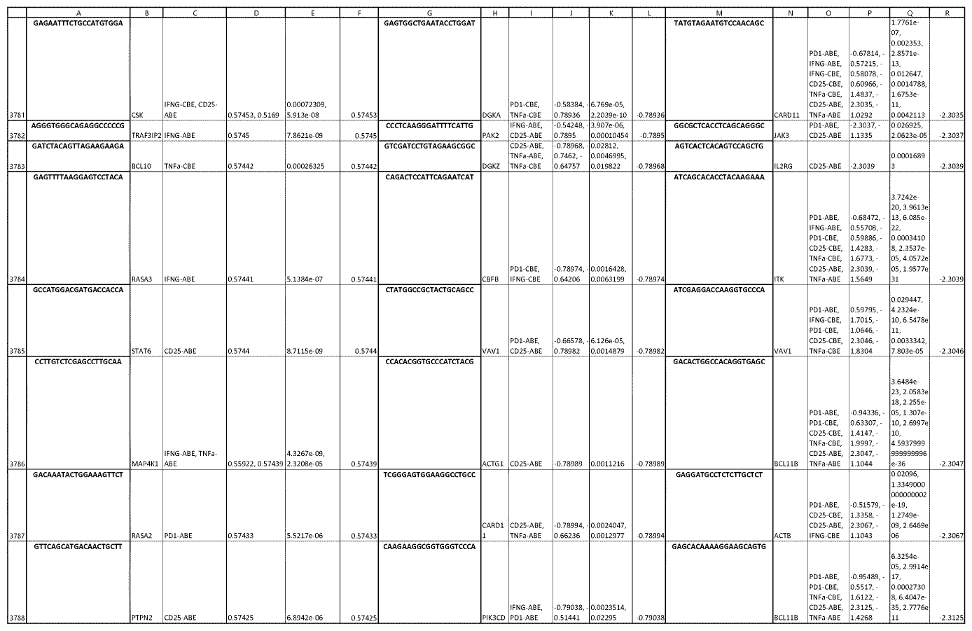

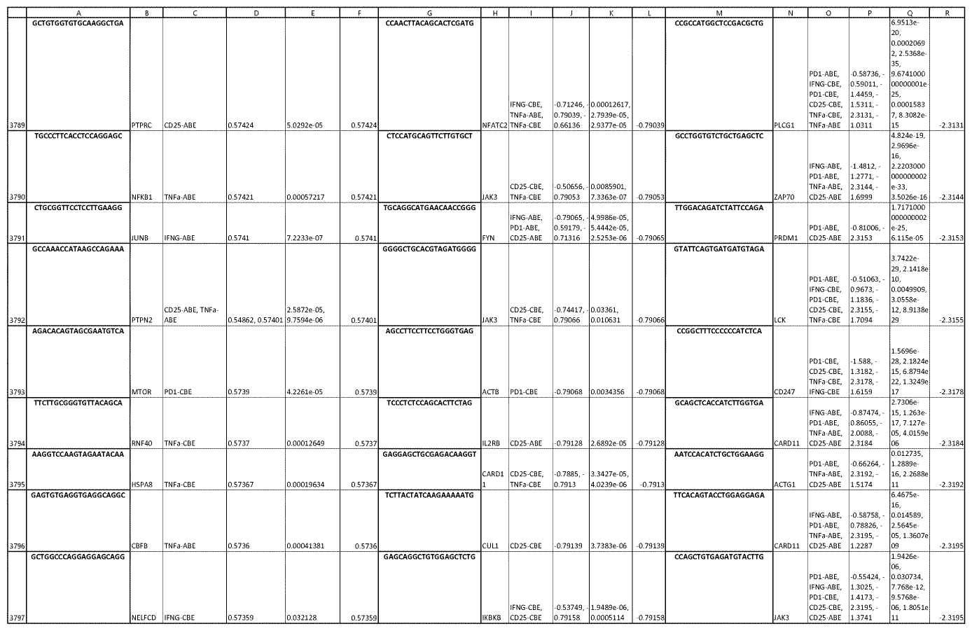

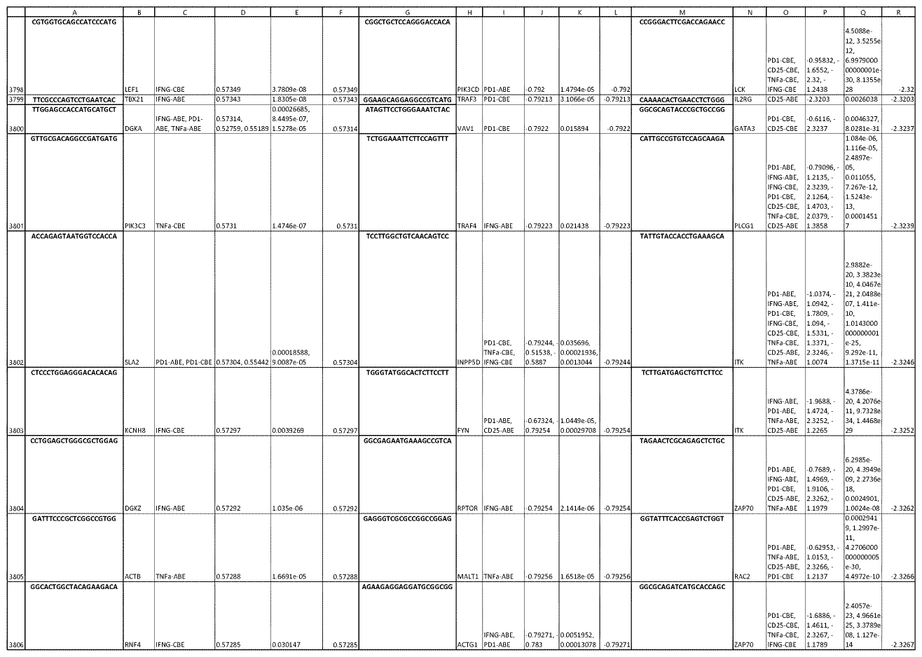

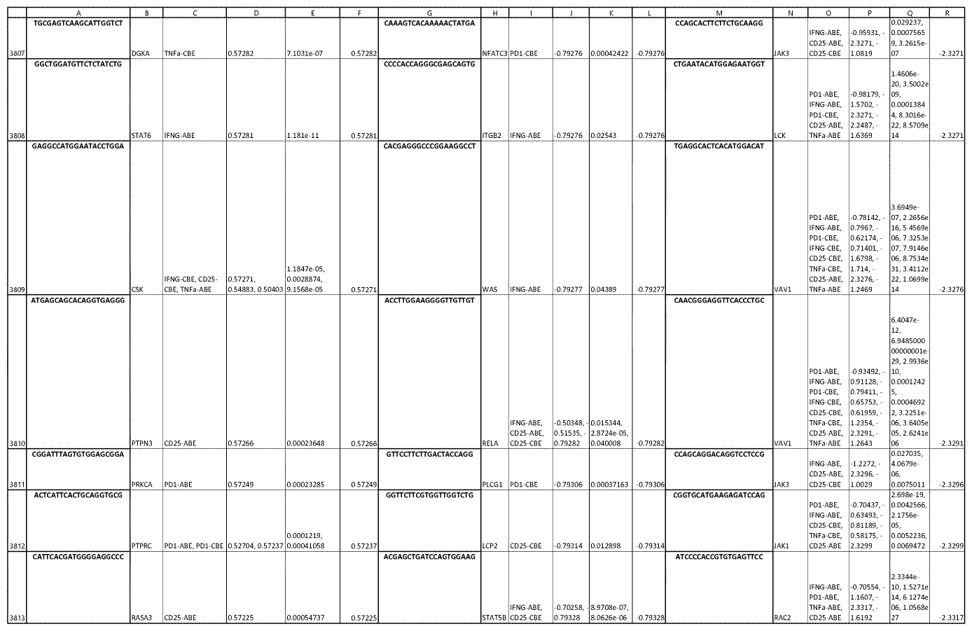

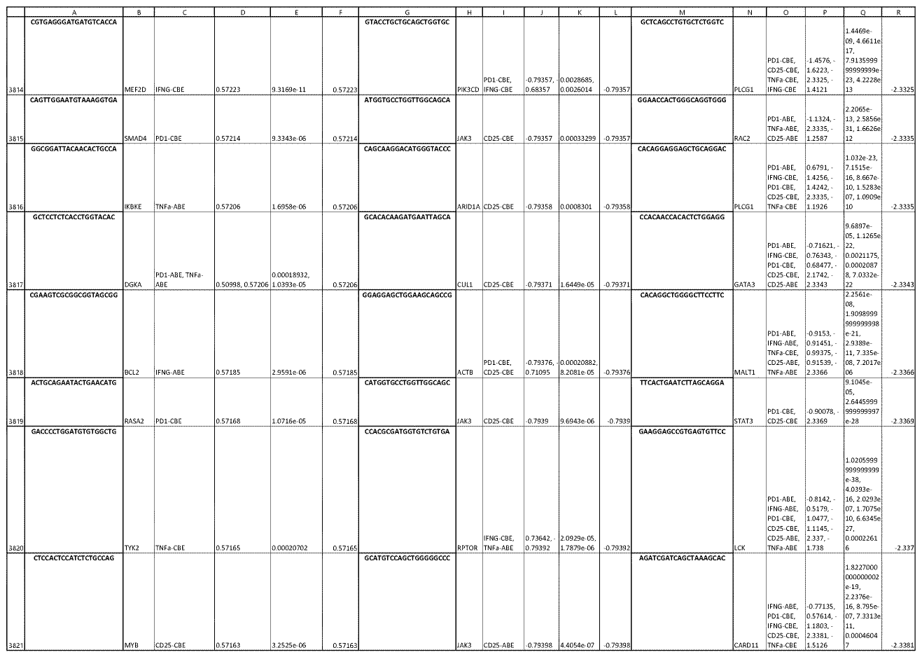

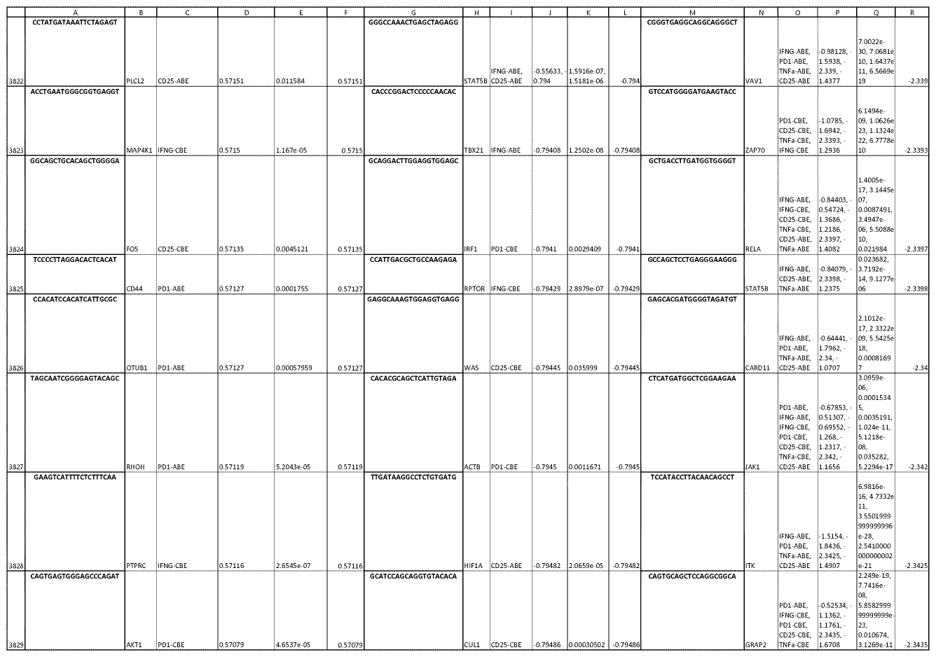

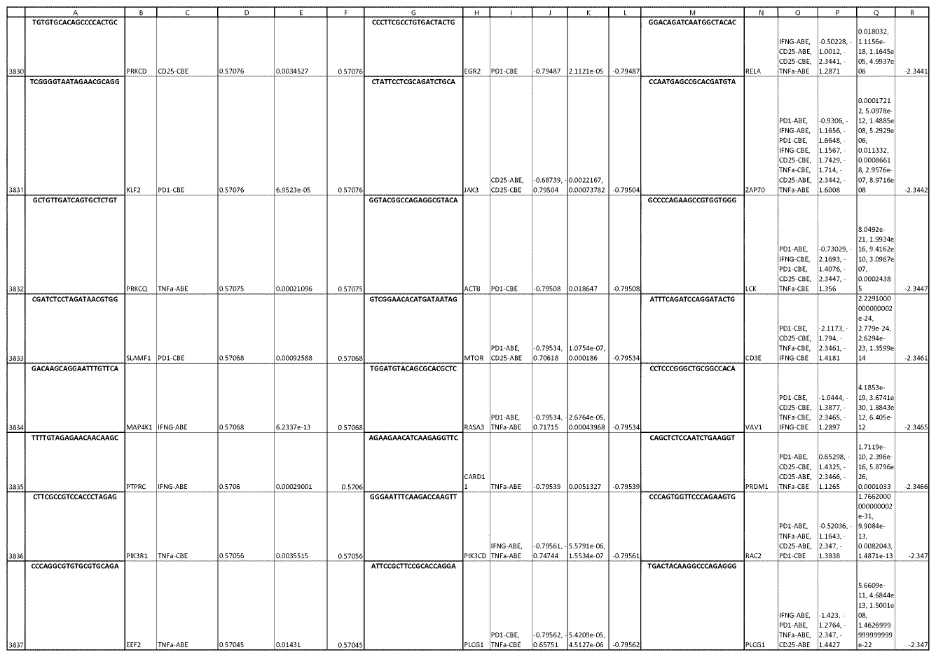

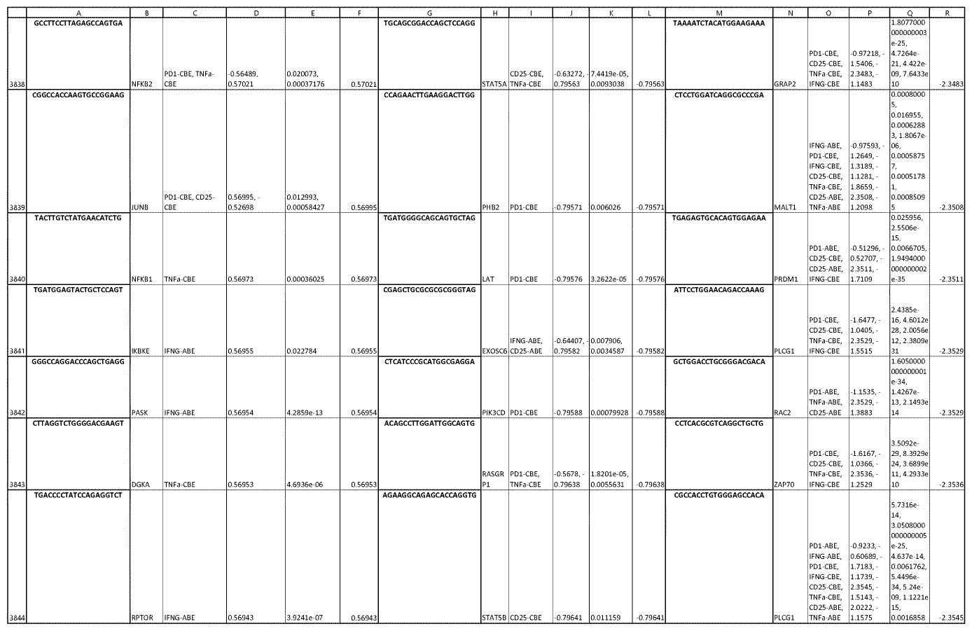

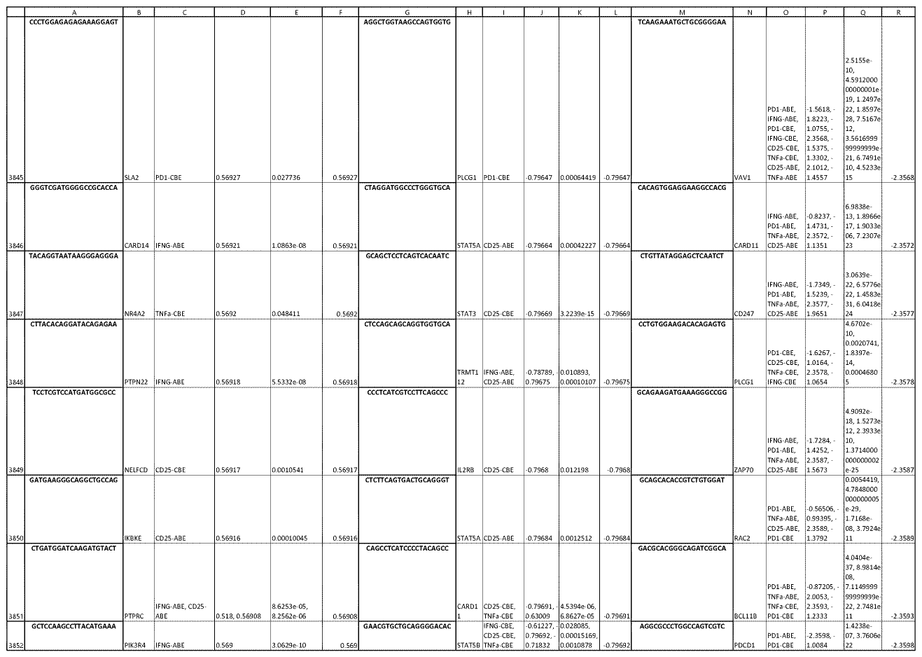

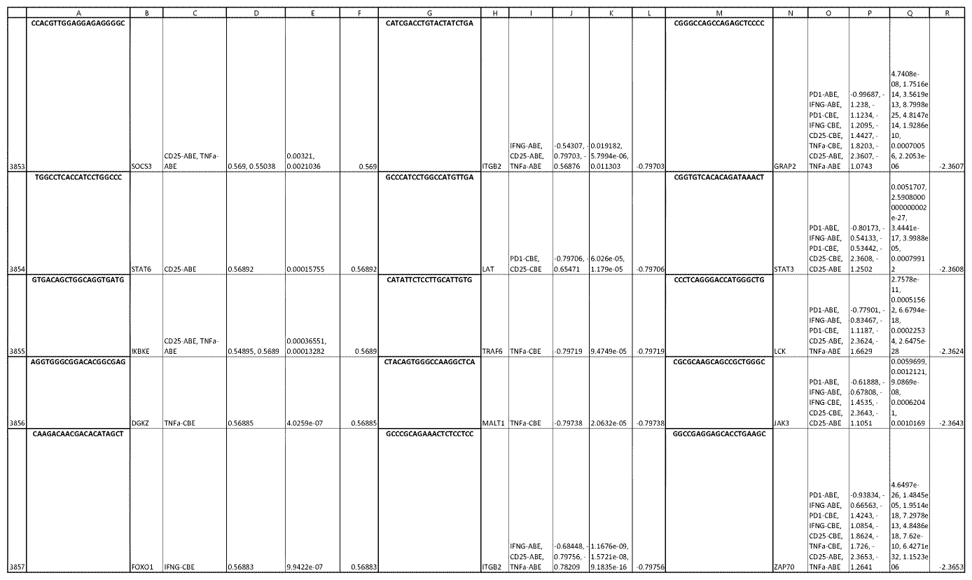

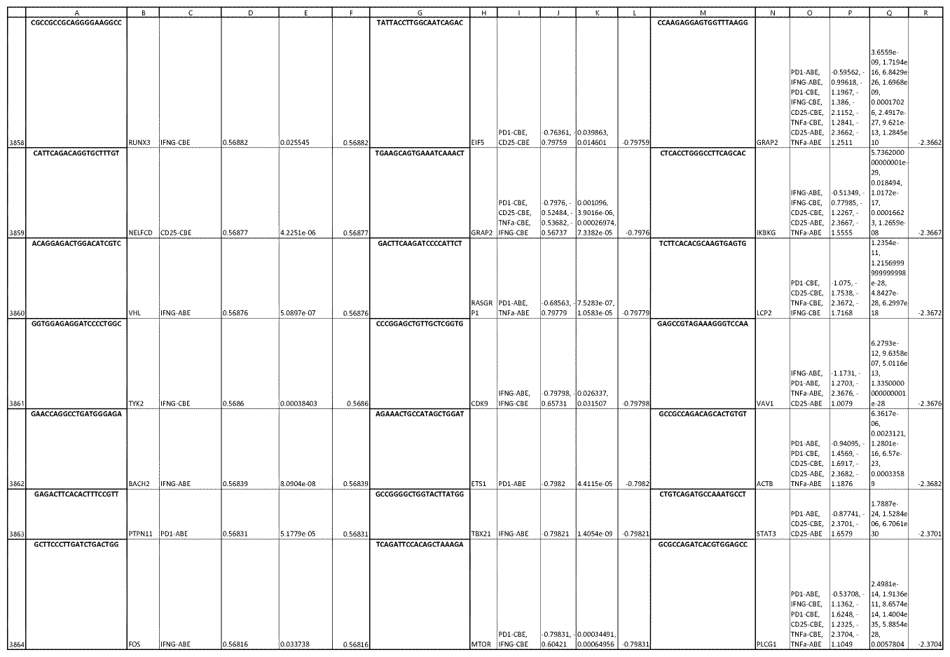

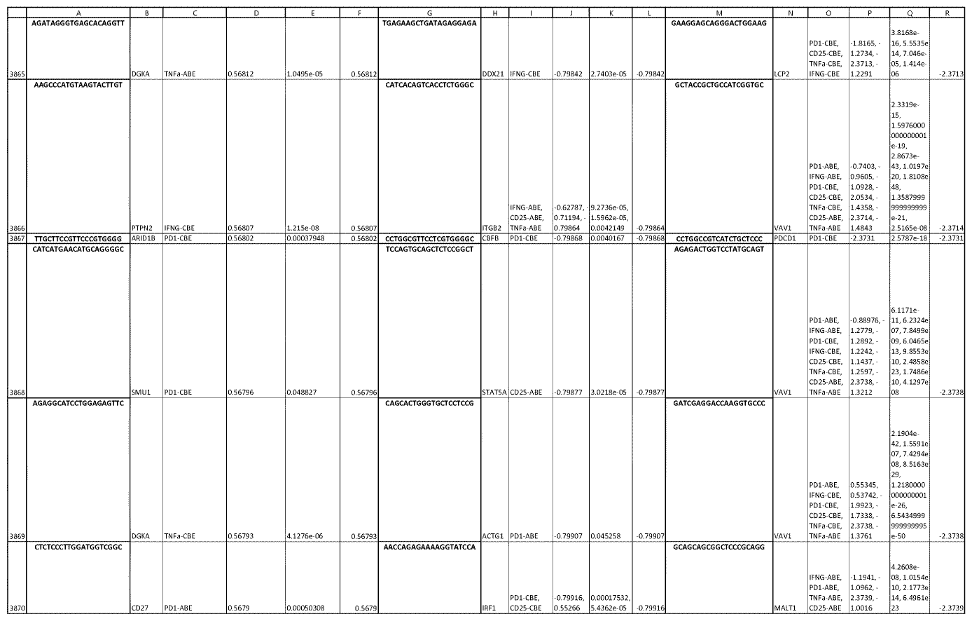

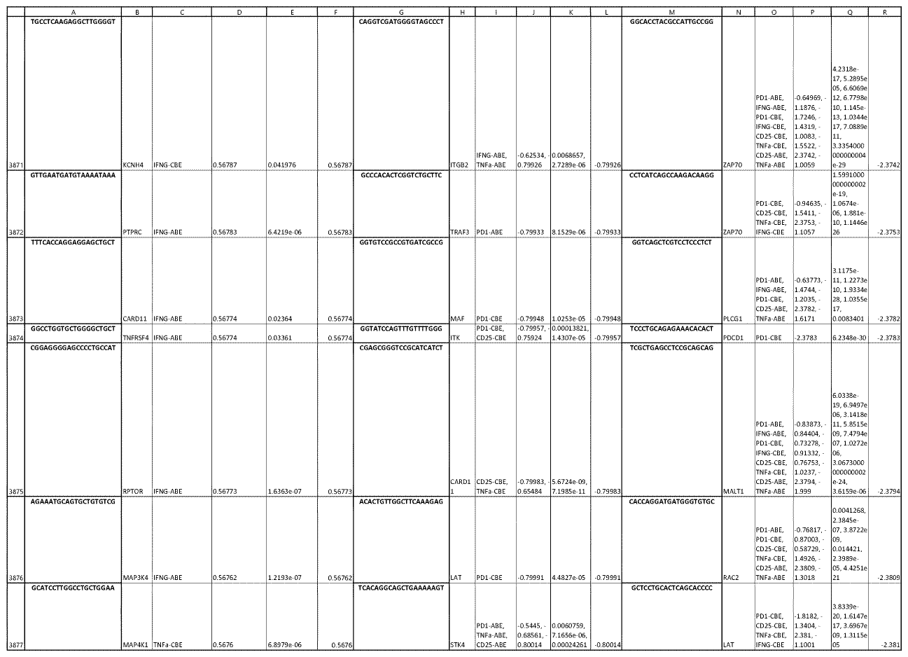

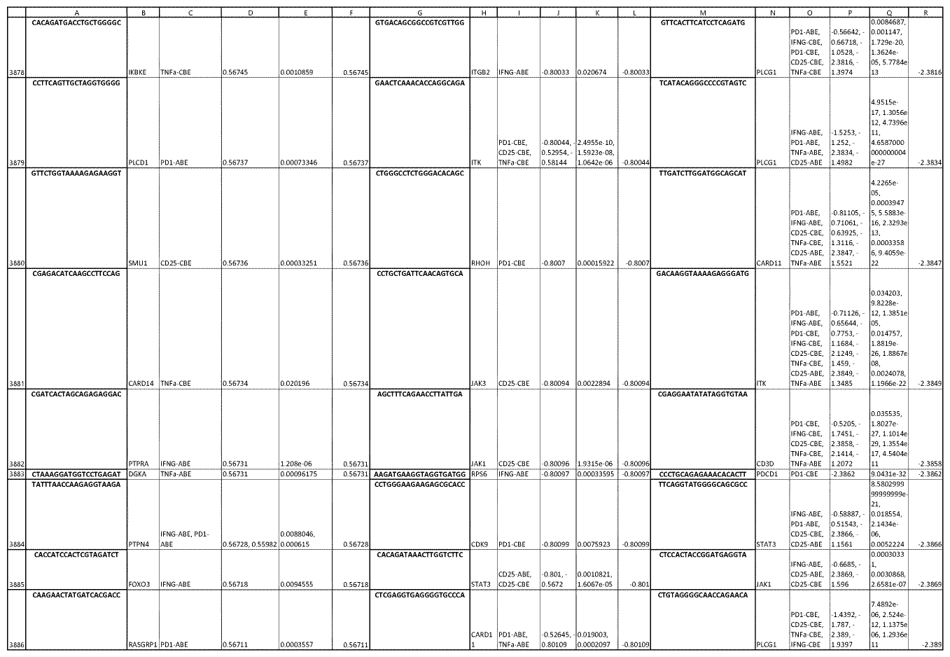

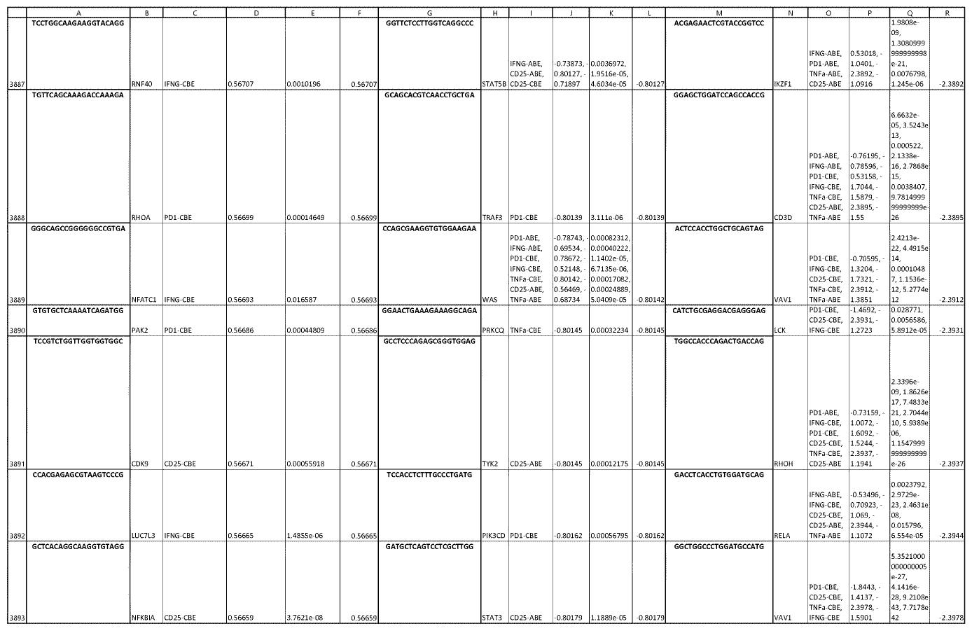

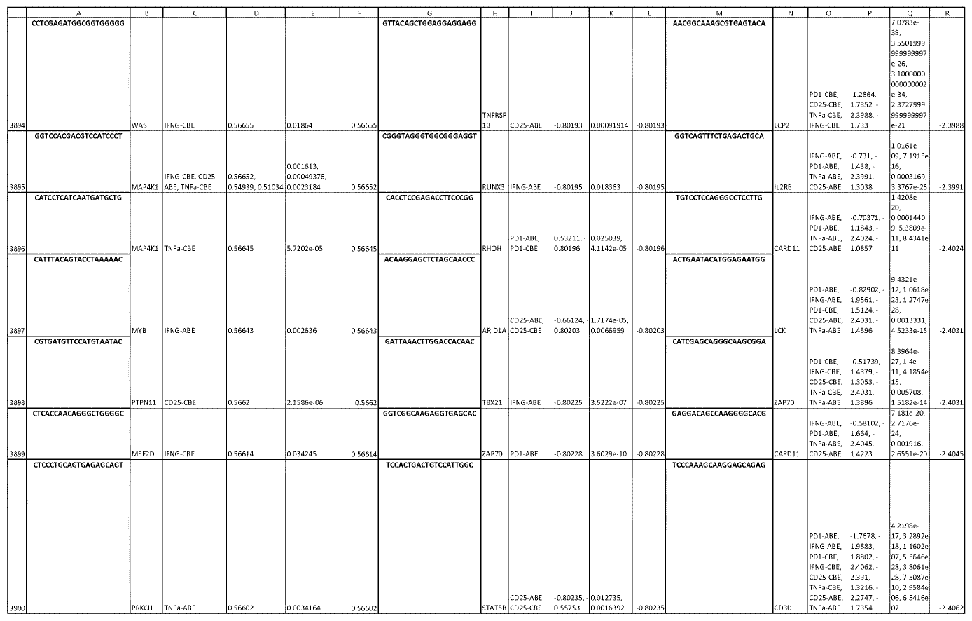

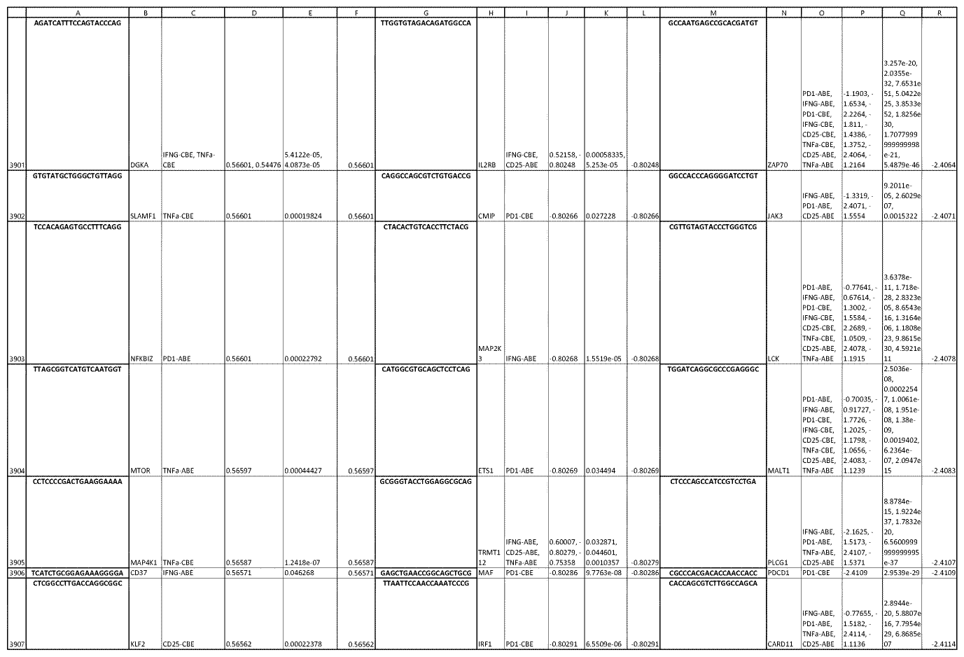

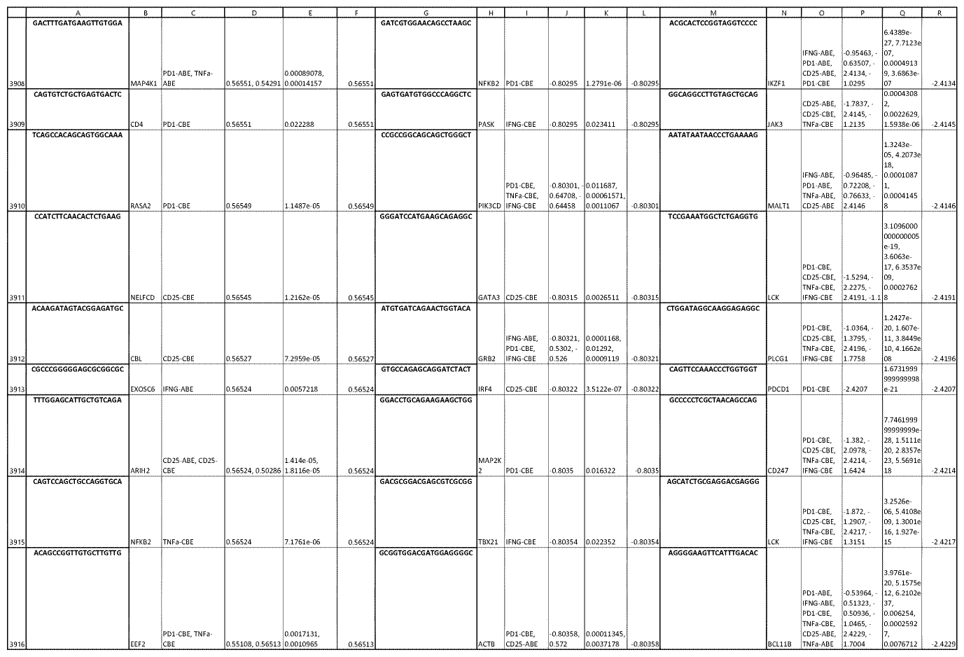

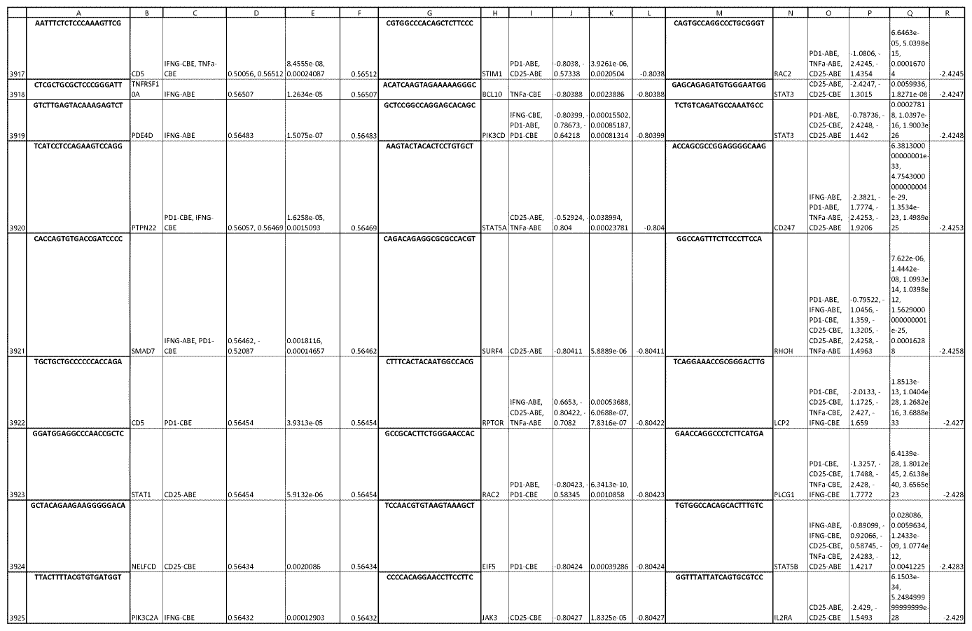

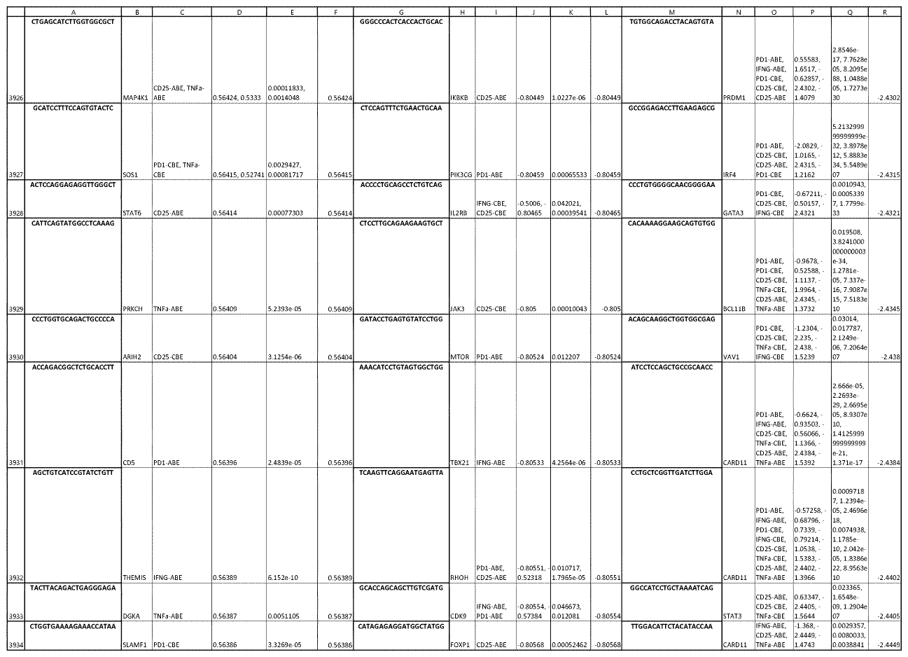

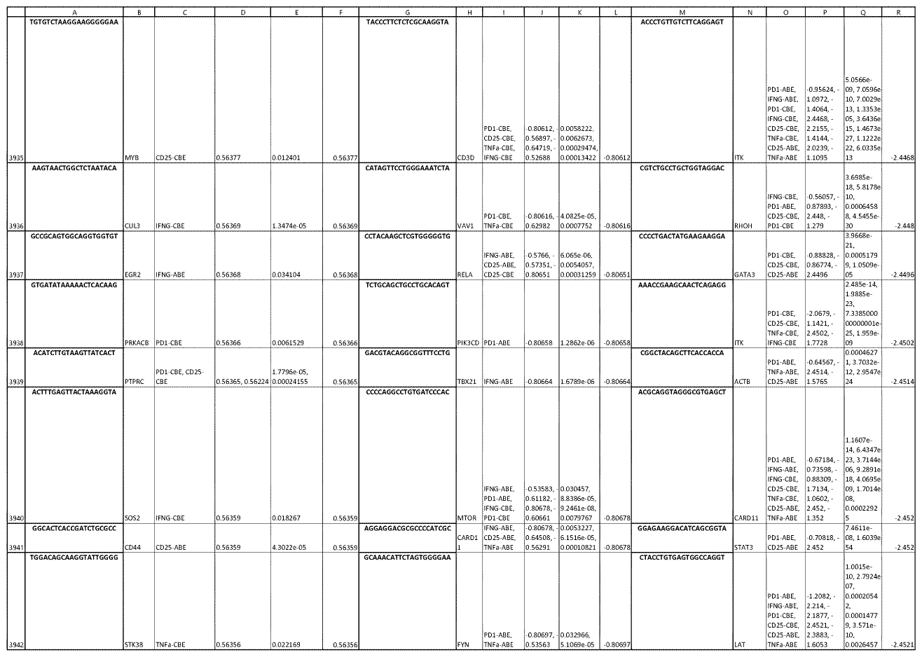

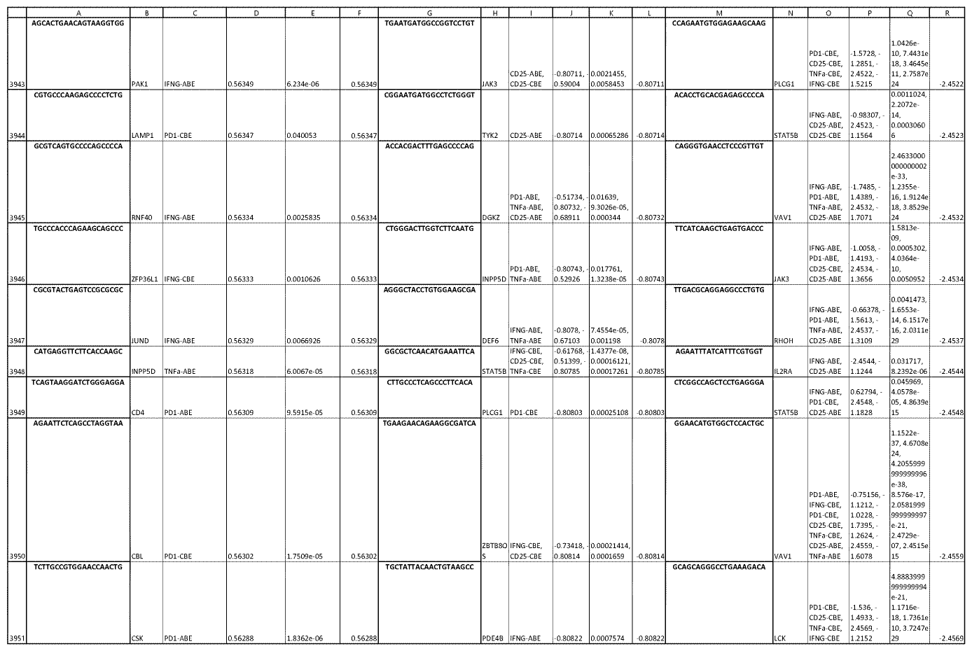

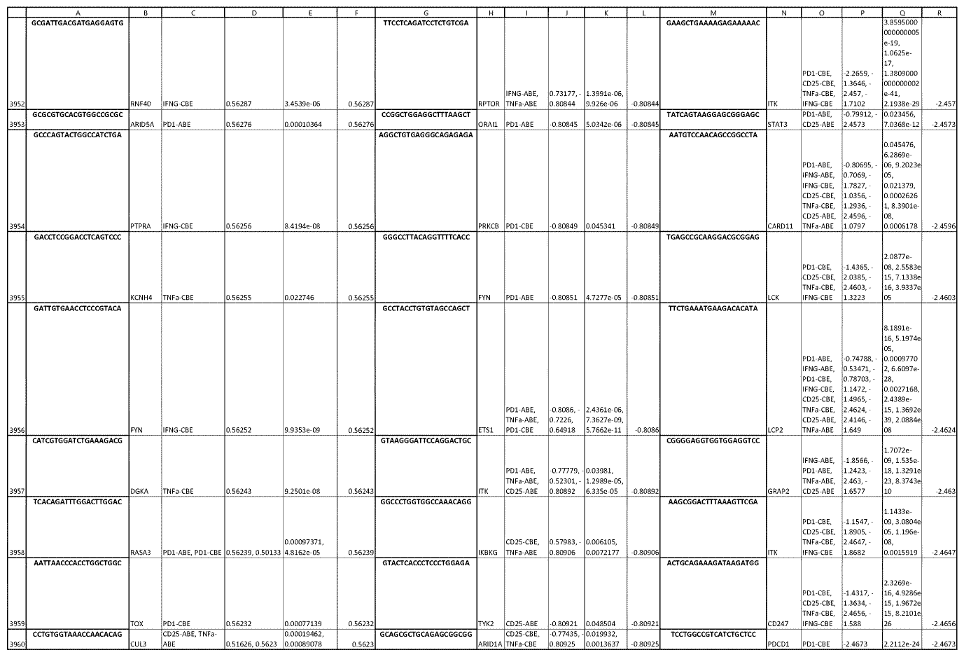

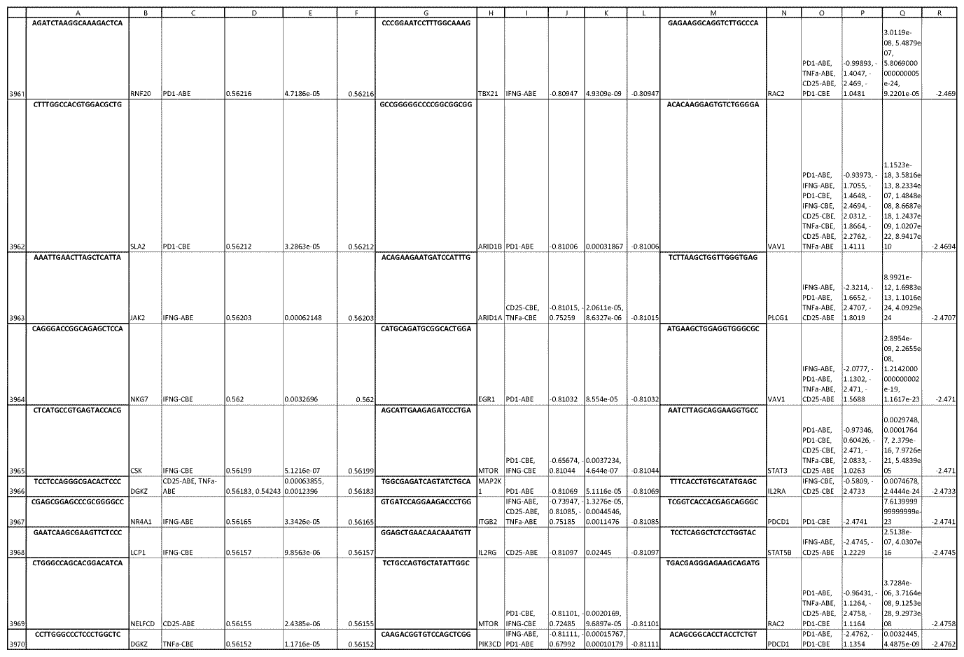

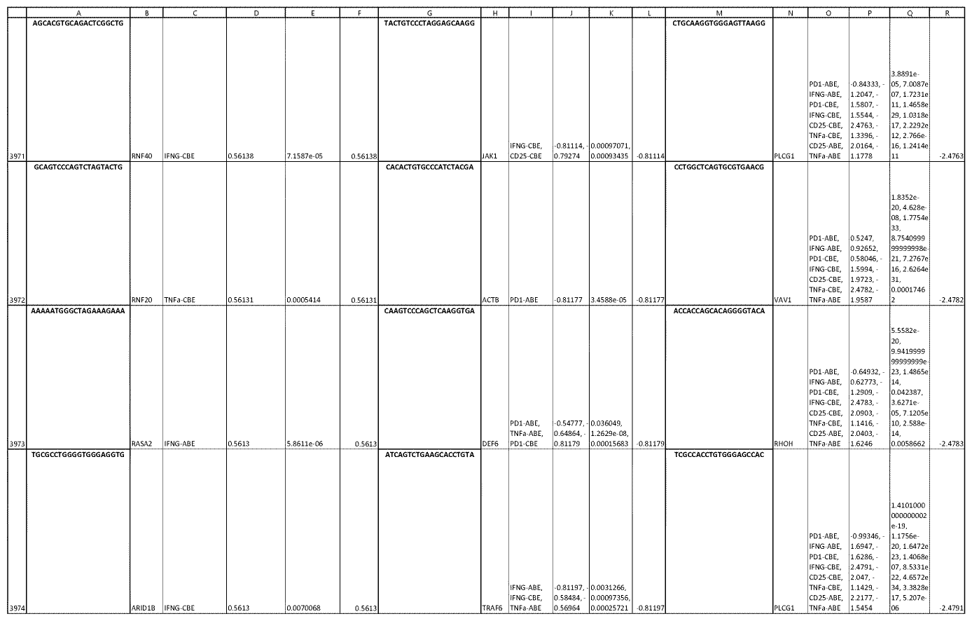

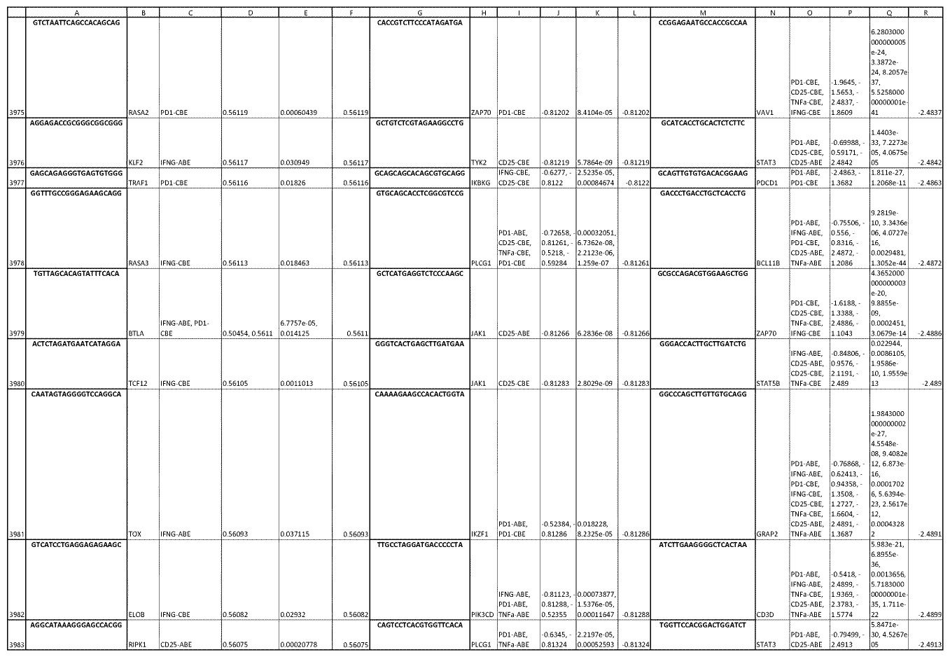

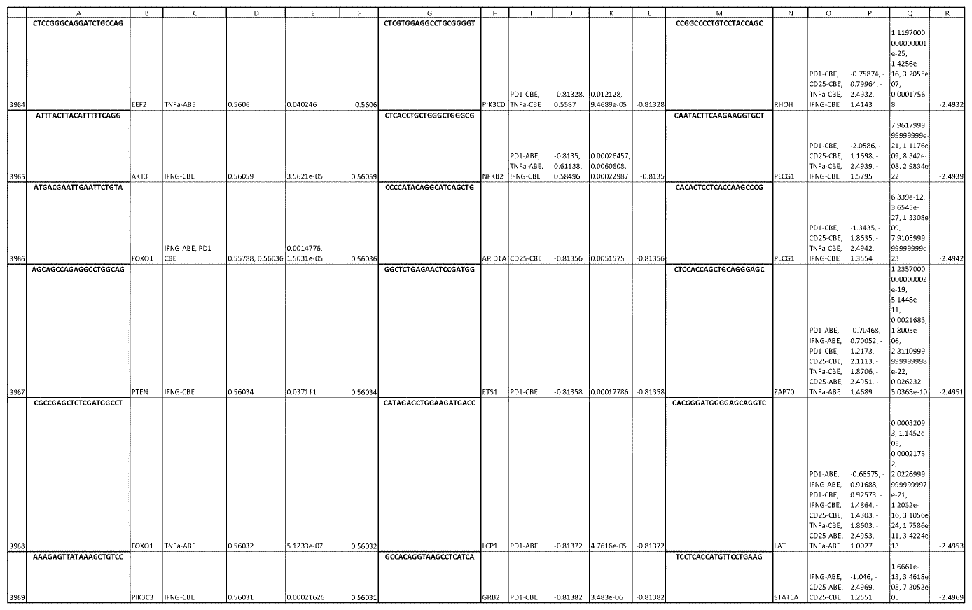

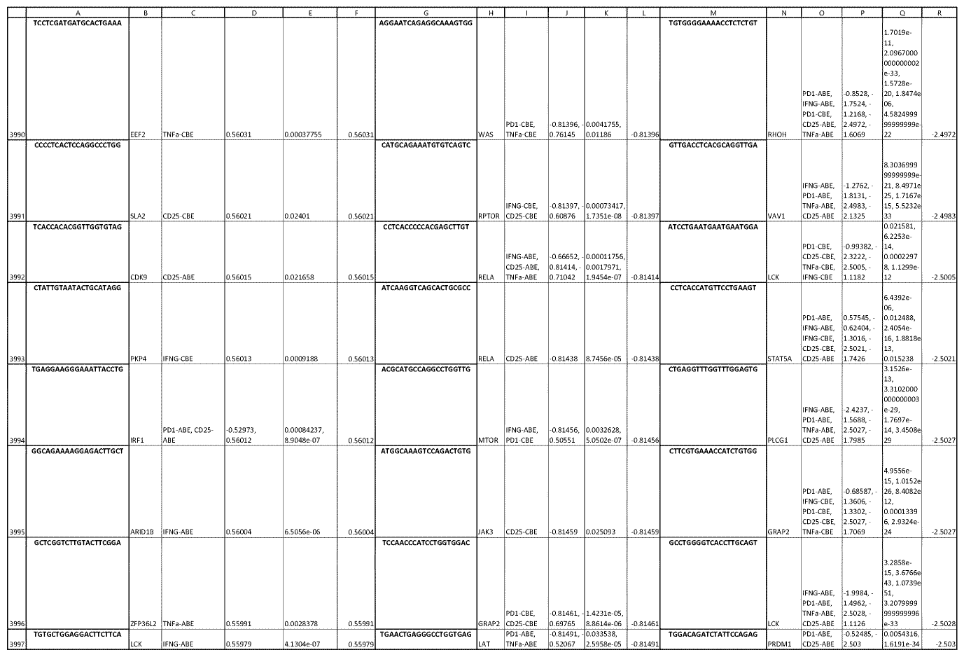

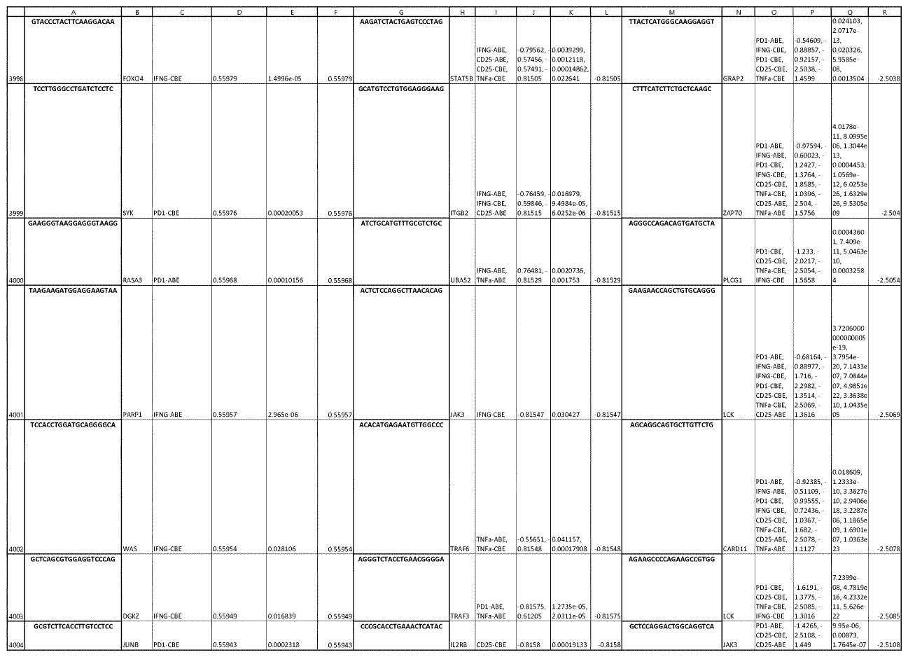

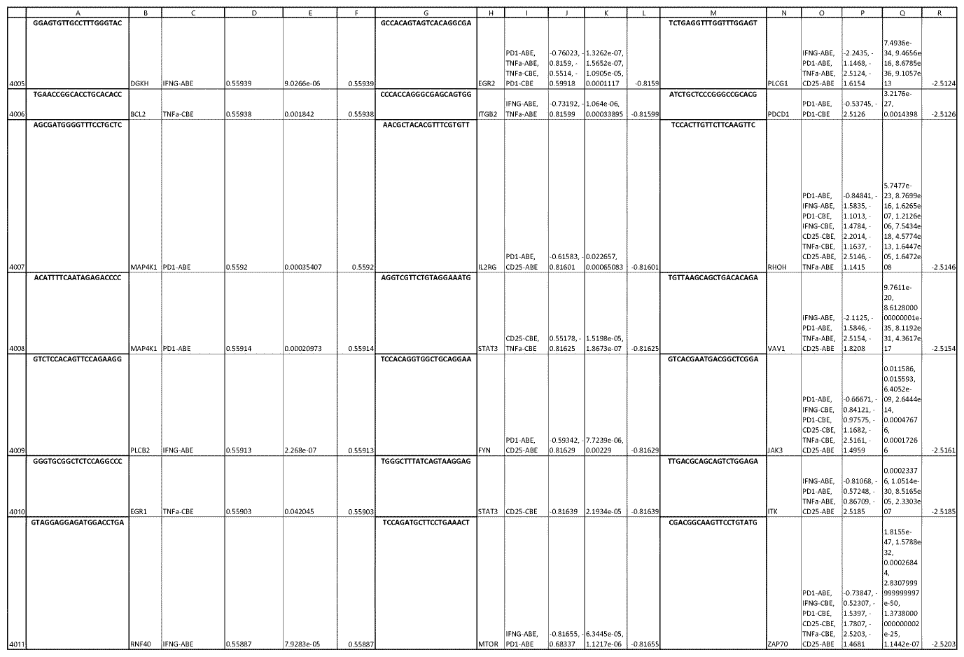

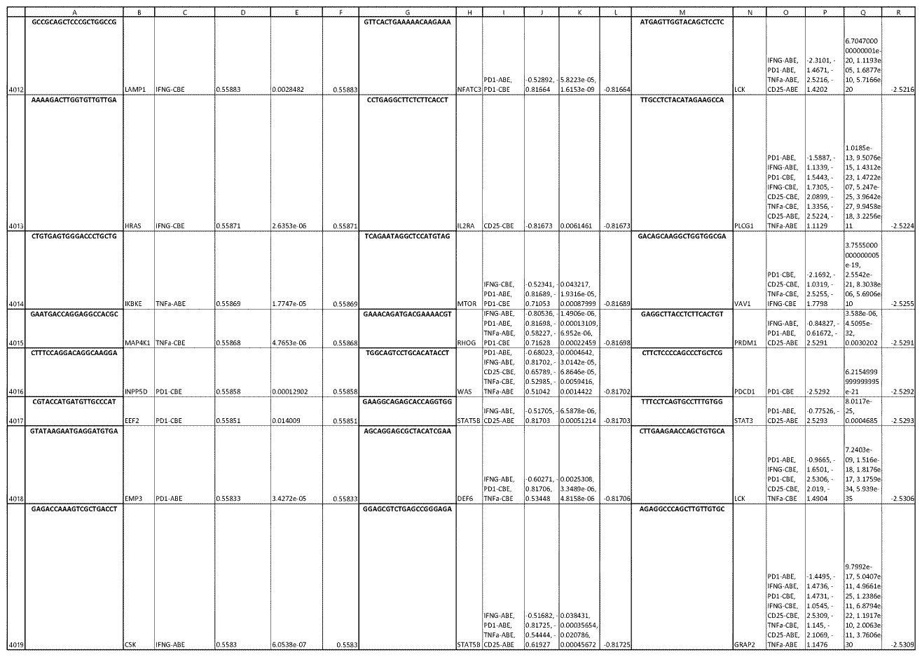

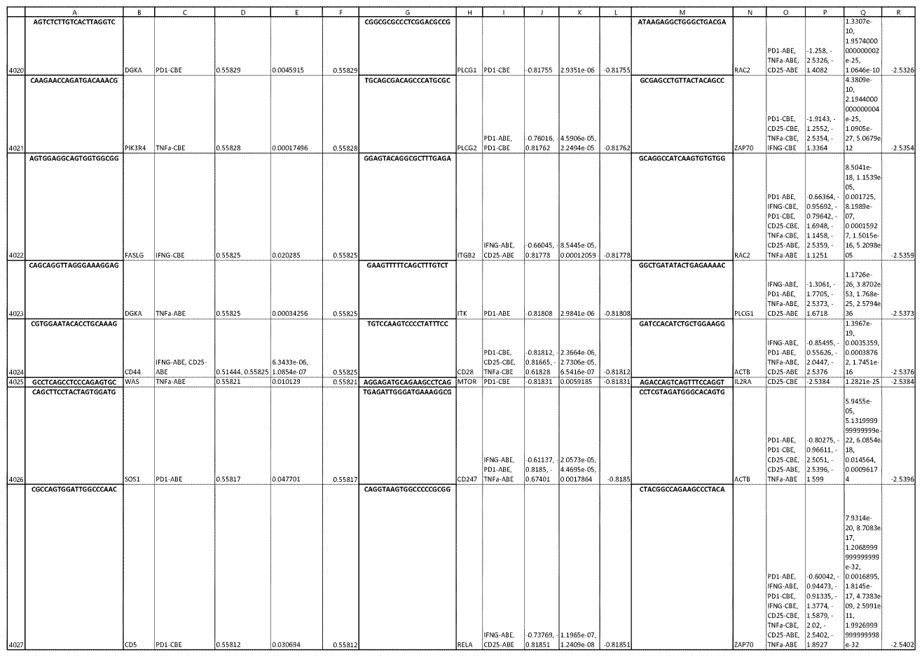

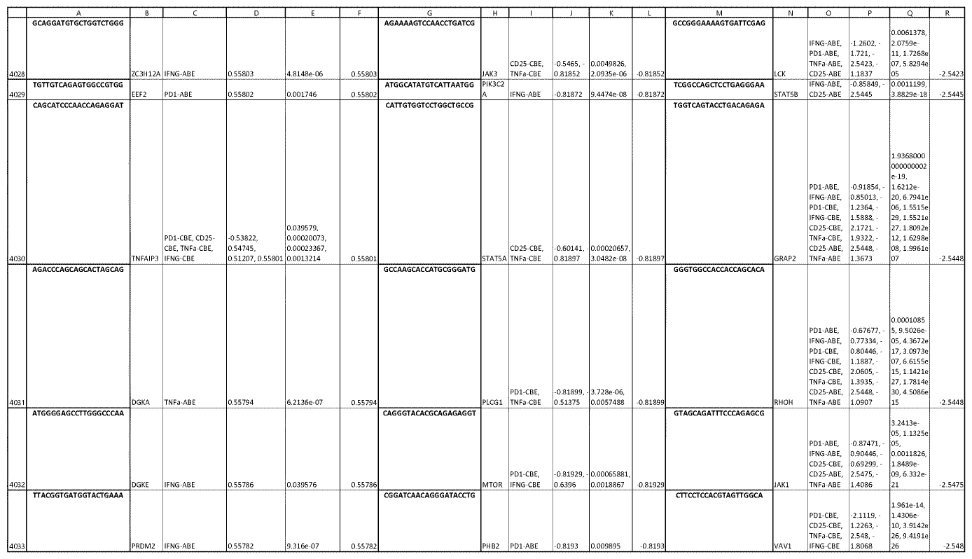

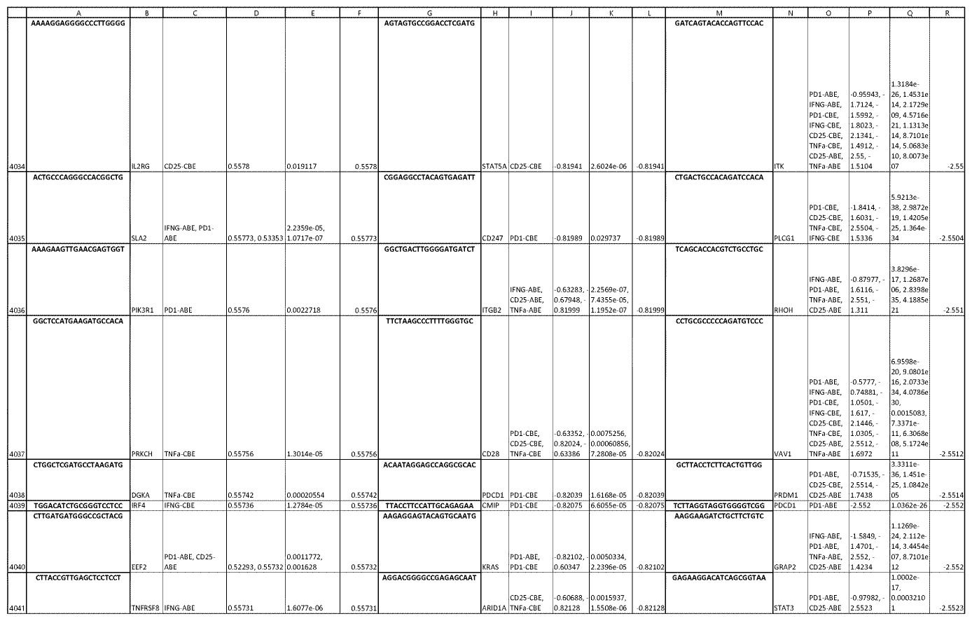

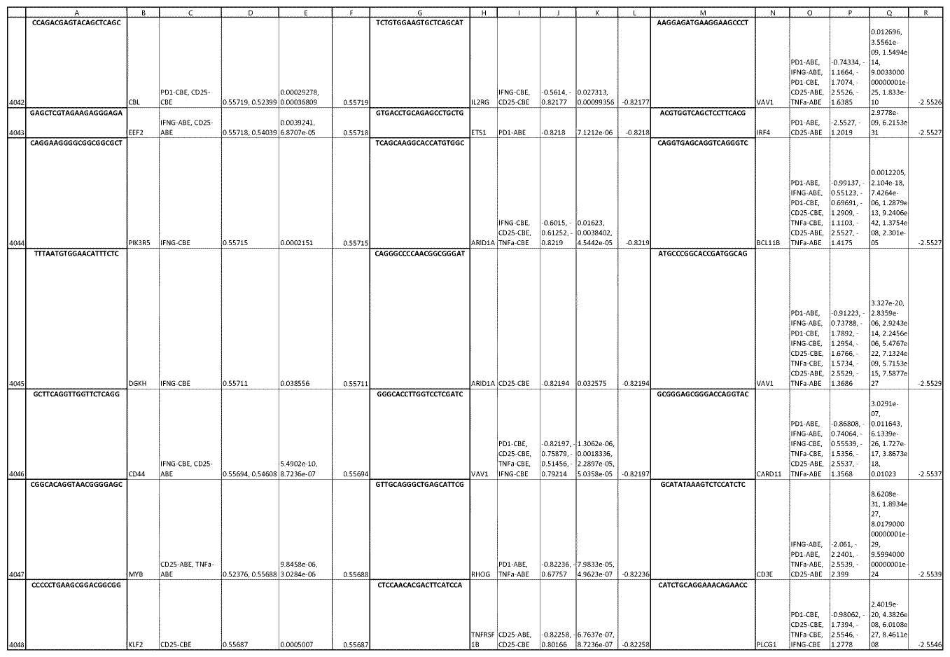

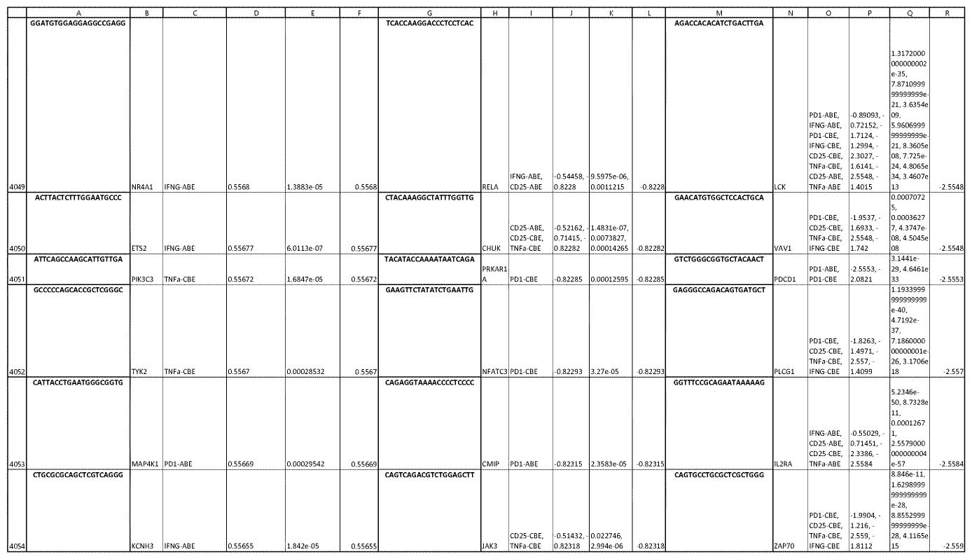

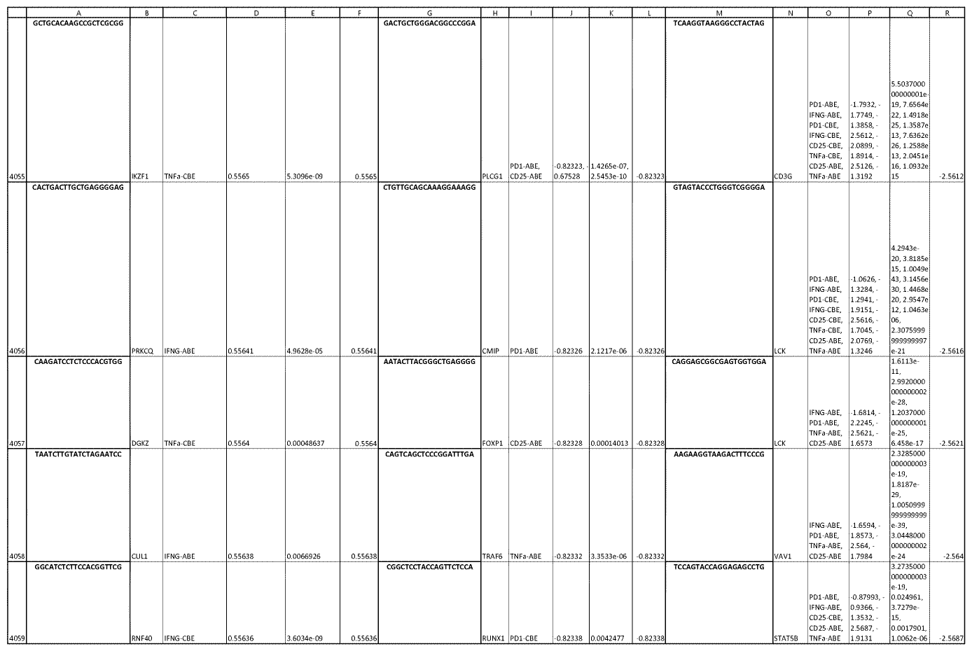

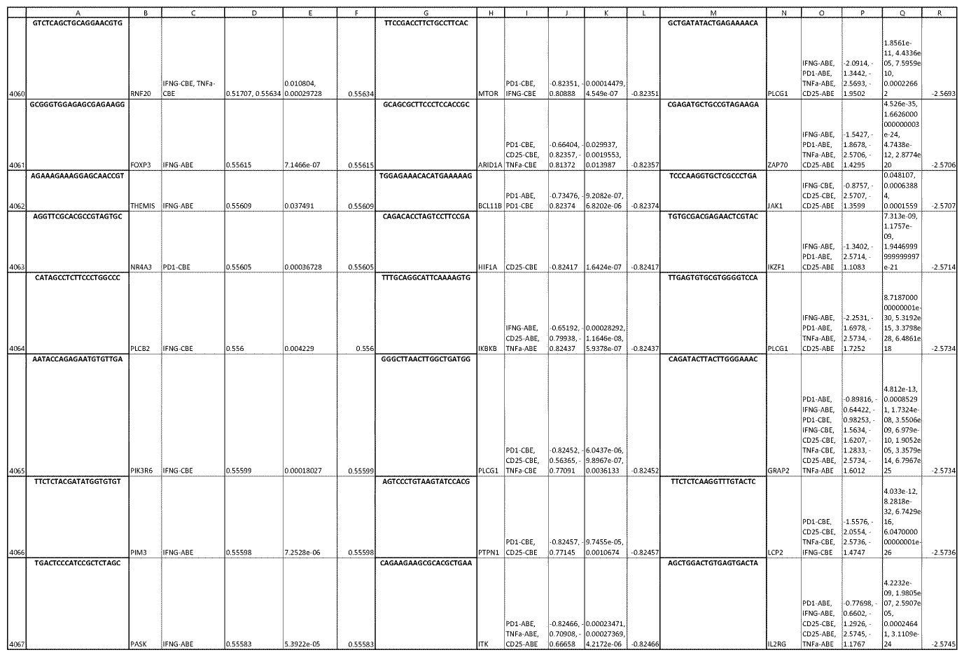

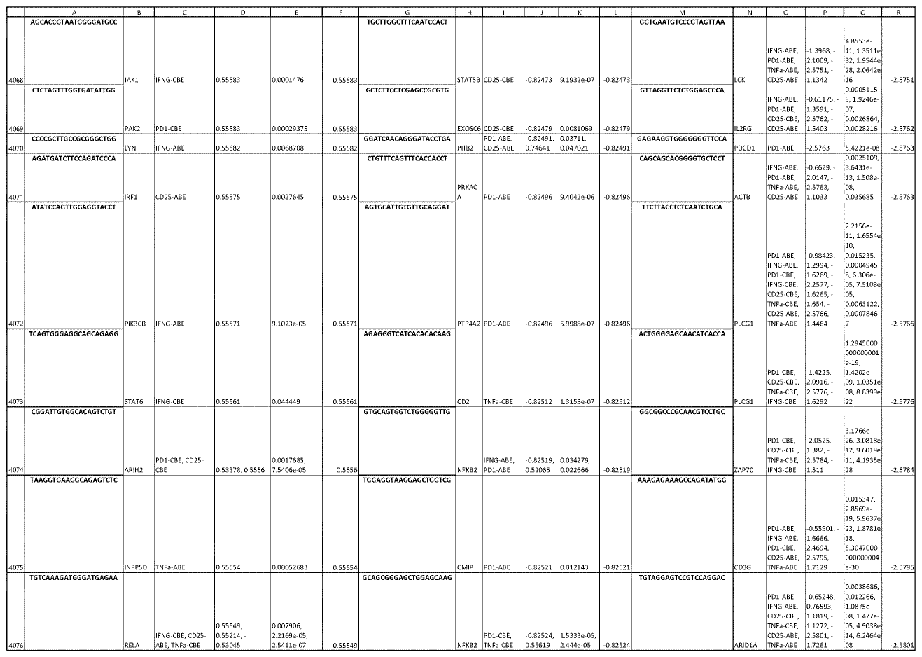

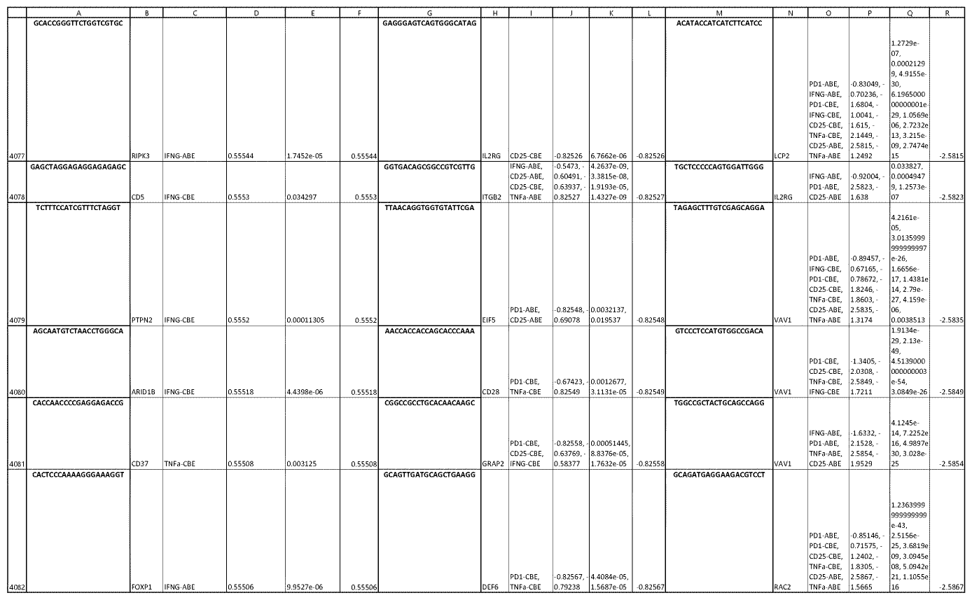

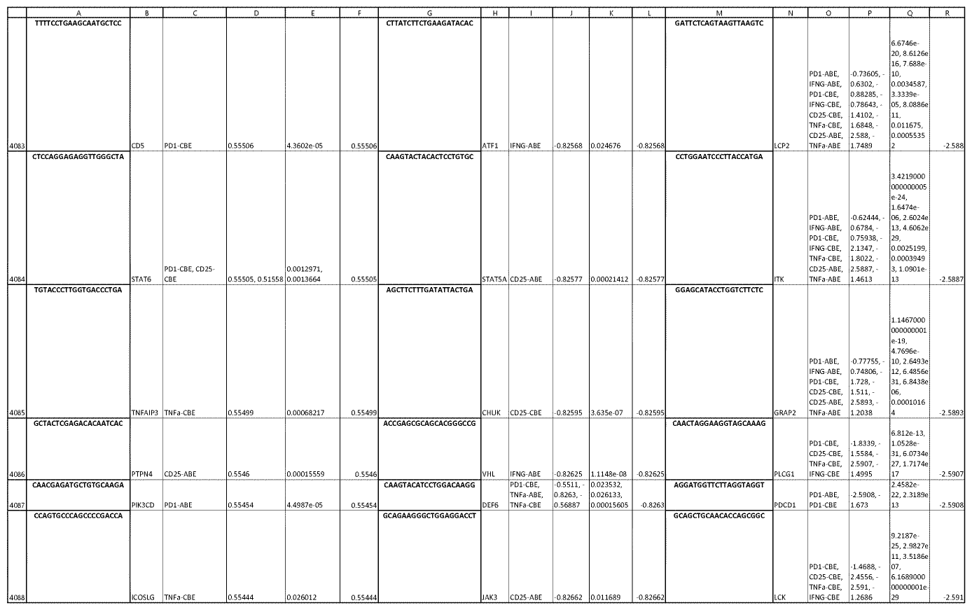

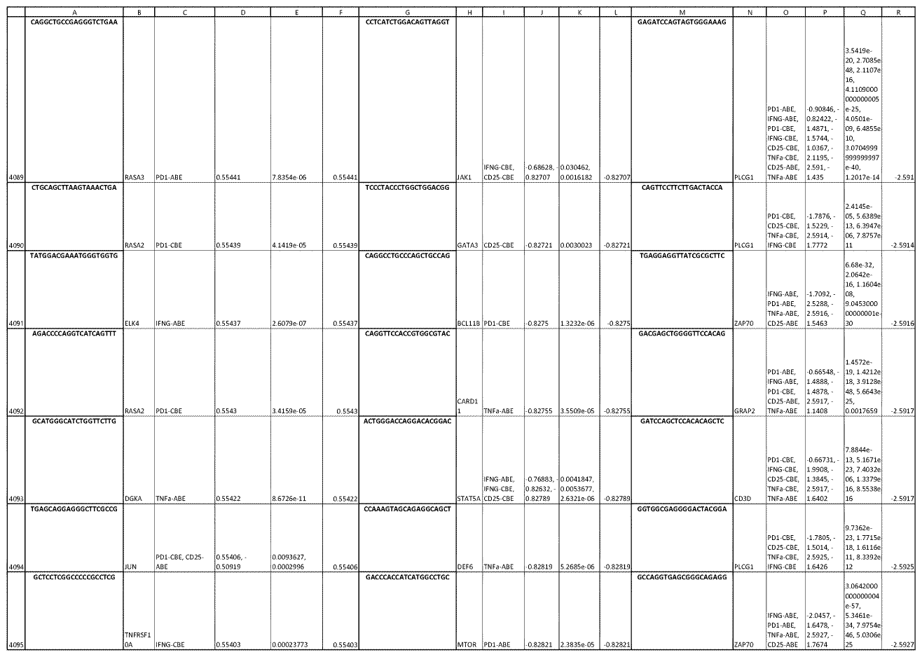

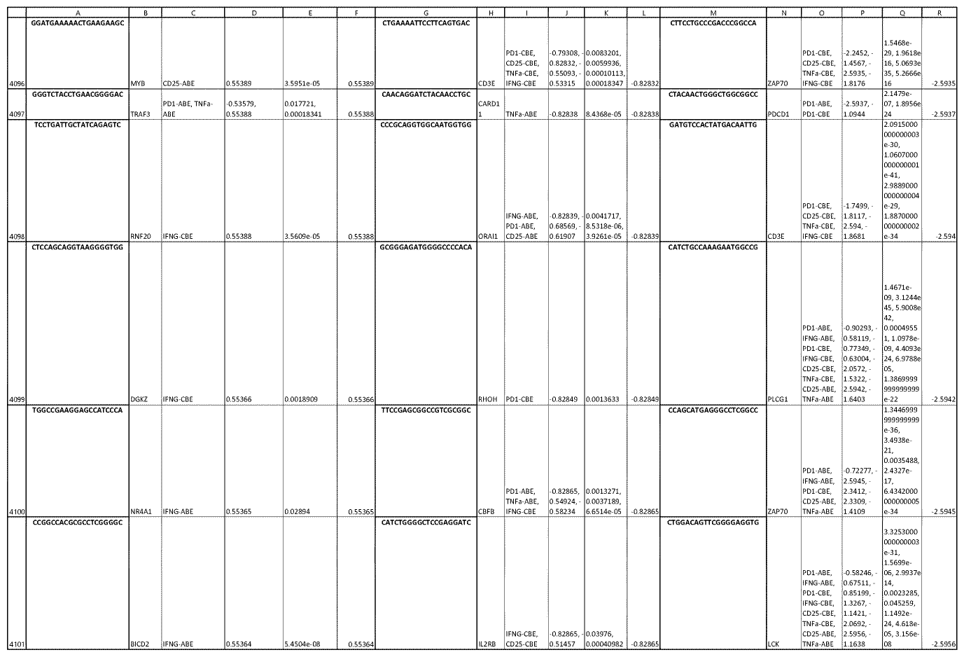

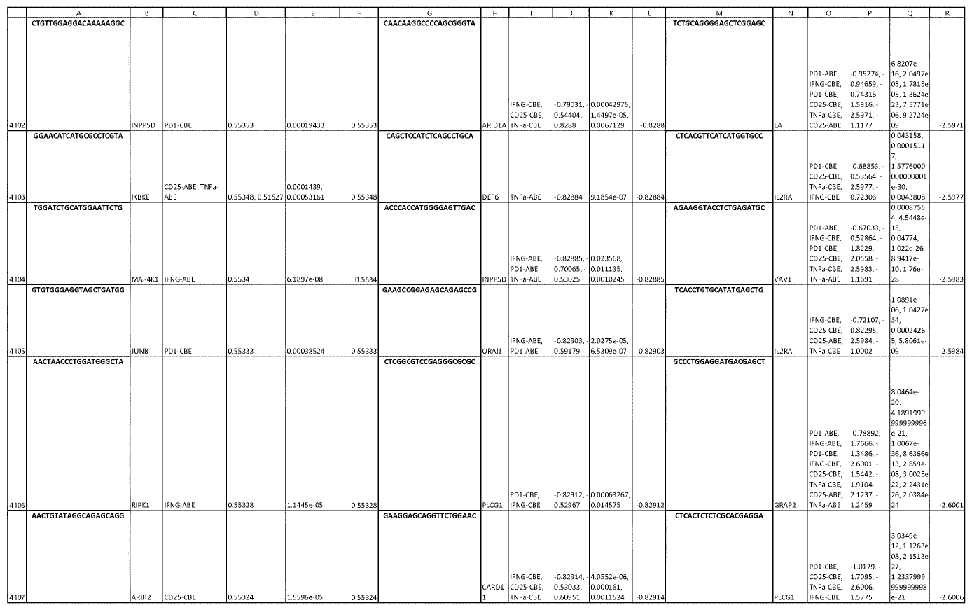

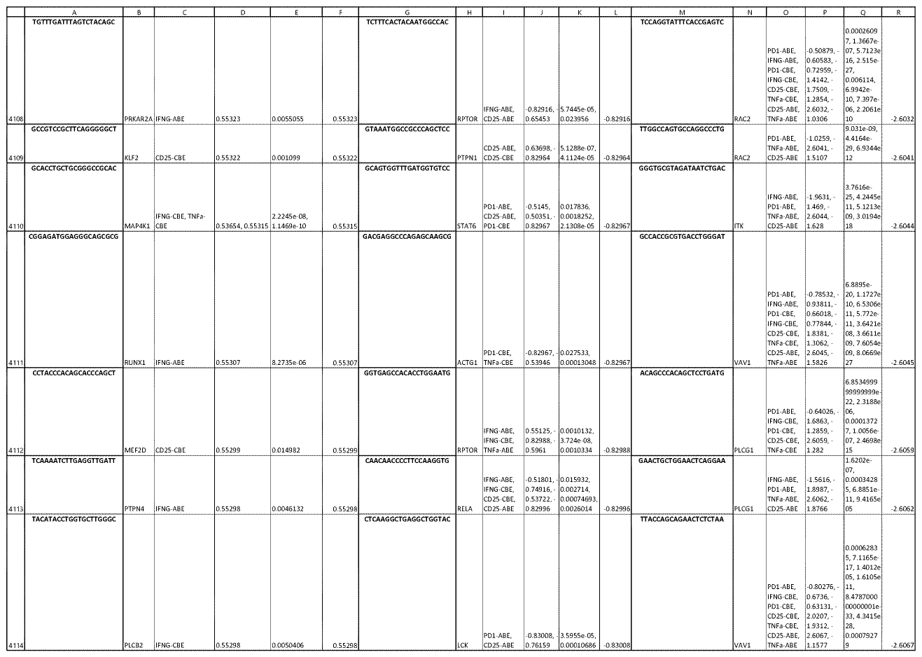

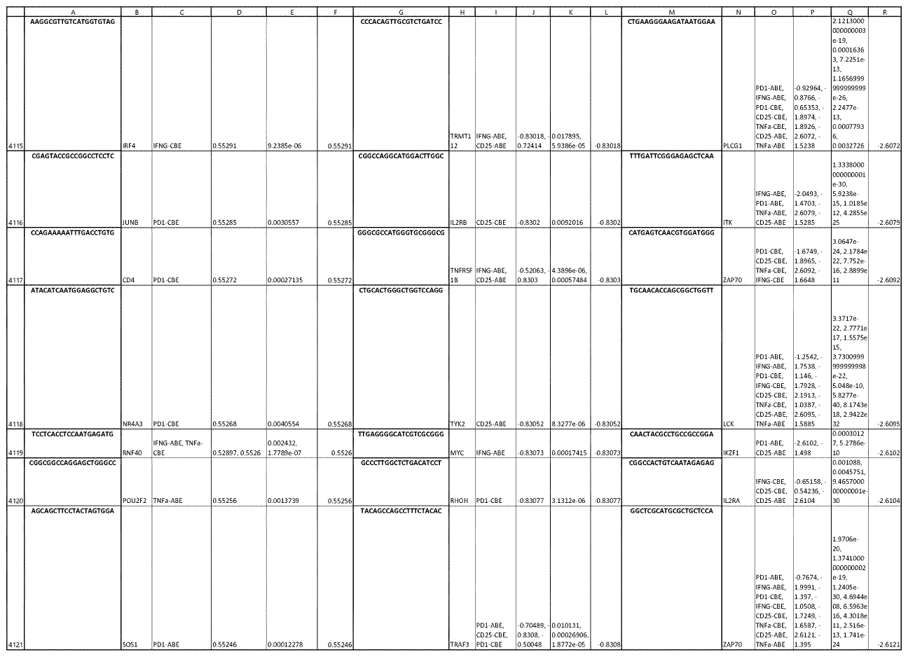

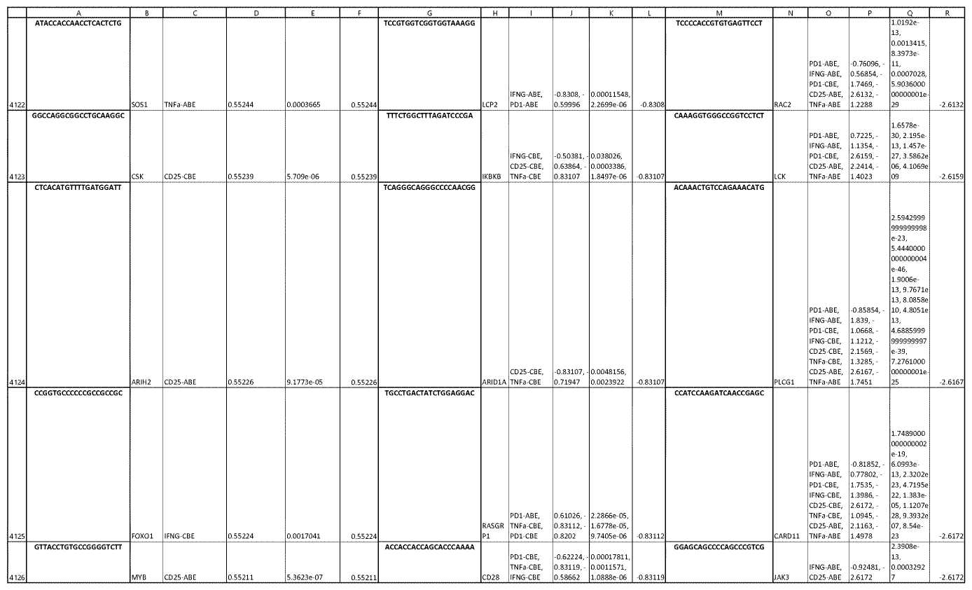

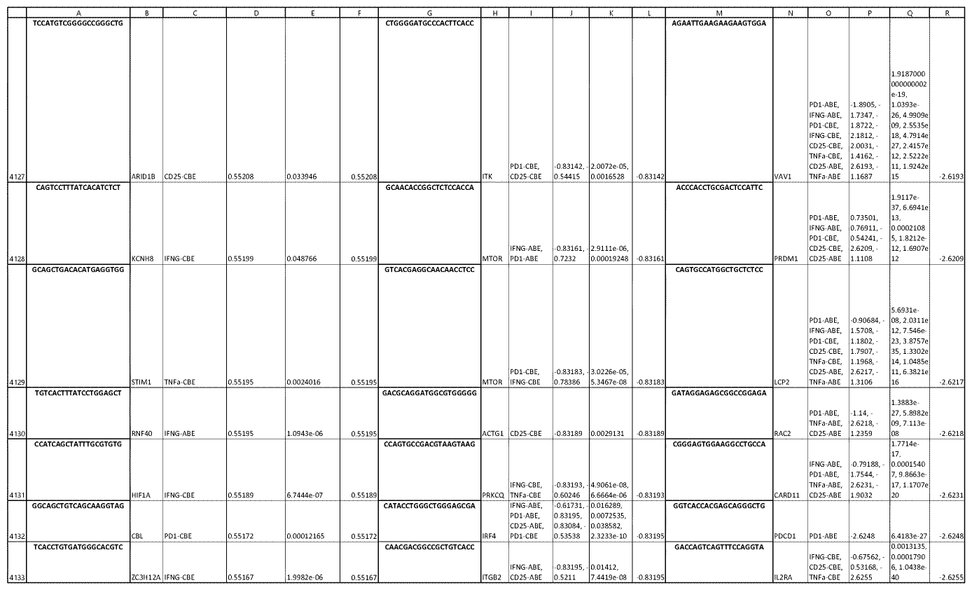

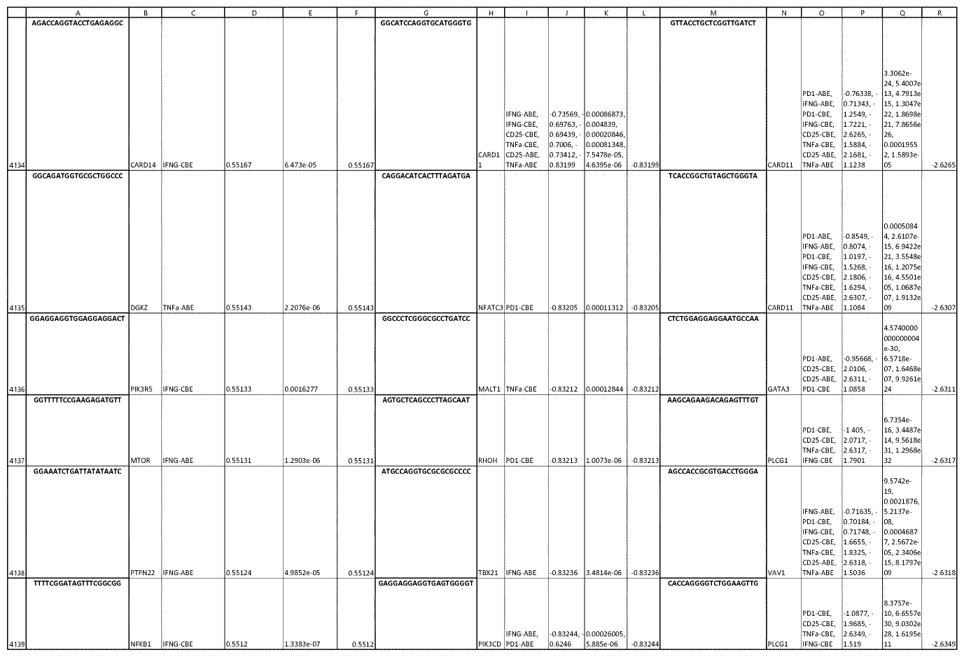

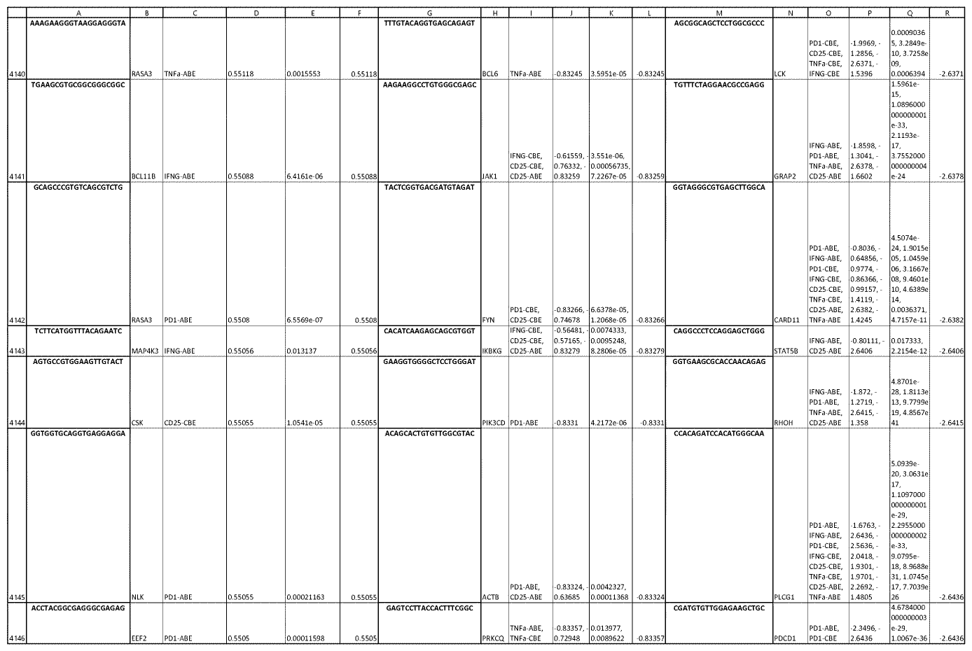

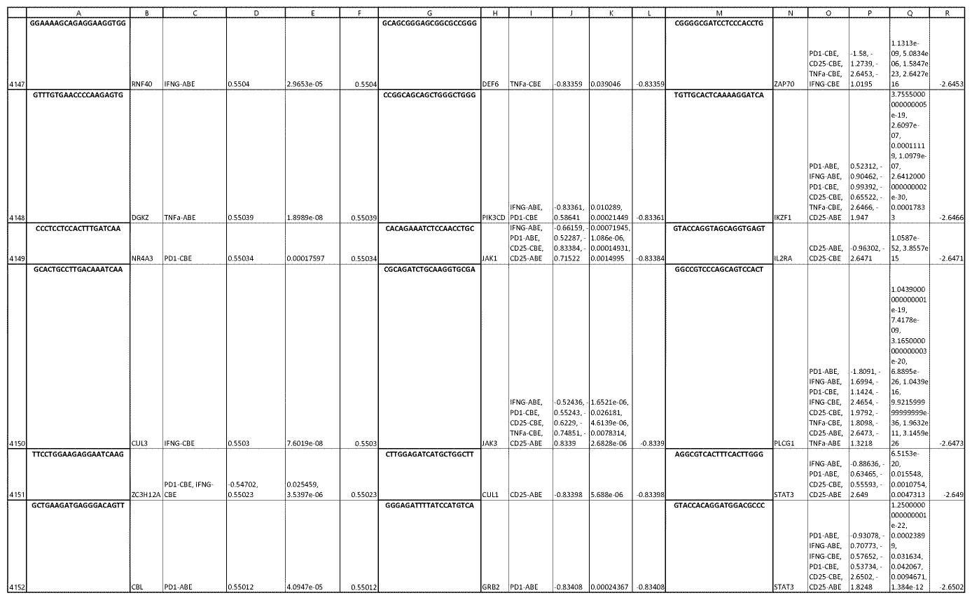

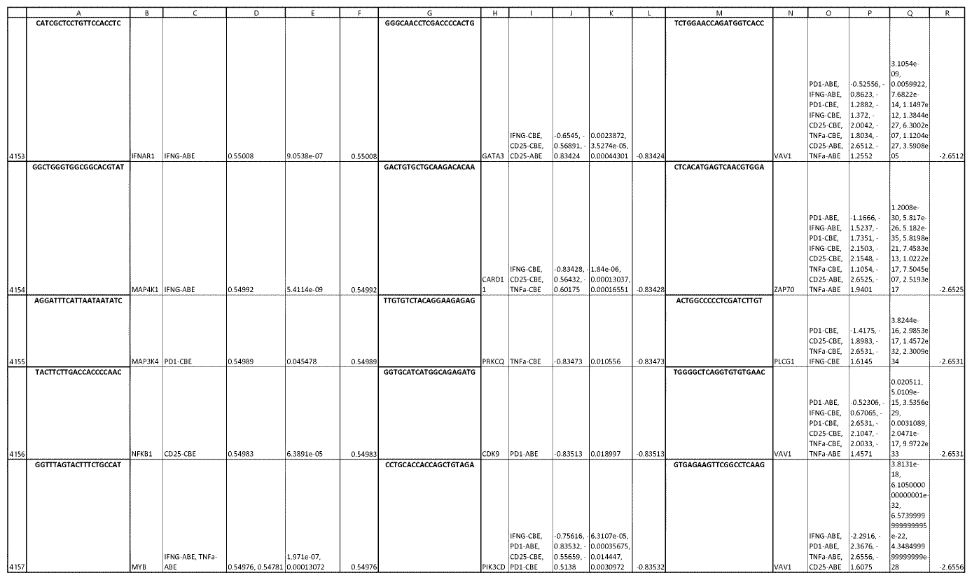

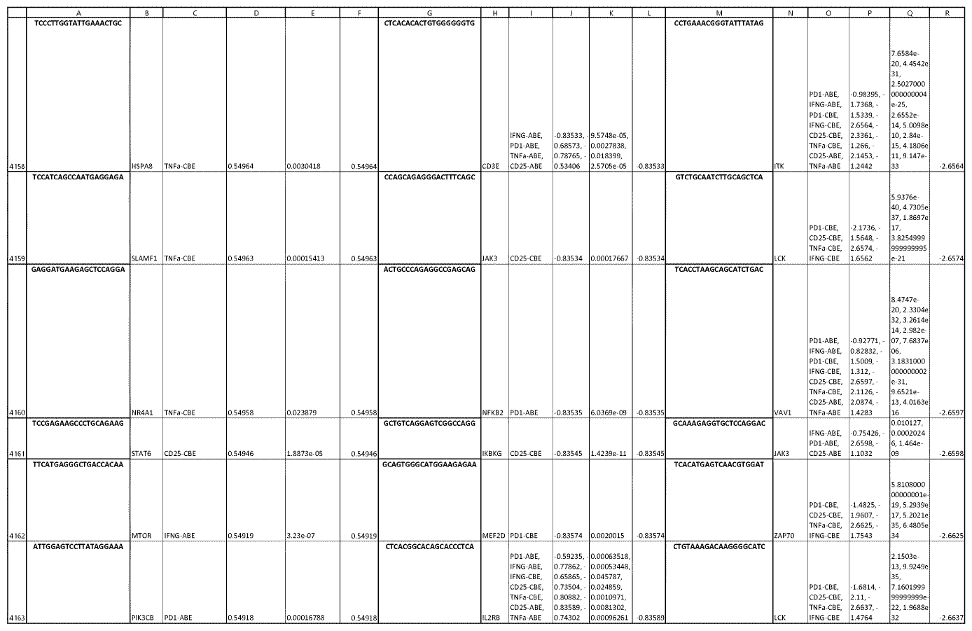

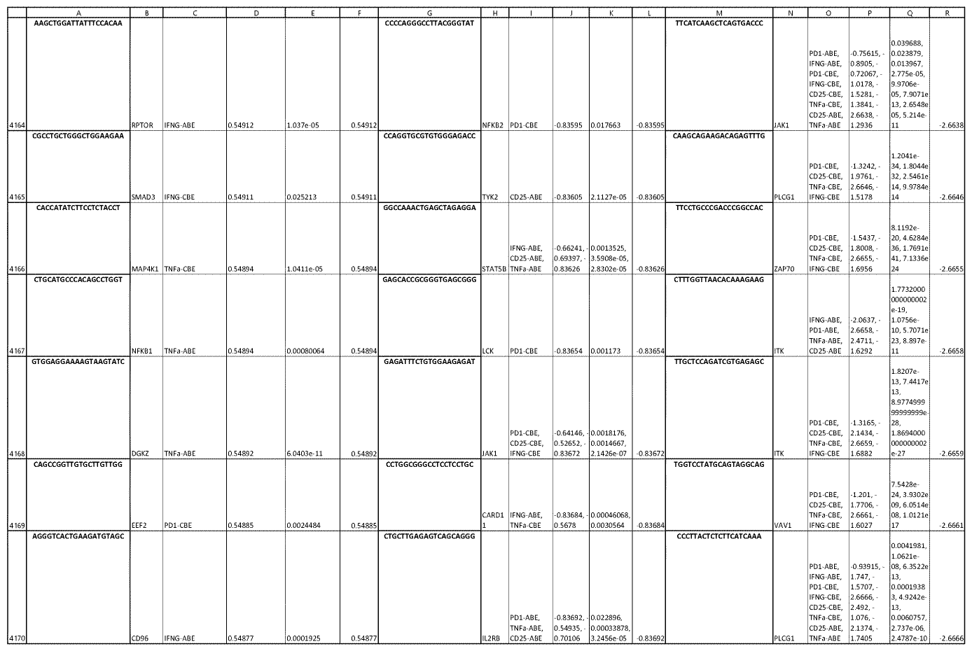

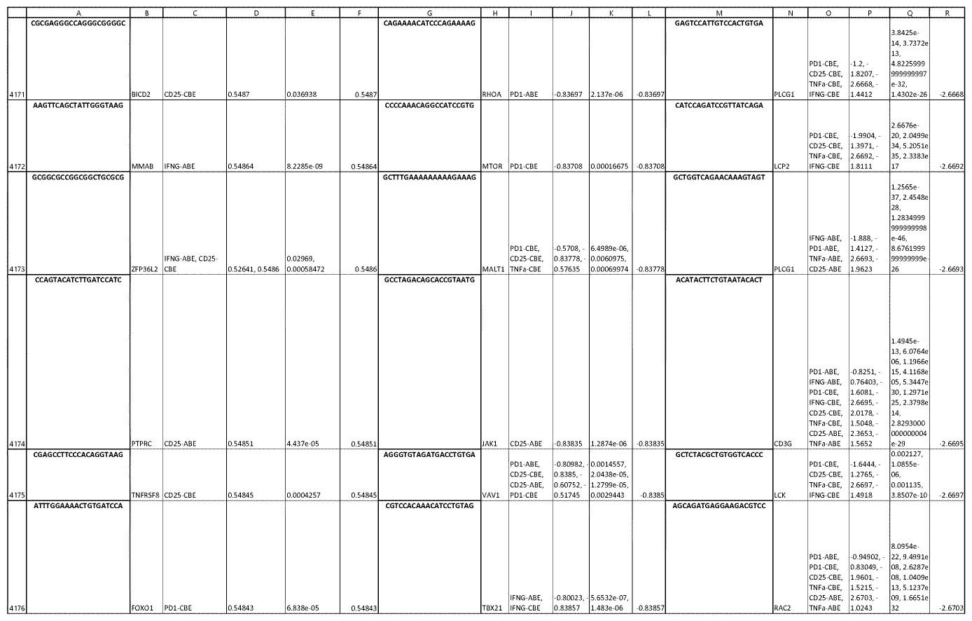

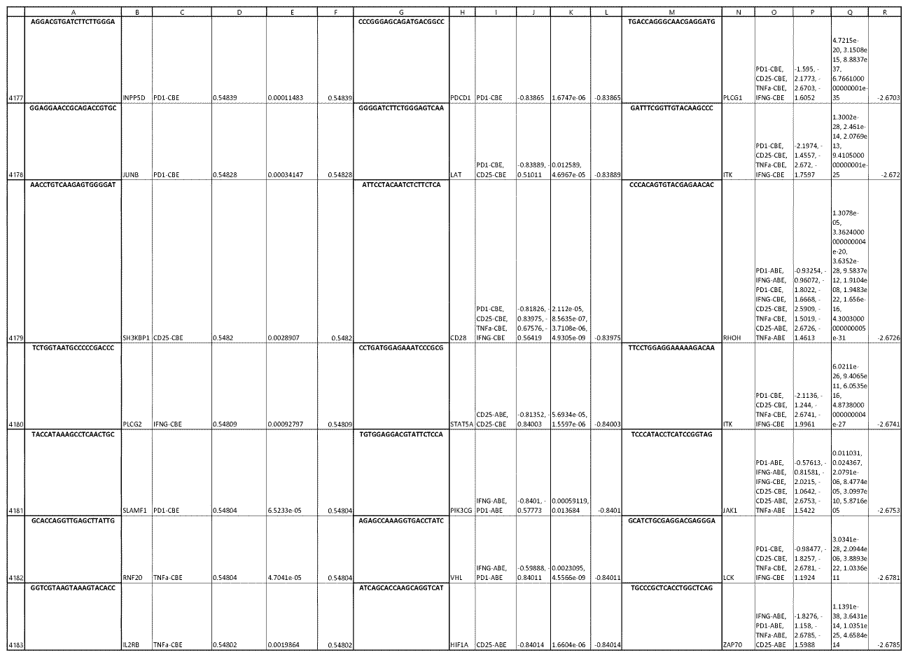

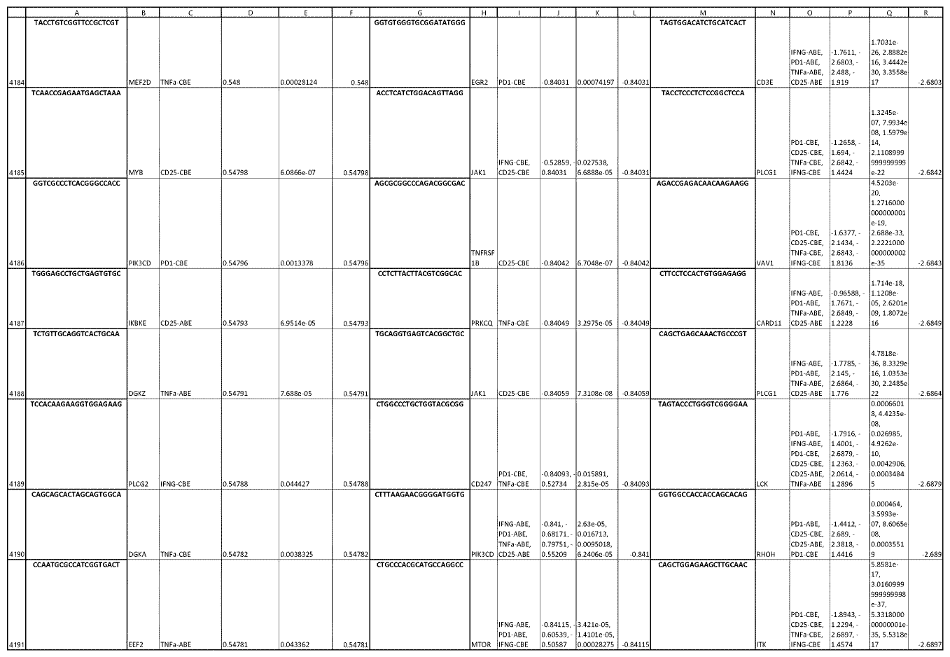

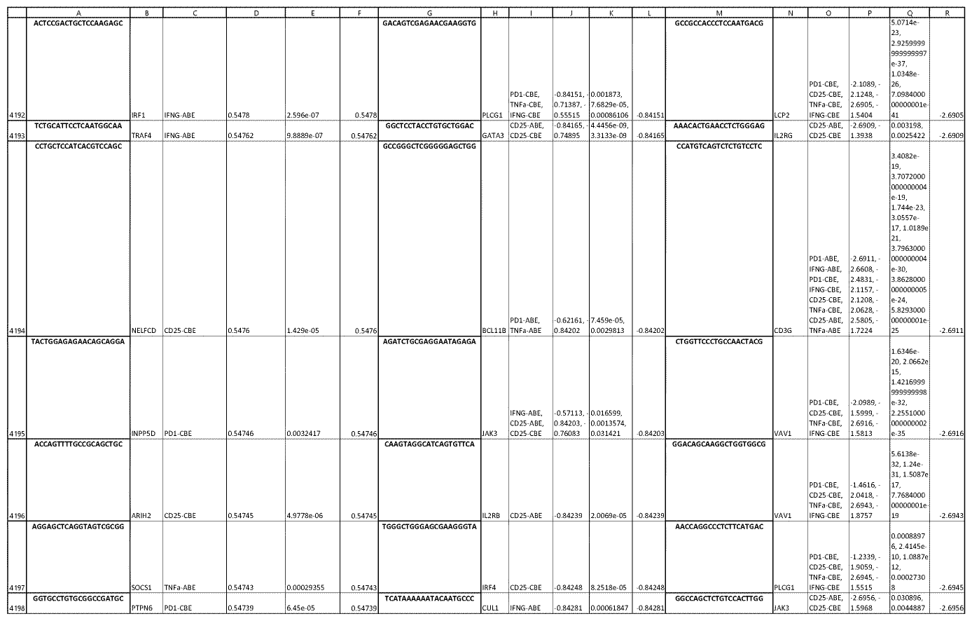

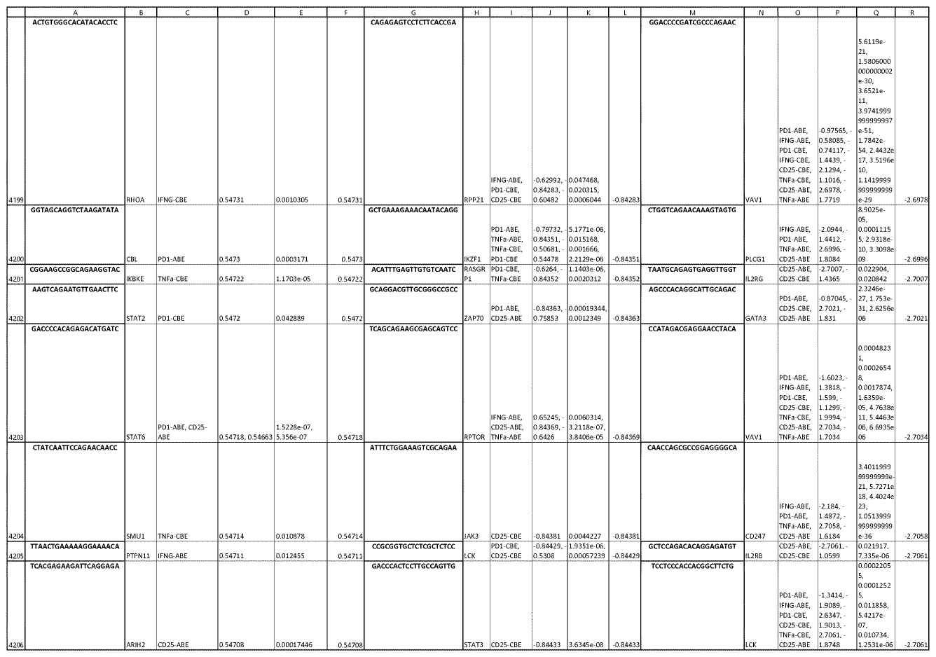

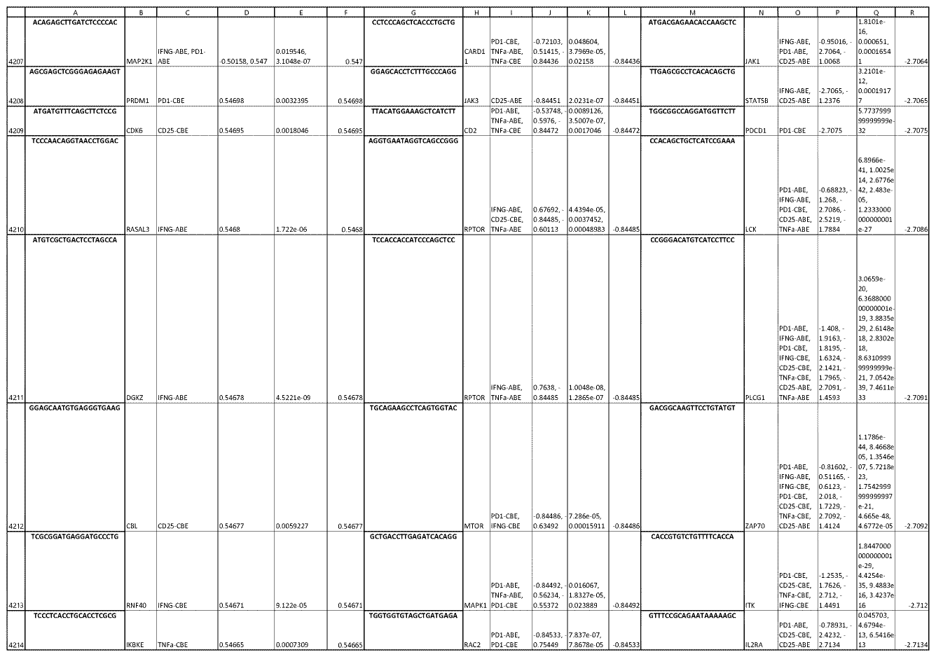

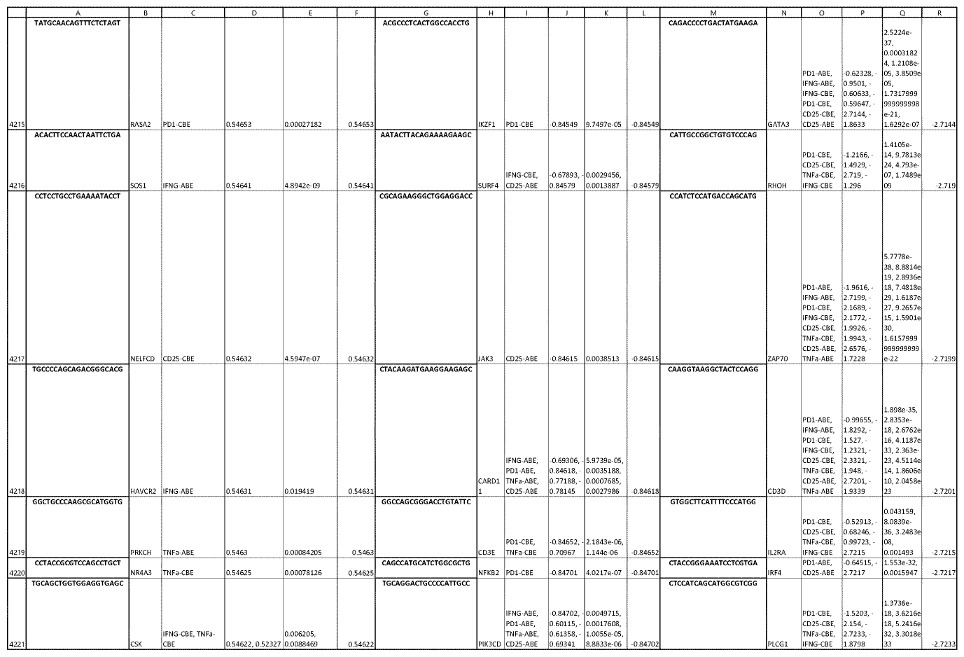

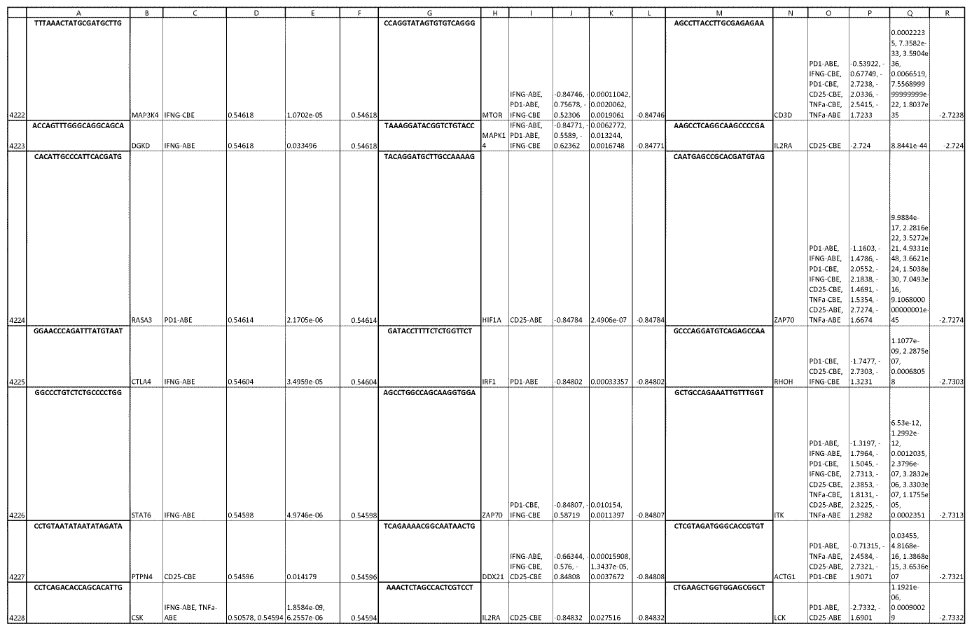

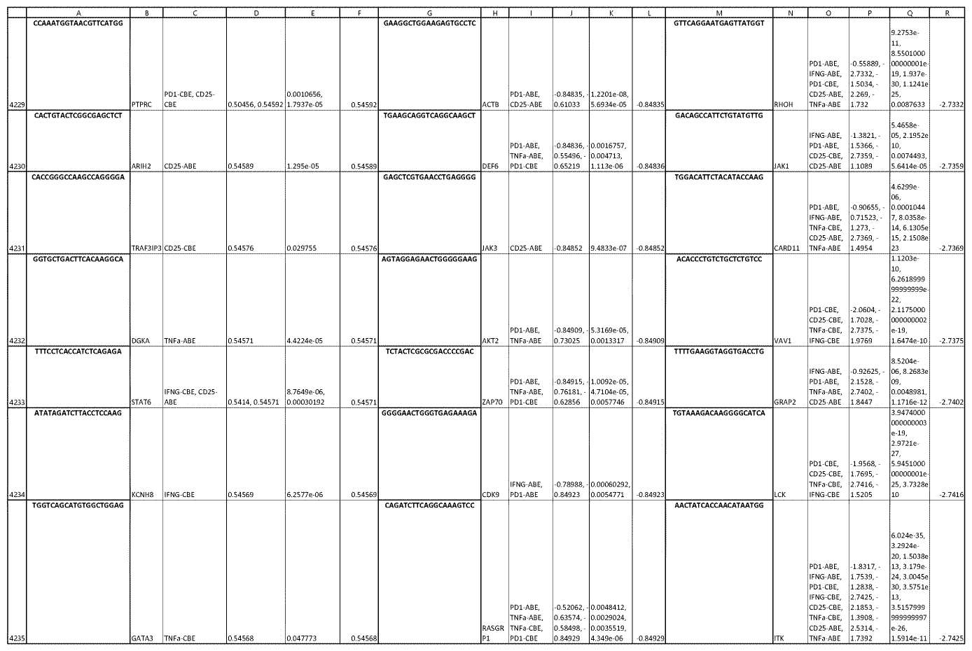

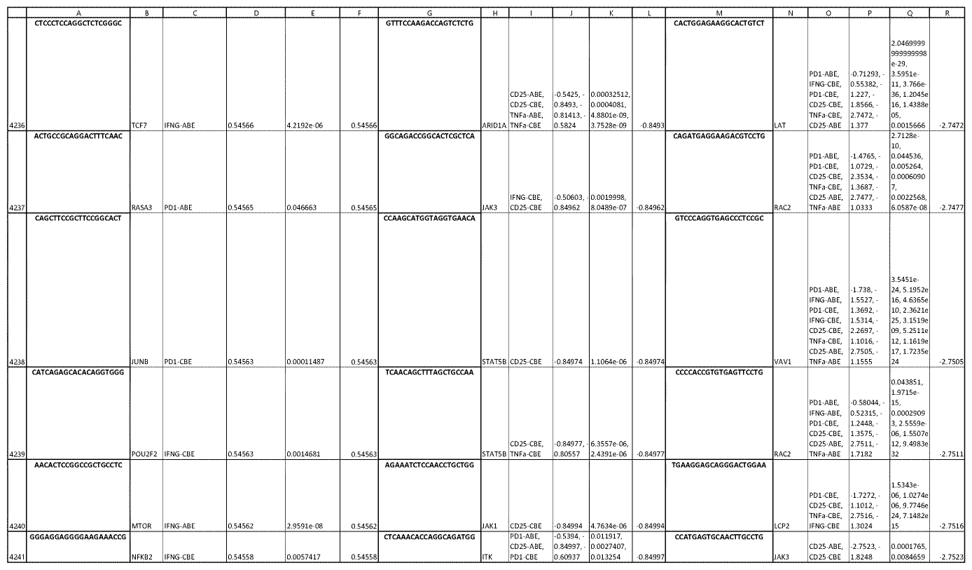

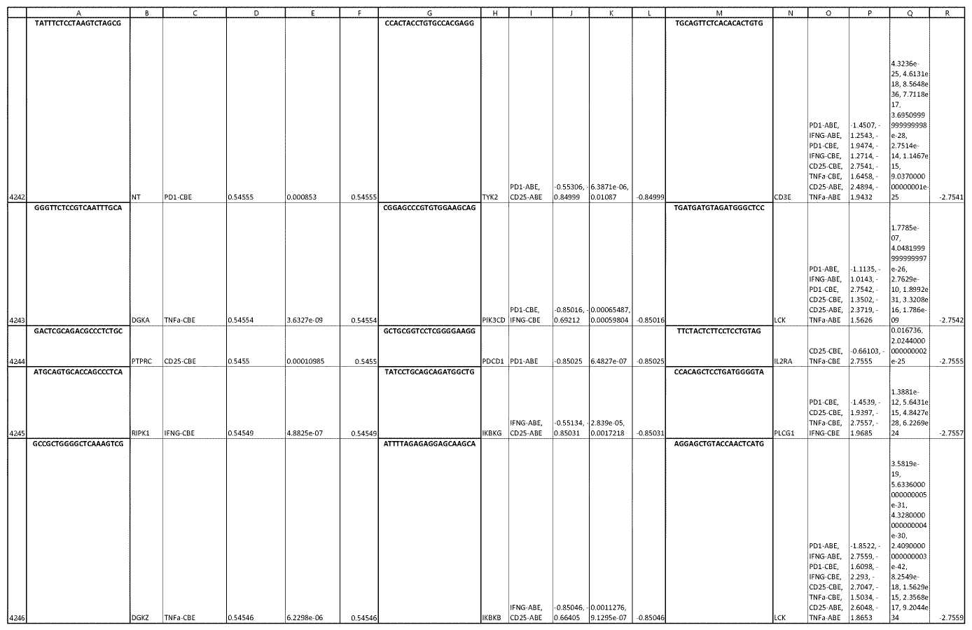

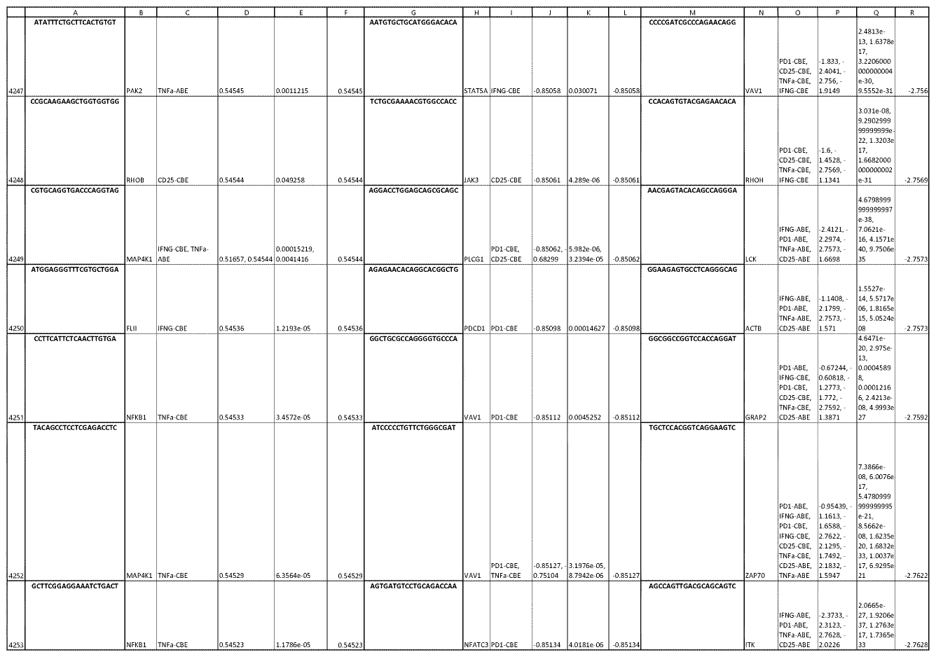

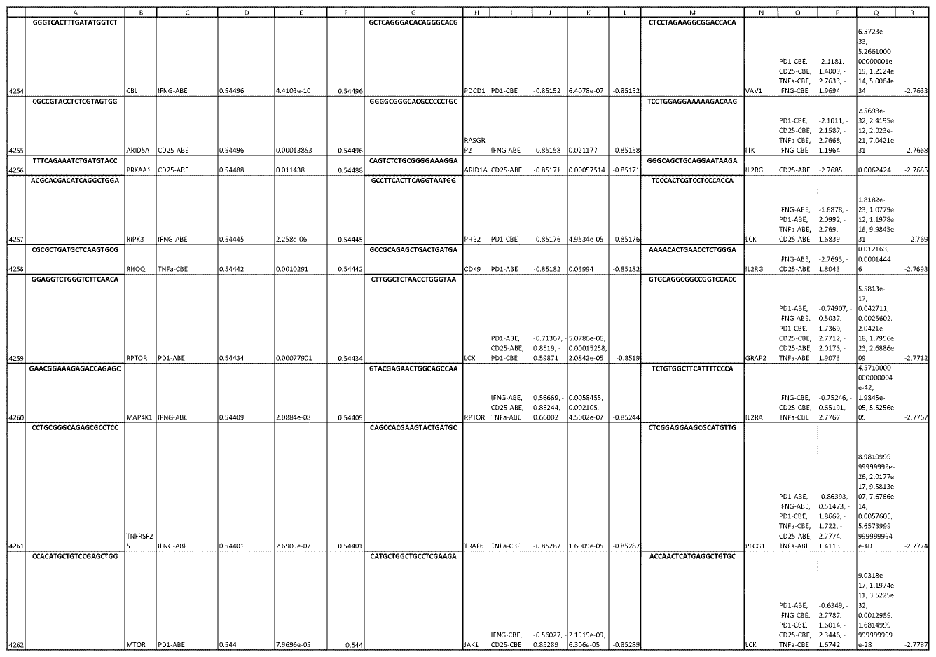

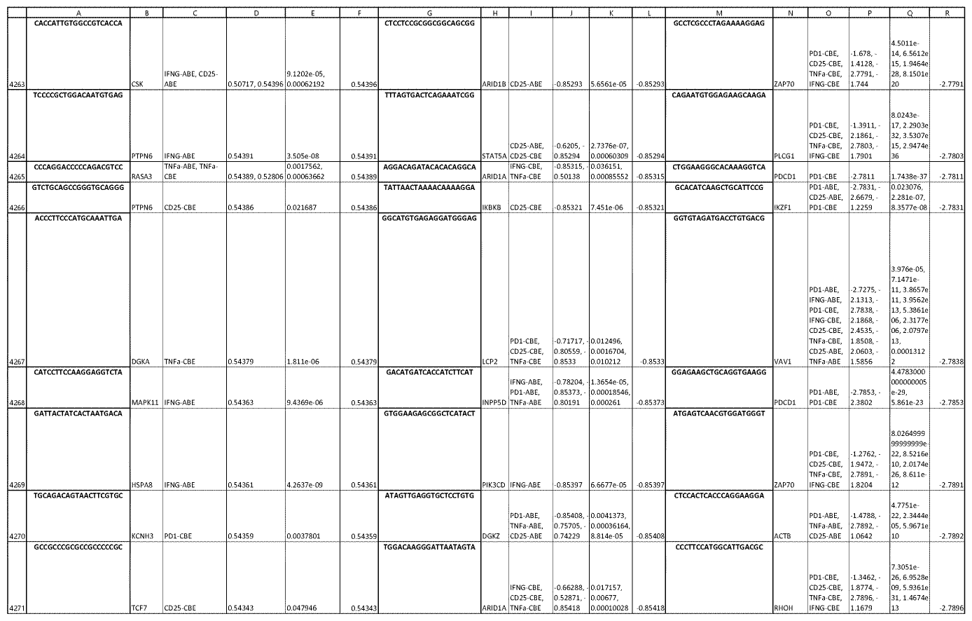

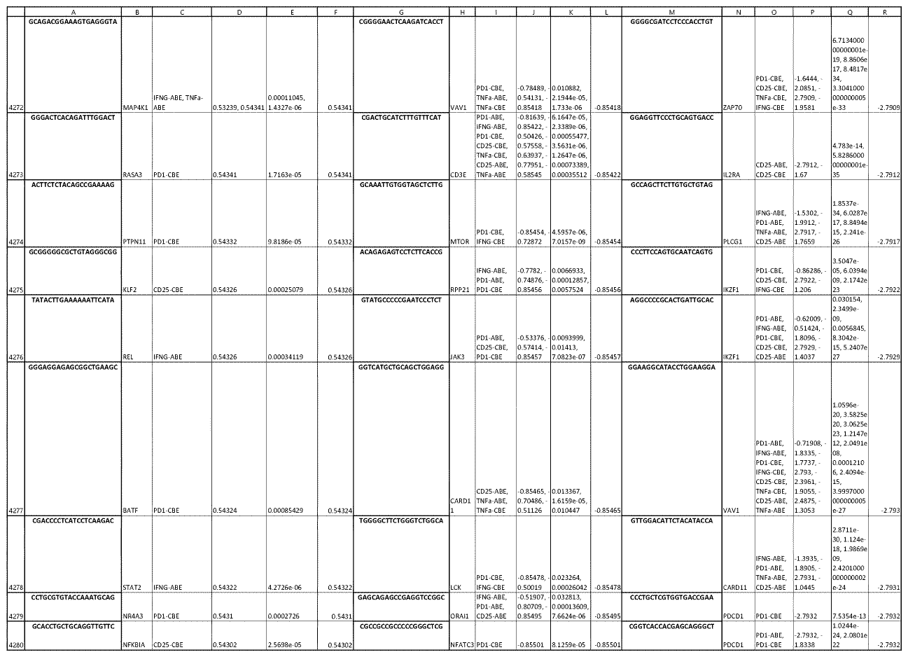

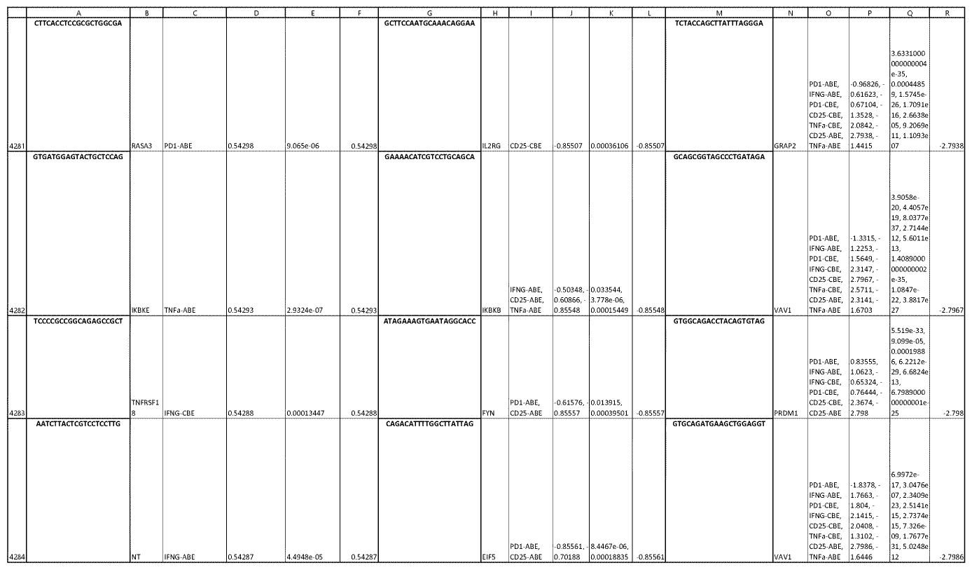

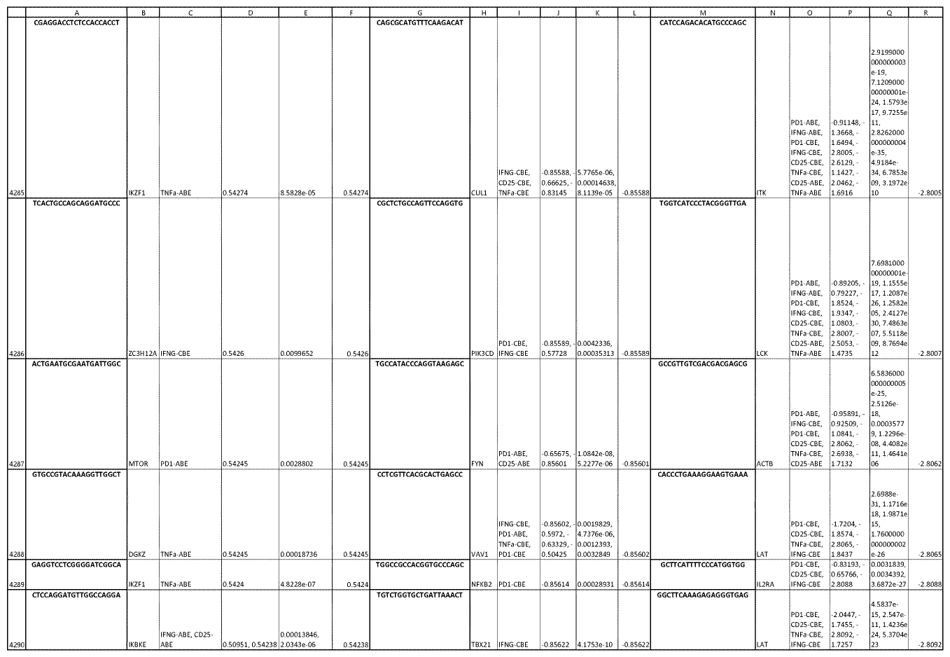

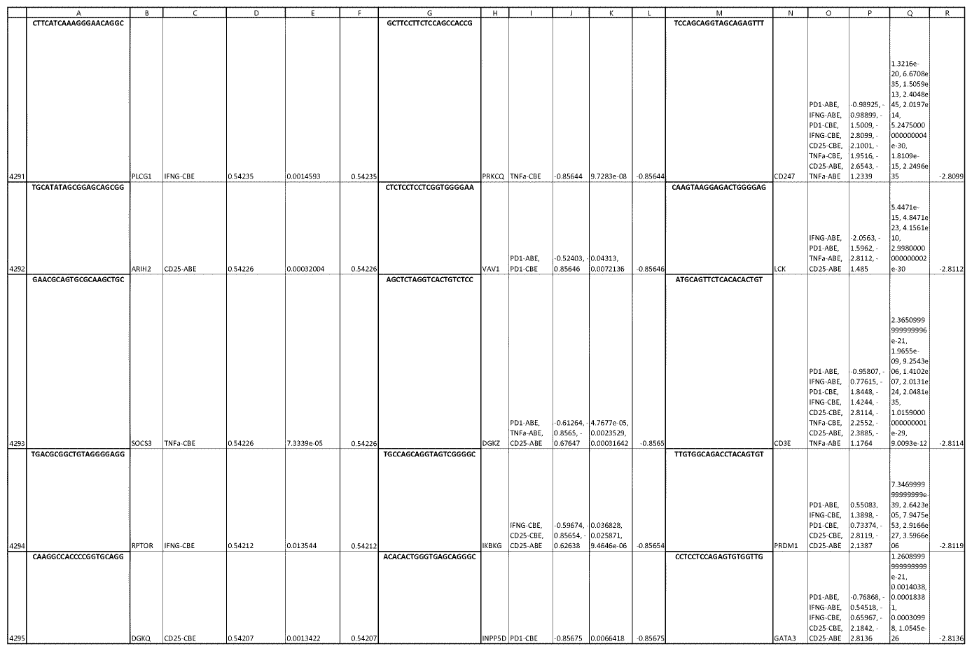

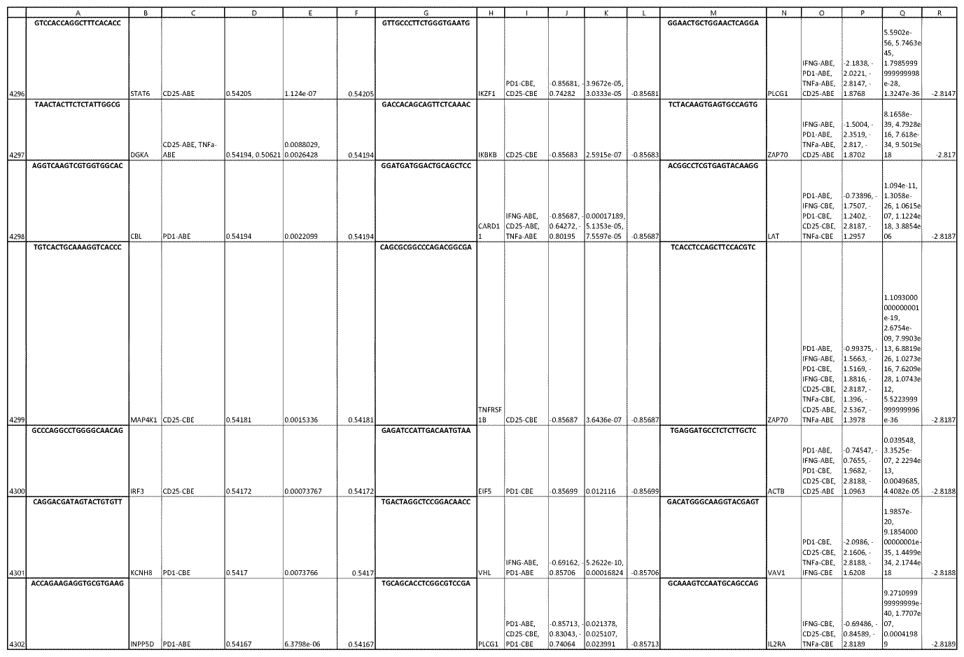

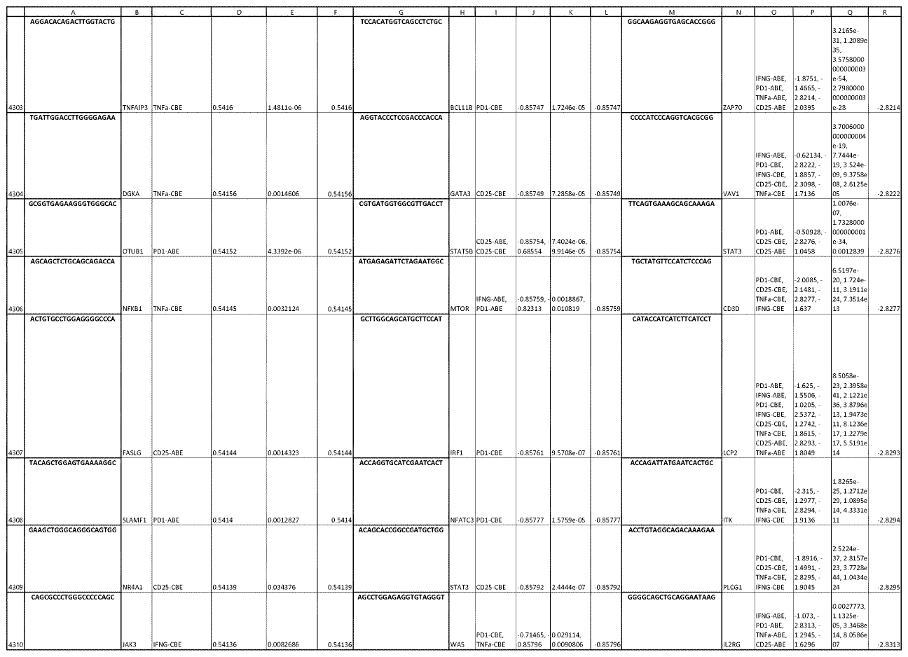

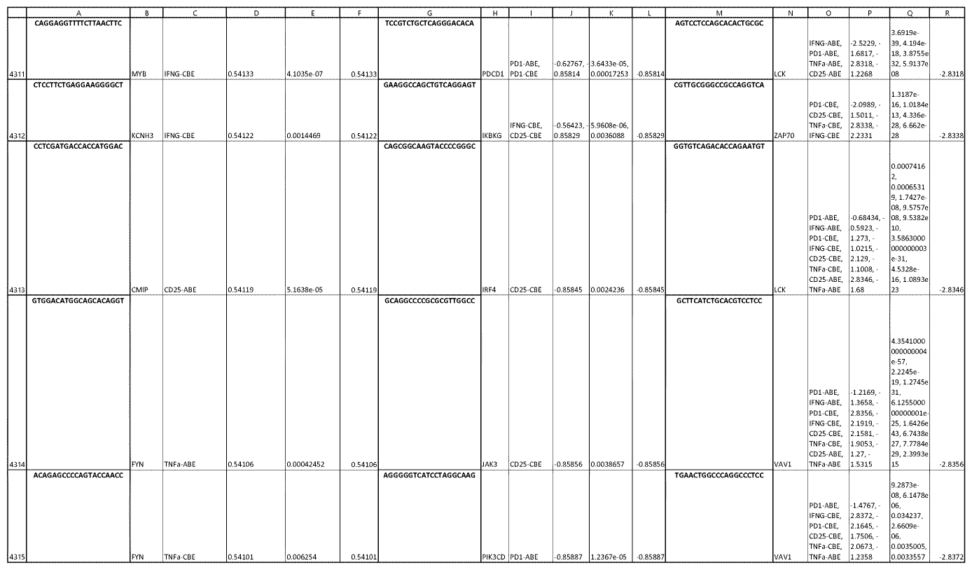

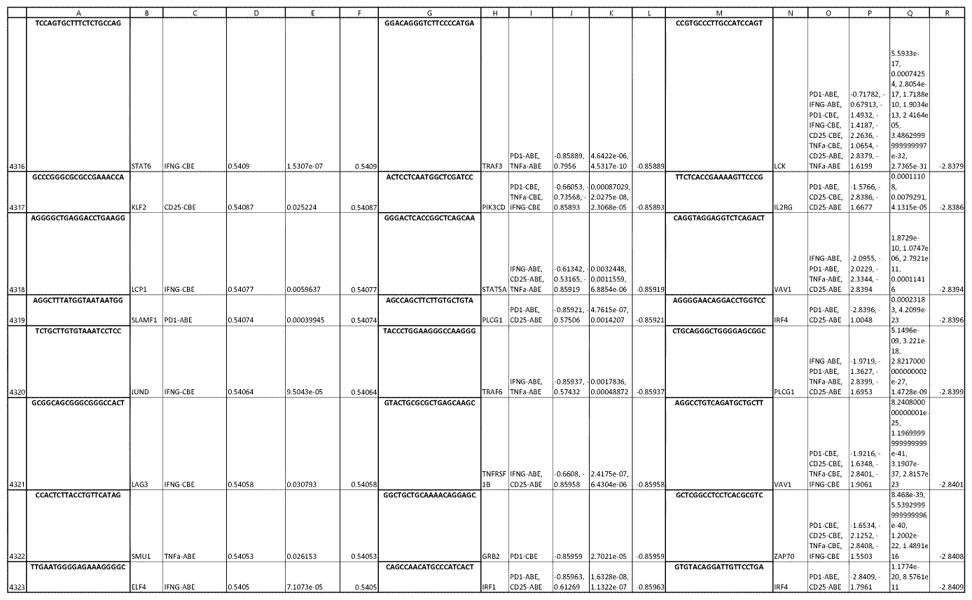

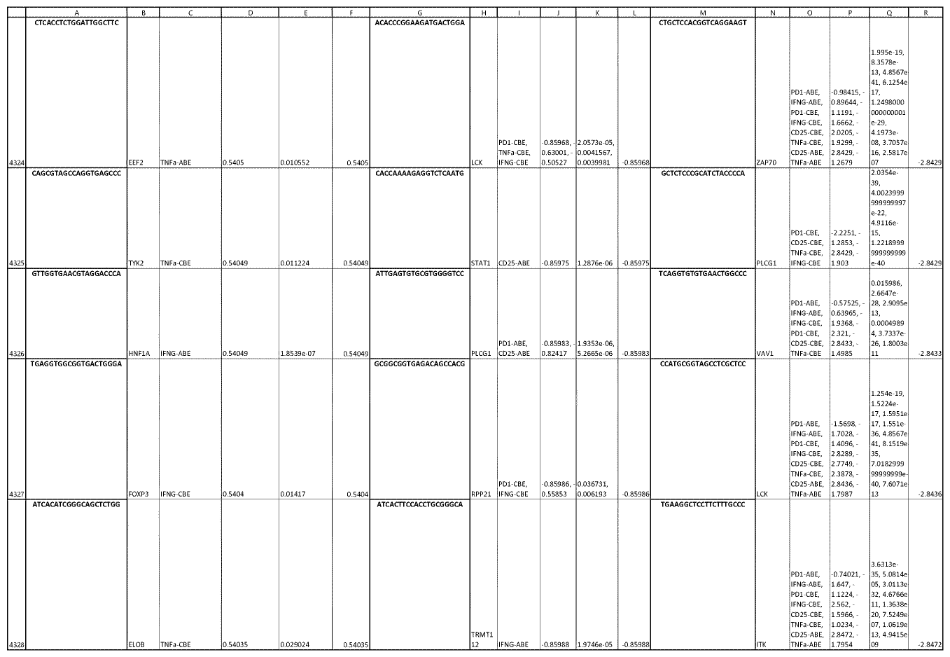

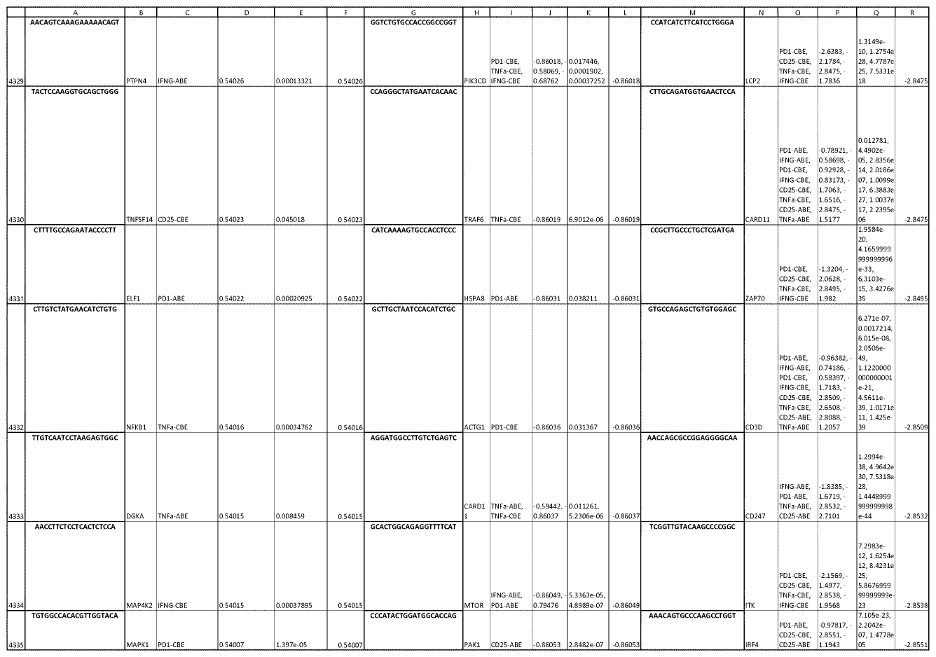

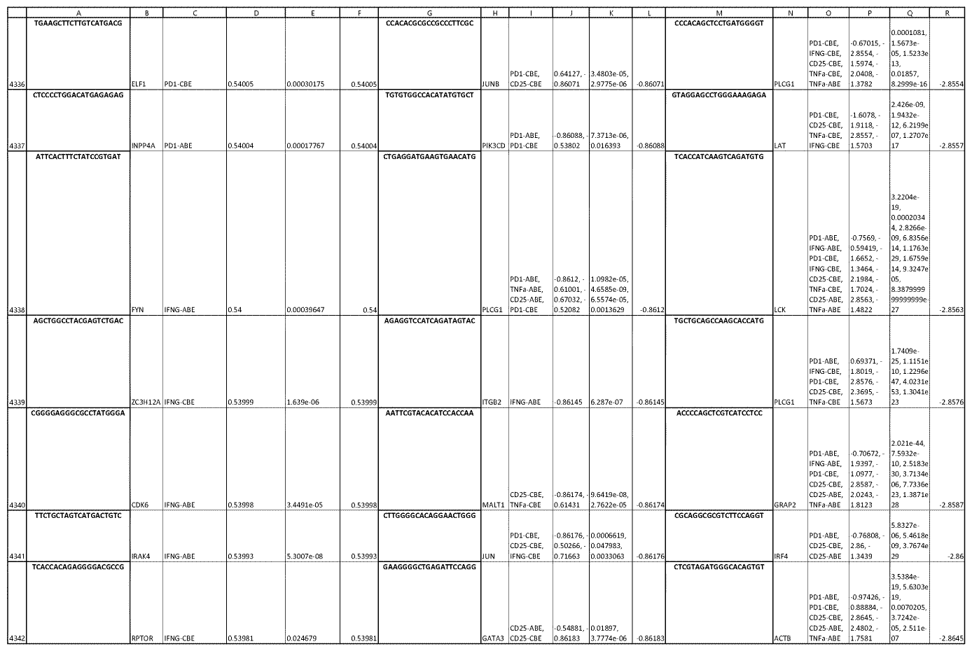

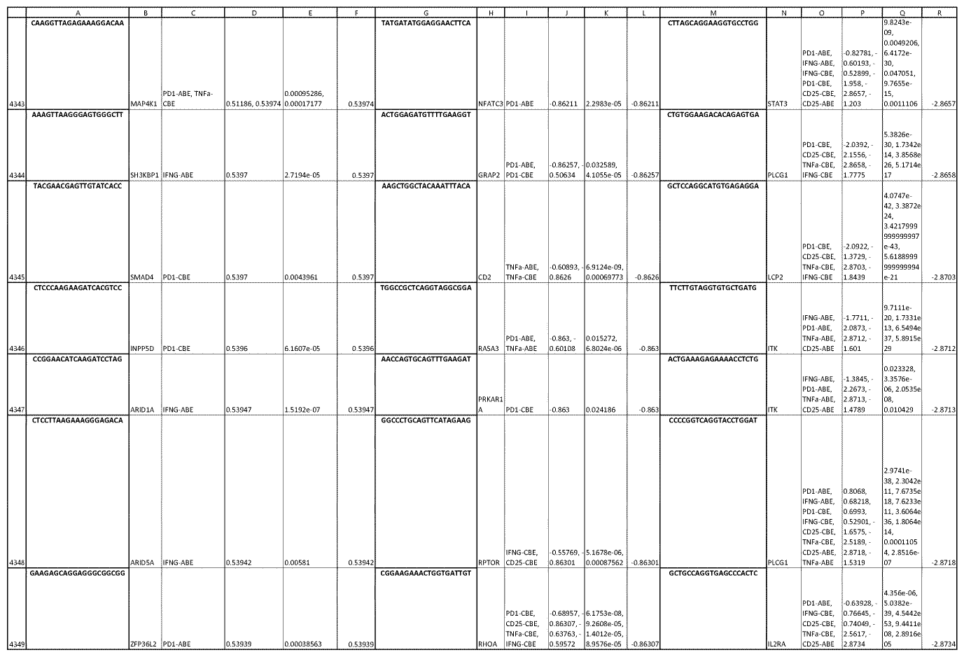

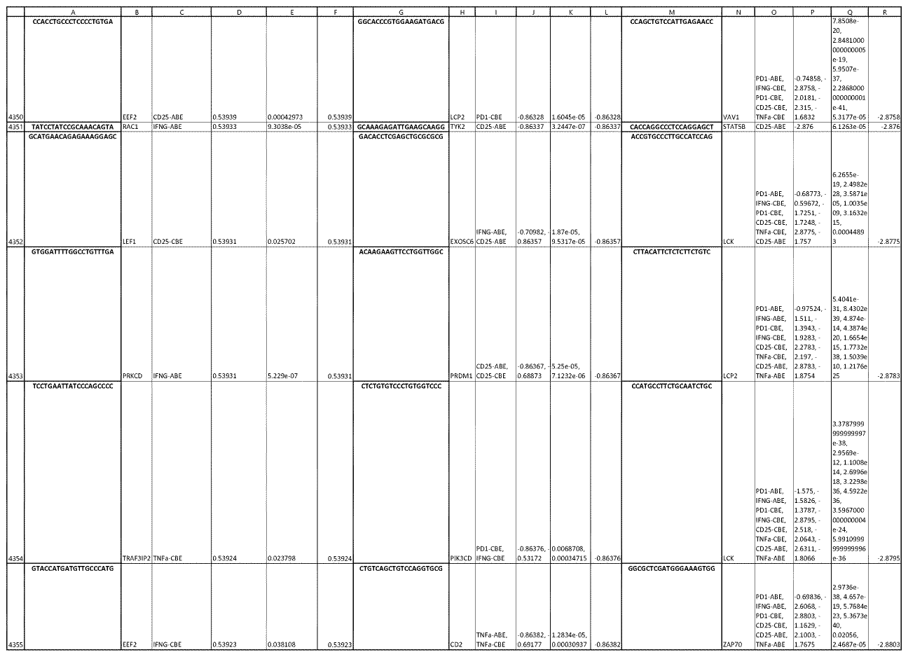

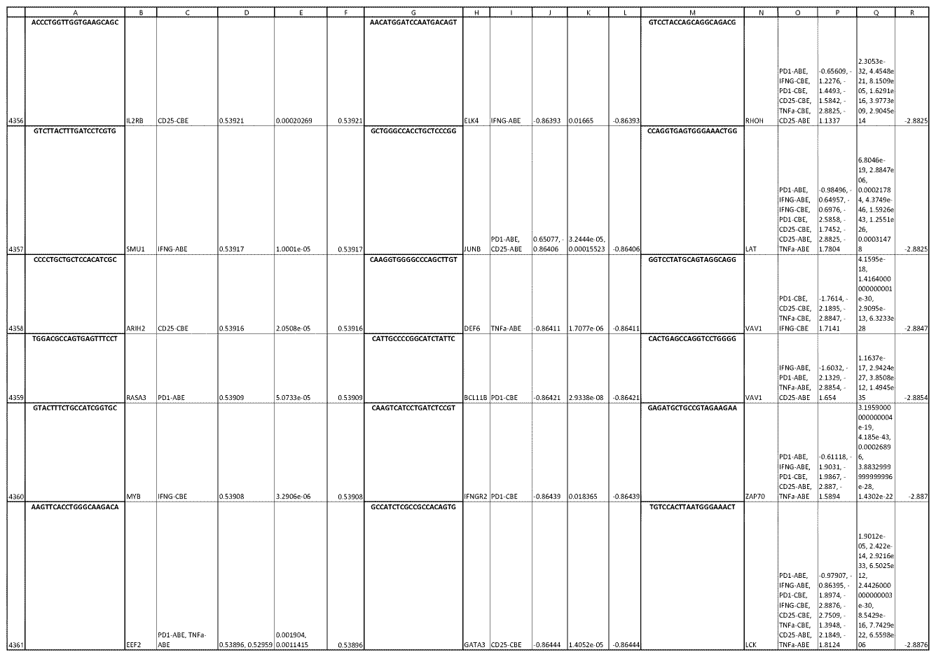

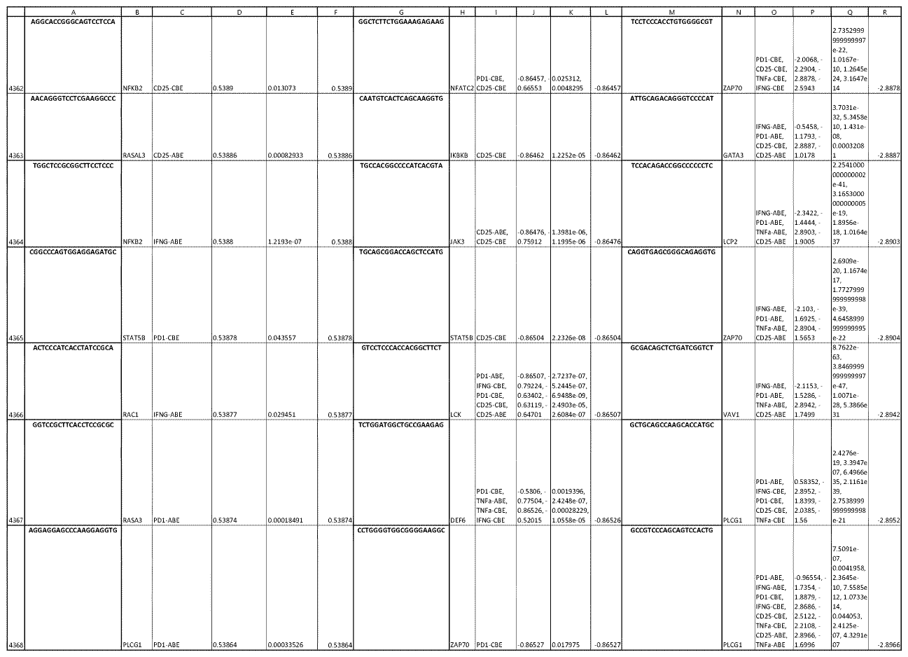

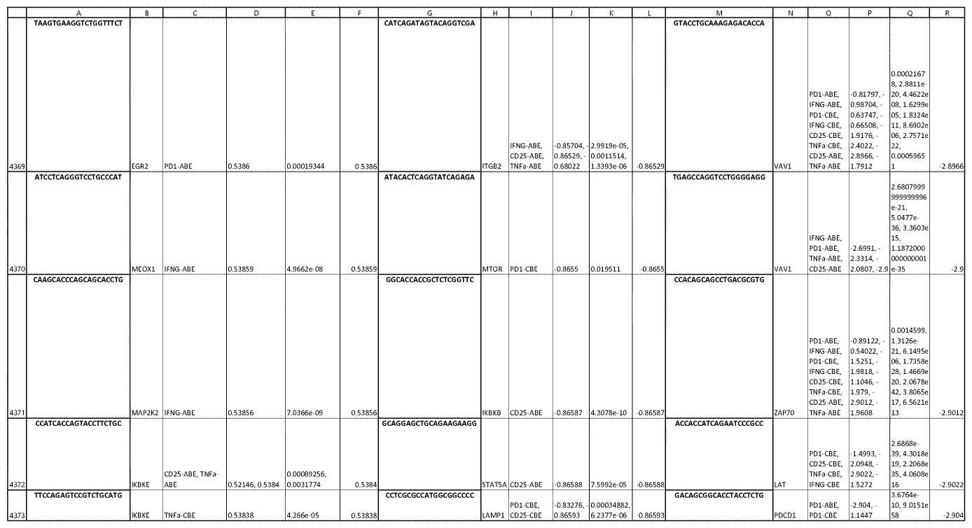

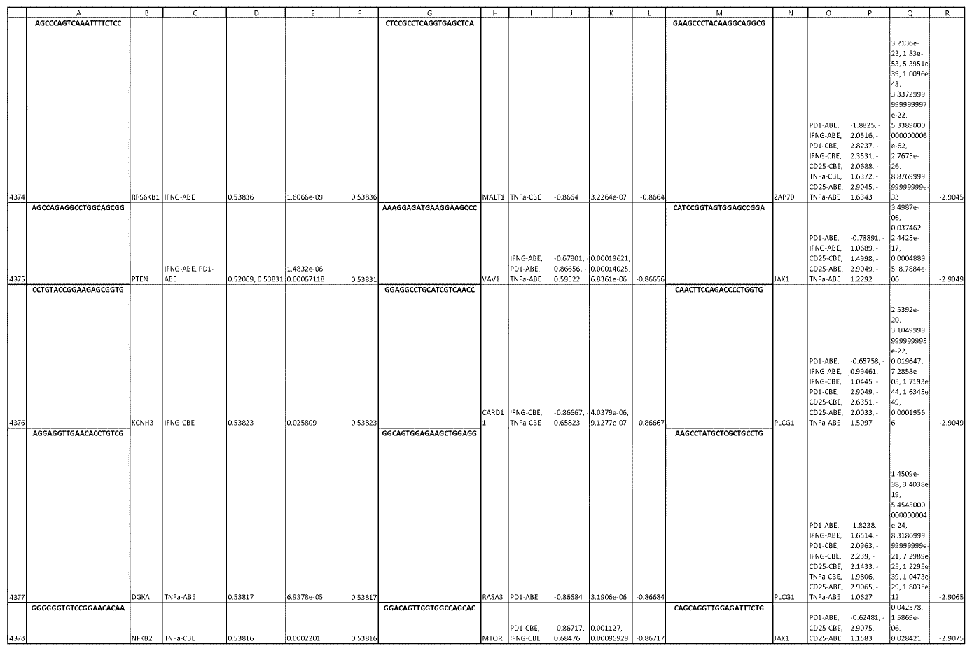

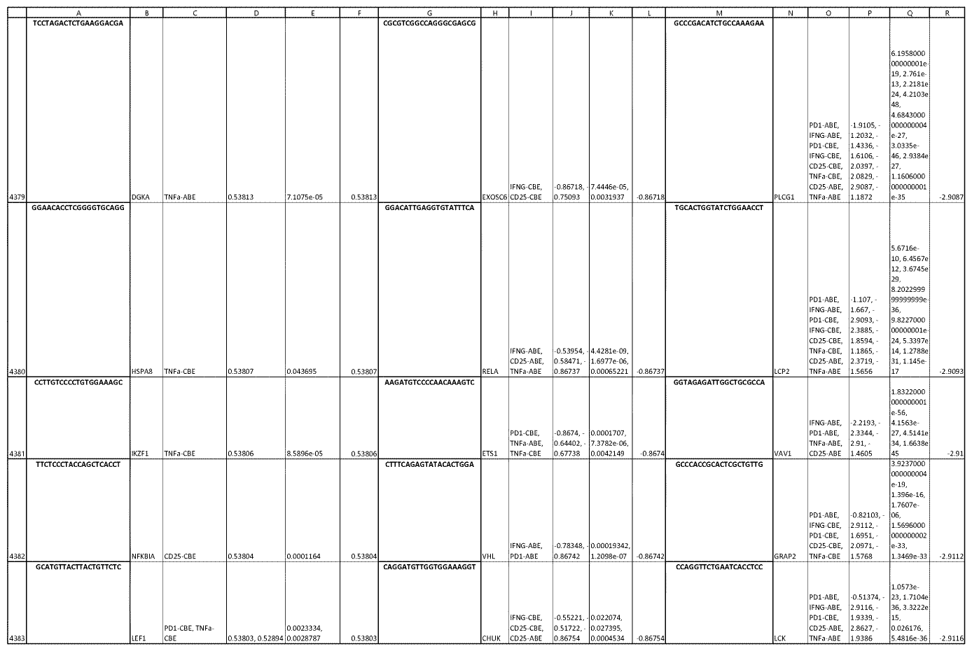

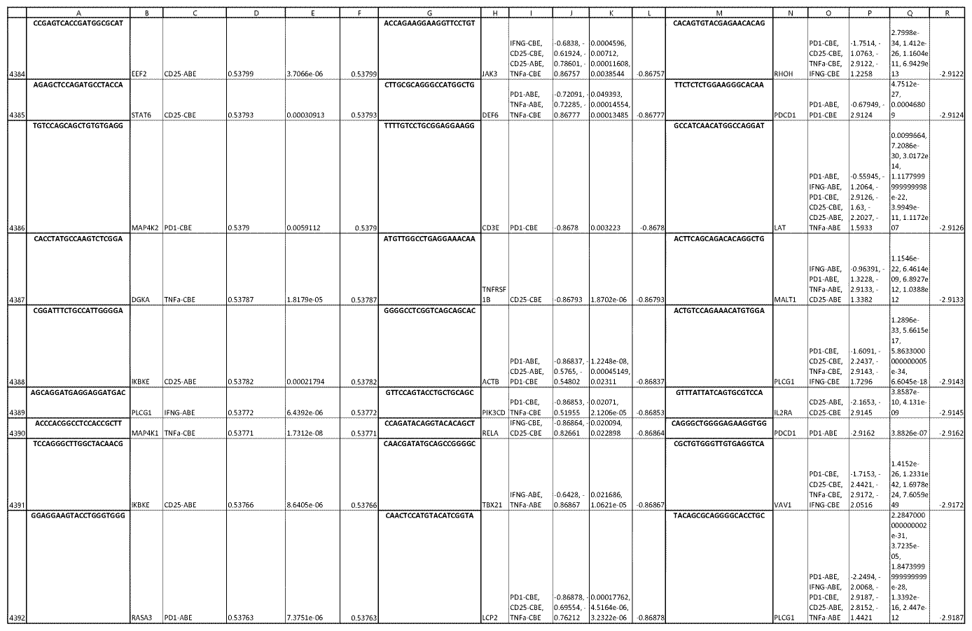

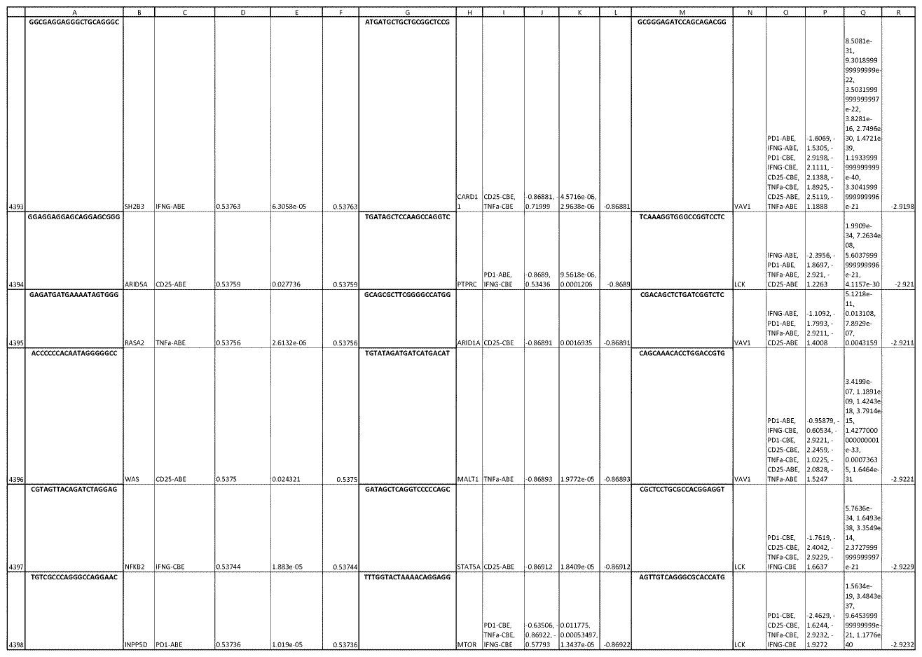

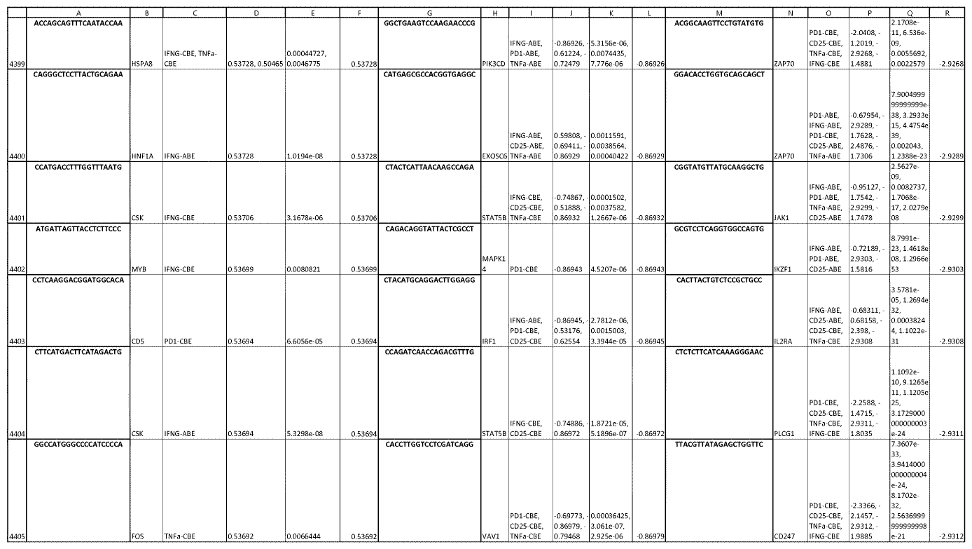

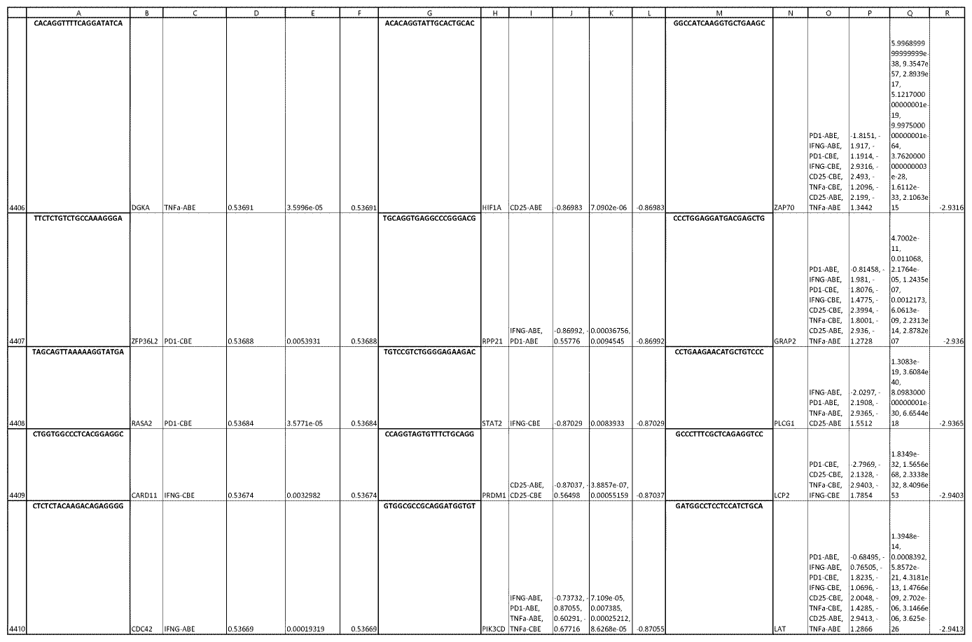

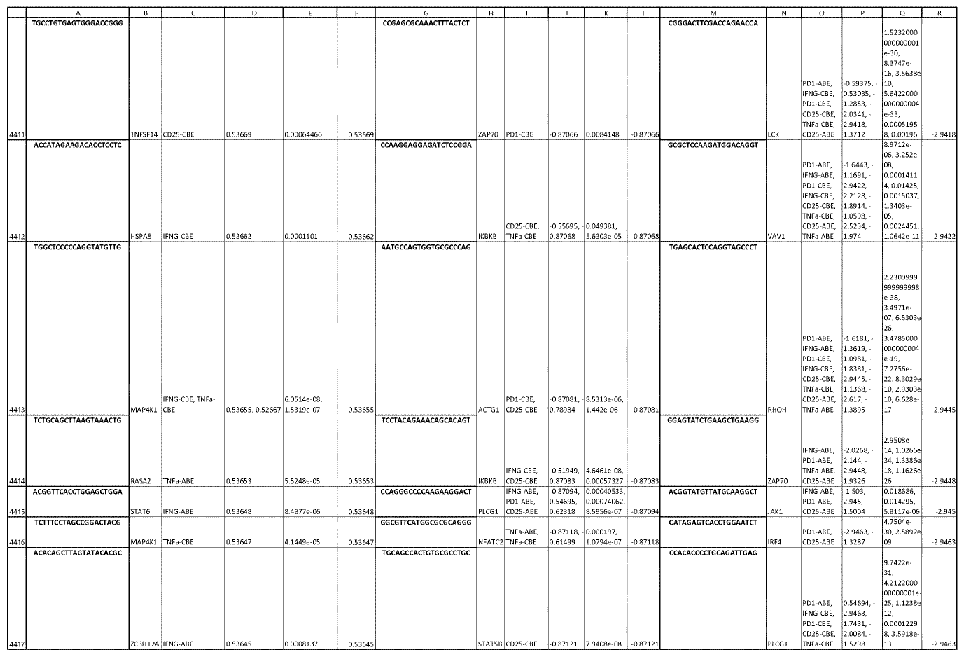

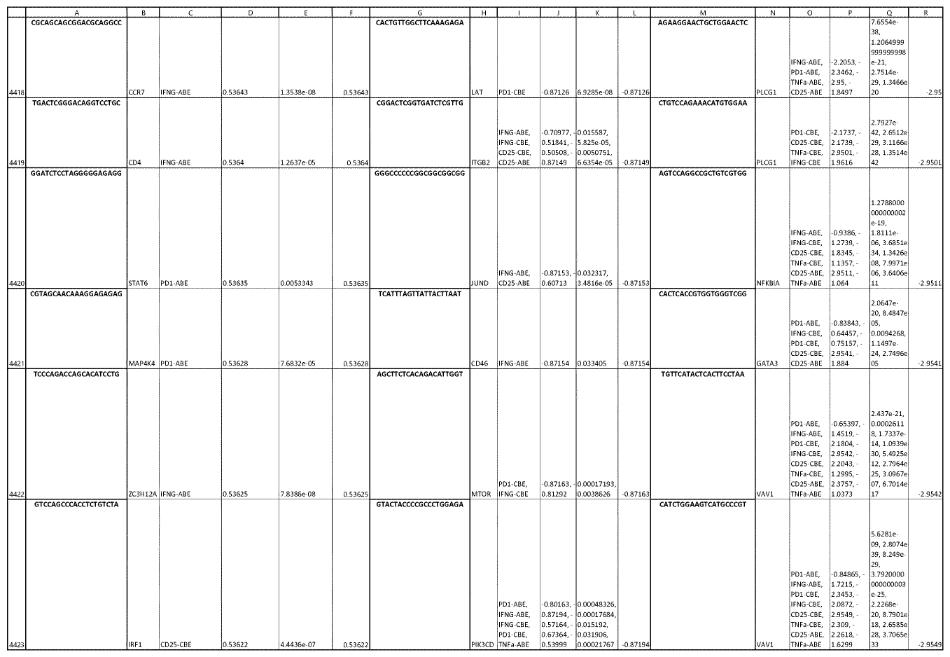

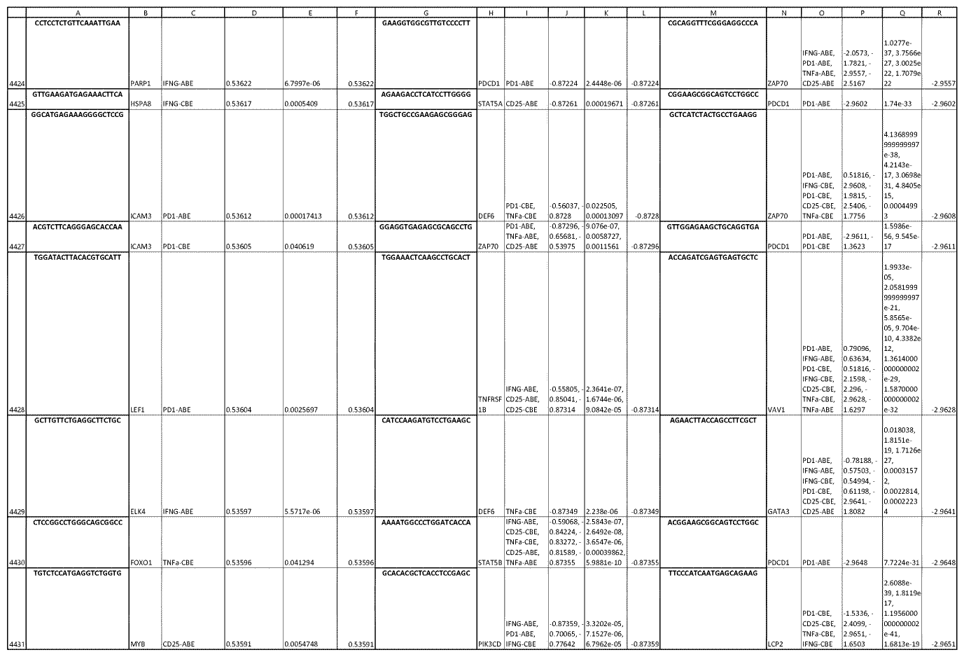

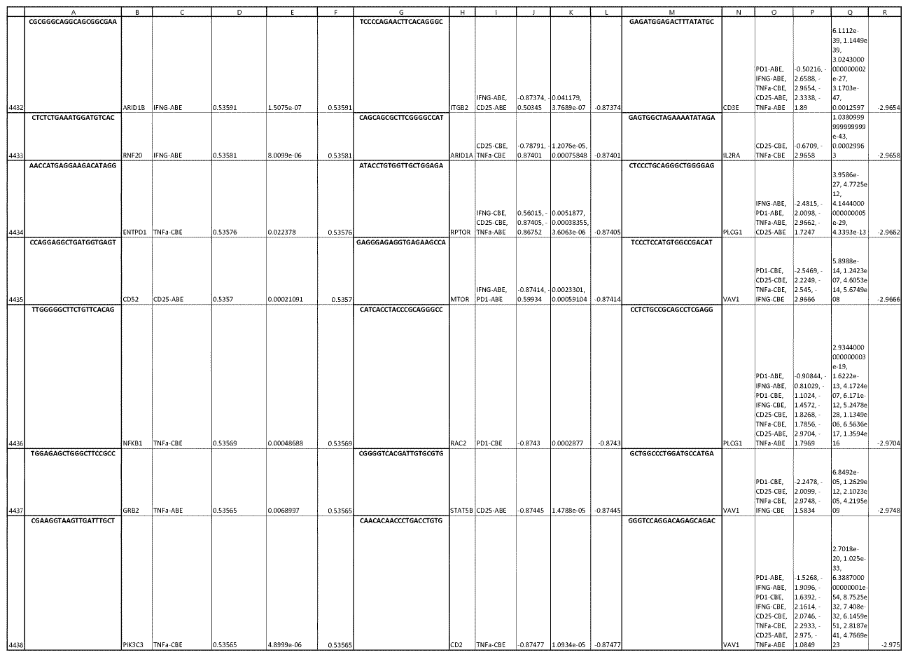

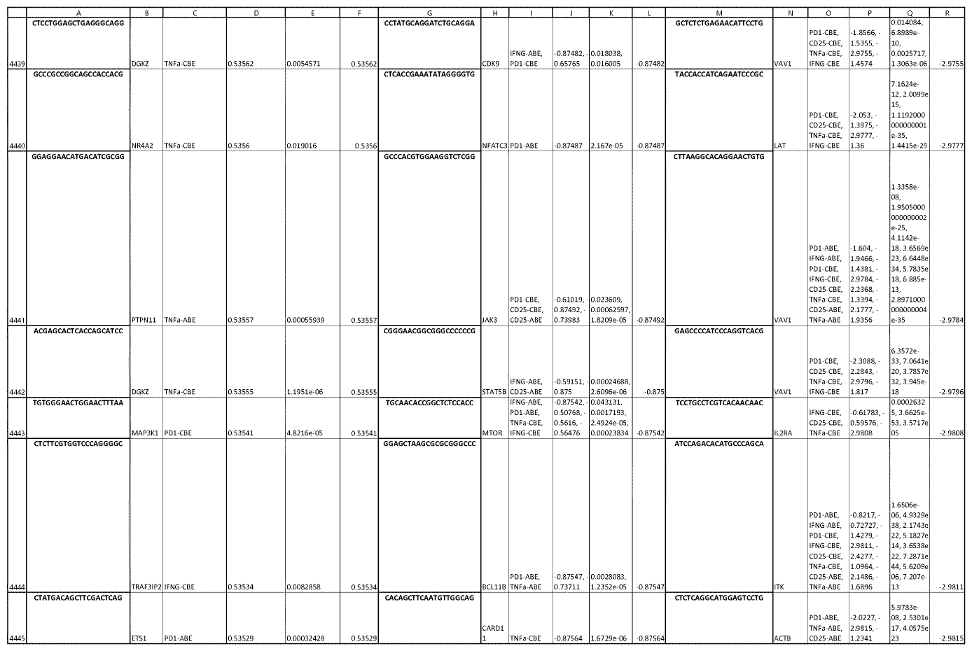

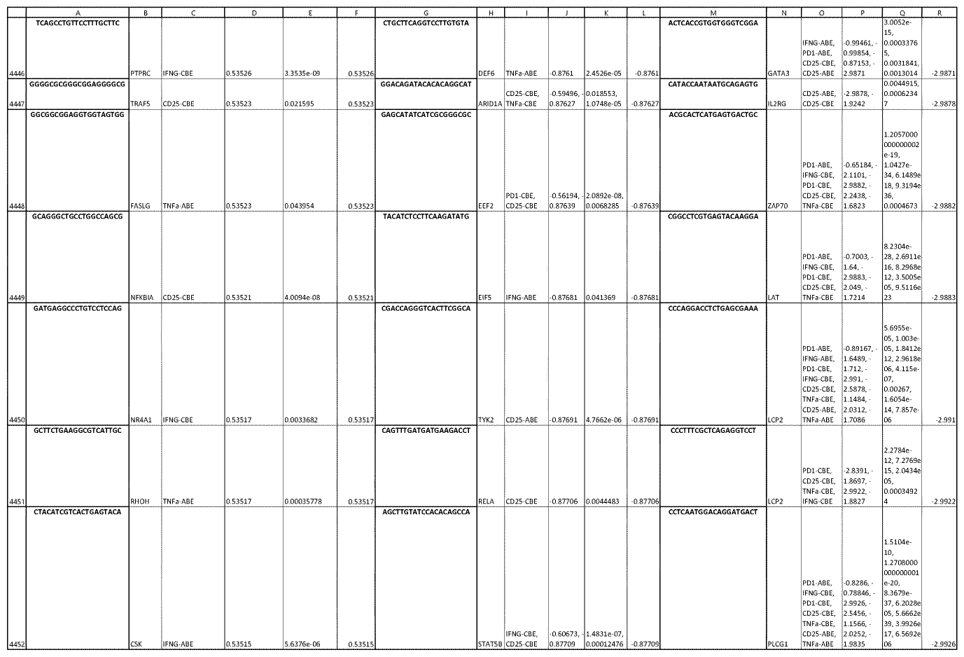

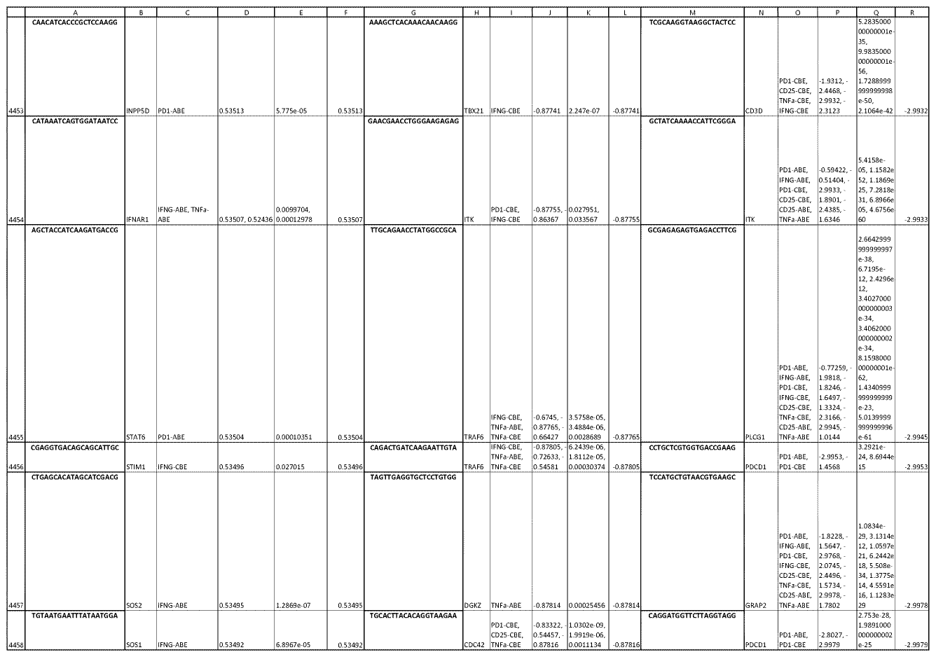

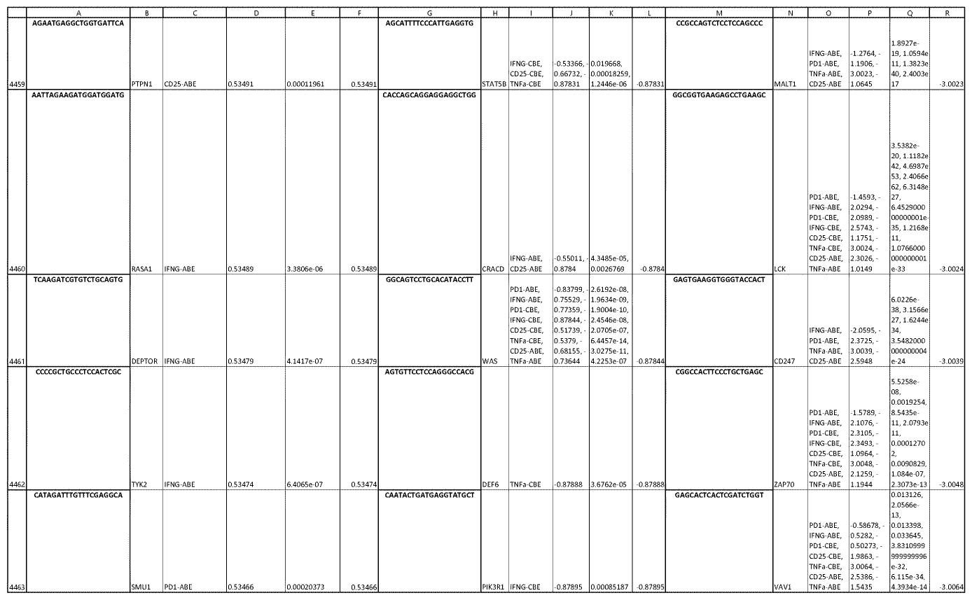

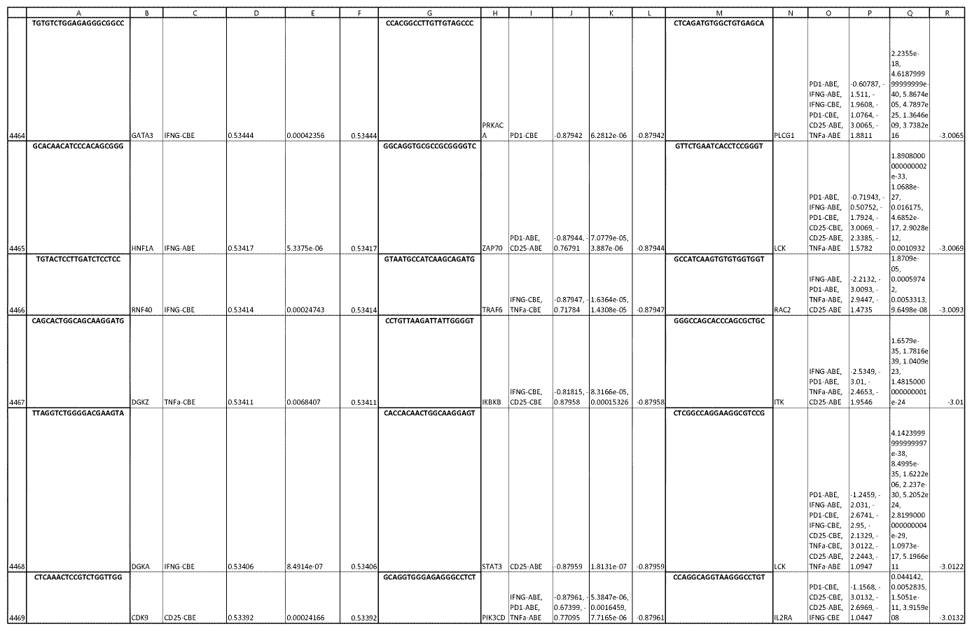

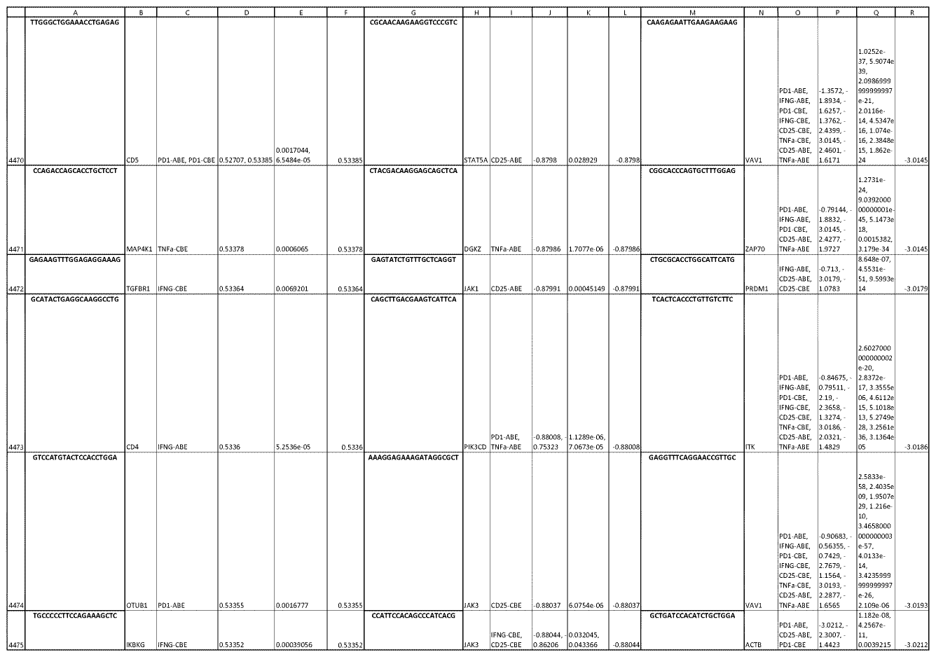

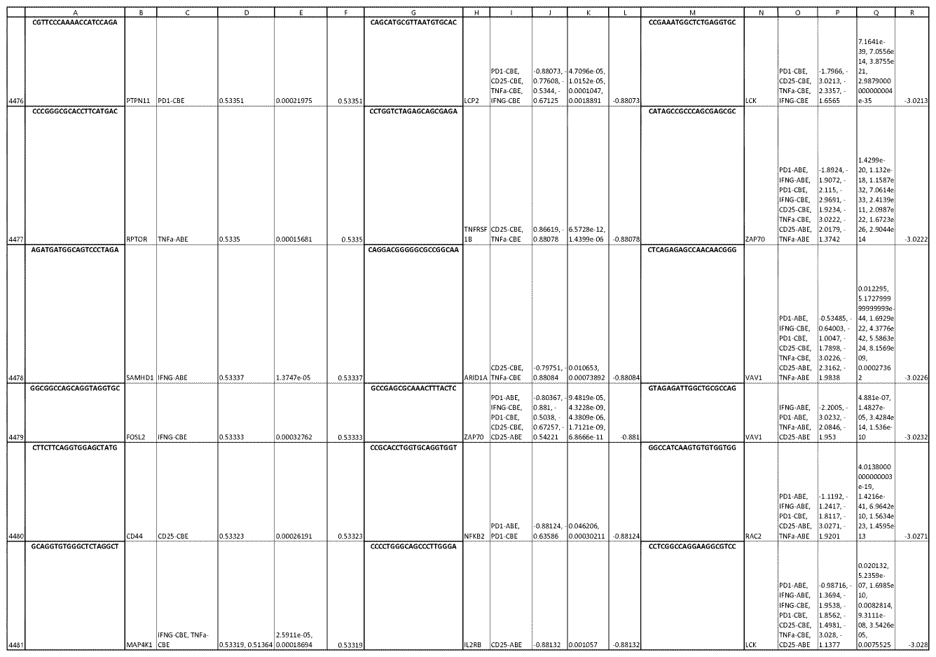

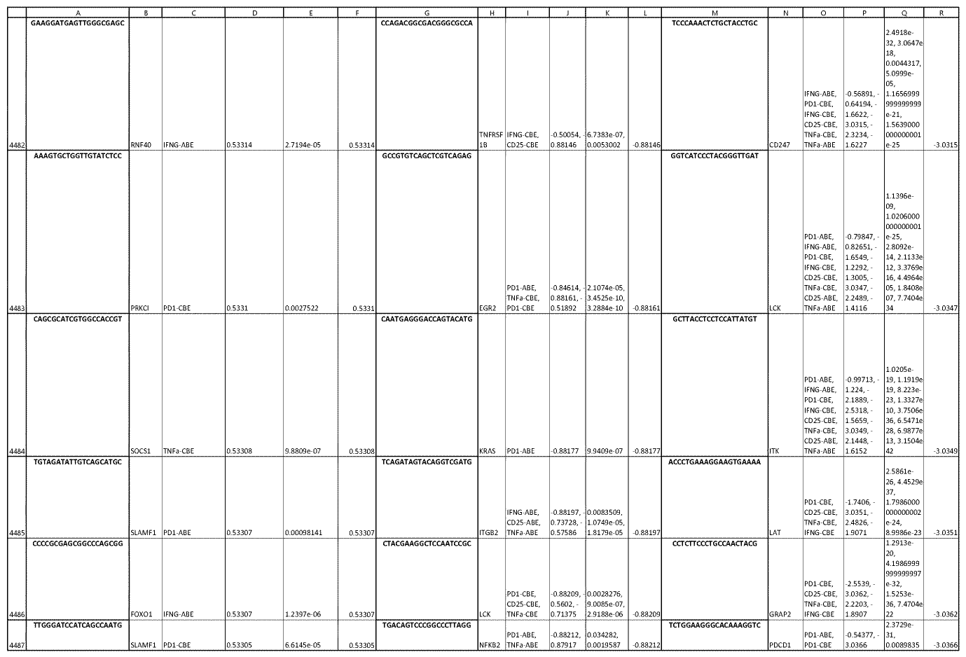

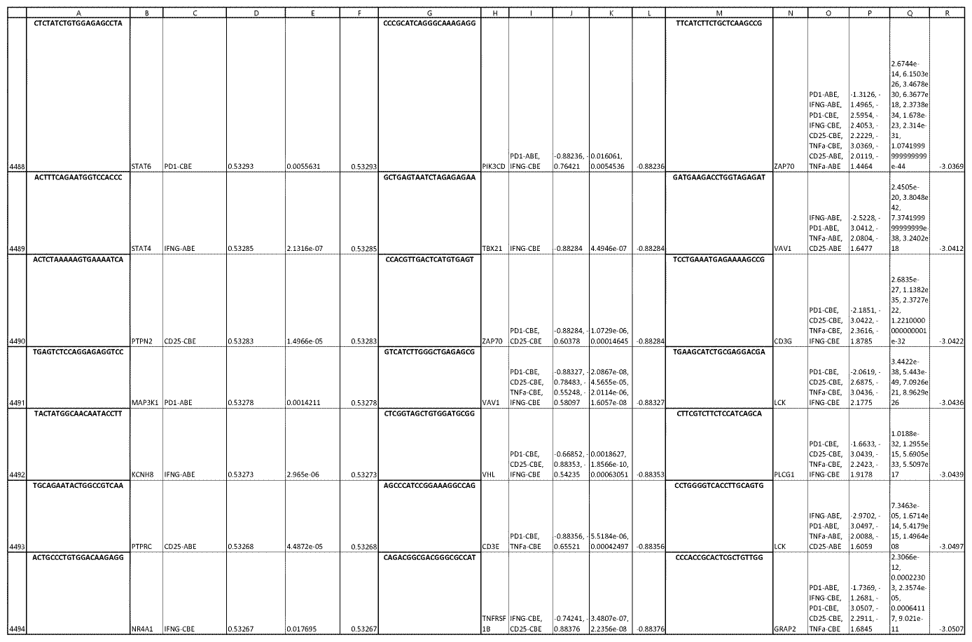

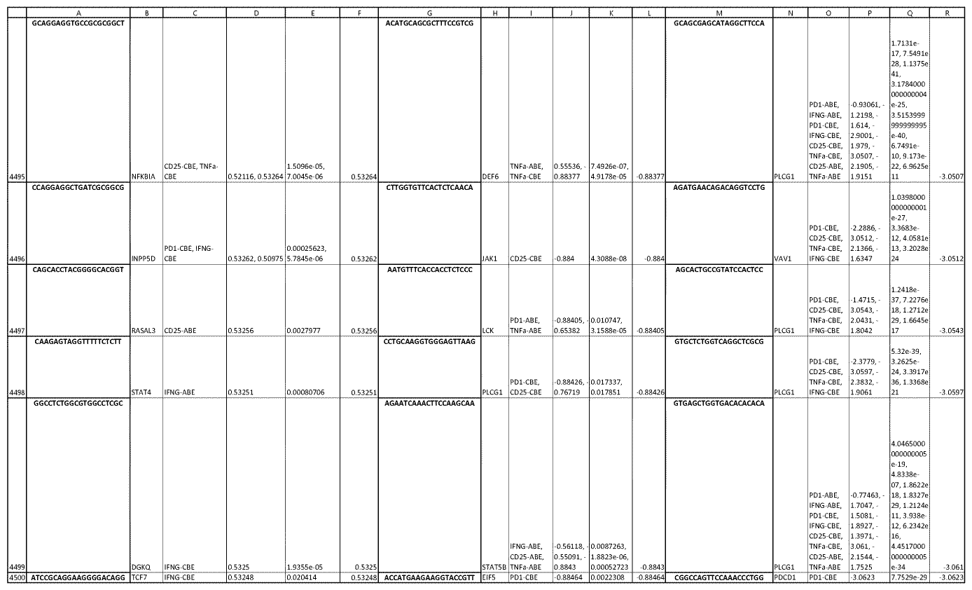

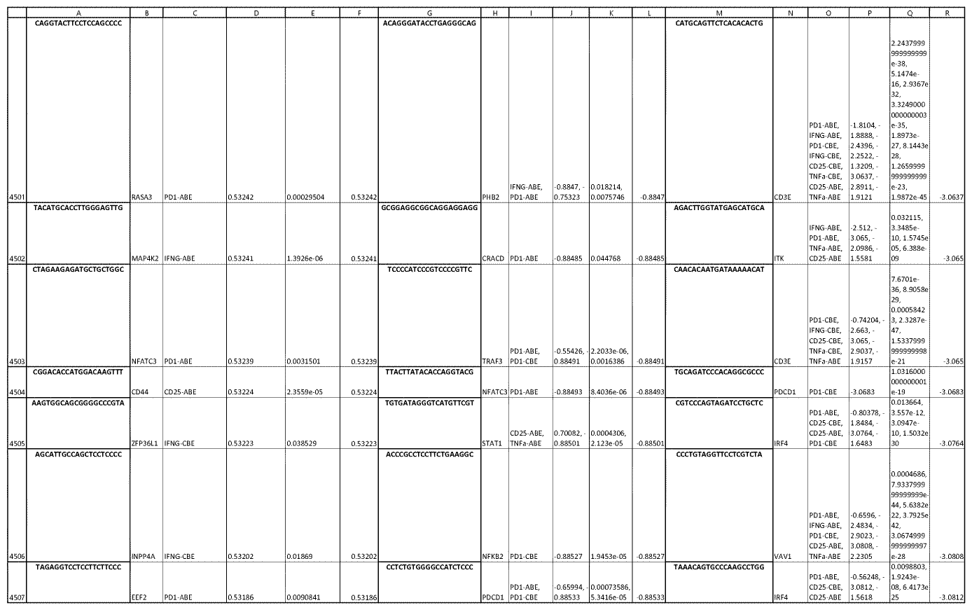

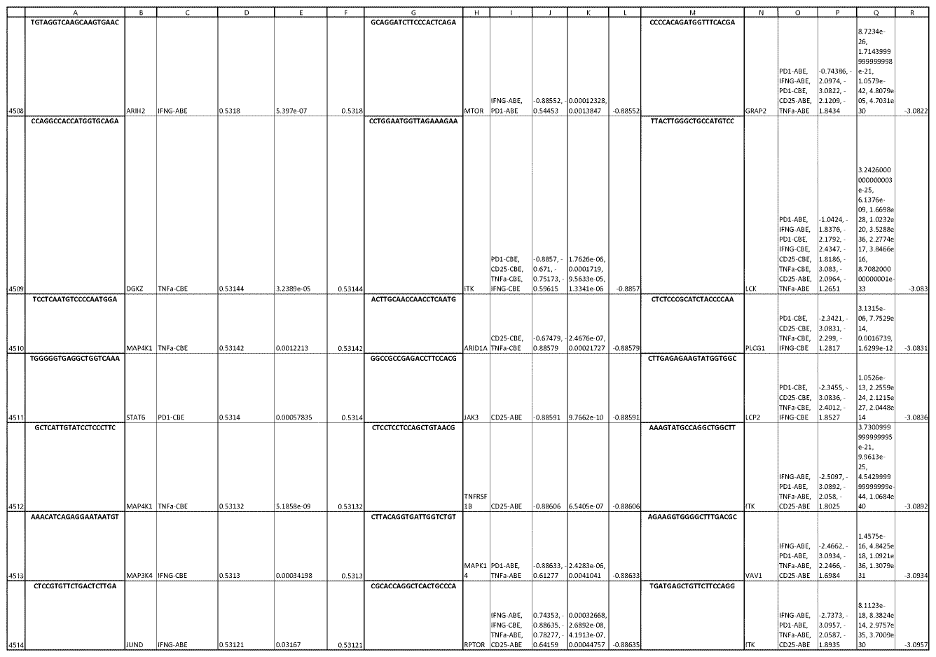

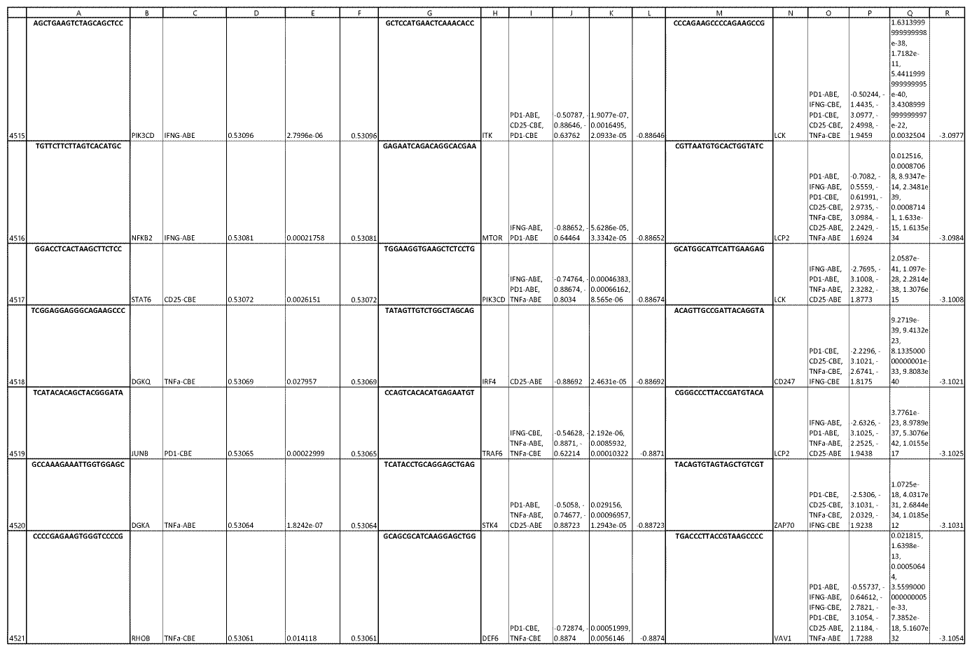

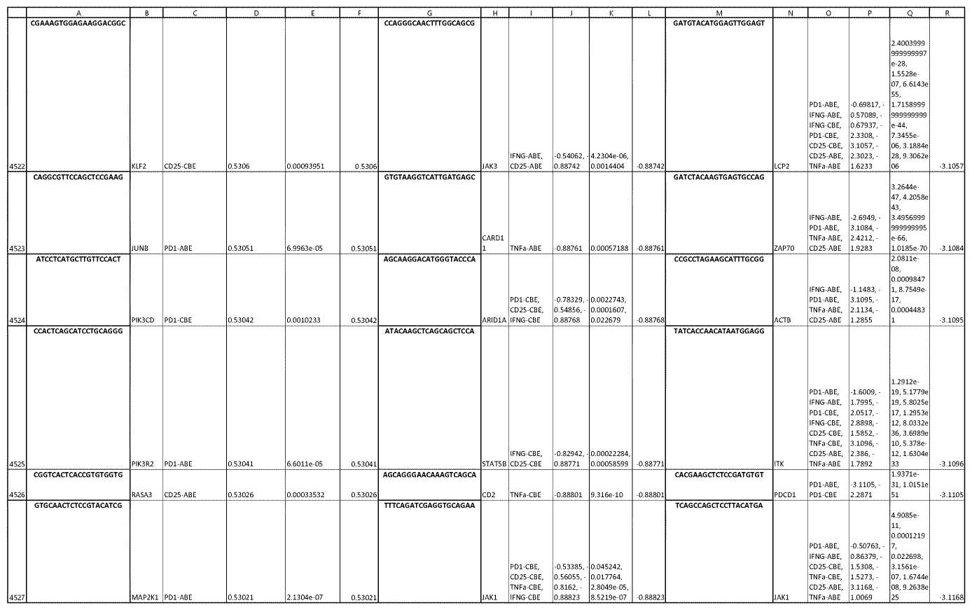

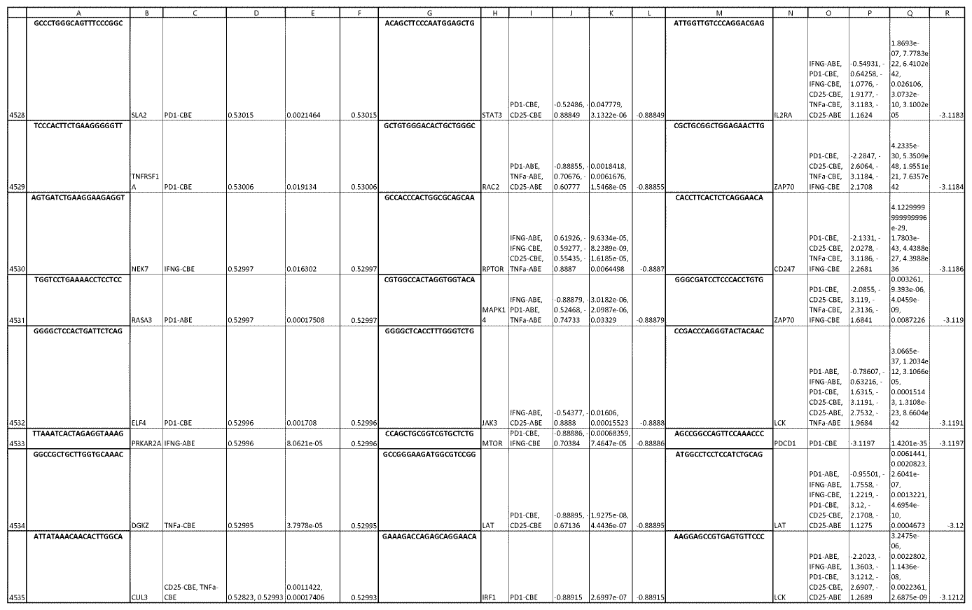

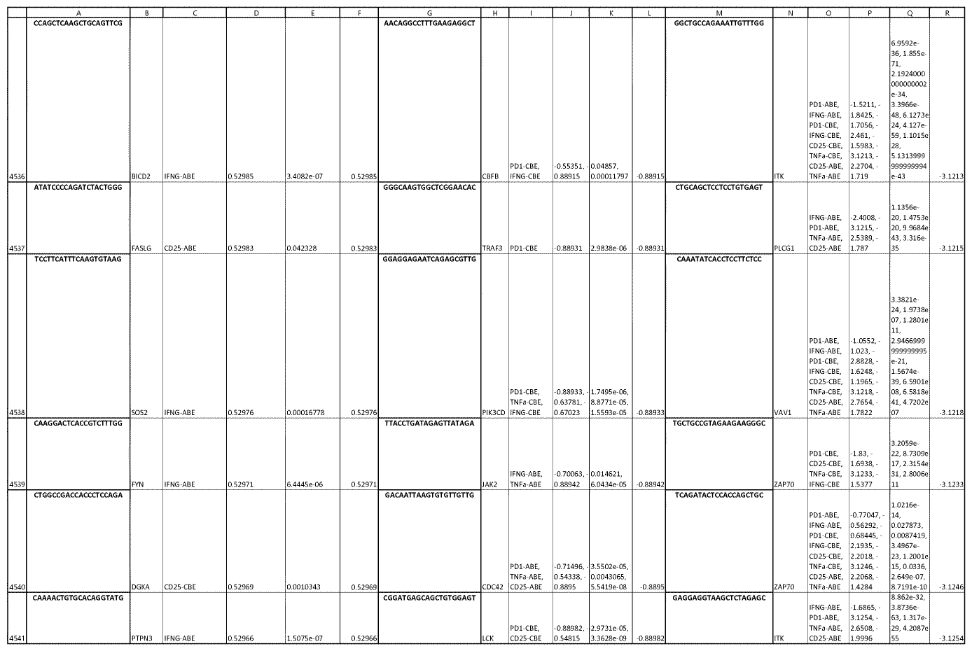

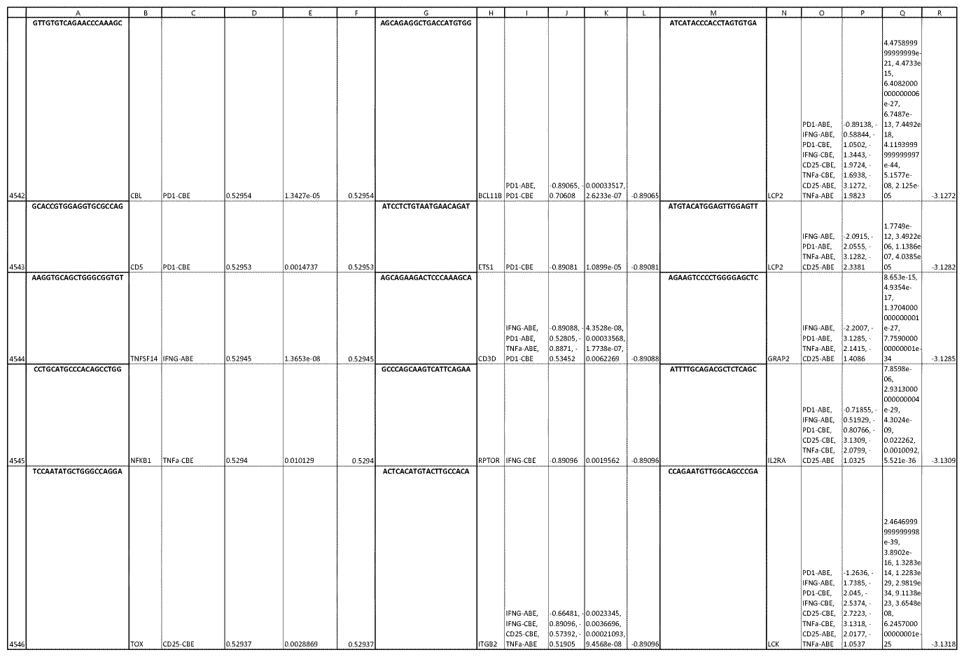

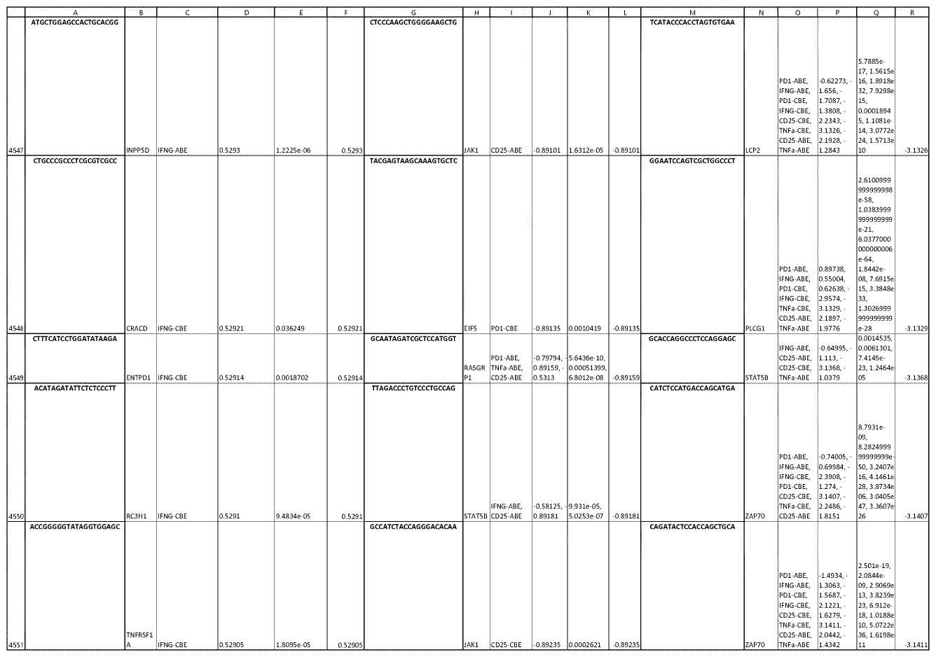

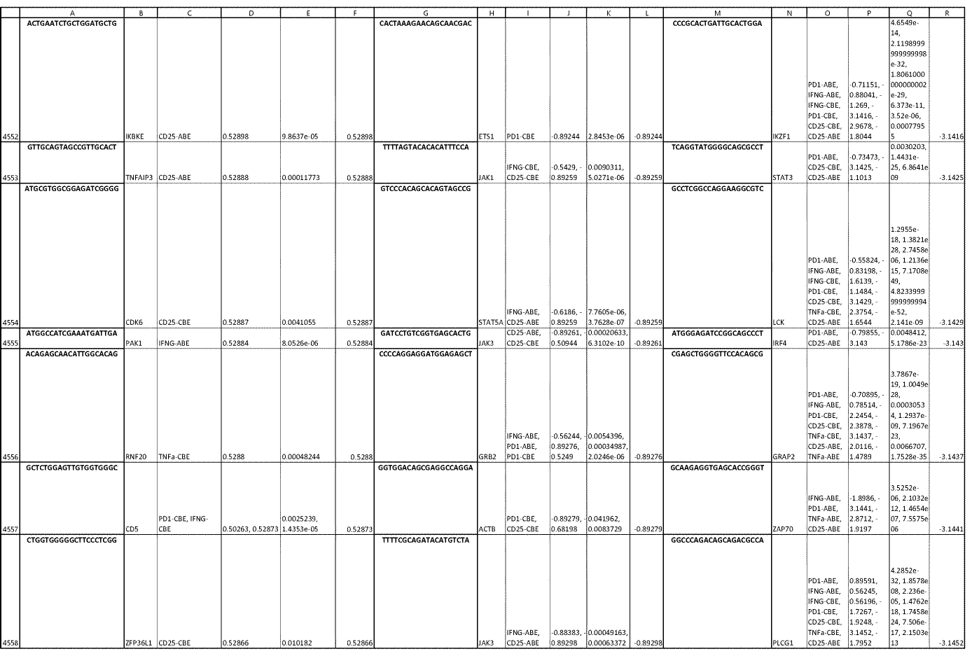

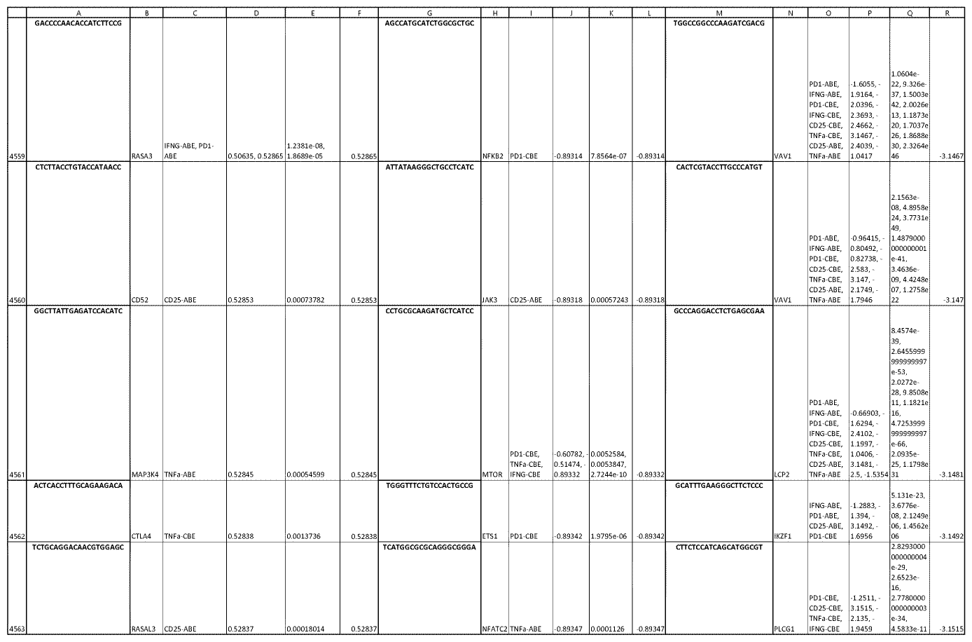

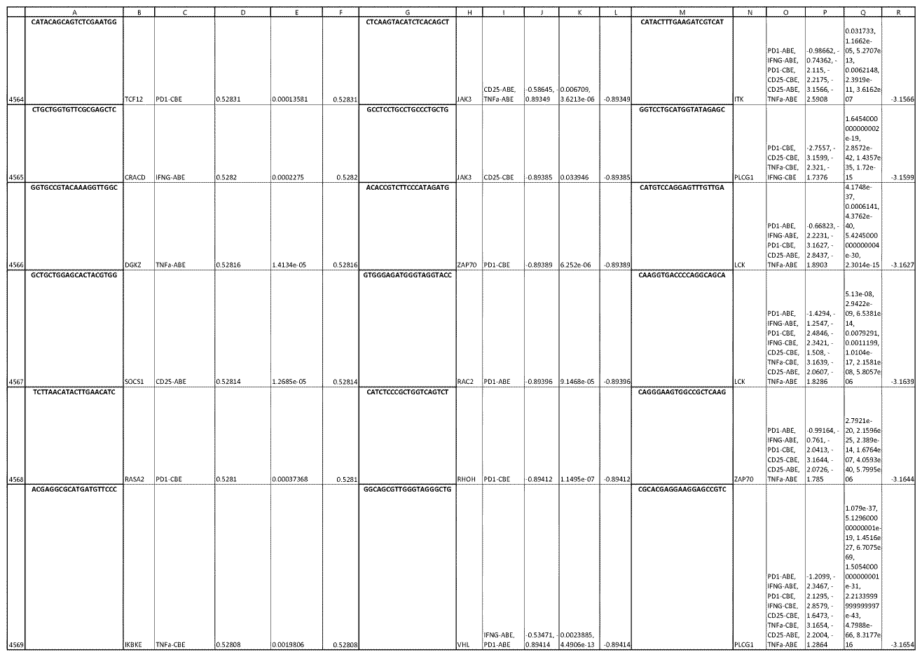

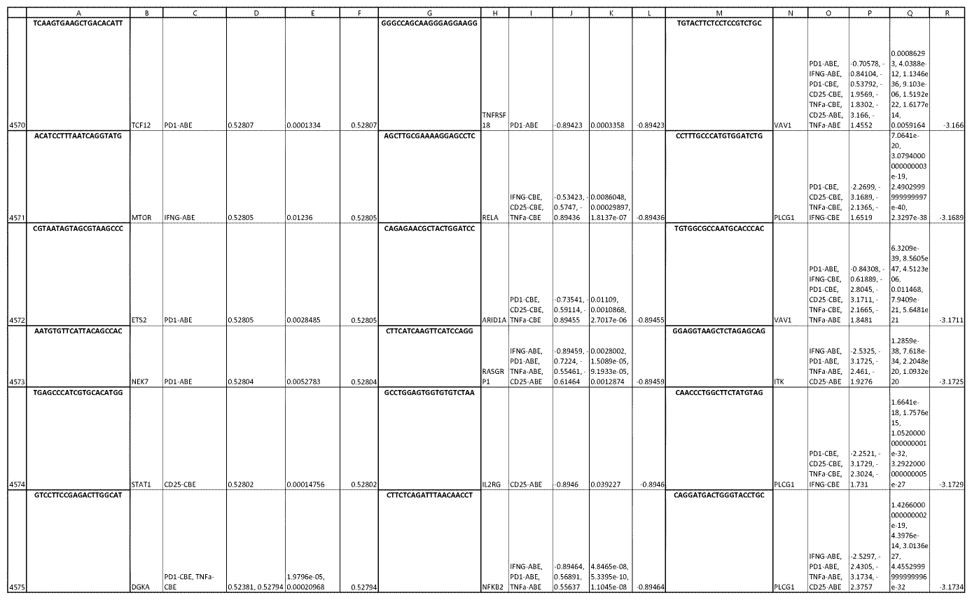

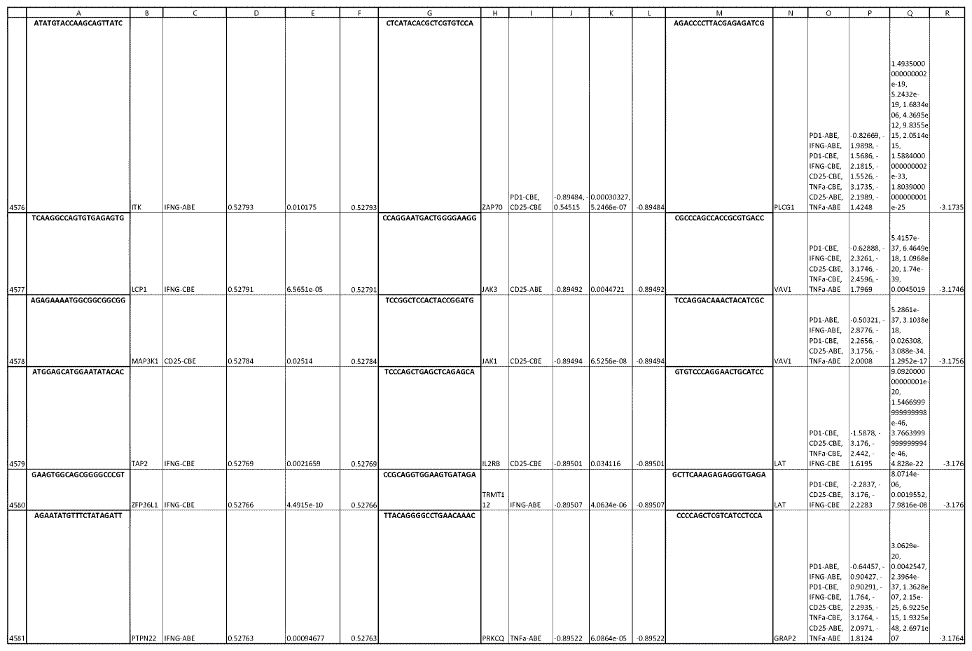

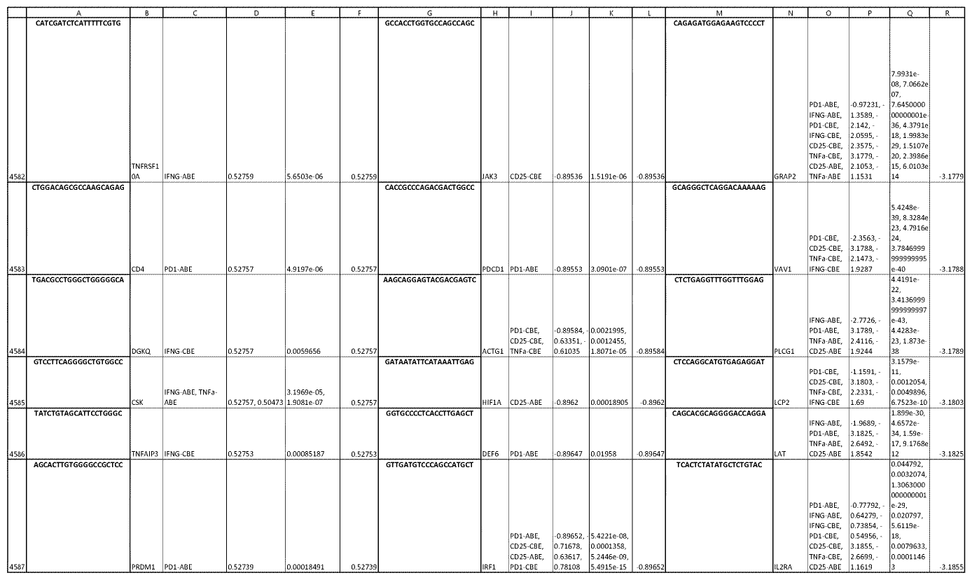

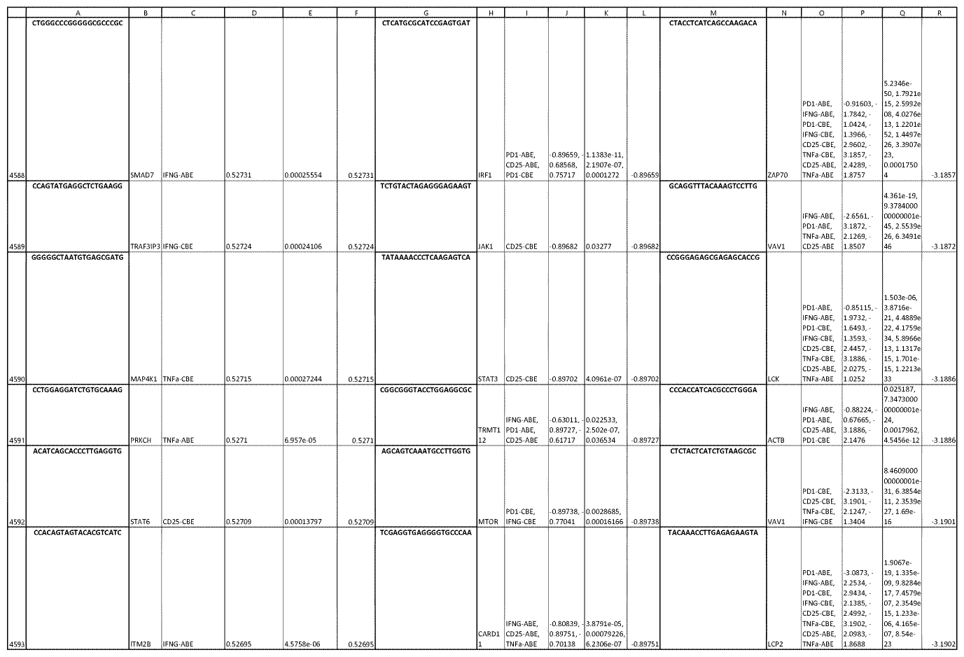

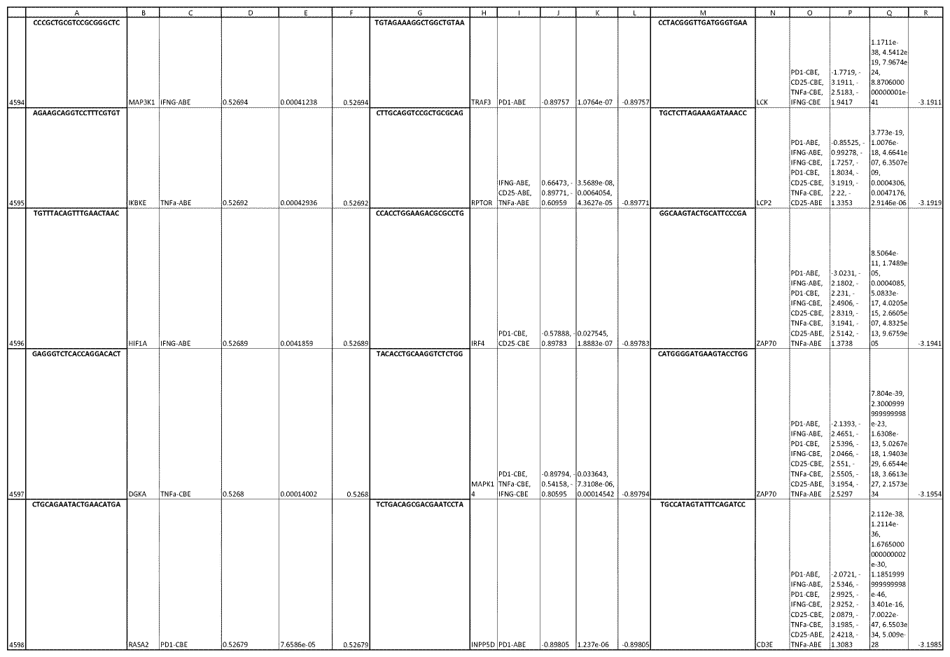

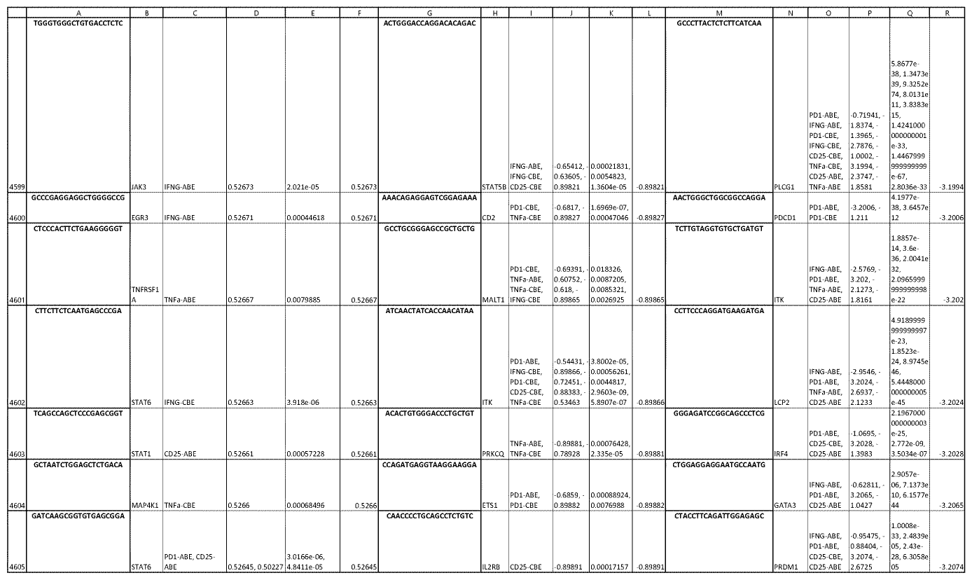

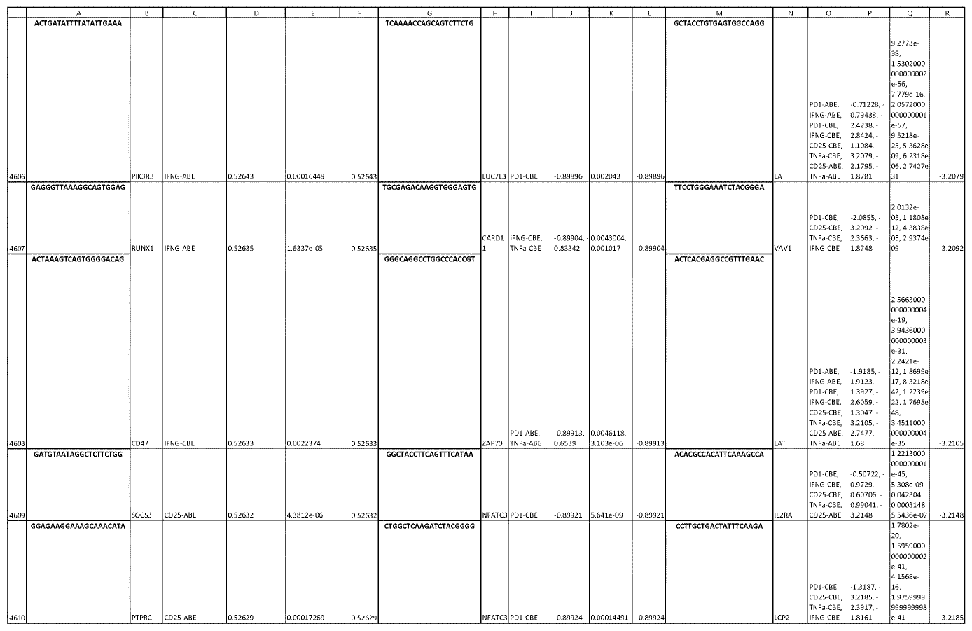

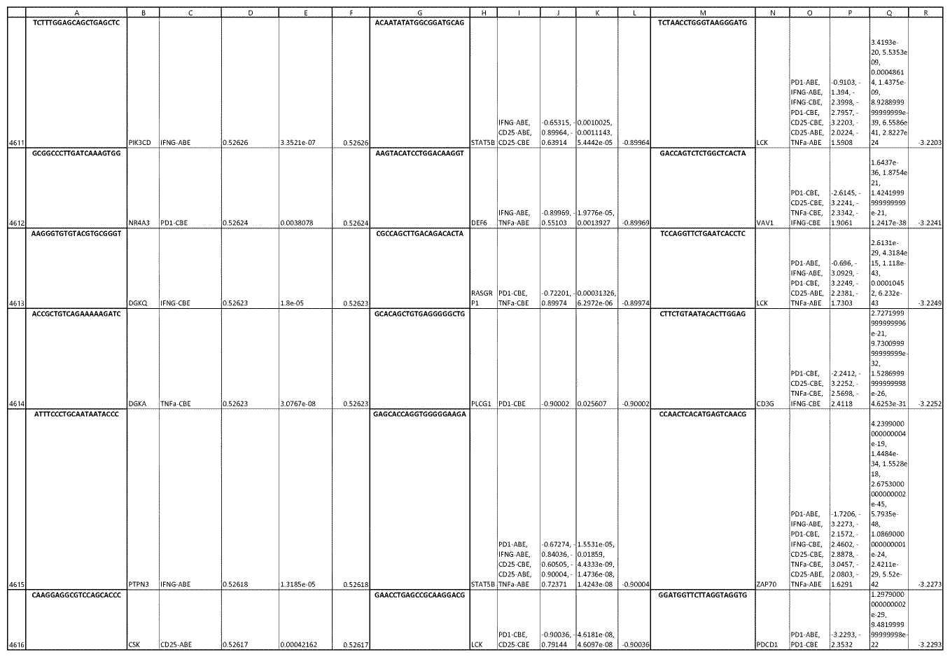

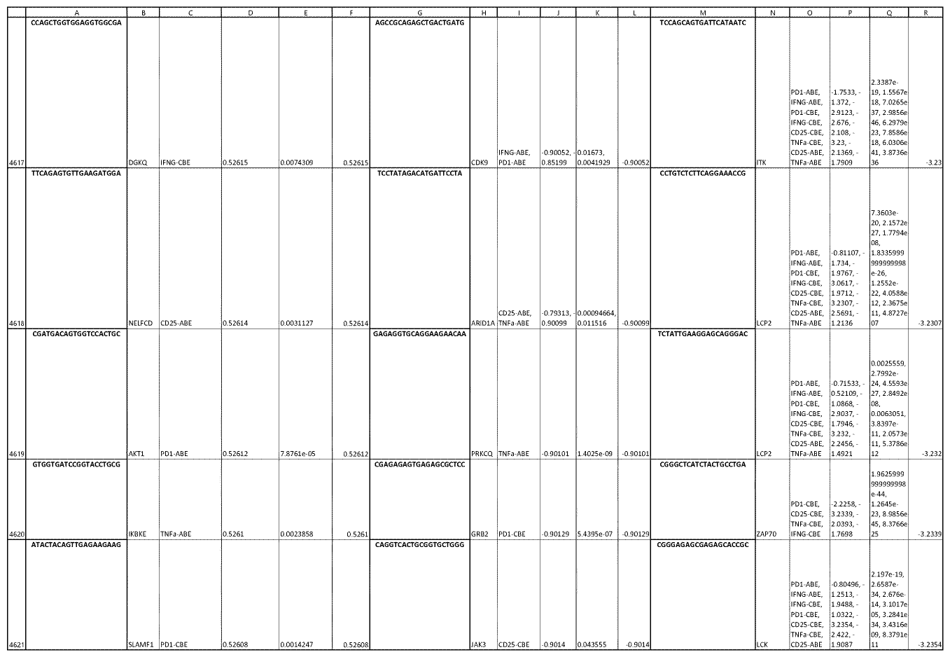

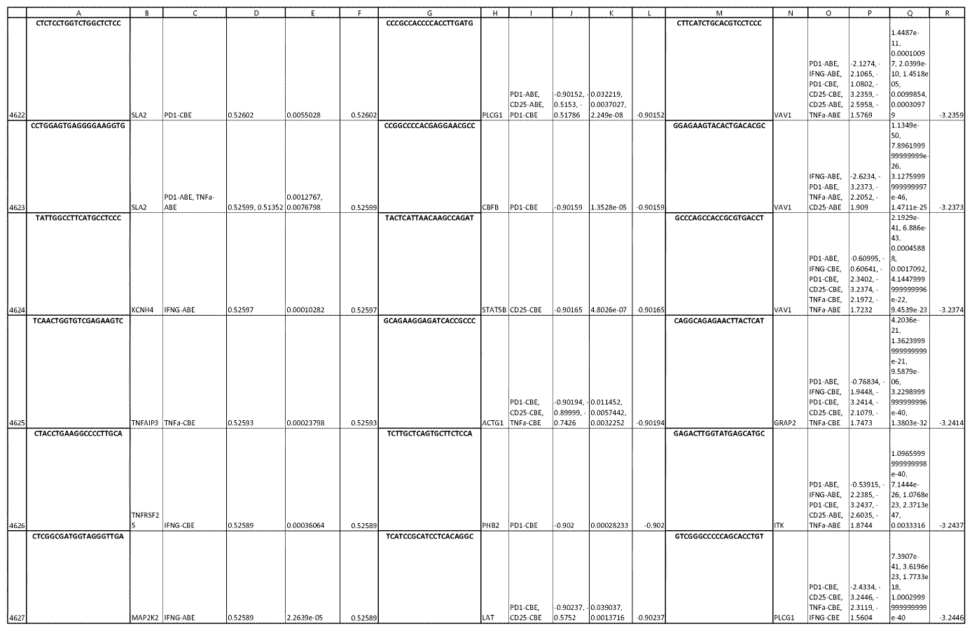

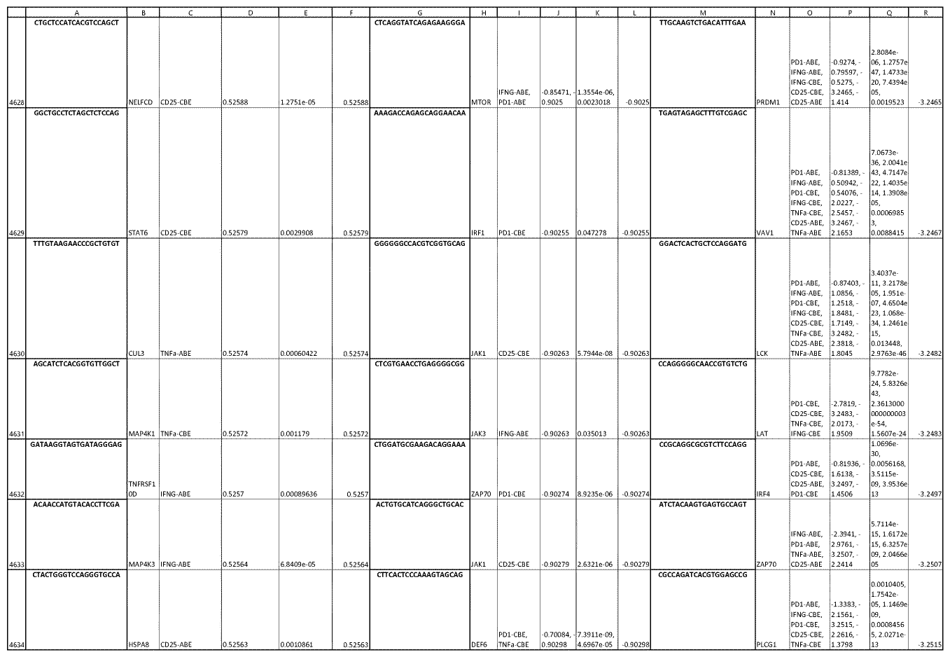

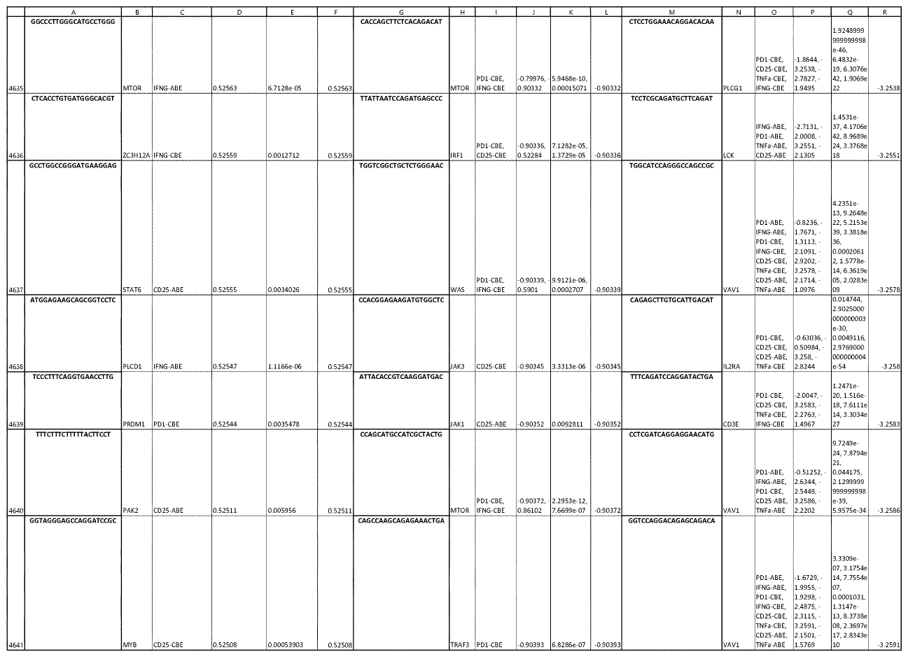

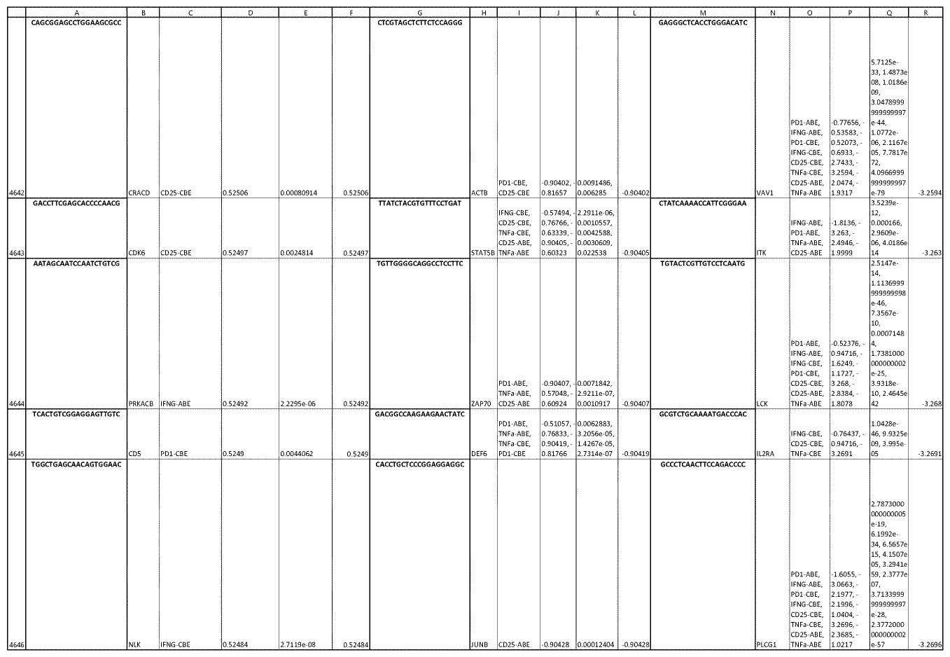

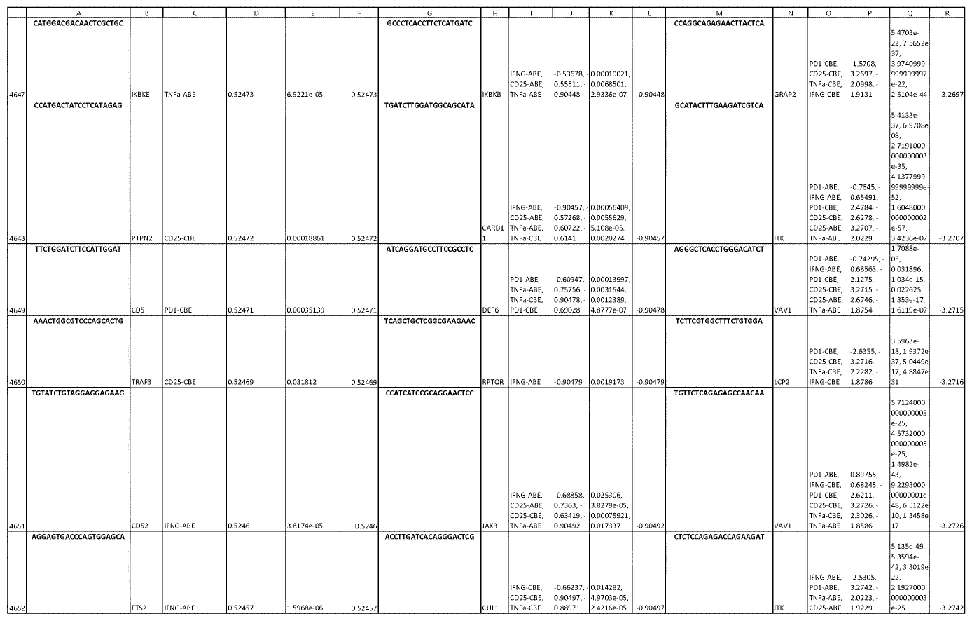

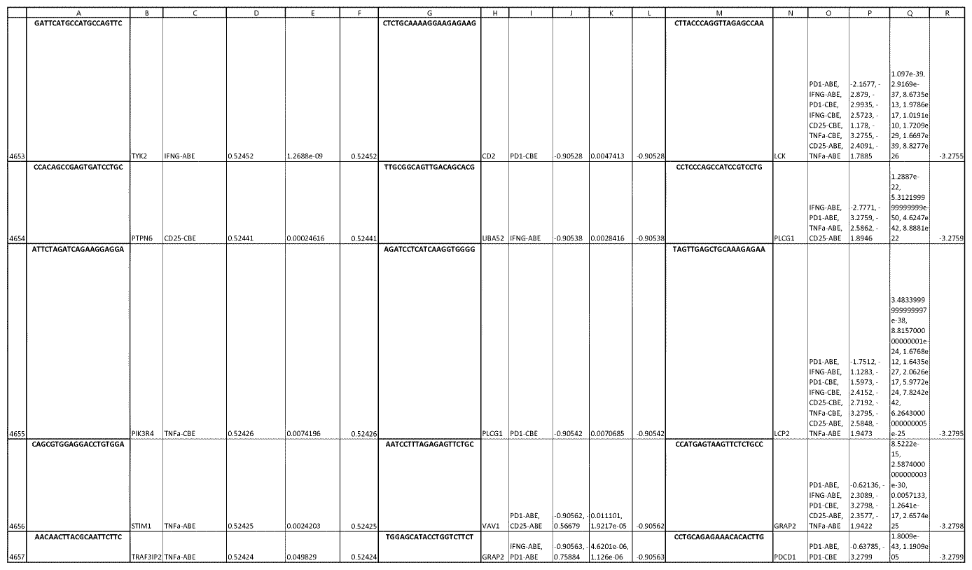

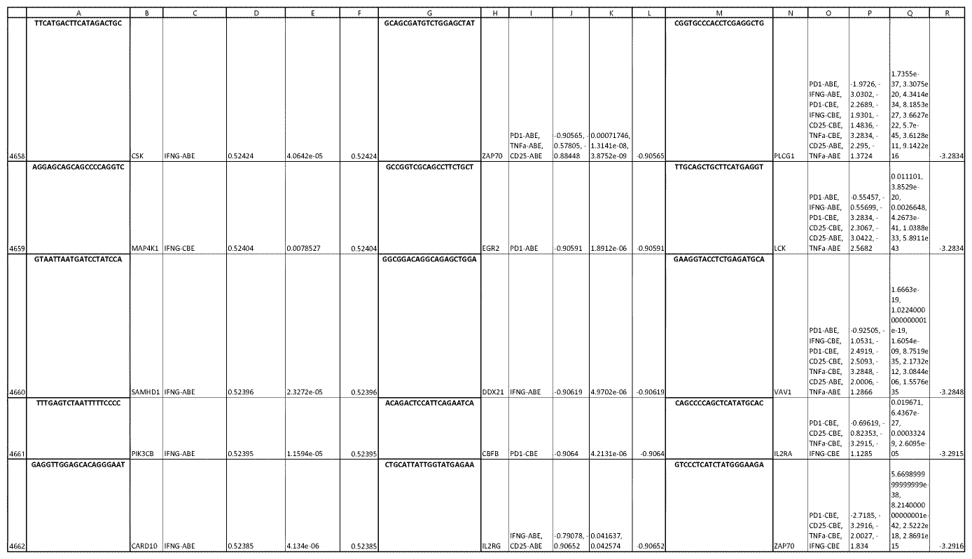

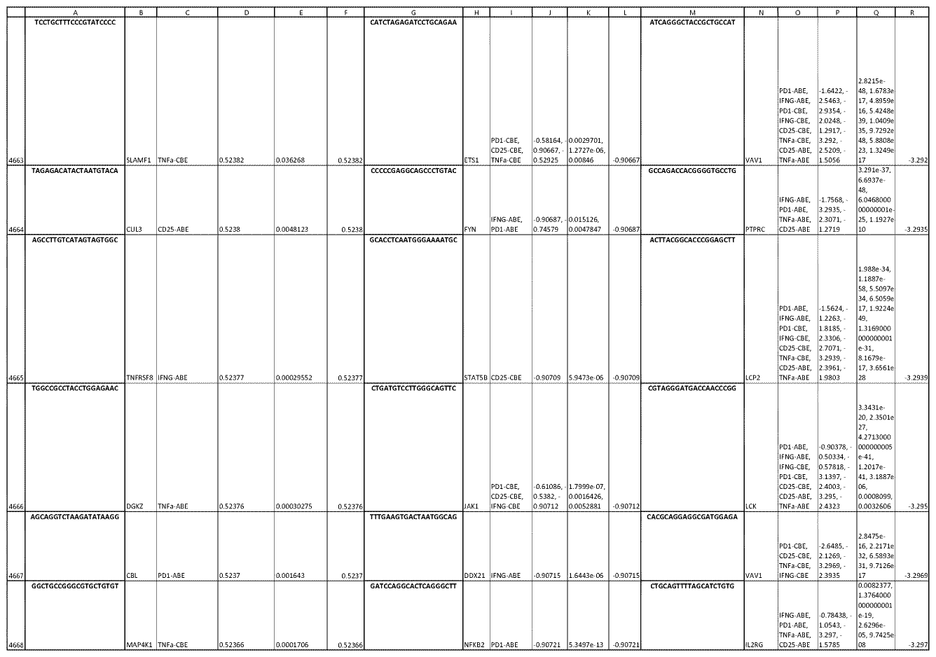

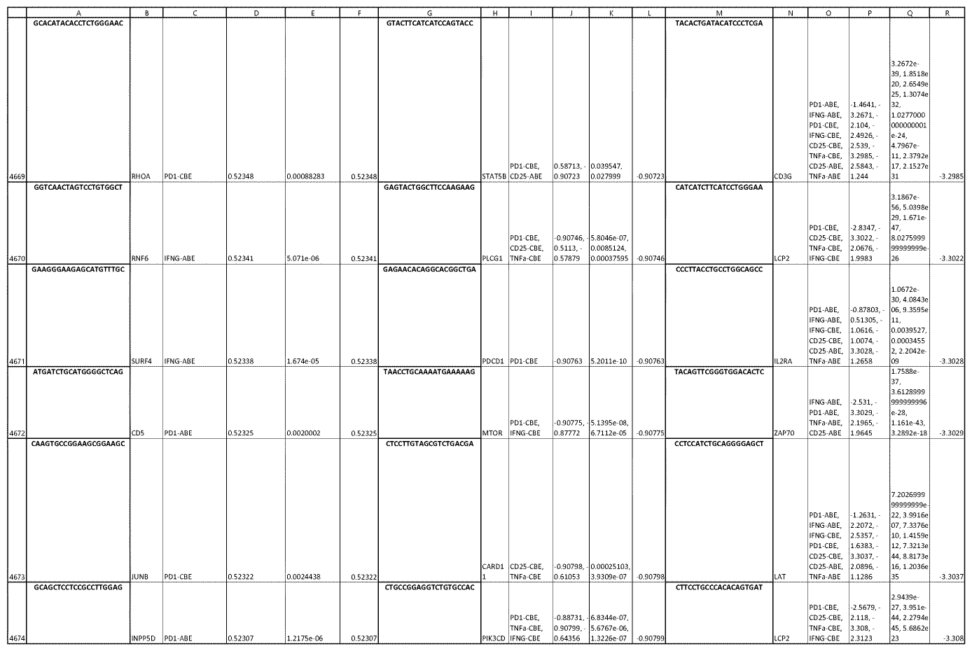

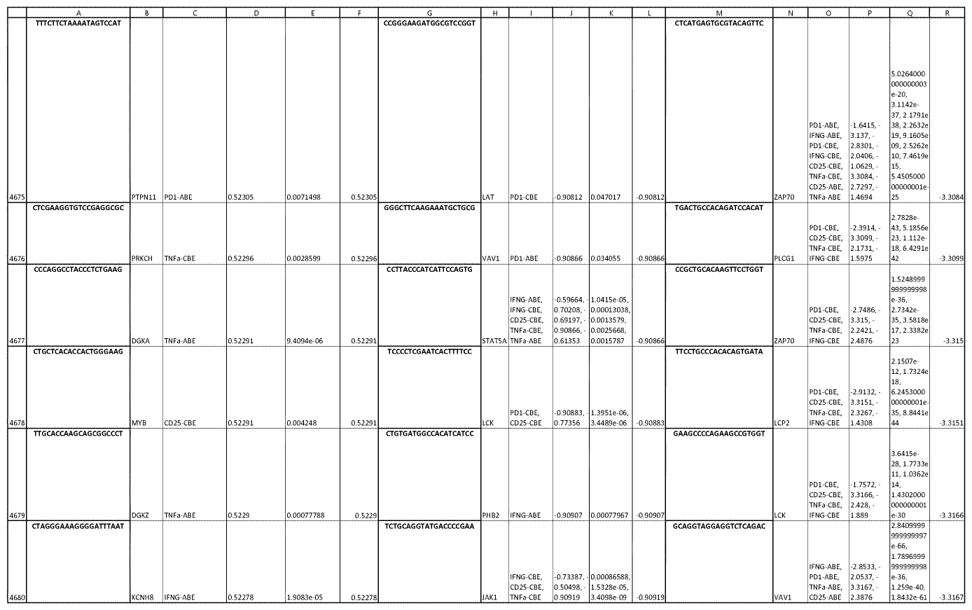

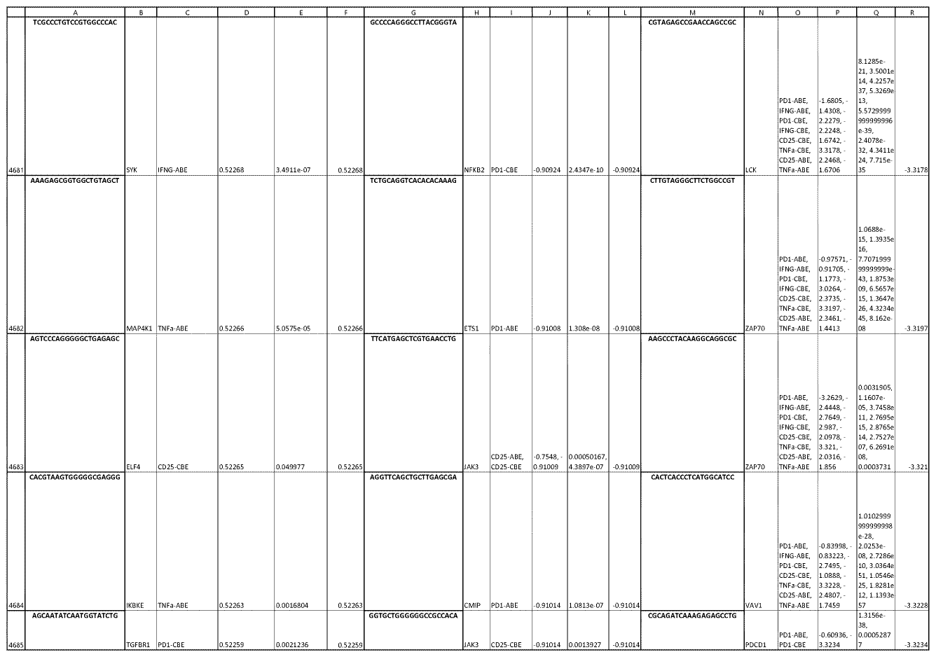

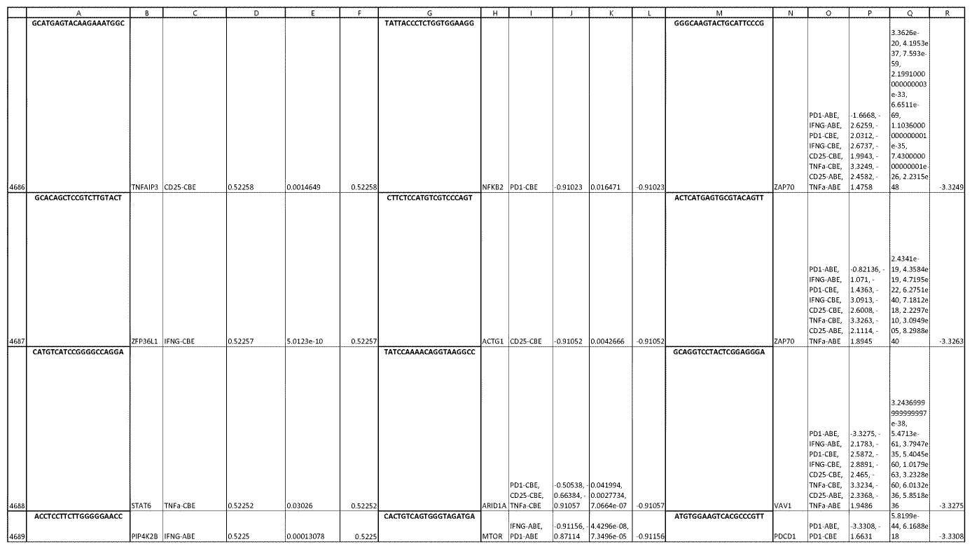

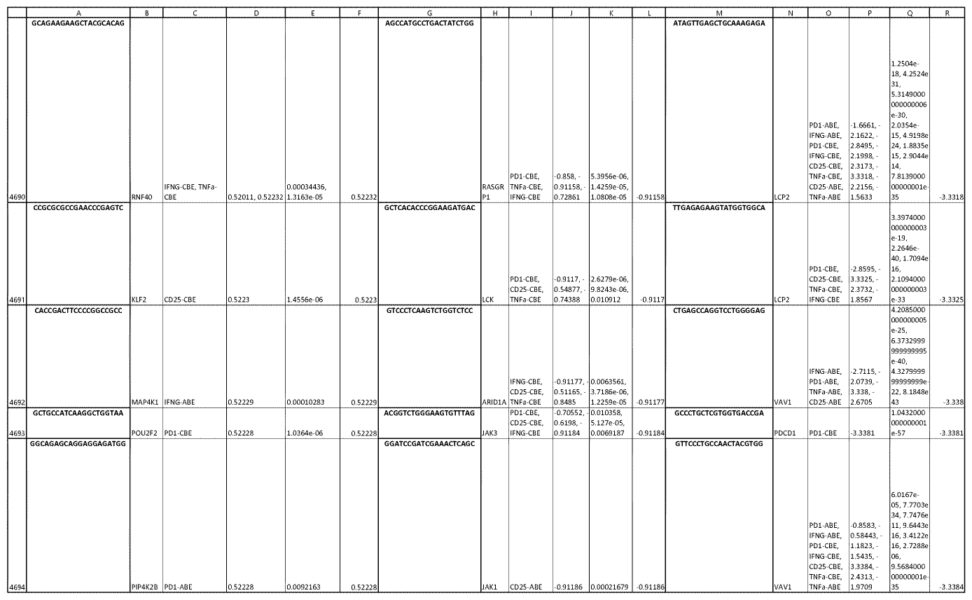

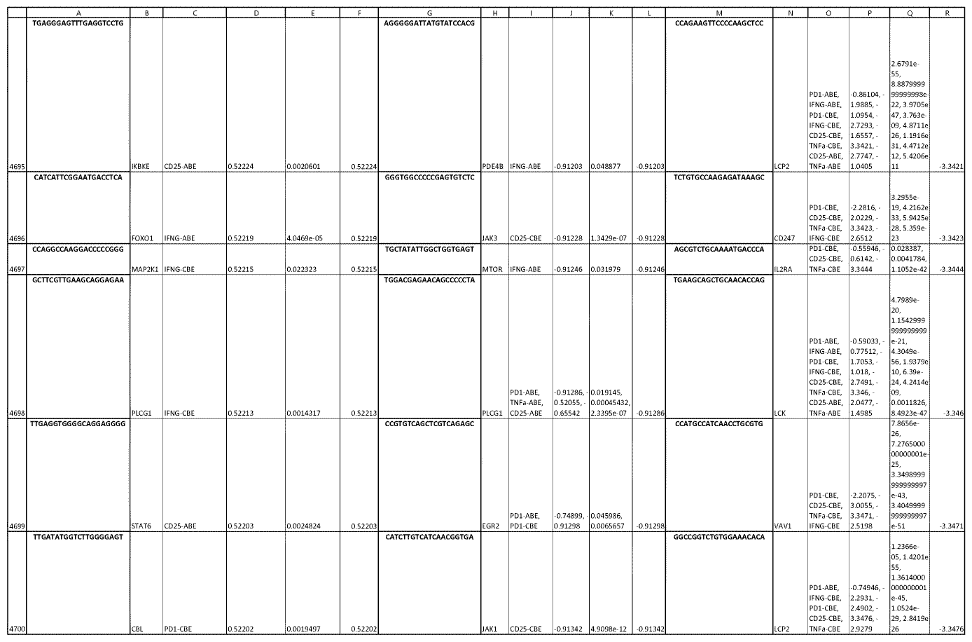

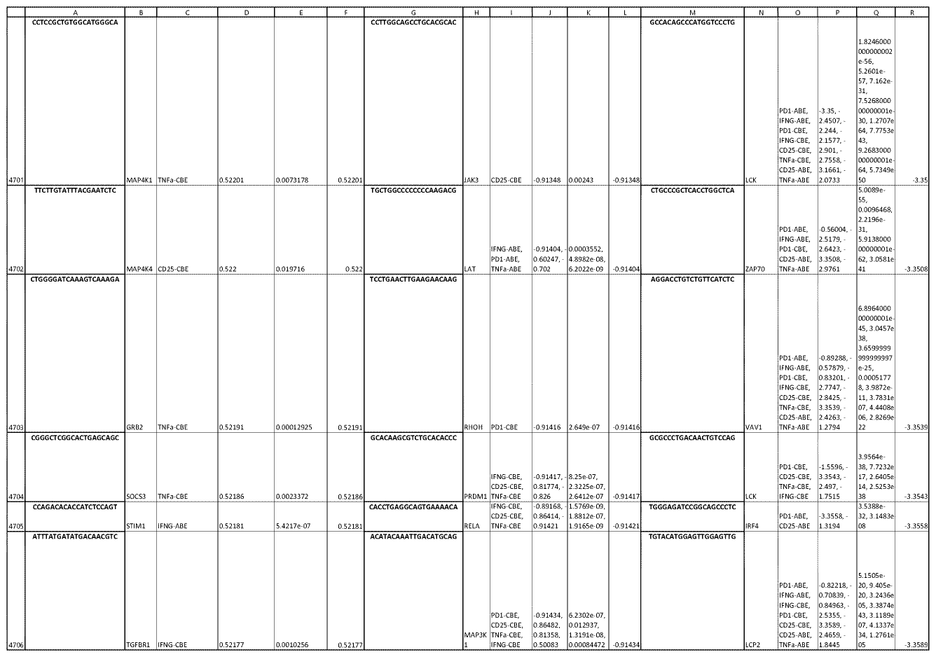

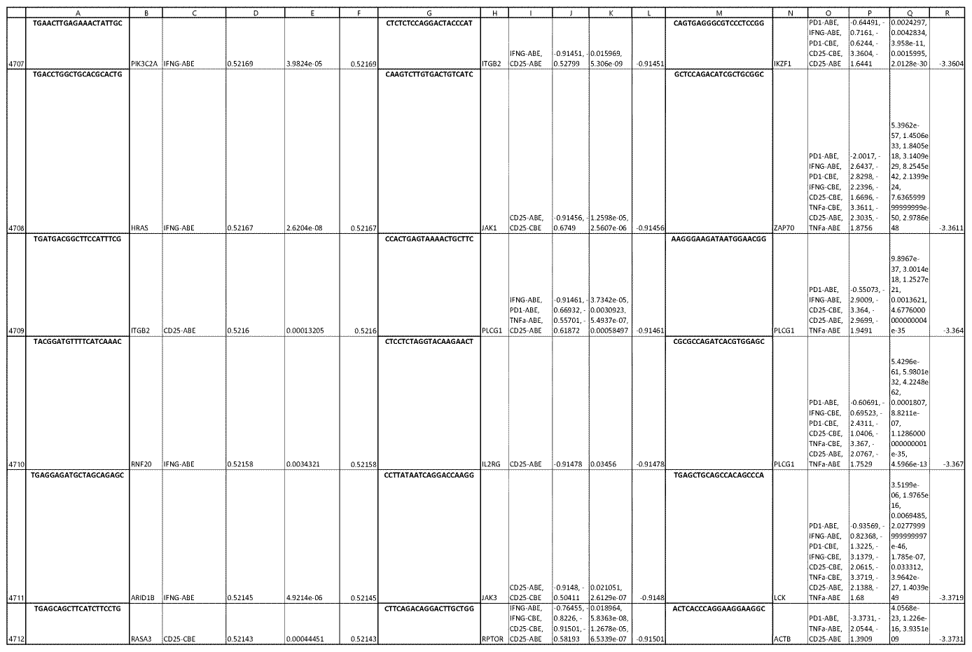

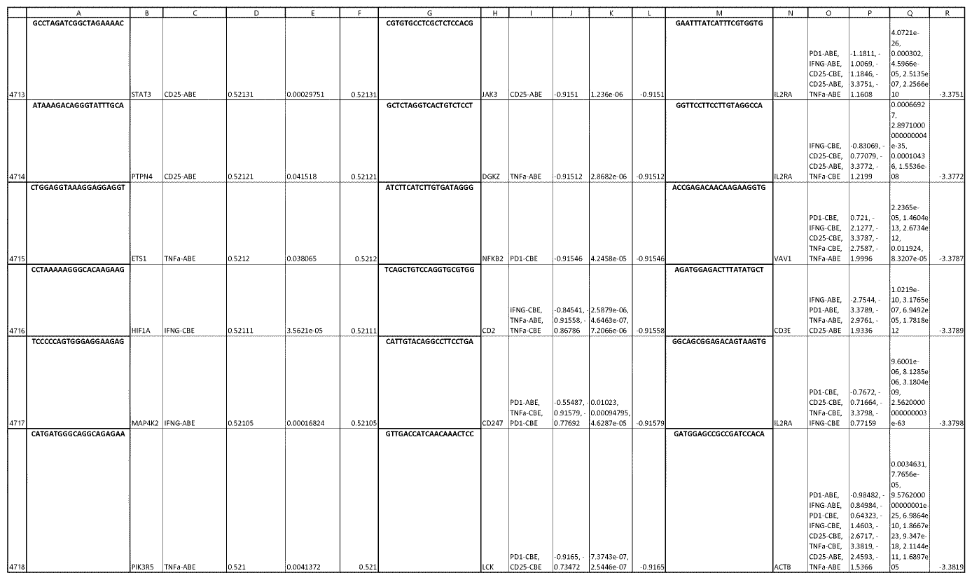

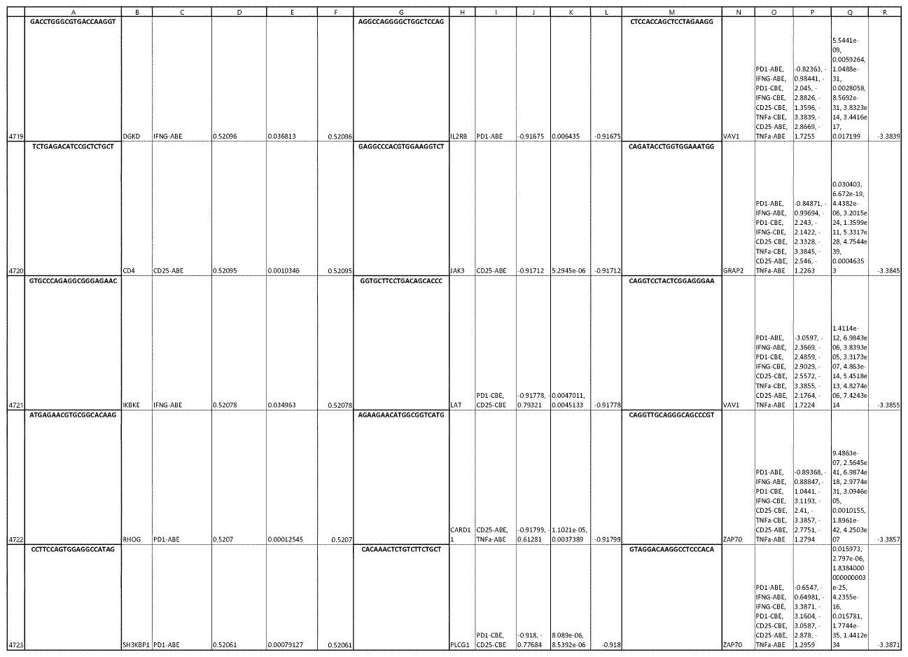

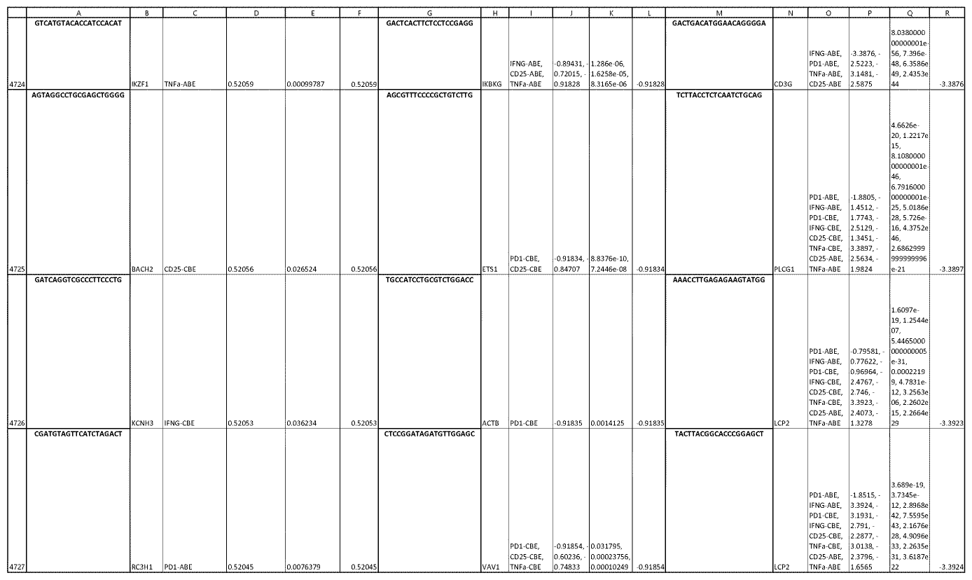

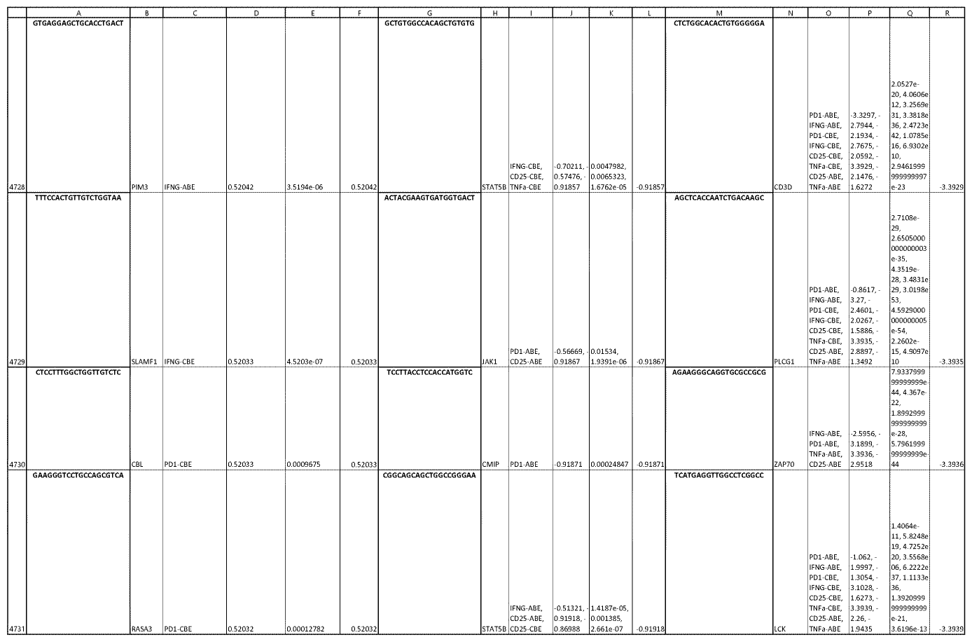

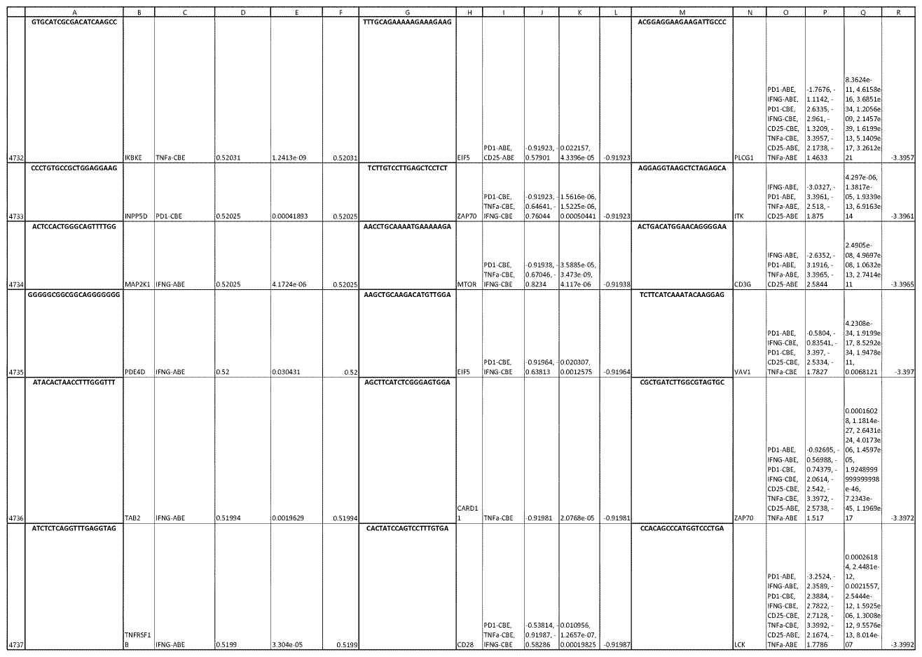

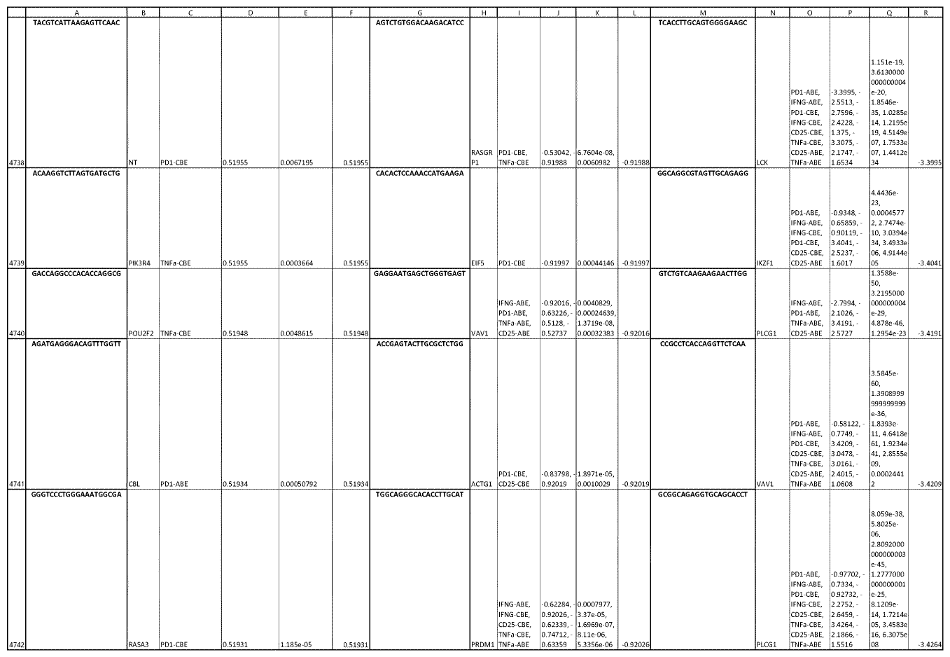

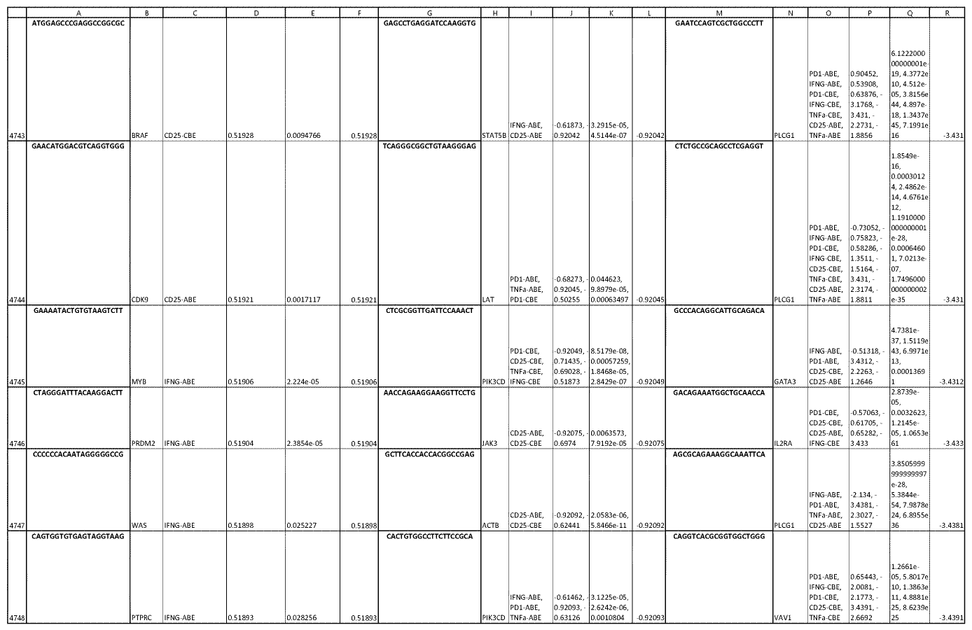

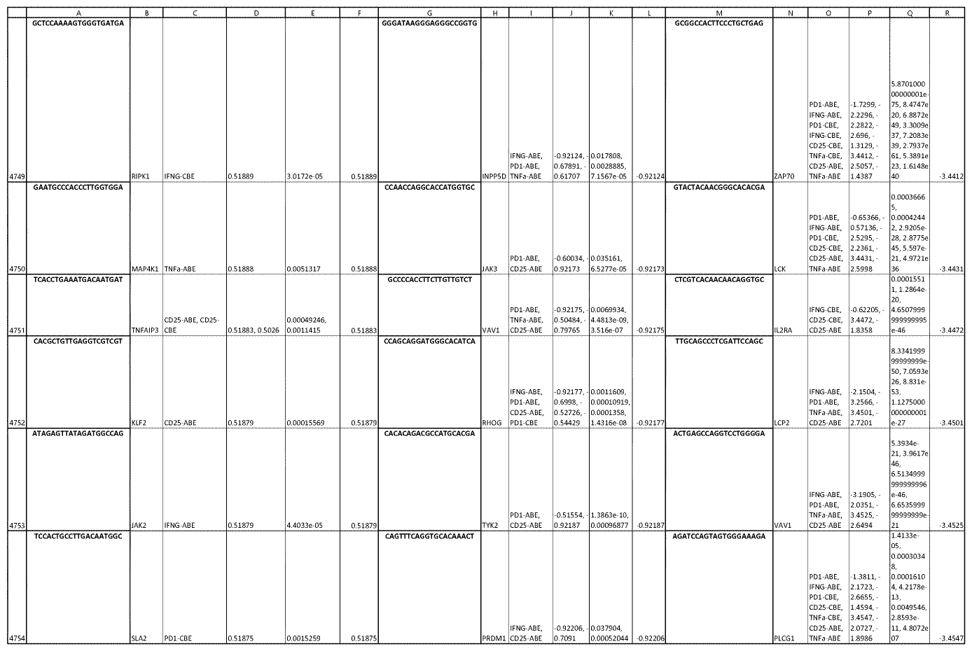

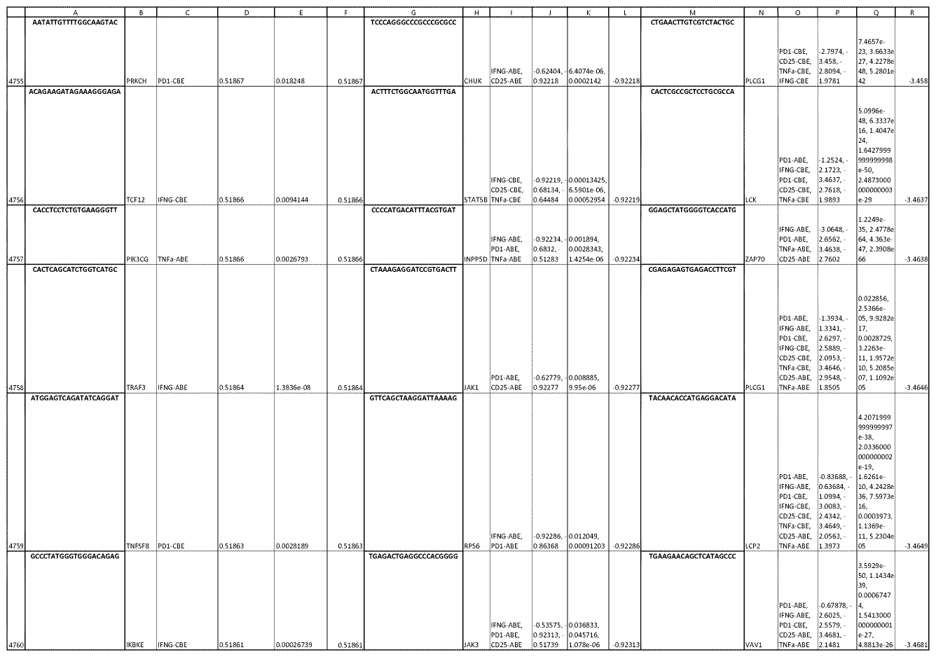

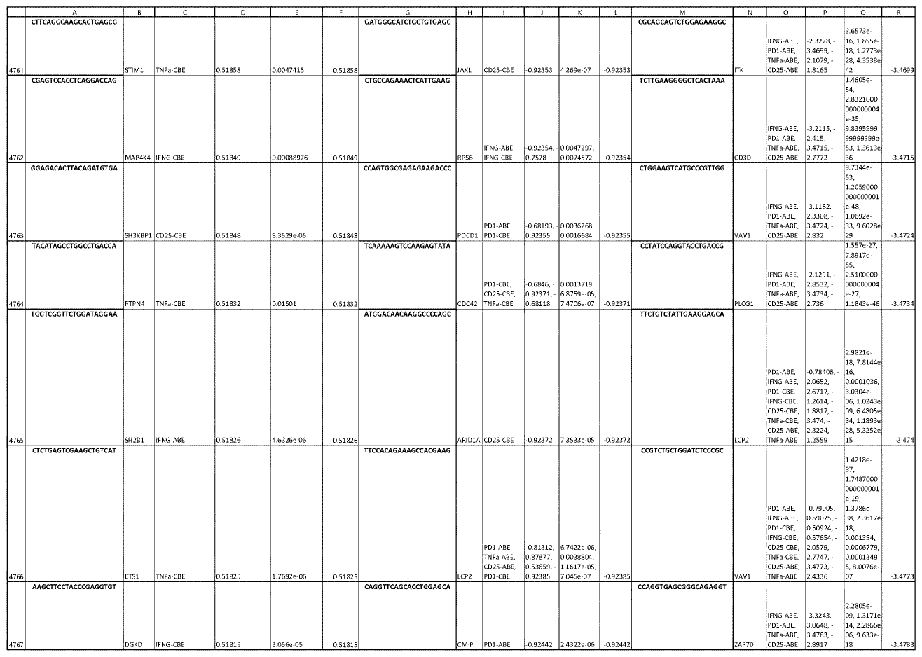

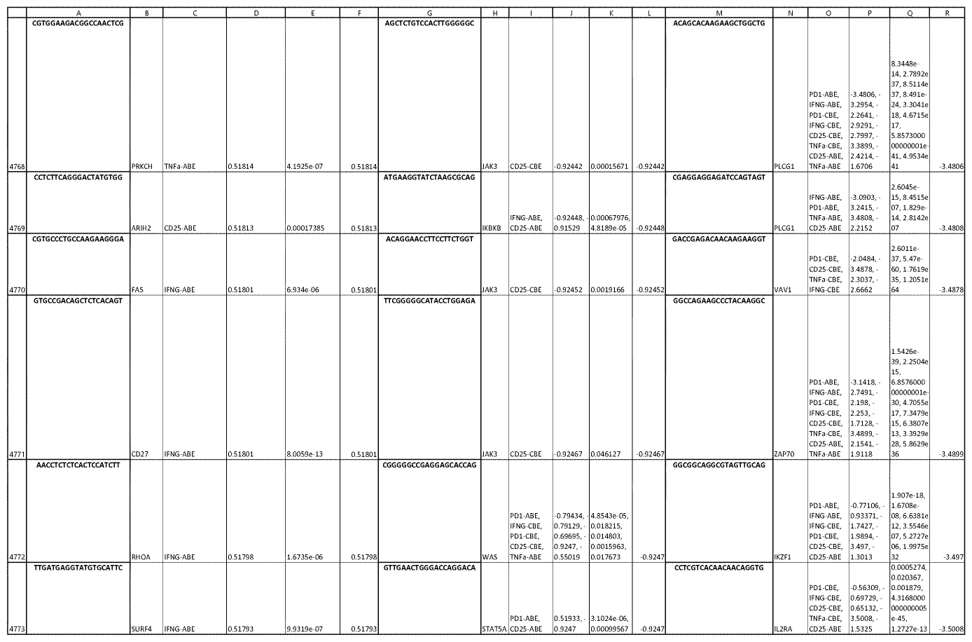

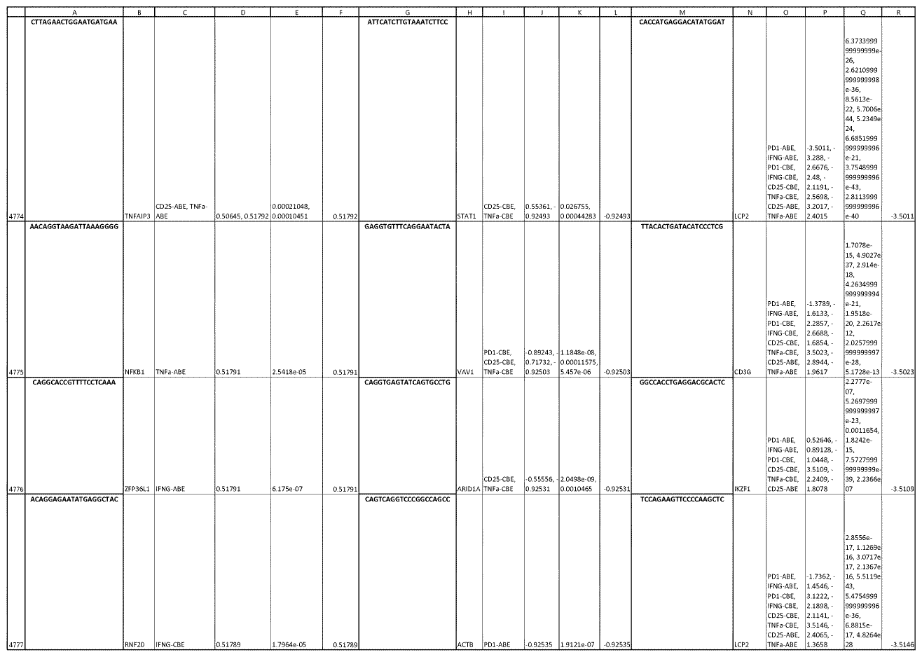

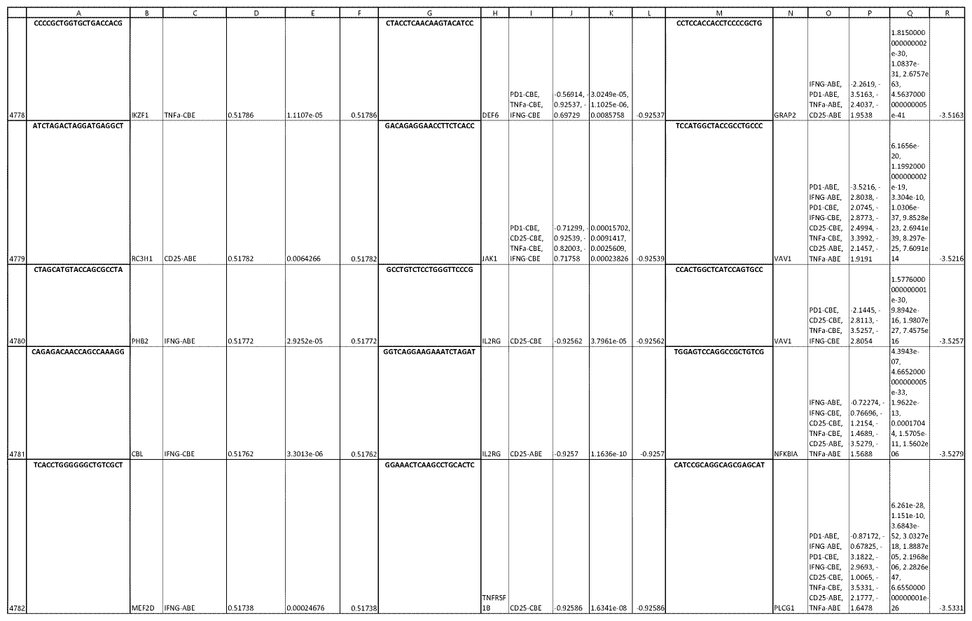

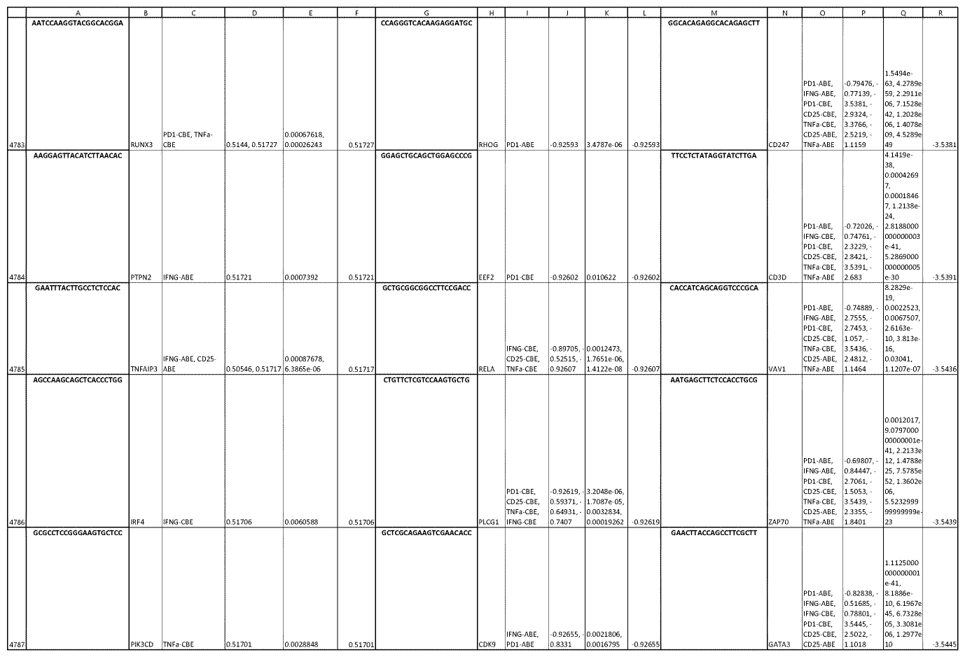

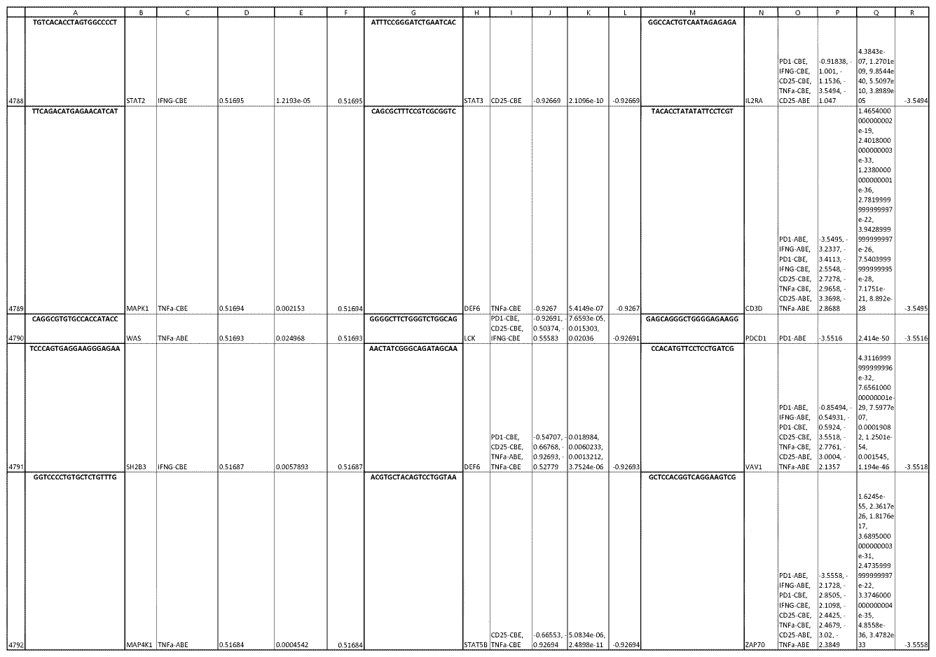

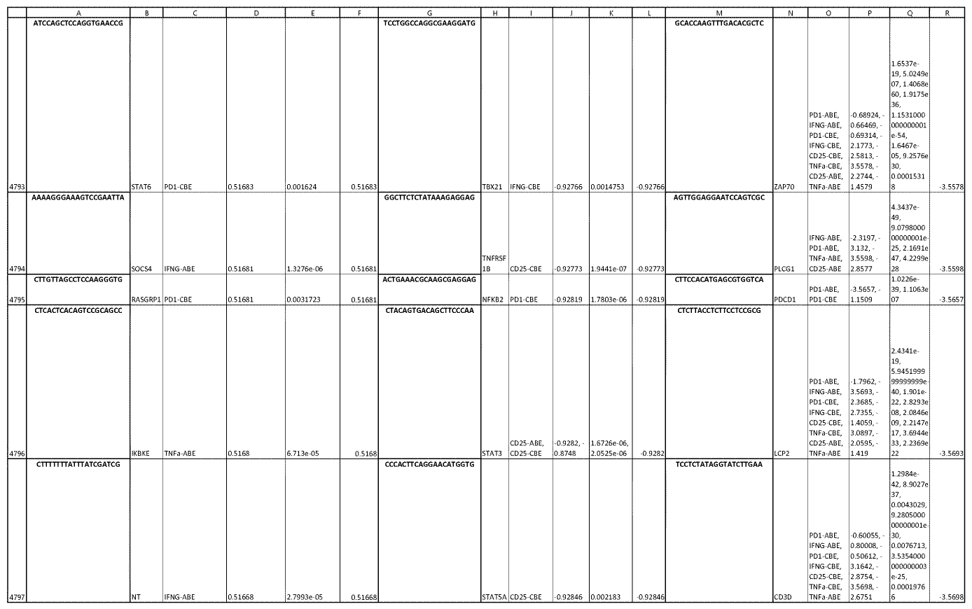

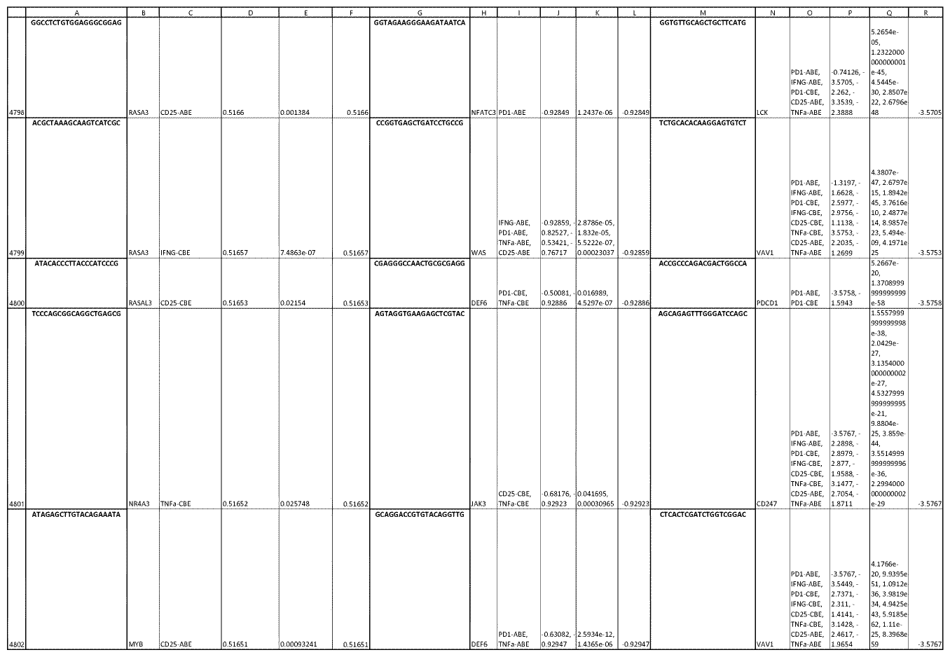

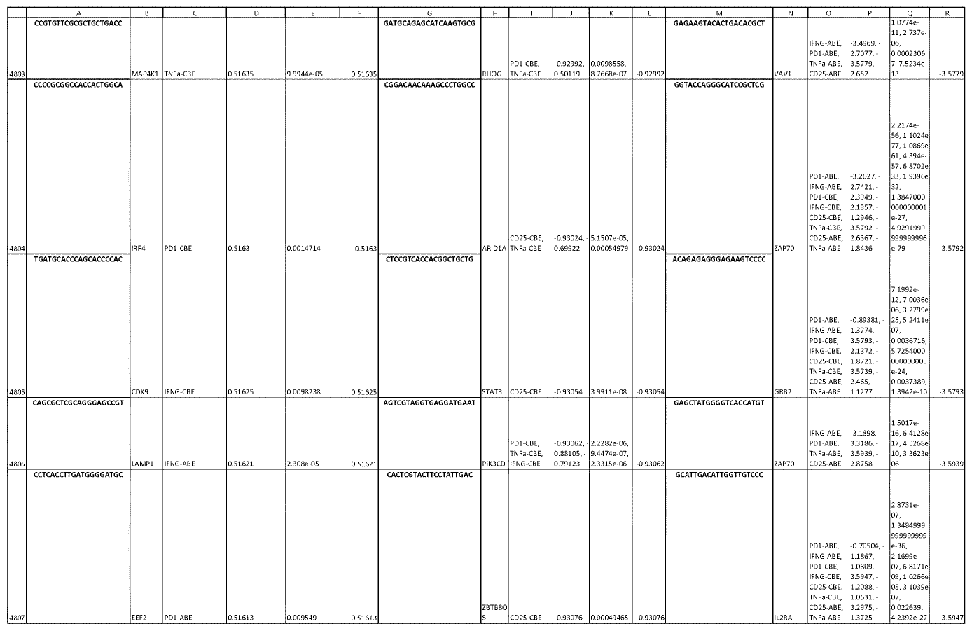

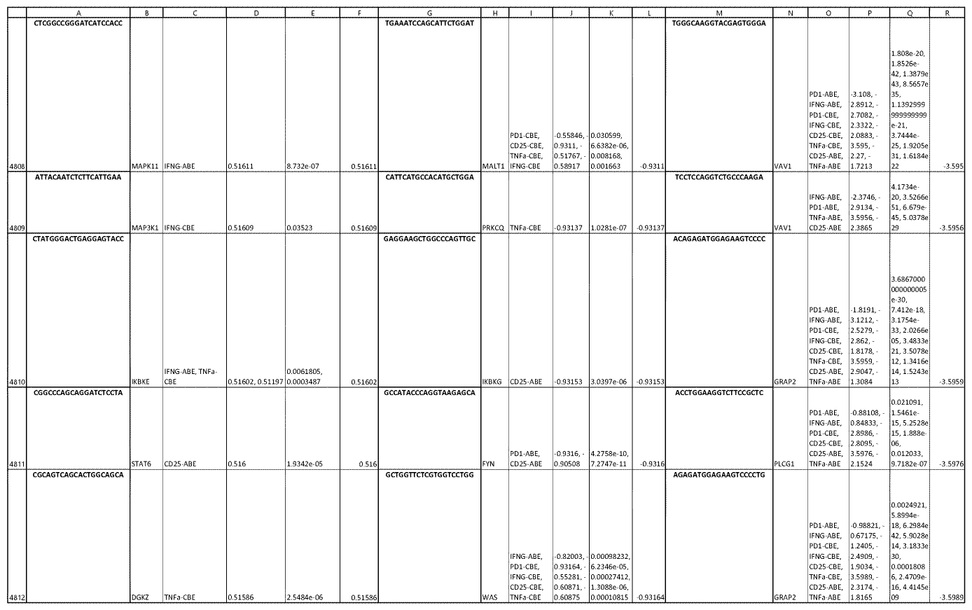

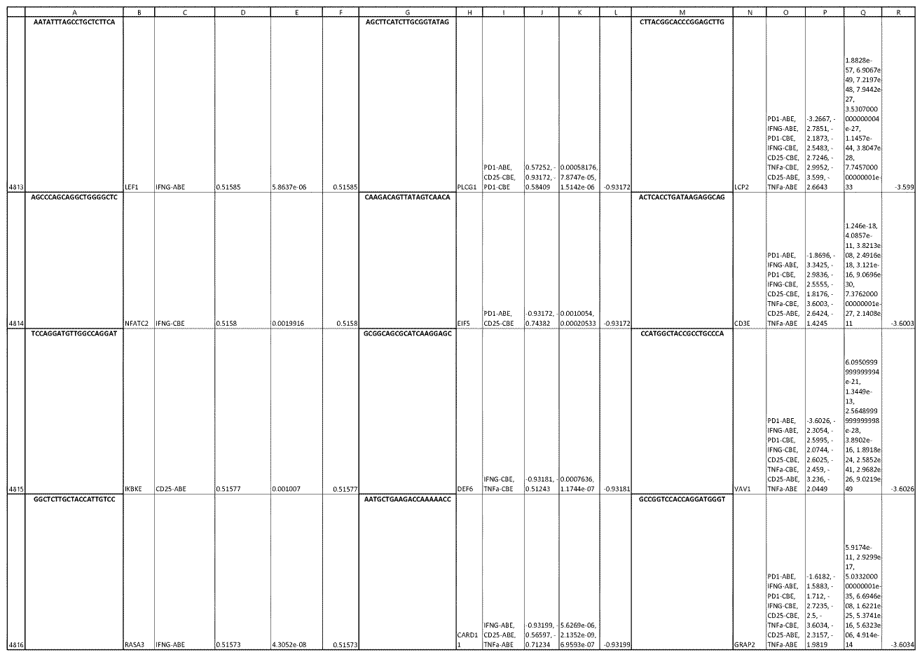

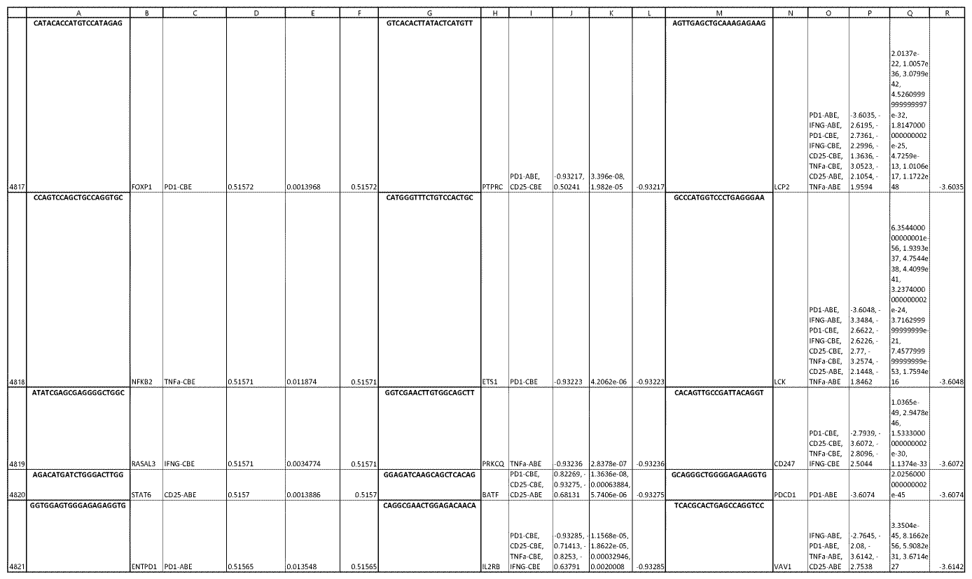

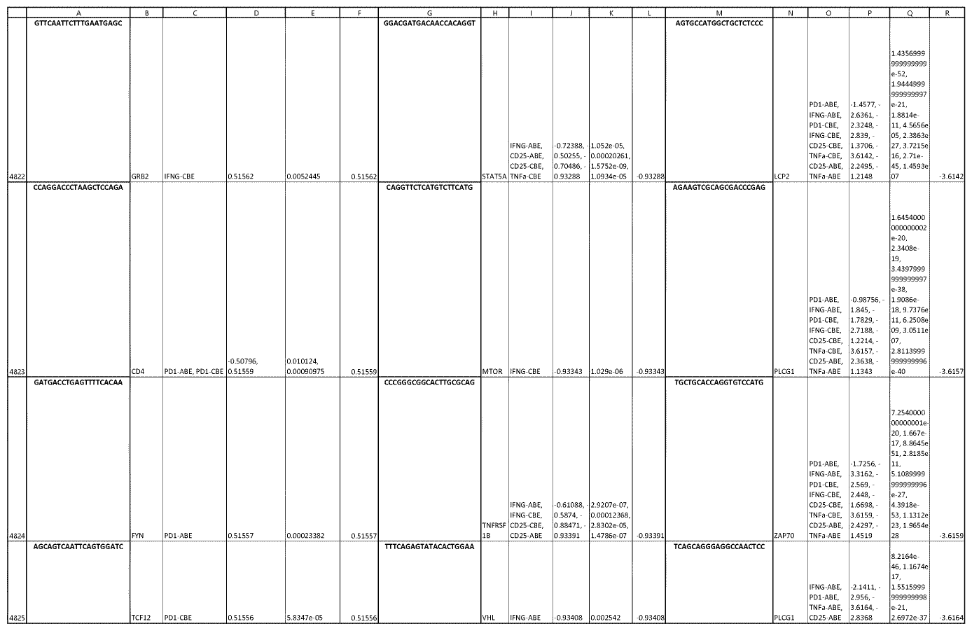

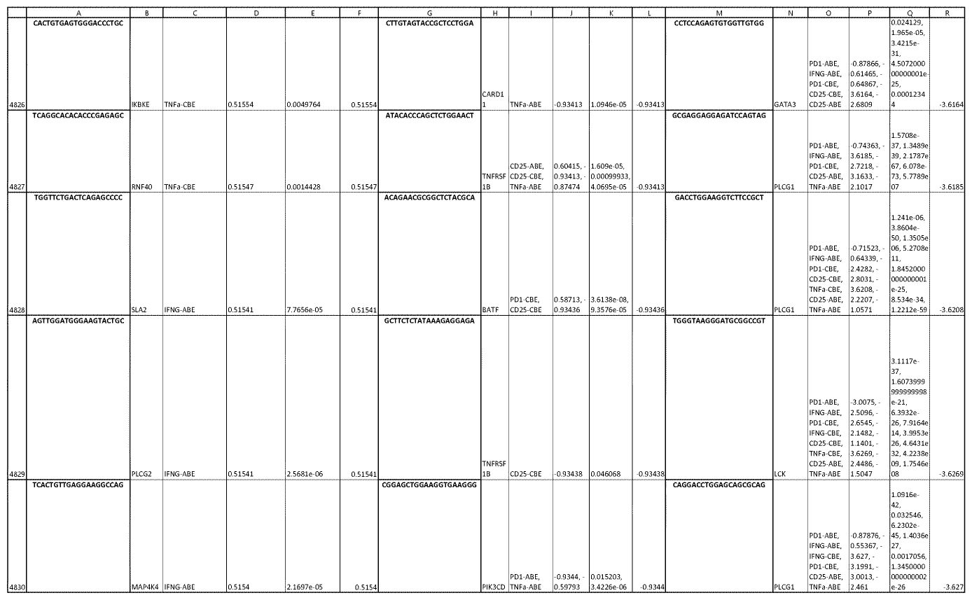

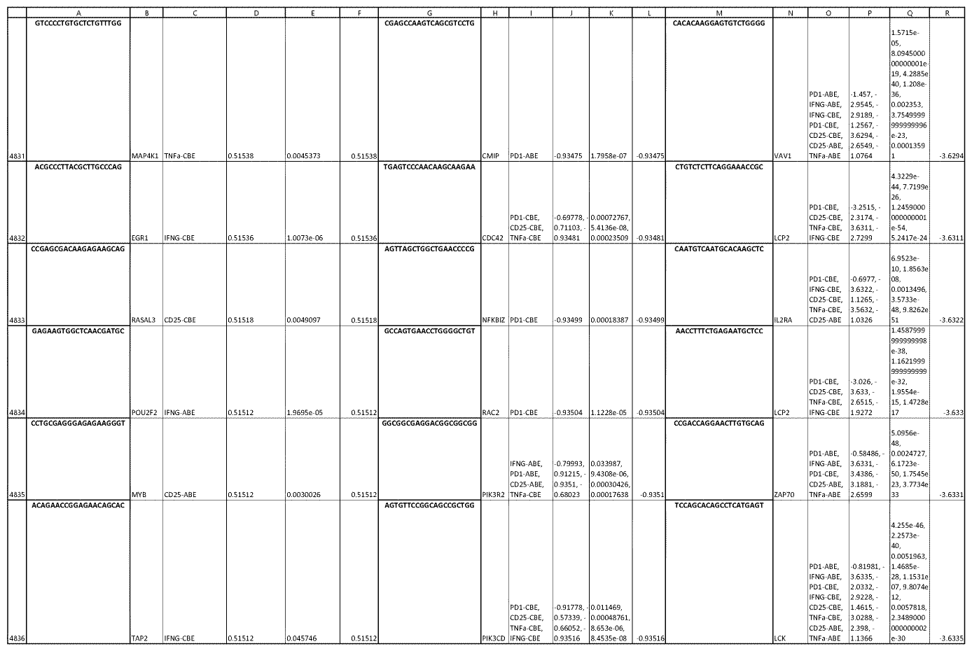

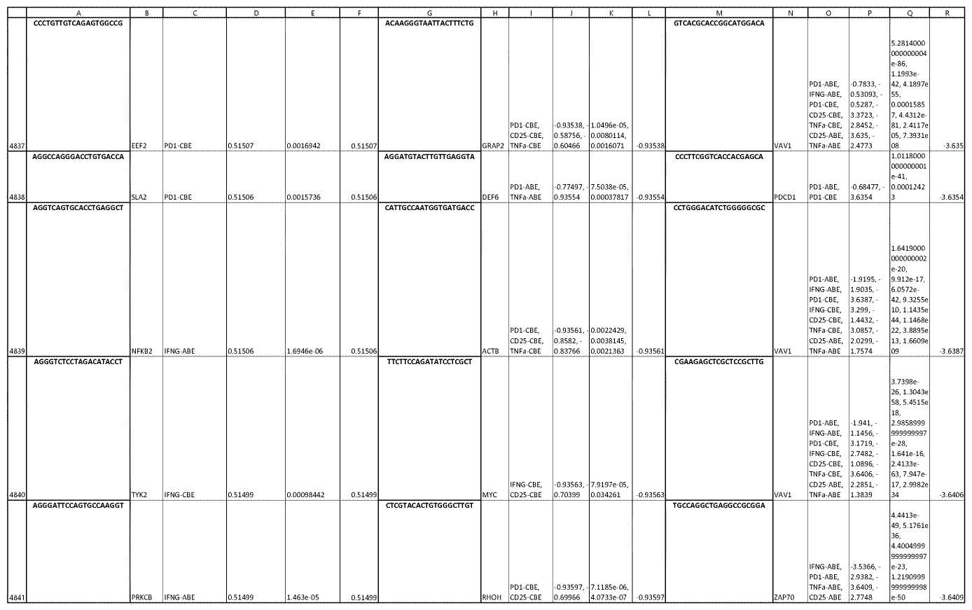

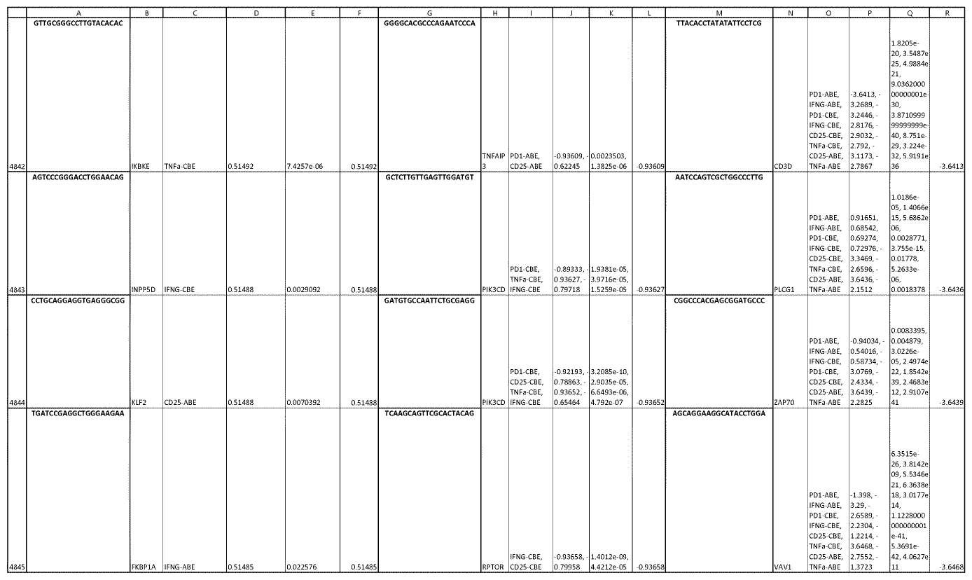

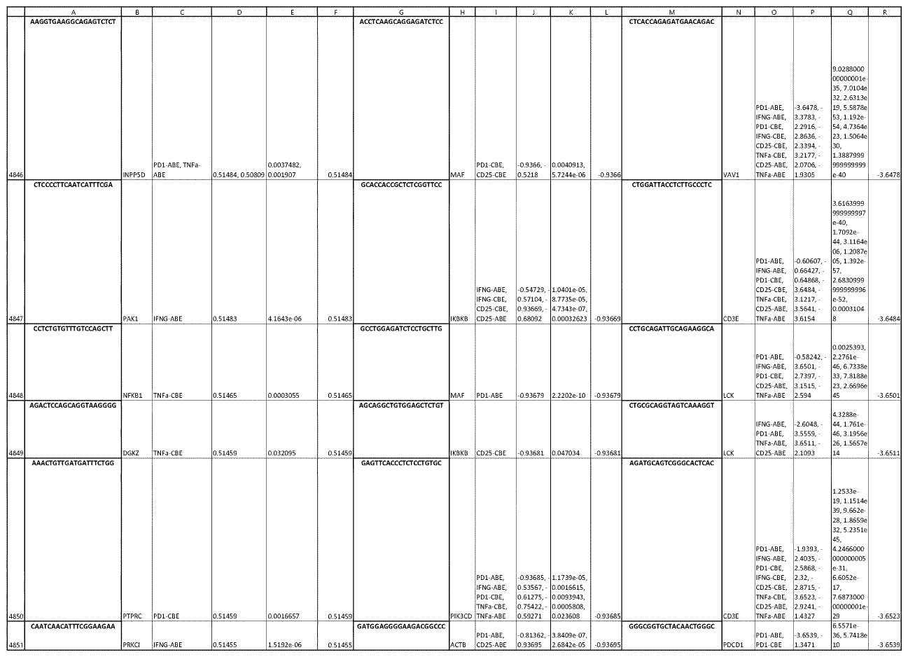

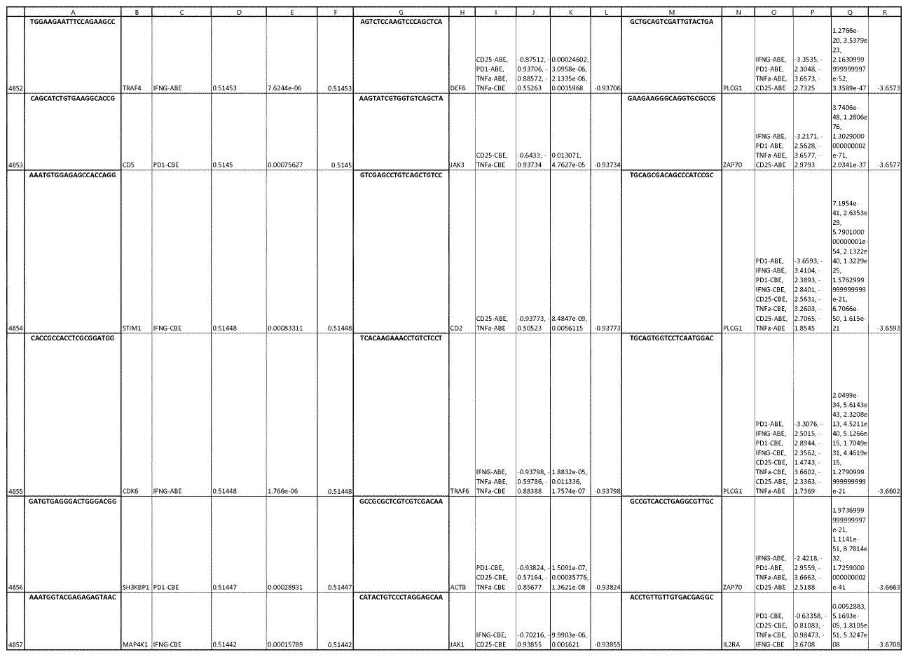

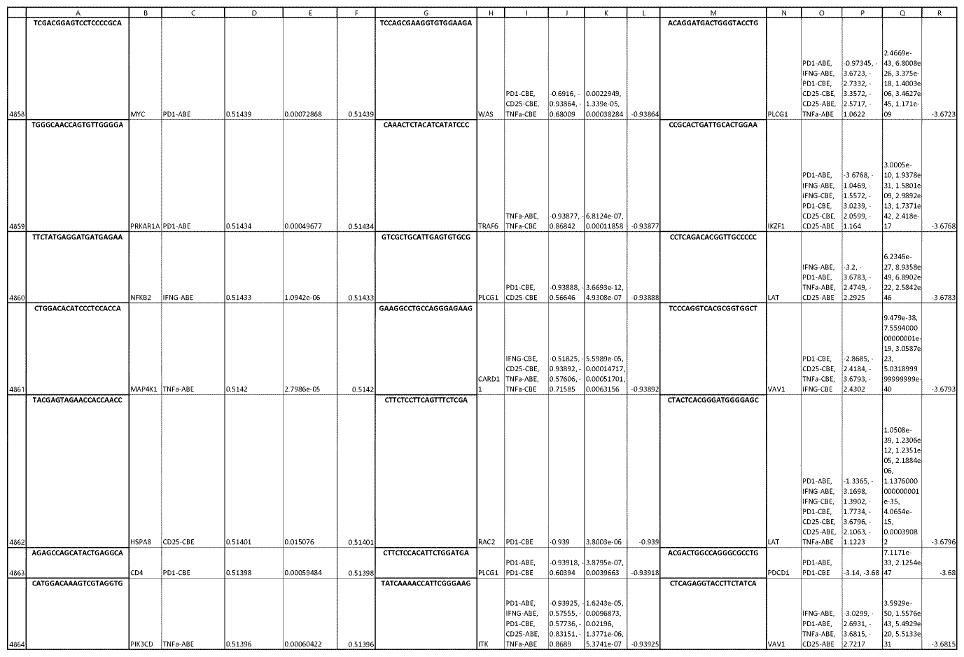

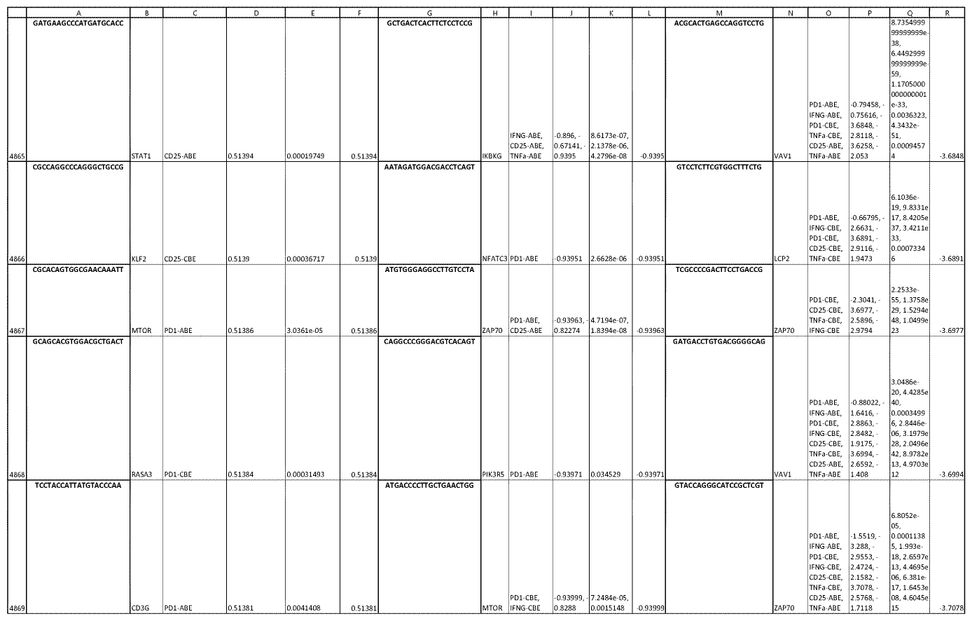

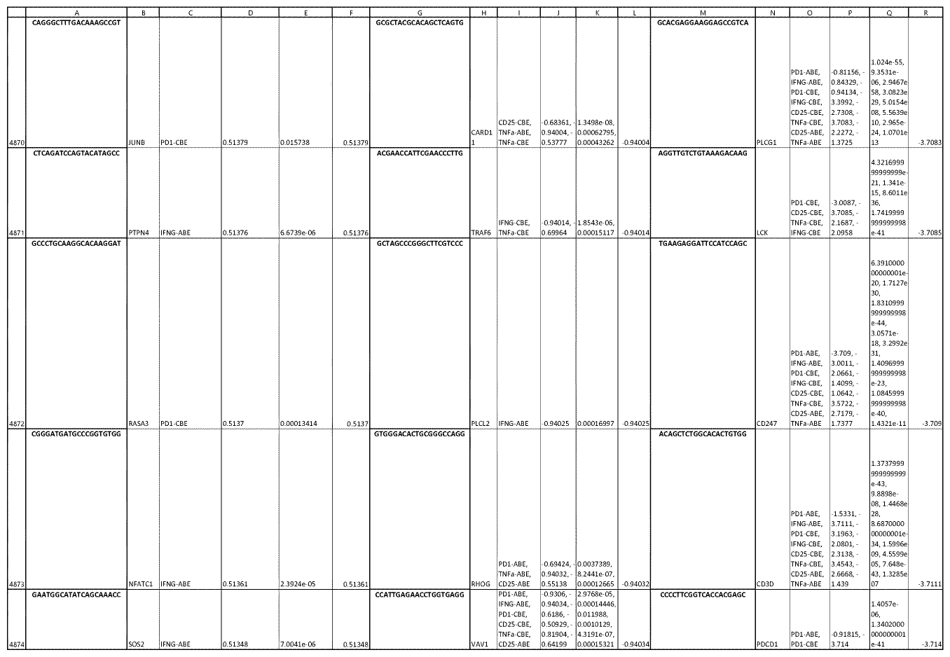

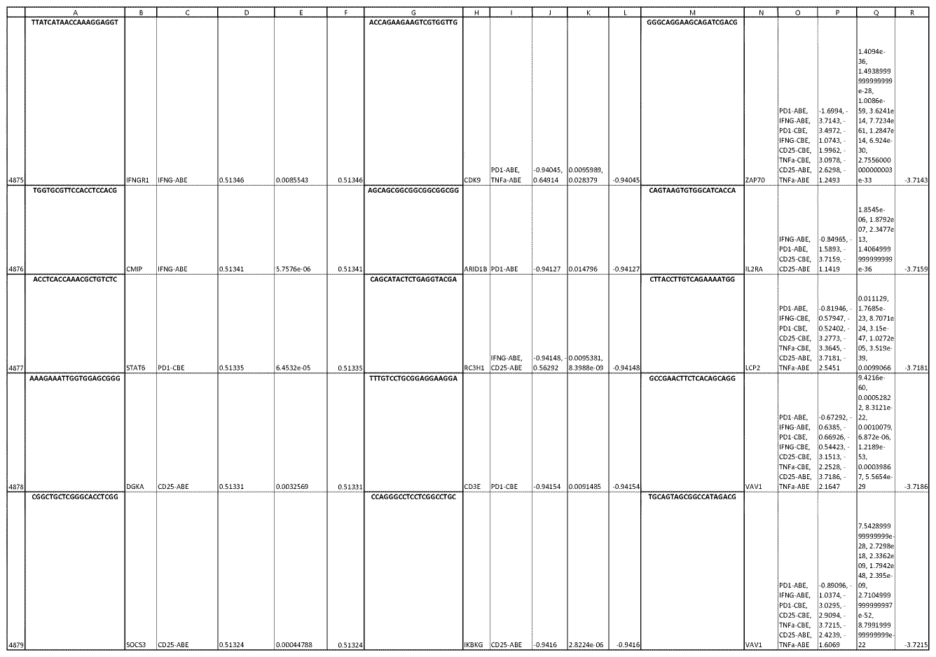

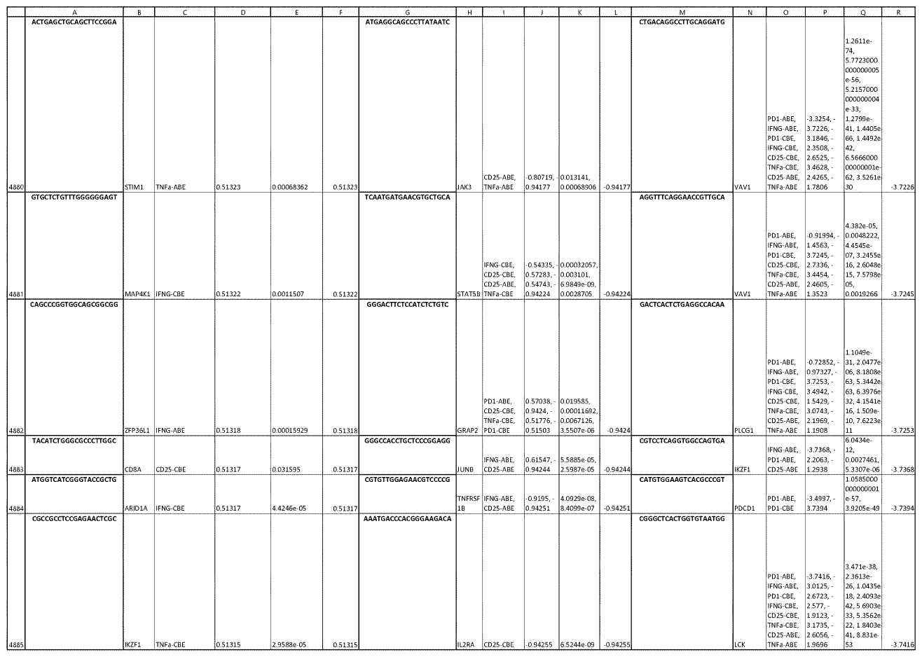

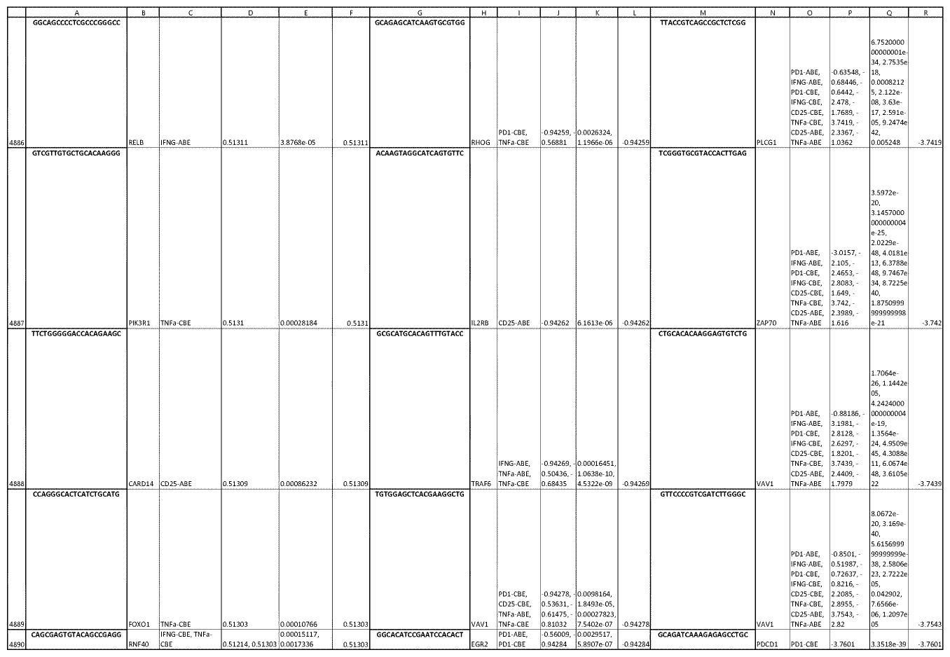

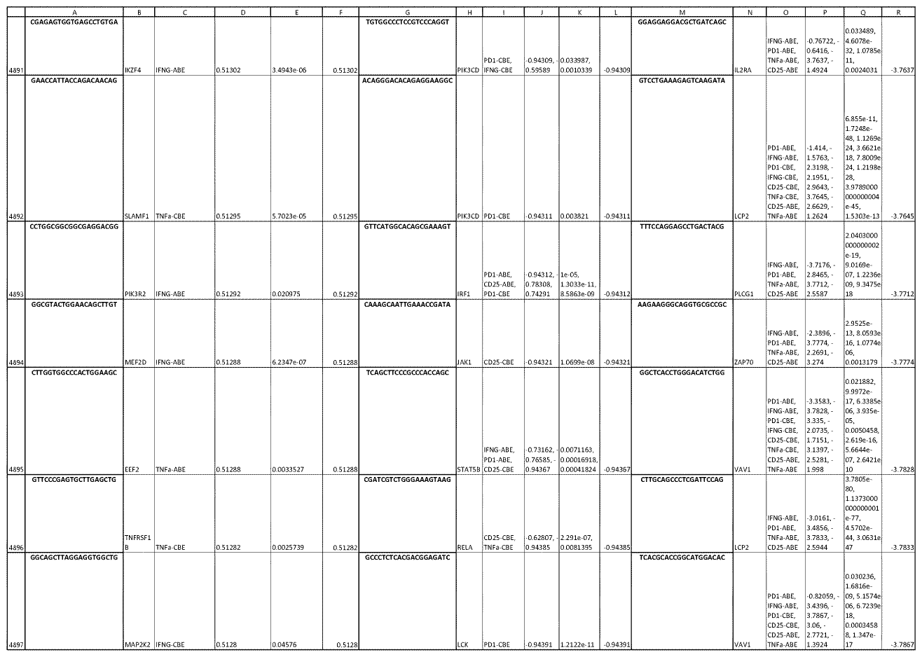

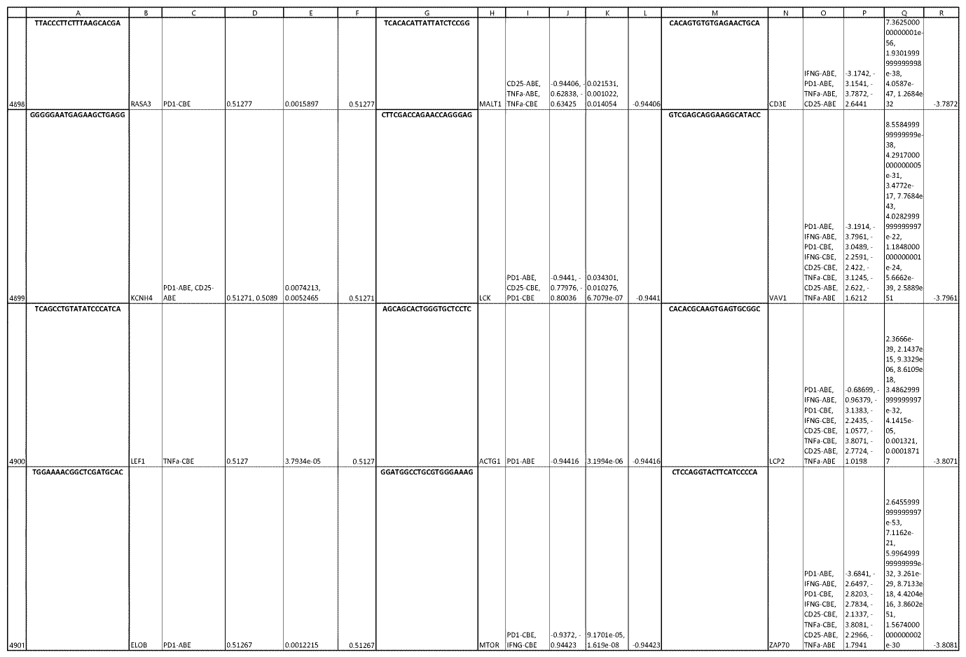

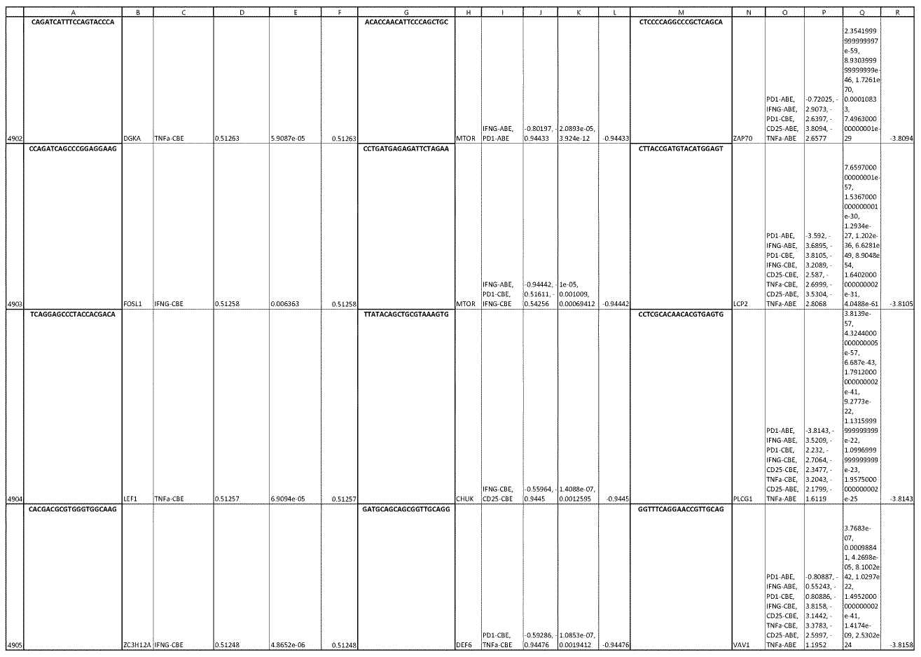

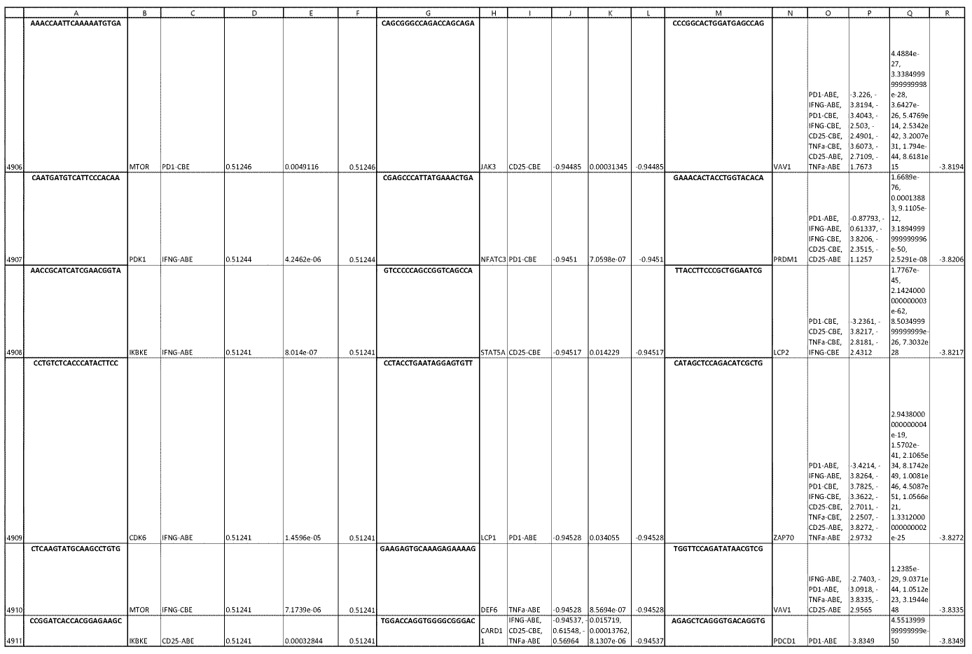

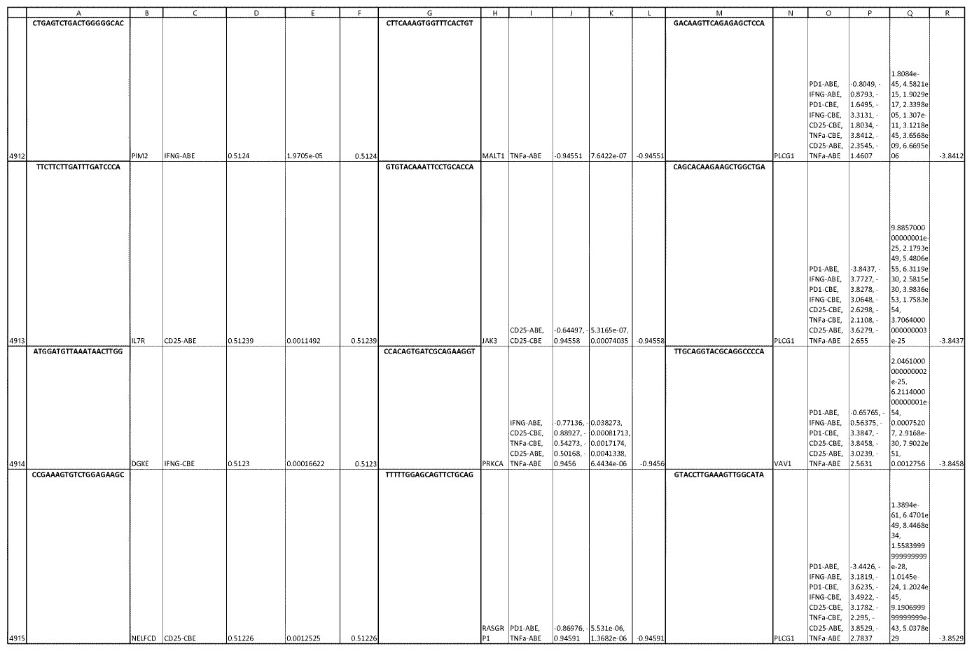

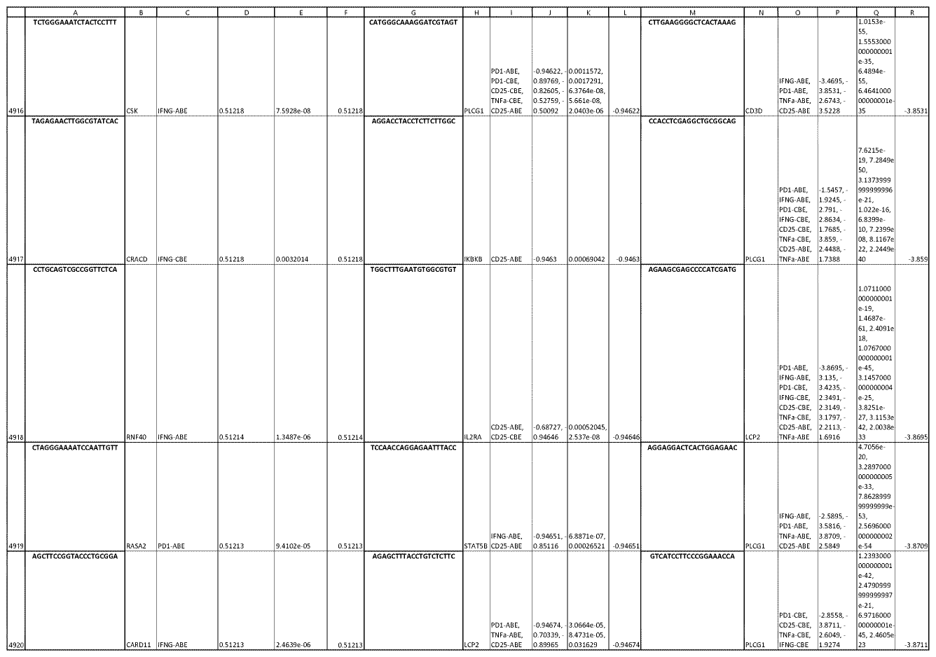

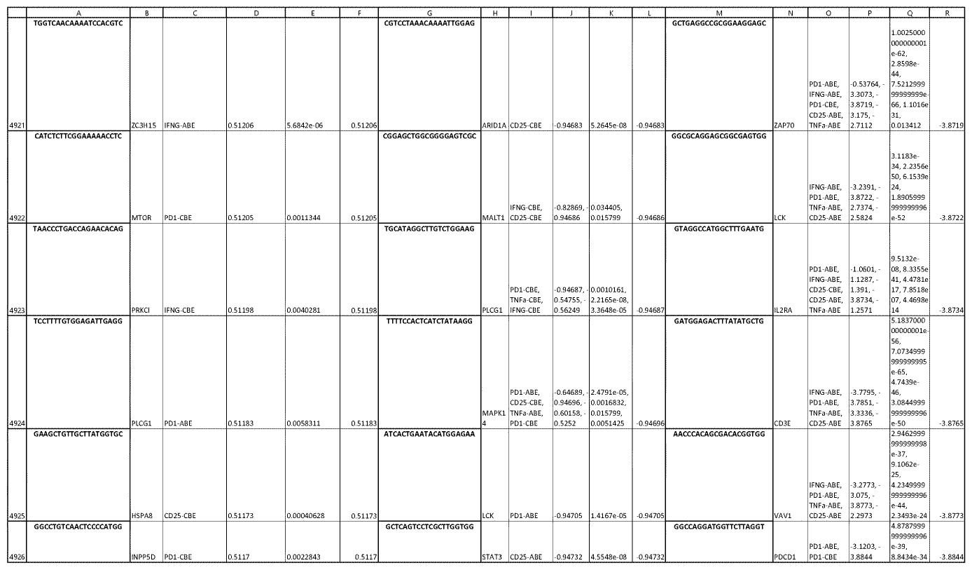

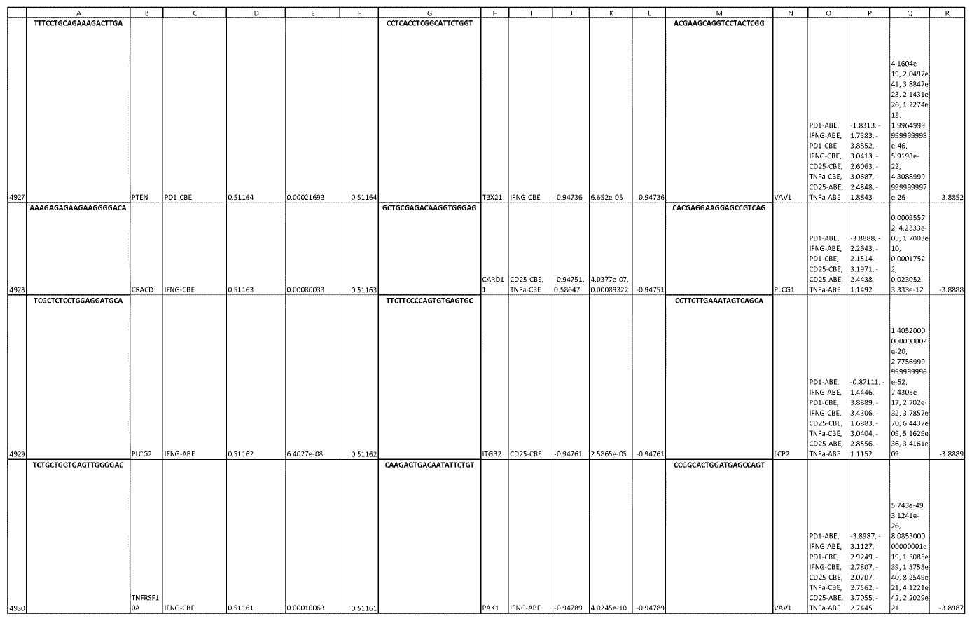

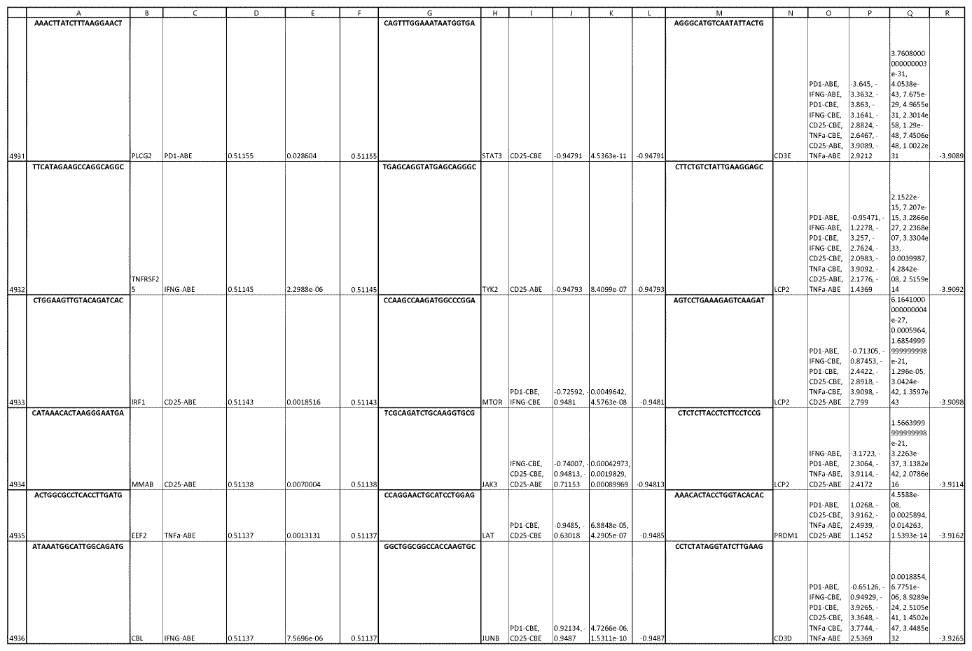

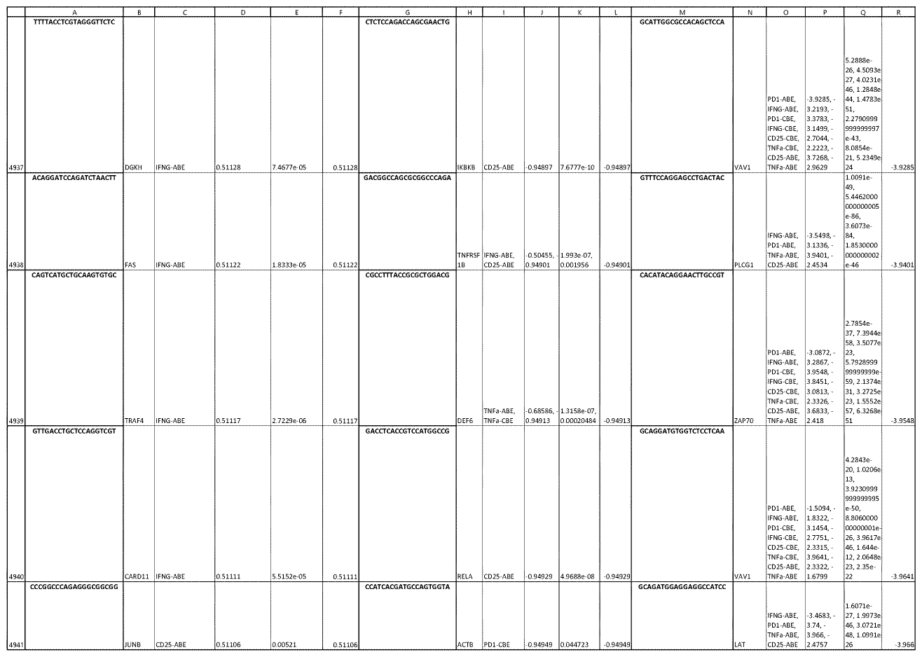

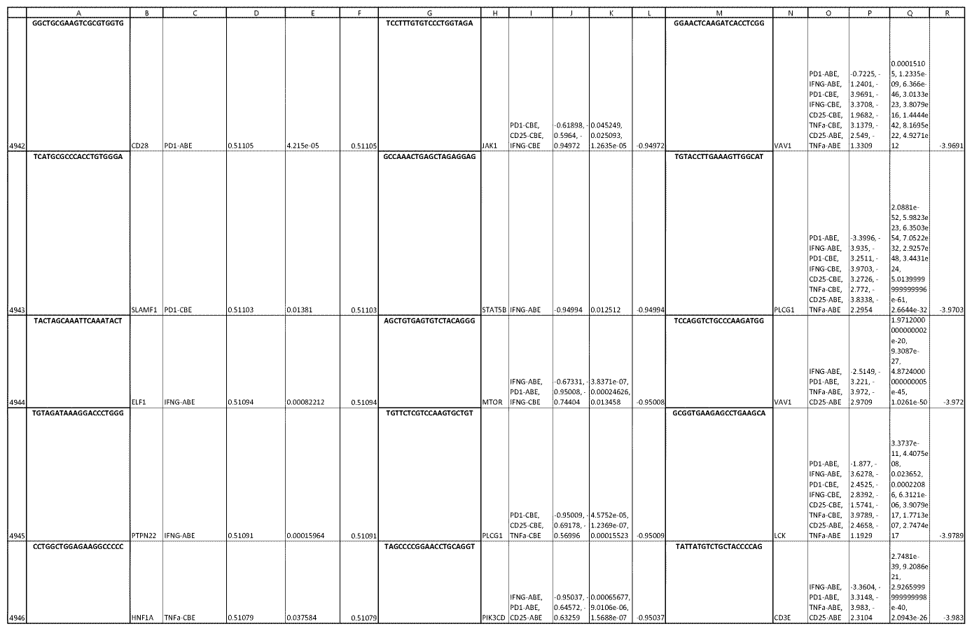

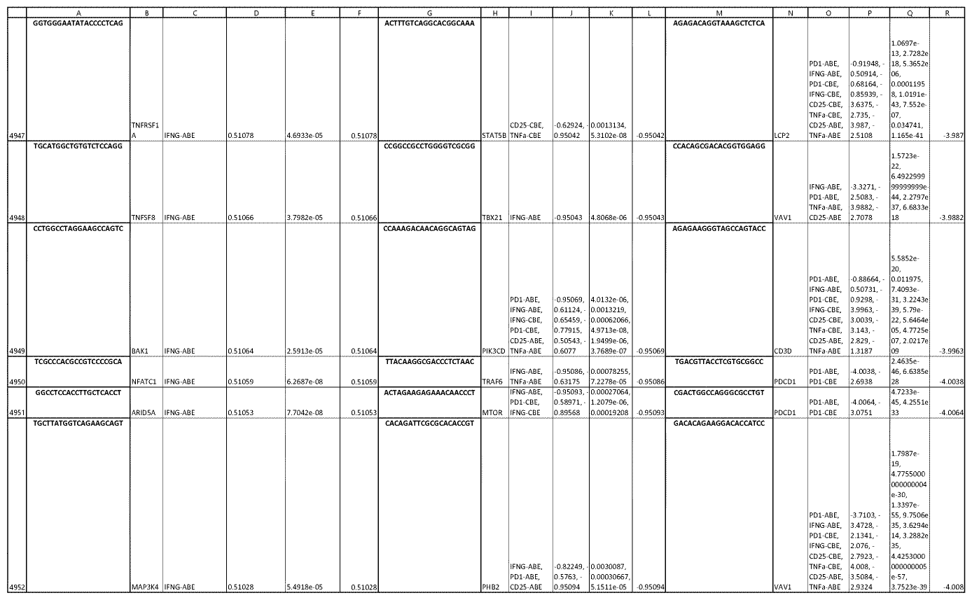

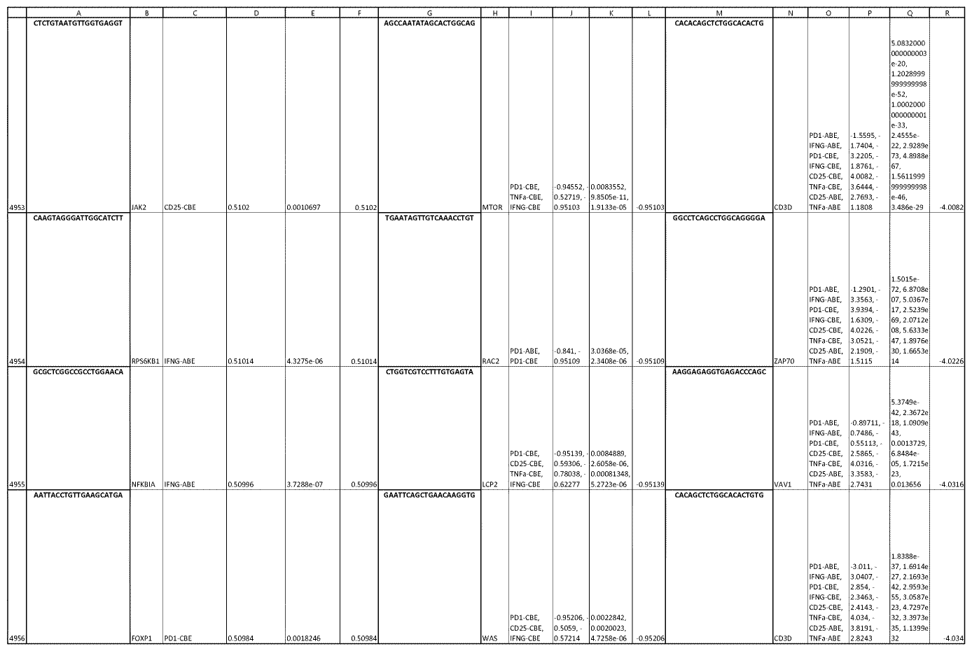

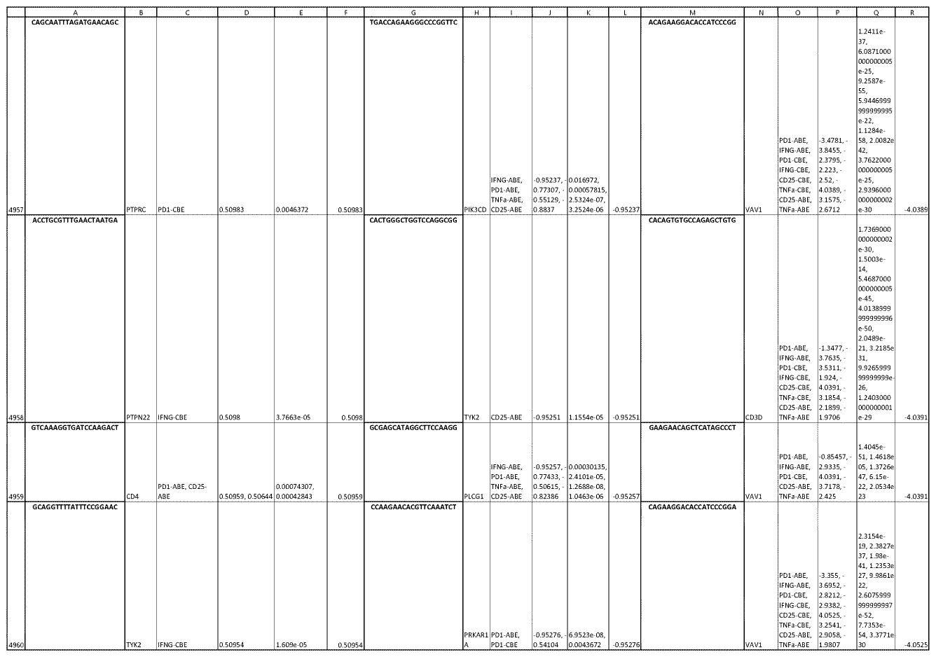

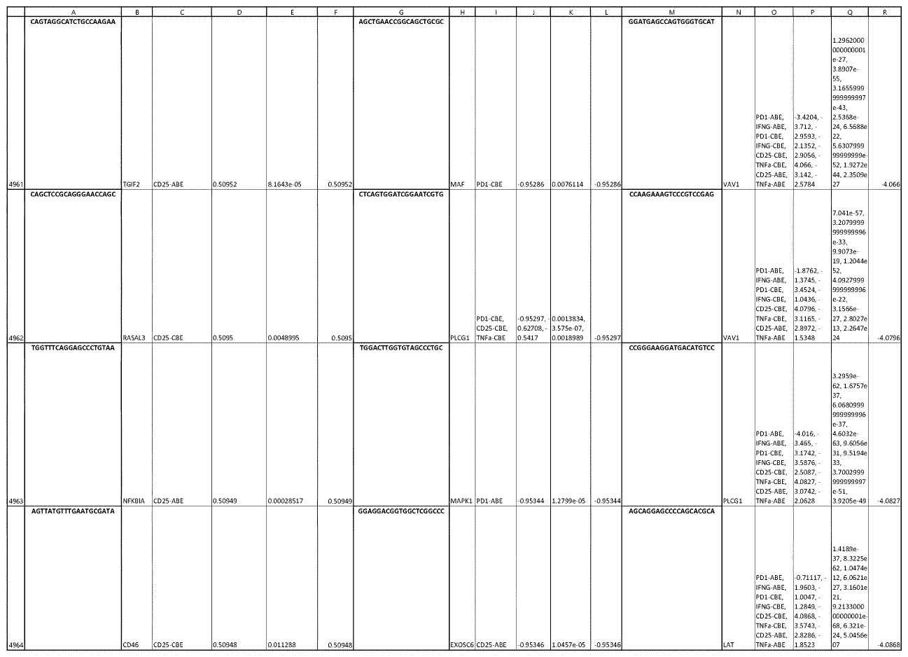

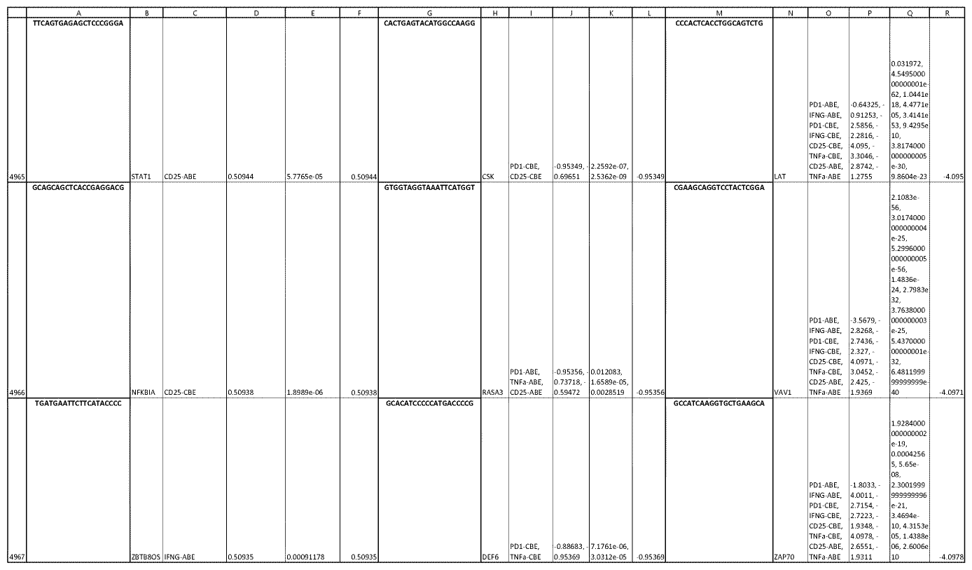

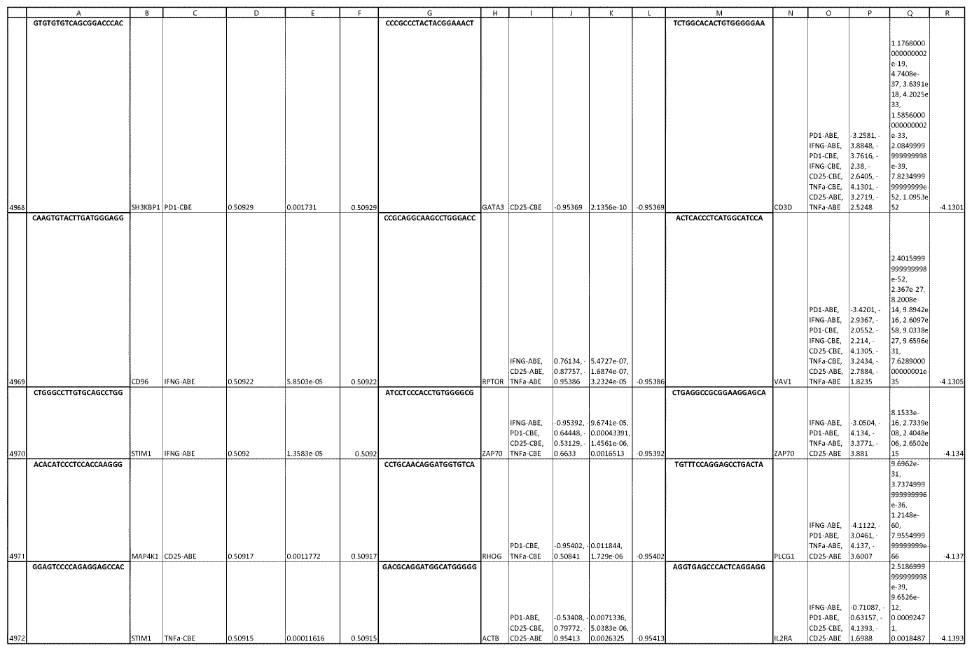

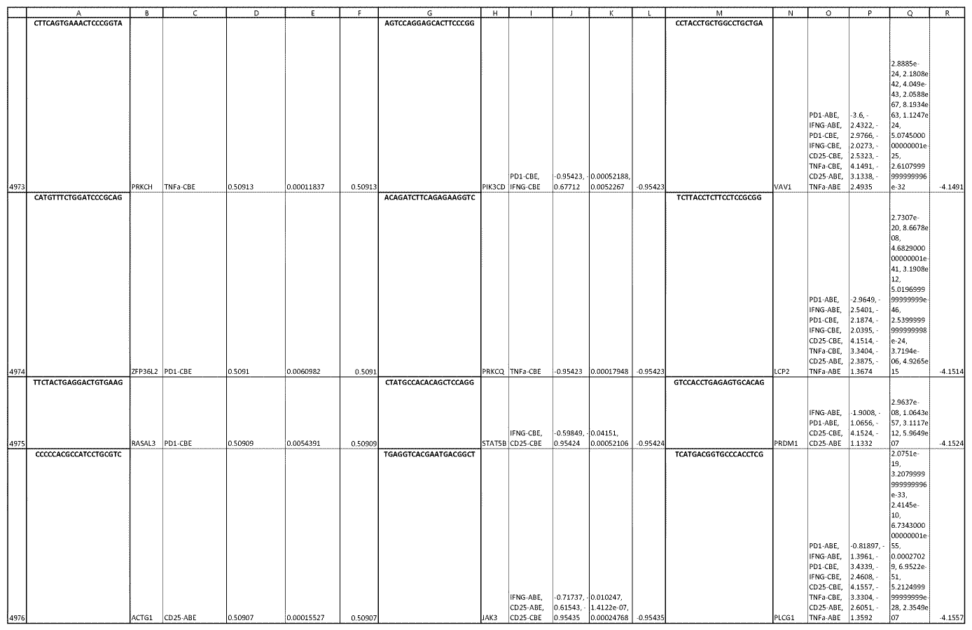

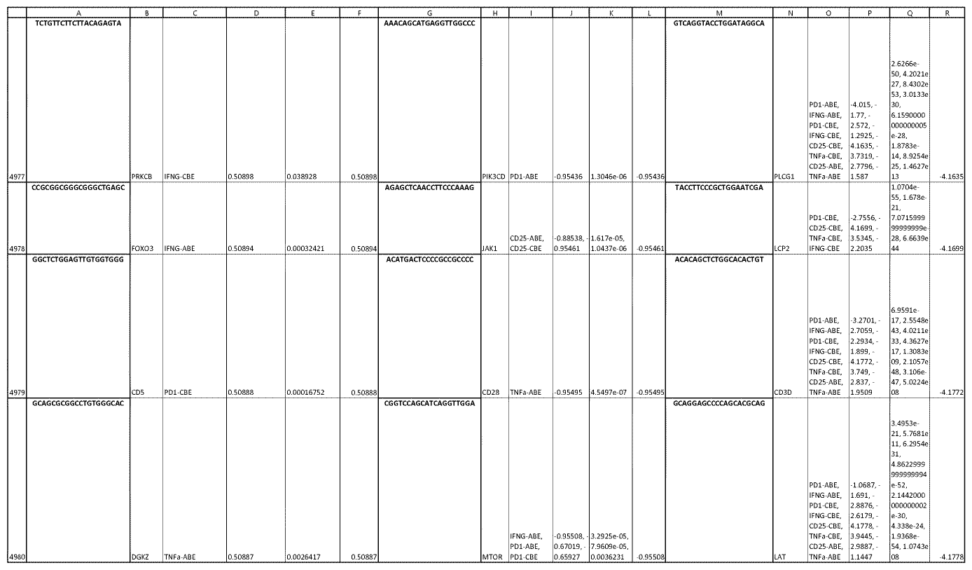

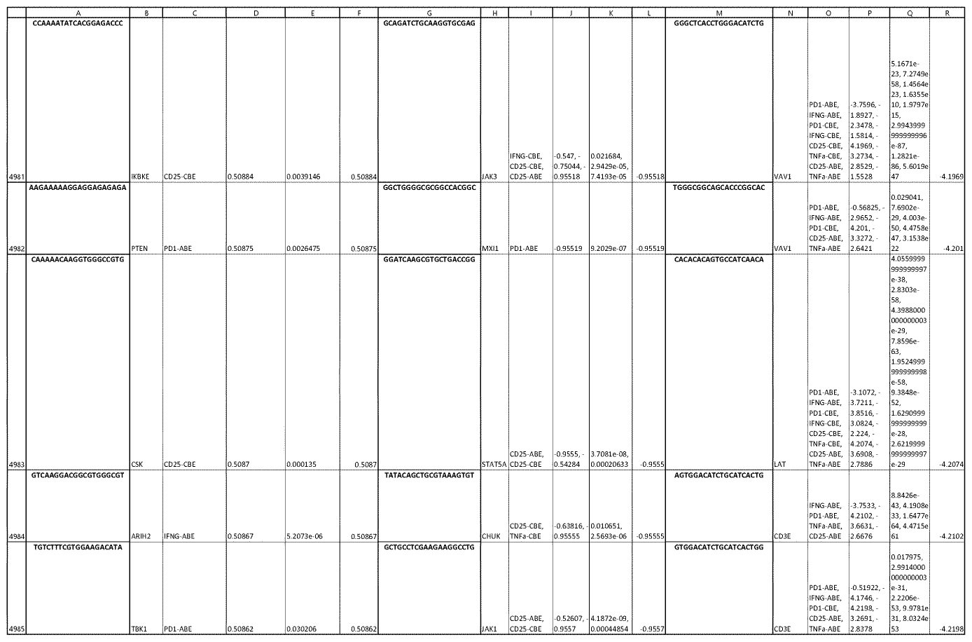

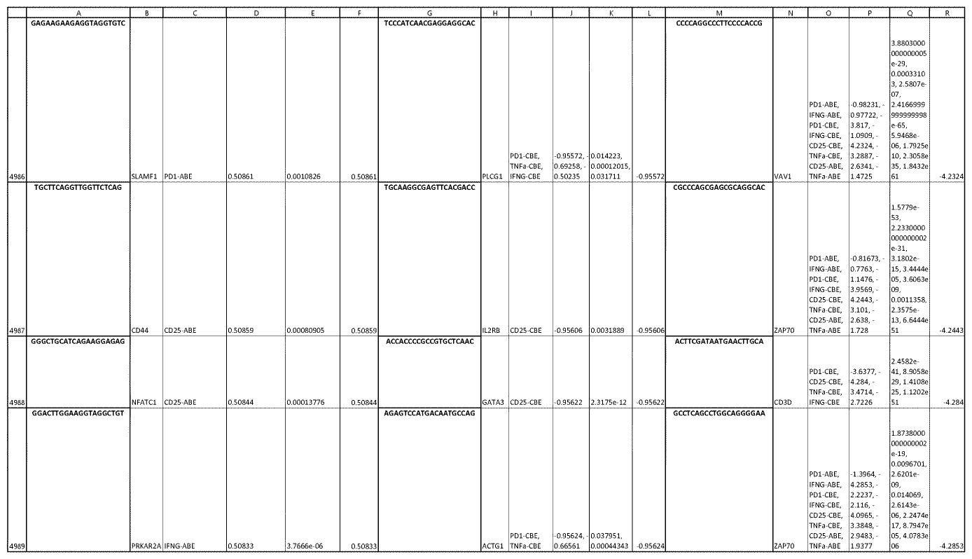

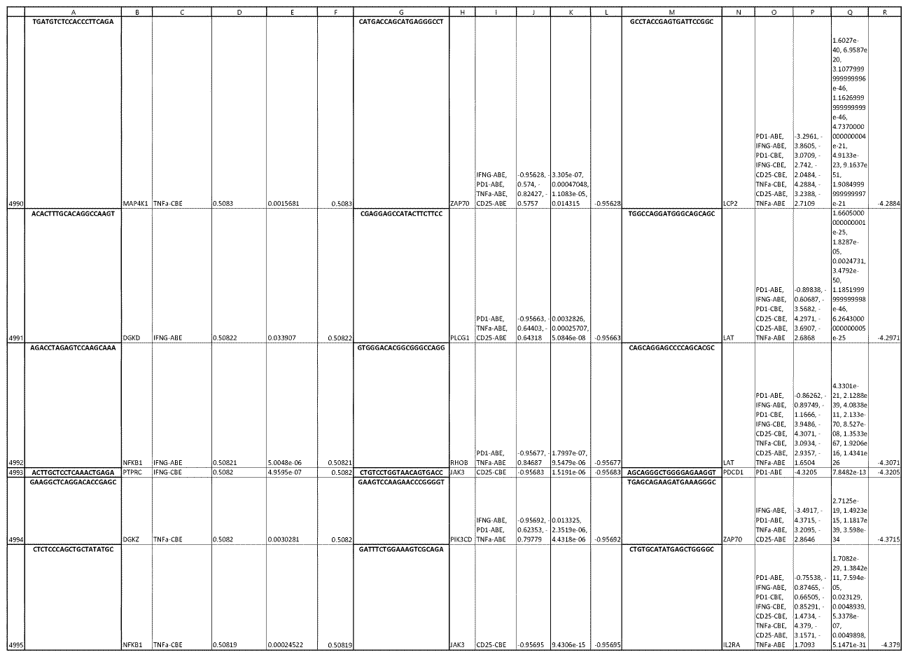

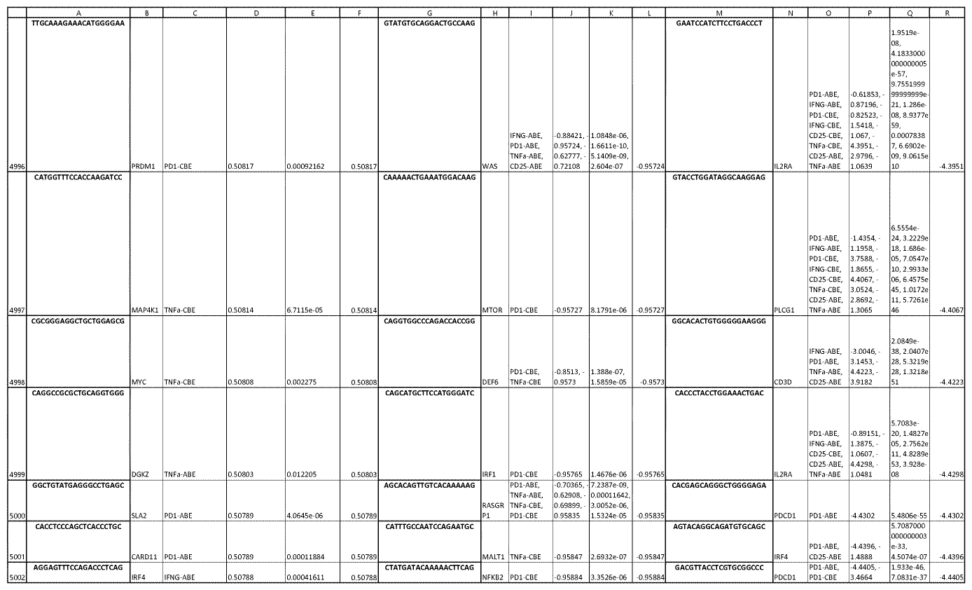

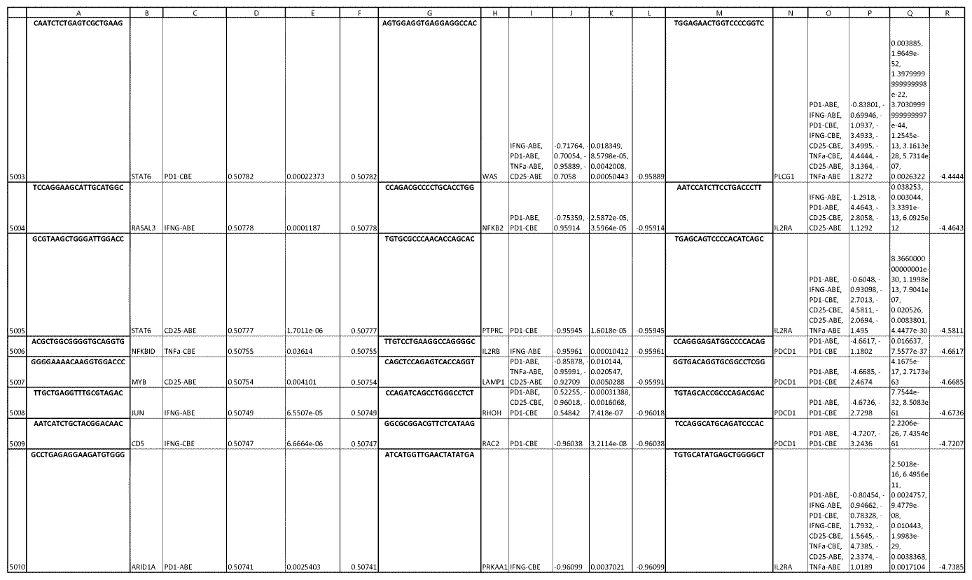

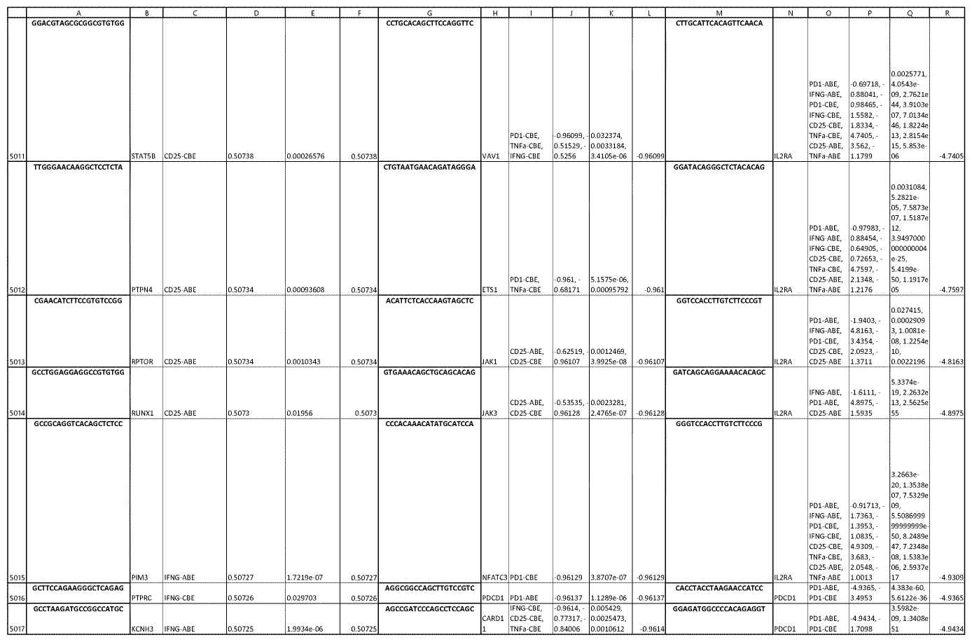

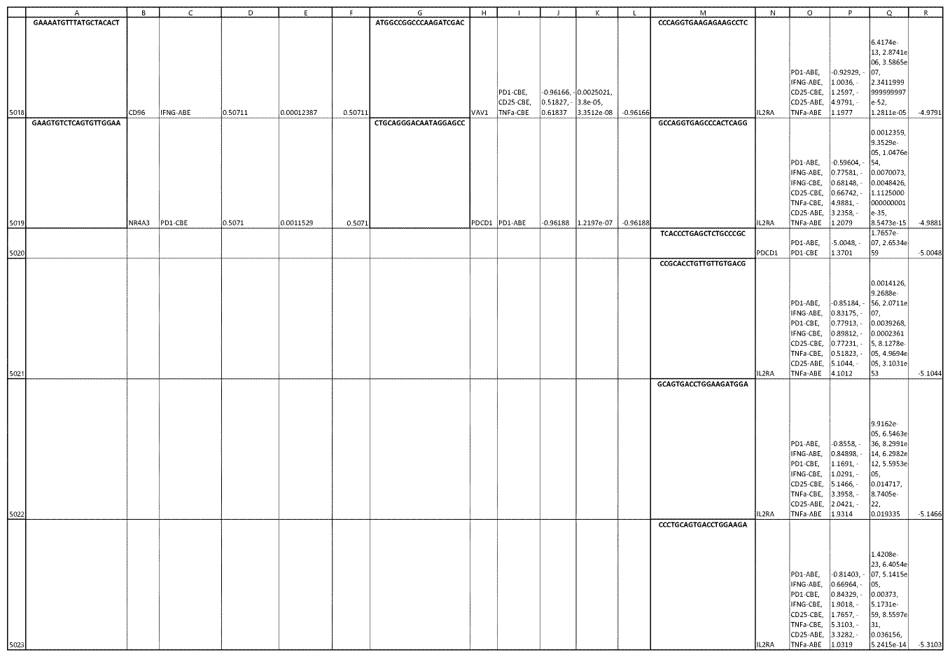

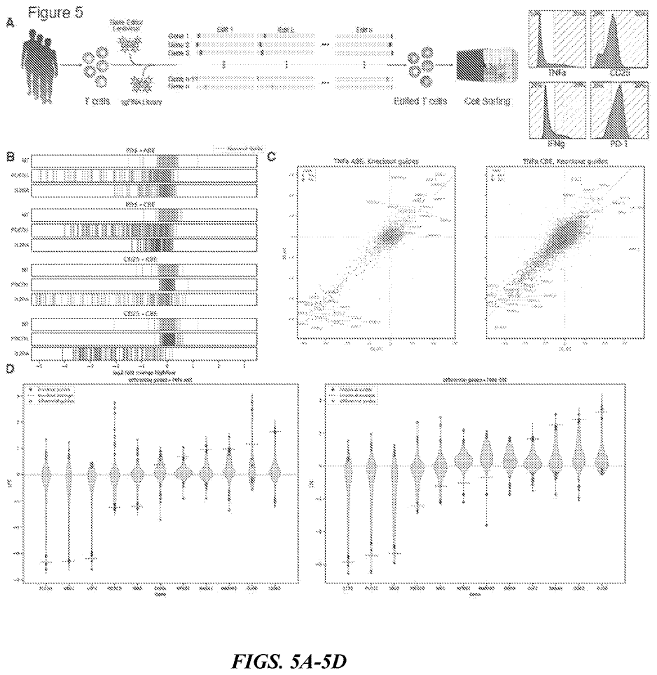

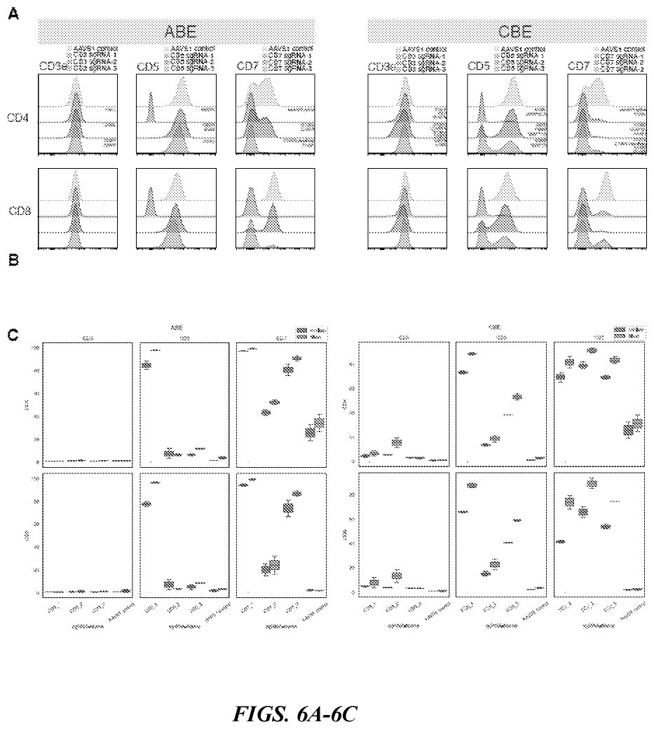

FIGS. 1-4 provide genes/sites/guides counts for decreasing stringency (maximum log 2 fold change for each guide (increasing cutoffs of 0.5, 1.0, 2.0, 3.0)). FIGS. 1A-4A have the genes that were modified and the modified sites and FIGS. 1B-4B have the guide sequences. For example, FIGS. 1A-1B list 36 genes, 1827 sites and 587 sgRNAs (cutoff 3.0); FIGS. 2A-2B list 98 genes, 4892 sites, and 1774 sgRNAs (cutoff 2.0); FIGS. 3A-3B list 231 genes, 15571 sites, and 5855 sgRNAs (cutoff 1.0); and FIGS. 4A-4B: list 375 genes, 40905 sites, and 15058 sgRNAs.

All publications, patent applications, patents and other references mentioned herein are expressly incorporated by reference in their entirety, to the same extent as if each were incorporated by reference individually. In case of conflict, the present specification, including definitions, will control.

The following statements provide a summary of some aspects of the inventive nucleic acids, proteins and methods described herein.

### Statements:

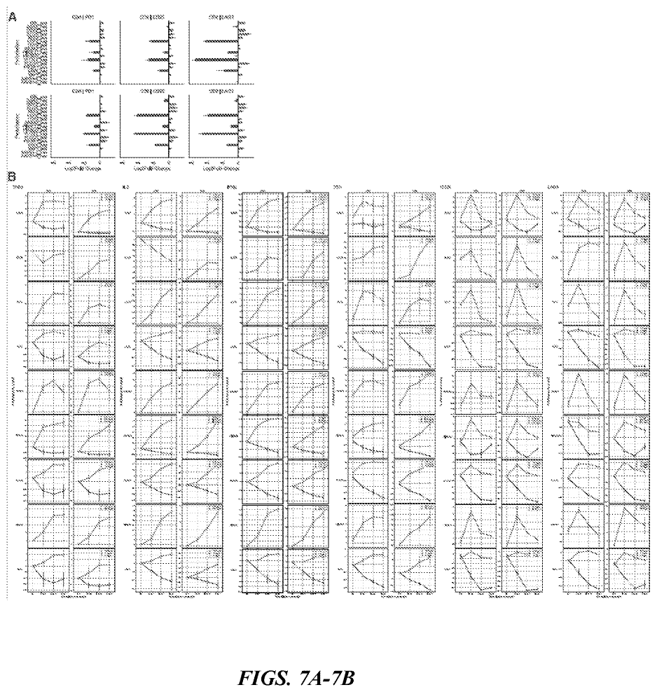

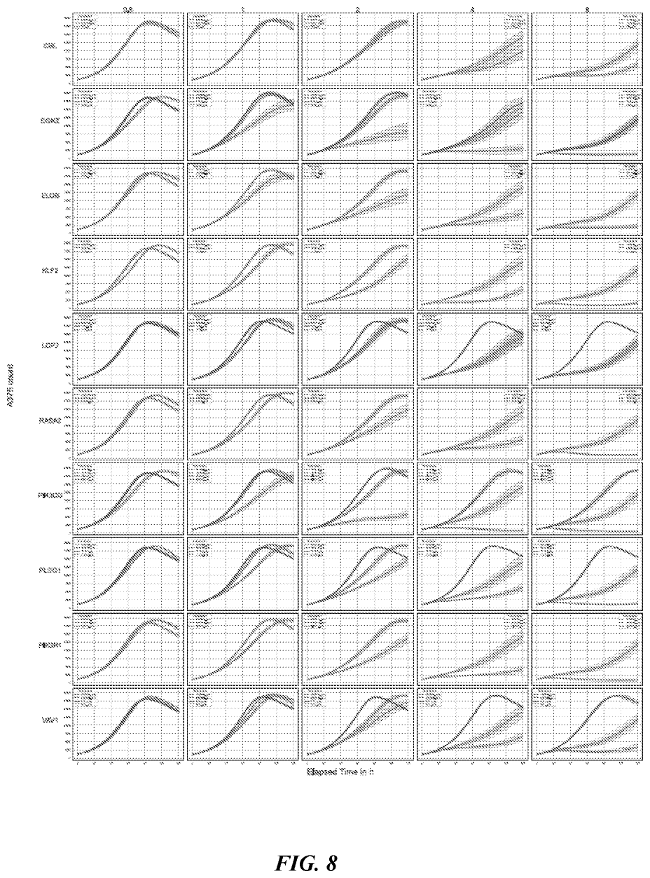

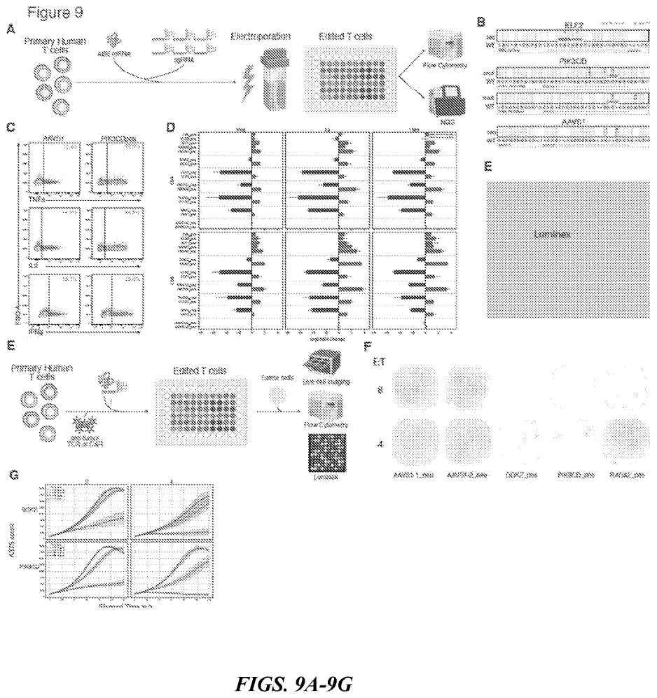

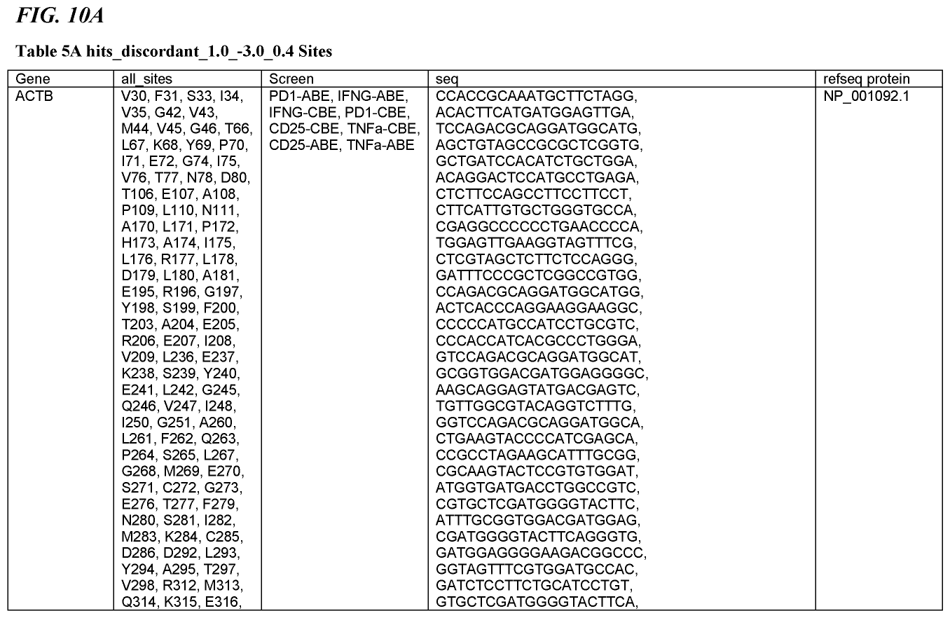

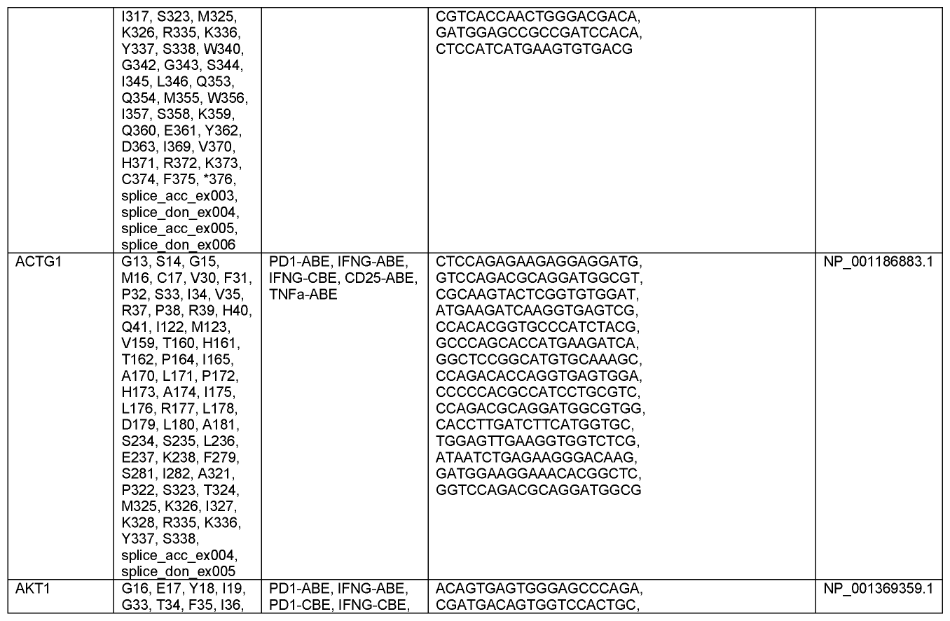

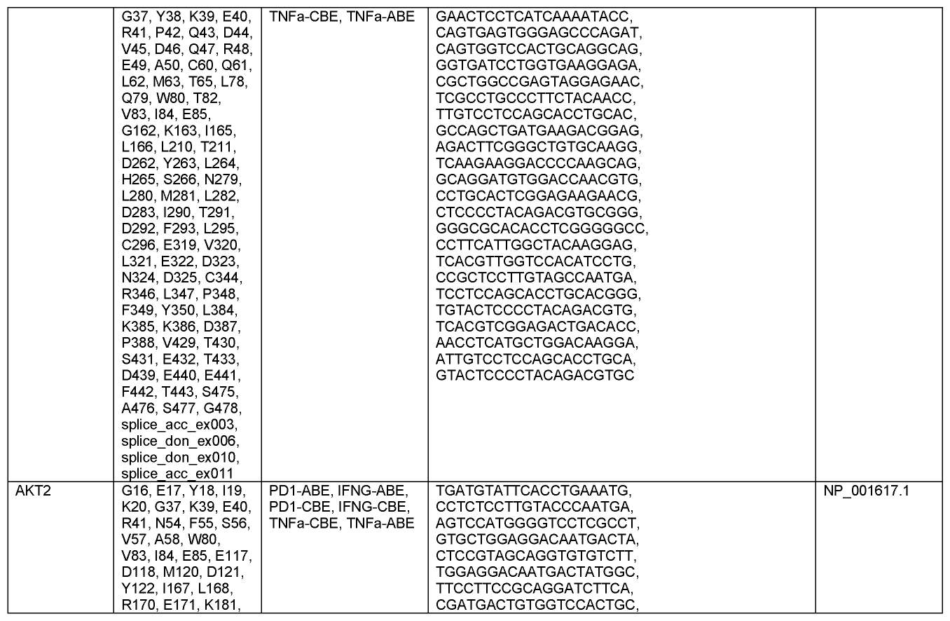

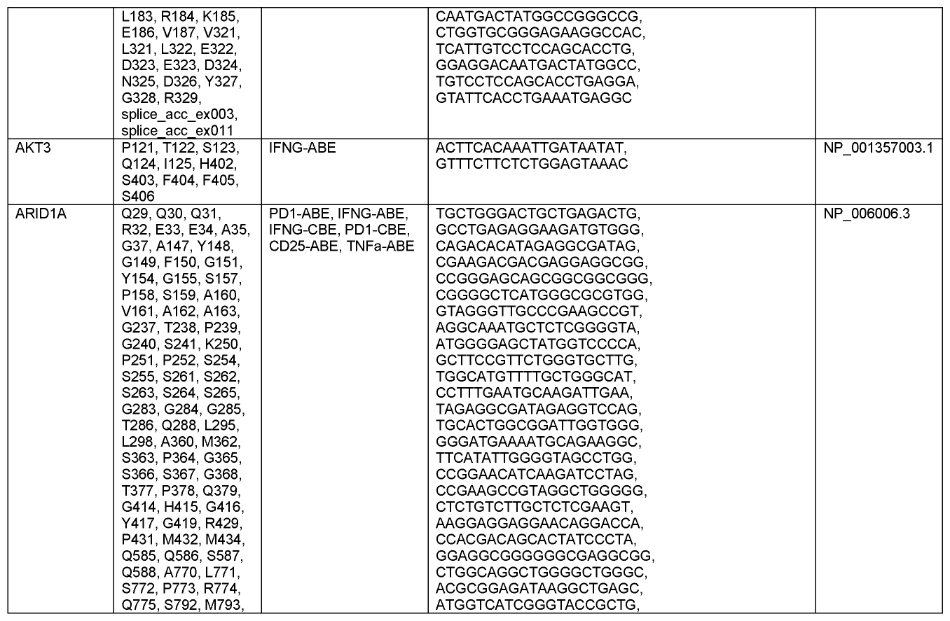

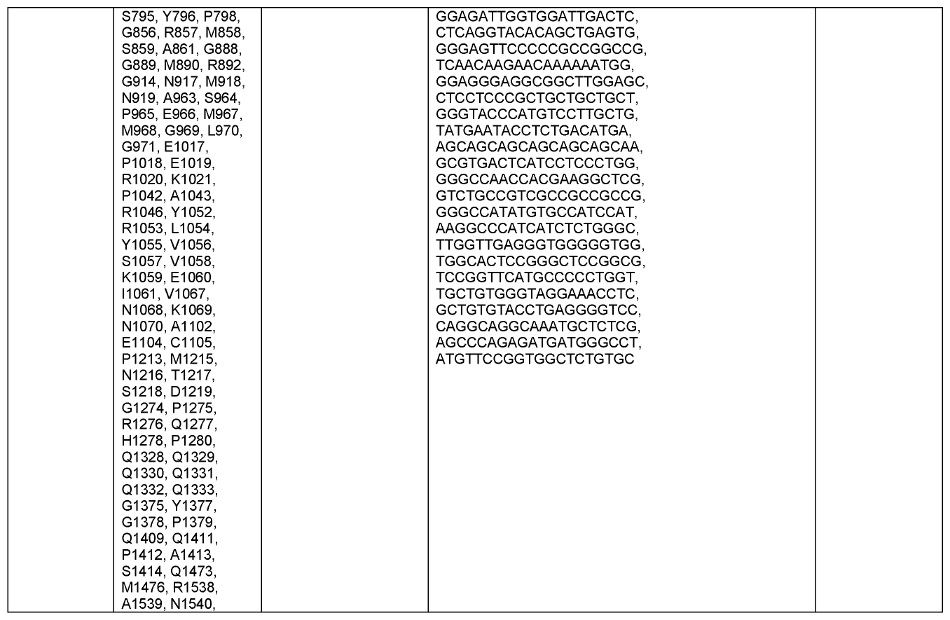

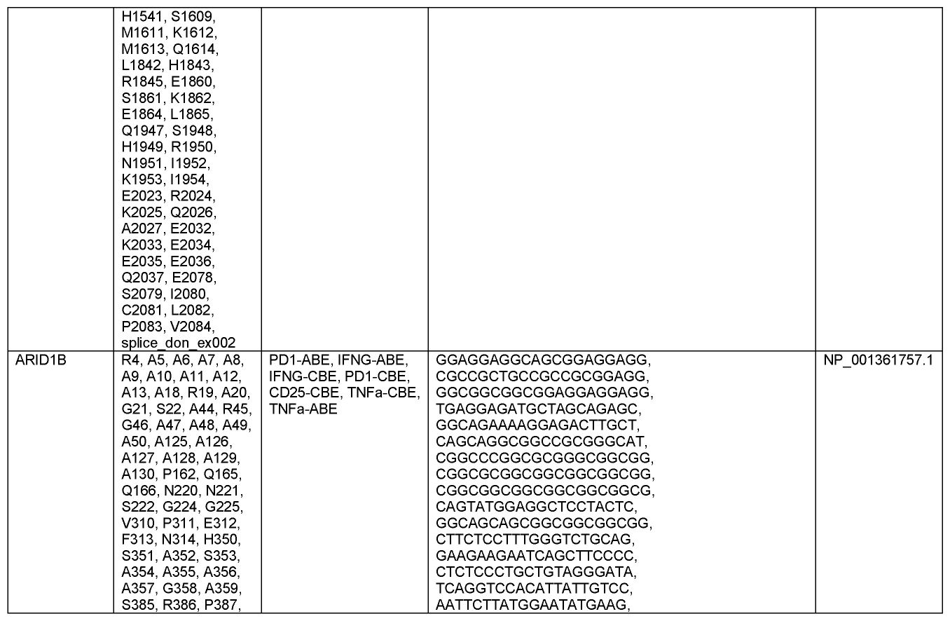

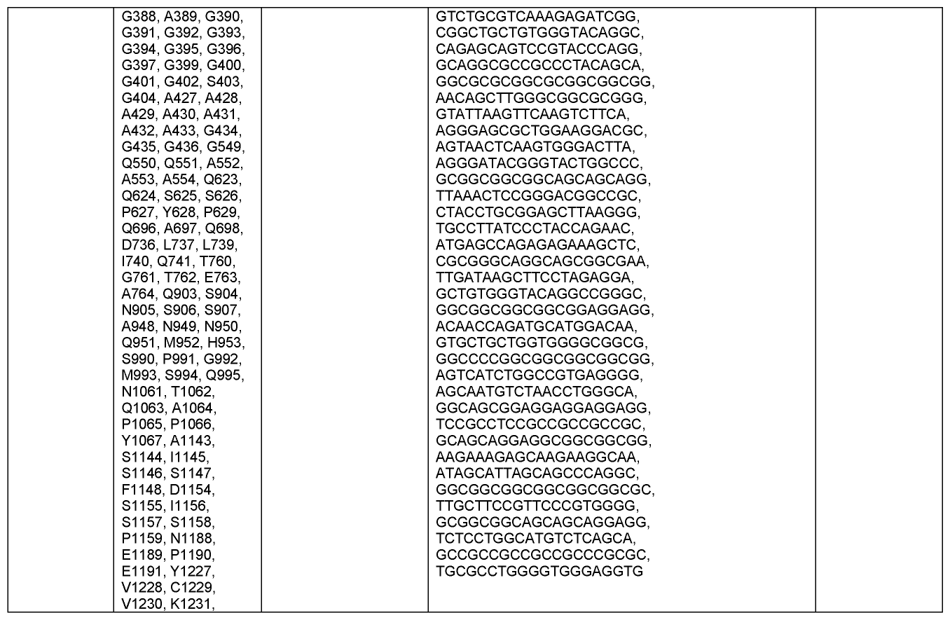

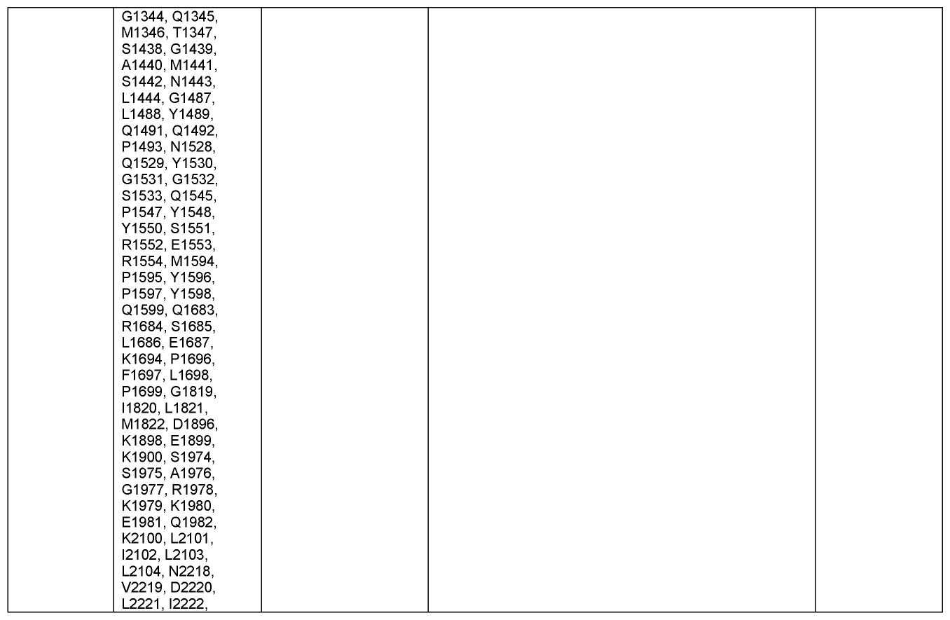

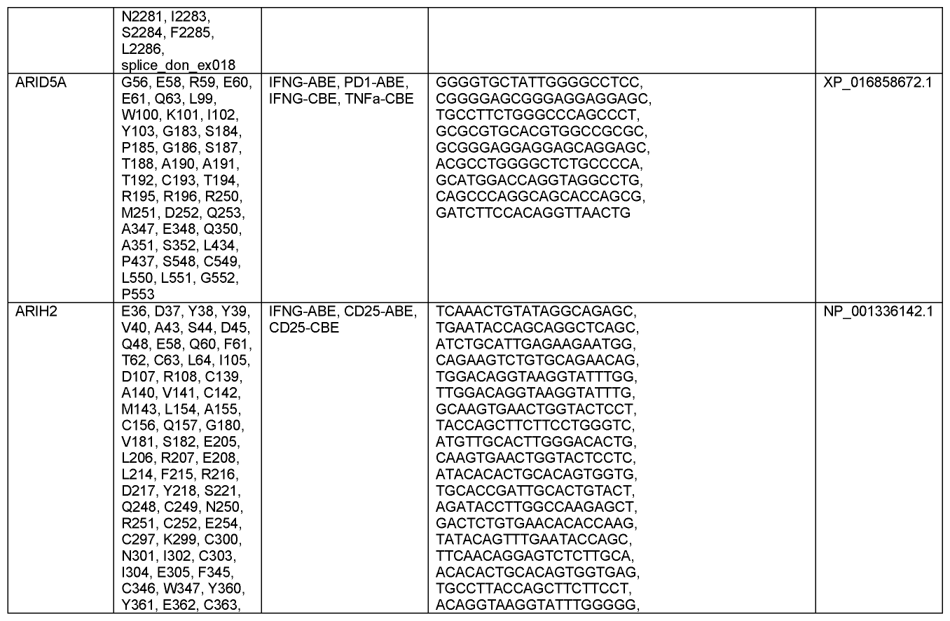

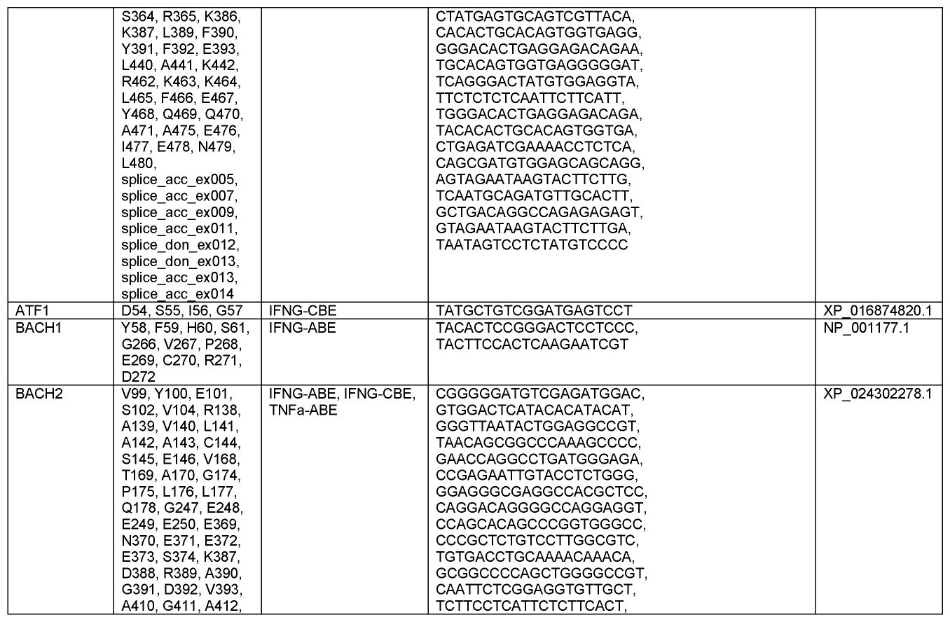

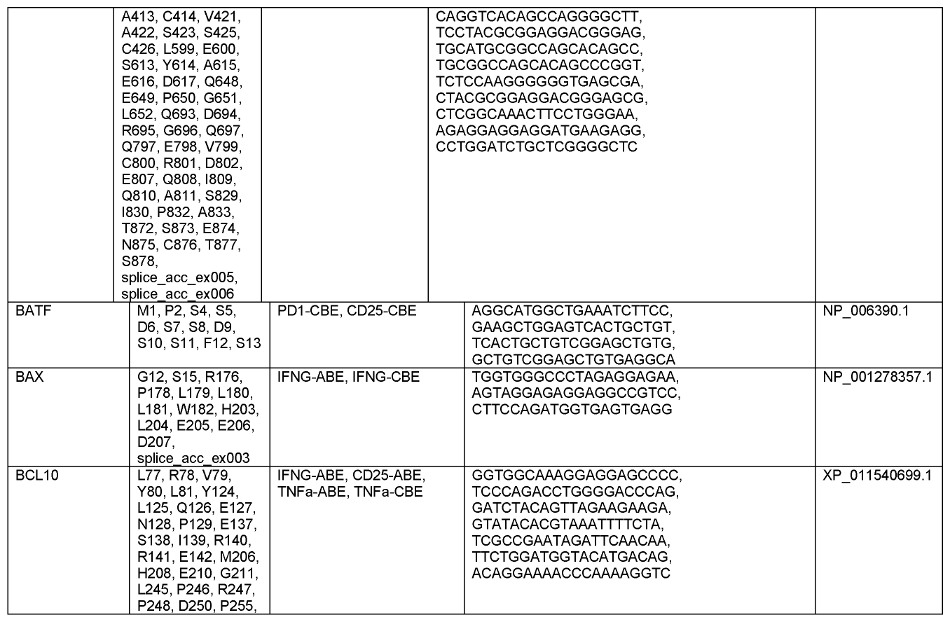

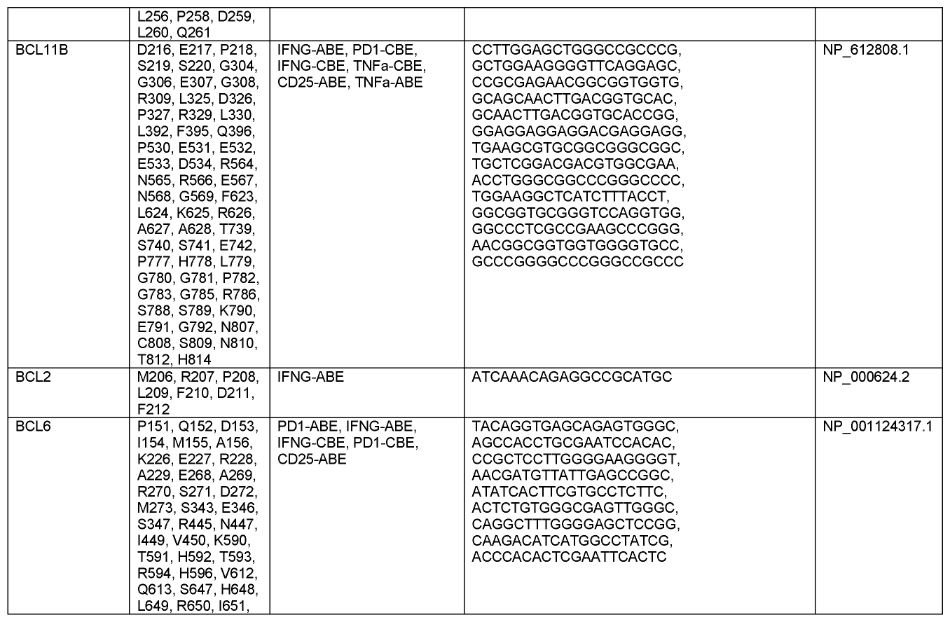

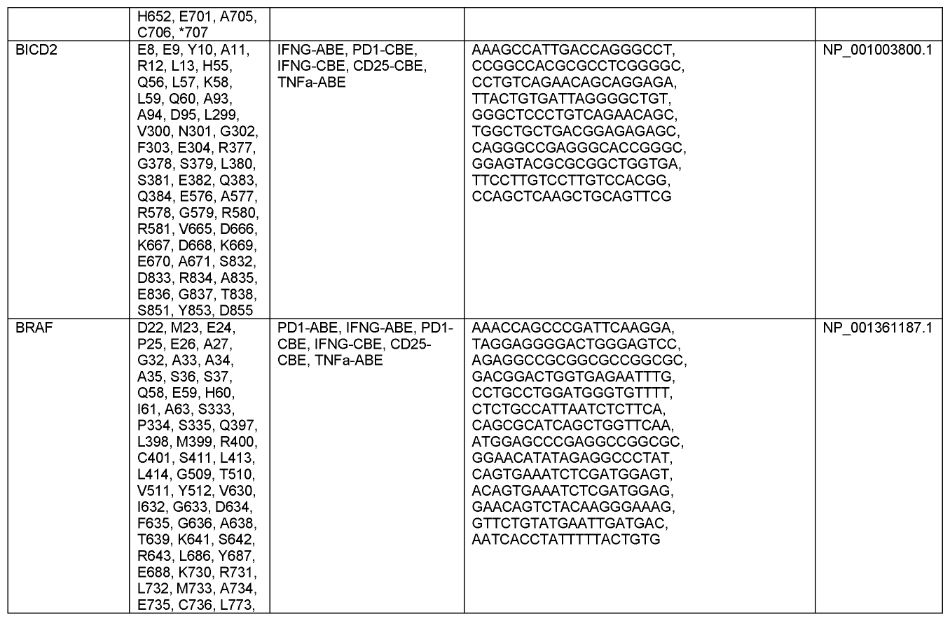

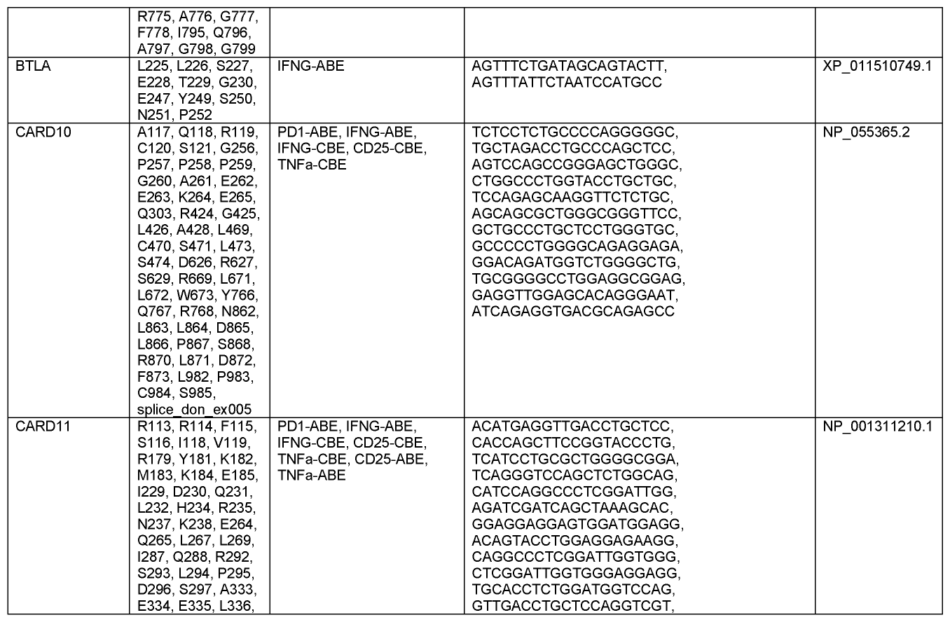

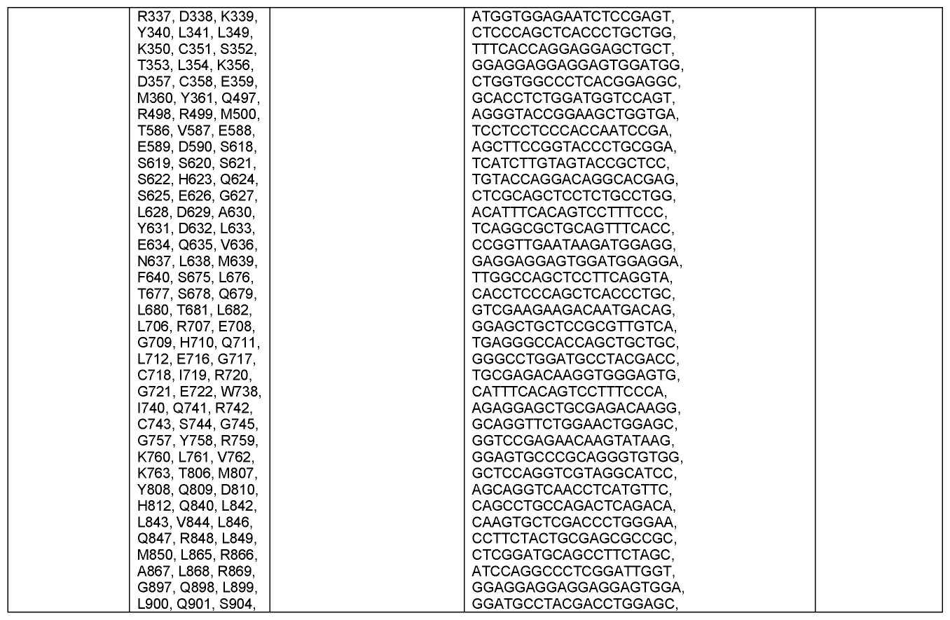

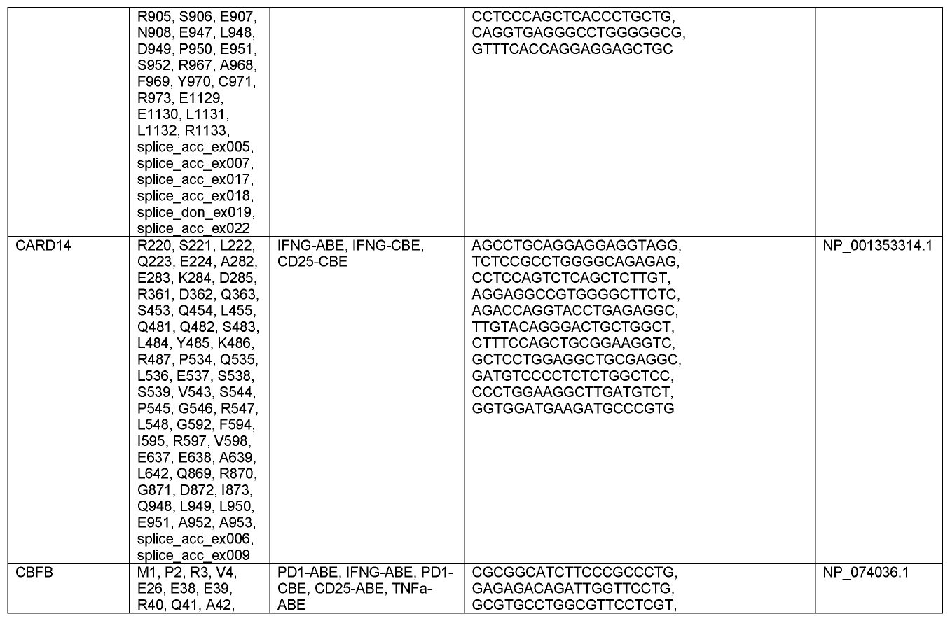

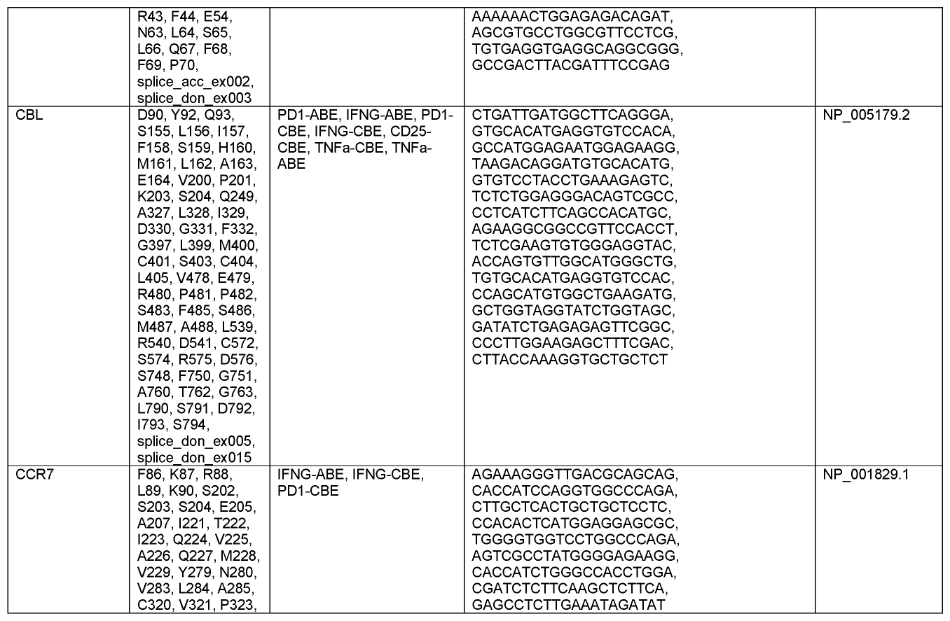

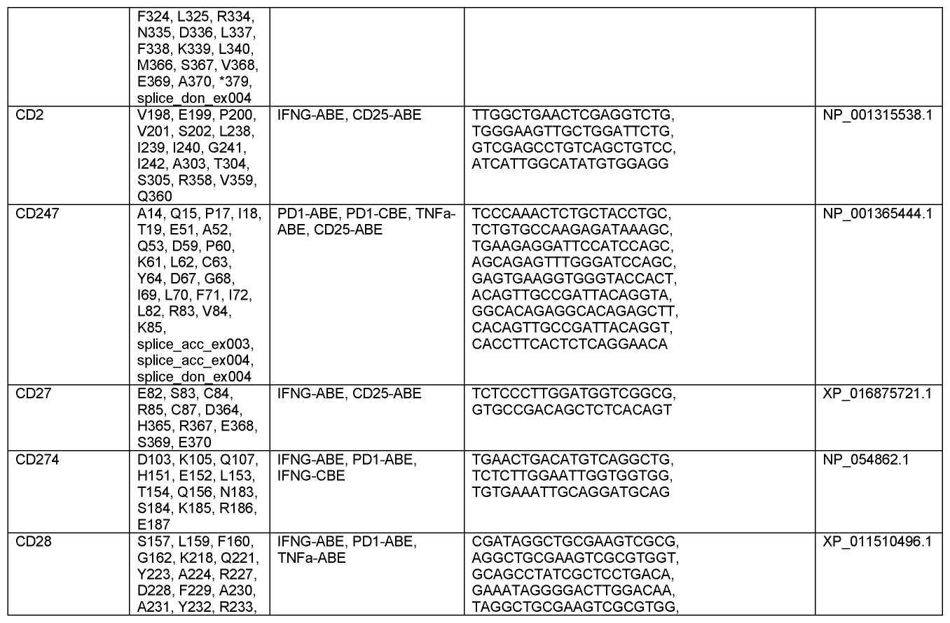

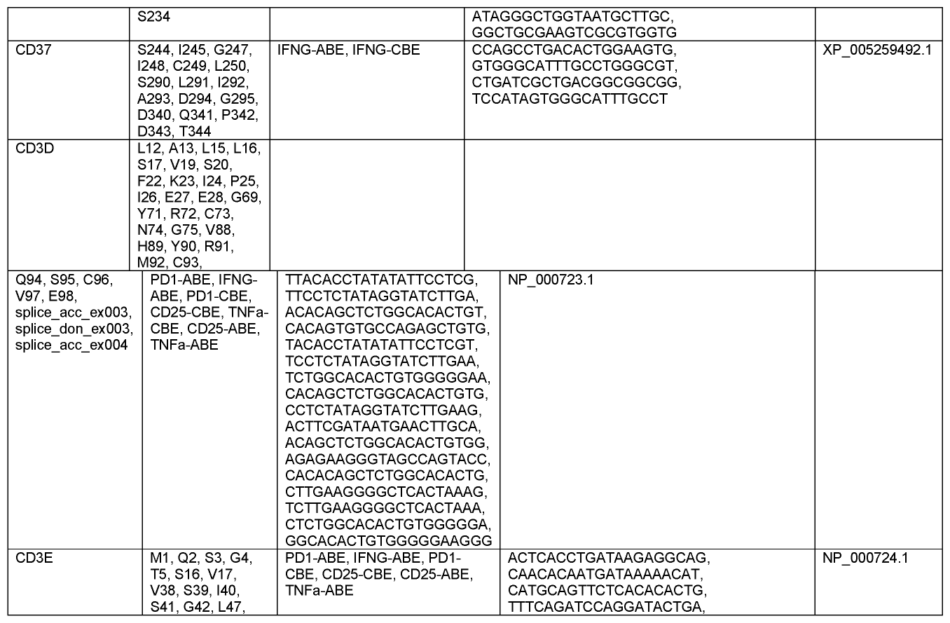

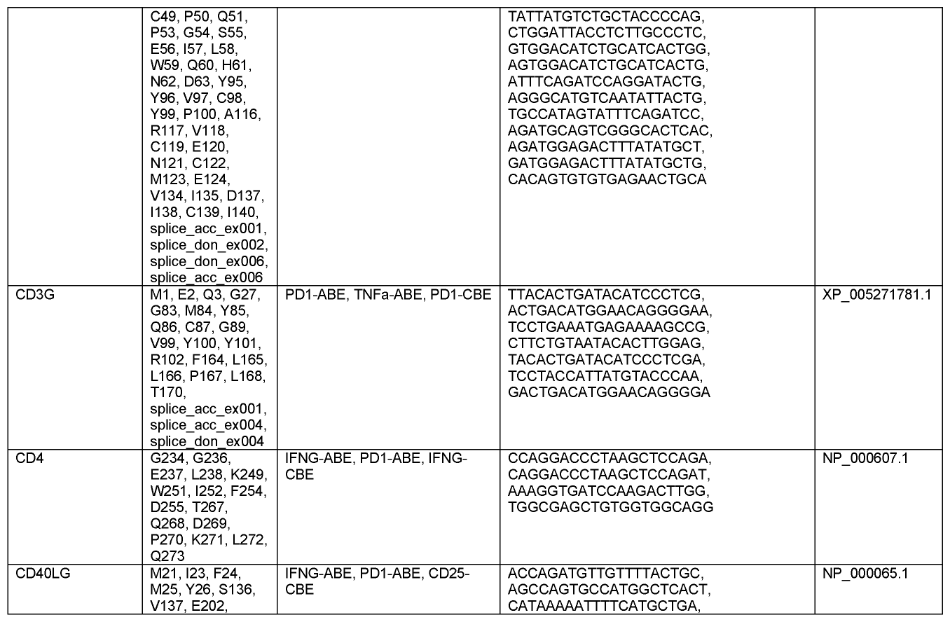

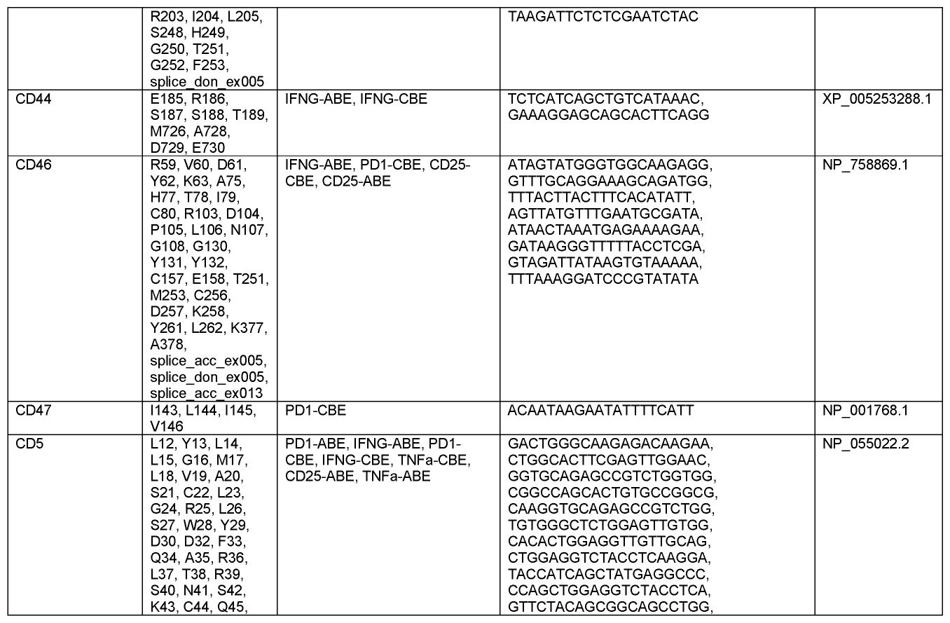

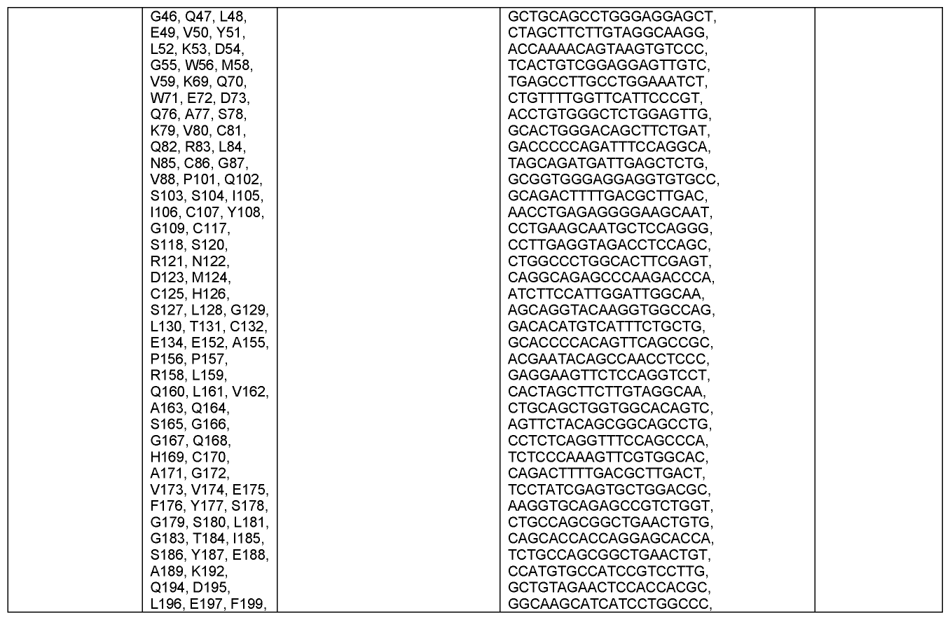

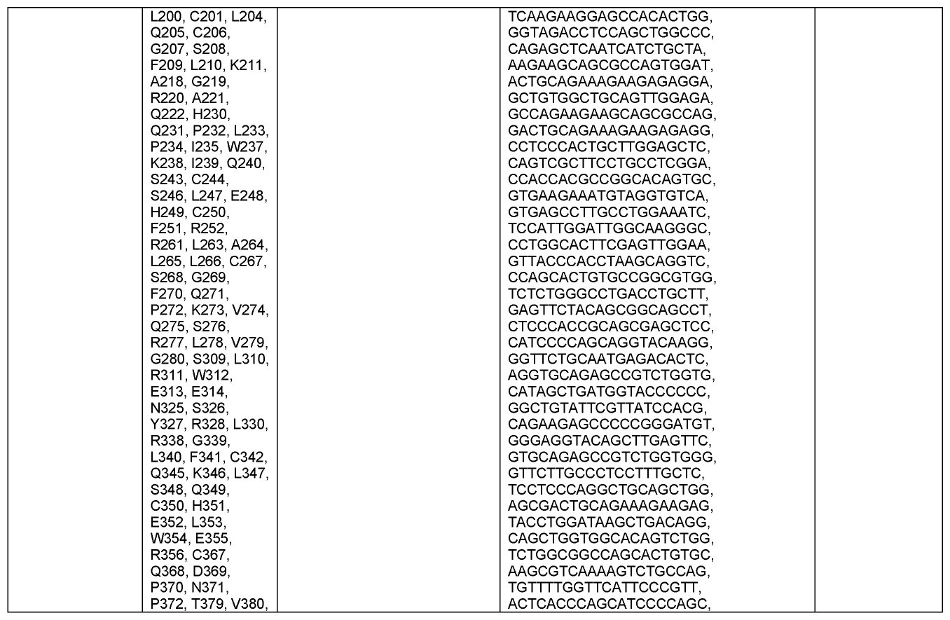

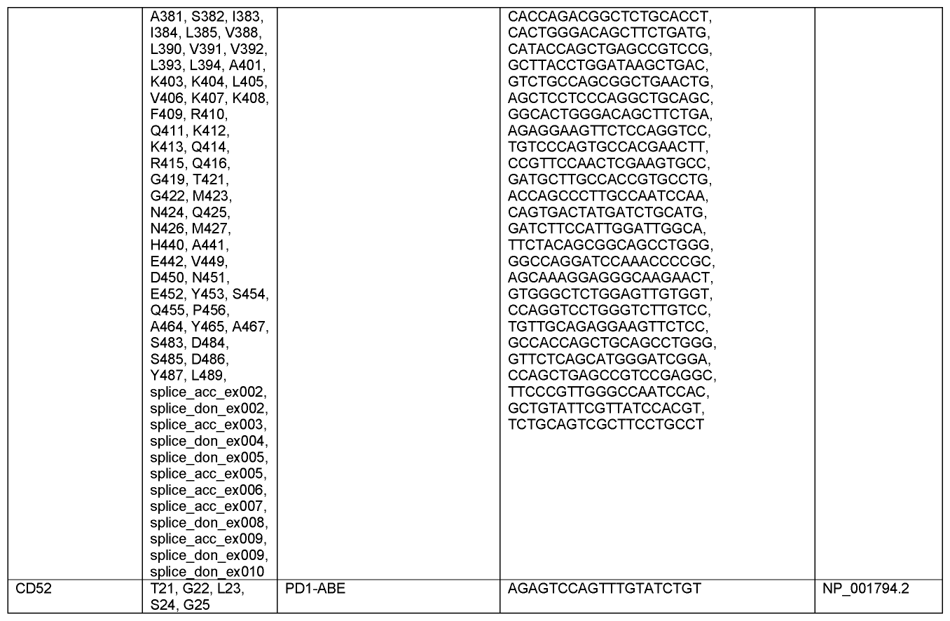

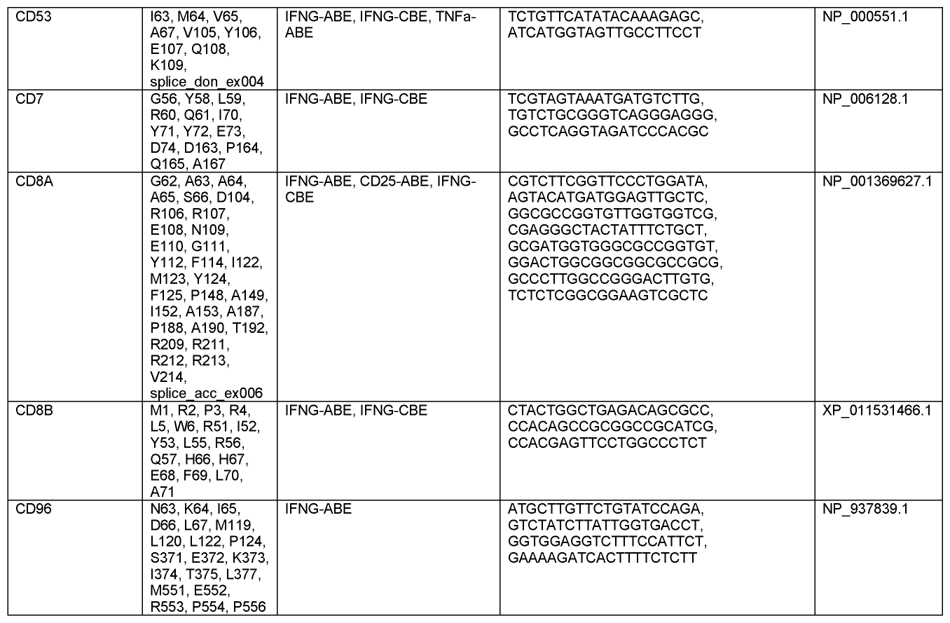

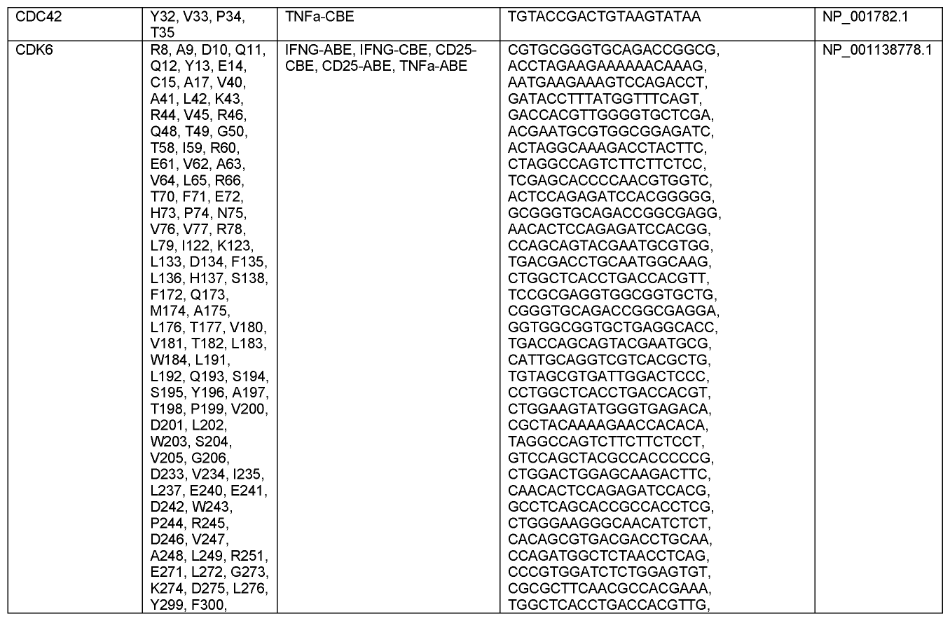

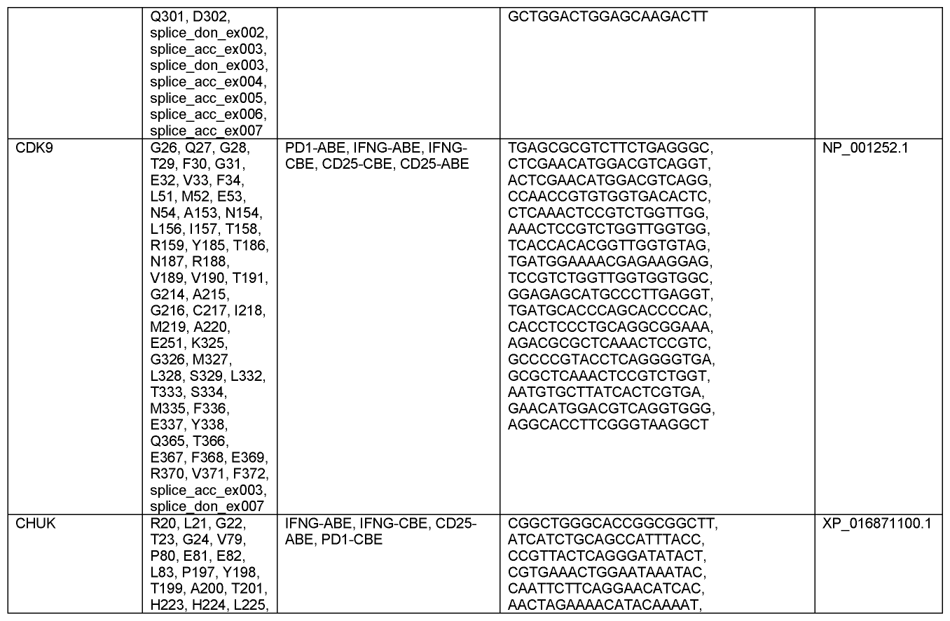

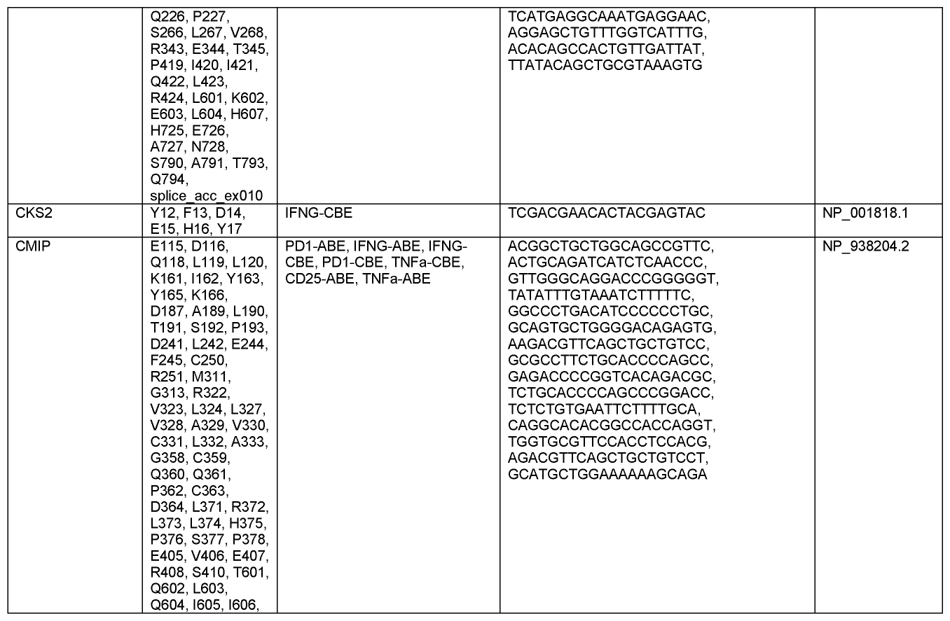

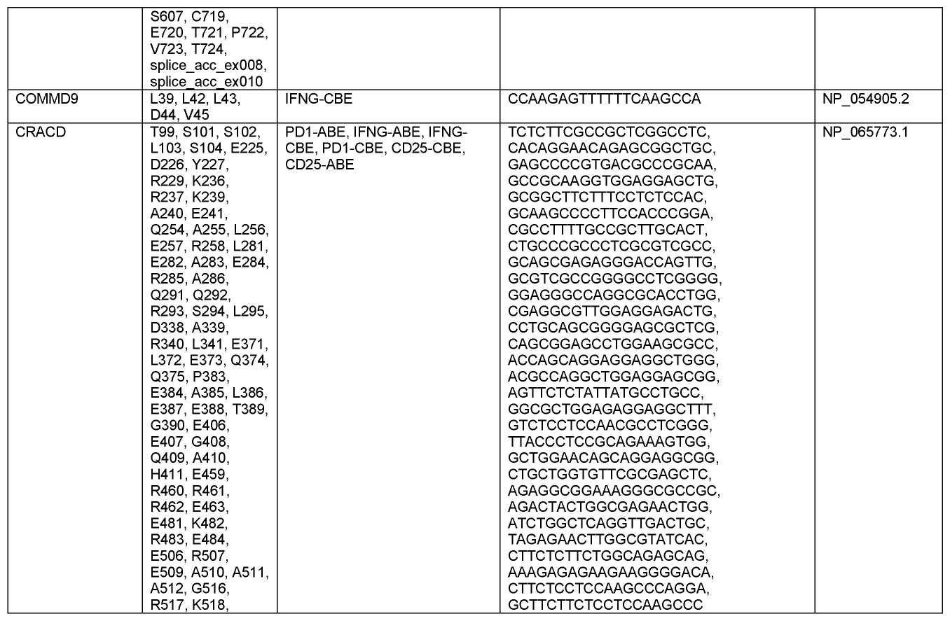

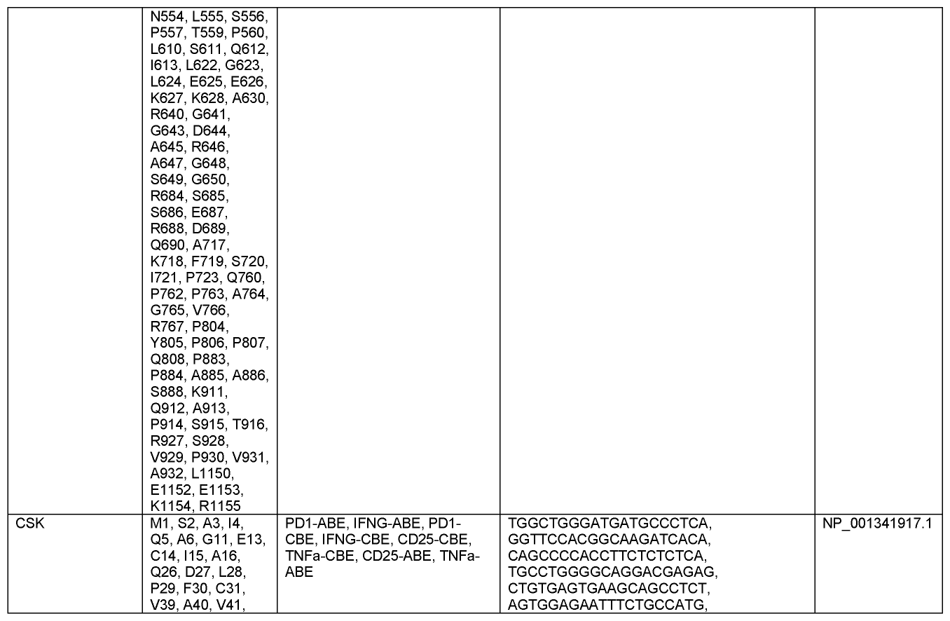

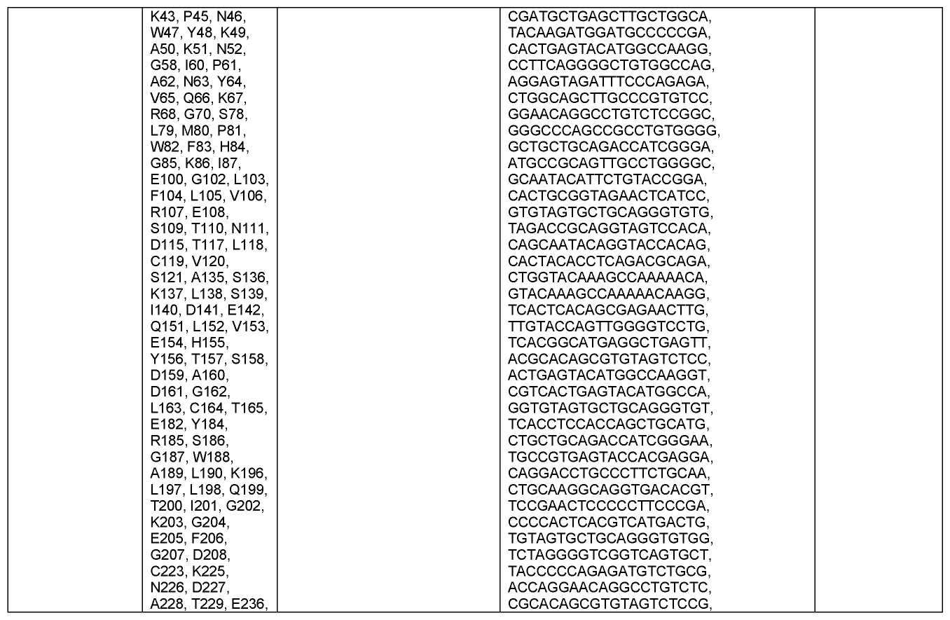

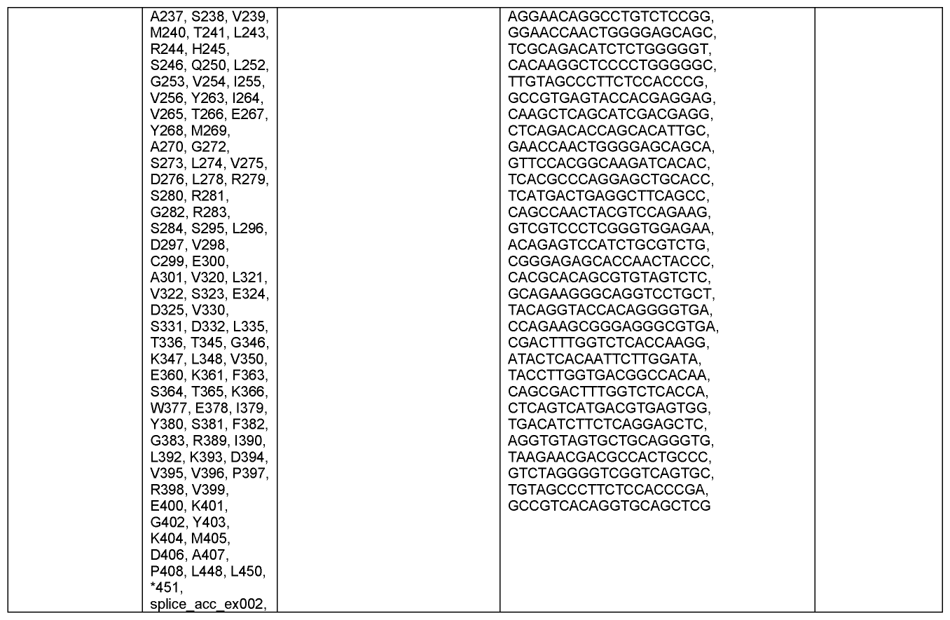

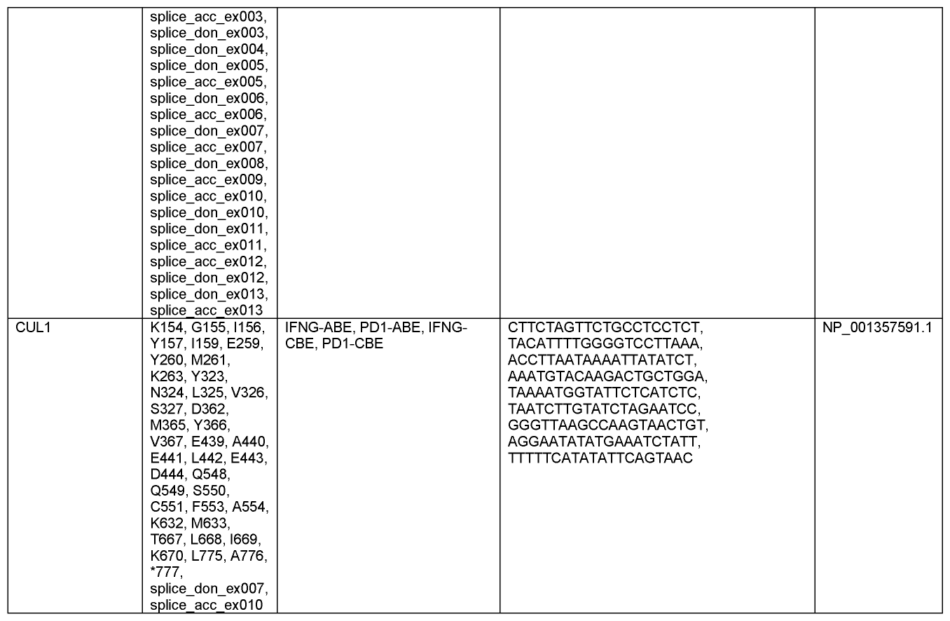

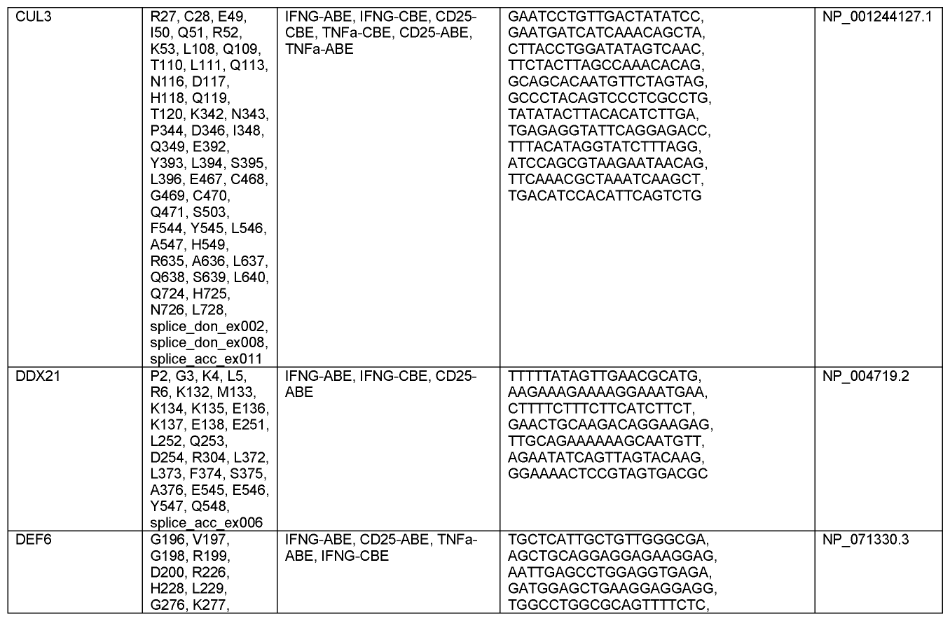

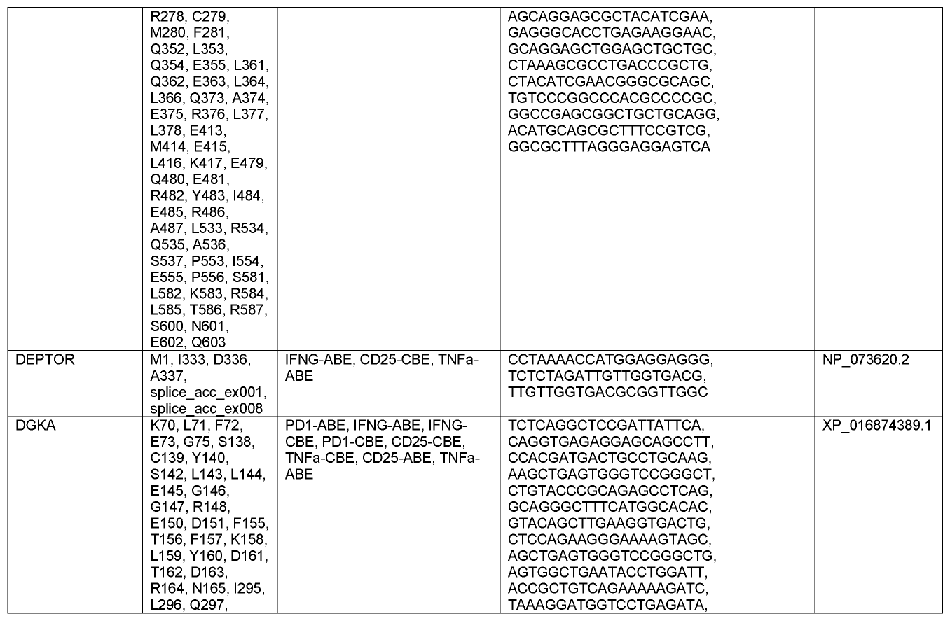

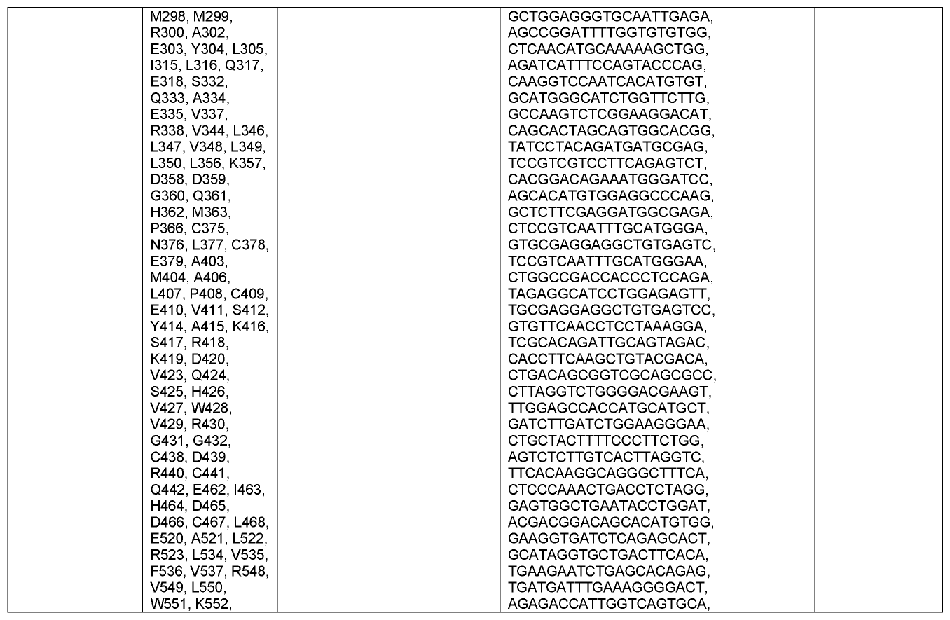

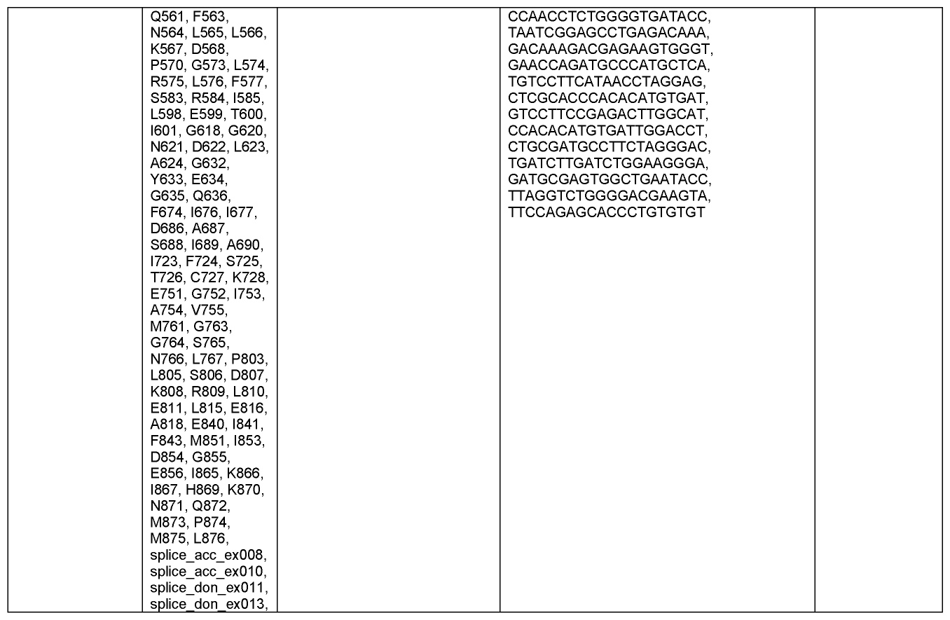

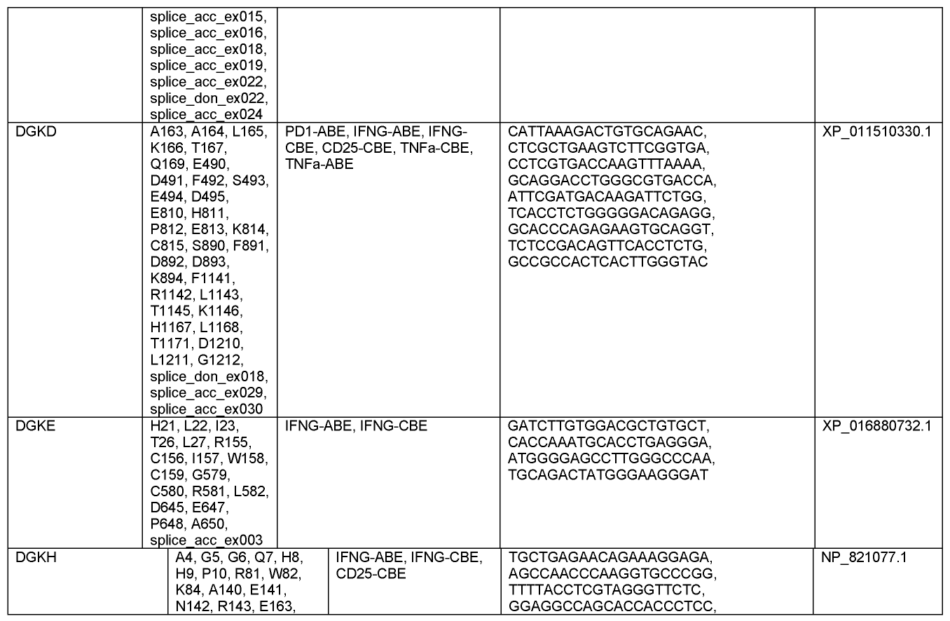

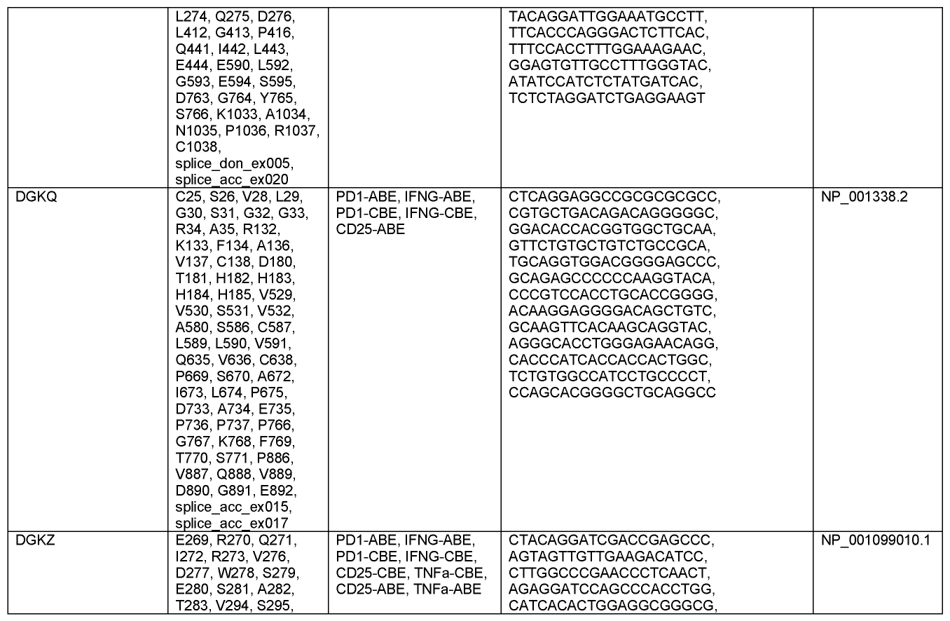

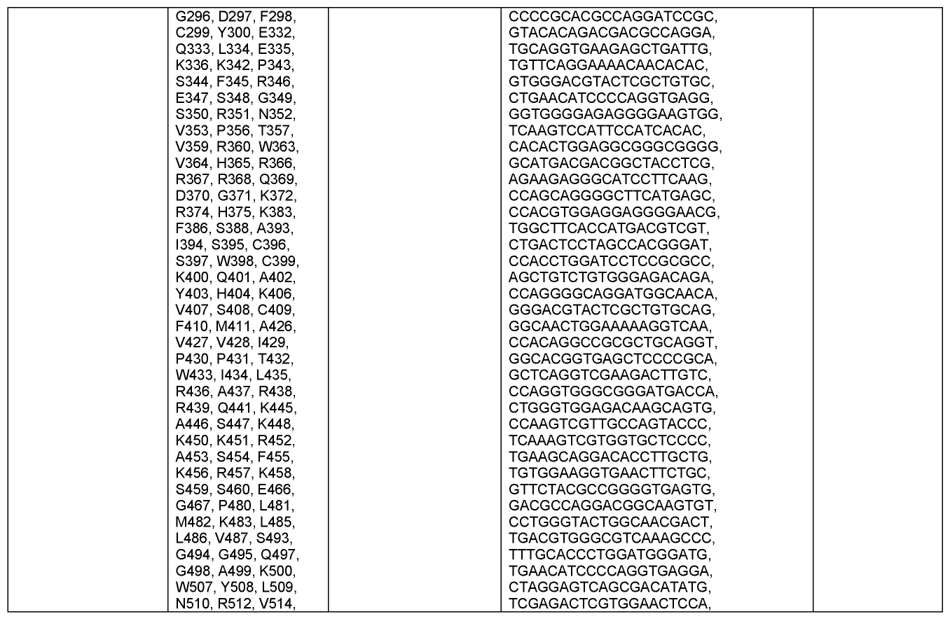

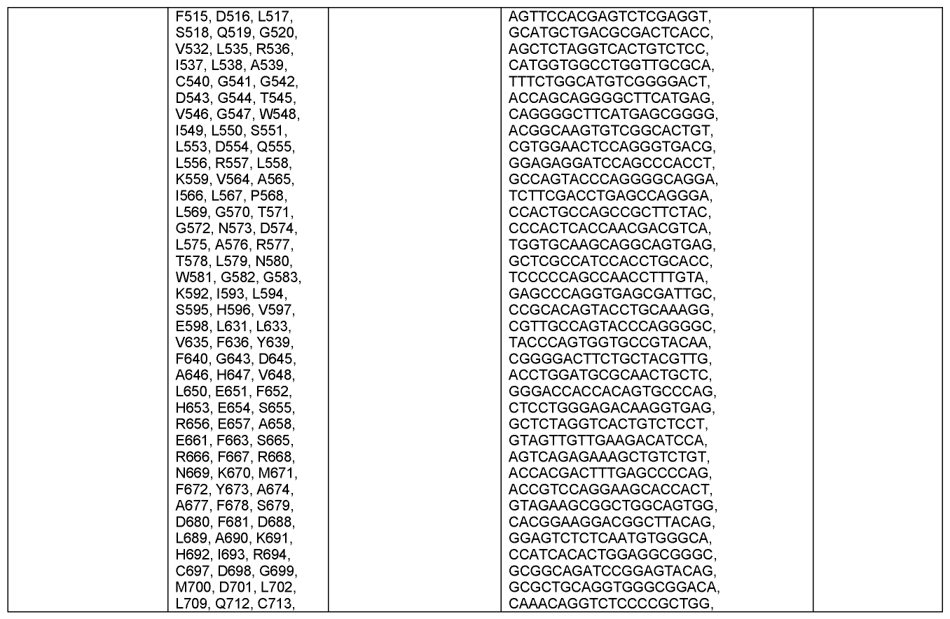

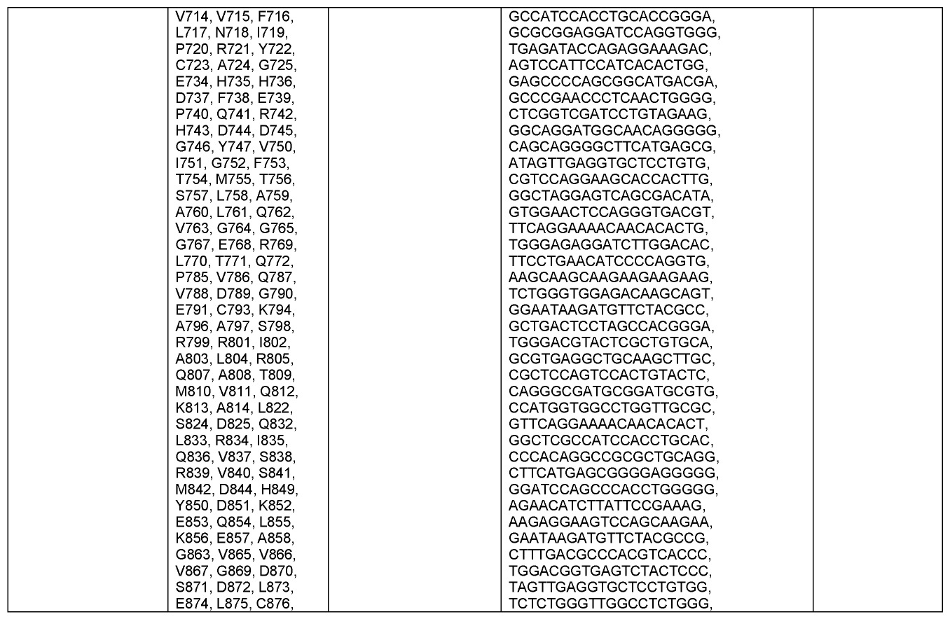

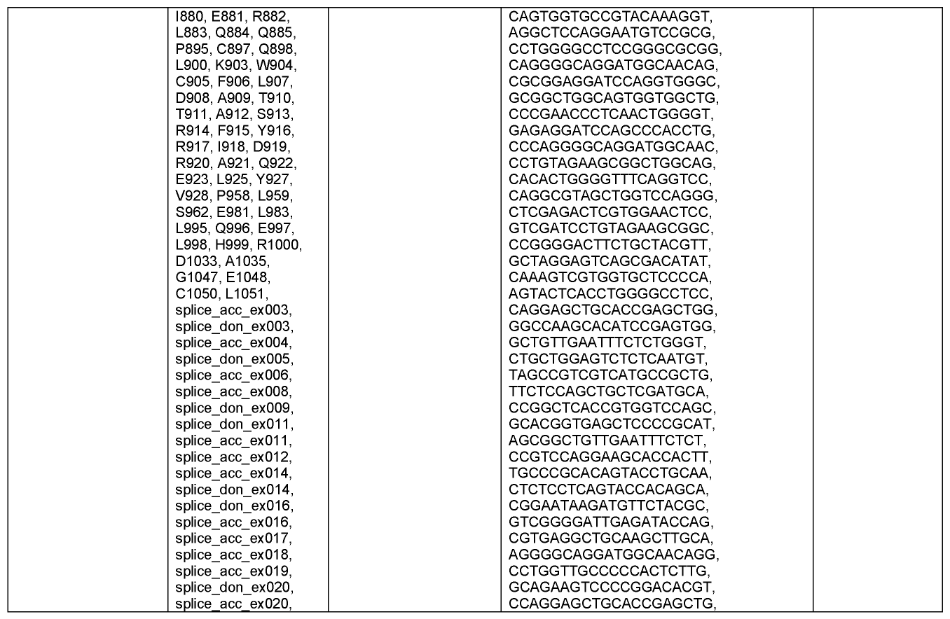

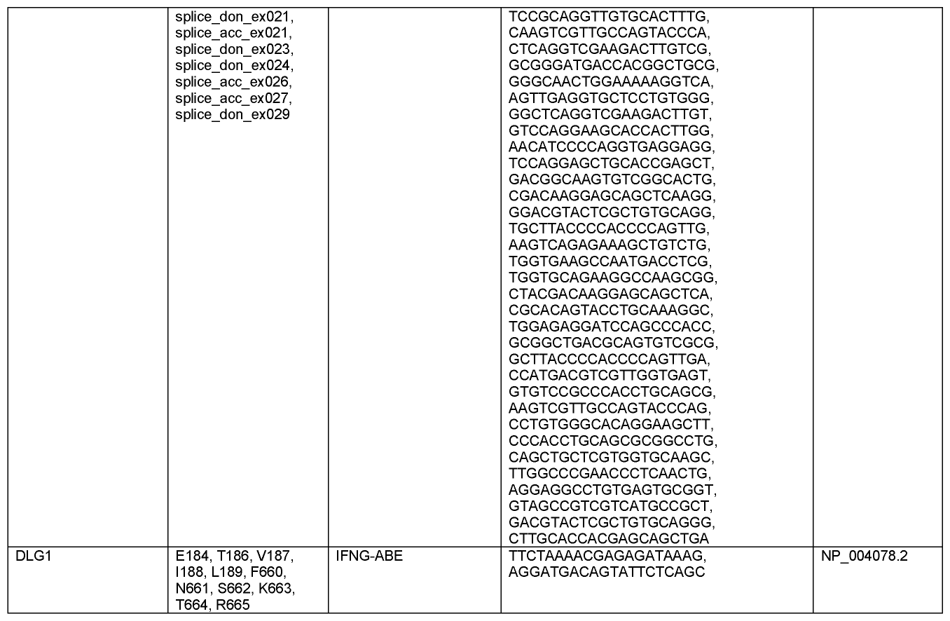

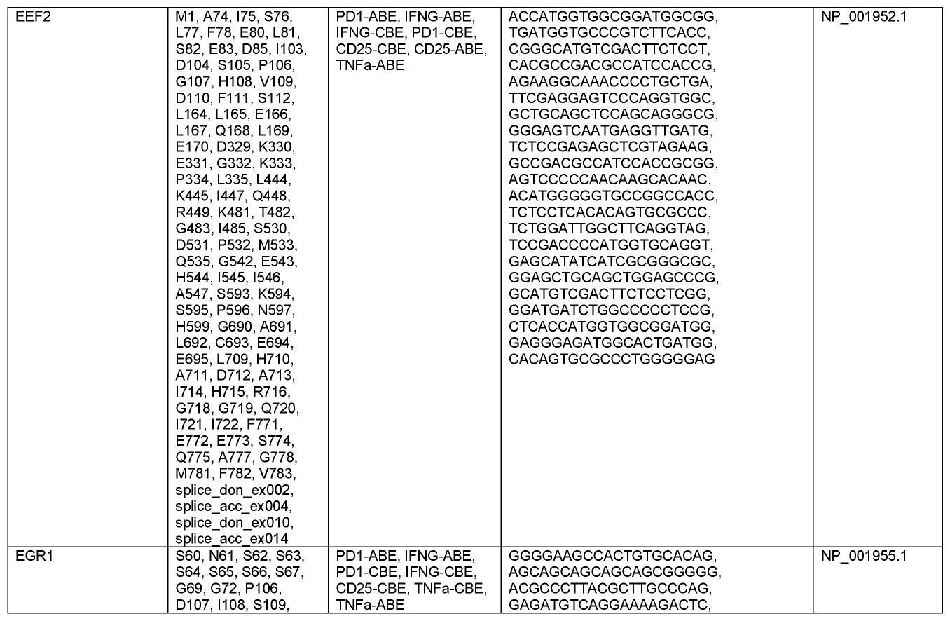

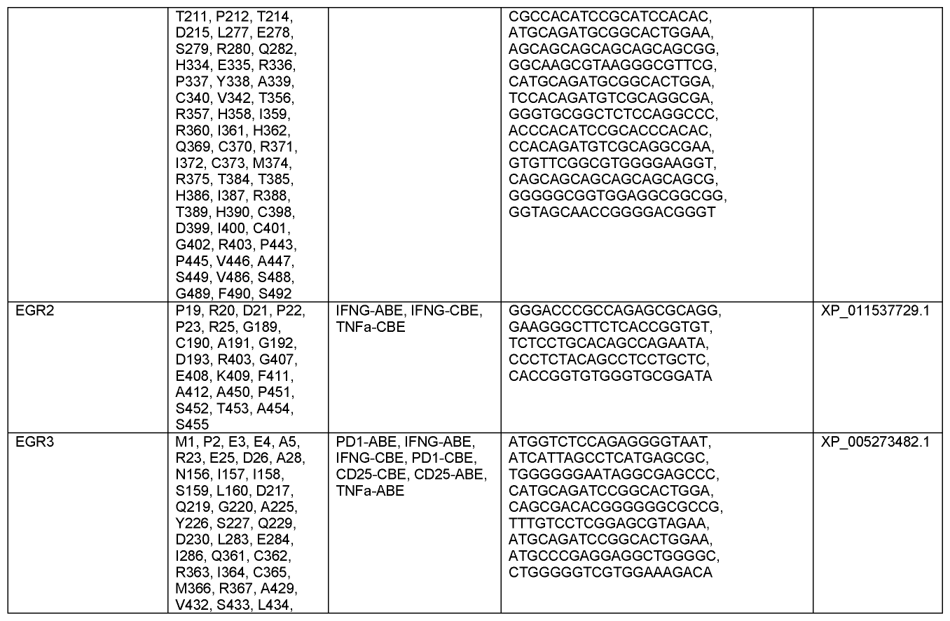

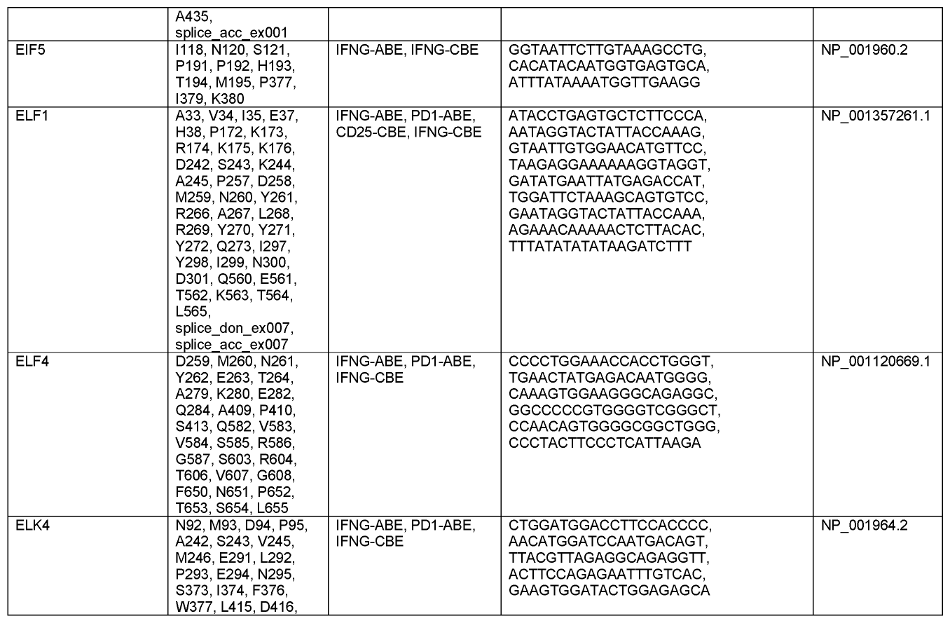

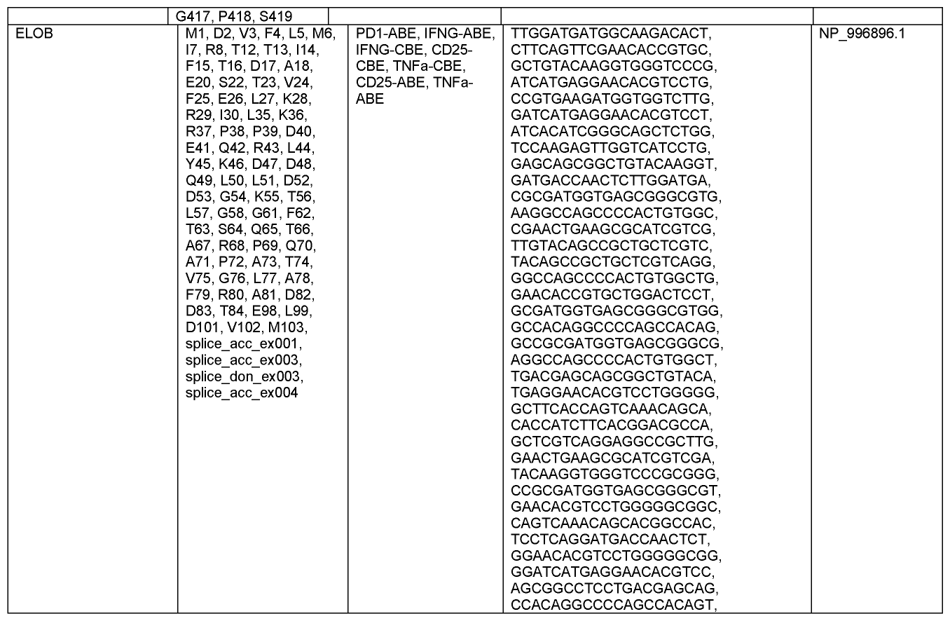

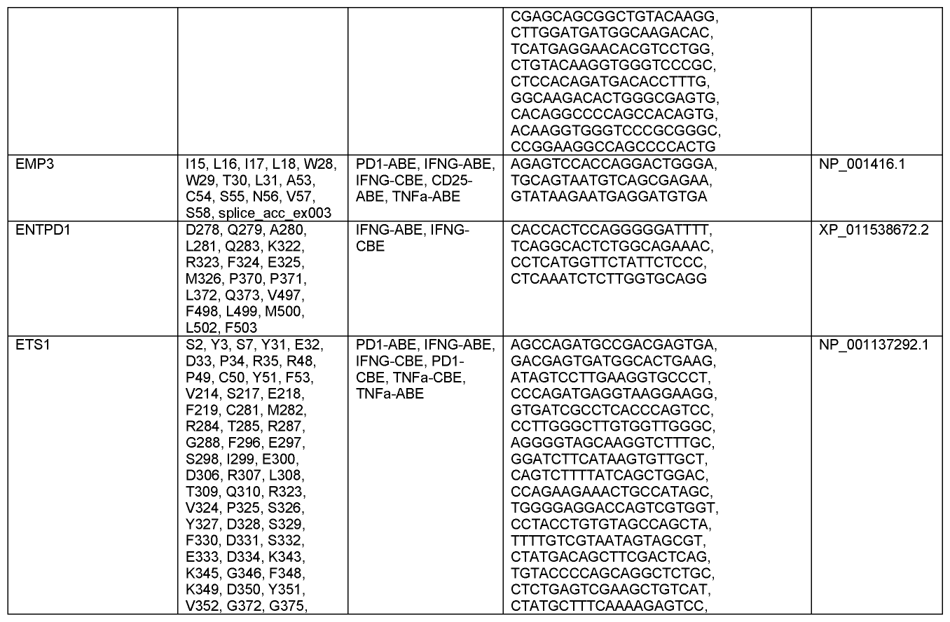

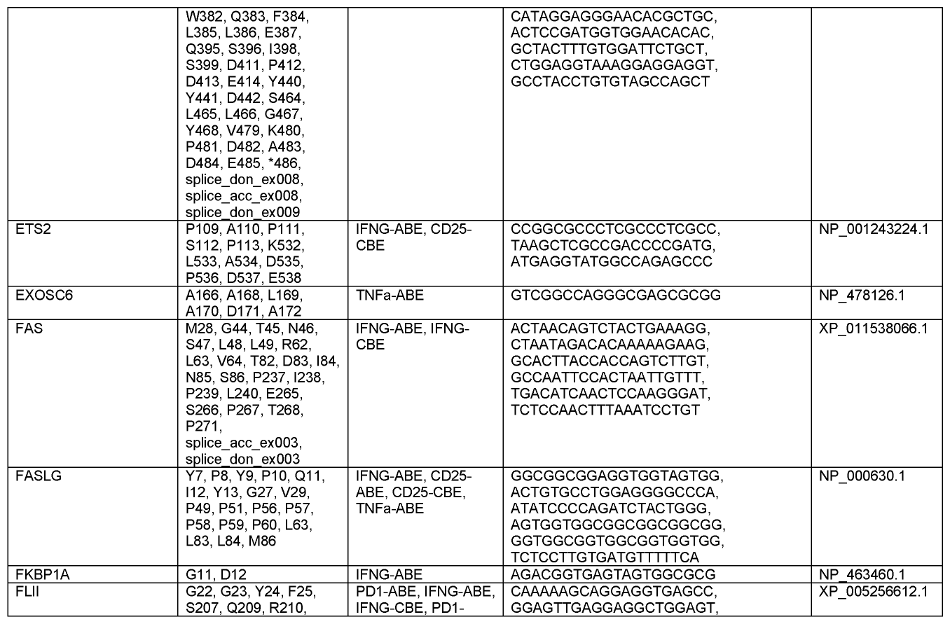

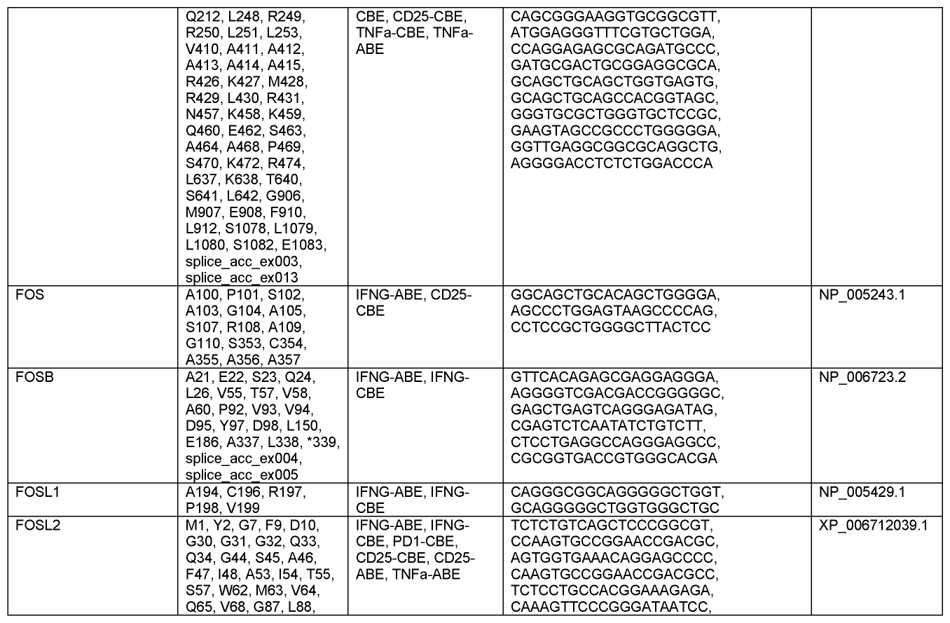

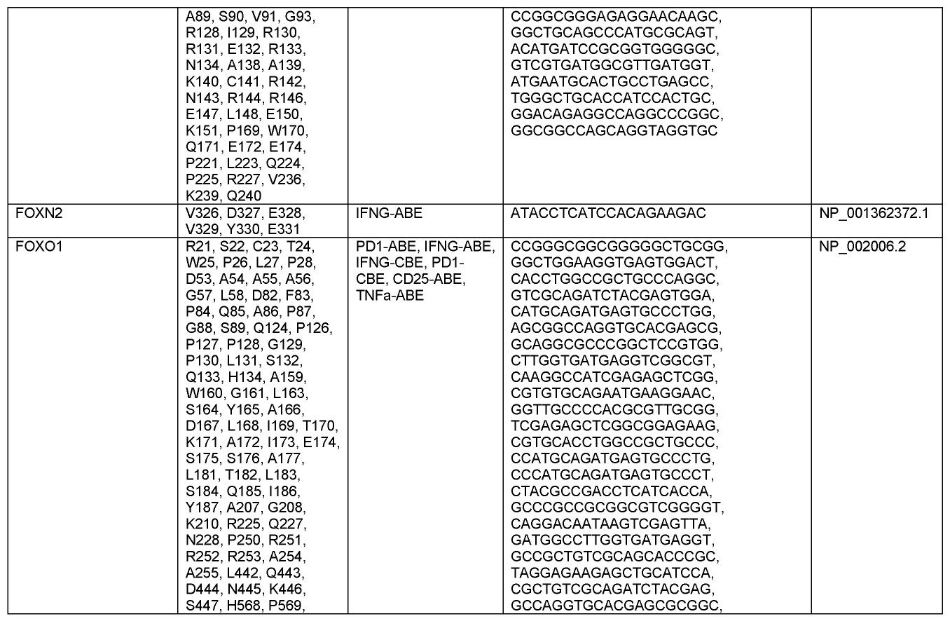

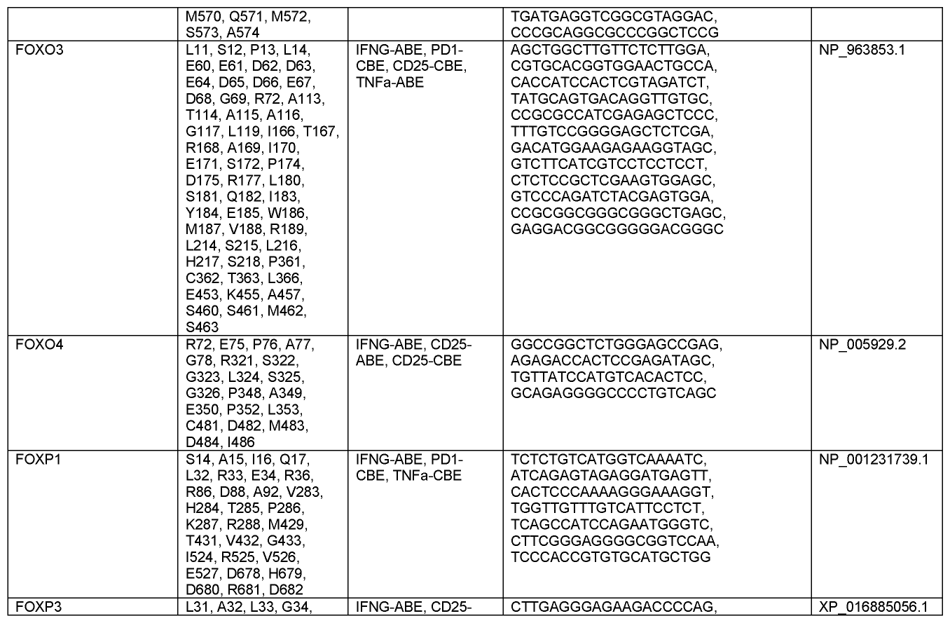

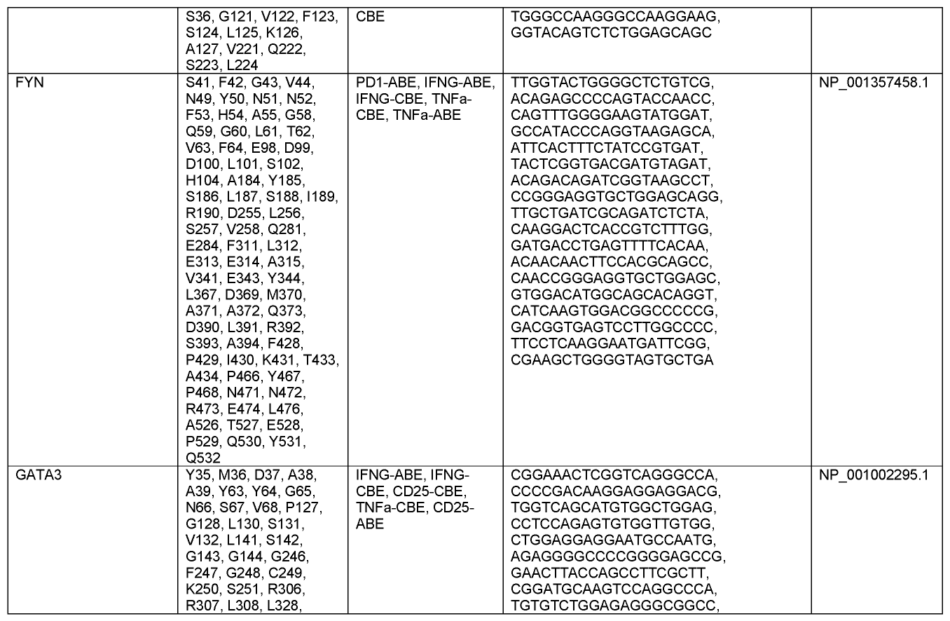

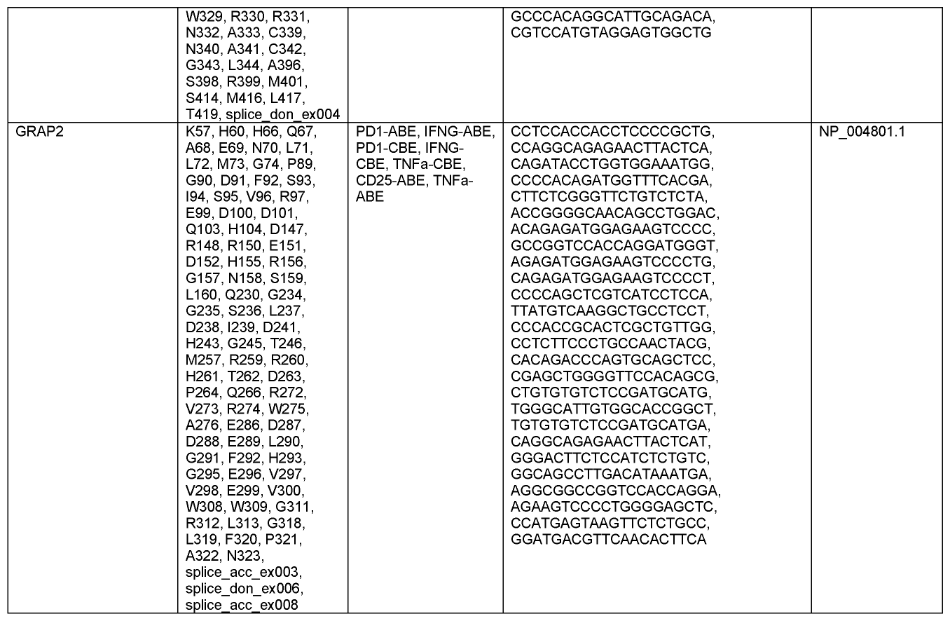

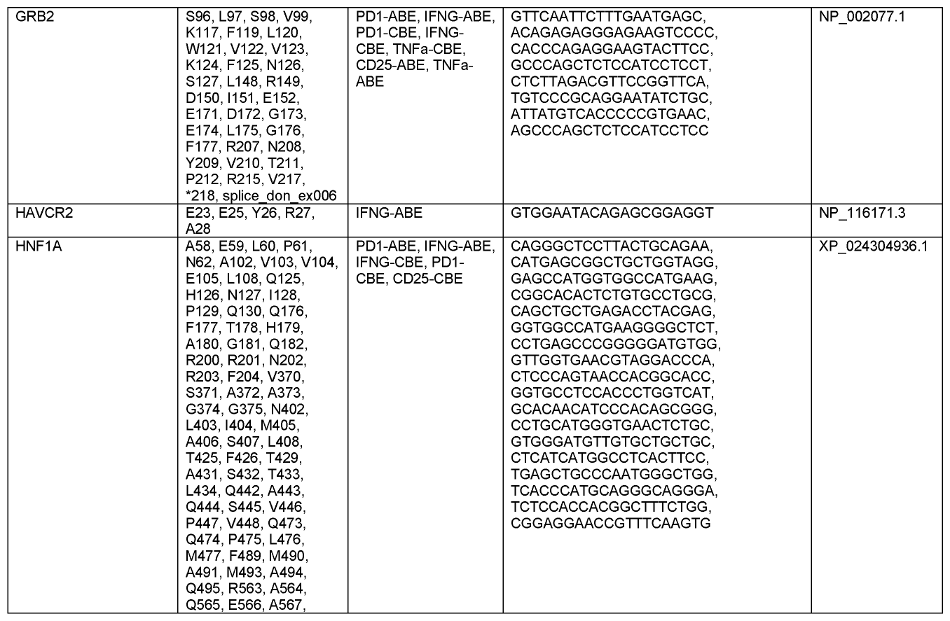

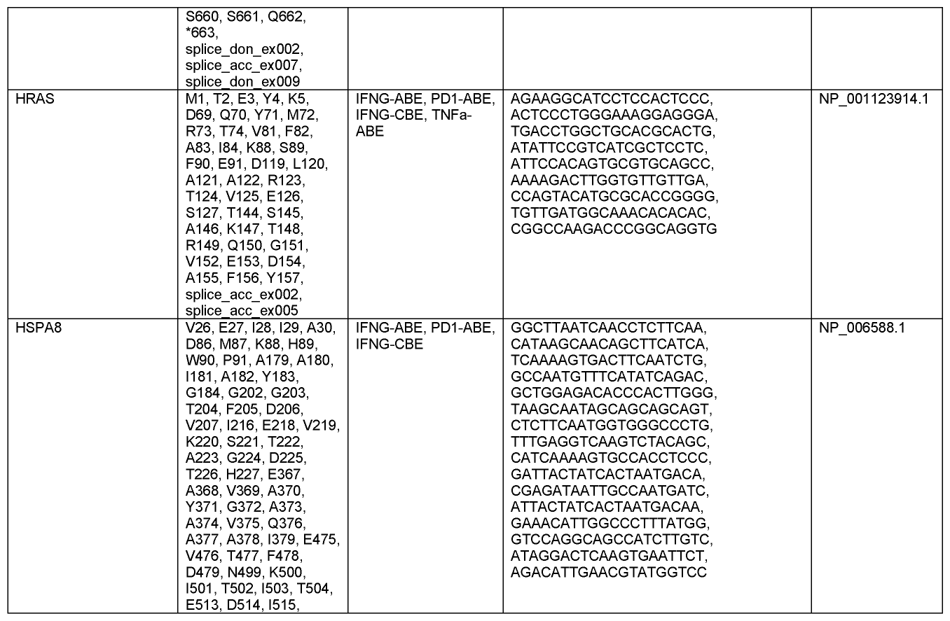

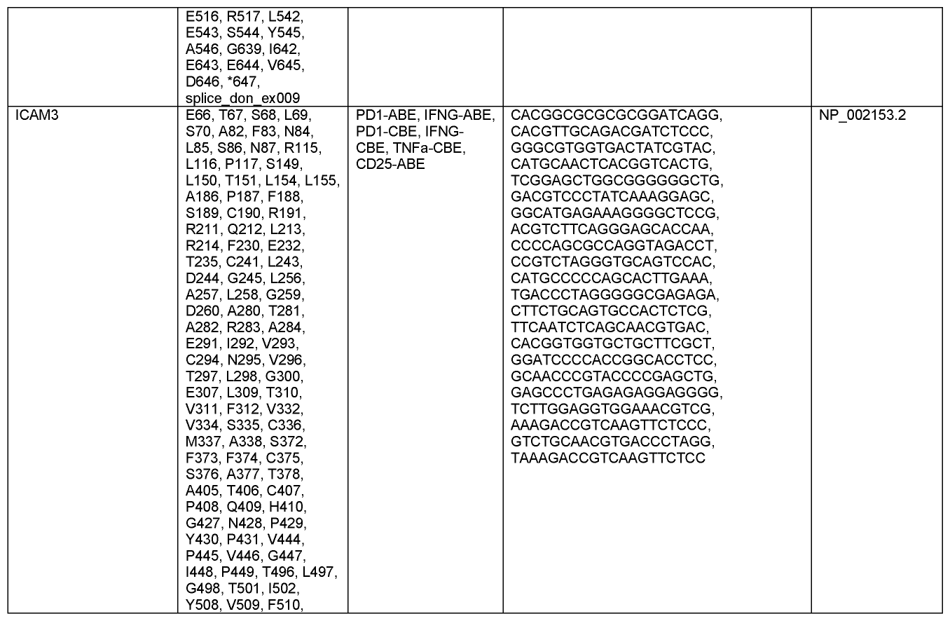

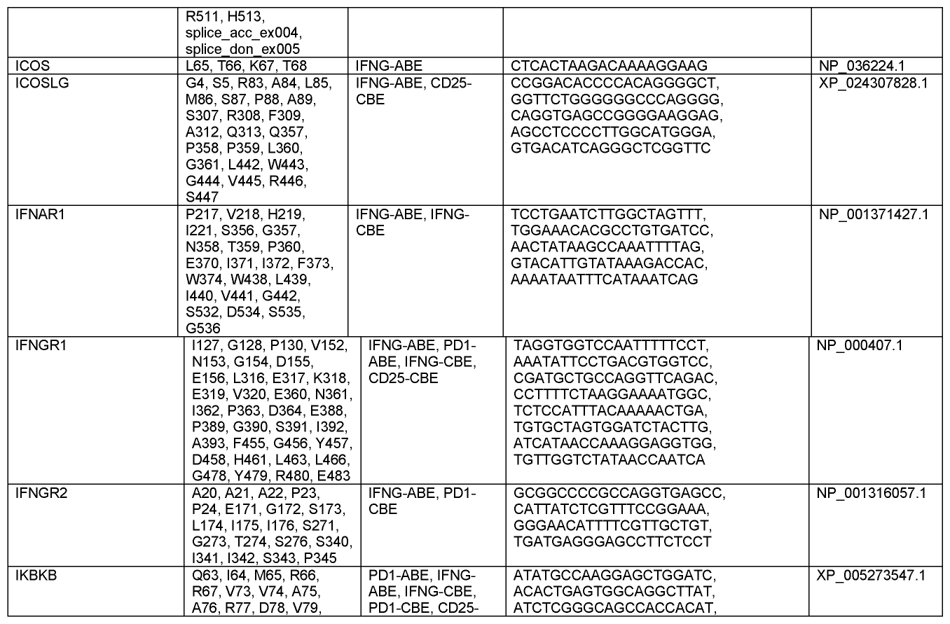

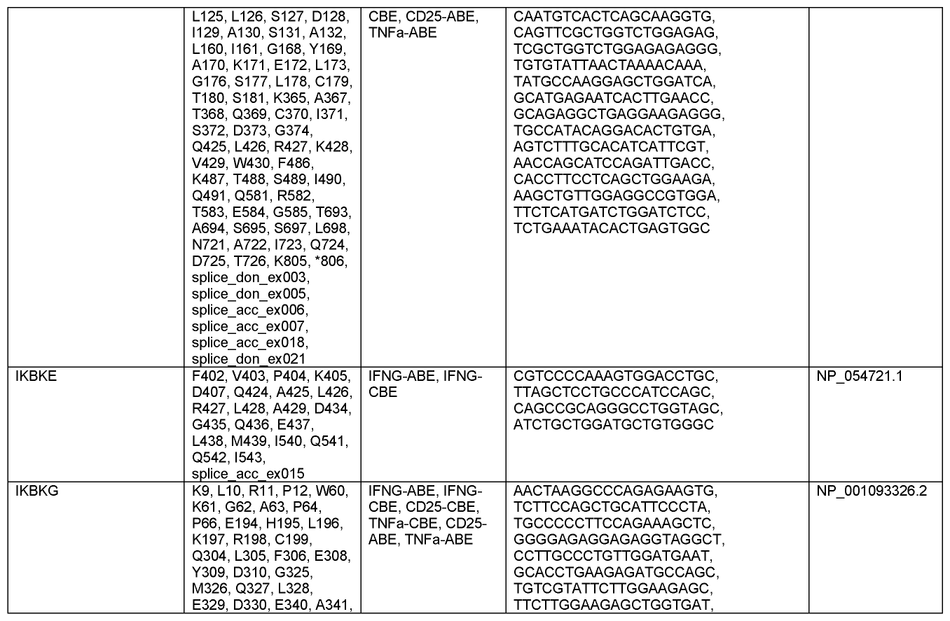

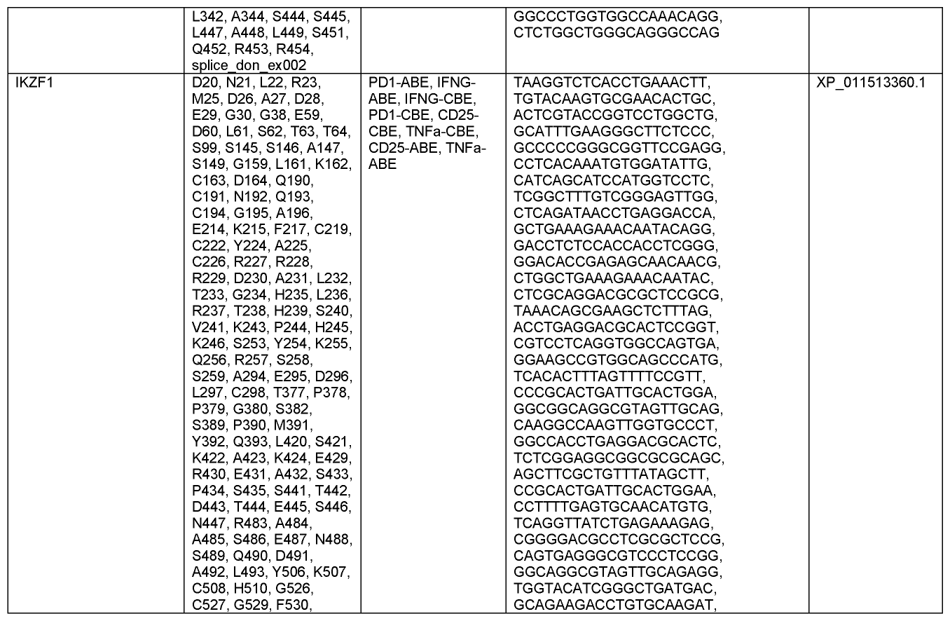

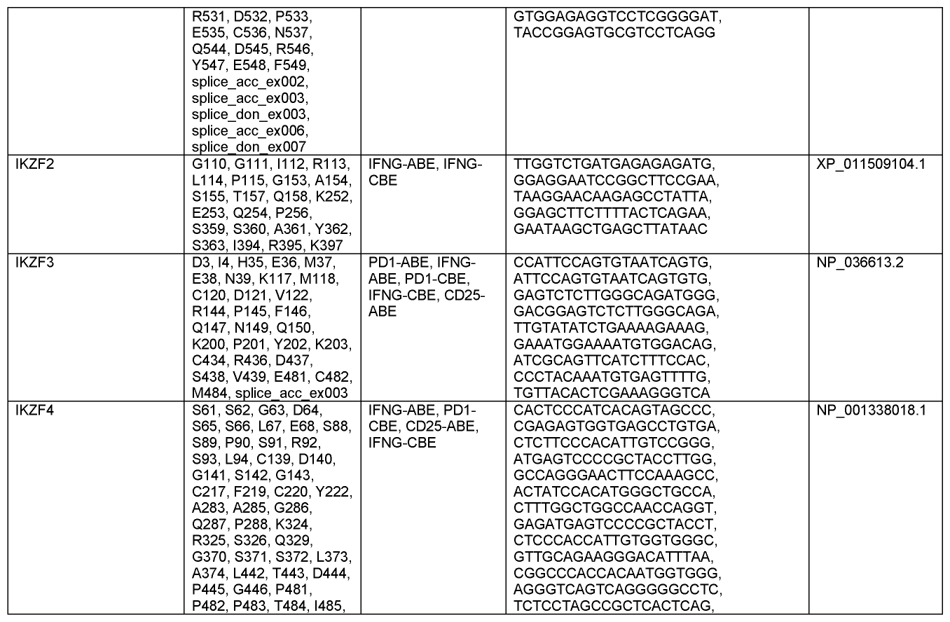

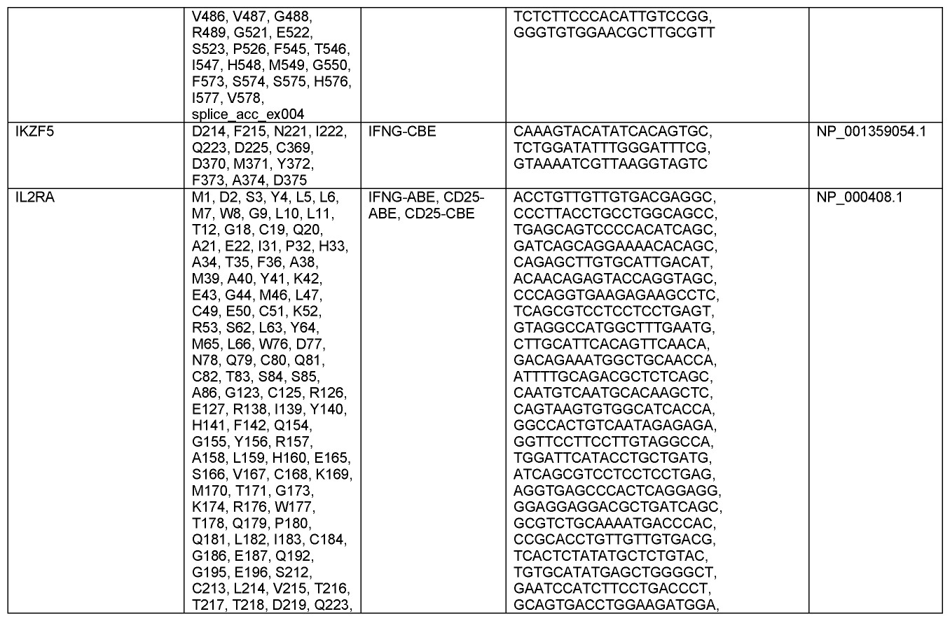

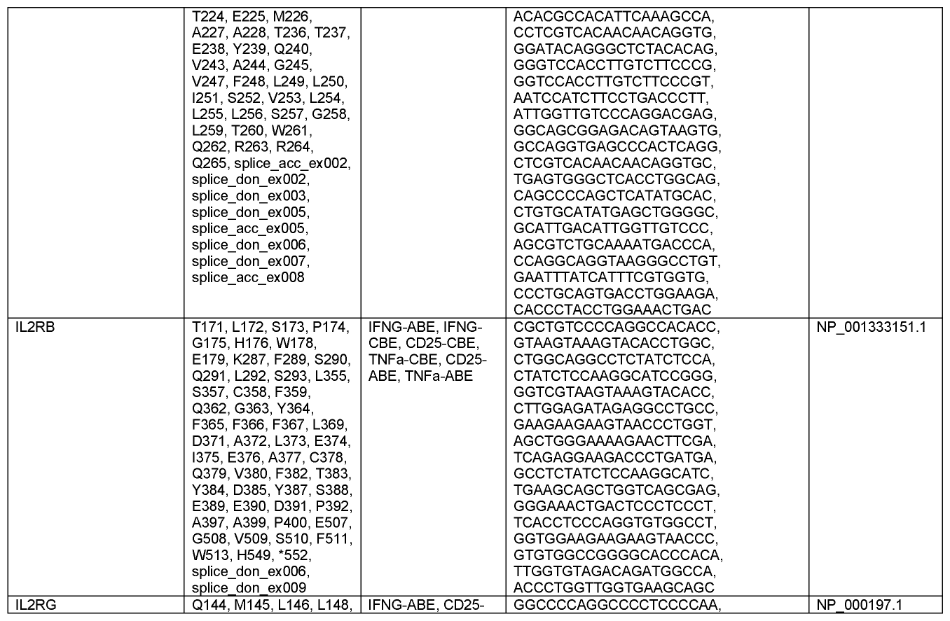

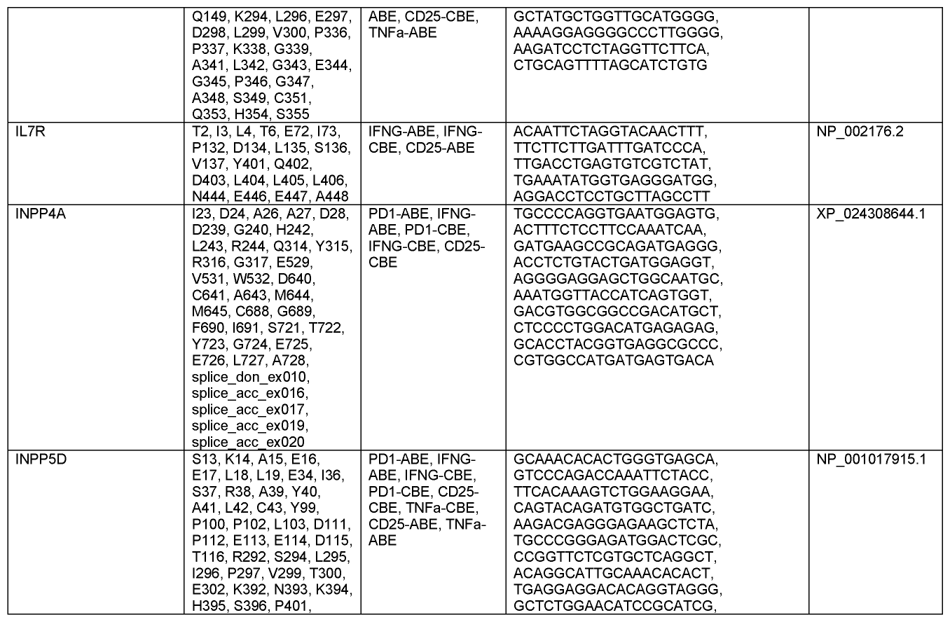

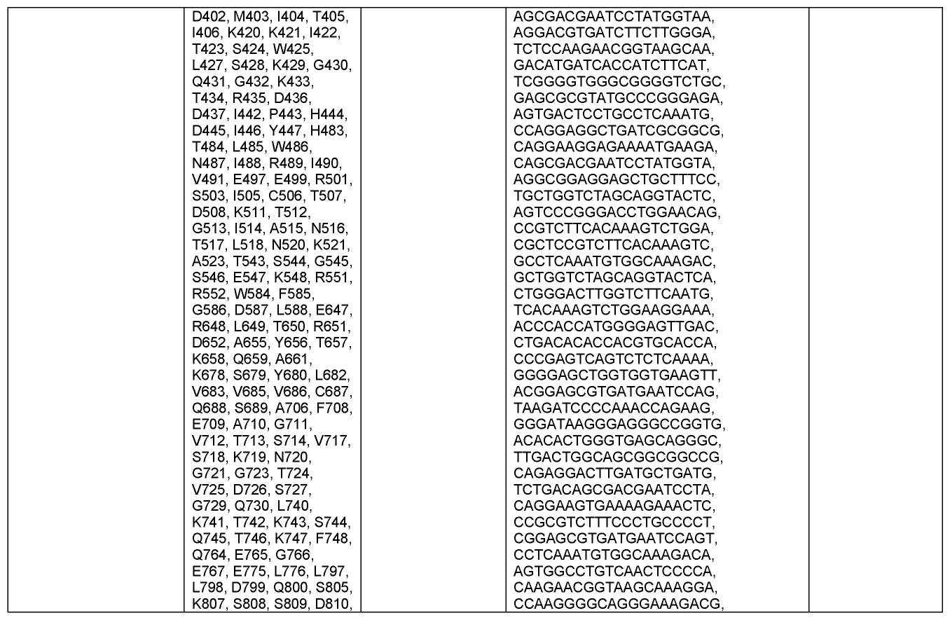

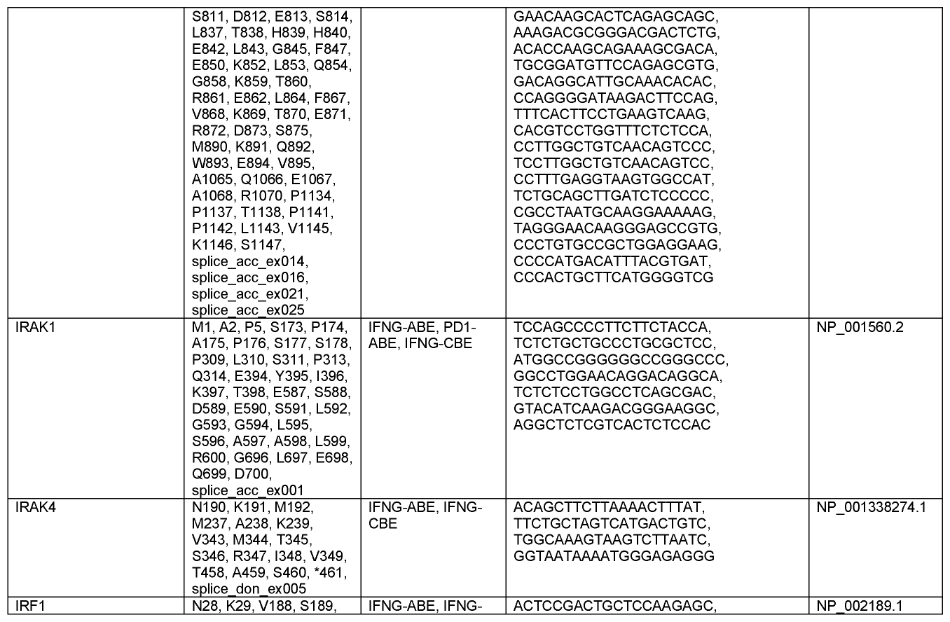

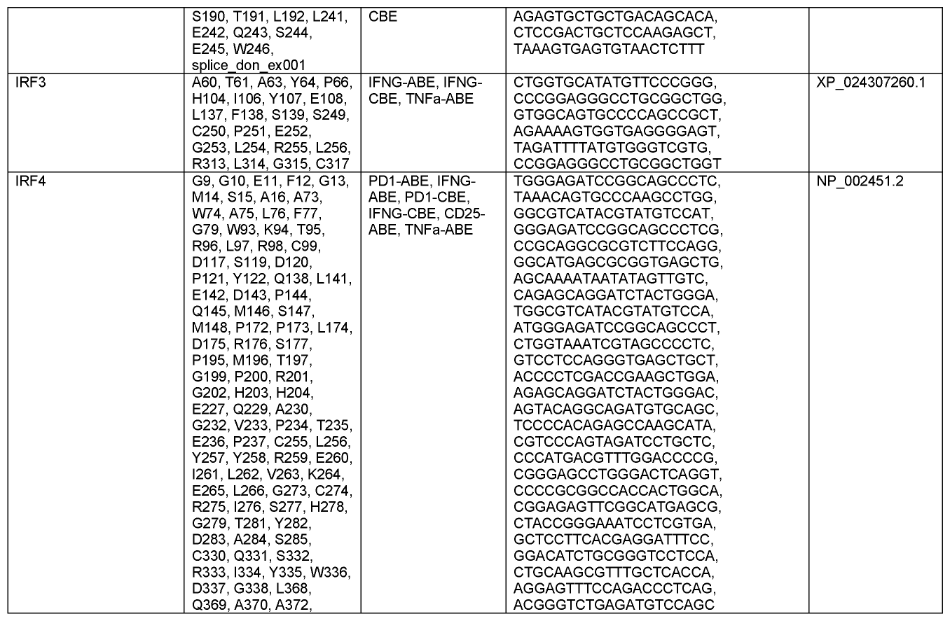

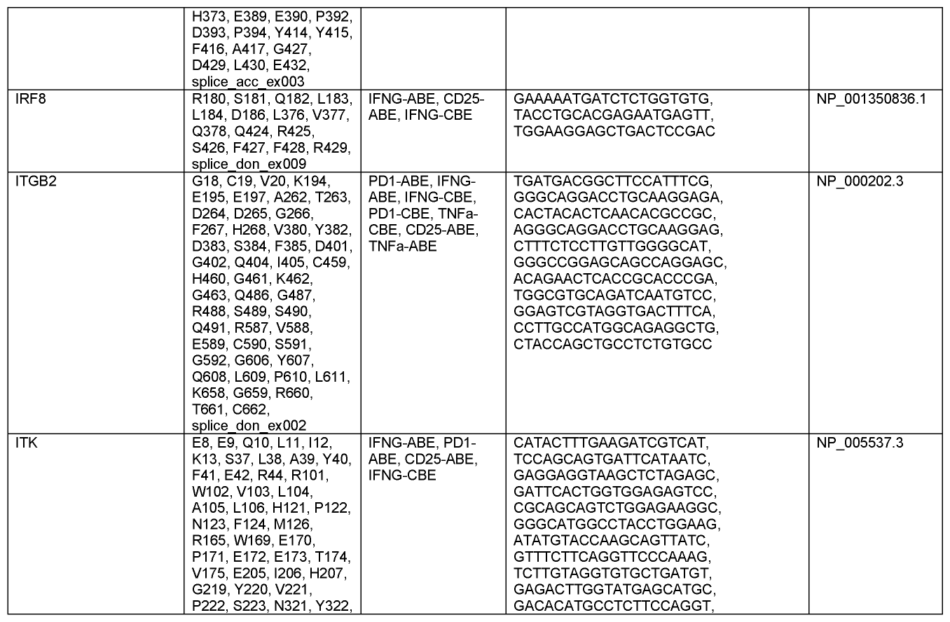

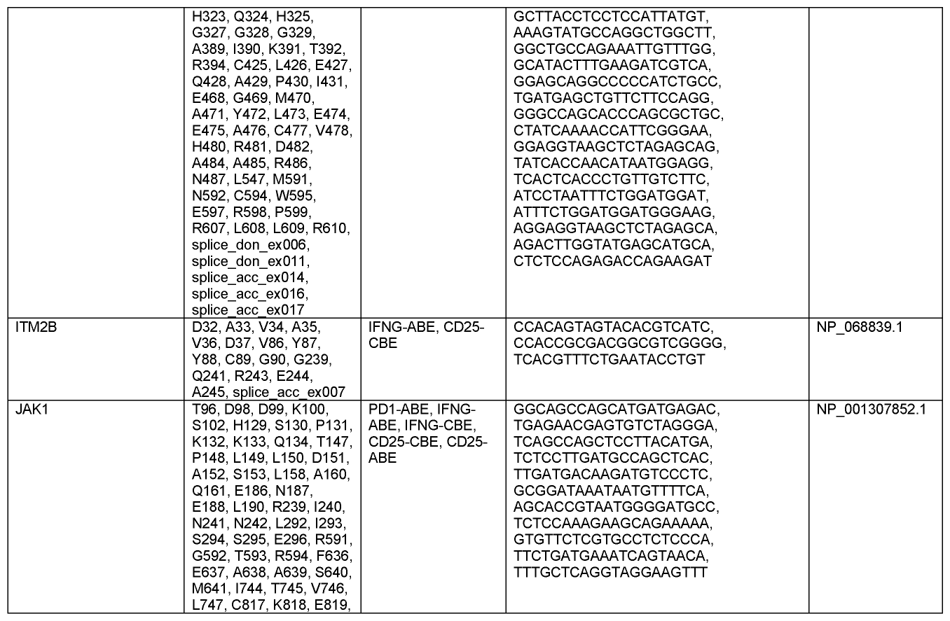

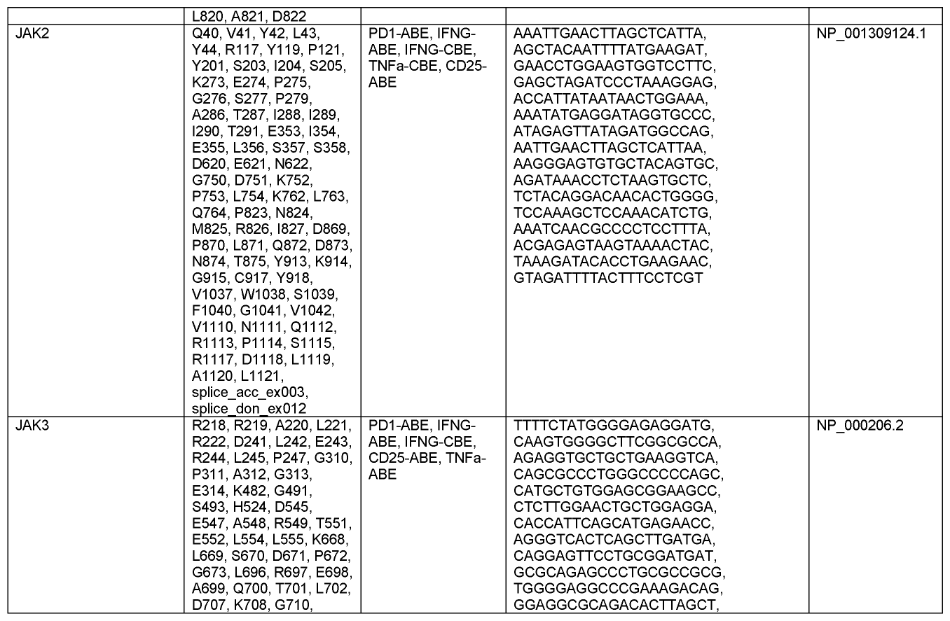

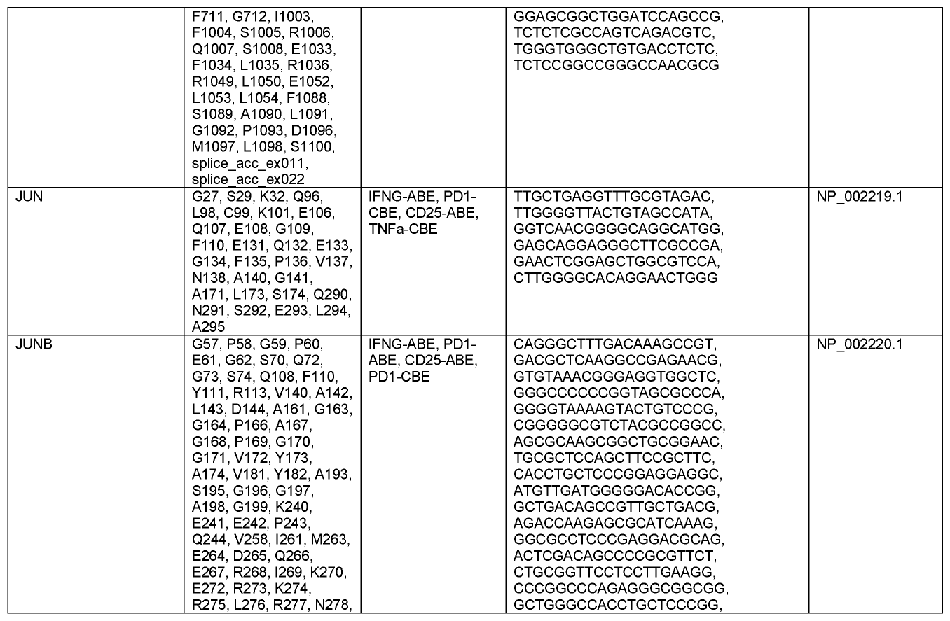

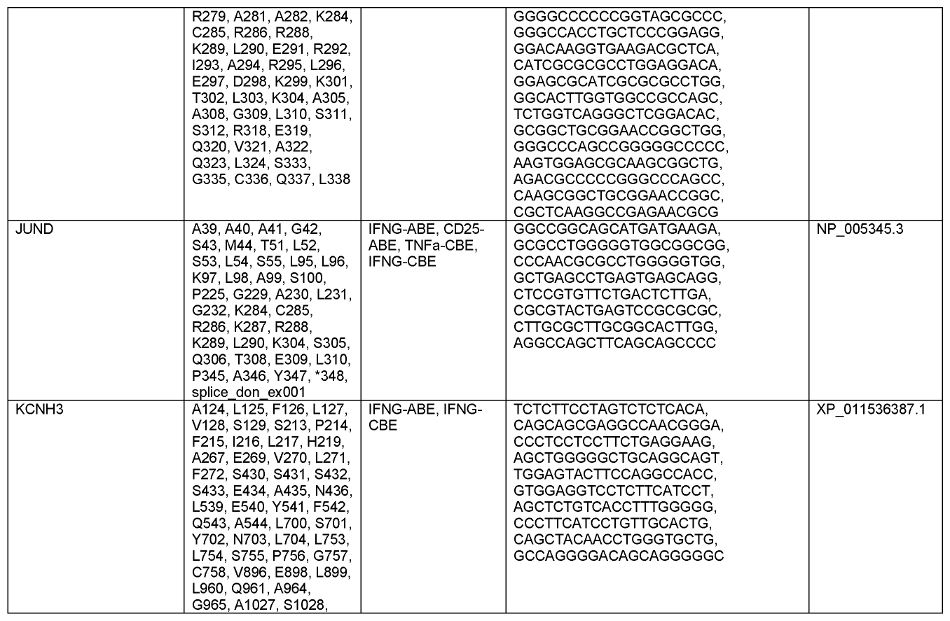

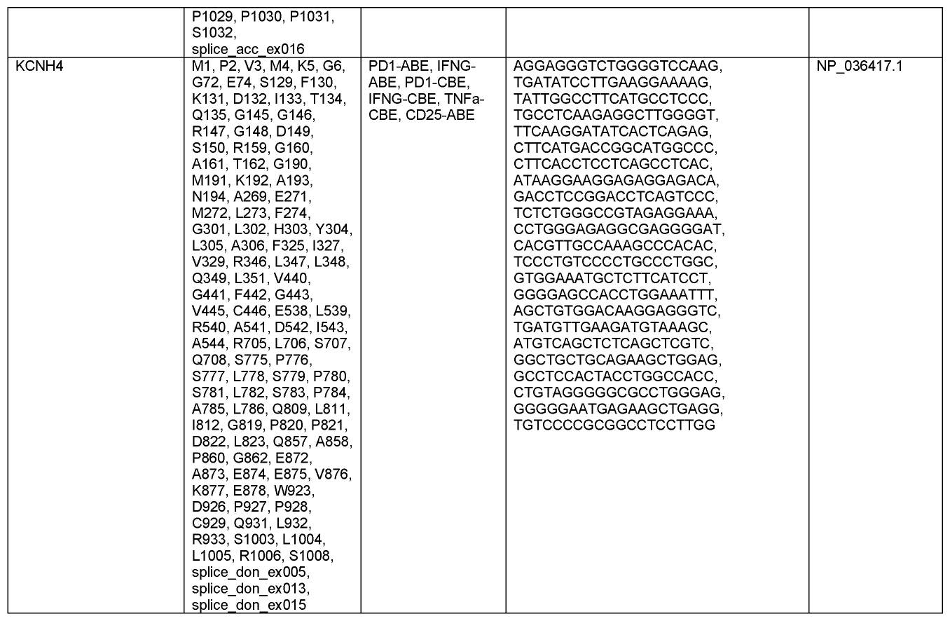

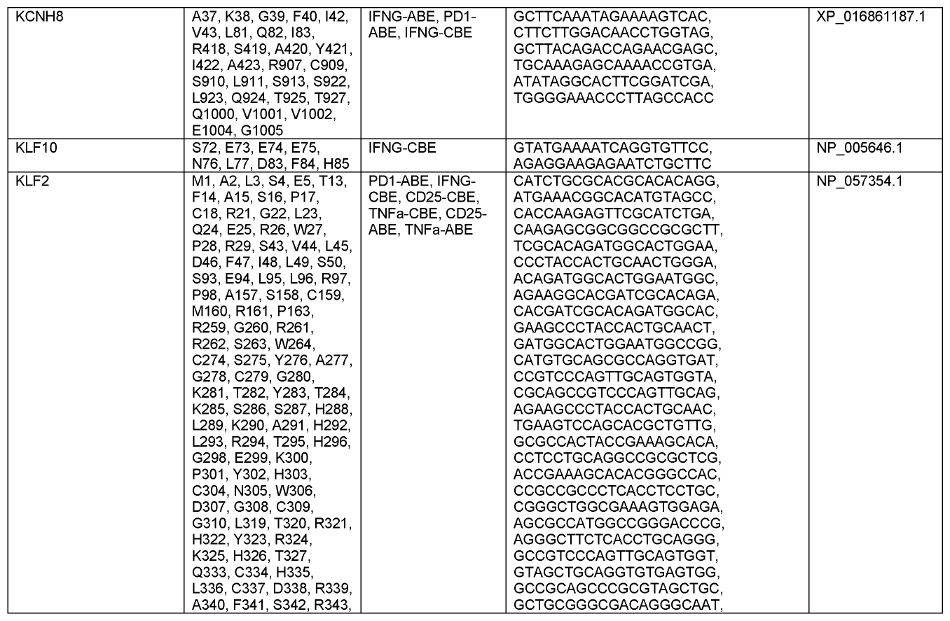

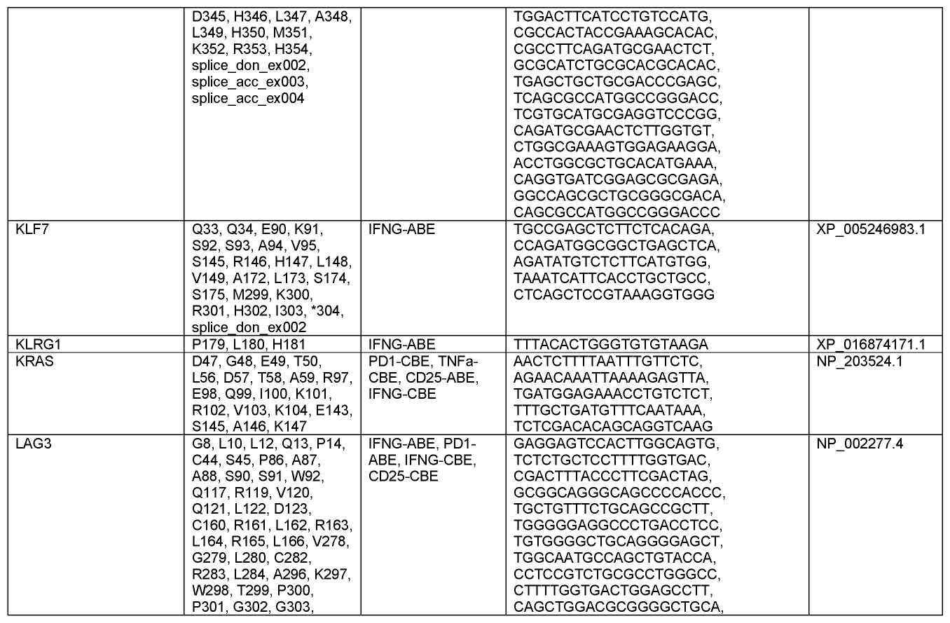

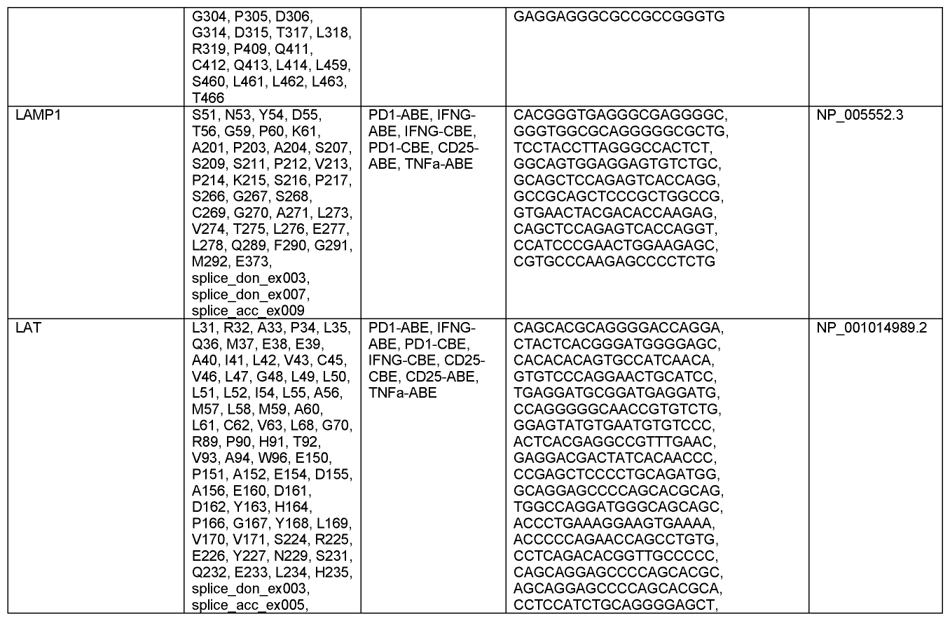

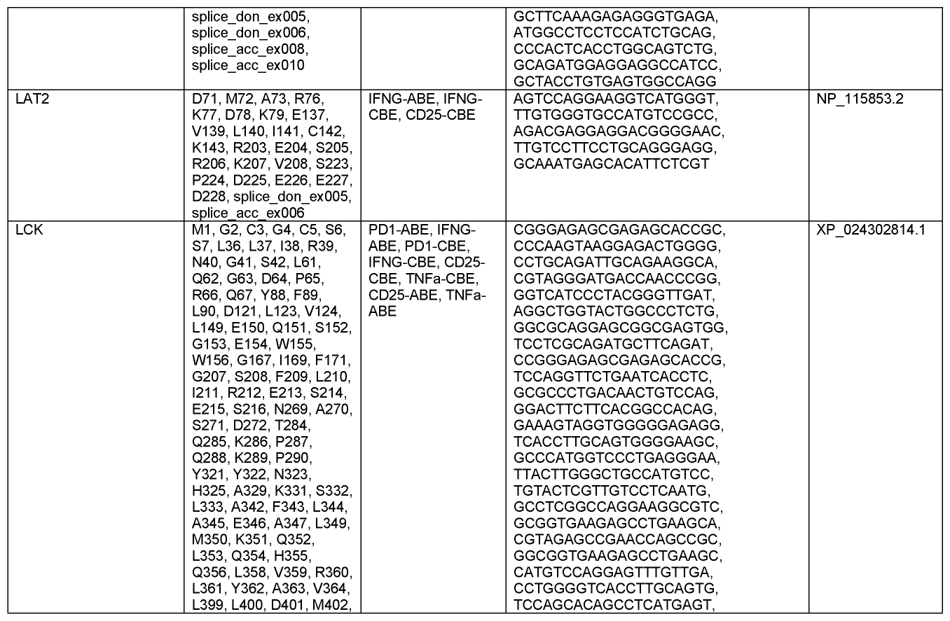

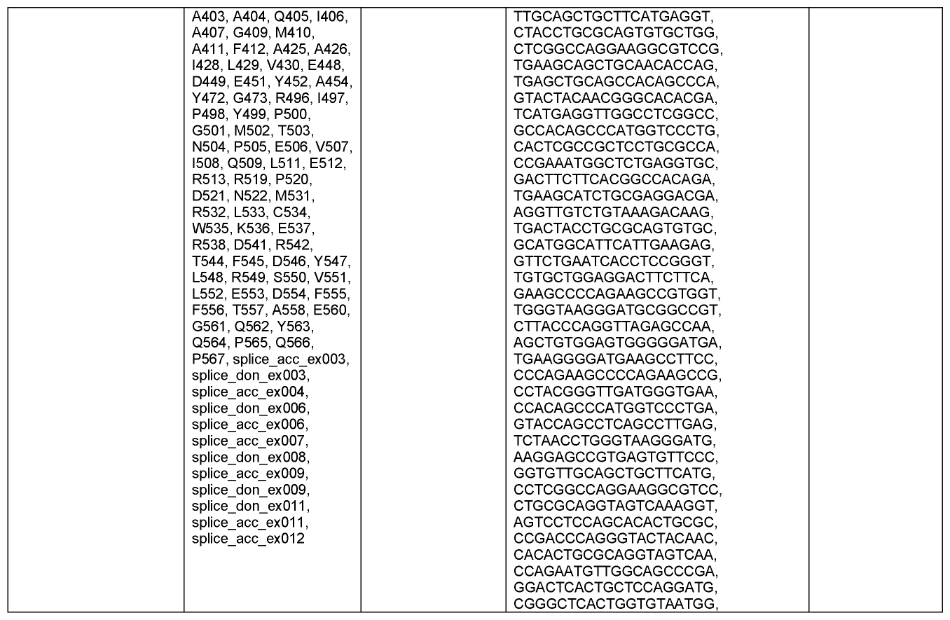

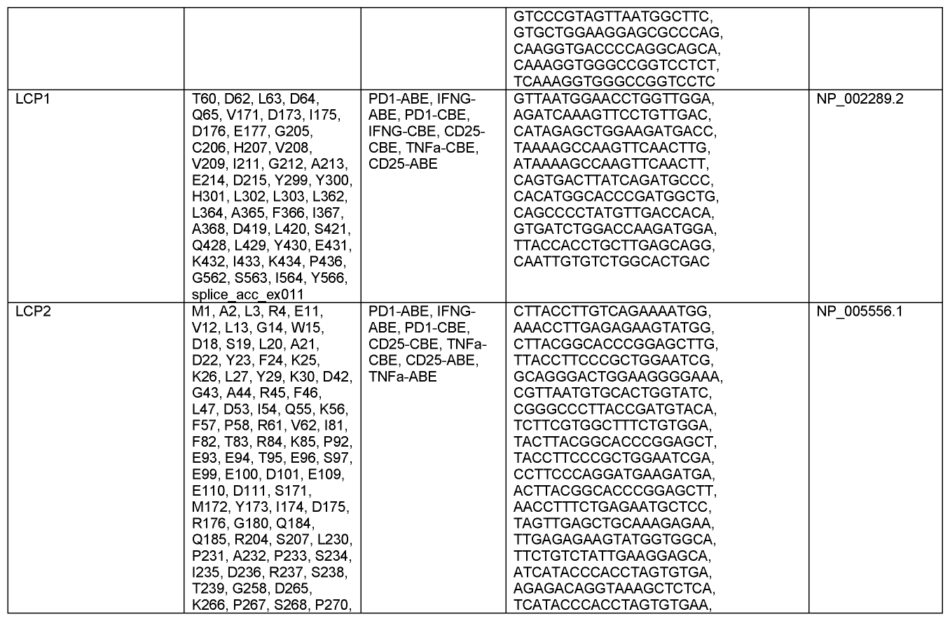

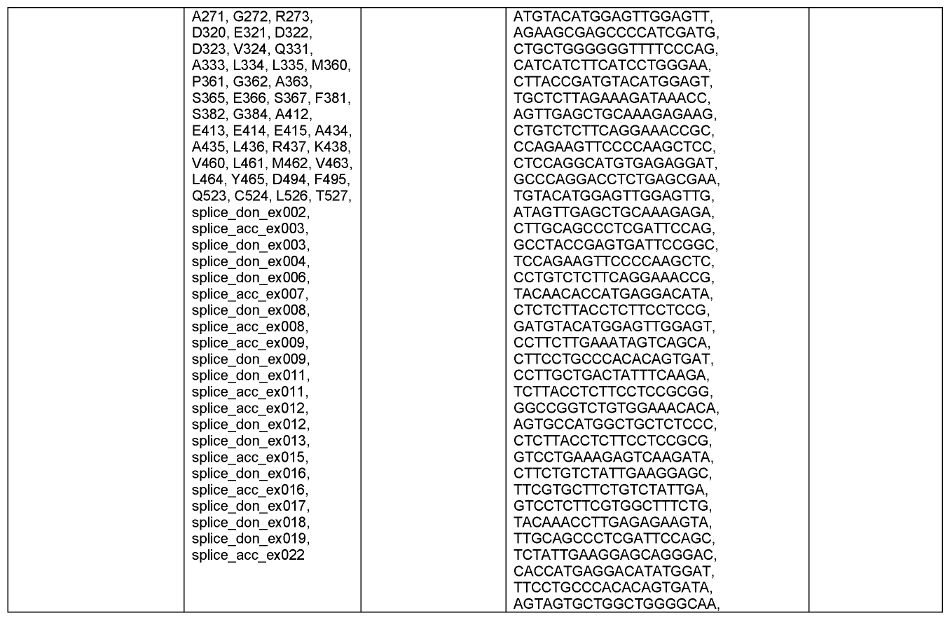

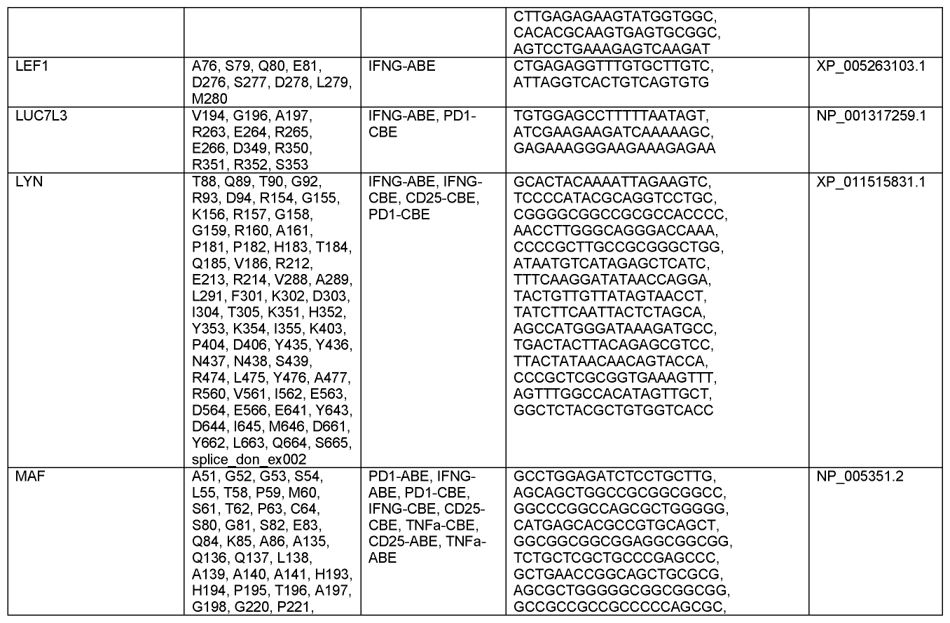

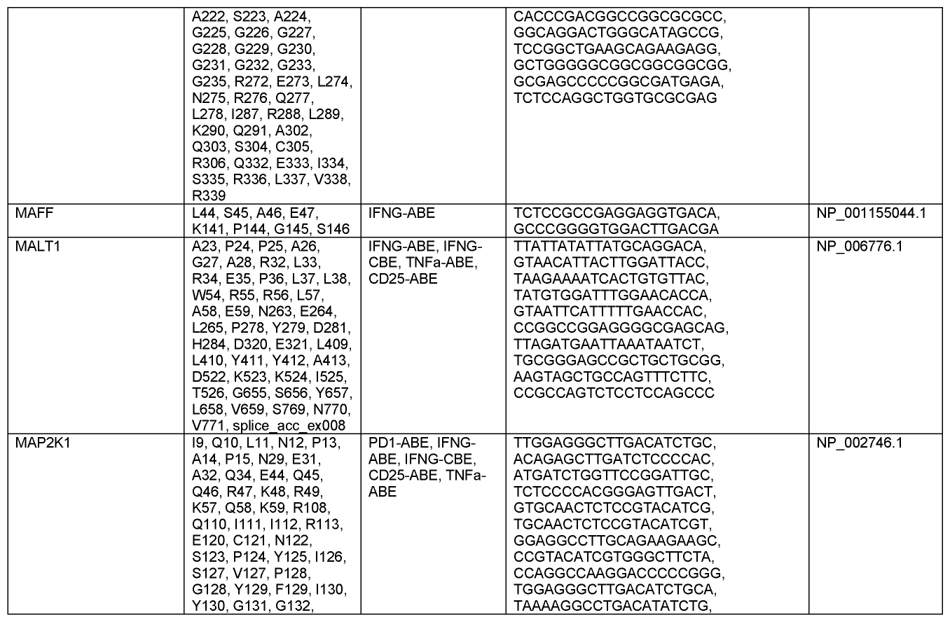

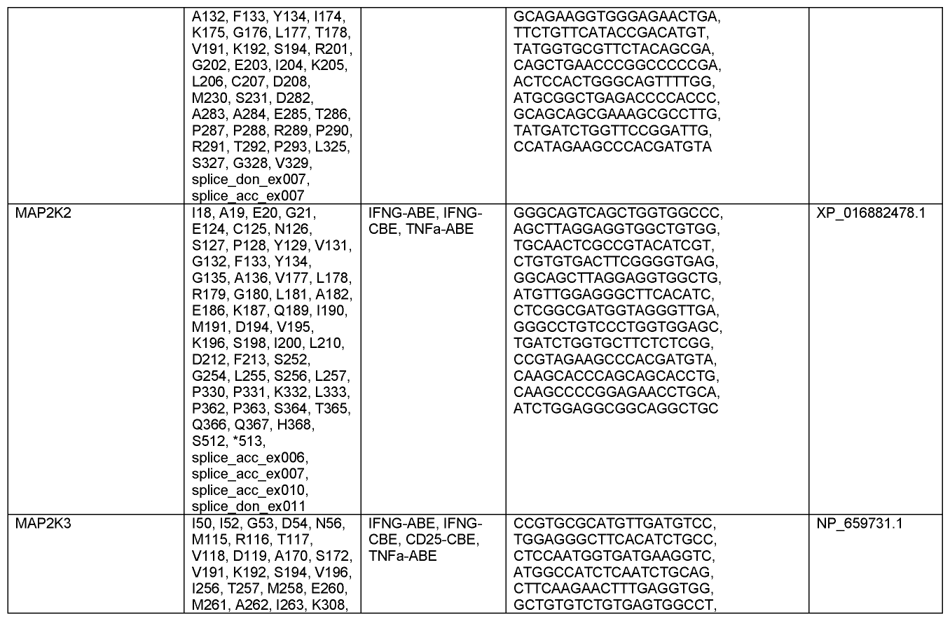

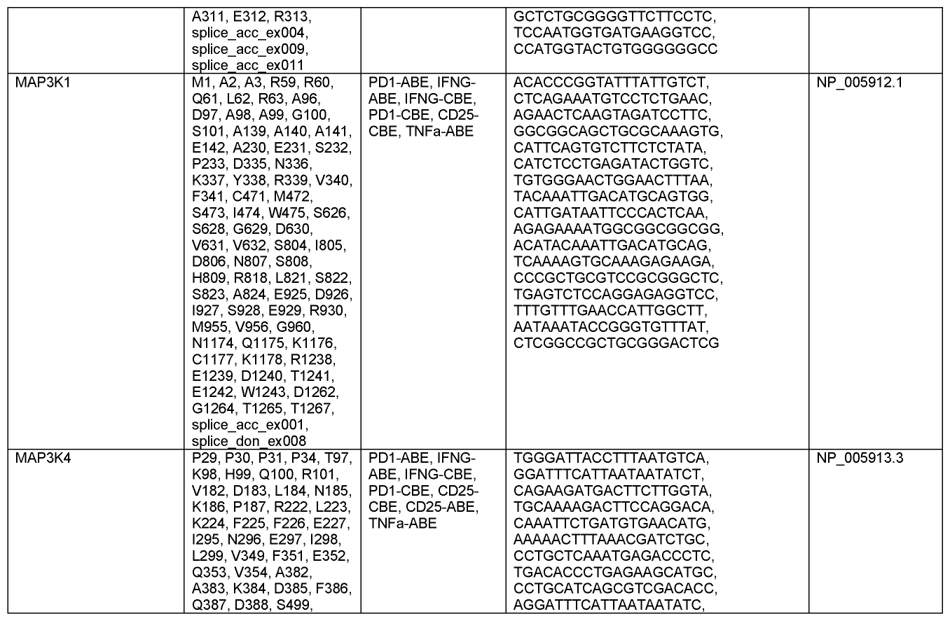

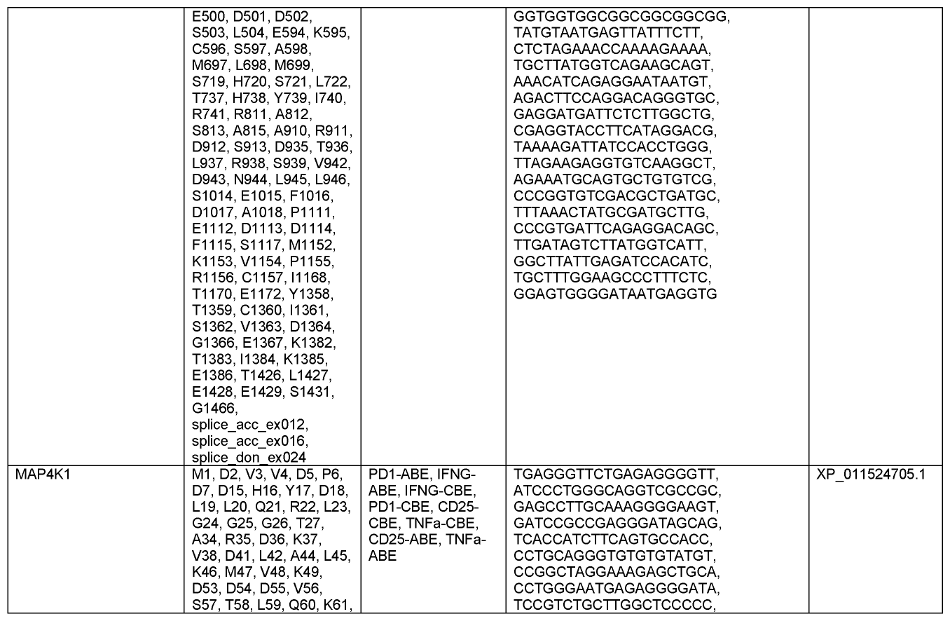

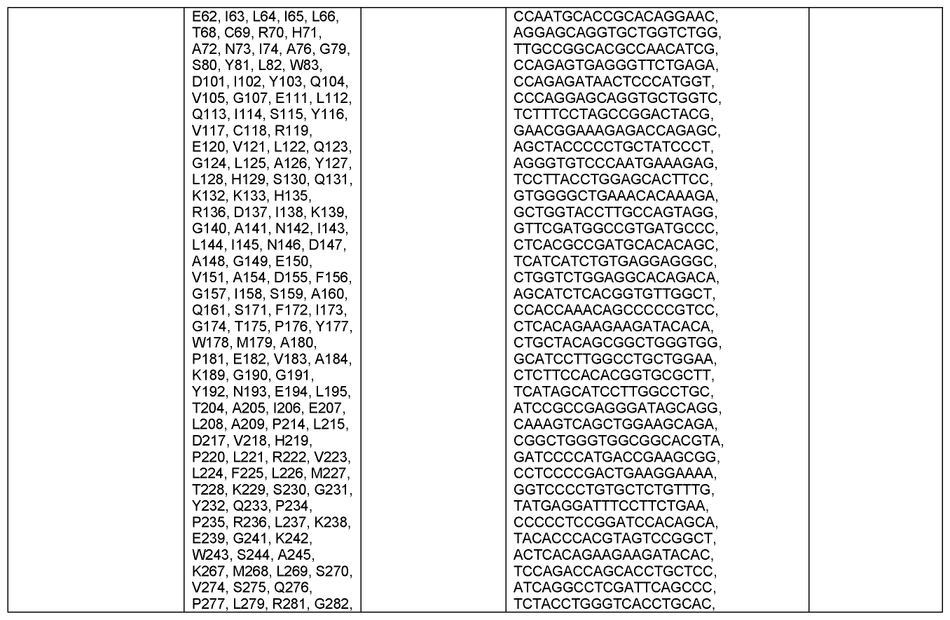

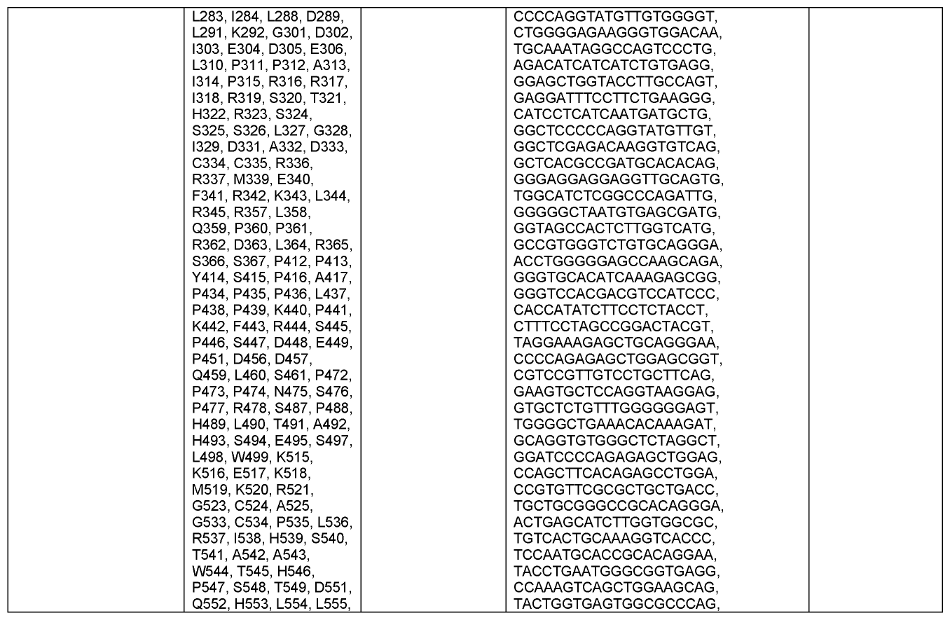

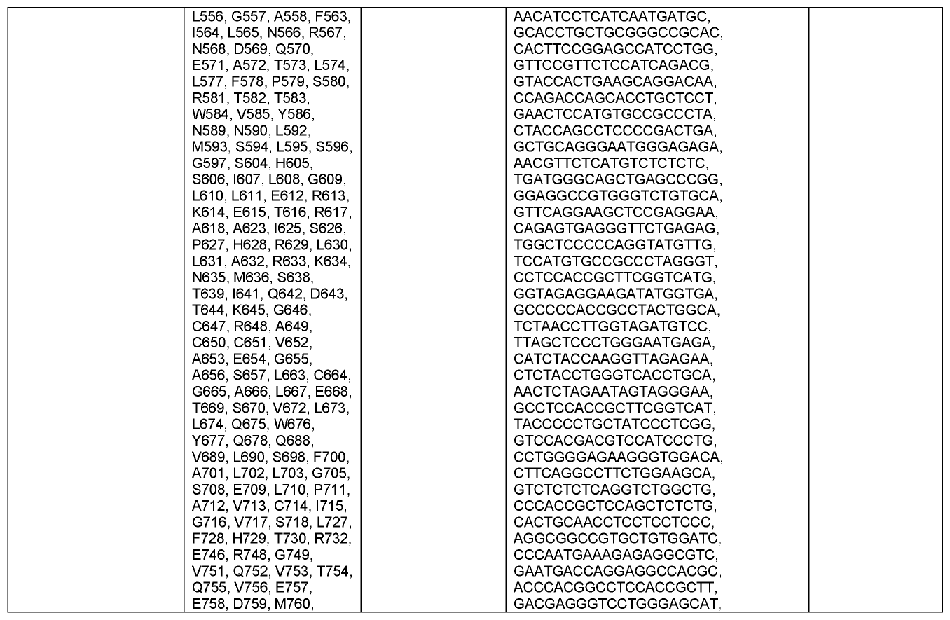

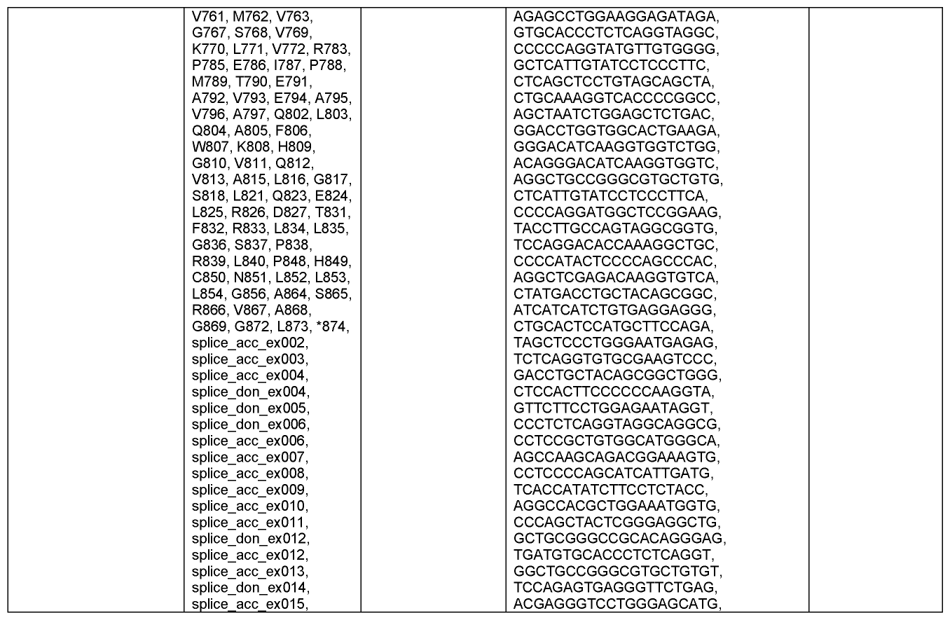

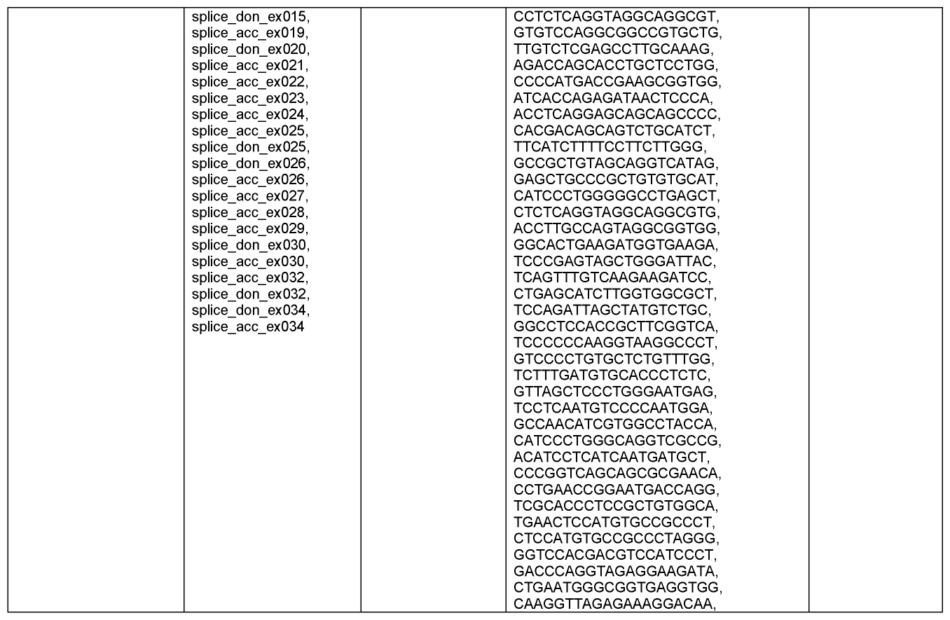

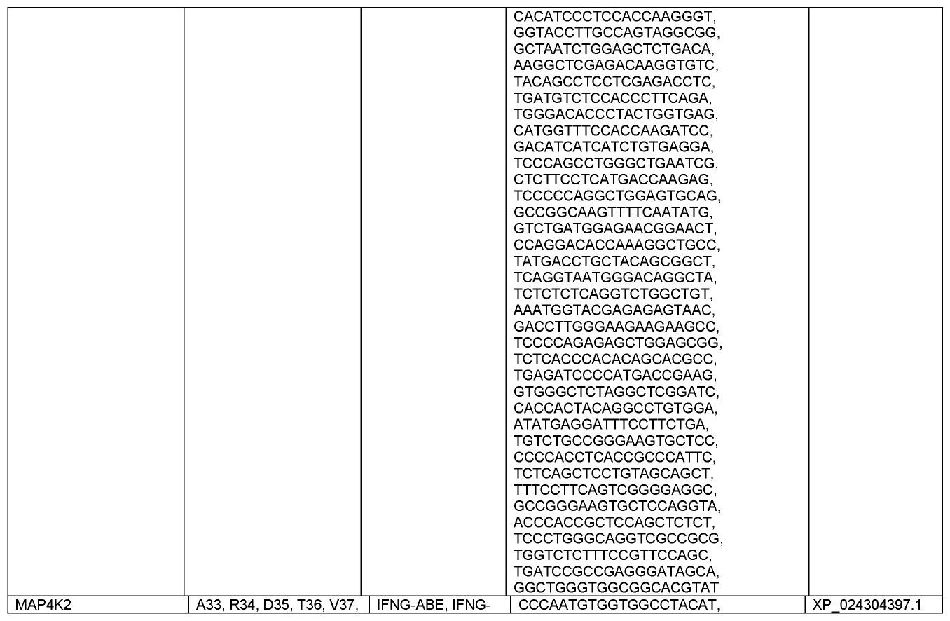

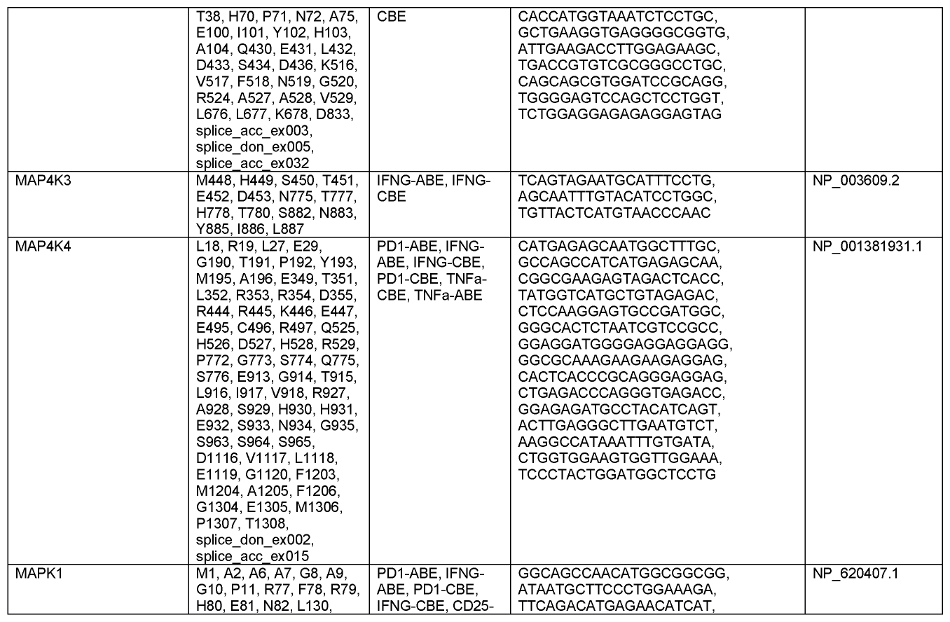

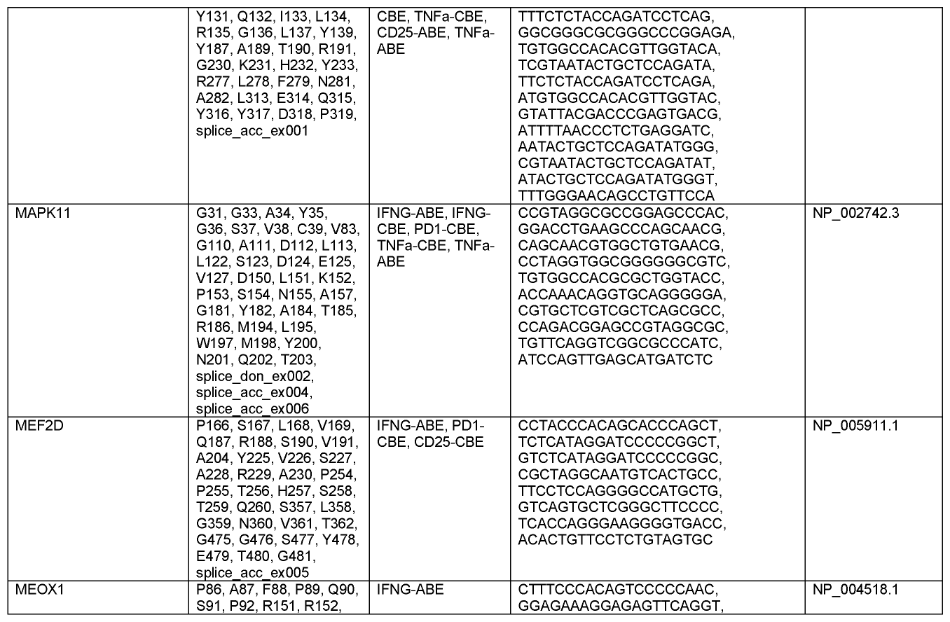

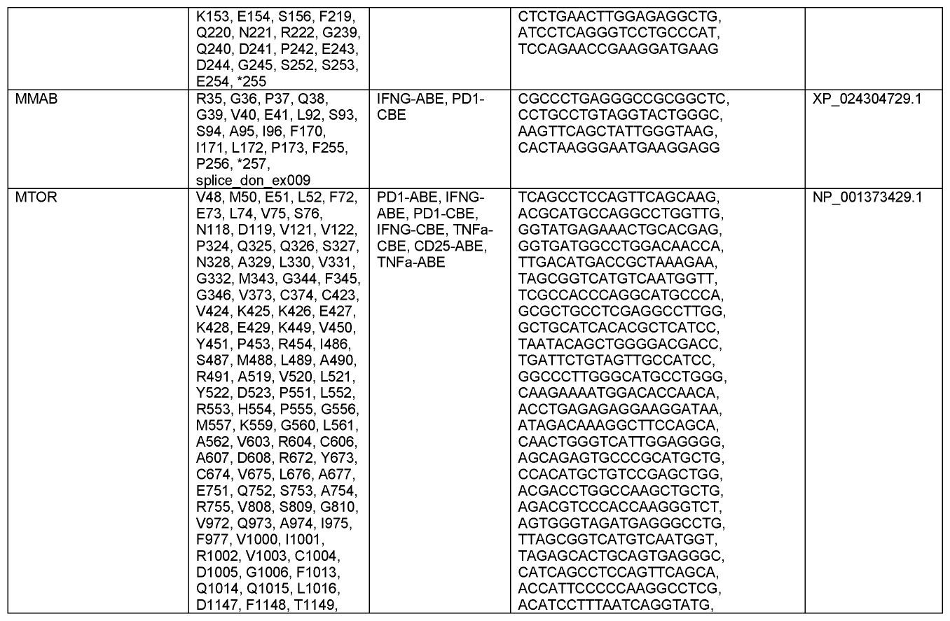

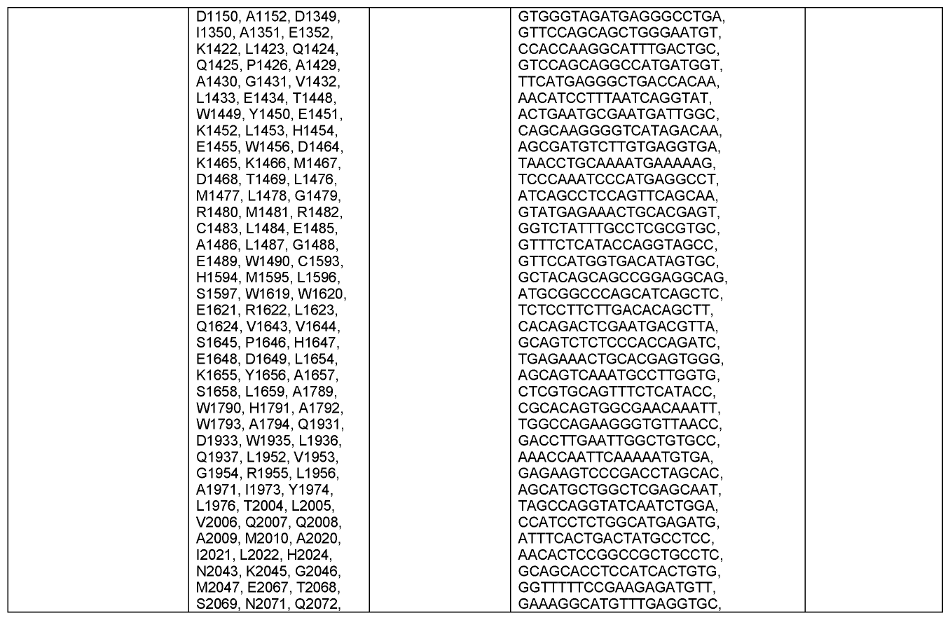

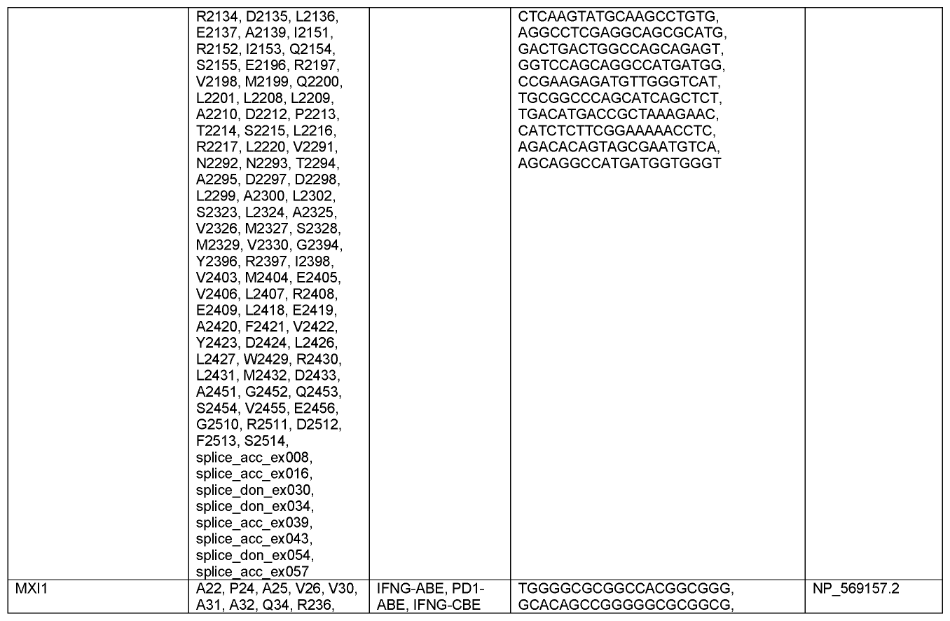

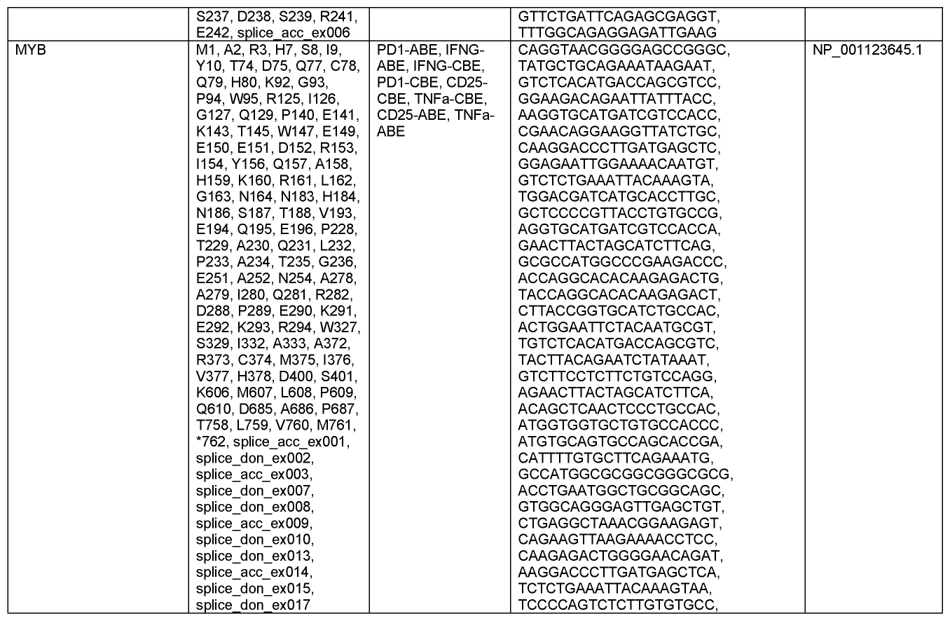

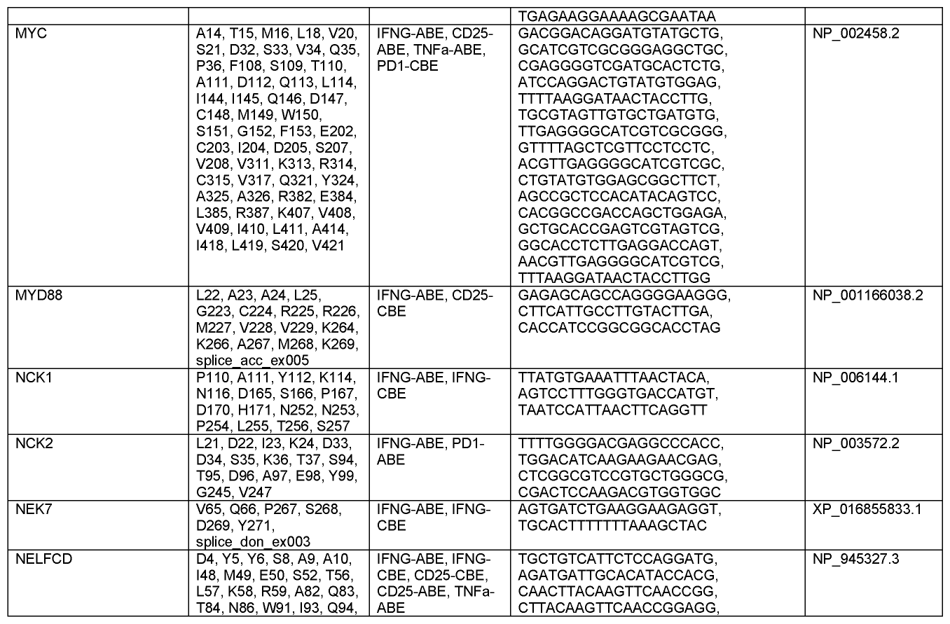

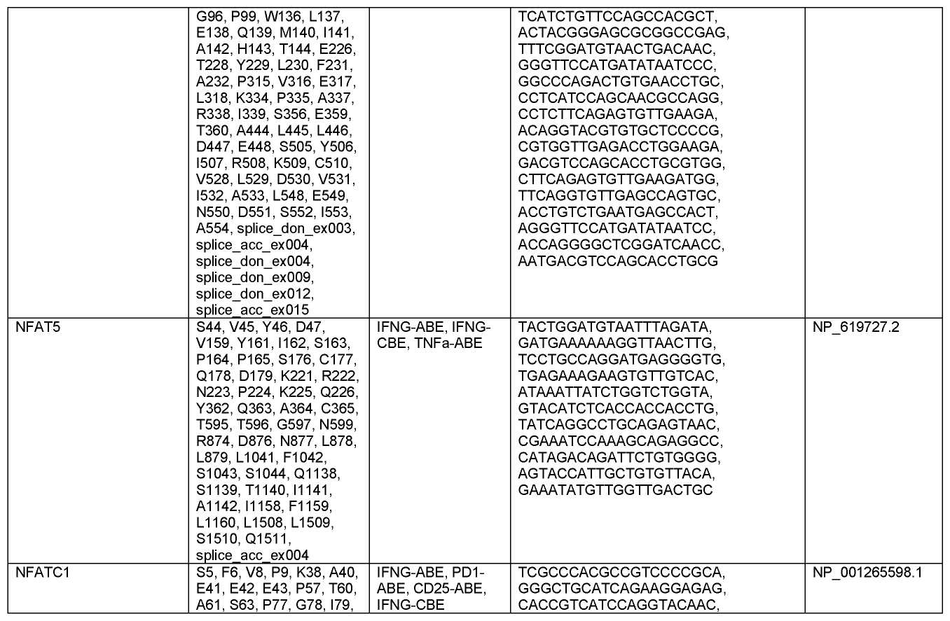

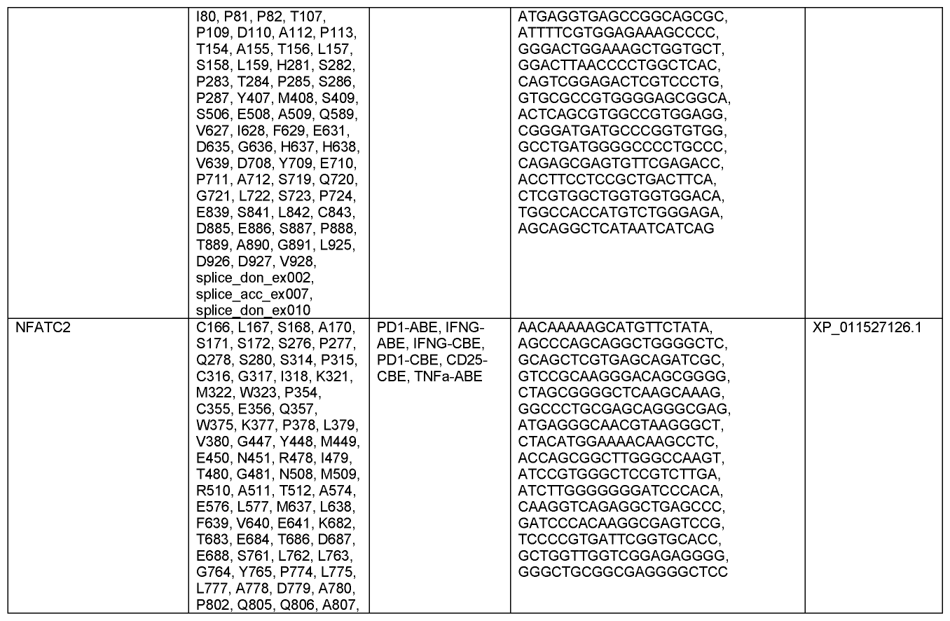

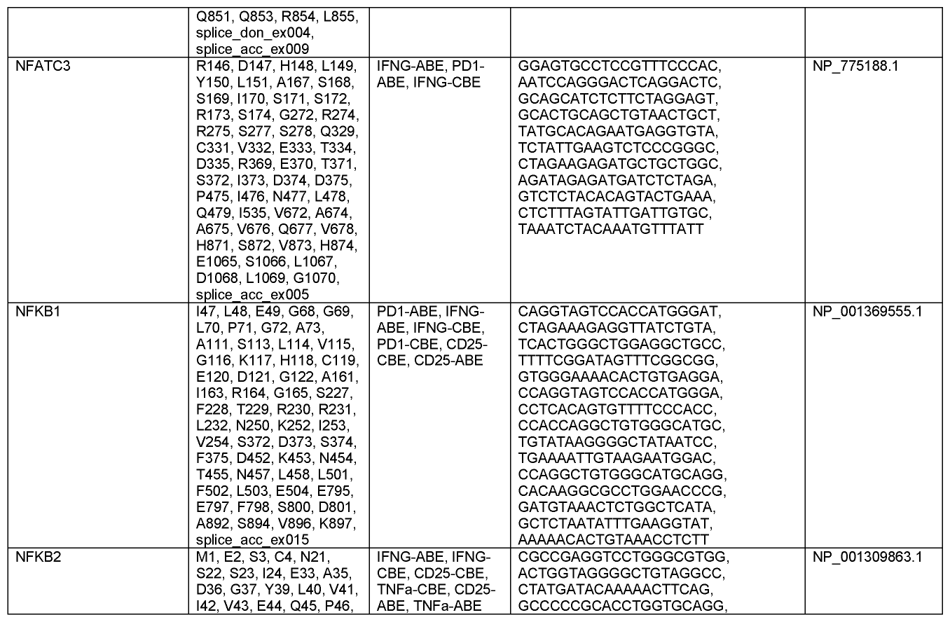

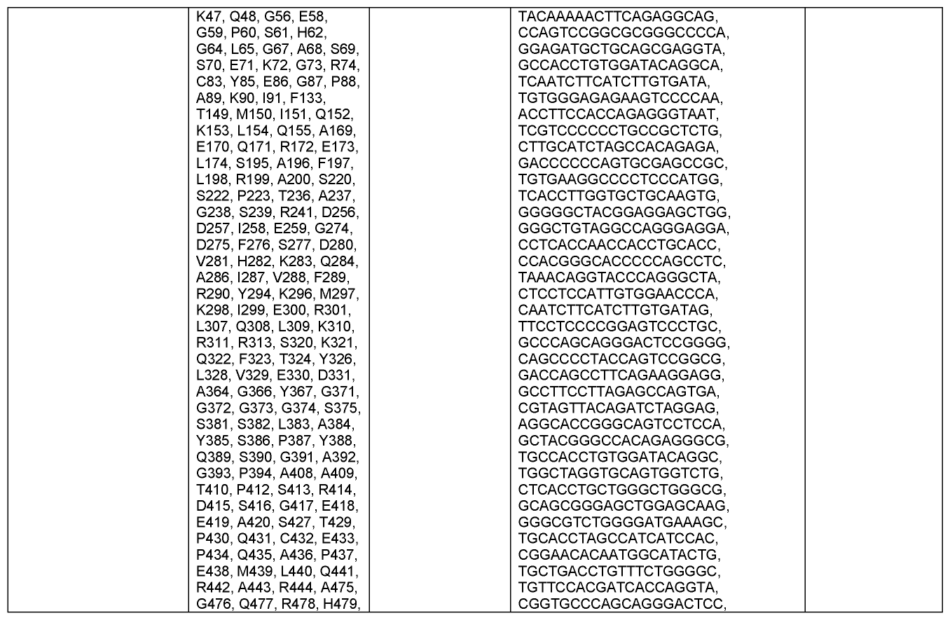

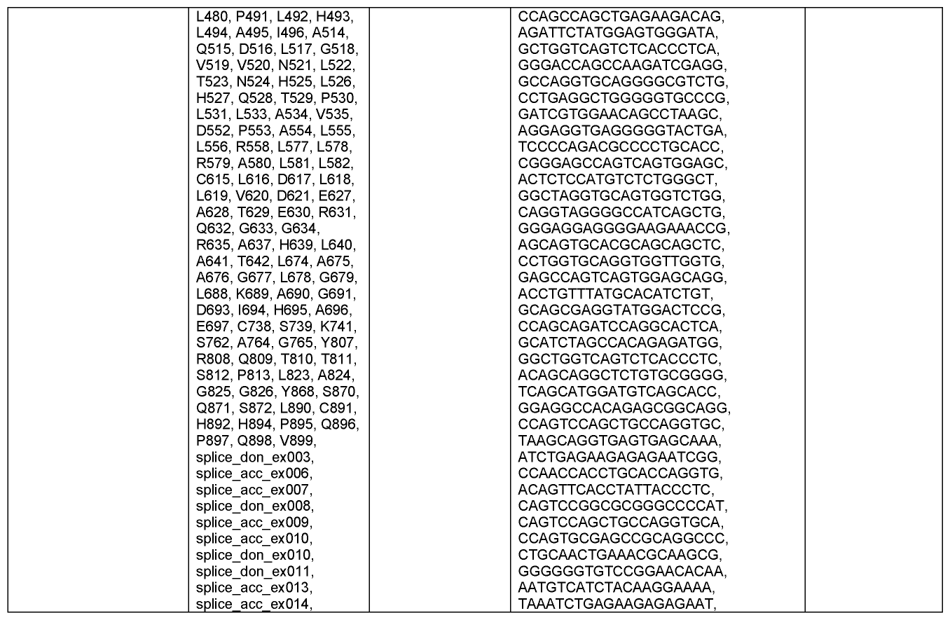

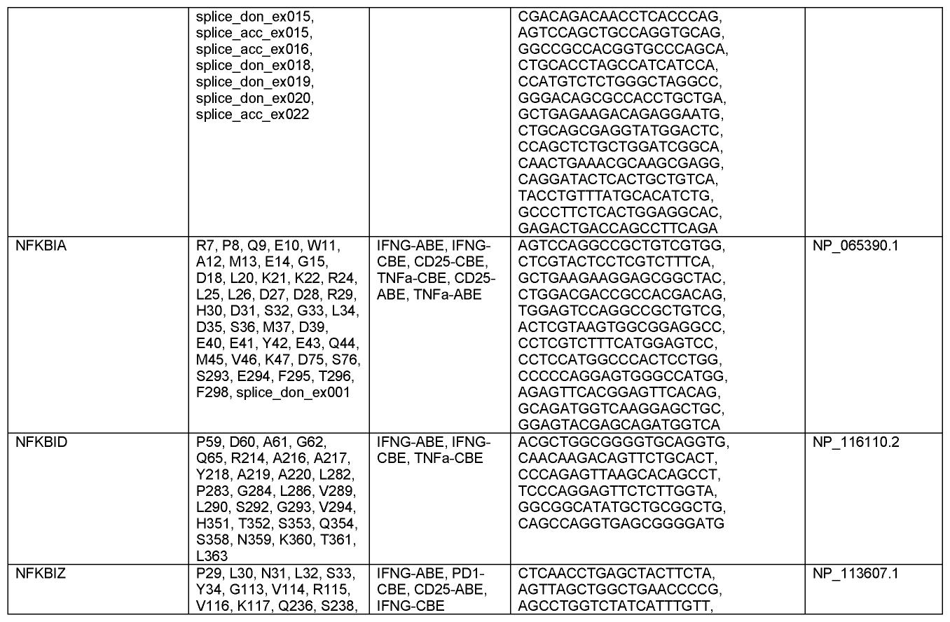

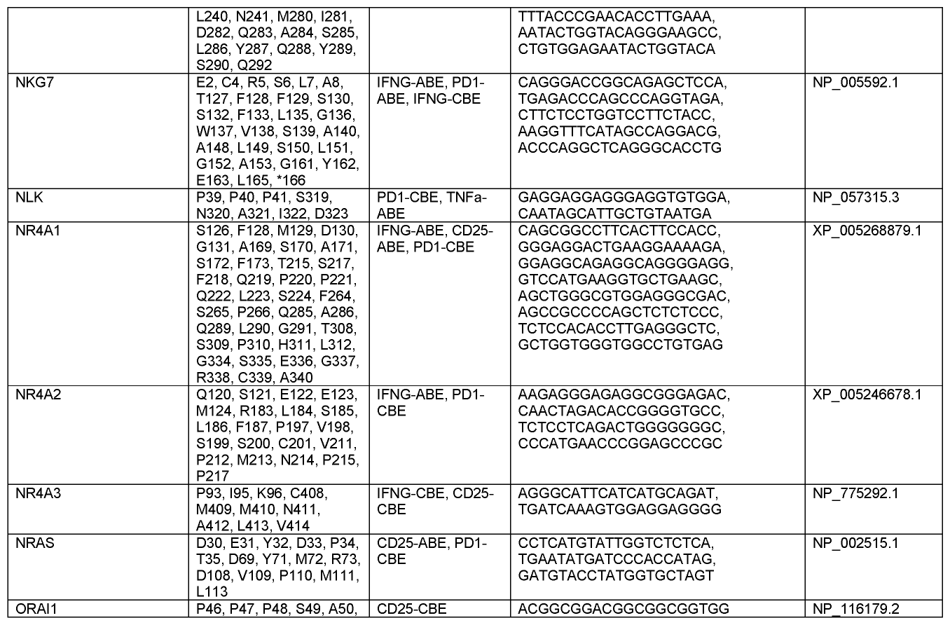

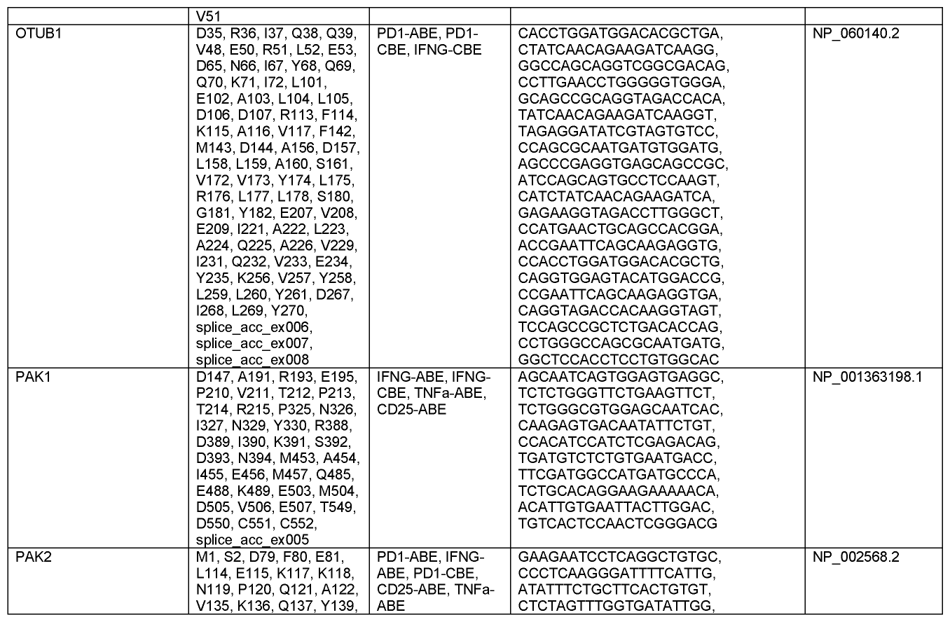

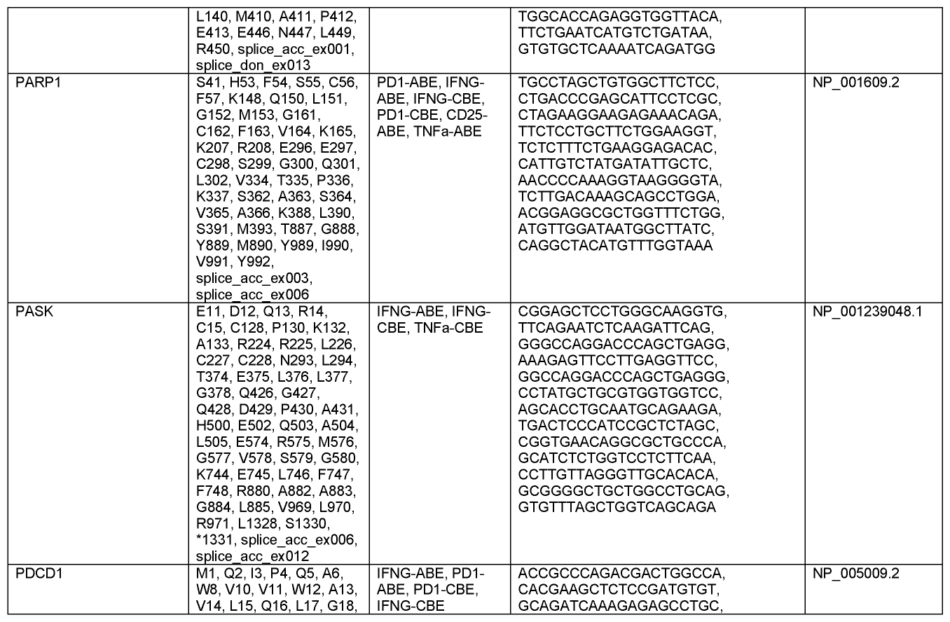

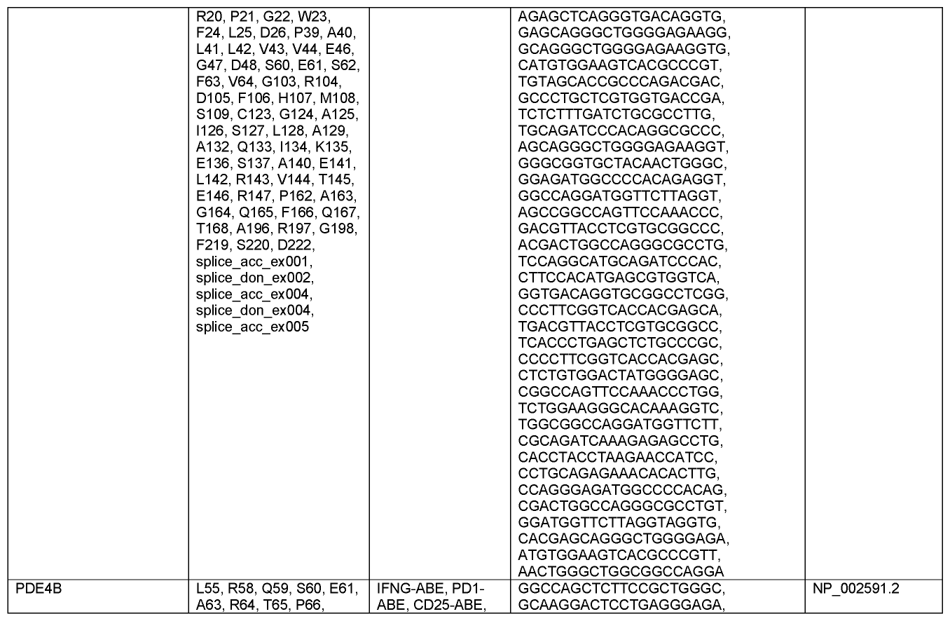

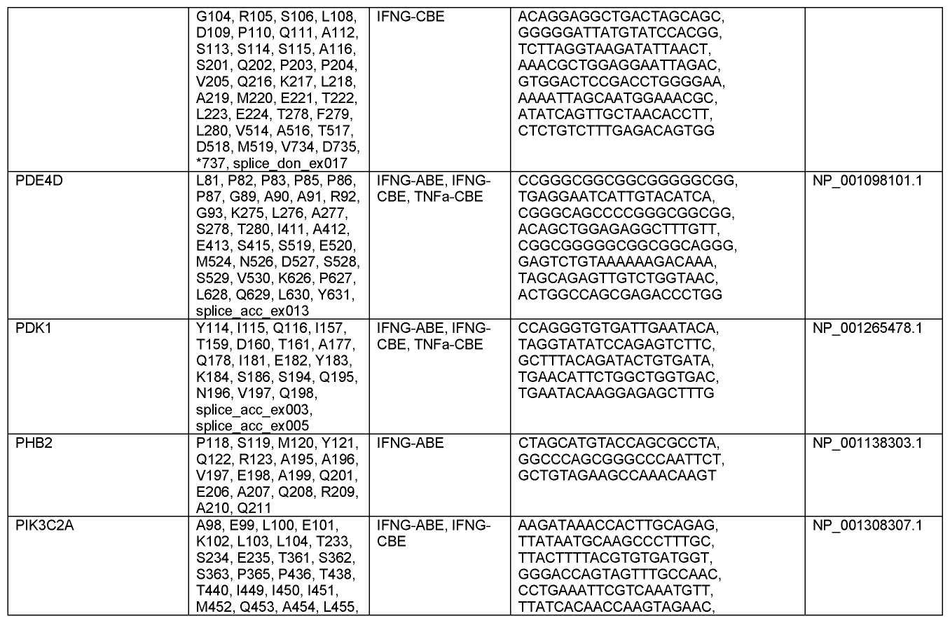

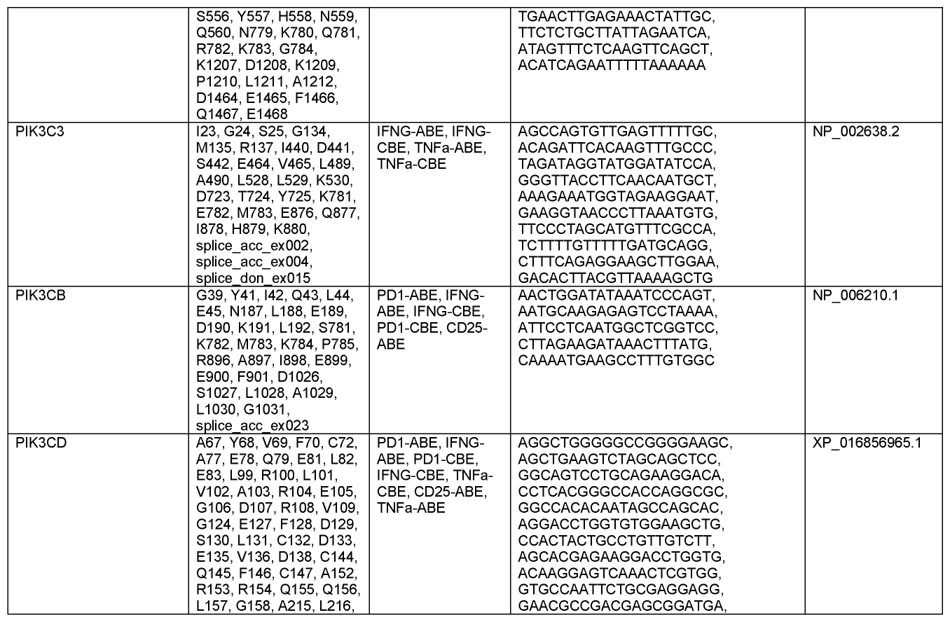

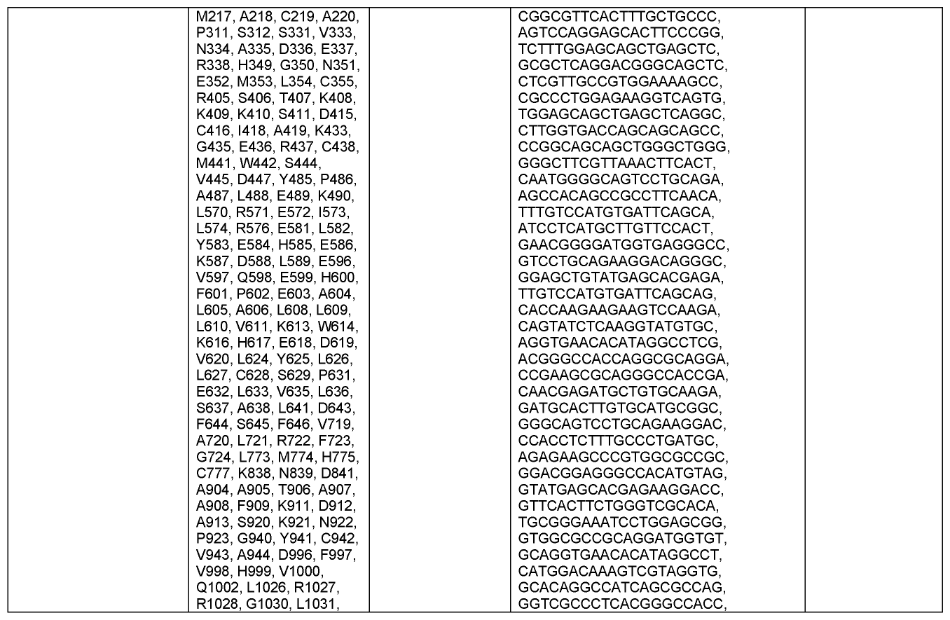

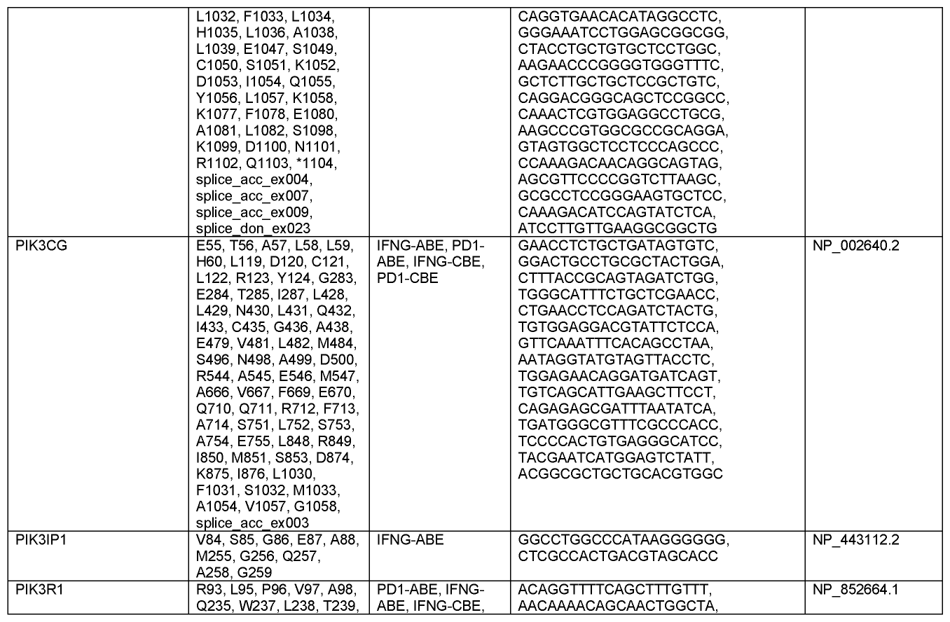

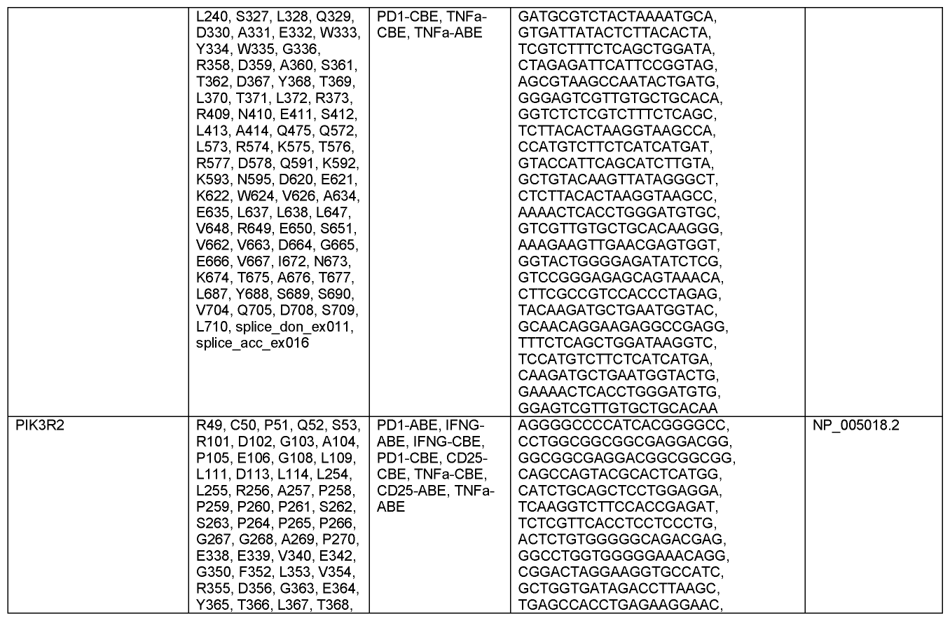

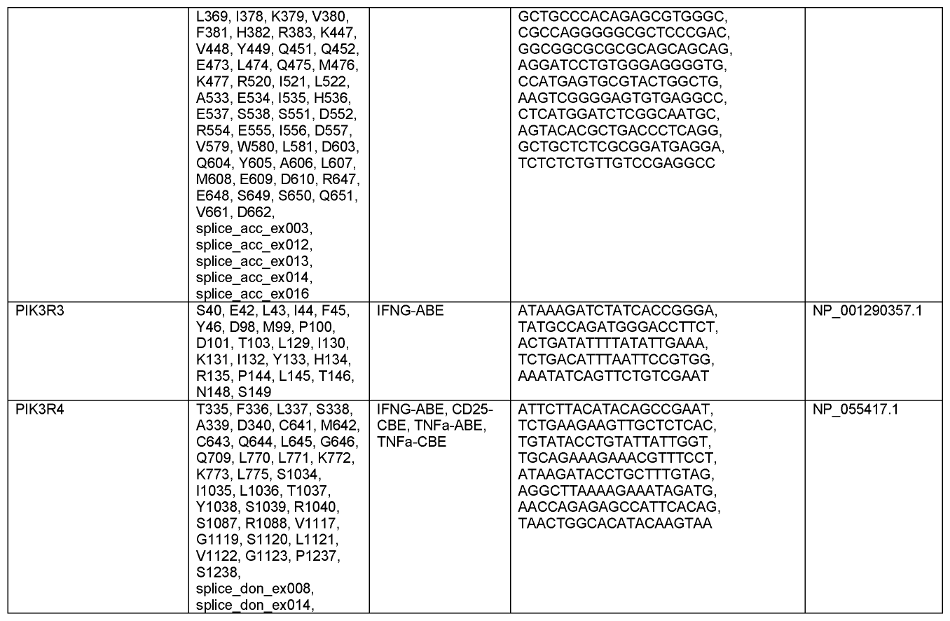

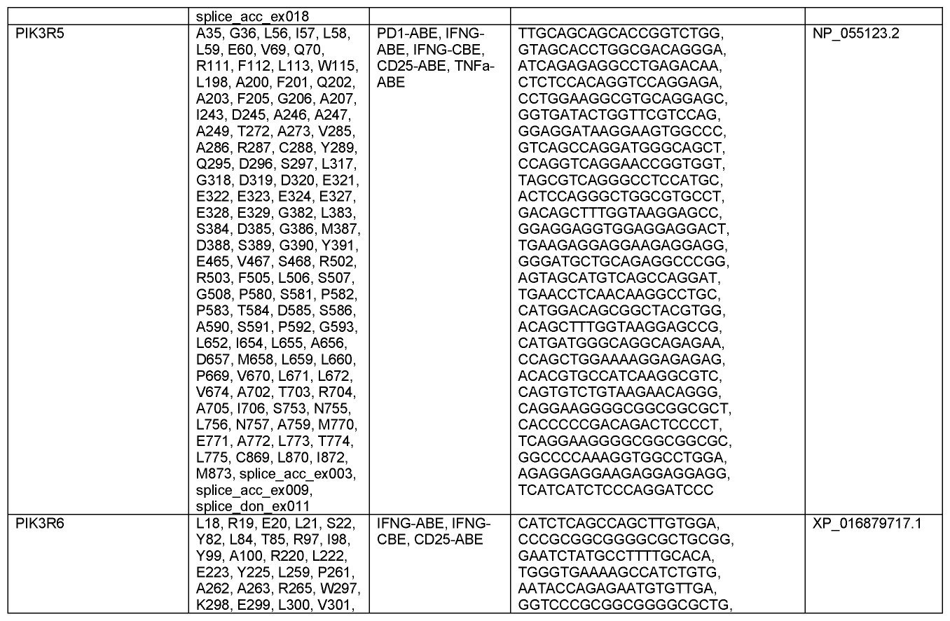

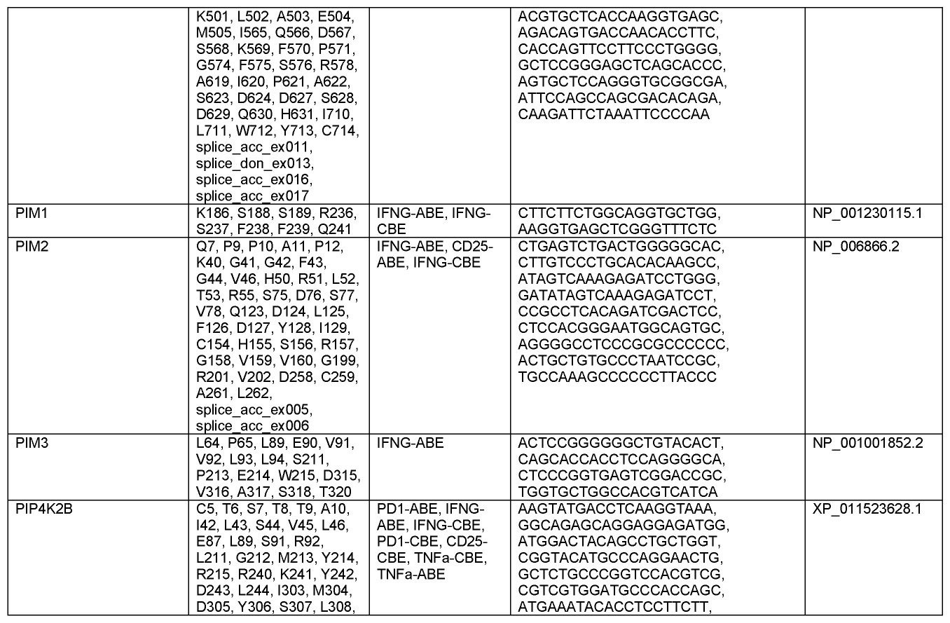

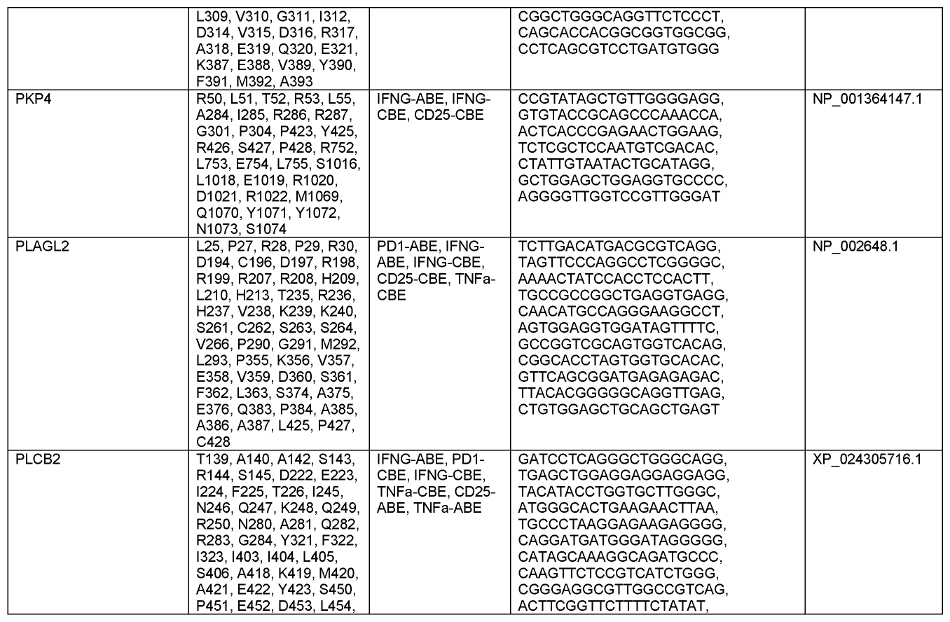

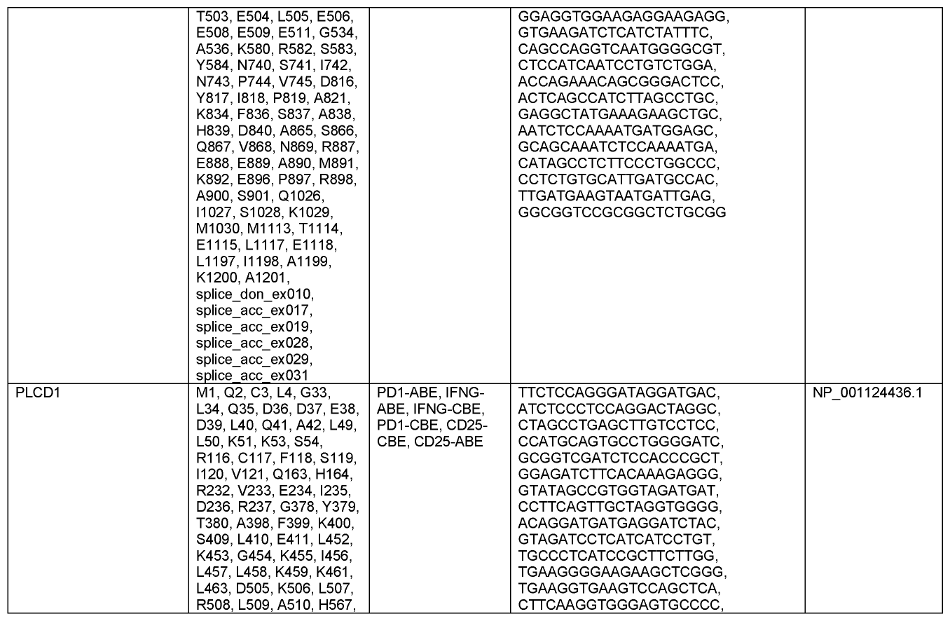

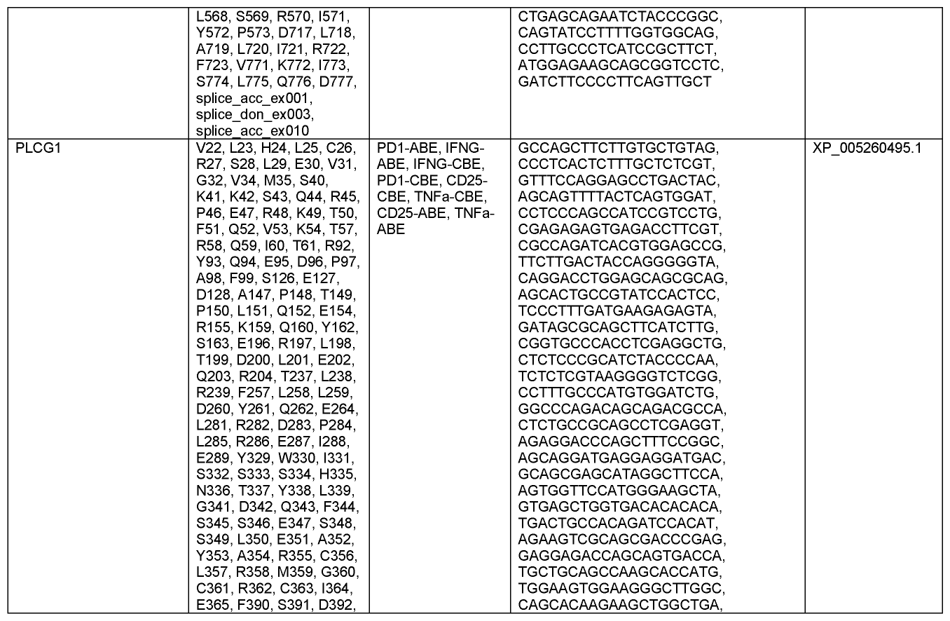

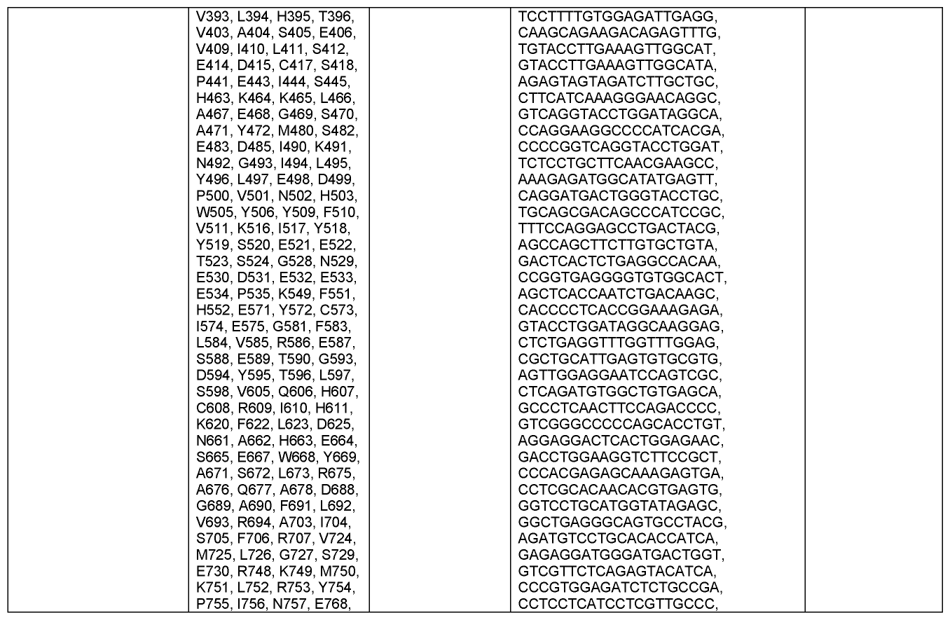

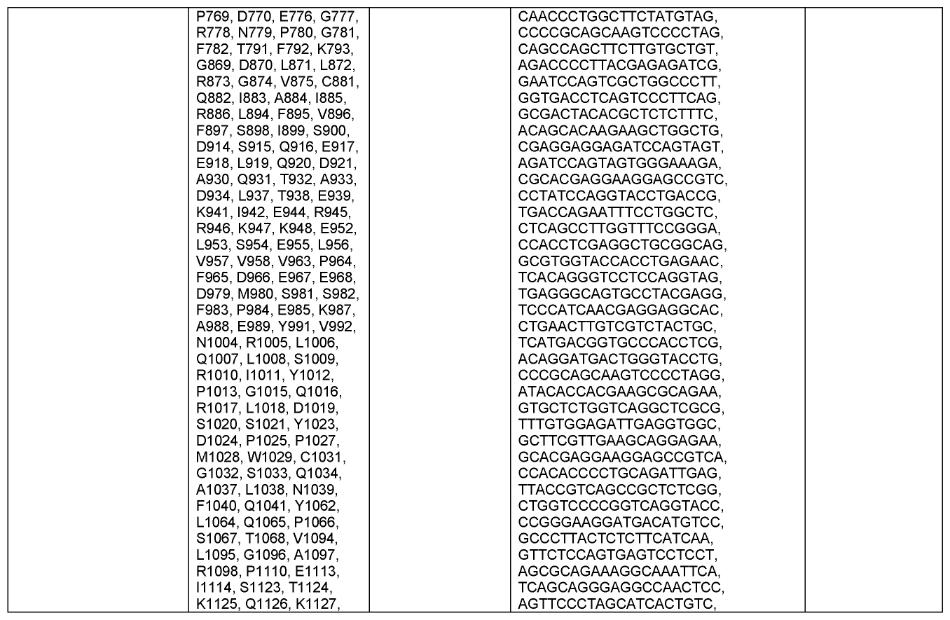

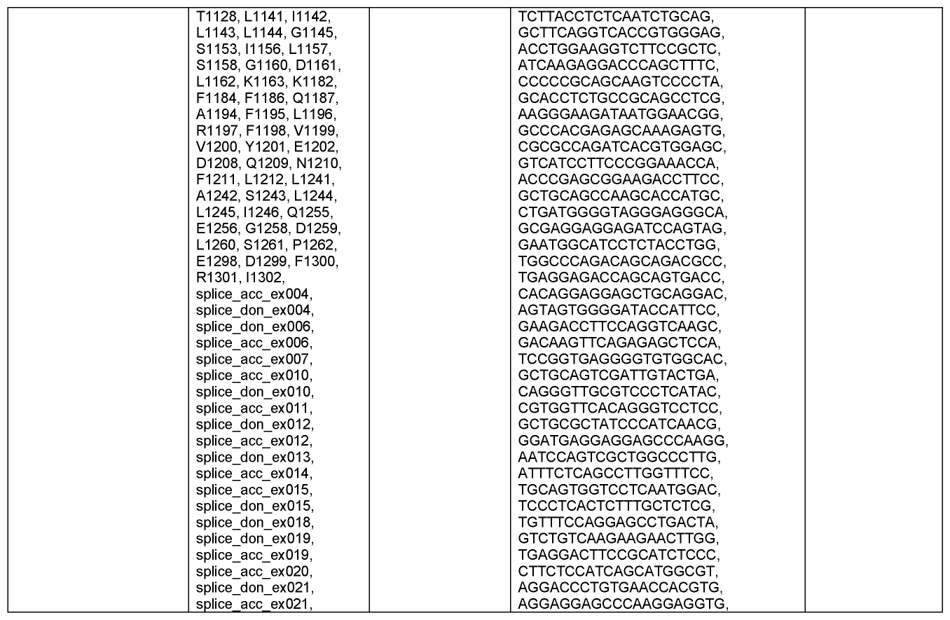

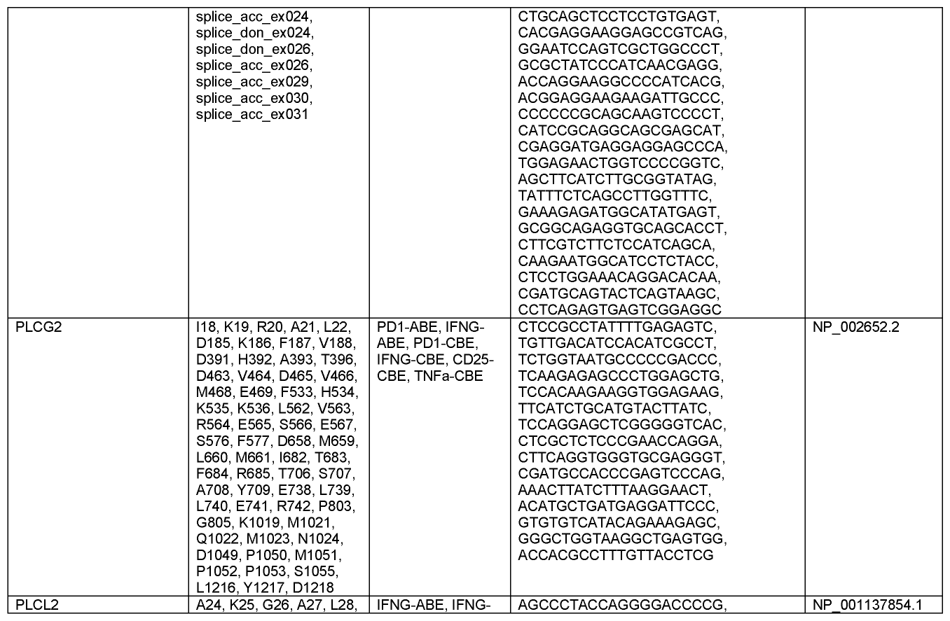

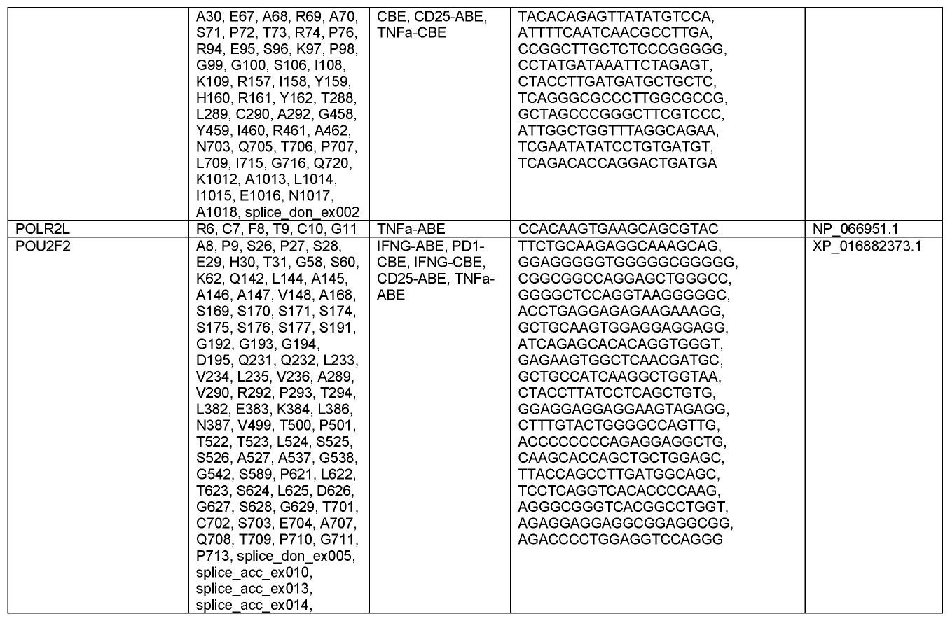

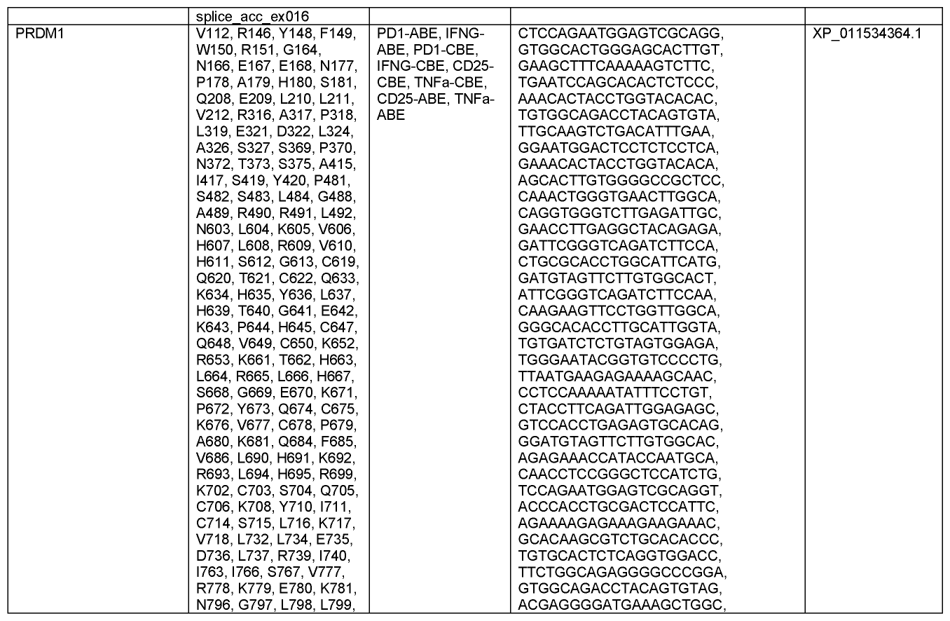

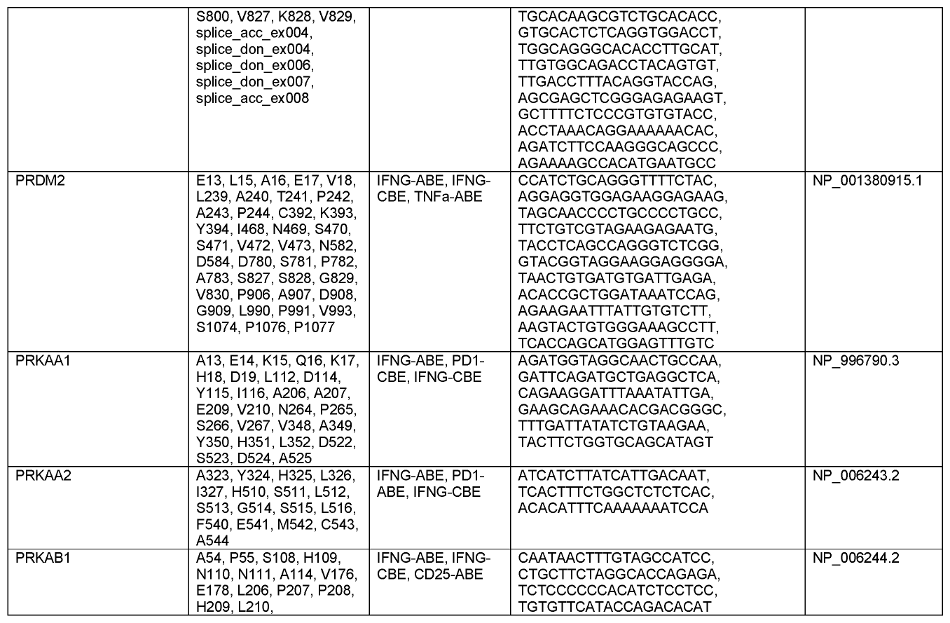

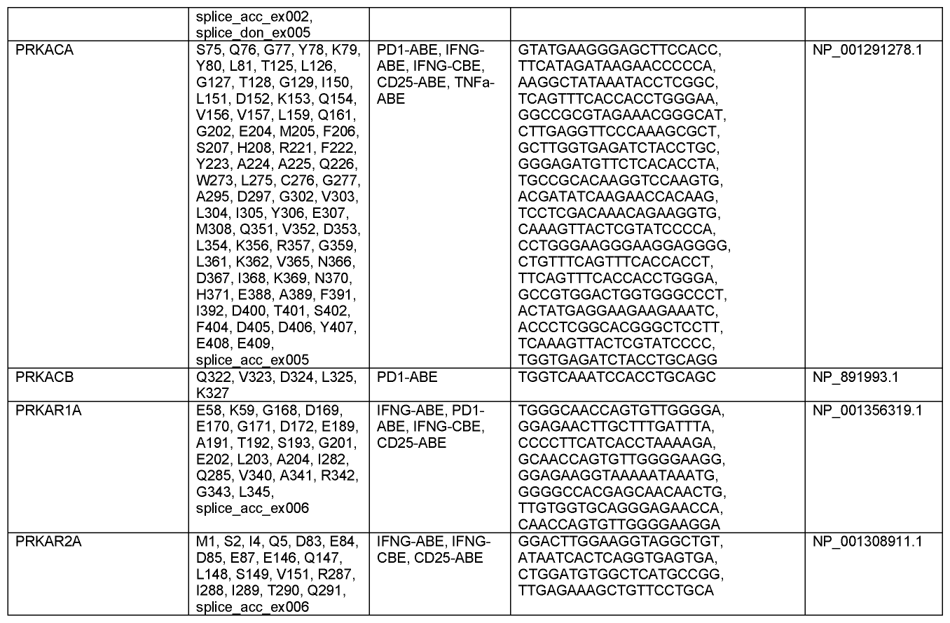

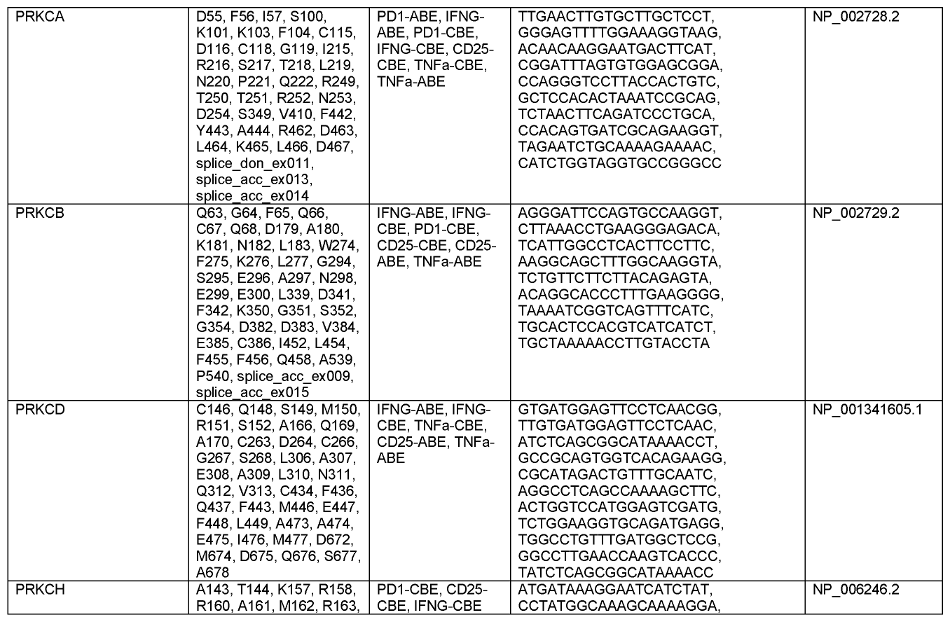

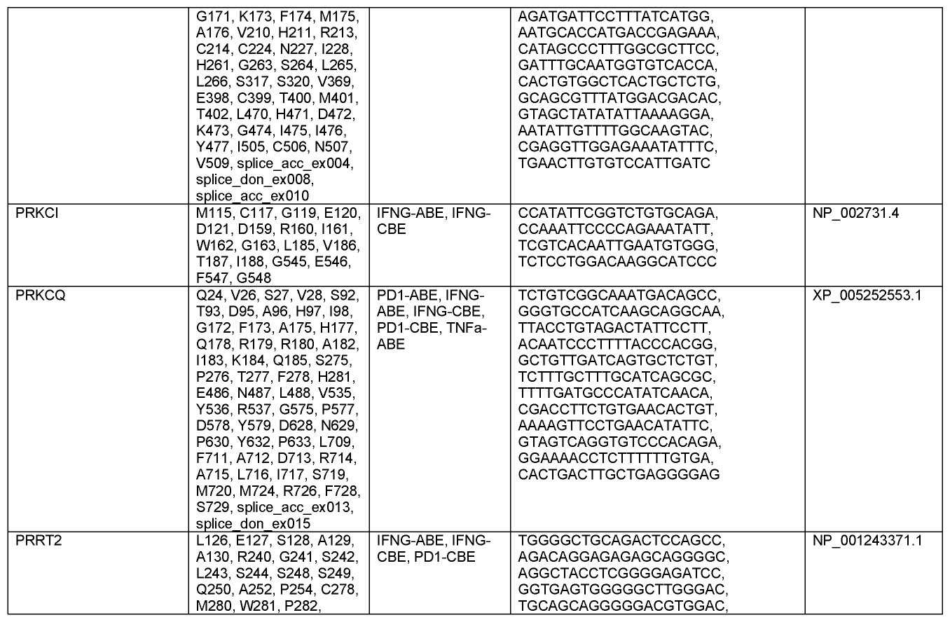

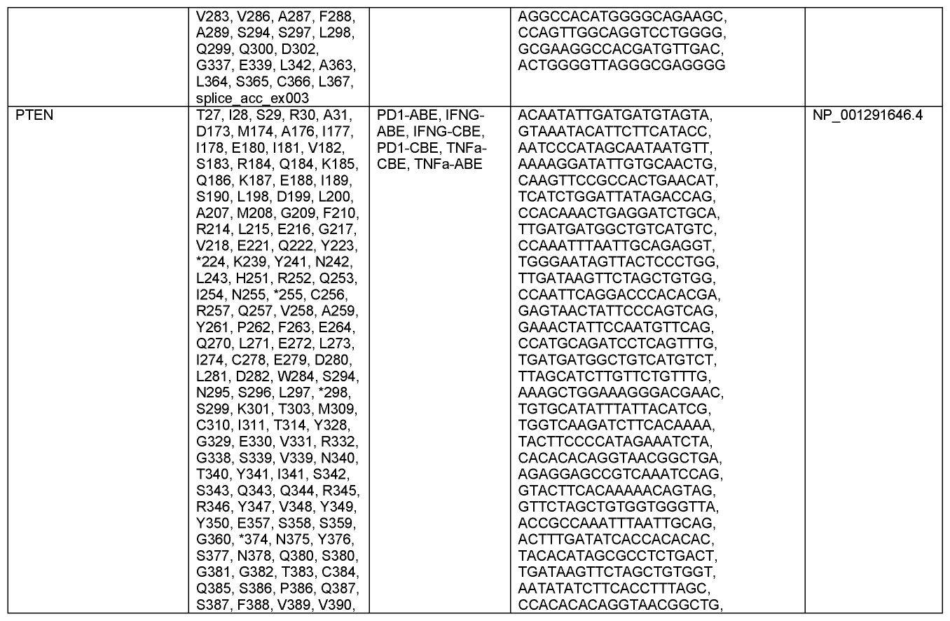

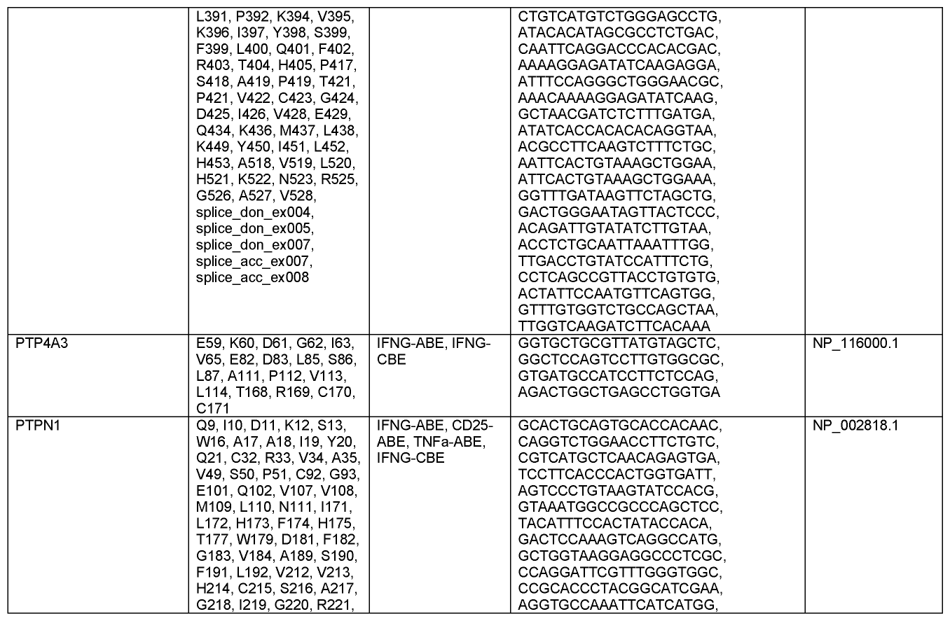

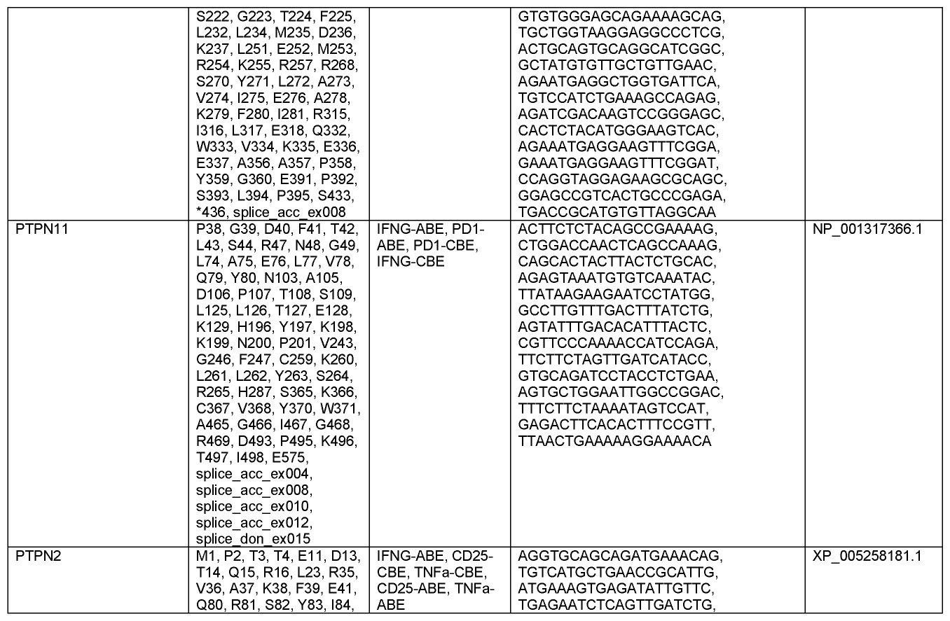

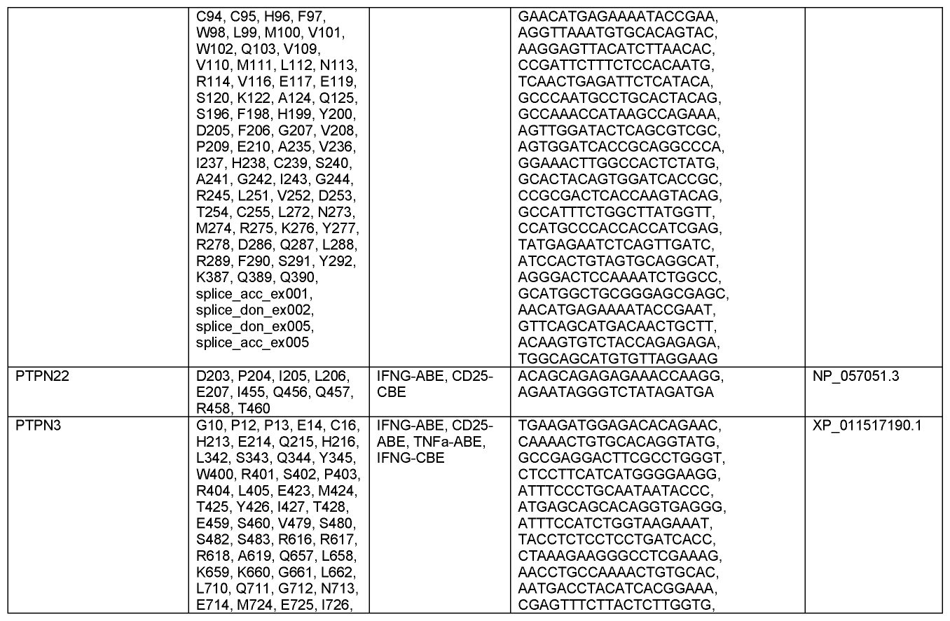

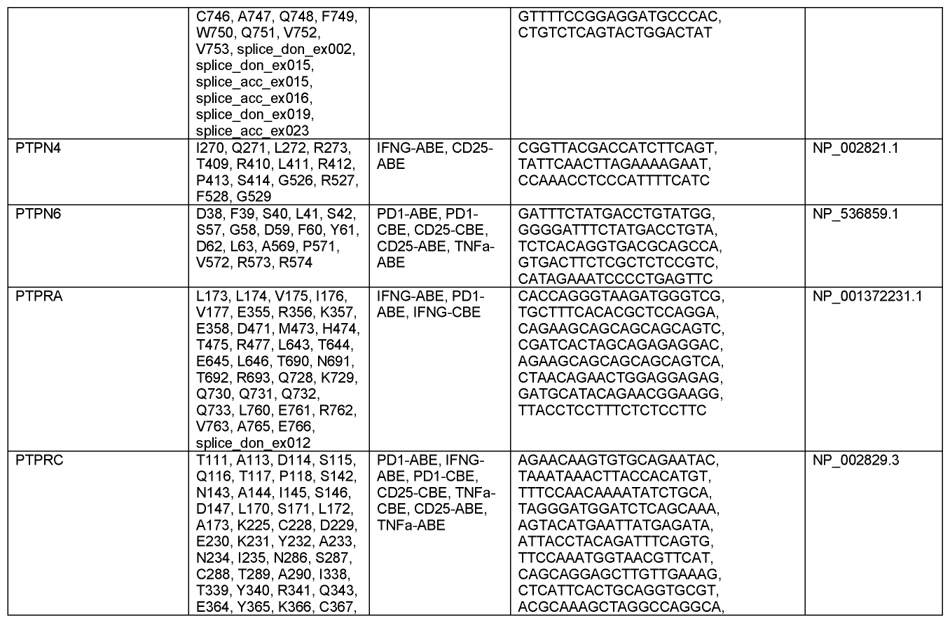

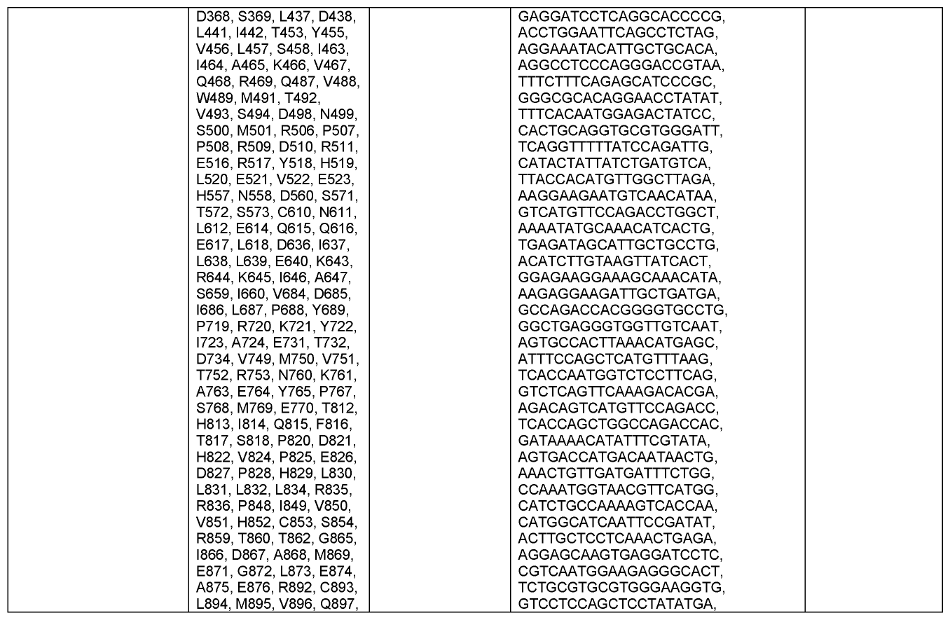

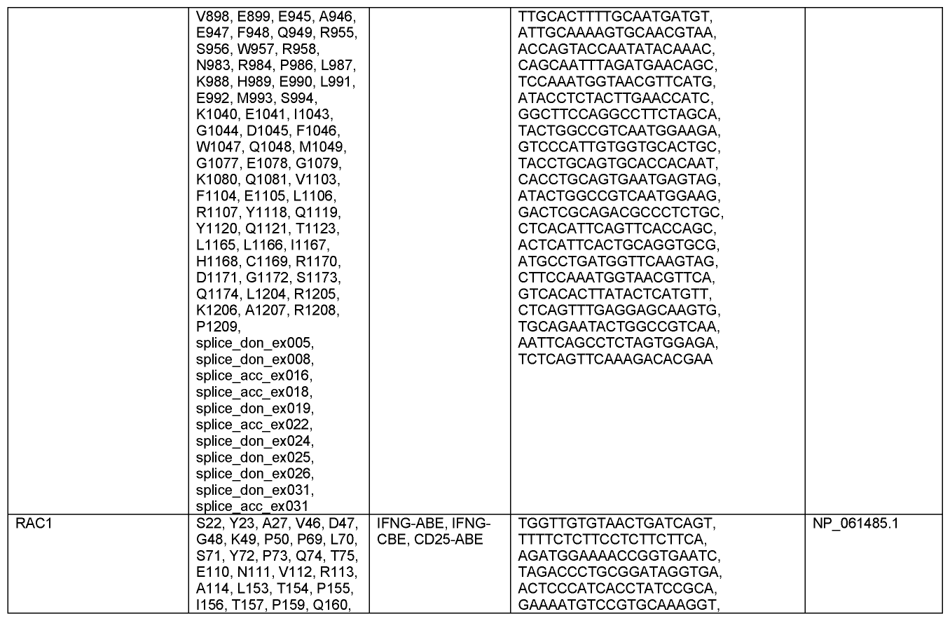

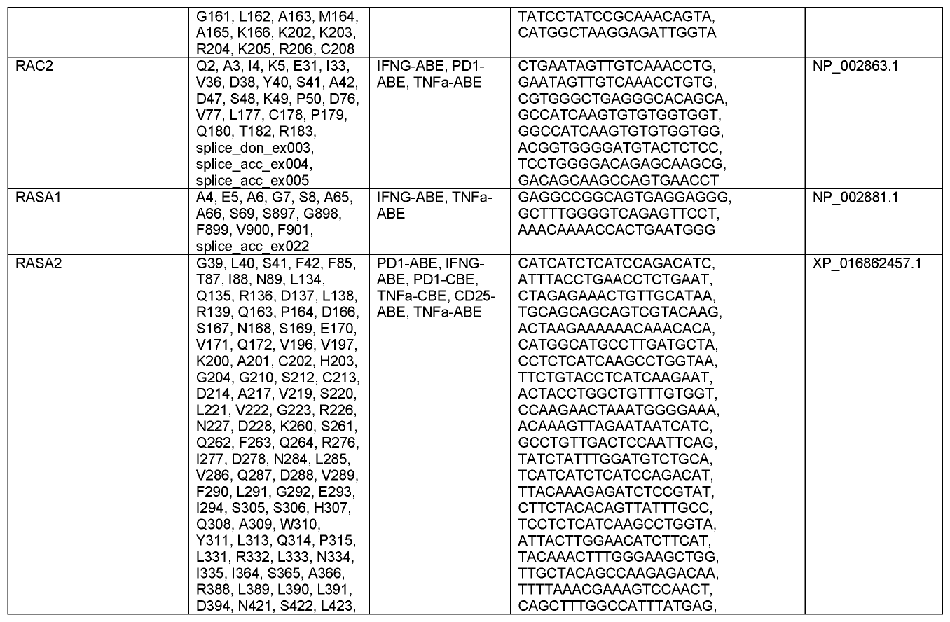

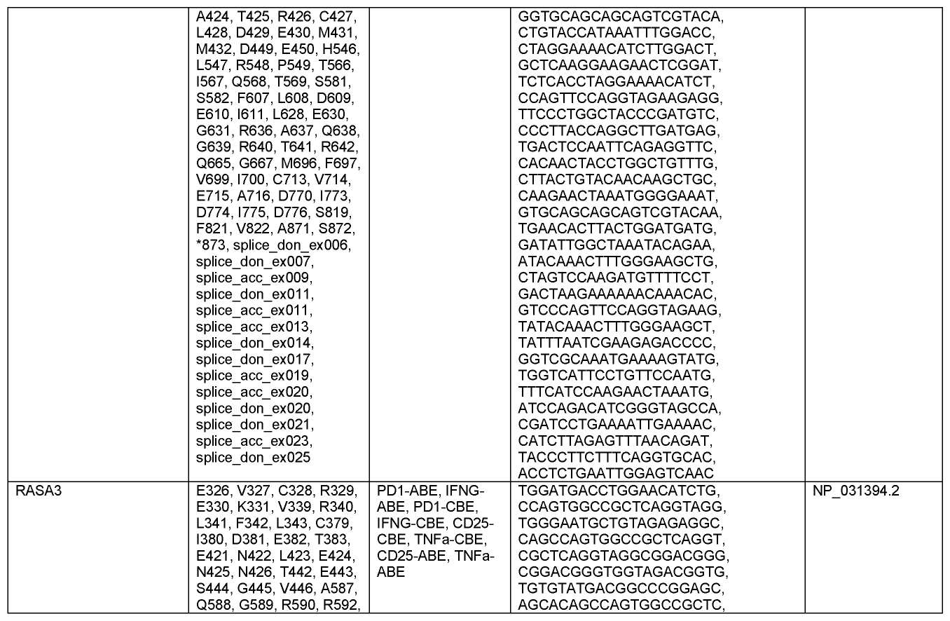

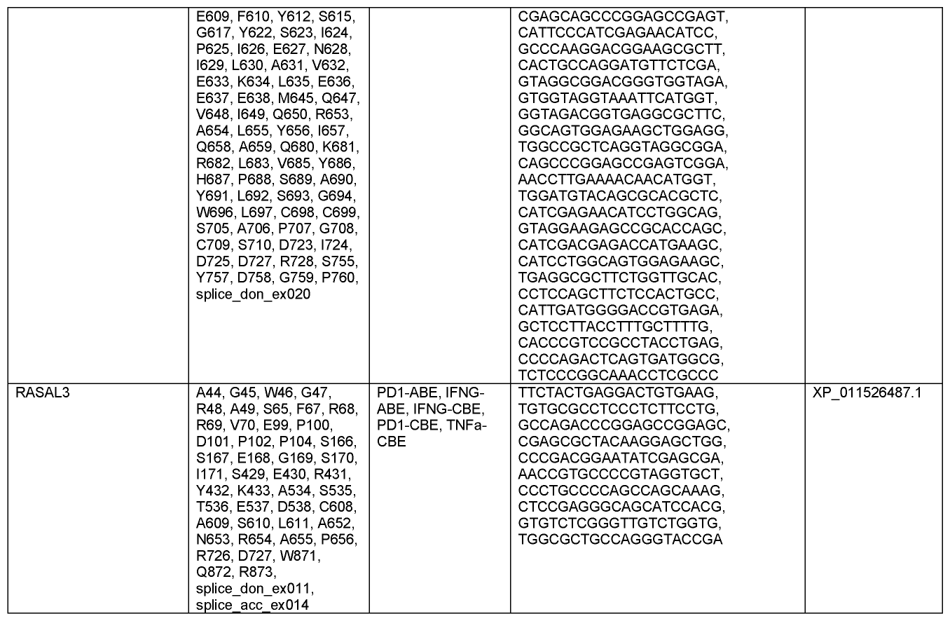

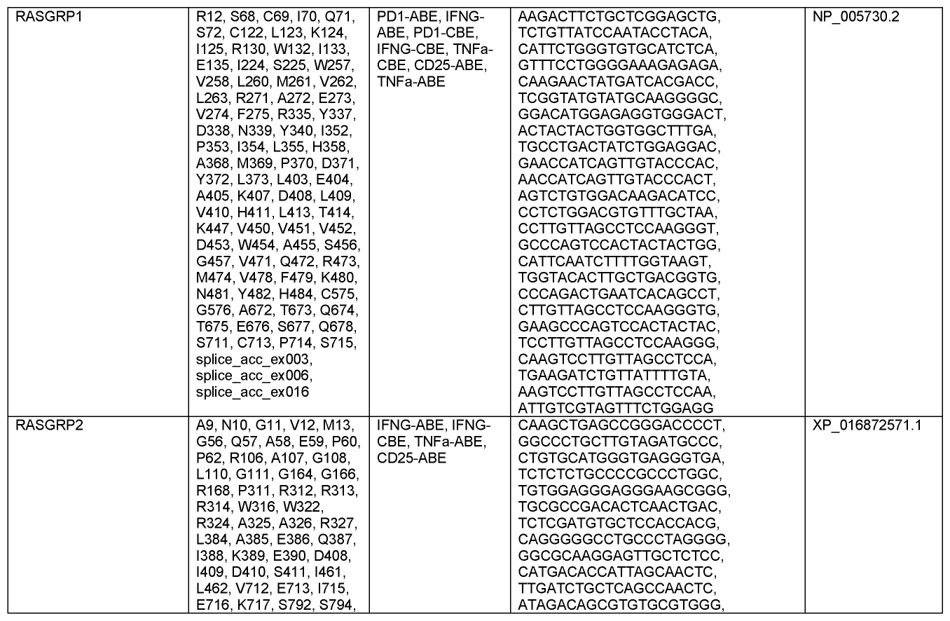

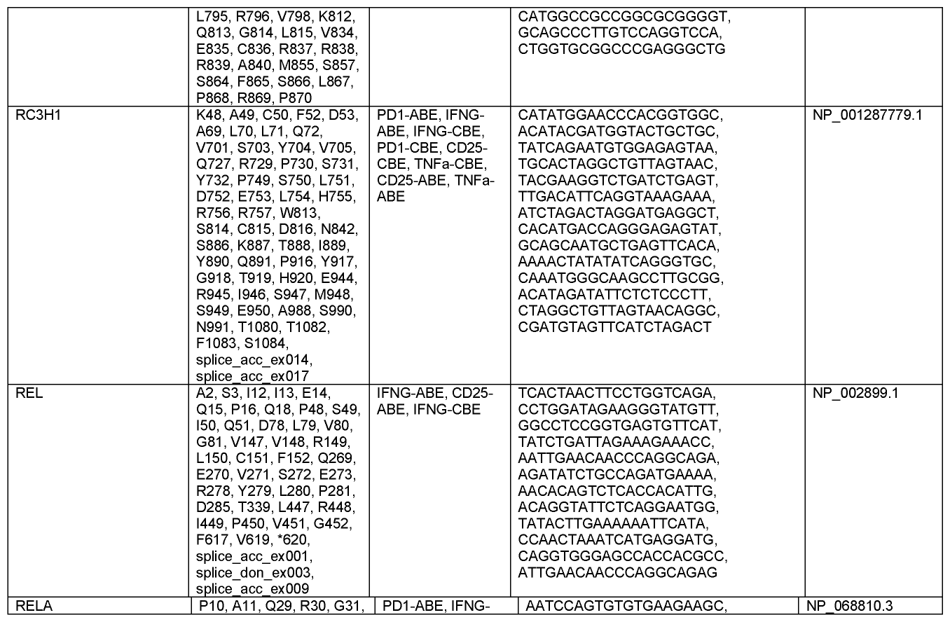

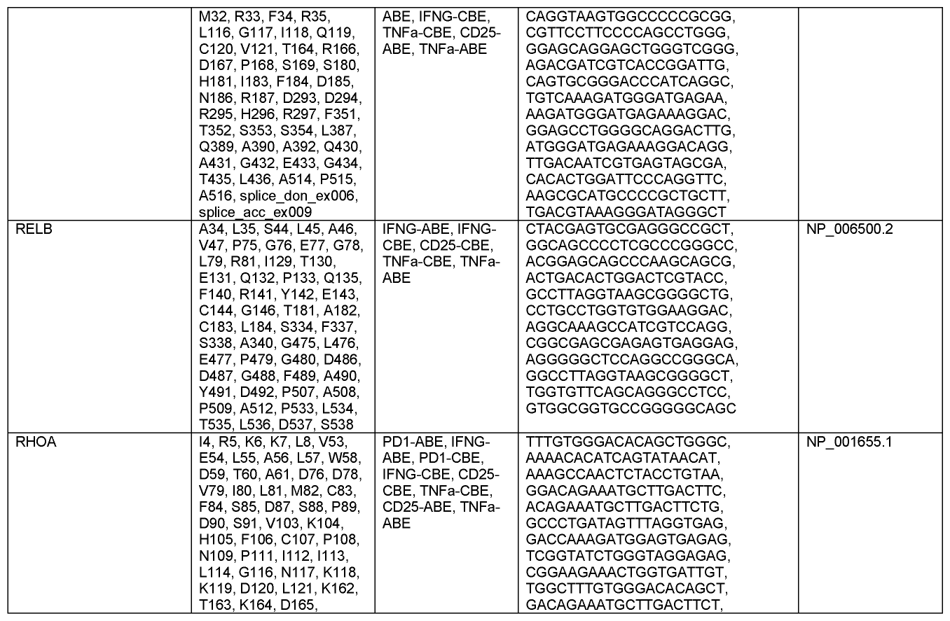

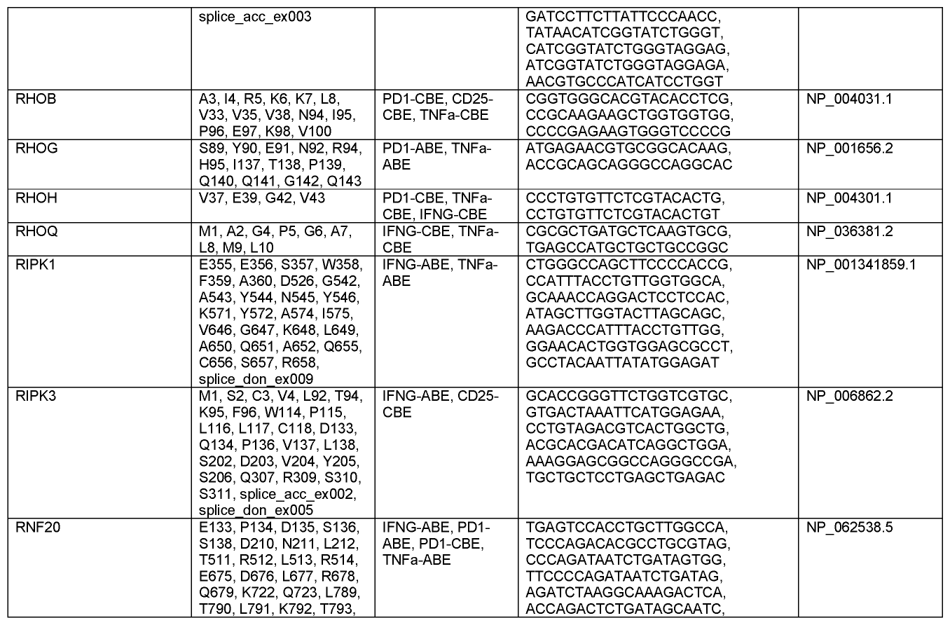

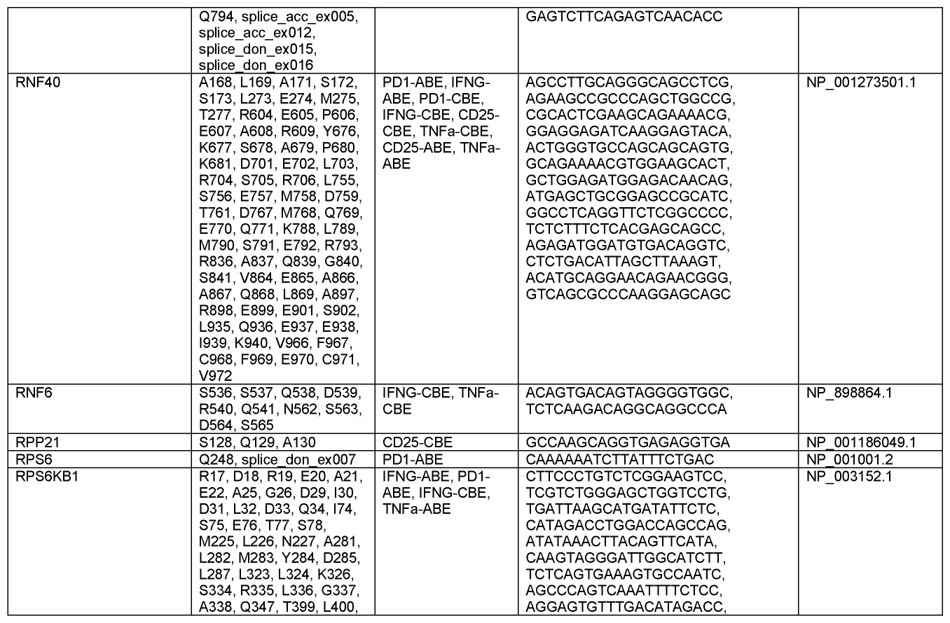

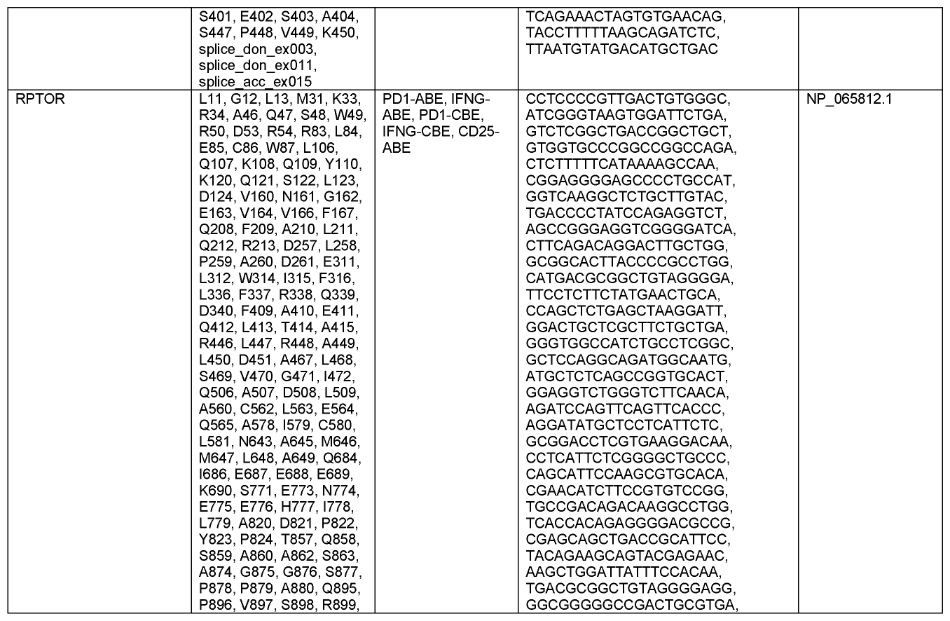

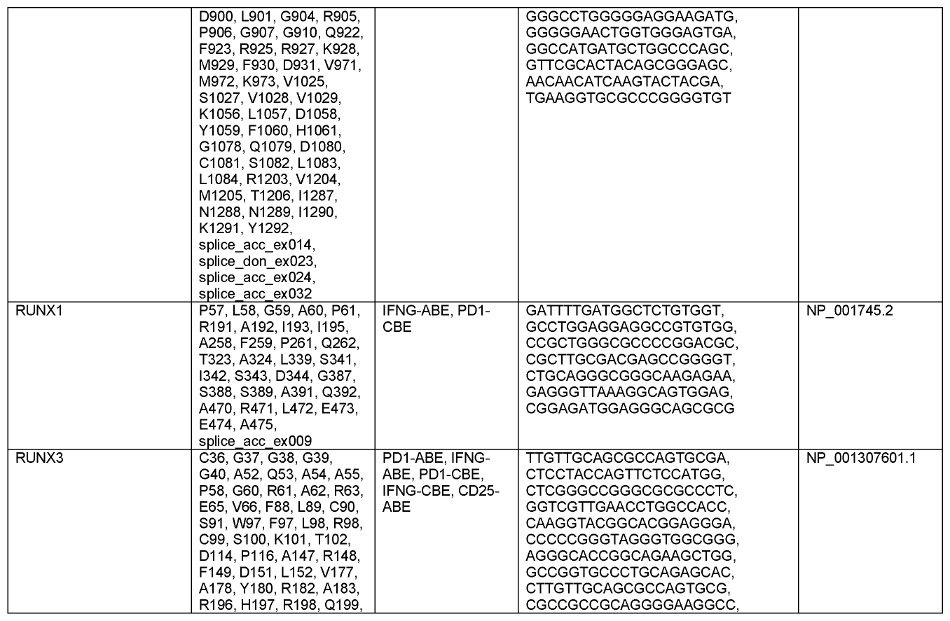

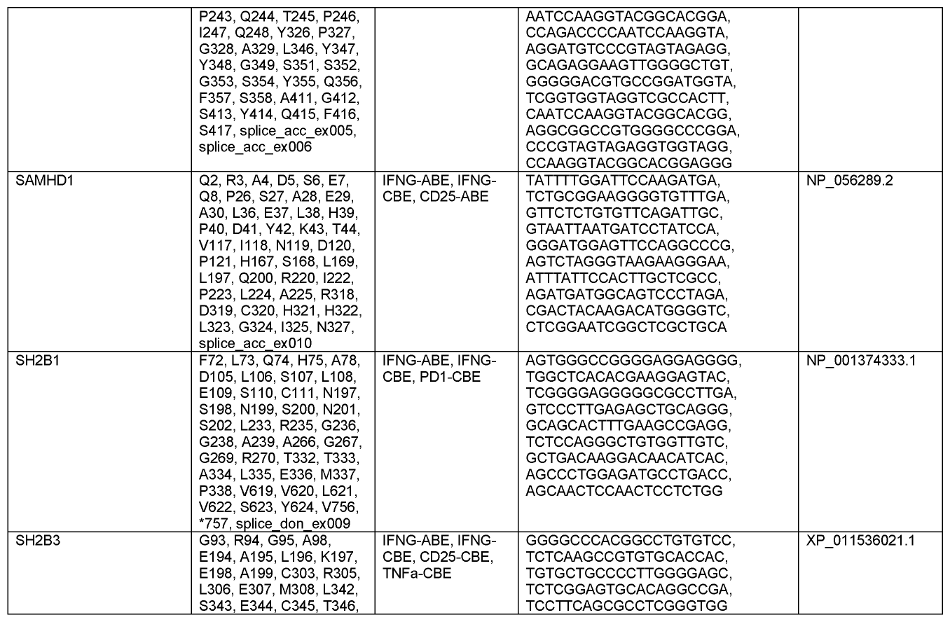

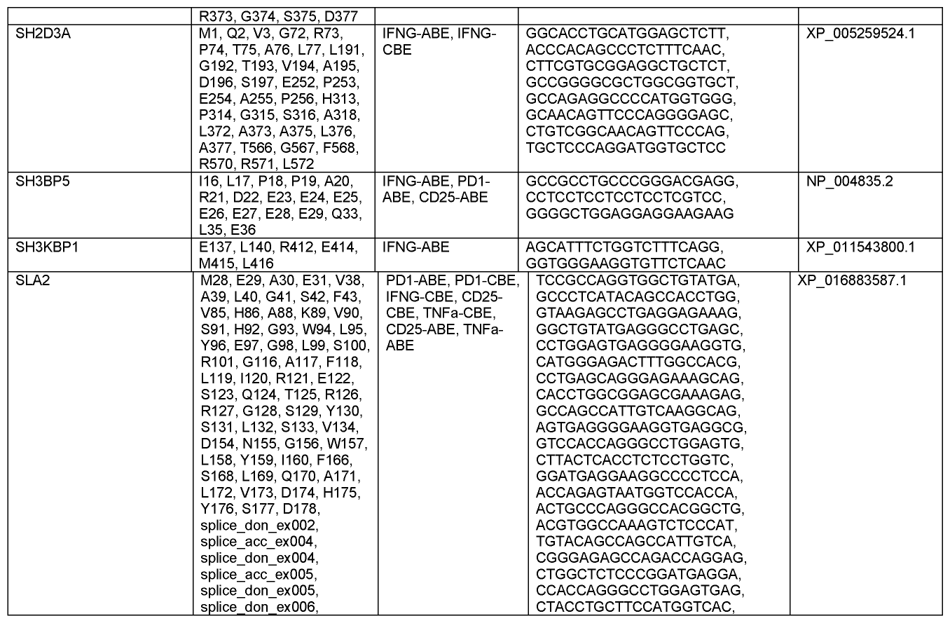

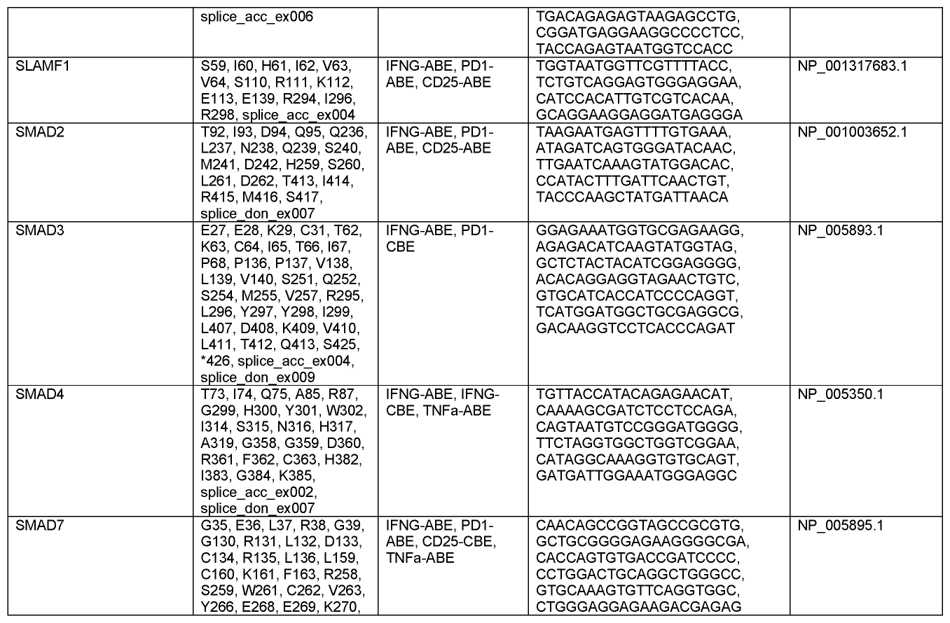

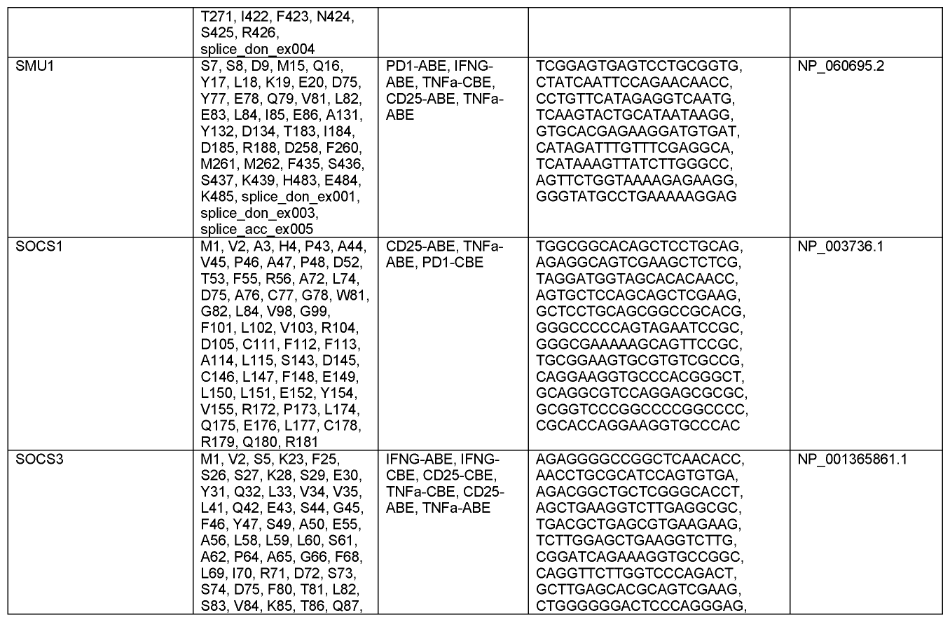

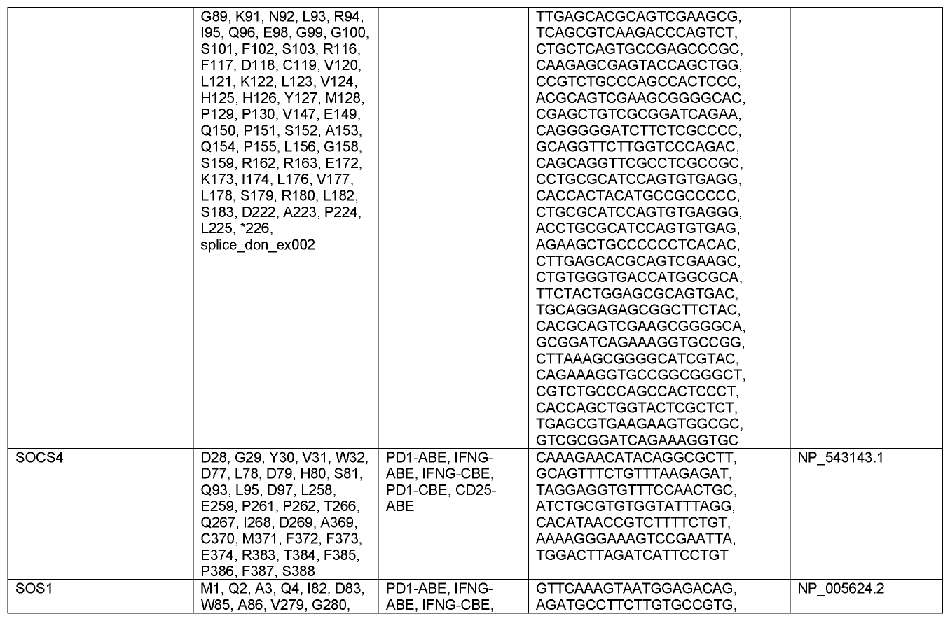

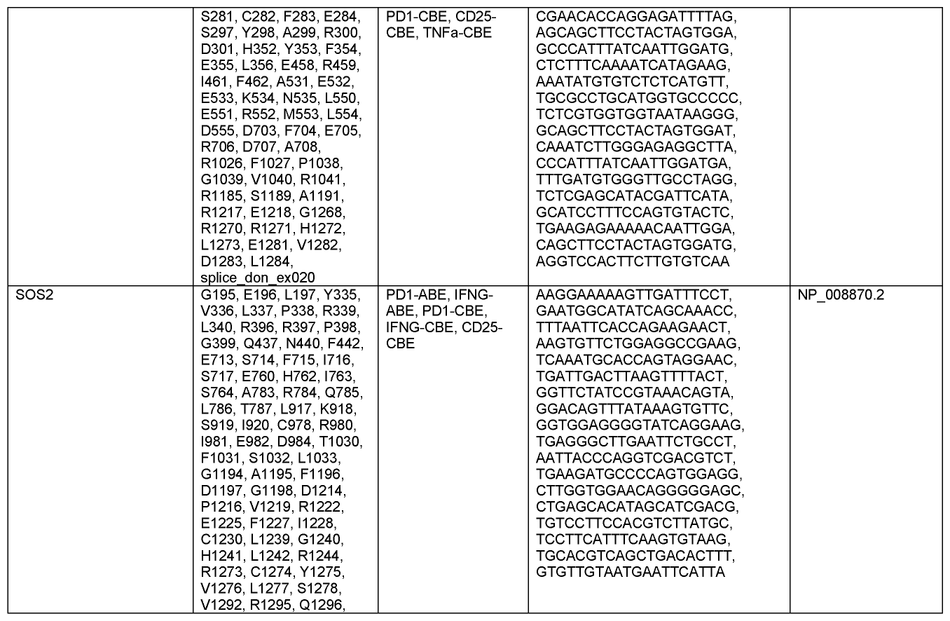

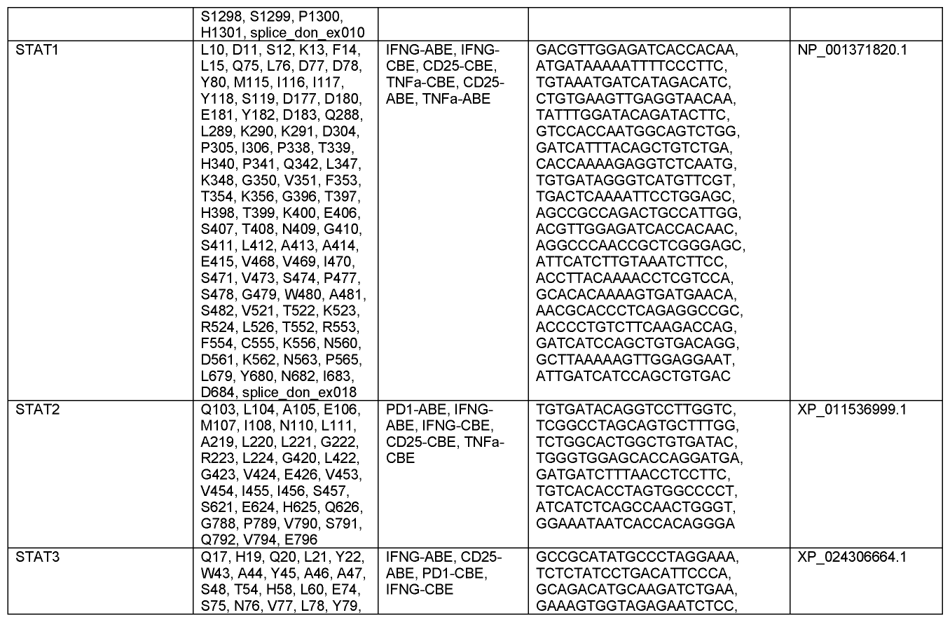

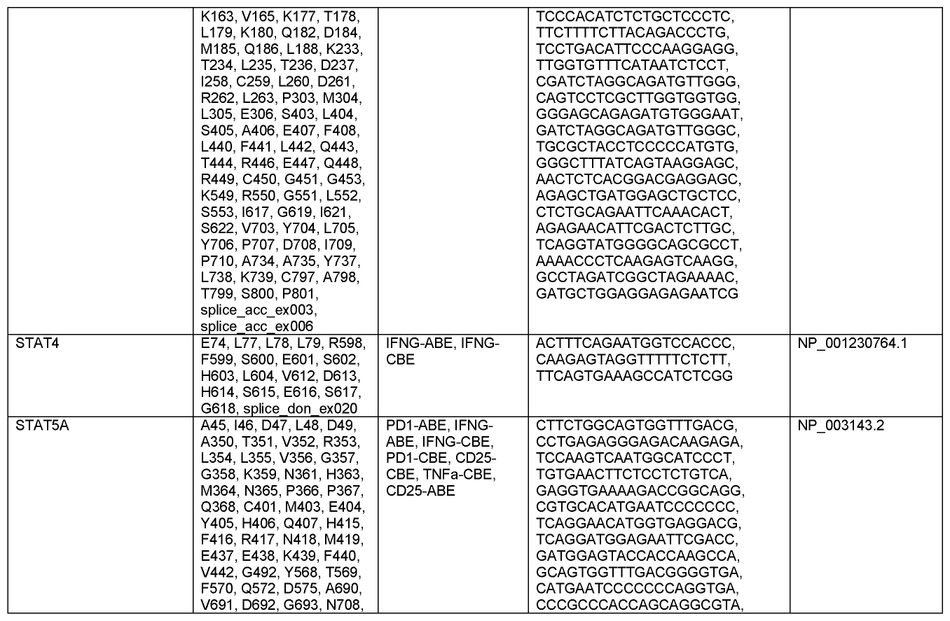

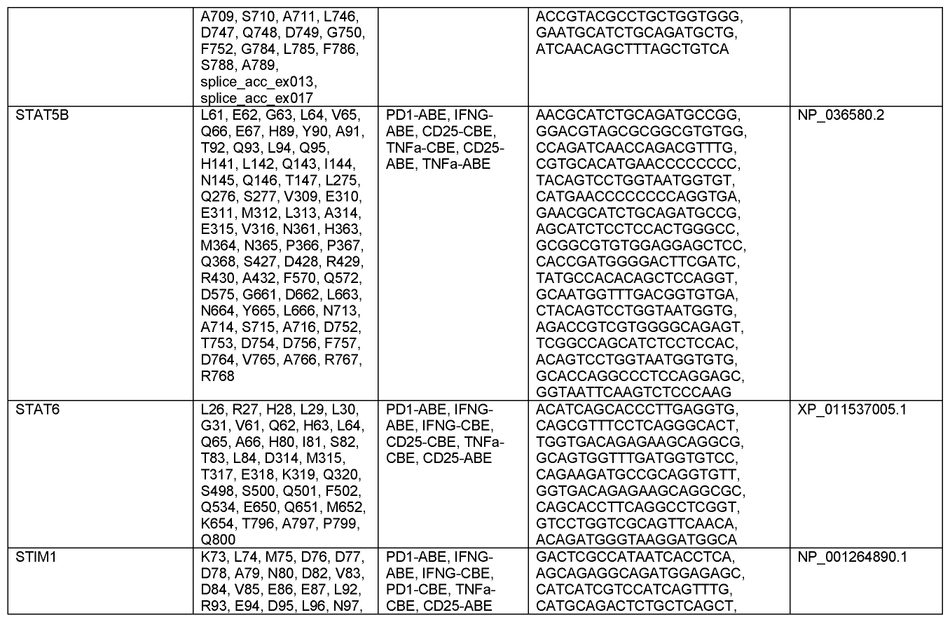

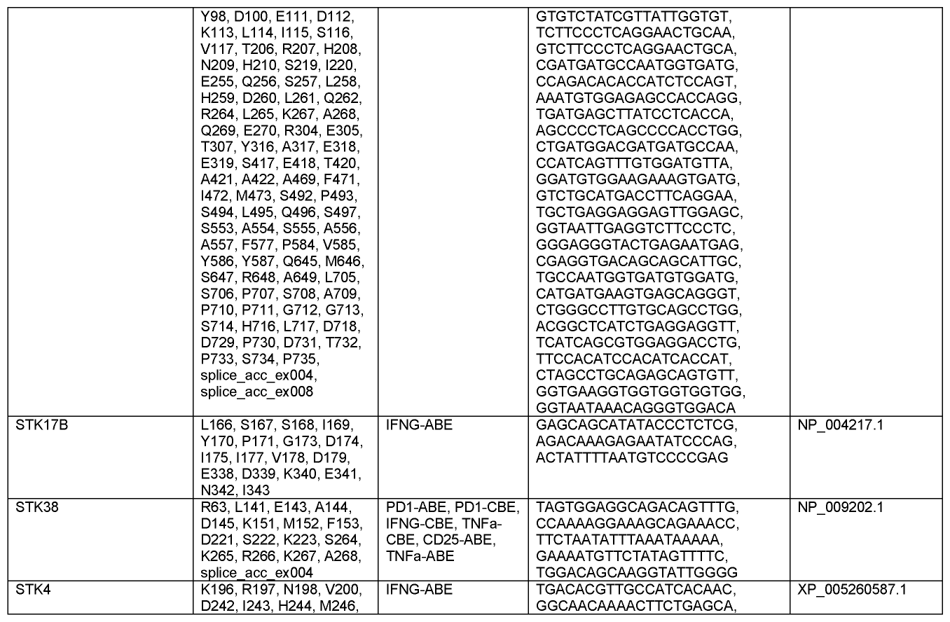

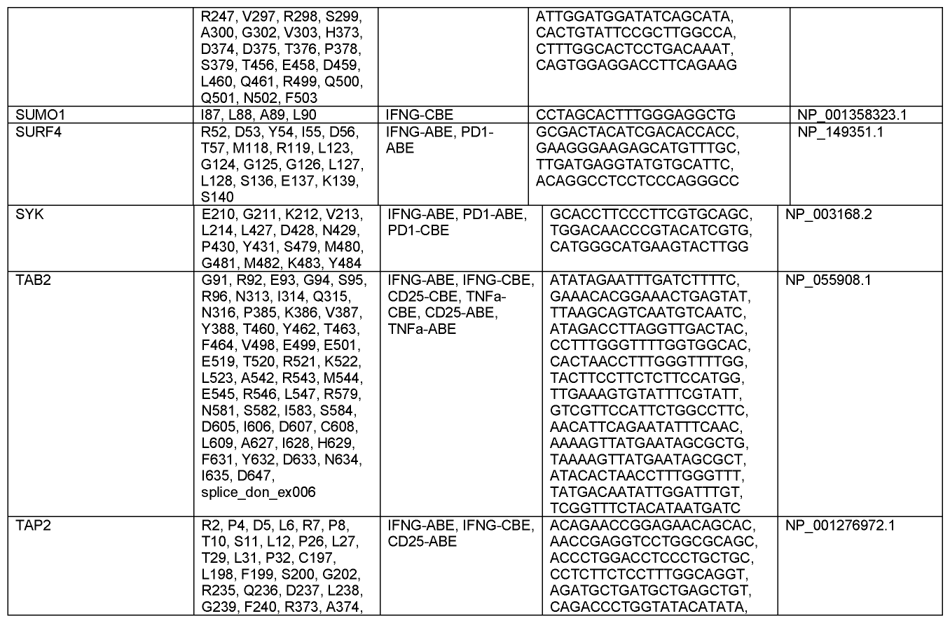

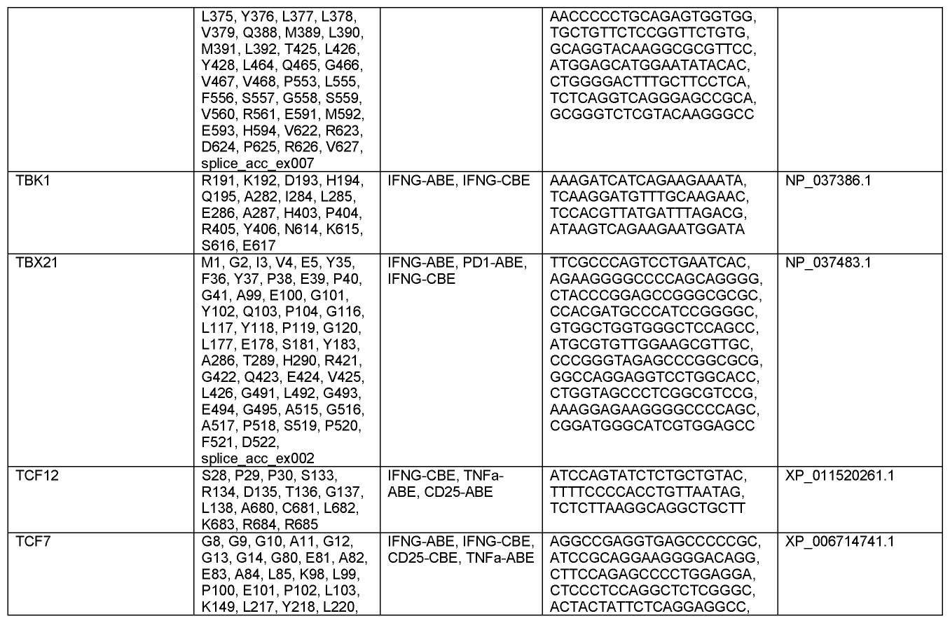

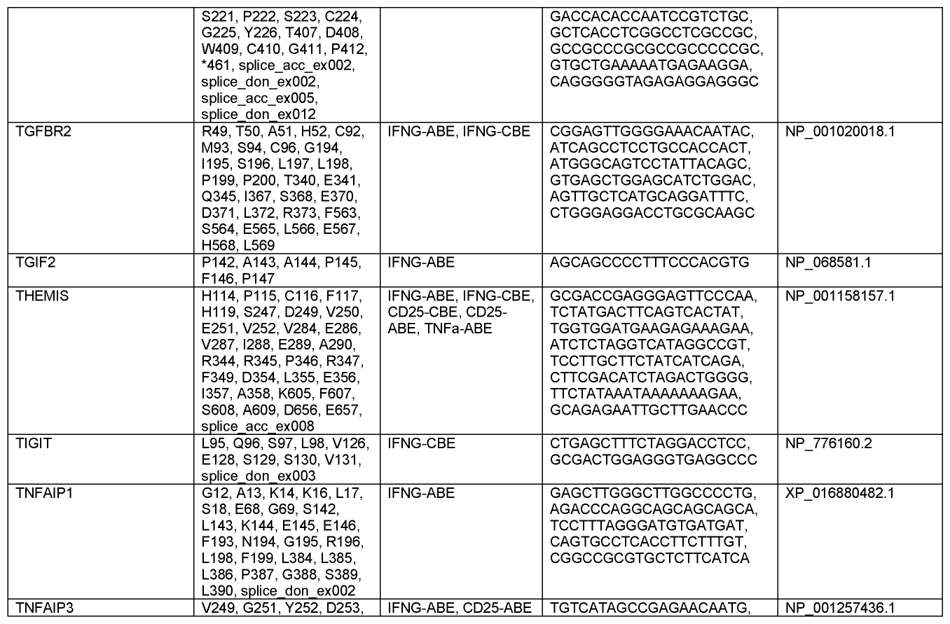

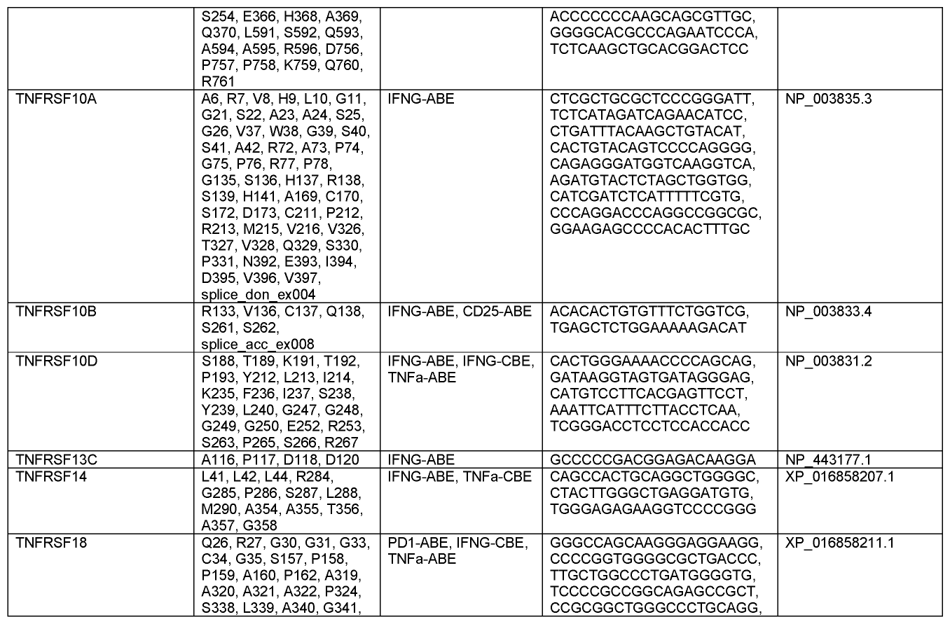

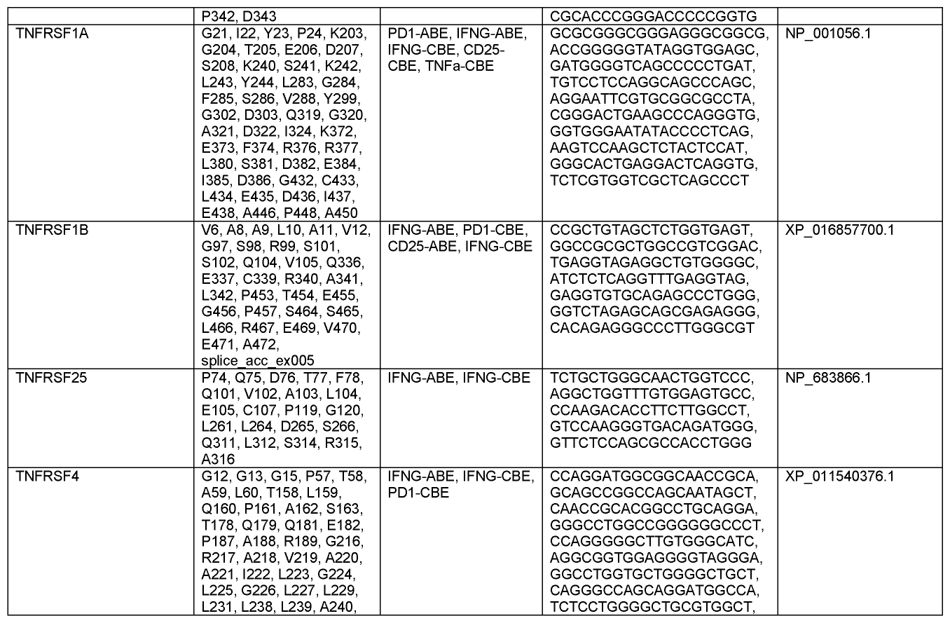

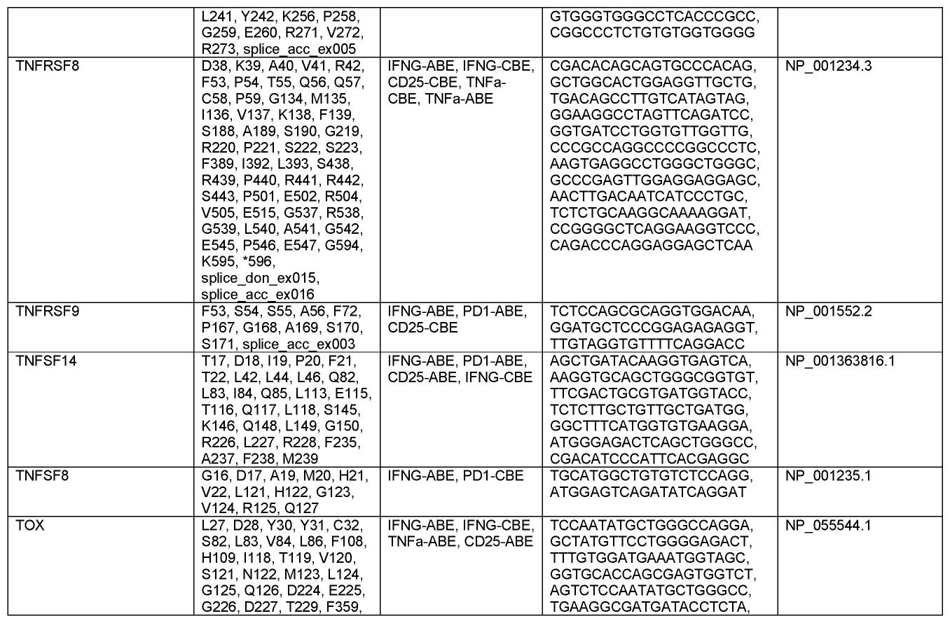

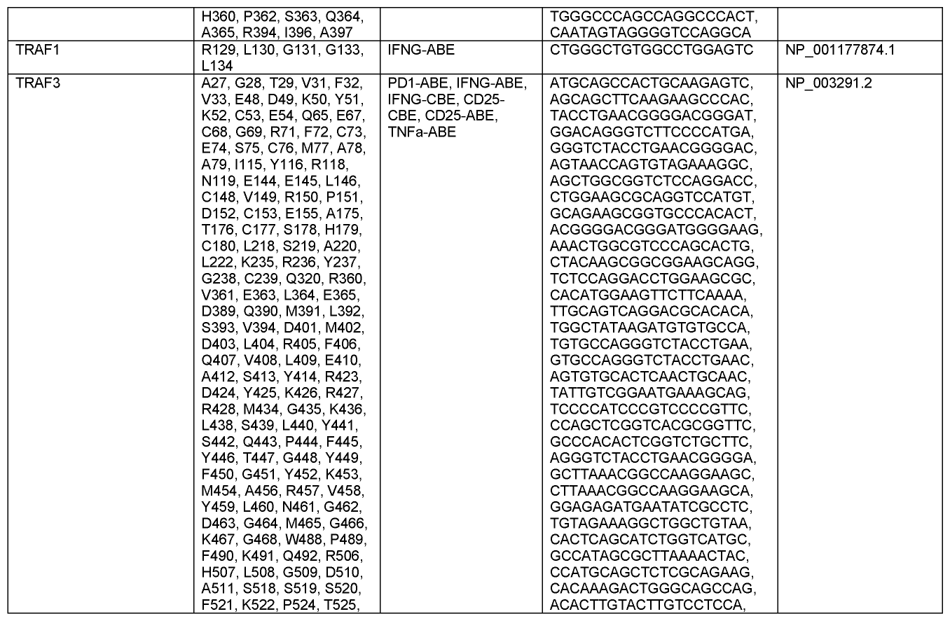

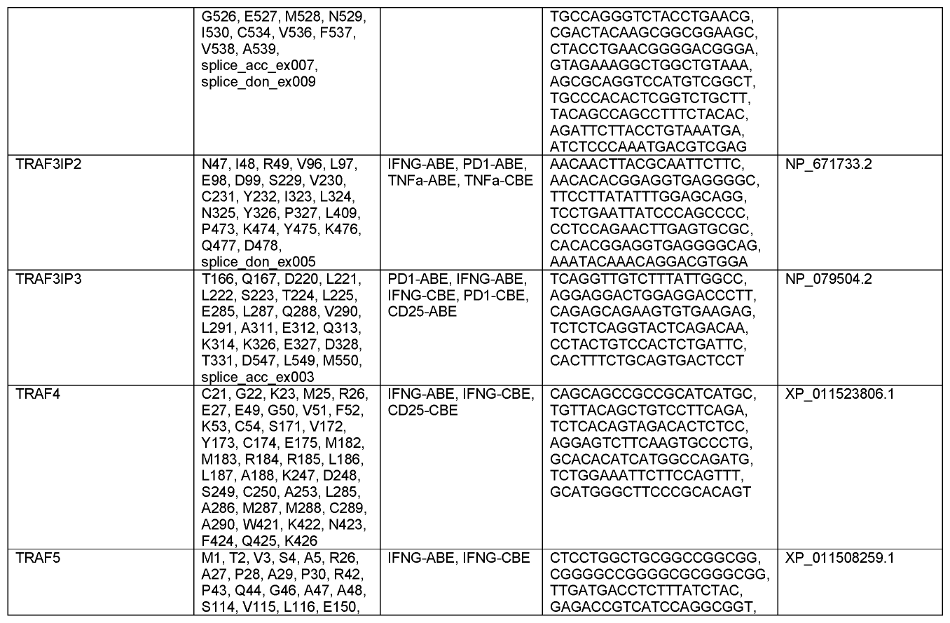

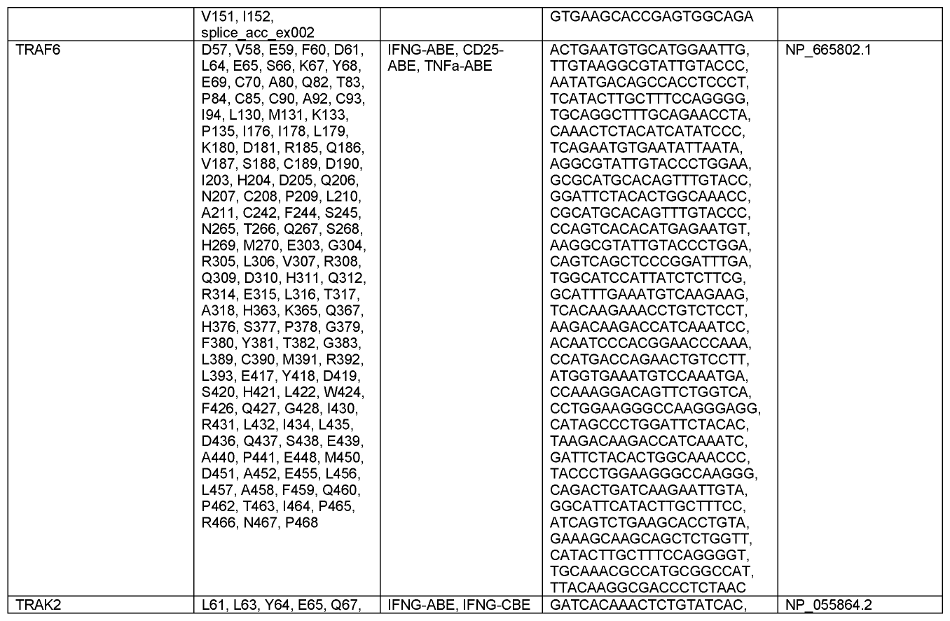

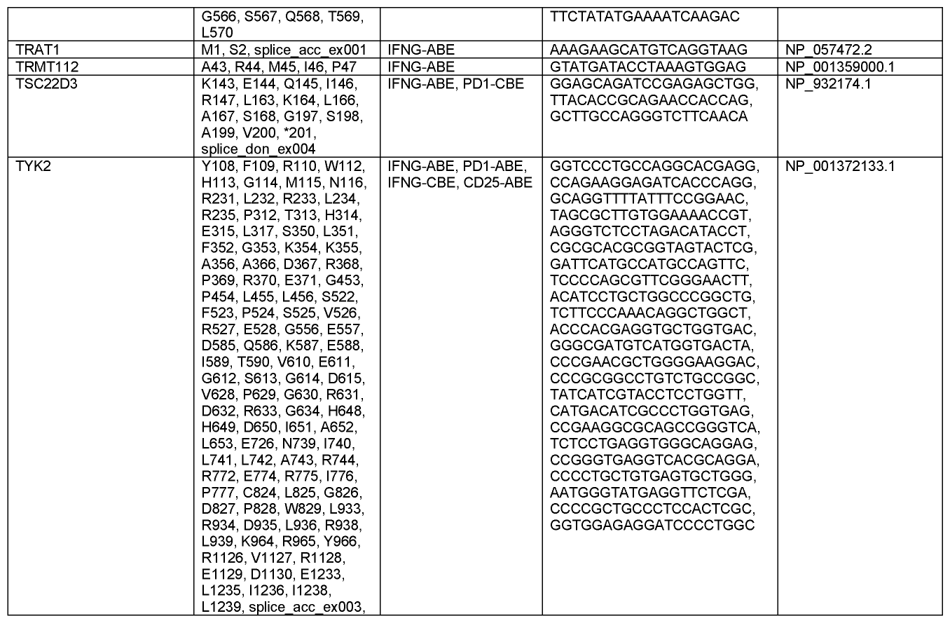

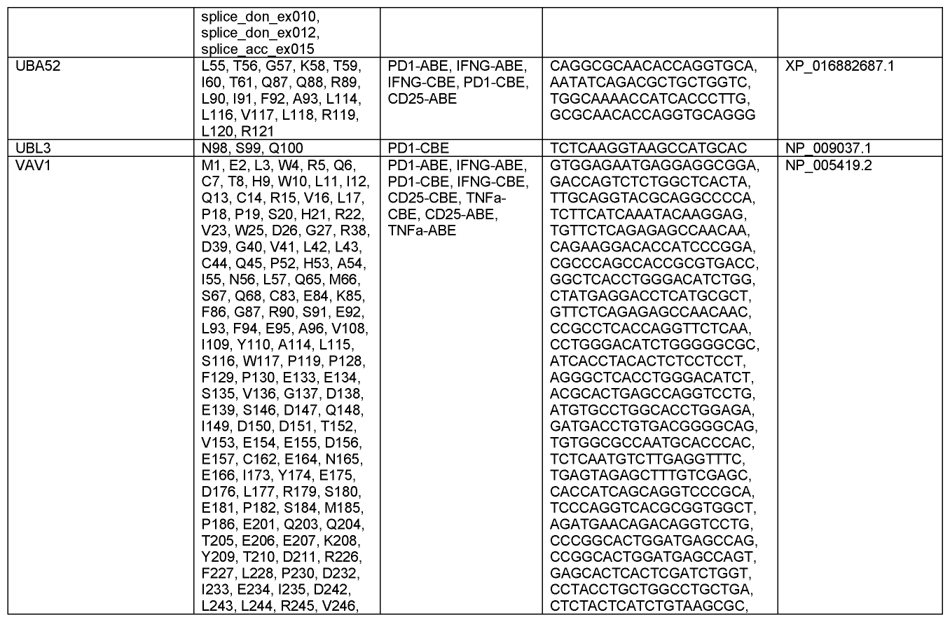

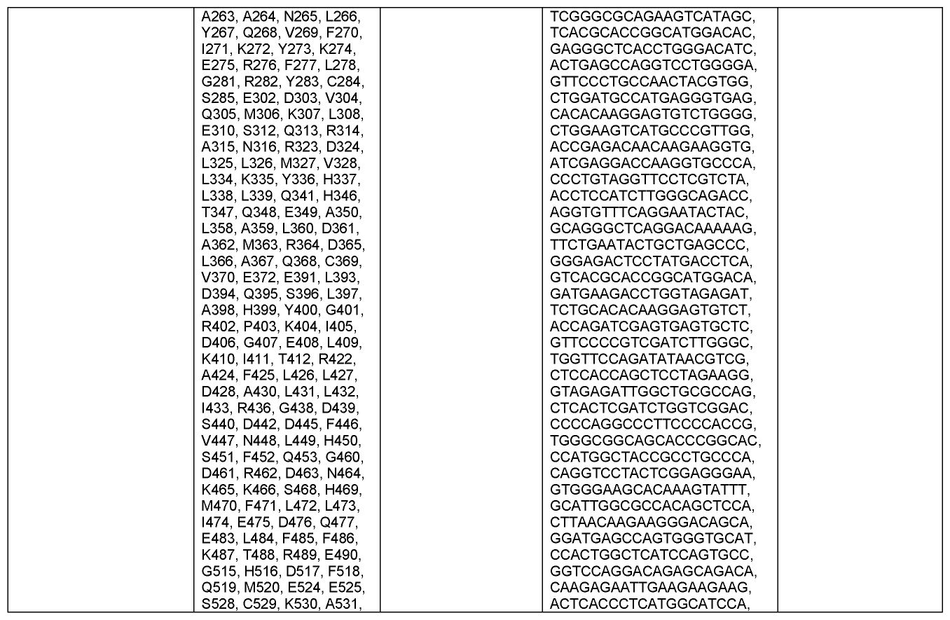

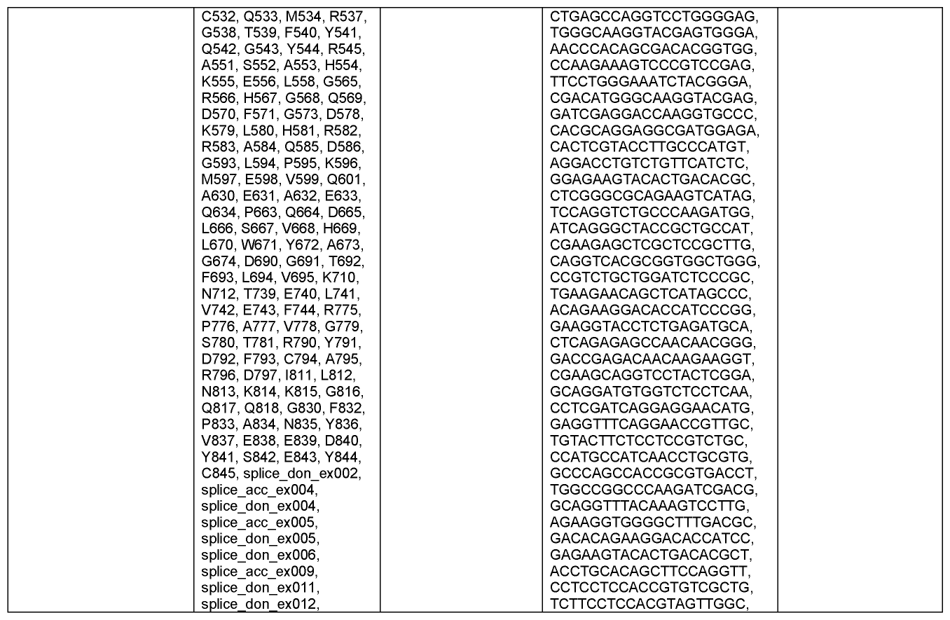

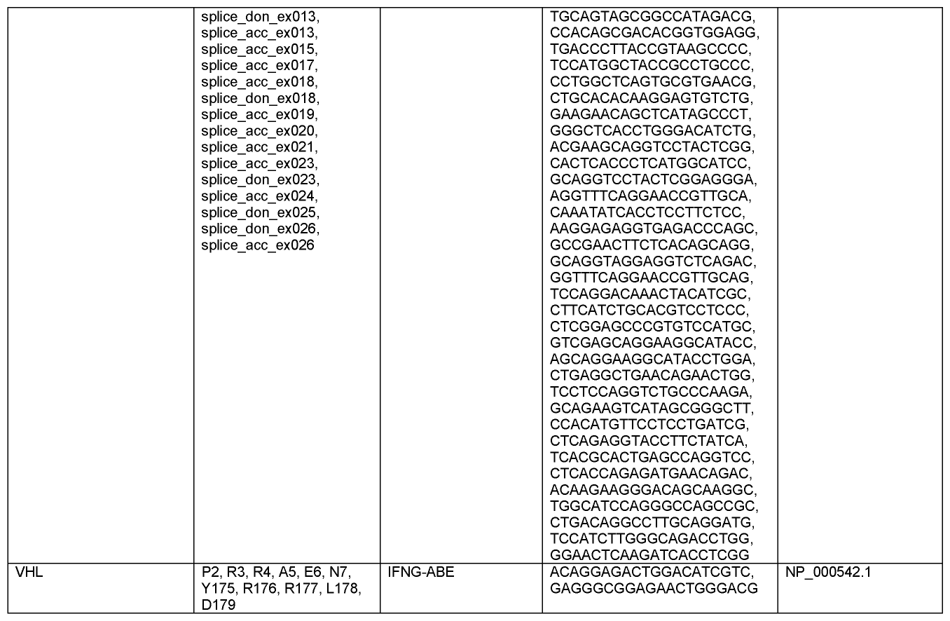

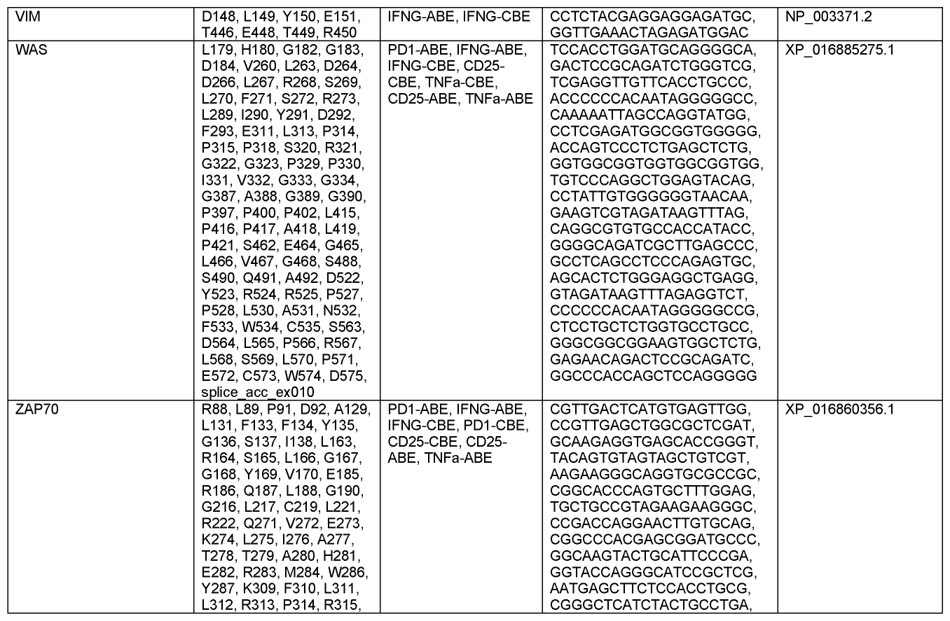

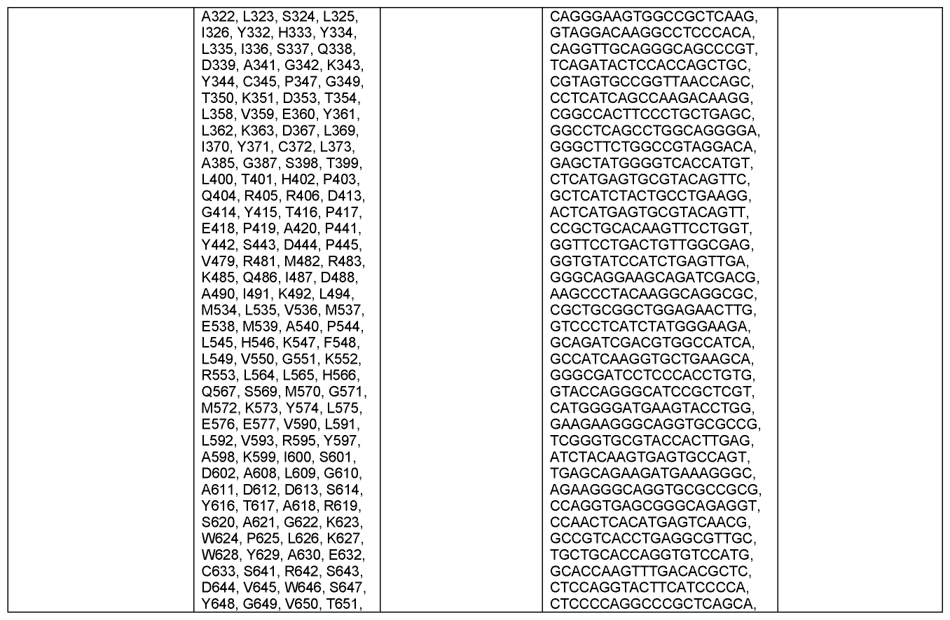

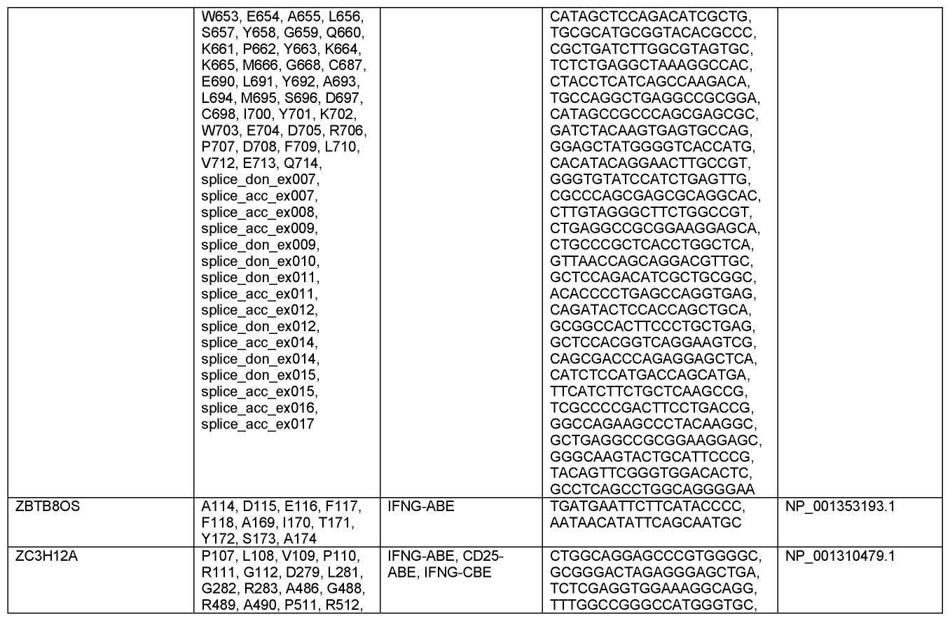

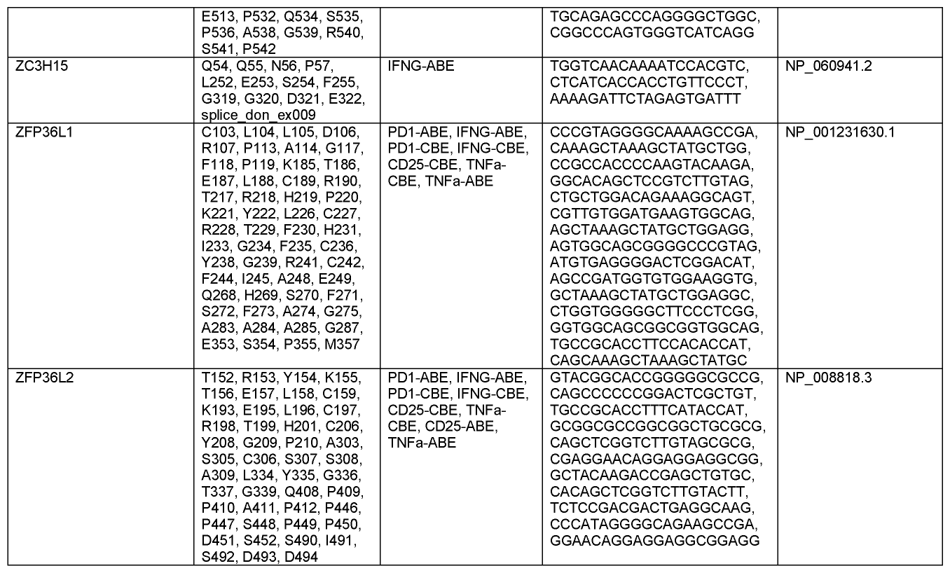

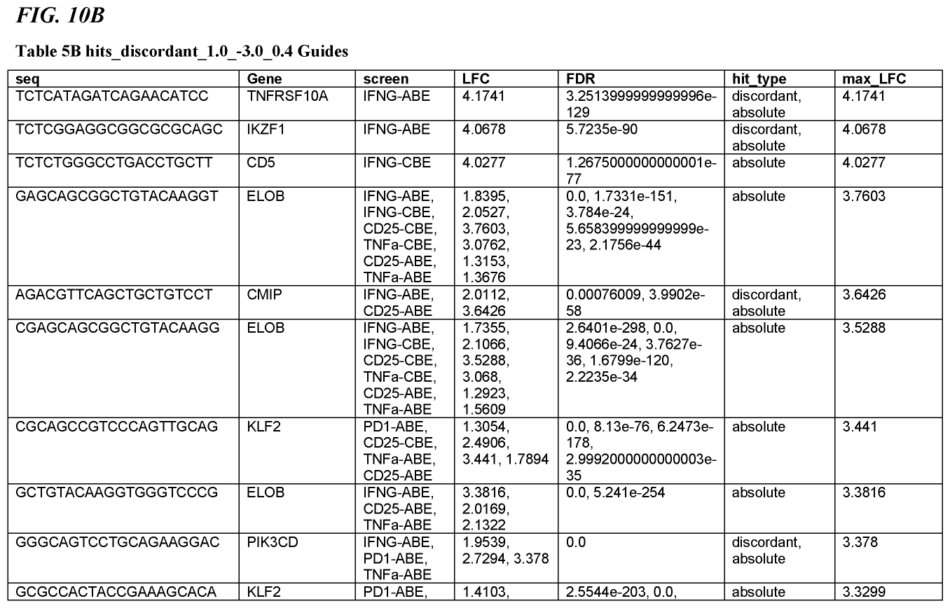

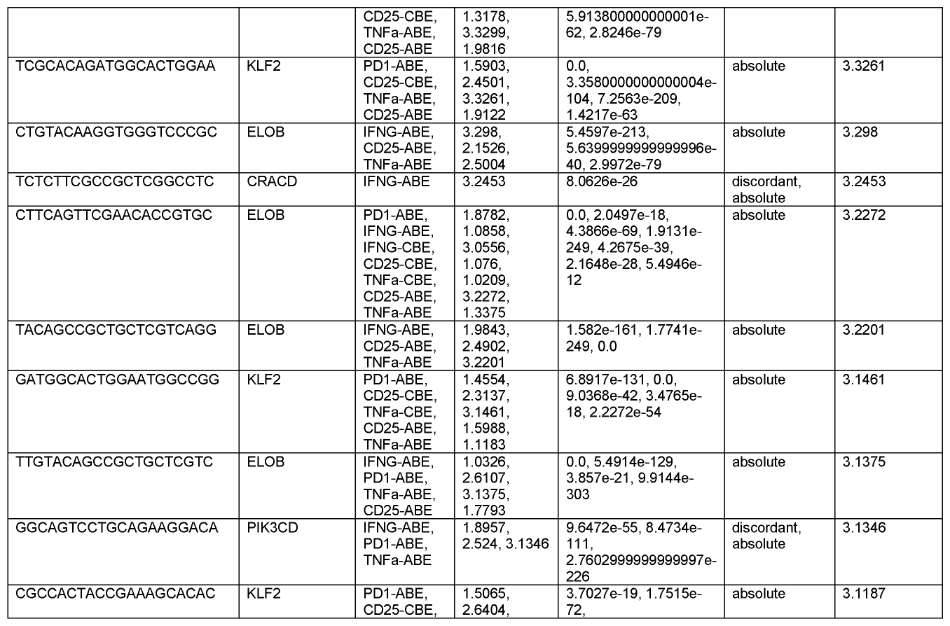

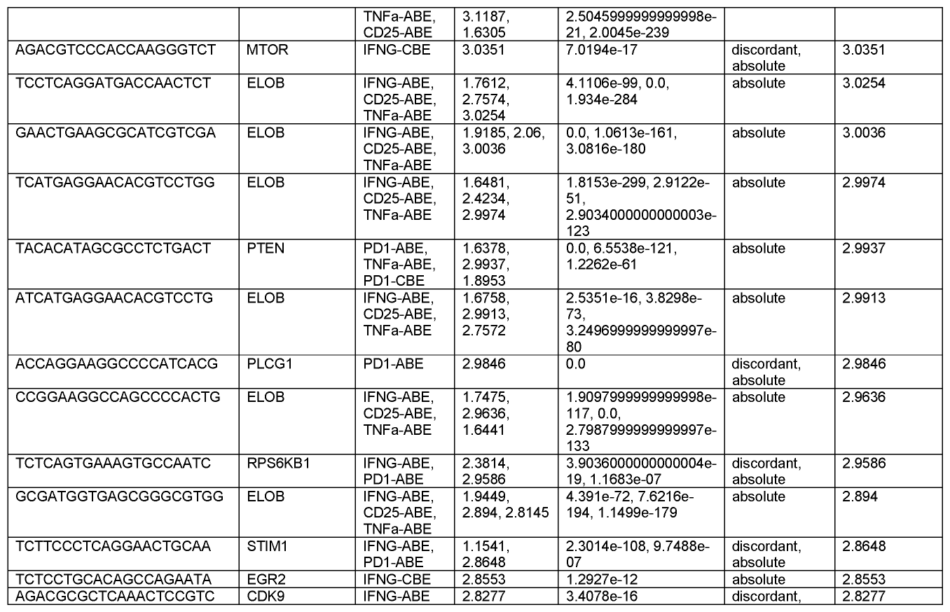

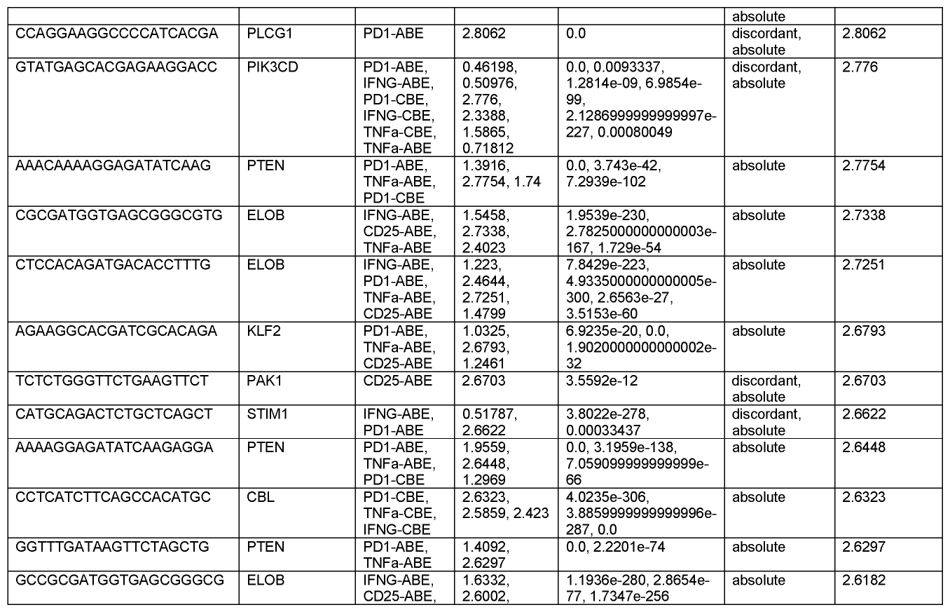

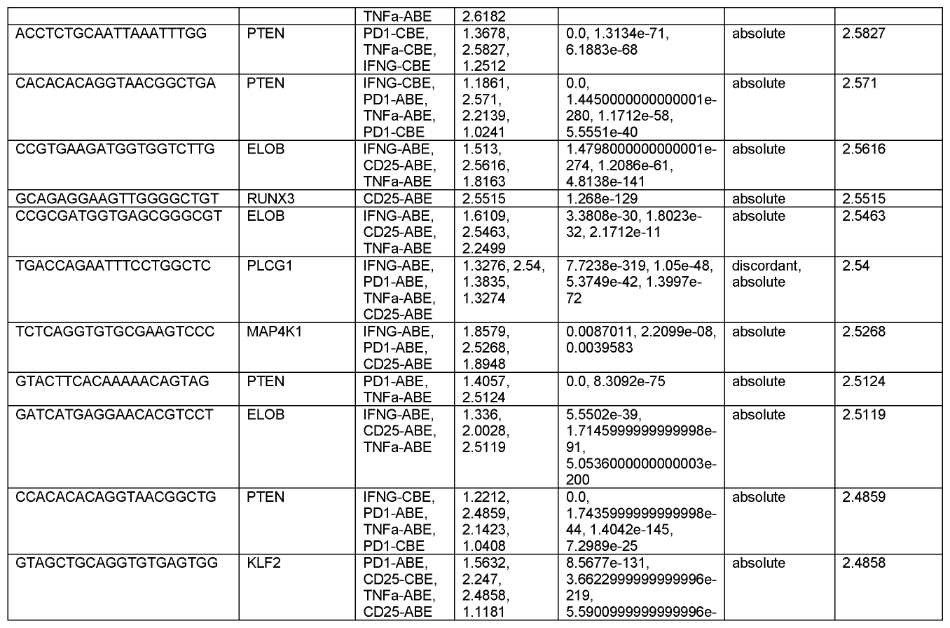

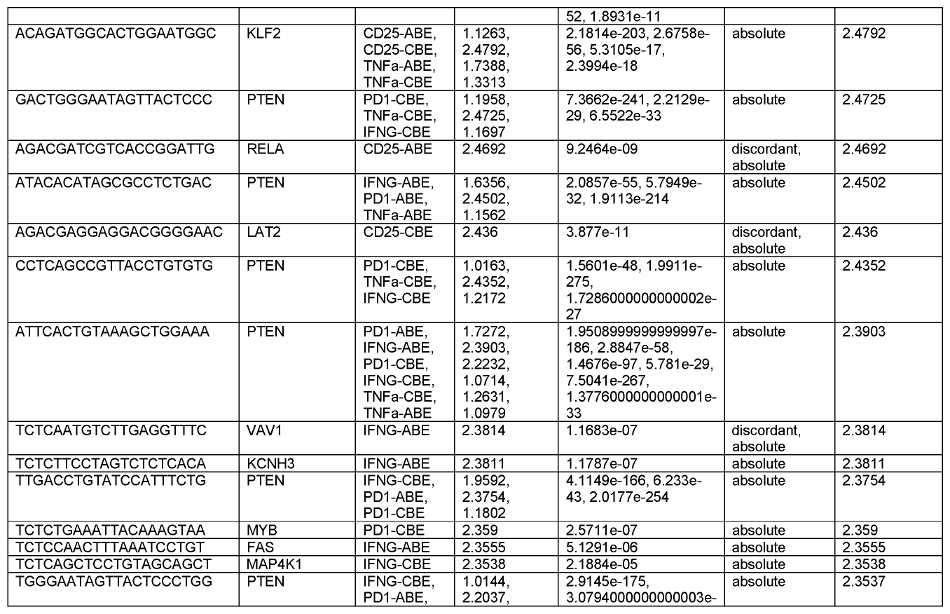

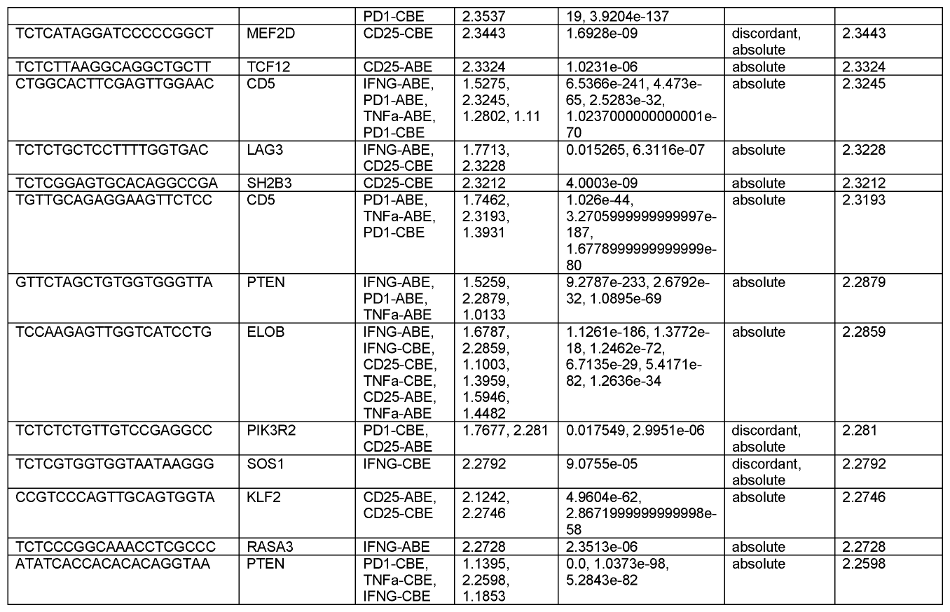

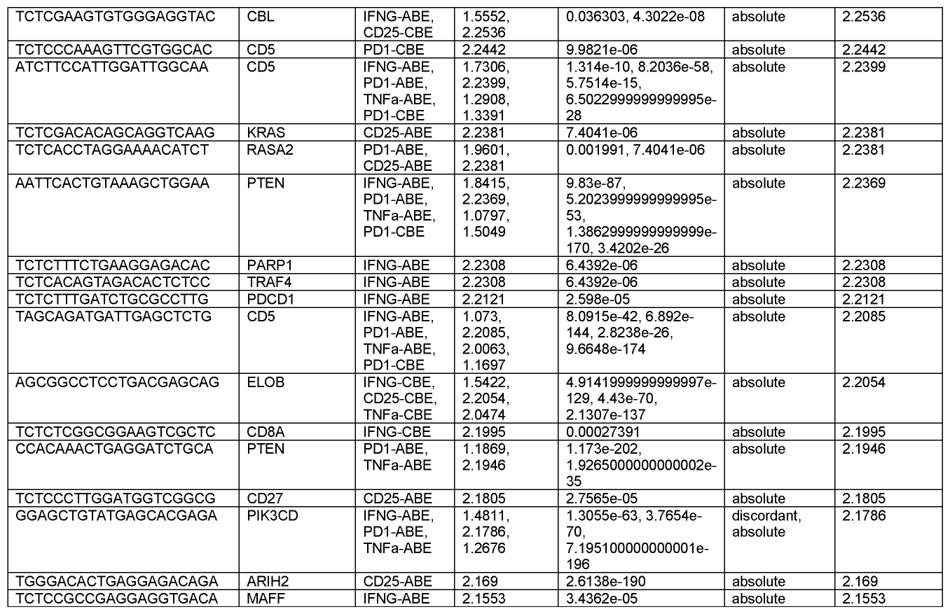

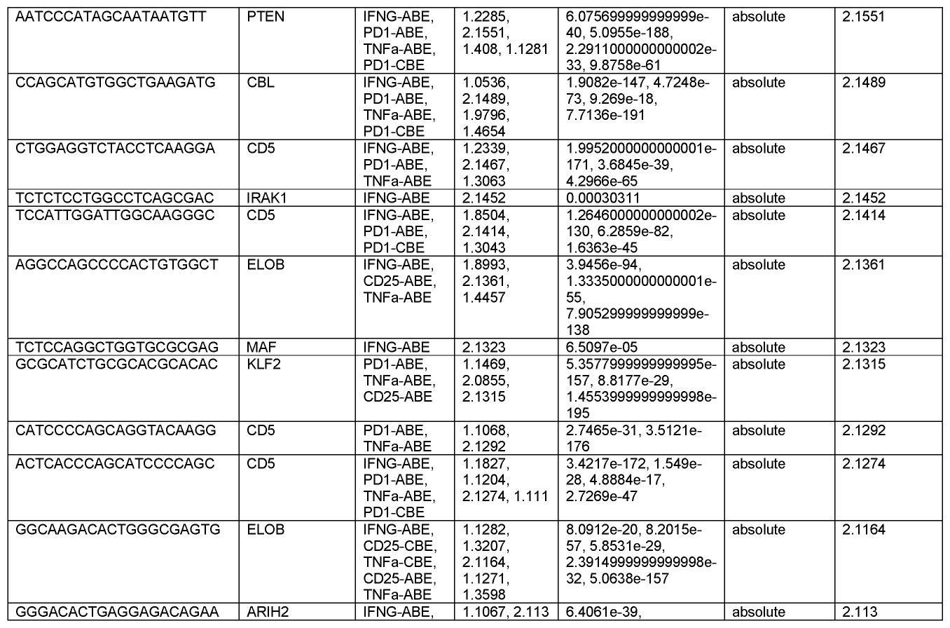

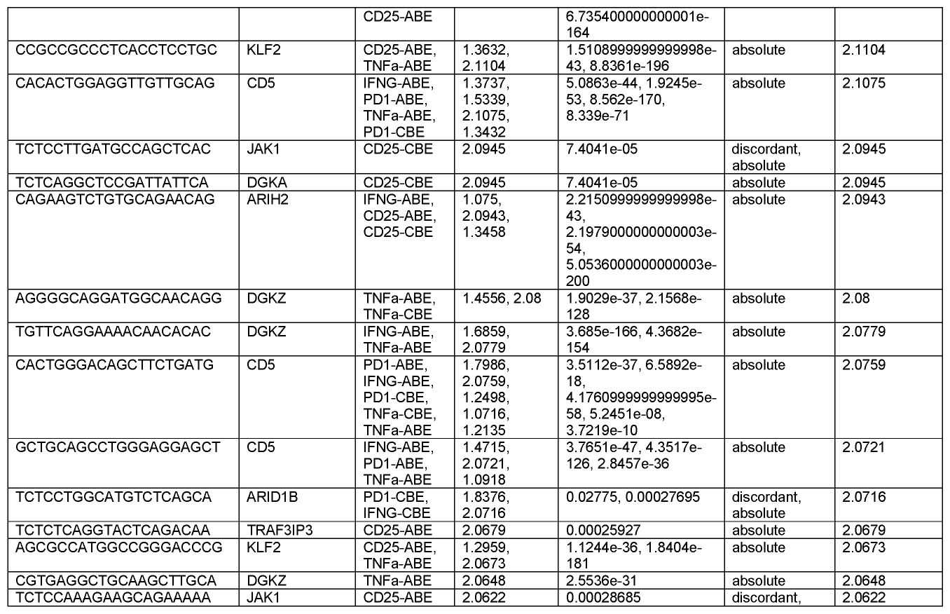

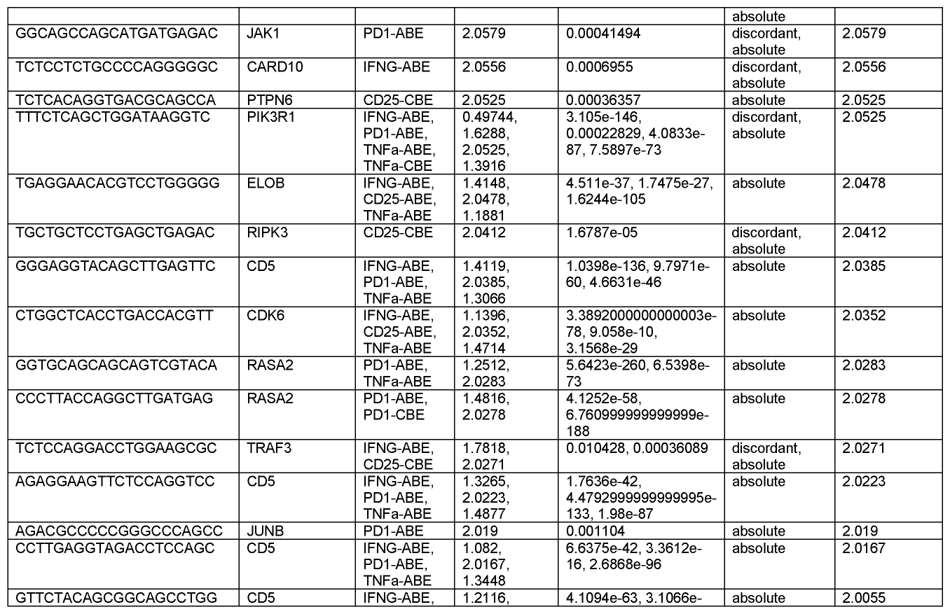

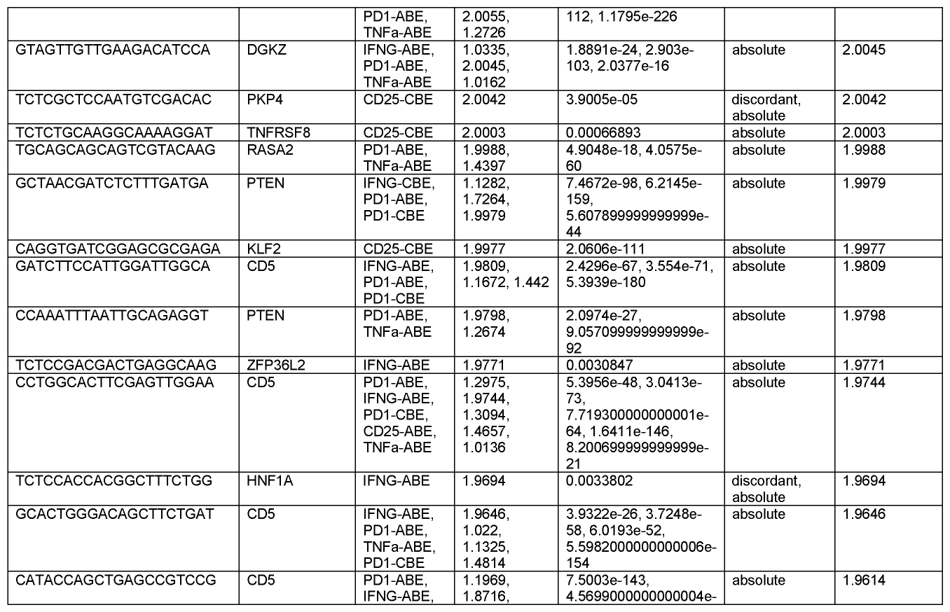

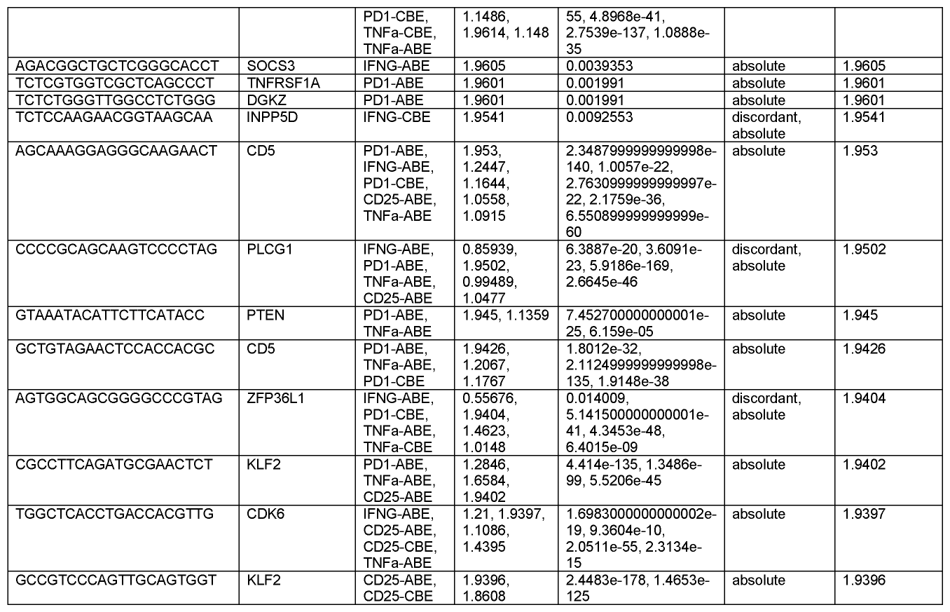

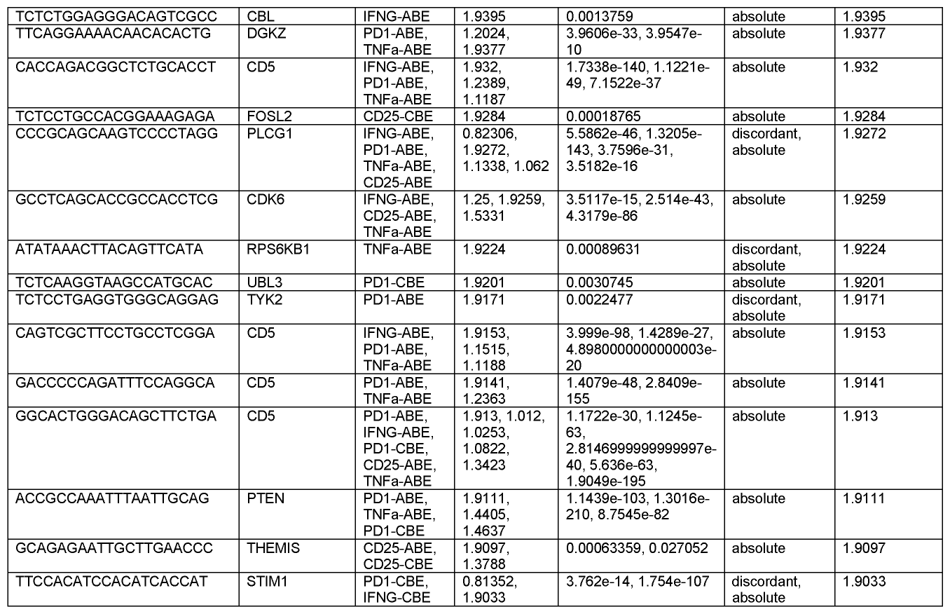

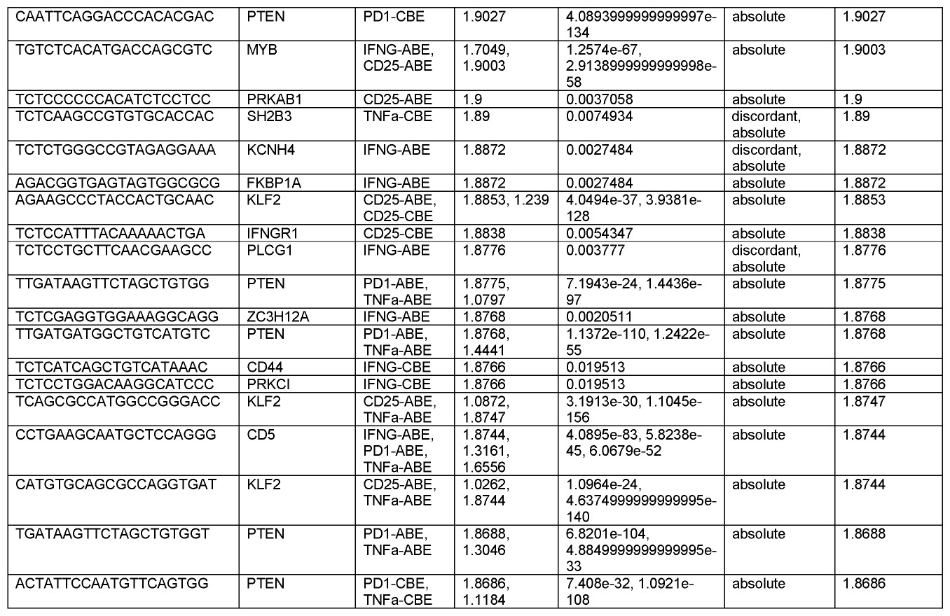

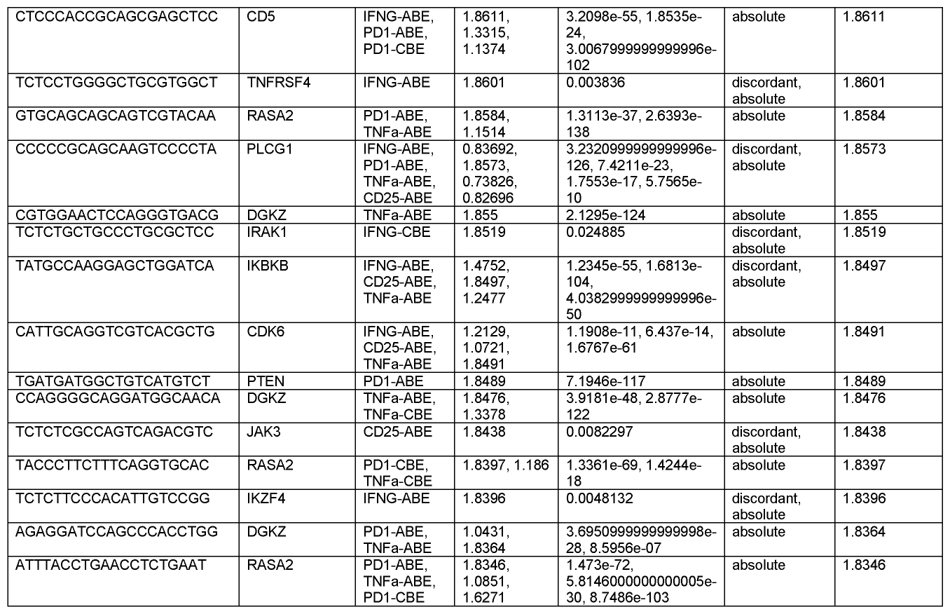

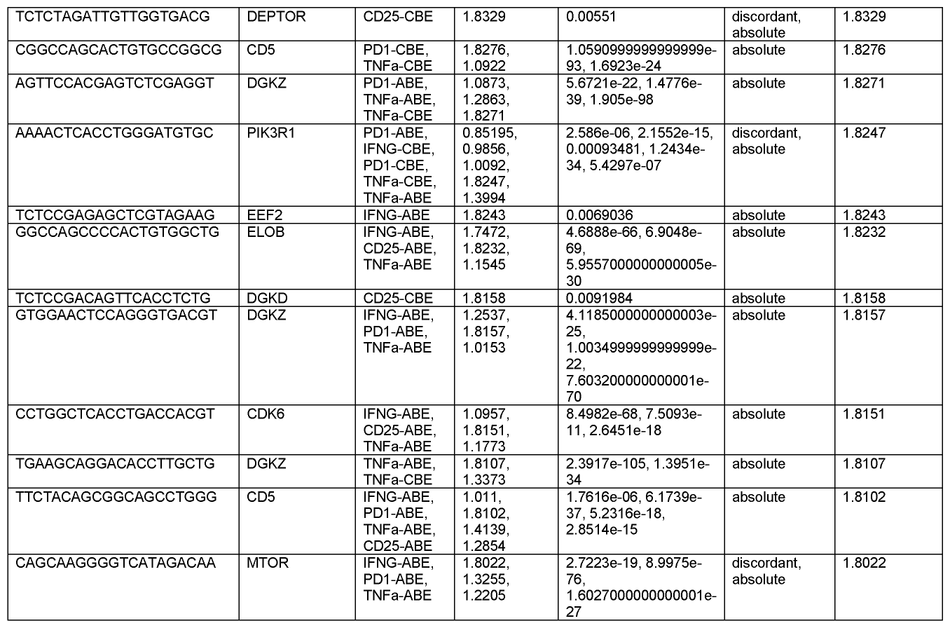

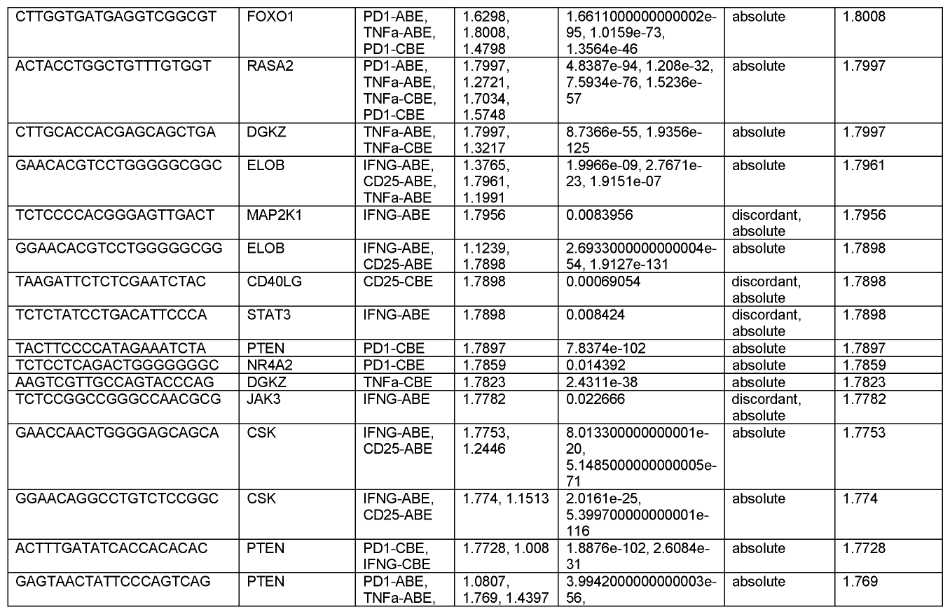

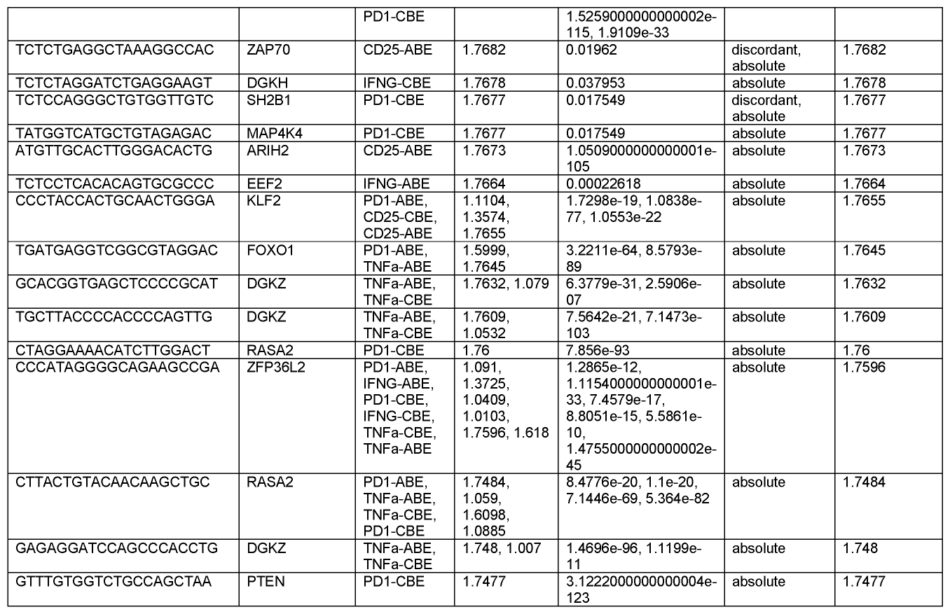

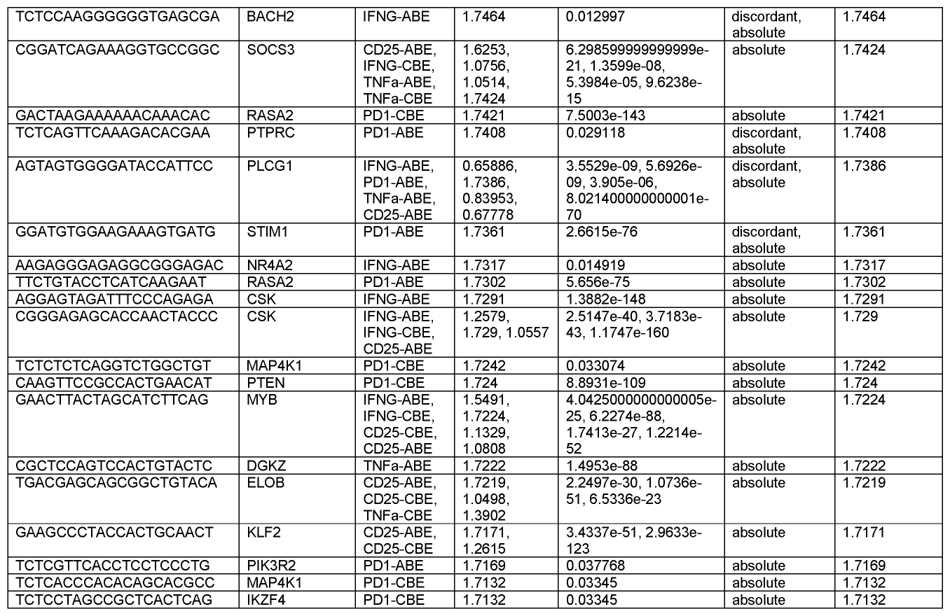

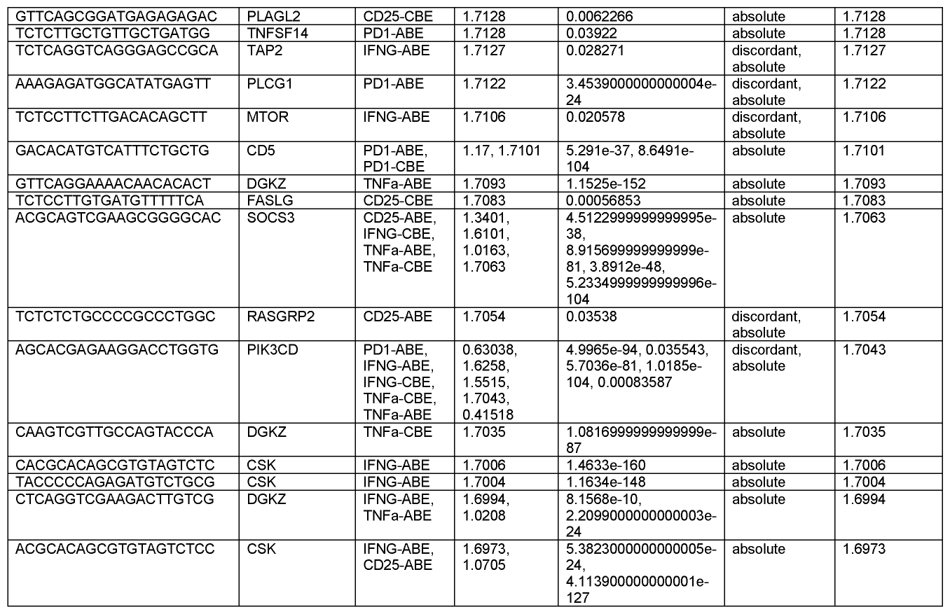

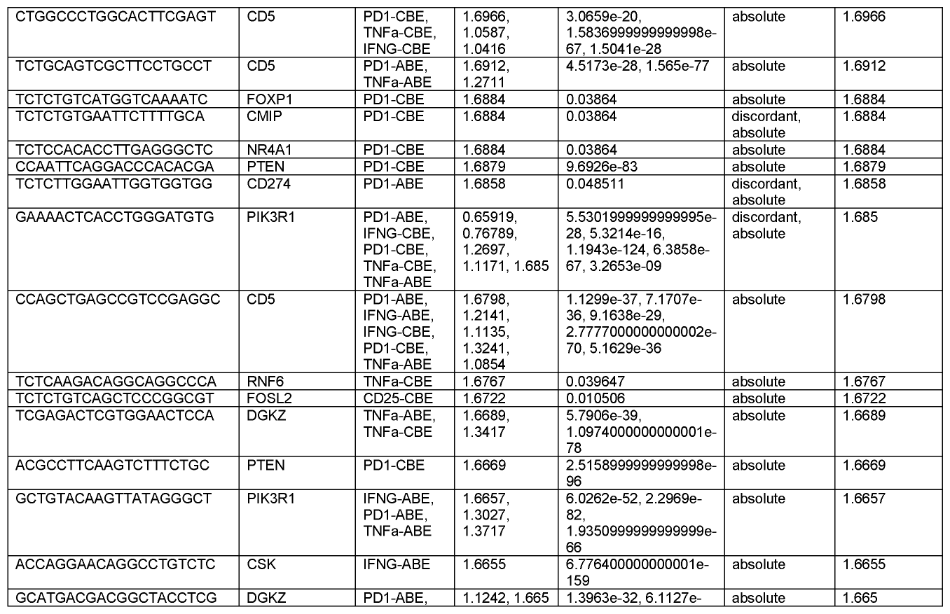

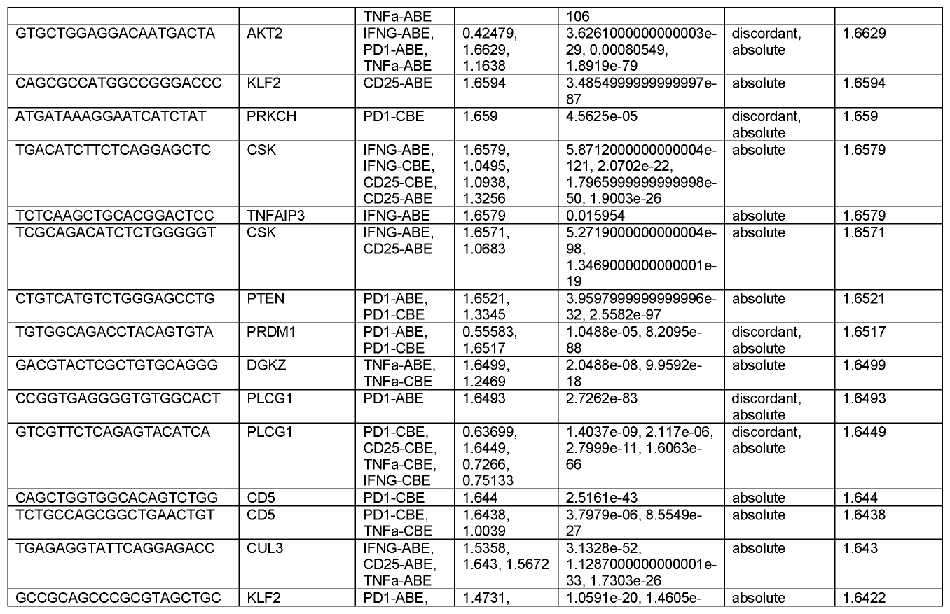

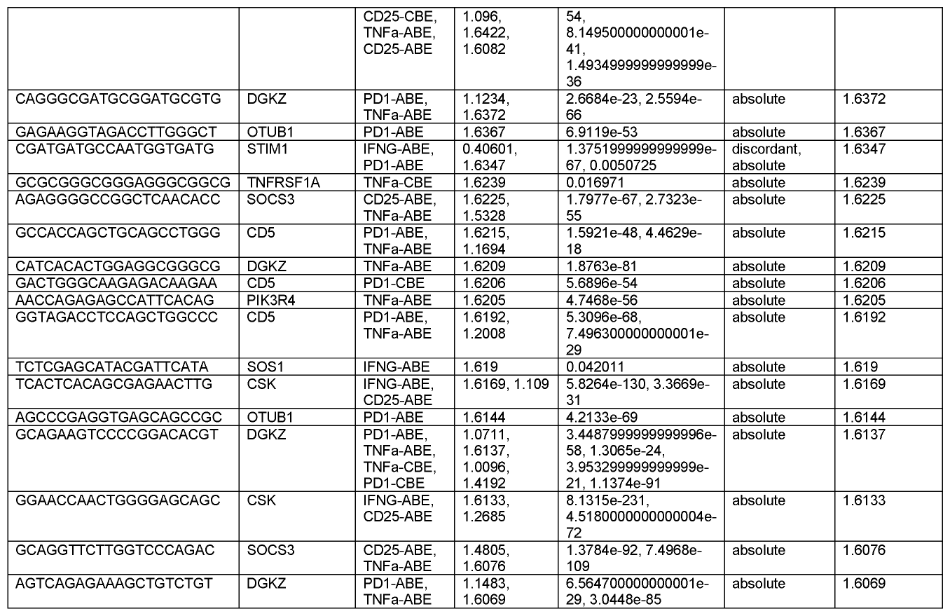

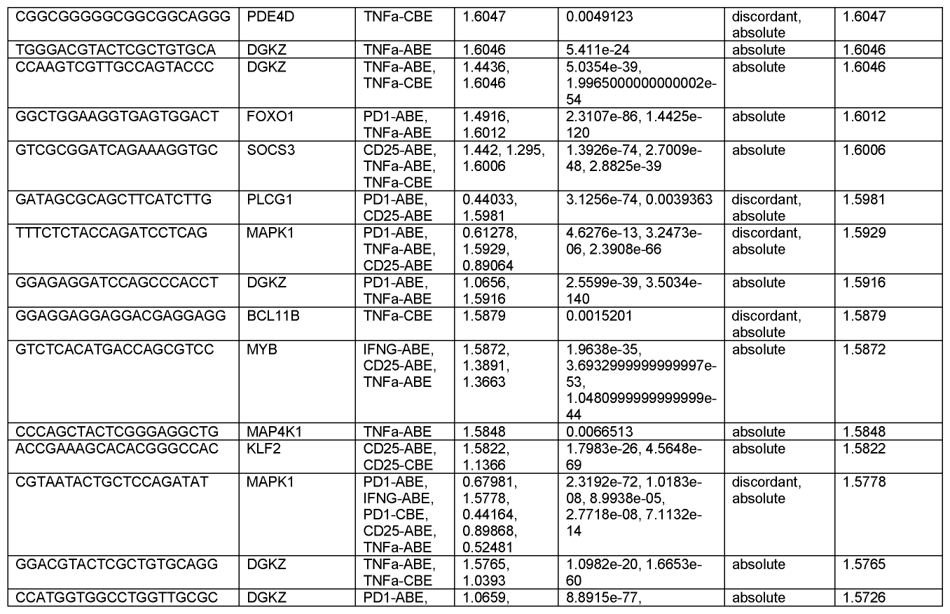

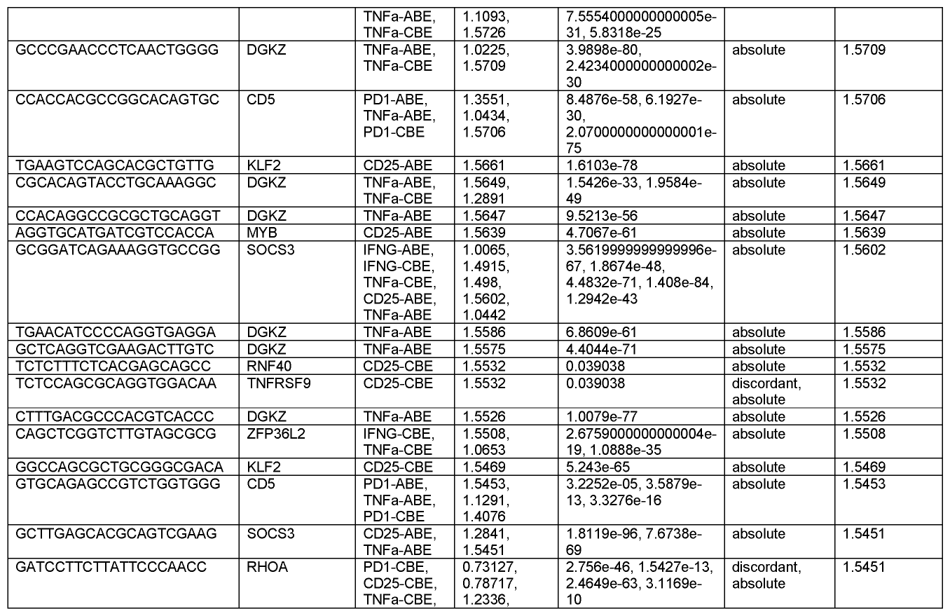

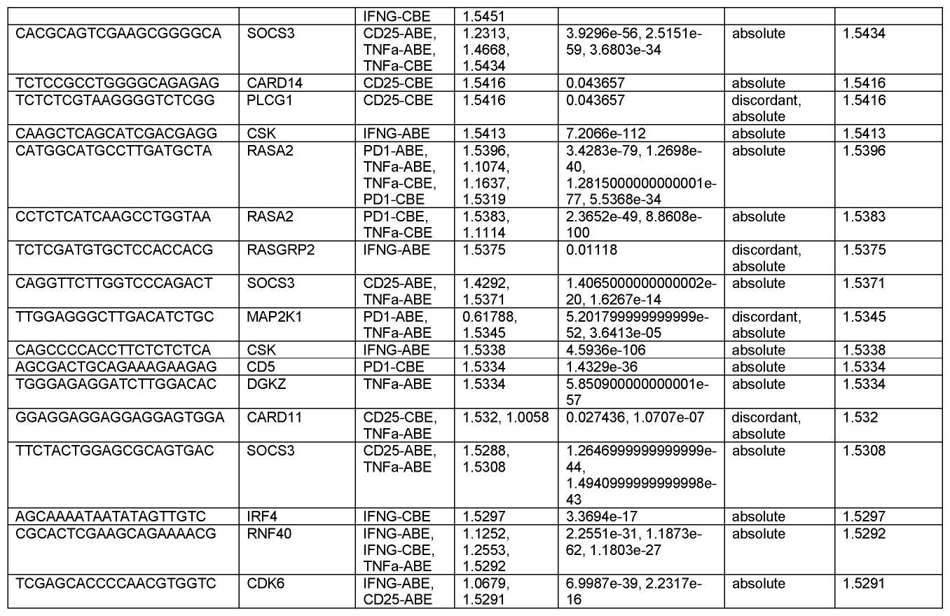

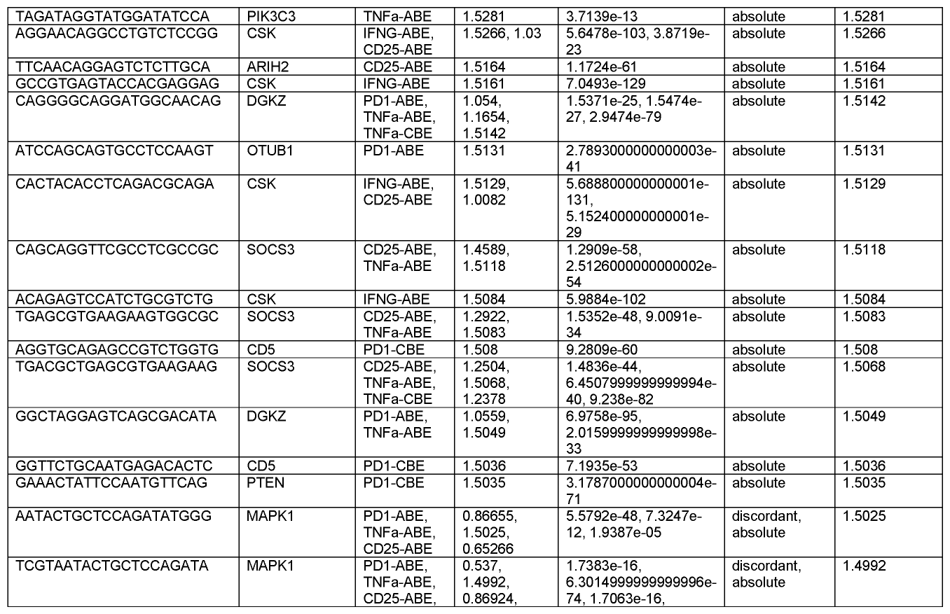

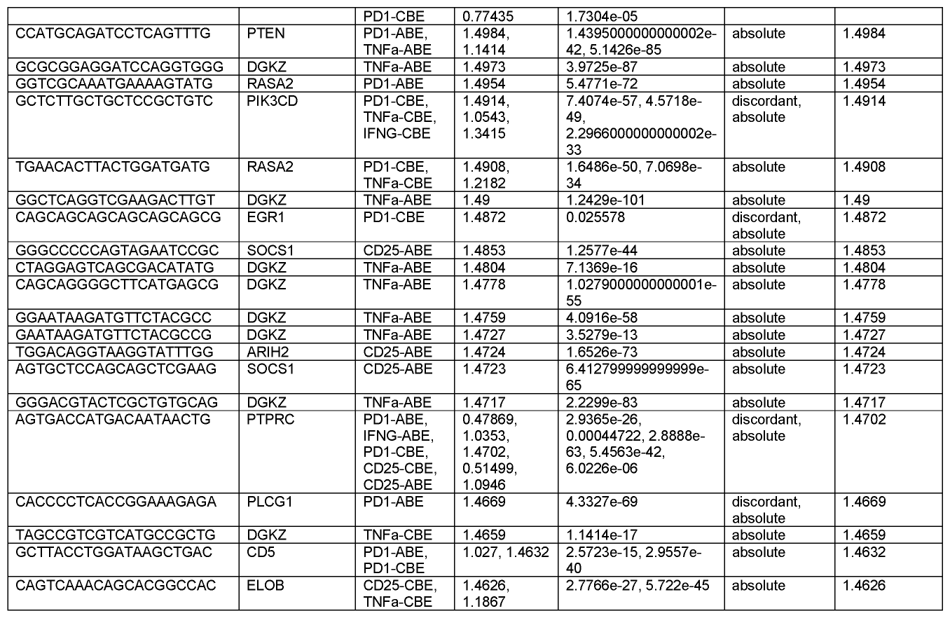

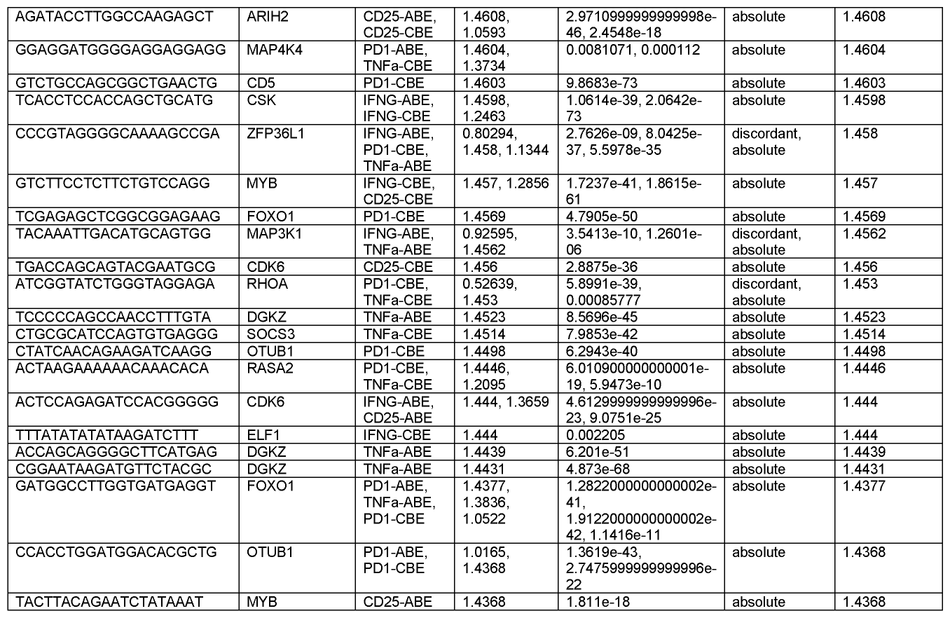

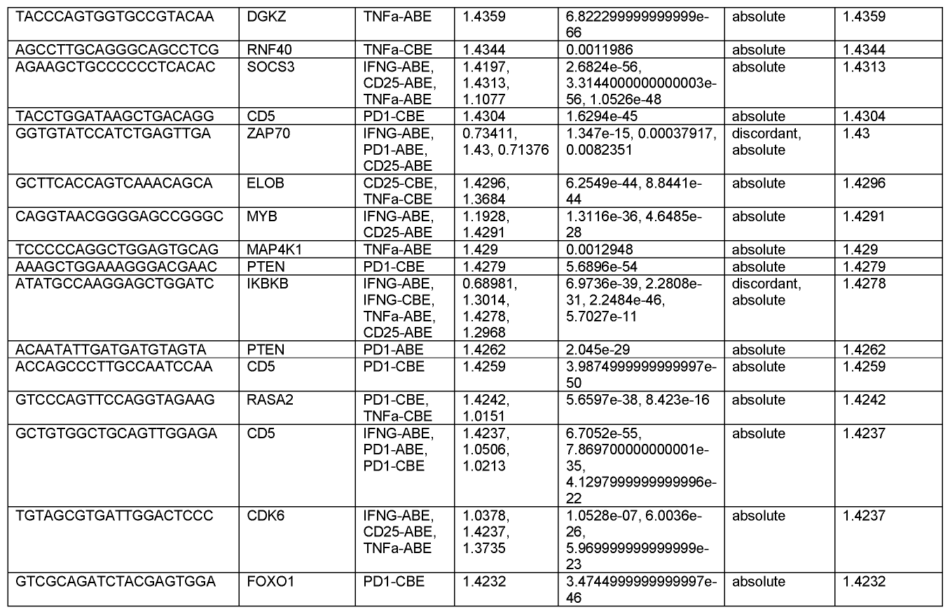

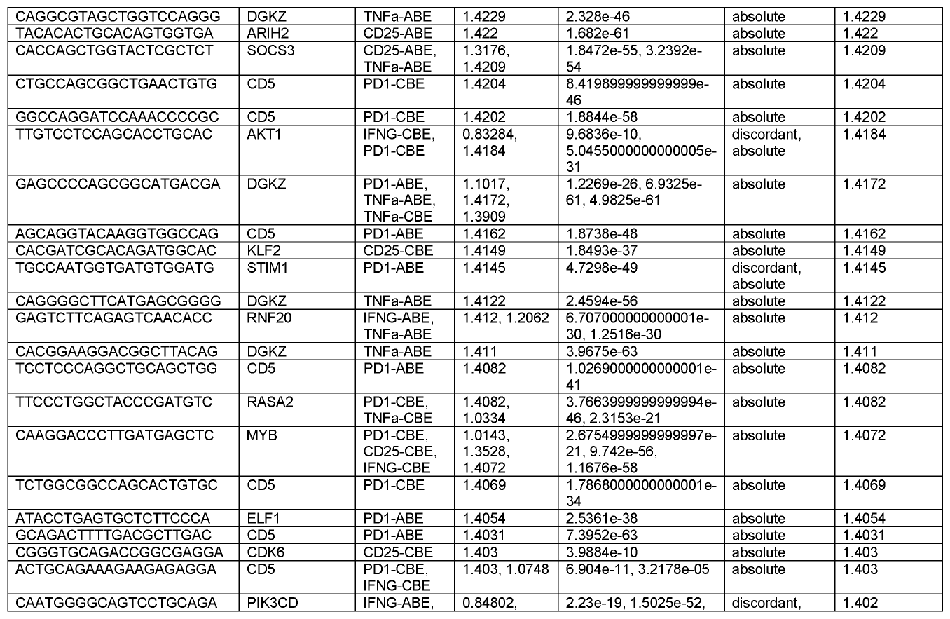

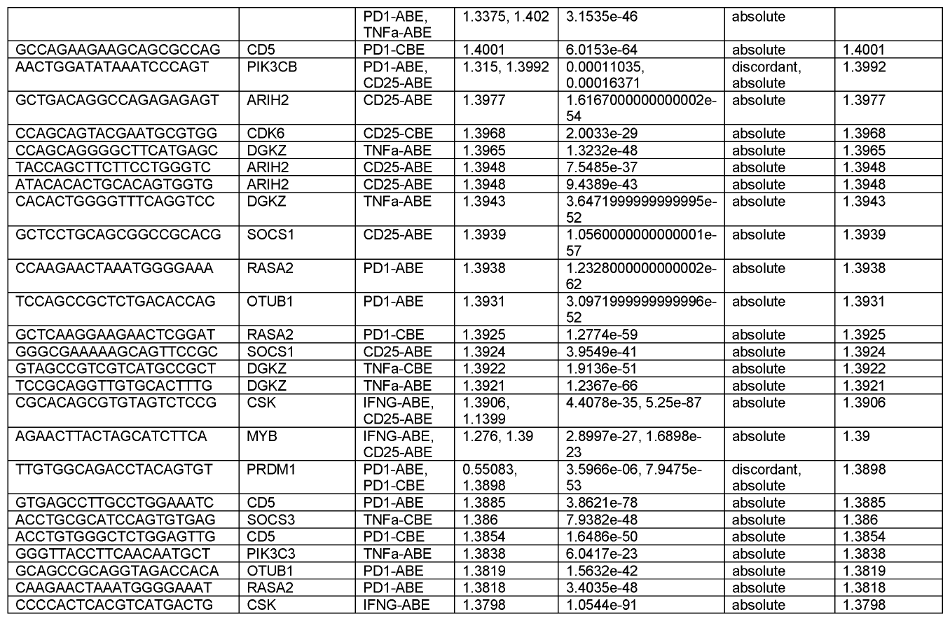

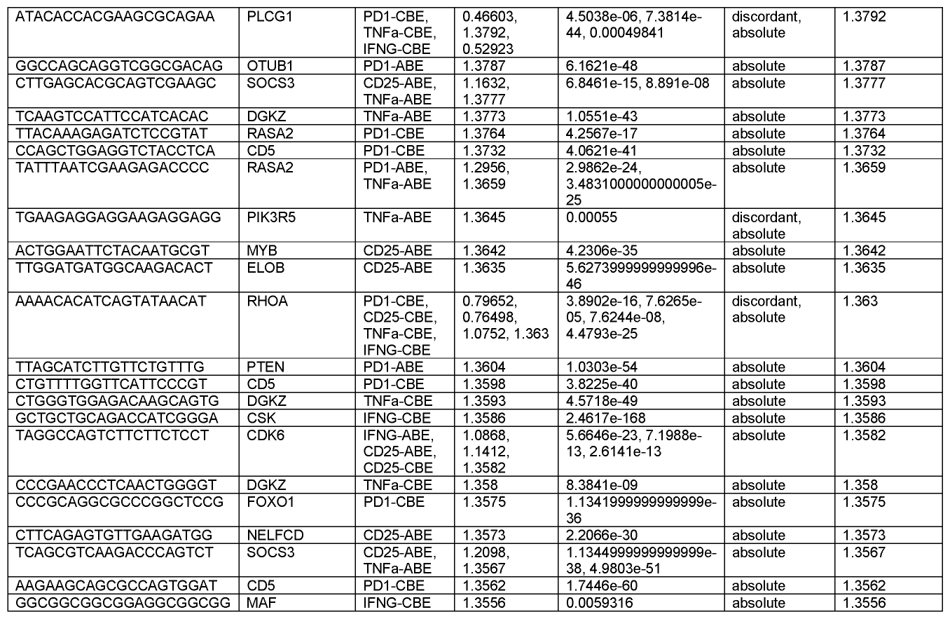

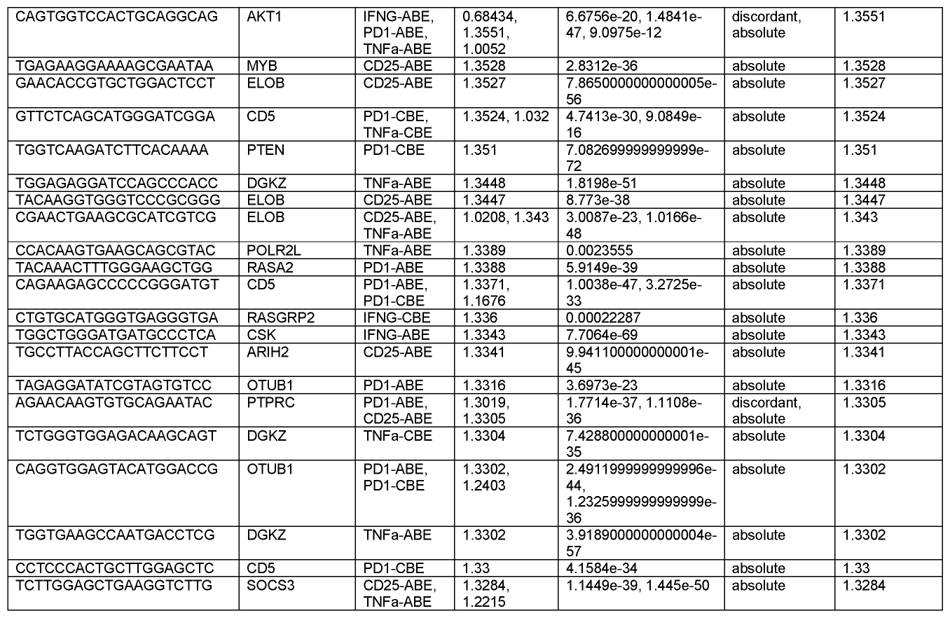

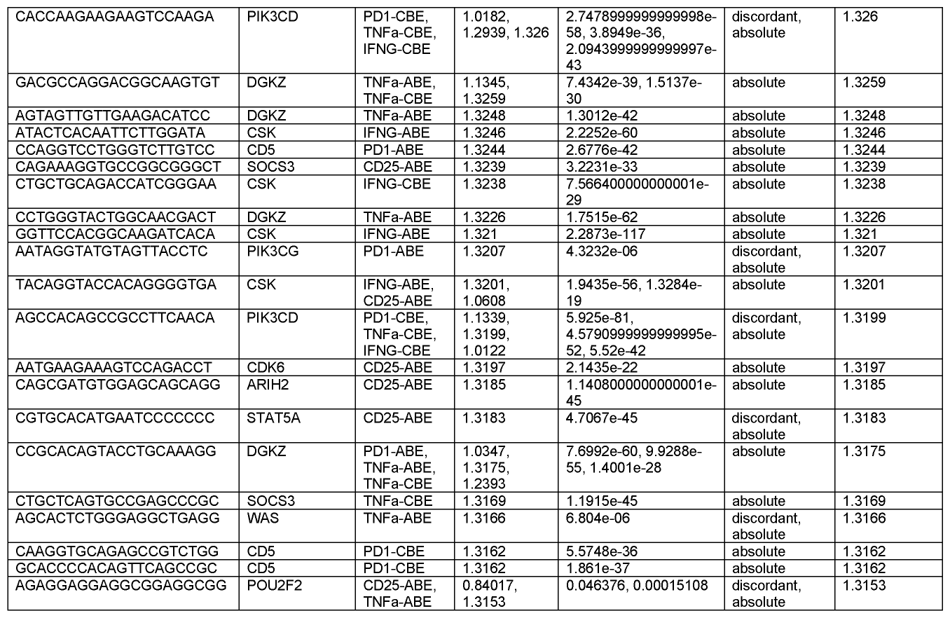

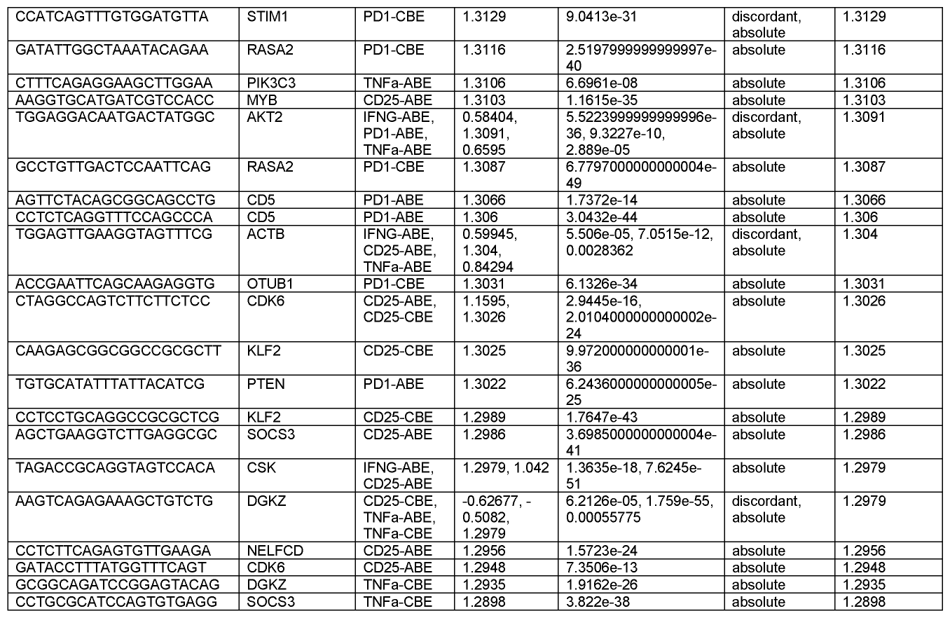

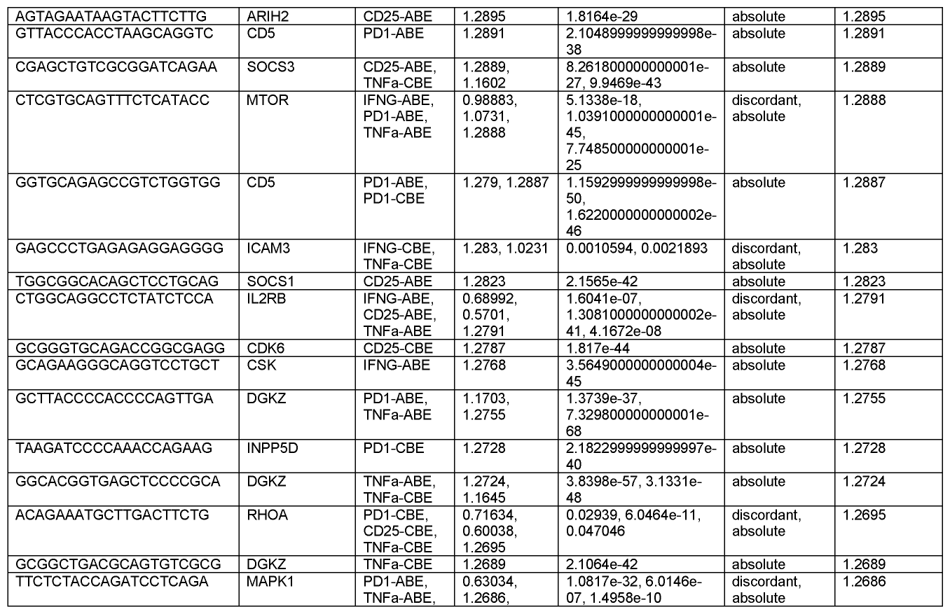

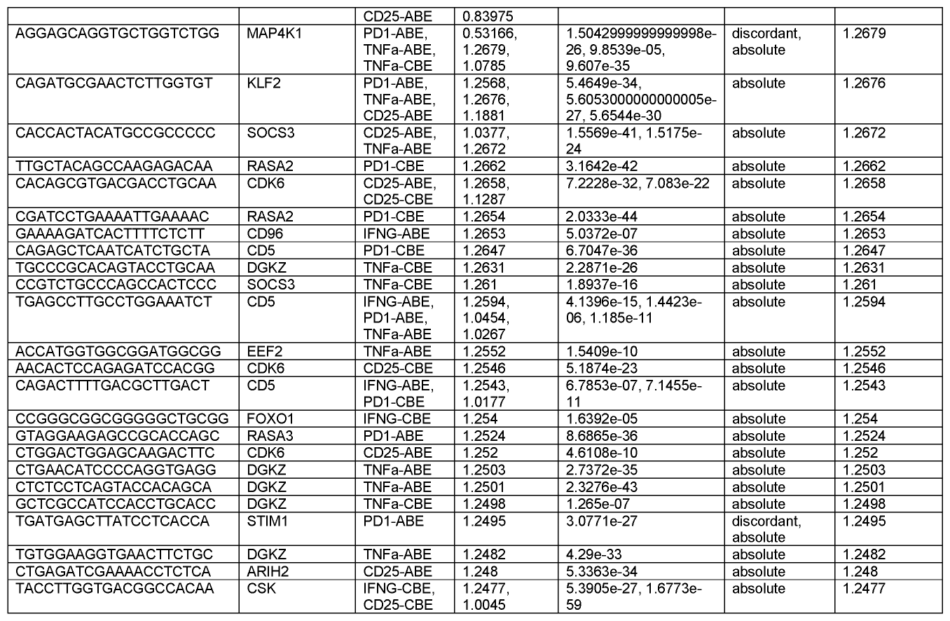

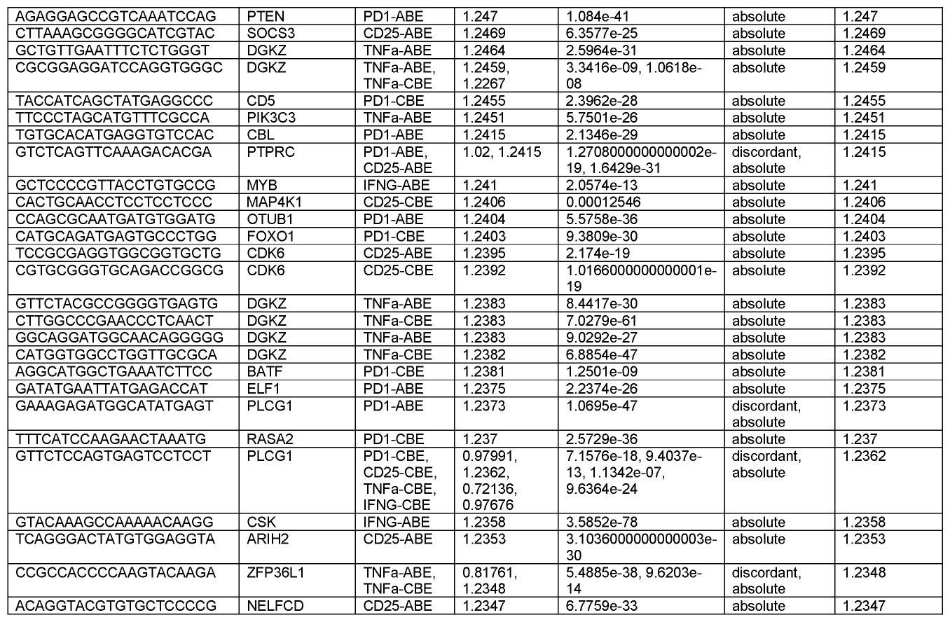

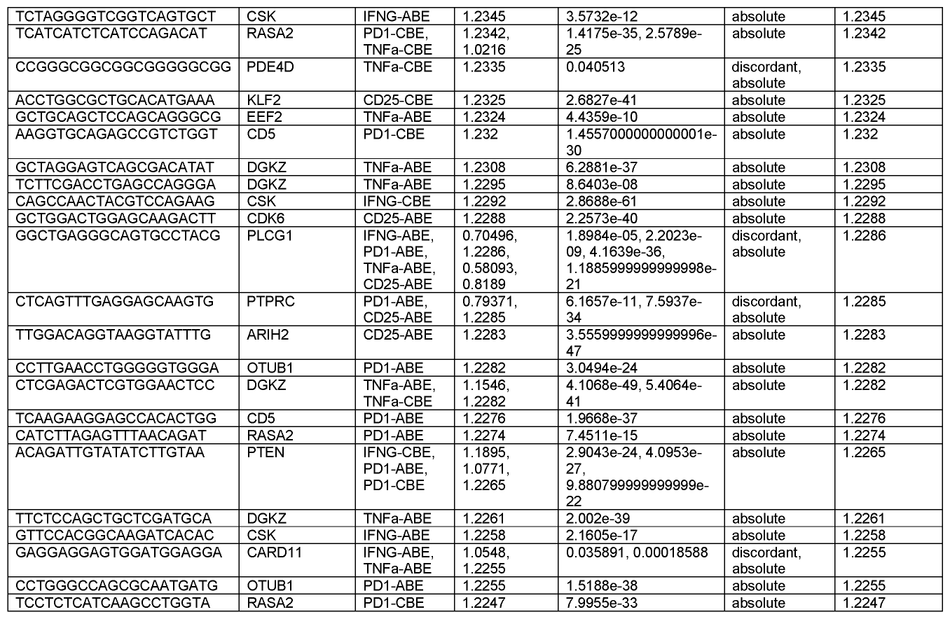

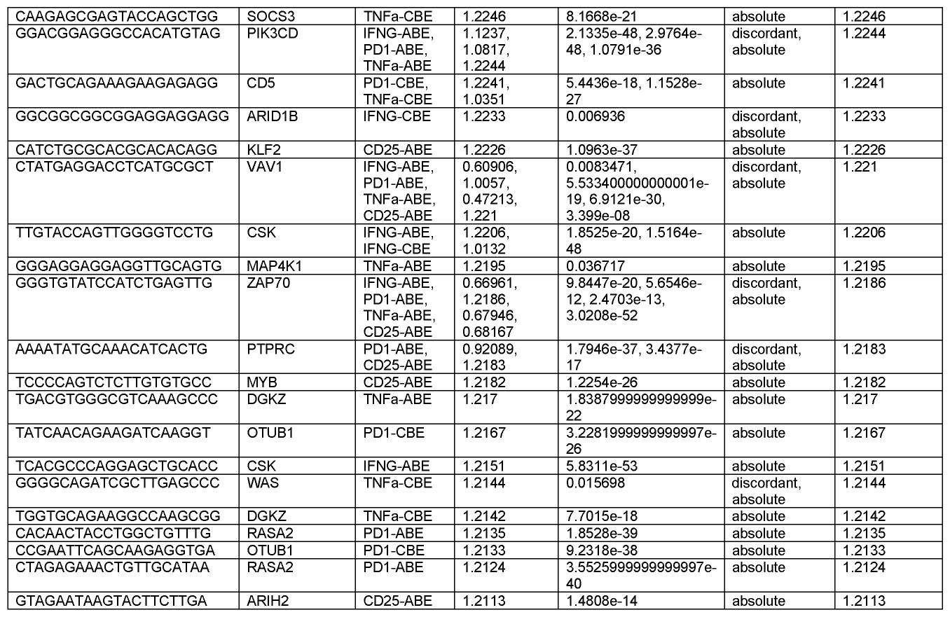

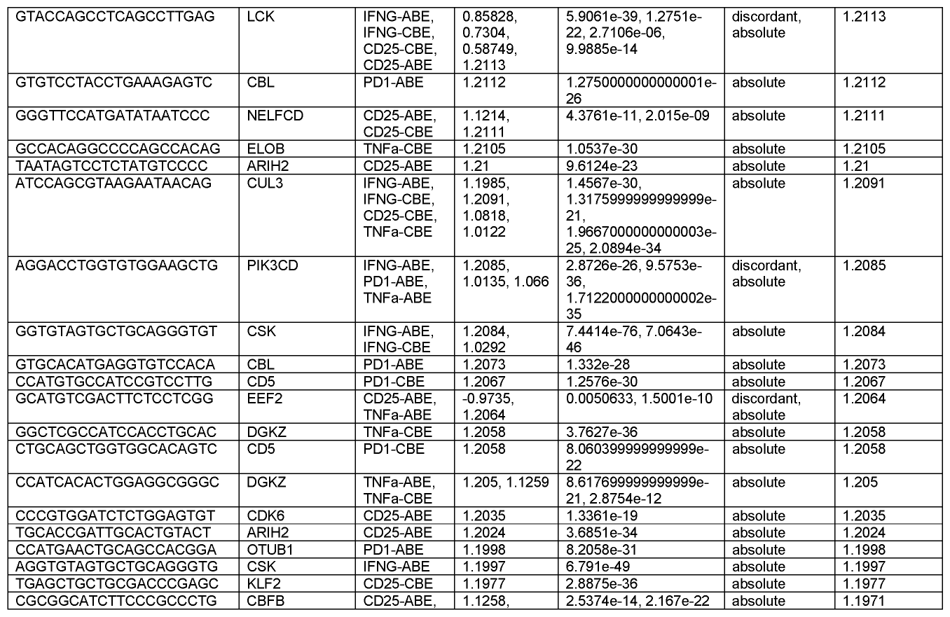

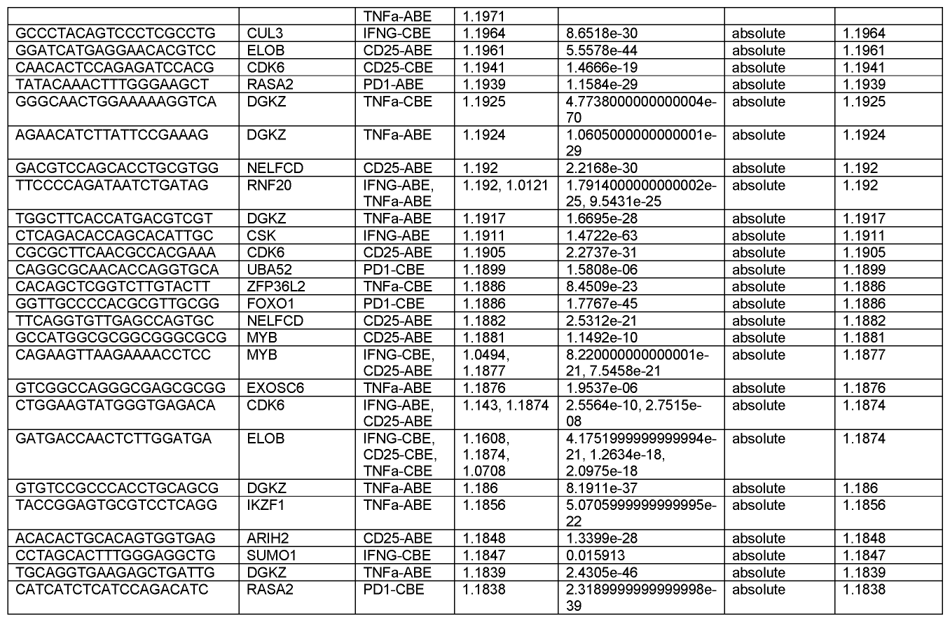

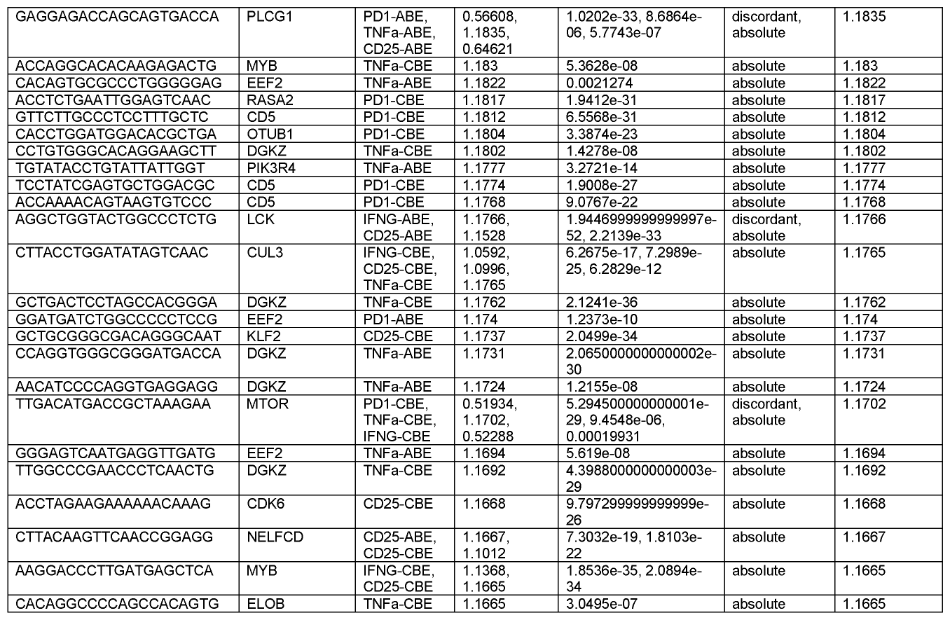

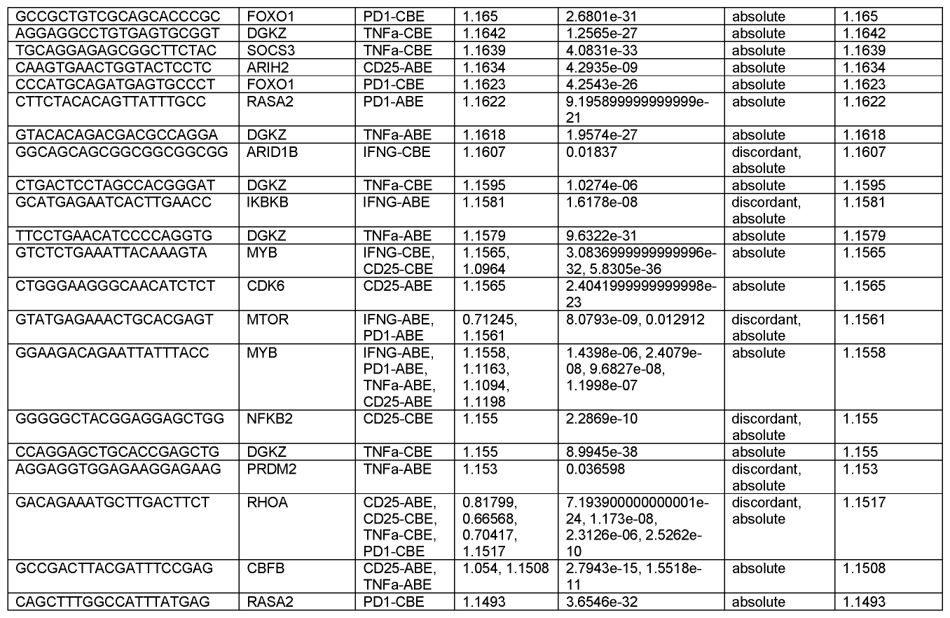

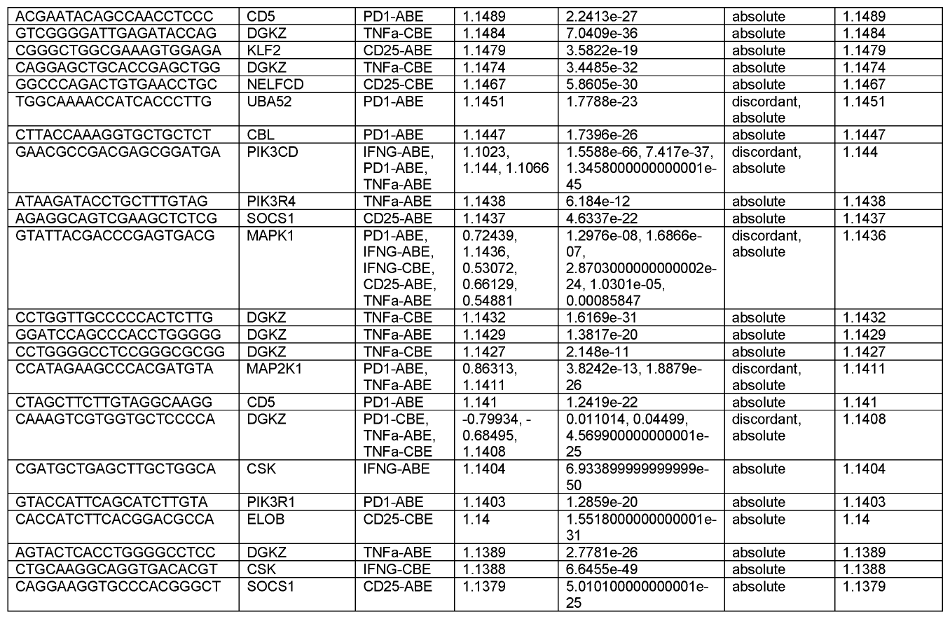

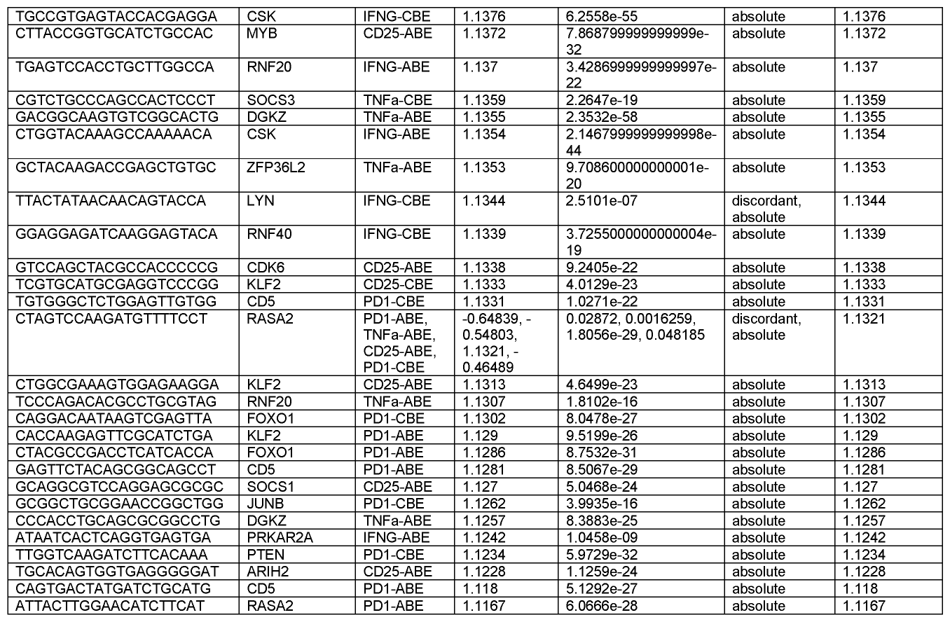

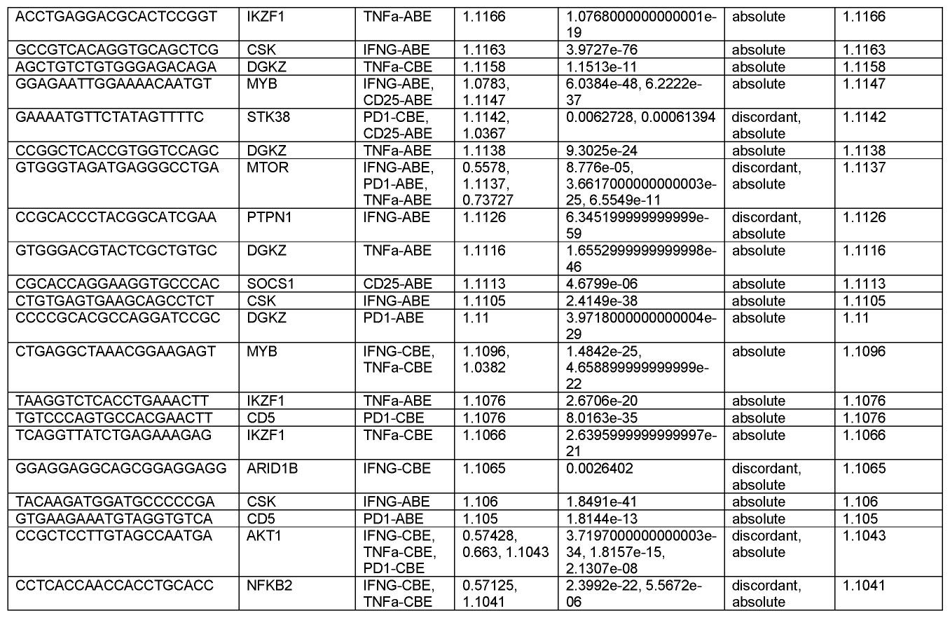

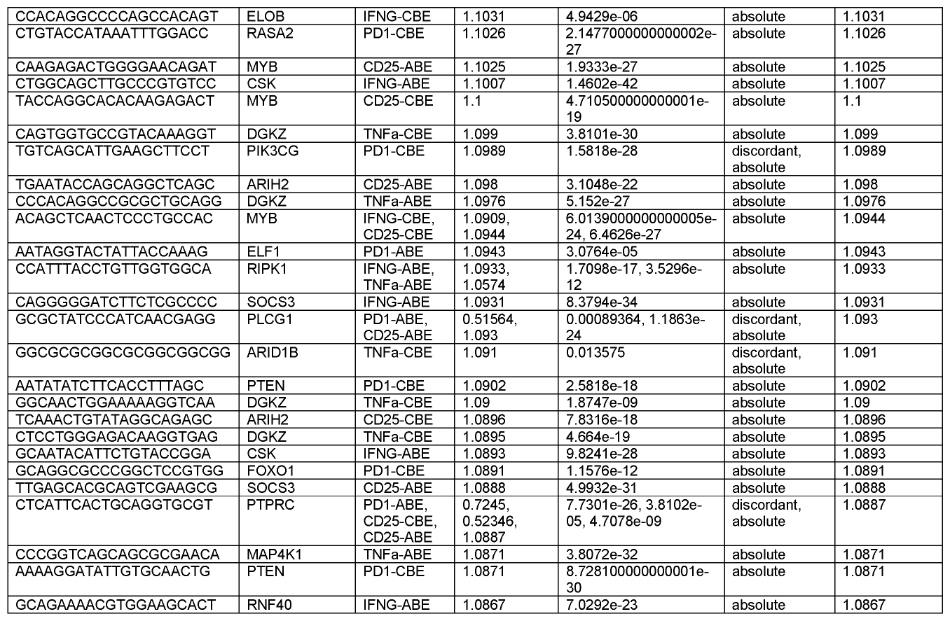

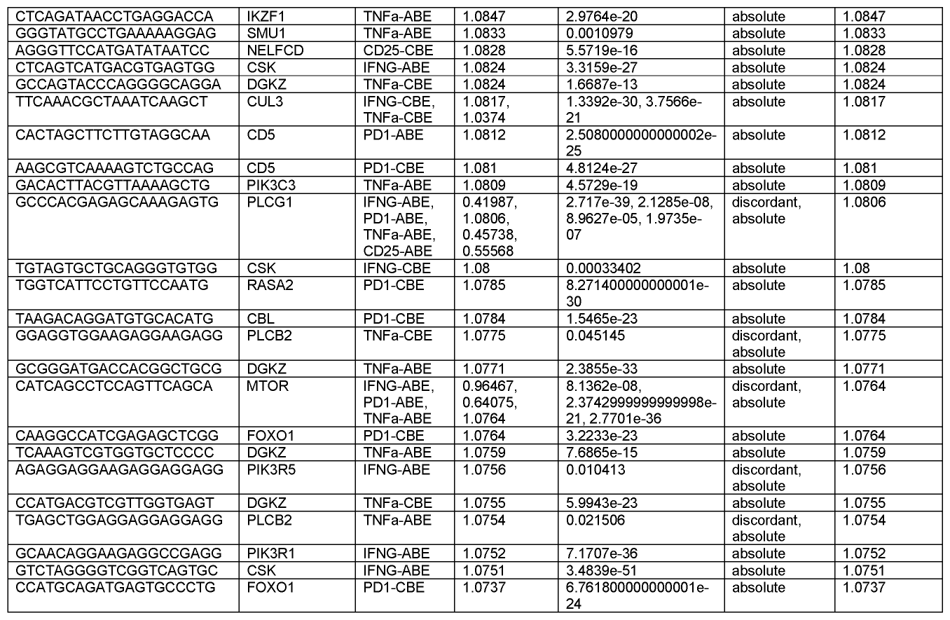

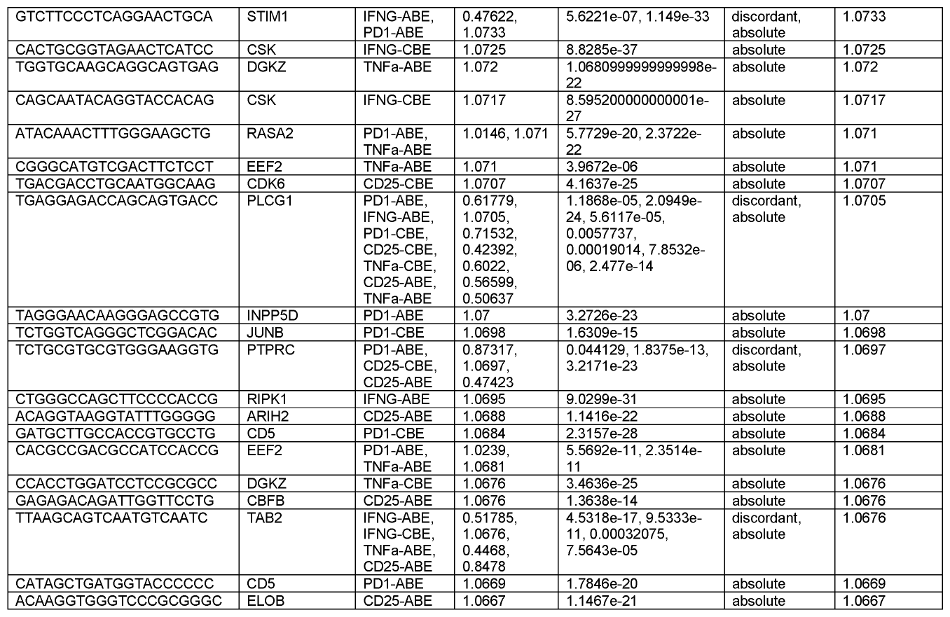

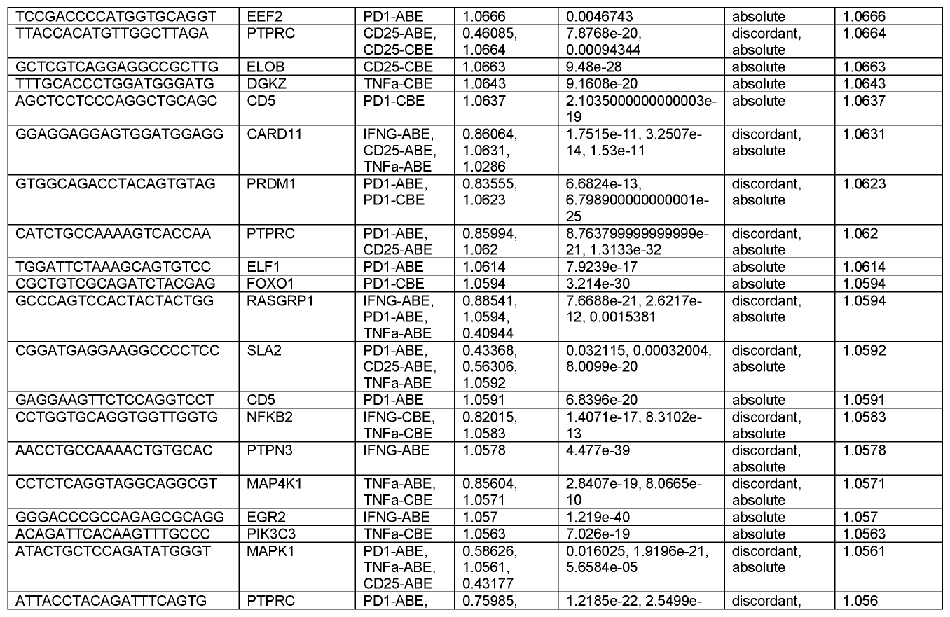

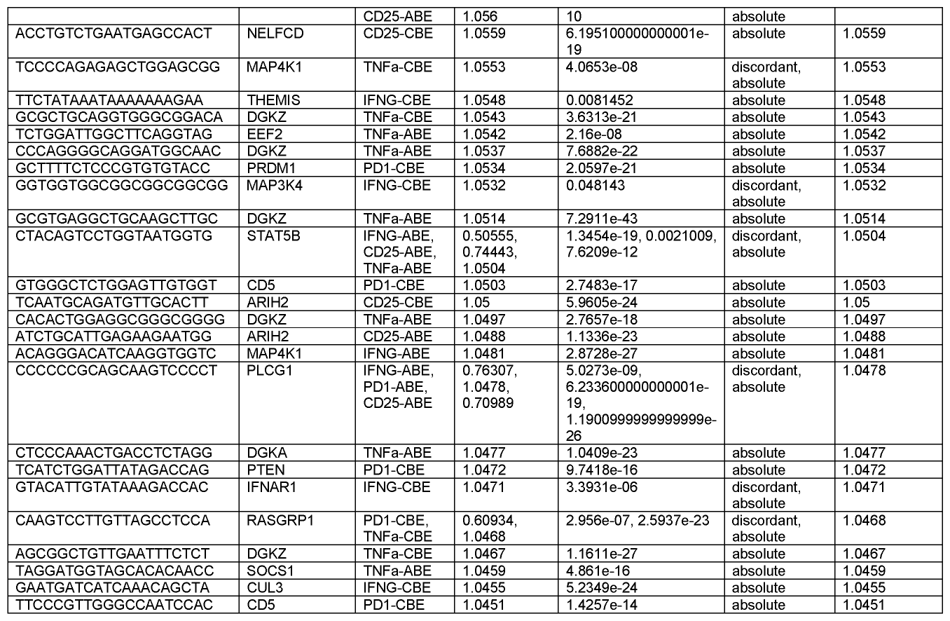

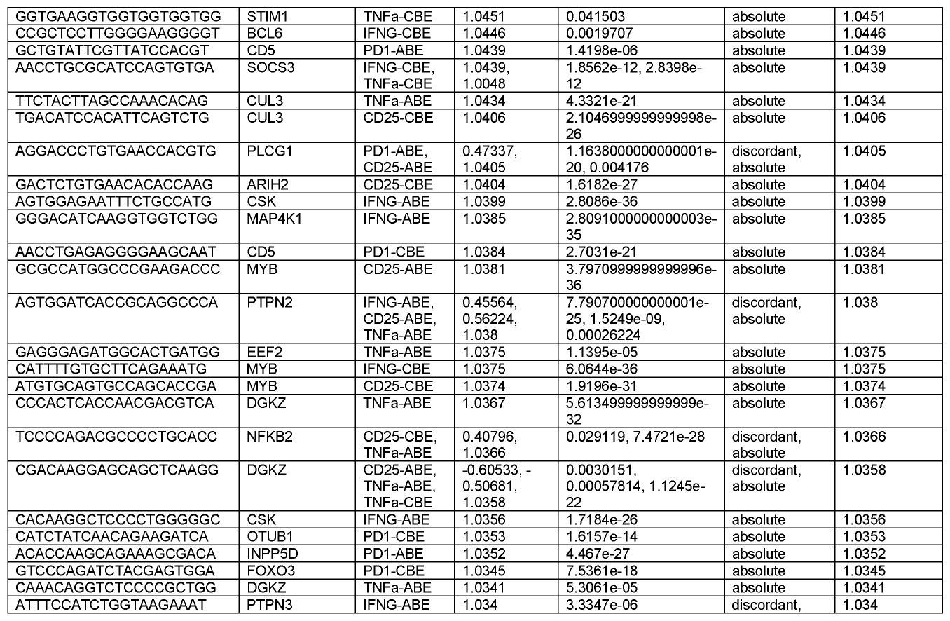

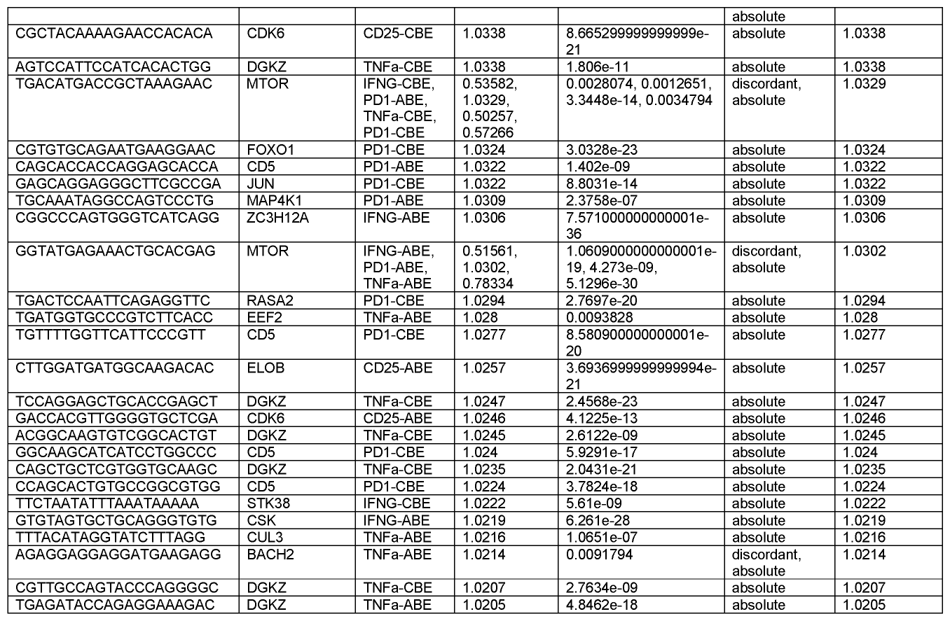

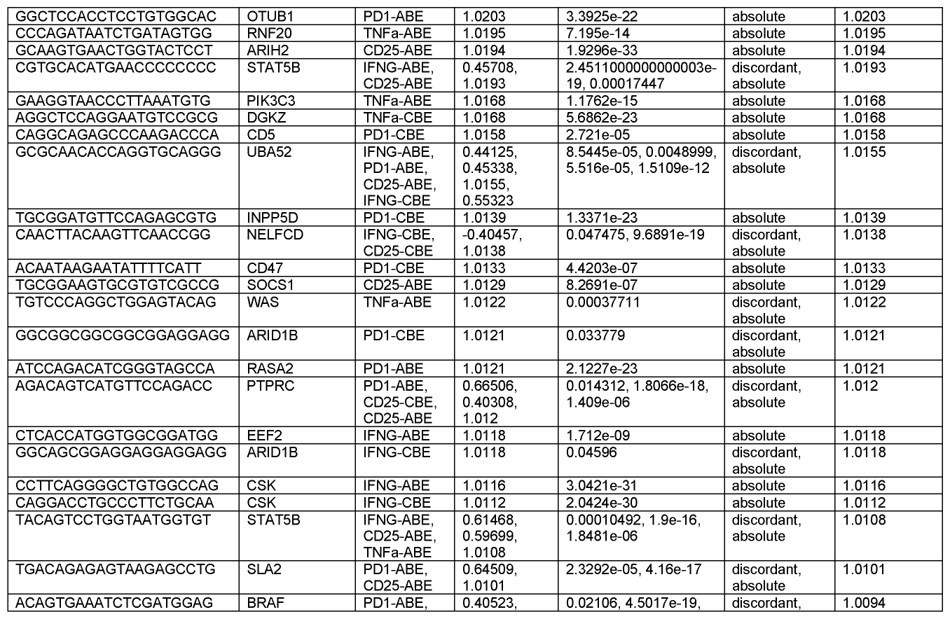

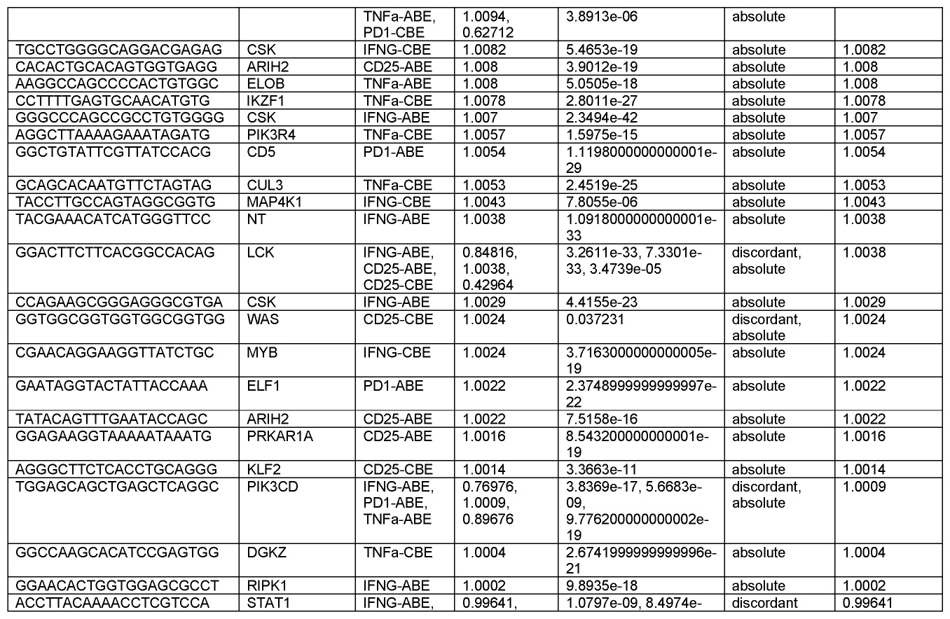

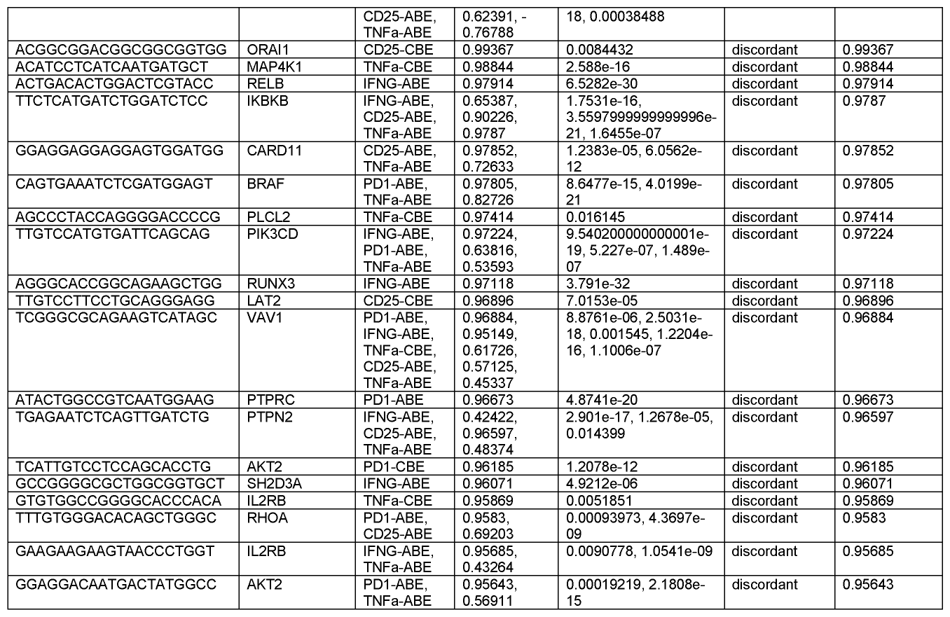

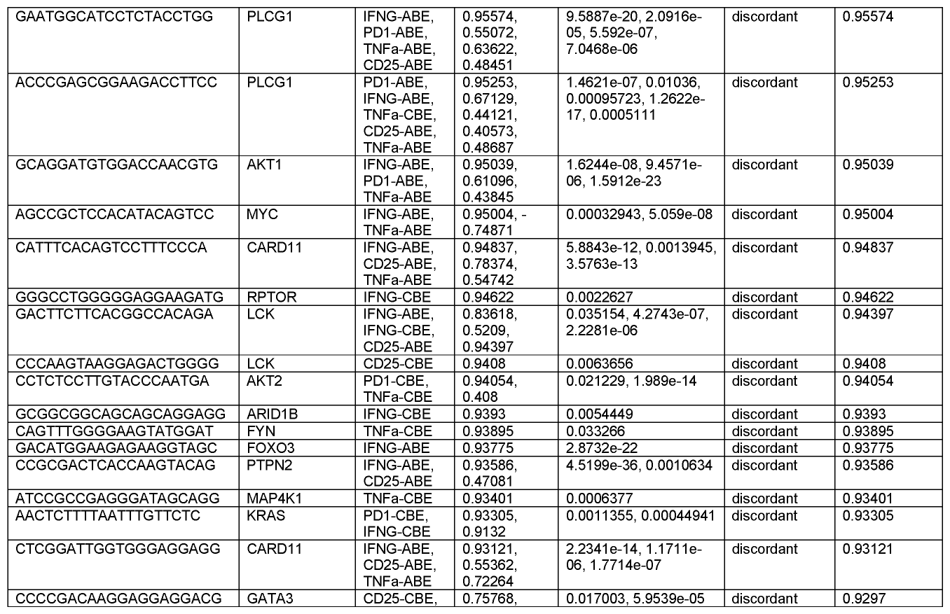

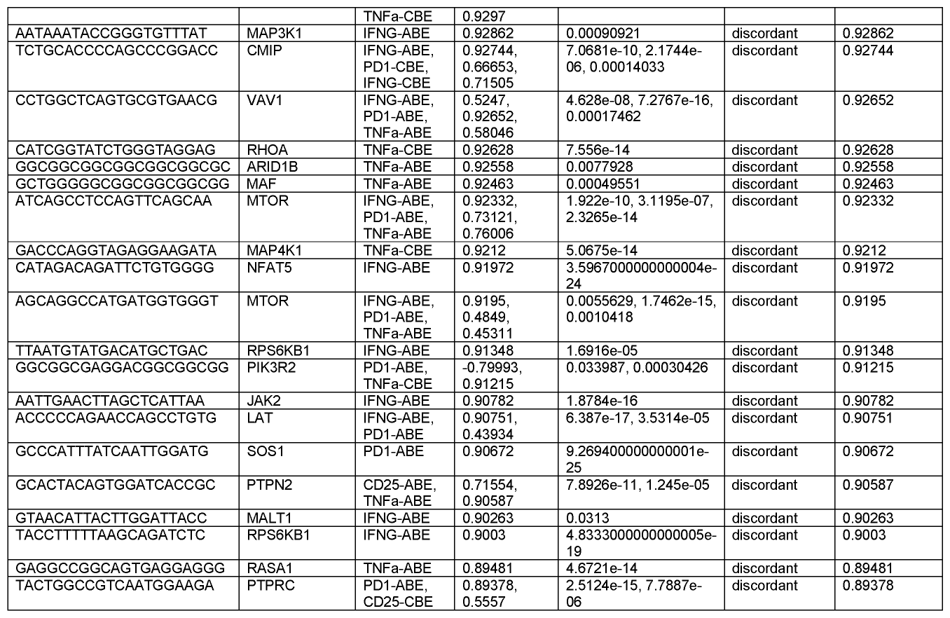

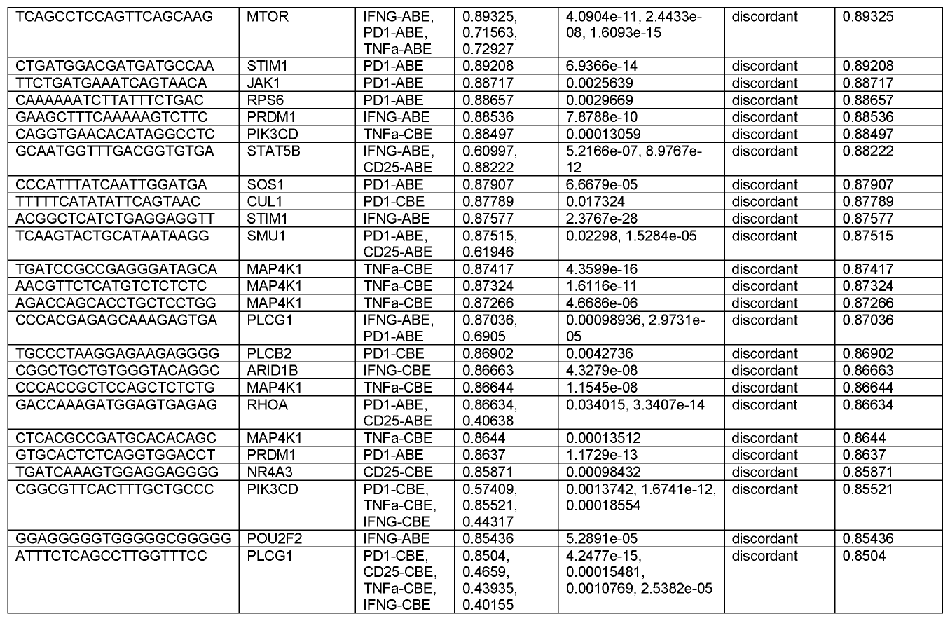

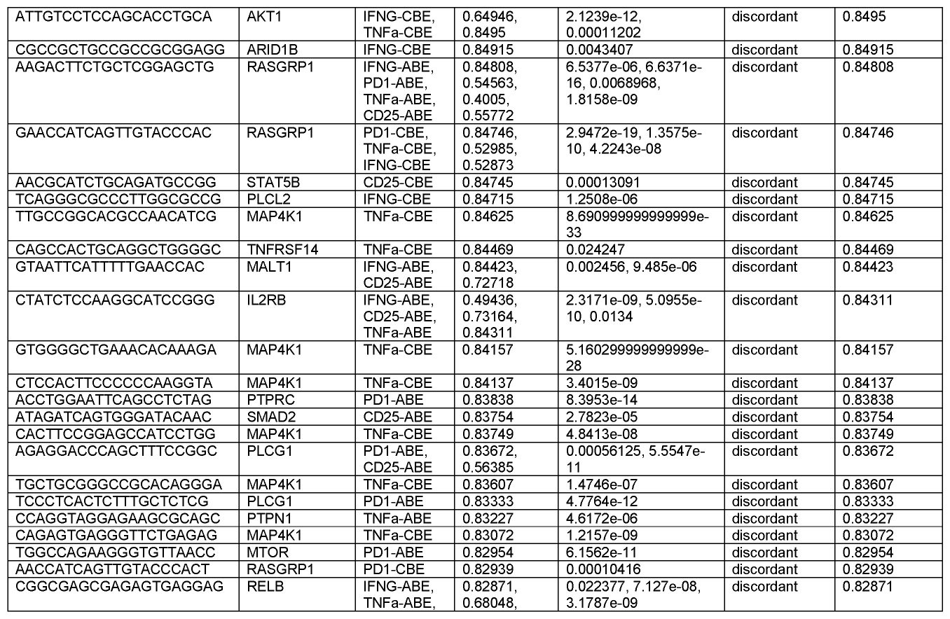

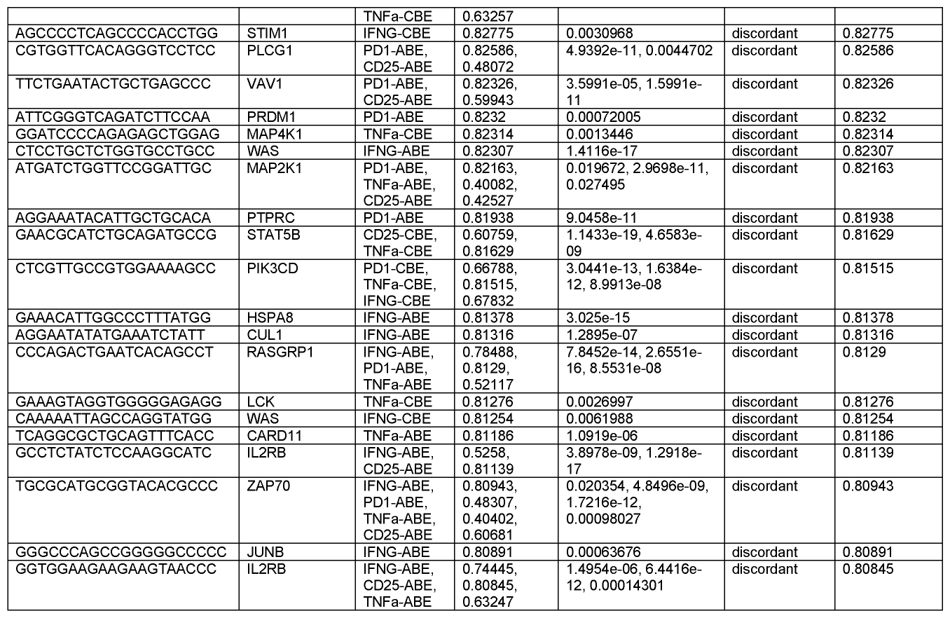

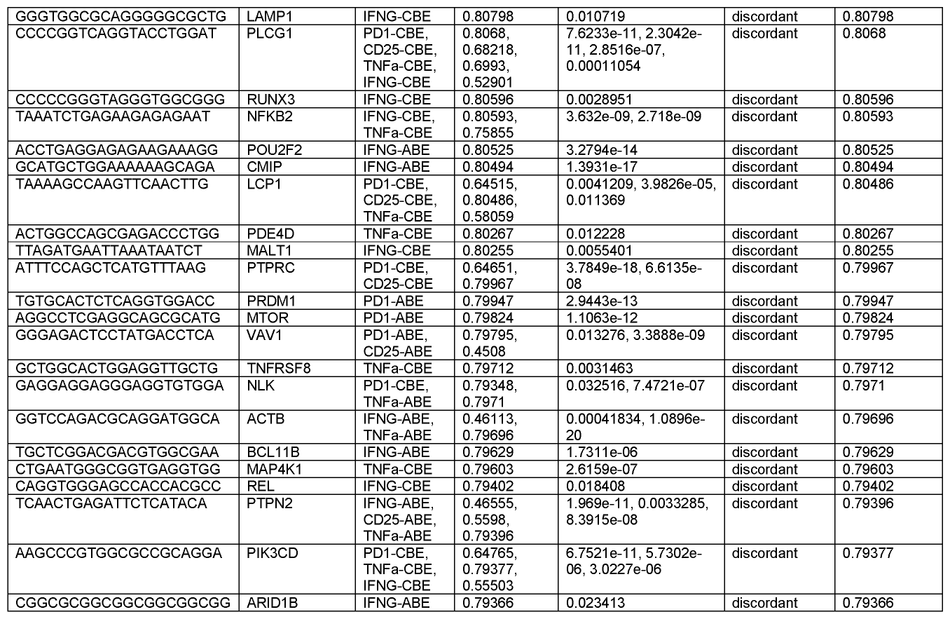

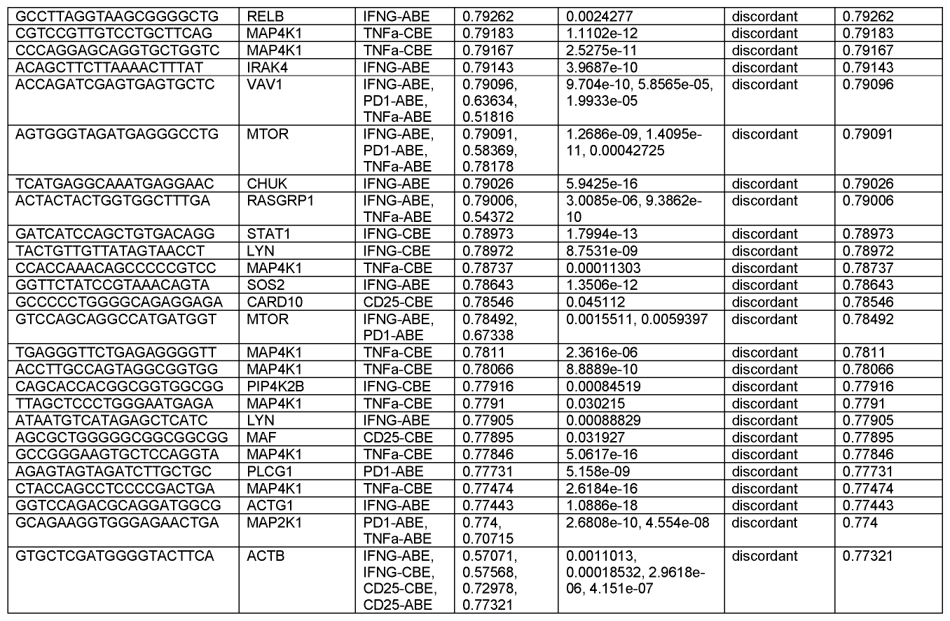

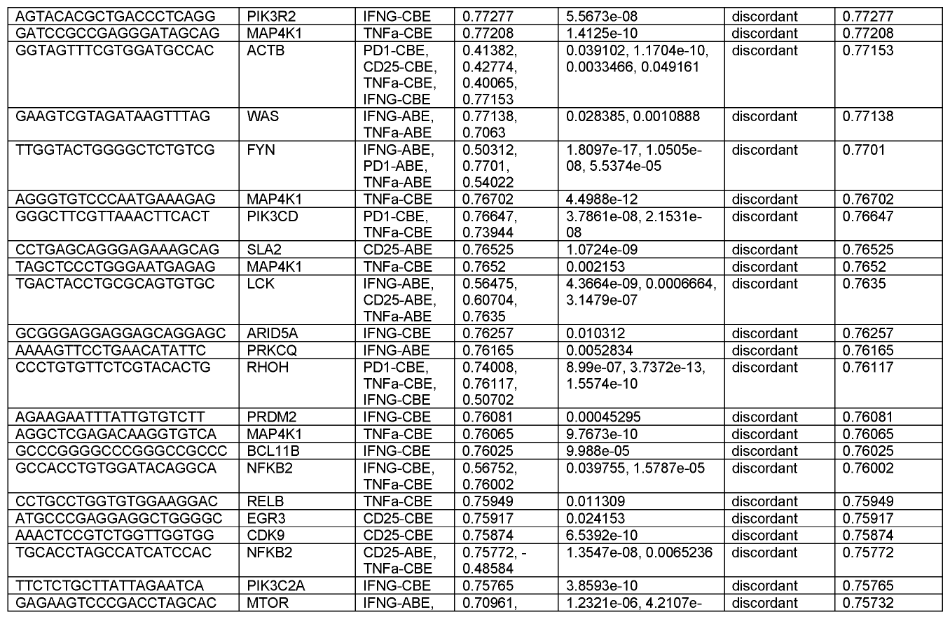

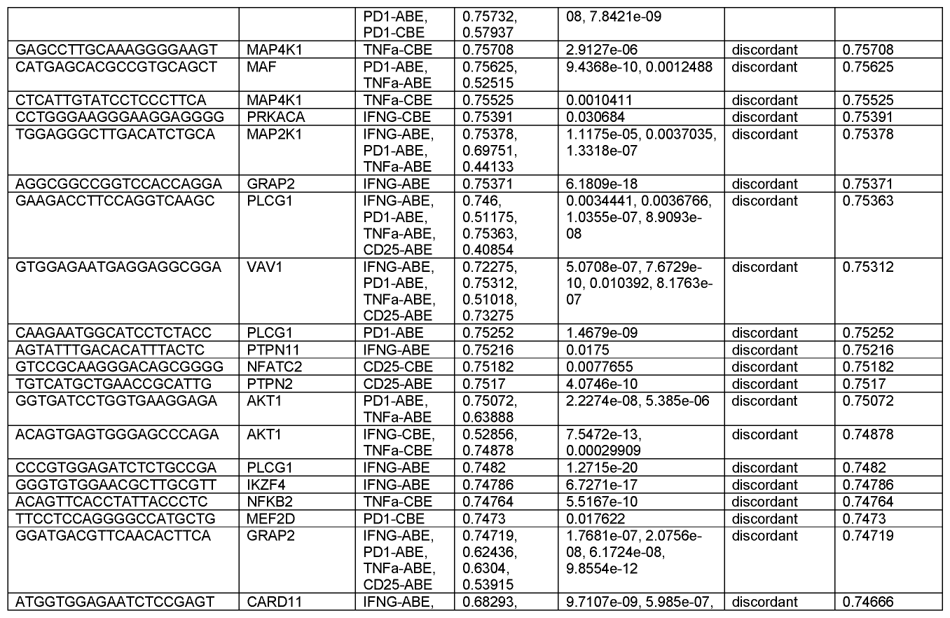

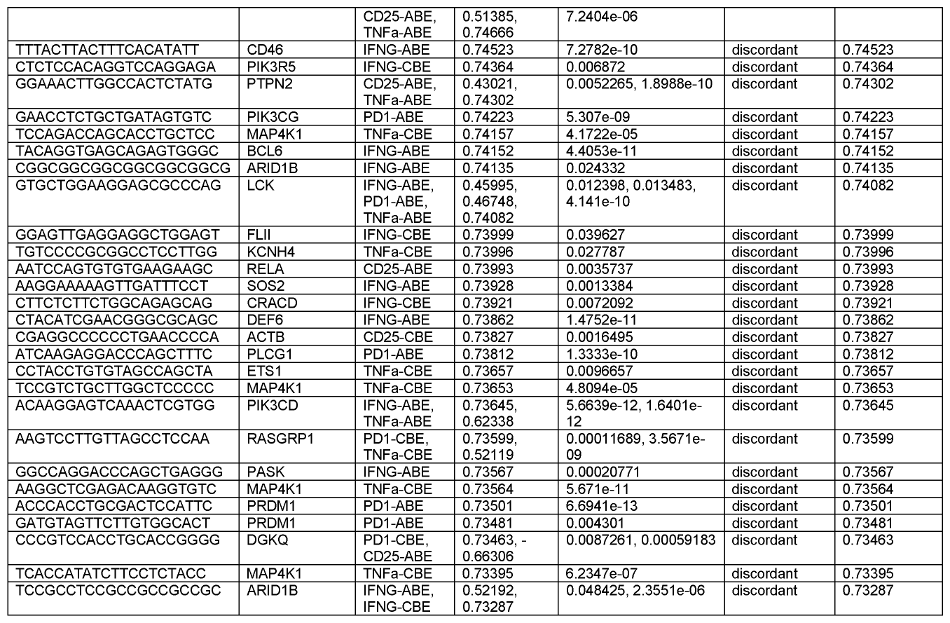

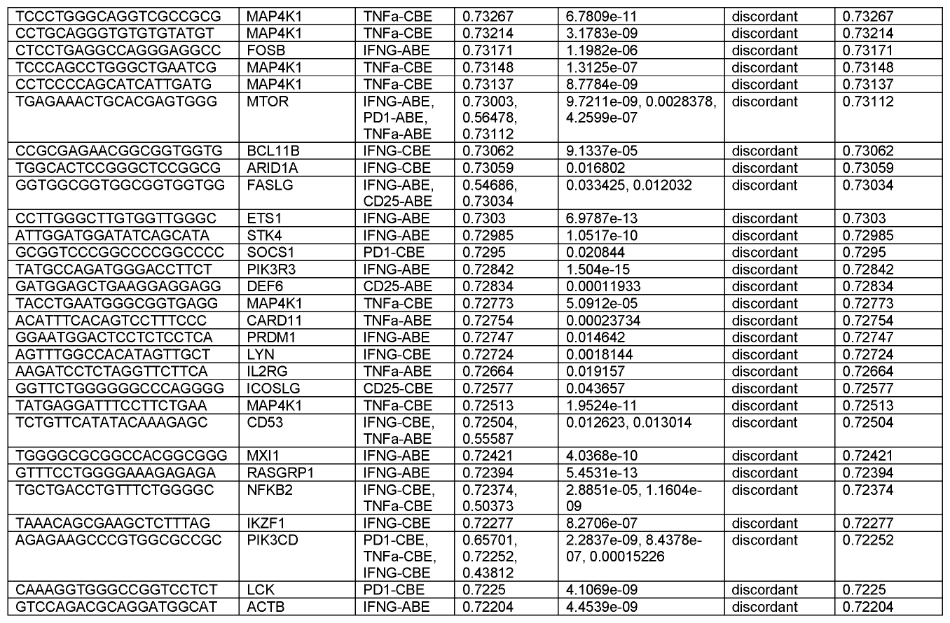

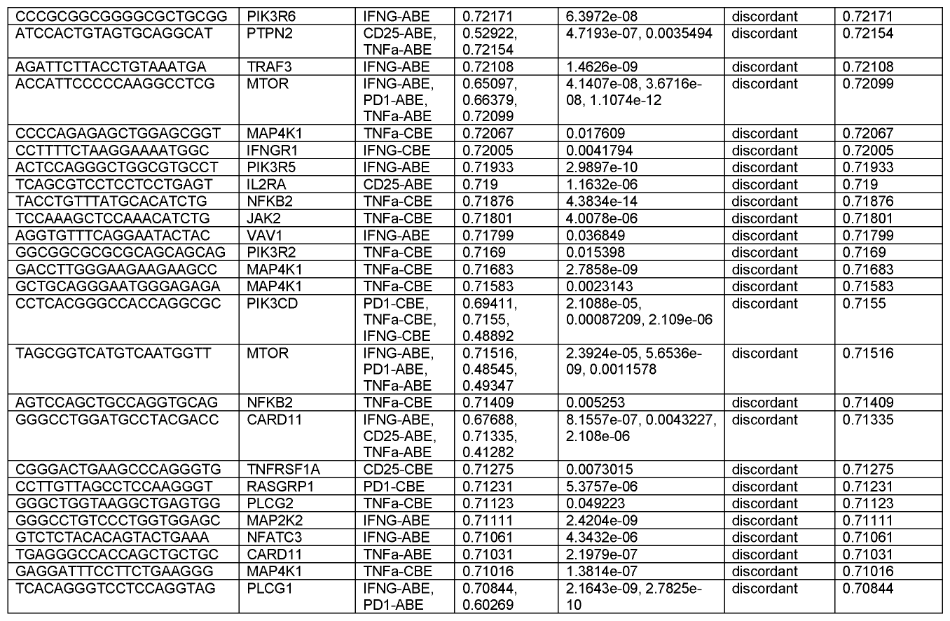

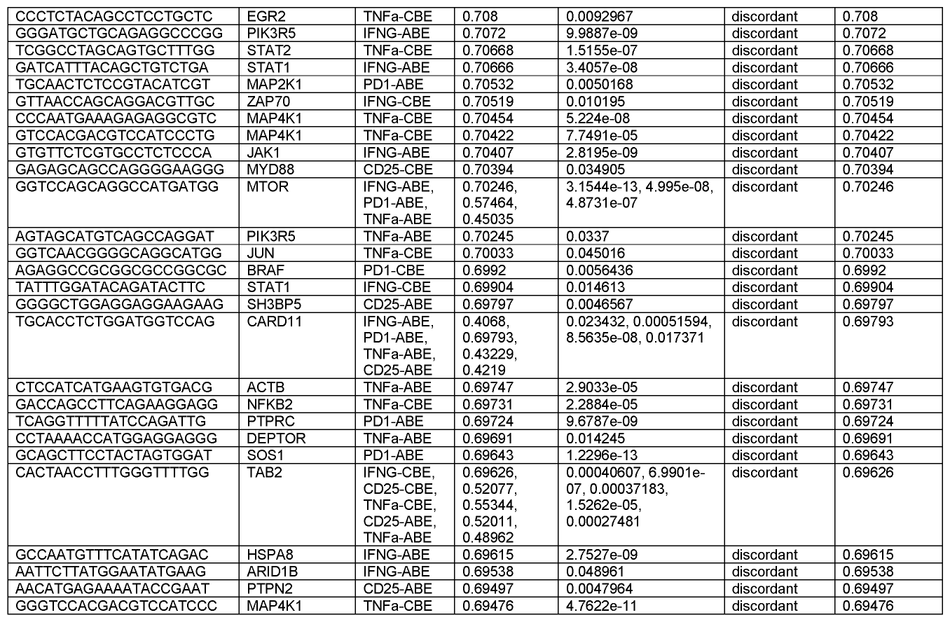

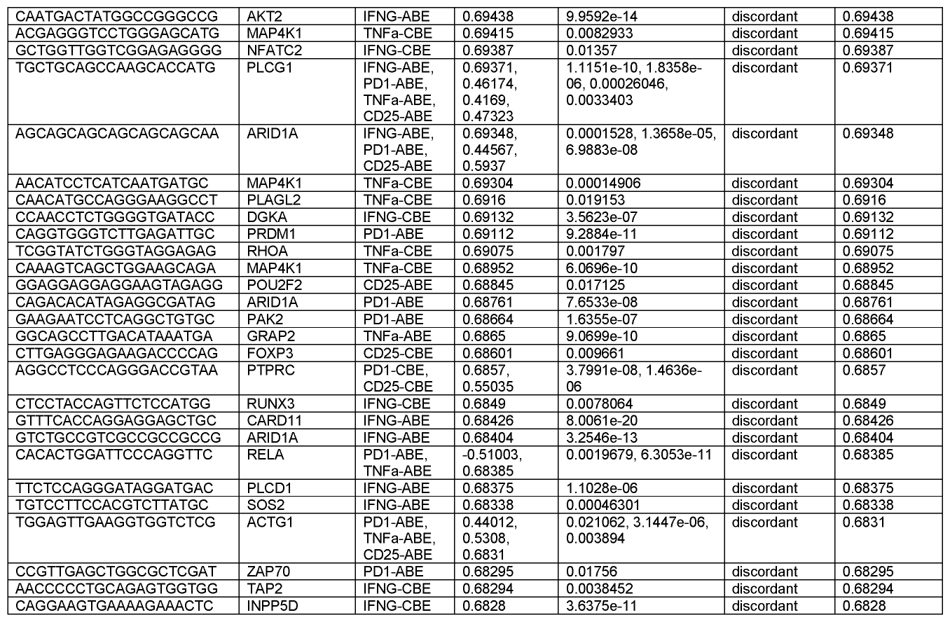

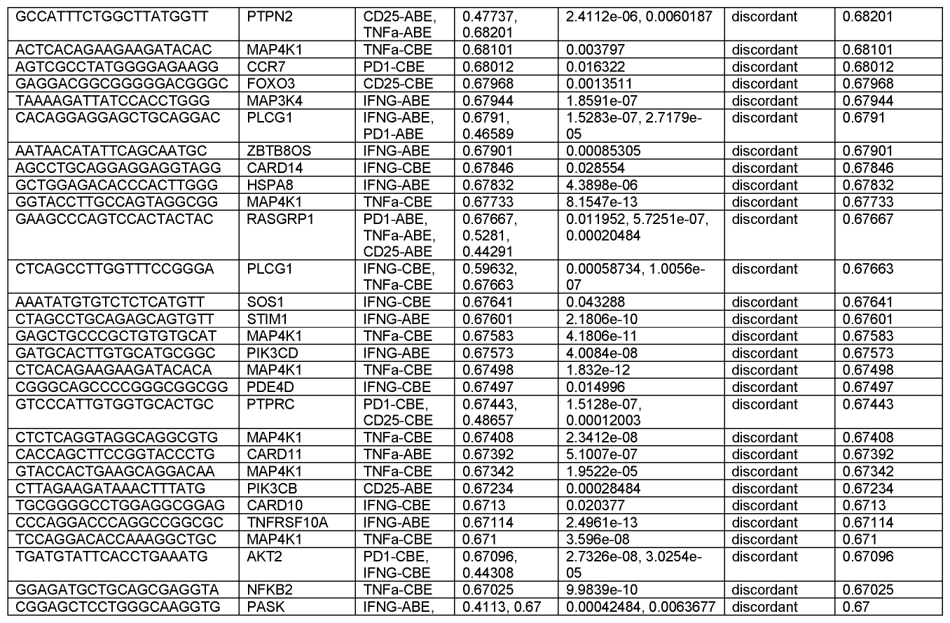

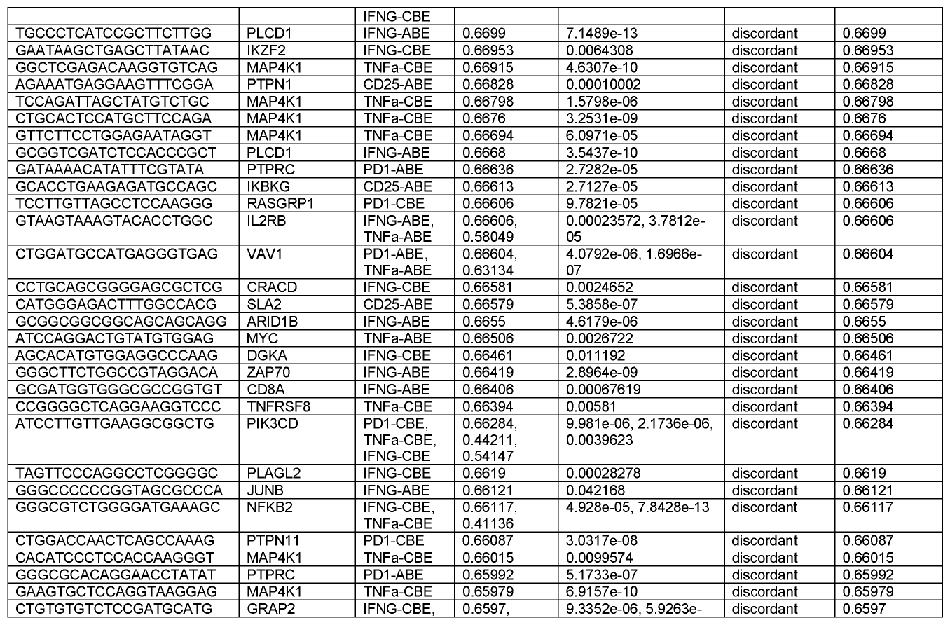

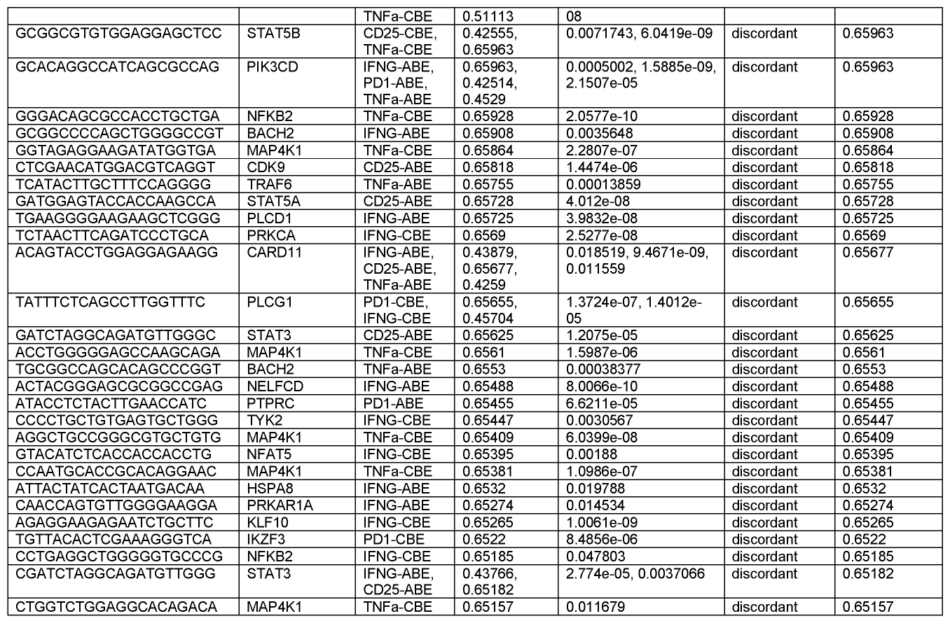

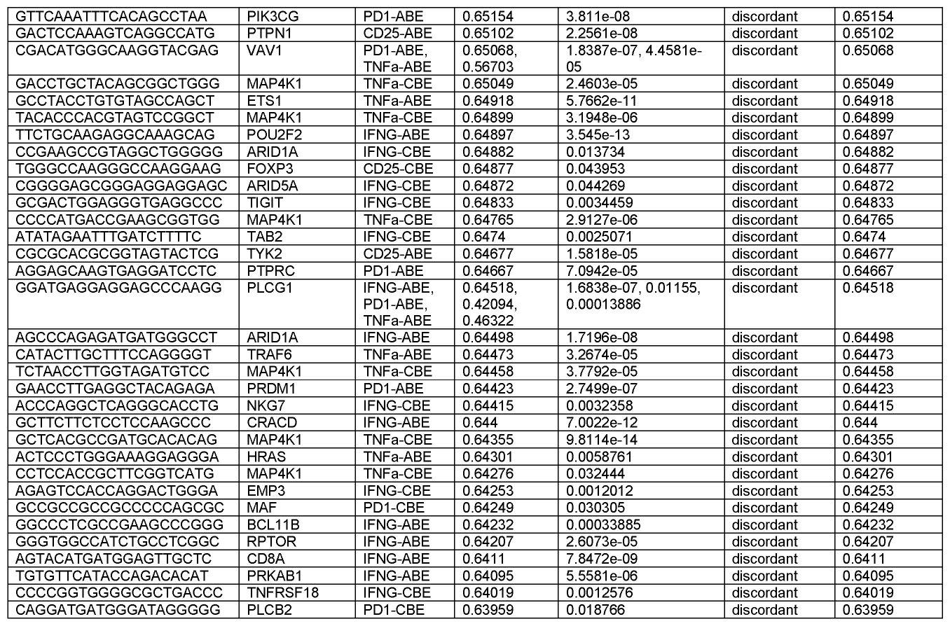

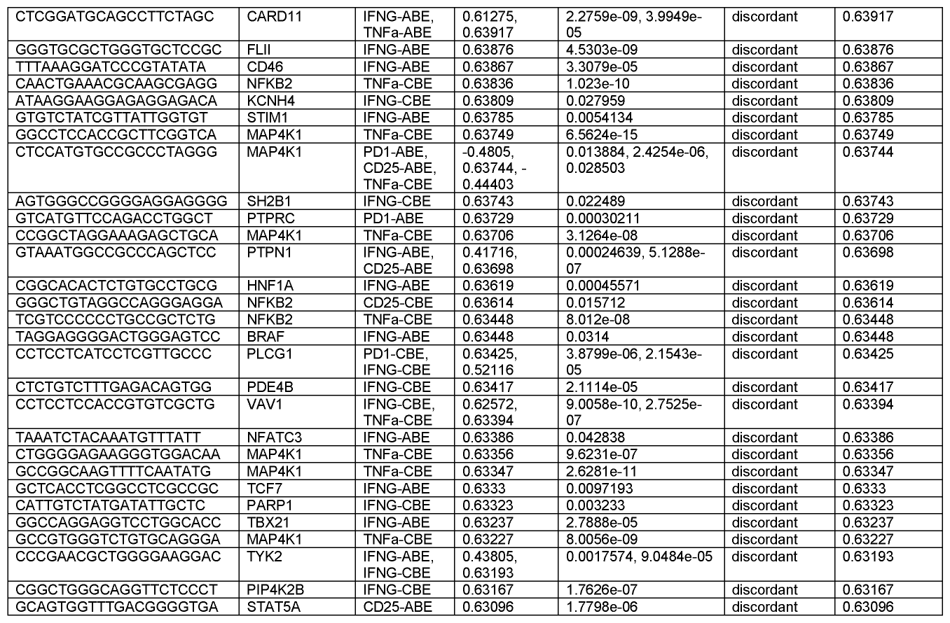

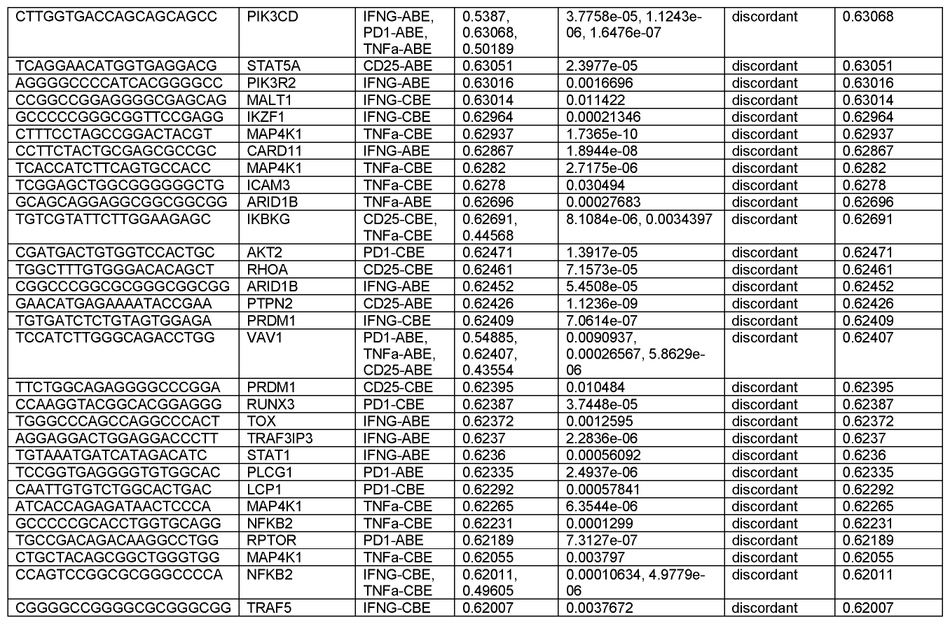

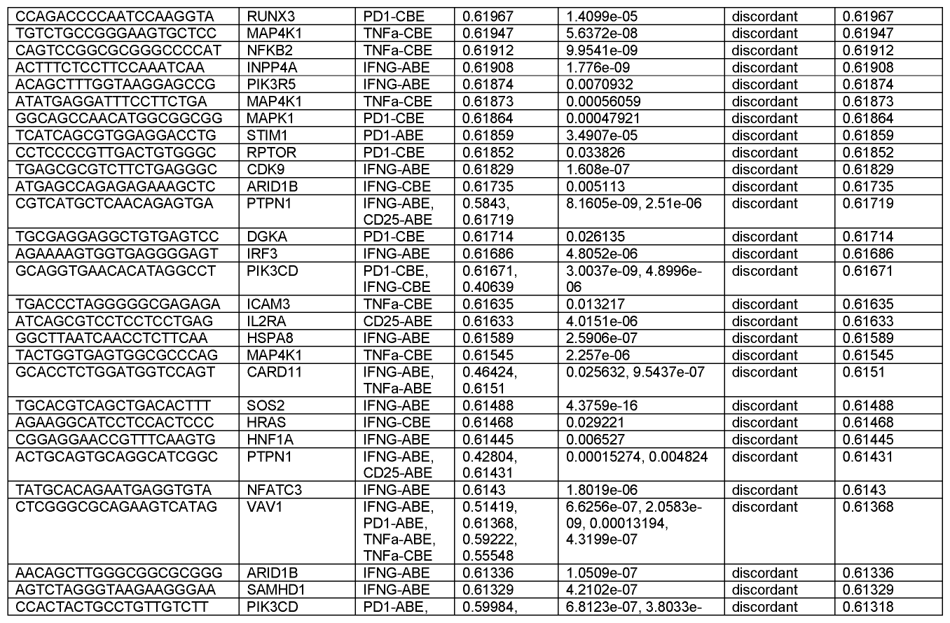

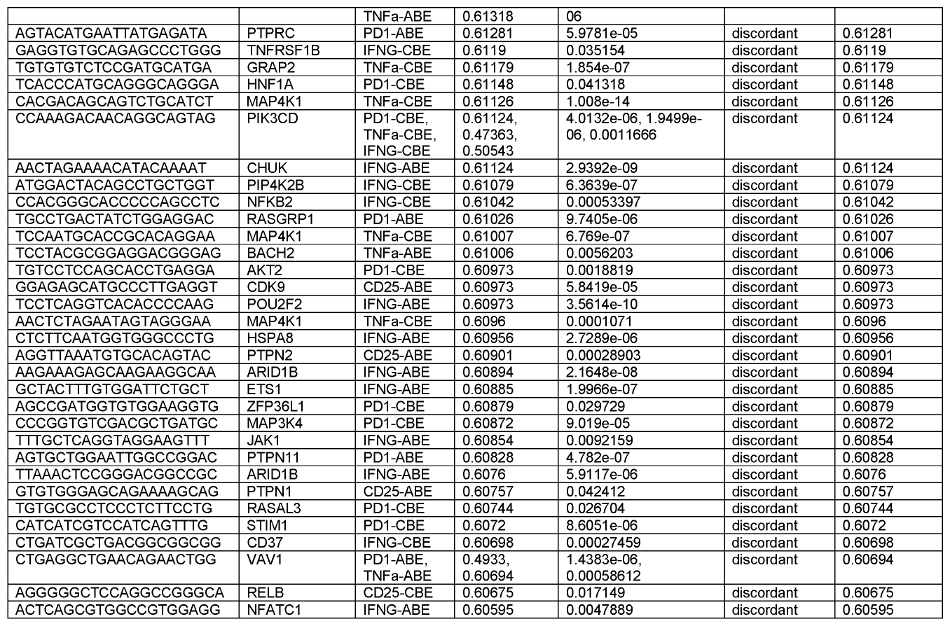

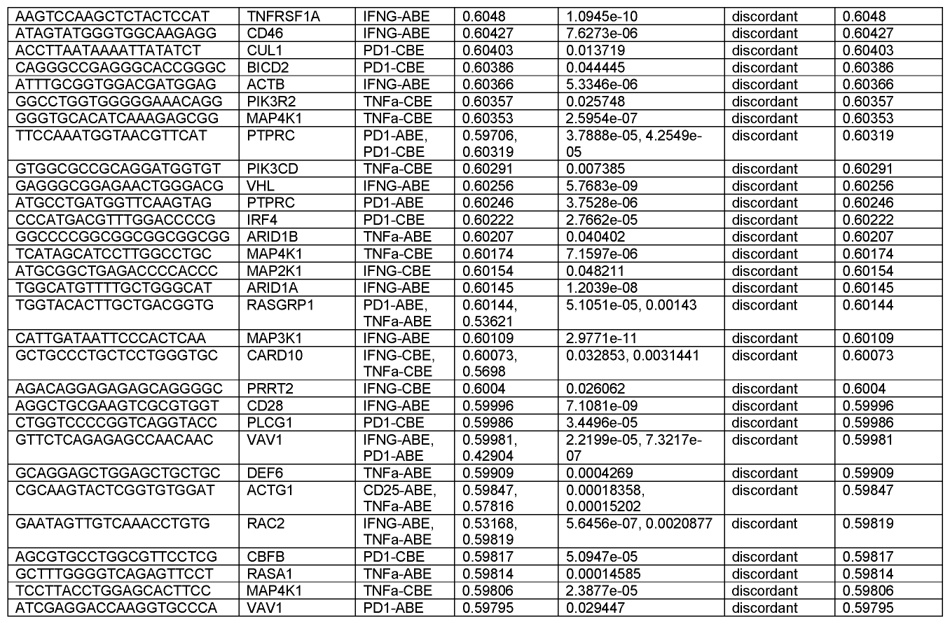

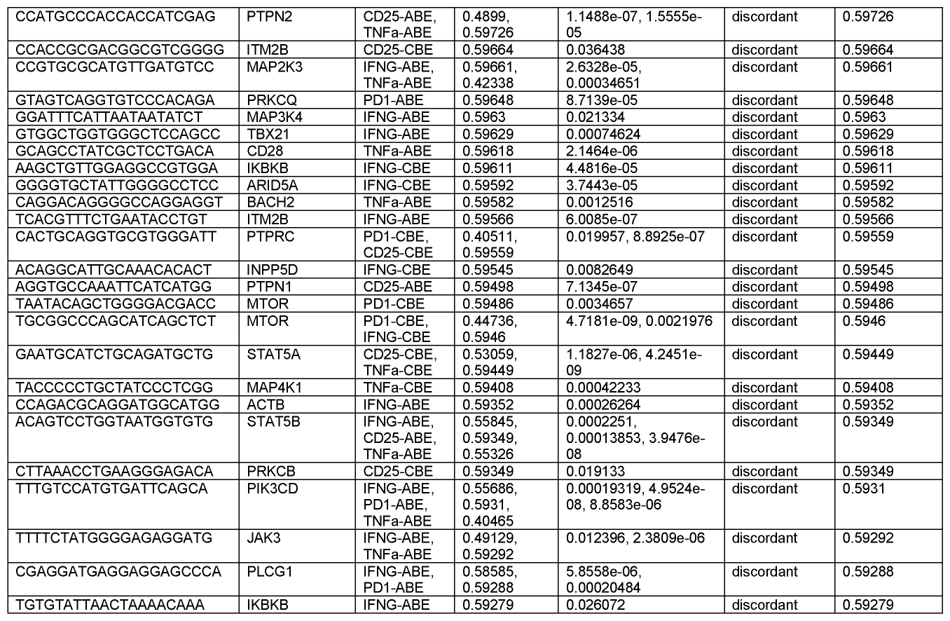

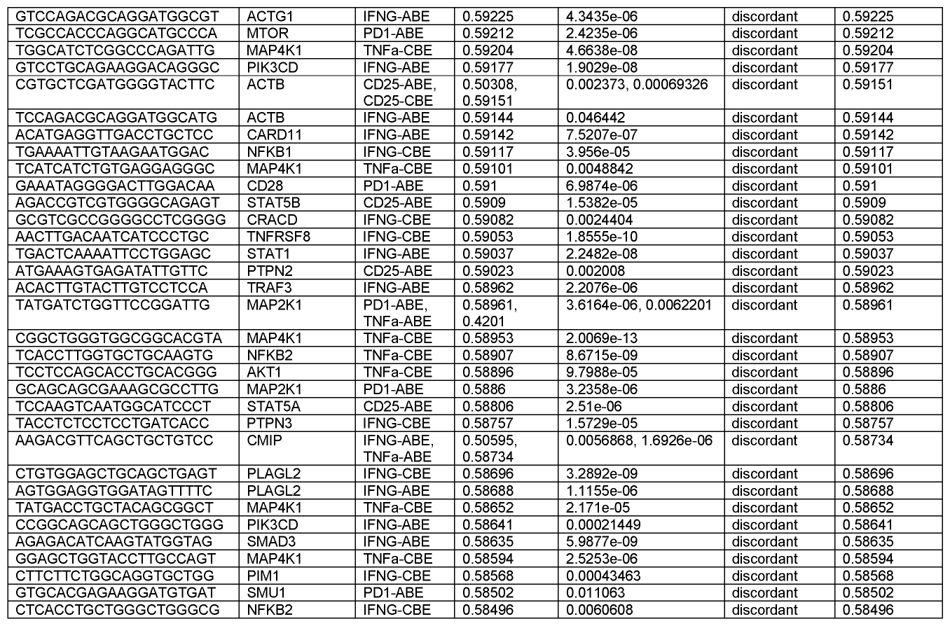

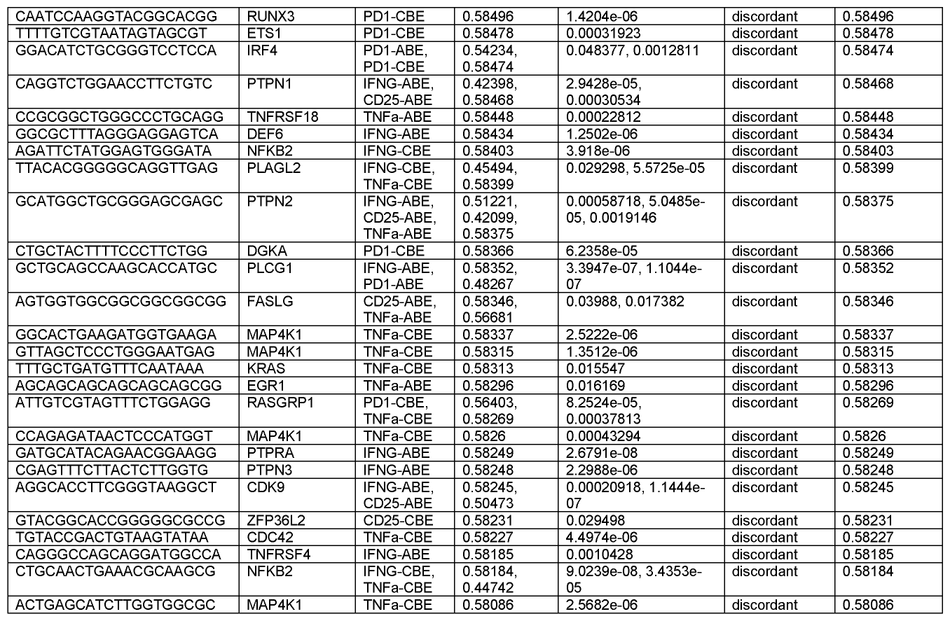

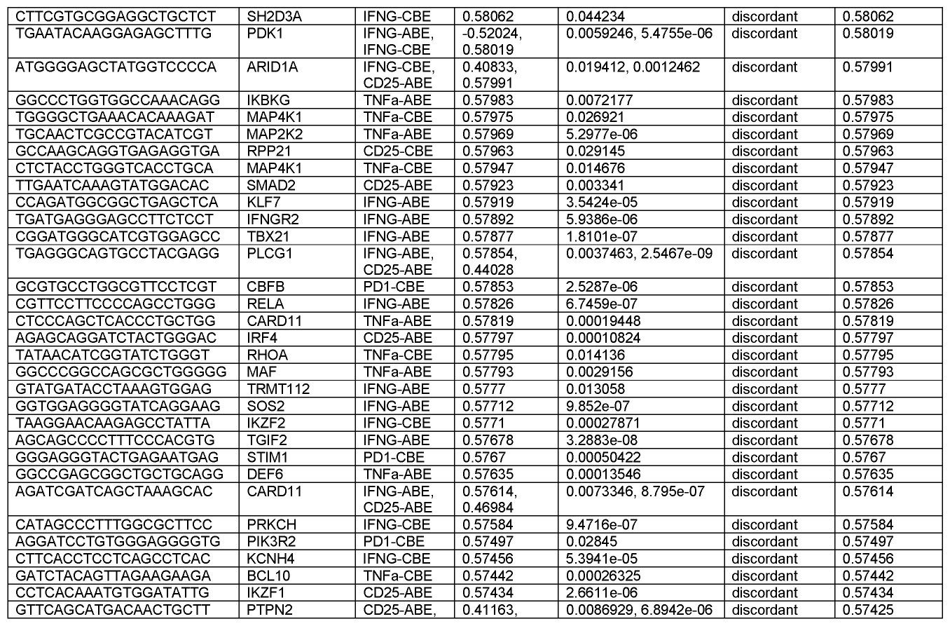

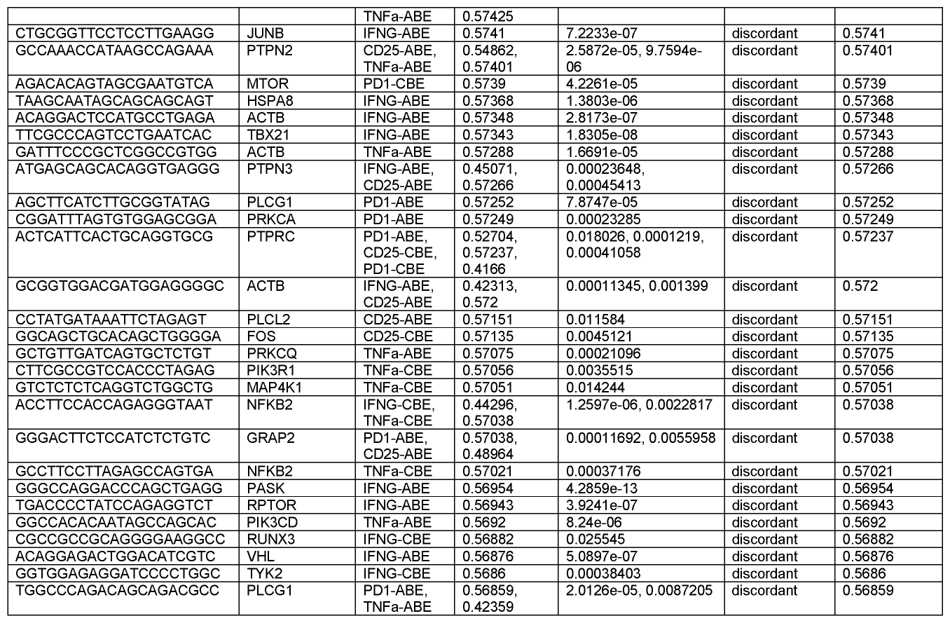

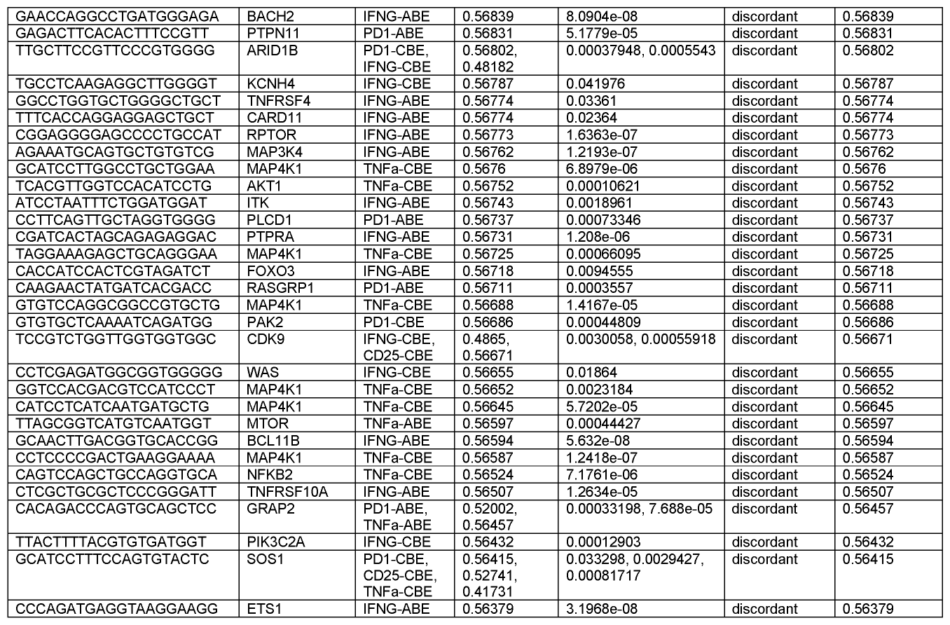

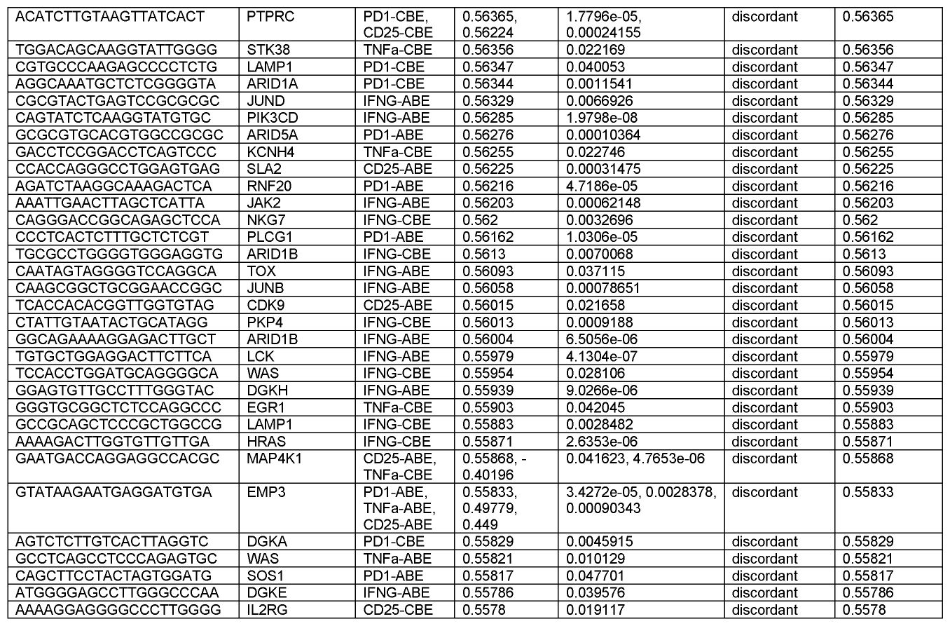

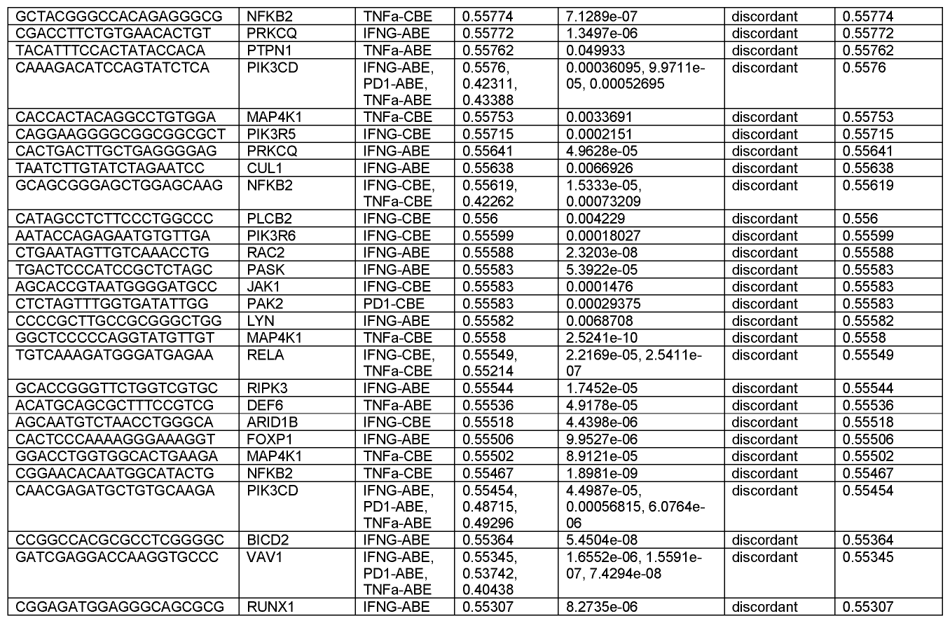

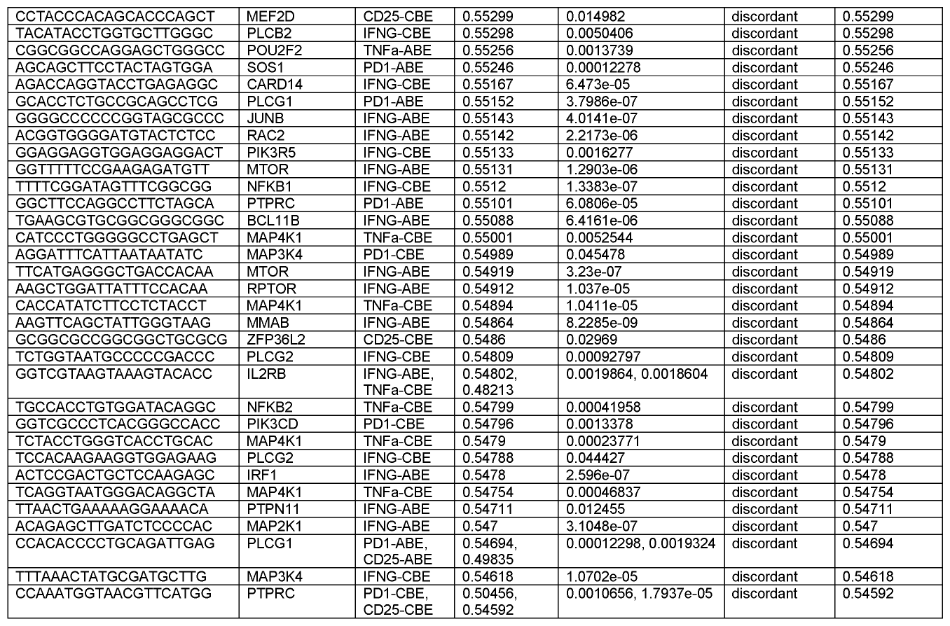

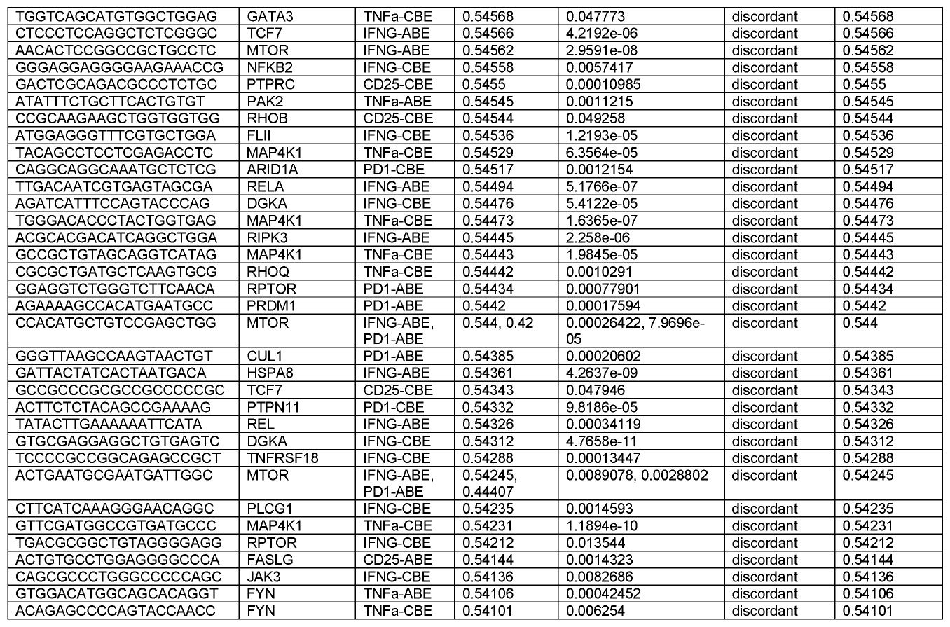

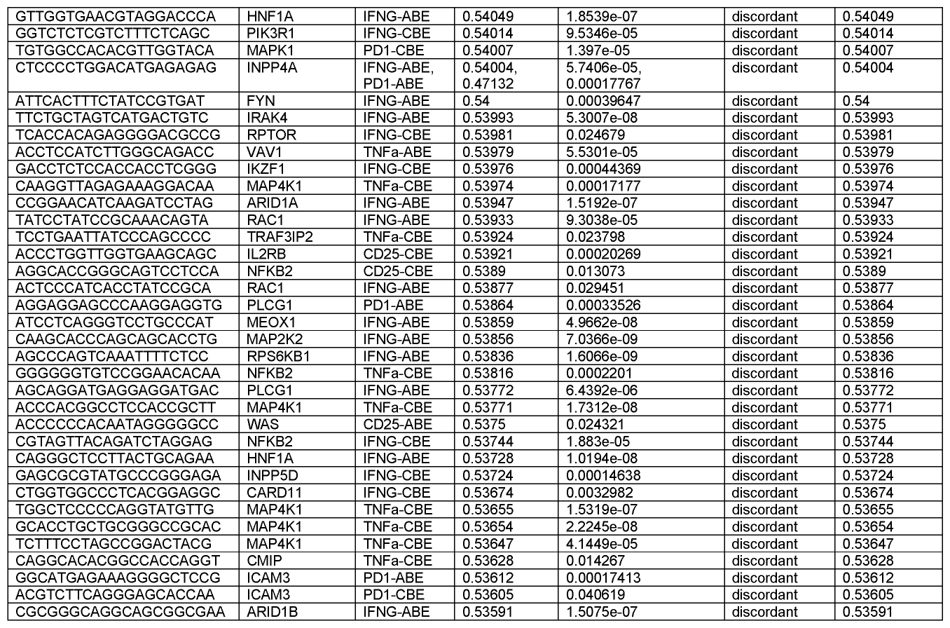

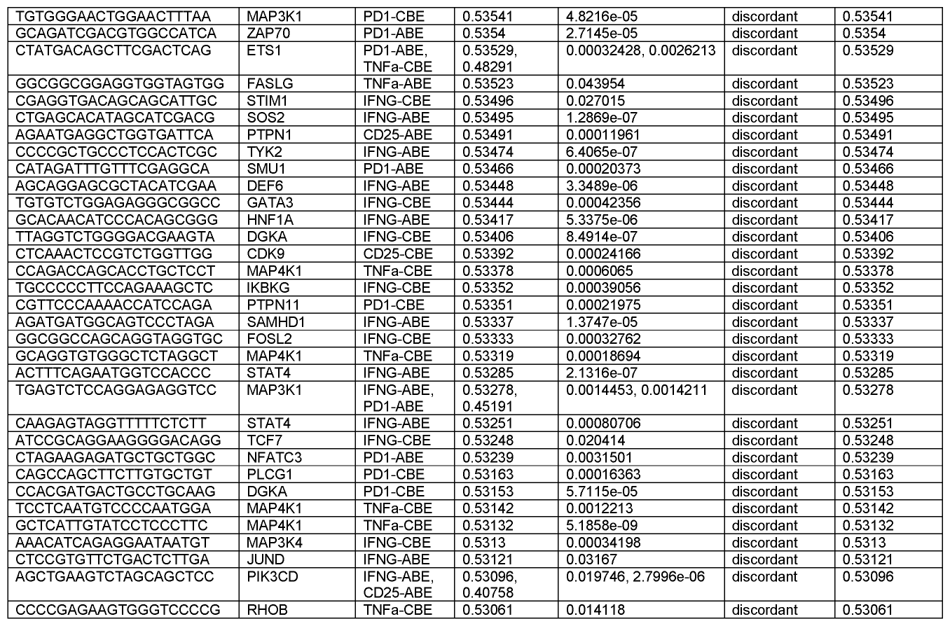

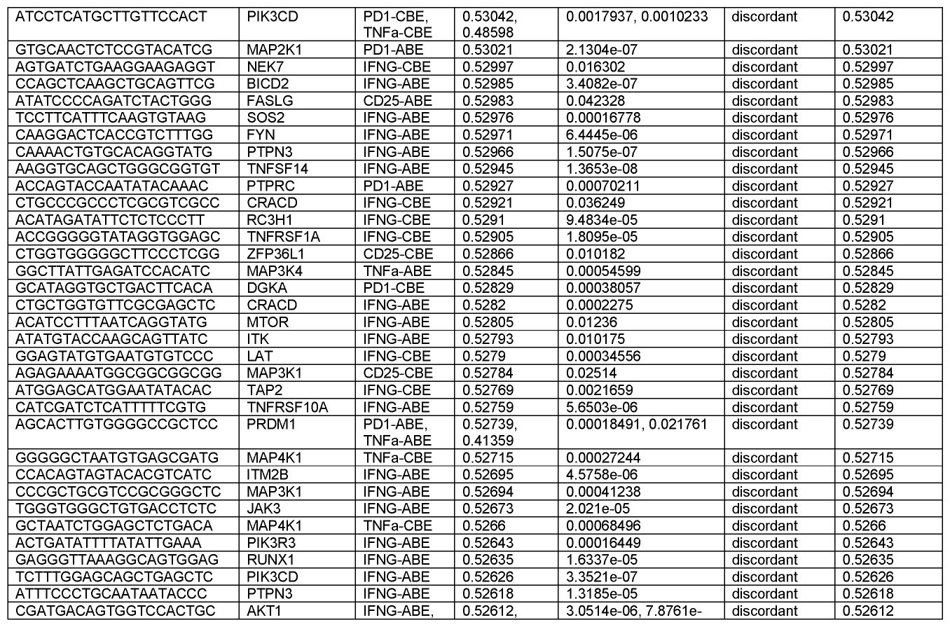

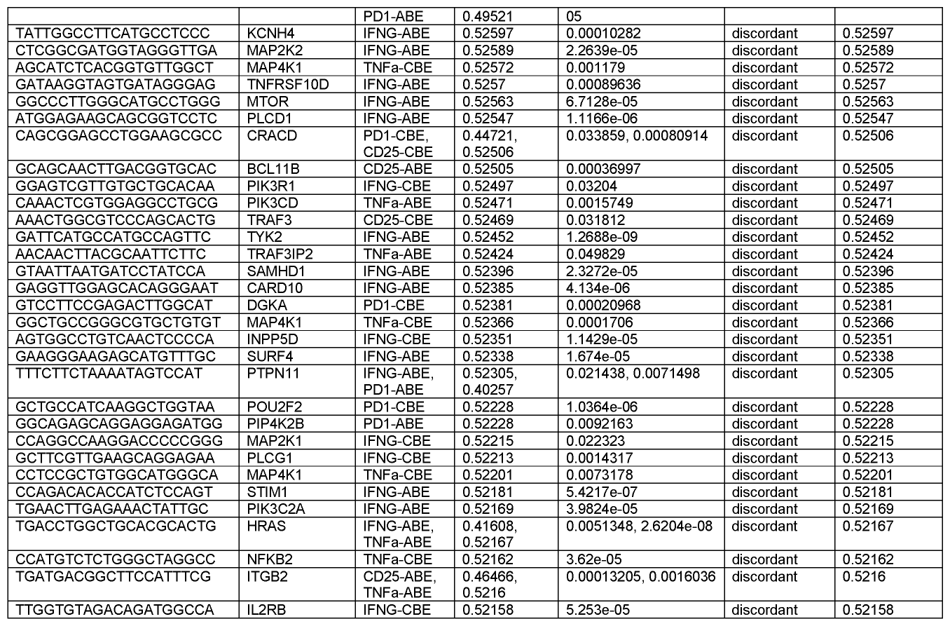

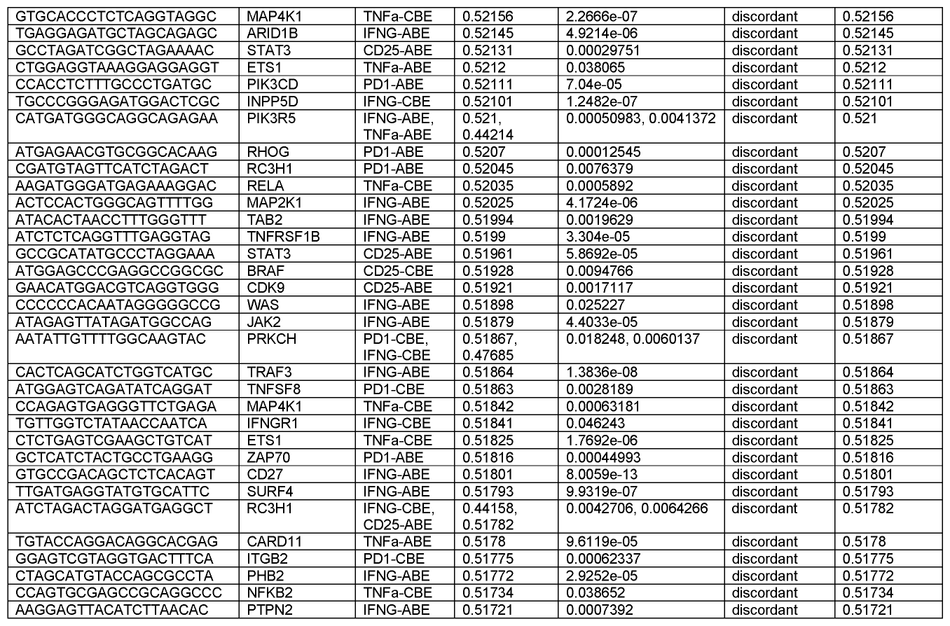

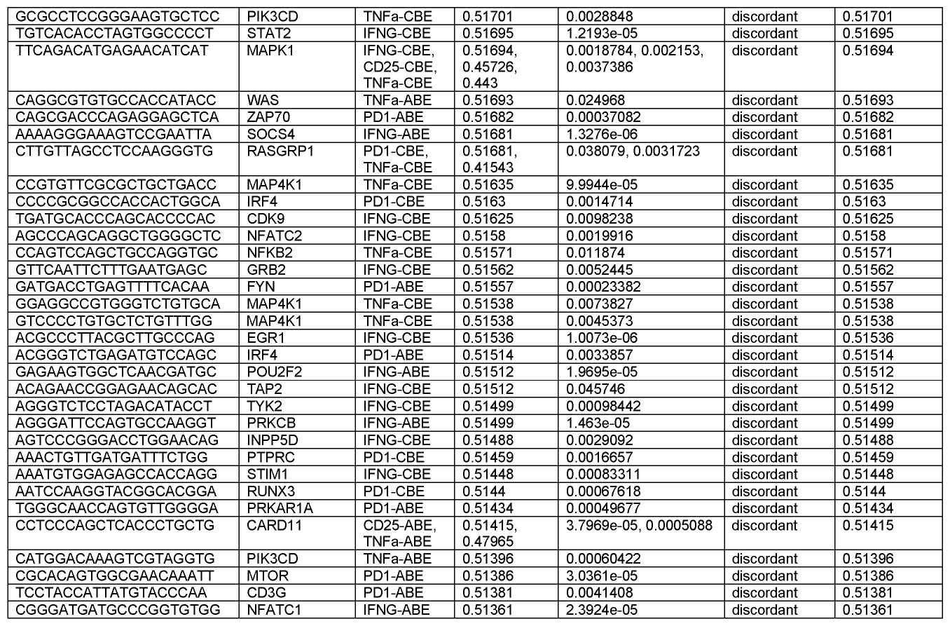

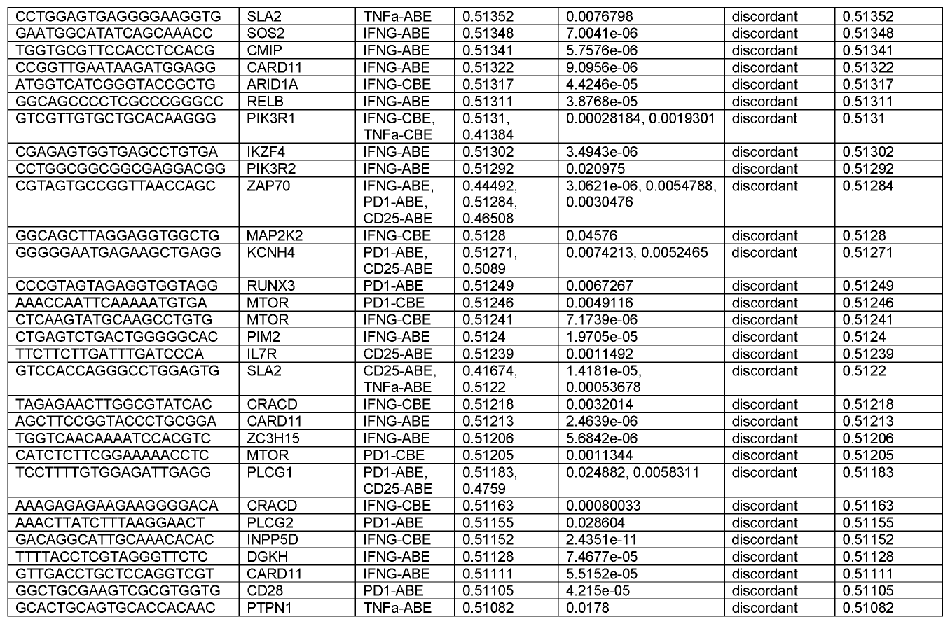

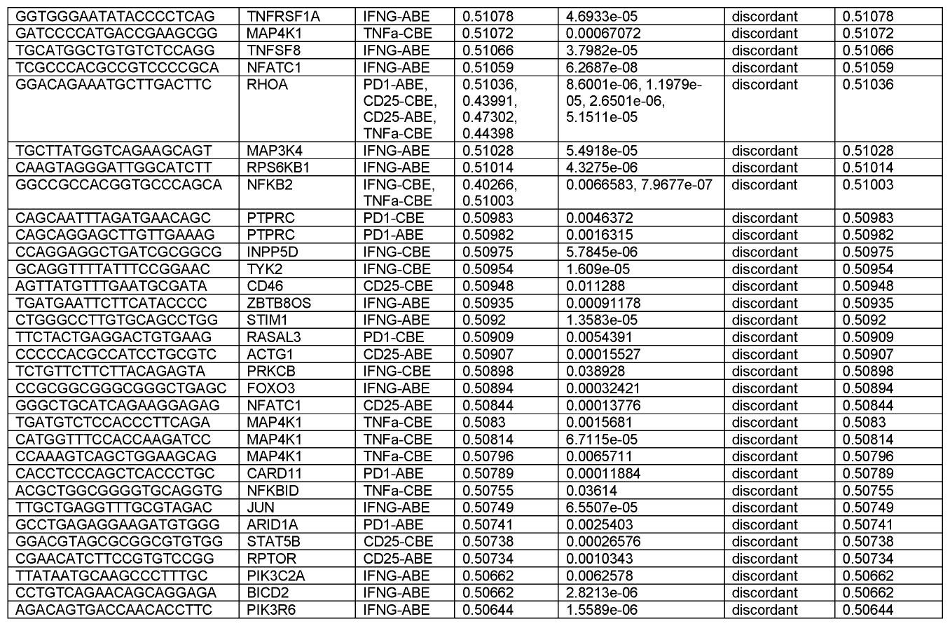

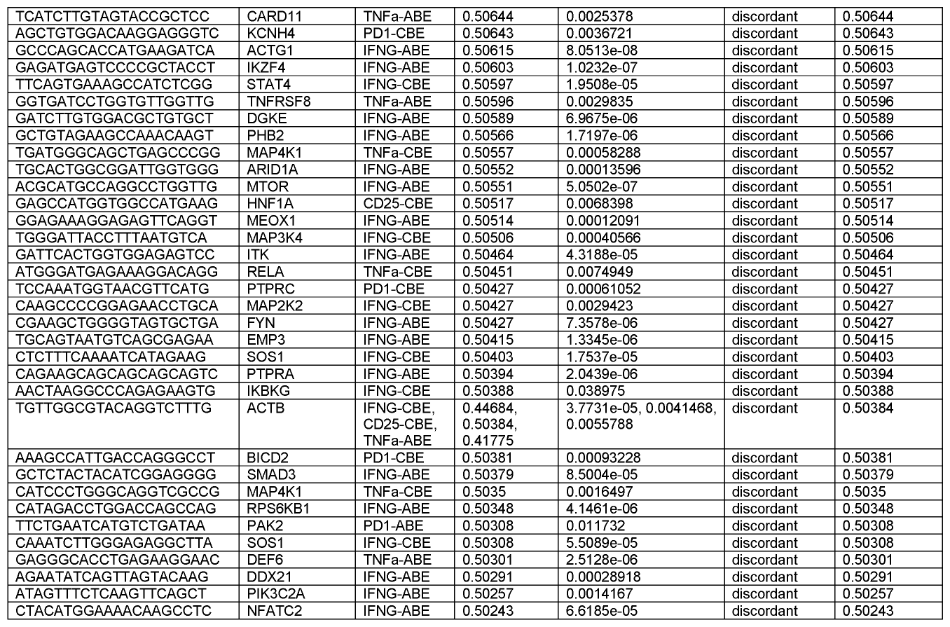

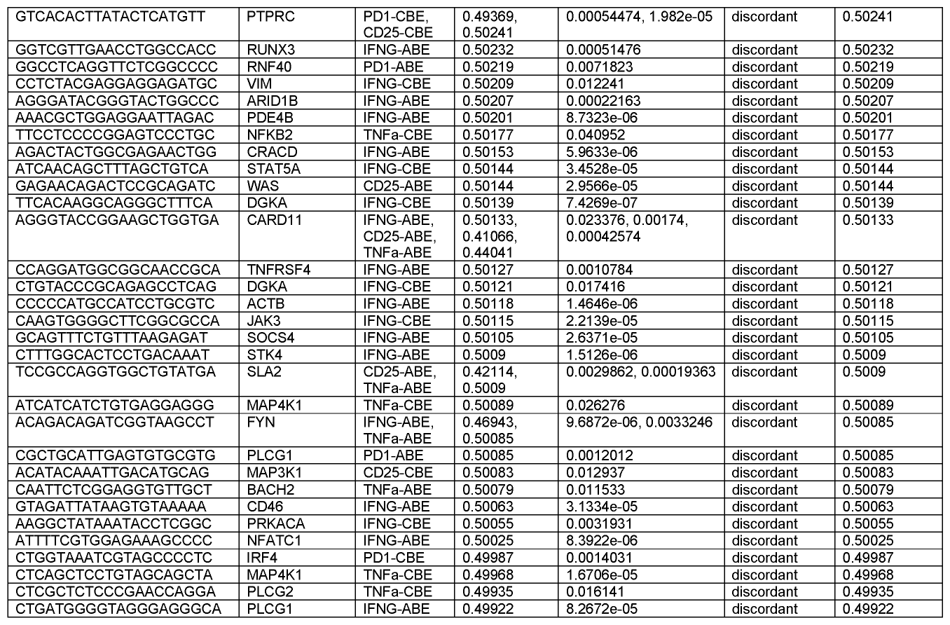

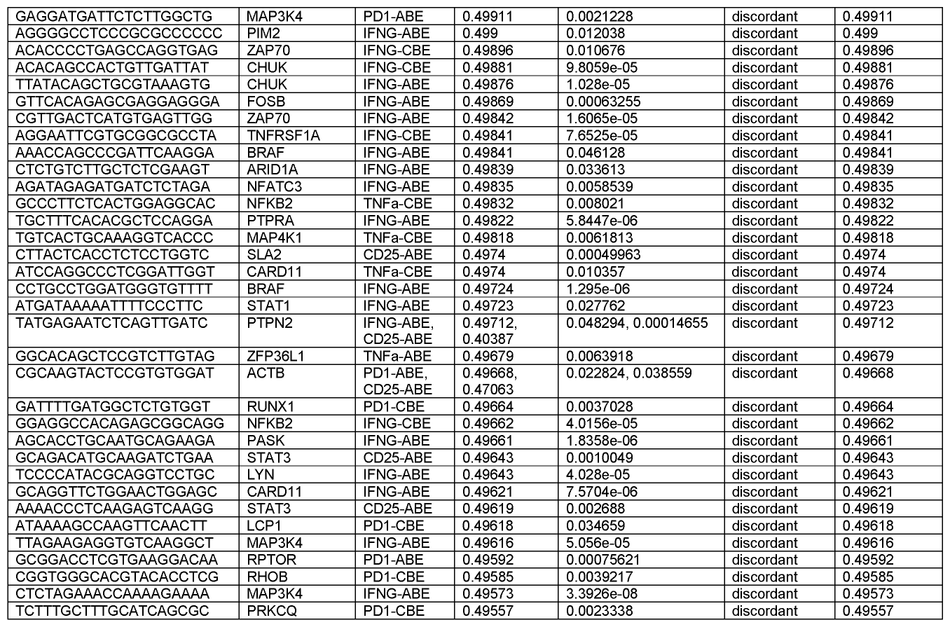

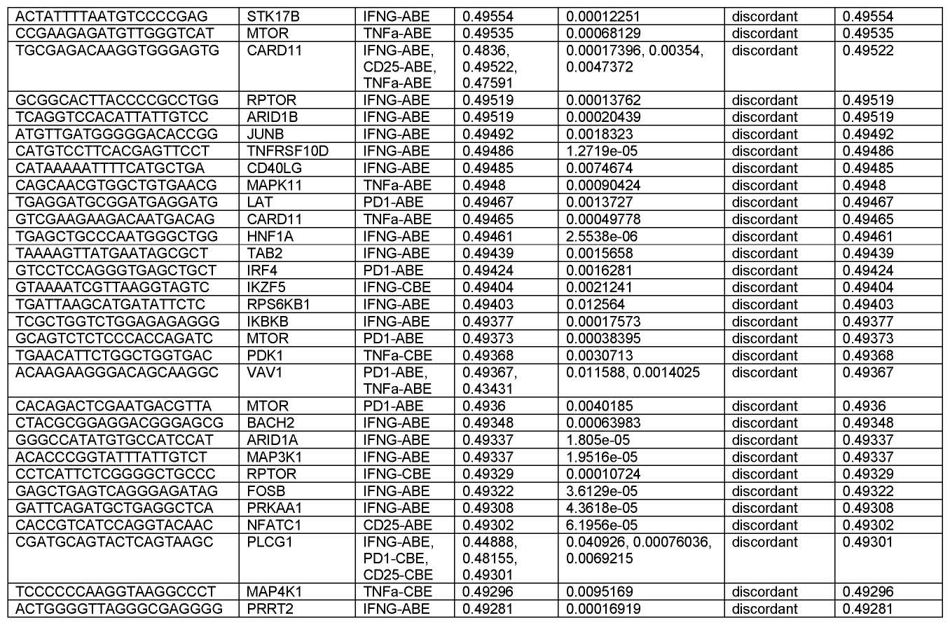

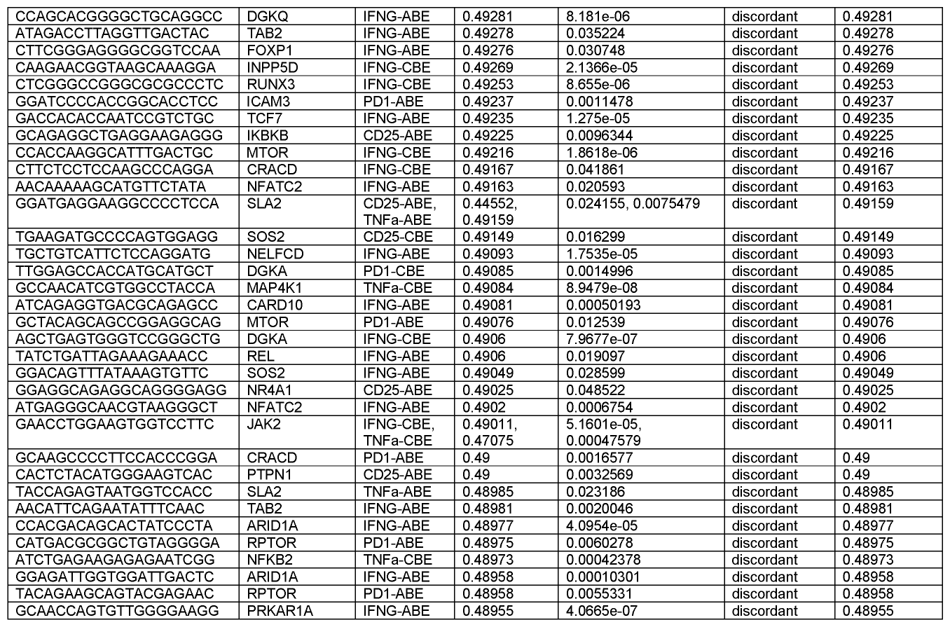

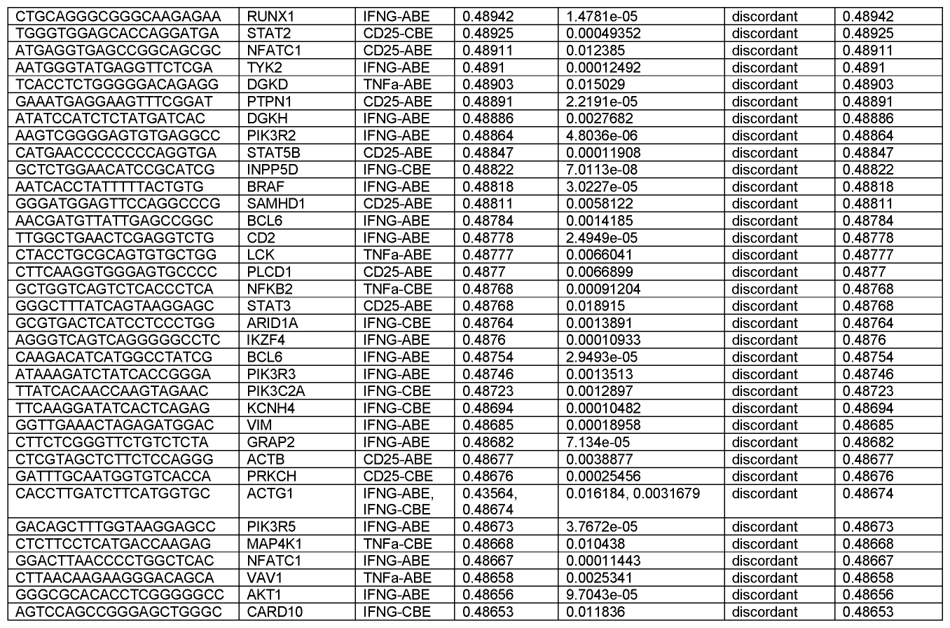

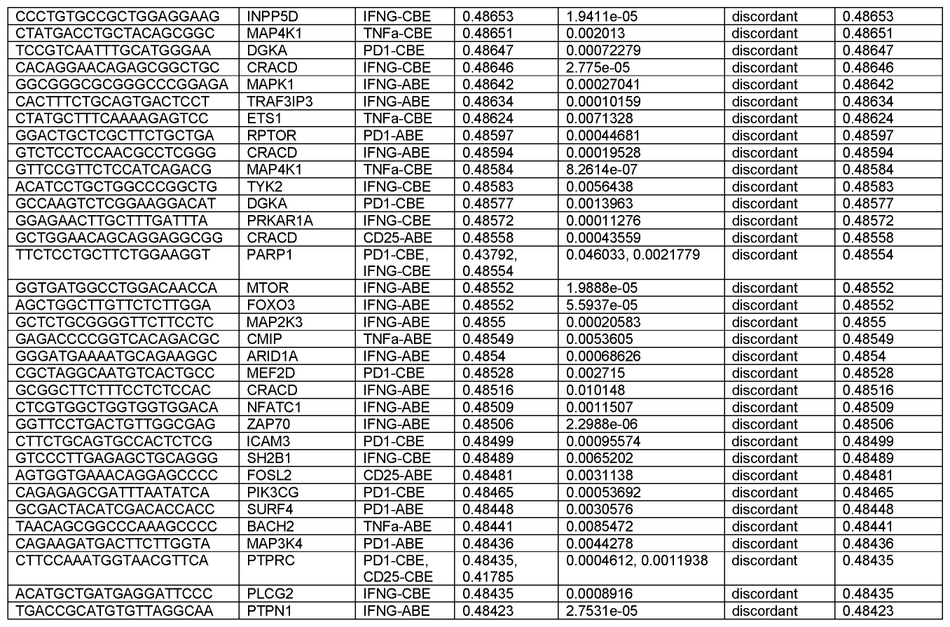

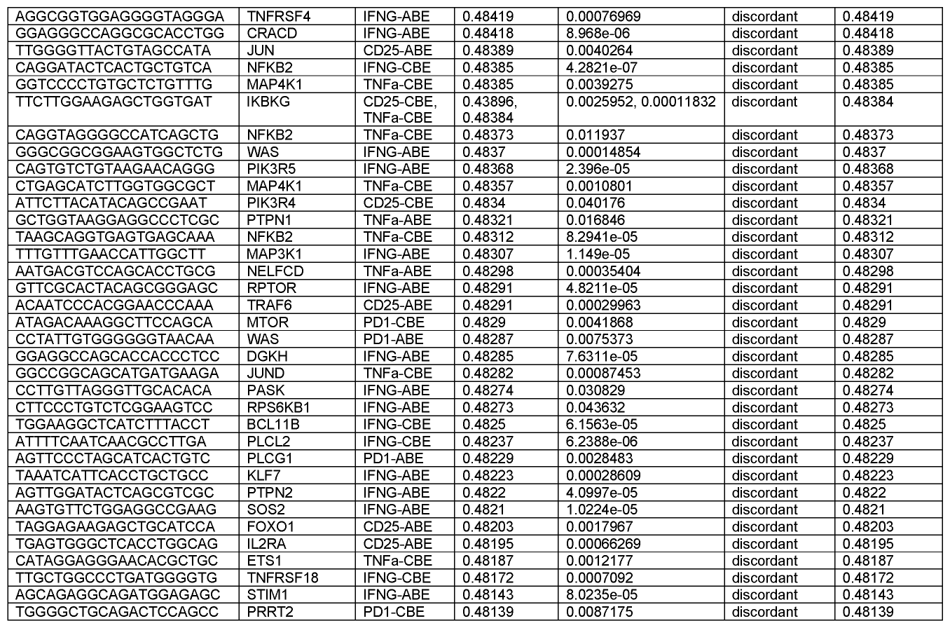

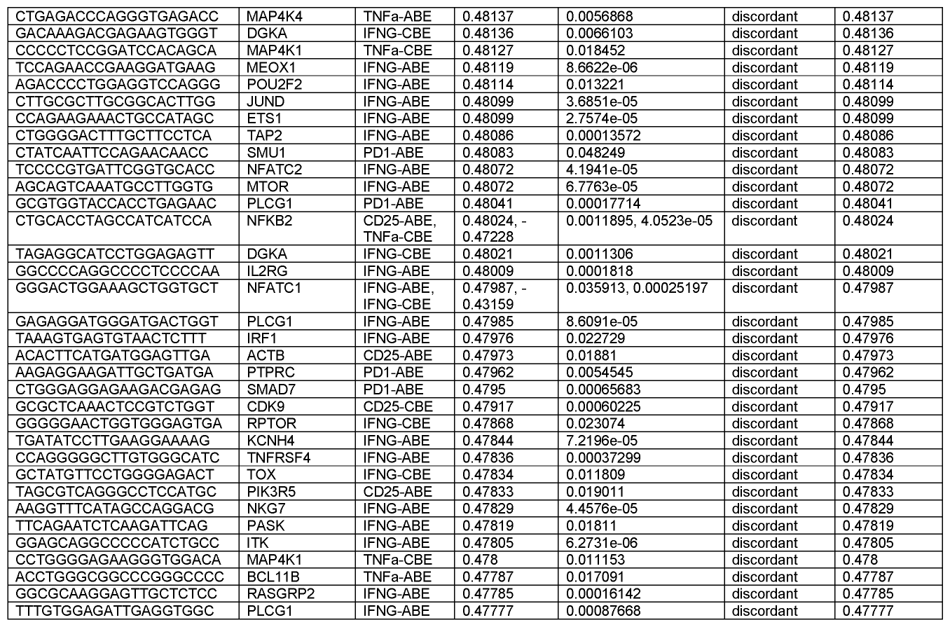

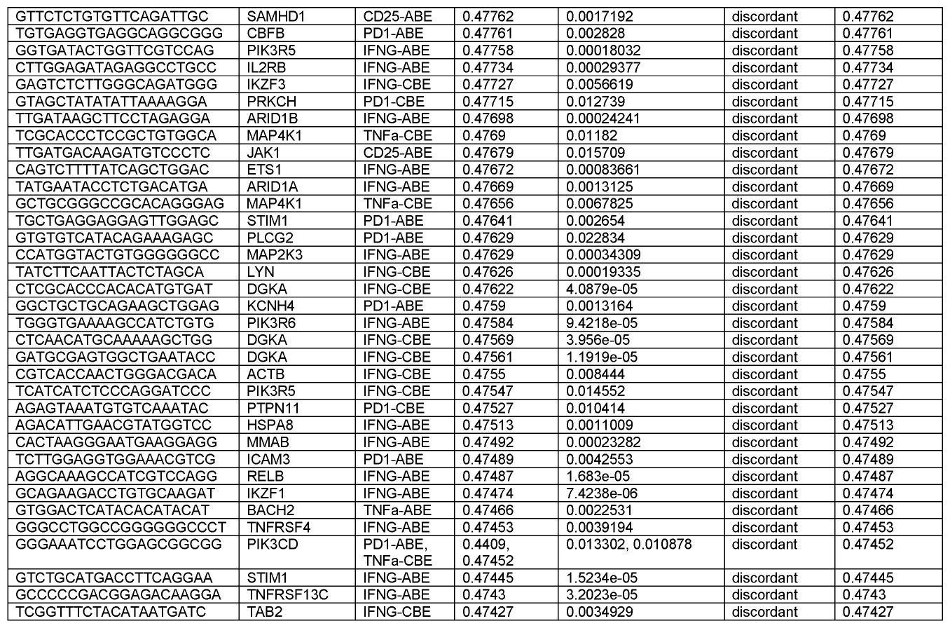

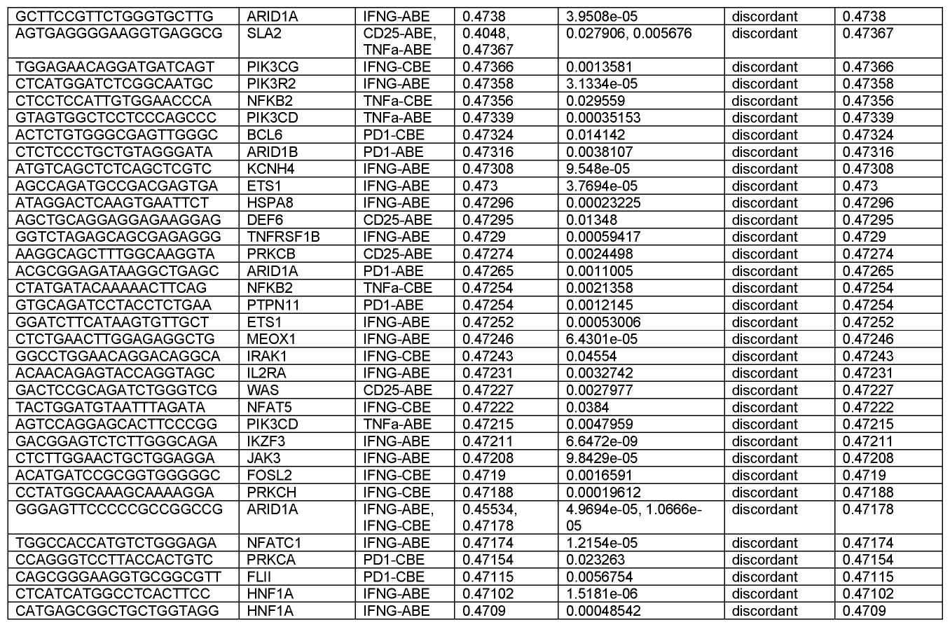

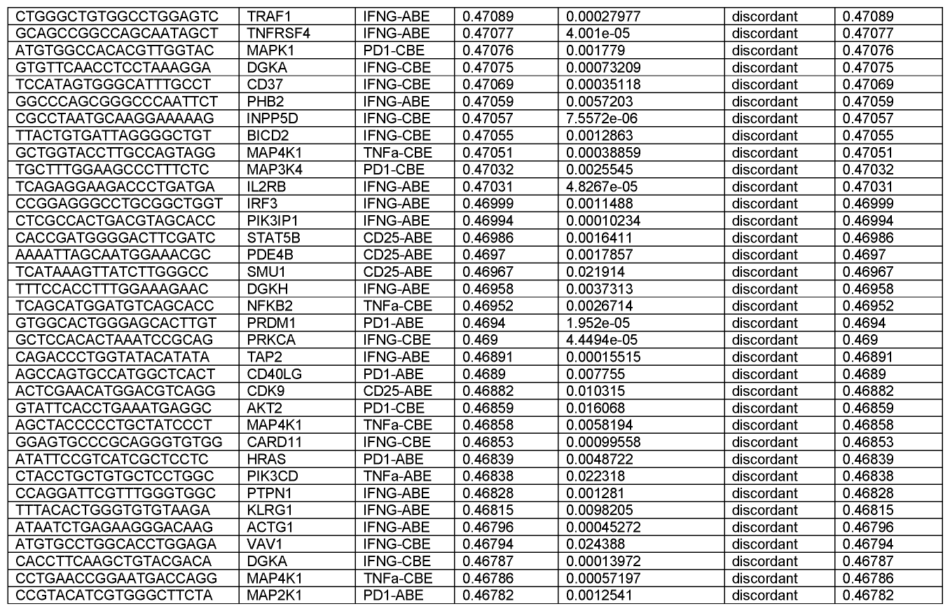

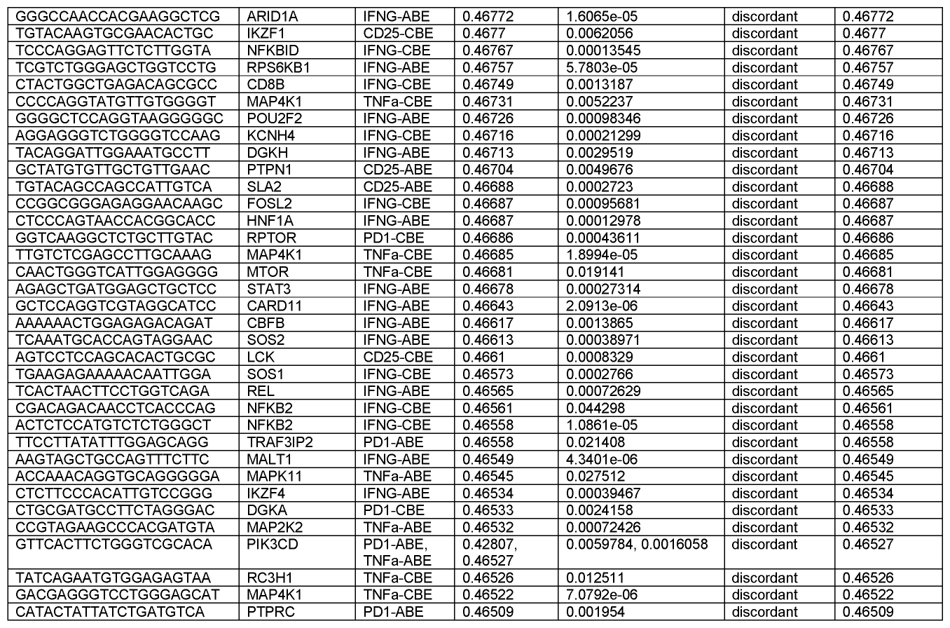

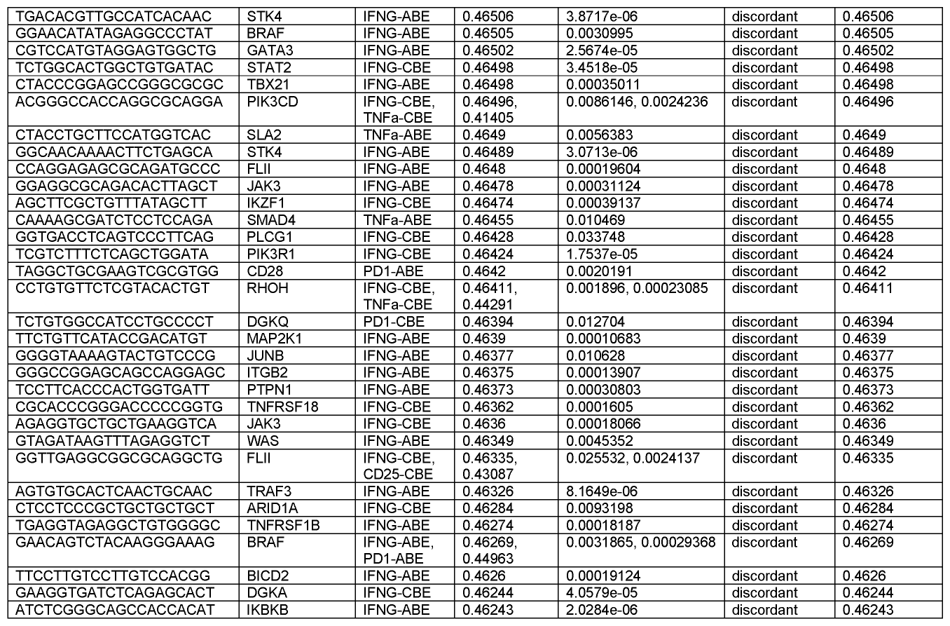

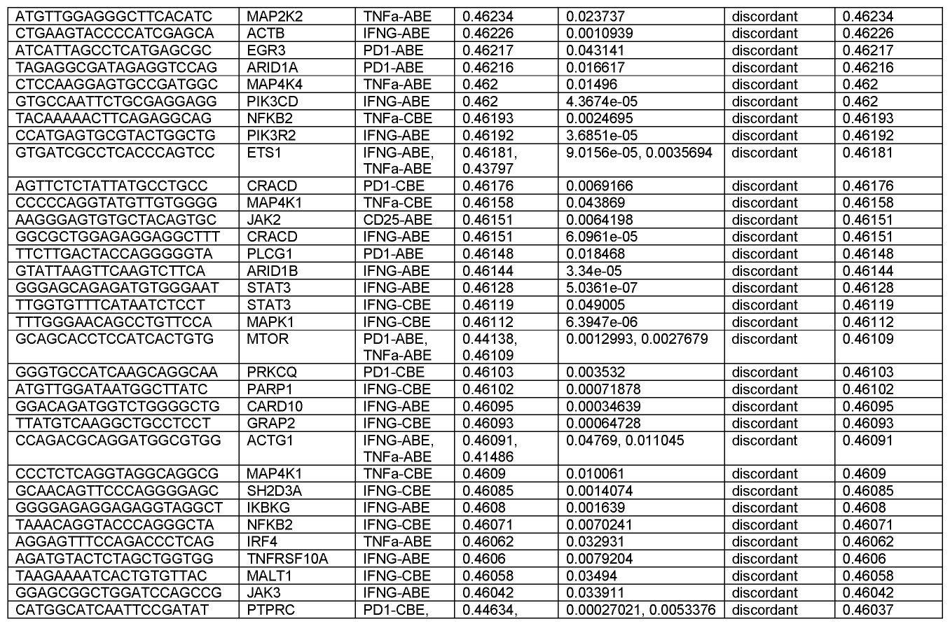

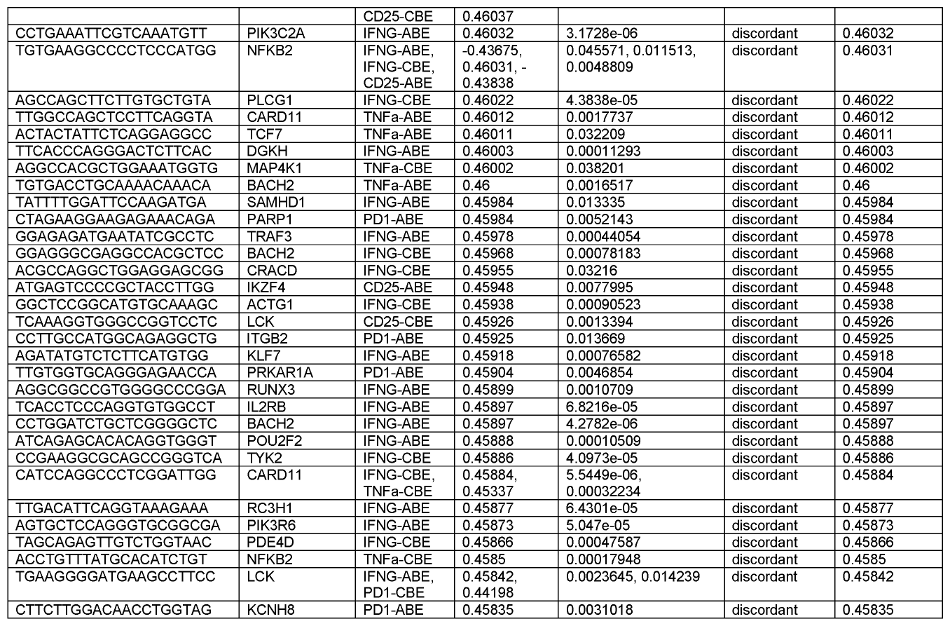

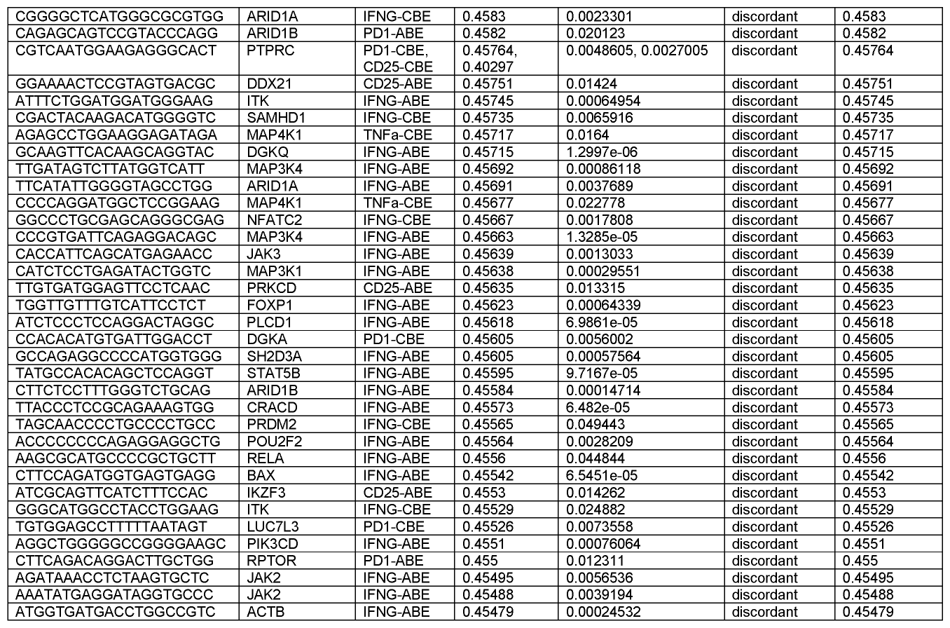

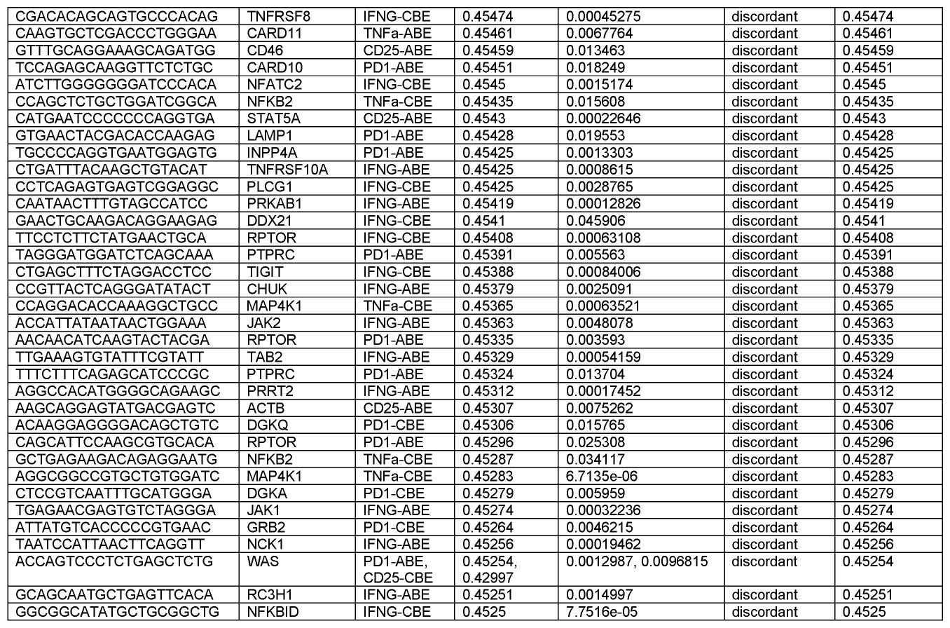

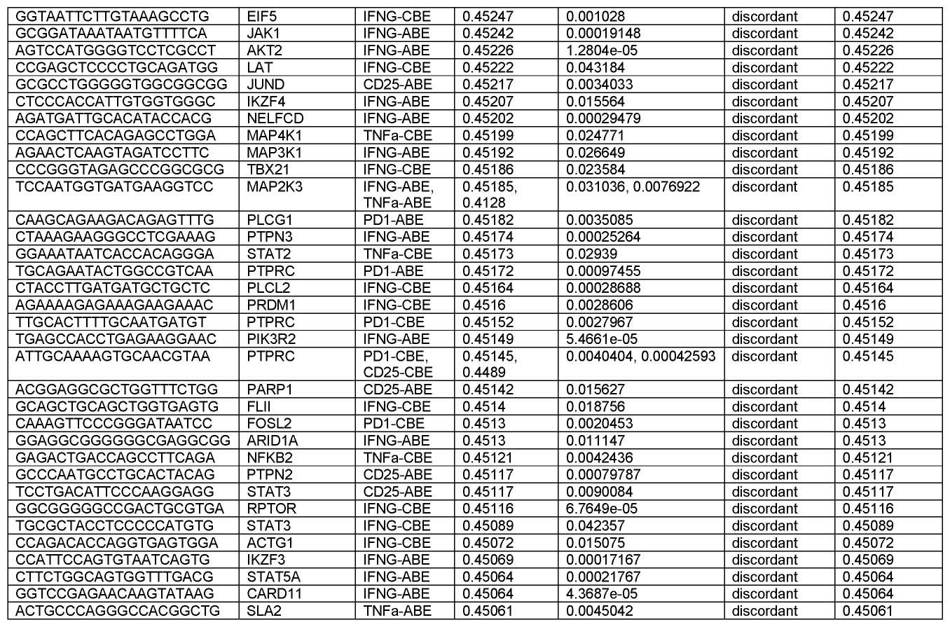

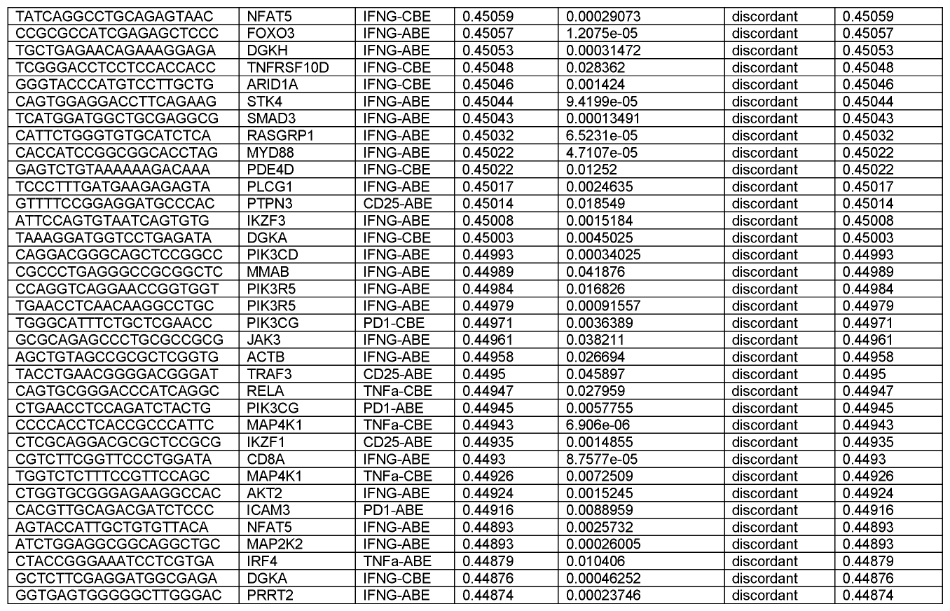

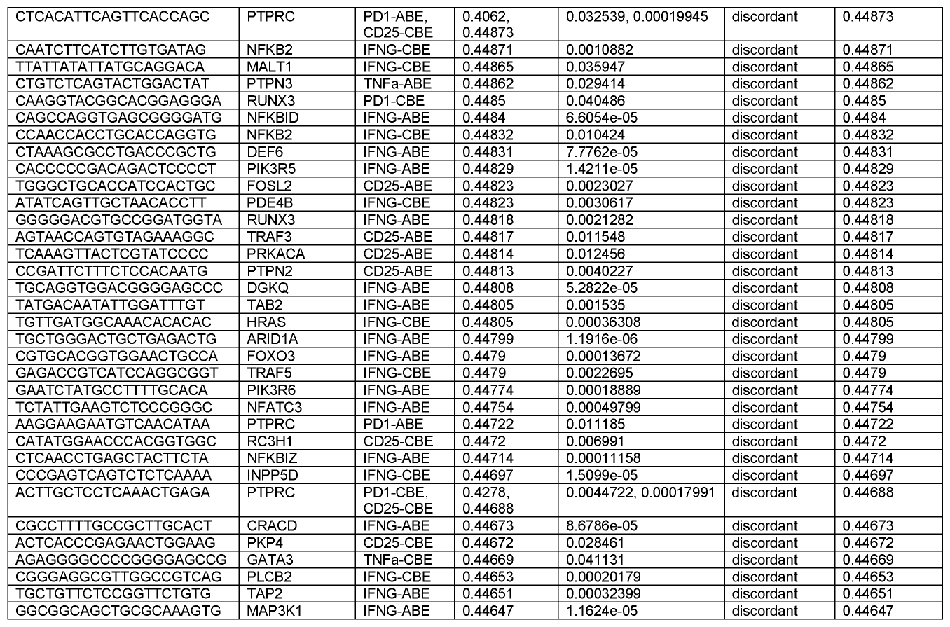

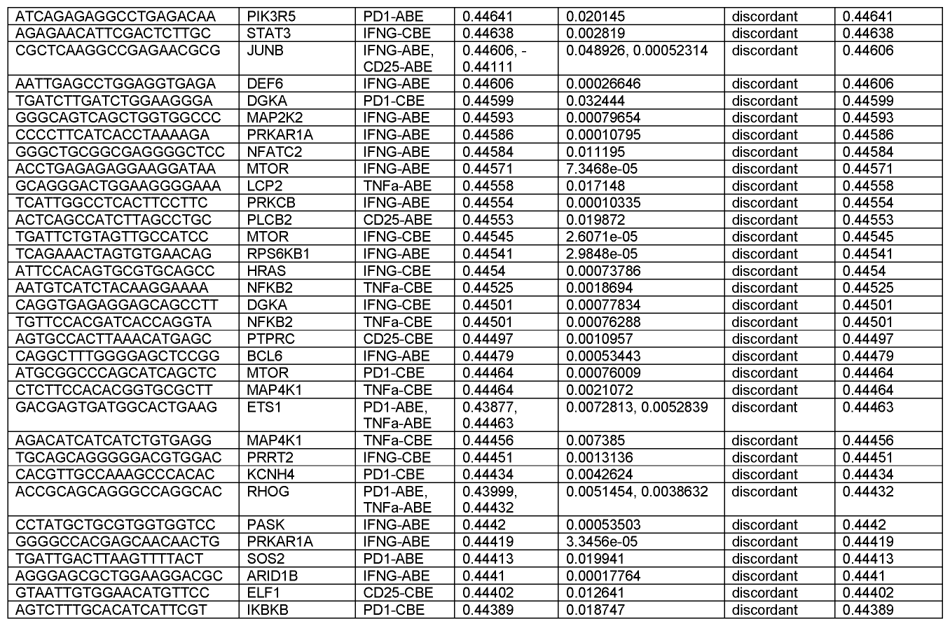

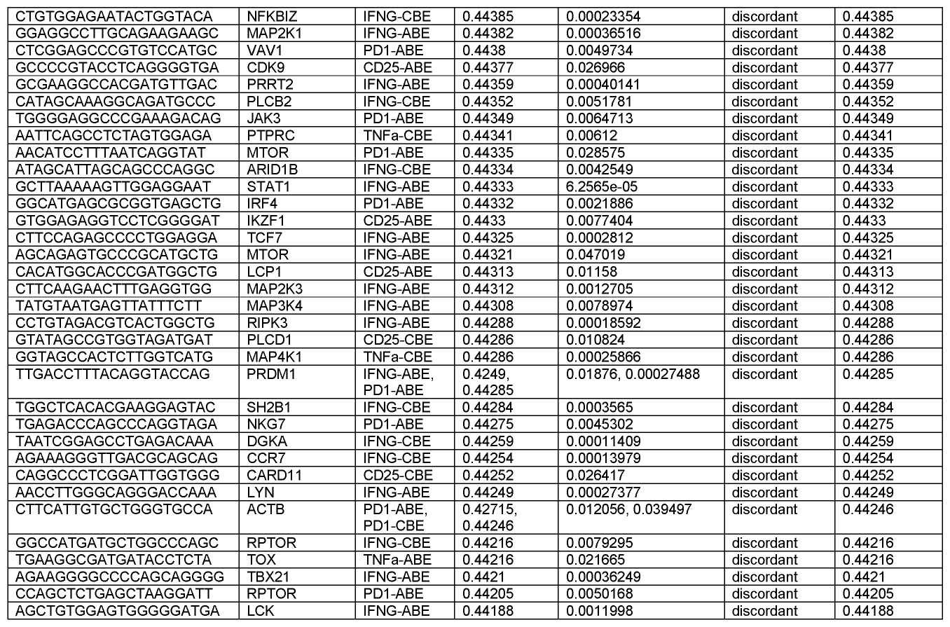

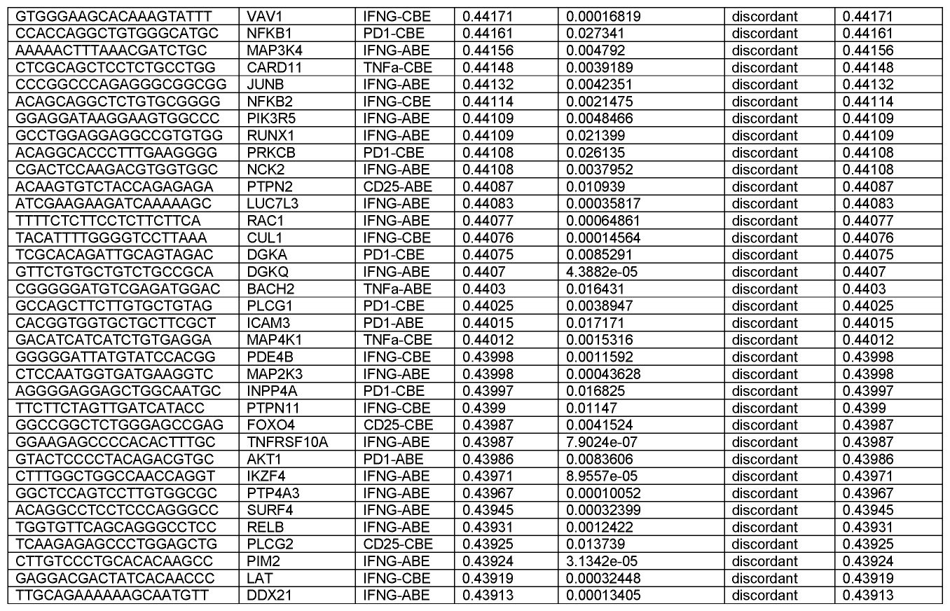

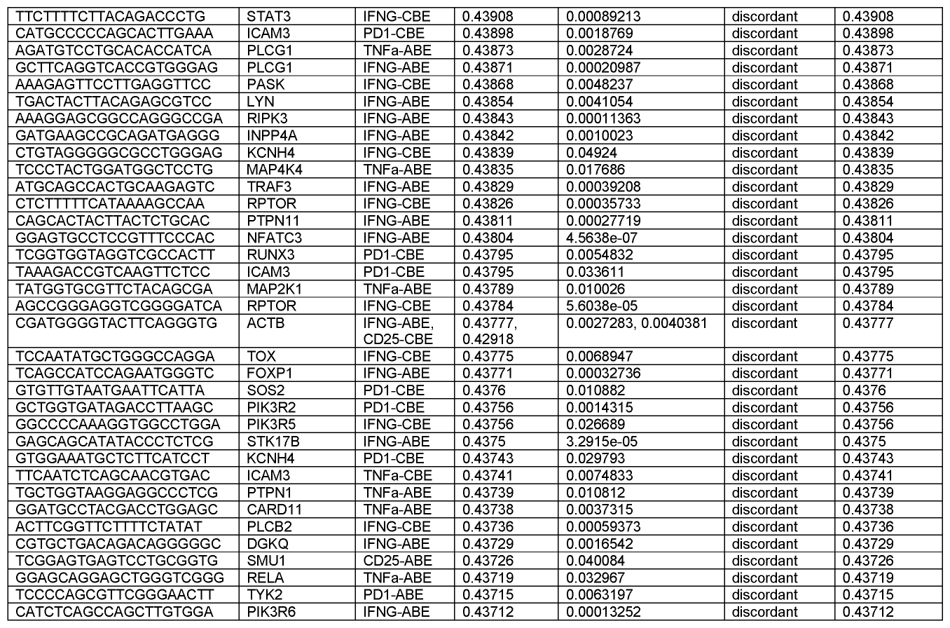

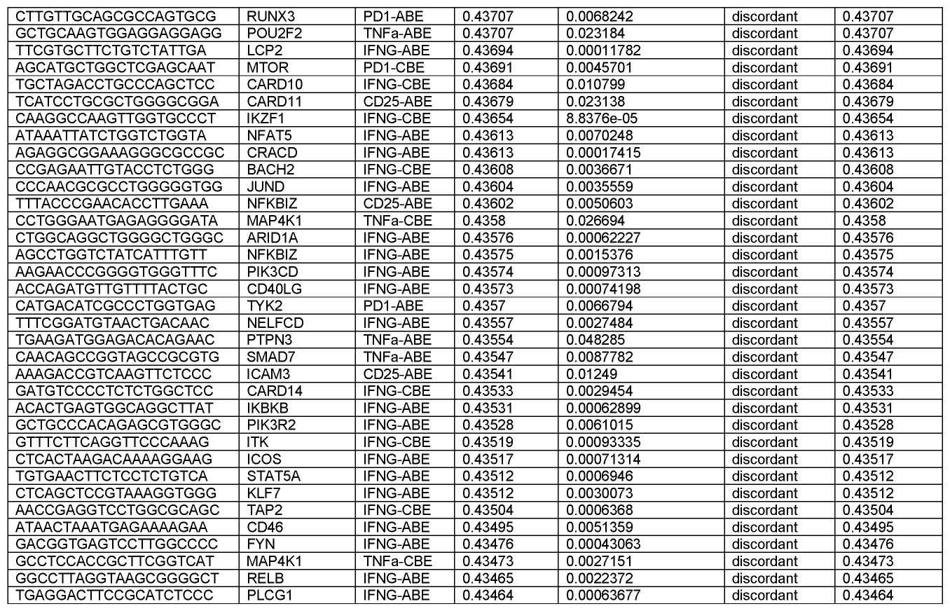

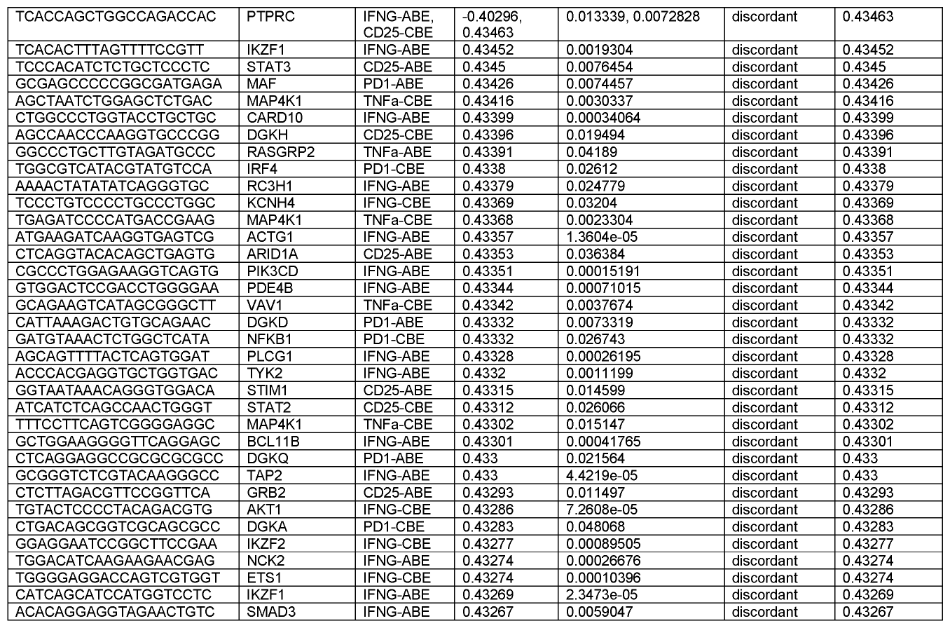

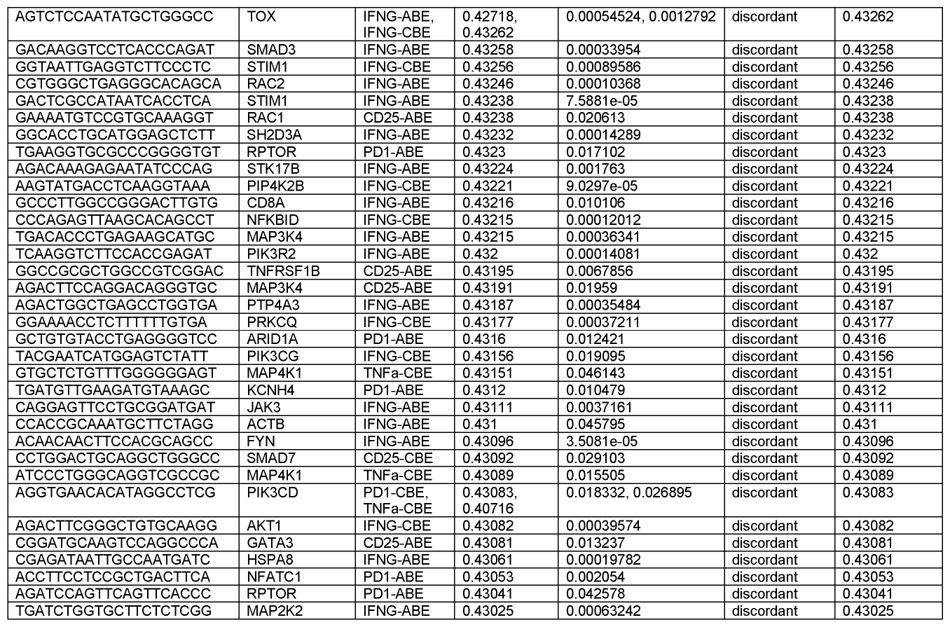

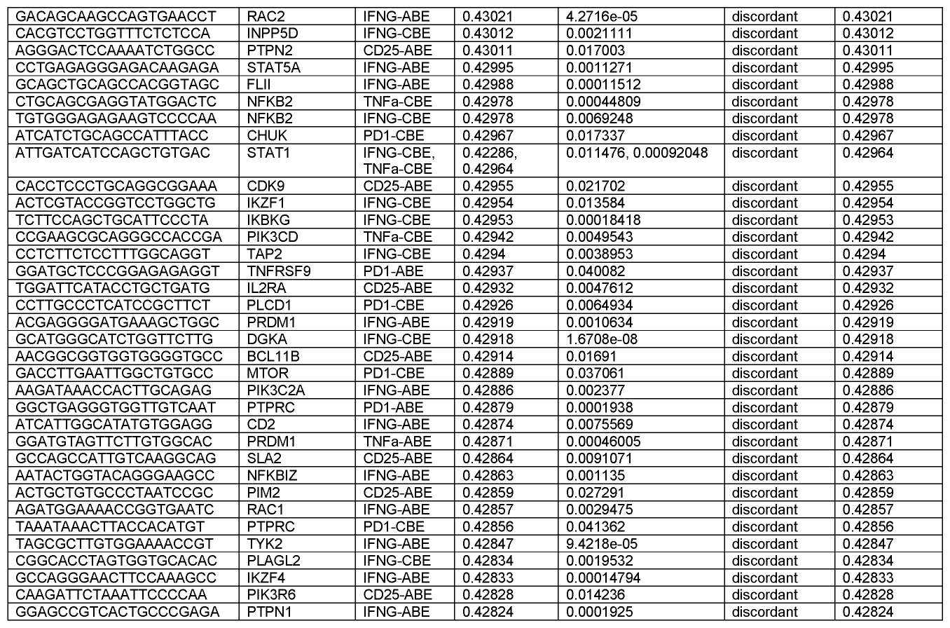

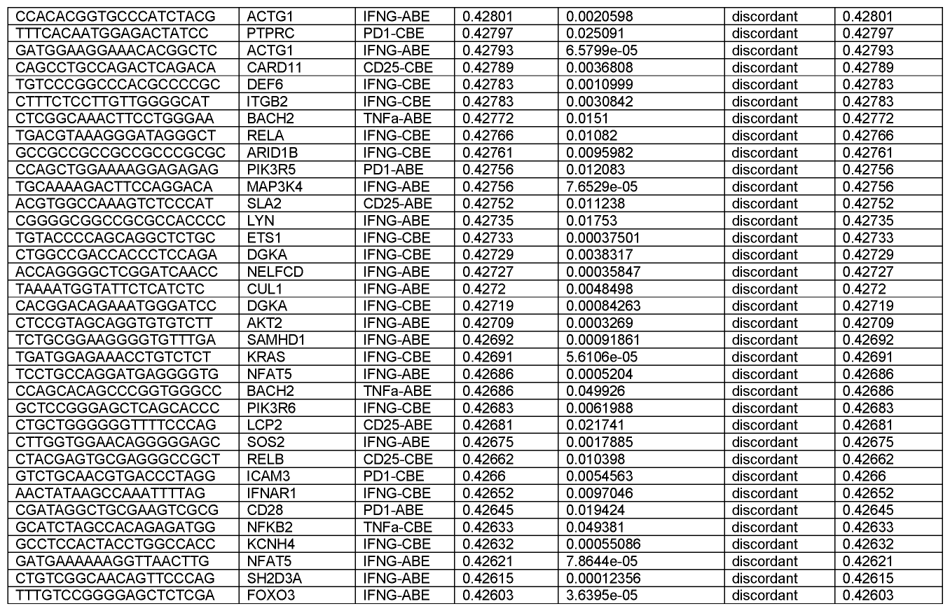

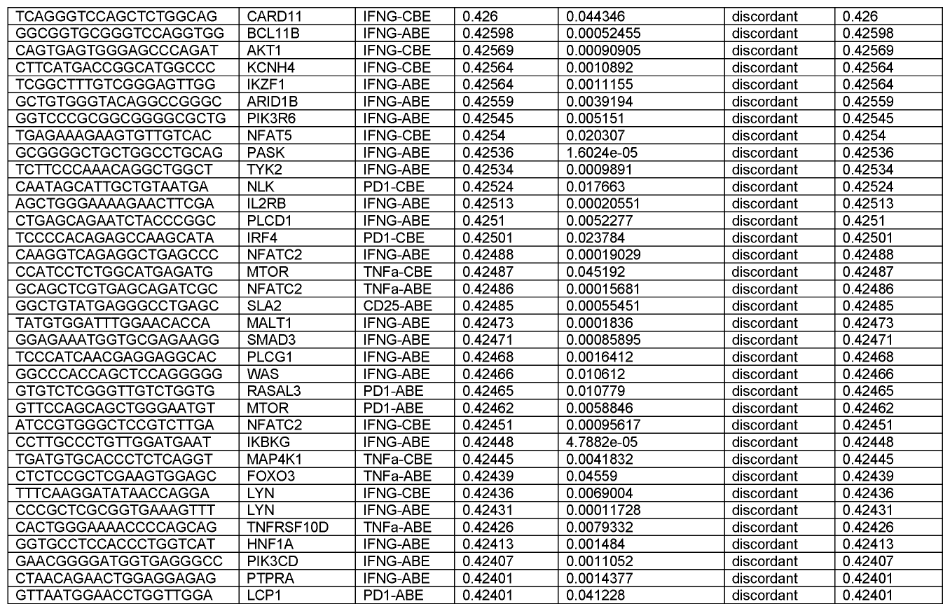

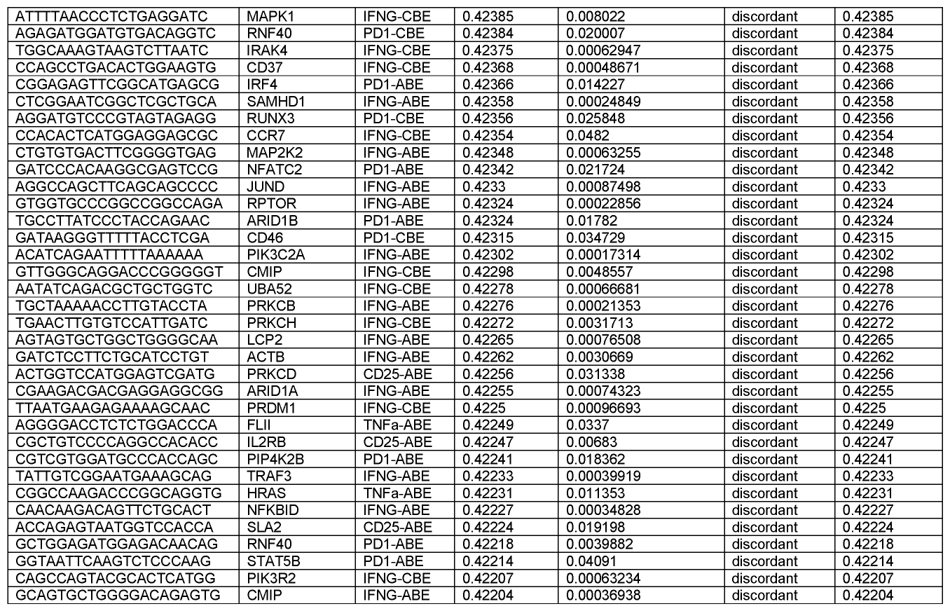

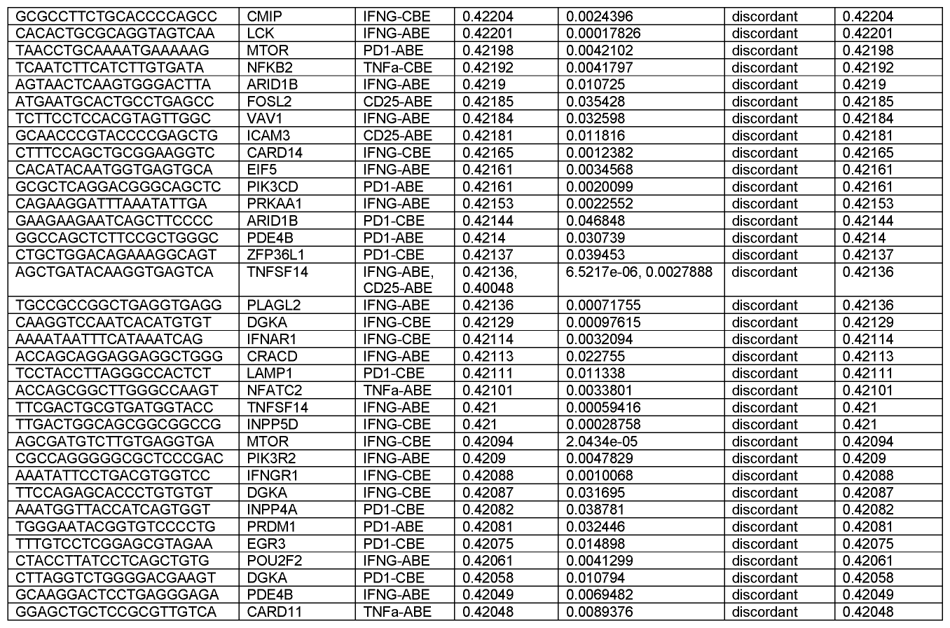

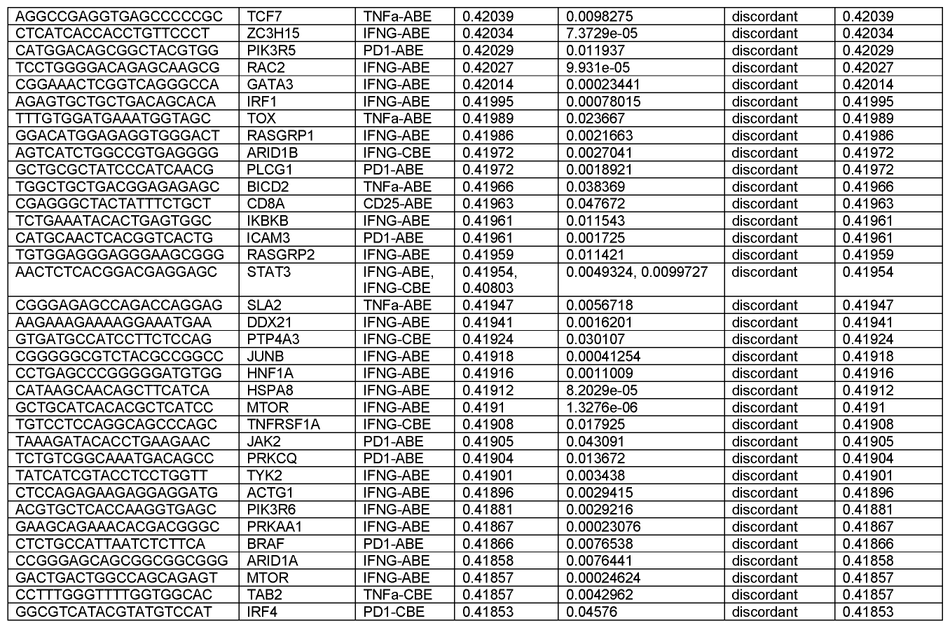

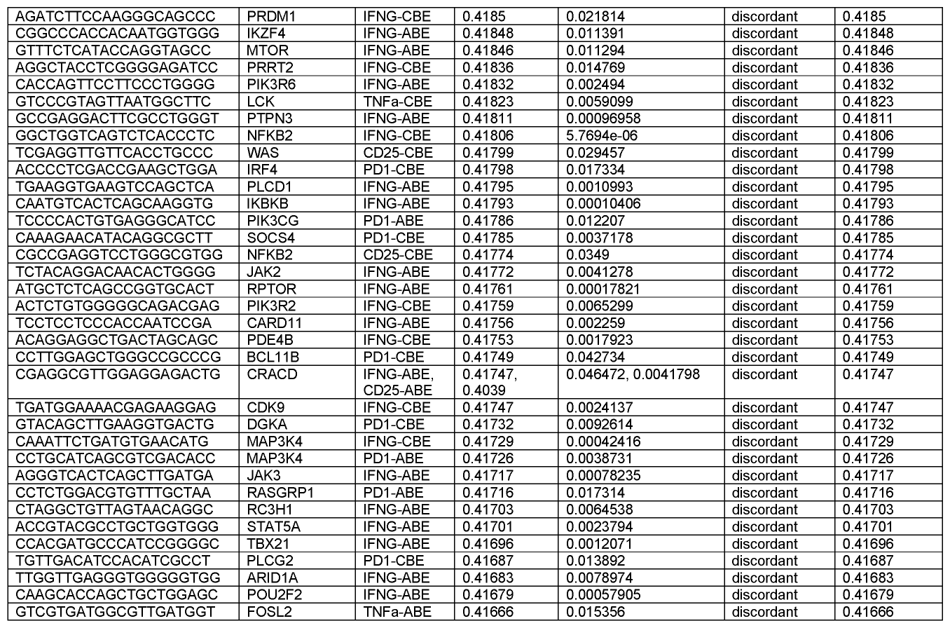

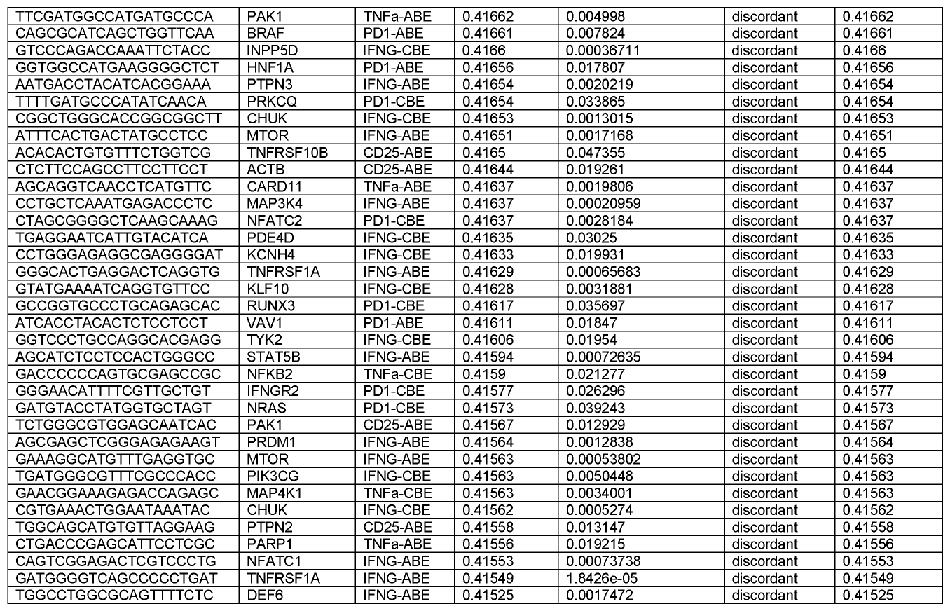

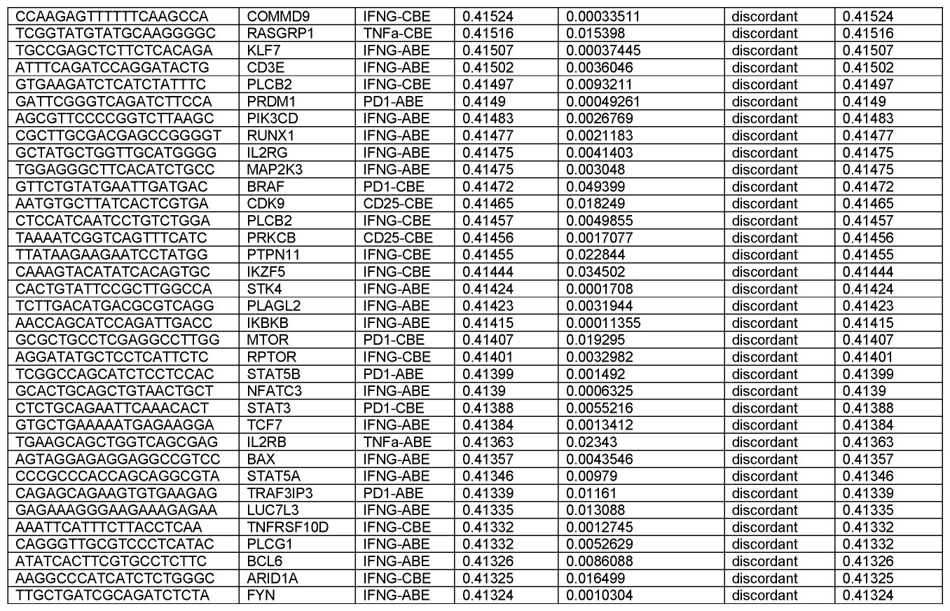

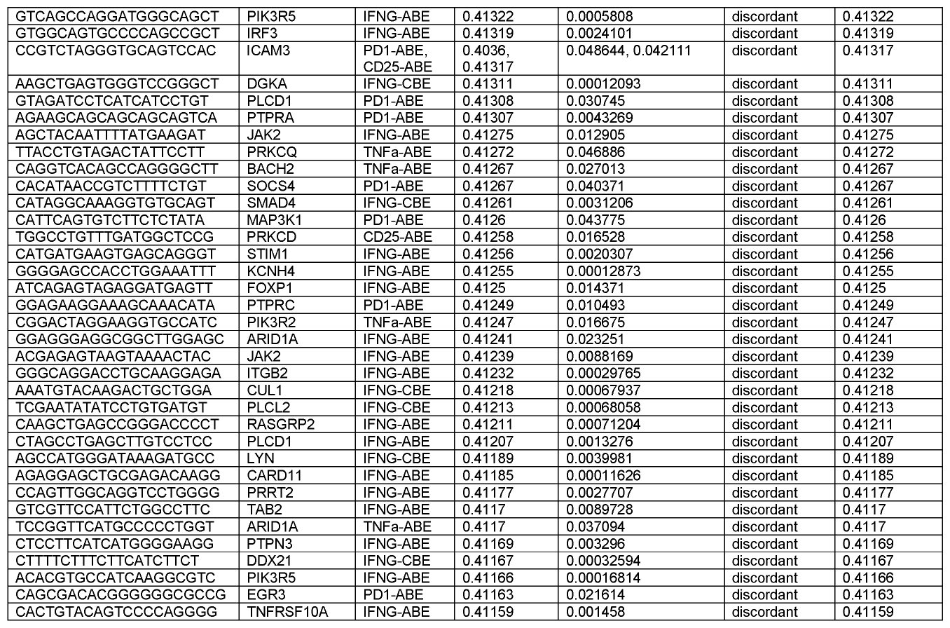

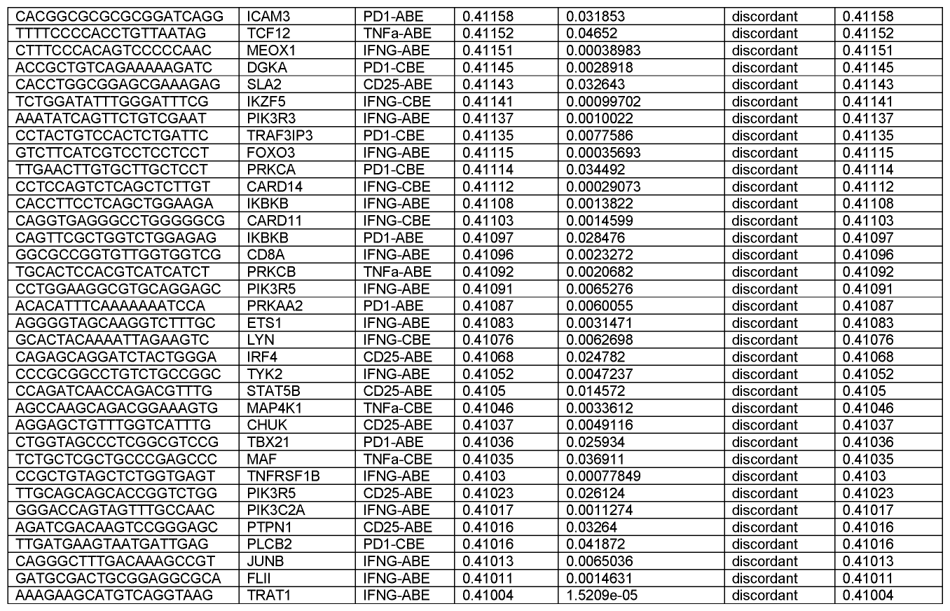

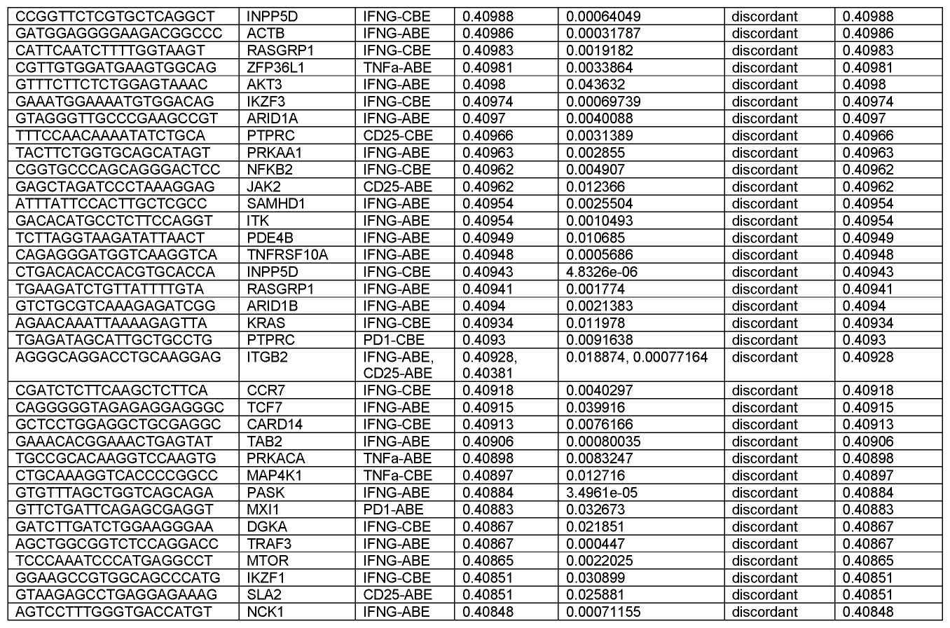

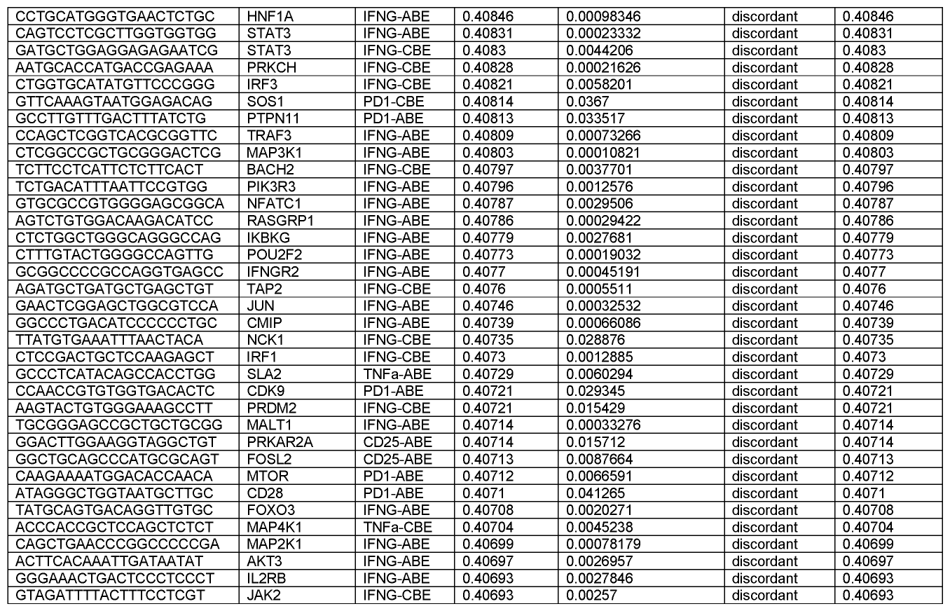

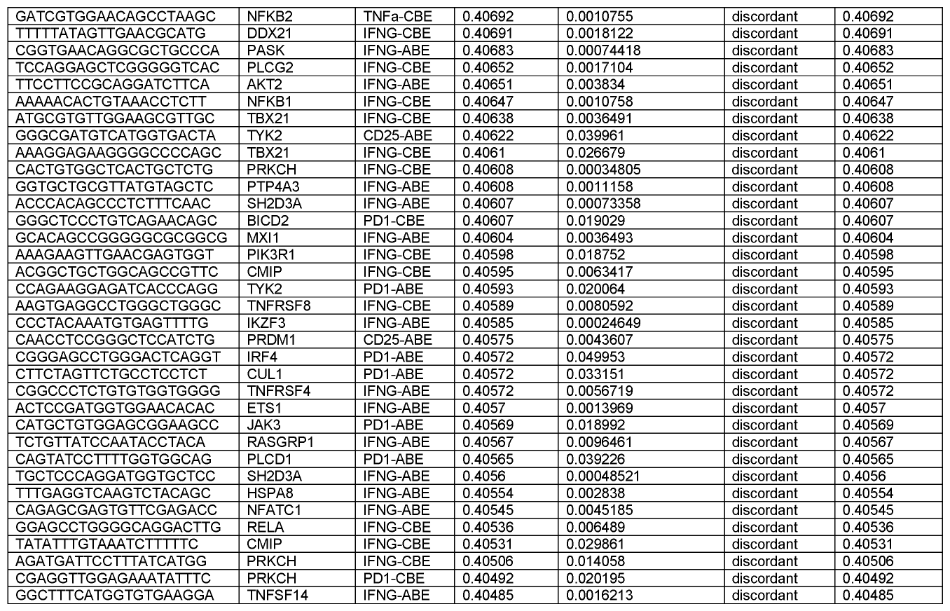

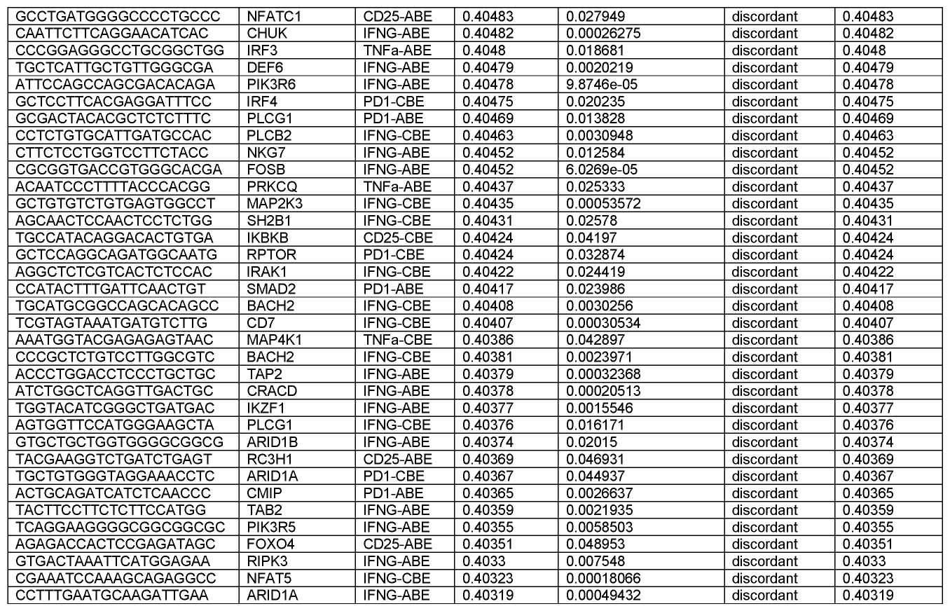

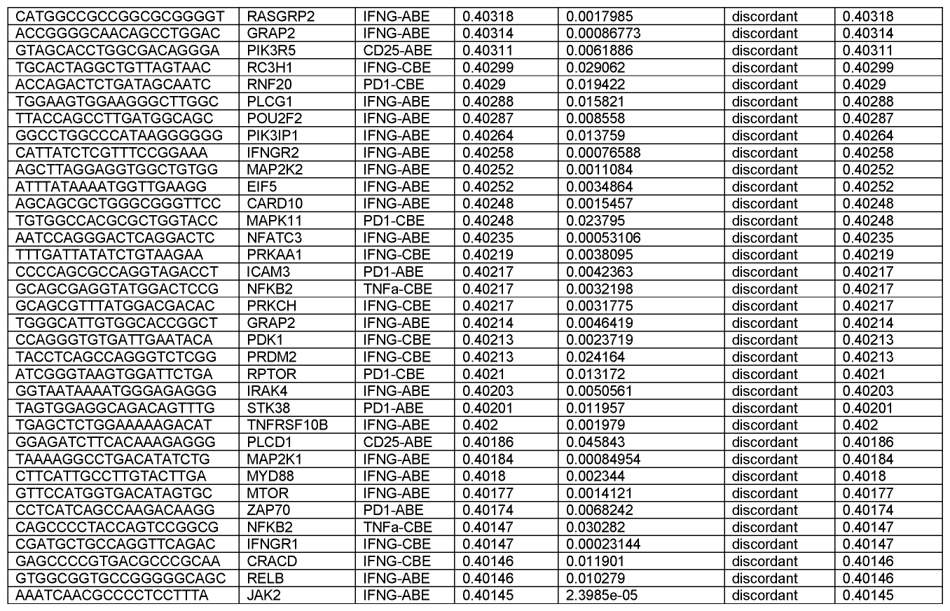

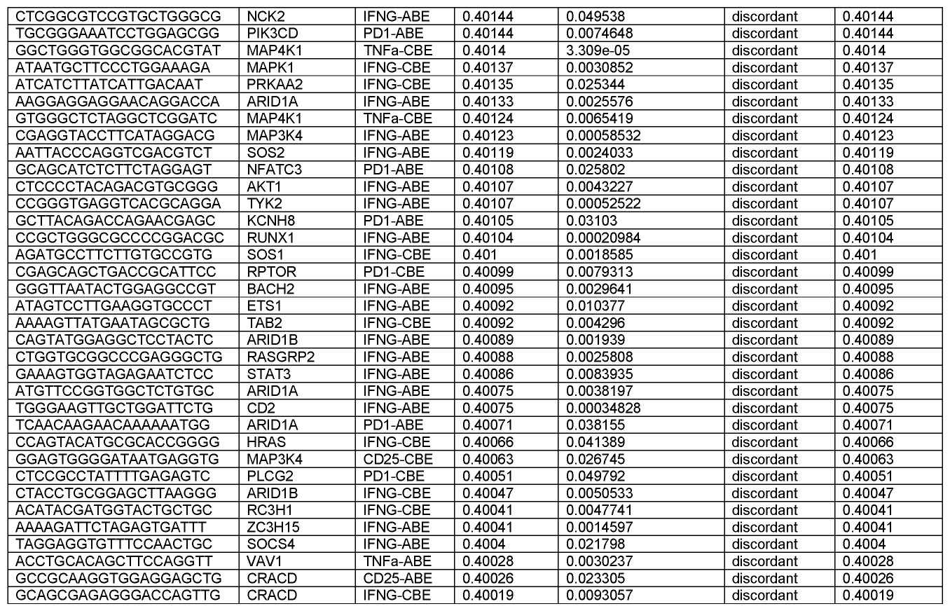

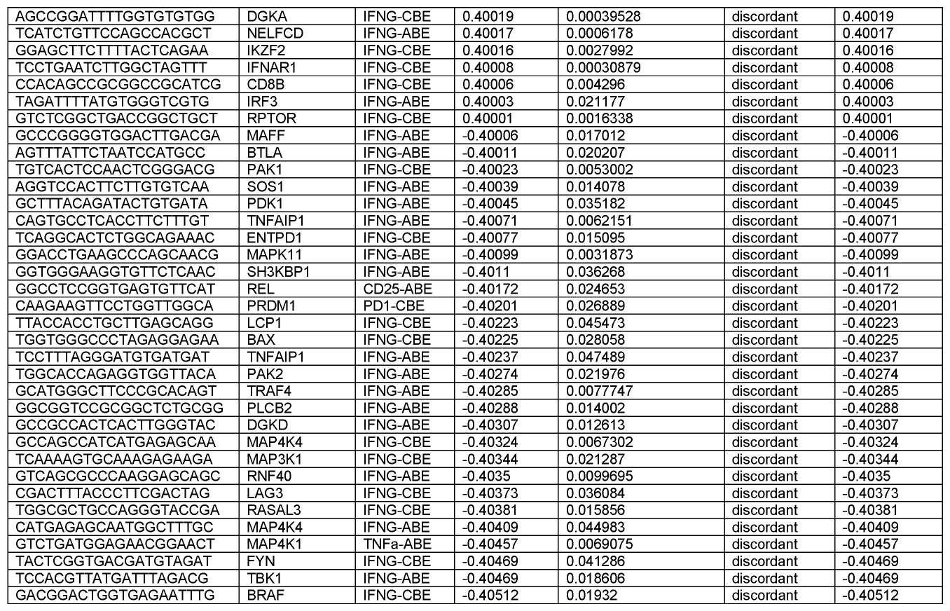

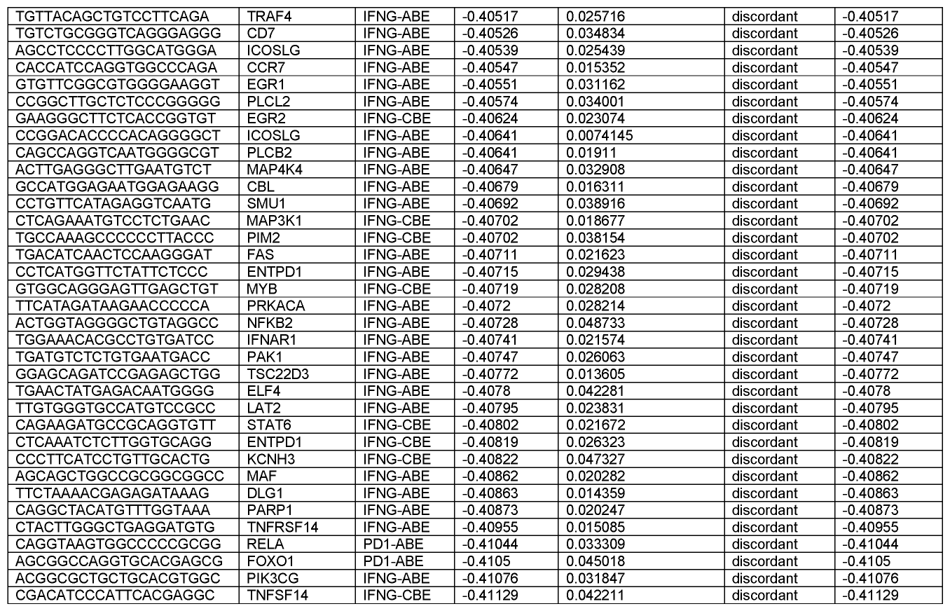

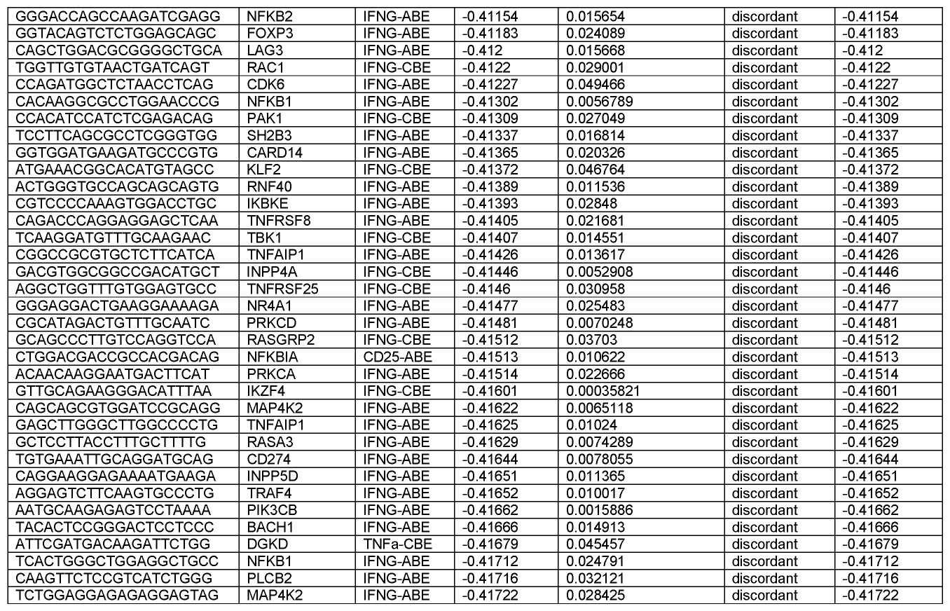

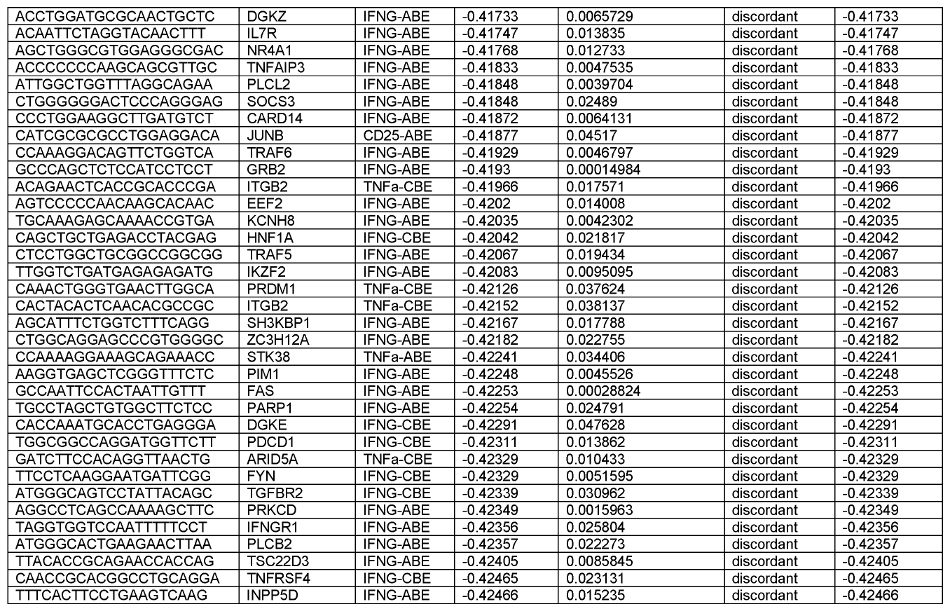

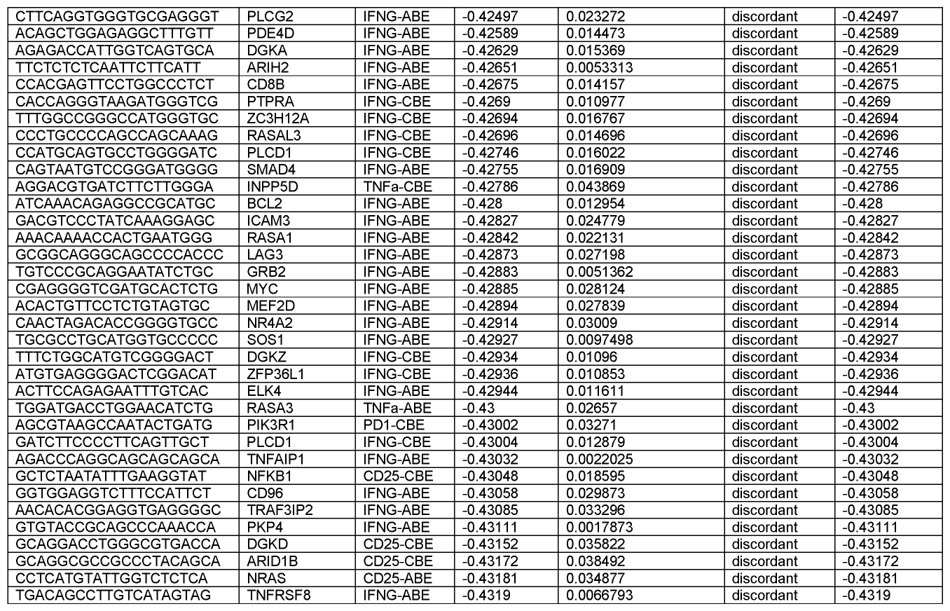

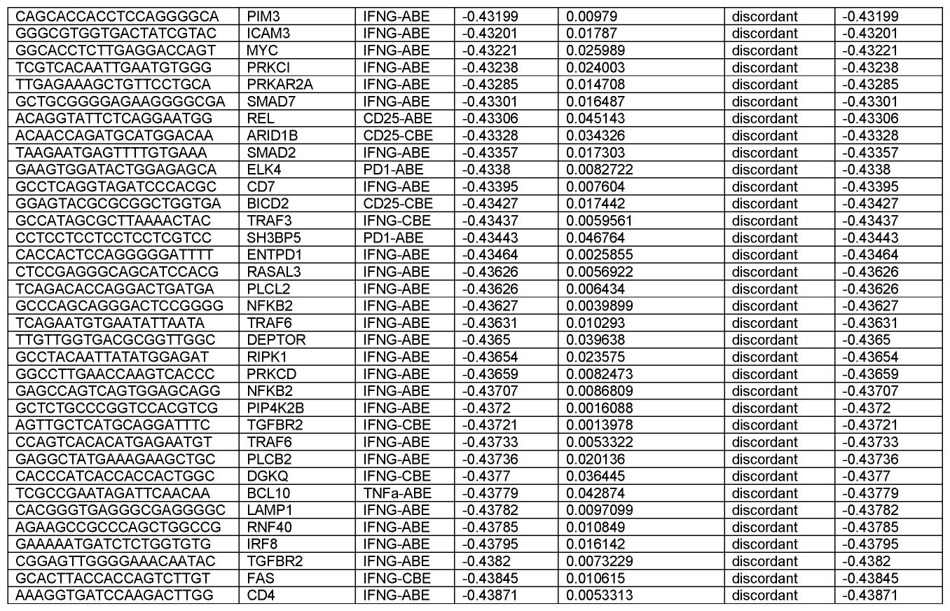

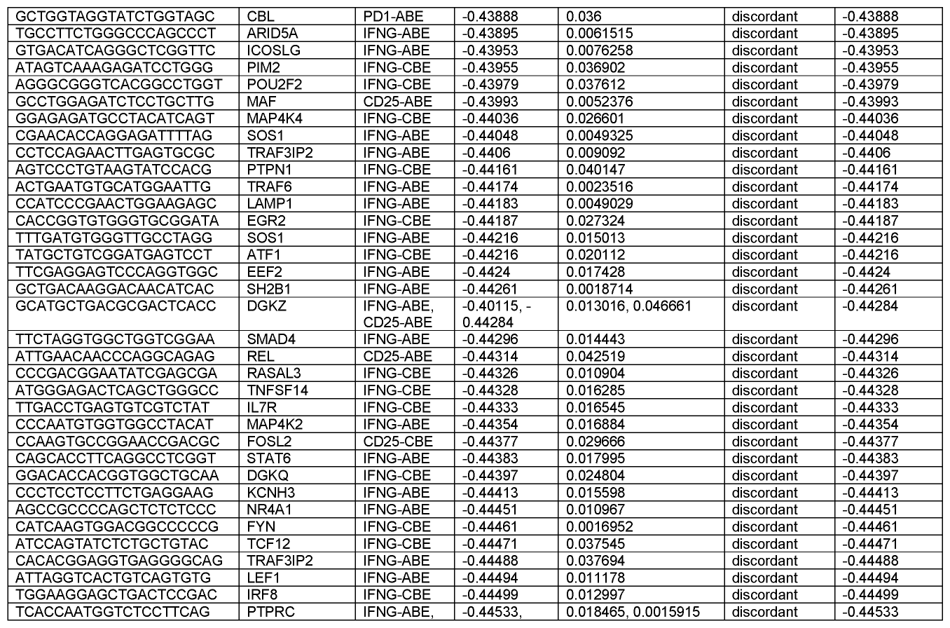

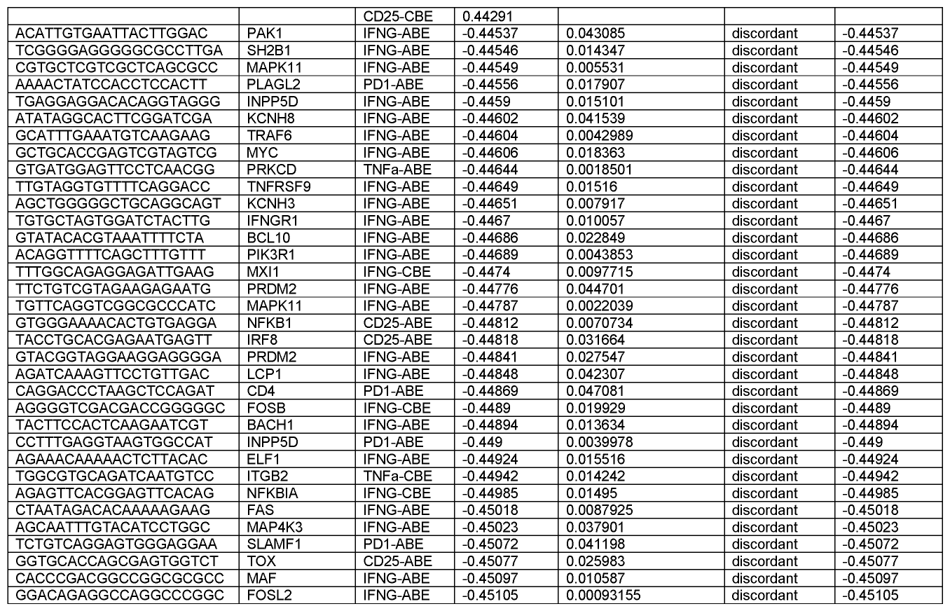

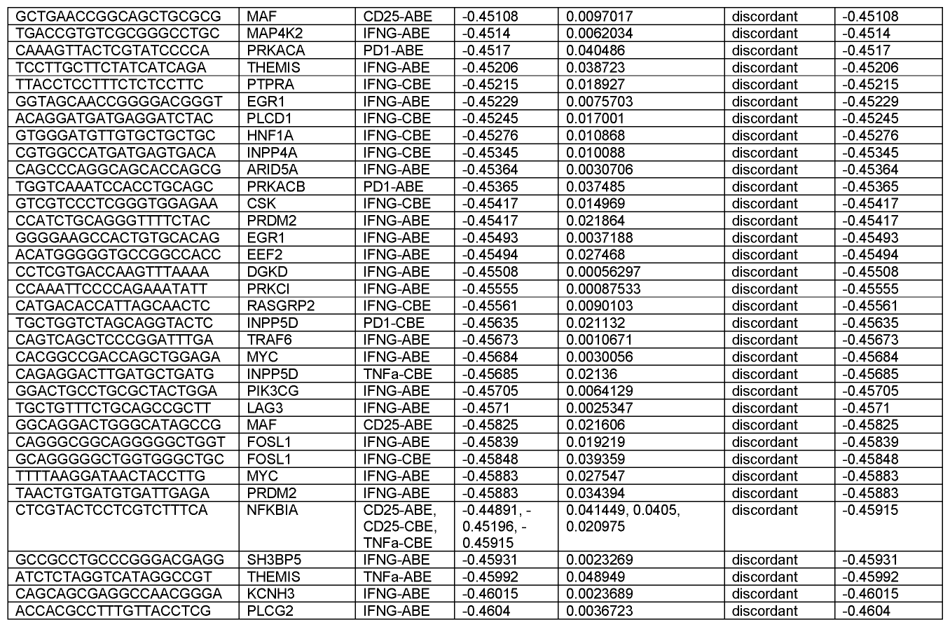

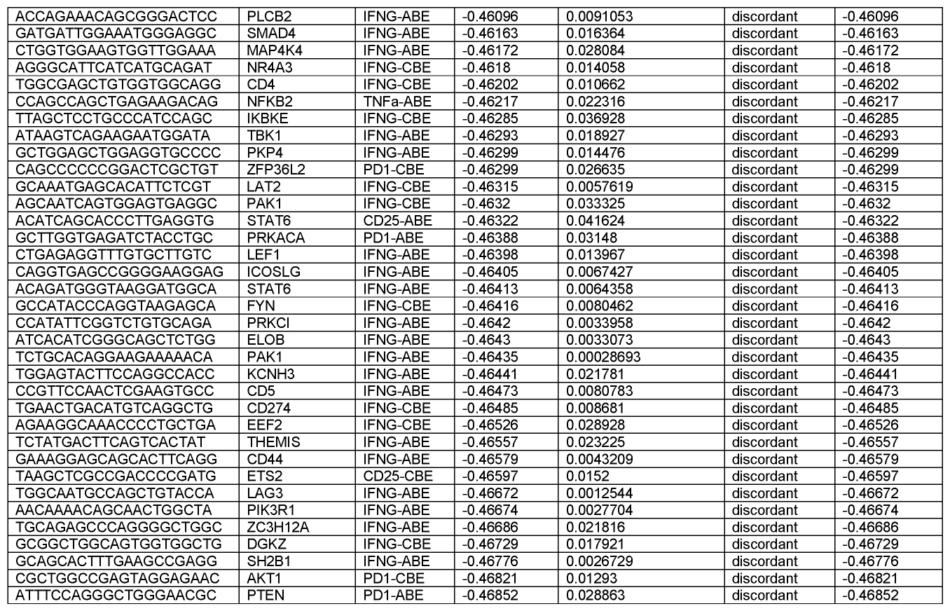

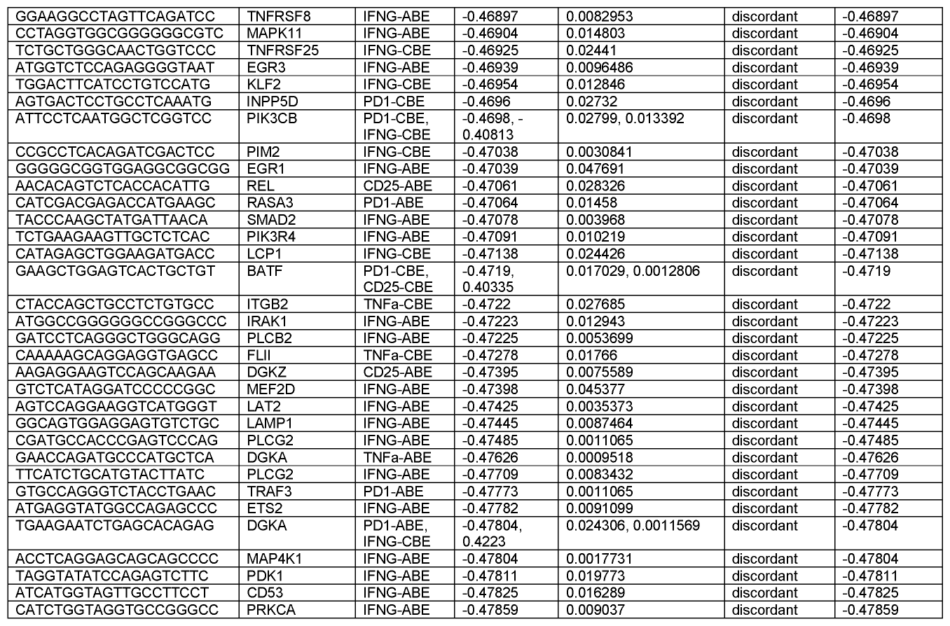

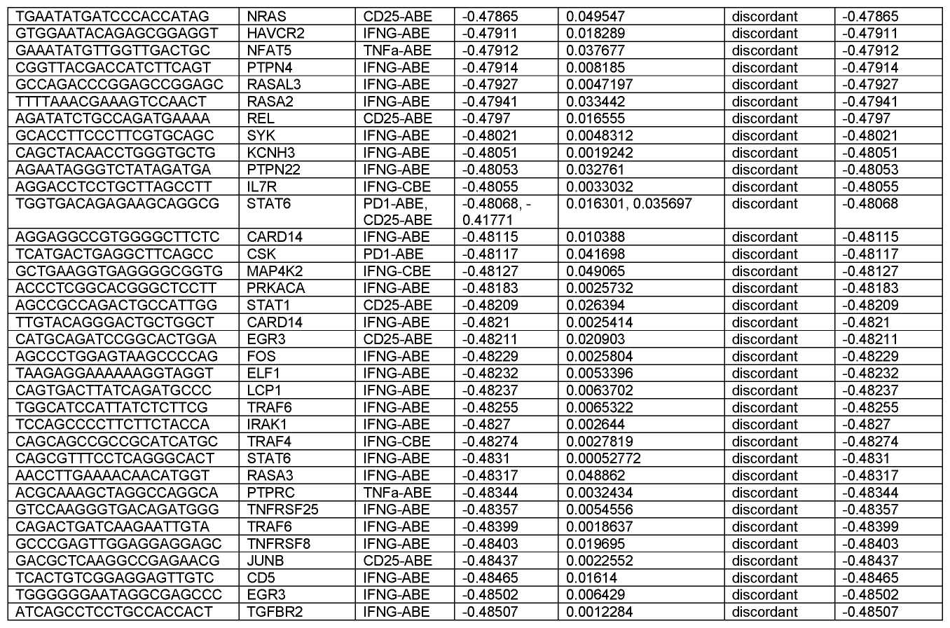

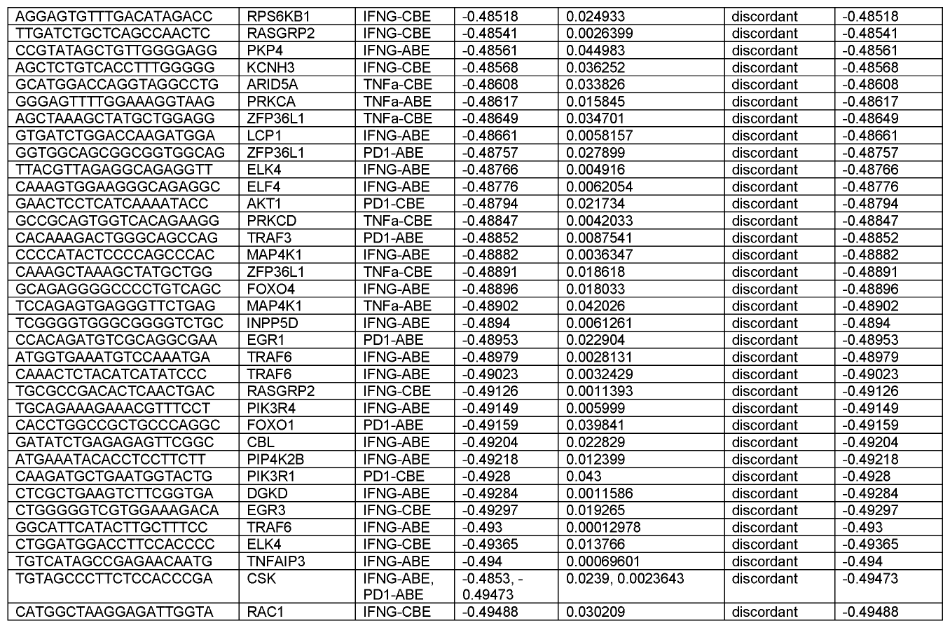

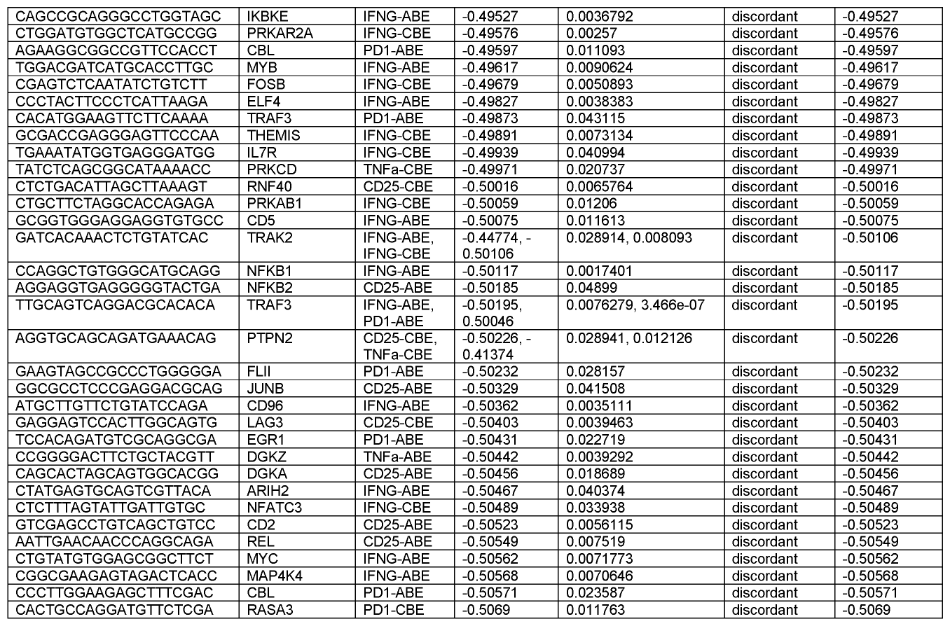

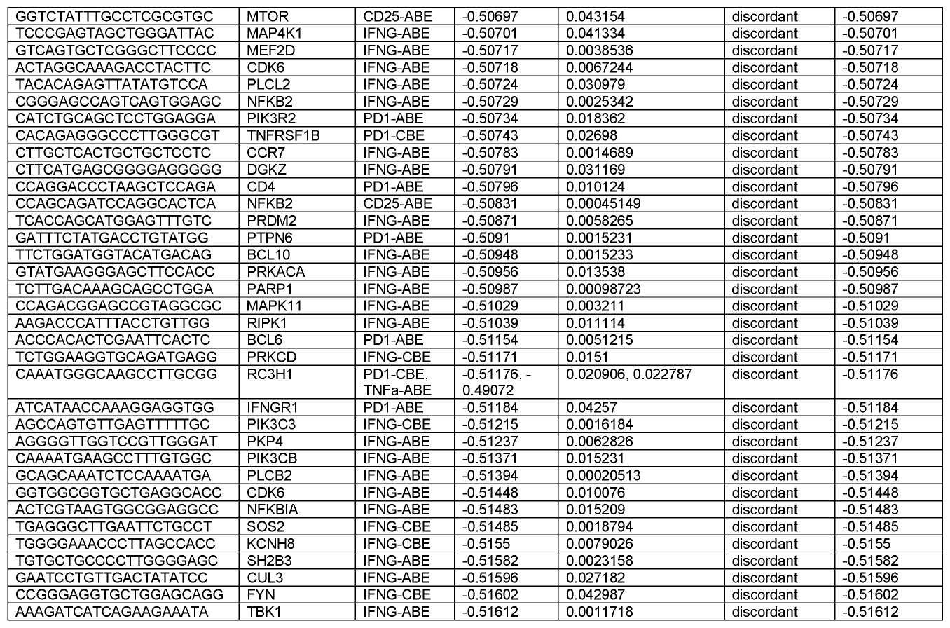

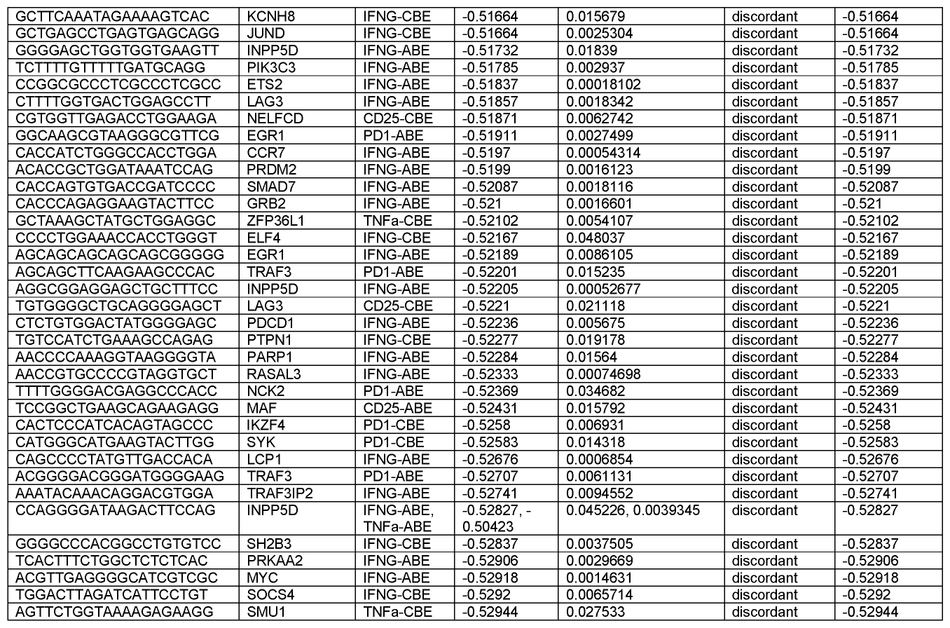

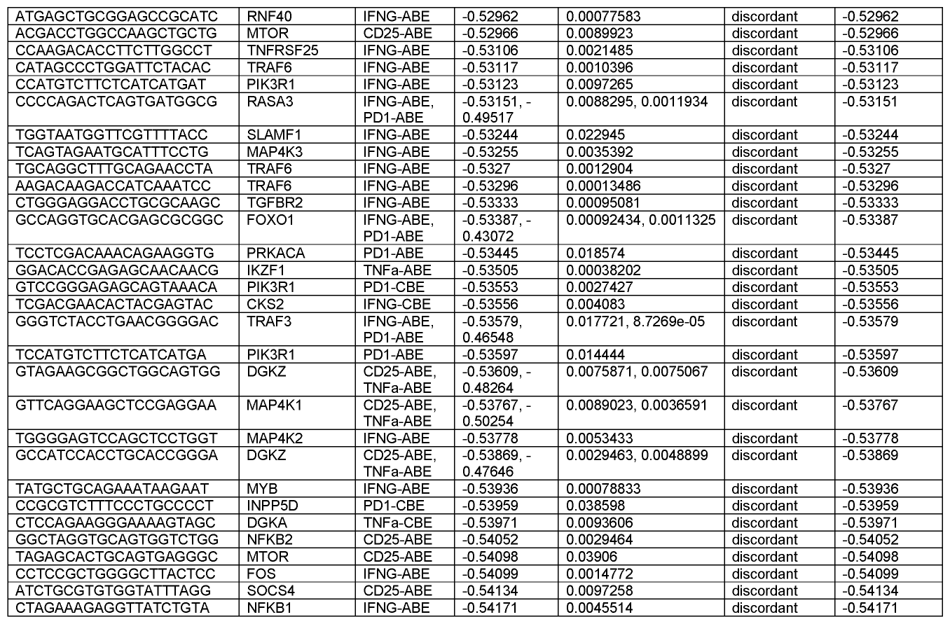

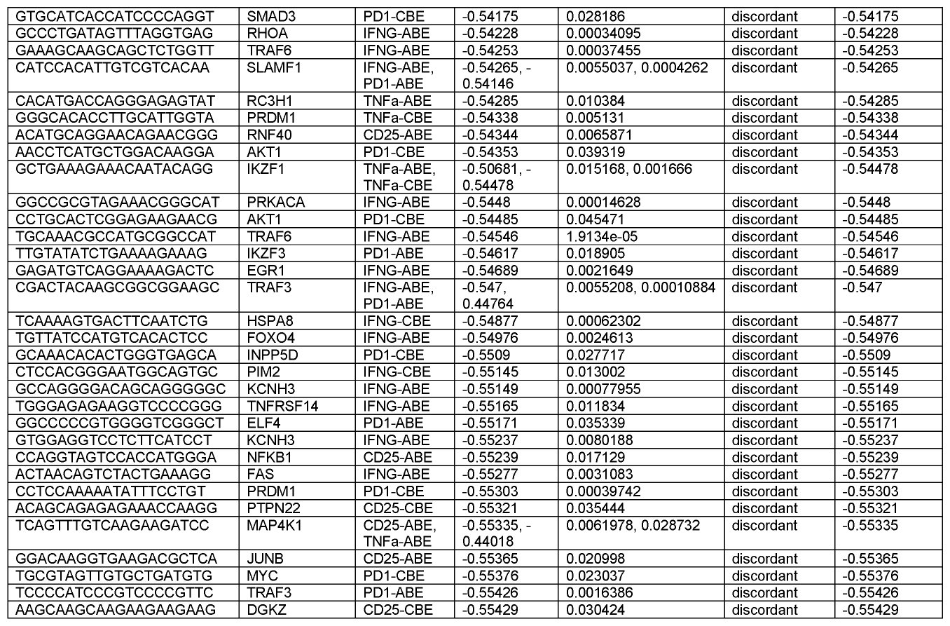

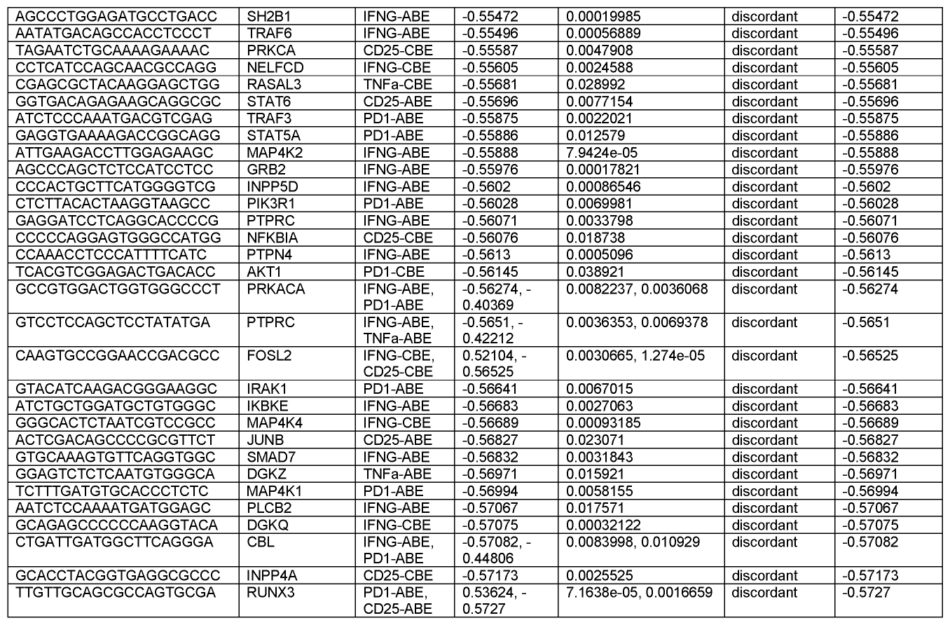

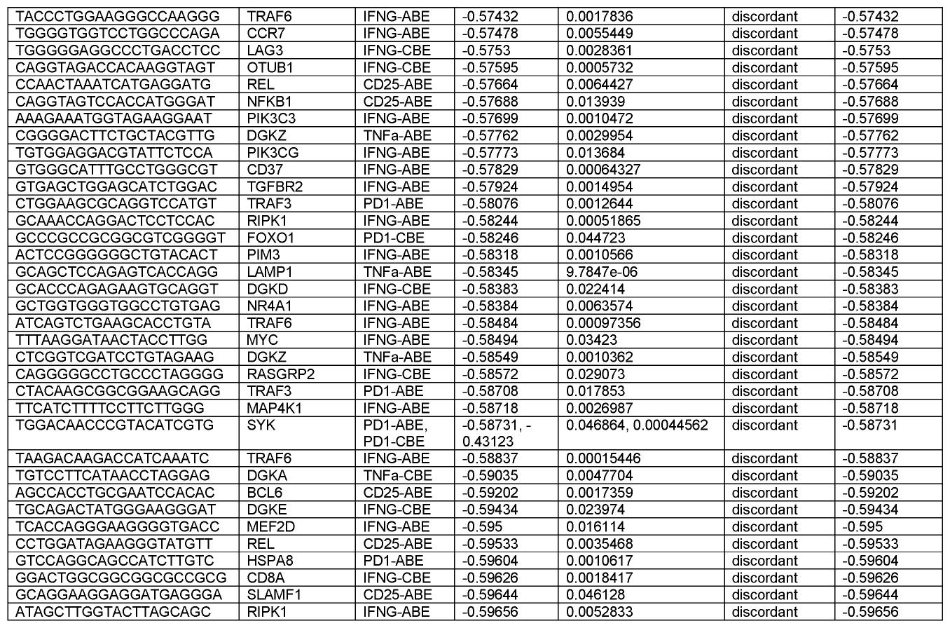

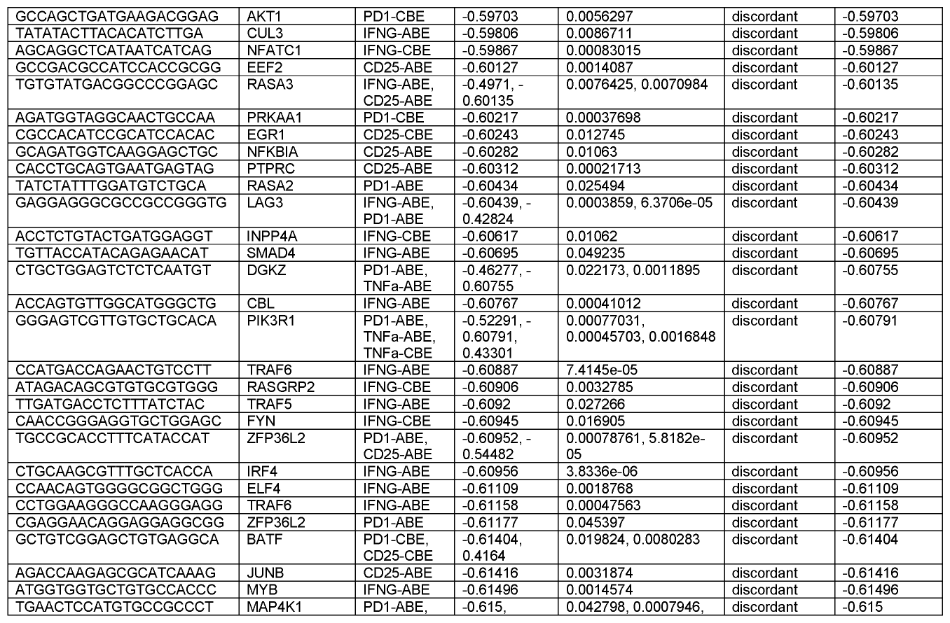

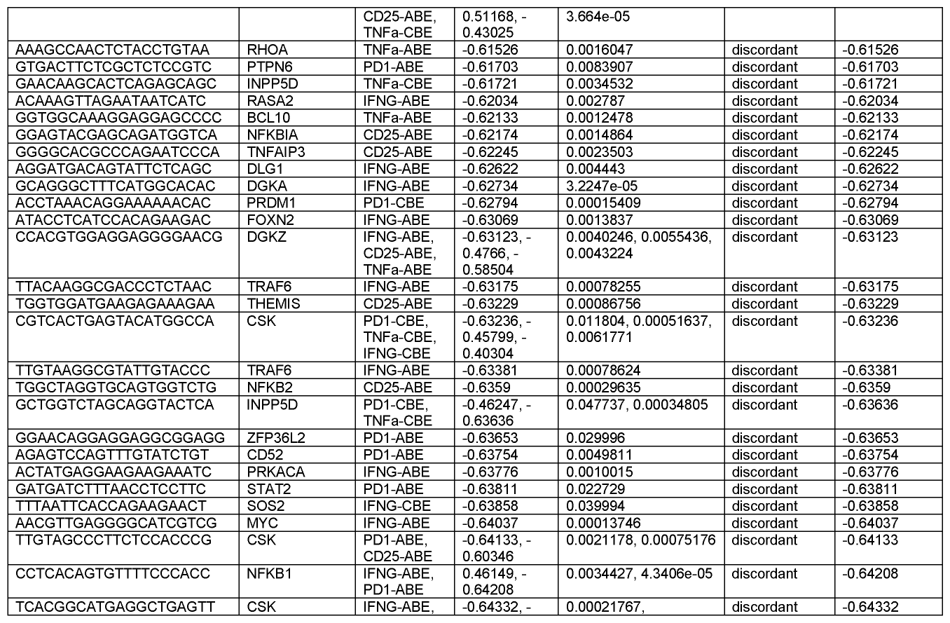

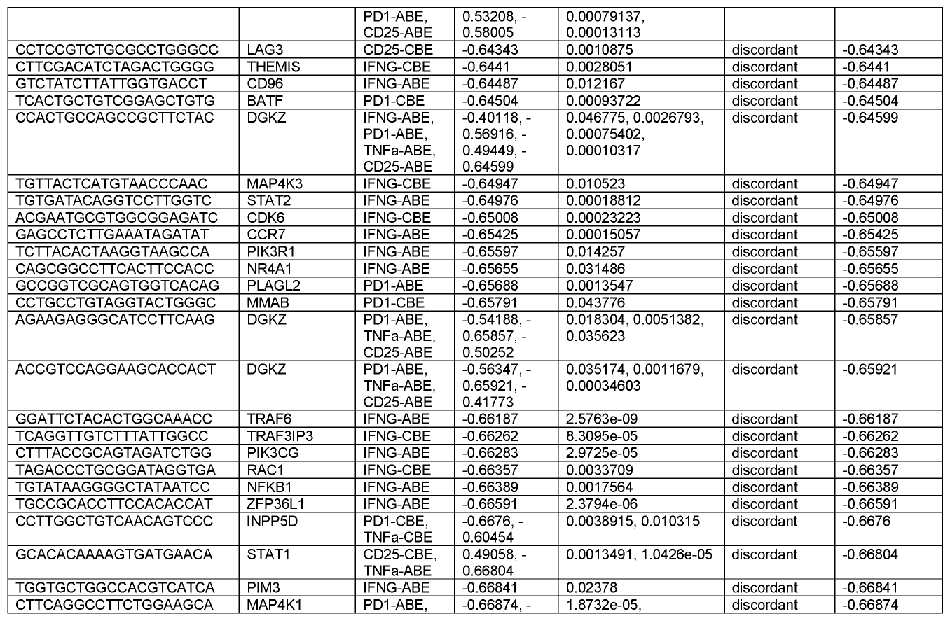

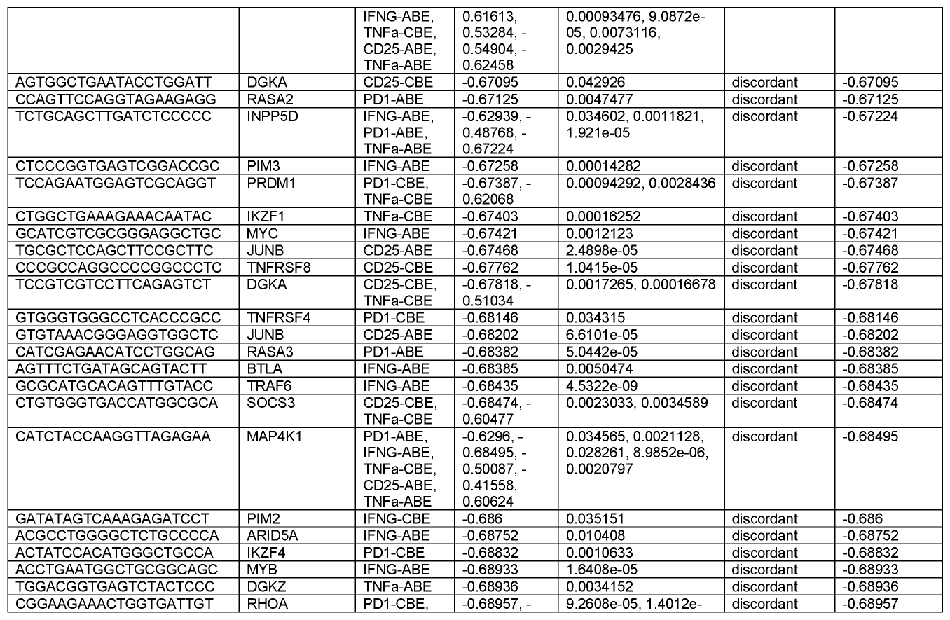

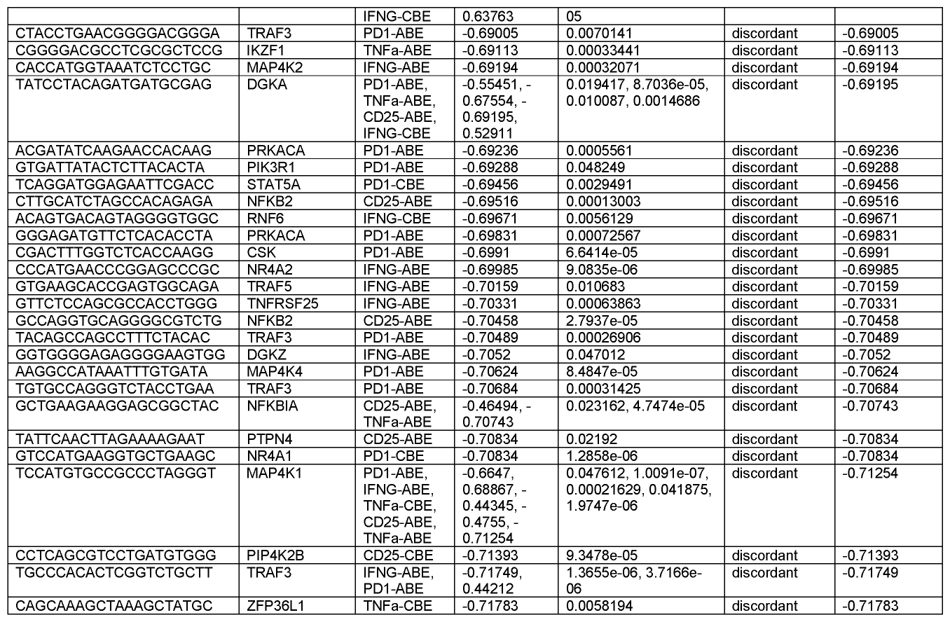

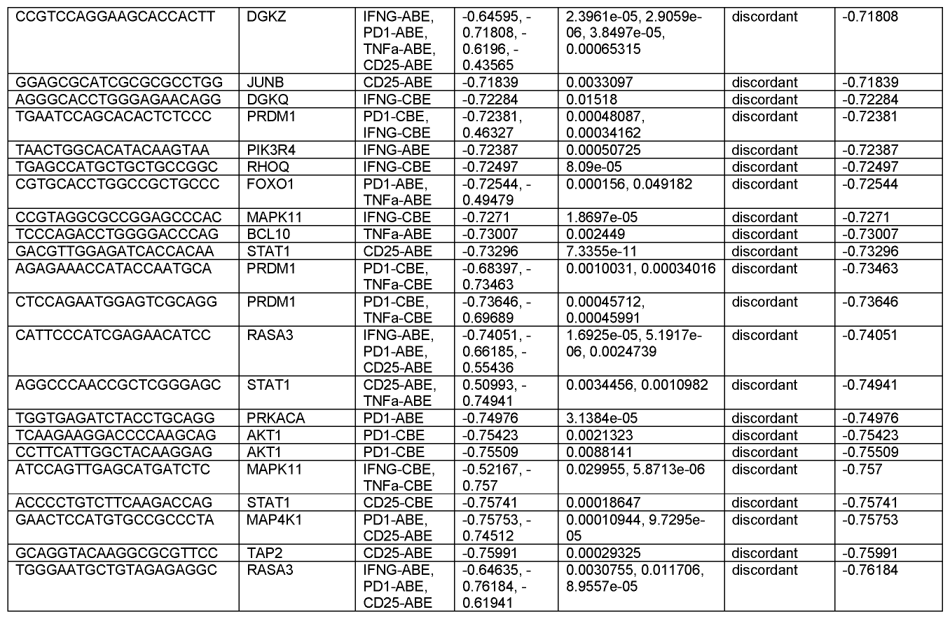

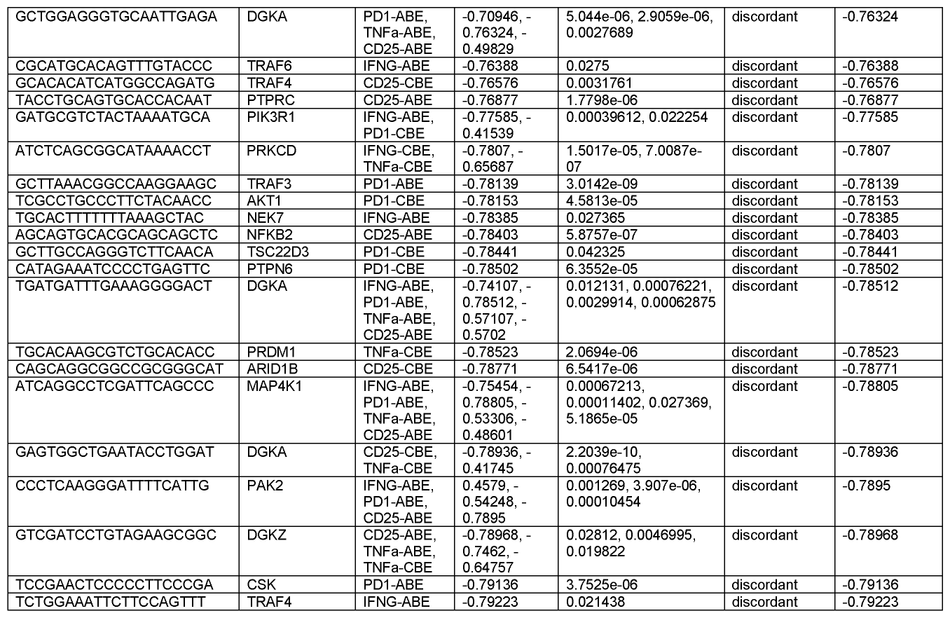

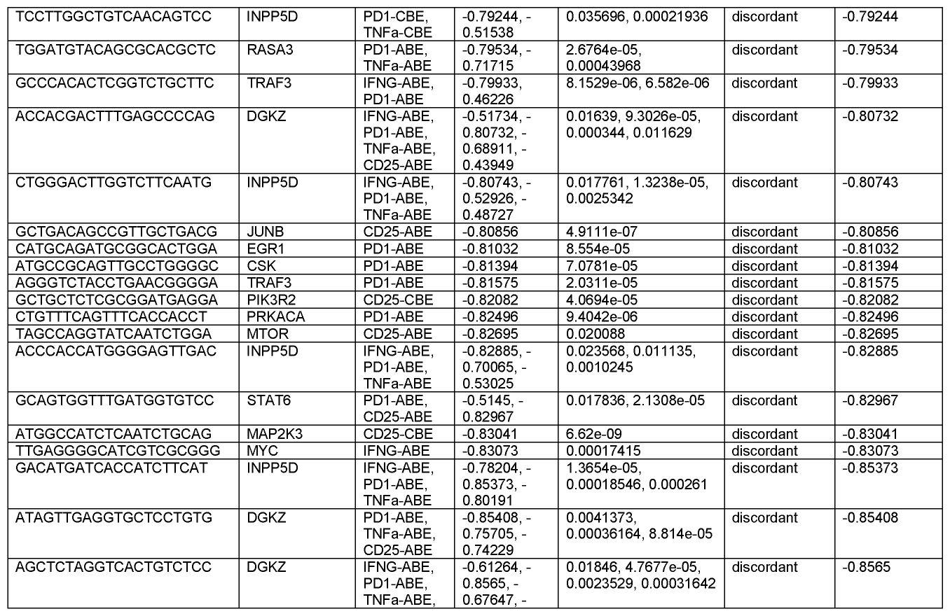

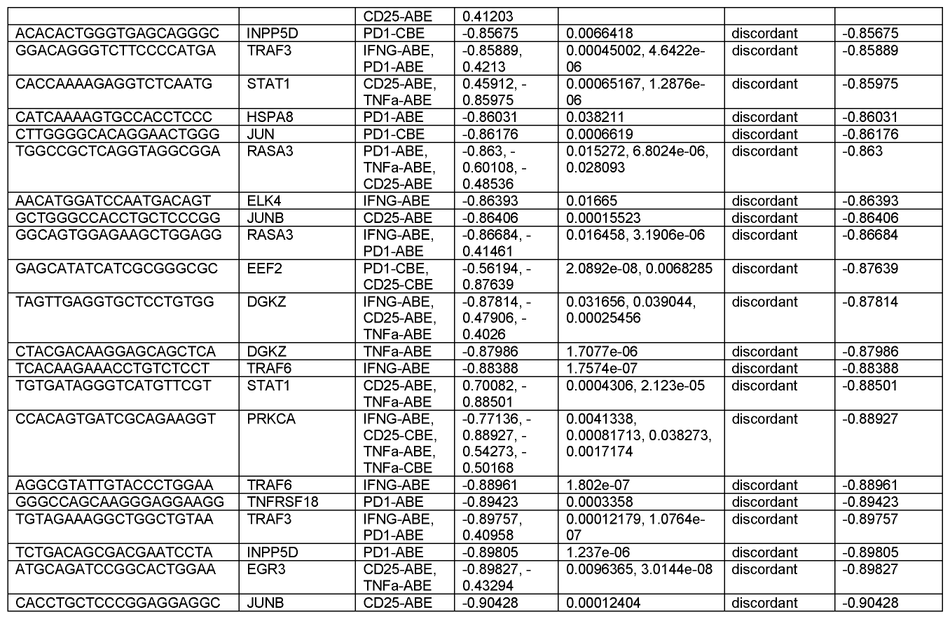

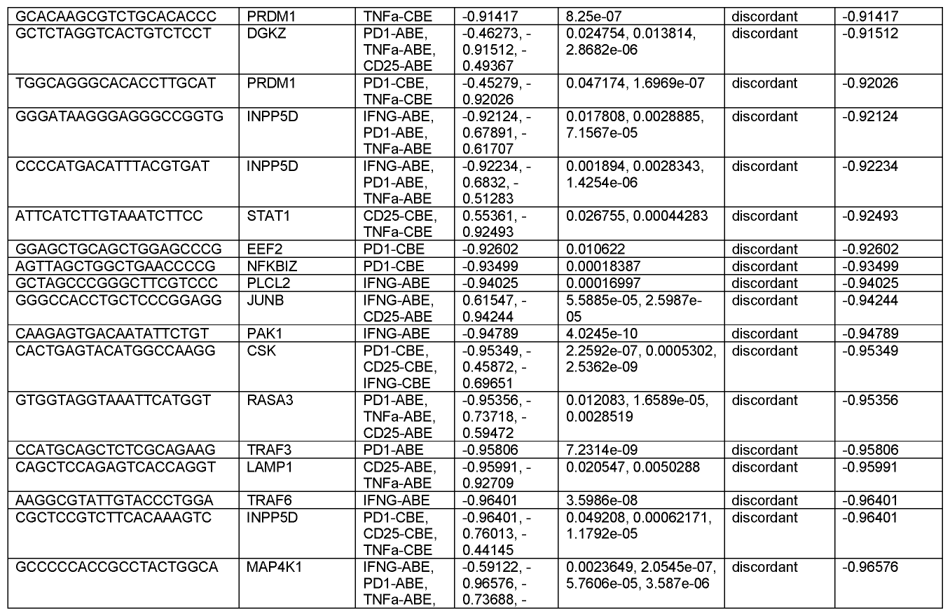

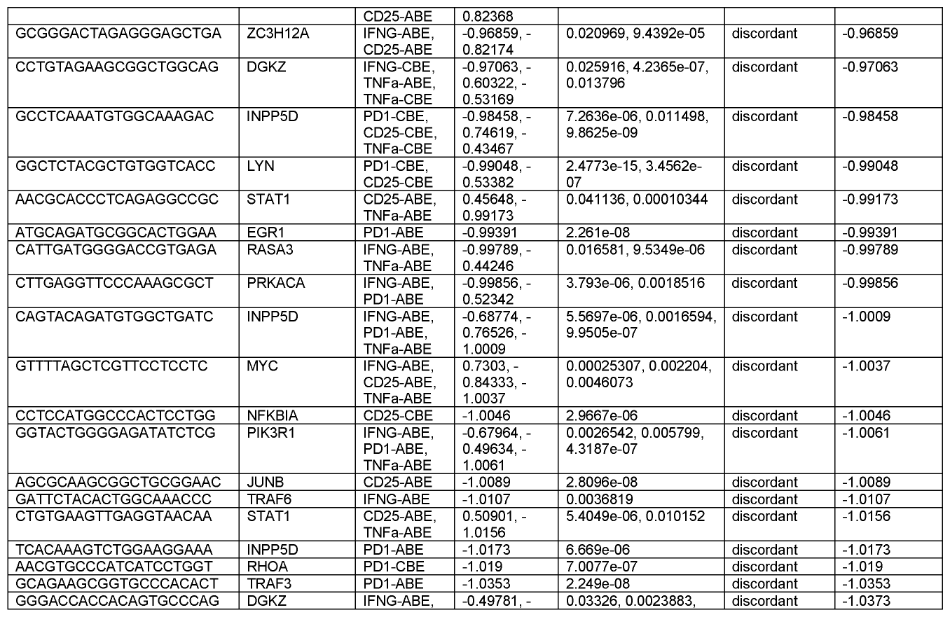

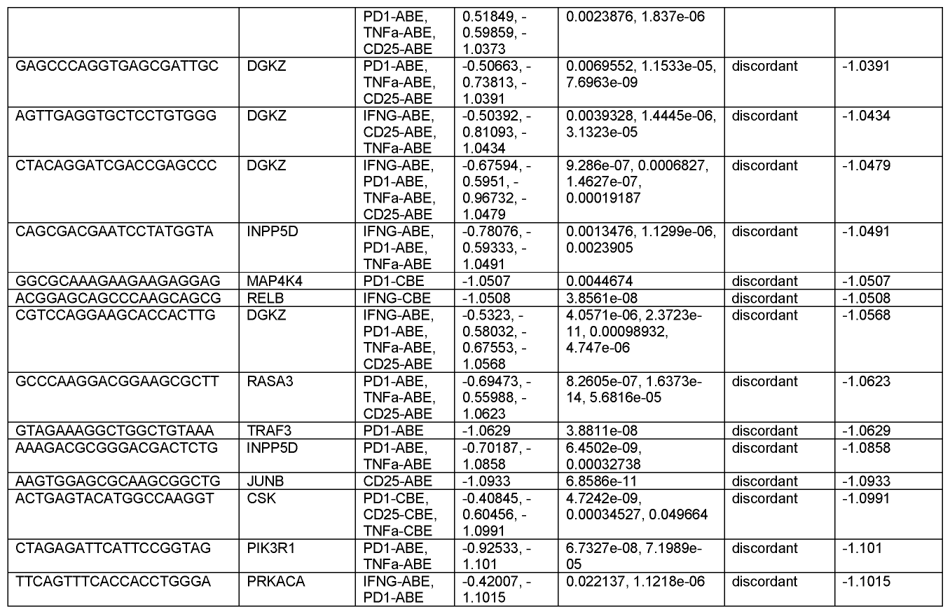

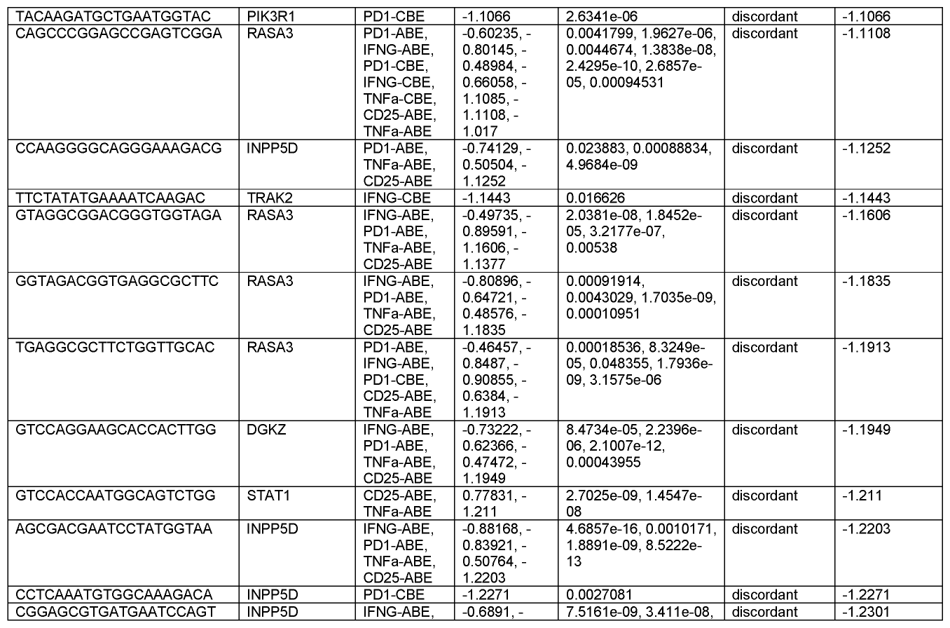

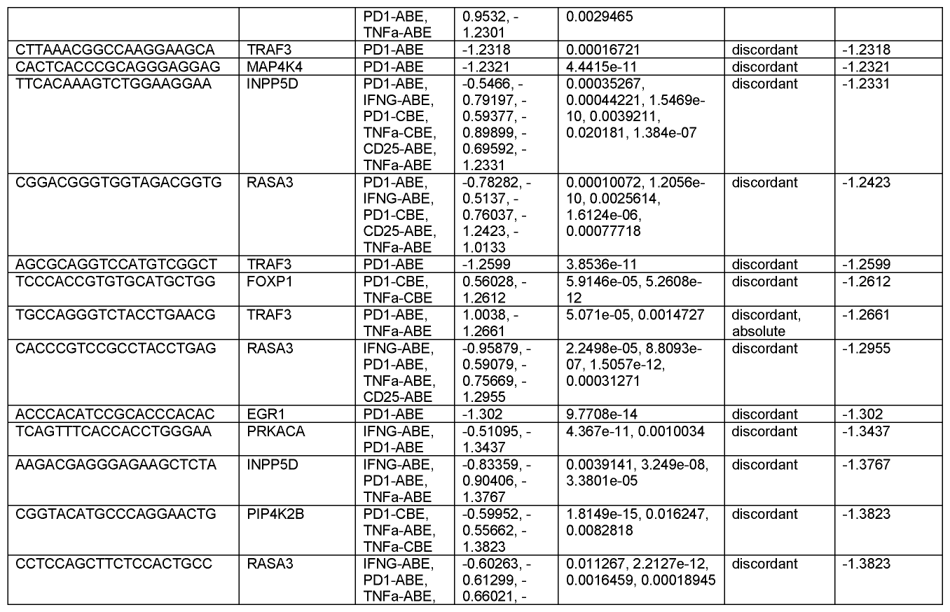

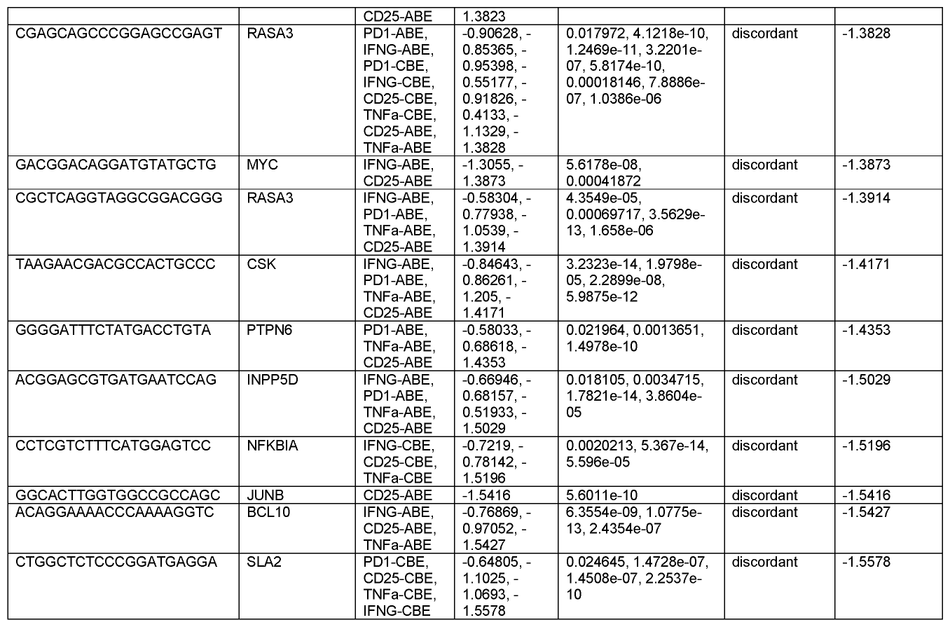

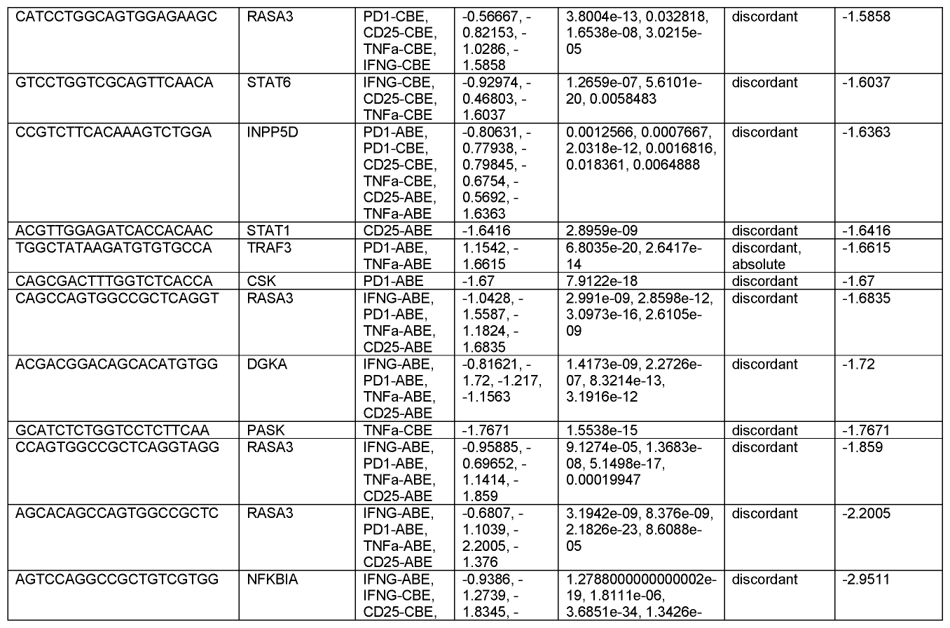

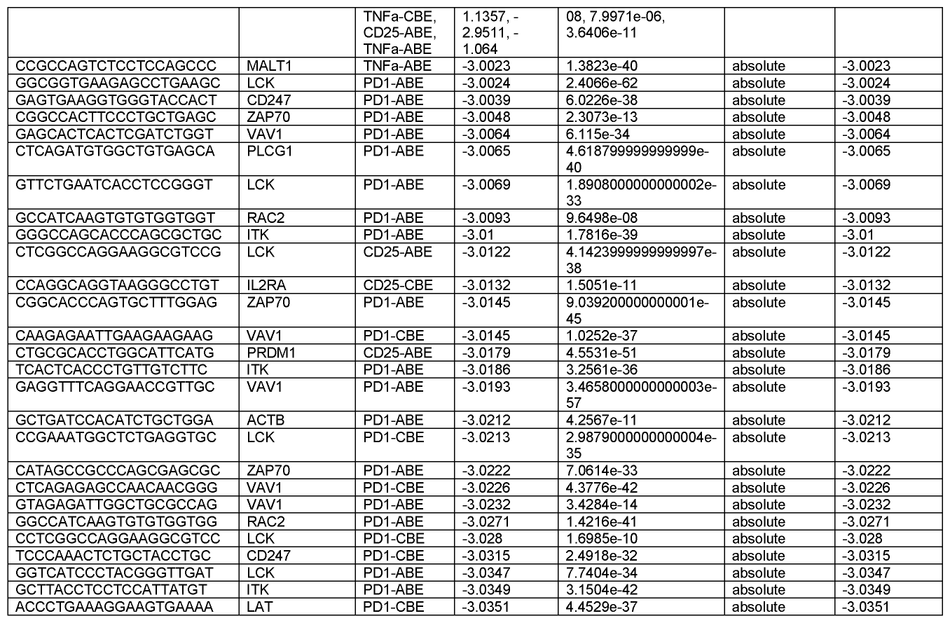

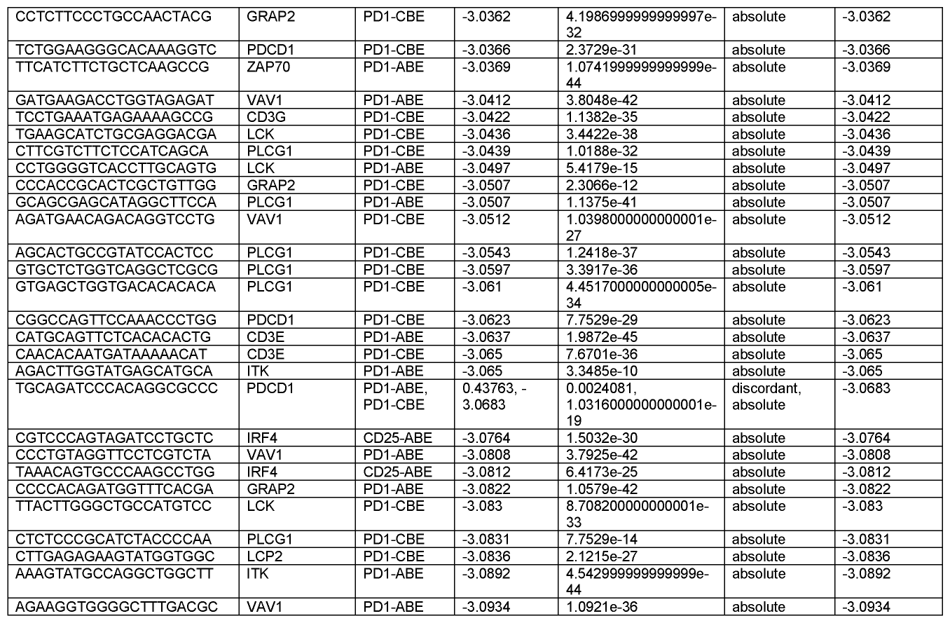

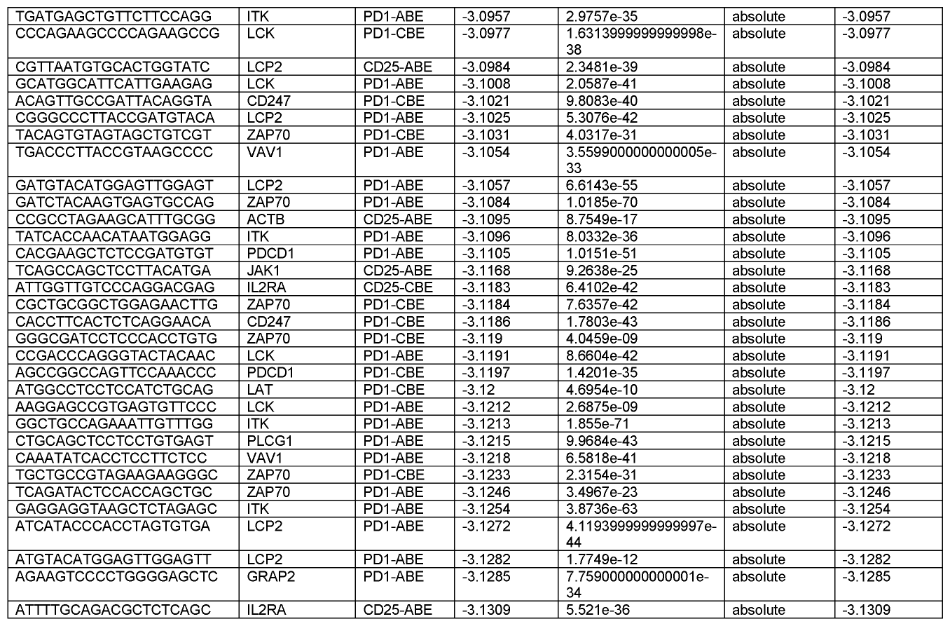

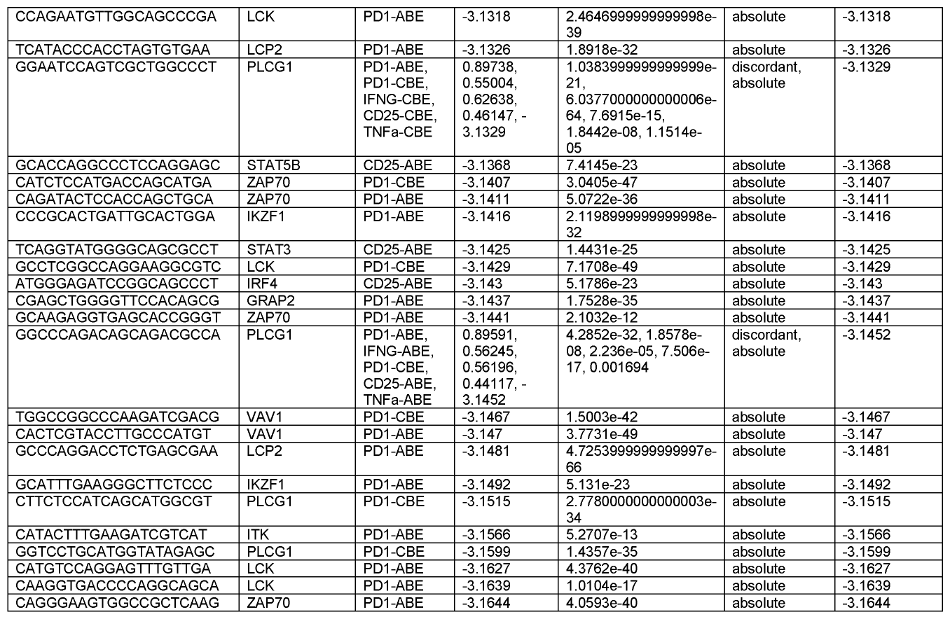

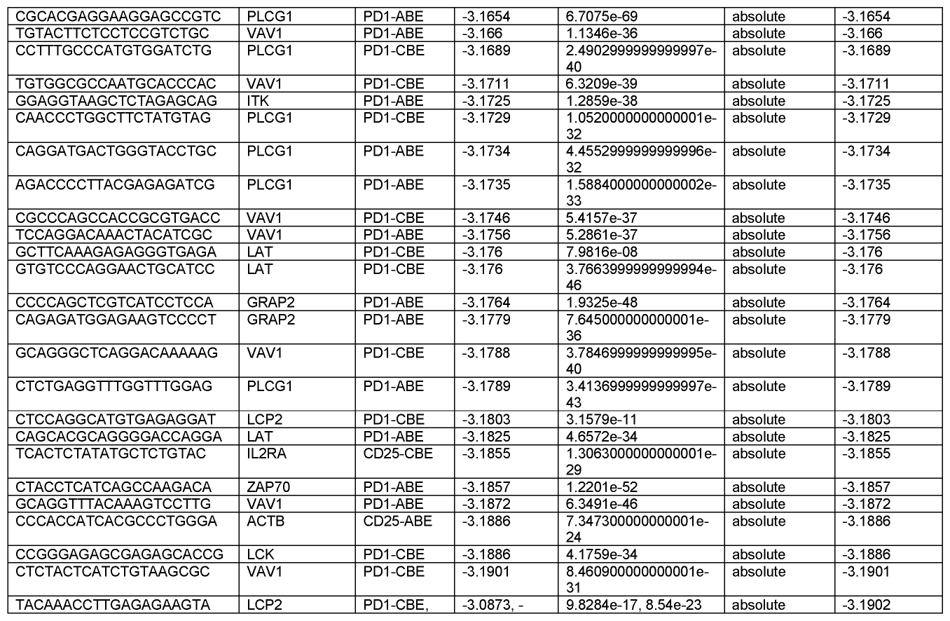

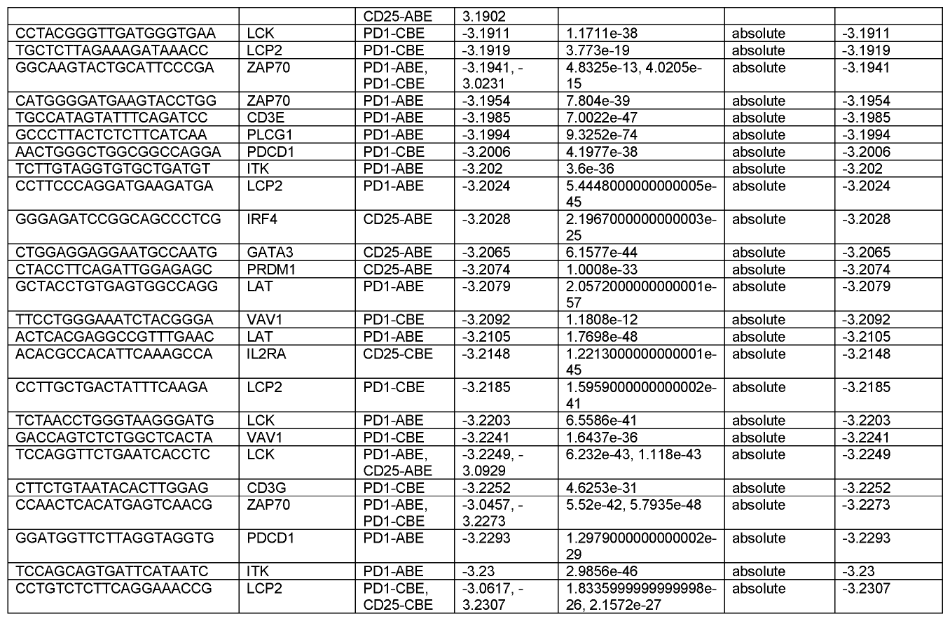

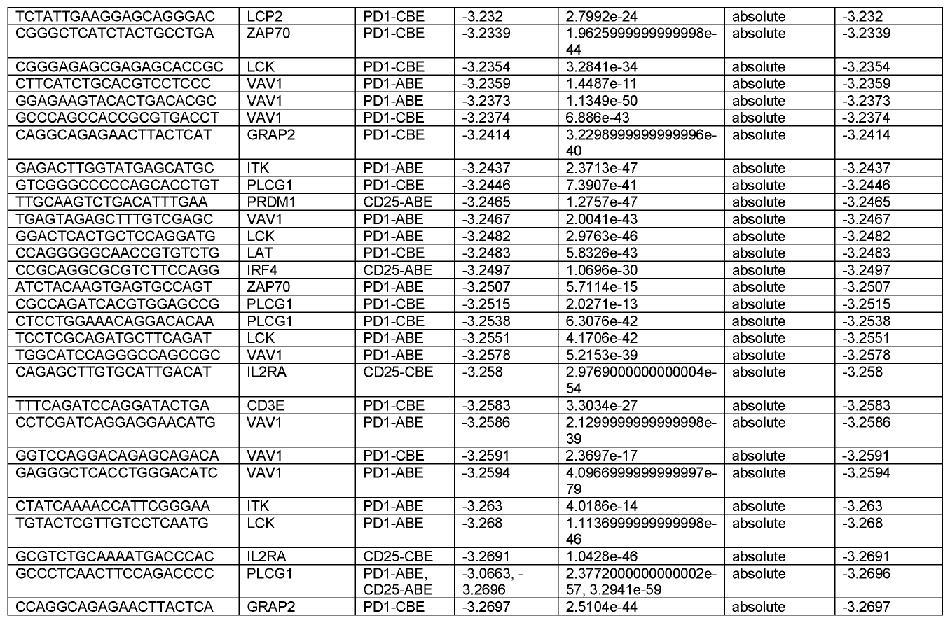

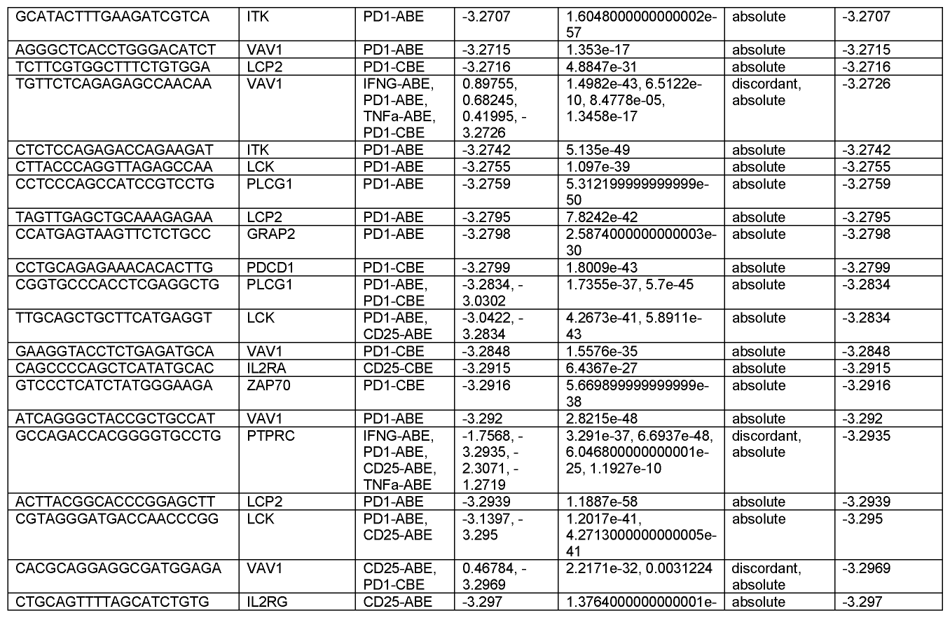

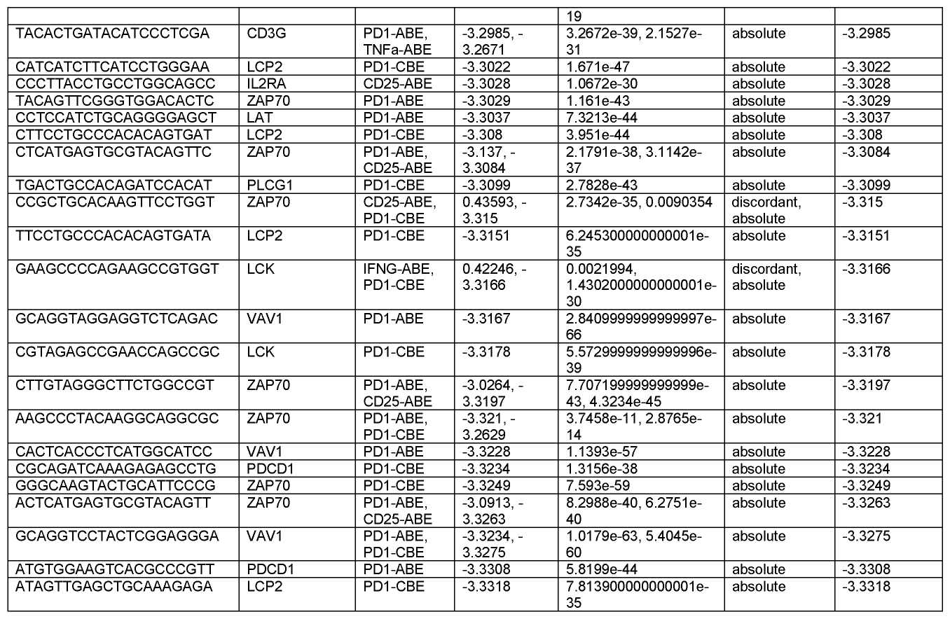

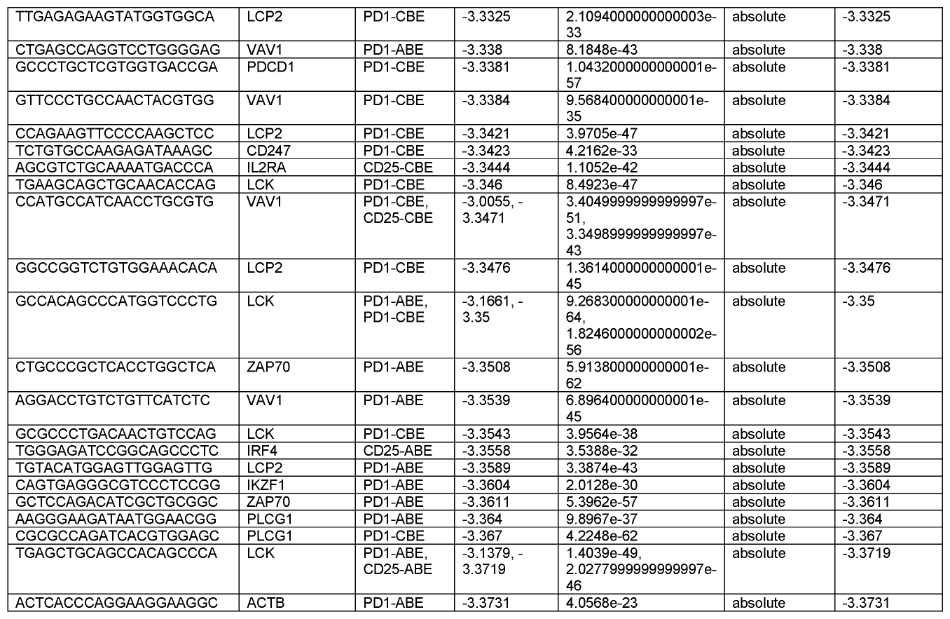

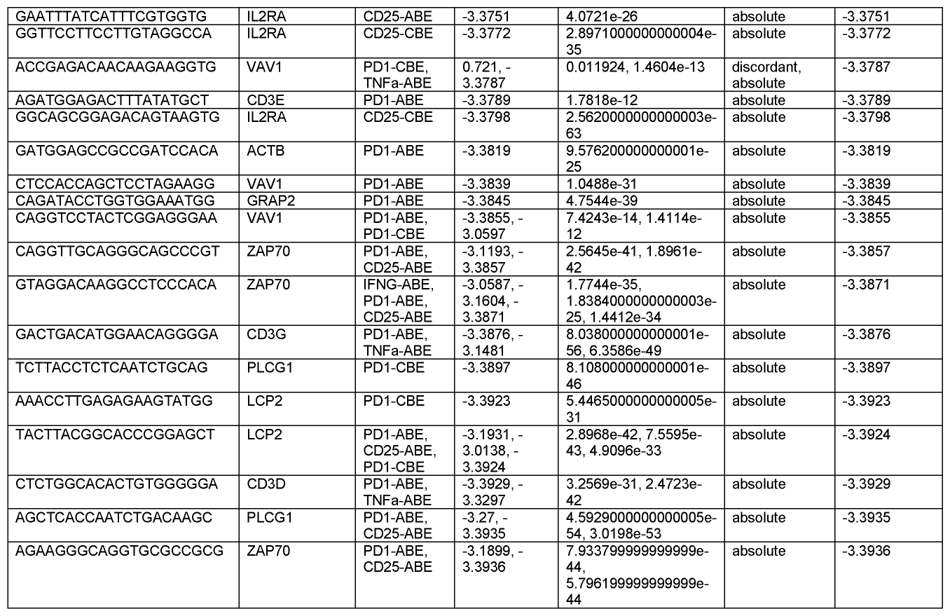

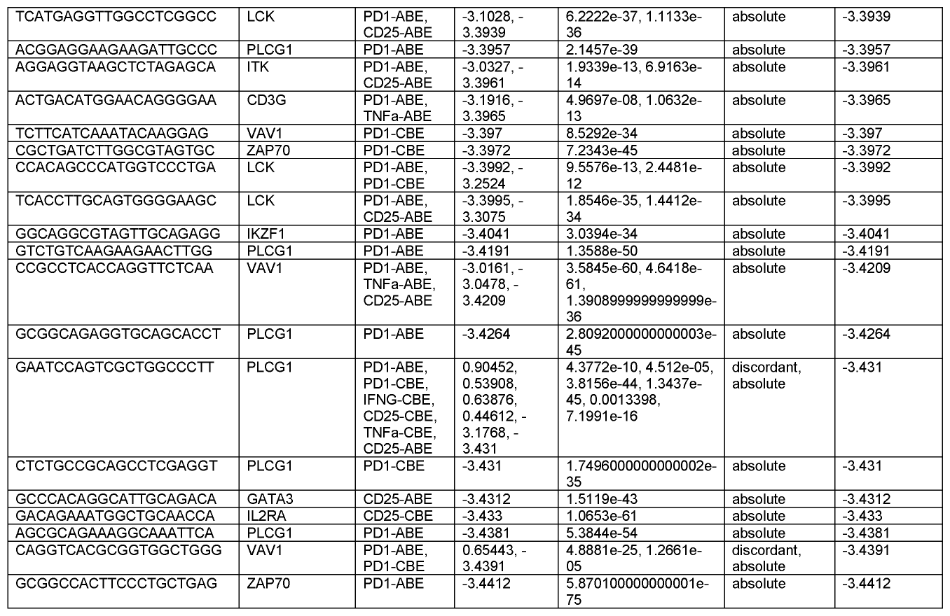

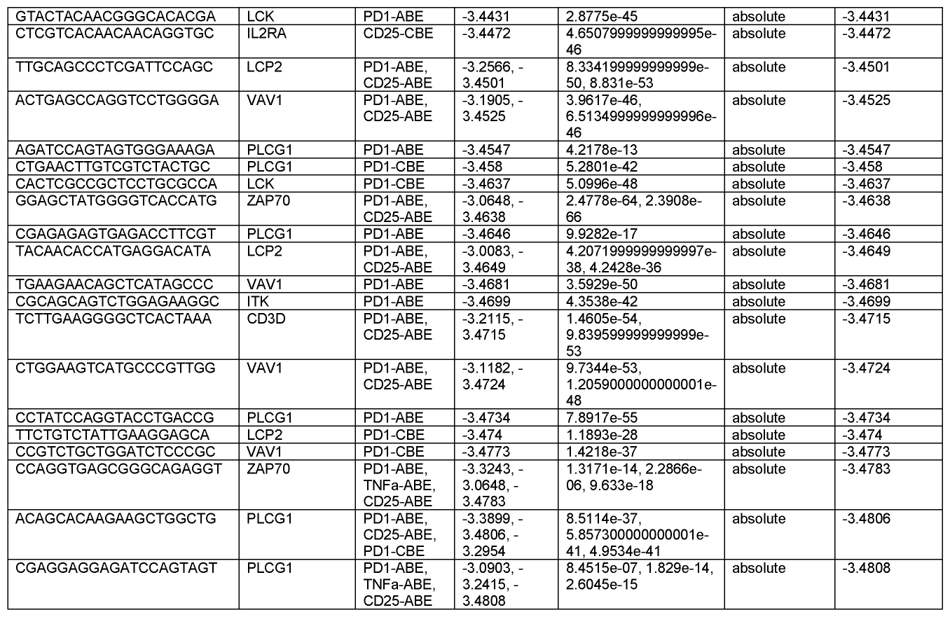

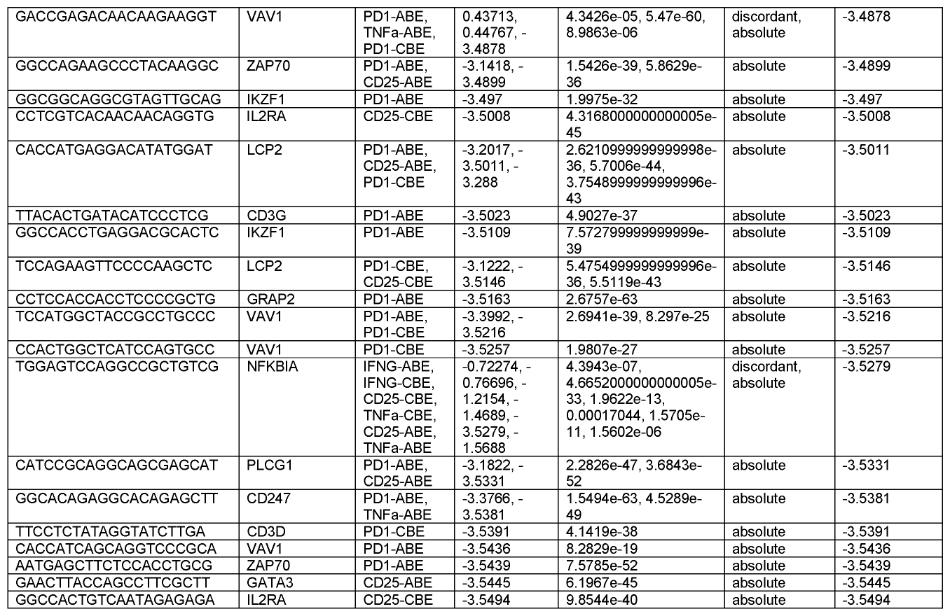

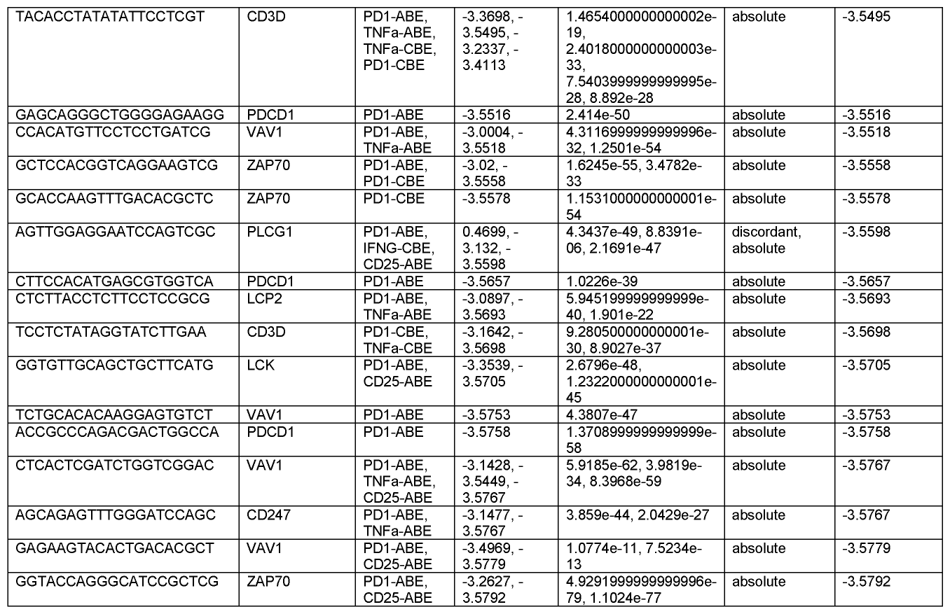

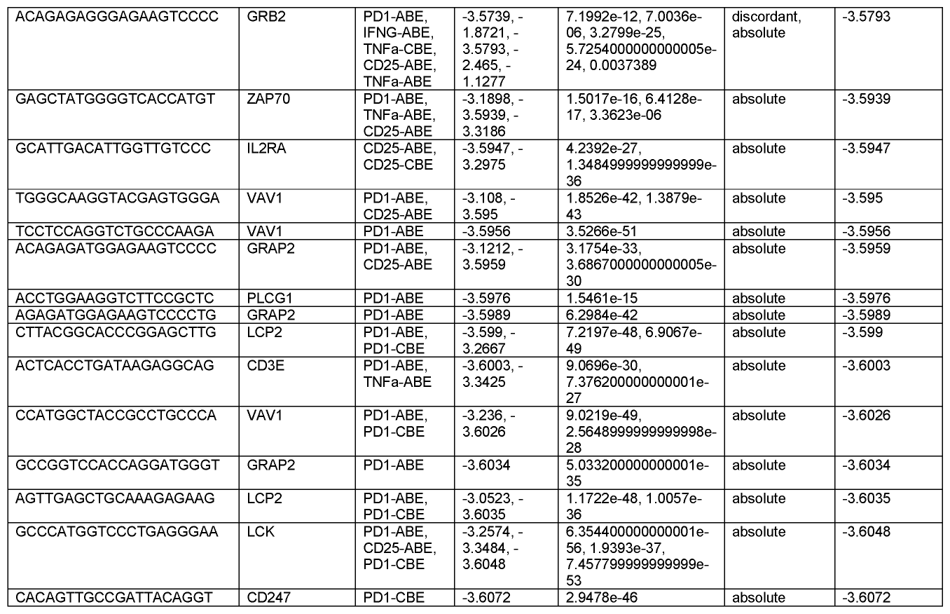

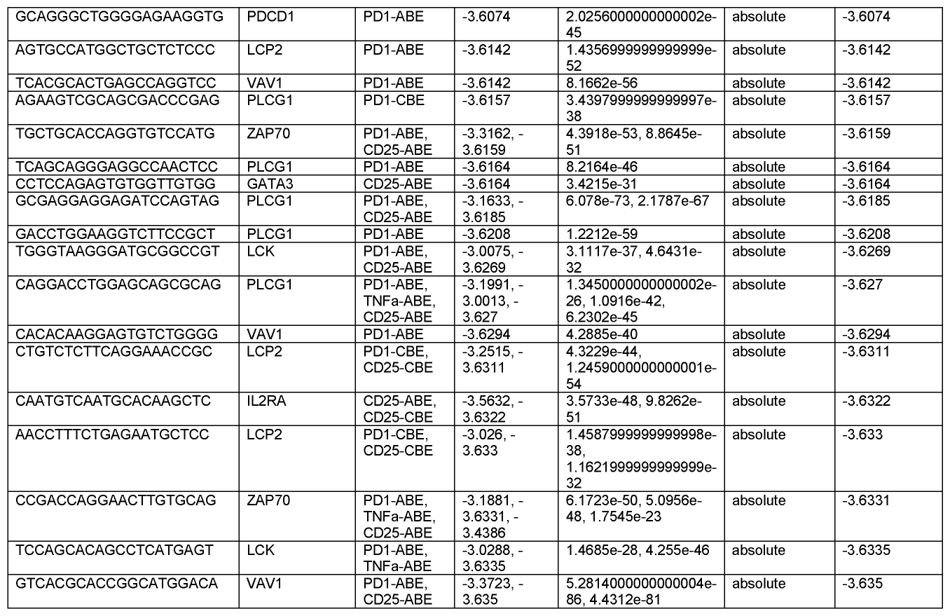

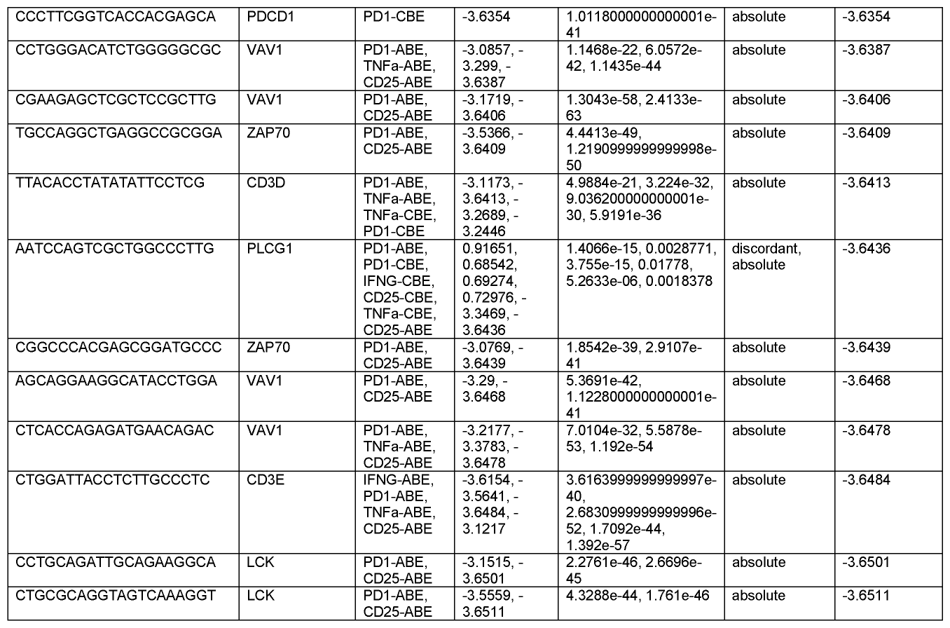

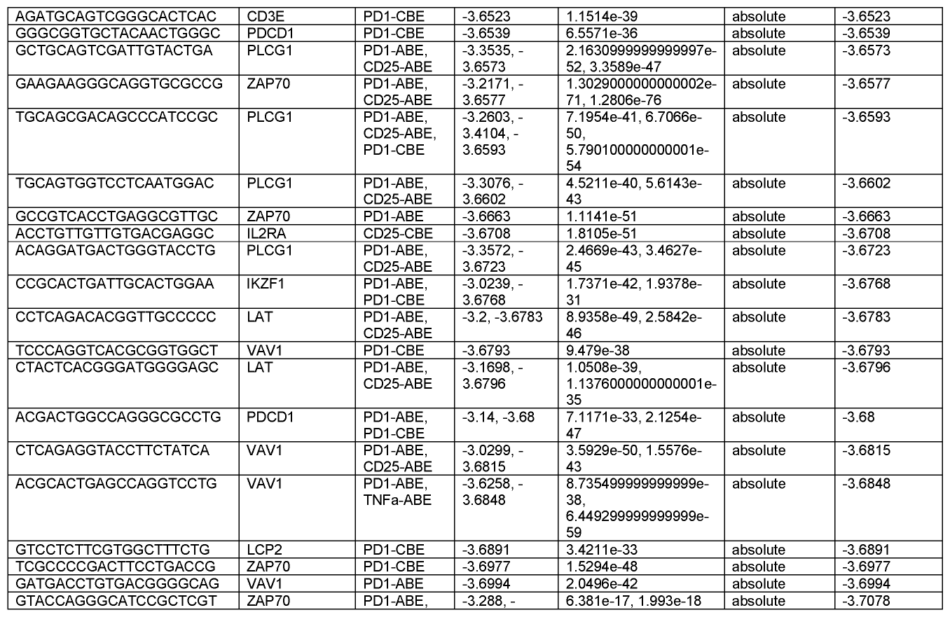

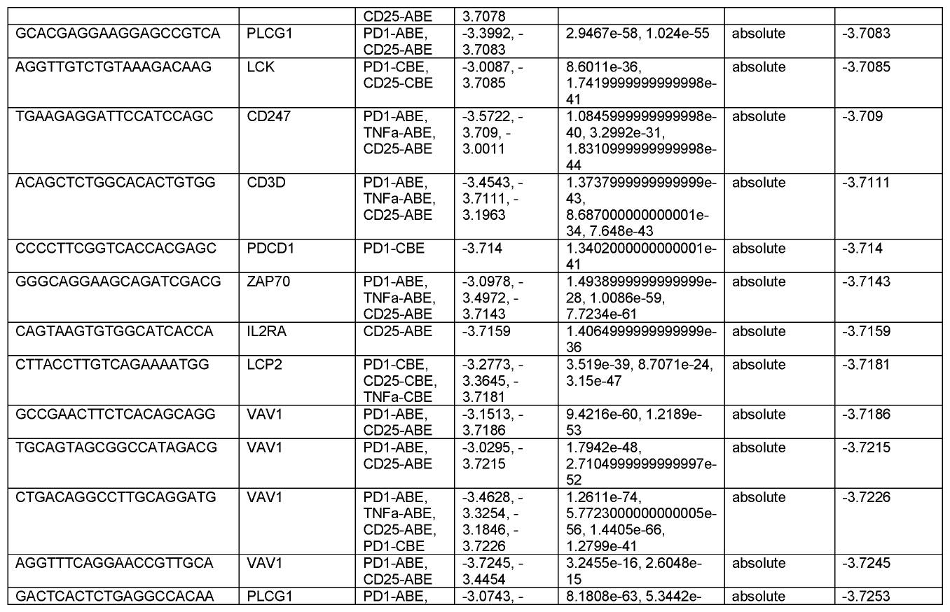

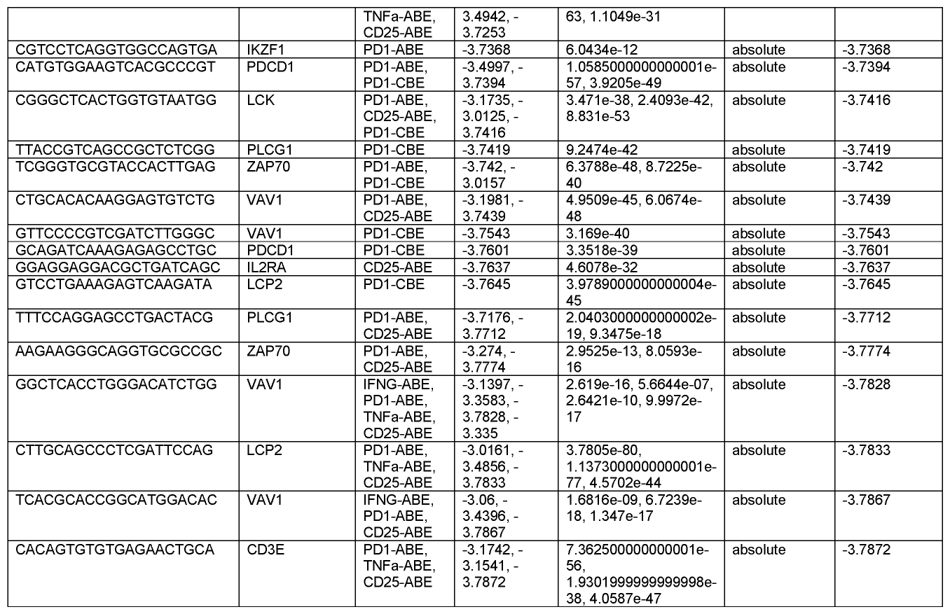

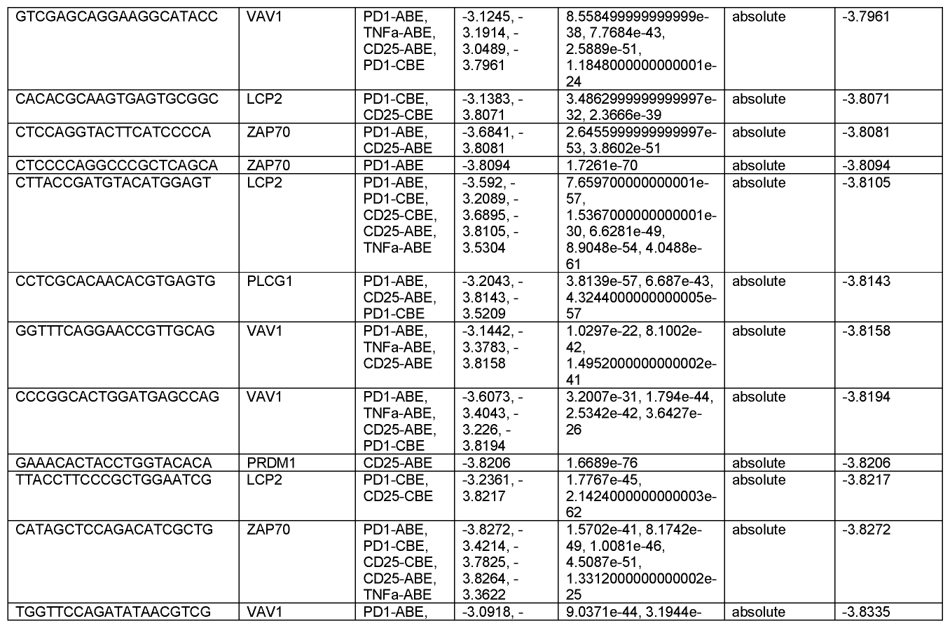

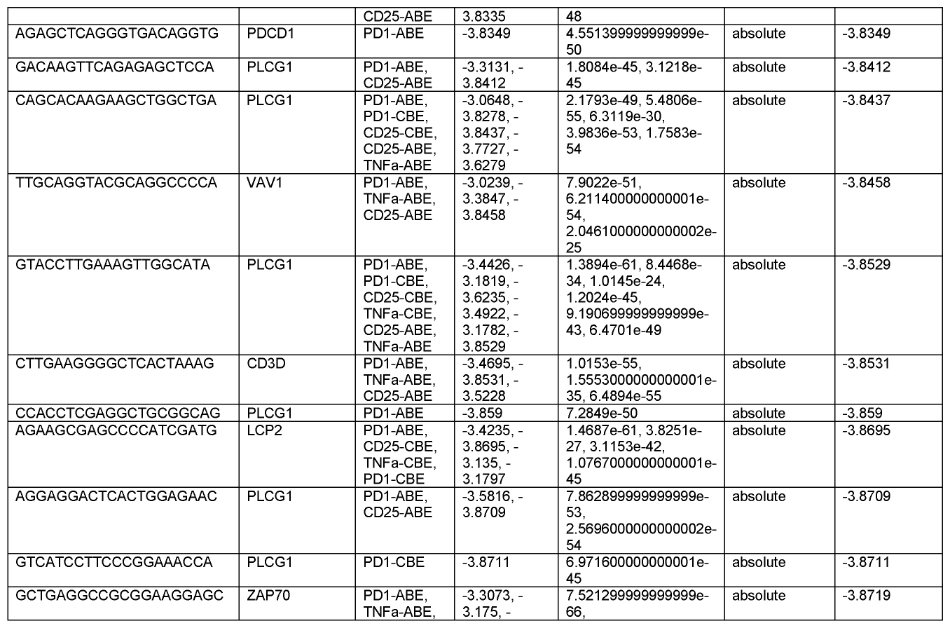

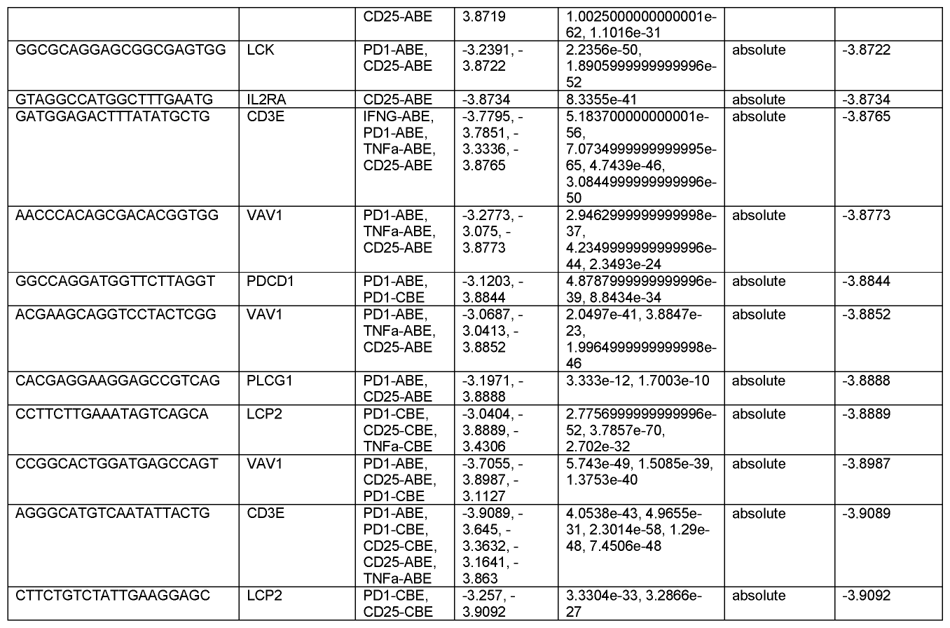

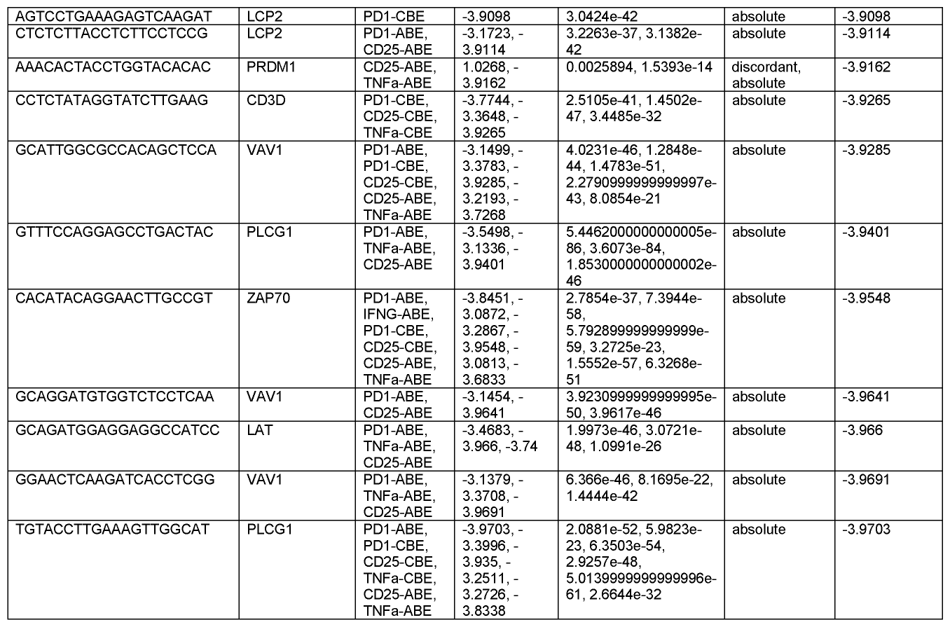

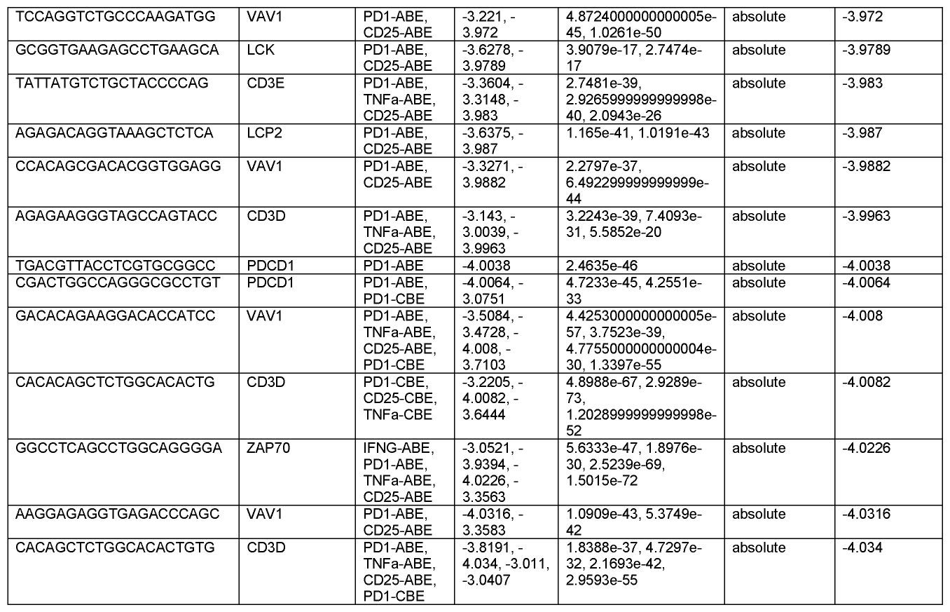

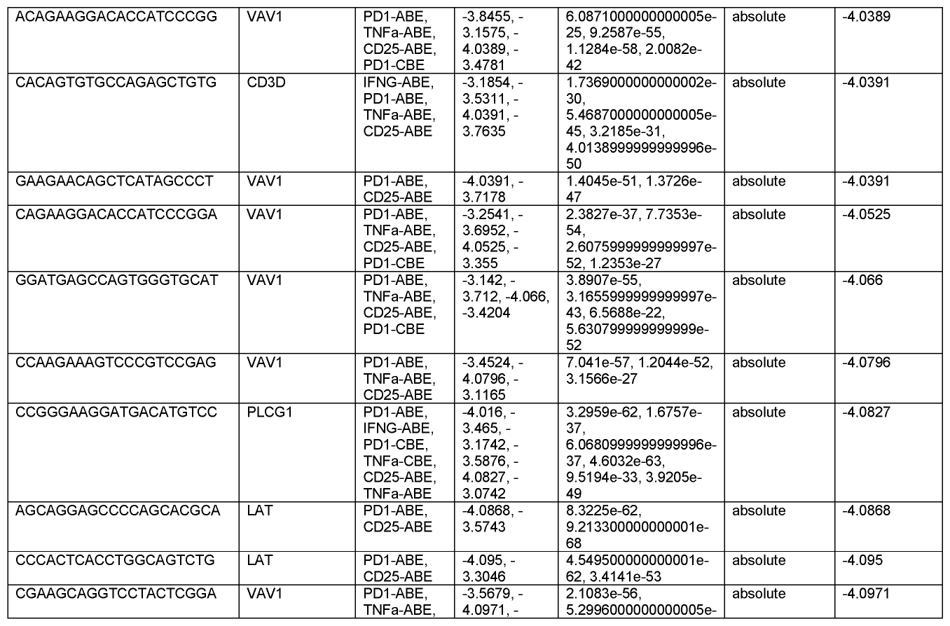

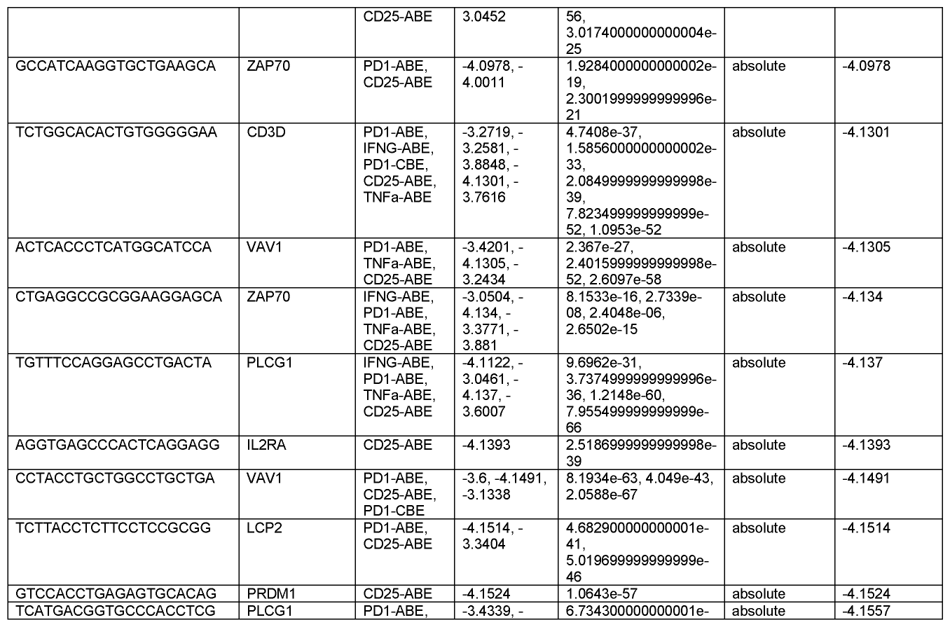

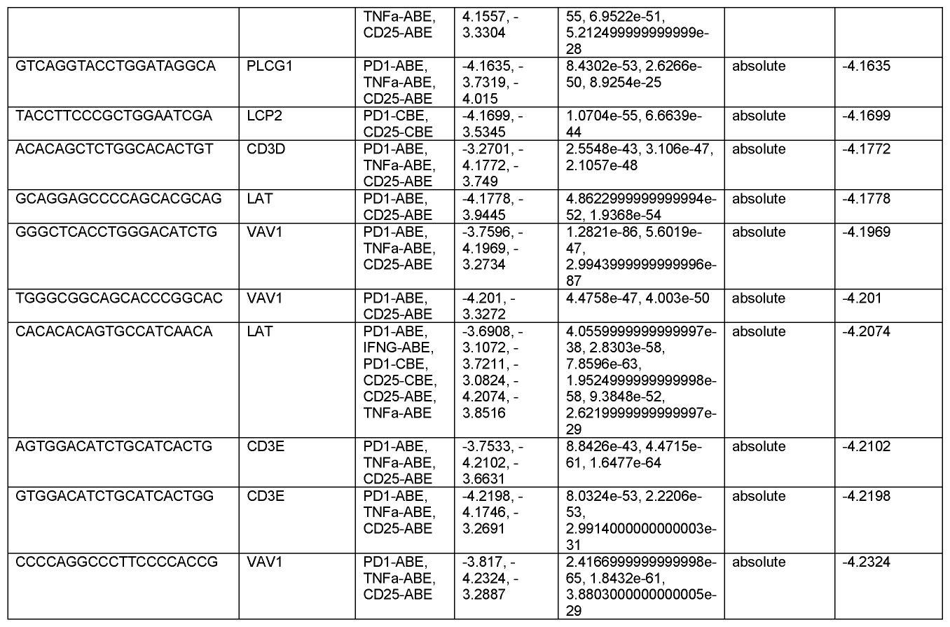

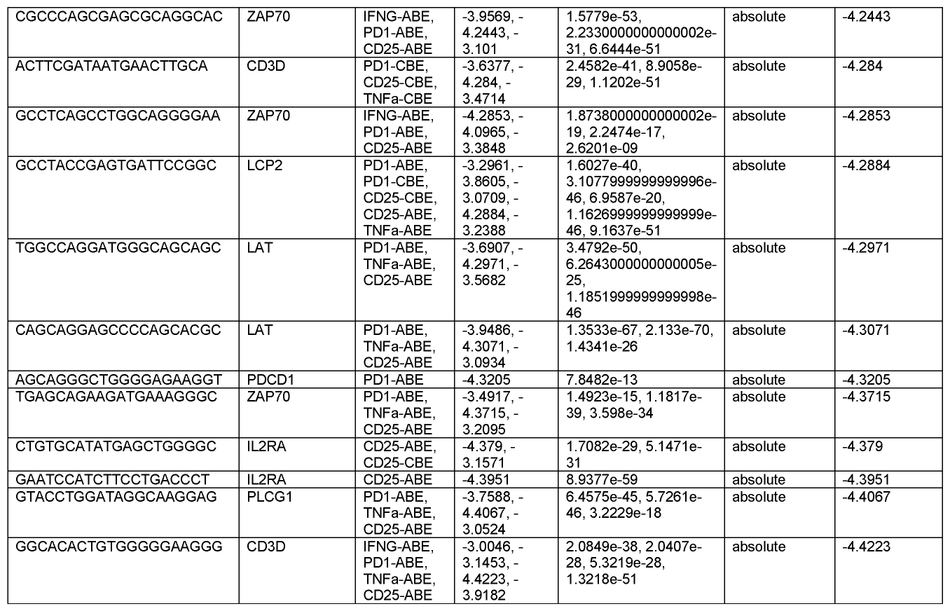

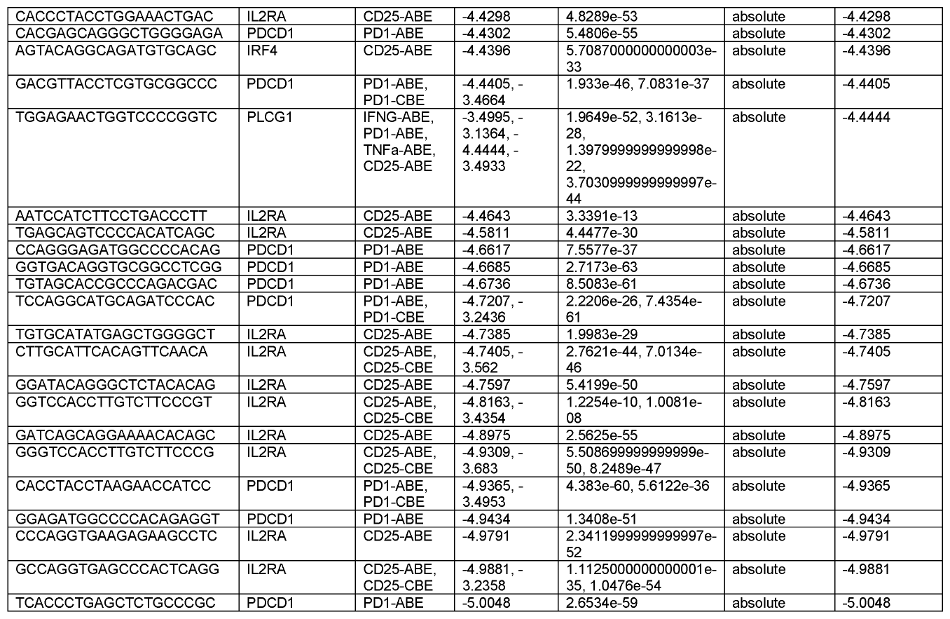

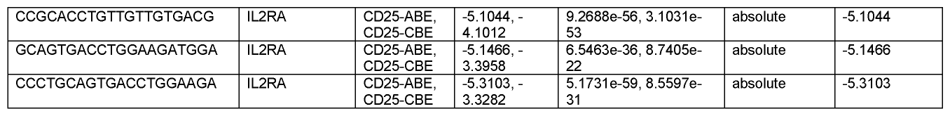

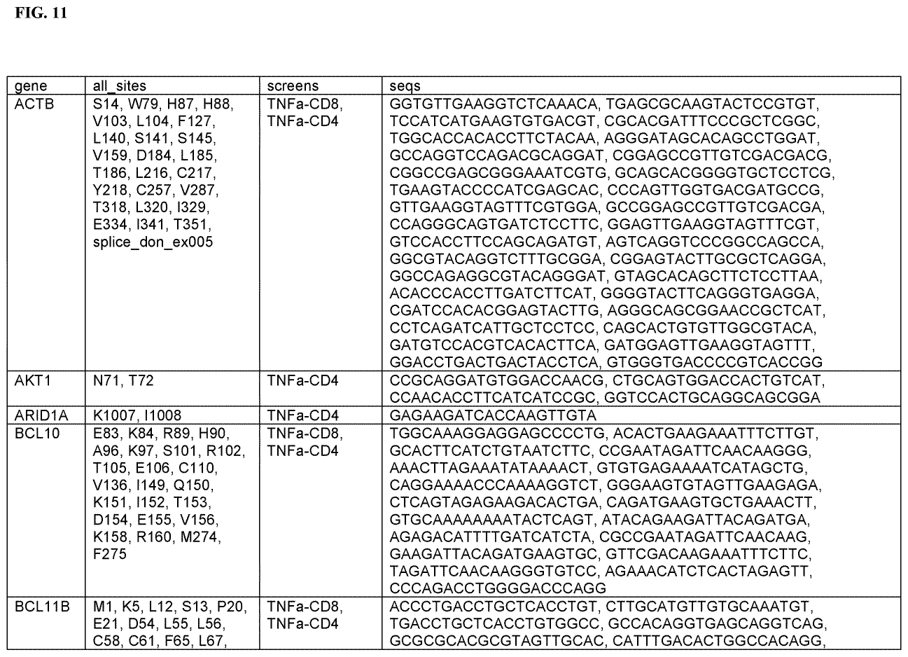

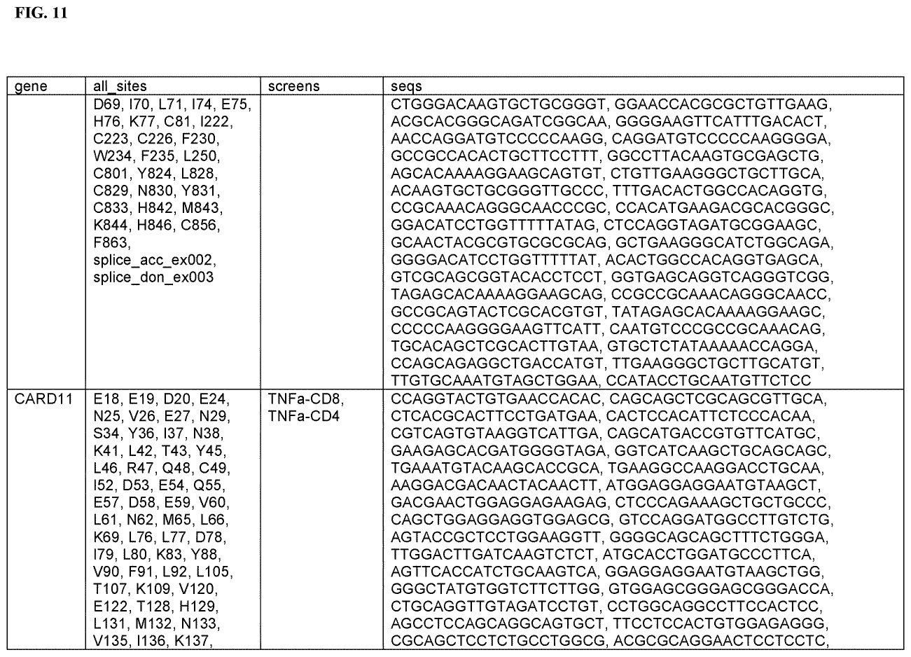

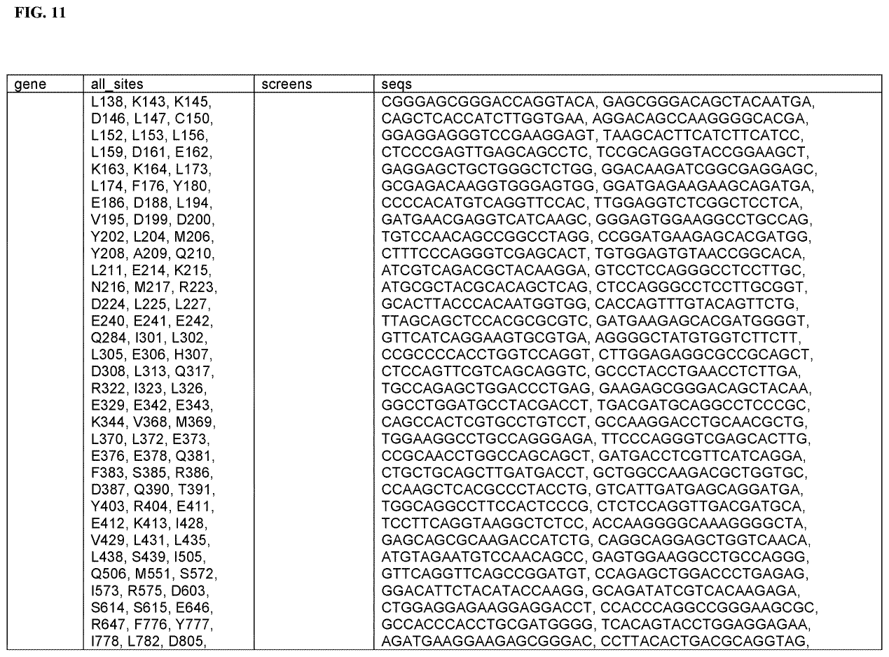

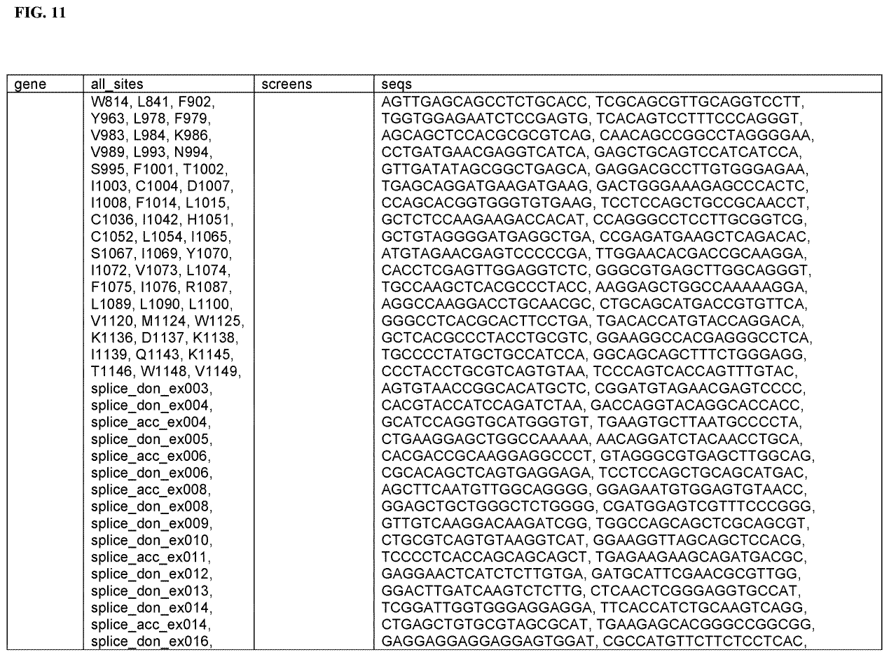

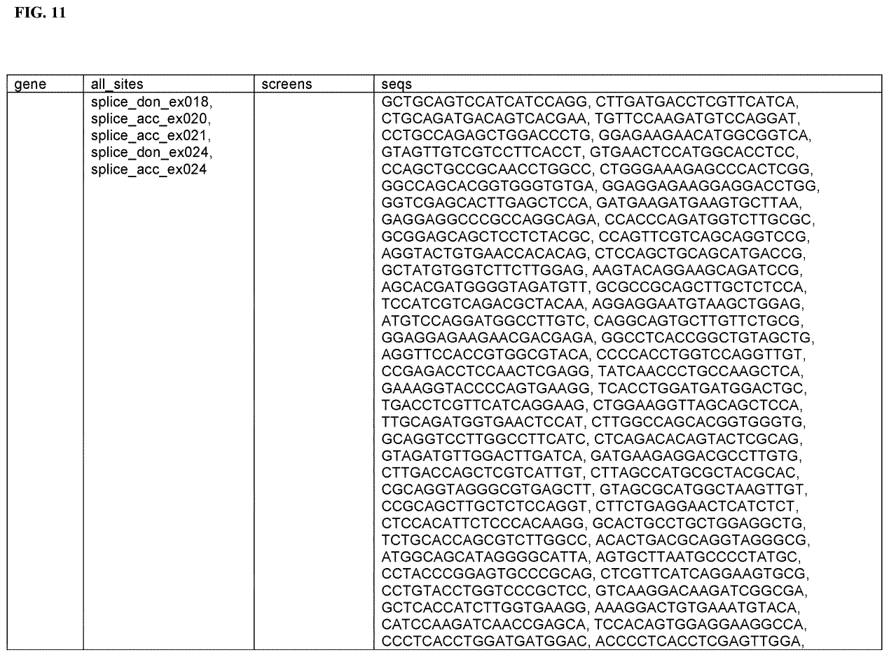

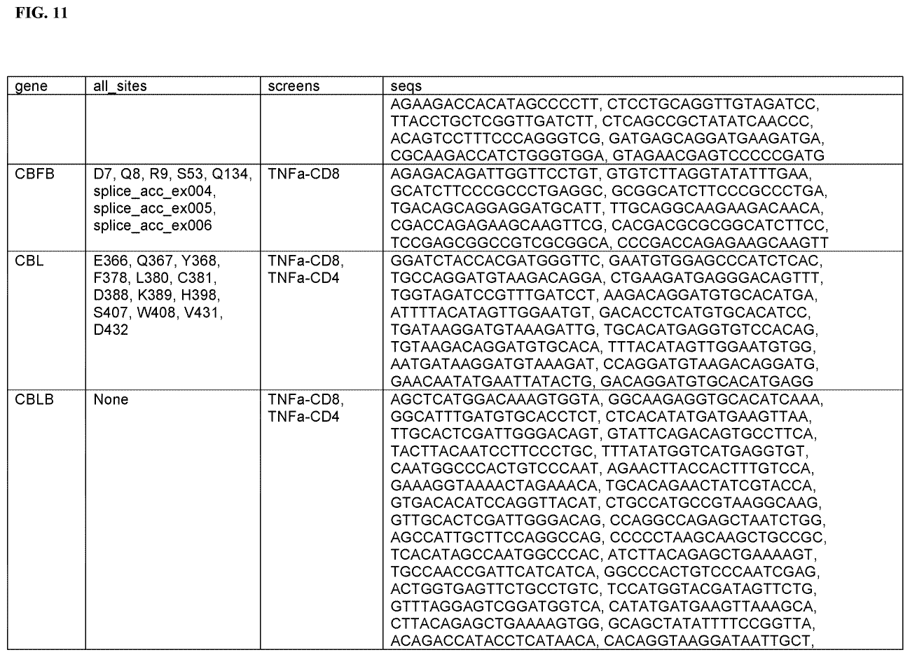

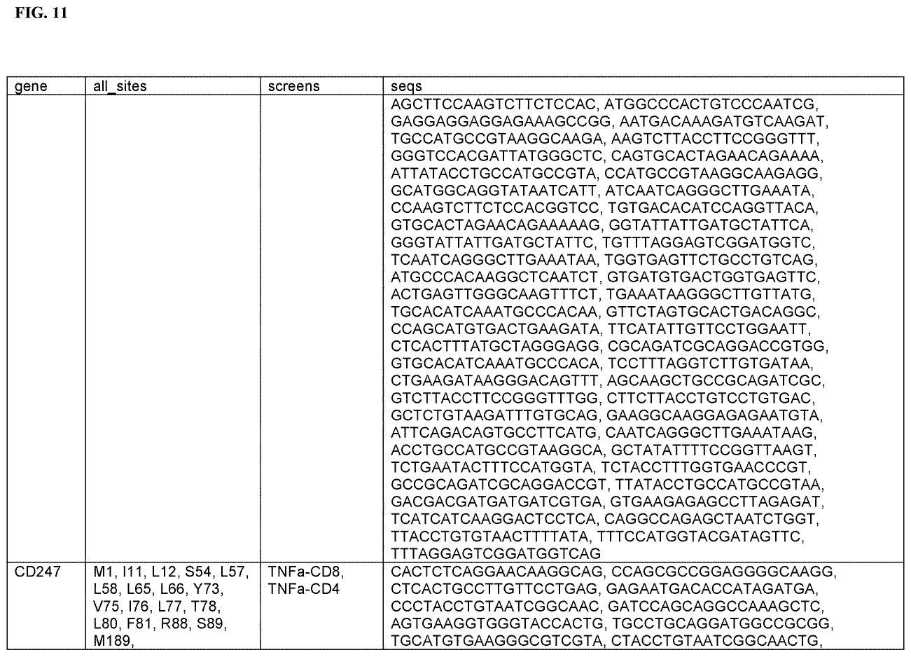

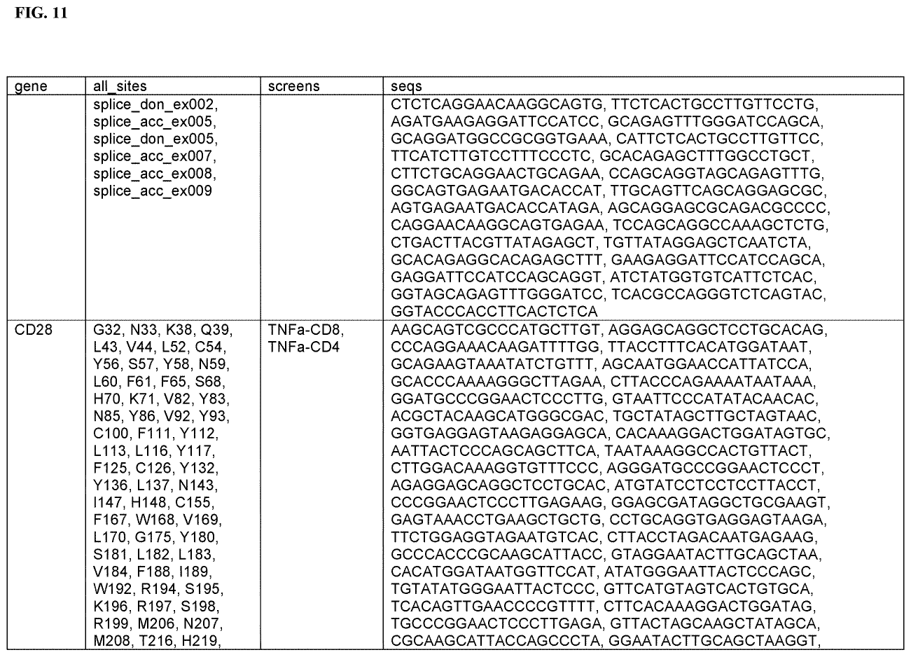

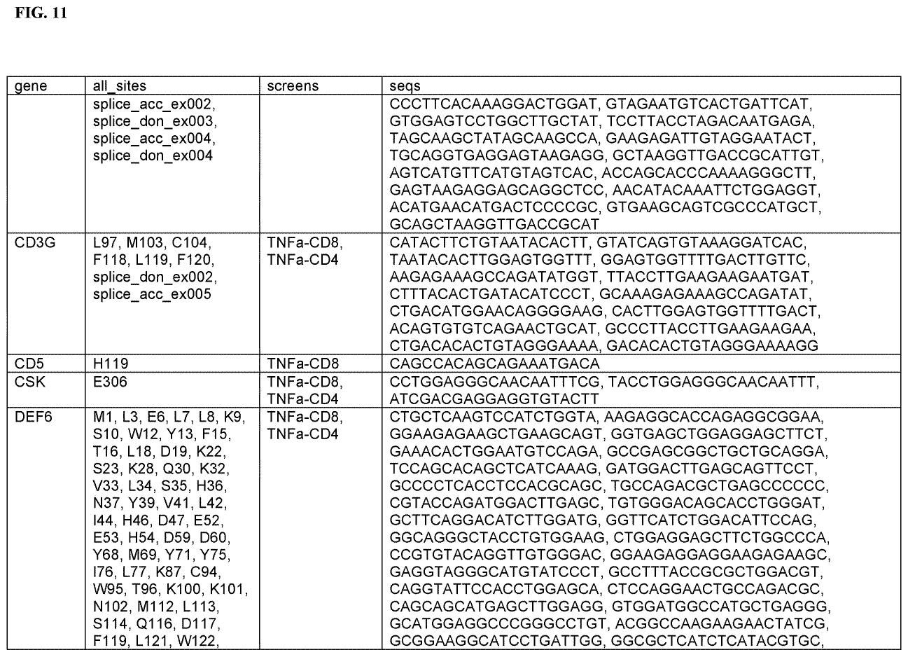

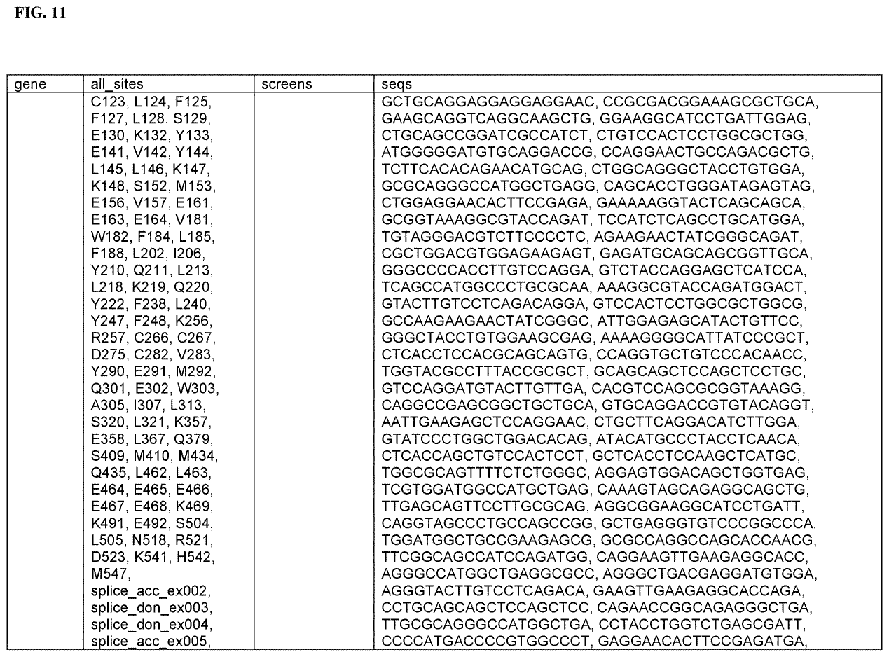

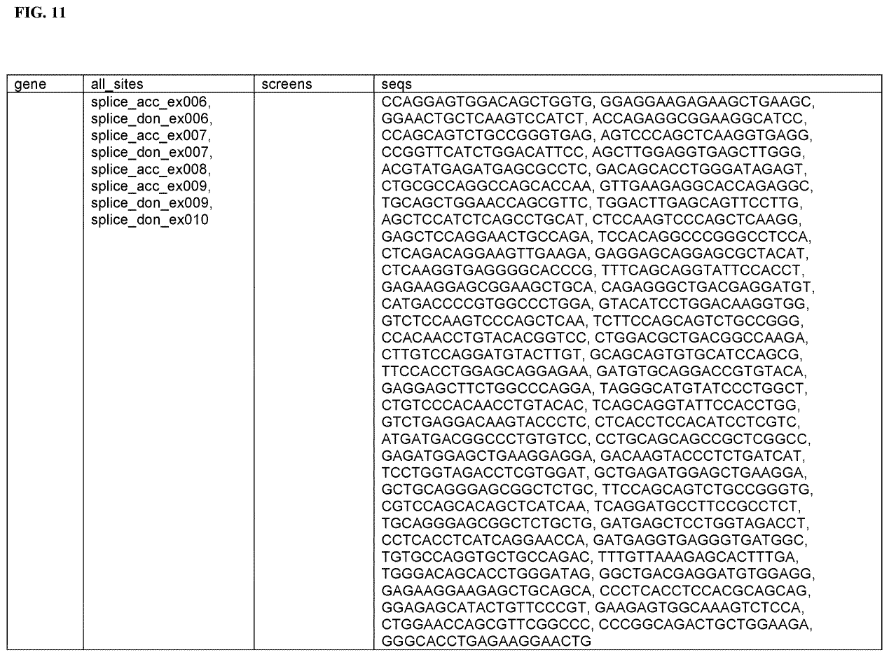

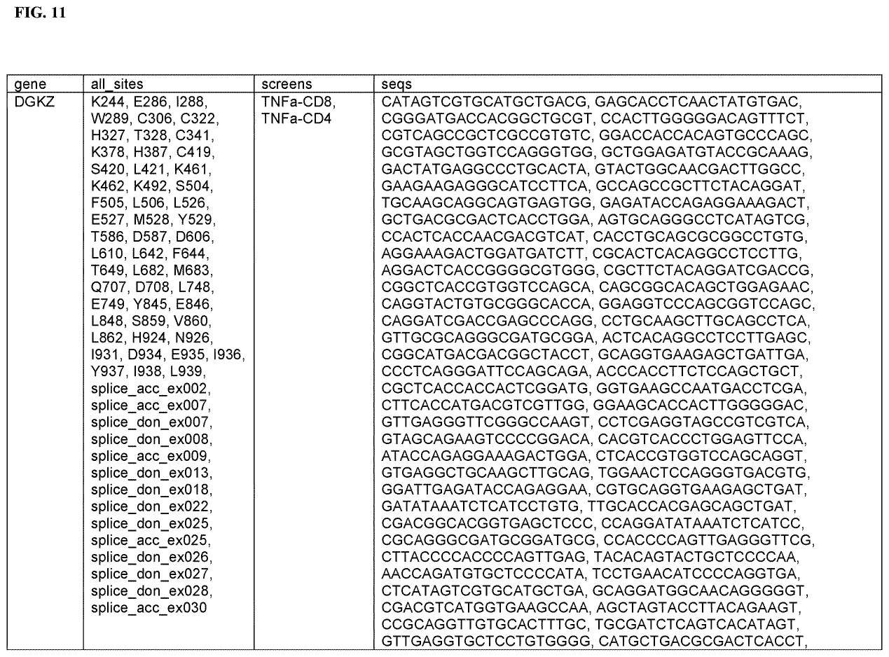

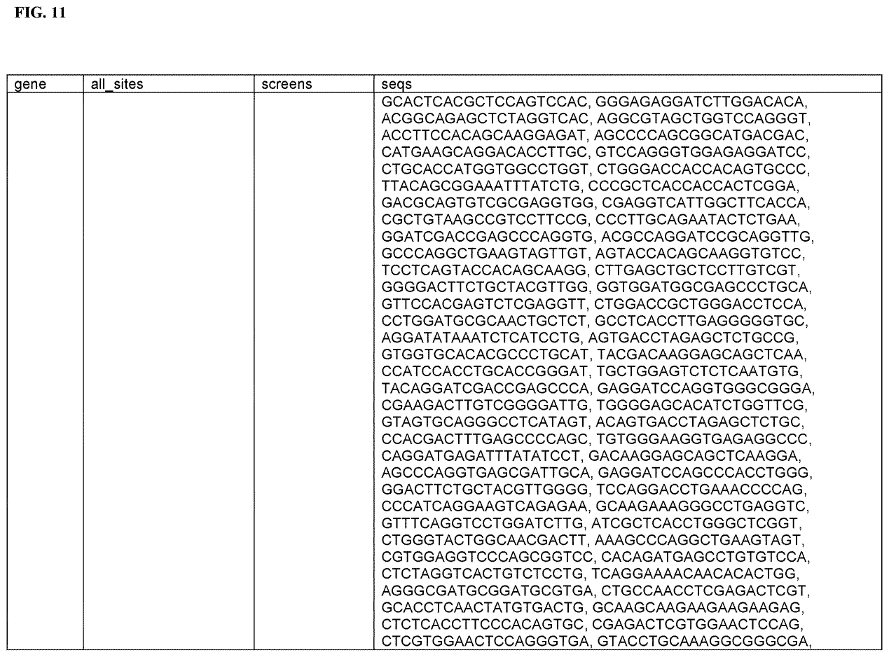

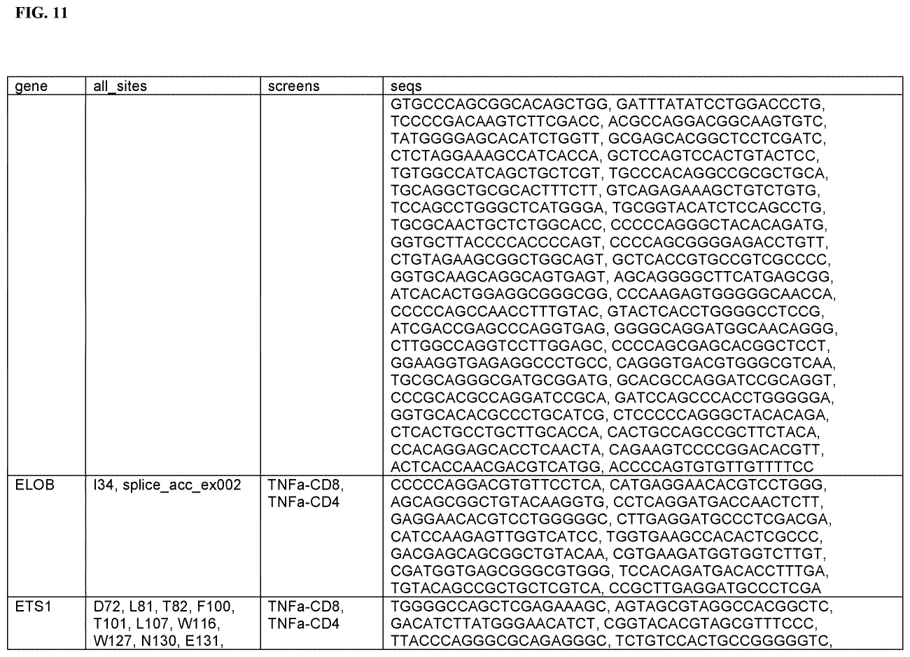

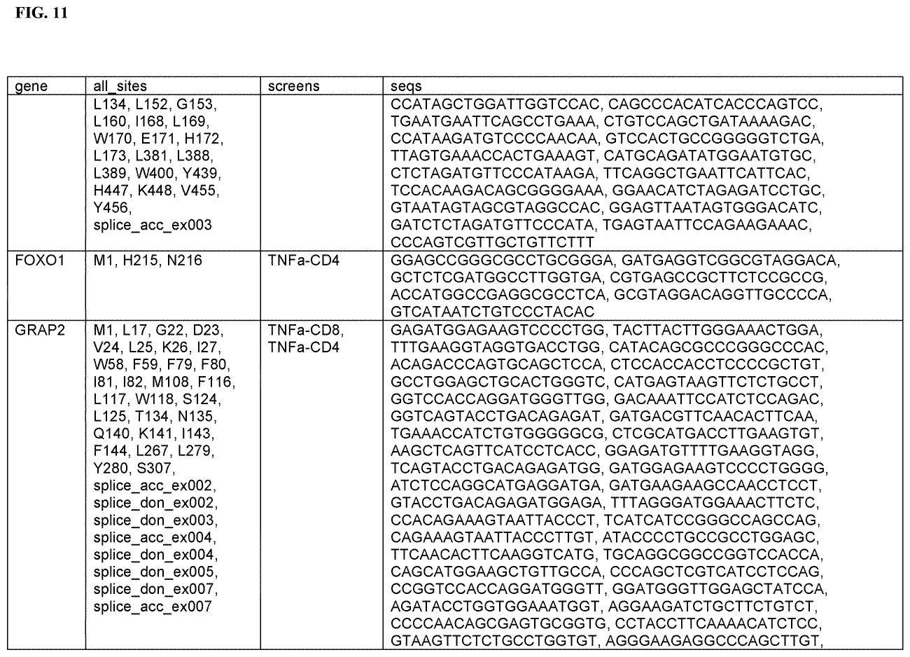

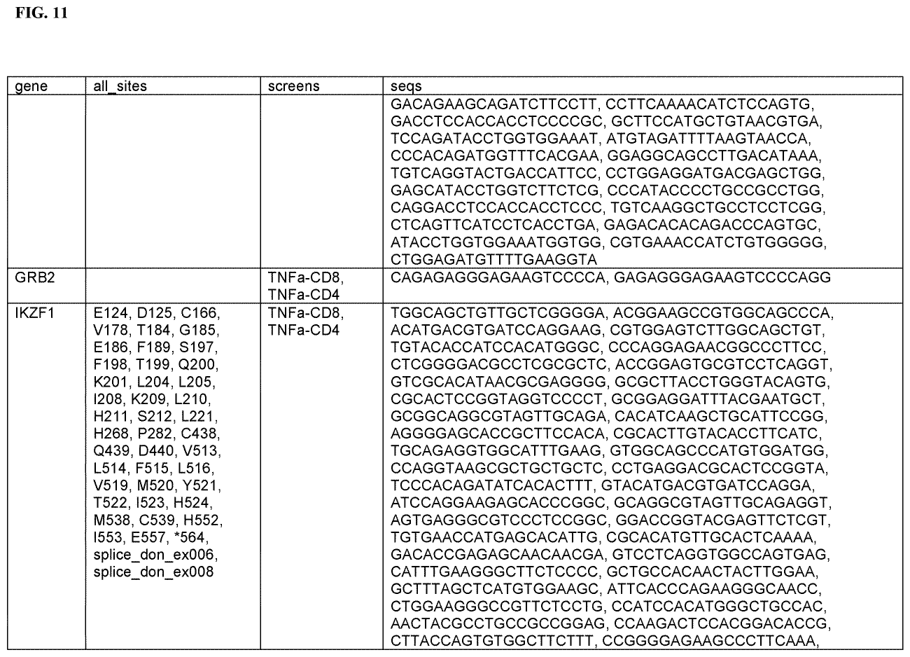

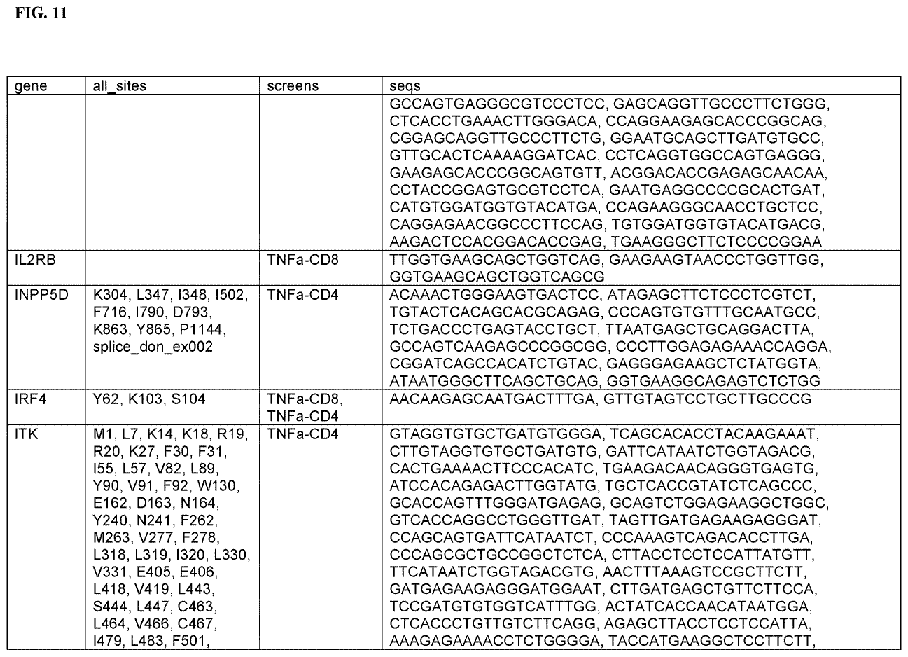

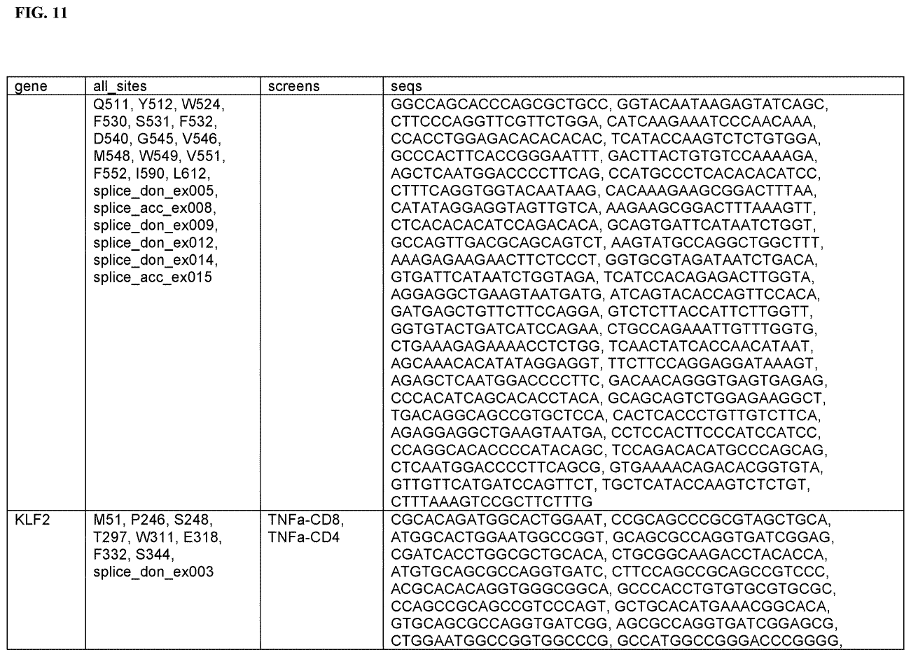

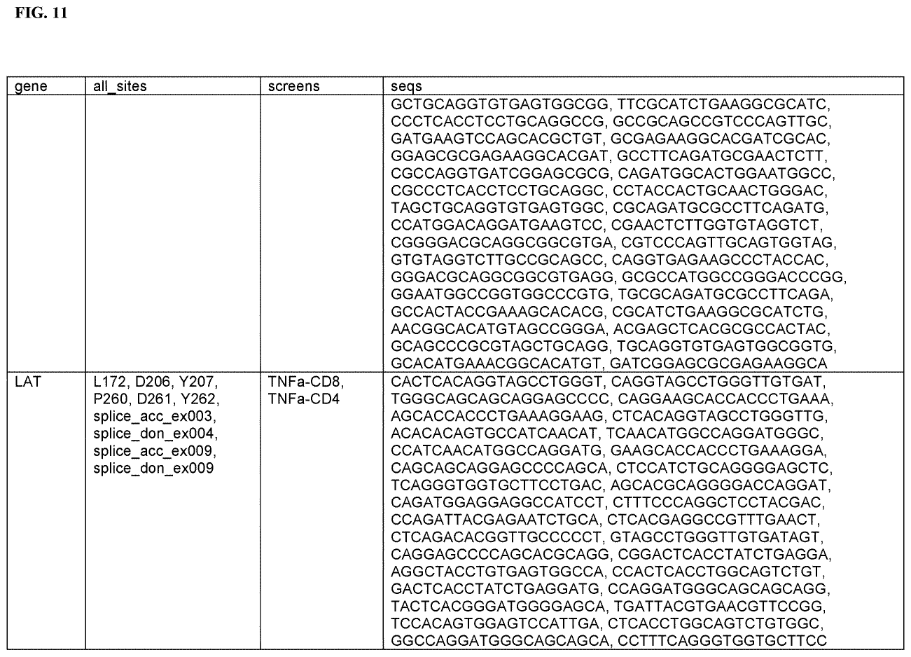

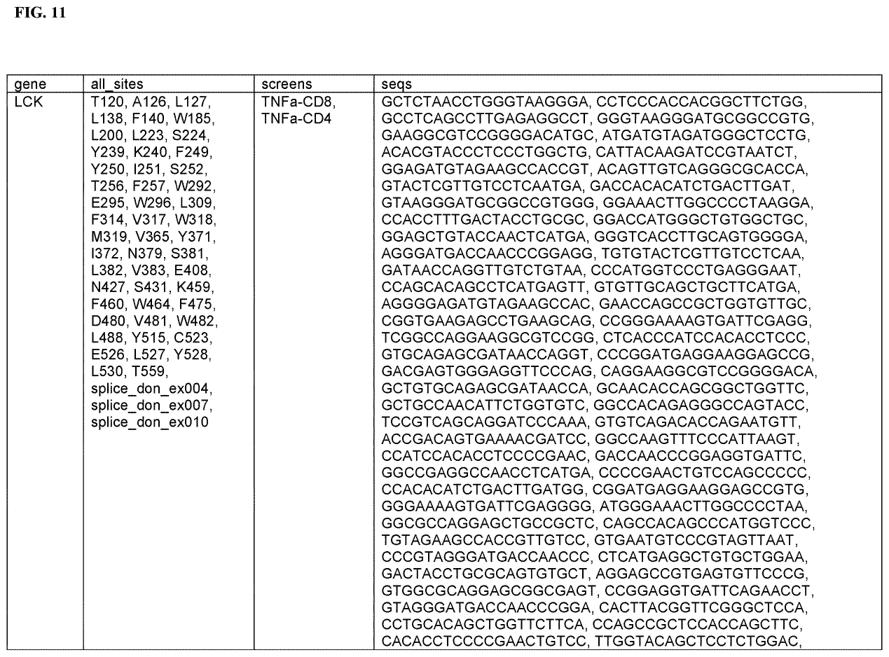

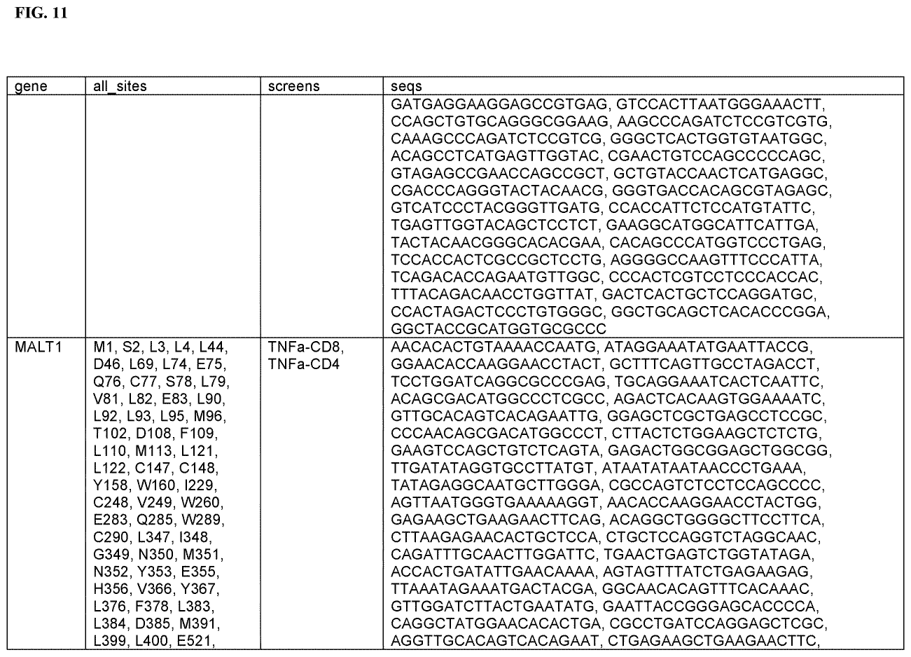

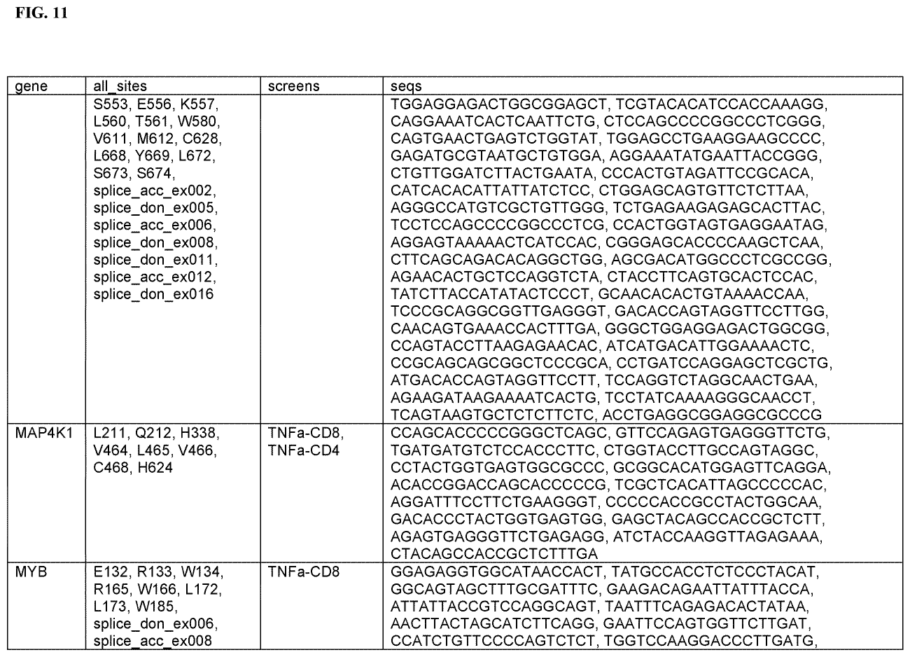

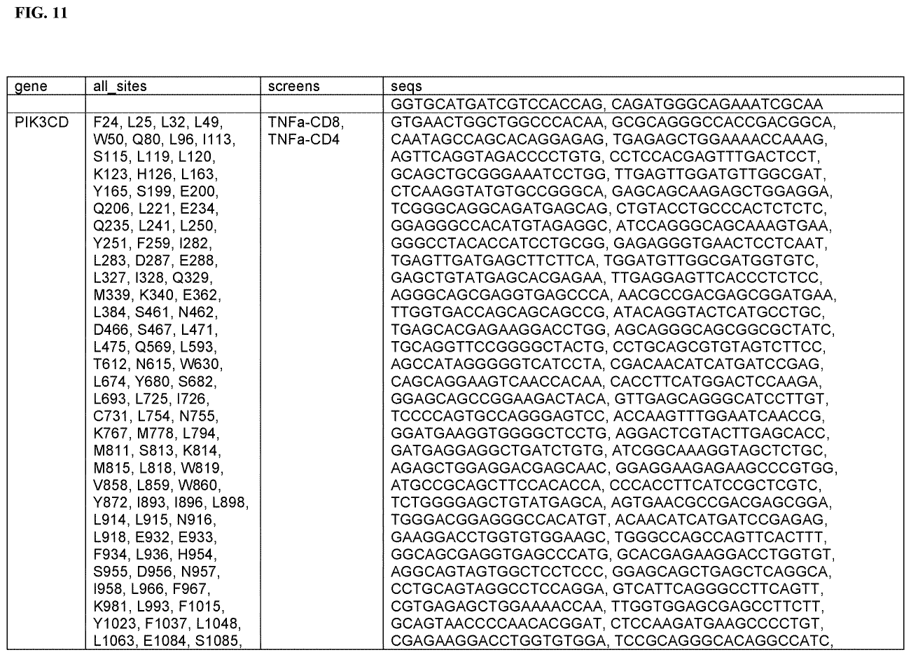

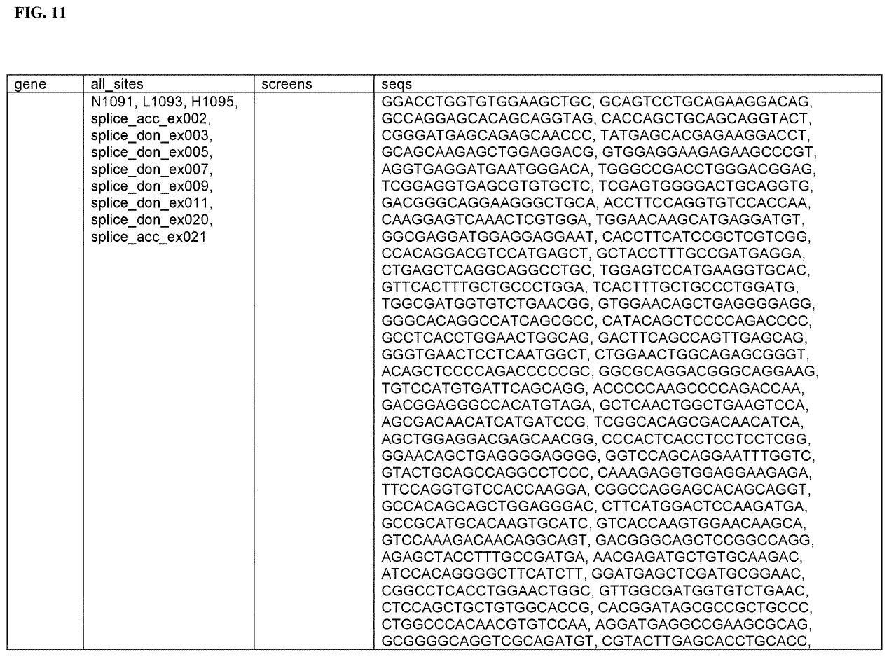

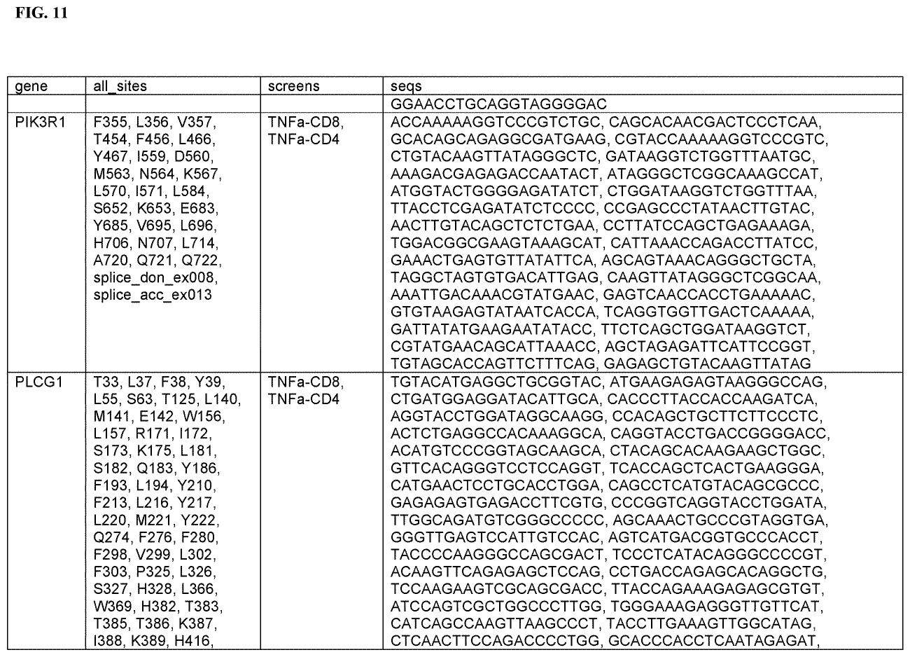

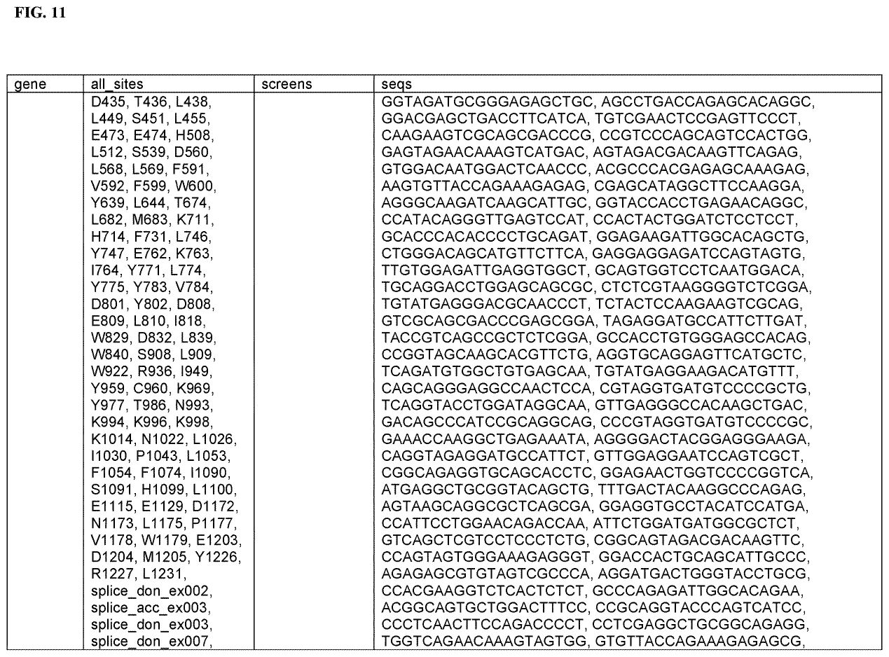

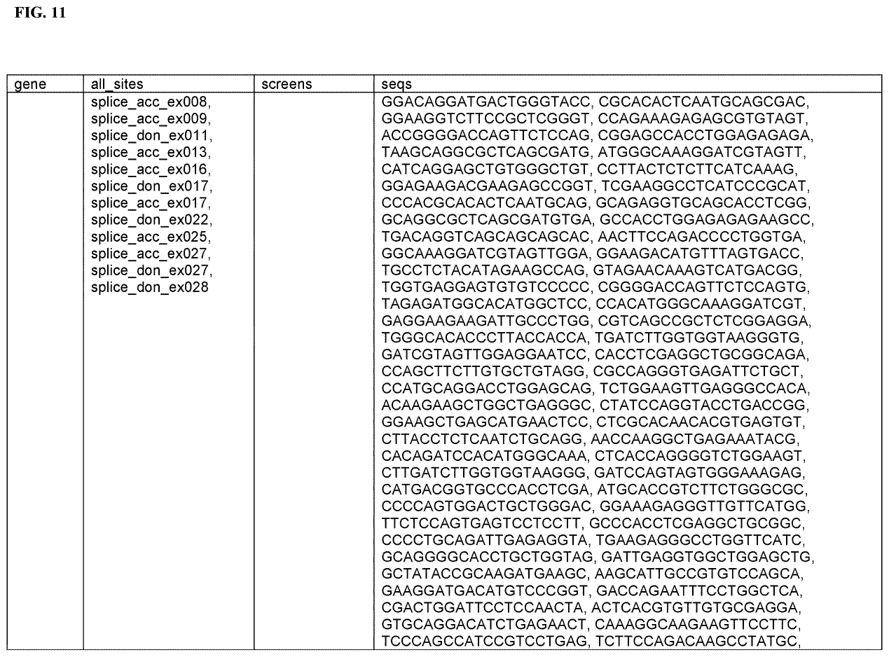

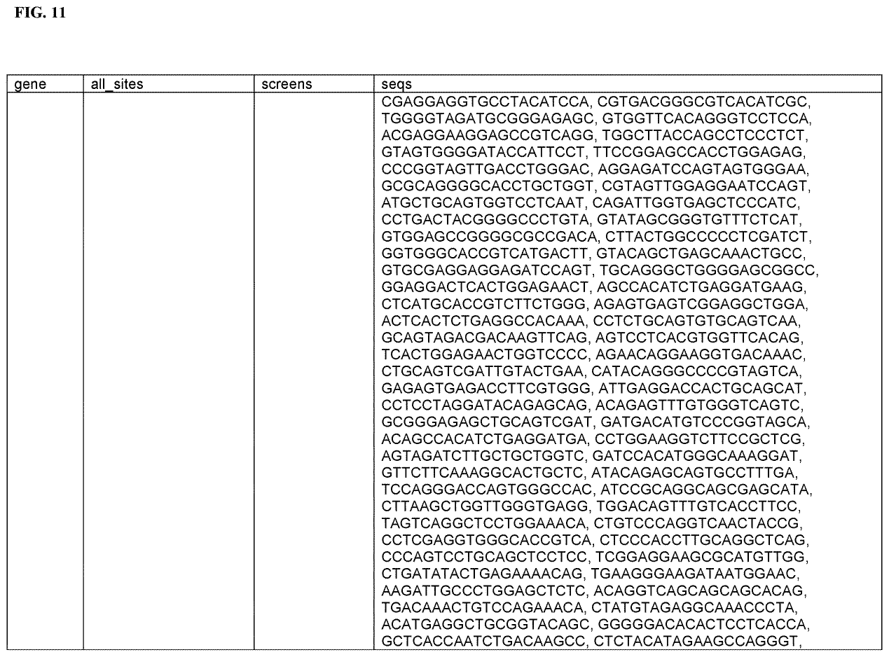

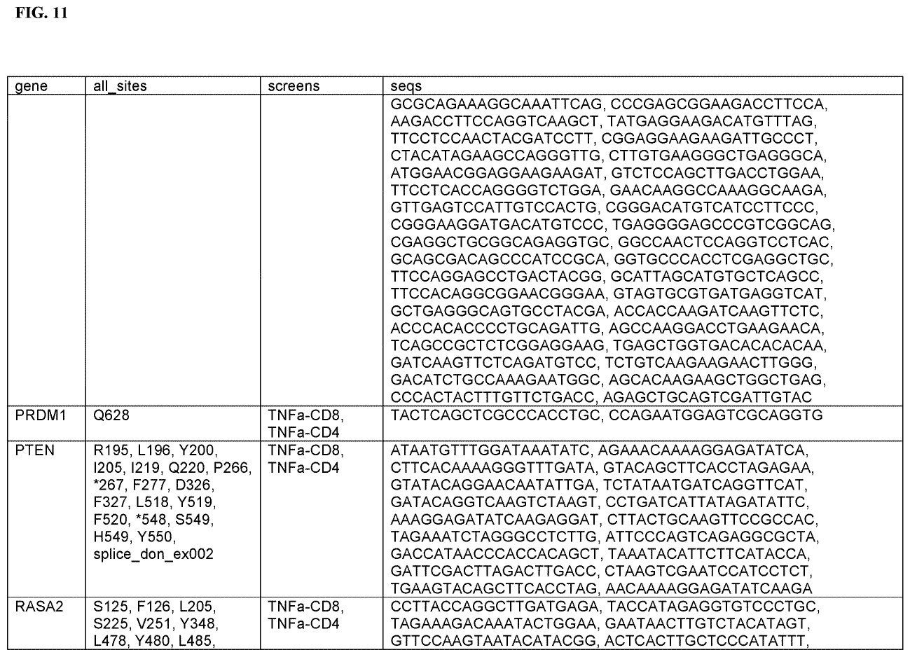

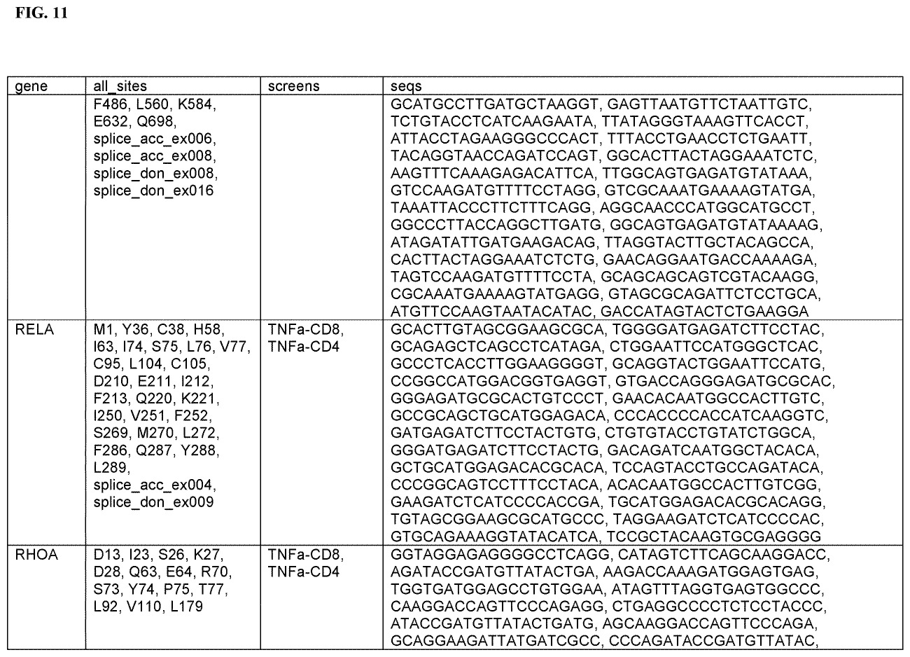

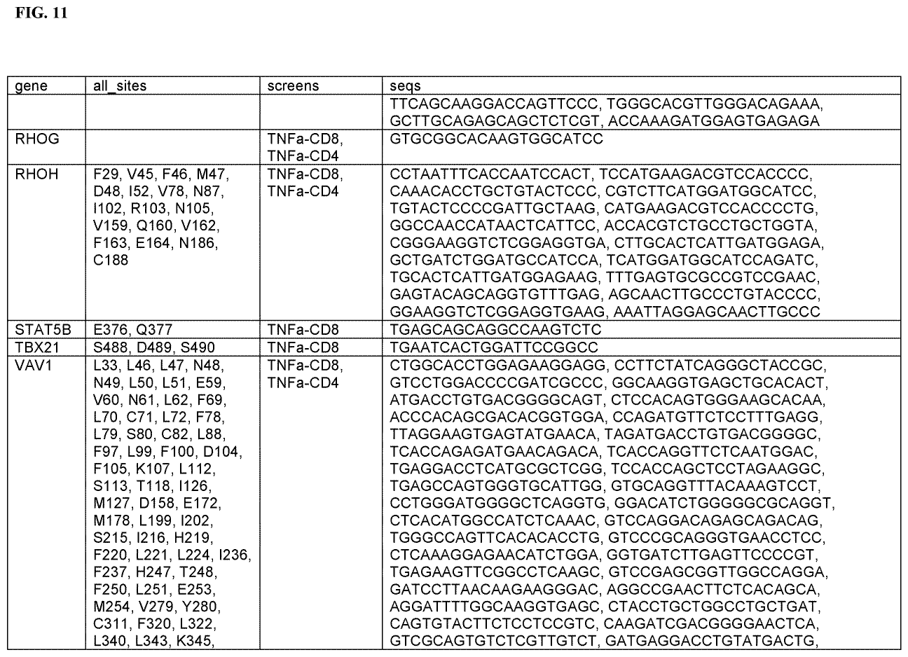

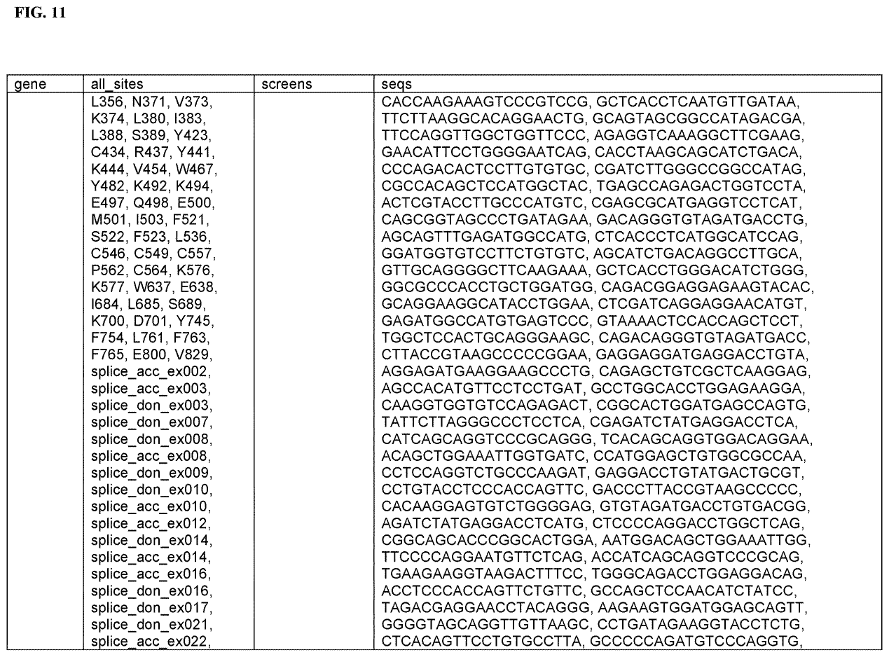

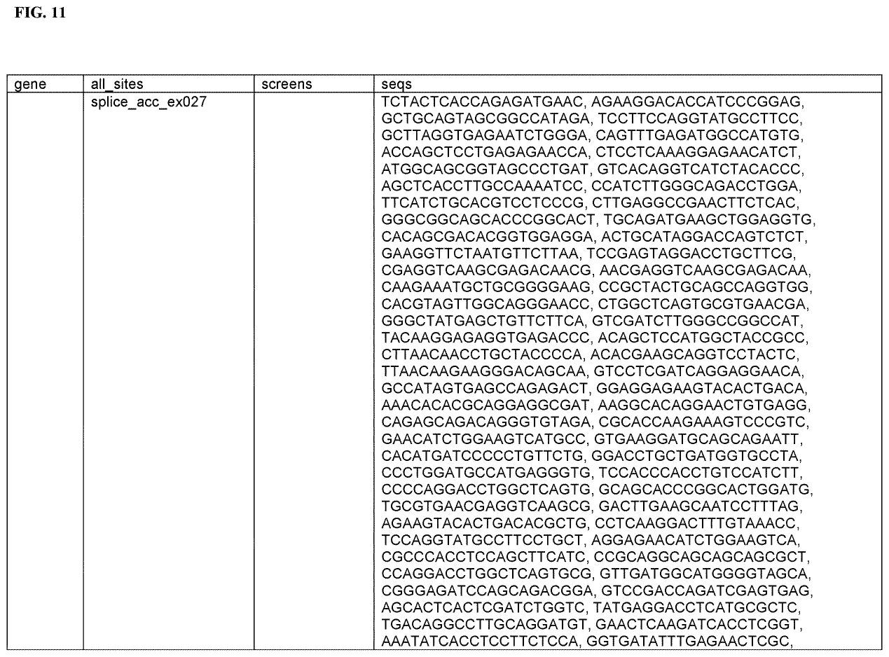

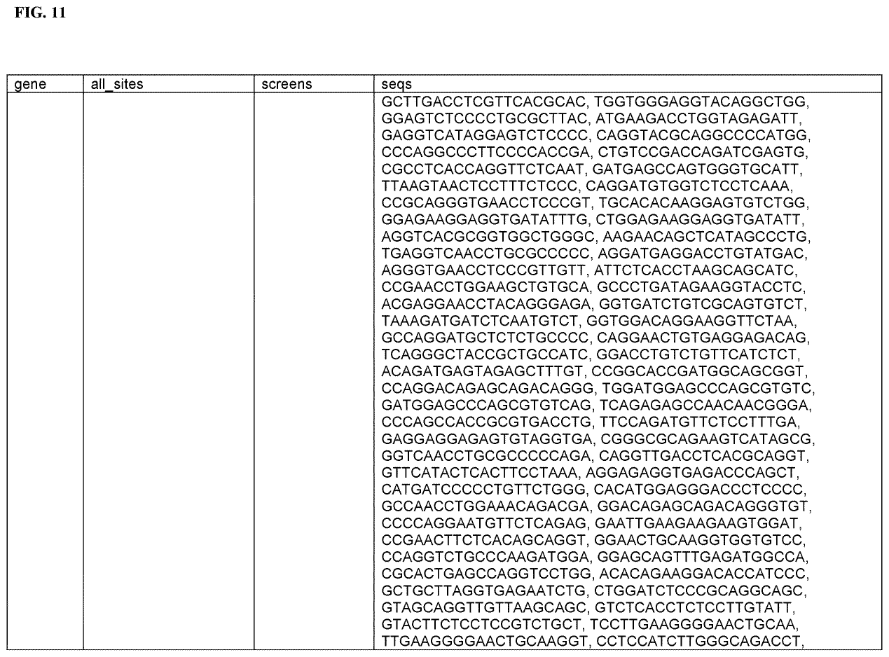

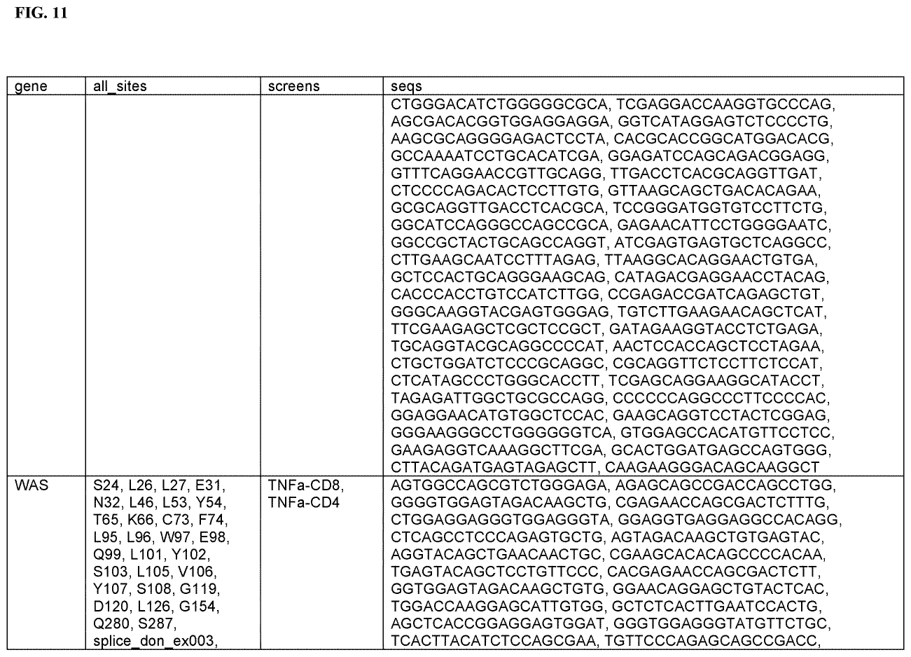

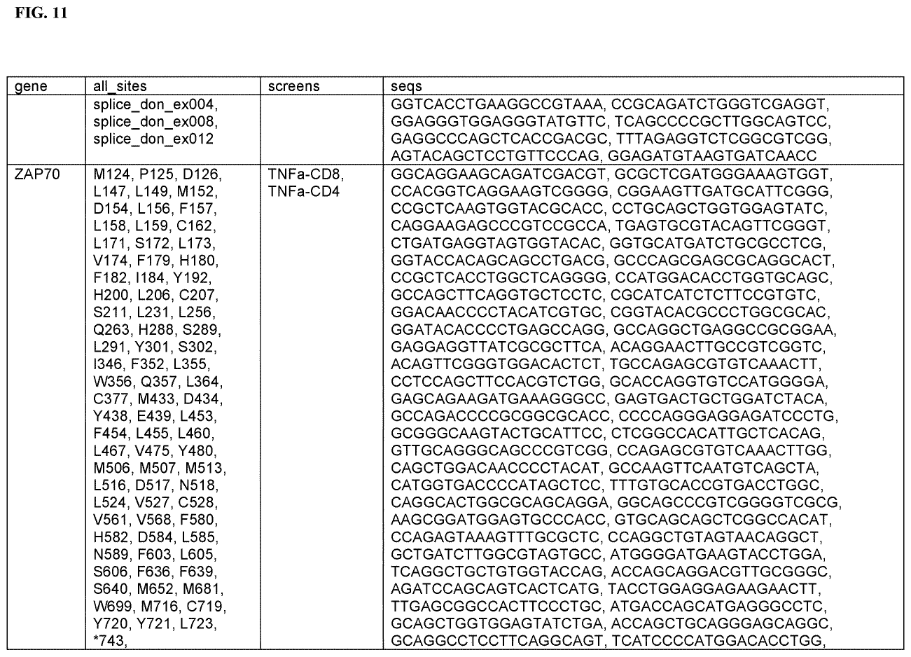

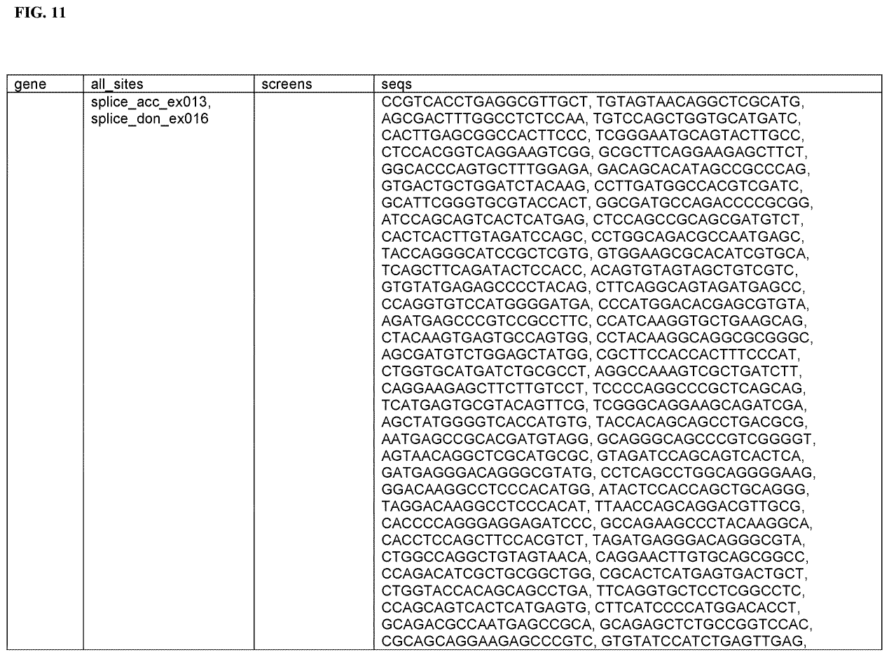

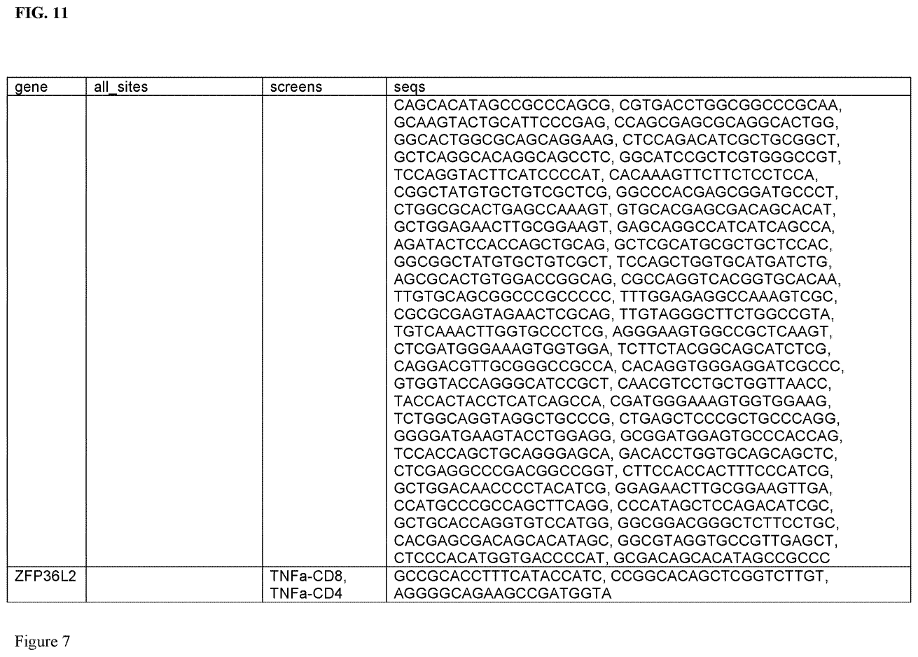

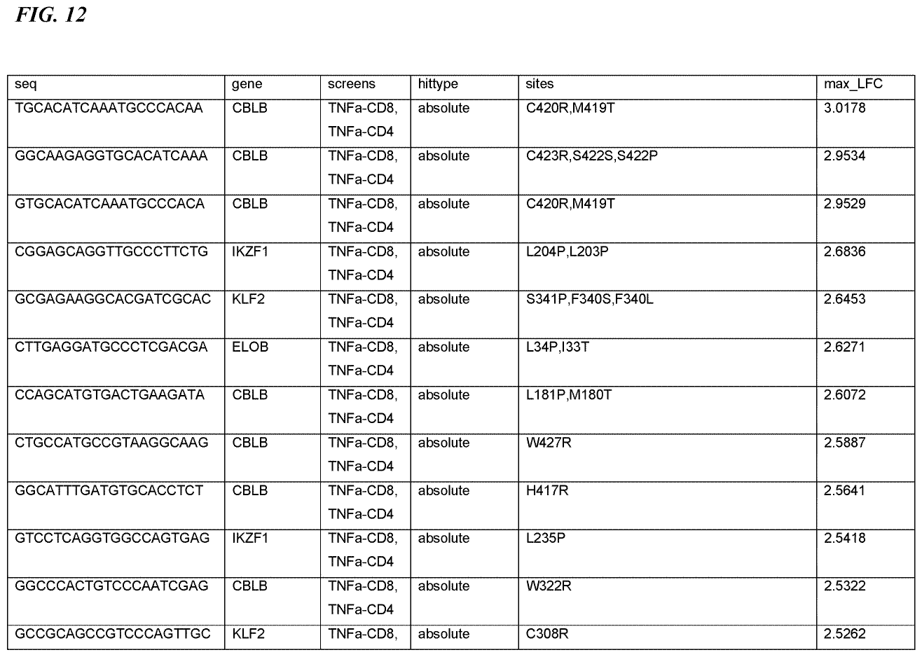

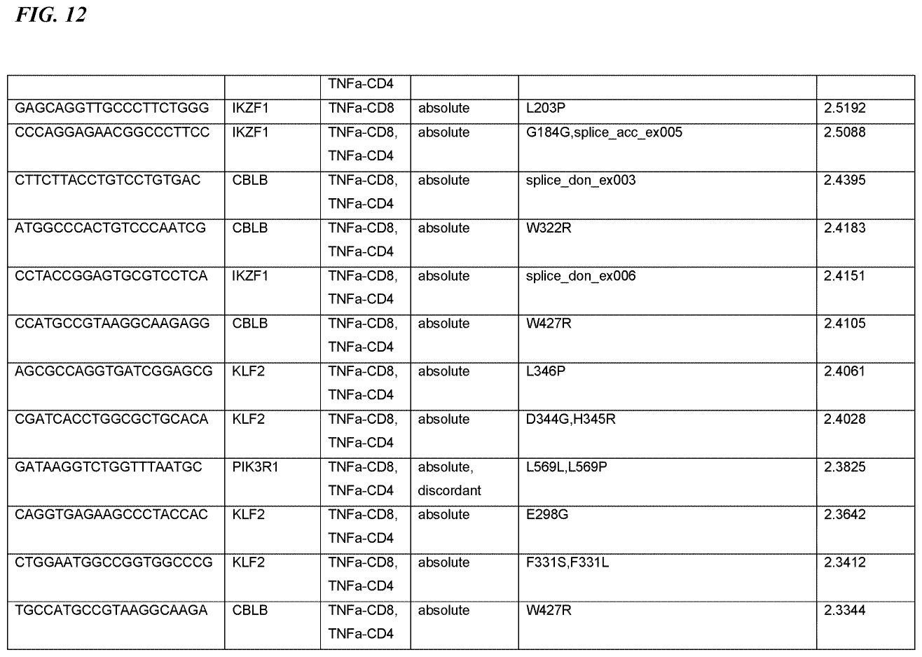

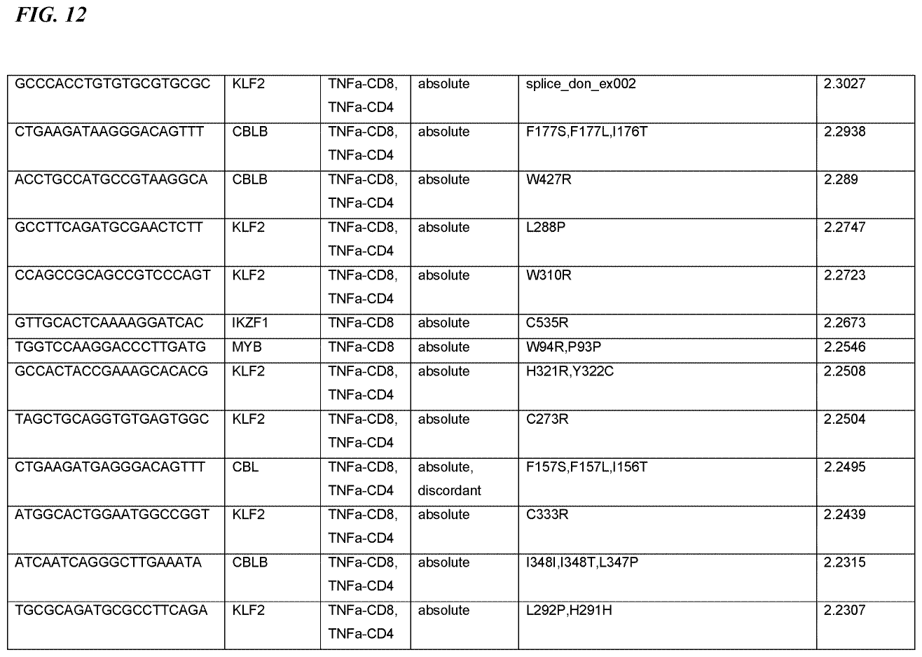

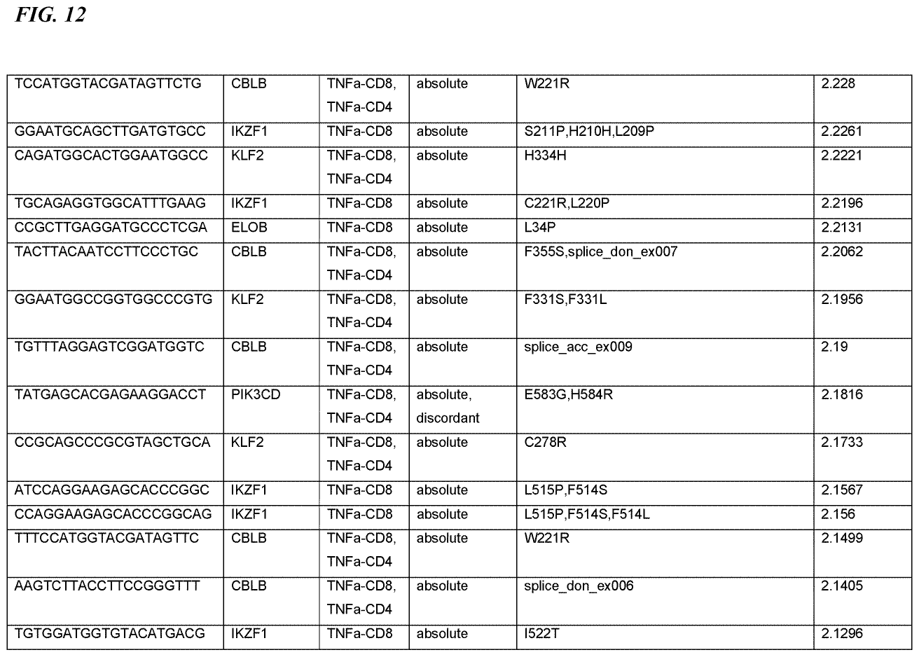

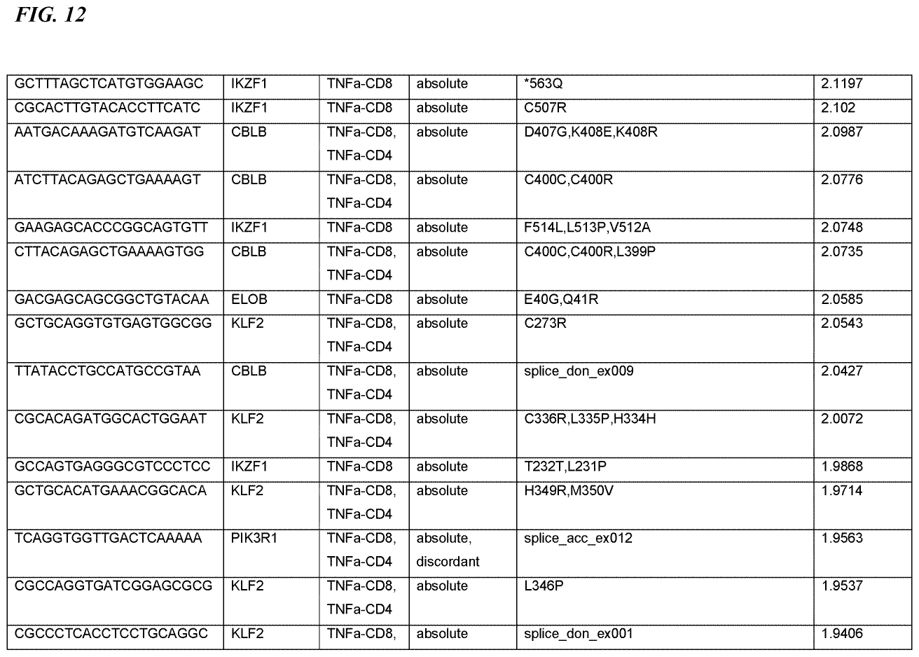

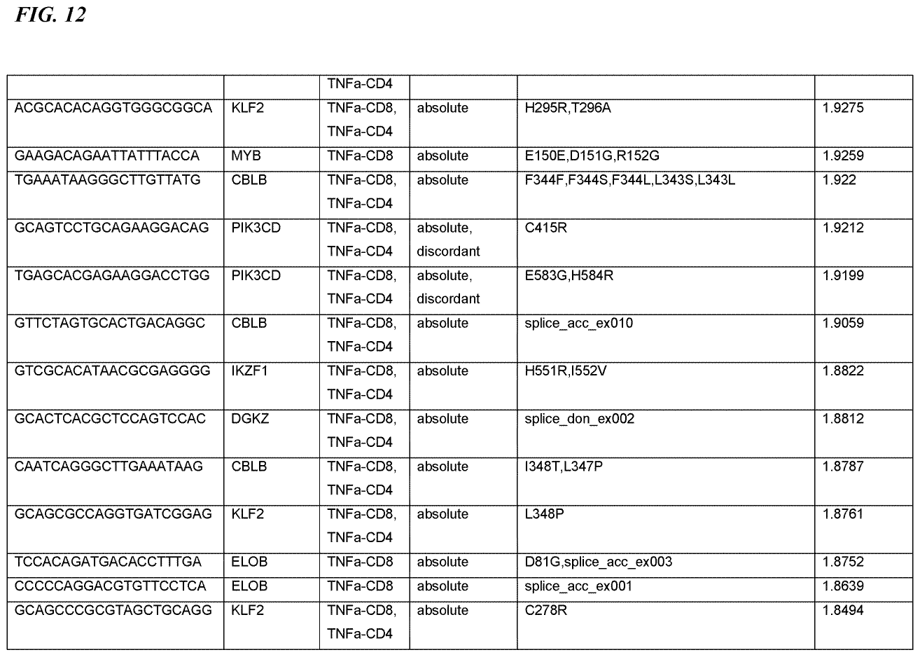

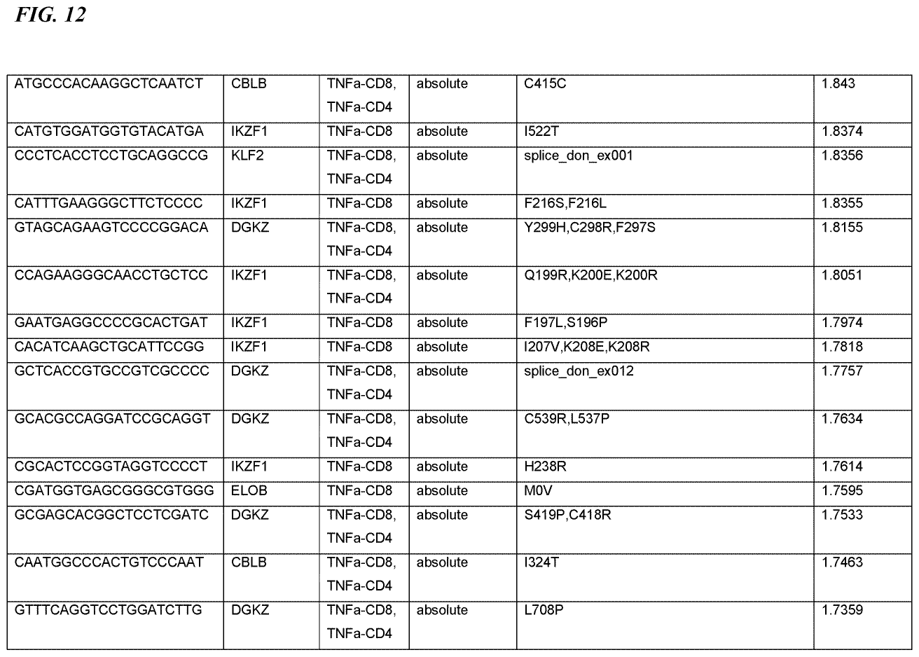

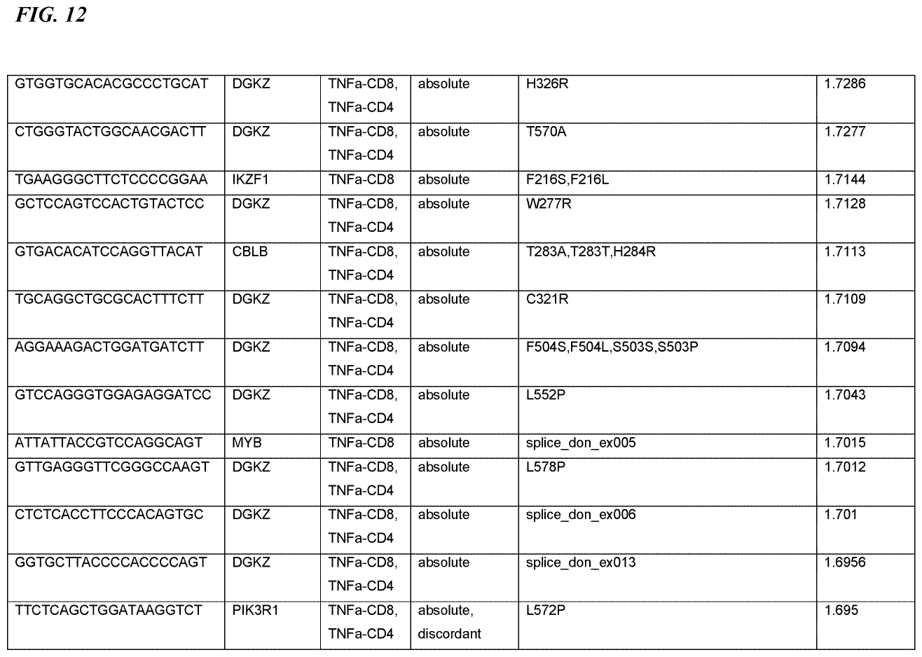

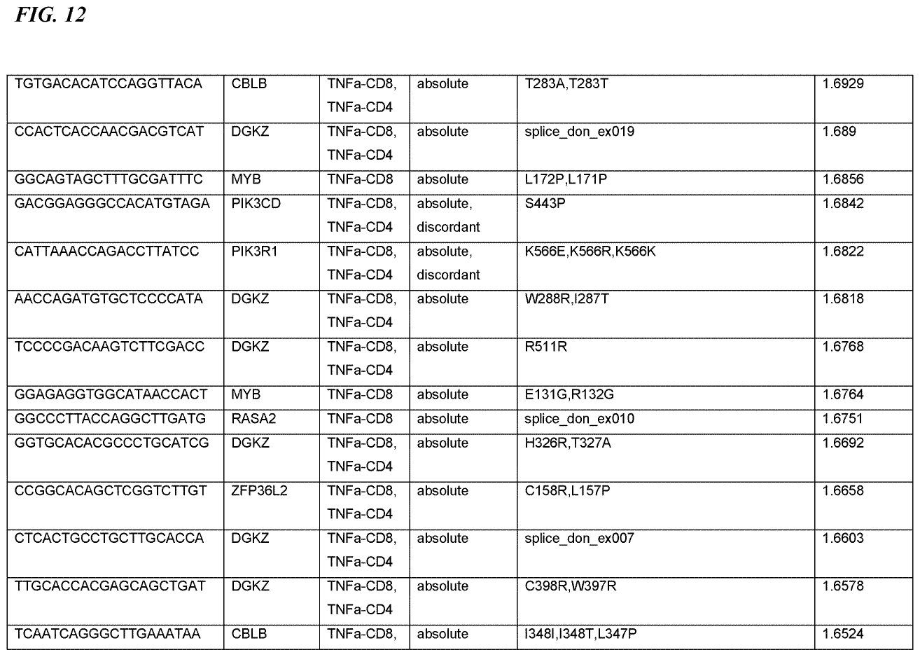

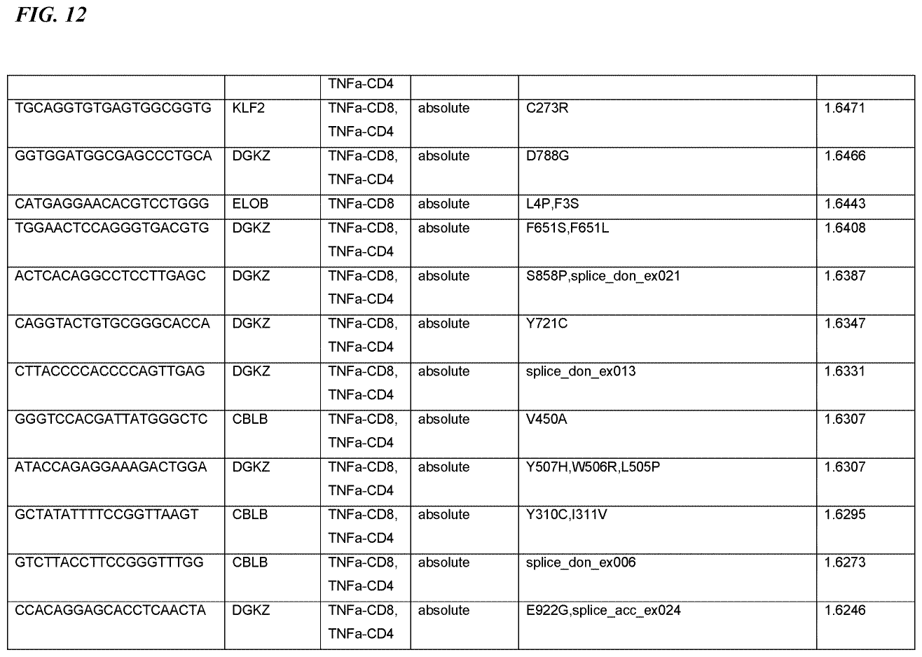

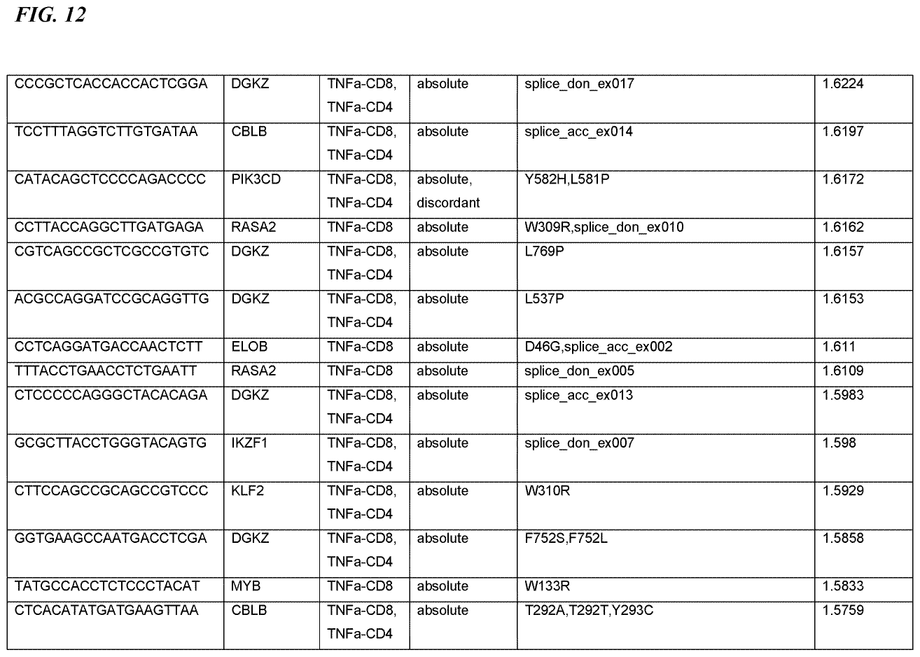

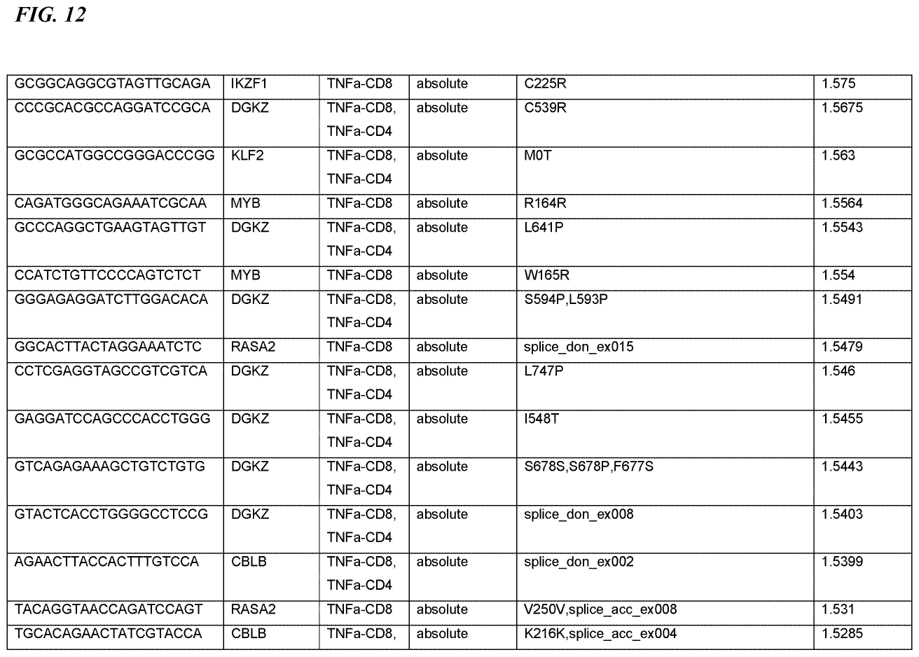

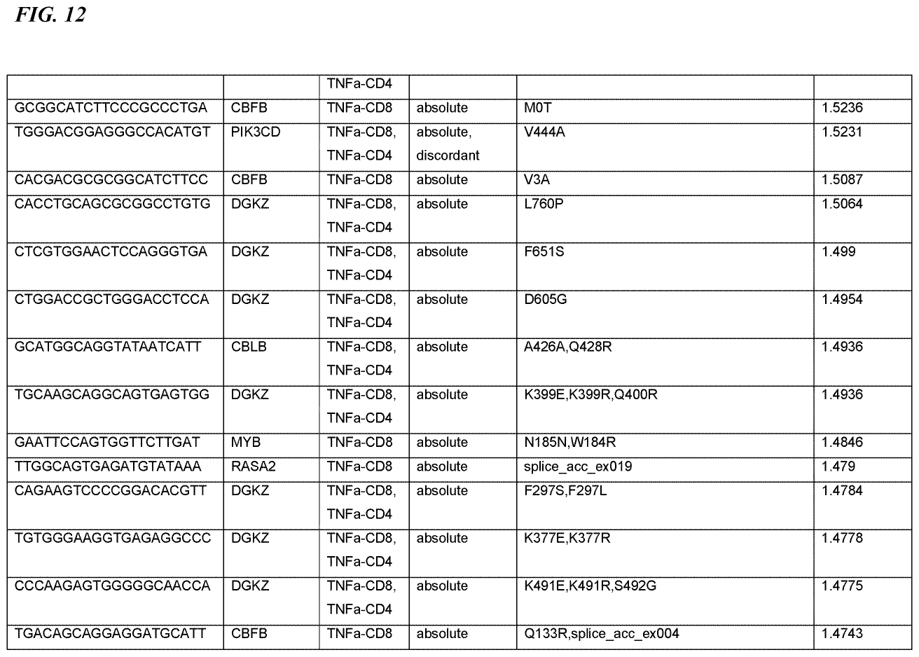

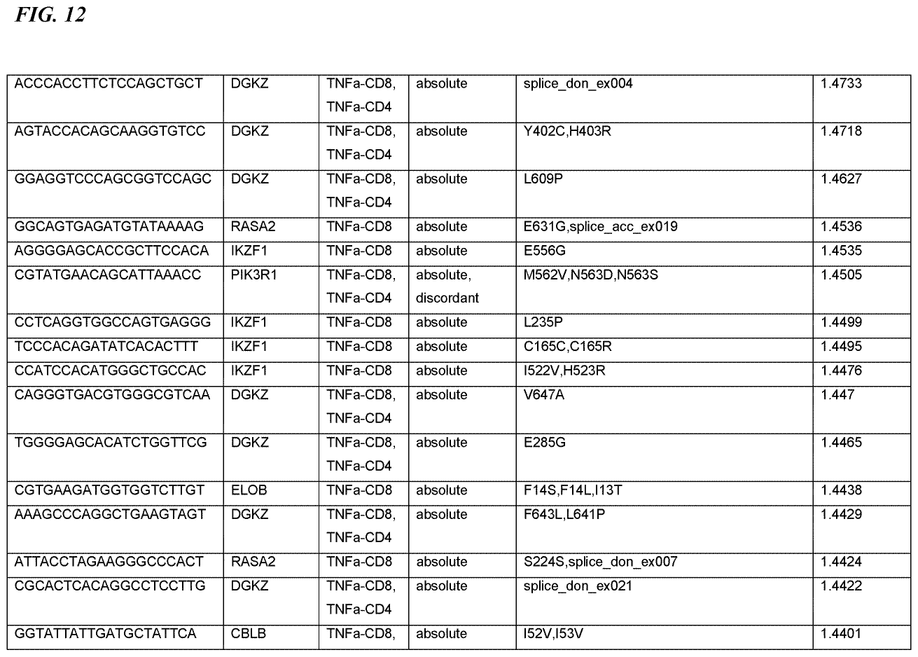

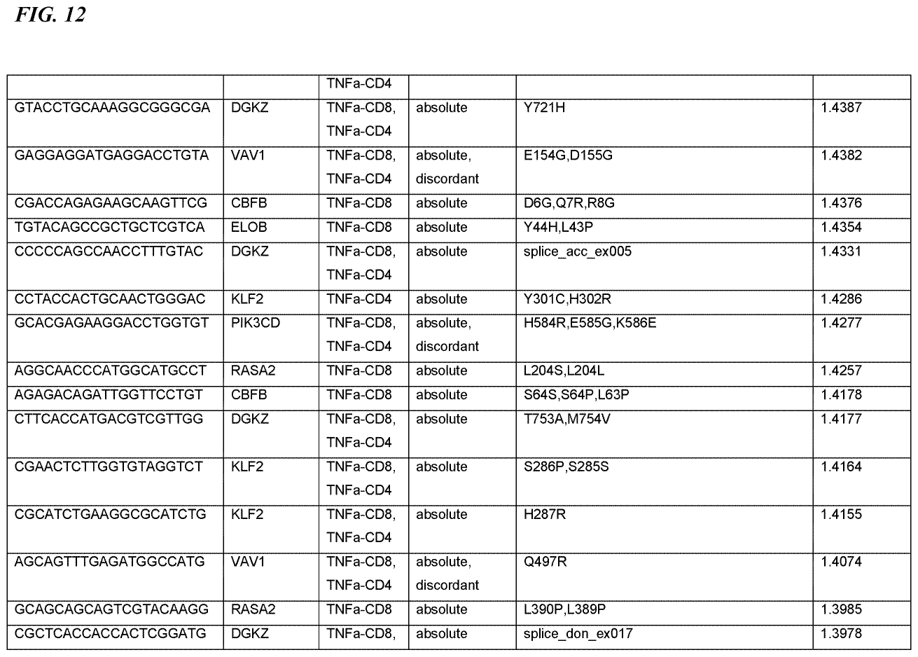

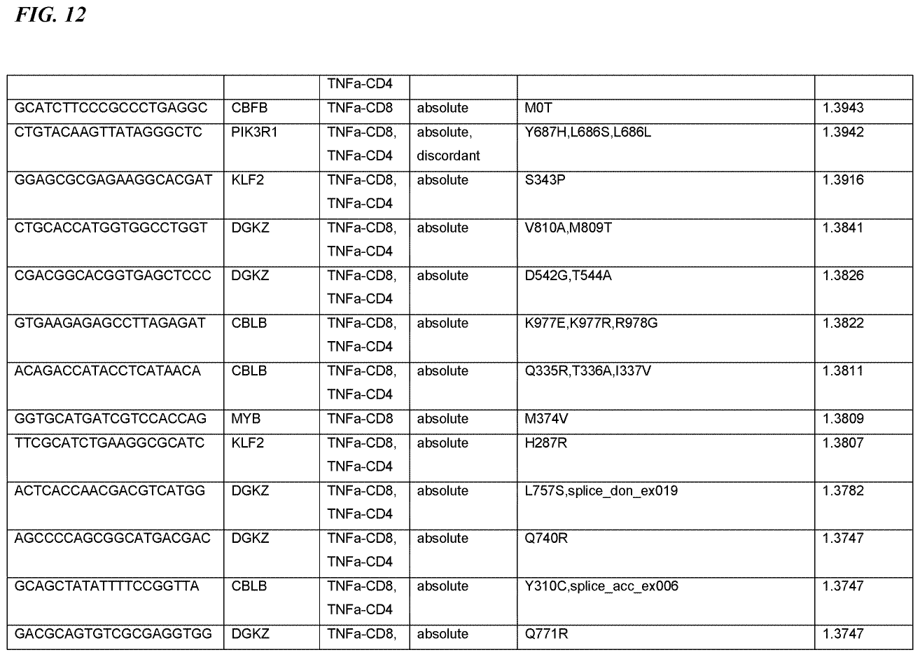

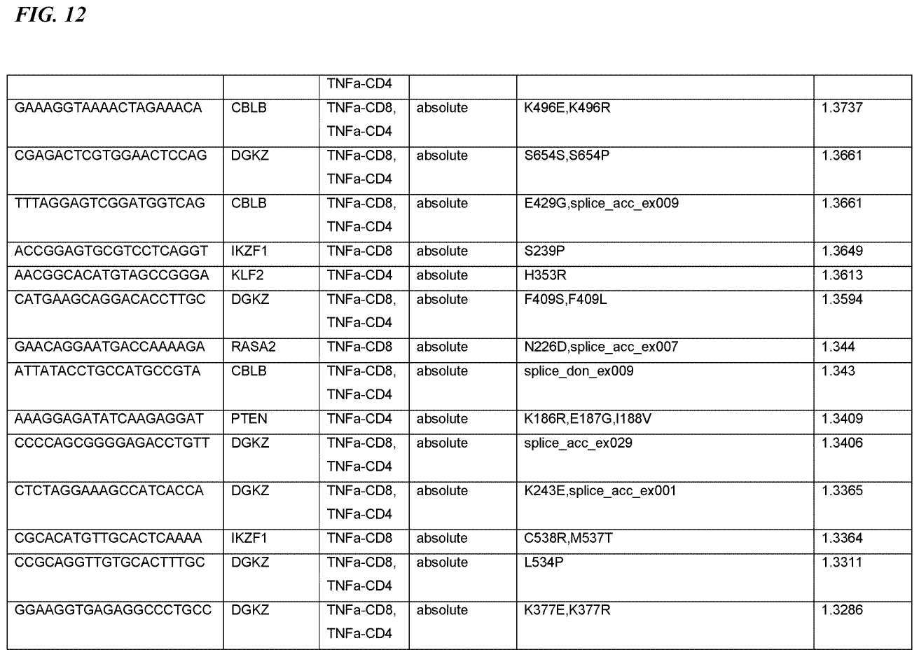

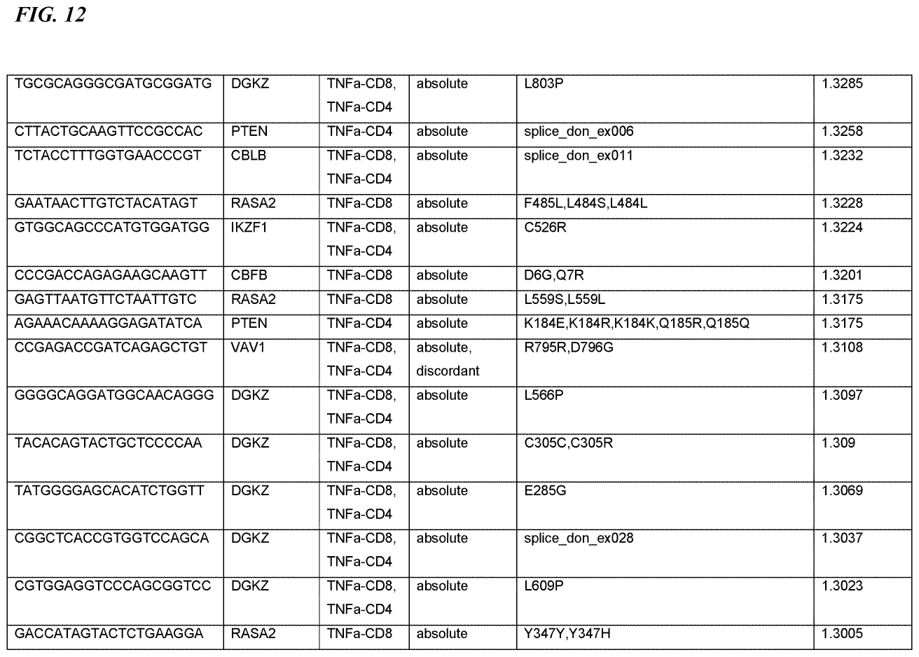

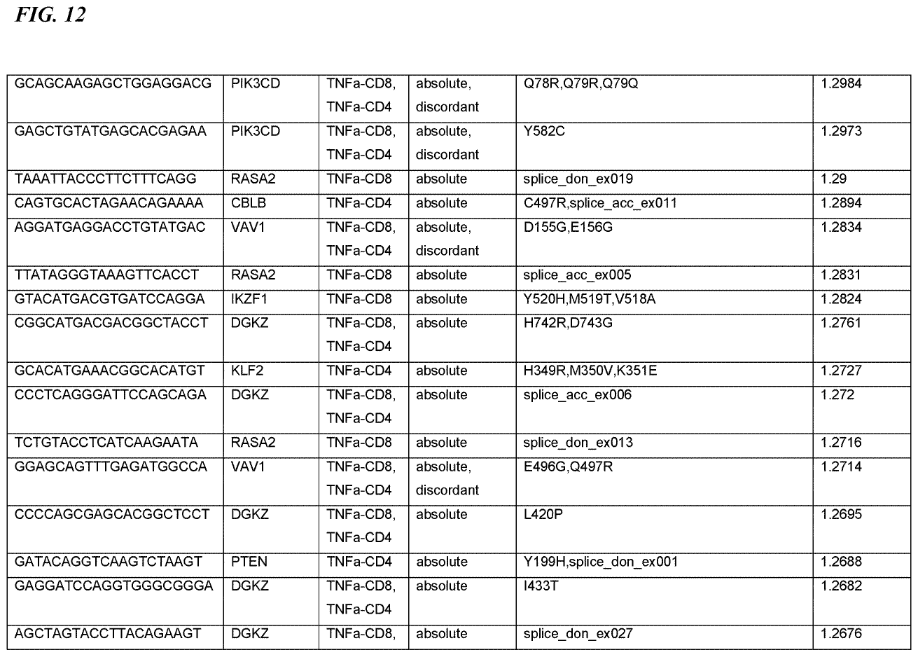

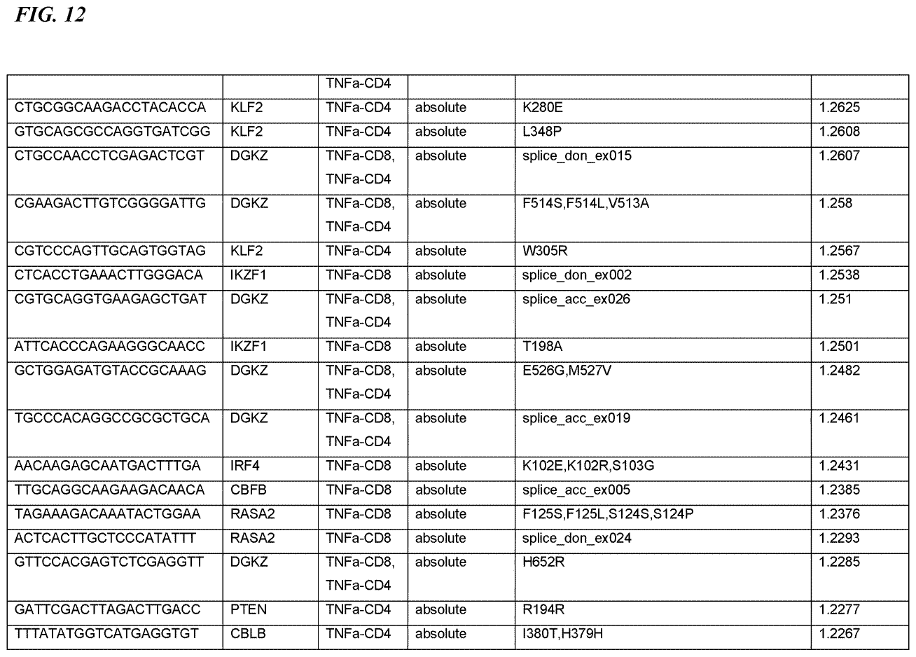

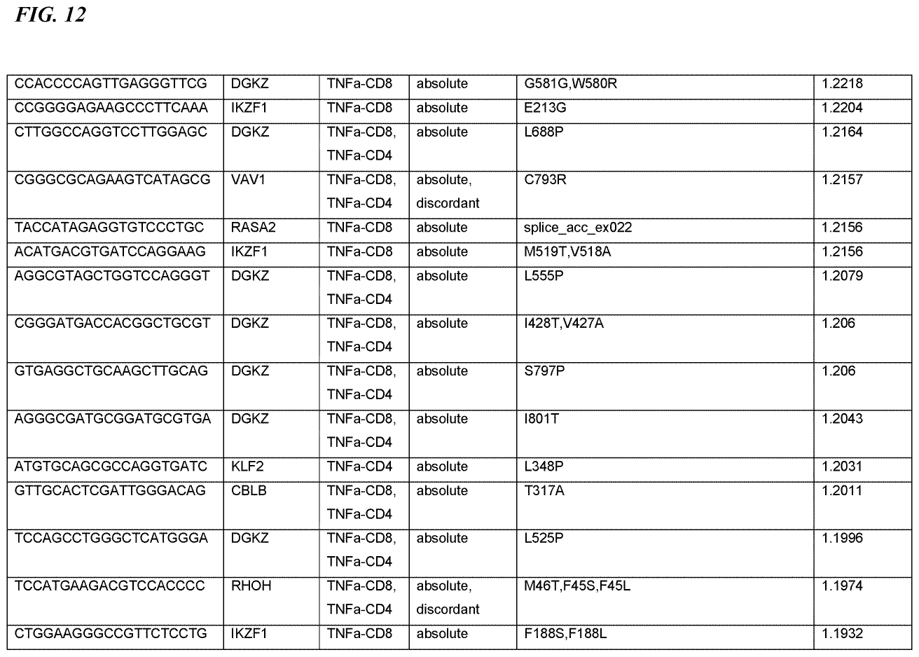

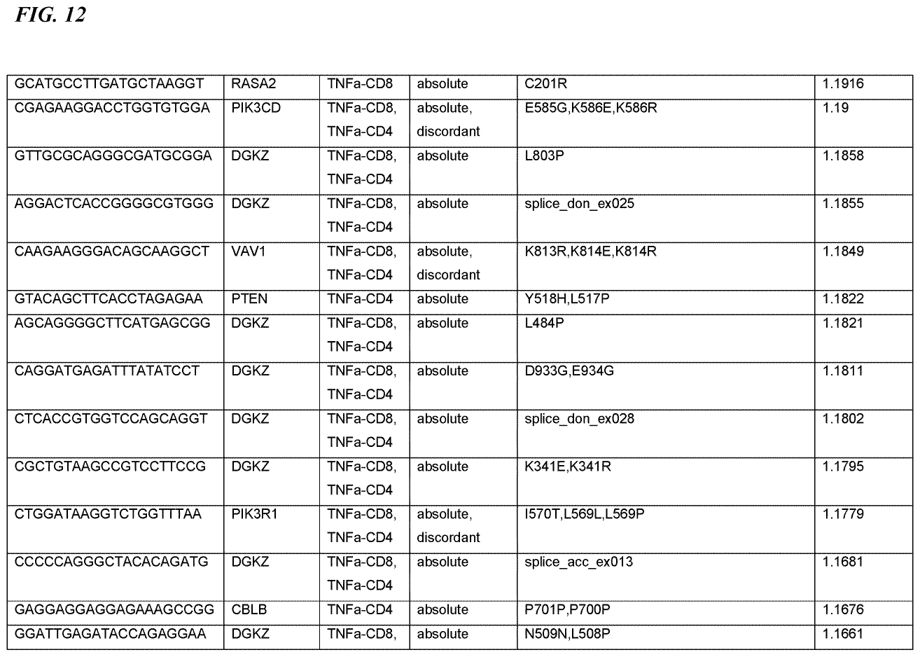

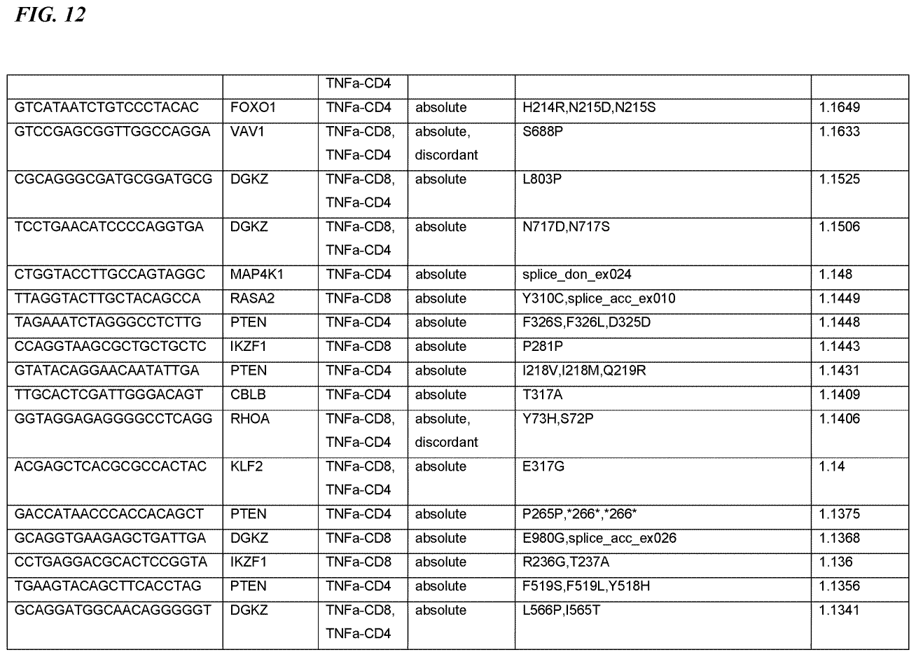

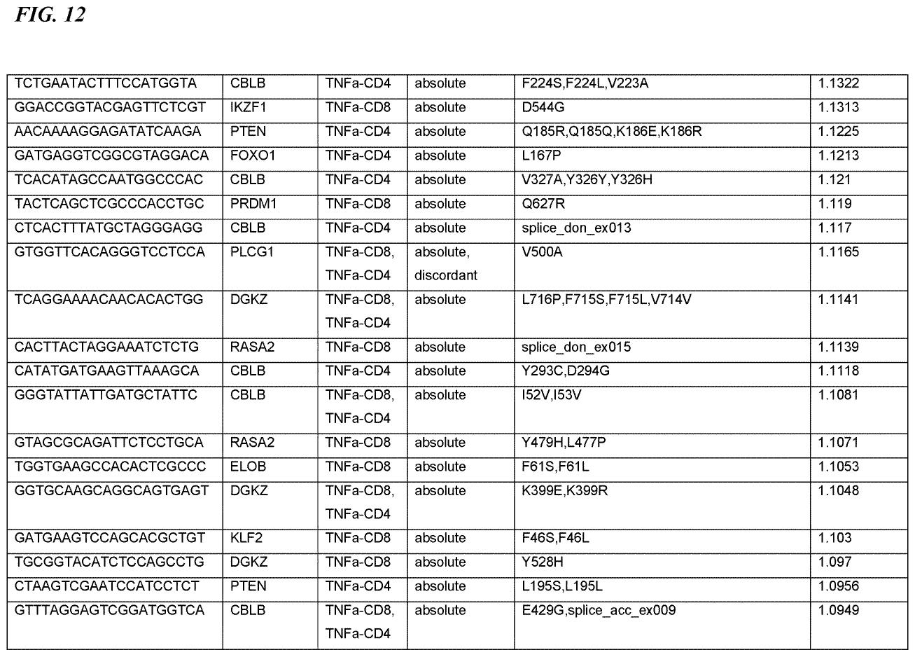

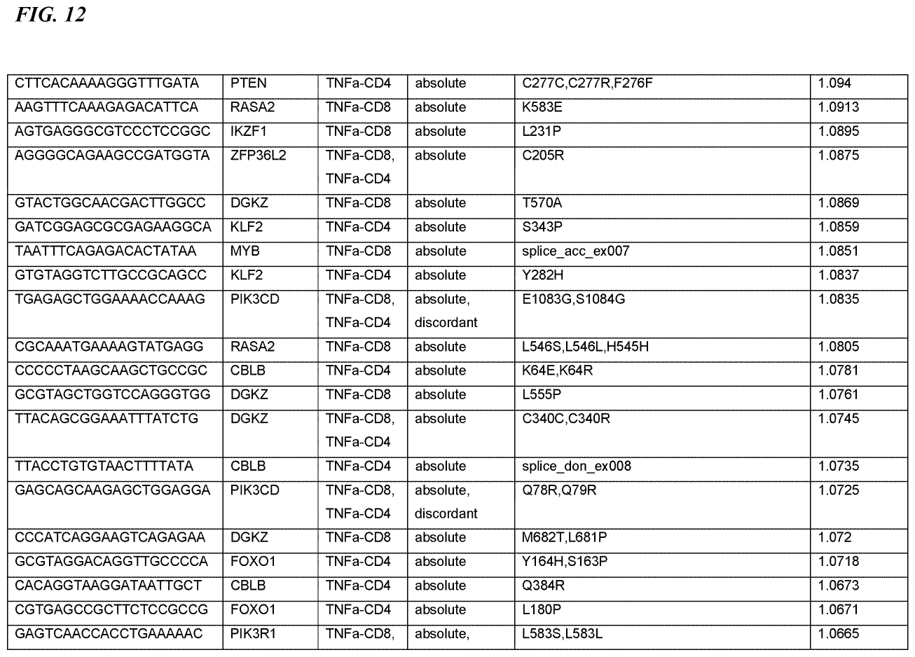

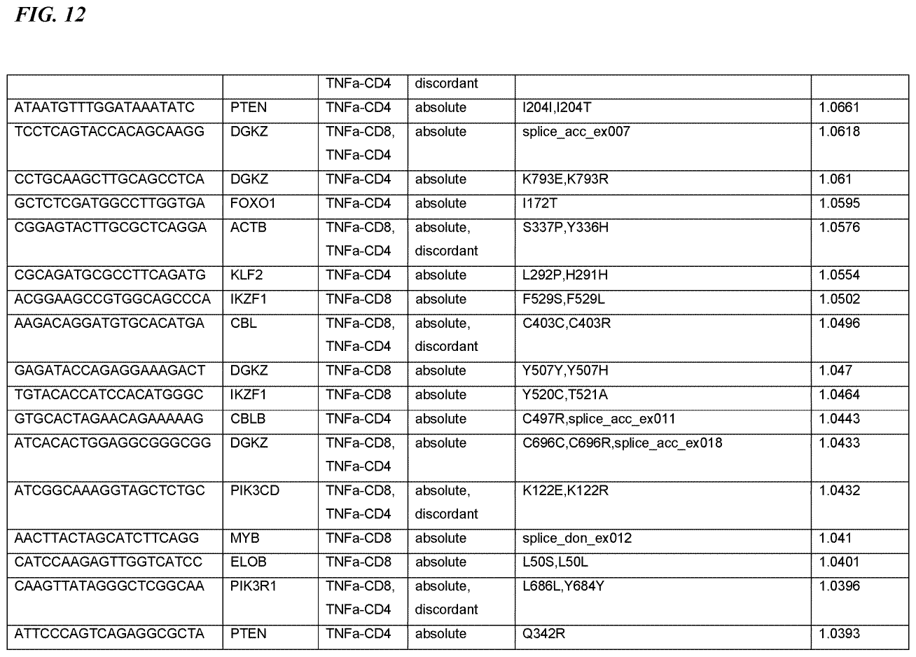

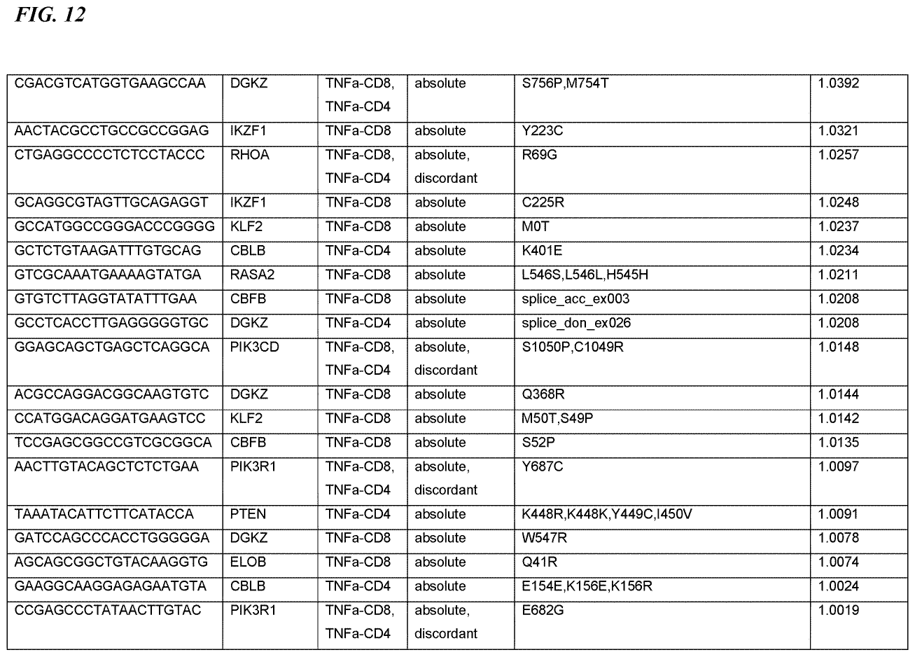

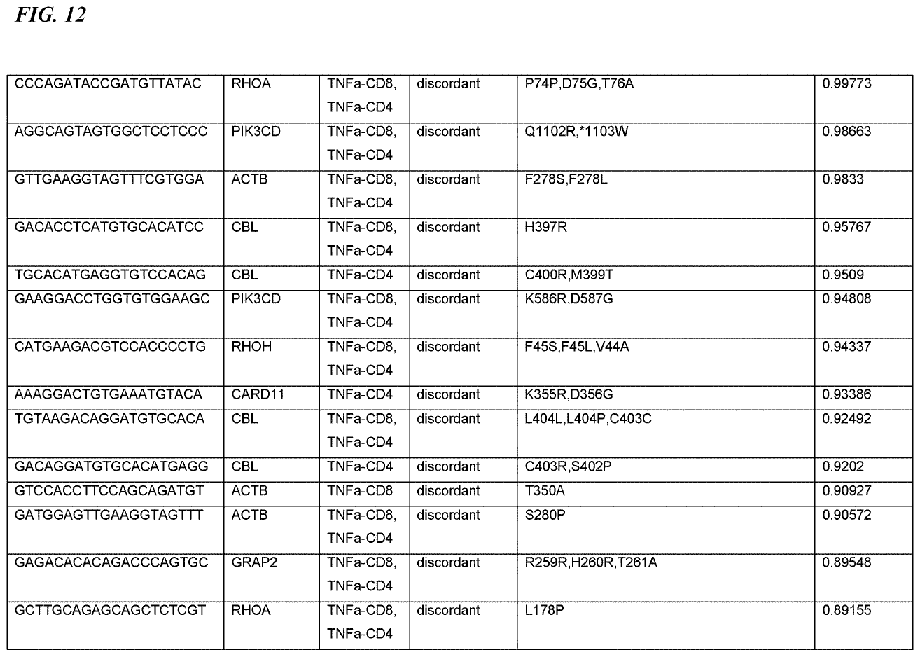

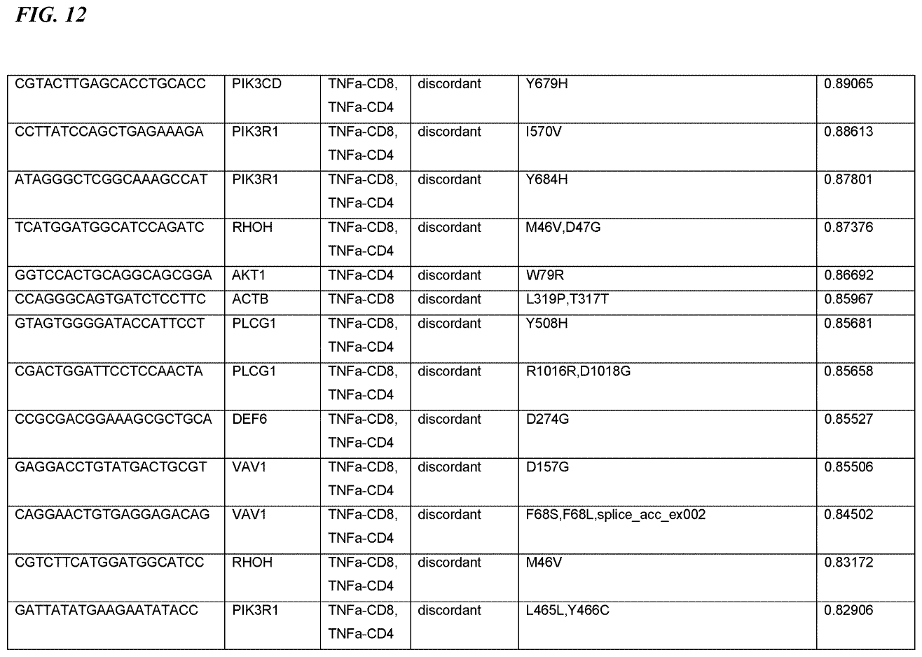

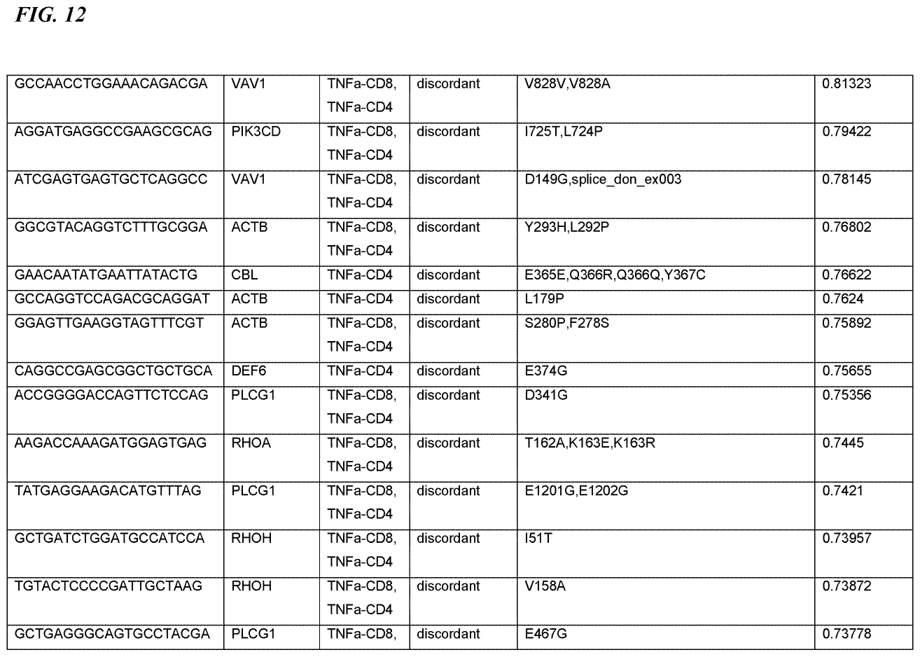

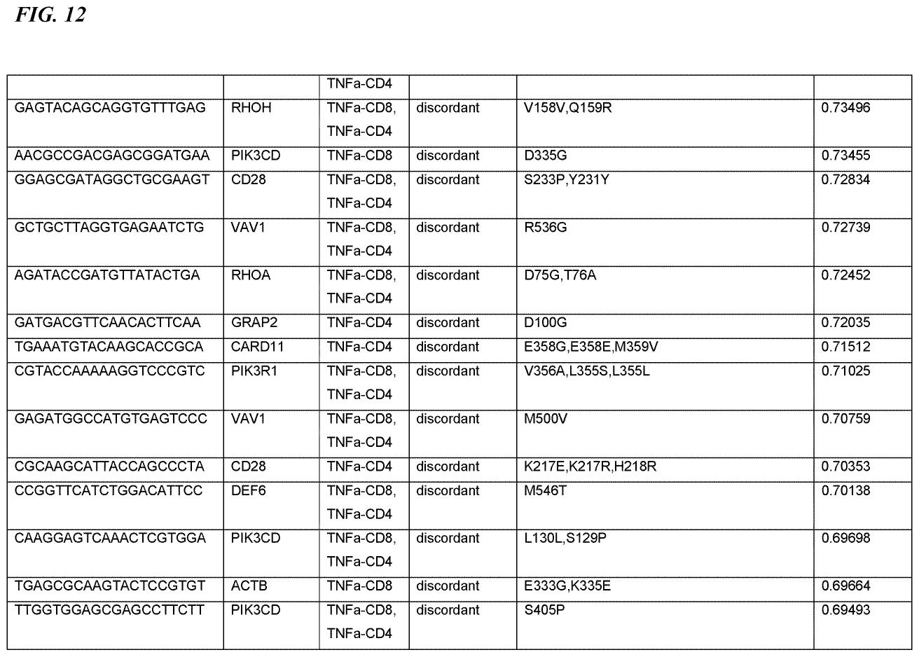

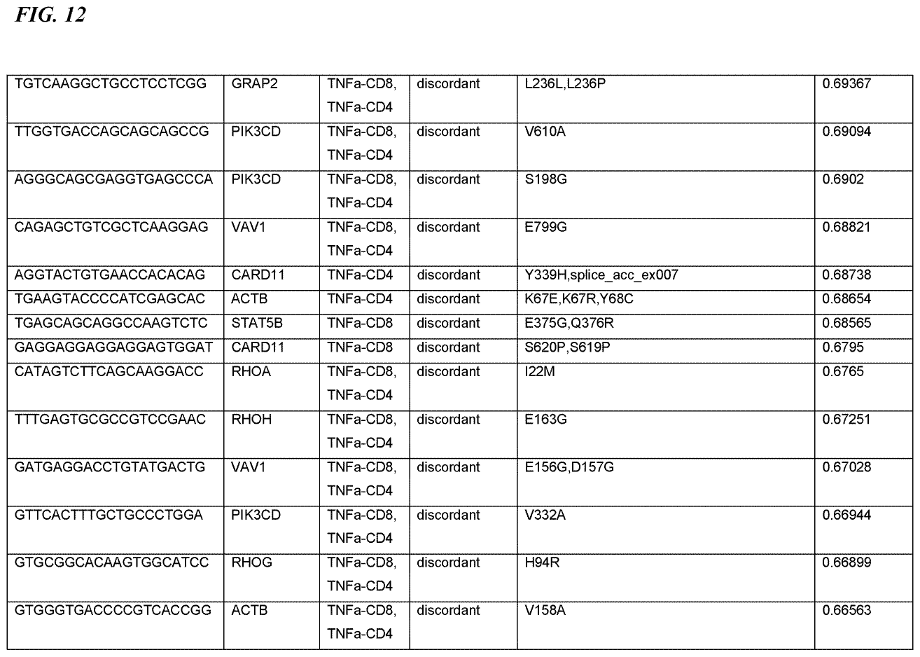

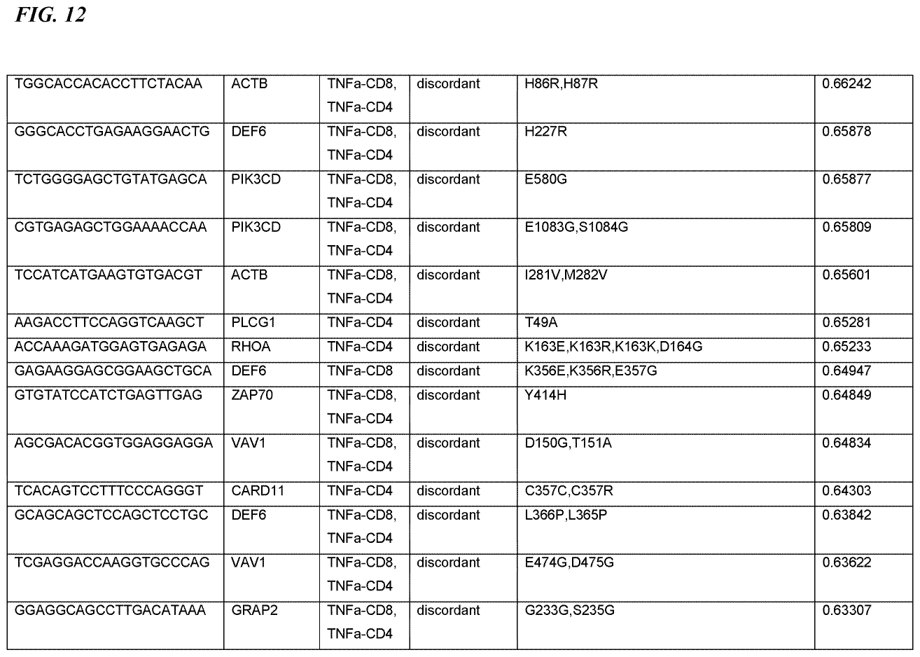

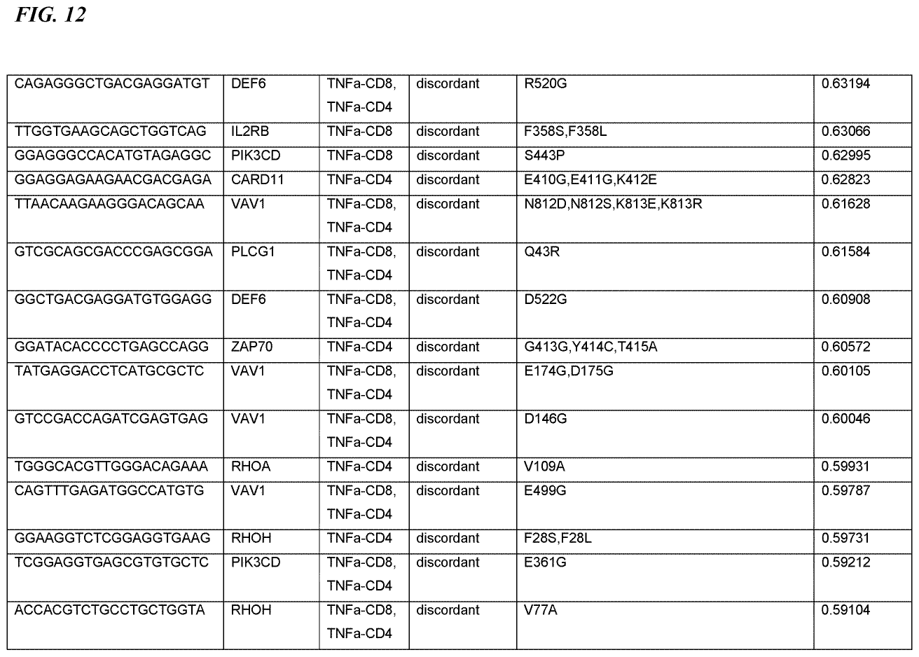

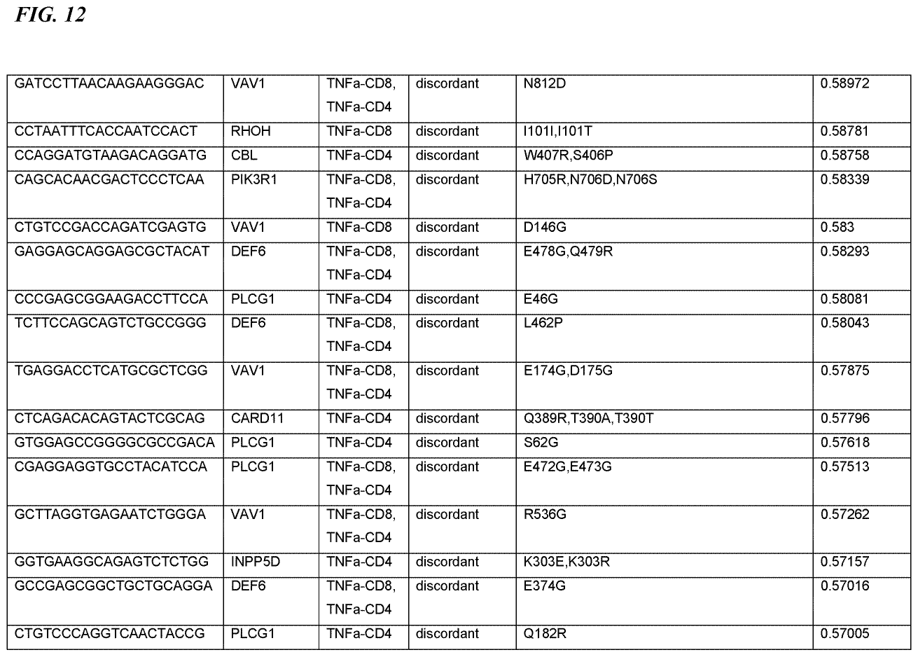

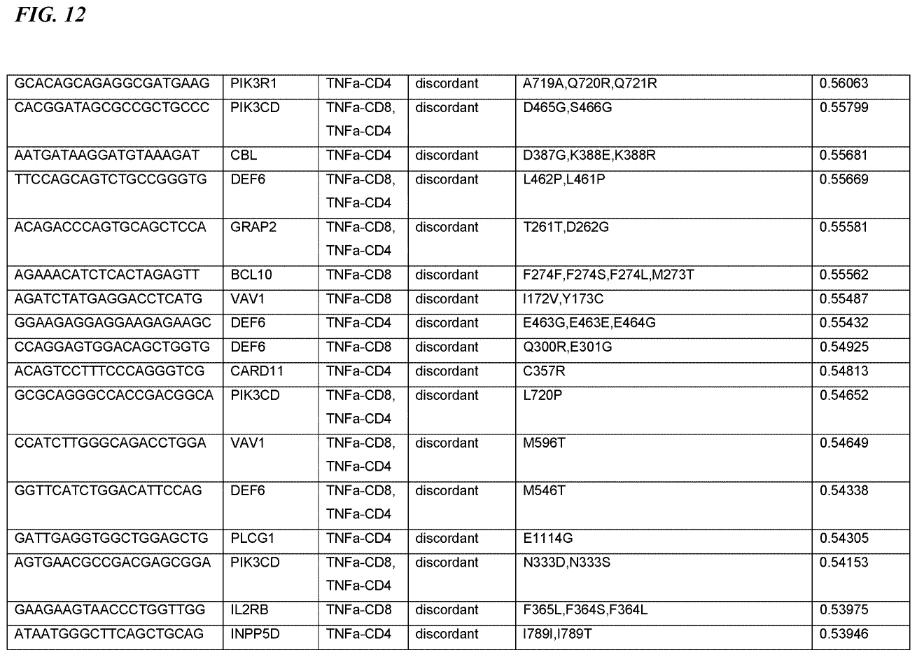

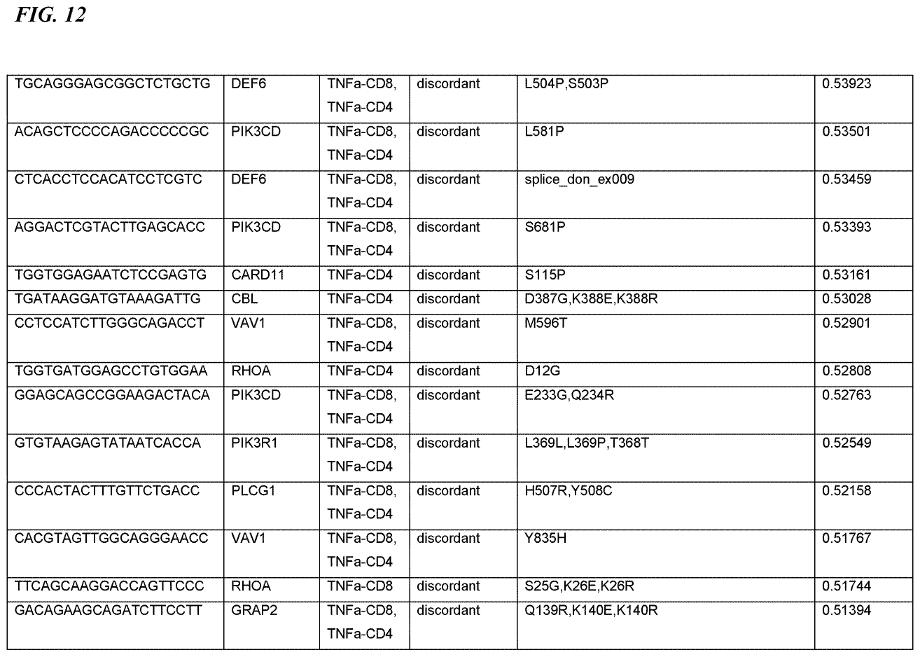

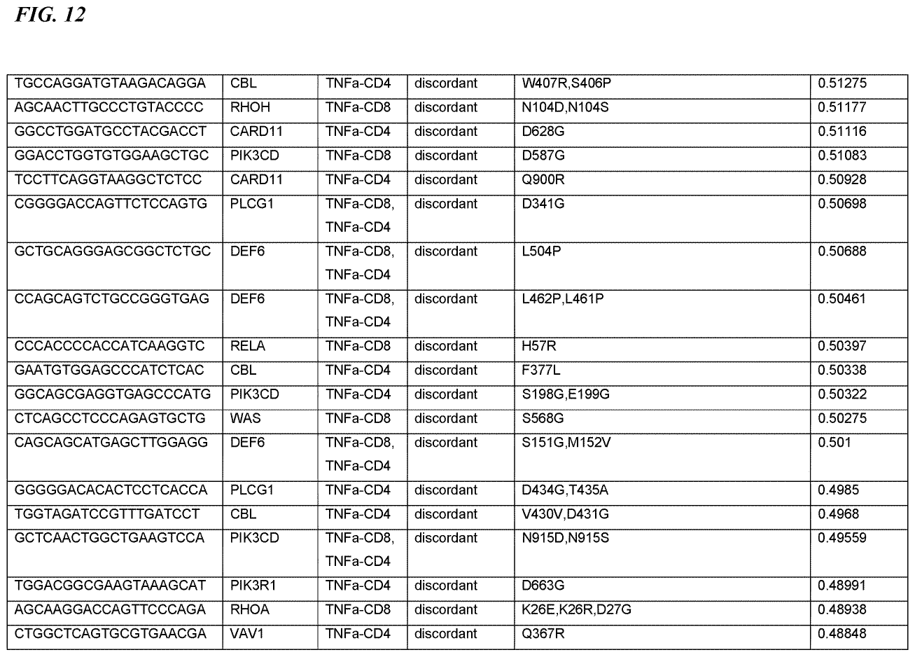

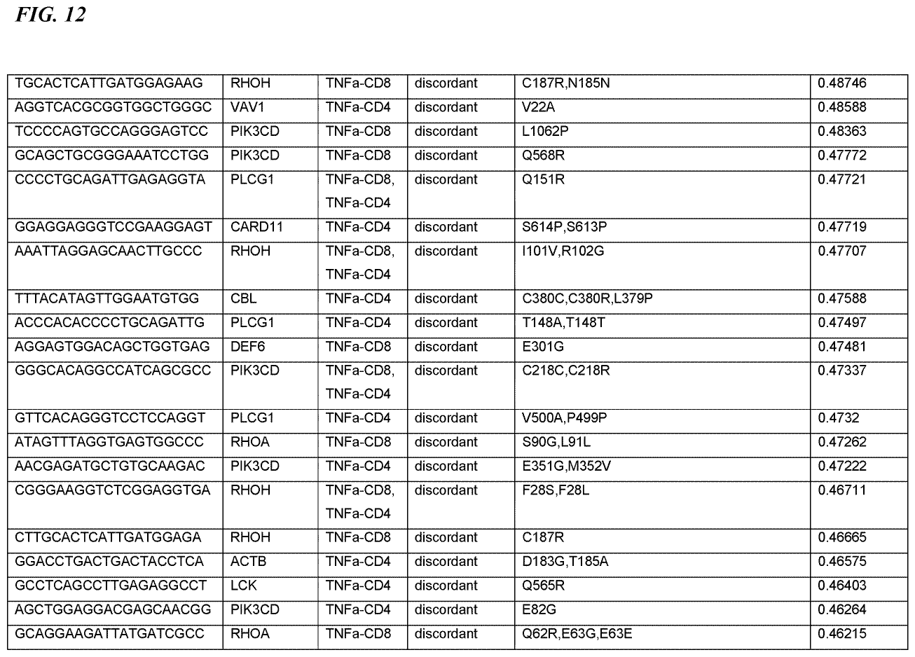

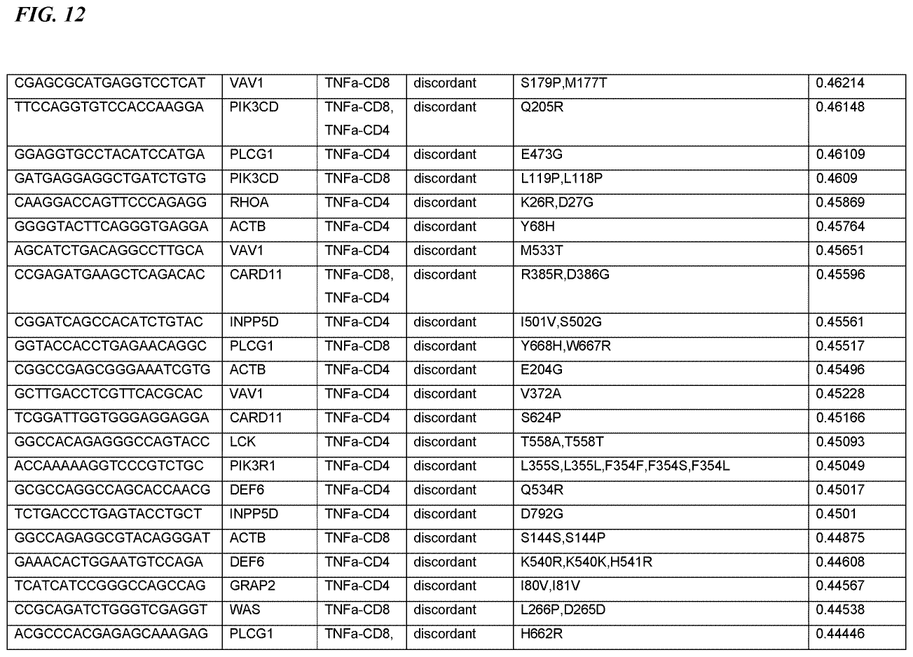

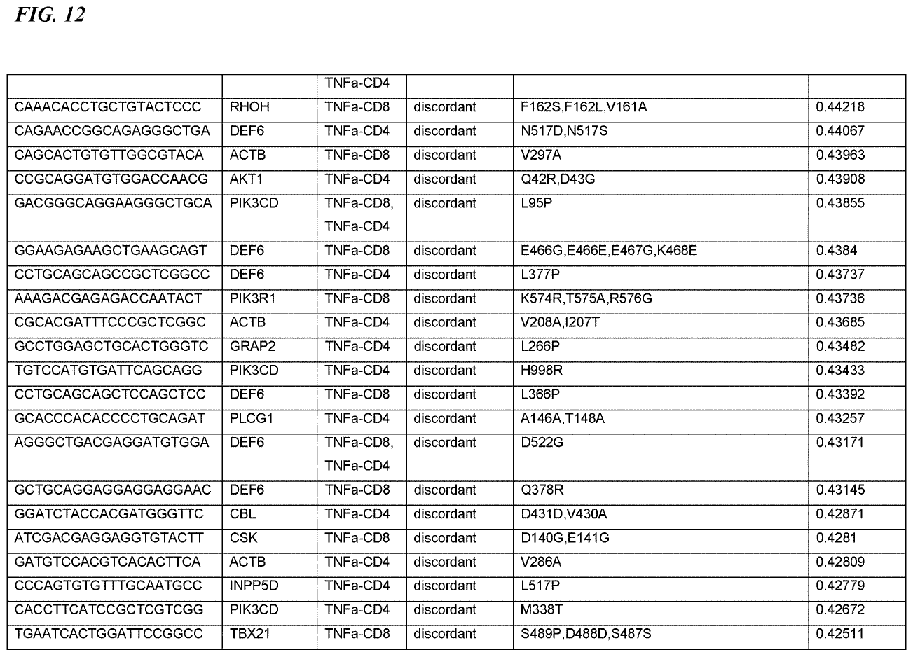

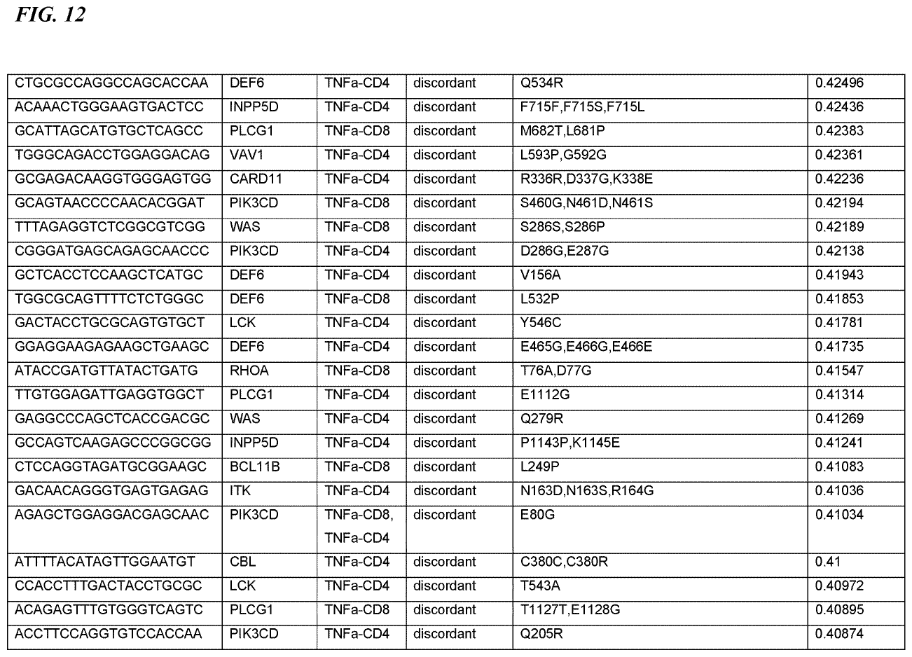

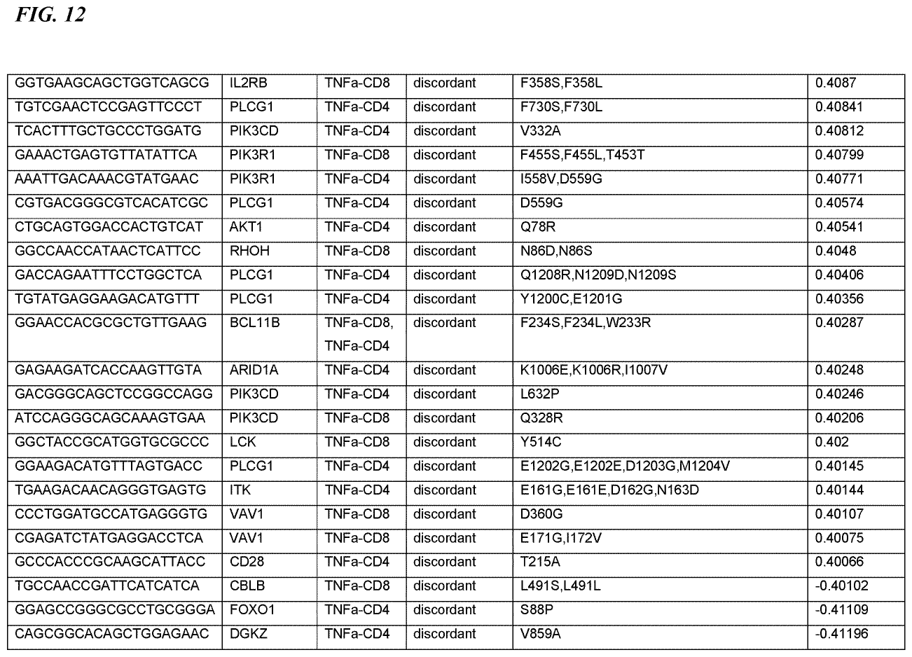

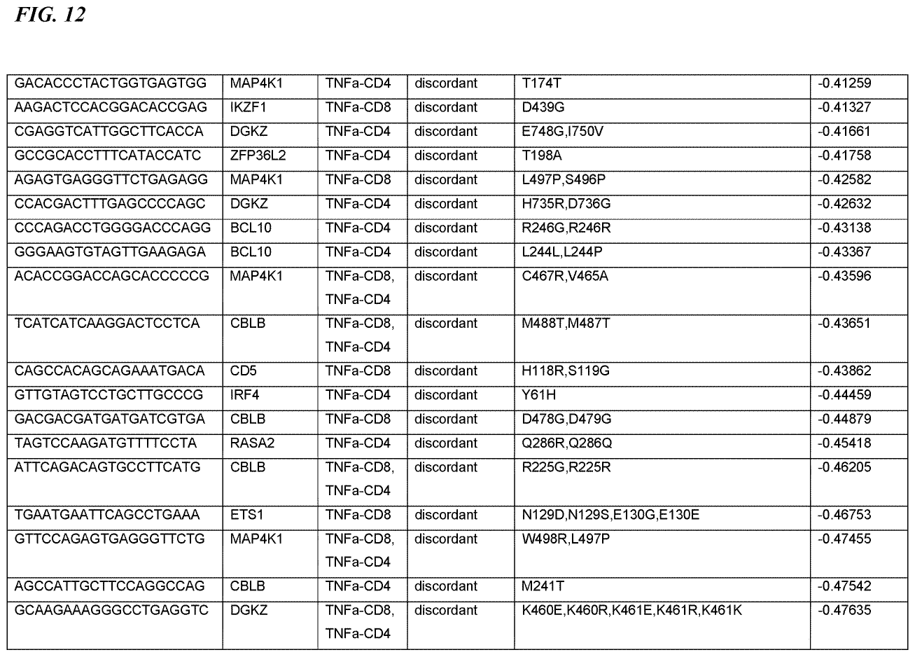

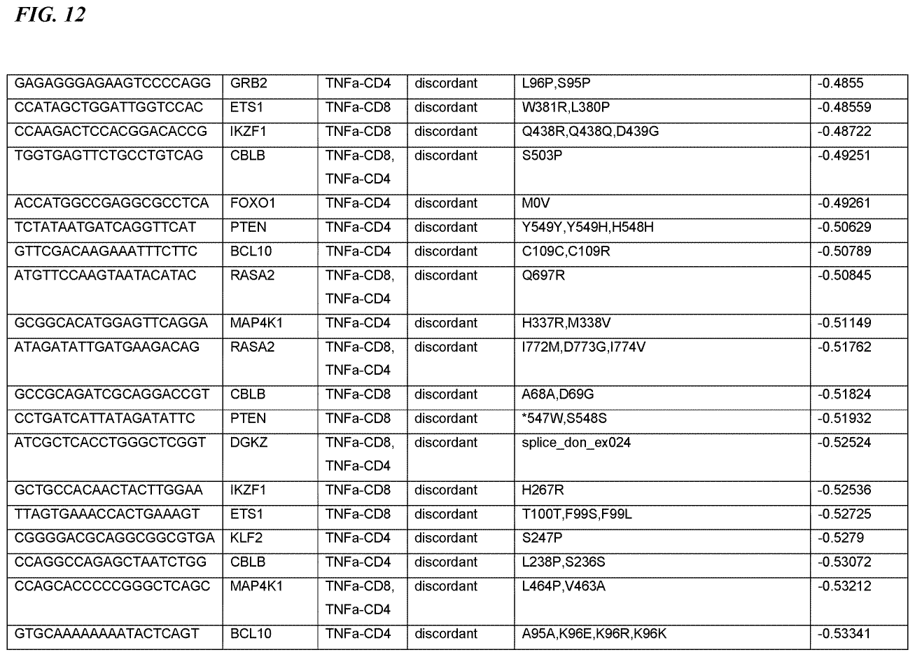

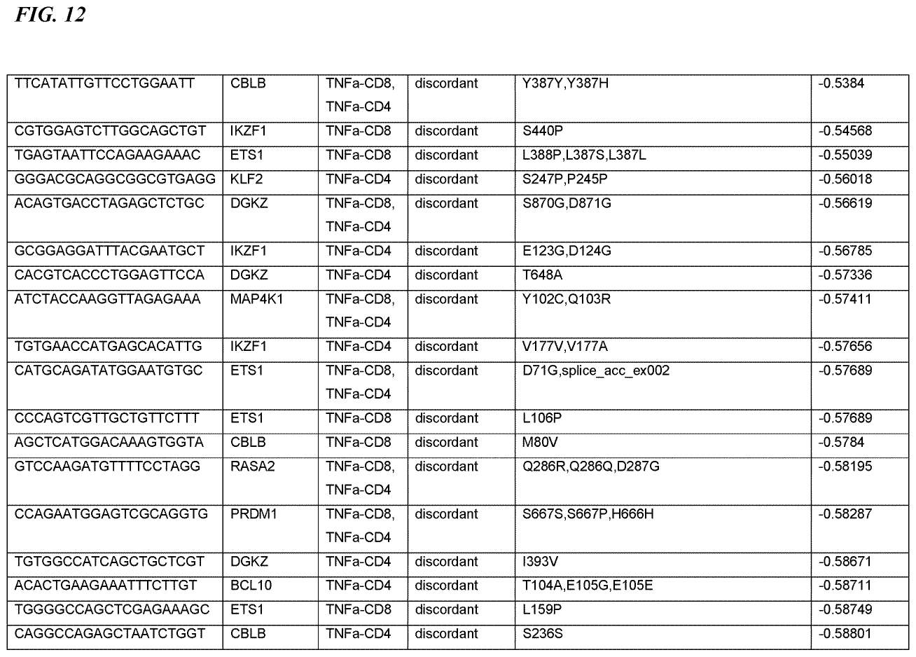

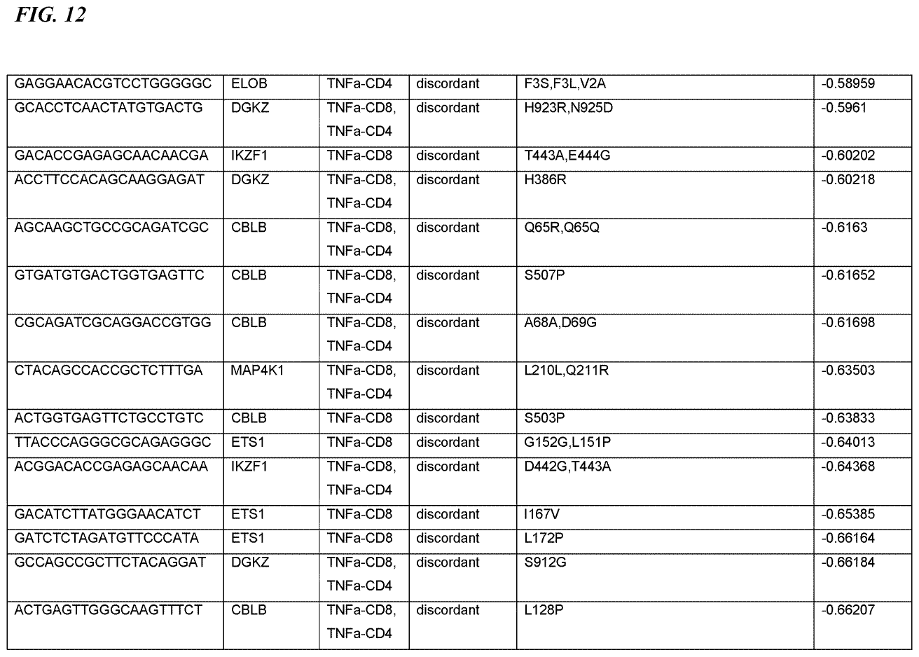

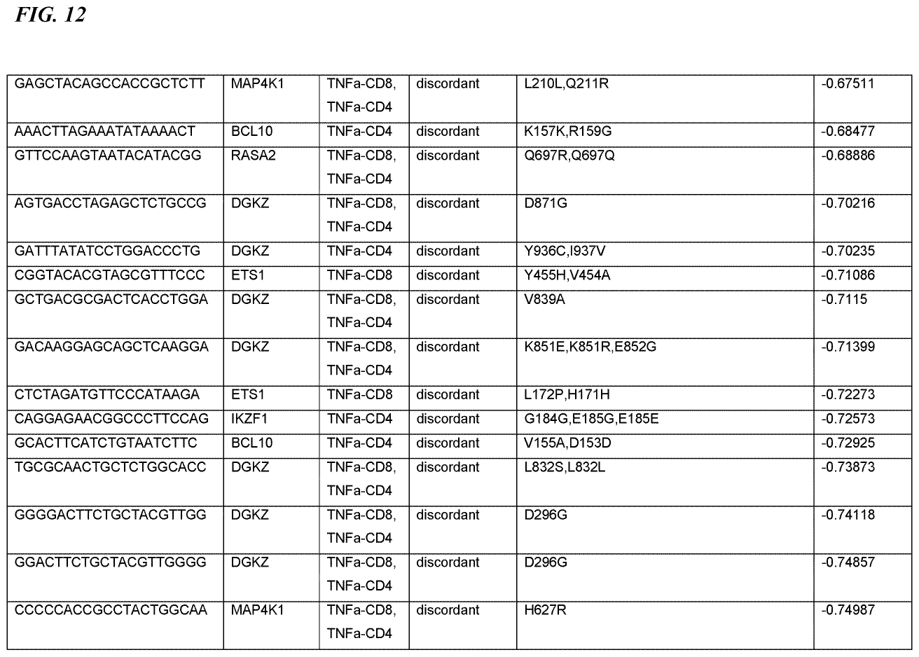

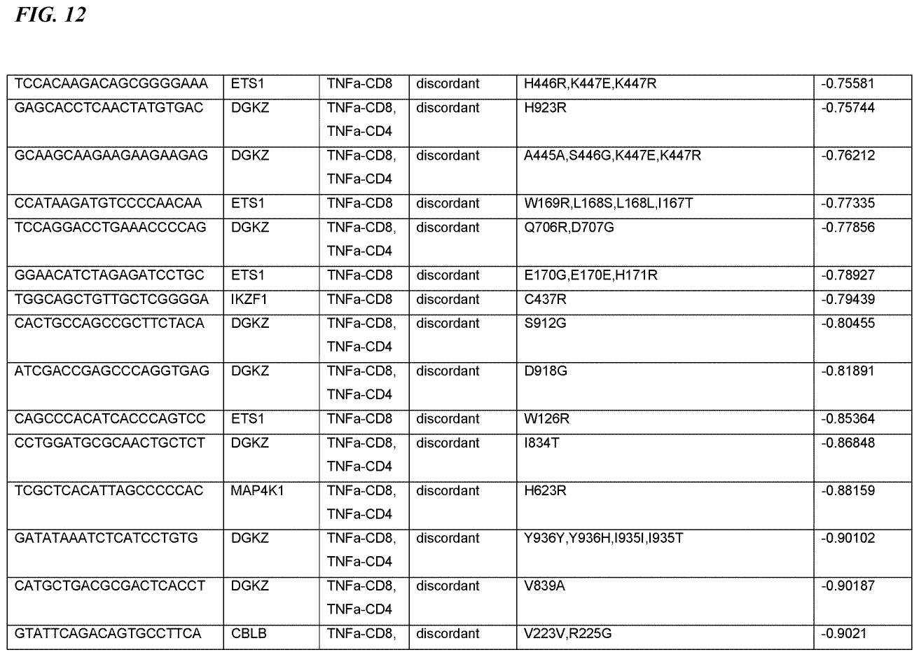

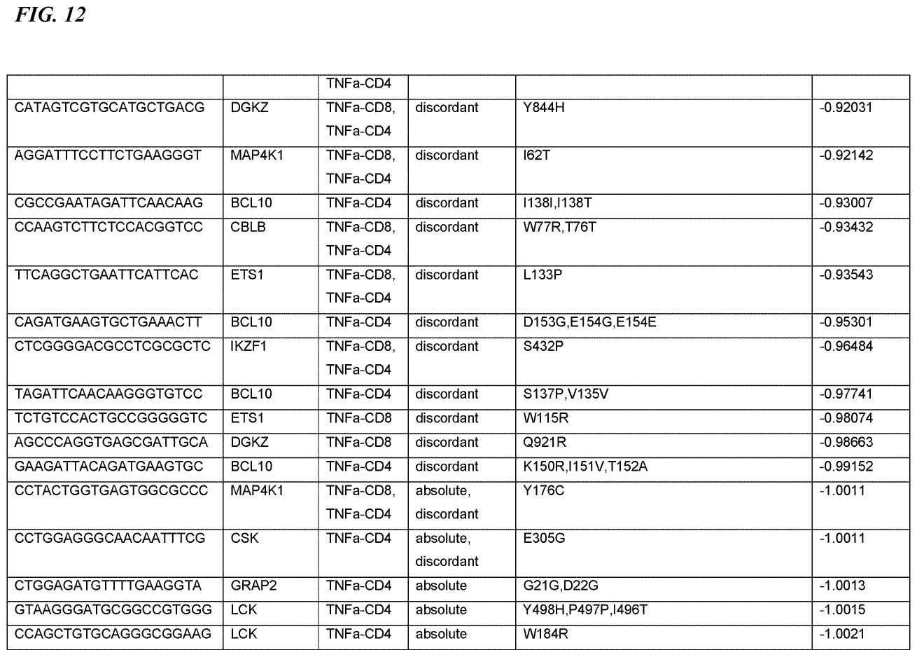

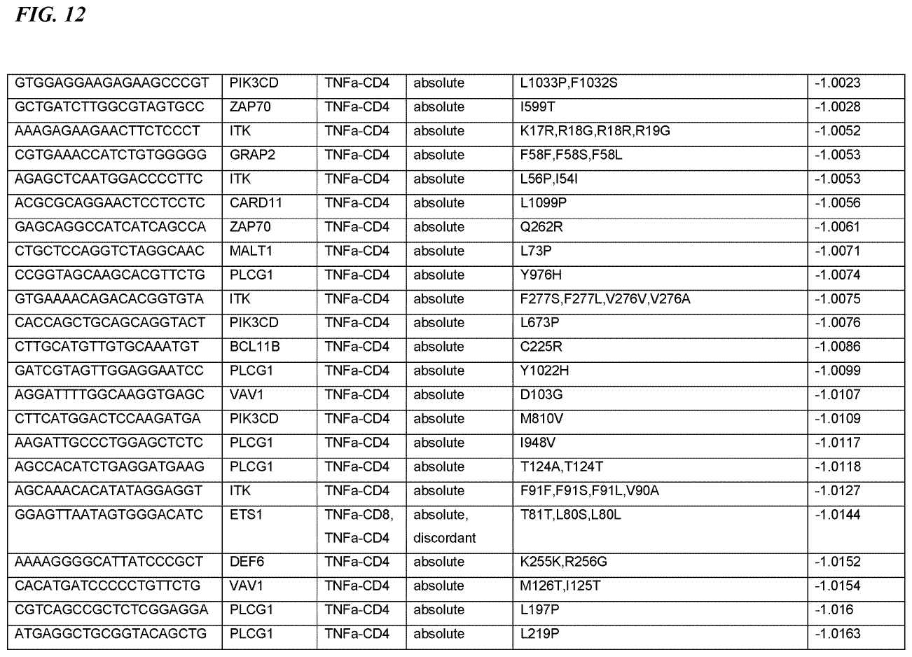

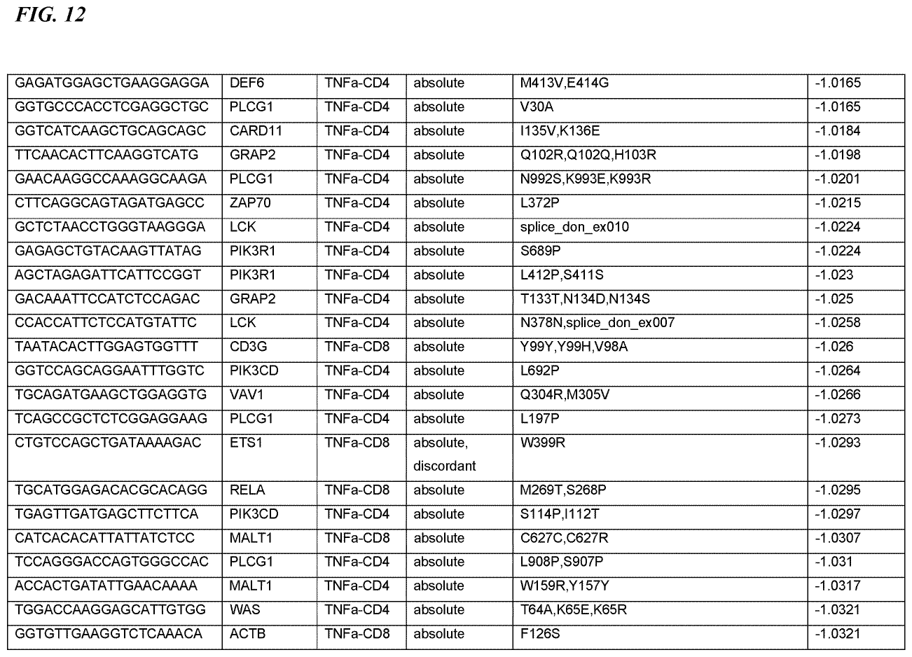

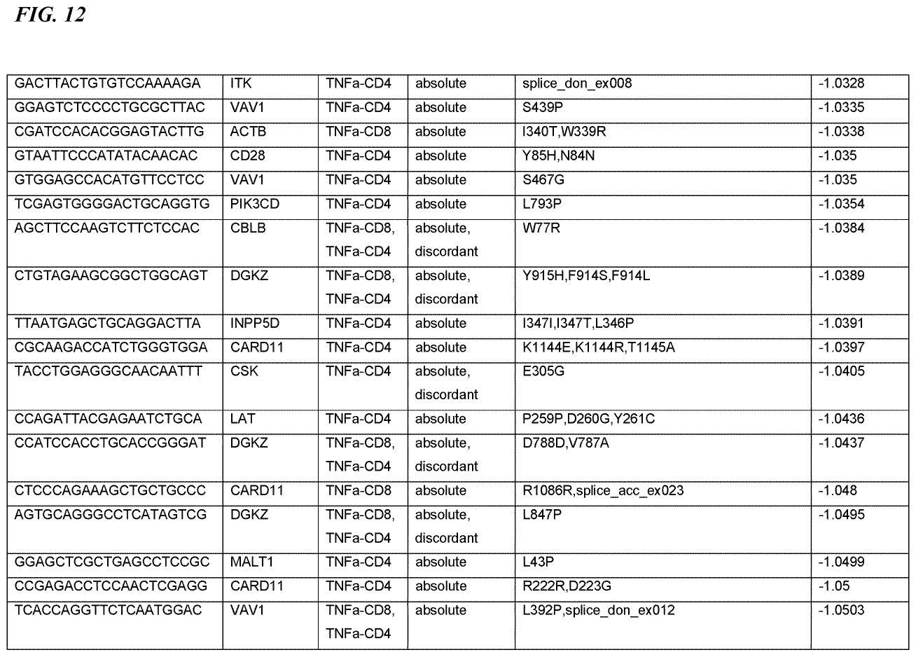

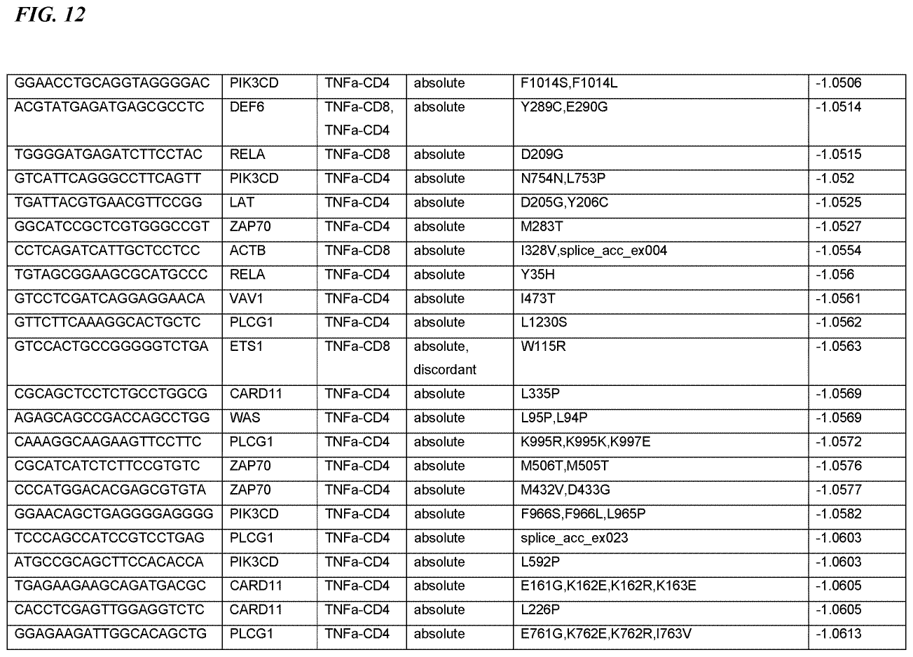

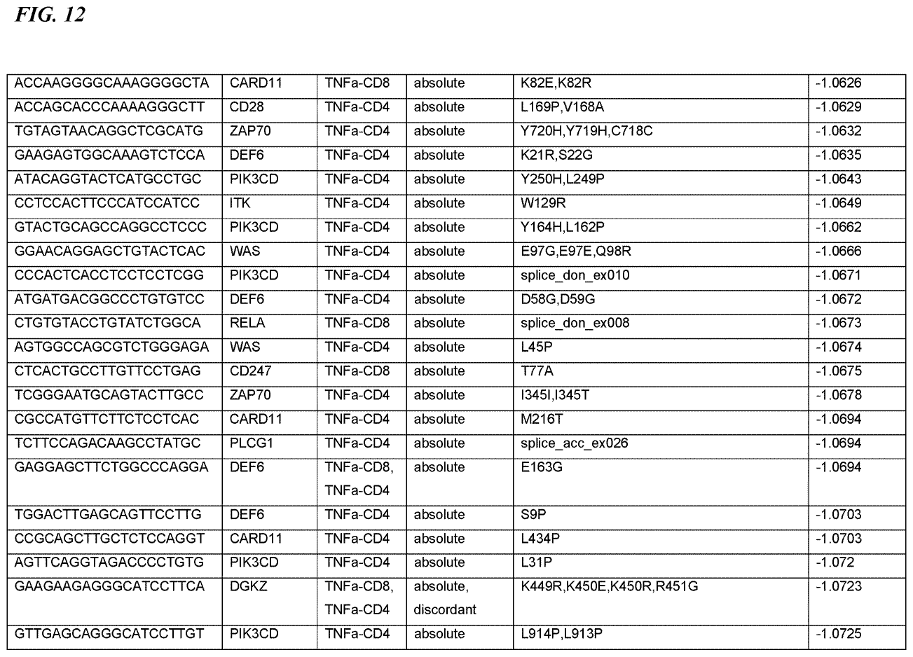

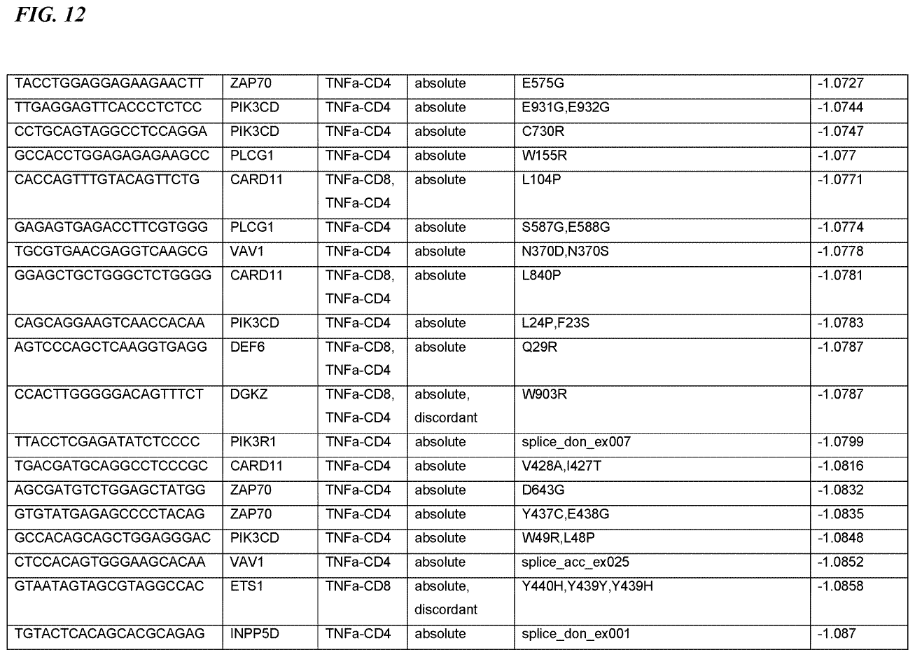

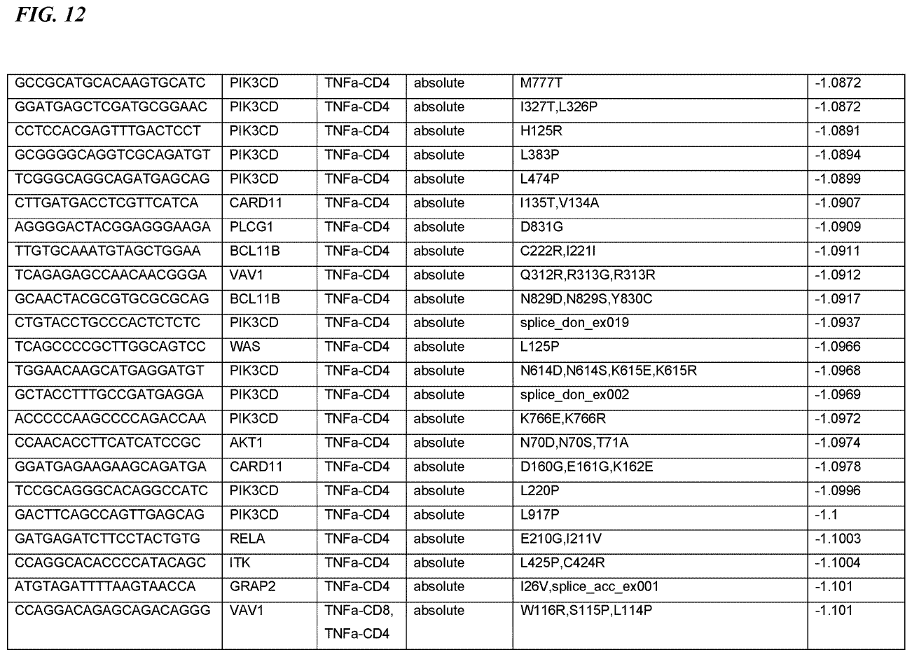

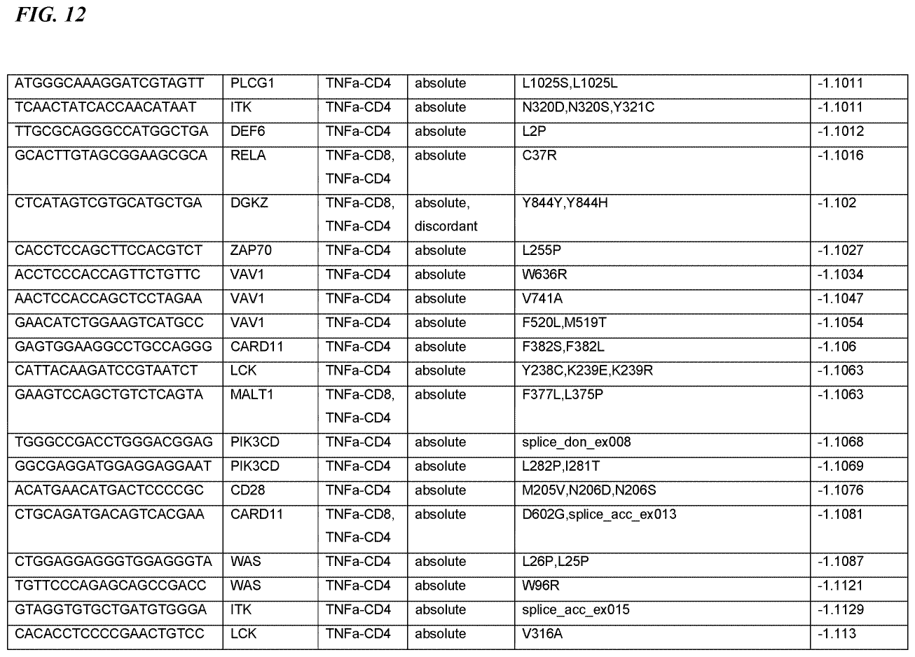

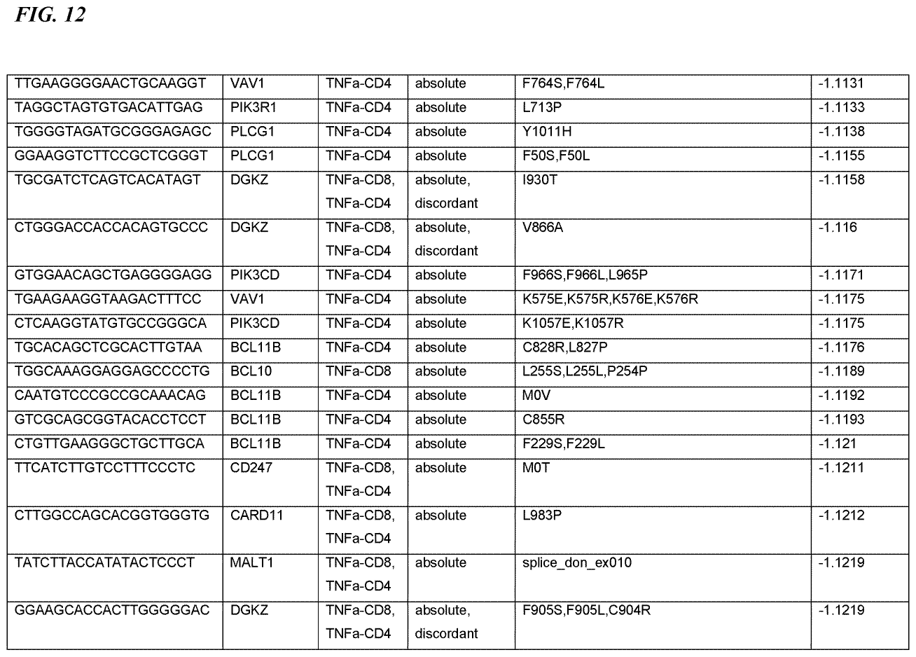

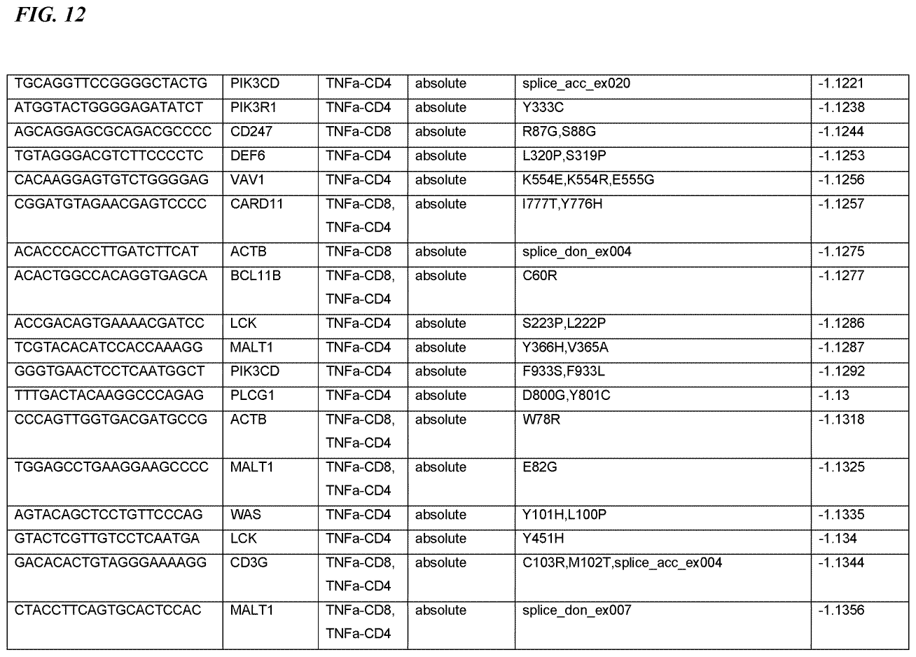

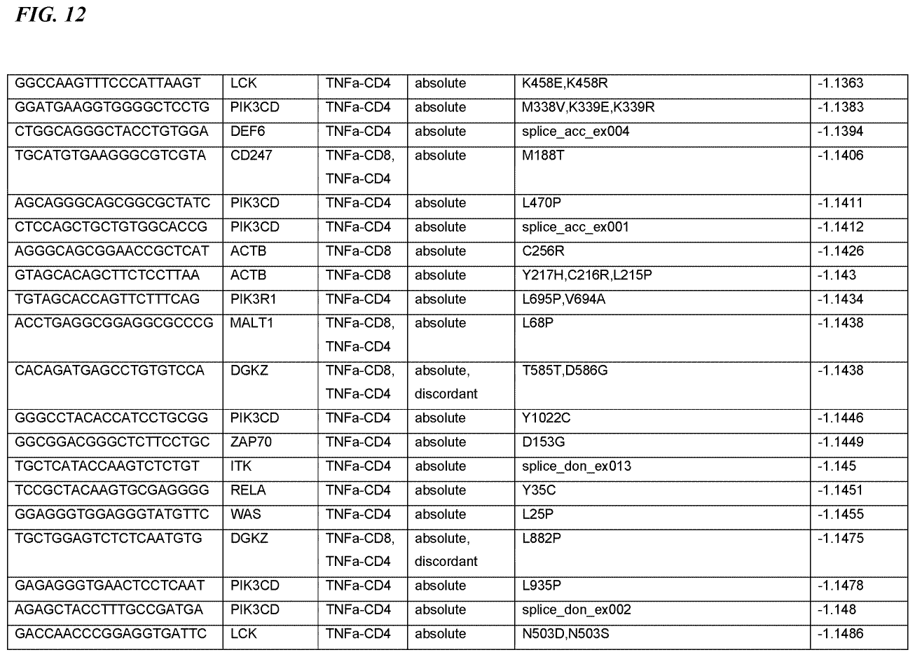

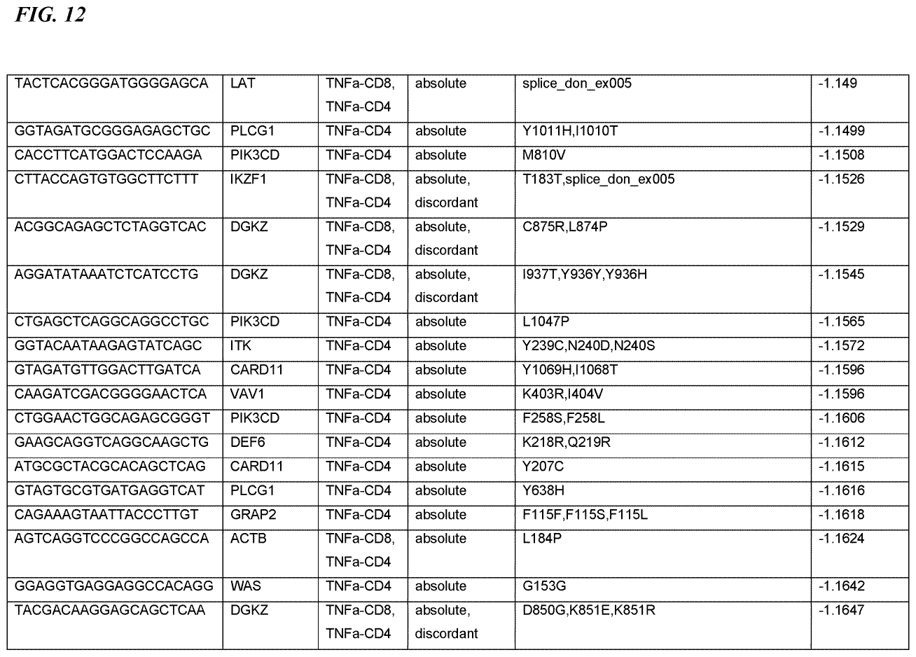

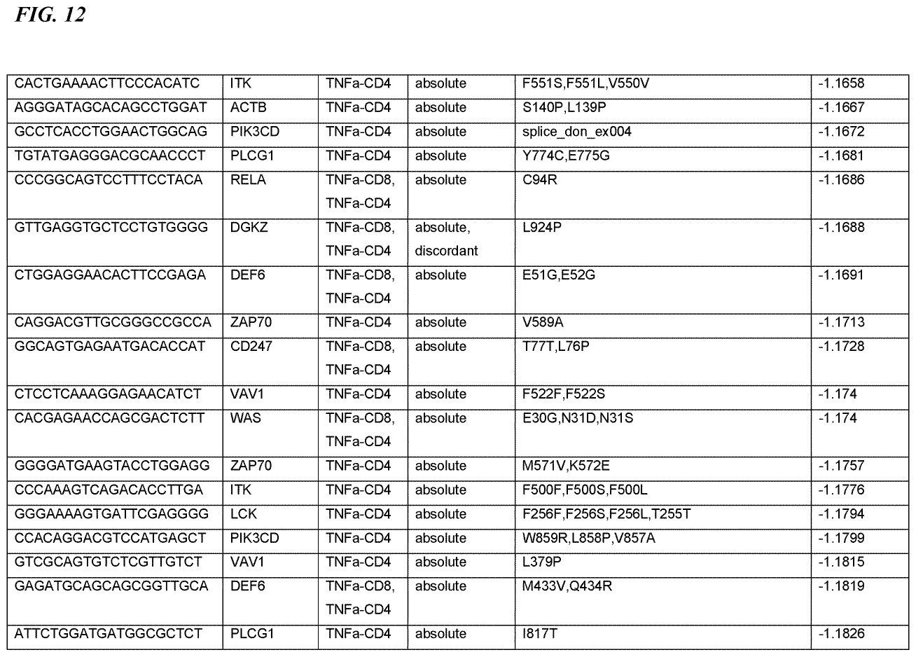

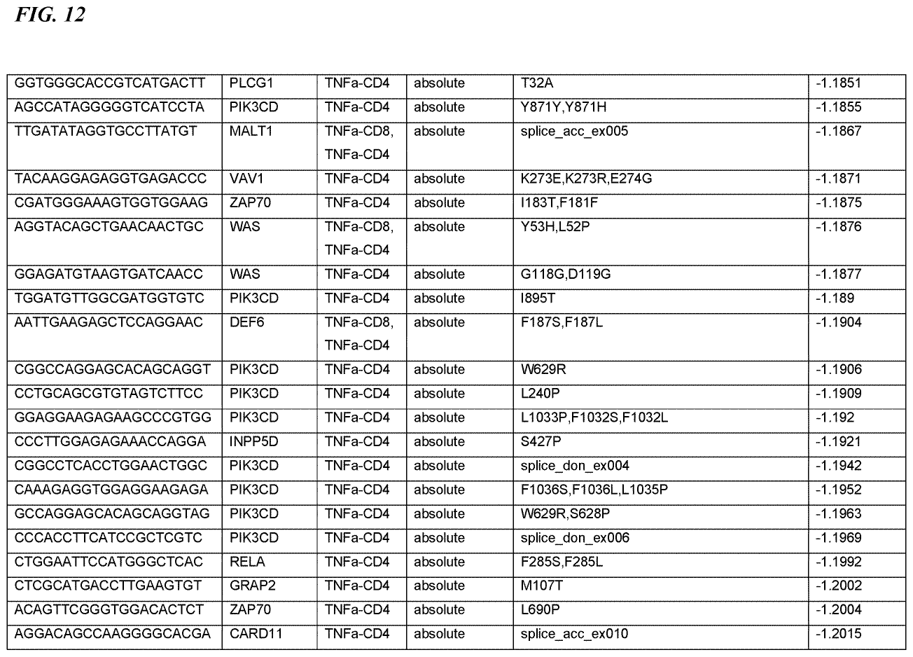

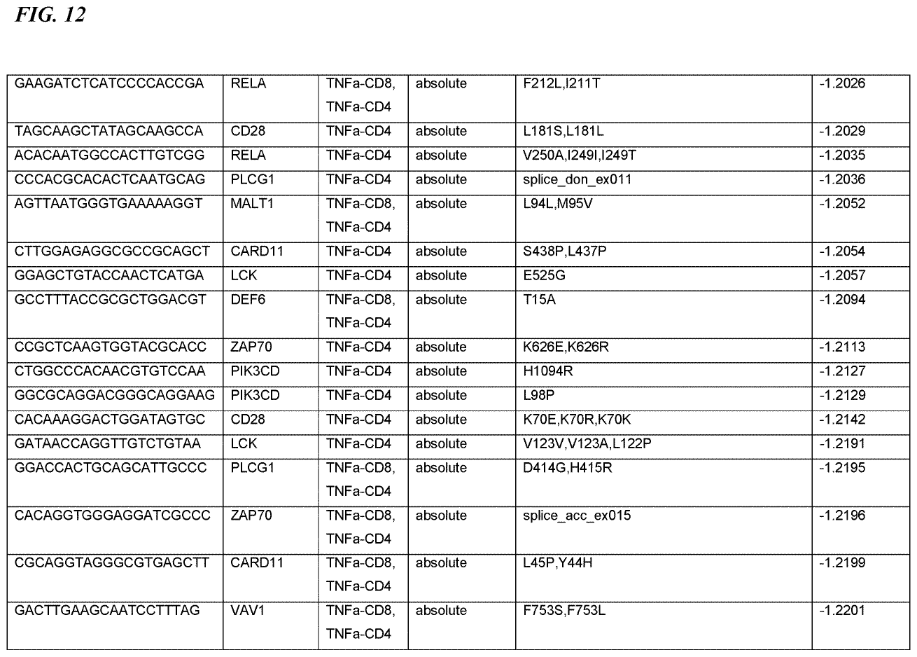

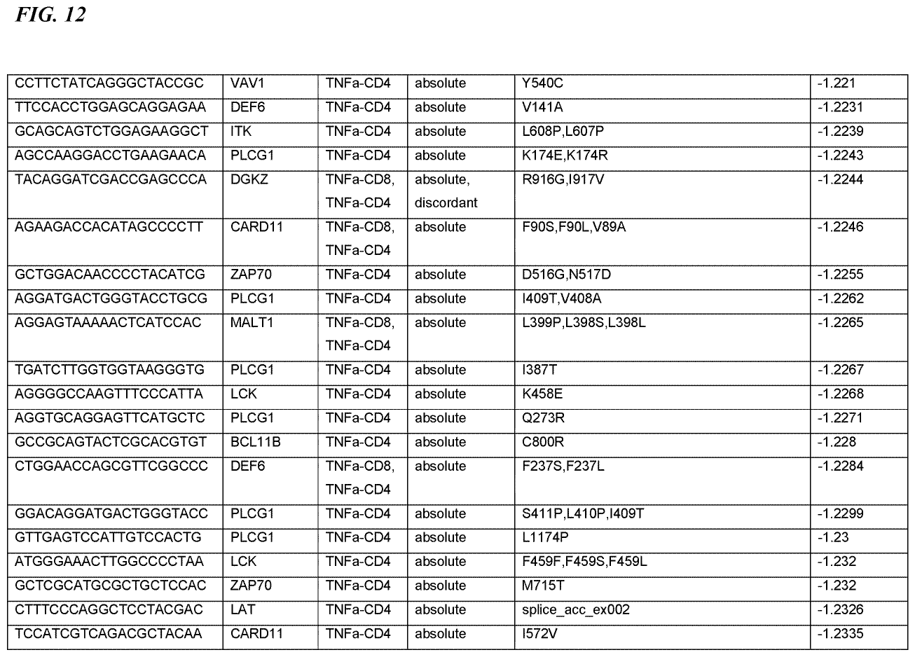

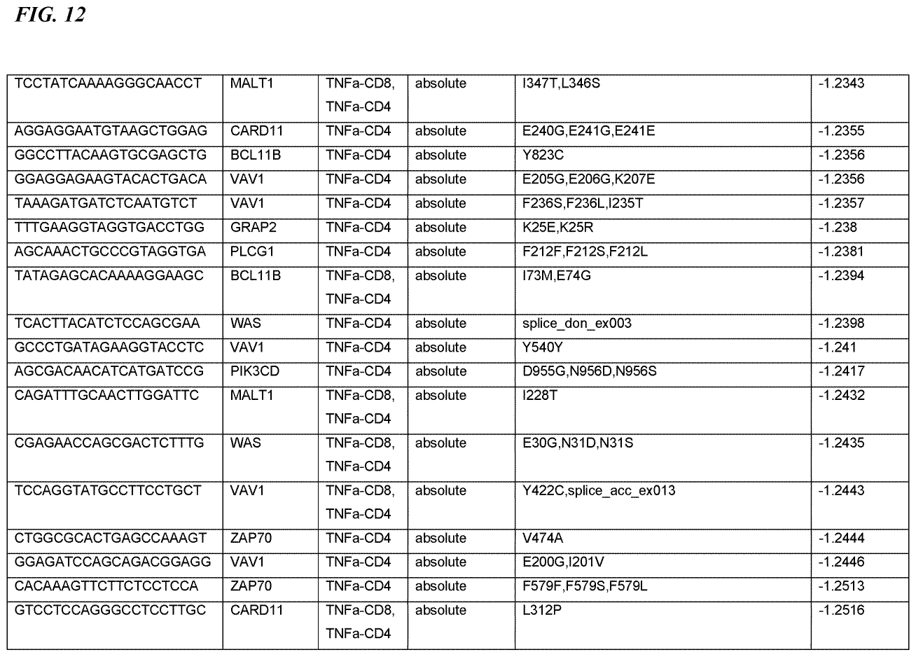

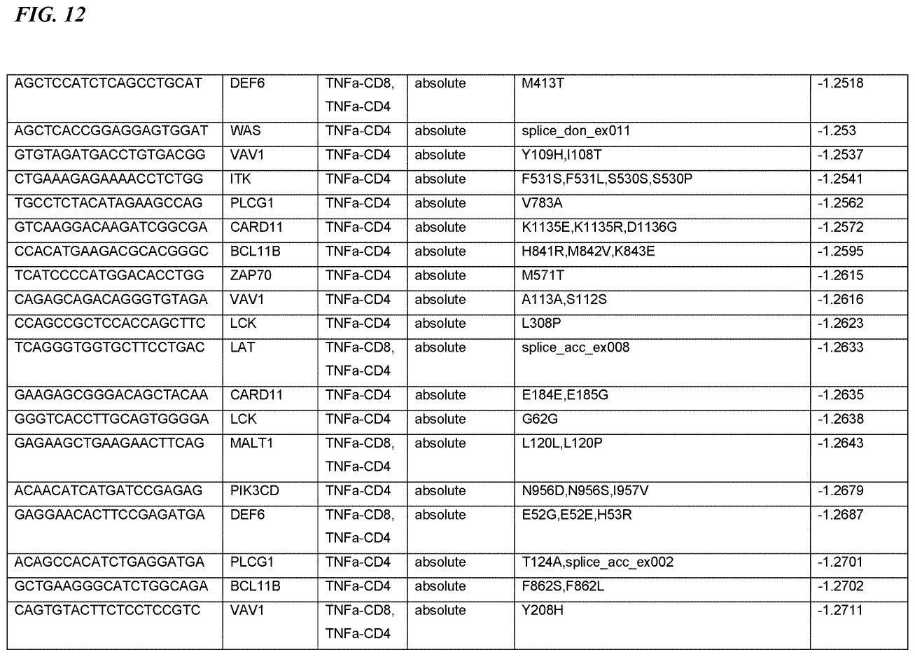

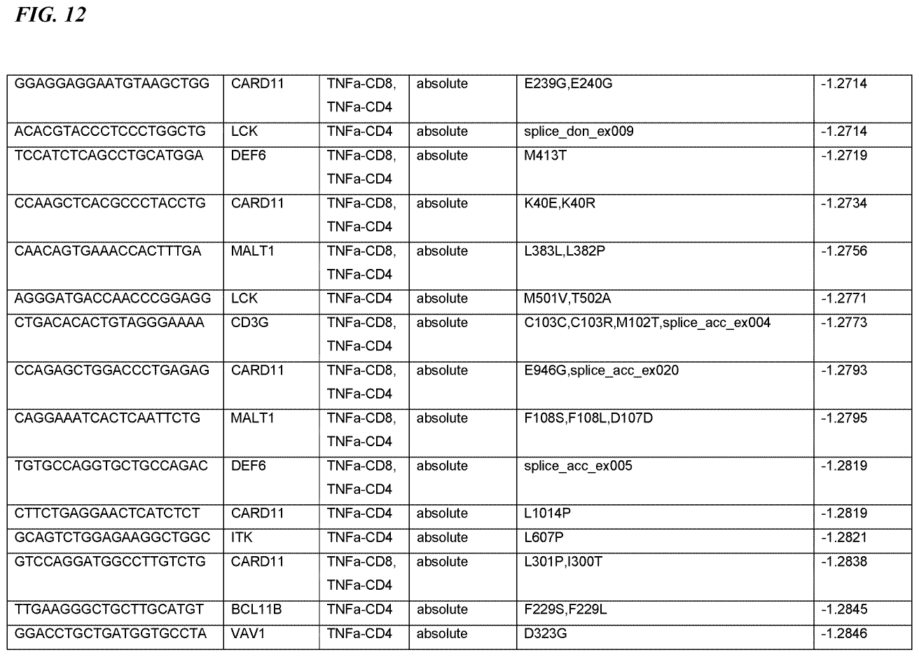

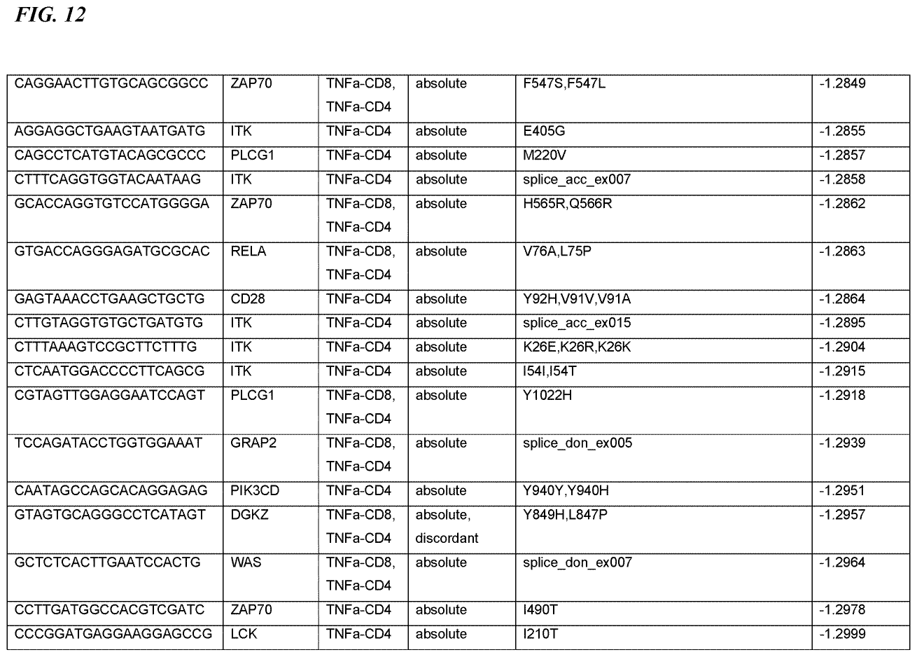

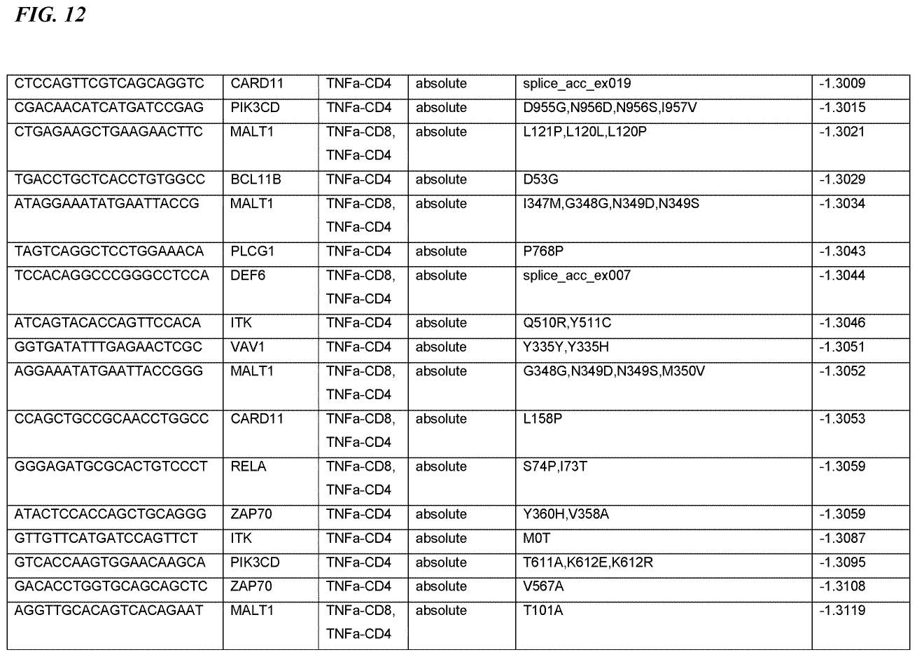

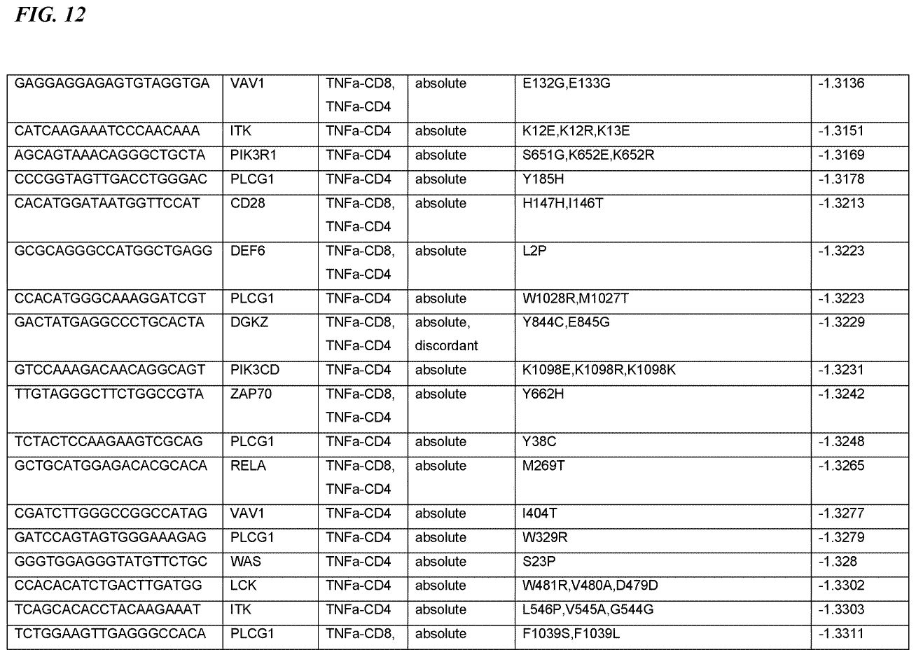

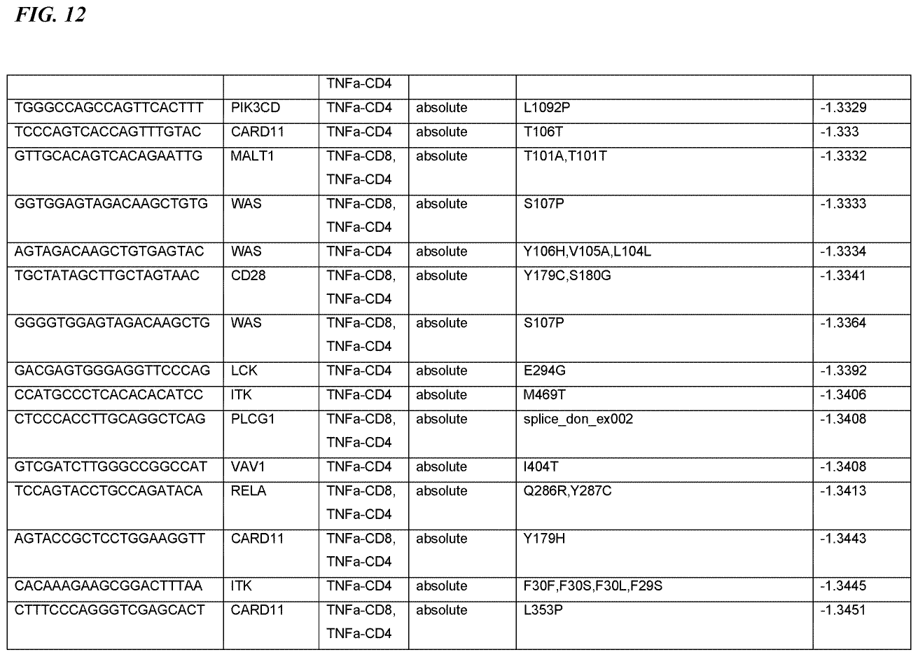

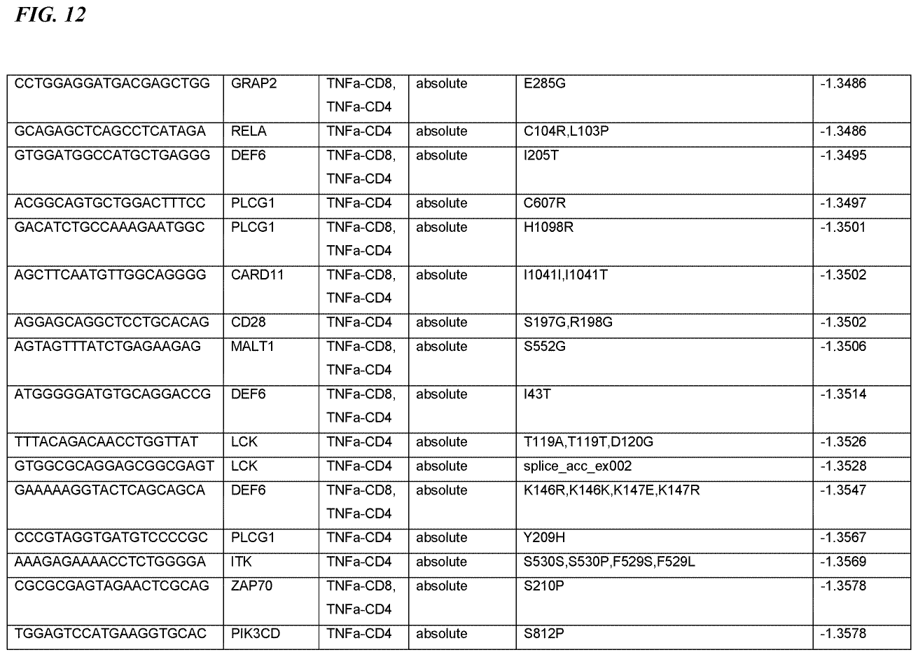

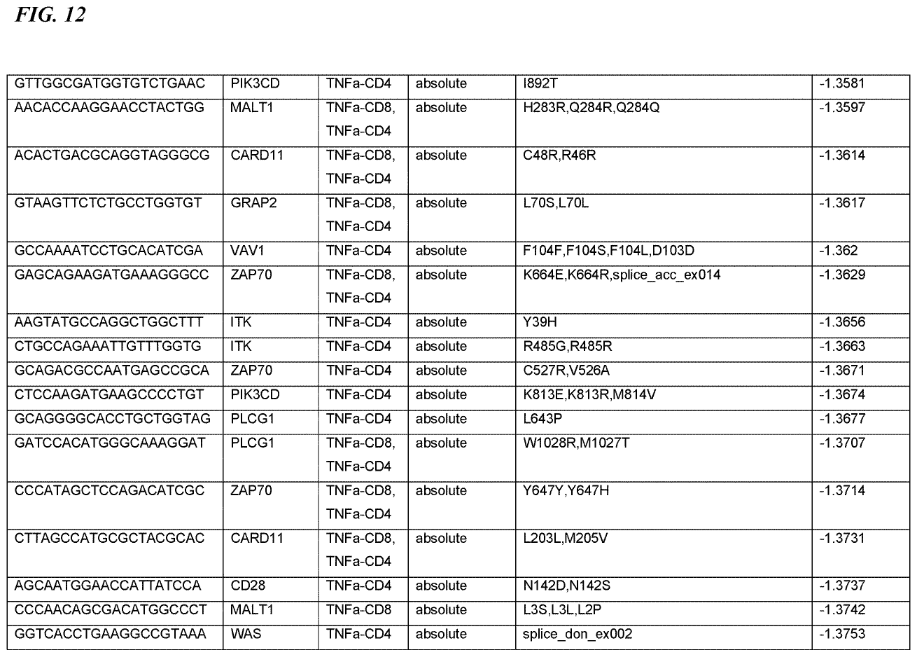

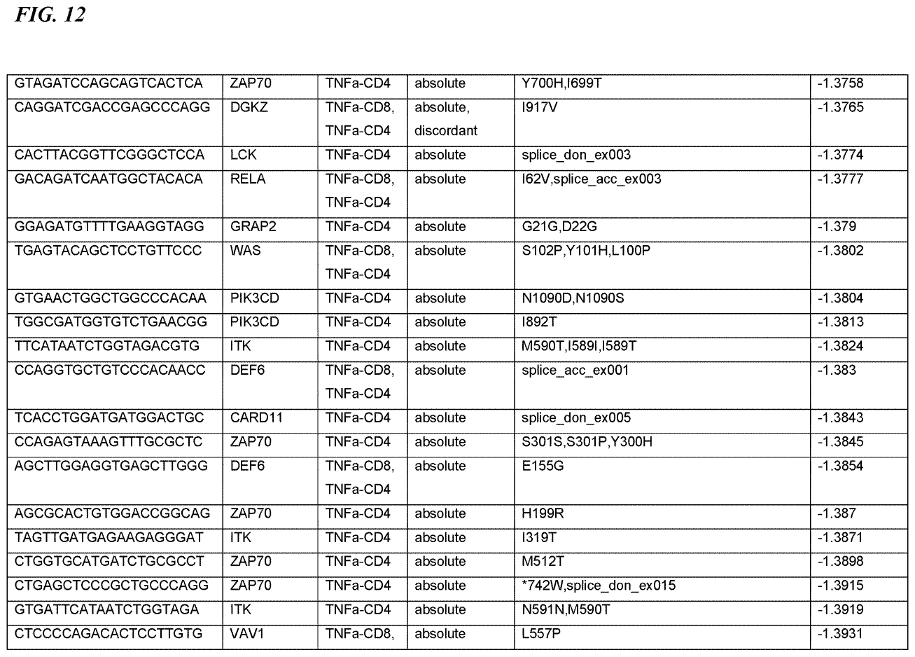

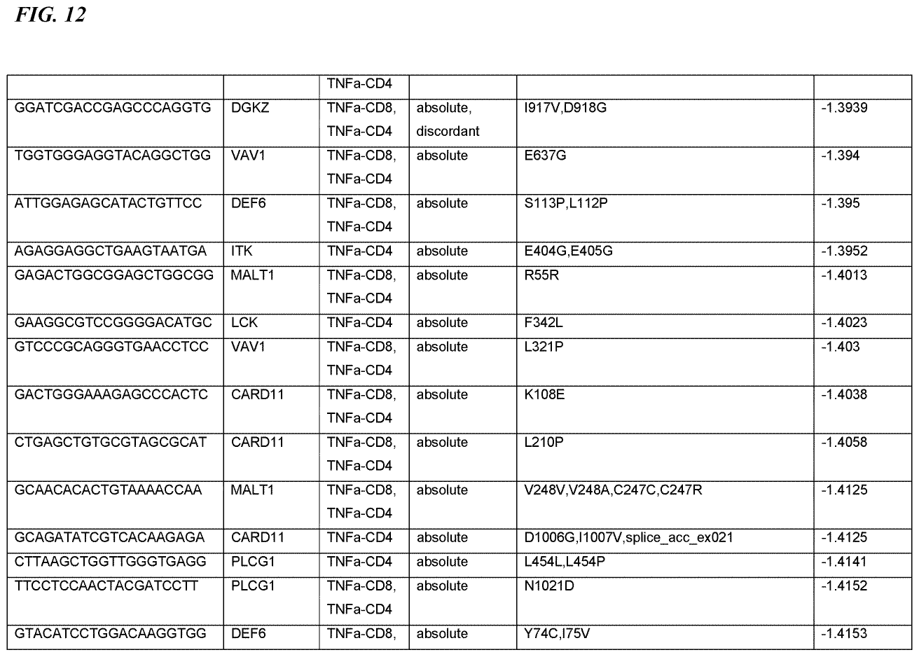

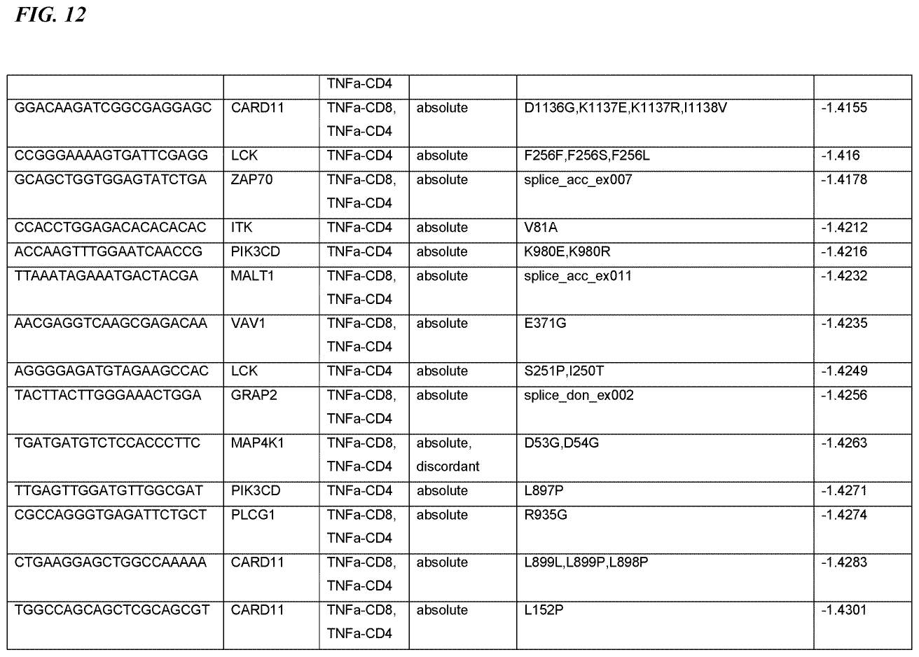

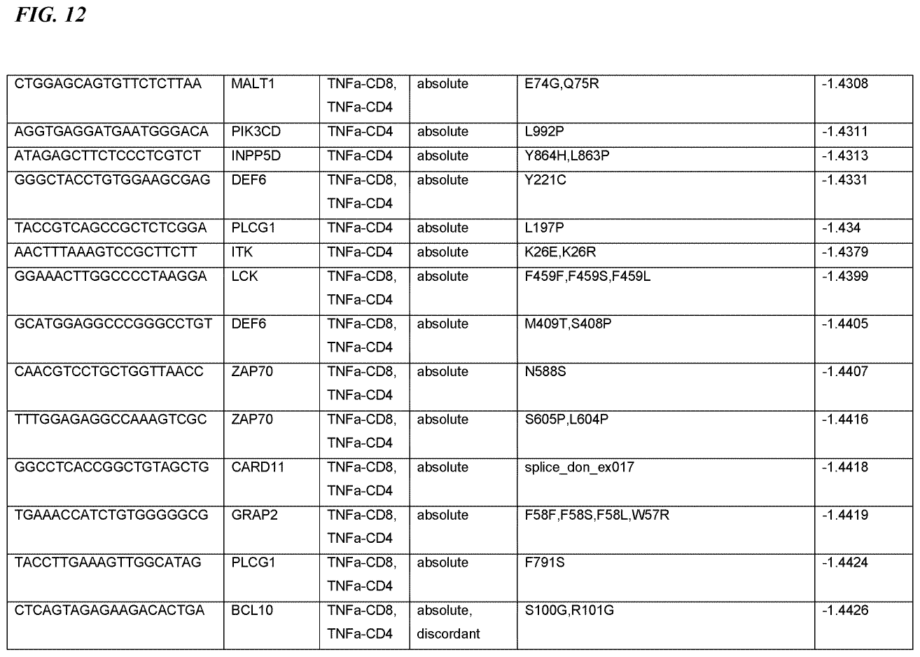

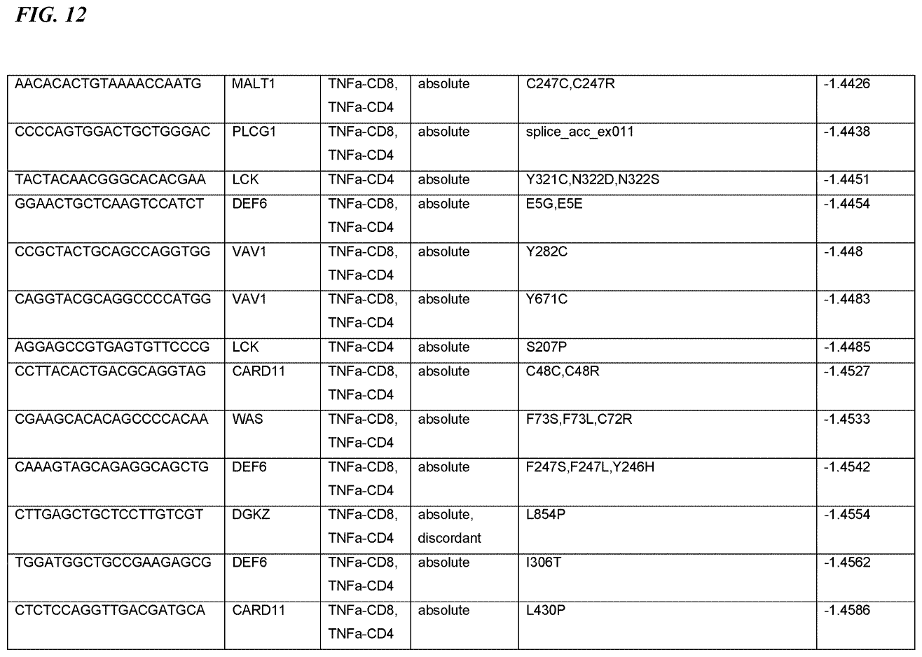

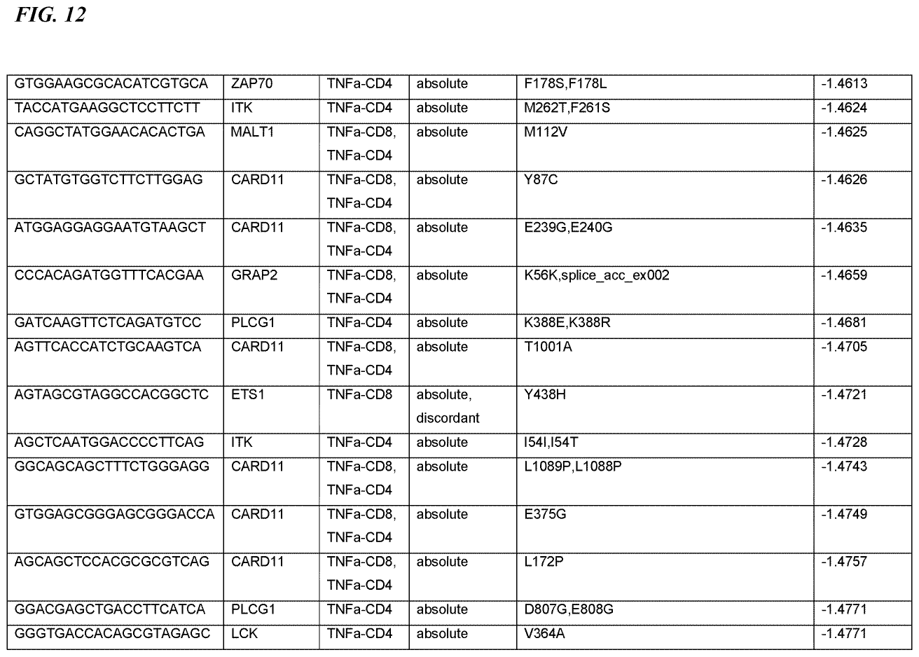

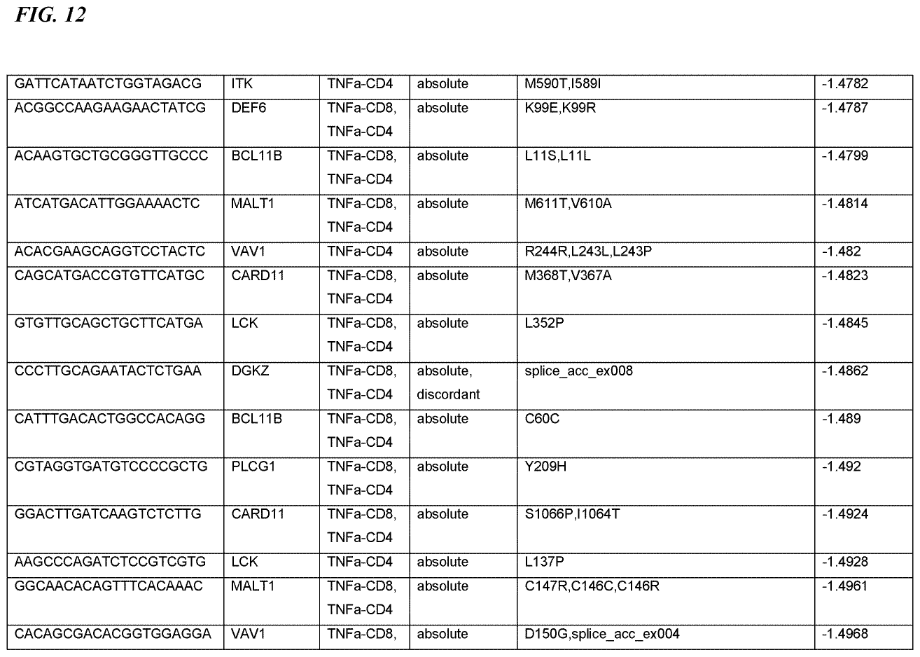

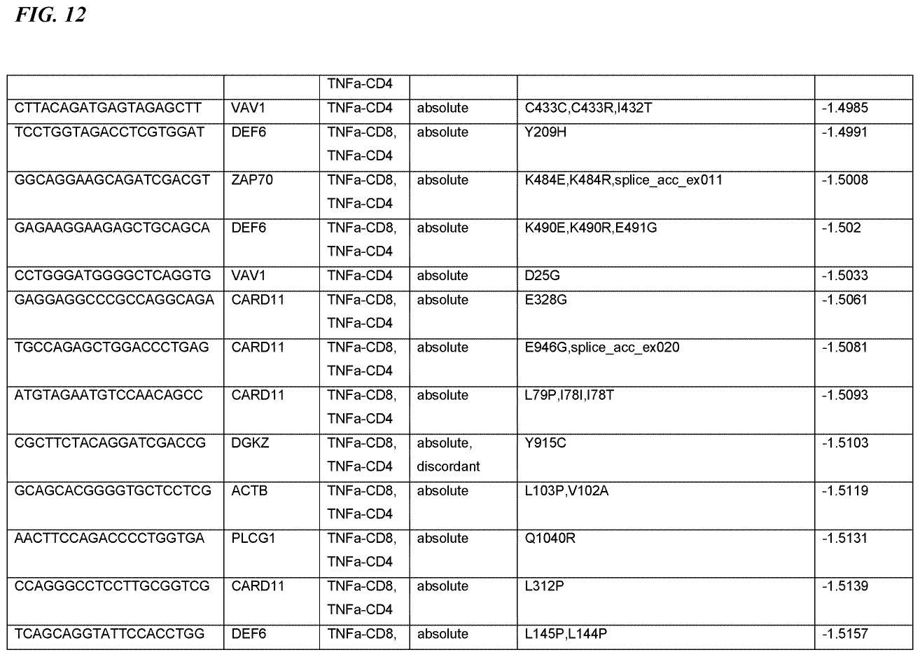

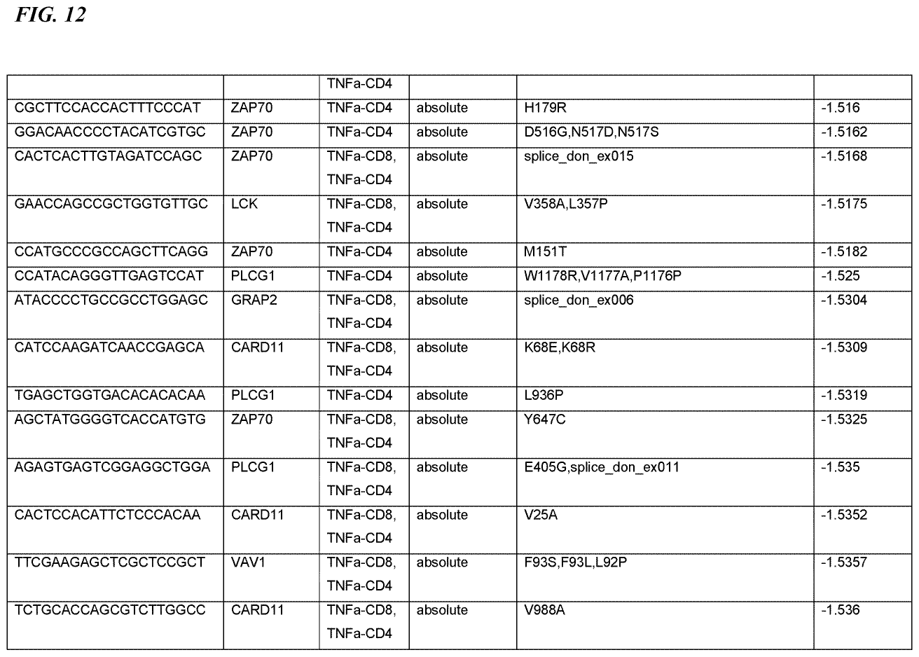

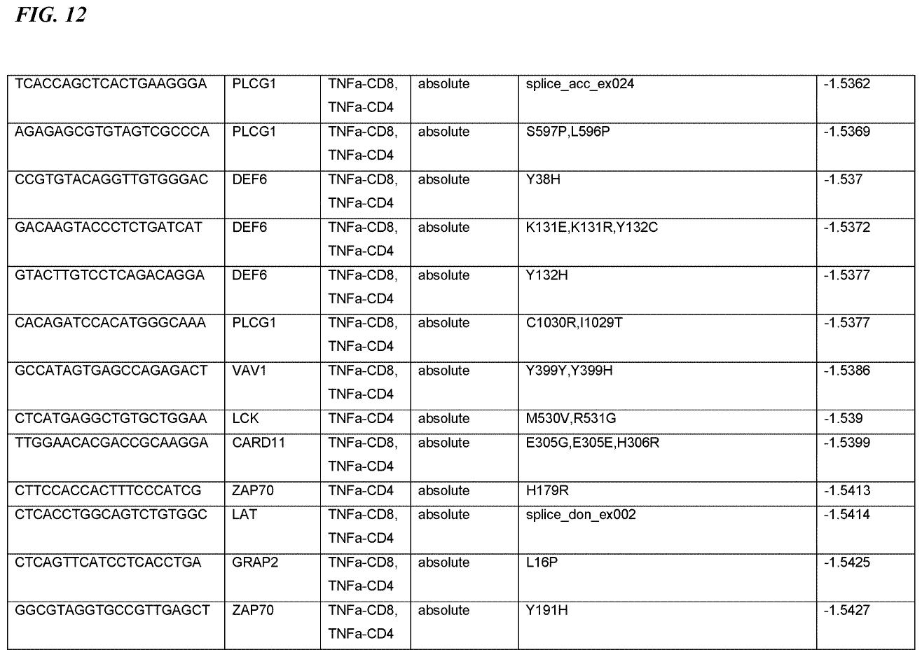

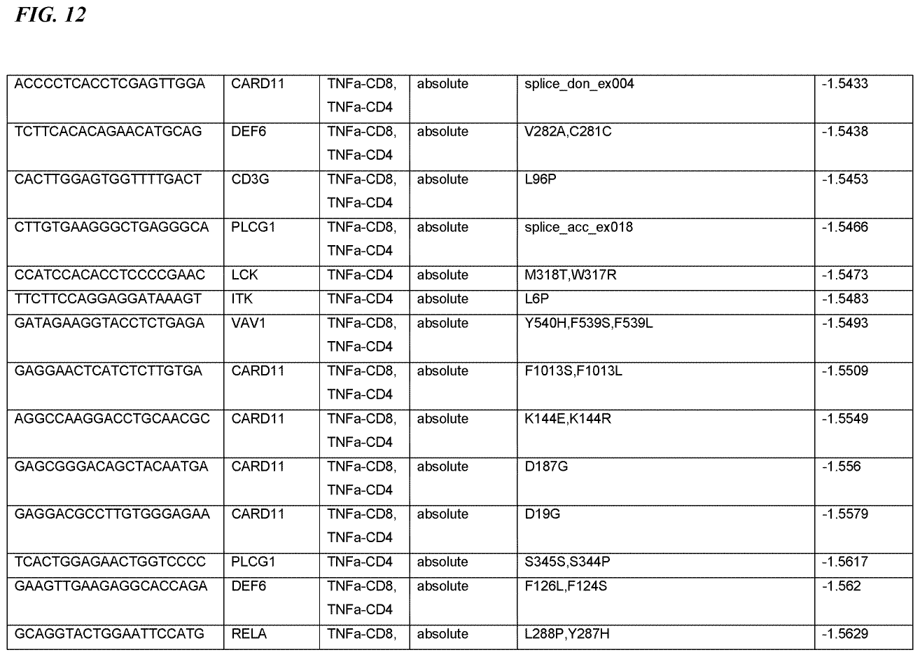

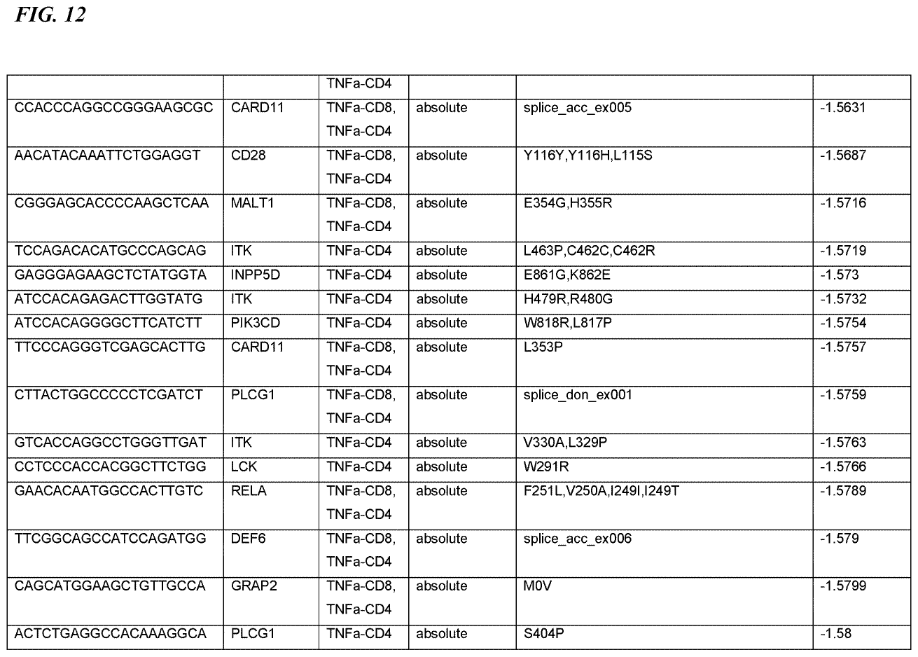

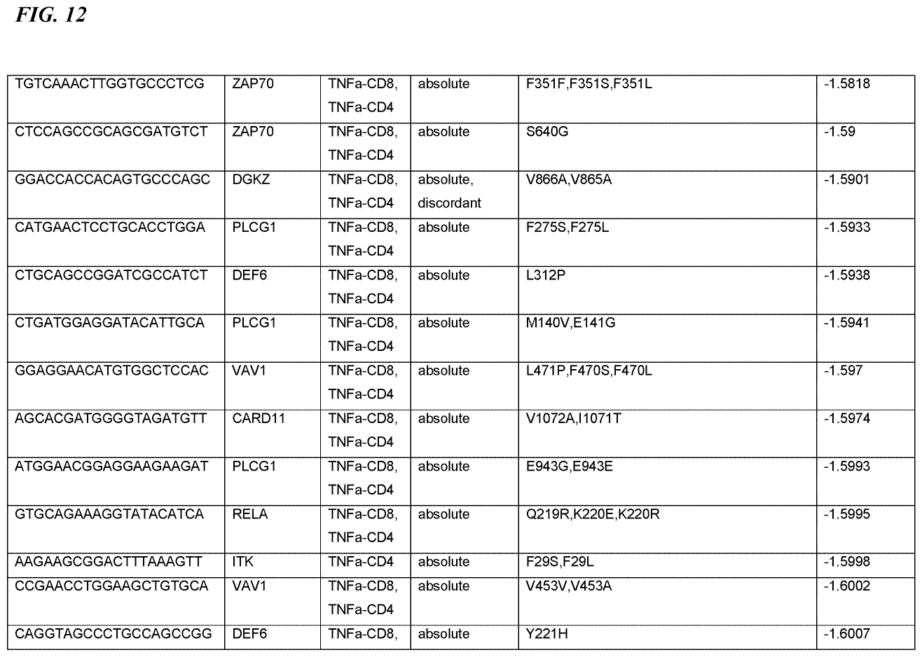

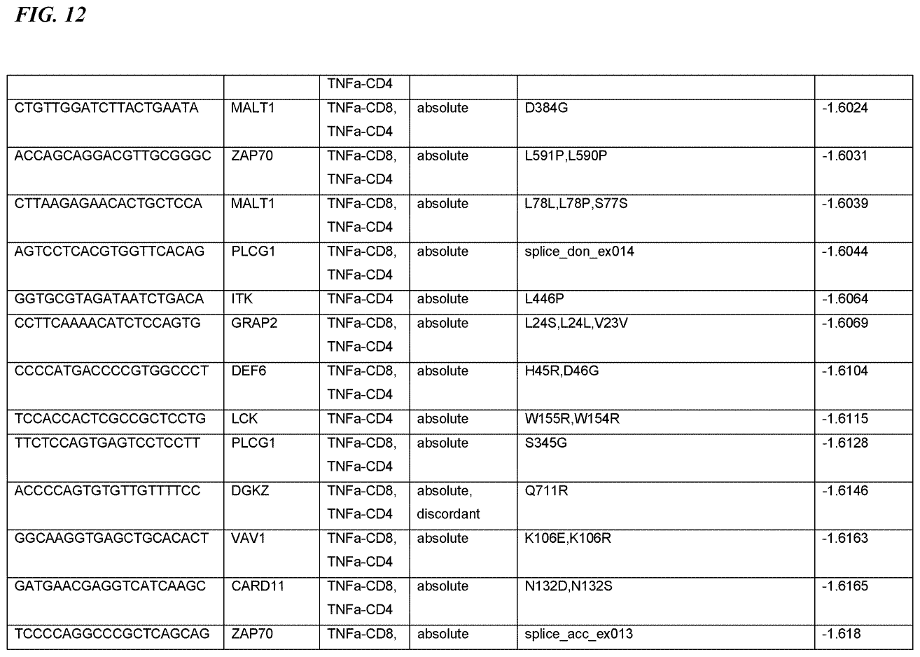

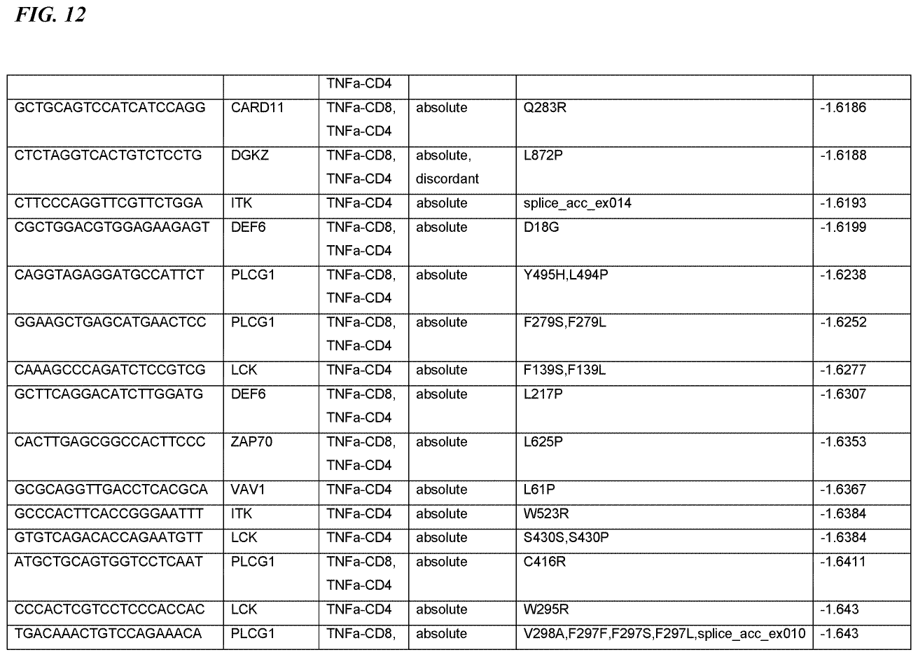

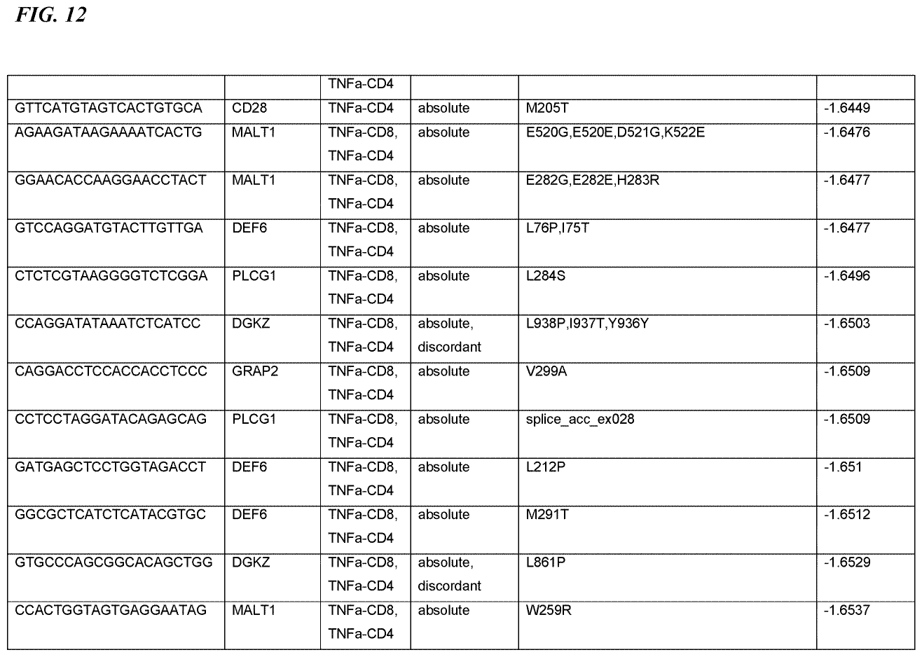

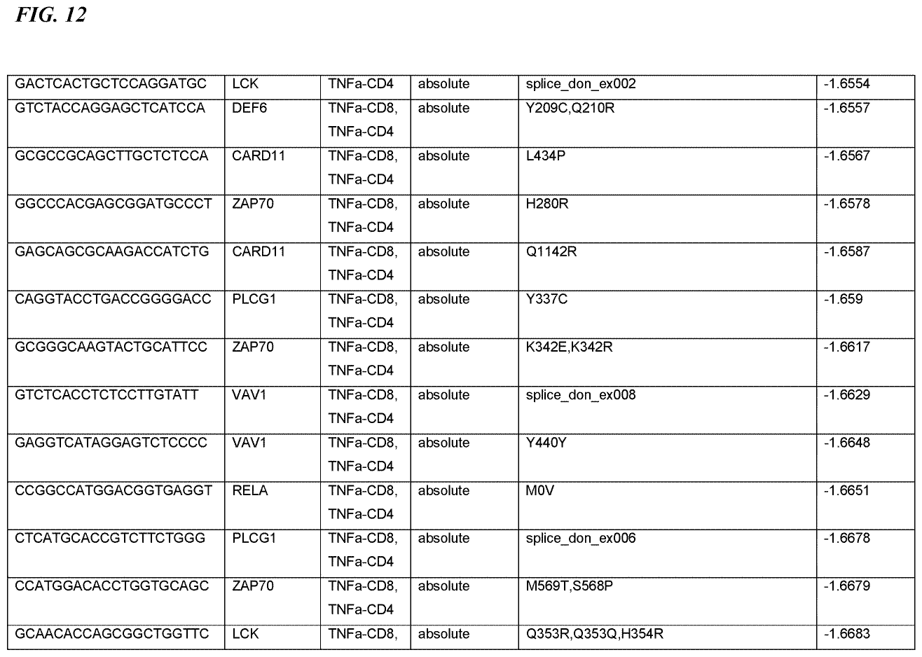

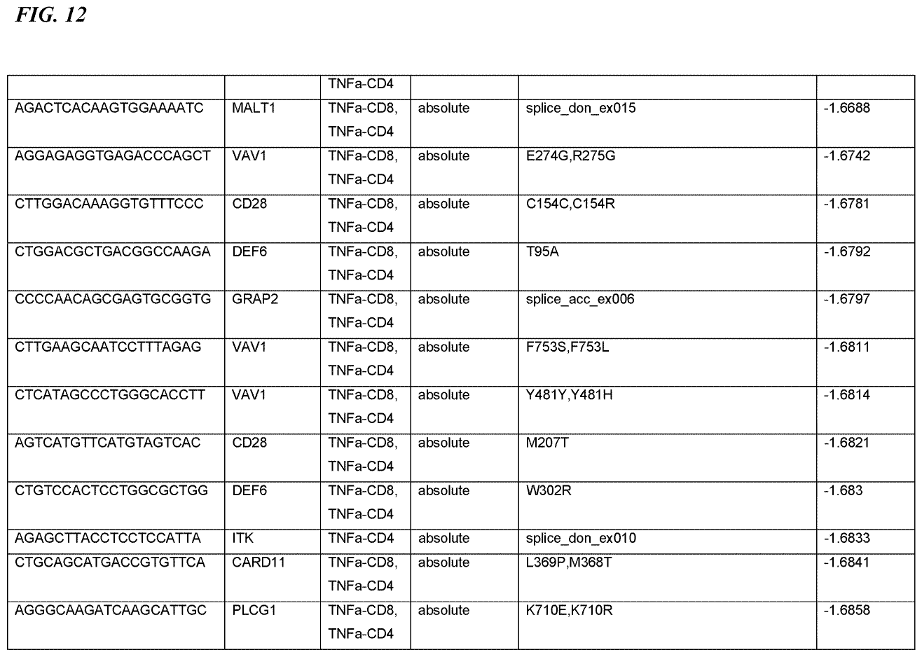

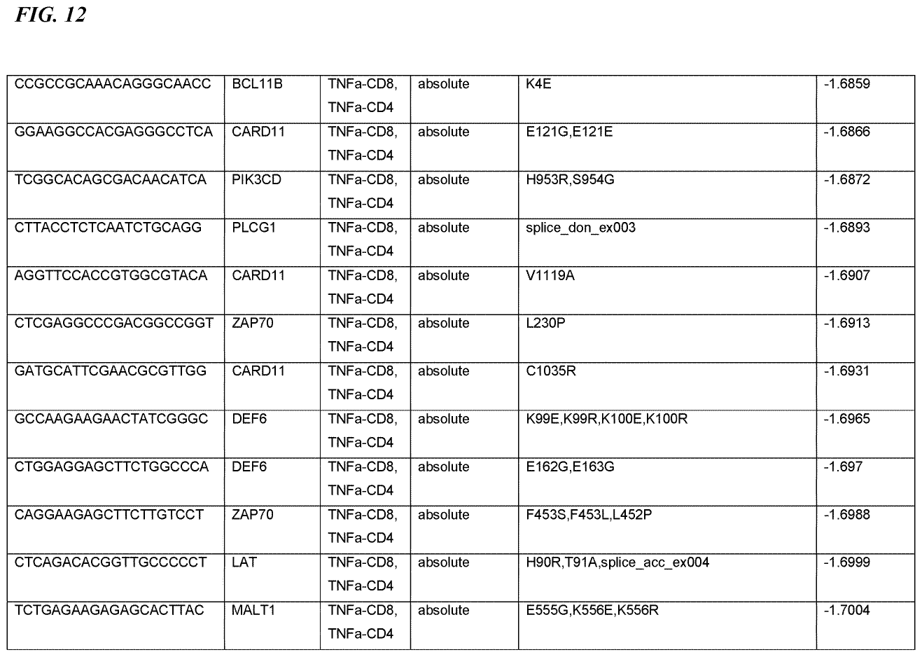

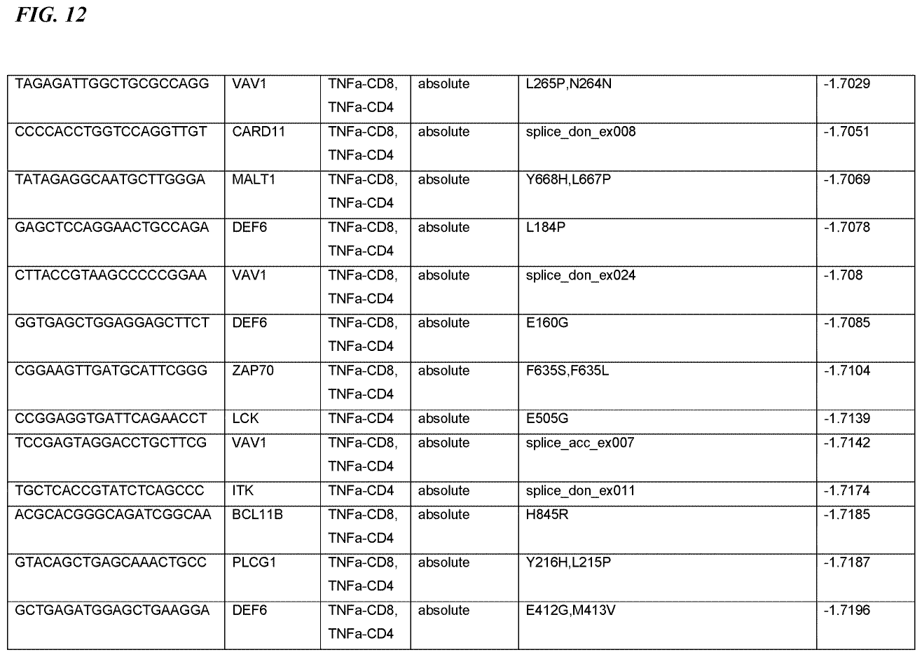

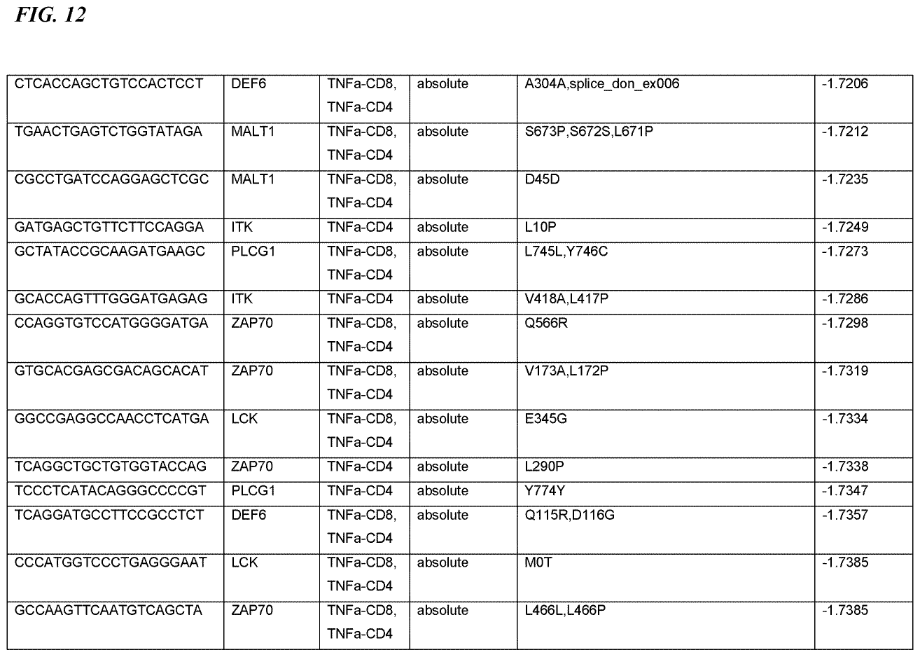

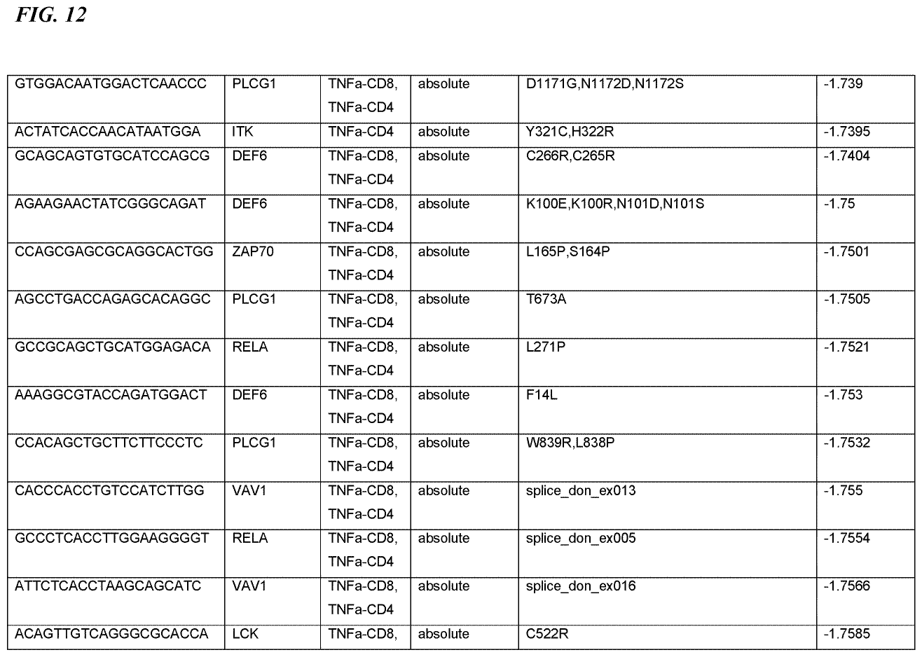

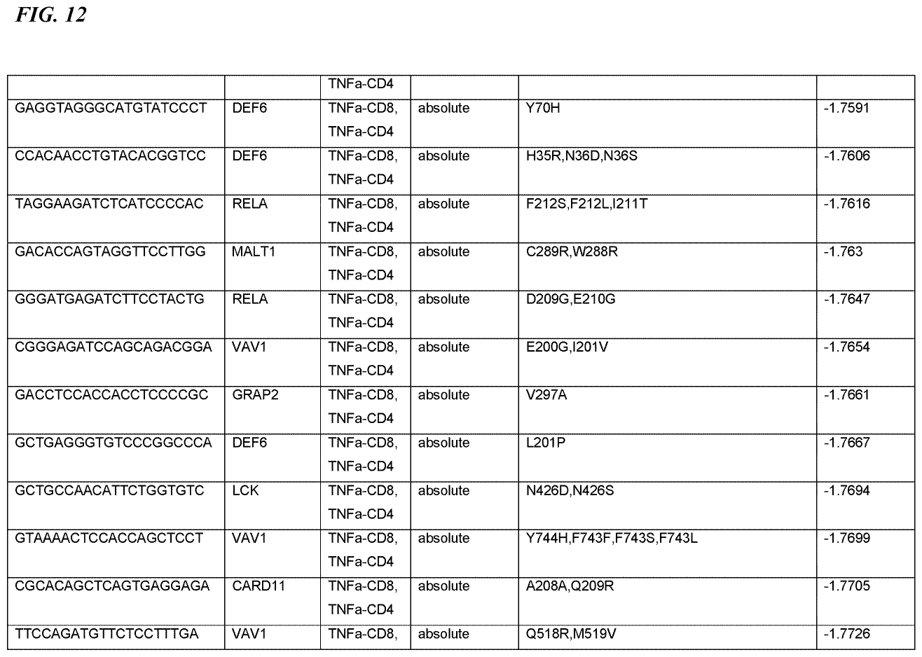

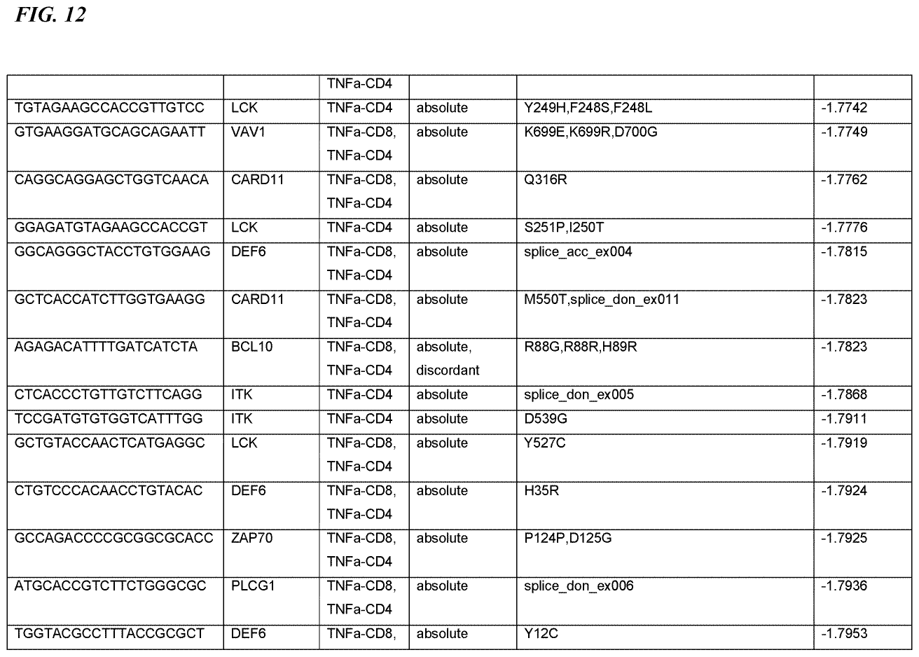

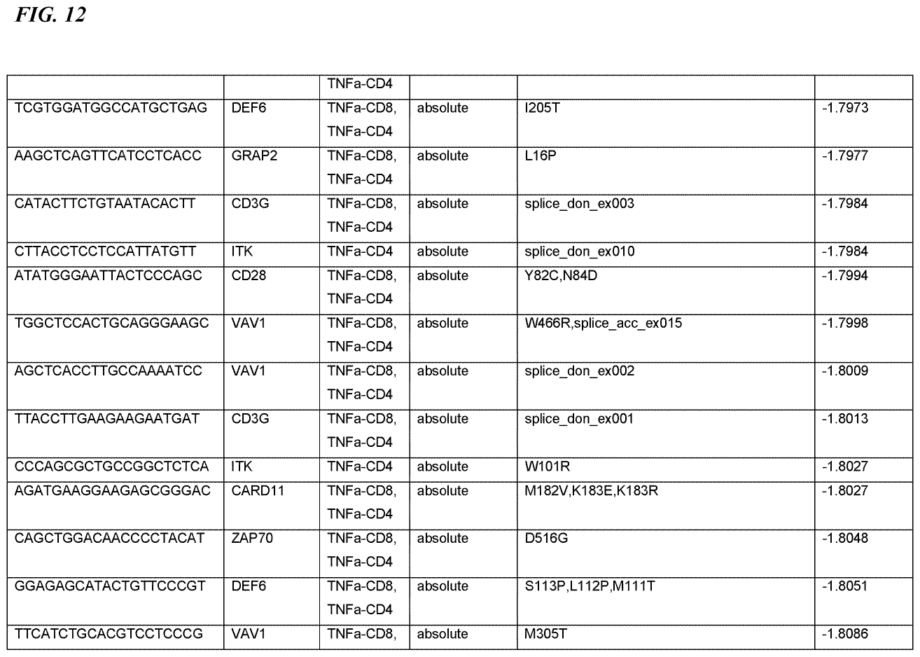

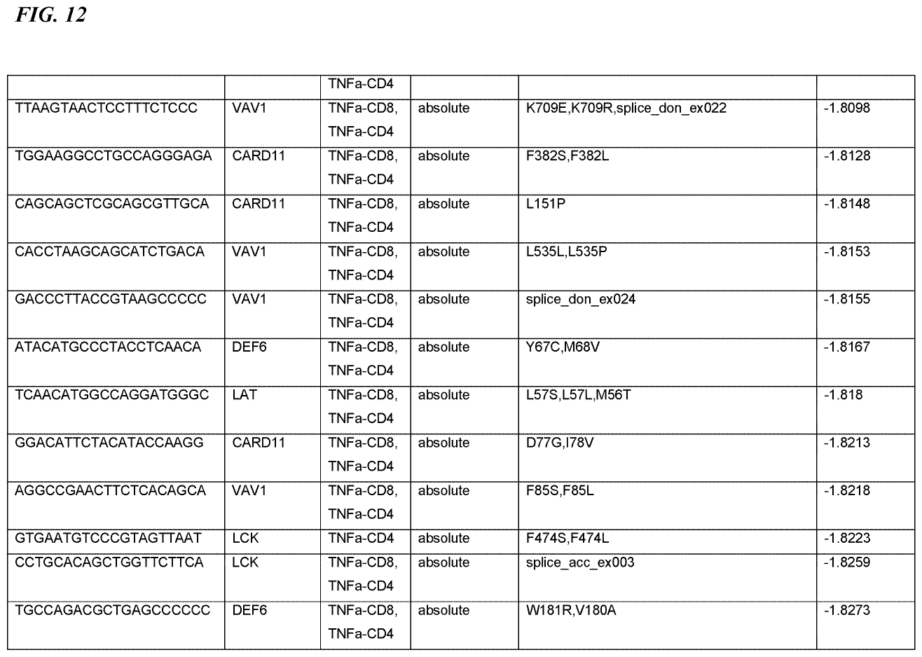

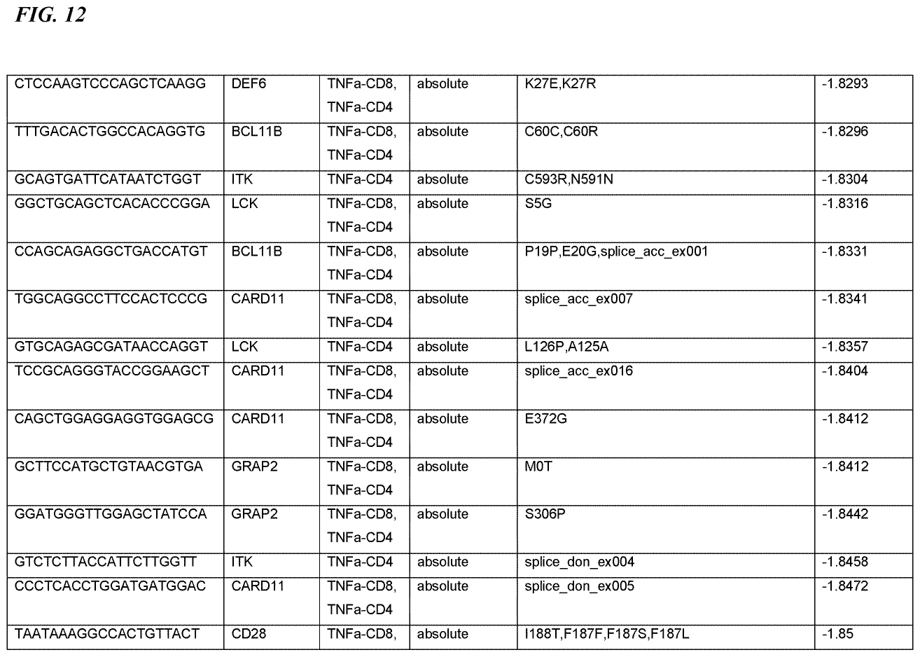

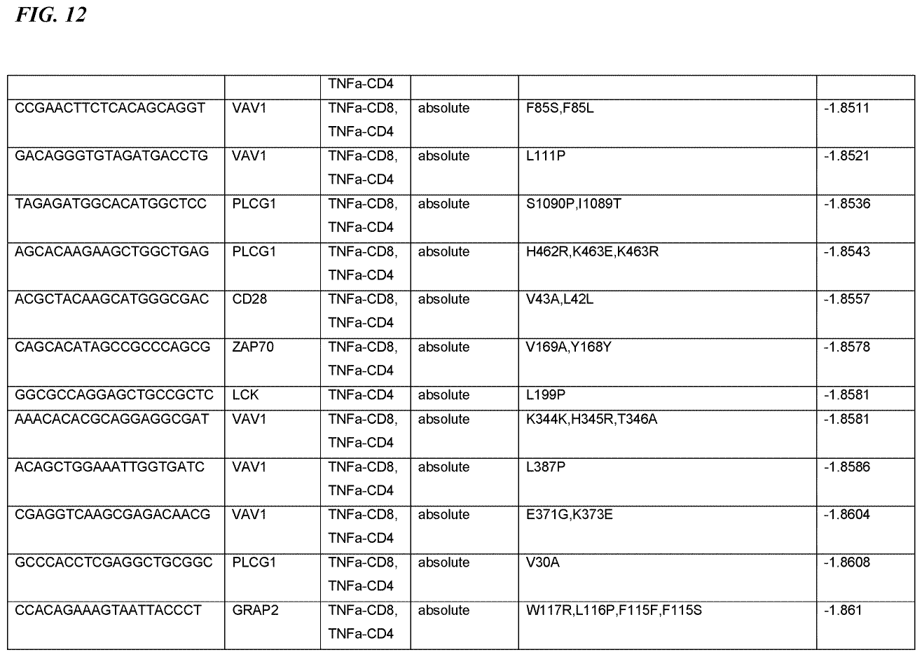

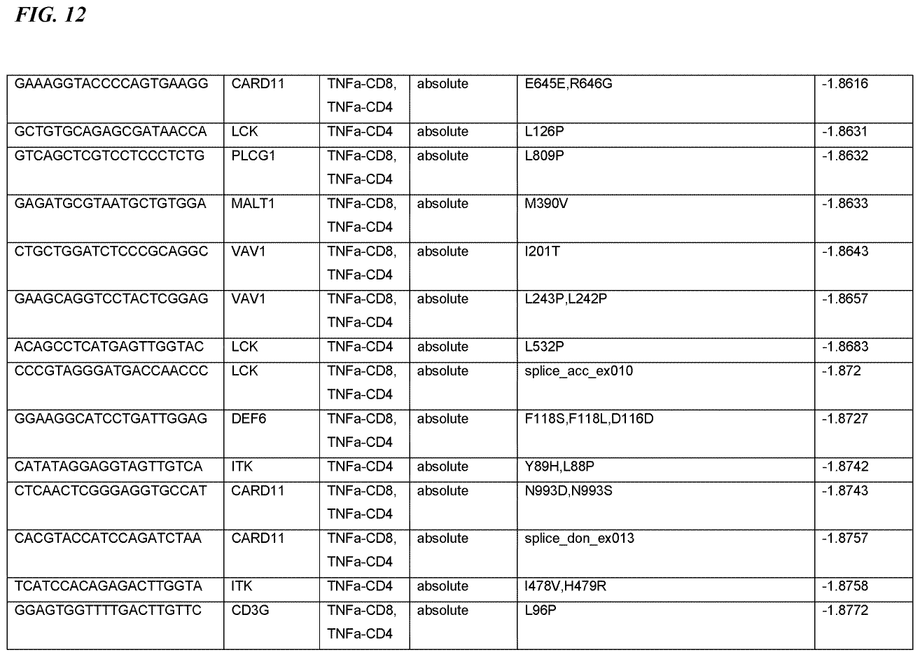

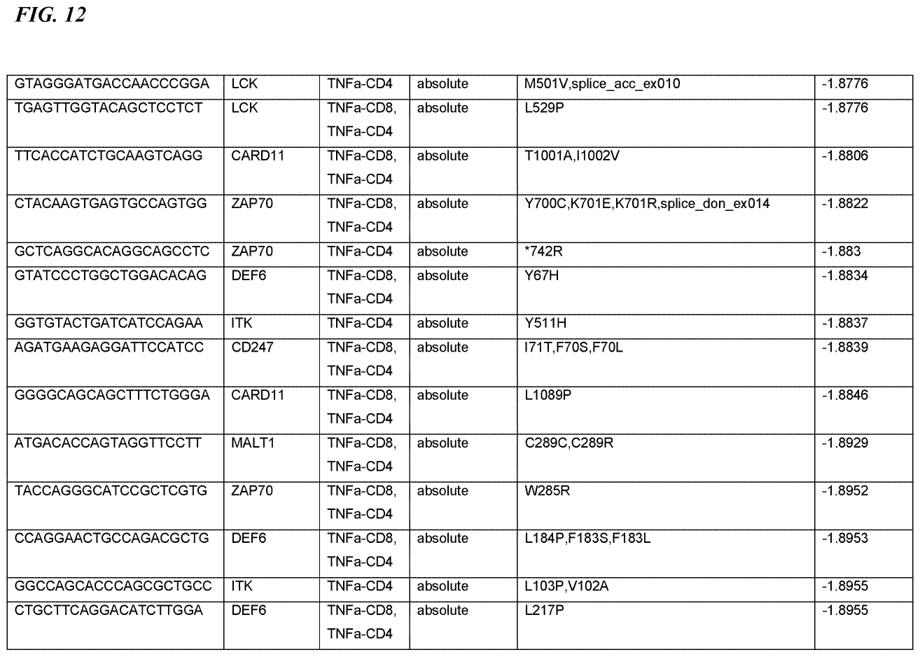

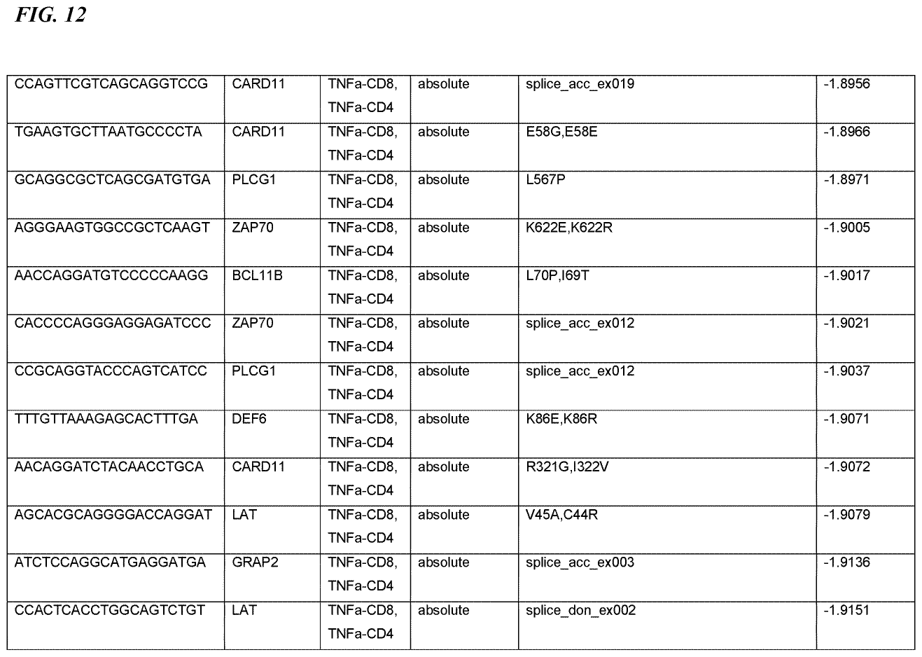

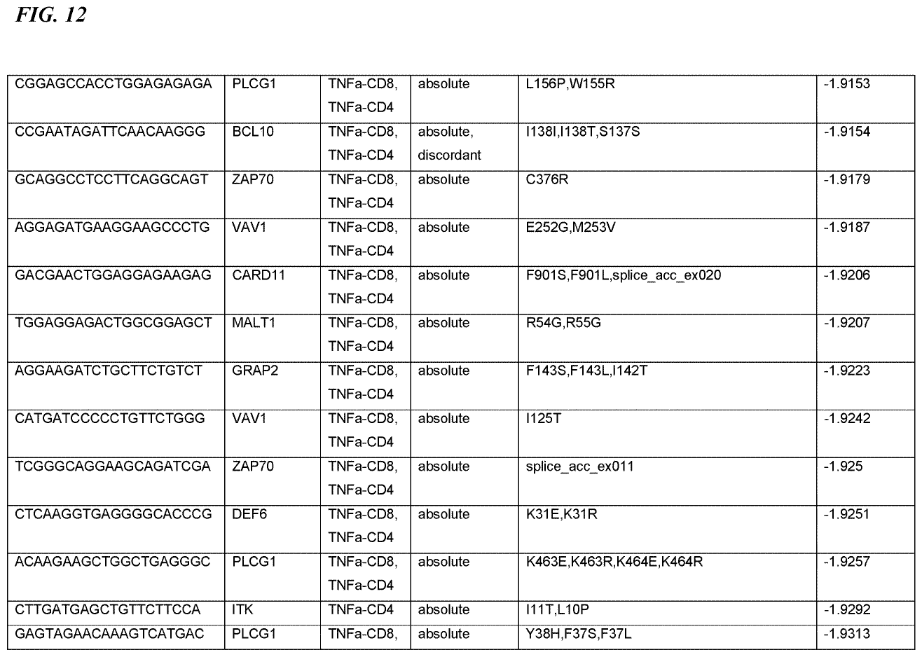

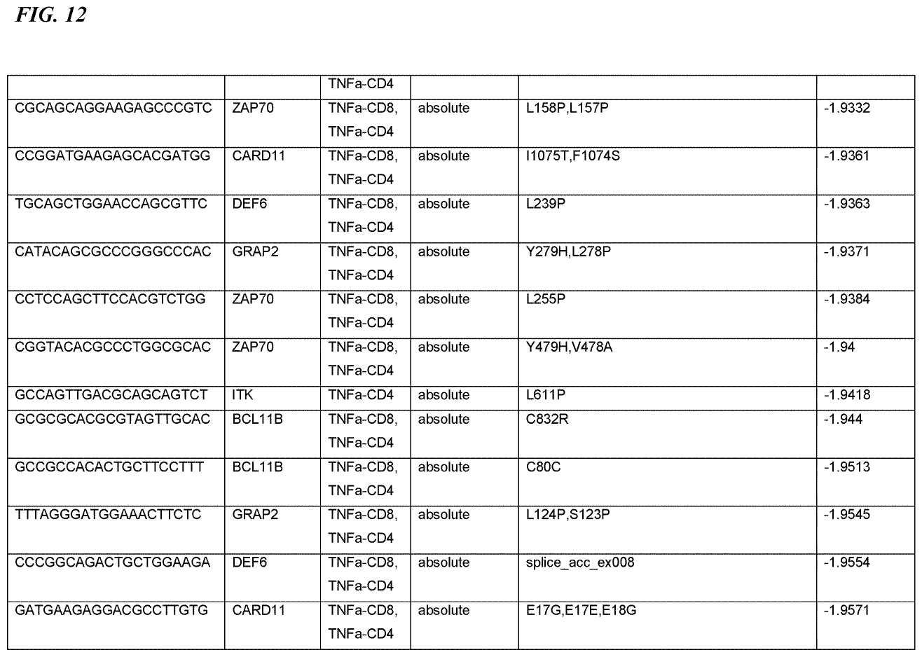

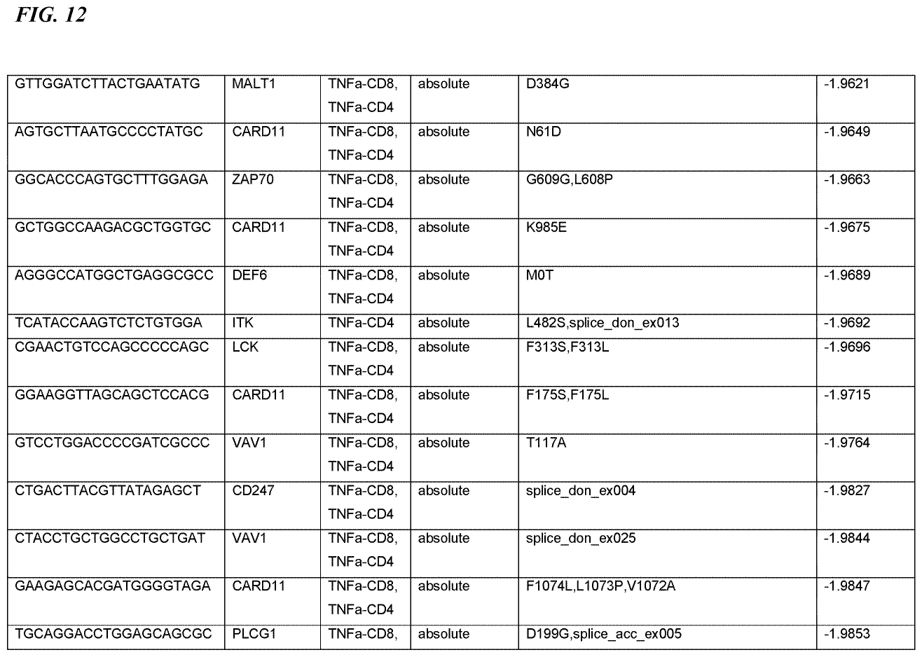

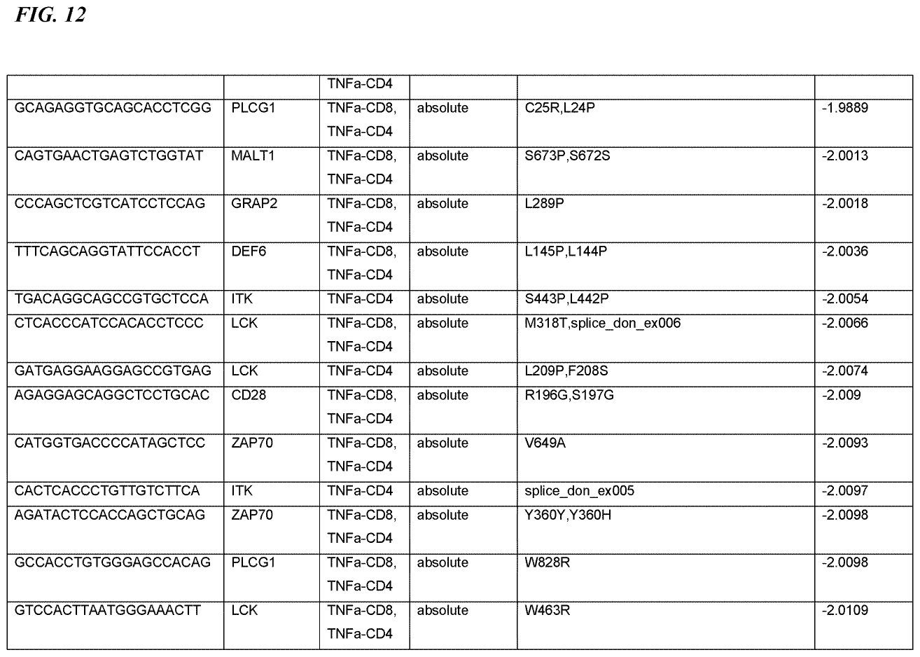

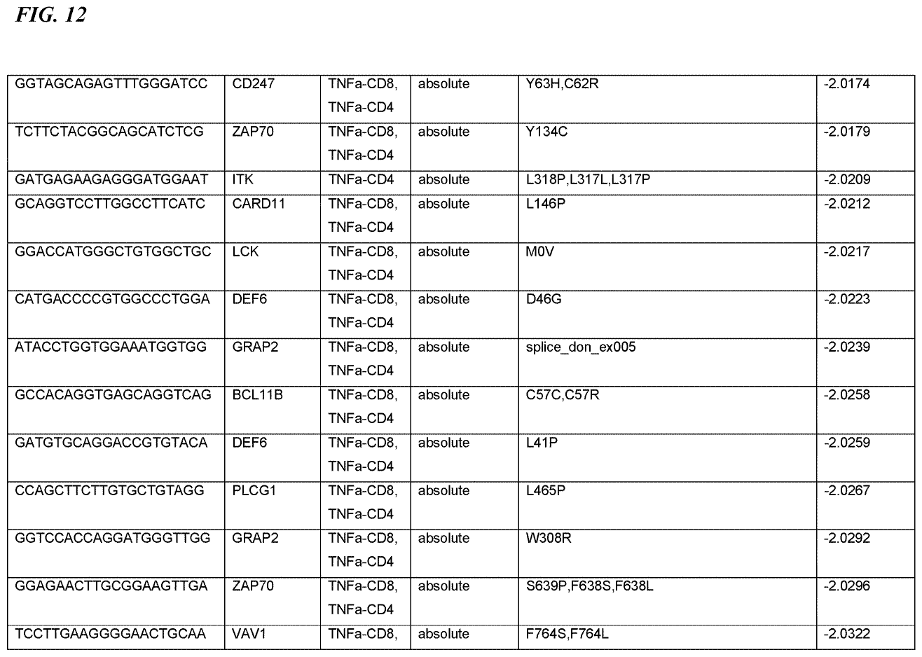

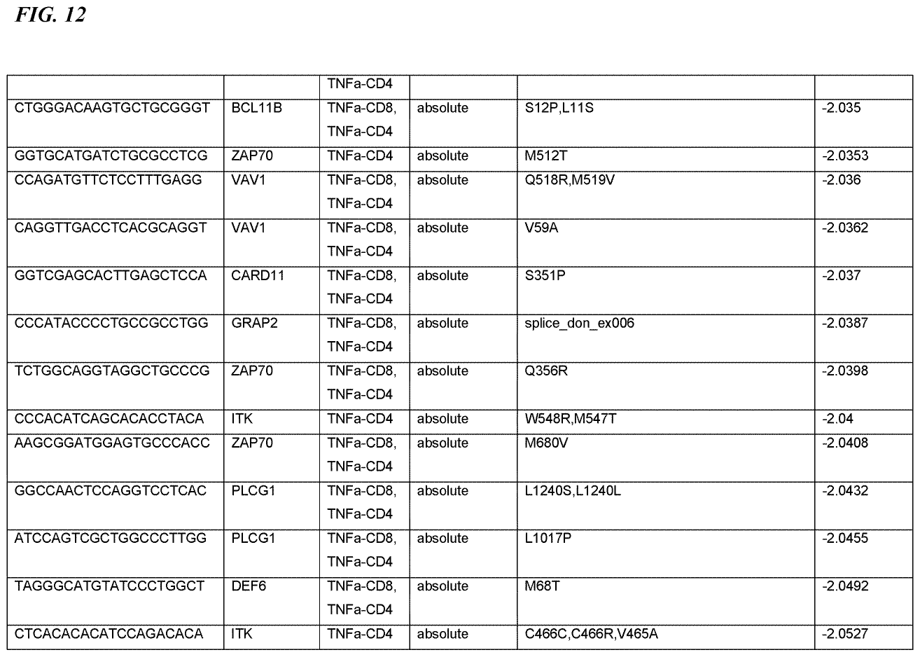

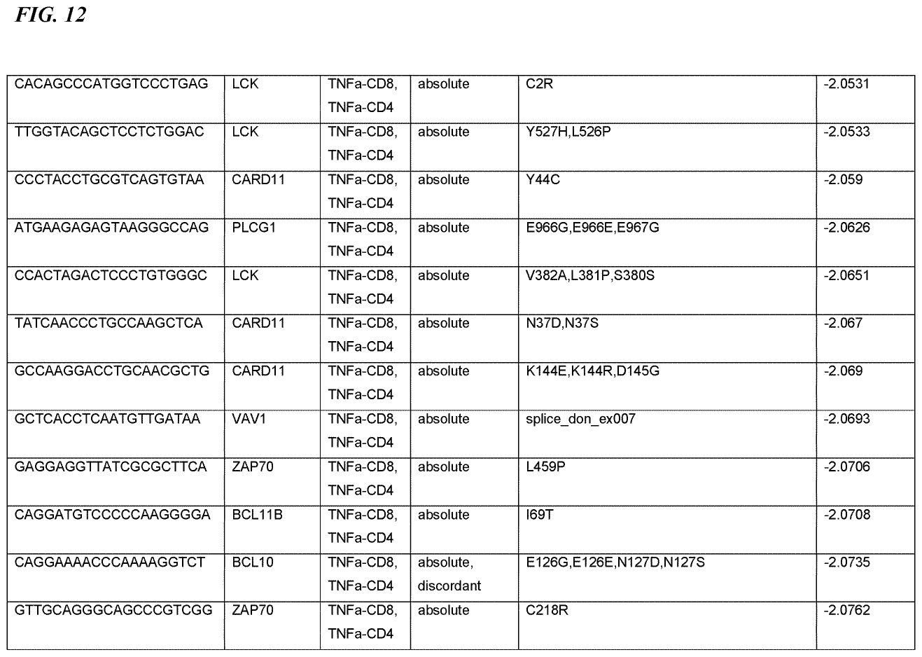

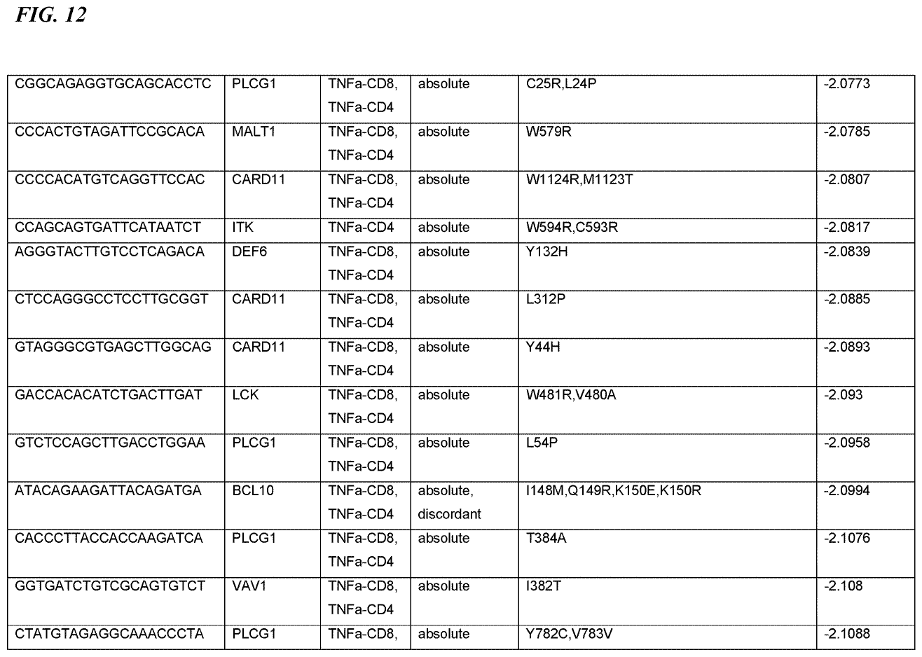

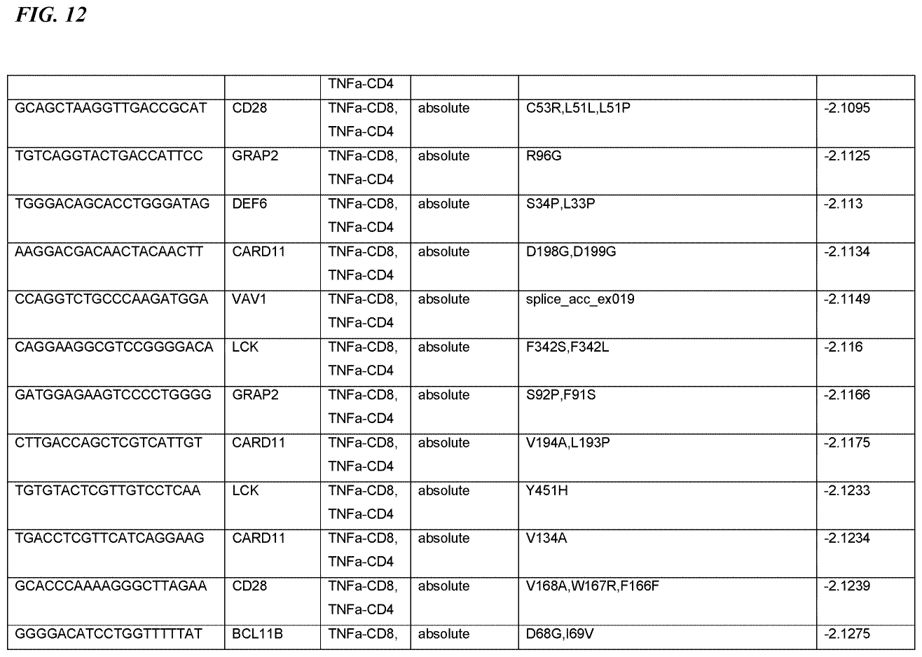

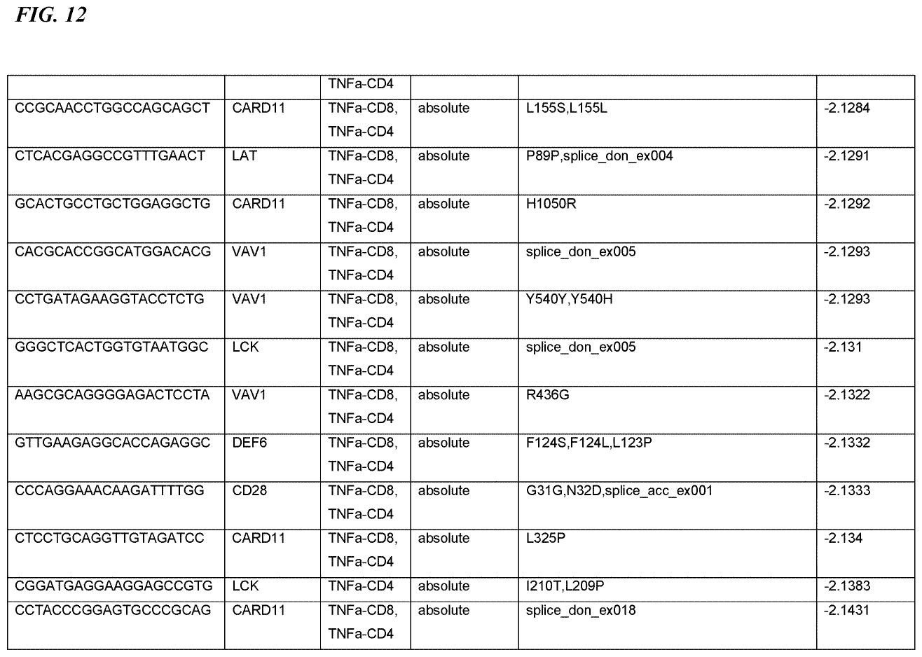

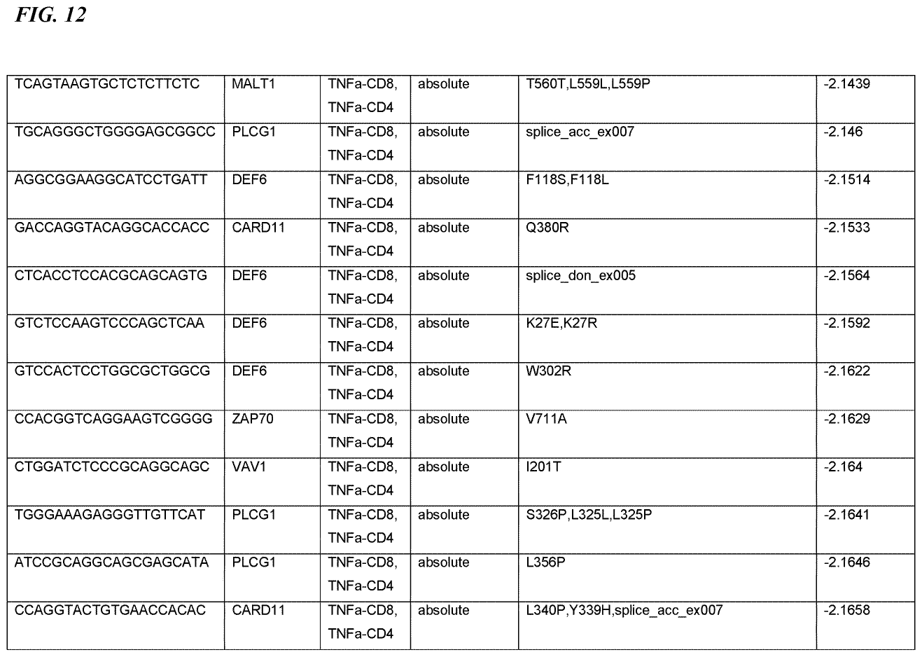

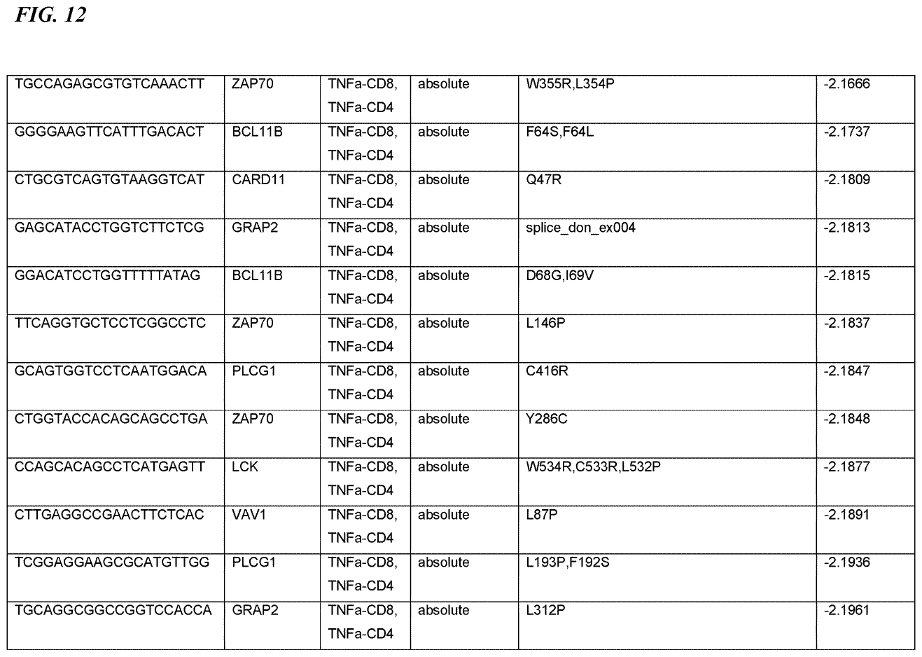

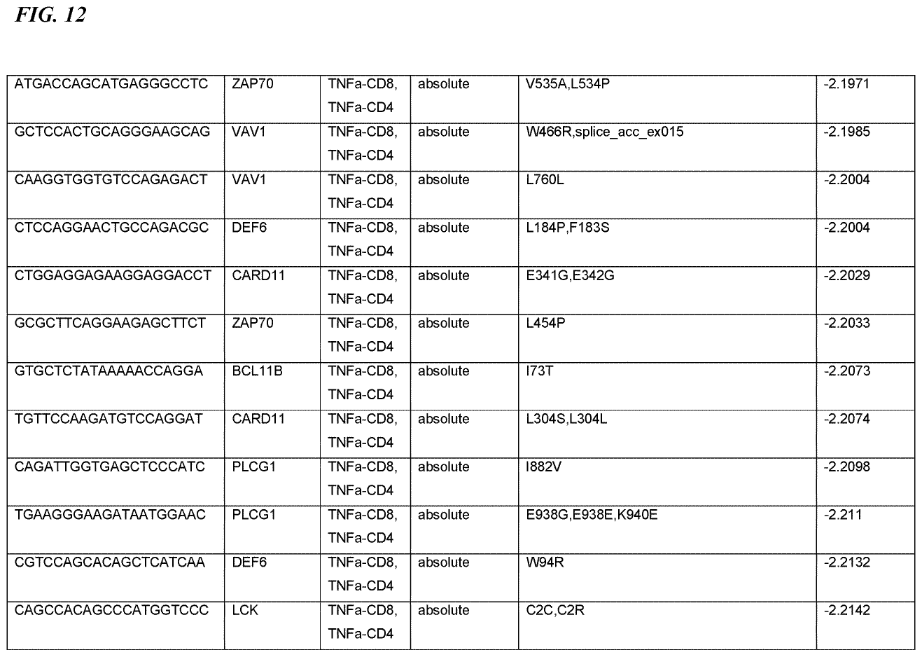

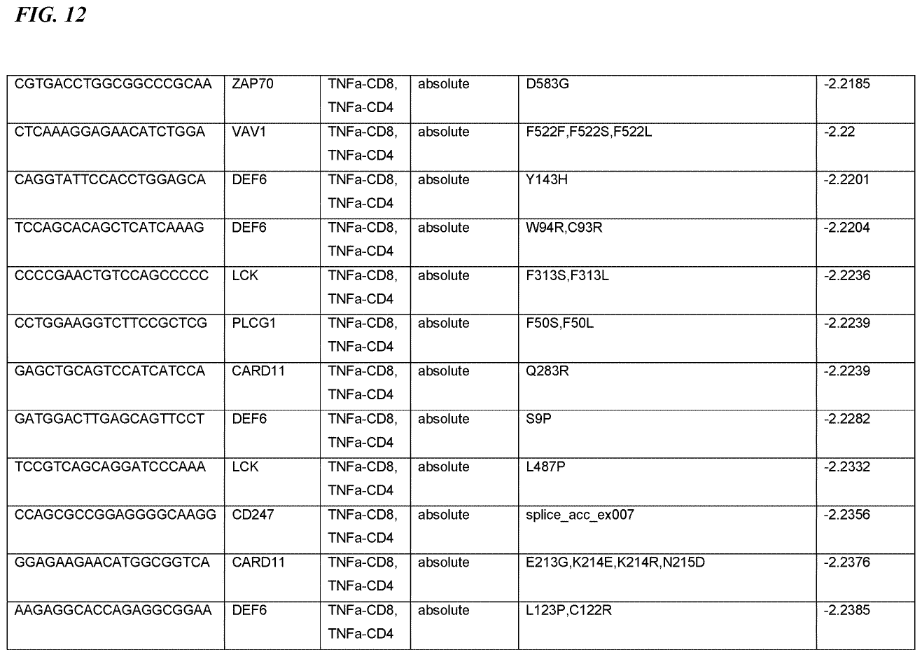

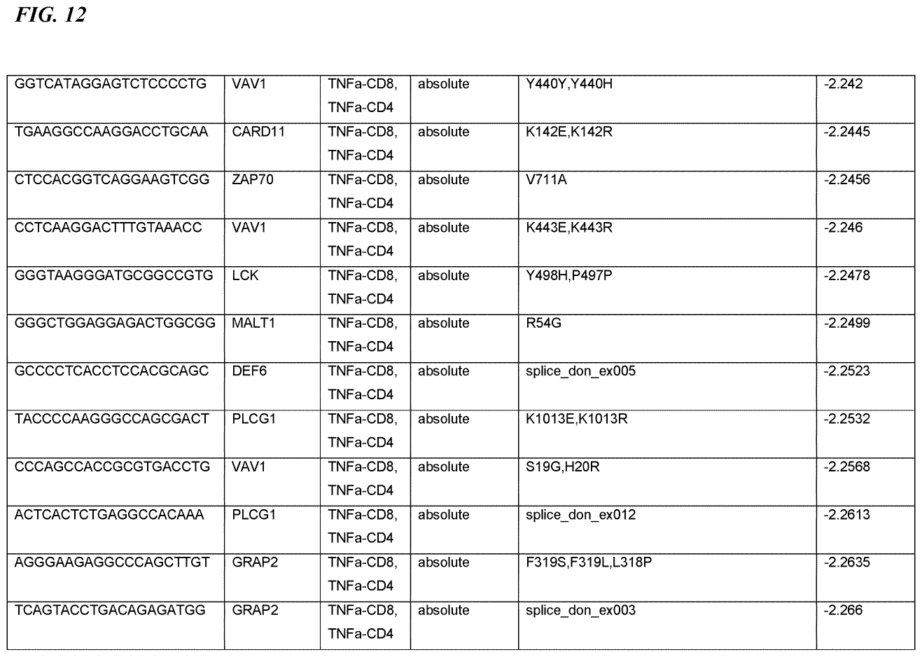

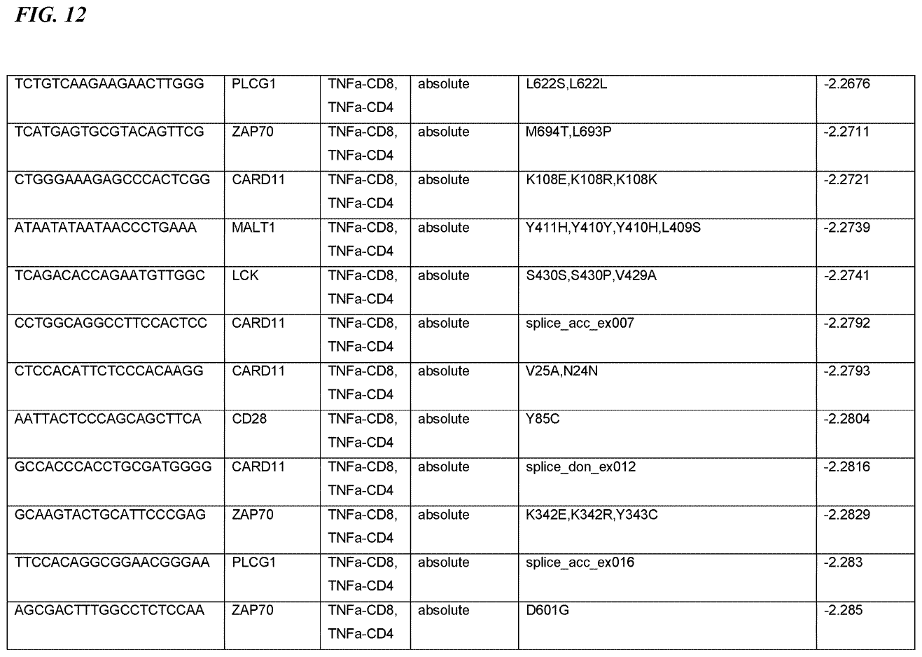

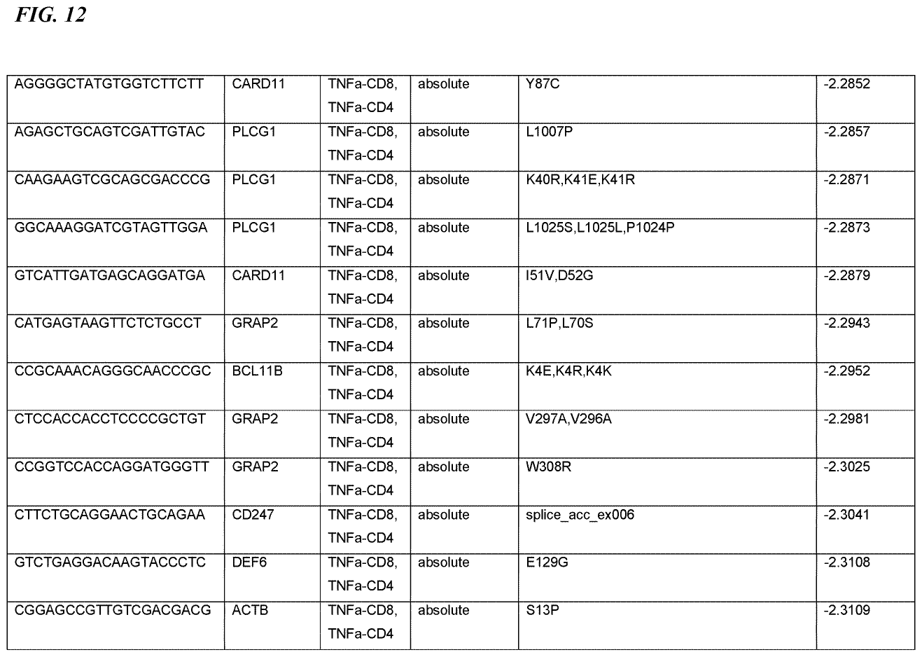

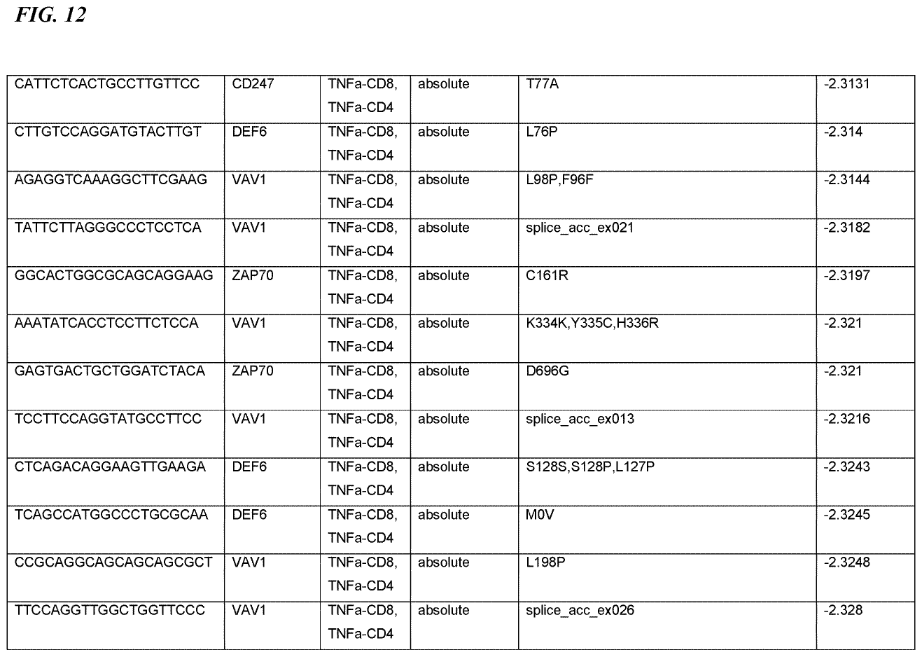

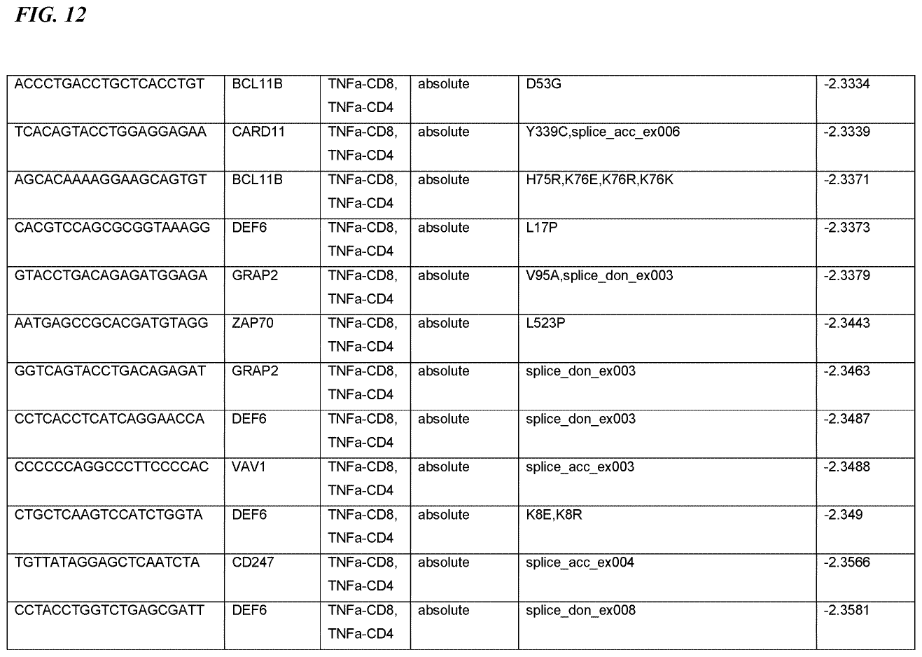

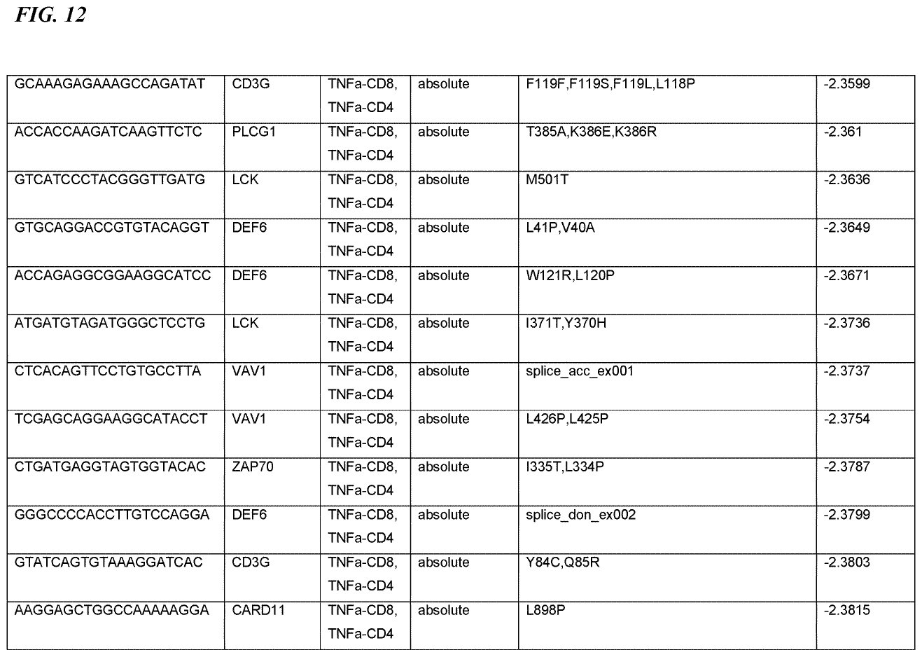

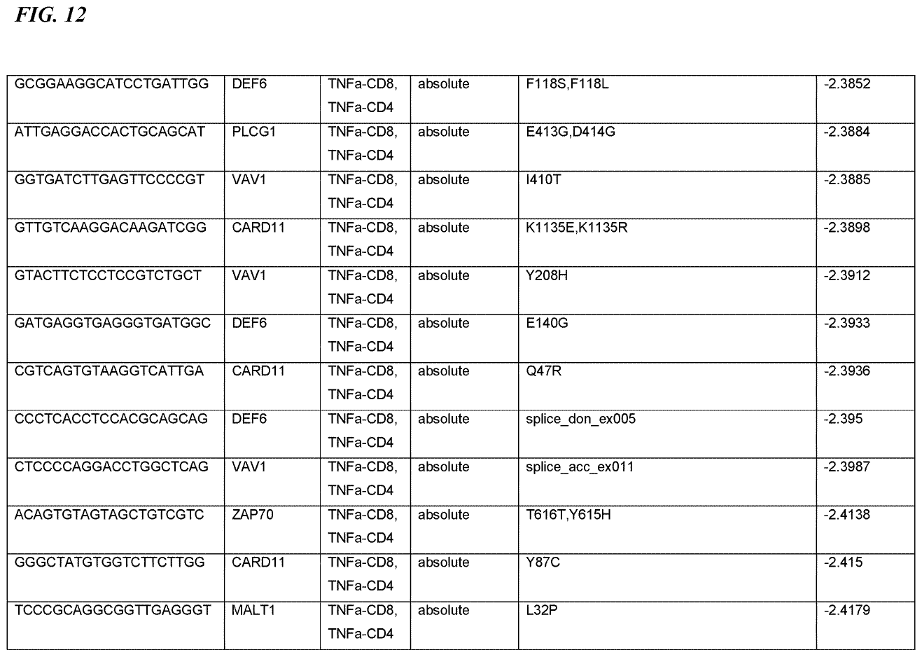

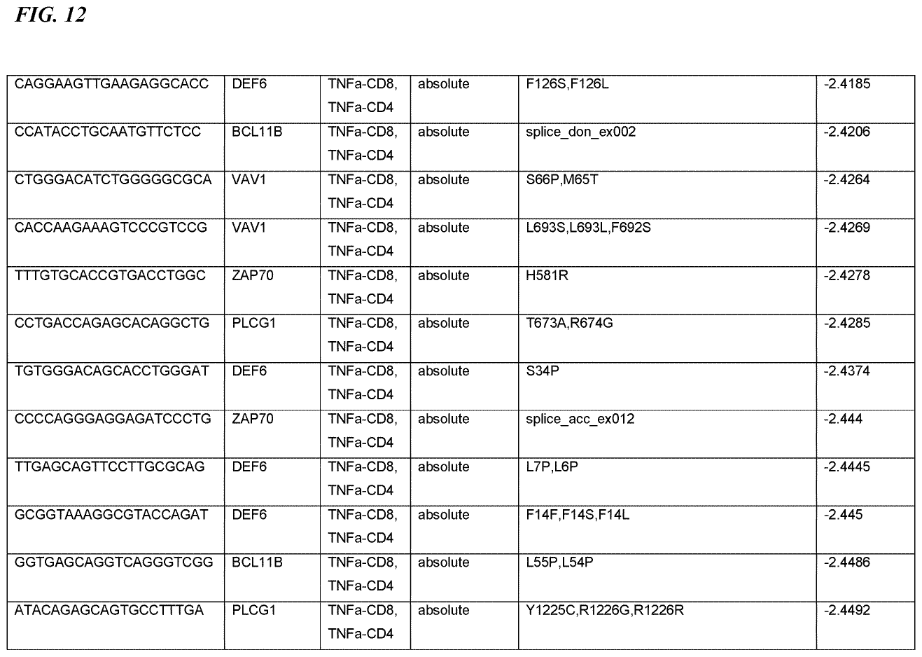

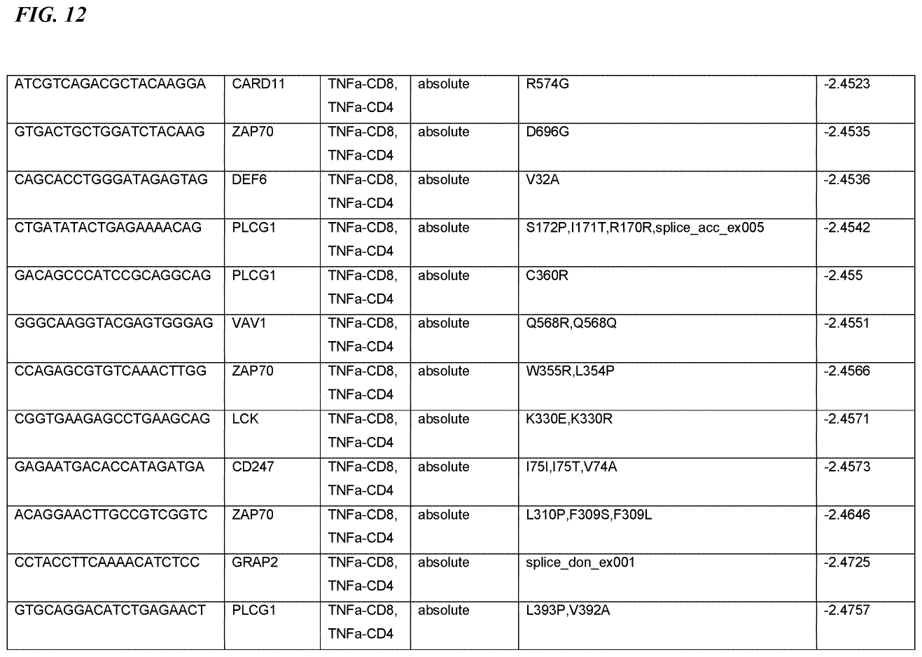

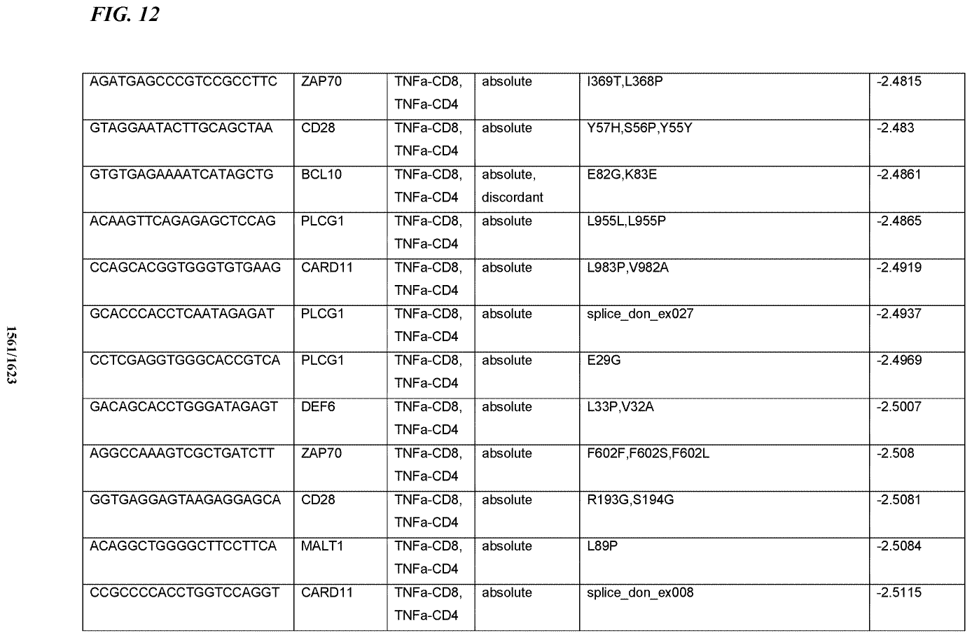

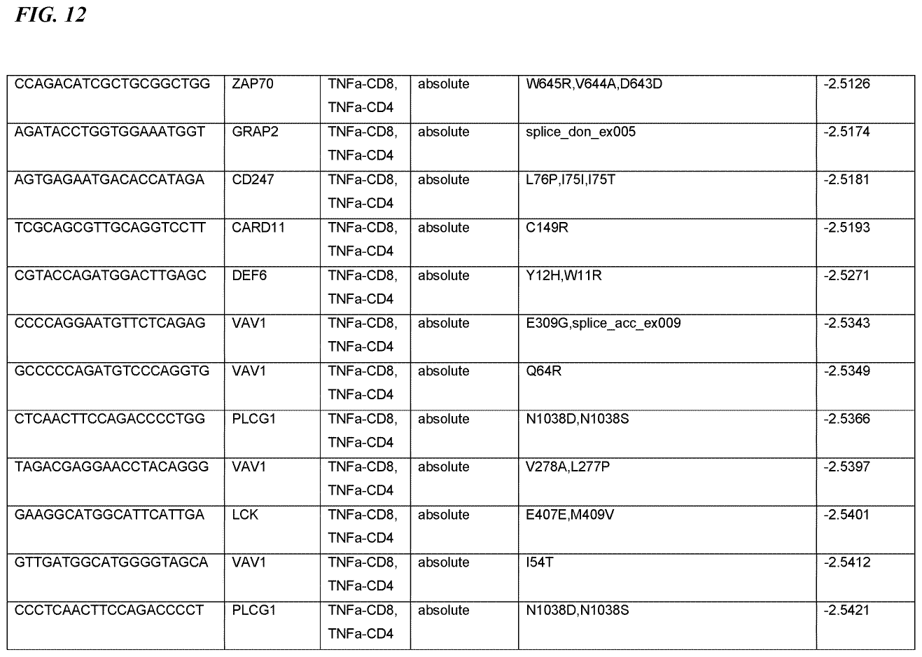

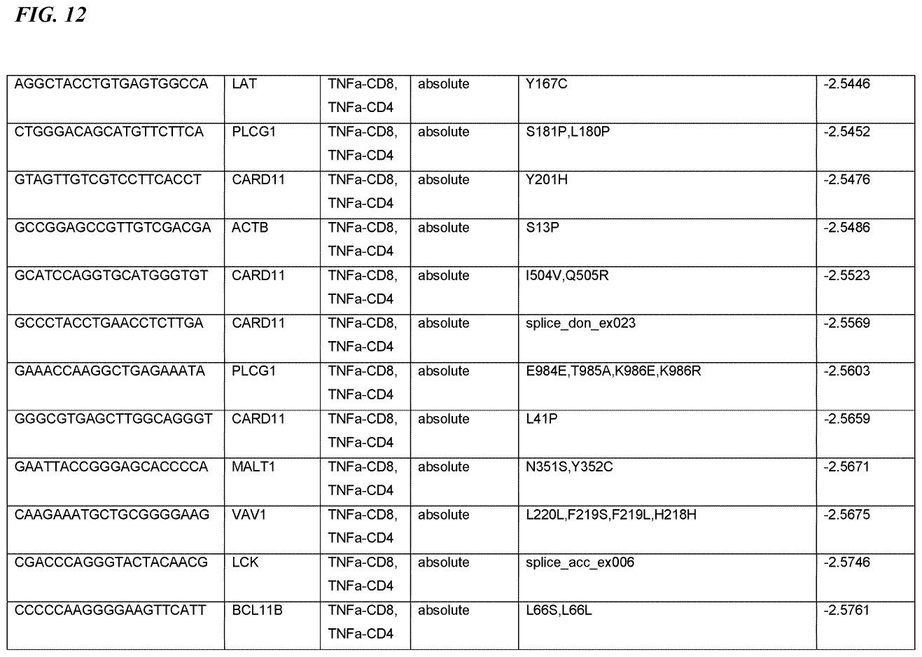

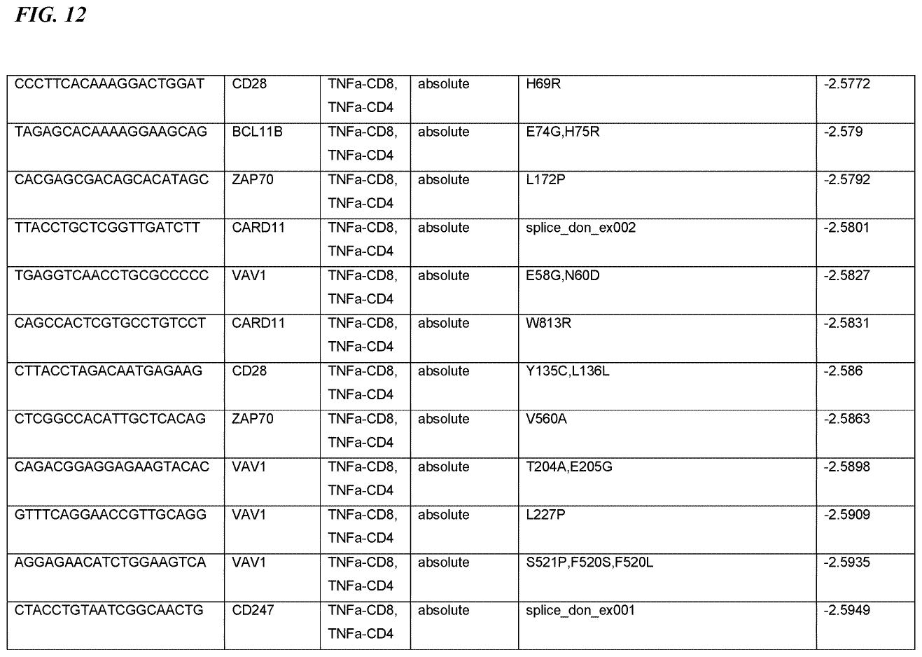

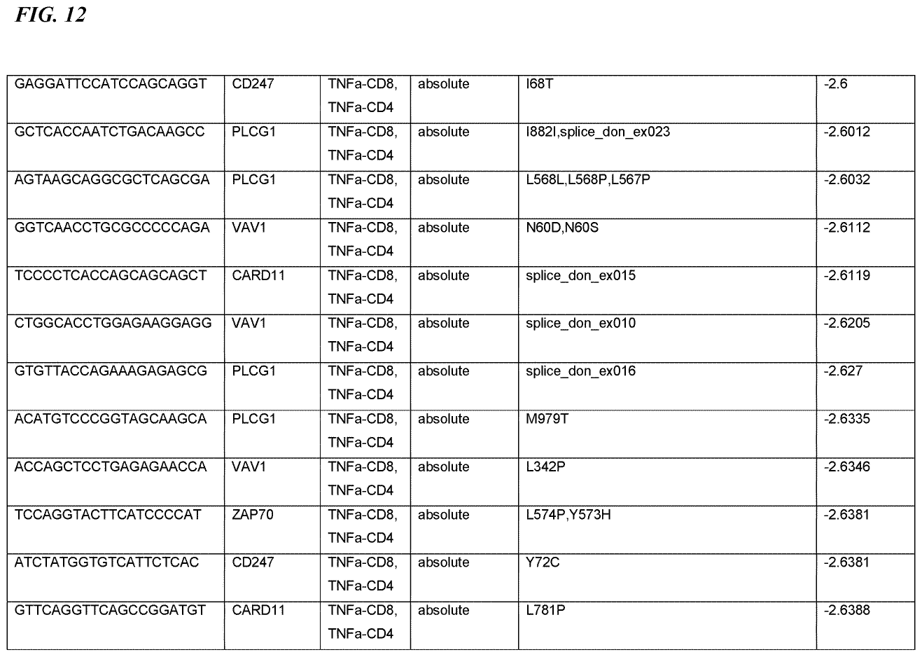

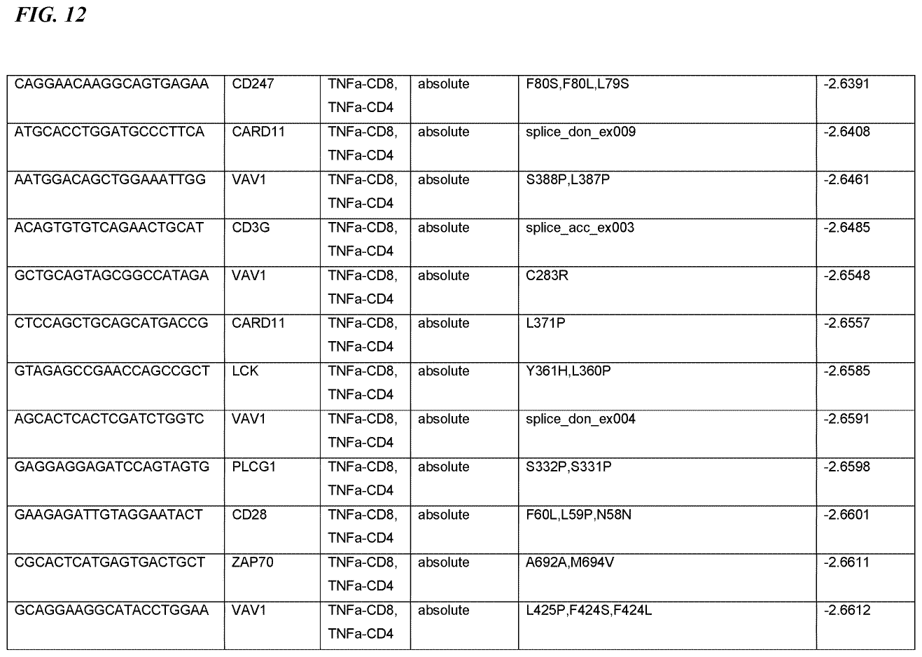

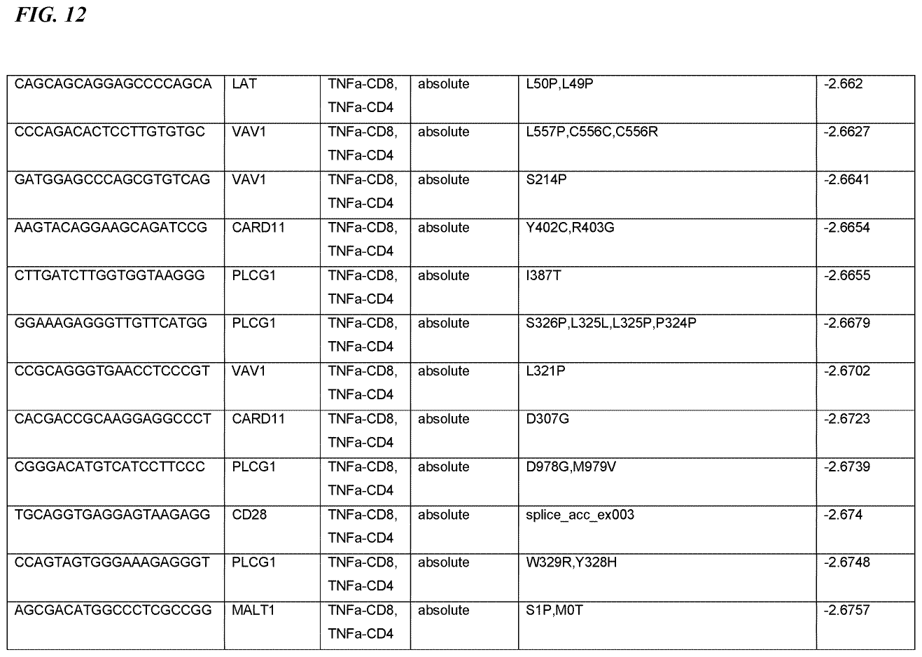

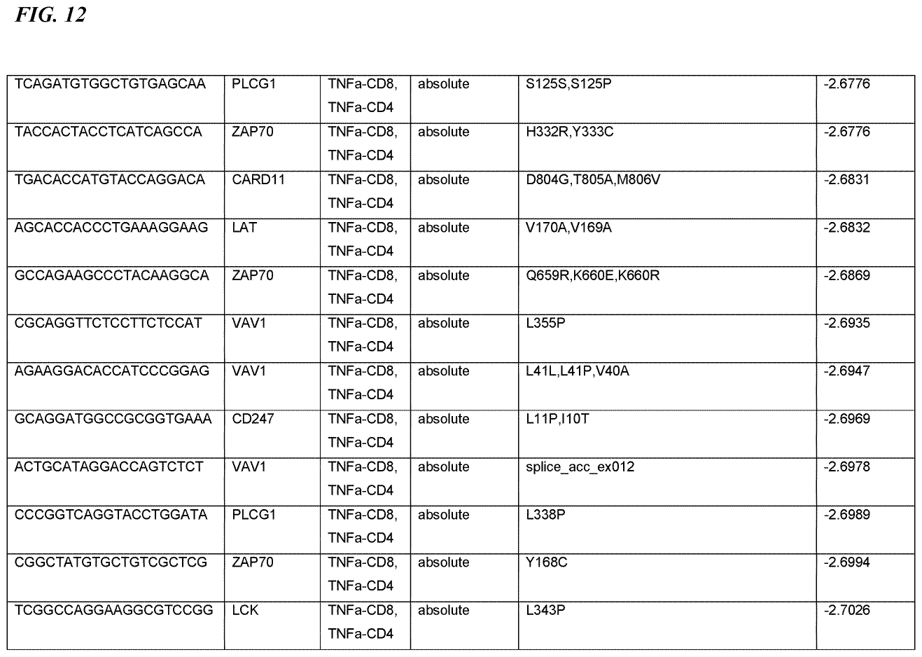

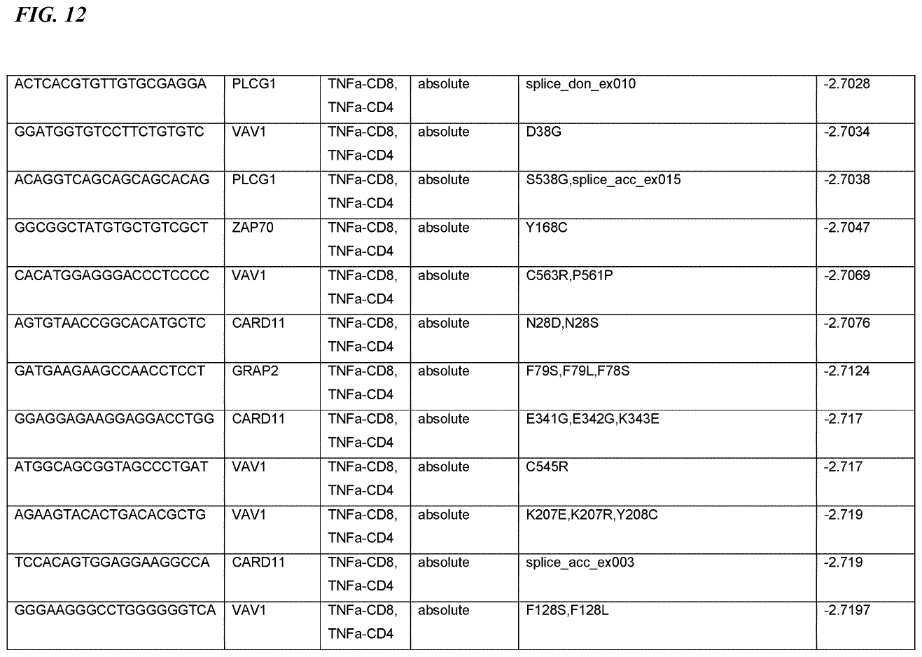

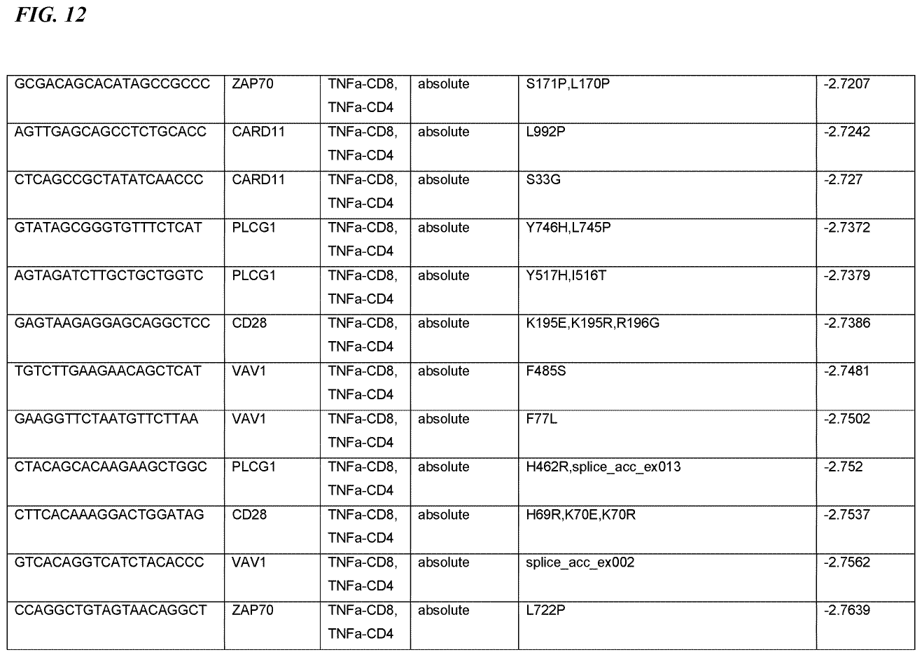

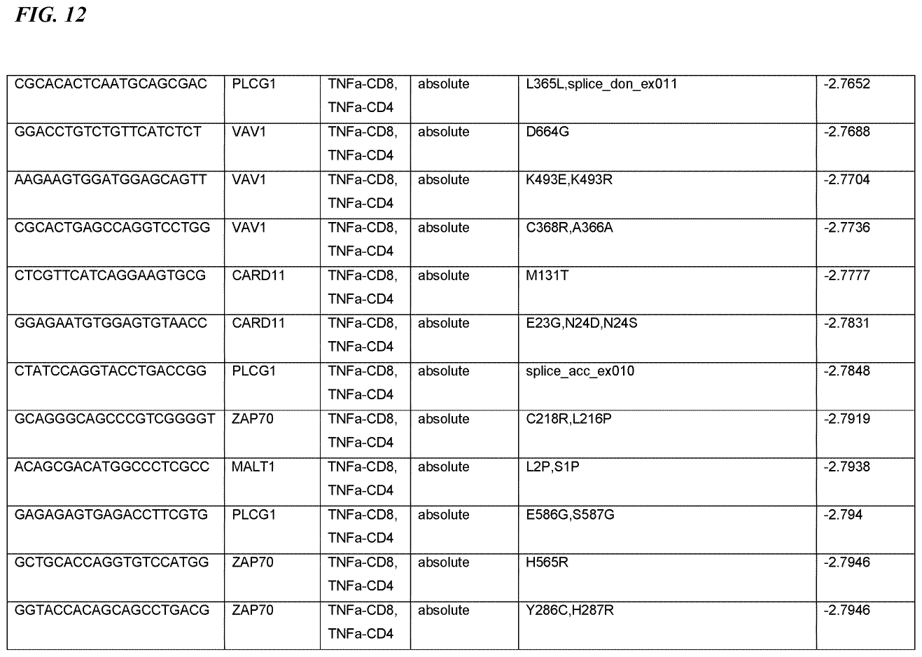

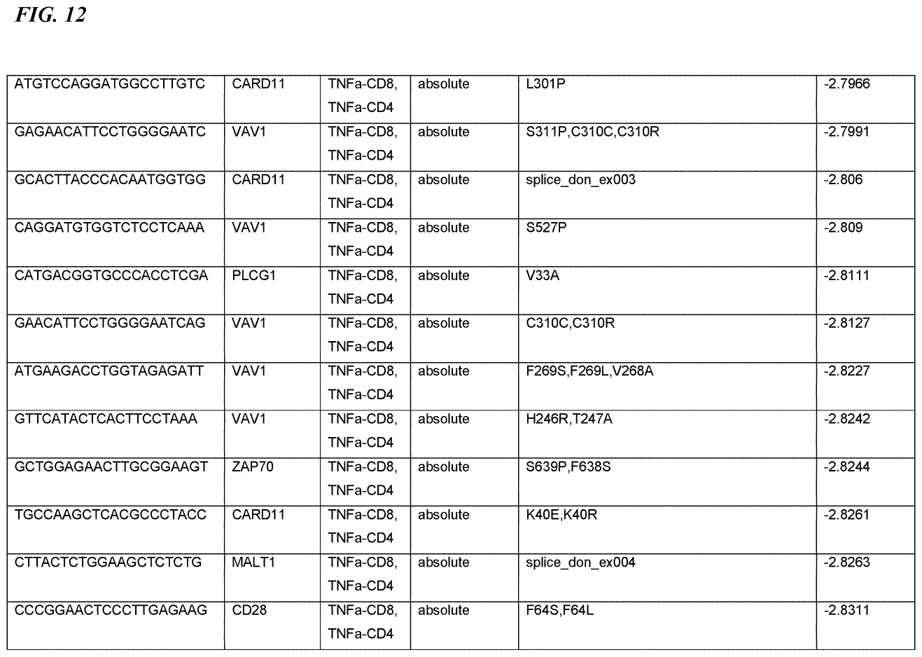

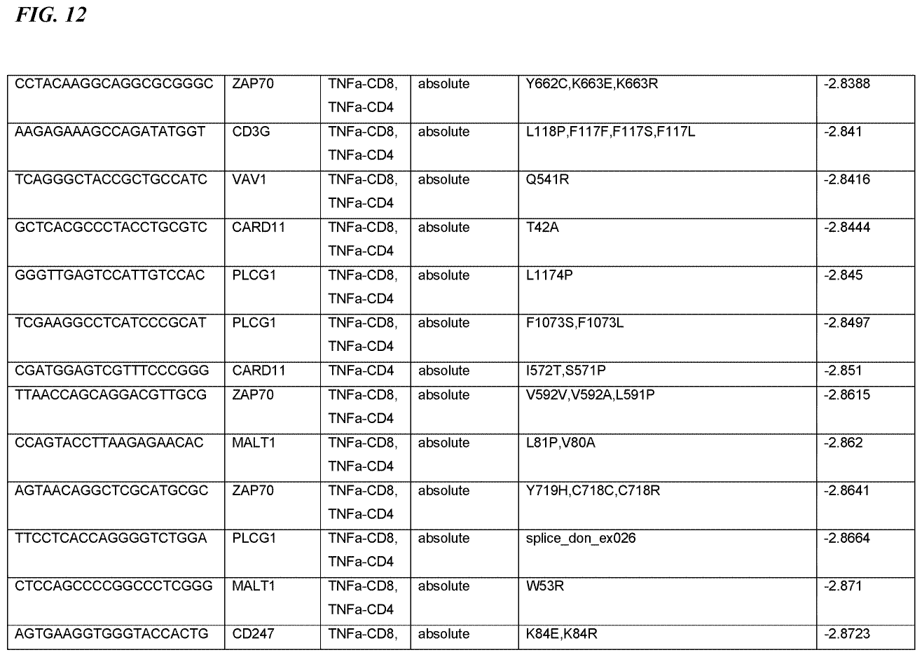

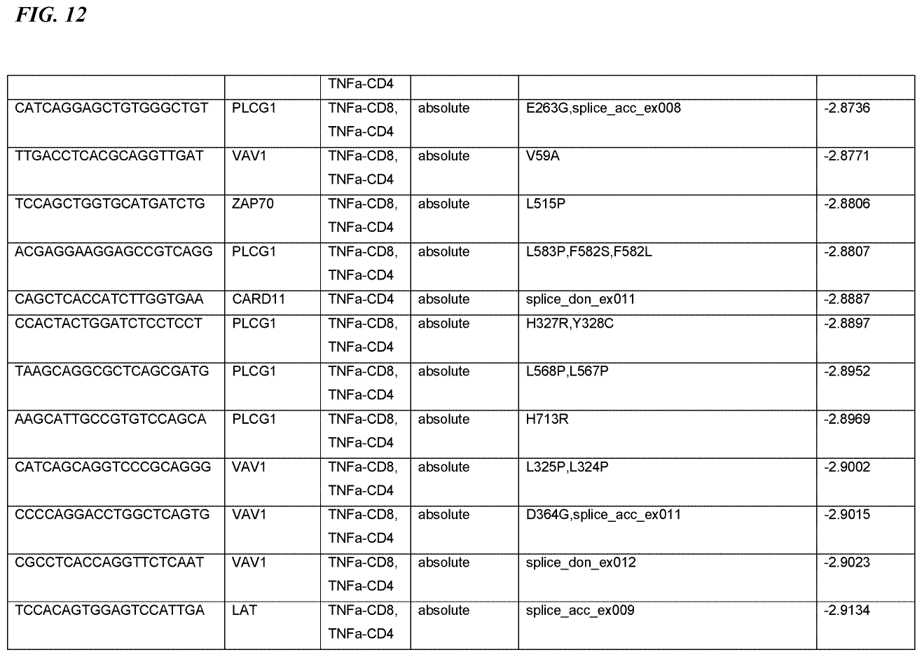

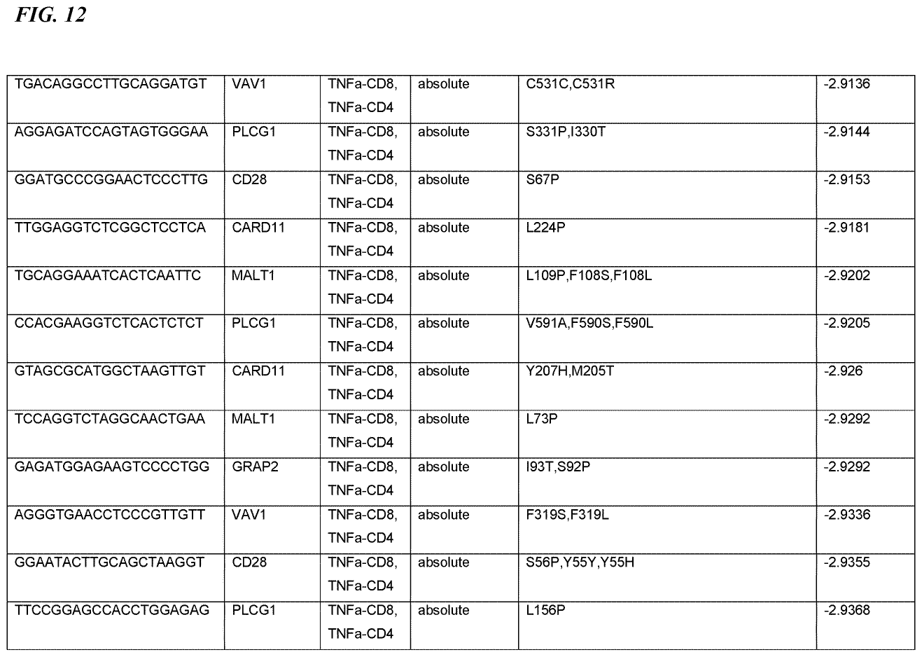

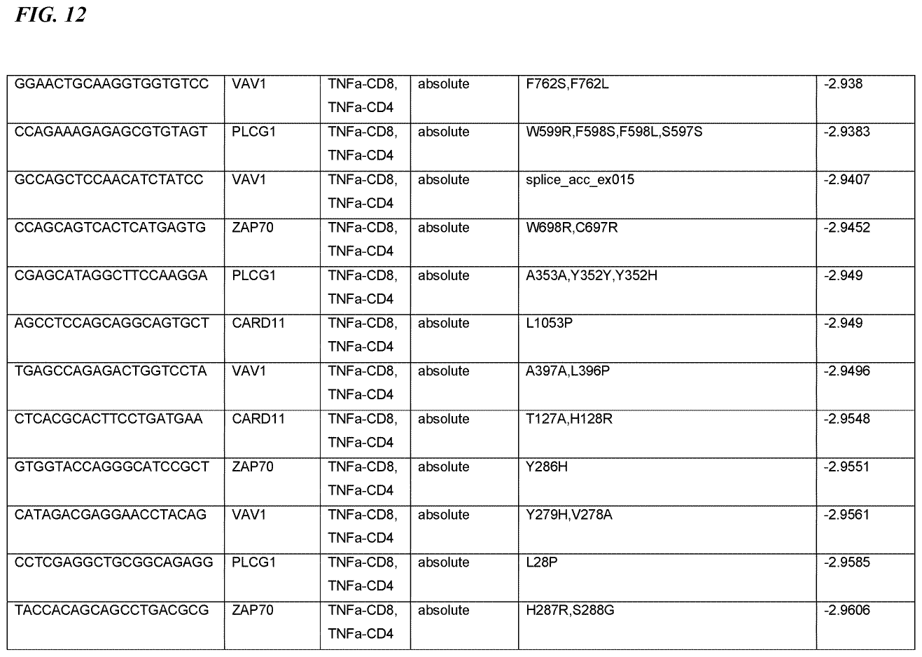

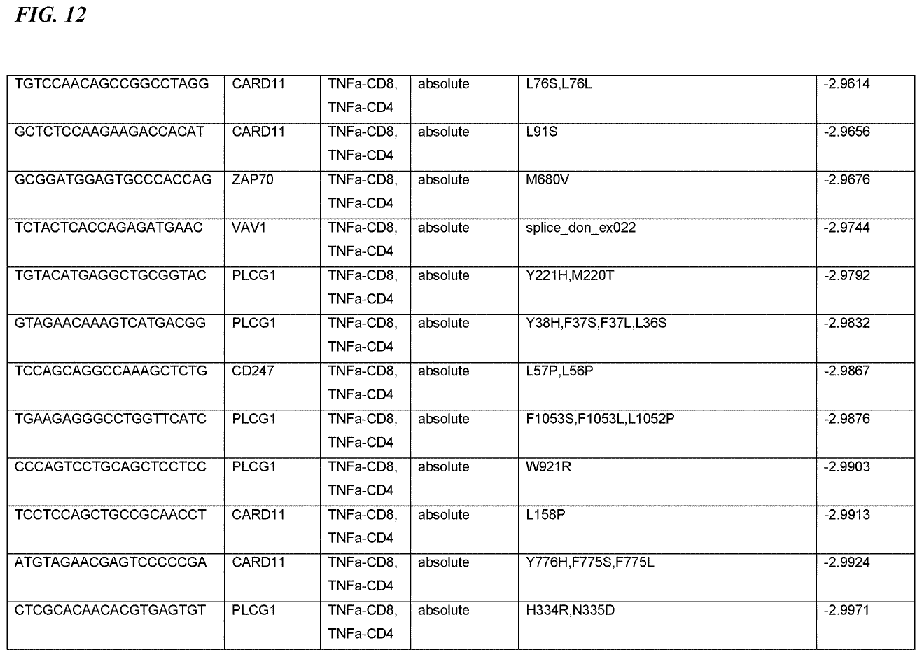

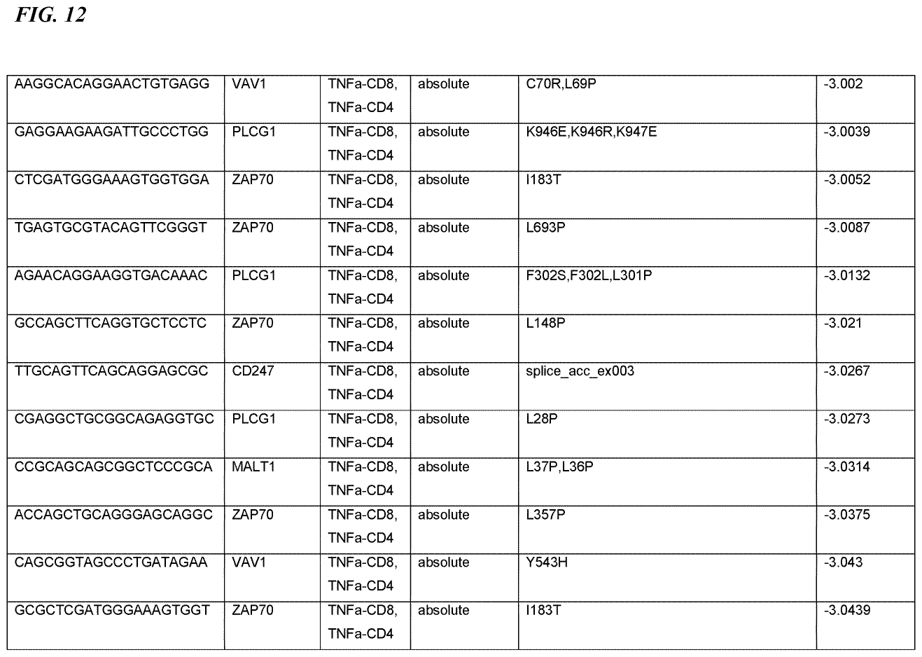

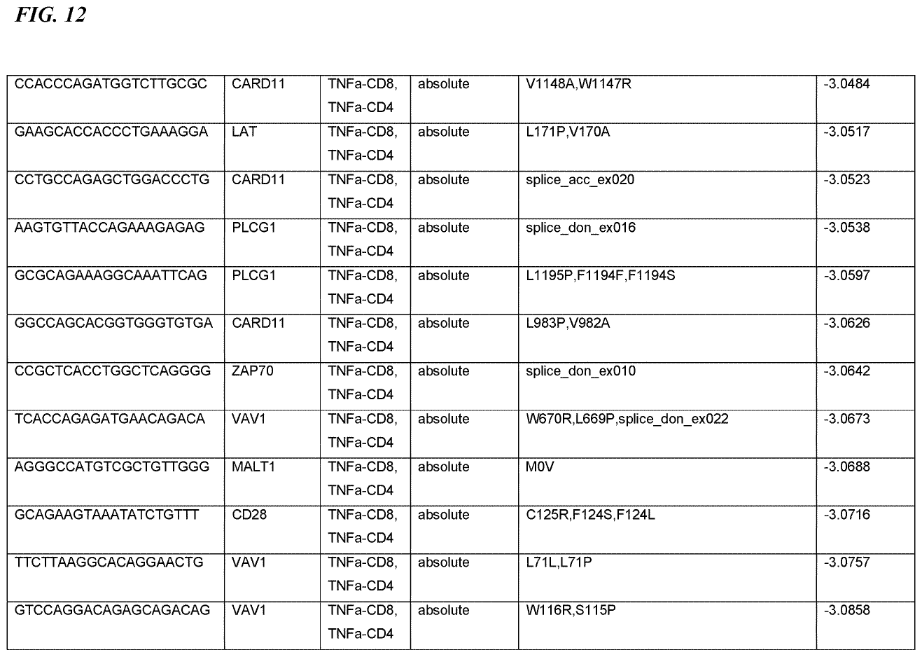

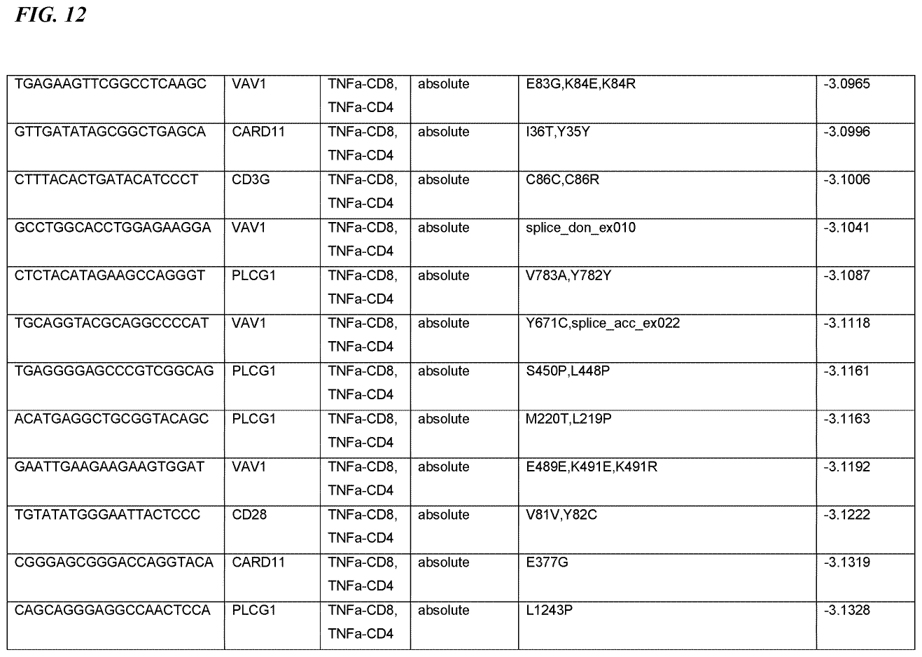

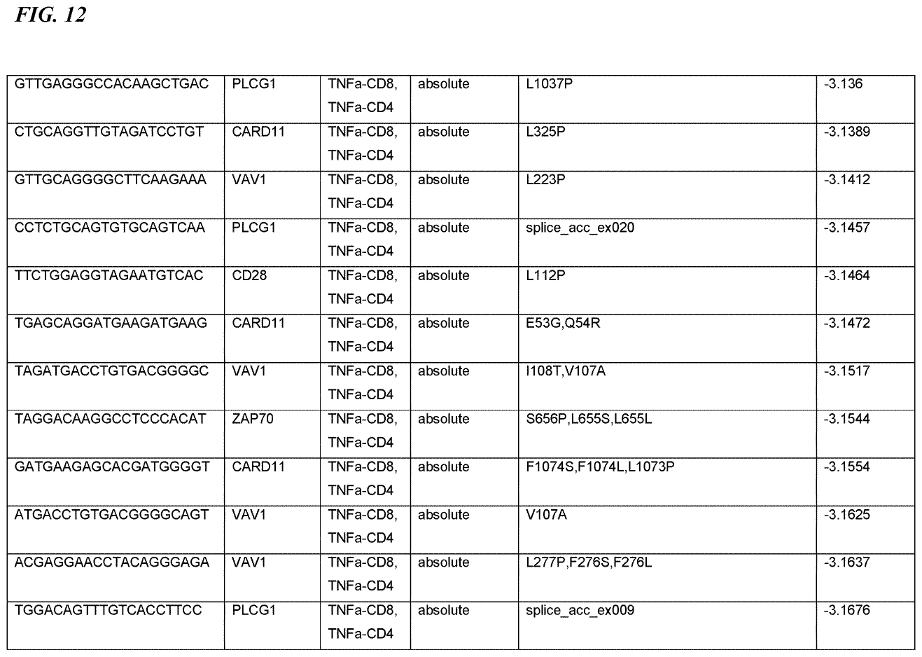

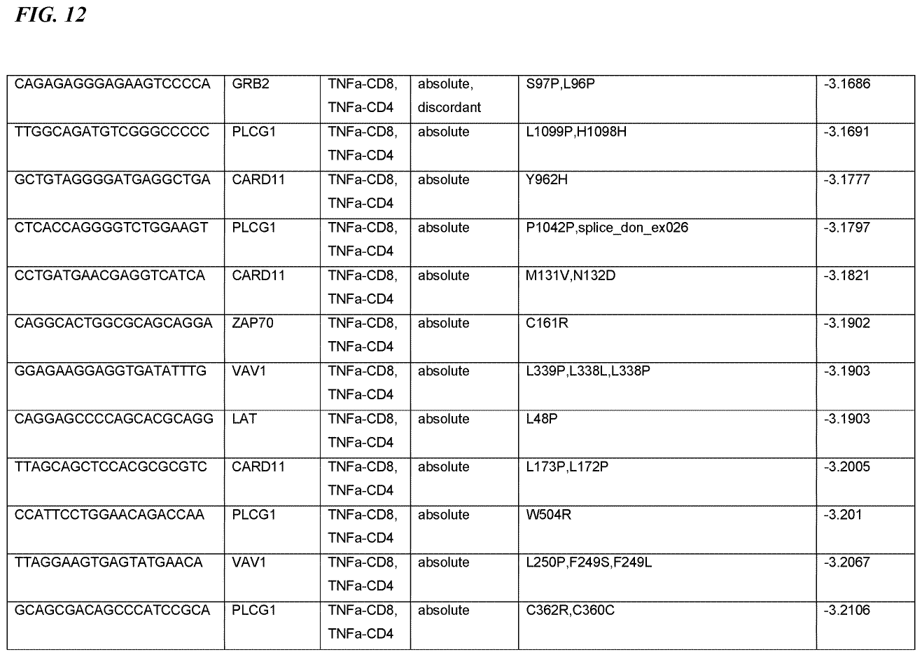

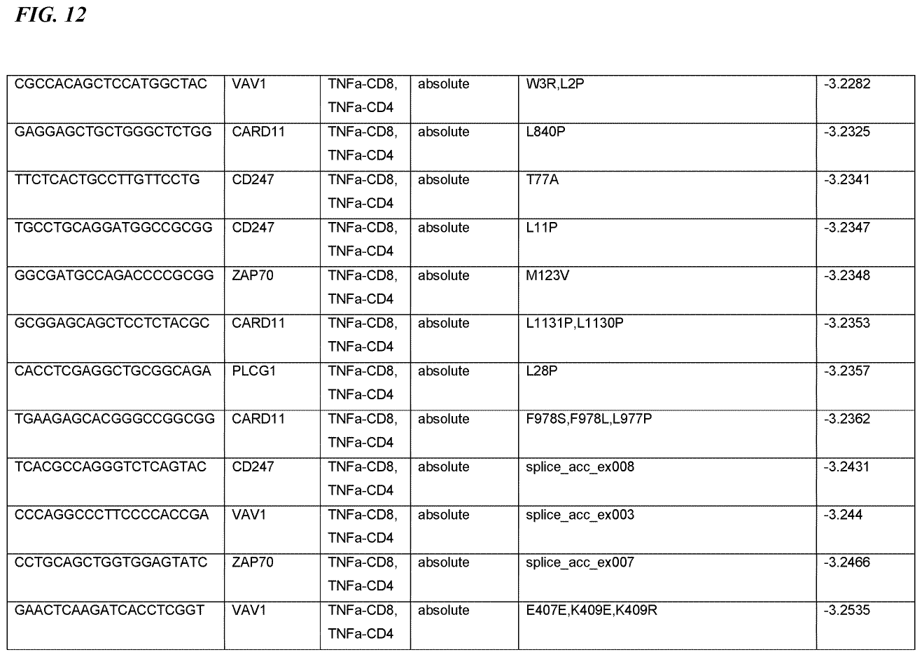

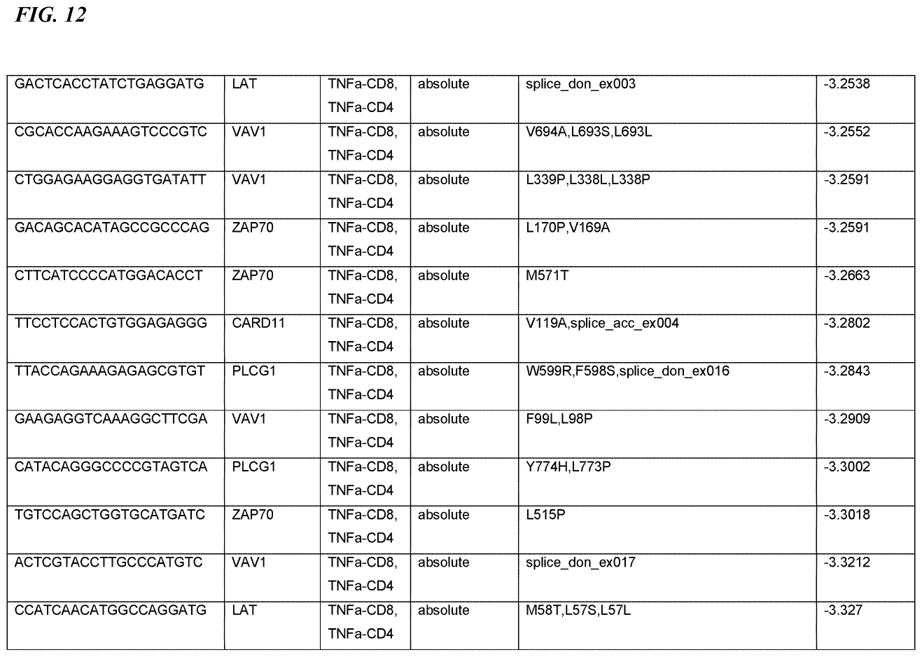

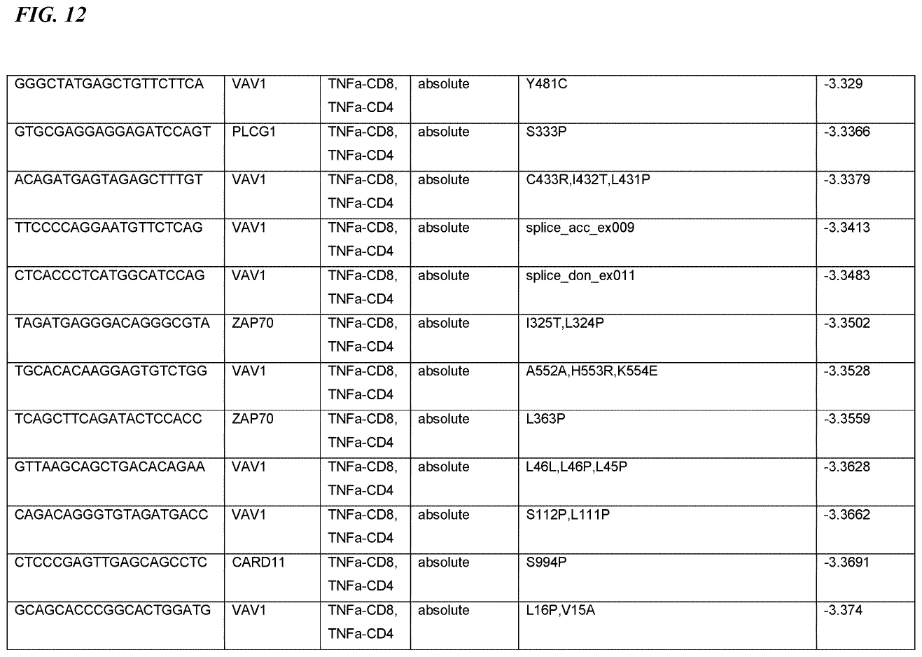

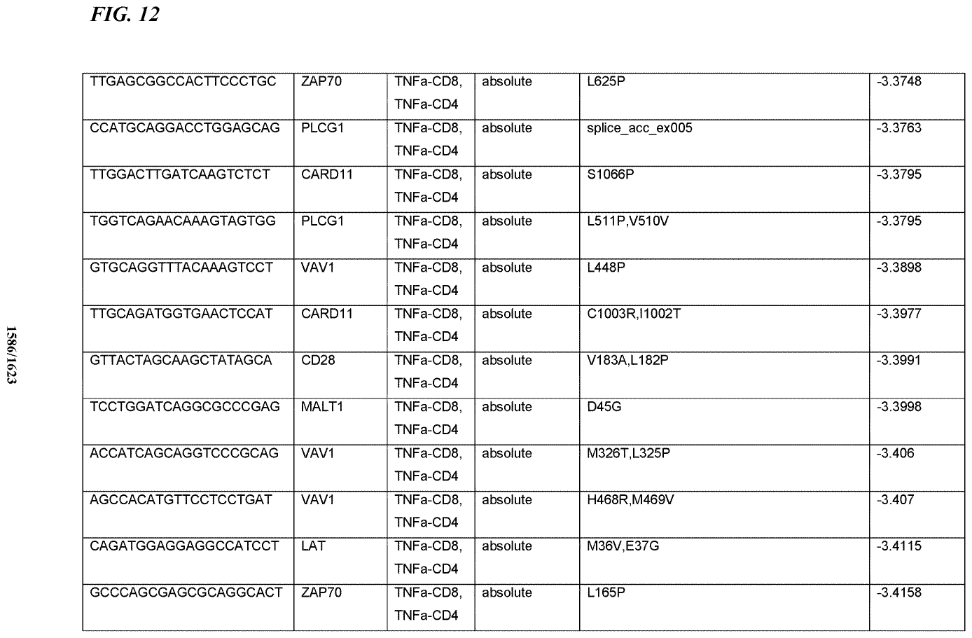

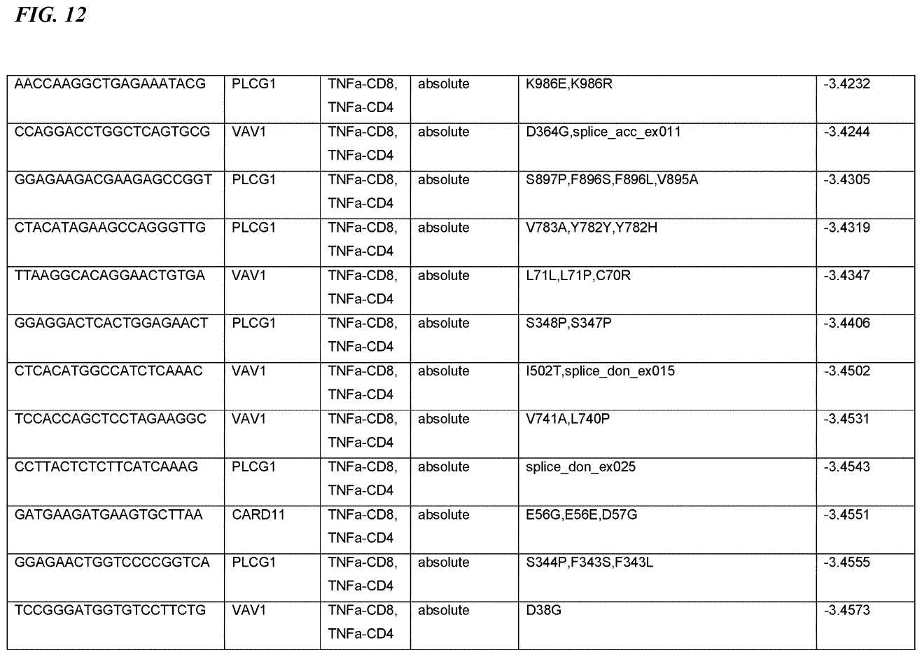

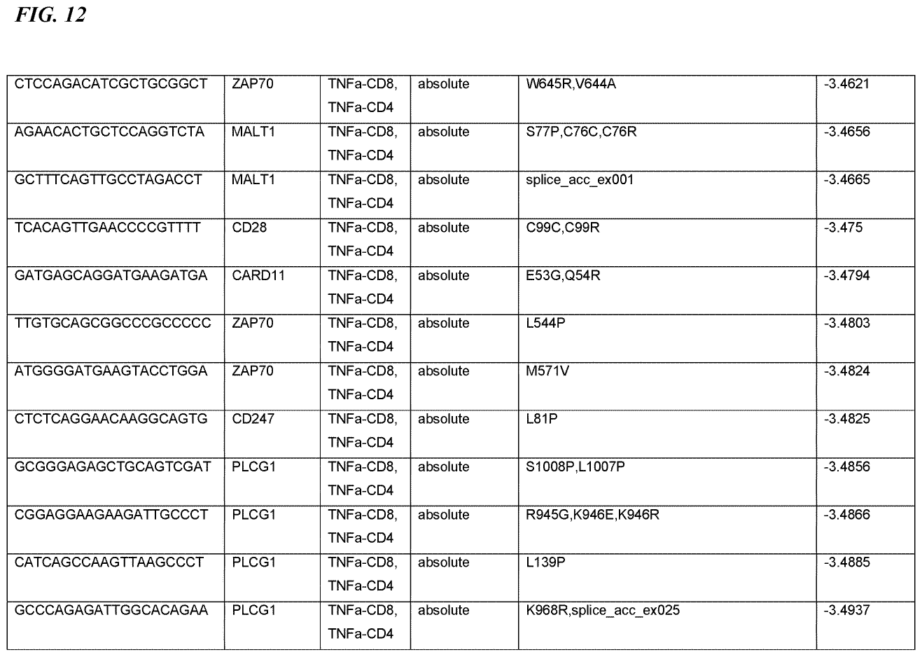

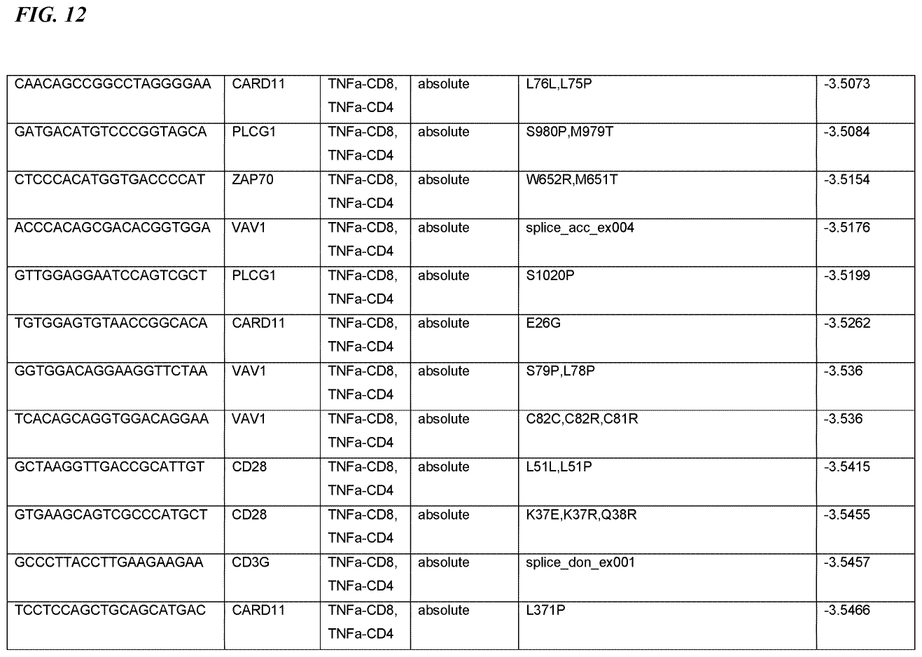

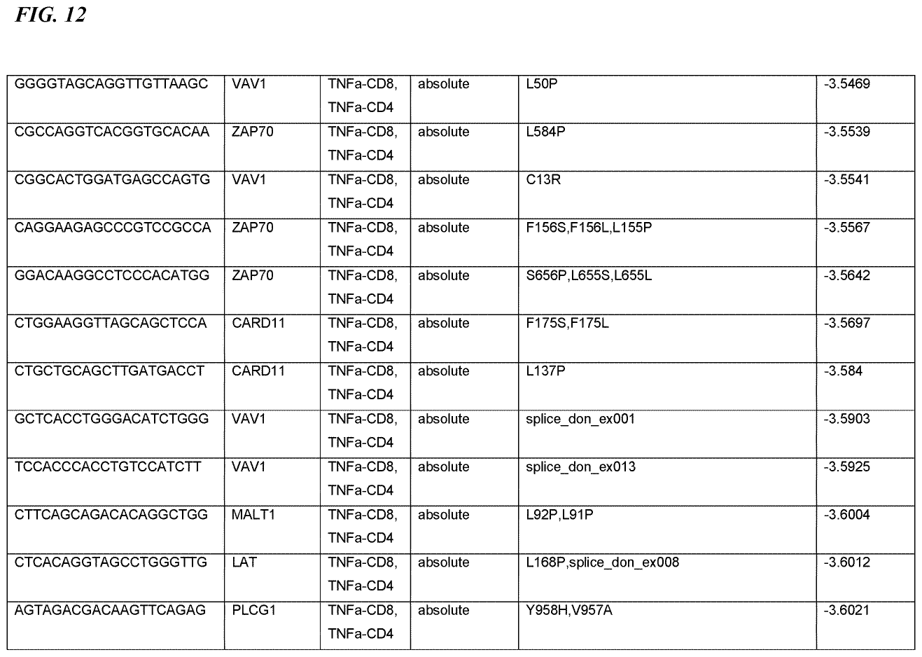

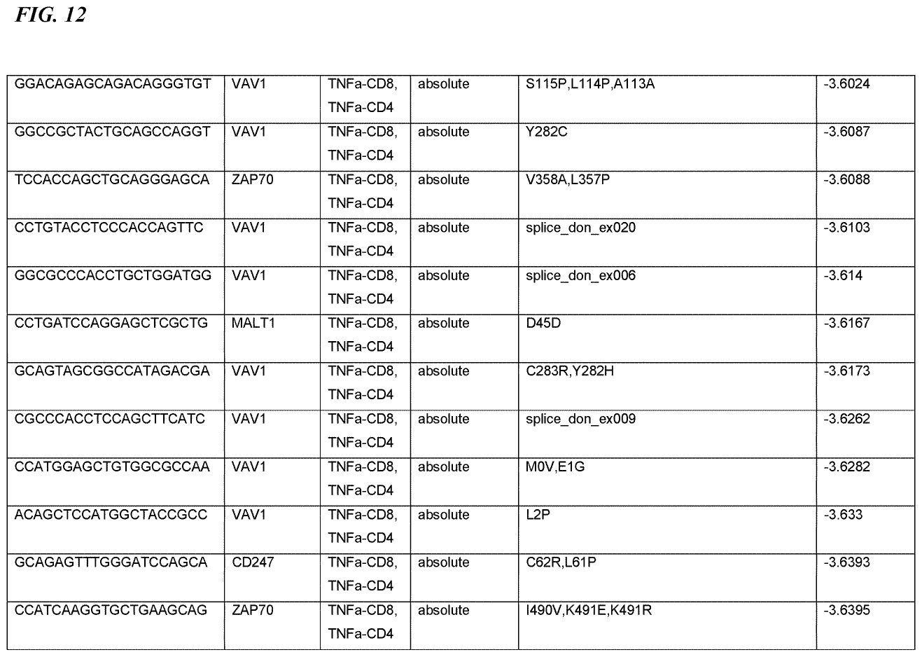

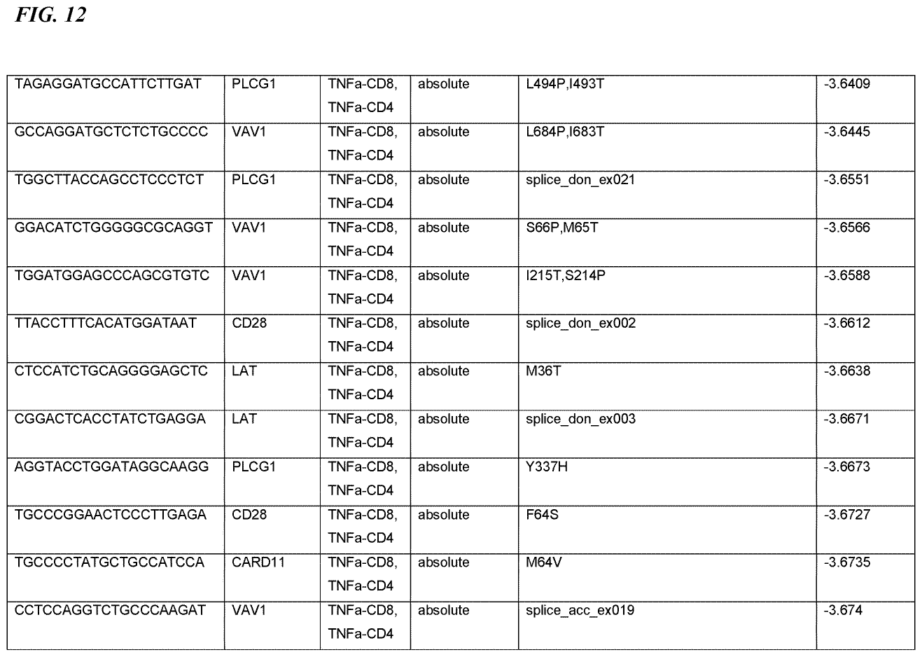

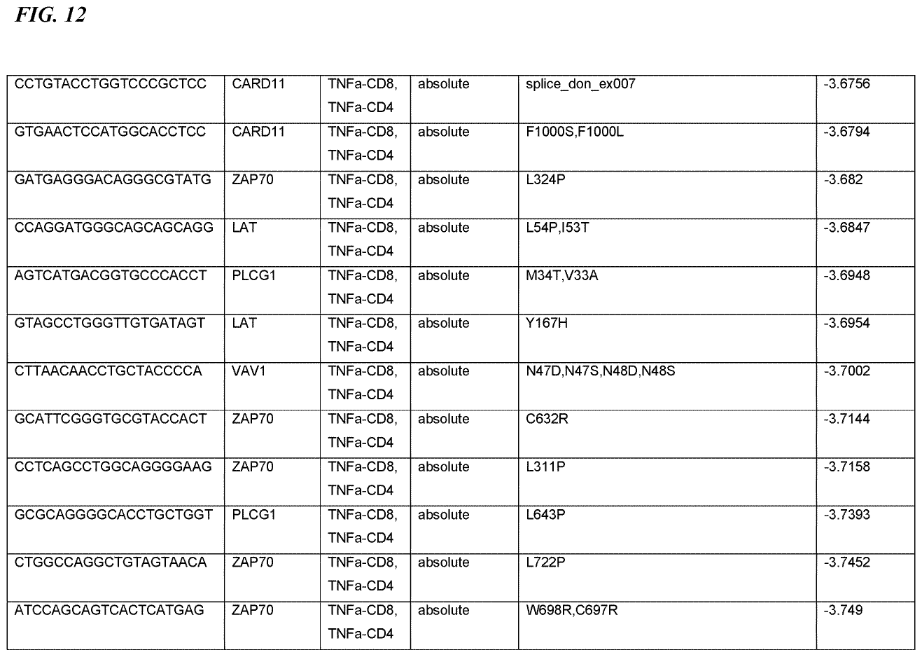

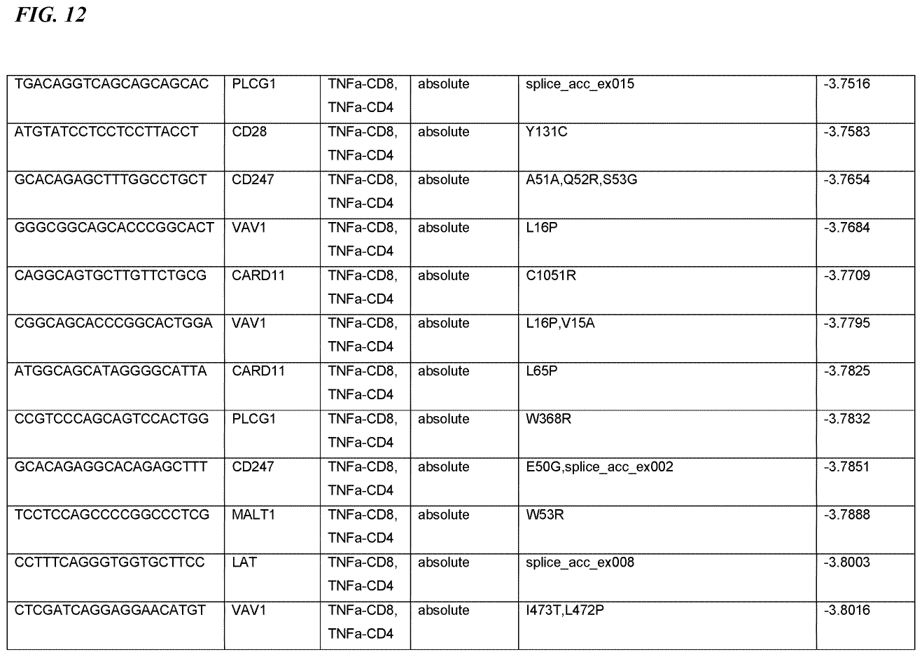

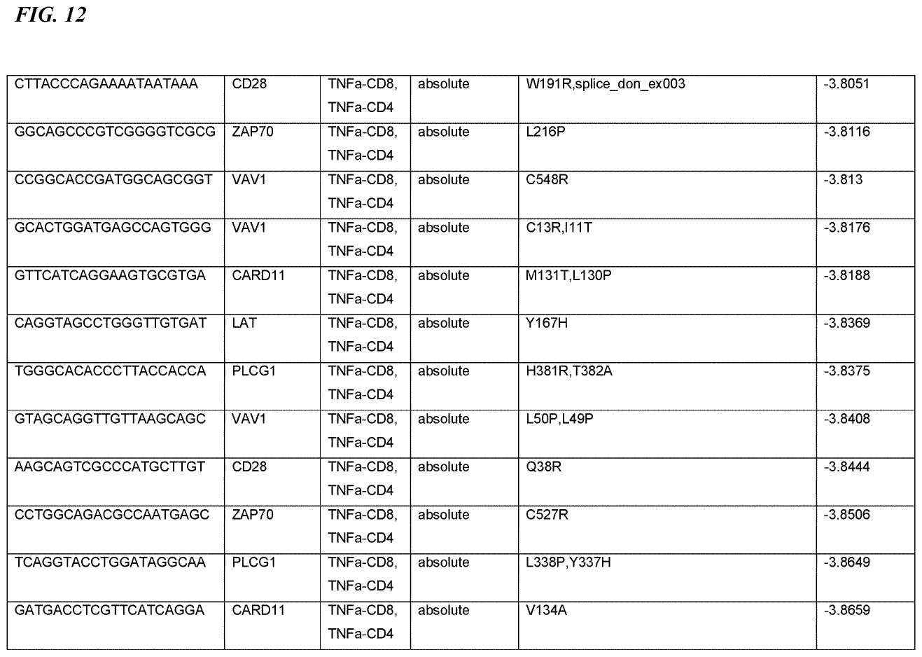

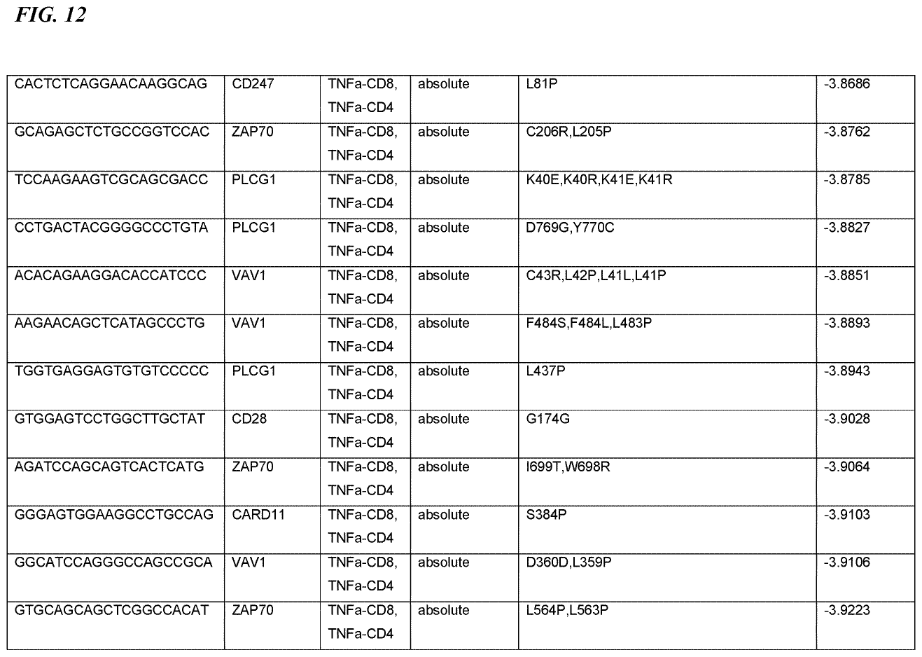

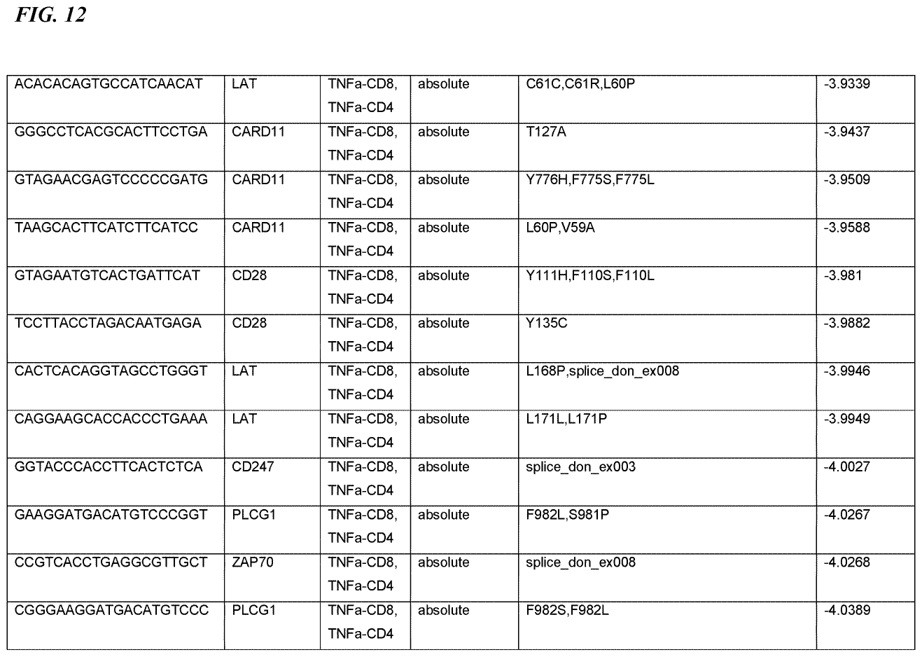

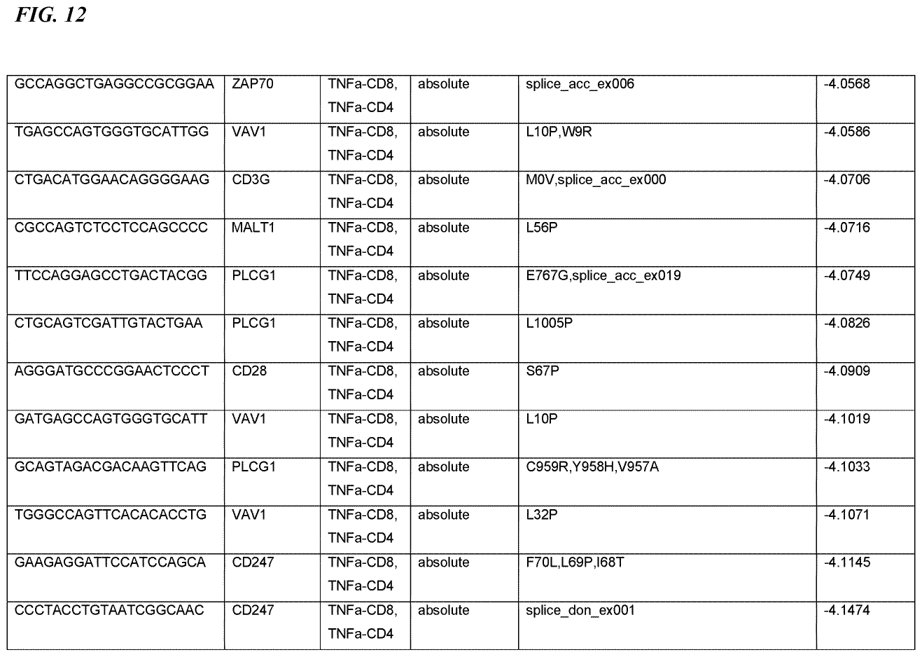

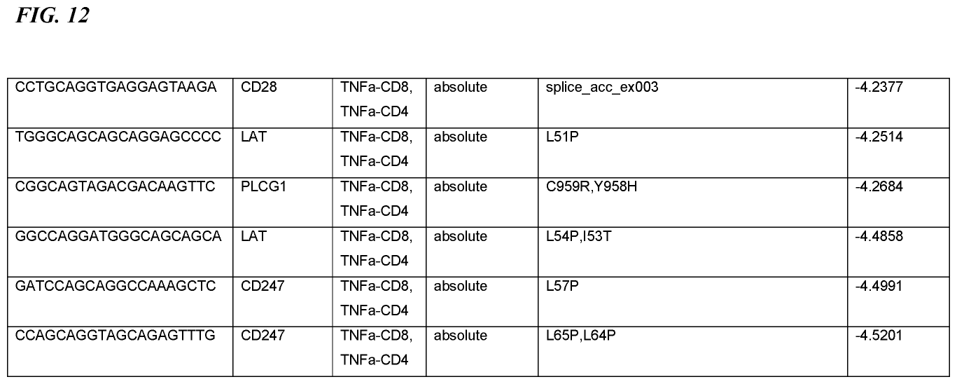

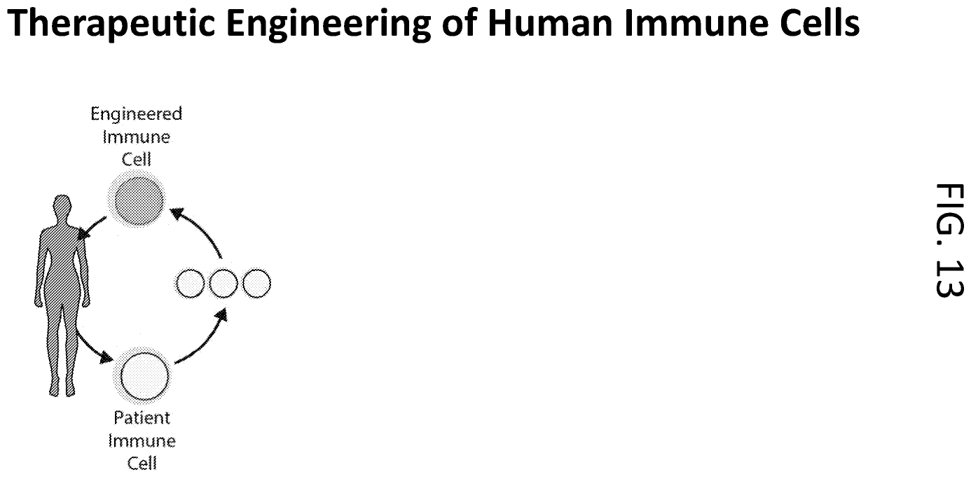

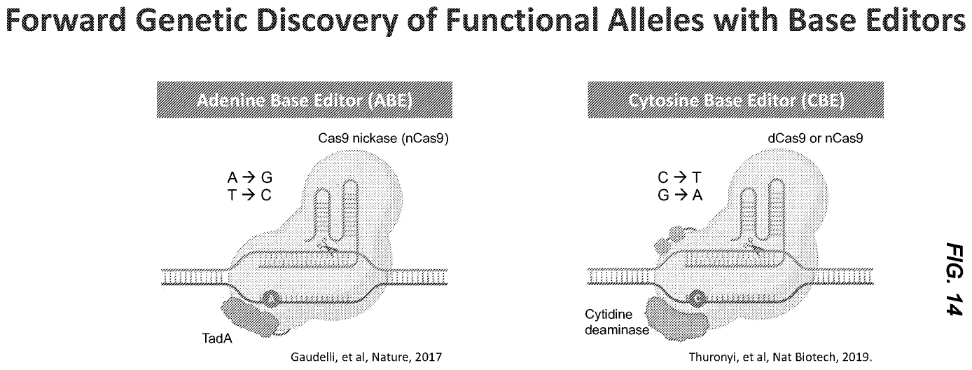

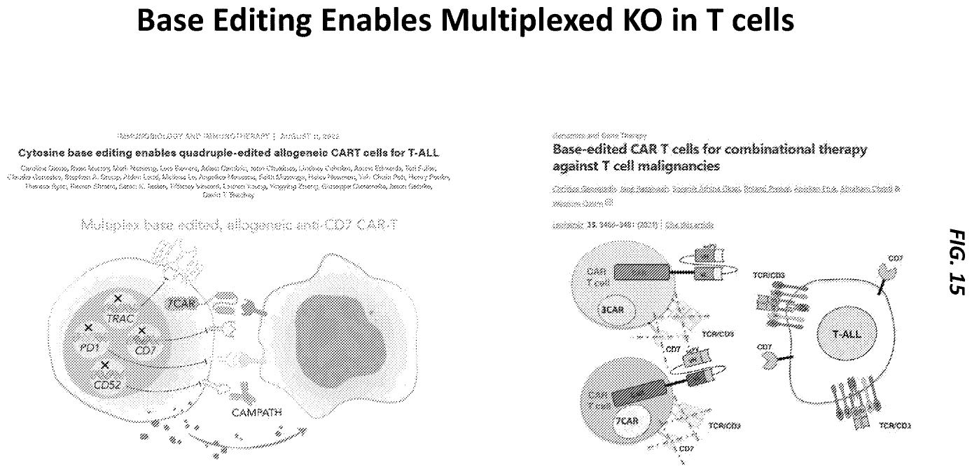

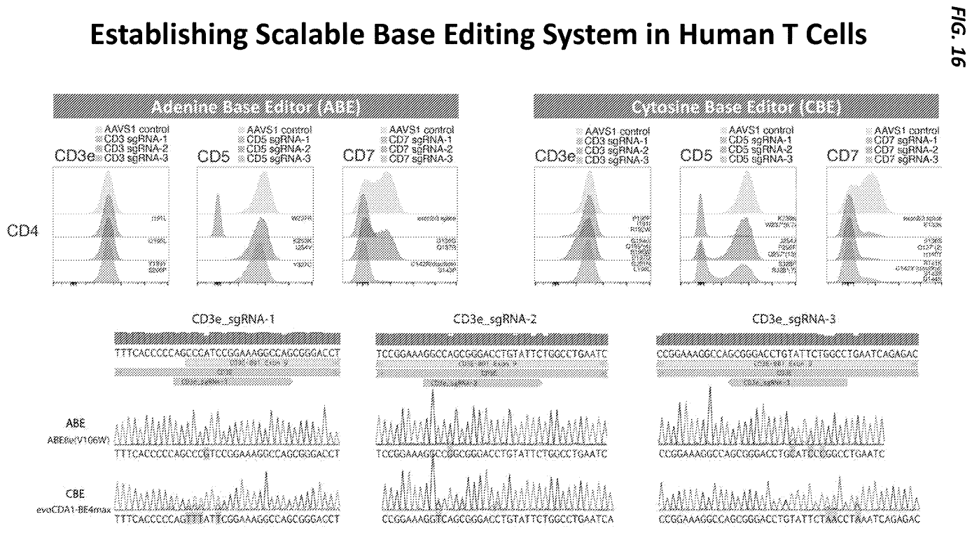

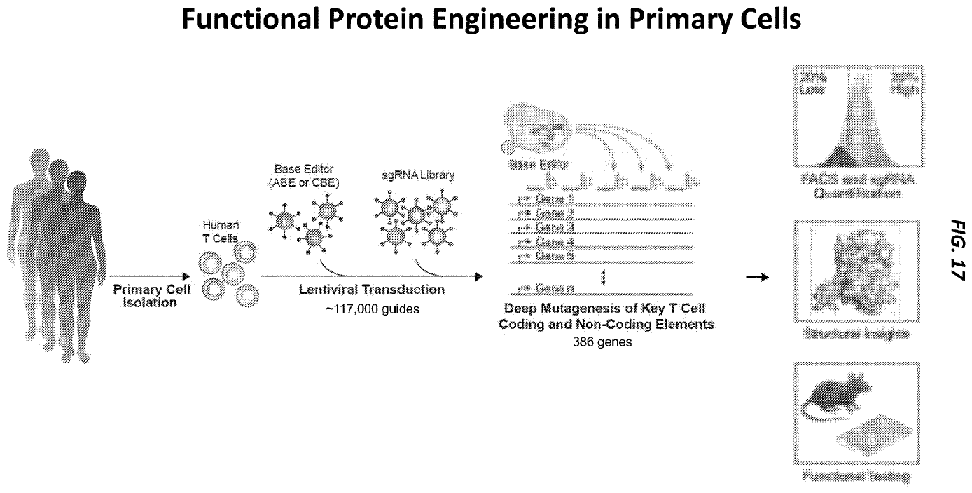

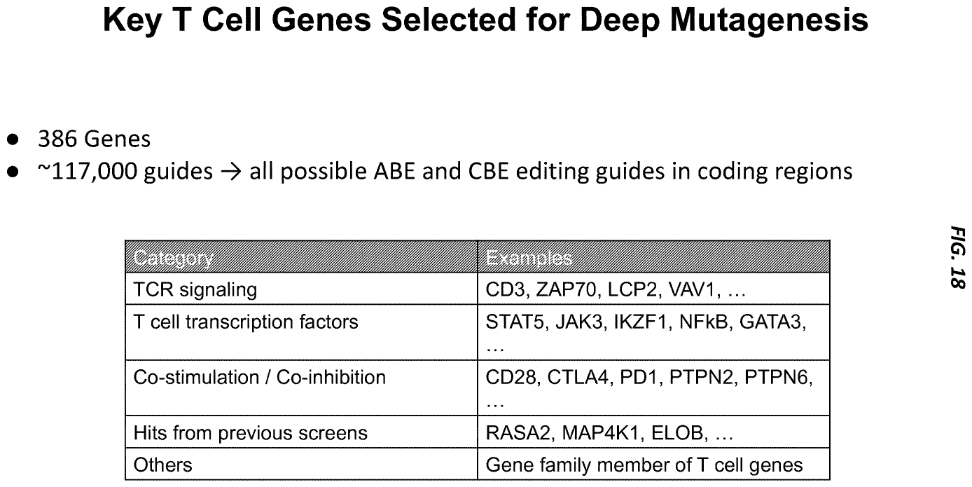

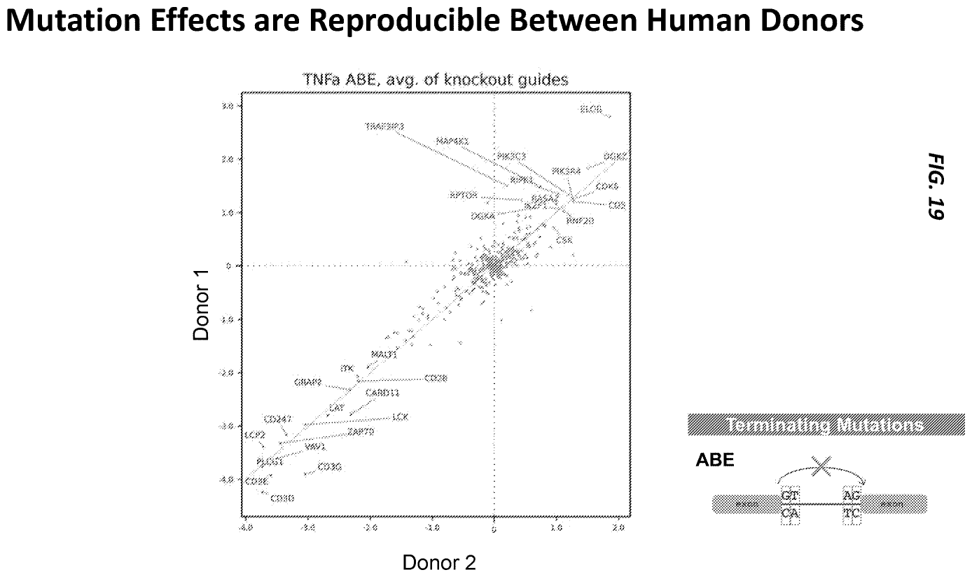

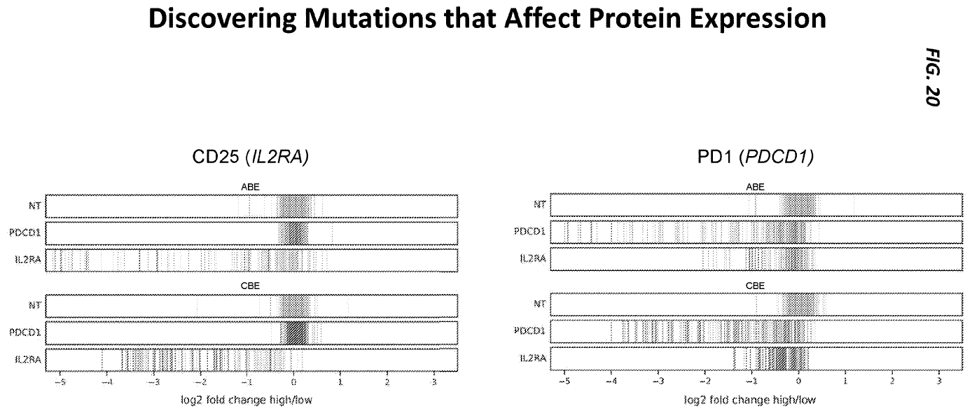

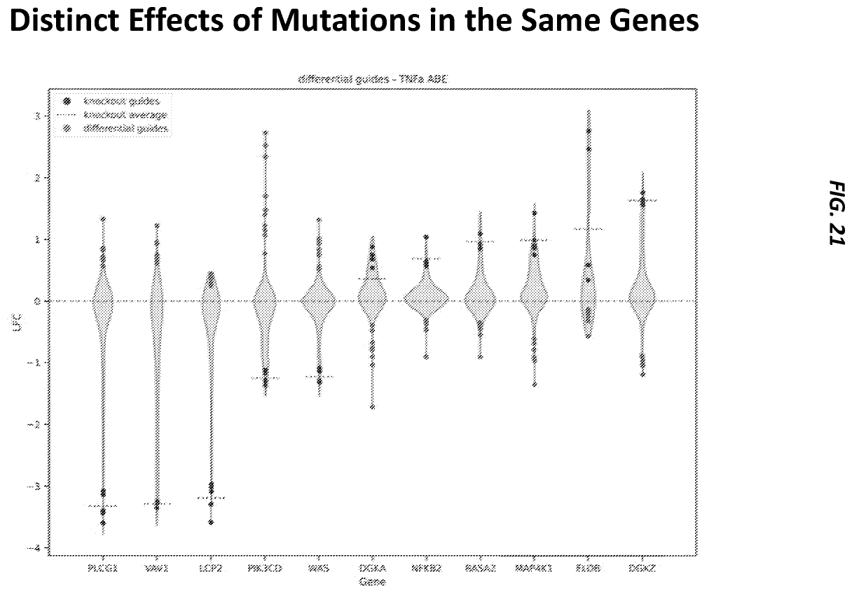

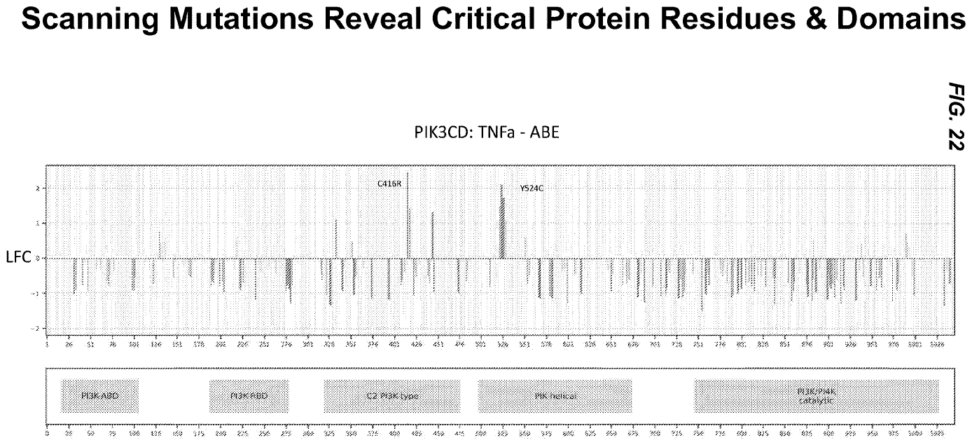

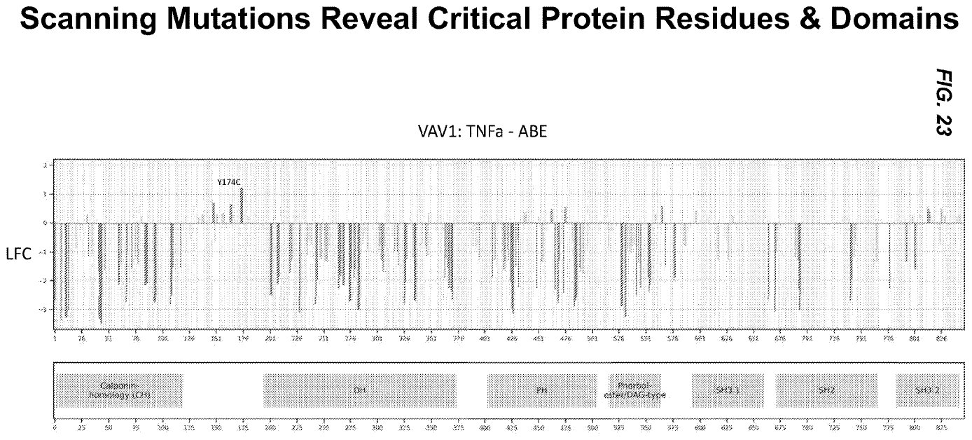

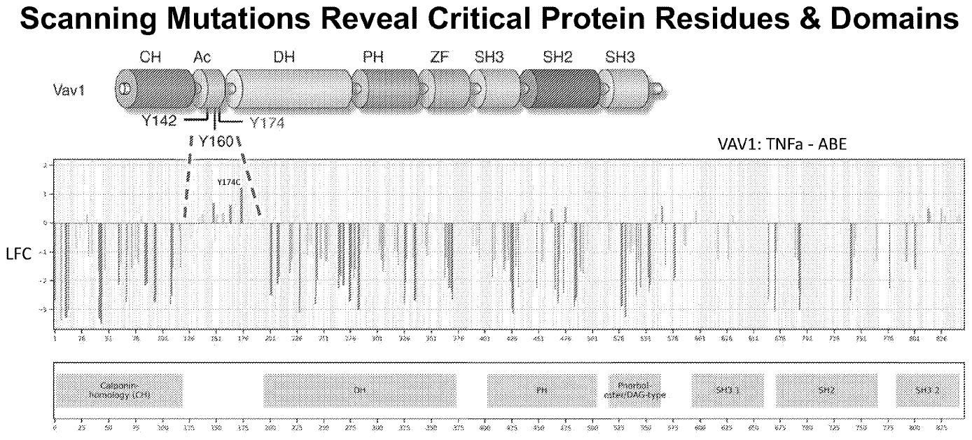

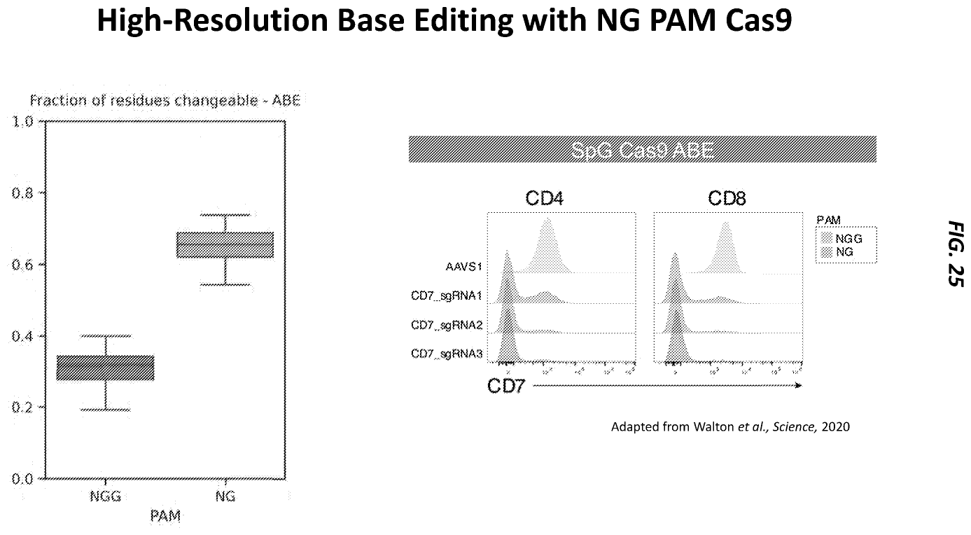

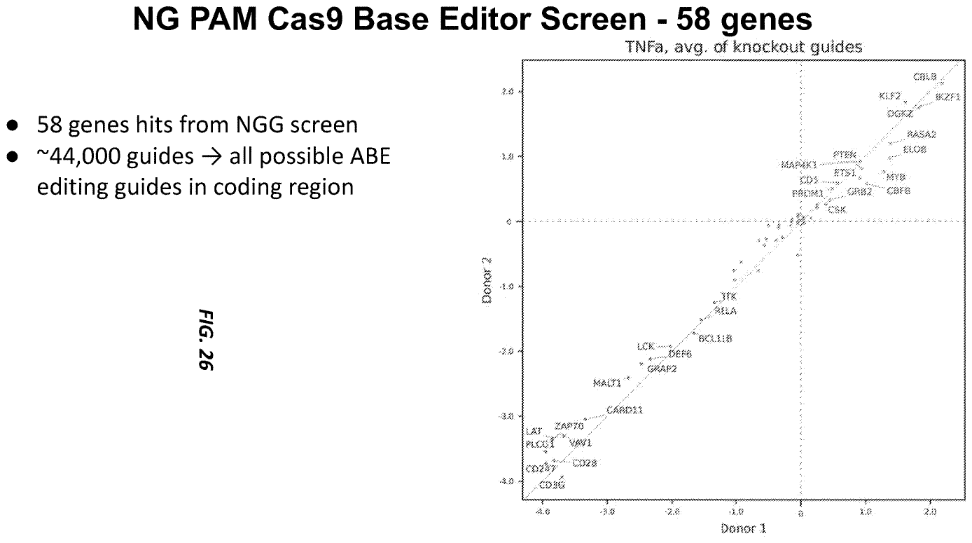

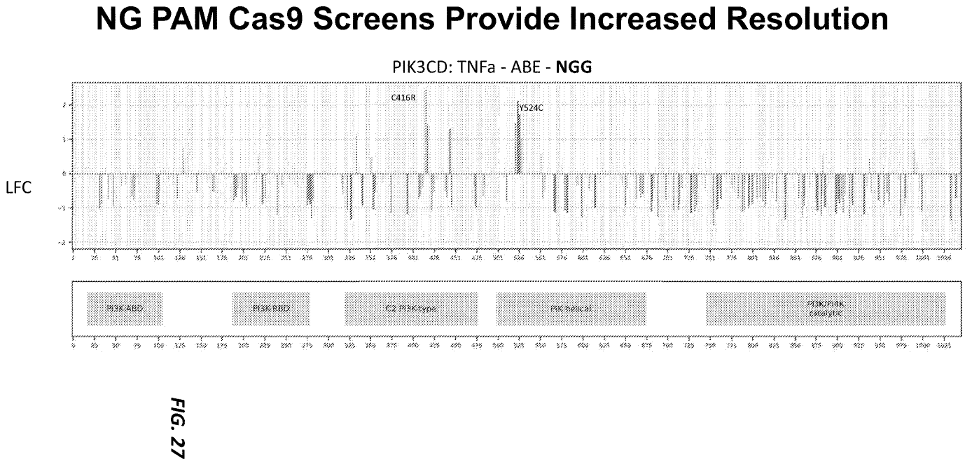

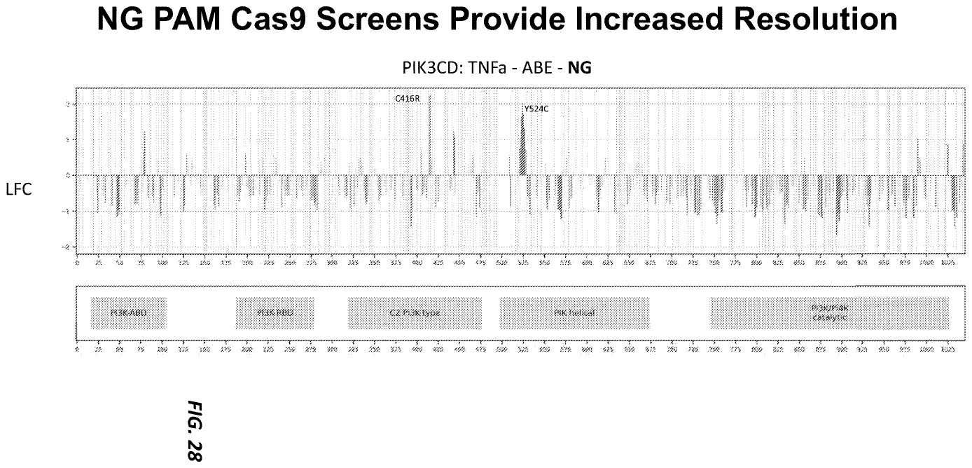

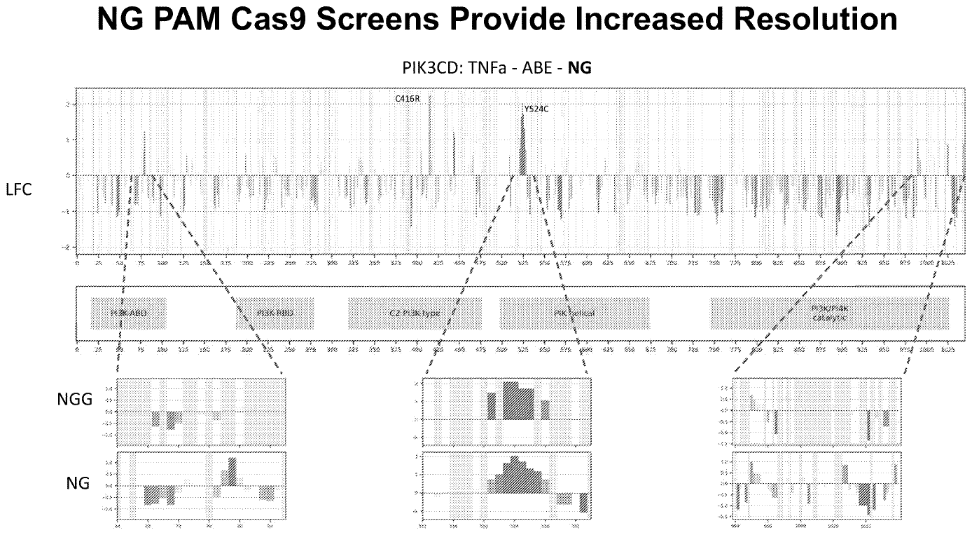

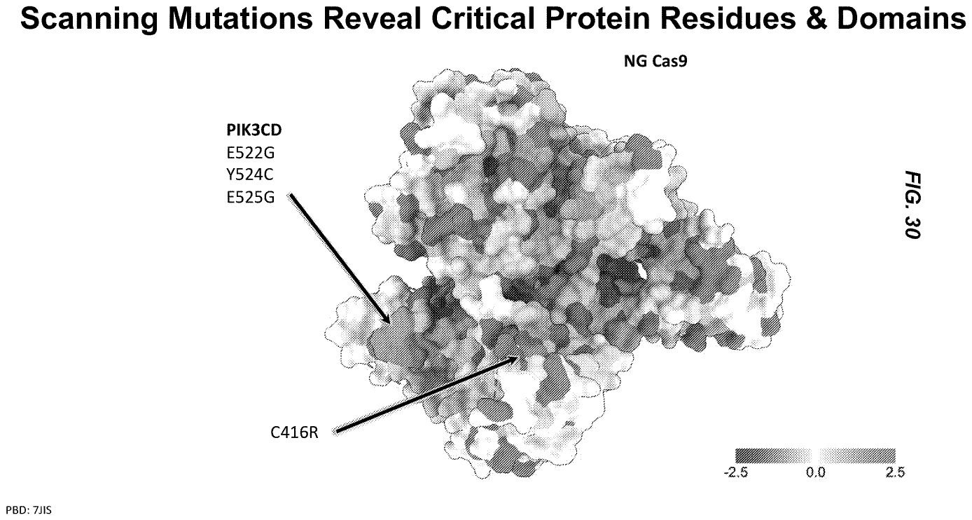

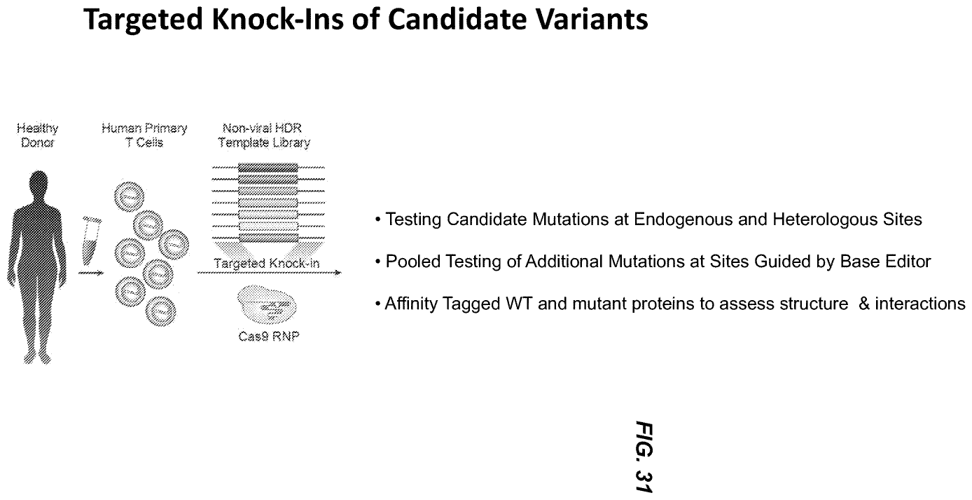

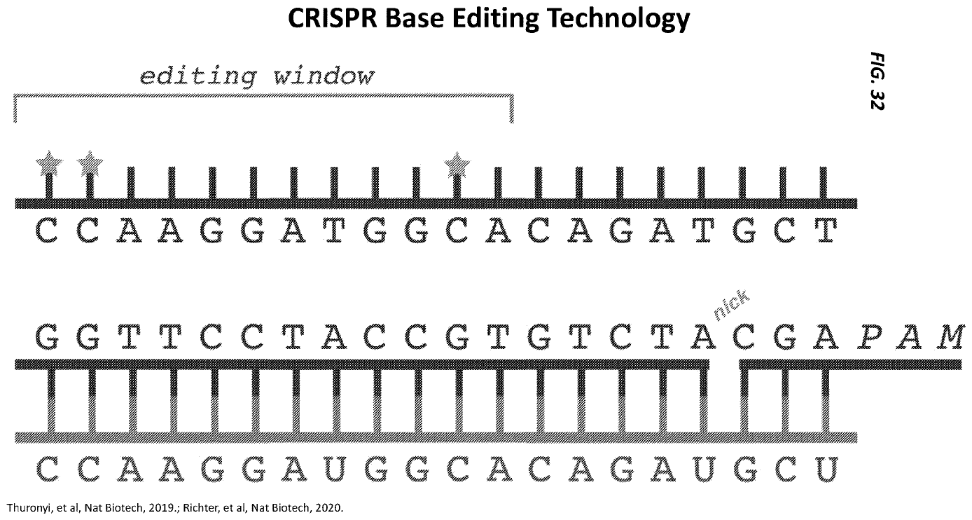

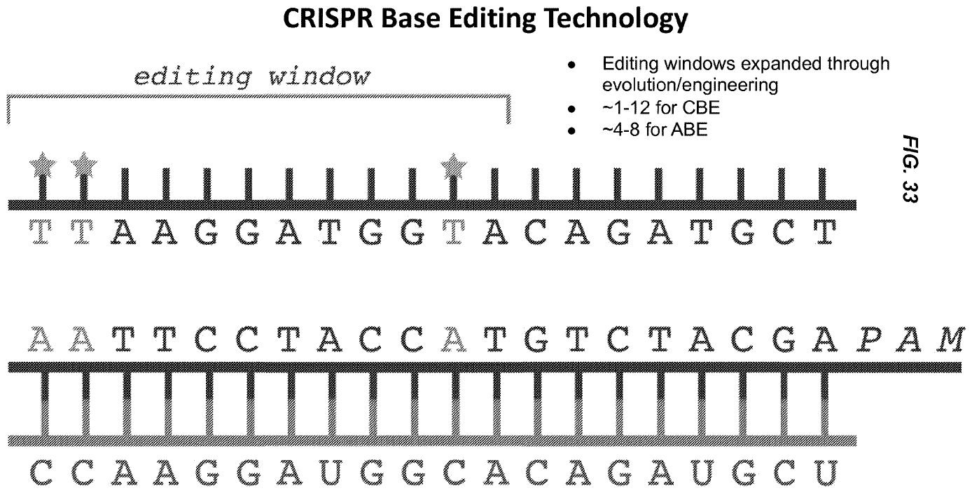

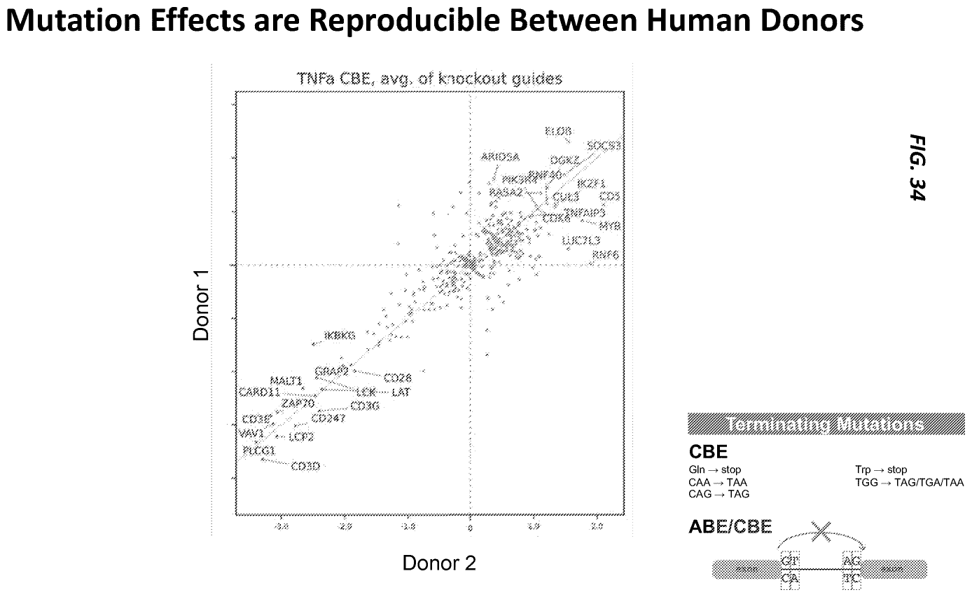

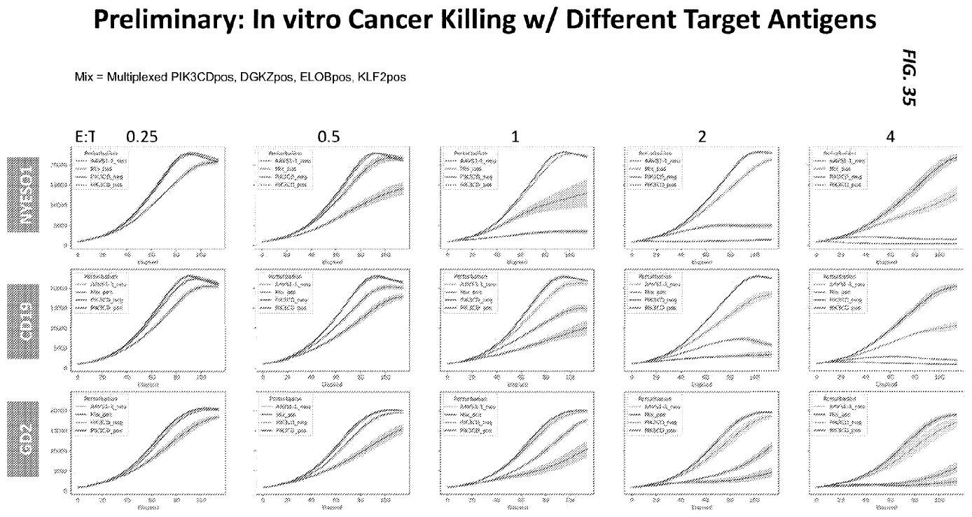

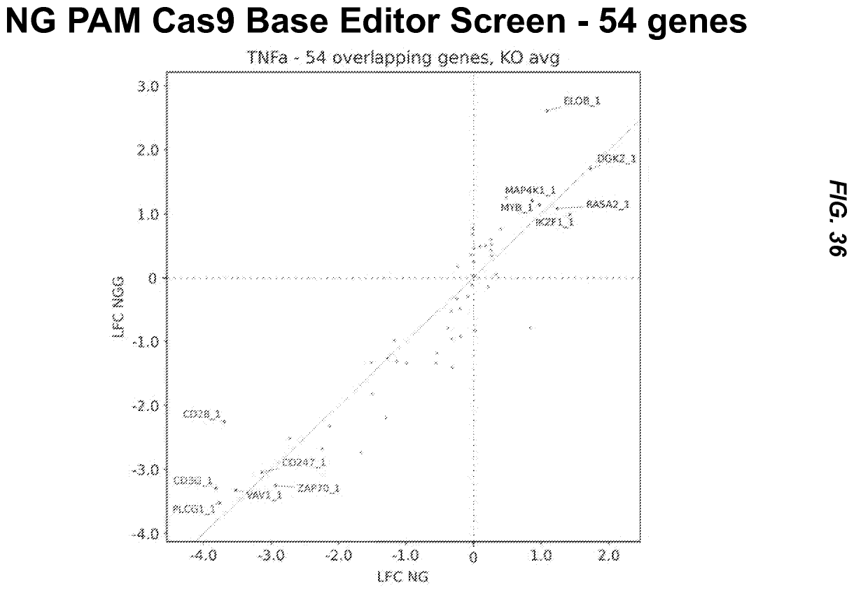

1. A method comprising:
   contacting T cells with one or more sgRNAs targeted to one or more coding regions in one or more genes and a base editor or nucleic acid encoding the base editor; and
   selecting one or more T cells that have an altered immune cell activity profile and optionally sorting the T cells based on the profile (or with single cell readouts (e.g., single cell RNA seq with capture of the sgRNA or base edit mutation) without selection).
2. The method of claim 1 wherein the cells are human cells.
3. The method of statement 1 or 2 wherein the human immune cells are contacted with a library of sgRNAs targeted to the one or more coding regions in a plurality of genes.
4. The method of any one of statements 1 to 3 wherein a library of viruses expresses the one or more sgRNAs.
5. The method of any one of statements 1 to 4 wherein a virus encodes the base editor.
6. The method of any one of statements 1 to 5 wherein the sgRNA and the nucleic acid encoding the base editor are on the same nucleic acid molecule.
7. The method of statement 4, 5 or 6 wherein the virus is a lentivirus, retrovirus, adenovirus, herpesvirus or adeno-associated virus.
8. The method of any one of statements 1 to 7 wherein the base editor comprises ABE.
9. The method of any one of statements 1 to 8 wherein the base editor comprises CBE.
10. The method of any one of statements 1 to 6 wherein the cells are selected for expression of TNFalpha, IL2 or IFNgamma, or any combination thereof.
11. The method of any one of statements 1 to 10 wherein the cells are sorted to separate cells that express TNFalpha, IL2 or IFNgamma, or any combination thereof, from cells that do not express TNFalpha, IL2 or IFNgamma, or any combination thereof.
12. The method of any one of statements 1 to 1 wherein the cells are selected for expression, including altered expression, of CD25 or PD1, or both (for example, sgRNA to base edit PD1 to lead to loss of protein expression).
13. The method of any one of statements 1 to 12 wherein the cells are sorted to separate cells that express CD25 or PD1, or both, from cells that do not express CD25 and/or PD1.
14. The method of any one of statements 1 to 13 wherein the sgRNAs comprise a plurality of the sequences in FIG. 1, 2, 3, 4 or 8 or a sequence with at least 85% nucleic acid sequence identity thereto.
15. The method of any one of statements 1 to 14 wherein the one or more coding regions that are altered are in a plurality of the genes in FIG. 1, 2, 3, 4 or 7 have or are modified at a site having one of the sequences in FIG. 1, 2, 3, 4 or 7 or a sequence with at least 85% nucleic acid sequence identity thereto.
16. A population of cells obtained by the method of any one of statements 1 to 15.
17. An isolated human cell selected by the method of any one of statements 1 to 15.
18. An isolated cell having a base edited gene in FIG. 7 which alters one or more activities of the cell.
19. The isolated cell of statement 18 which has increased T cell activity relative to a corresponding cell that lacks the base edited gene.
20. The isolated cell of statement 18 which has decreased T cell activity relative to a corresponding cell that lacks the base edited gene.
21. A method to introduce the altered genes described herein into cell therapies including viral transduction and variant knock-in (either to endogenous site or heterologous site).

1. A method comprising:
contacting human immune cells with one or more sgRNAs targeted to one or more coding regions in one or more genes and a base editor or nucleic acid encoding the base editor; and
selecting one or more human immune cells that have an altered immune cell activity and optionally sorting the immune cells based on the profile.

2. The method of claim 1, wherein the cells are T cells.

3. The method of claim 1 wherein the human immune cells are contacted with a library of sgRNAs targeted to the one or more coding regions in a plurality of genes.

4. The method of claim 1, wherein a library of viruses expresses the one or more sgRNAs.

5. The method of claim 1, wherein a virus encodes the base editor.

6. The method of claim 1, wherein the sgRNA and the nucleic acid encoding the base editor are on a different nucleic acid molecule.

7. The method of claim 4, wherein the virus is a lentivirus, retrovirus, adenovirus, herpesvirus or adeno-associated virus.

8. The method of claim 1, wherein the base editor comprises ABE or CBE.

9. (canceled)

10. The method of claim 1, wherein the cells are selected for altered expression of TNFalpha, IL2 or IFNgamma, or any combination thereof.

11. The method of claim 1, wherein the cells are sorted to separate cells that have altered expression of TNFalpha, IL2 or IFNgamma, or any combination thereof.

12. The method of claim 1, wherein the cells are selected for altered expression of CD25 or PD1, or both.

13. The method of claim 1, wherein the cells are sorted to separate cells that have altered expression of CD25 or PD1, or both, from cells that do not express CD25 and/or PDL.

14. The method of claim 1, wherein the sgRNAs comprise a plurality of the sequences in FIG. 1B, 2B, 3B, 4B or 8 or a sequence with at least 85% nucleic acid sequence identity thereto.

15. The method of claim 1, wherein the one or more coding regions that are altered are in one or more of the genes in FIG. 1A, 2A, 3A, 4A or 7 or have or are modified at a site in FIG. 1A, 2A, 3A, 4A, or 7 or a sequence with at least 85% nucleic acid sequence identity thereto.

16. (canceled)

17. An isolated human cell selected by the method of claim 1.

18. An isolated cell having a base edited gene in FIG. 1, 2, 3, 4 or 7 which alters one or more activities of the cell.

19. The isolated cell of claim 18 which has increased T cell activity relative to a corresponding cell that lacks the base edited gene.

20. The isolated cell of claim 18 which has decreased T cell activity relative to a corresponding cell that lacks the base edited gene.

21. (canceled)

22. The isolated cell of claim 18 which is modified at a site in FIG. 1A, 2A, 3A, 4A or 7 or a sequence with at least 85% nucleic acid sequence identity thereto.

23. A method to prevent, inhibit or treat cancer is a mammal, comprising administering to the mammal a composition having a plurality of the isolated cells of claim 18.

24.-26. (canceled)

27. The method of claim 23, wherein the cancer is a sarcoma, lymphoma, melanoma, or carcinoma, or is lung cancer, breast cancer, prostate cancer, pancreatic cancer or ovarian cancer.

28. The method of claim 23, wherein the cell is a CAR-T cell.

29.-30. (canceled)

31. A method to prevent, inhibit or treat an autoimmune disease is a mammal, comprising administering to the mammal a composition having a plurality of the isolated cell of claim 18.

32.-33. (canceled)

34. The method of claim 31, wherein the autoimmune disease is multiple sclerosis.

35.-36. (canceled)
